(19) United States
(12) Patent Application Publication
(10) Pub. No.: US 2024/0130147 A1
(43) Pub. Date: Apr. 18, 2024

(54) LIGHT EMITTING ELEMENT AND AMINE COMPOUND FOR THE SAME

(71) Applicant: Samsung Display Co., LTD., Yongin-si (KR)

(72) Inventor: Taku IMAIZUMI, Yokohama (JP)

(21) Appl. No.: 18/339,794

(22) Filed: Jun. 22, 2023

(30) Foreign Application Priority Data
Aug. 23, 2022 (KR) 10-2022-0105202

(51) Int. Cl. H10K 50/11; C07D 407/12; C07D 407/14; C07D 409/12; C07D 409/14; C09K 11/06; H10K 50/15; H10K 50/805; H10K 85/60

(52) U.S. Cl. CPC H10K 50/11; C07D 407/12; C07D 407/14; C07D 409/12; C07D 409/14; C09K 11/06; H10K 50/15; H10K 50/805; H10K 85/6574; H10K 85/6576

(57) ABSTRACT

Provided are a light emitting element and an amine compound for the same, wherein the light emitting element includes a first electrode, a second electrode on the first electrode, and at least one functional layer between the first electrode and the second electrode, the functional layer includes the amine compound represented by Formula 1 below, and thus the luminous efficiency and element life of the light emitting element may be improved.

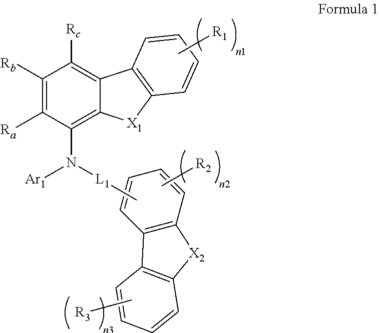

### CROSS-REFERENCE TO RELATED APPLICATION

This application claims priority to and the benefit of Korean Patent Application No. 10-2022-0105202, filed on Aug. 23, 2022, the entire contents of which are hereby incorporated by reference.

### BACKGROUND

### 1. Field

Embodiments of the present disclosure relate to a light emitting element and an amine compound for the same, and, for example, to a light emitting element including an amine compound in a functional layer.

### 2. Related Art

Recently, the development of an organic electroluminescence display device as an image display device is being actively conducted. The organic electroluminescence display device includes a so-called self-luminescent light emitting element in which holes and electrons injected from a first electrode and a second electrode recombine in an emission layer, and thus a luminescent material of the emission layer emits light to implement display.

In the application of a light emitting element to a display device, there is a demand for a light emitting element having high luminous efficiency and a long service life, and development on materials for a light emitting element capable of stably attaining such characteristics is being continuously required or desired.

For example, development on materials for a hole transport region having excellent hole transport properties and stability is being carried out in order to implement a light emitting element having high efficiency and a long service life.

### SUMMARY

Embodiments of the present disclosure provide a light emitting element exhibiting high efficiency and long service life characteristics and an amine compound included in the light emitting element.

An embodiment of the present disclosure provides a light emitting element including a first electrode, a second electrode on the first electrode, and at least one functional layer which is between the first electrode and the second electrode and includes an amine compound represented by Formula 1 below:

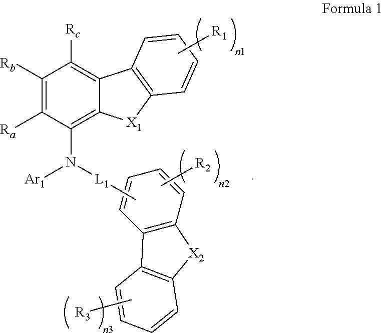

In Formula 1 above, X1 and X2 are each independently O or S, Ar₁ is a substituted or unsubstituted aryl group having 6 to 40 ring-forming carbon atoms, or a substituted or unsubstituted heteroaryl group having 2 to 40 ring-forming carbon atoms, Rₐ to R_{c} are each independently a hydrogen atom, a deuterium atom, a substituted or unsubstituted aryl group having 6 to 40 ring-forming carbon atoms, or a substituted or unsubstituted heteroaryl group having 2 to 40 ring-forming carbon atoms, at least one selected from Rₐ and R_{c} is a substituted or unsubstituted aryl group having 6 to 40 ring-forming carbon atoms, or a substituted or unsubstituted heteroaryl group having 2 to 40 ring-forming carbon atoms, R₁ to R₃ are each independently a hydrogen atom, a deuterium atom, a halogen atom, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms, L₁ is a direct linkage, a substituted or unsubstituted arylene group having 6 to 40 ring-forming carbon atoms, or a substituted or unsubstituted heteroarylene group having 2 to 40 ring-forming carbon atoms, the case where each of Rₐ to R_{c} includes a carbazole group is excluded, the case where each of Ar₁, Rₐ to R_{c}, L₁, and R₁ to R₃ includes a fluorenyl group, a benzonaphthofuranyl group, a benzonaphthothiophenyl group, and a benzobisbenzofuranyl group is excluded, n1 and n3 are each independently an integer of 0 to 4, and n2 is an integer of 0 to 3.

In an embodiment, the at least one functional layer may include an emission layer, a hole transport region between the first electrode and the emission layer, and an electron transport region between the emission layer and the second electrode, and the hole transport region may include the amine compound represented by Formula 1 above.

In an embodiment, the hole transport region may include a hole injection layer on the first electrode, and the hole transport layer on the hole injection layer, and the hole transport layer may include the amine compound represented by Formula 1 above.

In an embodiment, a layer, which is adjacent to the emission layer, among the plurality of layers included in the hole transport region may include the amine compound represented by Formula 1 above.

In an embodiment, the amine compound represented by Formula 1 above may be a monoamine compound.

In an embodiment, the amine compound represented by Formula 1 above may be represented by any one selected from among Formula 2-1 to Formula 2-3 below:

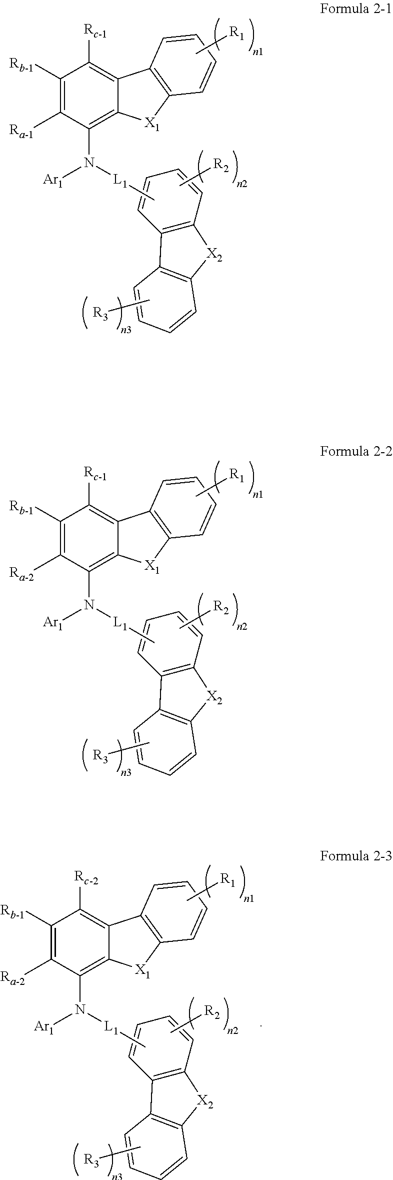

In Formula 2-1 to Formula 2-3 above, Rₐ₋₁, R_{b-1}, and R_{c-1} are each independently a hydrogen atom or a deuterium atom, Rₐ₋₂ and R_{c-2} are each independently a substituted or unsubstituted aryl group having 6 to 40 carbon atoms, or a substituted or unsubstituted heteroaryl group having 2 to 40 carbon atoms, and the case where each of Rₐ₋₂ and R_{c-2} includes a carbazole group, a fluorenyl group, a benzonaphthofuranyl group, a benzonaphthothiophenyl group, and a benzobisbenzofuranyl group is excluded.

In Formula 2-1 to Formula 2-3 above, the same as described with respect to Formula 1 above may be applied to X1, X2, Ar₁, R₁ to R₃, L₁, n1, n2 and n3.

In an embodiment, the amine compound represented by Formula 1 above may be represented by any one selected from among Formula 3-1 to Formula 3-4 below:

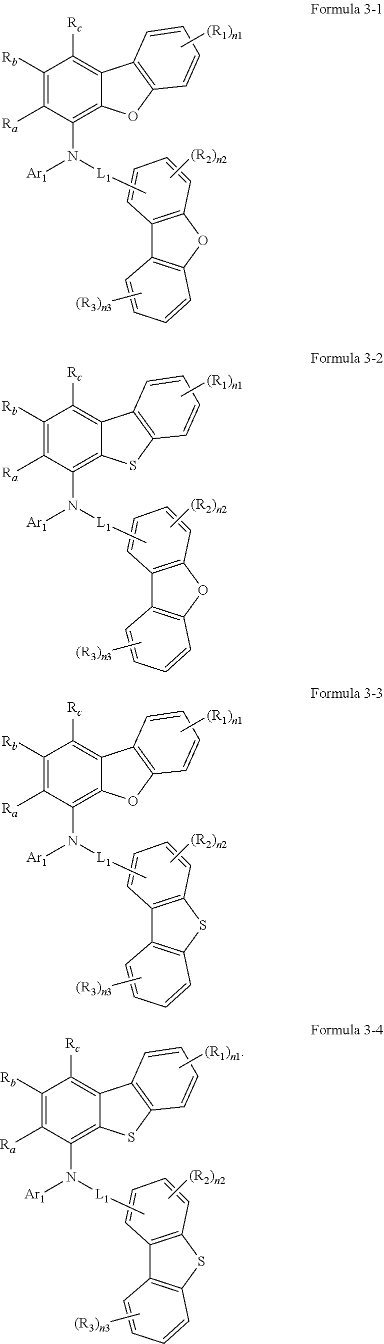

In Formula 3-1 to Formula 3-4 above, the same as described with respect to Formula 1 above may be applied to Ar₁, Rₐ to R_{c}, R₁ to R₃, L₁, n1, n2, and n3.

In an embodiment, the amine compound represented by Formula 1 above may be represented by any one selected from among Formula 4-1 to Formula 4-4 below:

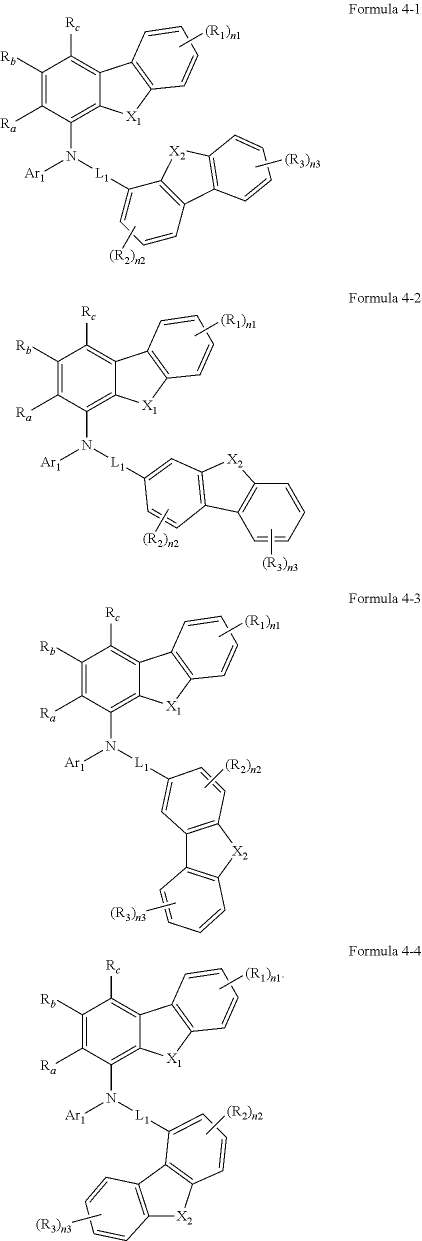

In Formula 4-1 to Formula 4-4 above, the same as described with respect to Formula 1 above may be applied to X₁, X₂, Ar₁, Rₐ to R_{c}, R₁ to R₃, L₁, n1, n2, and n3.

In an embodiment, in Formula 1 above, at least one selected from Rₐ and R_{c} may be a substituted or unsubstituted phenyl group, a substituted or unsubstituted naphthyl group, a substituted or unsubstituted biphenyl group, a substituted or unsubstituted phenanthrenyl group, a substituted or unsubstituted dibenzofuranyl group, or a substituted or unsubstituted dibenzothiophenyl group.

In an embodiment, in Formula 1 above, L₁ may be a direct linkage, or a substituted or unsubstituted p-phenylene group.

In an embodiment, in Formula 1 above, Ar₁ may be a substituted or unsubstituted phenyl group, a substituted or unsubstituted biphenyl group, a substituted or unsubstituted terphenyl group, a substituted or unsubstituted a naphthyl group, a substituted or unsubstituted phenanthrene group, a substituted or unsubstituted dibenzofuranyl group, or a substituted or unsubstituted dibenzothiophenyl group.

In an embodiment, in Formula 1 above, R₂ and R₃ may be each independently a hydrogen atom, a deuterium atom, or a substituted or unsubstituted phenyl group.

In an embodiment of the present disclosure, an amine compound is represented by Formula 1 above.

### BRIEF DESCRIPTION OF THE FIGURES

The accompanying drawings are included to provide a further understanding of the subject matter of the present disclosure, and are incorporated in and constitute a part of this specification. The drawings illustrate example embodiments of the present disclosure and, together with the description, serve to explain principles of the present disclosure. In the drawings:

FIG. 1 is a plan view illustrating a display device according to an embodiment of the present disclosure;

FIG. 2 is a cross-sectional view of a display device according to an embodiment of the present disclosure;

FIG. 3 is a cross-sectional view schematically illustrating a light emitting element according to an embodiment of the present disclosure;

FIG. 4 is a cross-sectional view schematically illustrating a light emitting element according to an embodiment of the present disclosure;

FIG. 5 is a cross-sectional view schematically illustrating a light emitting element according to an embodiment of the present disclosure;

FIG. 6 is a cross-sectional view schematically illustrating a light emitting element according to an embodiment of the present disclosure;

FIG. 7 is a cross-sectional view of a display device according to an embodiment of the present disclosure;

FIG. 8 is a cross-sectional view of a display device according to an embodiment of the present disclosure;

FIG. 9 is a cross-sectional view illustrating a display device according to an embodiment of the present disclosure; and

FIG. 10 is a cross-sectional view illustrating a display device according to an embodiment of the present disclosure.

### DETAILED DESCRIPTION

The subject matter of the present disclosure may be modified in various manners and have many forms, and thus specific embodiments will be illustrated in the drawings and described in more detail in the detailed description of the present disclosure. It should be understood, however, that it is not intended to limit the subject matter of the present disclosure to the particular forms disclosed, but rather, is intended to cover all modifications, equivalents, and alternatives falling within the spirit and scope of the present disclosure.

When explaining each of drawings, like reference numbers are used for referring to like elements. In the accompanying drawings, the dimensions of each structure may be exaggeratingly illustrated for clarity of the present disclosure. It will be understood that, although the terms “first,” “second,” etc., may be used herein to describe various components, these components should not be limited by these terms. These terms are only used to distinguish one component from another. For example, a first component could be termed a second component, and, similarly, a second component could be termed a first component, without departing from the scope of example embodiments of the present disclosure. The terms of a singular form may include plural forms unless the context clearly indicates otherwise.

In the present application, it will be understood that the terms “include,” “have” or the like specify the presence of features, numbers, steps, operations, components, parts, or combinations thereof disclosed in the specification, but do not exclude the possibility of presence or addition of one or more other features, numbers, steps, operations, components, parts, or combinations thereof.

In the present application, when a layer, a film, a region, or a plate is referred to as being “above” or “in an upper portion of” another layer, film, region, or plate, it can be not only directly on the layer, film, region, or plate, but intervening layers, films, regions, or plates may also be present. On the contrary to this, when a layer, a film, a region, or a plate is referred to as being “below,” or “in a lower portion of” another layer, film, region, or plate, it can be not only directly under the layer, film, region, or plate, but intervening layers, films, regions, or plates may also be present. In addition, it will be understood that when a part is referred to as being “on” another part, it can be above the other part, or under the other part as well.

In the specification, the term “substituted or unsubstituted” may mean substituted or unsubstituted with at least one substituent selected from the group consisting of a deuterium atom, a halogen atom, a cyano group, a nitro group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the substituents described above may be substituted or unsubstituted. For example, a biphenyl group may be interpreted as an aryl group or a phenyl group substituted with a phenyl group.

In the specification, the phrase “bonded to an adjacent group to form a ring” may mean that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In addition, the rings formed by being bonded to each other may be connected to another ring to form a spiro structure.

In the specification, the term “adjacent group” may mean a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In addition, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be a linear, branched or cyclic alkyl group. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments of the present disclosure are not limited thereto.

In the specification, a cycloalkyl group may mean a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments of the present disclosure are not limited thereto.

In the specification, an alkenyl group means a hydrocarbon group including at least one carbon double bond in the middle or terminal of an alkyl group having 2 or more carbon atoms. The alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, etc., but embodiments of the present disclosure are not limited thereto.

In the specification, an aryl group means any suitable functional group or substituent derived from an aromatic hydrocarbon ring. The aryl group may be a monocyclic aryl group or a polycyclic aryl group. The number of ring-forming carbon atoms in the aryl group may be 6 to 40, 6 to 30, 6 to 20, or 6 to 15. Examples of the aryl group may include a phenyl group, a naphthyl group, a fluorene group, an anthracene group, a phenanthrene group, a biphenyl group, a terphenyl group, a quaterphenyl group, a quinquephenyl group, a sexiphenyl group, a triphenylenyl group, a pyrenyl group, a benzofluoranthene group, a chrysene group, etc., but embodiments of the present disclosure are not limited thereto.

In the specification, the fluorenyl group may be substituted, and two substituents may be bonded to each other to form a spiro structure. Examples of the substituted fluorenyl group are as follows. However, embodiments of the present disclosure are not limited thereto.

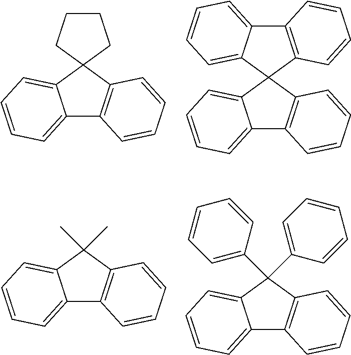

The heterocyclic group herein means any functional group or substituent derived from a ring containing at least one selected from B, O, N, P, Si, and Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the specification, the heteroaryl group may include at least one selected from B, O, N, P, Si, and S as a heteroatom. When the heteroaryl group contains two or more heteroatoms, the two or more heteroatoms may be the same as or different from each other. The heteroaryl group may be a monocyclic heterocyclic group or a polycyclic heterocyclic group. The ring-forming carbon number of the heteroaryl group may be 2 to 40, 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments of the present disclosure are not limited thereto.

In the specification, the above description of the aryl group may be applied to an arylene group except that the arylene group is a divalent group. The above description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but embodiments of the present disclosure are not limited thereto.

In the specification, the thio group may include an alkylthio group and an arylthio group. The thio group may mean that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments of the present disclosure are not limited thereto.

In the specification, an oxy group may mean that an oxygen atom is bonded to the alkyl group or the aryl group as defined above. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc., but embodiments of the present disclosure are not limited thereto.

The boron group herein may mean that a boron atom is bonded to the alkyl group or the aryl group as defined above. The boron group includes an alkyl boron group and an aryl boron group. Examples of the boron group may include a dimethylboron group, a trimethylboron group, a t-butyldimethylboron group, a diphenylboron group, a phenylboron group, etc., but embodiments of the present disclosure are not limited thereto.

In the specification, the number of carbon atoms in an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, etc., but embodiments of the present disclosure are not limited thereto.

In the specification, a direct linkage may mean a single bond.

In the specification,

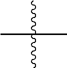

and “-*” mean a position to be connected.

Hereinafter, embodiments of the present disclosure will be described with reference to the accompanying drawings.

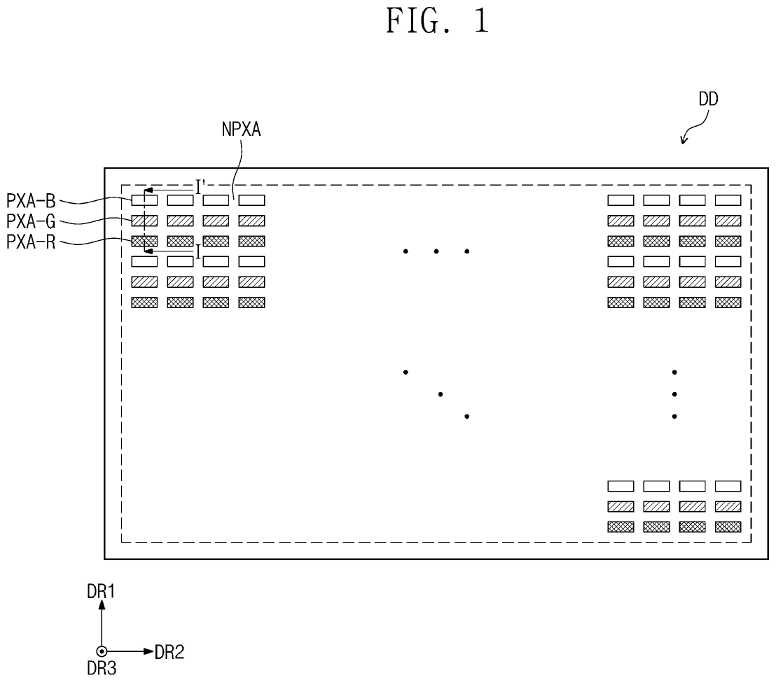

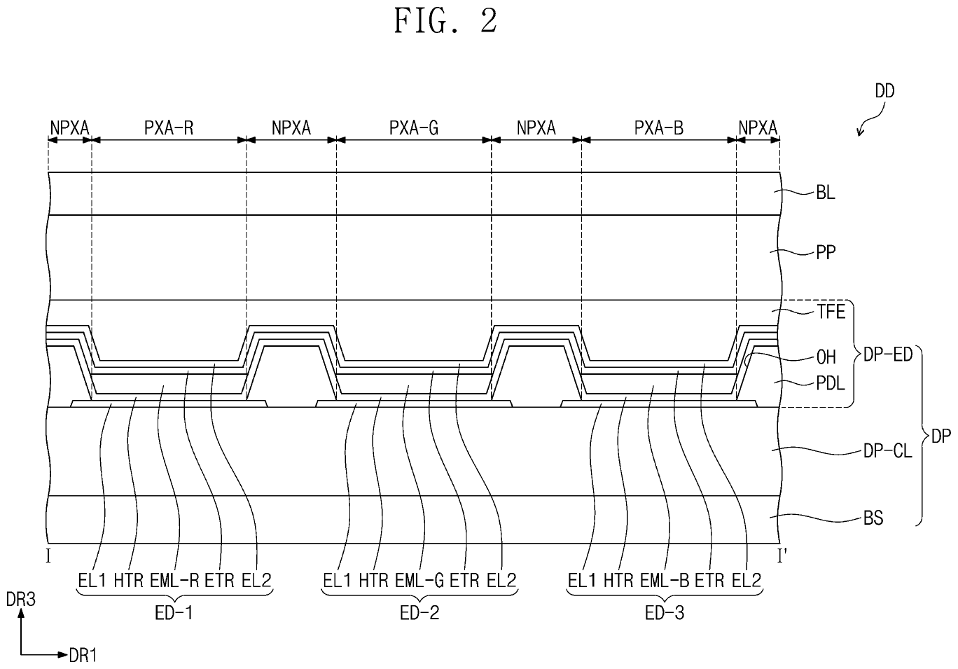

FIG. 1 is a plan view illustrating an embodiment of a display device DD. FIG. 2 is a cross-sectional view of the display device DD of the embodiment. FIG. 2 is a cross-sectional view illustrating a part taken along line I-I′ of FIG. 1.

The display device DD may include a display panel DP and an optical layer PP on the display panel DP. The display panel DP includes light emitting elements ED-1, ED-2, and ED-3. The display device DD may include a plurality of light emitting elements ED-1, ED-2, and ED-3. The optical layer PP may be on the display panel DP to control reflected light in the display panel DP due to external light. The optical layer PP may include, for example, a polarization layer and/or a color filter layer. In some embodiments, the optical layer PP may be omitted from the display device DD of an embodiment.

A base substrate BL may be on the optical layer PP. The base substrate BL may be a member which provides a base surface that the optical layer PP is on. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may be omitted.

The display device DD according to an embodiment may further include a filling layer. The filling layer may be between a display element layer DP-ED and the base substrate BL. The filling layer may be an organic material layer. The filling layer may include at least one selected from of an acrylic-based resin, a silicone-based resin, and an epoxy-based resin.

The display panel DP may include a base layer BS, a circuit layer DP-CL provided on the base layer BS, and the display element layer DP-ED. The display element layer DP-ED may include a pixel defining film PDL, the light emitting elements ED-1, ED-2, and ED-3 between portions of the pixel defining film PDL, and an encapsulation layer TFE on the light emitting elements ED-1, ED-2, and ED-3.

The base layer BS may be a member which provides a base surface that the display element layer DP-ED is on. The base layer BS may be a glass substrate, a metal substrate, a plastic substrate, etc. However, the embodiment is not limited thereto, and the base layer BS may be an inorganic layer, an organic layer, or a composite material layer.

In an embodiment, the circuit layer DP-CL is on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display element layer DP-ED.

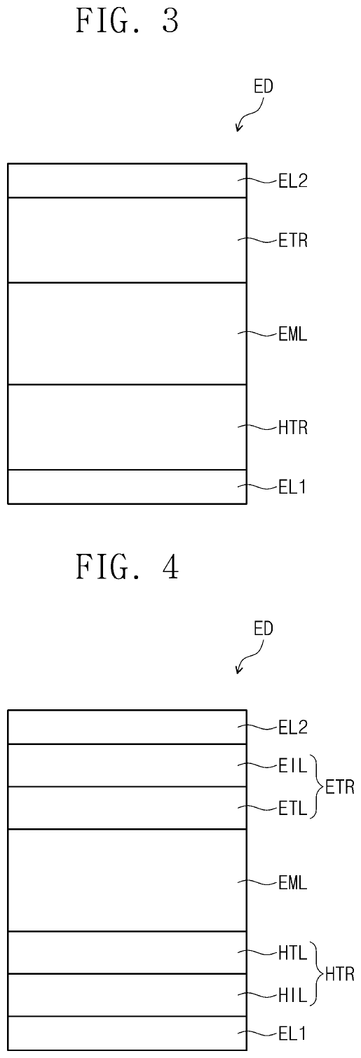

Each of the light emitting elements ED-1, ED-2, and ED-3 may have a structure of each light emitting element ED of embodiments according to FIGS. 3 to 6, which will be further described herein below. Each of the light emitting elements ED-1, ED-2, and ED-3 may include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light emitting elements ED-1, ED-2, and ED-3 are in openings OH defined in the pixel defining film PDL, and the hole transport region HTR, the electron transport region ETR, and the second electrode EL2 are provided as a common layer in the entire light emitting elements ED-1, ED-2, and ED-3. However, embodiments of the present disclosure are not limited thereto, and, in some embodiments, the hole transport region HTR and the electron transport region ETR in an embodiment may be provided by being patterned inside the openings OH defined in the pixel defining film PDL. For example, the hole transport region HTR, the emission layers EML-R, EML-G, and EML-B, and the electron transport region ETR of the light emitting elements ED-1, ED-2, and ED-3 in an embodiment may be provided by being patterned in an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects the display element layer DP-ED from moisture/oxygen, and the encapsulation-organic film protects the display element layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. The encapsulation-organic film may include an acrylic-based compound, an epoxy-based compound, and/or the like. The encapsulation-organic film may include a photopolymerizable organic material, but embodiments of the present disclosure are not particularly limited thereto.

The encapsulation layer TFE may be on the second electrode EL2 and may fill the opening OH.

Referring to FIGS. 1 and 2, the display device DD may include a non-light emitting region NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced apart from each other on a plane.

Each of the light emitting regions PXA-R, PXA-G, and PXA-B may be a region divided by the pixel defining film PDL. The non-light emitting areas NPXA may be areas between the adjacent light emitting areas PXA-R, PXA-G, and PXA-B, which correspond to the pixel defining film PDL. In the specification, the light emitting regions PXA-R, PXA-G, and PXA-B may respectively correspond to pixels. The pixel defining film PDL may divide the light emitting elements ED-1, ED-2, and ED-3. The emission layers EML-R, EML-G, and EML-B of the light emitting elements ED-1, ED-2, and ED-3 may be in openings OH defined in the pixel defining film PDL and separated (e.g., spaced apart) from each other.

The light emitting regions PXA-R, PXA-G, and PXA-B may be divided into a plurality of groups according to the color of light generated from the light emitting elements ED-1, ED-2, and ED-3. In the display device DD of an embodiment illustrated in FIGS. 1 and 2, three light emitting regions PXA-R, PXA-G, and PXA-B, which emit red light, green light, and blue light, respectively, are illustrated as examples. For example, the display device DD of an embodiment may include the red light emitting region PXA-R, the green light emitting region PXA-G, and the blue light emitting region PXA-B that are separated (e.g., spaced apart) from each other.

In the display device DD according to an embodiment, the plurality of light emitting elements ED-1, ED-2 and ED-3 may emit light beams having wavelengths different from each other. For example, in an embodiment, the display device DD may include a first light emitting element ED-1 that emits red light, a second light emitting element ED-2 that emits green light, and a third light emitting element ED-3 that emits blue light. In some embodiments, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, embodiments of the present disclosure are not limited thereto, and the first to third light emitting elements ED-1, ED-2, and ED-3 may emit light beams in the same wavelength range or at least one light emitting element may emit a light beam in a wavelength range different from the others. For example, the first to third light emitting elements ED-1, ED-2, and ED-3 may all emit blue light.

The light emitting regions PXA-R, PXA-G, and PXA-B in the display device DD according to an embodiment may be arranged in a stripe form. Referring to FIG. 1, the plurality of red light emitting regions PXA-R, the plurality of green light emitting regions PXA-G, and the plurality of blue light emitting regions PXA-B each may be arranged along a second directional axis DR2. In addition, the red light emitting region PXA-R, the green light emitting region PXA-G, and the blue light emitting region PXA-B may be alternately arranged in this order along a first directional axis DR1.

FIGS. 1 and 2 illustrate that all the light emitting regions PXA-R, PXA-G, and PXA-B have similar area, but embodiments of the present disclosure are not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may mean areas when viewed on a plane defined by the first directional axis DR1 and the second directional axis DR2.

An arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in various suitable combinations according to the characteristics of display quality required or desired in the display device DD. For example, the arrangement form of the light emitting regions PXA-R, PXA-G, and PXA-B may be a PENTILE® arrangement form (e.g., an RGBG matrix, RGBG structure, or RGBG matrix structure) or a diamond (Diamond Pixel™) arrangement form. PENTILE® is a duly registered trademark of Samsung Display Co., Ltd.

In addition, the areas of the light emitting regions PXA-R, PXA-G, and PXA-B may be different from each other. For example, in an embodiment, the area of the green light emitting region PXA-G may be smaller than that of the blue light emitting region PXA-B, but embodiments of the present disclosure are not limited thereto.

Hereinafter, FIGS. 3 to 6 are cross-sectional views schematically illustrating light emitting elements according to embodiments. The light emitting elements ED according to embodiments each may include a first electrode EL1, a second electrode EL2 facing the first electrode EL1, and at least one functional layer between the first electrode EL1 and the second electrode EL2. Each of the light emitting elements ED of embodiments may include an amine compound of an embodiment, which will be further described below, in at least one functional layer.

Each of the light emitting elements ED may include, as at least one functional layer, a hole transport region HTR, an emission layer EML, and an electron transport region ETR that are sequentially stacked. Referring to FIG. 3, the light emitting element ED of an embodiment may include a first electrode EL1, a hole transport region HTR, an emission layer EML, an electron transport region ETR, and a second electrode EL2 that are sequentially stacked.

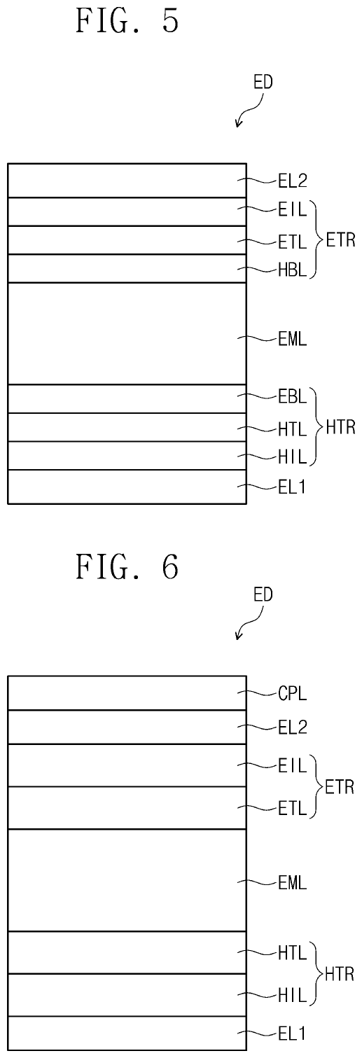

Compared with FIG. 3, FIG. 4 illustrates a cross-sectional view of a light emitting element ED of an embodiment, in which a hole transport region HTR includes a hole injection layer HIL and a hole transport layer HTL, and an electron transport region ETR includes an electron injection layer EIL and an electron transport layer ETL. In addition, compared with FIG. 3, FIG. 5 illustrates a cross-sectional view of a light emitting element ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting element ED of an embodiment including a capping layer CPL on a second electrode EL2.

The light emitting element ED of an embodiment may include an amine compound of an embodiment, which will be further described below, in the hole transport region HTR. In the light emitting element ED of an embodiment, at least one selected from a hole injection layer HIL, a hole transport layer HTL, and an electron blocking layer EBL in the hole transport region HTR may include the amine compound of an embodiment. For example, the hole transport layer HTL in the light emitting element ED of an embodiment may include an amine compound of an embodiment.

In the light emitting element ED according to an embodiment, the first electrode EL1 has conductivity (e.g., electrical conductivity). The first electrode EL1 may be formed of a metal material, a metal alloy, and/or a conductive compound (e.g., an electrically conductive compound). The first electrode EL1 may be an anode or a cathode. However, embodiments of the present disclosure are not limited thereto. In addition, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected from among these, a mixture of two or more selected from among these, and/or an oxide thereof.

If the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, and/or a compound and/or mixture thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but embodiments of the present disclosure are not limited thereto. In addition, embodiments of the present disclosure are not limited thereto, and the first electrode EL1 may include the above-described metal materials, combinations of at least two metal materials of the above-described metal materials, oxides of the above-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may have a single layer formed of a single material, a single layer formed of a plurality of different materials, or a multilayer structure including a plurality of layers formed of a plurality of different materials.

The hole transport region HTR may include at least one selected from the hole injection layer HIL, the hole transport layer HTL, and the electron blocking layer EBL. In some embodiments, the hole transport region HTR may include a plurality of stacked hole transport layers.

In some embodiments, the hole transport region HTR may have a single layer structure of the hole injection layer HIL or the hole transport layer HTL, or may have a single layer structure formed of a hole injection material and a hole transport material. In an embodiment, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, or a hole transport layer HTL/buffer layer are stacked in order from the first electrode EL1, but embodiments of the present disclosure are not limited thereto.

The thickness of the hole transport region HTR may be, for example, from about 50 Å to about 15,000 Å. The hole transport region HTR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of an embodiment may include the amine compound of an embodiment in the hole transport region HTR. In the light emitting element ED of an embodiment, the hole transport region HTR may include an electron injection layer EIL or a hole transport layer HTL, and the hole transport layer HTL may include the amine compound of an embodiment. The amine compound of an embodiment may be included in a layer, which is adjacent to the emission layer EML, among the layers included in the hole transport region HTR.

The amine compound of an embodiment includes a structure in which a first substituent, a second substituent, and a third substituent are linked to a core nitrogen atom. The amine compound of an embodiment may include an amine group, for example, the core nitrogen atom, and the first to third substituents may be bonded to the core nitrogen atom of the amine compound of an embodiment. In an embodiment, each of the first substituent and the second substituent necessarily includes a dibenzoheterol moiety. In the present specification, the “dibenzoheterol” moiety means any one selected from among a dibenzofuran moiety and a dibenzothiophene moiety.

The first substituent is directly bonded to the core nitrogen atom at the fourth carbon position, and an aryl group or a heteroaryl group is necessarily substituted at the first or third carbon position of the first substituent. In some embodiments, a carbazole moiety is not linked at the first or third carbon position of the first substituent. The second substituent is linked to the core nitrogen atom via an arylene linker or a heteroarylene linker, or directly linked to the core nitrogen atom without a separate linker. The third substituent is an aryl group or a heteroaryl group directly linked to the core nitrogen atom.

In some embodiments, the amine compound of an embodiment may not include a fluorene moiety, a benzonaphthofuran moiety, a benzonaphthothiophene moiety, and a benzobisbenzofuran moiety in the molecular structure. For the amine compound of an embodiment, the case where each of the first to third substituents is a fluorene moiety, a benzonaphthofuran moiety, a benzonaphthothiophene moiety, or a benzobisbenzofuran moiety may be excluded, and the case where a substituent substituted at each of the first to third substituents is a fluorene moiety, a benzonaphthofuran moiety, a benzonaphthothiophene moiety, or a benzobisbenzofuran moiety may also be excluded.

In the present specification, the numbers of carbon atoms of the dibenzoheterol moiety are assigned as represented by Formula S1 below:

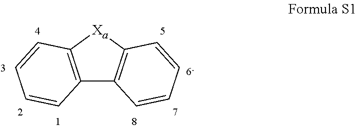

With respect to the carbon numbering of the first substituent, in the case where the first substituent is located such that Xₐ is on the top of the first substituent like Formula S1 above, the numbers are assigned in a counterclockwise direction from the carbon atom, at the ortho-position with Xₐ, from among the carbon atoms constituting the left benzene ring, and the carbon number at the condensation position is excluded. For convenience of description, substituents linked to benzene rings at both sides in Formula S1 above are omitted. Unlike Formula S1, the first substituent may have at least one substituent in addition to hydrogen atoms. However, embodiments of the present disclosure are not limited thereto.

In Formula S1, Xₐ is O or S. In Formula S1, when Xₐ is 0, the first substituent may be a substituted or unsubstituted dibenzofuran group. In Formula S1, when Xₐ is S, the first substituent may be a substituted or unsubstituted dibenzothiophene group.

The amine compound of an embodiment may be a monoamine compound including a single amine group. The amine compound of an embodiment may be a monoamine compound having a single amine group which does not form a ring in the molecular structure. The amine compound of an embodiment may be a compound including a single nitrogen atom in the molecular structure.

In an embodiment, the amine compound may be represented by Formula 1 below:

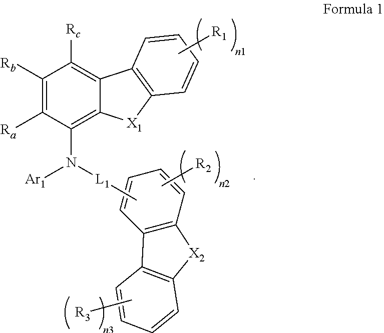

In Formula 1, X₁ and X₂ are each independently O or S. X₁ and X₂ may be the same as or different from each other. In an embodiment, both X₁ and X₂ may be O or both may be S. In some embodiments, one selected from among X₁ and X₂ may be 0, and the other may be S.

In Formula 1, Ar₁ is a substituted or unsubstituted aryl group having 6 to 40 ring-forming carbon atoms, or a substituted or unsubstituted heteroaryl group having 2 to 40 ring-forming carbon atoms. Ar₁ may be a substituent corresponding to the above-described third substituent. In an embodiment, Ar₁ may be a substituted or unsubstituted phenyl group, a substituted or unsubstituted biphenyl group, a substituted or unsubstituted terphenyl group, a substituted or unsubstituted naphthyl group, a substituted or unsubstituted phenanthrene group, a substituted or unsubstituted dibenzofuranyl group, or a substituted or unsubstituted dibenzothiophenyl group. In an embodiment, Ar₁ may be an unsubstituted phenyl group, an unsubstituted biphenyl group, an unsubstituted terphenyl group, an unsubstituted naphthyl group, an unsubstituted phenanthrene group, an unsubstituted dibenzofuranyl group, or an unsubstituted dibenzothiophenyl group. If Ar₁ is substituted, the substituent may be an unsubstituted phenyl group, an unsubstituted biphenyl group, an unsubstituted naphthyl group, or an unsubstituted dibenzofuranyl group. In some embodiments, in the amine compound of an embodiment, the case where Ar₁ includes a fluorenyl group, a benzonaphthofuranyl group, a benzonaphthothiophenyl group, and a benzobisbenzofuranyl group is excluded. For example, in the amine compound of an embodiment, the case where Ar₁ is a fluorenyl group, a benzonaphthofuranyl group, a benzonaphthothiophenyl group, or a benzobisbenzofuranyl group is excluded, and the case where Ar₁ includes a fluorenyl group, a benzonaphthofuranyl group, a benzonaphthothiophenyl group, or a benzobisbenzofuranyl group as a substituent is excluded. In some embodiments, the amine compound of an embodiment may exclude the case where Ar₁ is bonded to an adjacent group to form a ring.

In Formula 1, Rₐ to R_{c} may be each independently a hydrogen atom, a deuterium atom, a substituted or unsubstituted aryl group having 6 to 40 ring-forming carbon atoms, or a substituted or unsubstituted heteroaryl group having 2 to 40 ring-forming carbon atoms. The dibenzoheterol moiety substituted with Rₐ to R_{c} may be a substituent corresponding to the above-described first substituent. In an embodiment, R_{b} may be a hydrogen atom or a deuterium atom.

At least one selected from Rₐ and R_{c} is a substituted or unsubstituted aryl group having 6 to 40 ring-forming carbon atoms, or a substituted or unsubstituted heteroaryl group having 2 to 40 ring-forming carbon atoms, At least one selected from Rₐ and R_{c} may be a substituted or unsubstituted phenyl group, a substituted or unsubstituted naphthyl group, a substituted or unsubstituted biphenyl group, a substituted or unsubstituted phenanthrenyl group, a substituted or unsubstituted dibenzofuranyl group, or a substituted or unsubstituted dibenzothiophenyl group. If each of Rₐ and R_{c} is substituted, the substituent may be an unsubstituted phenyl group, an unsubstituted biphenyl group, an unsubstituted naphthyl group, or an unsubstituted dibenzofuranyl group. In some embodiments, the amine compound of an embodiment may exclude the case where each of Rₐ to R_{c} is bonded to an adjacent group to form a ring.

In some embodiments, in the amine compound of an embodiment, the case where each of Rₐ to R_{c} includes a carbazole group, a fluorenyl group, a benzonaphthofuranyl group, a benzonaphthothiophenyl group, or a benzobisbenzofuranyl group is excluded. For example, in the amine compound of an embodiment, the case where each of Rₐ to R_{c} is a carbazole group, a fluorenyl group, a benzonaphthofuranyl group, a benzonaphthothiophenyl group, or a benzobisbenzofuranyl group is excluded, and the case where each of Rₐ to R_{c} includes a carbazole group, a fluorenyl group, a benzonaphthofuranyl group, a benzonaphthothiophenyl group, or a benzobisbenzofuranyl group as a substituent is excluded.

In Formula 1, R₁ to R₃ are each independently a hydrogen atom, a deuterium atom, a halogen atom, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms. In an embodiment, R₁ to R₃ may be each independently a hydrogen atom, a deuterium atom, or a substituted or unsubstituted phenyl group. R₁ may be a hydrogen atom or a deuterium atom, and R₂ and R₃ may be each independently a hydrogen atom, a deuterium atom, or an unsubstituted phenyl group. The dibenzoheterol moiety substituted with Rₐ to R_{c} may be a substituent corresponding to the above-described second substituent. In some embodiments, the amine compound of an embodiment may exclude the case where each of R₁ to R₃ is bonded to an adjacent group to form a ring.

In Formula 1, L₁ is a direct linkage, a substituted or unsubstituted arylene group having 6 to 40 ring-forming carbon atoms, or a substituted or unsubstituted heteroarylene group having 2 to 40 ring-forming carbon atoms. In an embodiment, L₁ may be a direct linkage, or a substituted or unsubstituted p-phenylene group. L₁ may be a direct linkage, or an unsubstituted p-phenylene group. In some embodiments, in the amine compound of an embodiment, the case where L₁ includes a fluorenyl group, a benzonaphthofuranyl group, a benzonaphthothiophenyl group, and a benzobisbenzofuranyl group is excluded. For example, in the amine compound of an embodiment, the case where L₁ is a divalent fluorenyl group, a divalent benzonaphthofuranyl group, a divalent benzonaphthothiophenyl group, or a divalent benzobisbenzofuranyl group is excluded, and the case where L₁ includes a fluorenyl group, a benzonaphthofuranyl group, a benzonaphthothiophenyl group, or a benzobisbenzofuranyl group as a substituent is excluded. In some embodiments, the amine compound of an embodiment may exclude the case where L₁ is bonded to an adjacent group to form a ring.

In Formula 1, n1 and n3 are each independently an integer of 0 to 4. N2 is an integer of 0 to 3. The case where n1 is 0 may mean that the amine compound of an embodiment is not substituted with R₁. The case where n1 is 4 and each of R₁'s is a hydrogen atom may be the same as the case where n1 is 0. If n1 is an integer of 2 or more, a plurality of R₁'s may each be the same, or at least one selected from among the plurality of R₁'s may be different from the others. The case where n2 is 0 may mean that the amine compound of a mean embodiment is not substituted with R₂. The case where n2 is 3 and each of R₂'s is a hydrogen atom may be the same as the case where n2 is 0. If n2 is an integer of 2 or more, a plurality of R₂'s may each be the same, or at least one selected from among the plurality of R₂'s may be different from the others. The case where n3 is 0 may mean that the amine compound of a mean embodiment is not substituted with R₃. The case where n3 is 4 and each of R₃'s is a hydrogen atom may be the same as the case where n3 is 0. If n3 is an integer of 2 or more, a plurality of R₃'s may each be the same, or at least one selected from among the plurality of R₃'s may be different from the others.

In an embodiment, the amine compound may be represented by any one selected from among Formula 2-1 to Formula 2-3 below:

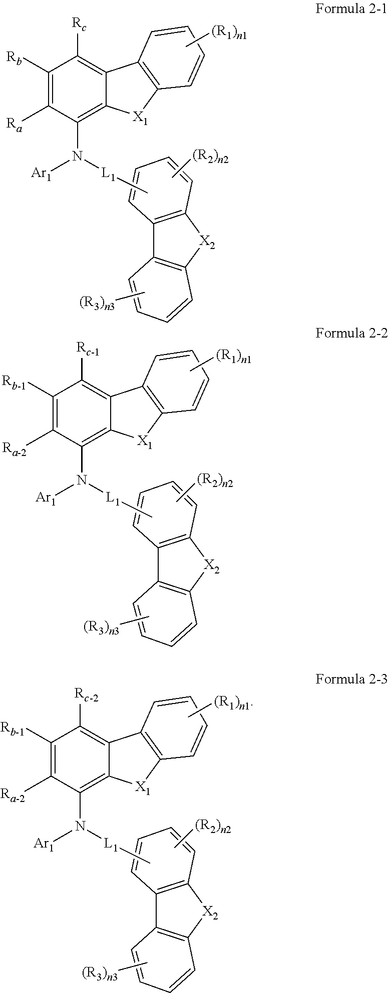

Formula 2-1 to Formula 2-3 represent the cases where the types of Rₐ to R_{c} in Formula 1 are specified. Formula 2-1 represents the case where in Formula 1, the position of Re is specified with an aryl group or a heteroaryl group, and the positions of Rₐ and R_{b} are hydrogen atoms or deuterium atoms. Formula 2-2 represents the case where in Formula 1, the position of Rₐ is specified with an aryl group or a heteroaryl group, and the positions of R_{b} and R_{c} are hydrogen atoms or deuterium atoms. Formula 2-3 represents the case where in Formula 1, the positions of Rₐ and R_{c} are specified with an aryl group or a heteroaryl group, and the position of R_{b} is a hydrogen atom or a deuterium atom.

In Formula 2-1 to Formula 2-3, Rₐ₋₁, R_{b-1}, and R_{c-1} are each independently a hydrogen atom or a deuterium atom. Rₐ₋₂ and R_{c-2} are each independently a substituted or unsubstituted aryl group having 6 to 40 ring-forming carbon atoms, or a substituted or unsubstituted heteroaryl group having 2 to 40 ring-forming carbon atoms, In an embodiment, Rₐ₋₂ and R_{c-2} may be each independently a substituted or unsubstituted phenyl group, a substituted or unsubstituted naphthyl group, a substituted or unsubstituted biphenyl group, a substituted or unsubstituted phenanthrenyl group, a substituted or unsubstituted dibenzofuranyl group, or a substituted or unsubstituted dibenzothiophenyl group. If each of Rₐ₋₂ and R_{c-2} is substituted, the substituent may be an unsubstituted phenyl group, an unsubstituted biphenyl group, an unsubstituted naphthyl group, or an unsubstituted dibenzofuranyl group.

In some embodiments, in the amine compound of an embodiment, the case where each of Rₐ₋₂ and R_{c-2} includes a carbazole group, a fluorenyl group, a benzonaphthofuranyl group, a benzonaphthothiophenyl group, or a benzobisbenzofuranyl group is excluded. For example, in the amine compound of an embodiment, the case where each of Rₐ₋₂ and R_{c-2} is a carbazole group, a fluorenyl group, a benzonaphthofuranyl group, a benzonaphthothiophenyl group, or a benzobisbenzofuranyl group is excluded, and the case where each of Rₐ₋₂ and R_{c-2} includes a carbazole group, a fluorenyl group, a benzonaphthofuranyl group, a benzonaphthothiophenyl group, or a benzobisbenzofuranyl group as a substituent is excluded.

In Formula 2-1 to Formula 2-3 above, the same as described with respect to Formula 1 above may be applied to X₁, X₂, Ar₁, R₁ to R₃, L₁, n1, n2, and n3.

In an embodiment, the amine compound represented by Formula 1 may be represented by any one selected from among Formula 3-1 to Formula 3-4 below:

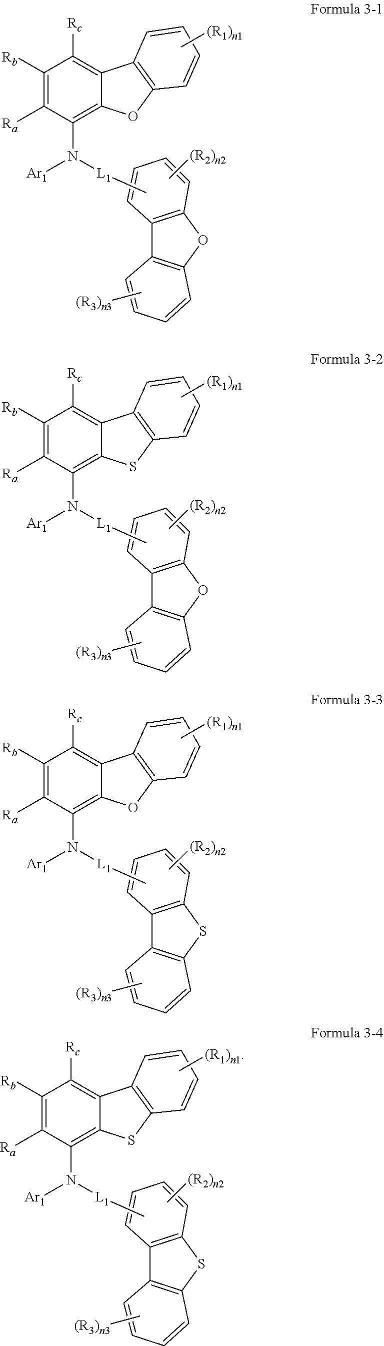

Formula 3-1 to Formula 3-4 represent the cases where in Formula 1, X₁ and X₂ are specified as O or S. Formula 3-1 represents the case where in Formula 1, both X₁ and X₂ are O, Formula 3-2 represents the case where in Formula 1, X₁ is S and X₂ is O, Formula 3-3 represents the case where in Formula 1, X₁ is O and X₂ is S, and Formula 3-4 represents the case where in Formula 1, both X₁ and X₂ are S.

In Formula 3-1 to Formula 3-4, the same as described with respect to Formula 1 above may be applied to Ar₁, Rₐ to R_{c}, R₁ to R₃, L₁, n1, n2, and n3.

In an embodiment, the amine compound represented by Formula 1 may be represented by any one selected from among Formula 4-1 to Formula 4-4 below:

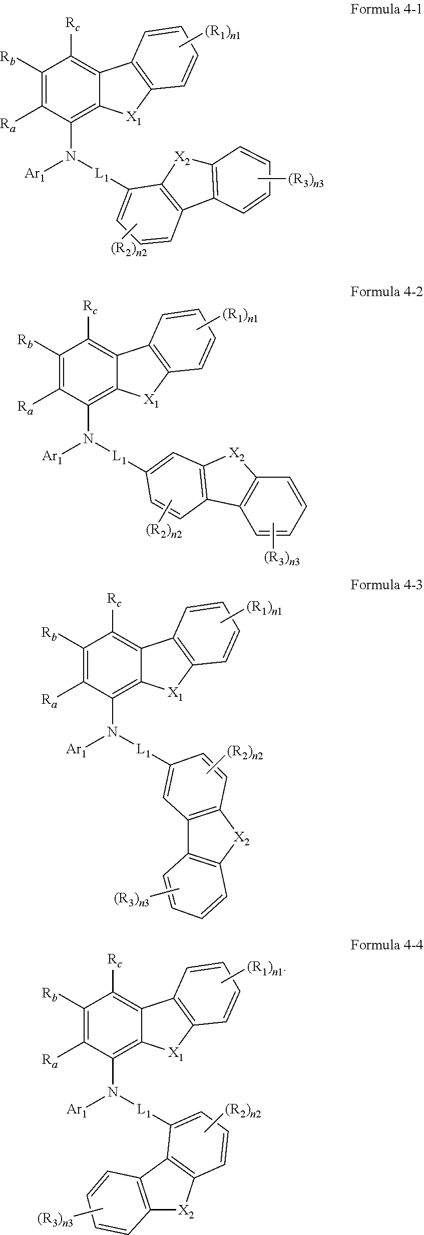

Formula 4-1 to Formula 4-4 represent the cases where in Formula 1, the position in which the dibenzoheterol moiety corresponding to the above-described second substituent is linked to L₁ linker, is specified. Formula 4-1 is the case where the dibenzoheterol moiety is linked to the L₁ linker at carbon 1 of the dibenzoheterol moiety corresponding to the second substituent, Formula 4-2 is the case where the case where the dibenzoheterol moiety is linked to the L₁ linker at carbon 2 of the dibenzoheterol moiety corresponding to the second substituent, Formula 4-3 is the case where the dibenzoheterol moiety is linked to the L₁ linker at carbon 3 of the dibenzoheterol moiety corresponding to the second substituent, and Formula 4-4 is the case where the dibenzoheterol moiety is linked to the L₁ linker at carbon 4 of the dibenzoheterol moiety corresponding to the second substituent.

In Formula 4-1 to Formula 4-4, the same as described with respect to Formula 1 above may be applied to X₁, X₂, Ar₁, Rₐ to R_{c}, R₁ to R₃, L₁, n1, n2, and n3.

The amine compound of an embodiment may be represented by one selected from among the compounds in Compound Group 1 below. The hole transport region HTR of the light emitting element ED of an embodiment may include at least one selected from among the amine compounds disclosed in Compound Group 1 below. For example, the hole transport layer HTL of the light emitting element ED may include at least one selected from among the amine compounds disclosed in Compound Group 1 below:

### Compound Group 1

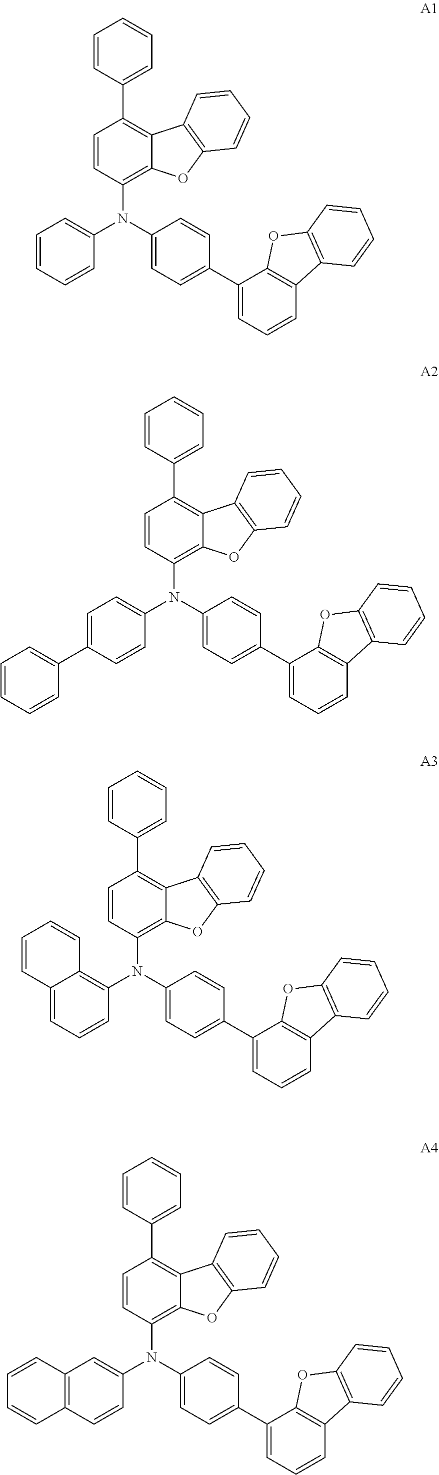 [SMILES: c1ccc(-c2ccc(N(c3ccccc3)c3ccc(-c4cccc5c4oc4ccccc45)cc3)c3oc4ccccc4c23)cc1 ; c1ccc(-c2ccc(N(c3ccc(-c4cccc5c4oc4ccccc45)cc3)c3ccc(-c4ccccc4)c4c3oc3ccccc34)cc2)cc1 ; c1ccc(-c2ccc(N(c3ccc(-c4cccc5c4oc4ccccc45)cc3)c3cccc4ccccc34)c3oc4ccccc4c23)cc1 ; c1ccc(-c2ccc(N(c3ccc(-c4cccc5c4oc4ccccc45)cc3)c3ccc4ccccc4c3)c3oc4ccccc4c23)cc1]

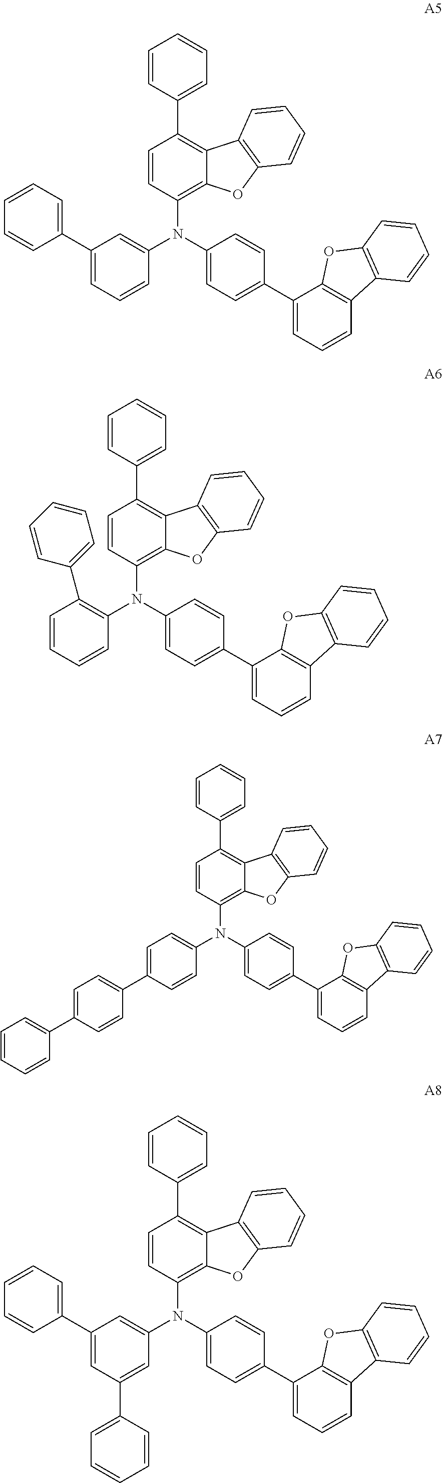 [SMILES: c1ccc(-c2cccc(N(c3ccc(-c4cccc5c4oc4ccccc45)cc3)c3ccc(-c4ccccc4)c4c3oc3ccccc34)c2)cc1 ; c1ccc(-c2ccccc2N(c2ccc(-c3cccc4c3oc3ccccc34)cc2)c2ccc(-c3ccccc3)c3c2oc2ccccc23)cc1 ; c1ccc(-c2ccc(-c3ccc(N(c4ccc(-c5cccc6c5oc5ccccc56)cc4)c4ccc(-c5ccccc5)c5c4oc4ccccc45)cc3)cc2)cc1 ; c1ccc(-c2cc(-c3ccccc3)cc(N(c3ccc(-c4cccc5c4oc4ccccc45)cc3)c3ccc(-c4ccccc4)c4c3oc3ccccc34)c2)cc1]

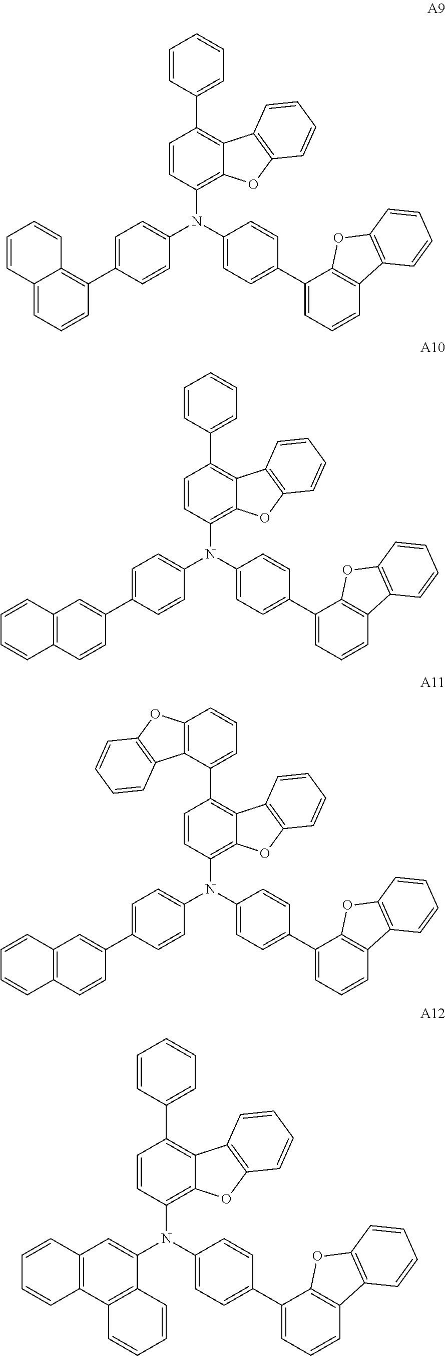 [SMILES: c1ccc(-c2ccc(N(c3ccc(-c4cccc5ccccc45)cc3)c3ccc(-c4cccc5c4oc4ccccc45)cc3)c3oc4ccccc4c23)cc1 ; c1ccc(-c2ccc(N(c3ccc(-c4ccc5ccccc5c4)cc3)c3ccc(-c4cccc5c4oc4ccccc45)cc3)c3oc4ccccc4c23)cc1 ; c1ccc2cc(-c3ccc(N(c4ccc(-c5cccc6c5oc5ccccc56)cc4)c4ccc(-c5cccc6oc7ccccc7c56)c5c4oc4ccccc45)cc3)ccc2c1 ; c1ccc(-c2ccc(N(c3ccc(-c4cccc5c4oc4ccccc45)cc3)c3cc4ccccc4c4ccccc34)c3oc4ccccc4c23)cc1]

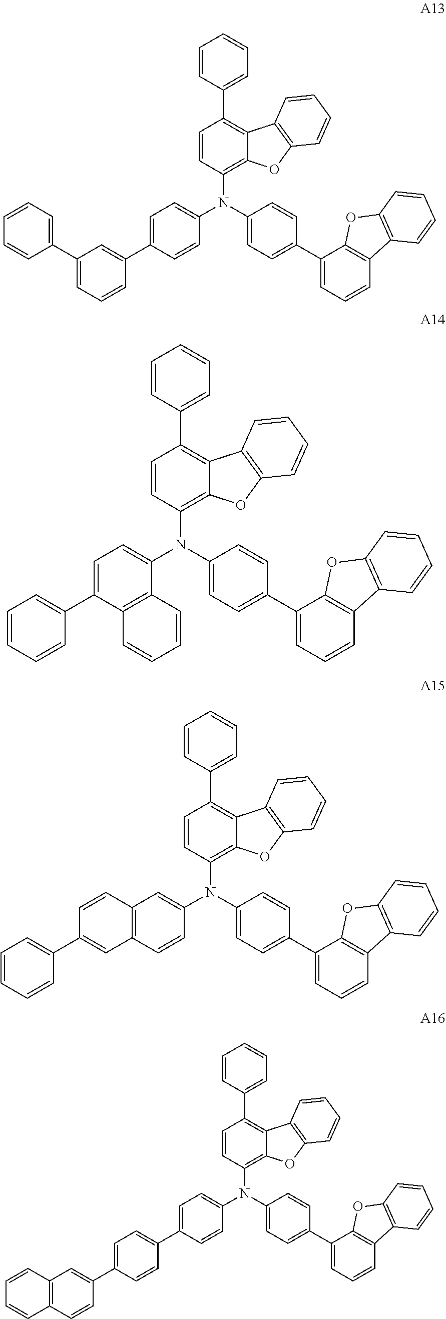 [SMILES: c1ccc(-c2cccc(-c3ccc(N(c4ccc(-c5cccc6c5oc5ccccc56)cc4)c4ccc(-c5ccccc5)c5c4oc4ccccc45)cc3)c2)cc1 ; c1ccc(-c2ccc(N(c3ccc(-c4cccc5c4oc4ccccc45)cc3)c3ccc(-c4ccccc4)c4c3oc3ccccc34)c3ccccc23)cc1 ; c1ccc(-c2ccc3cc(N(c4ccc(-c5cccc6c5oc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4cccc5c4oc4ccccc45)cc3)c3oc4ccccc4c23)cc1]

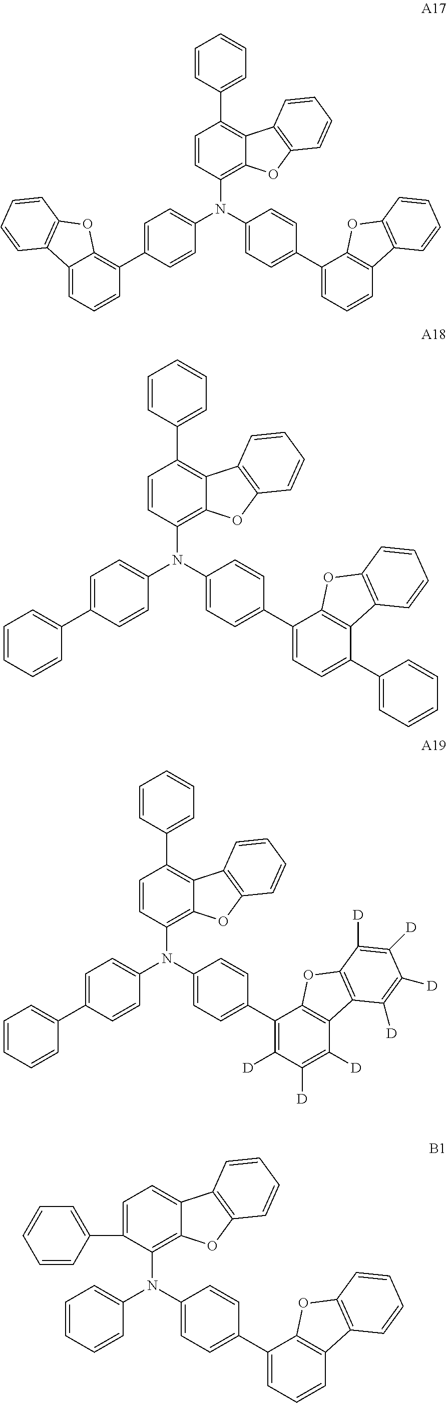 [SMILES: c1ccc(-c2ccc(N(c3ccc(-c4cccc5c4oc4ccccc45)cc3)c3ccc(-c4cccc5c4oc4ccccc45)cc3)c3oc4ccccc4c23)cc1 ; c1ccc(-c2ccc(N(c3ccc(-c4ccc(-c5ccccc5)c5c4oc4ccccc45)cc3)c3ccc(-c4ccccc4)c4c3oc3ccccc34)cc2)cc1 ; [2H]c1c([2H])c([2H])c2c(oc3c(-c4ccc(N(c5ccc(-c6ccccc6)cc5)c5ccc(-c6ccccc6)c6c5oc5ccccc56)cc4)c([2H])c([2H])c([2H])c32)c1[2H] ; c1ccc(-c2ccc3c(oc4ccccc43)c2N(c2ccccc2)c2ccc(-c3cccc4c3oc3ccccc34)cc2)cc1]

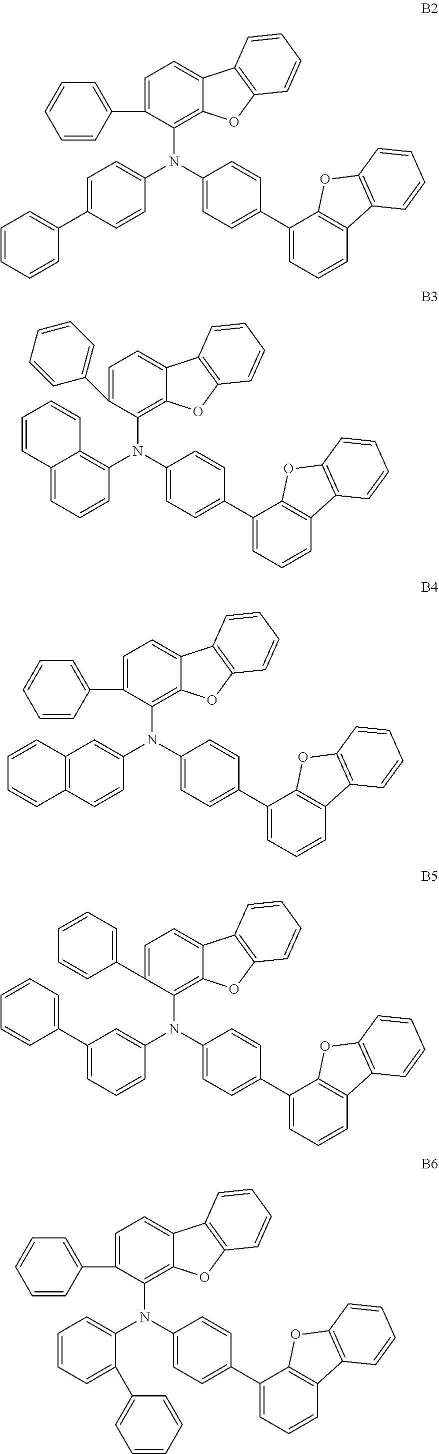 [SMILES: c1ccc(-c2ccc(N(c3ccc(-c4cccc5c4oc4ccccc45)cc3)c3c(-c4ccccc4)ccc4c3oc3ccccc34)cc2)cc1 ; c1ccc(-c2ccc3c(oc4ccccc43)c2N(c2ccc(-c3cccc4c3oc3ccccc34)cc2)c2cccc3ccccc23)cc1 ; c1ccc(-c2ccc3c(oc4ccccc43)c2N(c2ccc(-c3cccc4c3oc3ccccc34)cc2)c2ccc3ccccc3c2)cc1 ; c1ccc(-c2cccc(N(c3ccc(-c4cccc5c4oc4ccccc45)cc3)c3c(-c4ccccc4)ccc4c3oc3ccccc34)c2)cc1 ; c1ccc(-c2ccccc2N(c2ccc(-c3cccc4c3oc3ccccc34)cc2)c2c(-c3ccccc3)ccc3c2oc2ccccc23)cc1]

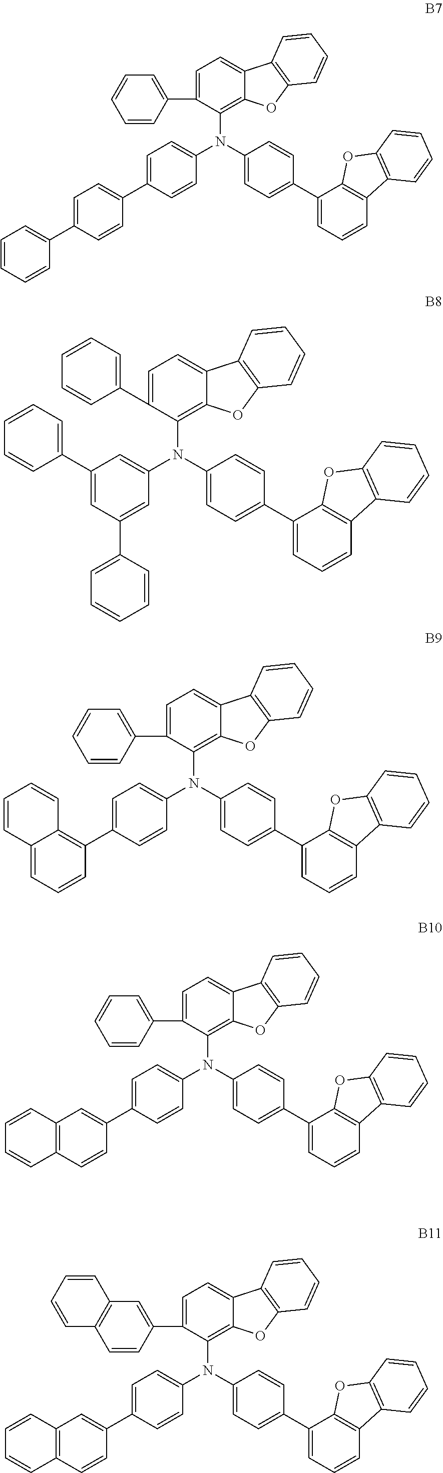 [SMILES: c1ccc(-c2ccc(-c3ccc(N(c4ccc(-c5cccc6c5oc5ccccc56)cc4)c4c(-c5ccccc5)ccc5c4oc4ccccc45)cc3)cc2)cc1 ; c1ccc(-c2cc(-c3ccccc3)cc(N(c3ccc(-c4cccc5c4oc4ccccc45)cc3)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc(-c3cccc4c3oc3ccccc34)cc2)cc1 ; c1ccc(-c2ccc3c(oc4ccccc43)c2N(c2ccc(-c3ccc4ccccc4c3)cc2)c2ccc(-c3cccc4c3oc3ccccc34)cc2)cc1 ; c1ccc2cc(-c3ccc(N(c4ccc(-c5cccc6c5oc5ccccc56)cc4)c4c(-c5ccc6ccccc6c5)ccc5c4oc4ccccc45)cc3)ccc2c1]

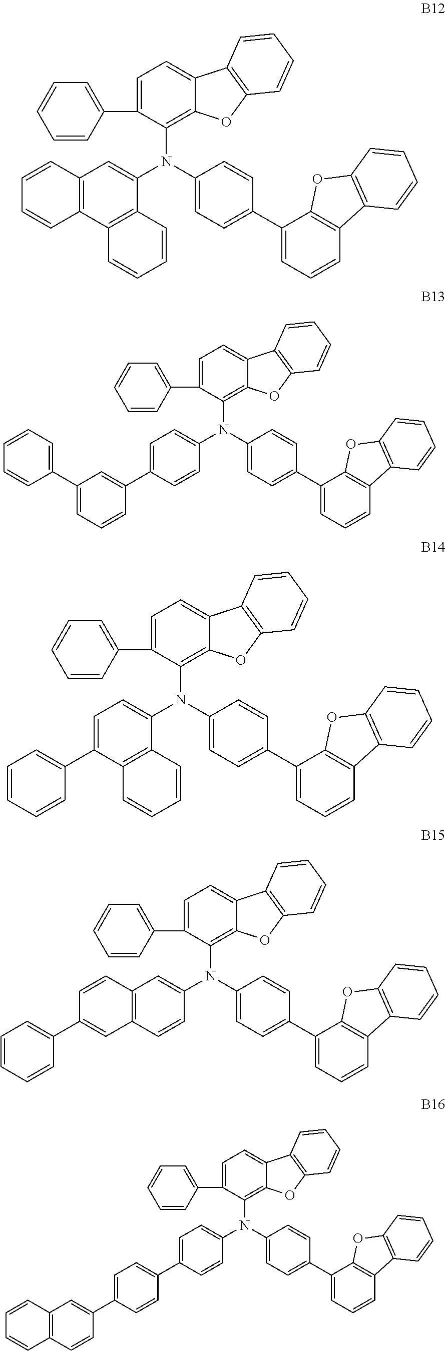 [SMILES: c1ccc(-c2ccc3c(oc4ccccc43)c2N(c2ccc(-c3cccc4c3oc3ccccc34)cc2)c2cc3ccccc3c3ccccc23)cc1 ; c1ccc(-c2cccc(-c3ccc(N(c4ccc(-c5cccc6c5oc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3cccc4c3oc3ccccc34)cc2)c2ccc(-c3ccccc3)c3ccccc23)cc1 ; c1ccc(-c2ccc3cc(N(c4ccc(-c5cccc6c5oc5ccccc56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4c3oc3ccccc34)cc2)cc1]

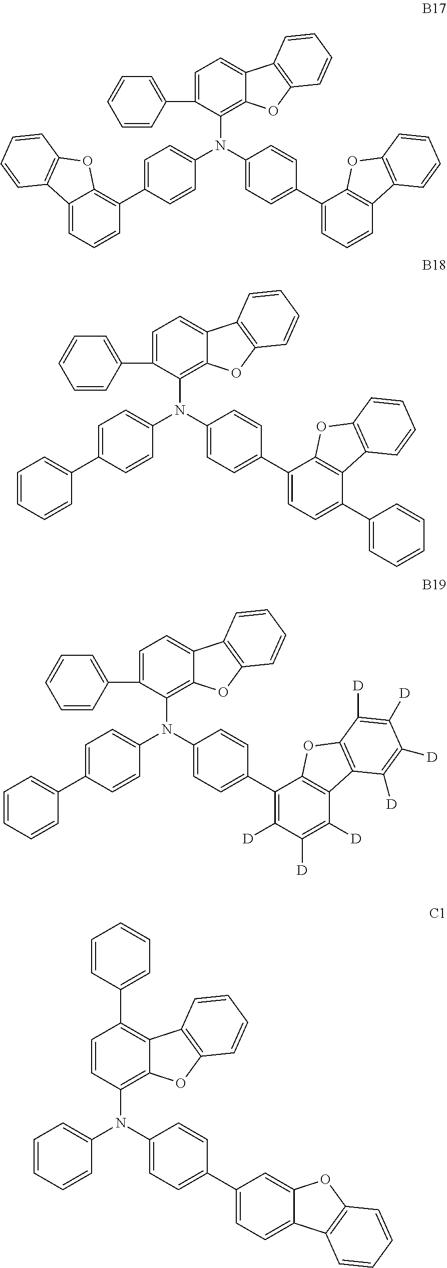 [SMILES: c1ccc(-c2ccc3c(oc4ccccc43)c2N(c2ccc(-c3cccc4c3oc3ccccc34)cc2)c2ccc(-c3cccc4c3oc3ccccc34)cc2)cc1 ; c1ccc(-c2ccc(N(c3ccc(-c4ccc(-c5ccccc5)c5c4oc4ccccc45)cc3)c3c(-c4ccccc4)ccc4c3oc3ccccc34)cc2)cc1 ; [2H]c1c([2H])c([2H])c2c(oc3c(-c4ccc(N(c5ccc(-c6ccccc6)cc5)c5c(-c6ccccc6)ccc6c5oc5ccccc56)cc4)c([2H])c([2H])c([2H])c32)c1[2H] ; c1ccc(-c2ccc(N(c3ccccc3)c3ccc(-c4ccc5c(c4)oc4ccccc45)cc3)c3oc4ccccc4c23)cc1]

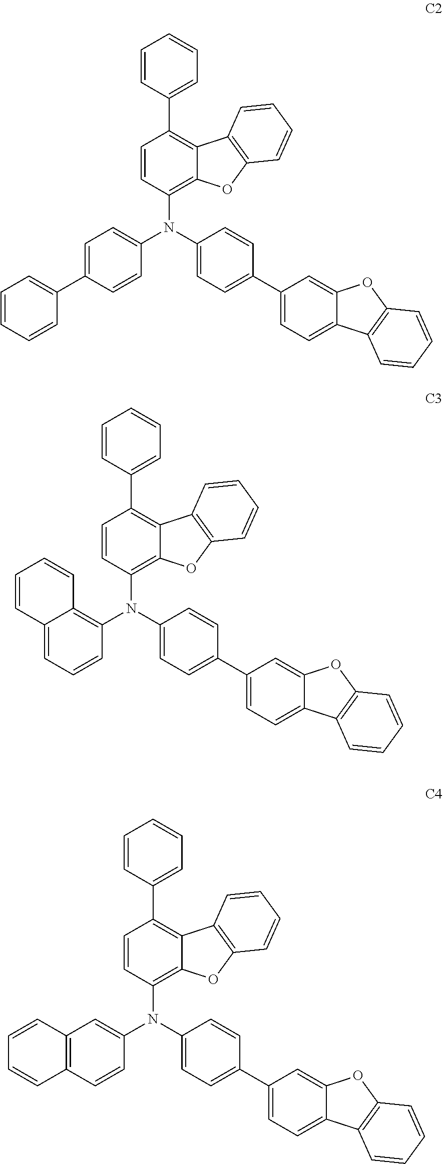 [SMILES: c1ccc(-c2ccc(N(c3ccc(-c4ccc5c(c4)oc4ccccc45)cc3)c3ccc(-c4ccccc4)c4c3oc3ccccc34)cc2)cc1 ; c1ccc(-c2ccc(N(c3ccc(-c4ccc5c(c4)oc4ccccc45)cc3)c3cccc4ccccc34)c3oc4ccccc4c23)cc1 ; c1ccc(-c2ccc(N(c3ccc(-c4ccc5c(c4)oc4ccccc45)cc3)c3ccc4ccccc4c3)c3oc4ccccc4c23)cc1]

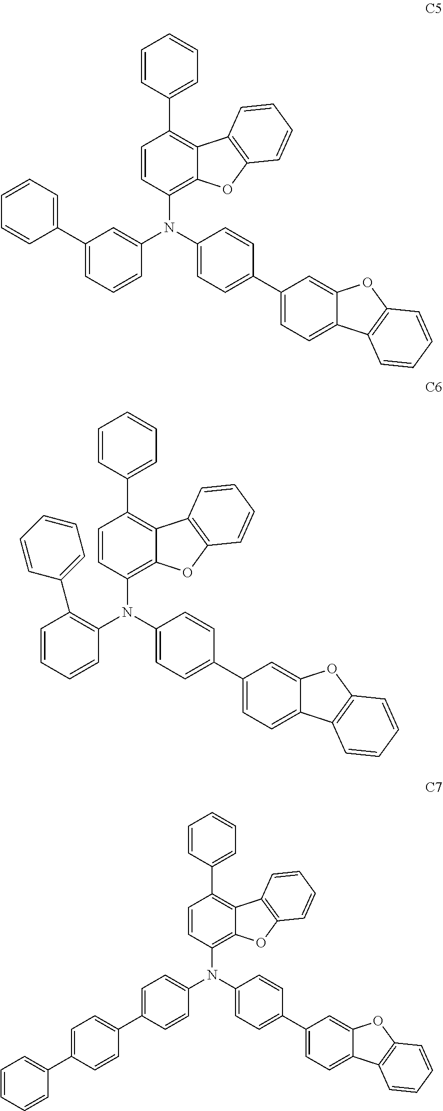 [SMILES: c1ccc(-c2cccc(N(c3ccc(-c4ccc5c(c4)oc4ccccc45)cc3)c3ccc(-c4ccccc4)c4c3oc3ccccc34)c2)cc1 ; c1ccc(-c2ccccc2N(c2ccc(-c3ccc4c(c3)oc3ccccc34)cc2)c2ccc(-c3ccccc3)c3c2oc2ccccc23)cc1 ; c1ccc(-c2ccc(-c3ccc(N(c4ccc(-c5ccc6c(c5)oc5ccccc56)cc4)c4ccc(-c5ccccc5)c5c4oc4ccccc45)cc3)cc2)cc1]

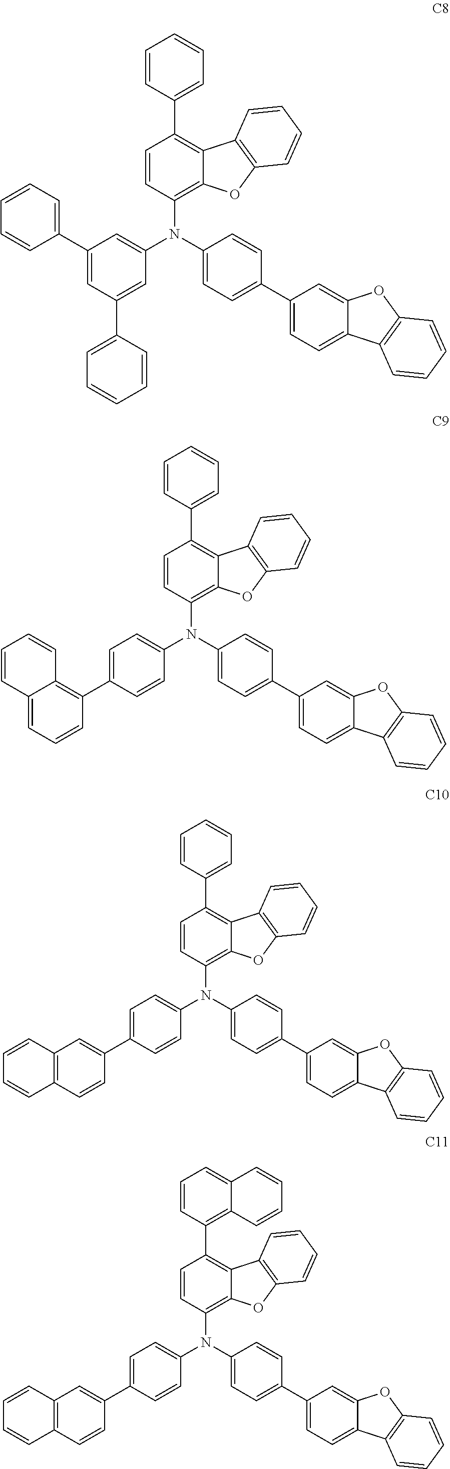 [SMILES: c1ccc(-c2cc(-c3ccccc3)cc(N(c3ccc(-c4ccc5c(c4)oc4ccccc45)cc3)c3ccc(-c4ccccc4)c4c3oc3ccccc34)c2)cc1 ; c1ccc(-c2ccc(N(c3ccc(-c4ccc5c(c4)oc4ccccc45)cc3)c3ccc(-c4cccc5ccccc45)cc3)c3oc4ccccc4c23)cc1 ; c1ccc(-c2ccc(N(c3ccc(-c4ccc5ccccc5c4)cc3)c3ccc(-c4ccc5c(c4)oc4ccccc45)cc3)c3oc4ccccc4c23)cc1 ; c1ccc2cc(-c3ccc(N(c4ccc(-c5ccc6c(c5)oc5ccccc56)cc4)c4ccc(-c5cccc6ccccc56)c5c4oc4ccccc45)cc3)ccc2c1]

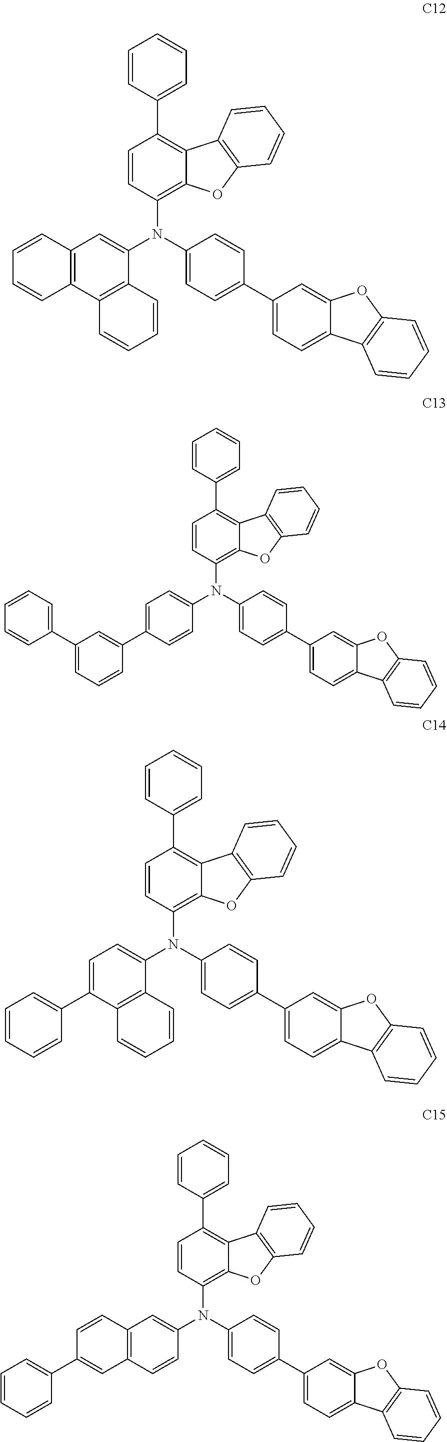 [SMILES: c1ccc(-c2ccc(N(c3ccc(-c4ccc5c(c4)oc4ccccc45)cc3)c3cc4ccccc4c4ccccc34)c3oc4ccccc4c23)cc1 ; c1ccc(-c2cccc(-c3ccc(N(c4ccc(-c5ccc6c(c5)oc5ccccc56)cc4)c4ccc(-c5ccccc5)c5c4oc4ccccc45)cc3)c2)cc1 ; c1ccc(-c2ccc(N(c3ccc(-c4ccc5c(c4)oc4ccccc45)cc3)c3ccc(-c4ccccc4)c4c3oc3ccccc34)c3ccccc23)cc1 ; c1ccc(-c2ccc3cc(N(c4ccc(-c5ccc6c(c5)oc5ccccc56)cc4)c4ccc(-c5ccccc5)c5c4oc4ccccc45)ccc3c2)cc1]

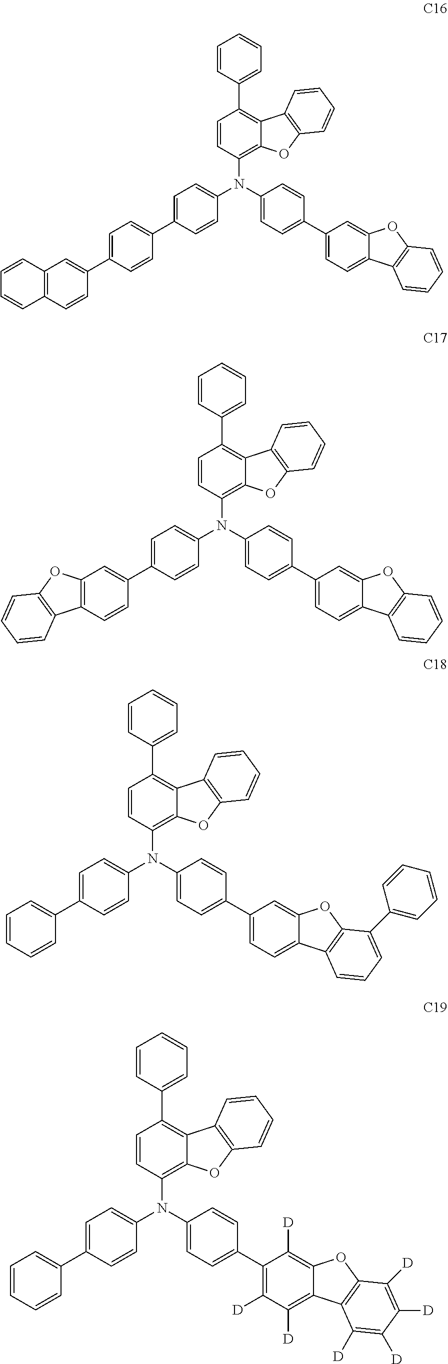 [SMILES: c1ccc(-c2ccc(N(c3ccc(-c4ccc(-c5ccc6ccccc6c5)cc4)cc3)c3ccc(-c4ccc5c(c4)oc4ccccc45)cc3)c3oc4ccccc4c23)cc1 ; c1ccc(-c2ccc(N(c3ccc(-c4ccc5c(c4)oc4ccccc45)cc3)c3ccc(-c4ccc5c(c4)oc4ccccc45)cc3)c3oc4ccccc4c23)cc1 ; c1ccc(-c2ccc(N(c3ccc(-c4ccc5c(c4)oc4c(-c6ccccc6)cccc45)cc3)c3ccc(-c4ccccc4)c4c3oc3ccccc34)cc2)cc1 ; [2H]c1c([2H])c([2H])c2c(oc3c([2H])c(-c4ccc(N(c5ccc(-c6ccccc6)cc5)c5ccc(-c6ccccc6)c6c5oc5ccccc56)cc4)c([2H])c([2H])c32)c1[2H]]

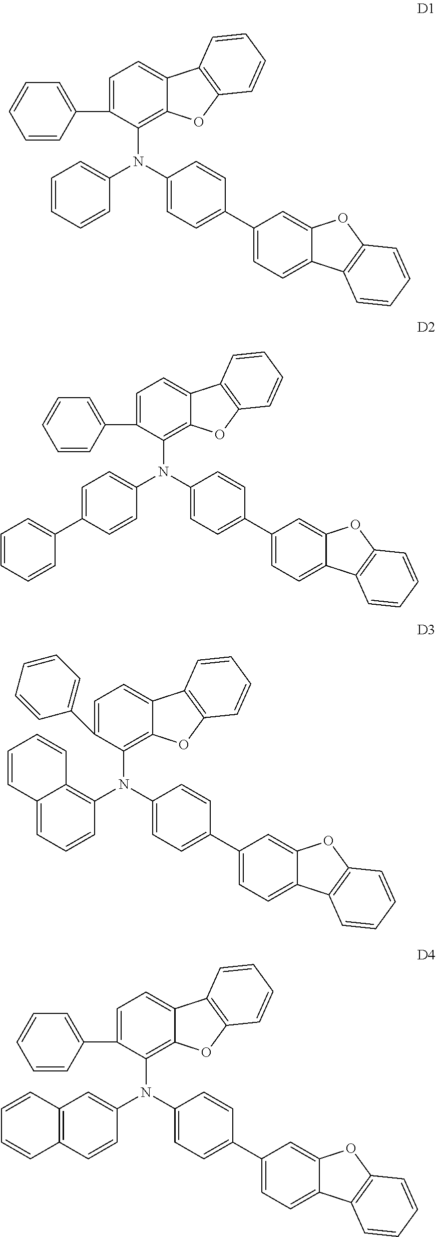 [SMILES: c1ccc(-c2ccc3c(oc4ccccc43)c2N(c2ccccc2)c2ccc(-c3ccc4c(c3)oc3ccccc34)cc2)cc1 ; c1ccc(-c2ccc(N(c3ccc(-c4ccc5c(c4)oc4ccccc45)cc3)c3c(-c4ccccc4)ccc4c3oc3ccccc34)cc2)cc1 ; c1ccc(-c2ccc3c(oc4ccccc43)c2N(c2ccc(-c3ccc4c(c3)oc3ccccc34)cc2)c2cccc3ccccc23)cc1 ; c1ccc(-c2ccc3c(oc4ccccc43)c2N(c2ccc(-c3ccc4c(c3)oc3ccccc34)cc2)c2ccc3ccccc3c2)cc1]

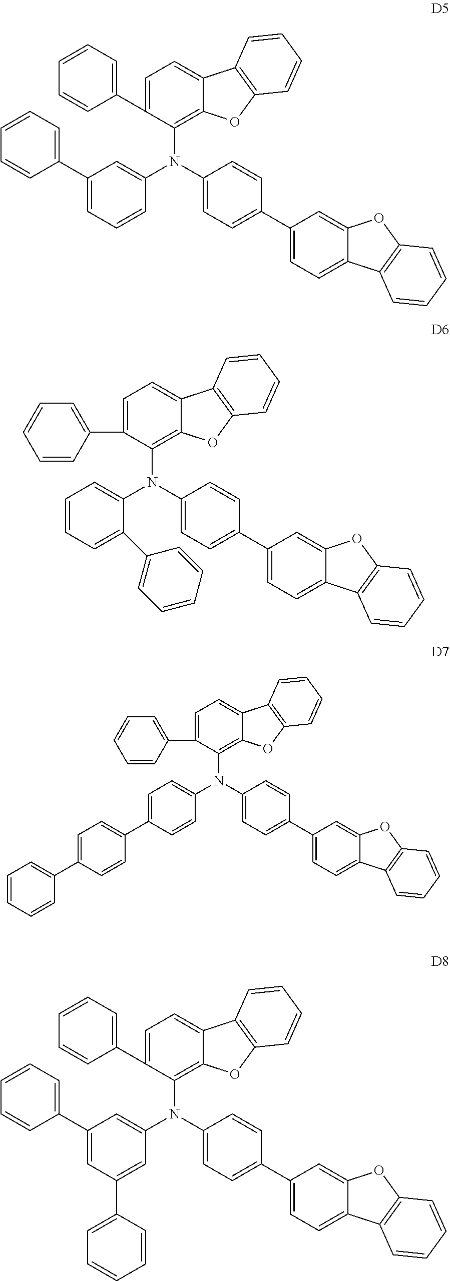 [SMILES: c1ccc(-c2cccc(N(c3ccc(-c4ccc5c(c4)oc4ccccc45)cc3)c3c(-c4ccccc4)ccc4c3oc3ccccc34)c2)cc1 ; c1ccc(-c2ccccc2N(c2ccc(-c3ccc4c(c3)oc3ccccc34)cc2)c2c(-c3ccccc3)ccc3c2oc2ccccc23)cc1 ; c1ccc(-c2ccc(-c3ccc(N(c4ccc(-c5ccc6c(c5)oc5ccccc56)cc4)c4c(-c5ccccc5)ccc5c4oc4ccccc45)cc3)cc2)cc1 ; c1ccc(-c2cc(-c3ccccc3)cc(N(c3ccc(-c4ccc5c(c4)oc4ccccc45)cc3)c3c(-c4ccccc4)ccc4c3oc3ccccc34)c2)cc1]

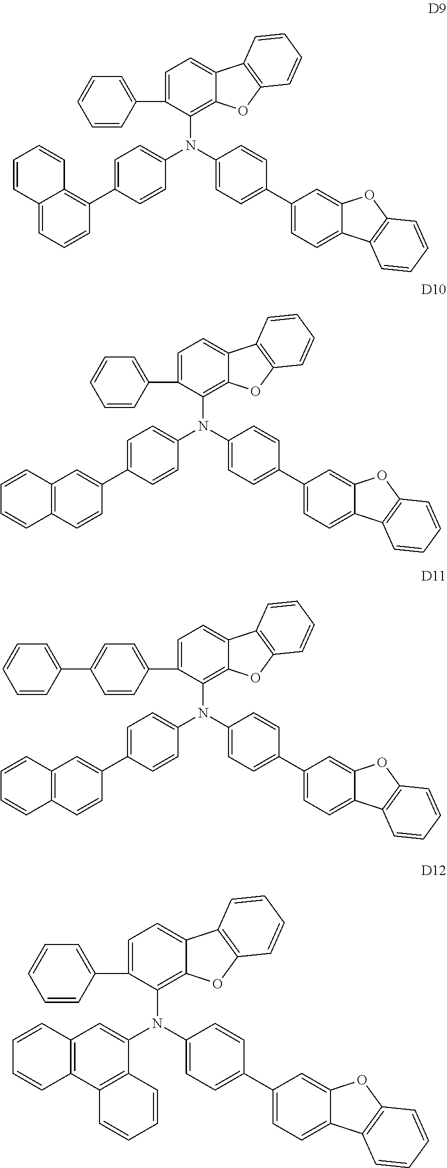 [SMILES: c1ccc(-c2ccc3c(oc4ccccc43)c2N(c2ccc(-c3ccc4c(c3)oc3ccccc34)cc2)c2ccc(-c3cccc4ccccc34)cc2)cc1 ; c1ccc(-c2ccc3c(oc4ccccc43)c2N(c2ccc(-c3ccc4ccccc4c3)cc2)c2ccc(-c3ccc4c(c3)oc3ccccc34)cc2)cc1 ; c1ccc(-c2ccc(-c3ccc4c(oc5ccccc54)c3N(c3ccc(-c4ccc5ccccc5c4)cc3)c3ccc(-c4ccc5c(c4)oc4ccccc45)cc3)cc2)cc1 ; c1ccc(-c2ccc3c(oc4ccccc43)c2N(c2ccc(-c3ccc4c(c3)oc3ccccc34)cc2)c2cc3ccccc3c3ccccc23)cc1]

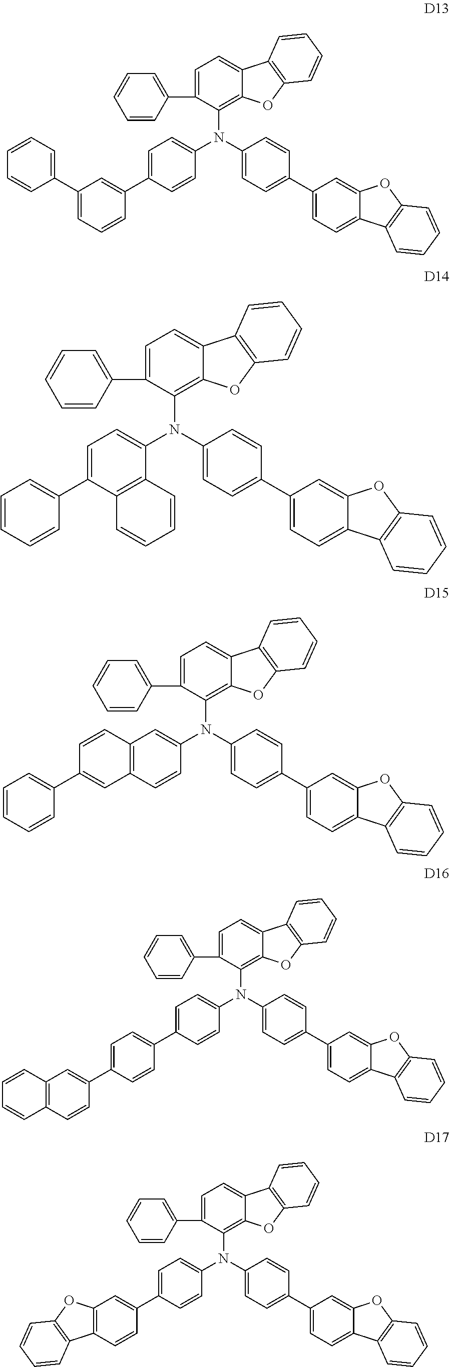 [SMILES: c1ccc(-c2cccc(-c3ccc(N(c4ccc(-c5ccc6c(c5)oc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3ccc4c(c3)oc3ccccc34)cc2)c2ccc(-c3ccccc3)c3ccccc23)cc1 ; c1ccc(-c2ccc3cc(N(c4ccc(-c5ccc6c(c5)oc5ccccc56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3ccc4c(c3)oc3ccccc34)cc2)cc1 ; c1ccc(-c2ccc3c(oc4ccccc43)c2N(c2ccc(-c3ccc4c(c3)oc3ccccc34)cc2)c2ccc(-c3ccc4c(c3)oc3ccccc34)cc2)cc1]

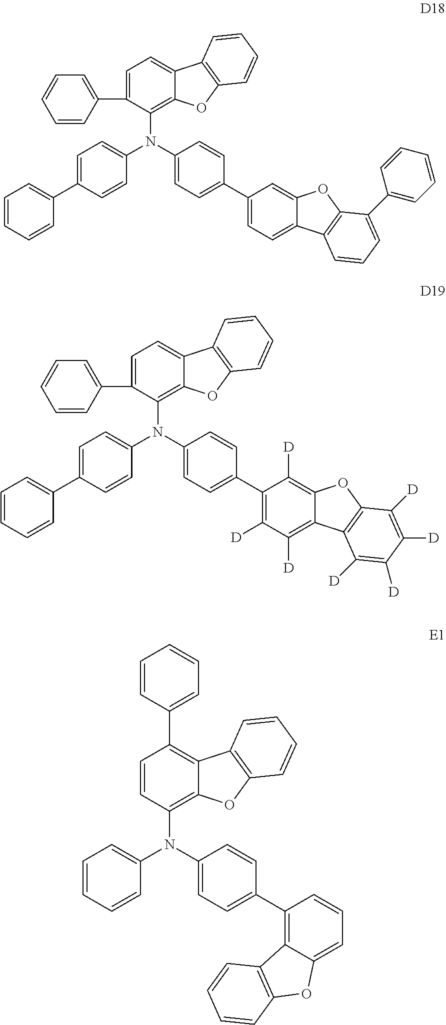 [SMILES: c1ccc(-c2ccc(N(c3ccc(-c4ccc5c(c4)oc4c(-c6ccccc6)cccc45)cc3)c3c(-c4ccccc4)ccc4c3oc3ccccc34)cc2)cc1 ; [2H]c1c([2H])c([2H])c2c(oc3c([2H])c(-c4ccc(N(c5ccc(-c6ccccc6)cc5)c5c(-c6ccccc6)ccc6c5oc5ccccc56)cc4)c([2H])c([2H])c32)c1[2H] ; c1ccc(-c2ccc(N(c3ccccc3)c3ccc(-c4cccc5oc6ccccc6c45)cc3)c3oc4ccccc4c23)cc1]

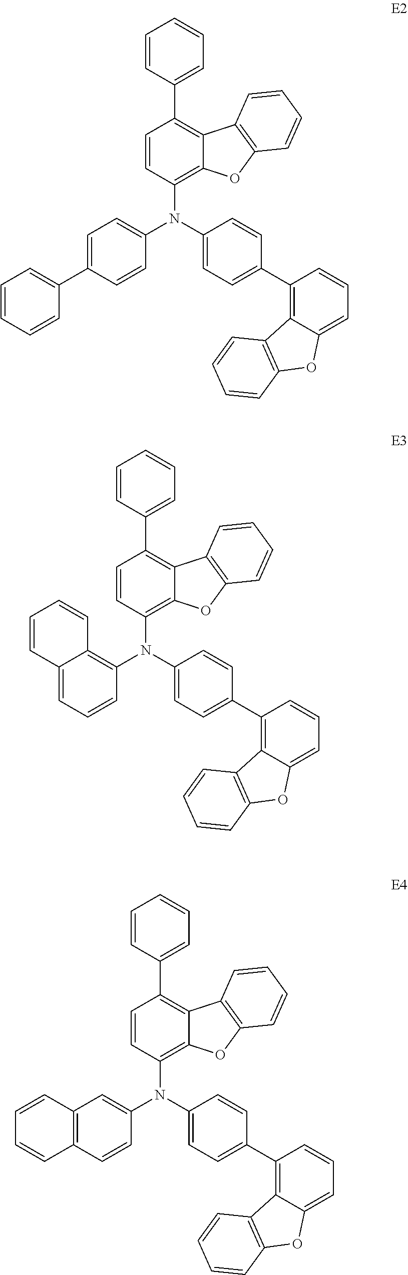 [SMILES: c1ccc(-c2ccc(N(c3ccc(-c4cccc5oc6ccccc6c45)cc3)c3ccc(-c4ccccc4)c4c3oc3ccccc34)cc2)cc1 ; c1ccc(-c2ccc(N(c3ccc(-c4cccc5oc6ccccc6c45)cc3)c3cccc4ccccc34)c3oc4ccccc4c23)cc1 ; c1ccc(-c2ccc(N(c3ccc(-c4cccc5oc6ccccc6c45)cc3)c3ccc4ccccc4c3)c3oc4ccccc4c23)cc1]

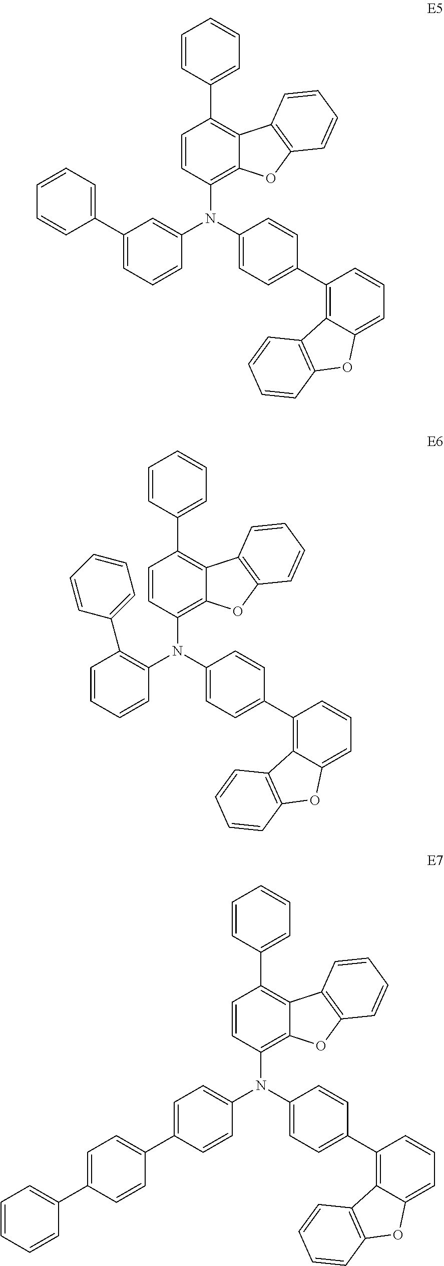 [SMILES: c1ccc(-c2cccc(N(c3ccc(-c4cccc5oc6ccccc6c45)cc3)c3ccc(-c4ccccc4)c4c3oc3ccccc34)c2)cc1 ; c1ccc(-c2ccccc2N(c2ccc(-c3cccc4oc5ccccc5c34)cc2)c2ccc(-c3ccccc3)c3c2oc2ccccc23)cc1 ; c1ccc(-c2ccc(-c3ccc(N(c4ccc(-c5cccc6oc7ccccc7c56)cc4)c4ccc(-c5ccccc5)c5c4oc4ccccc45)cc3)cc2)cc1]

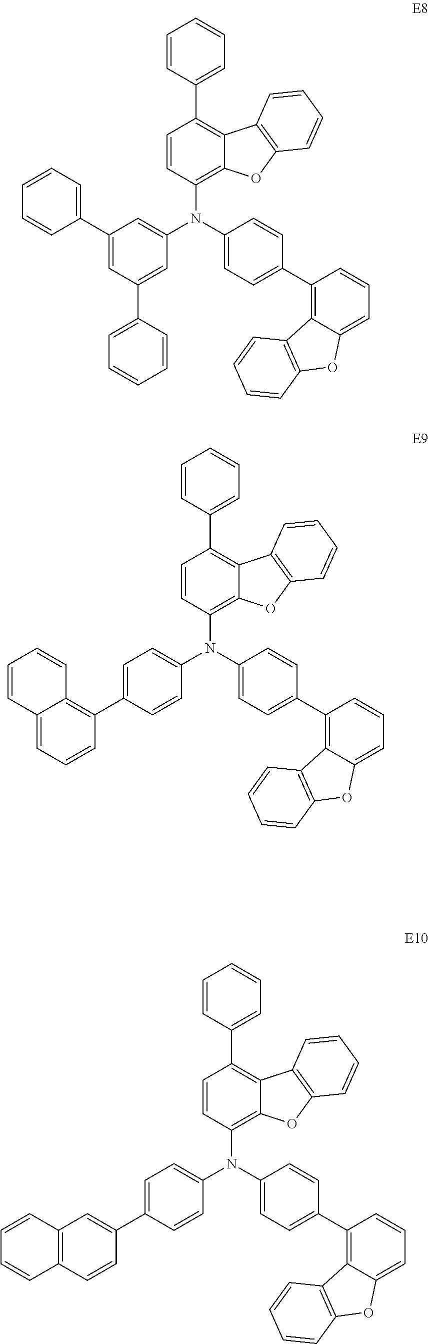 [SMILES: c1ccc(-c2cc(-c3ccccc3)cc(N(c3ccc(-c4cccc5oc6ccccc6c45)cc3)c3ccc(-c4ccccc4)c4c3oc3ccccc34)c2)cc1 ; c1ccc(-c2ccc(N(c3ccc(-c4cccc5ccccc45)cc3)c3ccc(-c4cccc5oc6ccccc6c45)cc3)c3oc4ccccc4c23)cc1 ; c1ccc(-c2ccc(N(c3ccc(-c4ccc5ccccc5c4)cc3)c3ccc(-c4cccc5oc6ccccc6c45)cc3)c3oc4ccccc4c23)cc1]

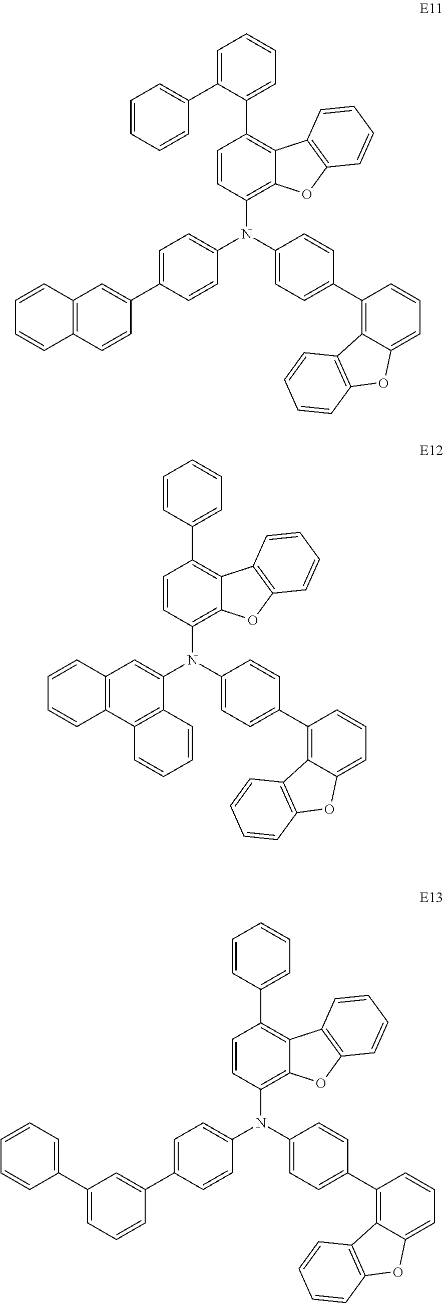 [SMILES: c1ccc(-c2ccccc2-c2ccc(N(c3ccc(-c4ccc5ccccc5c4)cc3)c3ccc(-c4cccc5oc6ccccc6c45)cc3)c3oc4ccccc4c23)cc1 ; c1ccc(-c2ccc(N(c3ccc(-c4cccc5oc6ccccc6c45)cc3)c3cc4ccccc4c4ccccc34)c3oc4ccccc4c23)cc1 ; c1ccc(-c2cccc(-c3ccc(N(c4ccc(-c5cccc6oc7ccccc7c56)cc4)c4ccc(-c5ccccc5)c5c4oc4ccccc45)cc3)c2)cc1]

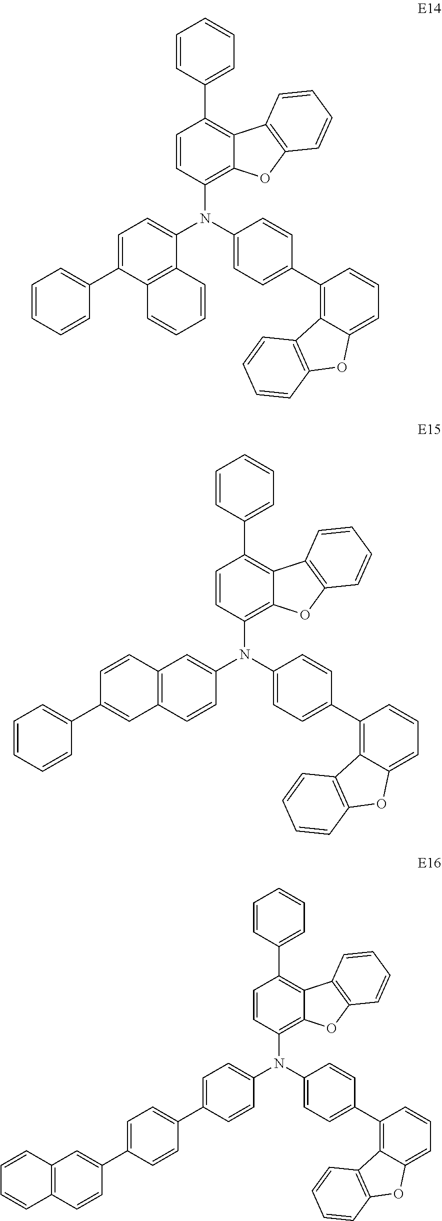 [SMILES: c1ccc(-c2ccc(N(c3ccc(-c4cccc5oc6ccccc6c45)cc3)c3ccc(-c4ccccc4)c4c3oc3ccccc34)c3ccccc23)cc1 ; c1ccc(-c2ccc3cc(N(c4ccc(-c5cccc6oc7ccccc7c56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4cccc5oc6ccccc6c45)cc3)c3oc4ccccc4c23)cc1]

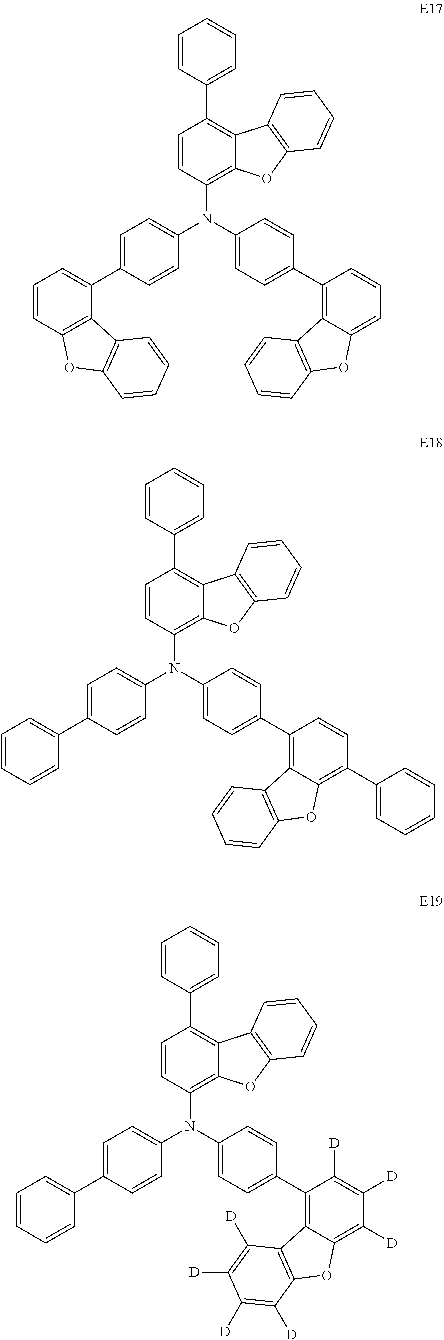 [SMILES: c1ccc(-c2ccc(N(c3ccc(-c4cccc5oc6ccccc6c45)cc3)c3ccc(-c4cccc5oc6ccccc6c45)cc3)c3oc4ccccc4c23)cc1 ; c1ccc(-c2ccc(N(c3ccc(-c4ccc(-c5ccccc5)c5oc6ccccc6c45)cc3)c3ccc(-c4ccccc4)c4c3oc3ccccc34)cc2)cc1 ; [2H]c1c([2H])c([2H])c2c(oc3c([2H])c([2H])c([2H])c(-c4ccc(N(c5ccc(-c6ccccc6)cc5)c5ccc(-c6ccccc6)c6c5oc5ccccc56)cc4)c32)c1[2H]]

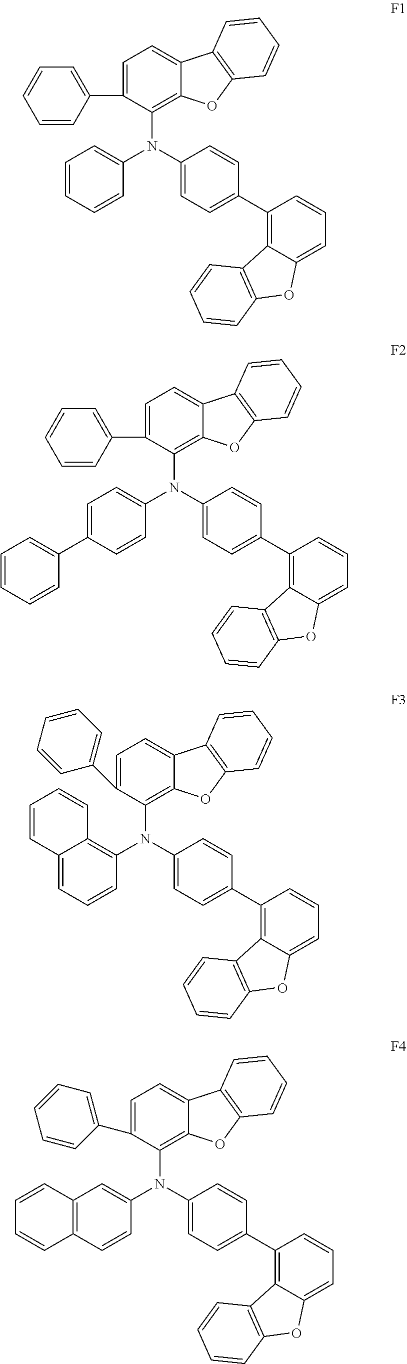

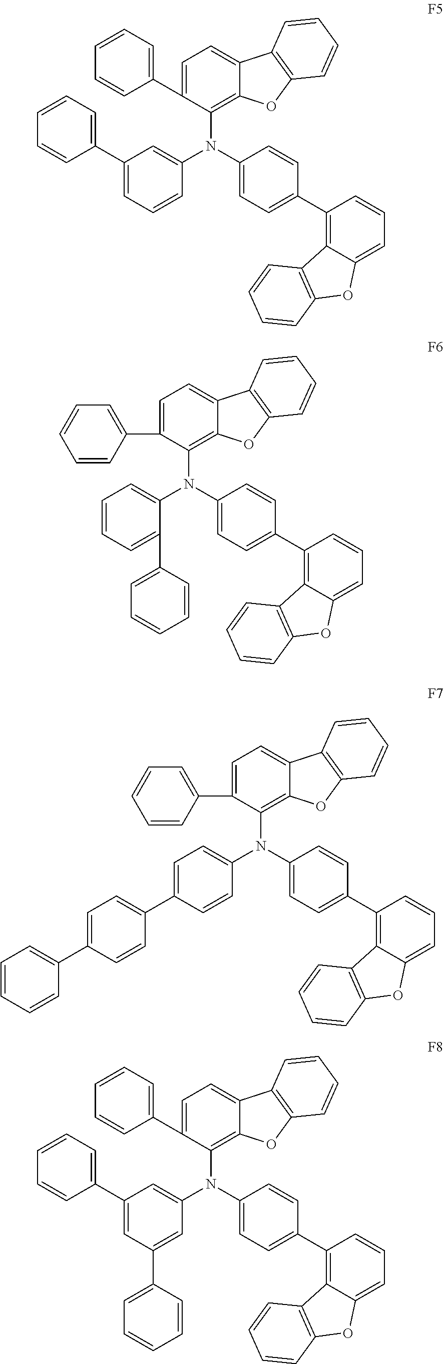

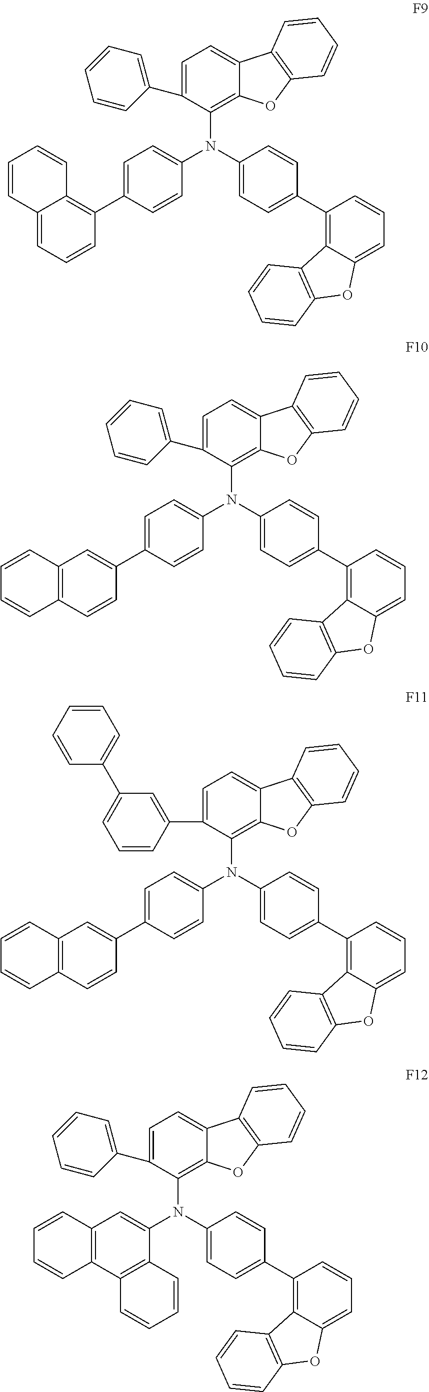

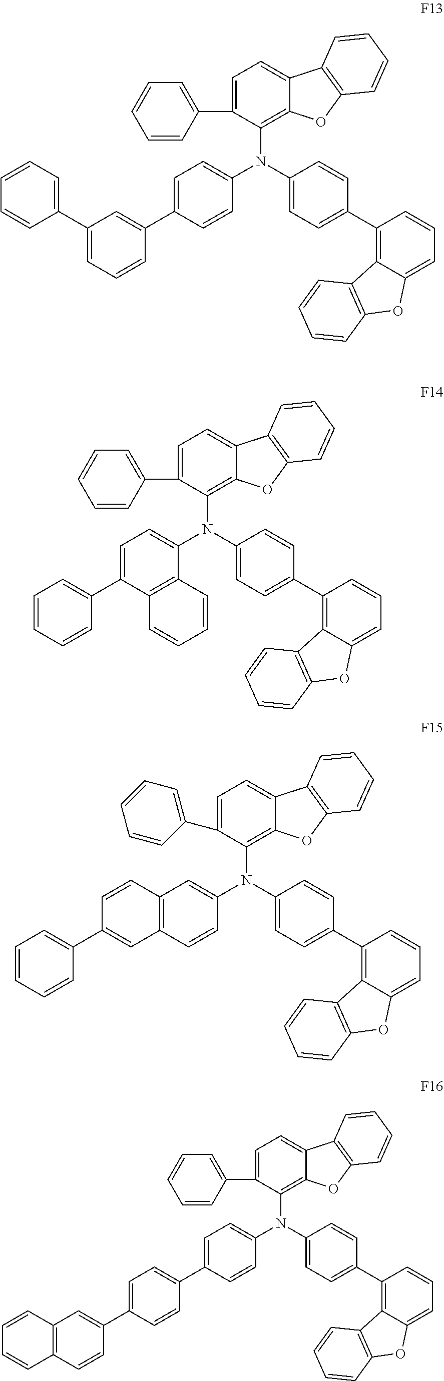 [SMILES: c1ccc(-c2cccc(-c3ccc(N(c4ccc(-c5cccc6oc7ccccc7c56)cc4)c4c(-c5ccccc5)ccc5c4oc4ccccc45)cc3)c2)cc1 ; c1ccc(-c2ccc3c(oc4ccccc43)c2N(c2ccc(-c3cccc4oc5ccccc5c34)cc2)c2ccc(-c3ccccc3)c3ccccc23)cc1 ; c1ccc(-c2ccc3cc(N(c4ccc(-c5cccc6oc7ccccc7c56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4oc5ccccc5c34)cc2)cc1]

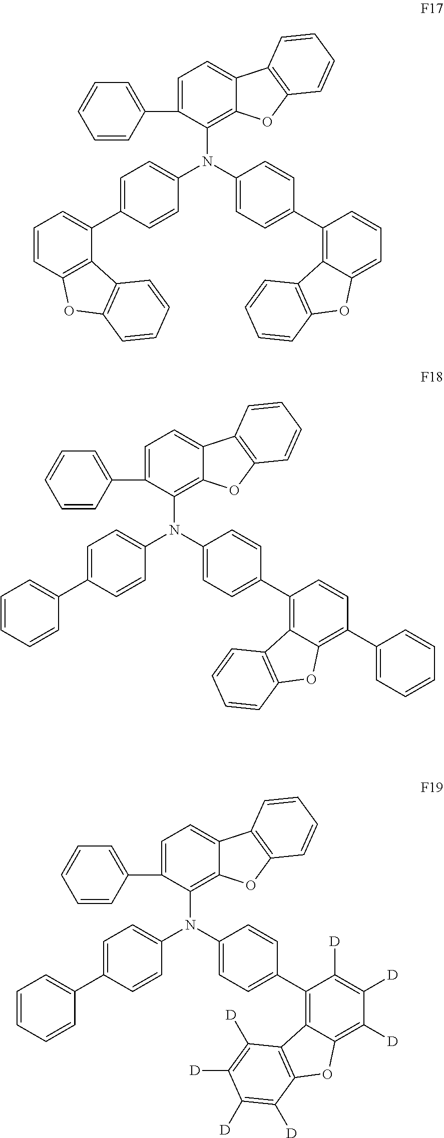 [SMILES: c1ccc(-c2ccc3c(oc4ccccc43)c2N(c2ccc(-c3cccc4oc5ccccc5c34)cc2)c2ccc(-c3cccc4oc5ccccc5c34)cc2)cc1 ; c1ccc(-c2ccc(N(c3ccc(-c4ccc(-c5ccccc5)c5oc6ccccc6c45)cc3)c3c(-c4ccccc4)ccc4c3oc3ccccc34)cc2)cc1 ; [2H]c1c([2H])c([2H])c2c(oc3c([2H])c([2H])c([2H])c(-c4ccc(N(c5ccc(-c6ccccc6)cc5)c5c(-c6ccccc6)ccc6c5oc5ccccc56)cc4)c32)c1[2H]]

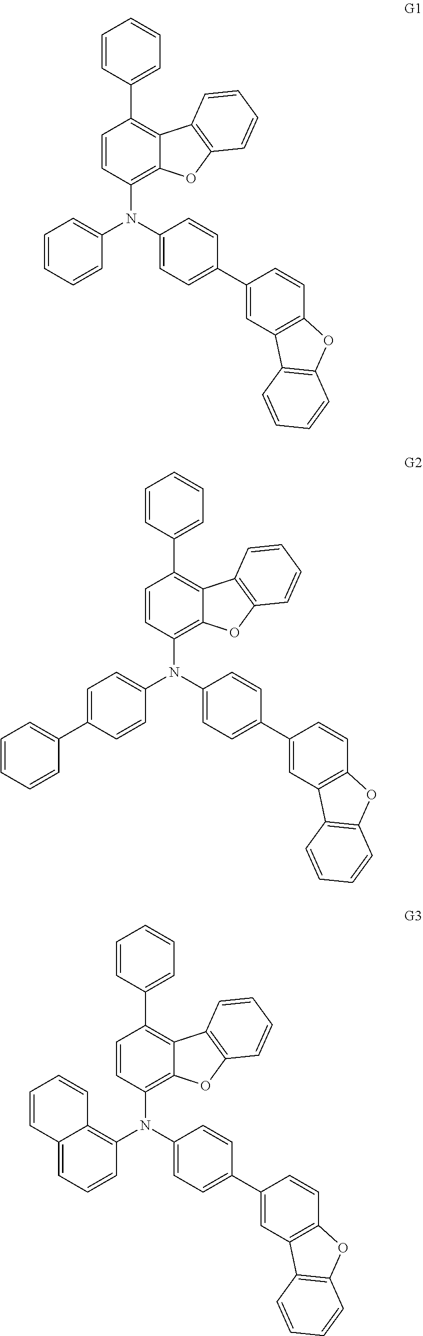 [SMILES: c1ccc(-c2ccc(N(c3ccccc3)c3ccc(-c4ccc5oc6ccccc6c5c4)cc3)c3oc4ccccc4c23)cc1 ; c1ccc(-c2ccc(N(c3ccc(-c4ccc5oc6ccccc6c5c4)cc3)c3ccc(-c4ccccc4)c4c3oc3ccccc34)cc2)cc1 ; c1ccc(-c2ccc(N(c3ccc(-c4ccc5oc6ccccc6c5c4)cc3)c3cccc4ccccc34)c3oc4ccccc4c23)cc1]

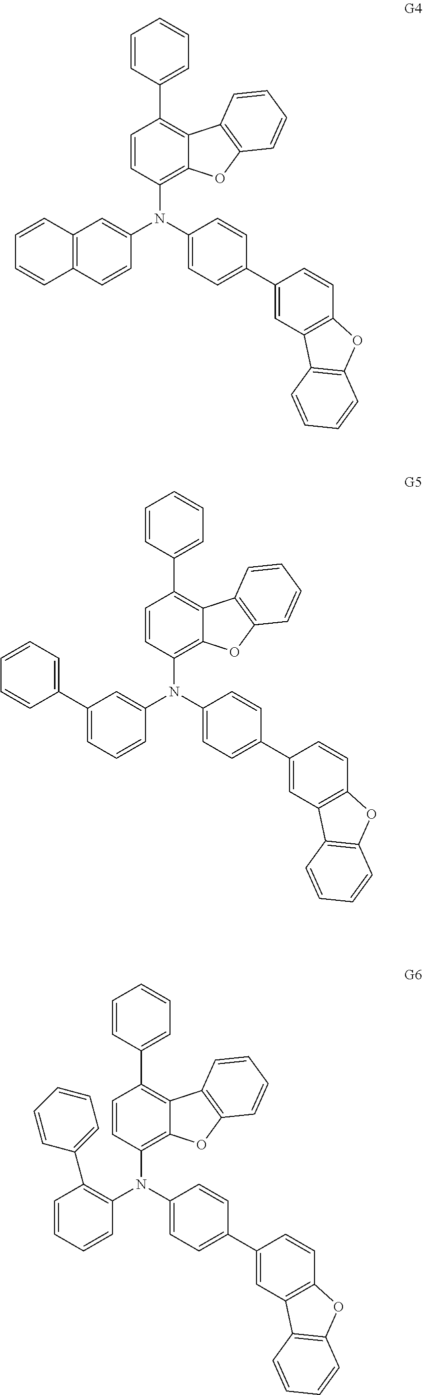 [SMILES: c1ccc(-c2ccc(N(c3ccc(-c4ccc5oc6ccccc6c5c4)cc3)c3ccc4ccccc4c3)c3oc4ccccc4c23)cc1 ; c1ccc(-c2cccc(N(c3ccc(-c4ccc5oc6ccccc6c5c4)cc3)c3ccc(-c4ccccc4)c4c3oc3ccccc34)c2)cc1 ; c1ccc(-c2ccccc2N(c2ccc(-c3ccc4oc5ccccc5c4c3)cc2)c2ccc(-c3ccccc3)c3c2oc2ccccc23)cc1]

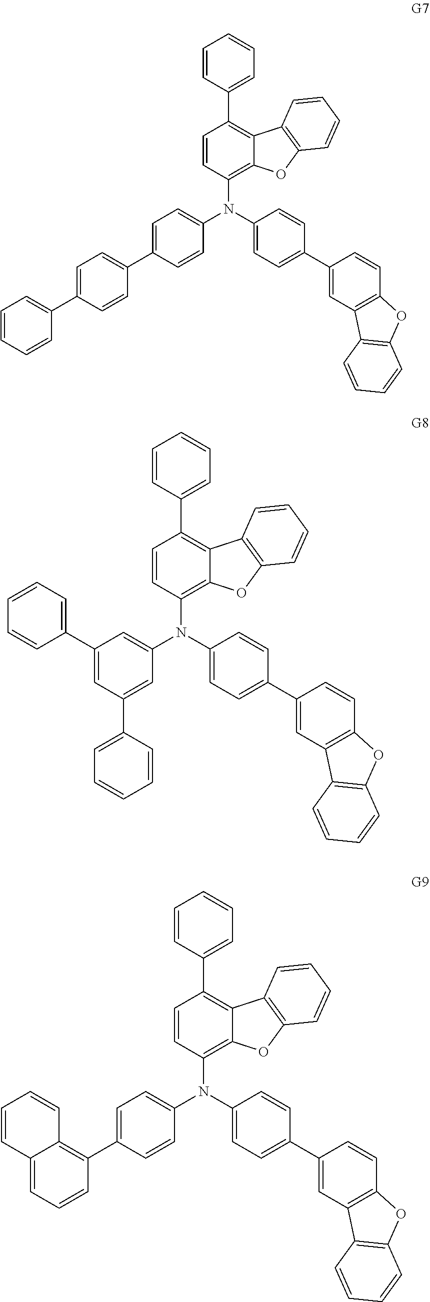 [SMILES: c1ccc(-c2ccc(-c3ccc(N(c4ccc(-c5ccc6oc7ccccc7c6c5)cc4)c4ccc(-c5ccccc5)c5c4oc4ccccc45)cc3)cc2)cc1 ; c1ccc(-c2cc(-c3ccccc3)cc(N(c3ccc(-c4ccc5oc6ccccc6c5c4)cc3)c3ccc(-c4ccccc4)c4c3oc3ccccc34)c2)cc1 ; c1ccc(-c2ccc(N(c3ccc(-c4ccc5oc6ccccc6c5c4)cc3)c3ccc(-c4cccc5ccccc45)cc3)c3oc4ccccc4c23)cc1]

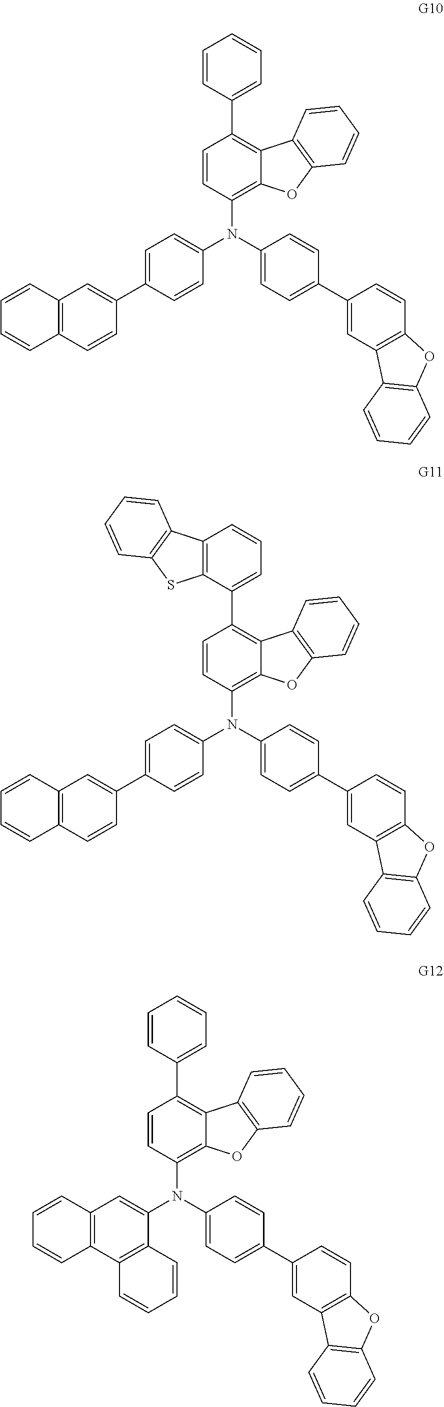 [SMILES: c1ccc(-c2ccc(N(c3ccc(-c4ccc5ccccc5c4)cc3)c3ccc(-c4ccc5oc6ccccc6c5c4)cc3)c3oc4ccccc4c23)cc1 ; c1ccc2cc(-c3ccc(N(c4ccc(-c5ccc6oc7ccccc7c6c5)cc4)c4ccc(-c5cccc6c5sc5ccccc56)c5c4oc4ccccc45)cc3)ccc2c1 ; c1ccc(-c2ccc(N(c3ccc(-c4ccc5oc6ccccc6c5c4)cc3)c3cc4ccccc4c4ccccc34)c3oc4ccccc4c23)cc1]

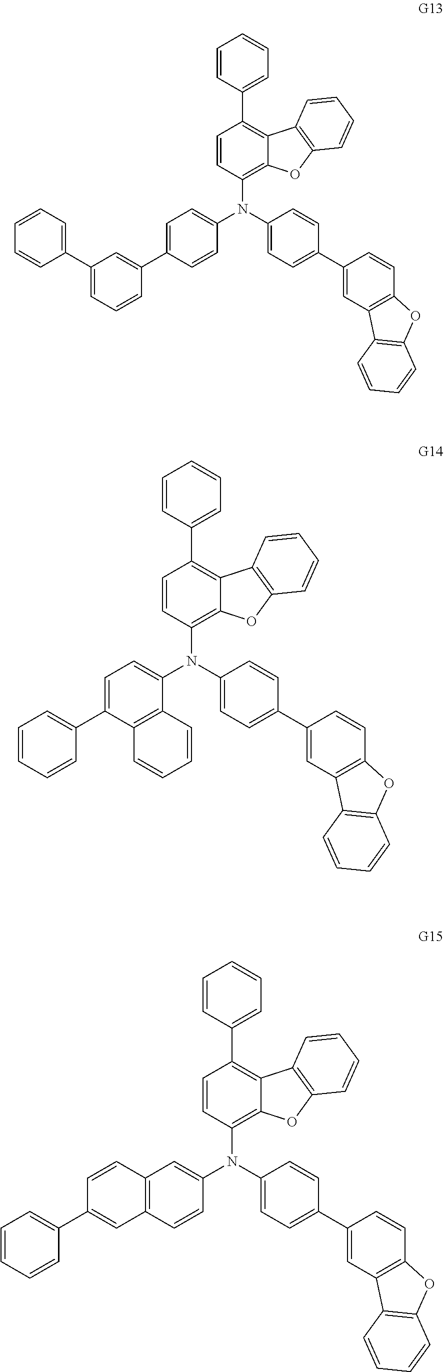 [SMILES: c1ccc(-c2cccc(-c3ccc(N(c4ccc(-c5ccc6oc7ccccc7c6c5)cc4)c4ccc(-c5ccccc5)c5c4oc4ccccc45)cc3)c2)cc1 ; c1ccc(-c2ccc(N(c3ccc(-c4ccc5oc6ccccc6c5c4)cc3)c3ccc(-c4ccccc4)c4c3oc3ccccc34)c3ccccc23)cc1 ; c1ccc(-c2ccc3cc(N(c4ccc(-c5ccc6oc7ccccc7c6c5)cc4)c4ccc(-c5ccccc5)c5c4oc4ccccc45)ccc3c2)cc1]

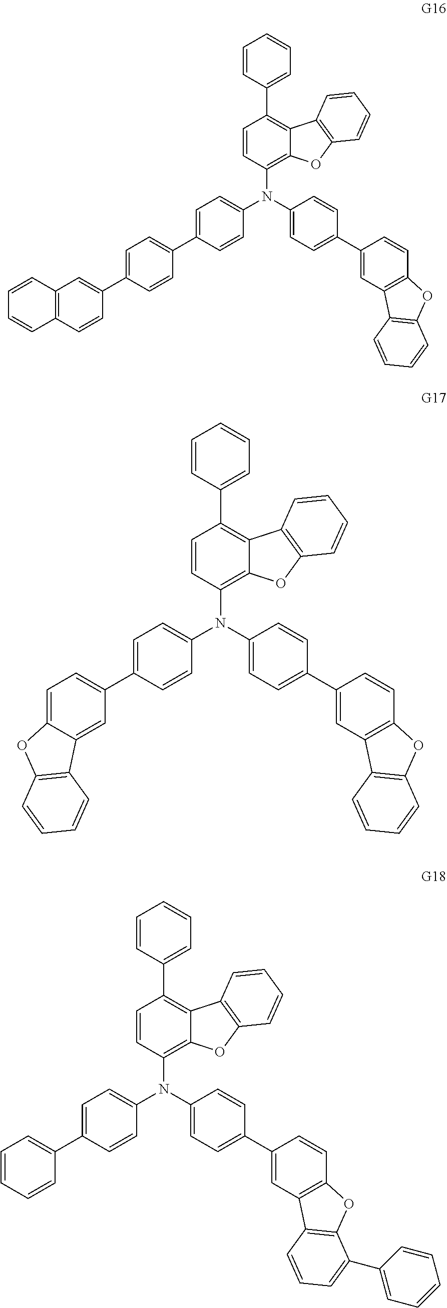 [SMILES: c1ccc(-c2ccc(N(c3ccc(-c4ccc(-c5ccc6ccccc6c5)cc4)cc3)c3ccc(-c4ccc5oc6ccccc6c5c4)cc3)c3oc4ccccc4c23)cc1 ; c1ccc(-c2ccc(N(c3ccc(-c4ccc5oc6ccccc6c5c4)cc3)c3ccc(-c4ccc5oc6ccccc6c5c4)cc3)c3oc4ccccc4c23)cc1 ; c1ccc(-c2ccc(N(c3ccc(-c4ccc5oc6c(-c7ccccc7)cccc6c5c4)cc3)c3ccc(-c4ccccc4)c4c3oc3ccccc34)cc2)cc1]

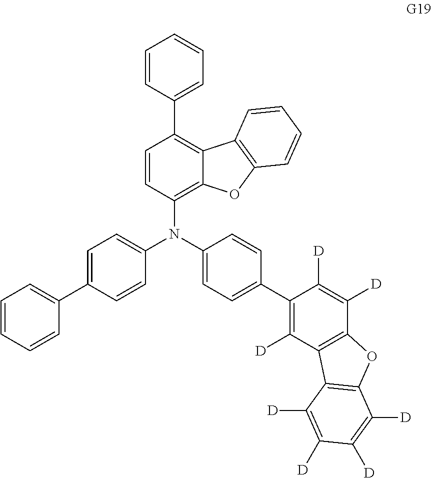 [SMILES: [2H]c1c([2H])c([2H])c2c(oc3c([2H])c([2H])c(-c4ccc(N(c5ccc(-c6ccccc6)cc5)c5ccc(-c6ccccc6)c6c5oc5ccccc56)cc4)c([2H])c32)c1[2H]]

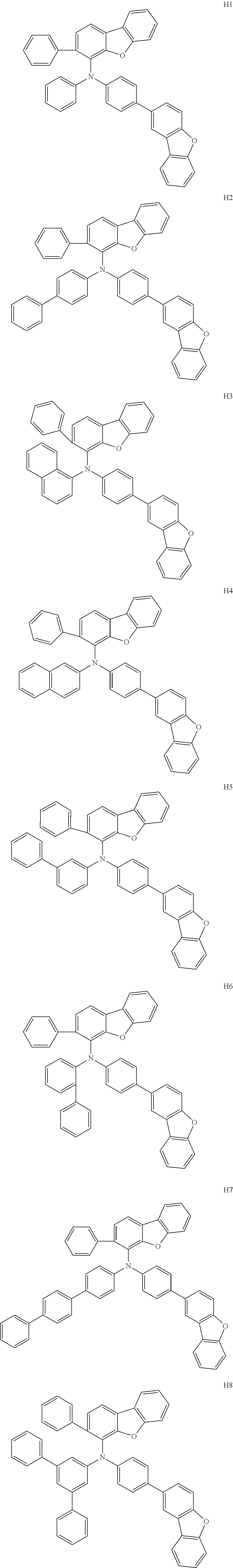

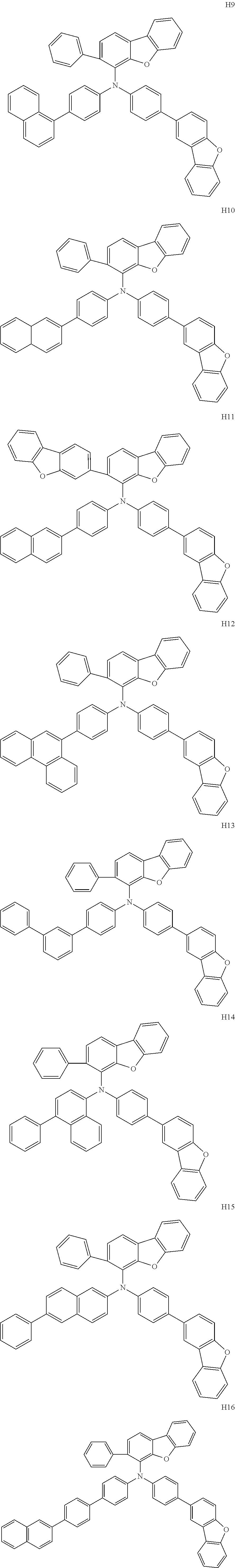

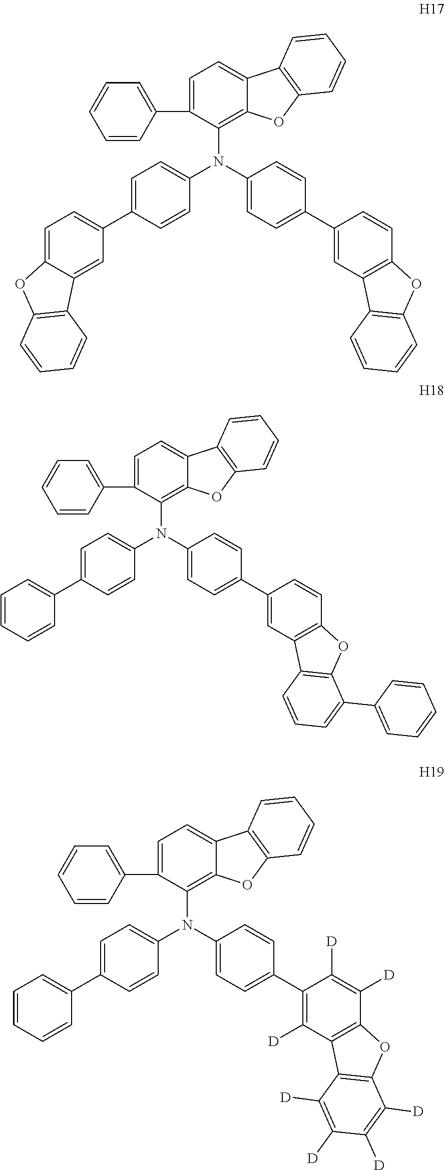 [SMILES: c1ccc(-c2ccc3c(oc4ccccc43)c2N(c2ccc(-c3ccc4oc5ccccc5c4c3)cc2)c2ccc(-c3ccc4oc5ccccc5c4c3)cc2)cc1 ; c1ccc(-c2ccc(N(c3ccc(-c4ccc5oc6c(-c7ccccc7)cccc6c5c4)cc3)c3c(-c4ccccc4)ccc4c3oc3ccccc34)cc2)cc1 ; [2H]c1c([2H])c([2H])c2c(oc3c([2H])c([2H])c(-c4ccc(N(c5ccc(-c6ccccc6)cc5)c5c(-c6ccccc6)ccc6c5oc5ccccc56)cc4)c([2H])c32)c1[2H]]

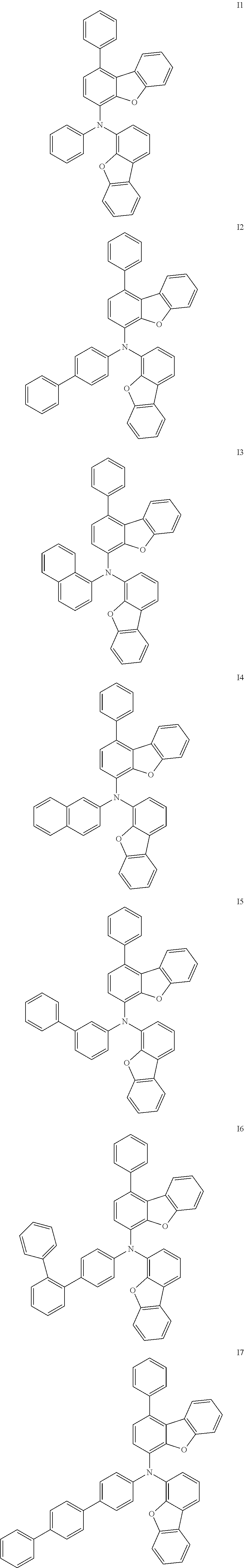

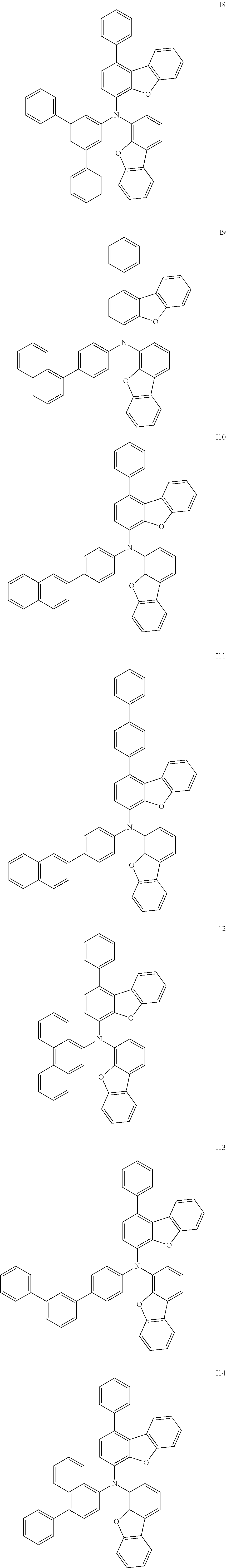

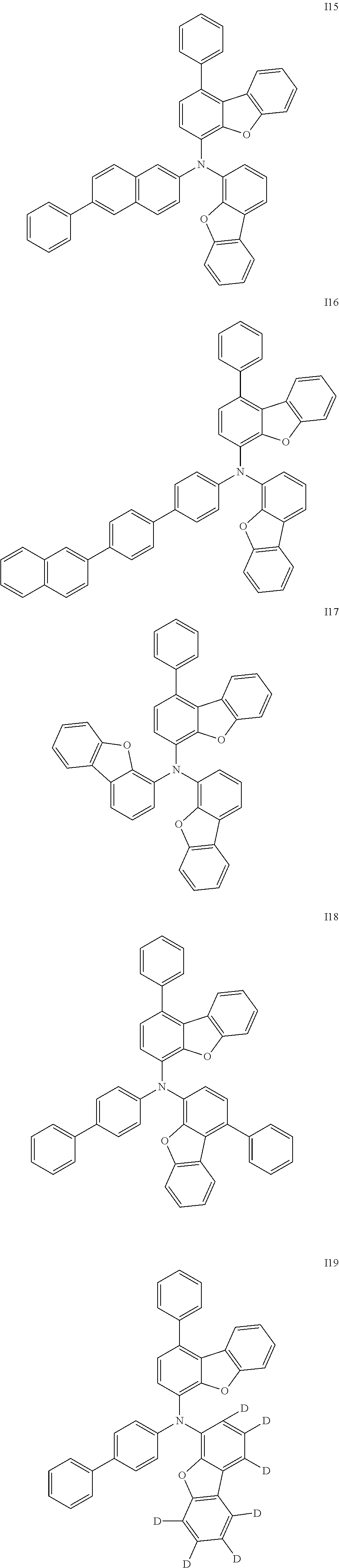 [SMILES: c1ccc(-c2ccc3cc(N(c4cccc5c4oc4ccccc45)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3cccc4c3oc3ccccc34)c3oc4ccccc4c23)cc1 ; c1ccc(-c2ccc(N(c3cccc4c3oc3ccccc34)c3cccc4c3oc3ccccc34)c3oc4ccccc4c23)cc1 ; c1ccc(-c2ccc(N(c3ccc(-c4ccccc4)c4c3oc3ccccc34)c3ccc(-c4ccccc4)c4c3oc3ccccc34)cc2)cc1 ; [2H]c1c([2H])c([2H])c2c(oc3c(N(c4ccc(-c5ccccc5)cc4)c4ccc(-c5ccccc5)c5c4oc4ccccc45)c([2H])c([2H])c([2H])c32)c1[2H]]

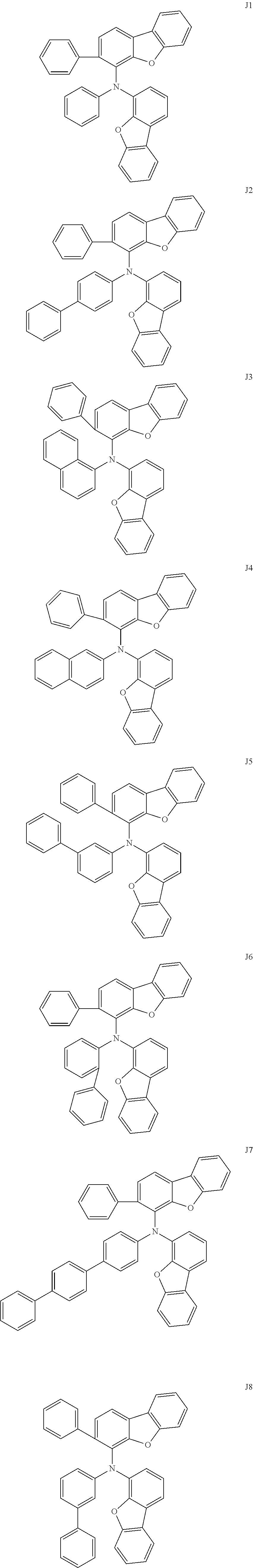

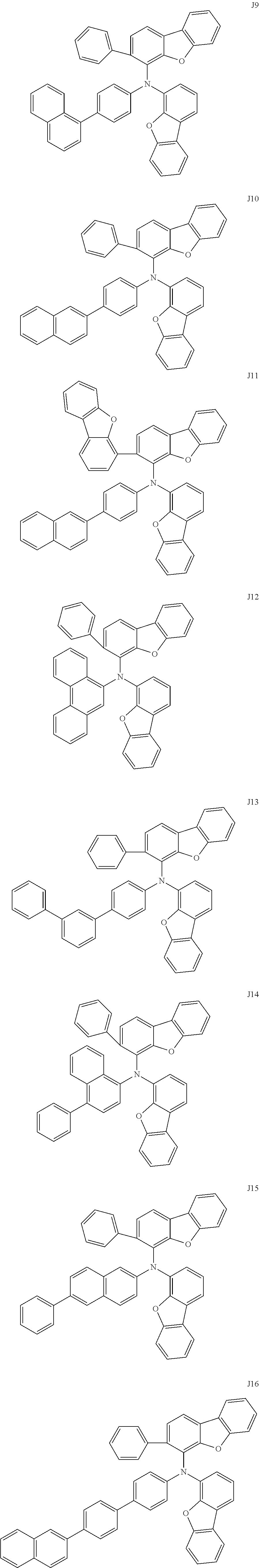

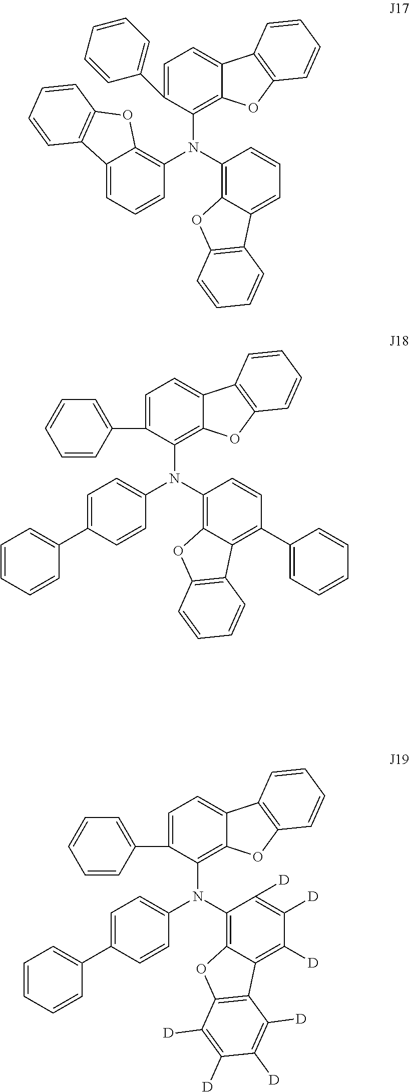 [SMILES: c1ccc(-c2ccc3c(oc4ccccc43)c2N(c2cccc3c2oc2ccccc23)c2cccc3c2oc2ccccc23)cc1 ; c1ccc(-c2ccc(N(c3c(-c4ccccc4)ccc4c3oc3ccccc34)c3ccc(-c4ccccc4)c4c3oc3ccccc34)cc2)cc1 ; [2H]c1c([2H])c([2H])c2c(oc3c(N(c4ccc(-c5ccccc5)cc4)c4c(-c5ccccc5)ccc5c4oc4ccccc45)c([2H])c([2H])c([2H])c32)c1[2H]]

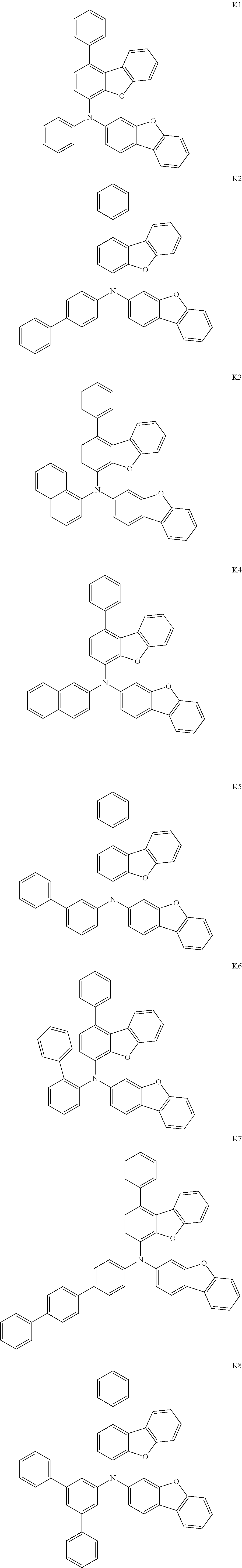

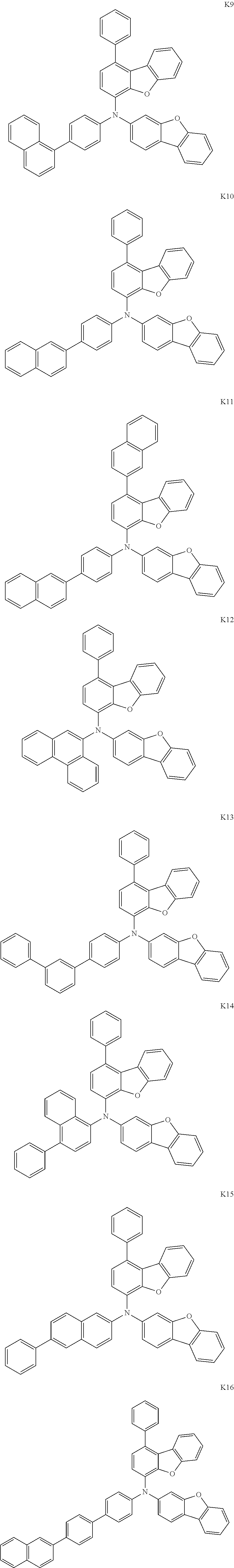

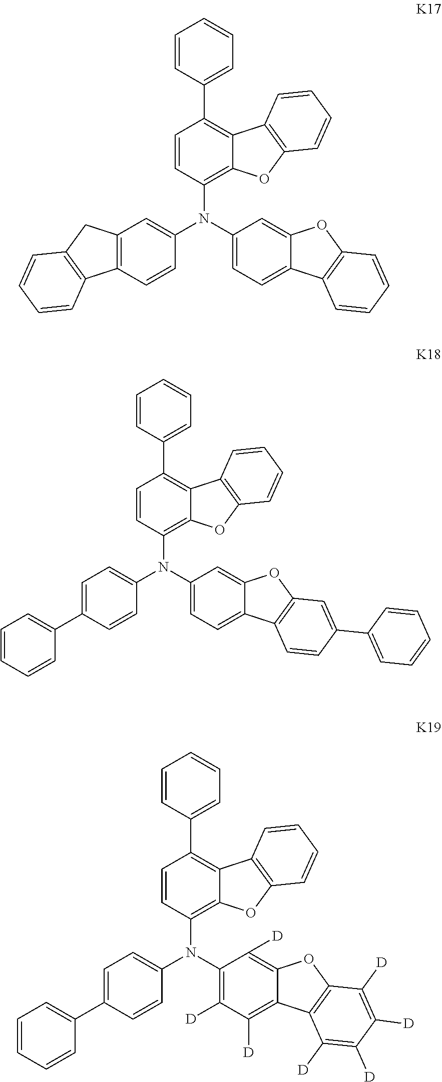 [SMILES: c1ccc(-c2ccc(N(c3ccc4c(c3)Cc3ccccc3-4)c3ccc4c(c3)oc3ccccc34)c3oc4ccccc4c23)cc1 ; c1ccc(-c2ccc(N(c3ccc4c(c3)oc3cc(-c5ccccc5)ccc34)c3ccc(-c4ccccc4)c4c3oc3ccccc34)cc2)cc1 ; [2H]c1c([2H])c([2H])c2c(oc3c([2H])c(N(c4ccc(-c5ccccc5)cc4)c4ccc(-c5ccccc5)c5c4oc4ccccc45)c([2H])c([2H])c32)c1[2H]]

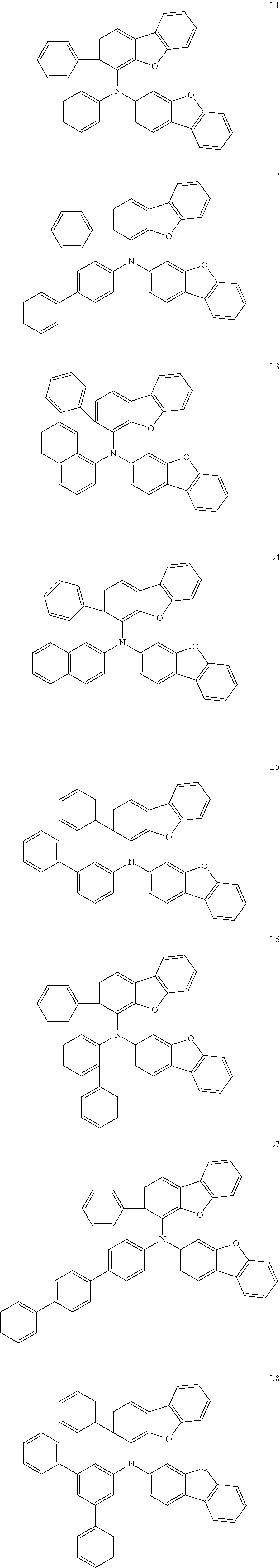

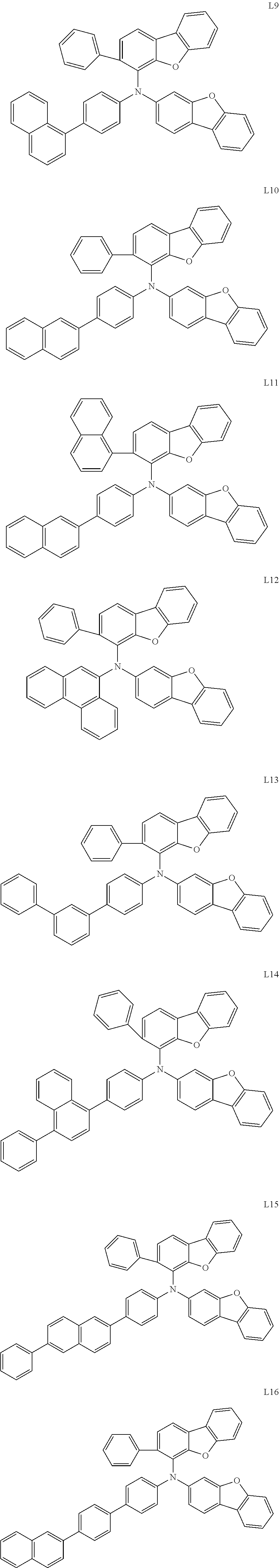

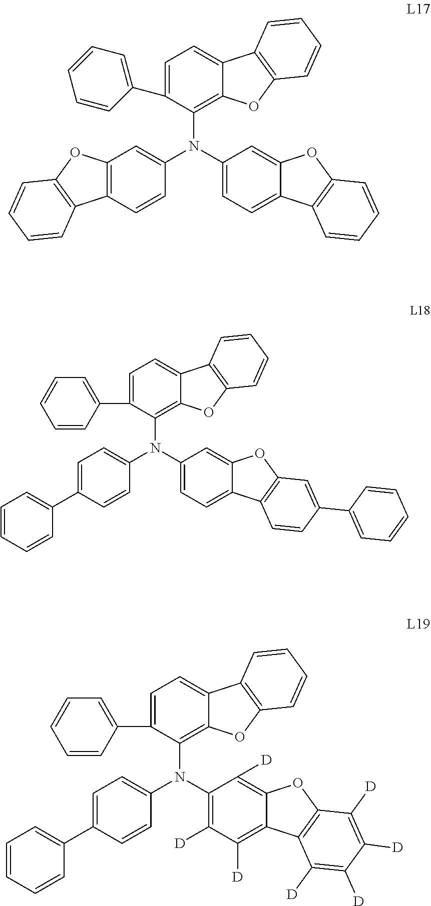 [SMILES: c1ccc(-c2ccc3c(oc4ccccc43)c2N(c2ccc3c(c2)oc2ccccc23)c2ccc3c(c2)oc2ccccc23)cc1 ; c1ccc(-c2ccc(N(c3ccc4c(c3)oc3cc(-c5ccccc5)ccc34)c3c(-c4ccccc4)ccc4c3oc3ccccc34)cc2)cc1 ; [2H]c1c([2H])c([2H])c2c(oc3c([2H])c(N(c4ccc(-c5ccccc5)cc4)c4c(-c5ccccc5)ccc5c4oc4ccccc45)c([2H])c([2H])c32)c1[2H]]

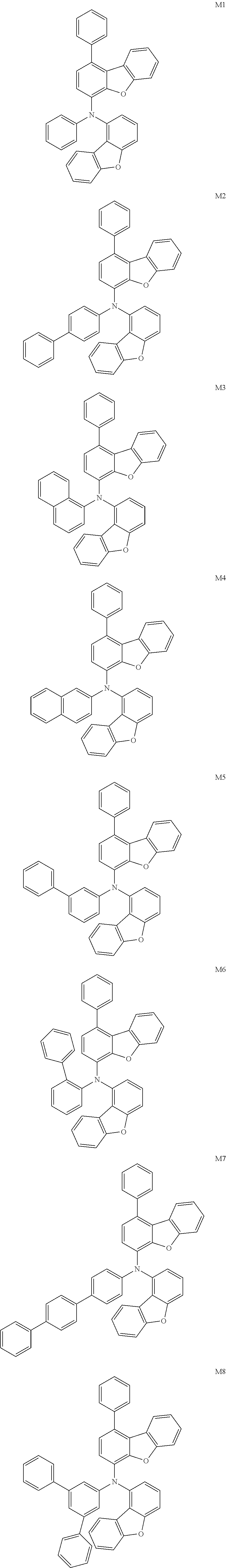

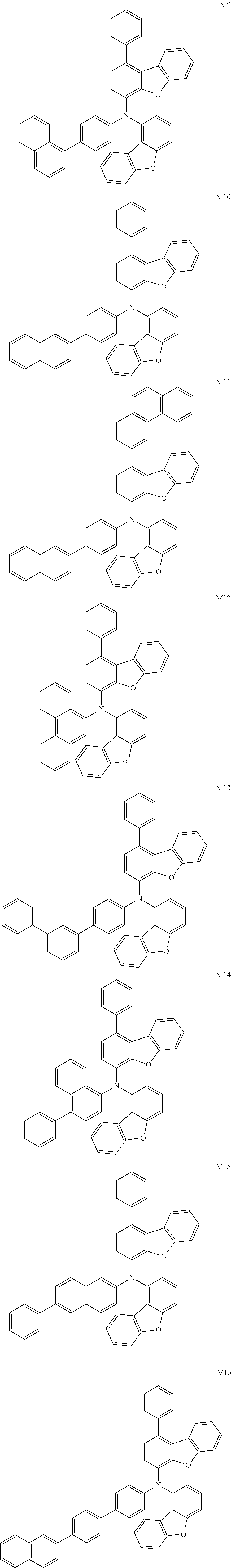

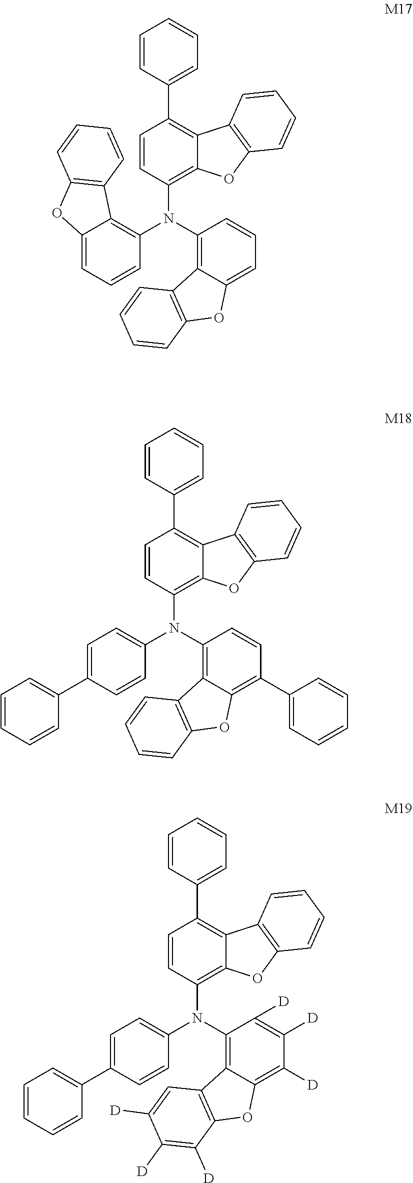 [SMILES: c1ccc(-c2ccc(N(c3cccc4oc5ccccc5c34)c3cccc4oc5ccccc5c34)c3oc4ccccc4c23)cc1 ; c1ccc(-c2ccc(N(c3ccc(-c4ccccc4)c4c3oc3ccccc34)c3ccc(-c4ccccc4)c4oc5ccccc5c34)cc2)cc1 ; [2H]c1cc2c(oc3c([2H])c([2H])c([2H])c(N(c4ccc(-c5ccccc5)cc4)c4ccc(-c5ccccc5)c5c4oc4ccccc45)c32)c([2H])c1[2H]]

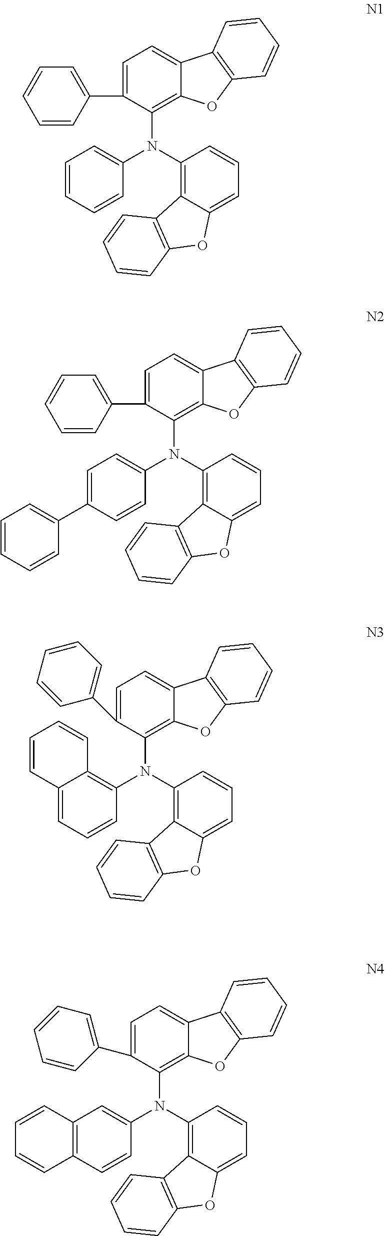 [SMILES: c1ccc(-c2ccc3c(oc4ccccc43)c2N(c2ccccc2)c2cccc3oc4ccccc4c23)cc1 ; c1ccc(-c2ccc(N(c3c(-c4ccccc4)ccc4c3oc3ccccc34)c3cccc4oc5ccccc5c34)cc2)cc1 ; c1ccc(-c2ccc3c(oc4ccccc43)c2N(c2cccc3ccccc23)c2cccc3oc4ccccc4c23)cc1 ; c1ccc(-c2ccc3c(oc4ccccc43)c2N(c2ccc3ccccc3c2)c2cccc3oc4ccccc4c23)cc1]

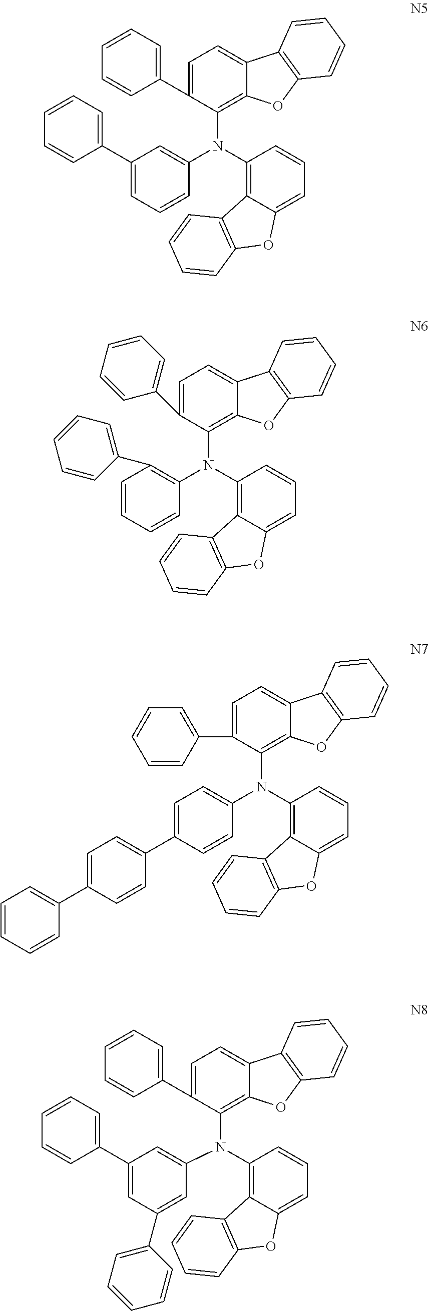 [SMILES: c1ccc(-c2cccc(N(c3c(-c4ccccc4)ccc4c3oc3ccccc34)c3cccc4oc5ccccc5c34)c2)cc1 ; c1ccc(-c2ccccc2N(c2c(-c3ccccc3)ccc3c2oc2ccccc23)c2cccc3oc4ccccc4c23)cc1 ; c1ccc(-c2ccc(-c3ccc(N(c4c(-c5ccccc5)ccc5c4oc4ccccc45)c4cccc5oc6ccccc6c45)cc3)cc2)cc1 ; c1ccc(-c2cc(-c3ccccc3)cc(N(c3c(-c4ccccc4)ccc4c3oc3ccccc34)c3cccc4oc5ccccc5c34)c2)cc1]

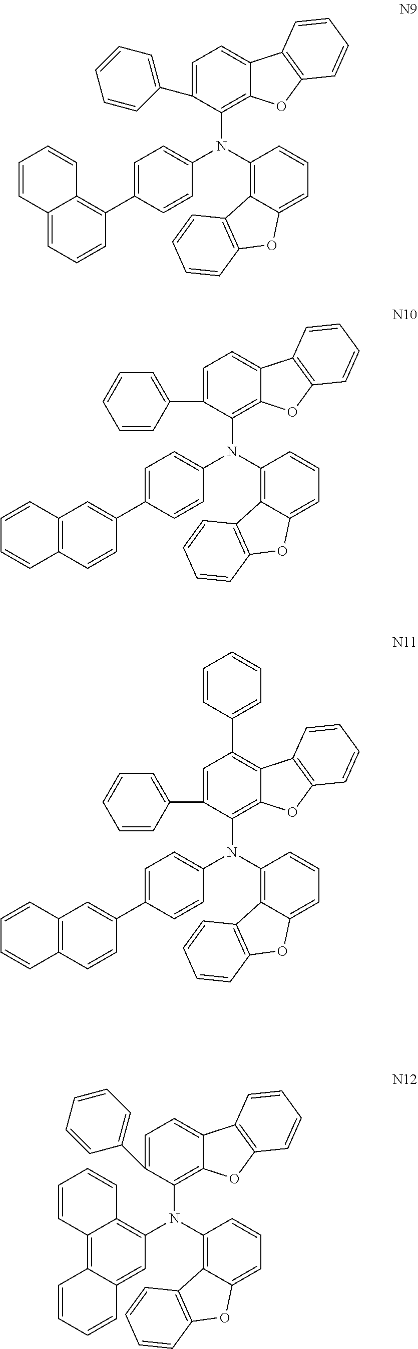 [SMILES: c1ccc(-c2ccc3c(oc4ccccc43)c2N(c2ccc(-c3cccc4ccccc34)cc2)c2cccc3oc4ccccc4c23)cc1 ; c1ccc(-c2ccc3c(oc4ccccc43)c2N(c2ccc(-c3ccc4ccccc4c3)cc2)c2cccc3oc4ccccc4c23)cc1 ; c1ccc(-c2cc(-c3ccccc3)c3c(oc4ccccc43)c2N(c2ccc(-c3ccc4ccccc4c3)cc2)c2cccc3oc4ccccc4c23)cc1 ; c1ccc(-c2ccc3c(oc4ccccc43)c2N(c2cc3ccccc3c3ccccc23)c2cccc3oc4ccccc4c23)cc1]

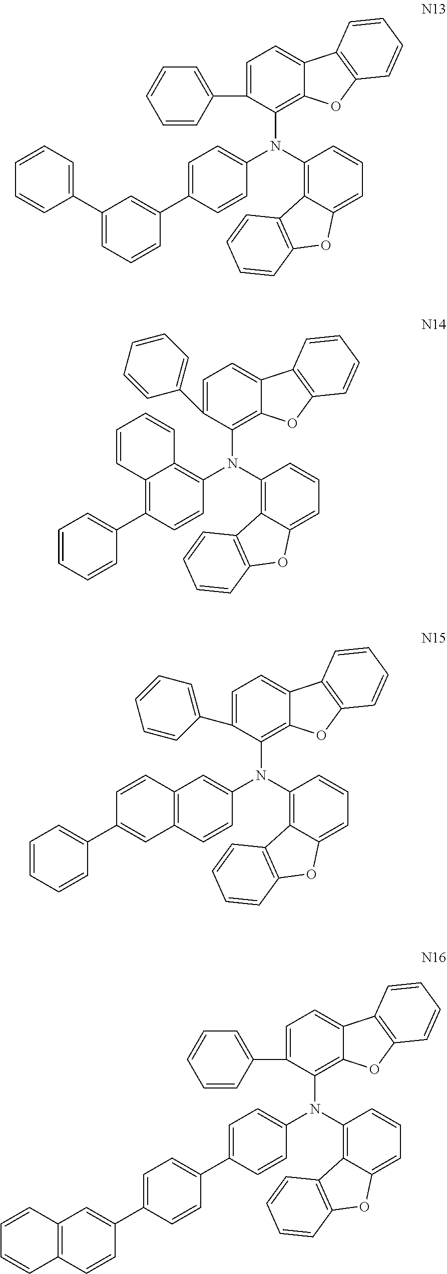 [SMILES: c1ccc(-c2cccc(-c3ccc(N(c4c(-c5ccccc5)ccc5c4oc4ccccc45)c4cccc5oc6ccccc6c45)cc3)c2)cc1 ; c1ccc(-c2ccc3c(oc4ccccc43)c2N(c2ccc(-c3ccccc3)c3ccccc23)c2cccc3oc4ccccc4c23)cc1 ; c1ccc(-c2ccc3cc(N(c4c(-c5ccccc5)ccc5c4oc4ccccc45)c4cccc5oc6ccccc6c45)ccc3c2)cc1 ; c1ccc(-c2ccc3c(oc4ccccc43)c2N(c2ccc(-c3ccc(-c4ccc5ccccc5c4)cc3)cc2)c2cccc3oc4ccccc4c23)cc1]

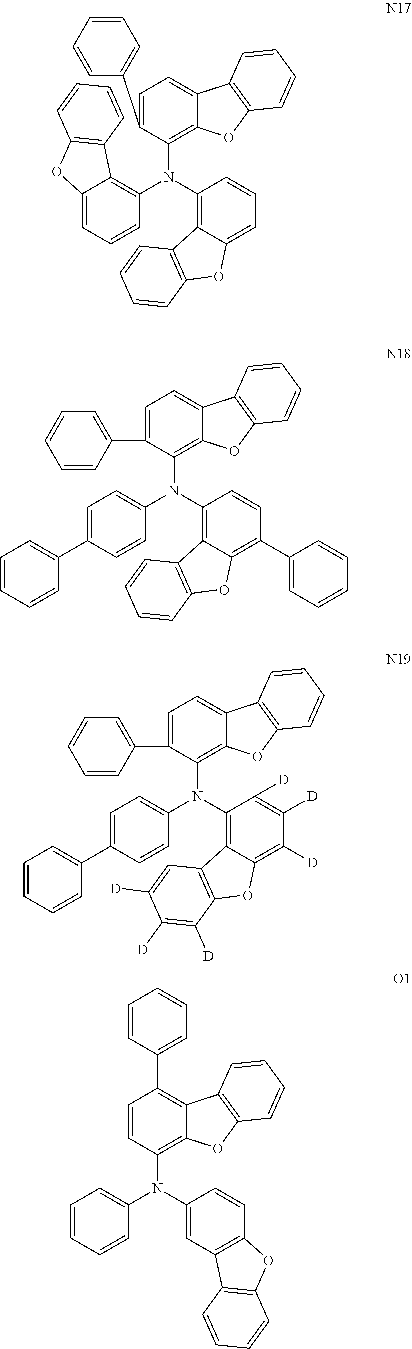

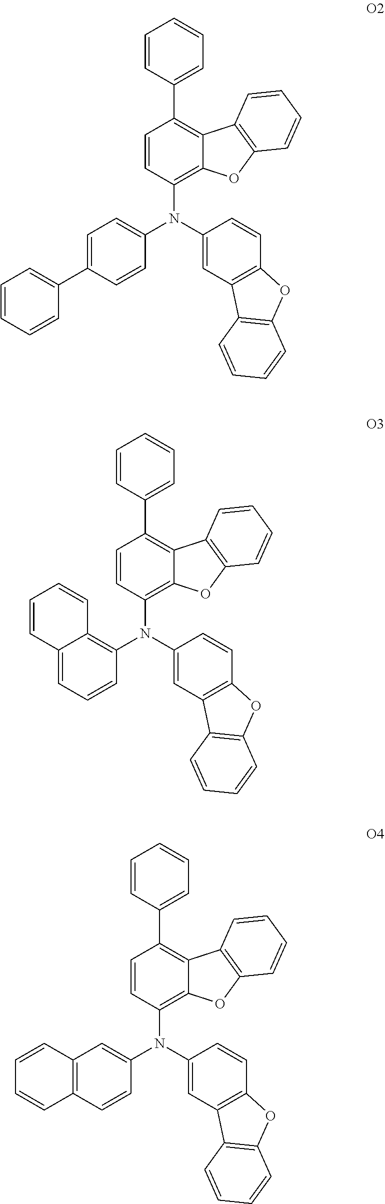 [SMILES: c1ccc(-c2ccc(N(c3ccc4oc5ccccc5c4c3)c3ccc(-c4ccccc4)c4c3oc3ccccc34)cc2)cc1 ; c1ccc(-c2ccc(N(c3ccc4oc5ccccc5c4c3)c3cccc4ccccc34)c3oc4ccccc4c23)cc1 ; c1ccc(-c2ccc(N(c3ccc4ccccc4c3)c3ccc4oc5ccccc5c4c3)c3oc4ccccc4c23)cc1]

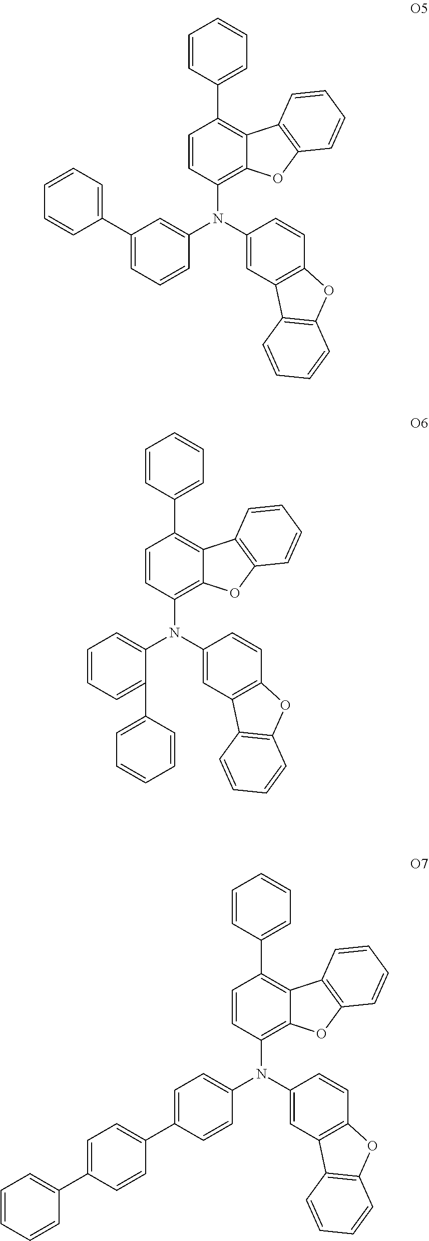 [SMILES: c1ccc(-c2cccc(N(c3ccc4oc5ccccc5c4c3)c3ccc(-c4ccccc4)c4c3oc3ccccc34)c2)cc1 ; c1ccc(-c2ccccc2N(c2ccc3oc4ccccc4c3c2)c2ccc(-c3ccccc3)c3c2oc2ccccc23)cc1 ; c1ccc(-c2ccc(-c3ccc(N(c4ccc5oc6ccccc6c5c4)c4ccc(-c5ccccc5)c5c4oc4ccccc45)cc3)cc2)cc1]

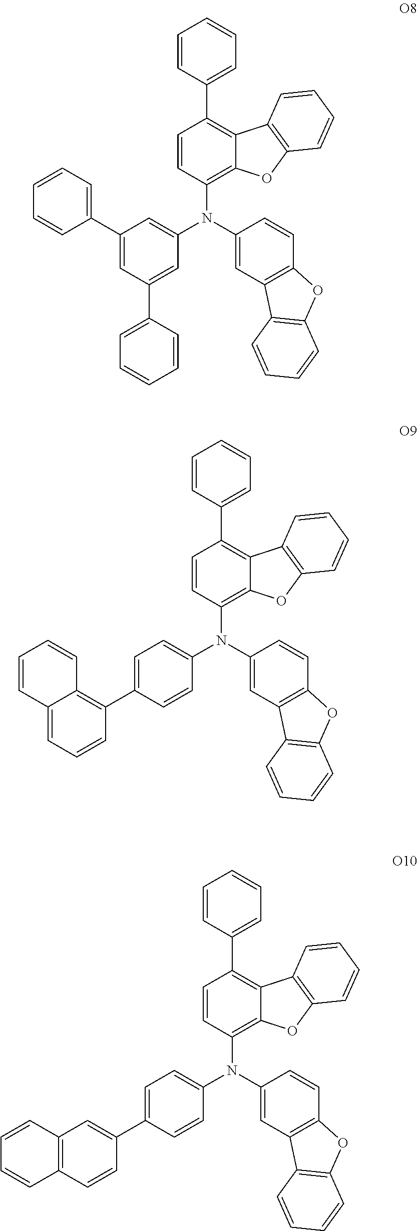 [SMILES: c1ccc(-c2cc(-c3ccccc3)cc(N(c3ccc4oc5ccccc5c4c3)c3ccc(-c4ccccc4)c4c3oc3ccccc34)c2)cc1 ; c1ccc(-c2ccc(N(c3ccc(-c4cccc5ccccc45)cc3)c3ccc4oc5ccccc5c4c3)c3oc4ccccc4c23)cc1 ; c1ccc(-c2ccc(N(c3ccc(-c4ccc5ccccc5c4)cc3)c3ccc4oc5ccccc5c4c3)c3oc4ccccc4c23)cc1]

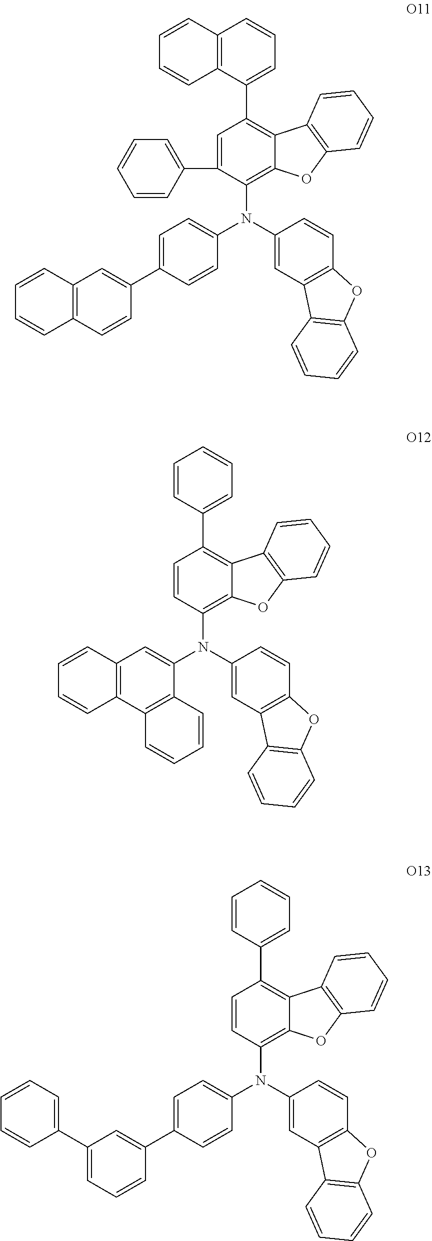 [SMILES: c1ccc(-c2cc(-c3cccc4ccccc34)c3c(oc4ccccc43)c2N(c2ccc(-c3ccc4ccccc4c3)cc2)c2ccc3oc4ccccc4c3c2)cc1 ; c1ccc(-c2ccc(N(c3ccc4oc5ccccc5c4c3)c3cc4ccccc4c4ccccc34)c3oc4ccccc4c23)cc1 ; c1ccc(-c2cccc(-c3ccc(N(c4ccc5oc6ccccc6c5c4)c4ccc(-c5ccccc5)c5c4oc4ccccc45)cc3)c2)cc1]

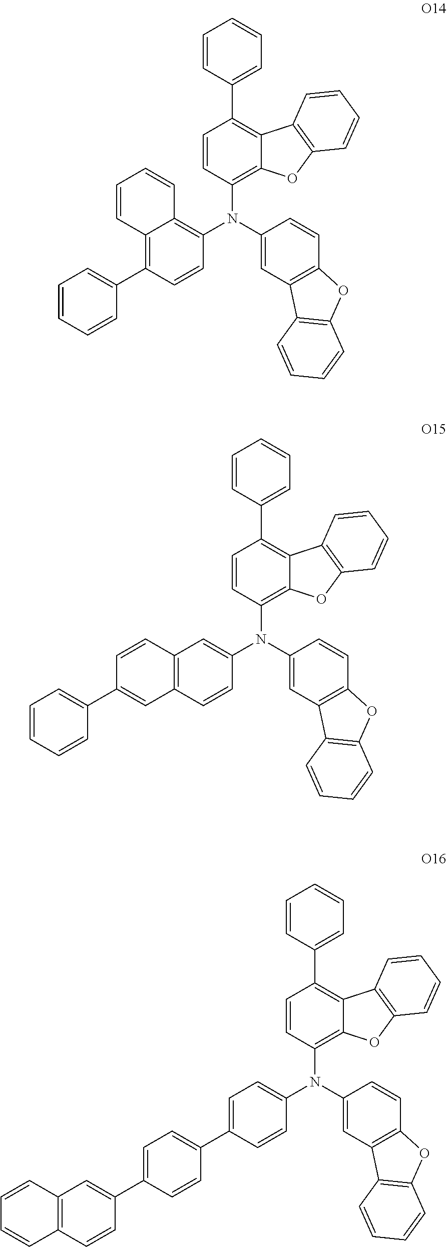 [SMILES: c1ccc(-c2ccc(N(c3ccc4oc5ccccc5c4c3)c3ccc(-c4ccccc4)c4c3oc3ccccc34)c3ccccc23)cc1 ; c1ccc(-c2ccc3cc(N(c4ccc5oc6ccccc6c5c4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc4oc5ccccc5c4c3)c3oc4ccccc4c23)cc1]

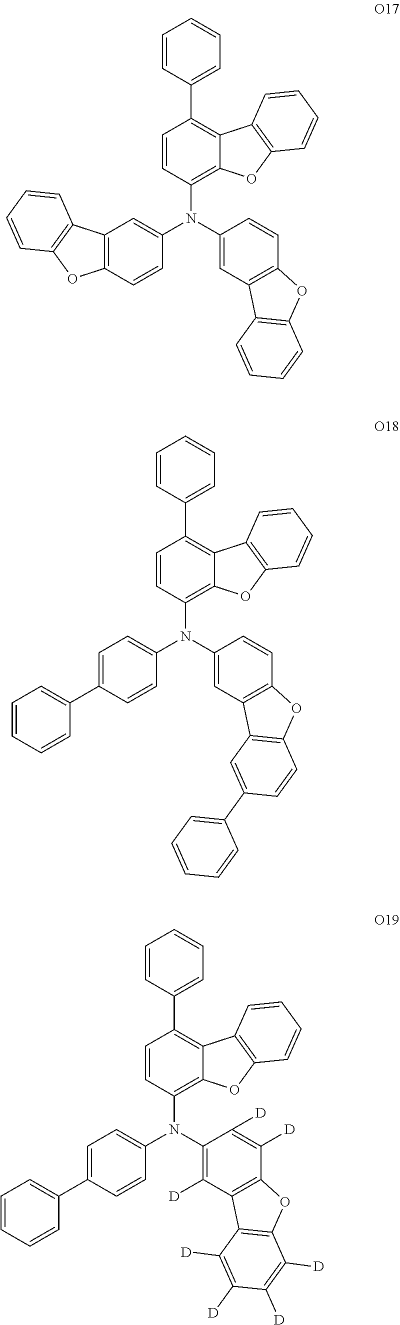 [SMILES: c1ccc(-c2ccc(N(c3ccc4oc5ccccc5c4c3)c3ccc4oc5ccccc5c4c3)c3oc4ccccc4c23)cc1 ; c1ccc(-c2ccc(N(c3ccc4oc5ccc(-c6ccccc6)cc5c4c3)c3ccc(-c4ccccc4)c4c3oc3ccccc34)cc2)cc1 ; [2H]c1c([2H])c([2H])c2c(oc3c([2H])c([2H])c(N(c4ccc(-c5ccccc5)cc4)c4ccc(-c5ccccc5)c5c4oc4ccccc45)c([2H])c32)c1[2H]]

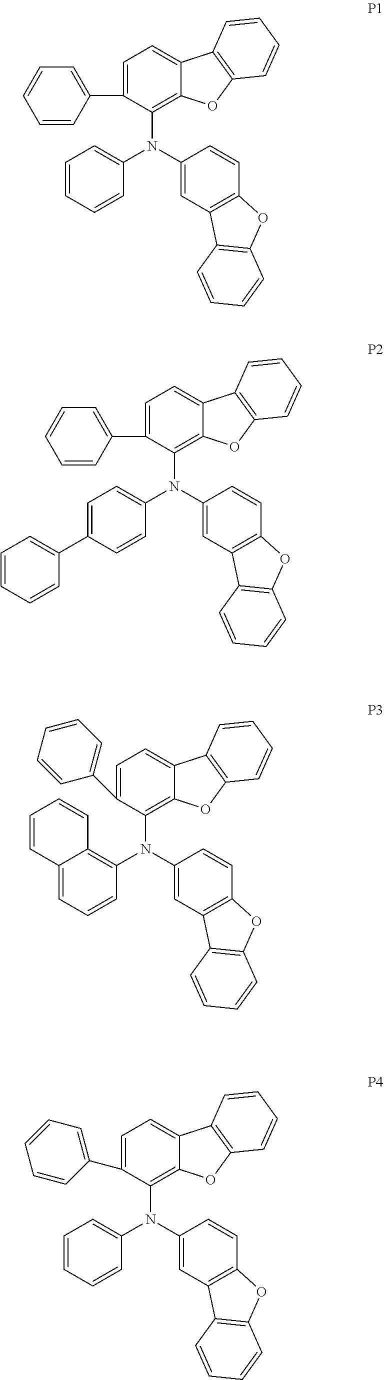 [SMILES: c1ccc(-c2ccc3c(oc4ccccc43)c2N(c2ccccc2)c2ccc3oc4ccccc4c3c2)cc1 ; c1ccc(-c2ccc(N(c3ccc4oc5ccccc5c4c3)c3c(-c4ccccc4)ccc4c3oc3ccccc34)cc2)cc1 ; c1ccc(-c2ccc3c(oc4ccccc43)c2N(c2ccc3oc4ccccc4c3c2)c2cccc3ccccc23)cc1 ; c1ccc(-c2ccc3c(oc4ccccc43)c2N(c2ccccc2)c2ccc3oc4ccccc4c3c2)cc1]

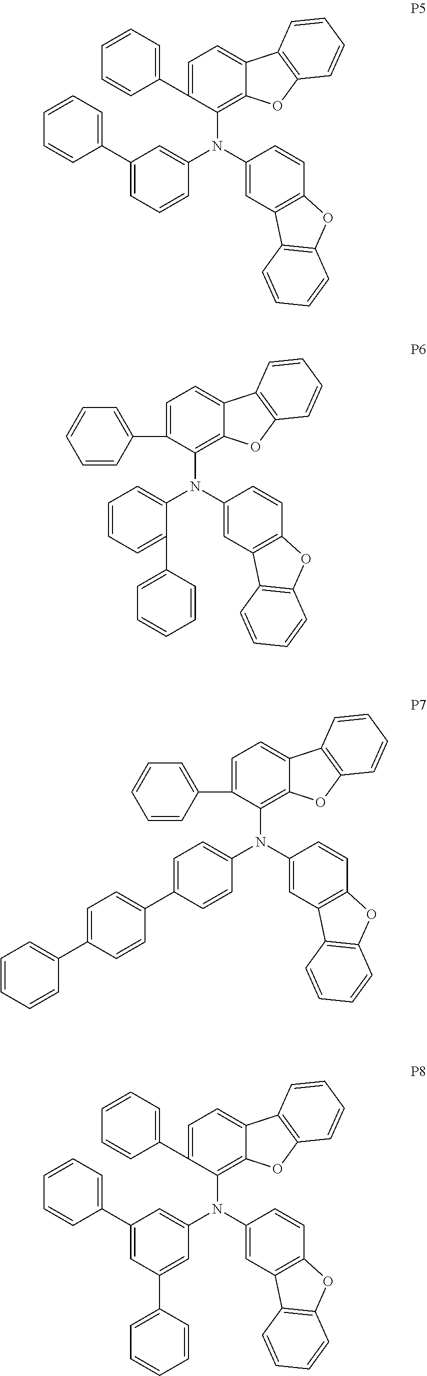 [SMILES: c1ccc(-c2cccc(N(c3ccc4oc5ccccc5c4c3)c3c(-c4ccccc4)ccc4c3oc3ccccc34)c2)cc1 ; c1ccc(-c2ccccc2N(c2ccc3oc4ccccc4c3c2)c2c(-c3ccccc3)ccc3c2oc2ccccc23)cc1 ; c1ccc(-c2ccc(-c3ccc(N(c4ccc5oc6ccccc6c5c4)c4c(-c5ccccc5)ccc5c4oc4ccccc45)cc3)cc2)cc1 ; c1ccc(-c2cc(-c3ccccc3)cc(N(c3ccc4oc5ccccc5c4c3)c3c(-c4ccccc4)ccc4c3oc3ccccc34)c2)cc1]

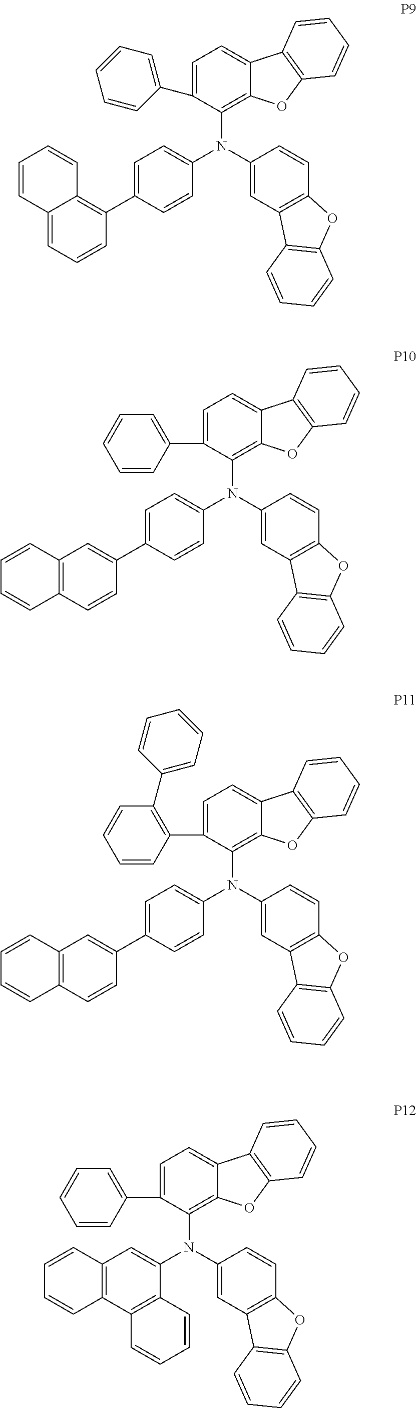 [SMILES: c1ccc(-c2ccc3c(oc4ccccc43)c2N(c2ccc(-c3cccc4ccccc34)cc2)c2ccc3oc4ccccc4c3c2)cc1 ; c1ccc(-c2ccc3c(oc4ccccc43)c2N(c2ccc(-c3ccc4ccccc4c3)cc2)c2ccc3oc4ccccc4c3c2)cc1 ; c1ccc(-c2ccccc2-c2ccc3c(oc4ccccc43)c2N(c2ccc(-c3ccc4ccccc4c3)cc2)c2ccc3oc4ccccc4c3c2)cc1 ; c1ccc(-c2ccc3c(oc4ccccc43)c2N(c2ccc3oc4ccccc4c3c2)c2cc3ccccc3c3ccccc23)cc1]

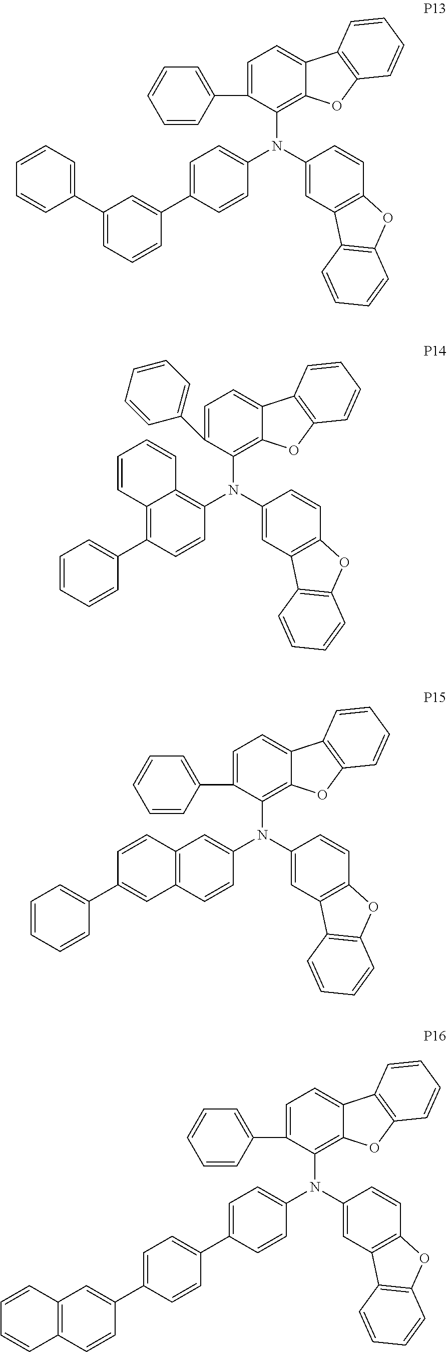 [SMILES: c1ccc(-c2cccc(-c3ccc(N(c4ccc5oc6ccccc6c5c4)c4c(-c5ccccc5)ccc5c4oc4ccccc45)cc3)c2)cc1 ; c1ccc(-c2ccc3c(oc4ccccc43)c2N(c2ccc3oc4ccccc4c3c2)c2ccc(-c3ccccc3)c3ccccc23)cc1 ; c1ccc(-c2ccc3cc(N(c4ccc5oc6ccccc6c5c4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc3oc4ccccc4c3c2)cc1]

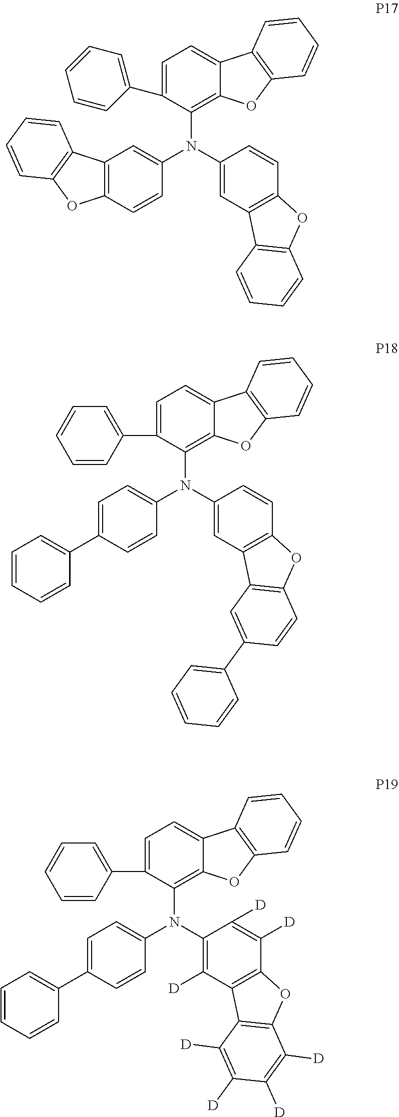 [SMILES: c1ccc(-c2ccc3c(oc4ccccc43)c2N(c2ccc3oc4ccccc4c3c2)c2ccc3oc4ccccc4c3c2)cc1 ; c1ccc(-c2ccc(N(c3ccc4oc5ccc(-c6ccccc6)cc5c4c3)c3c(-c4ccccc4)ccc4c3oc3ccccc34)cc2)cc1 ; [2H]c1c([2H])c([2H])c2c(oc3c([2H])c([2H])c(N(c4ccc(-c5ccccc5)cc4)c4c(-c5ccccc5)ccc5c4oc4ccccc45)c([2H])c32)c1[2H]]

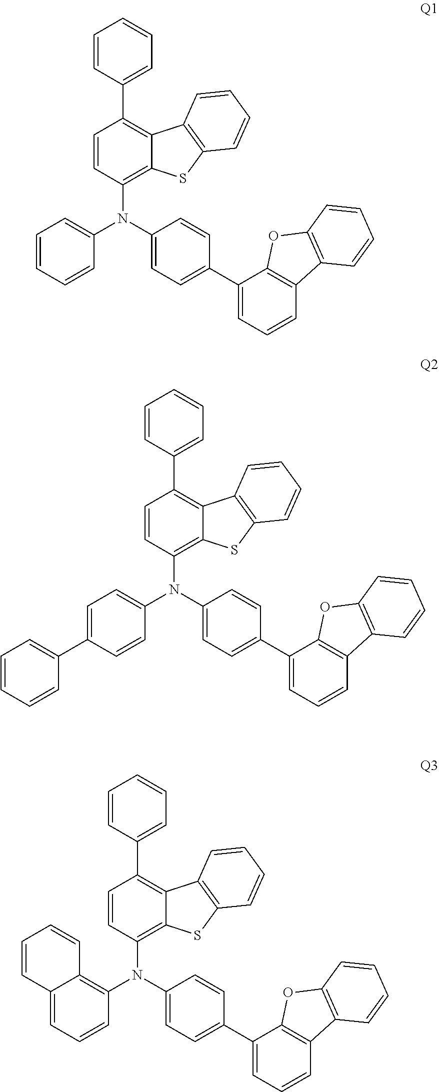 [SMILES: c1ccc(-c2ccc(N(c3ccccc3)c3ccc(-c4cccc5c4oc4ccccc45)cc3)c3sc4ccccc4c23)cc1 ; c1ccc(-c2ccc(N(c3ccc(-c4cccc5c4oc4ccccc45)cc3)c3ccc(-c4ccccc4)c4c3sc3ccccc34)cc2)cc1 ; c1ccc(-c2ccc(N(c3ccc(-c4cccc5c4oc4ccccc45)cc3)c3cccc4ccccc34)c3sc4ccccc4c23)cc1]

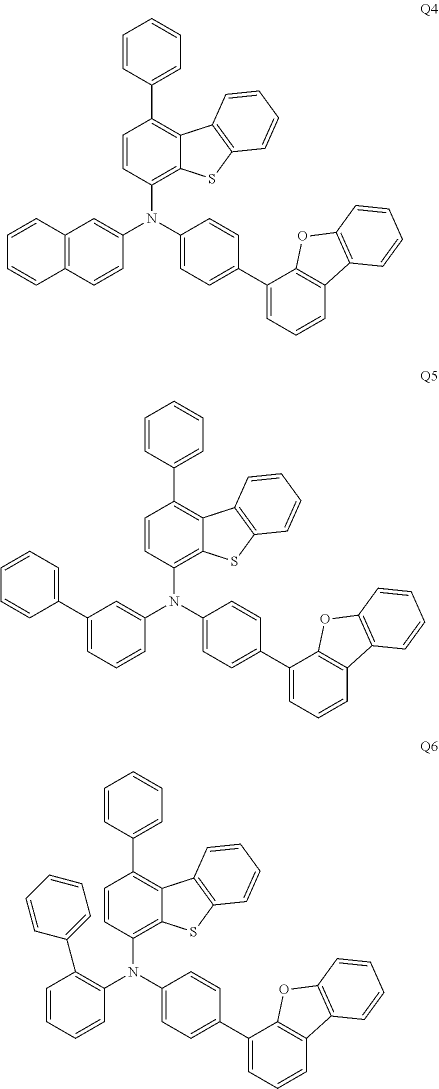 [SMILES: c1ccc(-c2ccc(N(c3ccc(-c4cccc5c4oc4ccccc45)cc3)c3ccc4ccccc4c3)c3sc4ccccc4c23)cc1 ; c1ccc(-c2cccc(N(c3ccc(-c4cccc5c4oc4ccccc45)cc3)c3ccc(-c4ccccc4)c4c3sc3ccccc34)c2)cc1 ; c1ccc(-c2ccccc2N(c2ccc(-c3cccc4c3oc3ccccc34)cc2)c2ccc(-c3ccccc3)c3c2sc2ccccc23)cc1]

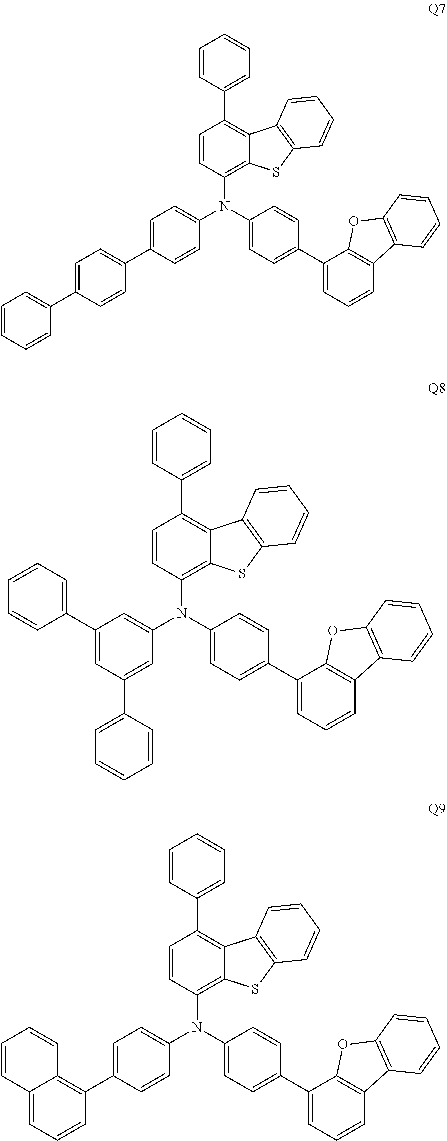 [SMILES: c1ccc(-c2ccc(-c3ccc(N(c4ccc(-c5cccc6c5oc5ccccc56)cc4)c4ccc(-c5ccccc5)c5c4sc4ccccc45)cc3)cc2)cc1 ; c1ccc(-c2cc(-c3ccccc3)cc(N(c3ccc(-c4cccc5c4oc4ccccc45)cc3)c3ccc(-c4ccccc4)c4c3sc3ccccc34)c2)cc1 ; c1ccc(-c2ccc(N(c3ccc(-c4cccc5ccccc45)cc3)c3ccc(-c4cccc5c4oc4ccccc45)cc3)c3sc4ccccc4c23)cc1]

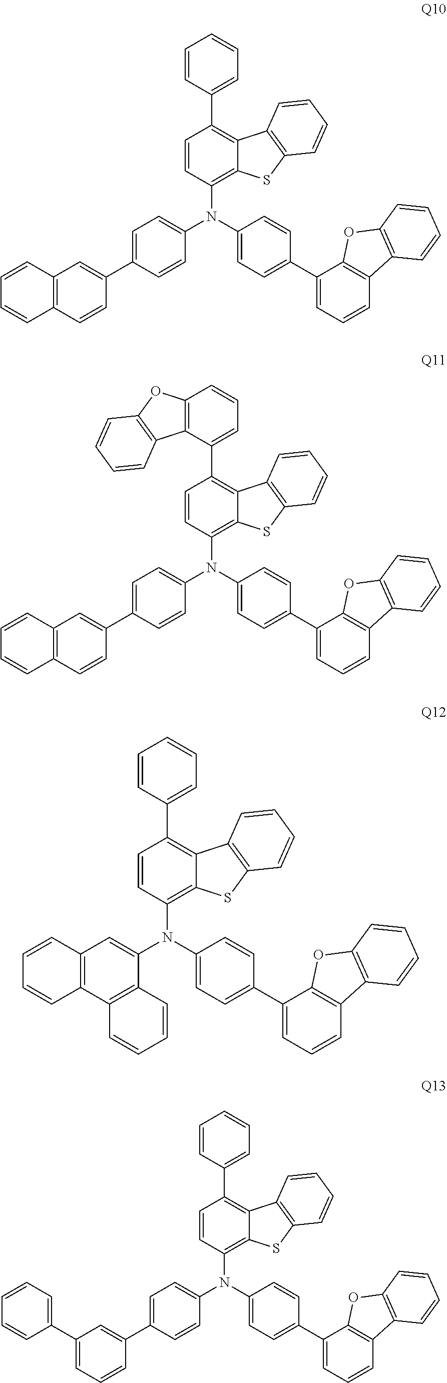 [SMILES: c1ccc(-c2ccc(N(c3ccc(-c4ccc5ccccc5c4)cc3)c3ccc(-c4cccc5c4oc4ccccc45)cc3)c3sc4ccccc4c23)cc1 ; c1ccc2cc(-c3ccc(N(c4ccc(-c5cccc6c5oc5ccccc56)cc4)c4ccc(-c5cccc6oc7ccccc7c56)c5c4sc4ccccc45)cc3)ccc2c1 ; c1ccc(-c2ccc(N(c3ccc(-c4cccc5c4oc4ccccc45)cc3)c3cc4ccccc4c4ccccc34)c3sc4ccccc4c23)cc1 ; c1ccc(-c2cccc(-c3ccc(N(c4ccc(-c5cccc6c5oc5ccccc56)cc4)c4ccc(-c5ccccc5)c5c4sc4ccccc45)cc3)c2)cc1]

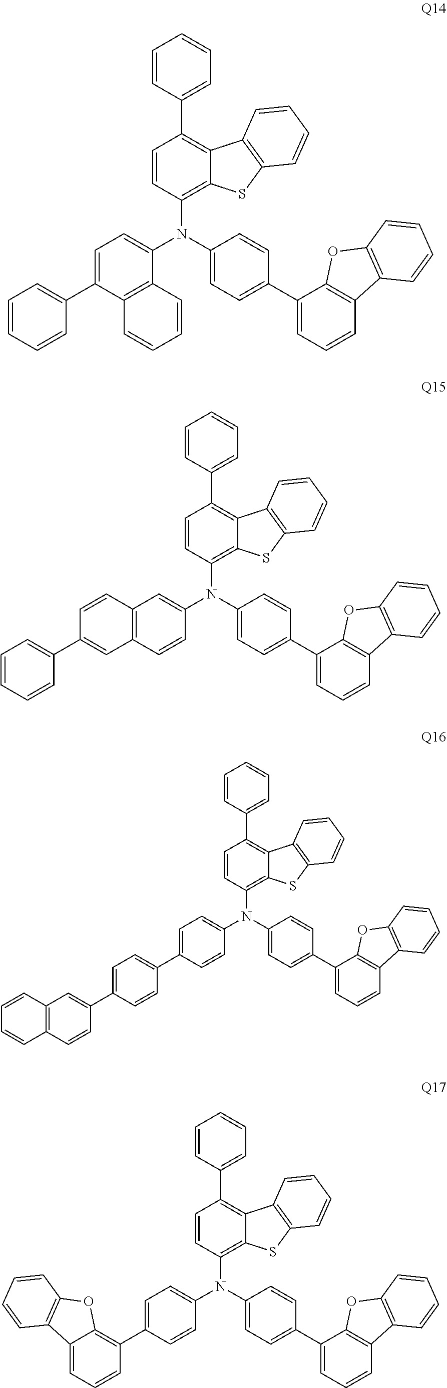 [SMILES: c1ccc(-c2ccc(N(c3ccc(-c4cccc5c4oc4ccccc45)cc3)c3ccc(-c4ccccc4)c4c3sc3ccccc34)c3ccccc23)cc1 ; c1ccc(-c2ccc3cc(N(c4ccc(-c5cccc6c5oc5ccccc56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4cccc5c4oc4ccccc45)cc3)c3sc4ccccc4c23)cc1 ; c1ccc(-c2ccc(N(c3ccc(-c4cccc5c4oc4ccccc45)cc3)c3ccc(-c4cccc5c4oc4ccccc45)cc3)c3sc4ccccc4c23)cc1]

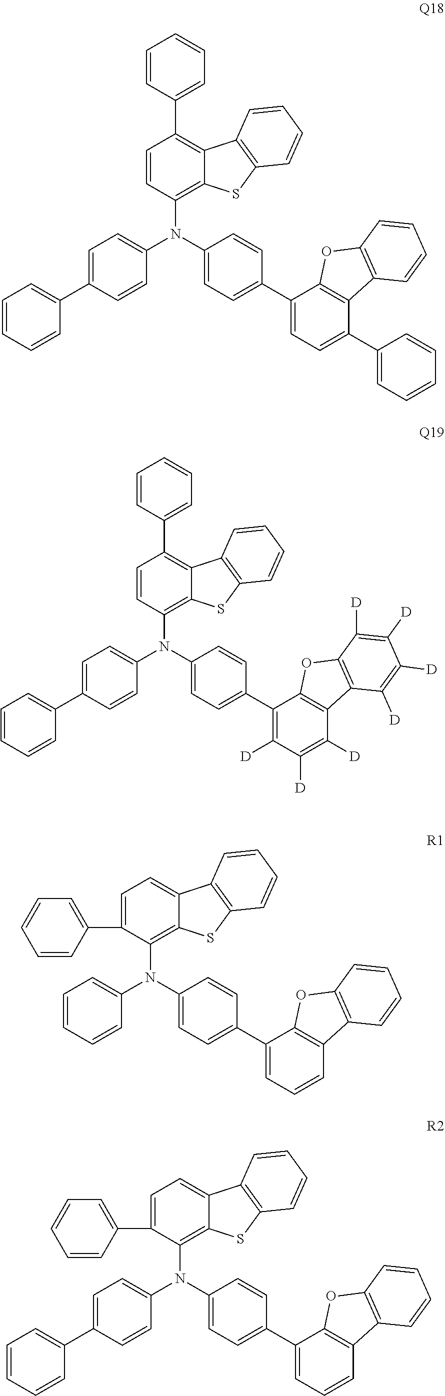 [SMILES: c1ccc(-c2ccc(N(c3ccc(-c4ccc(-c5ccccc5)c5c4oc4ccccc45)cc3)c3ccc(-c4ccccc4)c4c3sc3ccccc34)cc2)cc1 ; [2H]c1c([2H])c([2H])c2c(oc3c(-c4ccc(N(c5ccc(-c6ccccc6)cc5)c5ccc(-c6ccccc6)c6c5sc5ccccc56)cc4)c([2H])c([2H])c([2H])c32)c1[2H] ; c1ccc(-c2ccc3c(sc4ccccc43)c2N(c2ccccc2)c2ccc(-c3cccc4c3oc3ccccc34)cc2)cc1 ; c1ccc(-c2ccc(N(c3ccc(-c4cccc5c4oc4ccccc45)cc3)c3c(-c4ccccc4)ccc4c3sc3ccccc34)cc2)cc1]

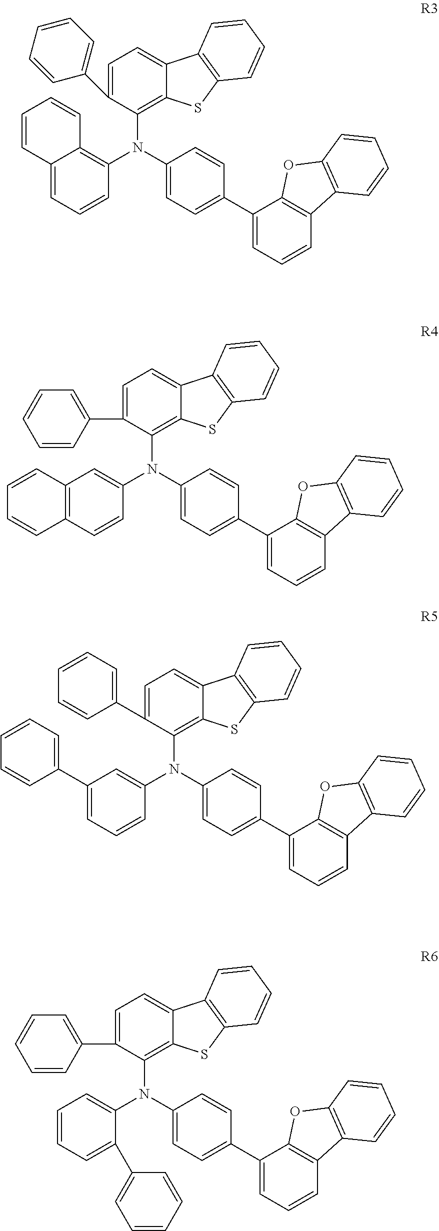 [SMILES: c1ccc(-c2ccc3c(sc4ccccc43)c2N(c2ccc(-c3cccc4c3oc3ccccc34)cc2)c2cccc3ccccc23)cc1 ; c1ccc(-c2ccc3c(sc4ccccc43)c2N(c2ccc(-c3cccc4c3oc3ccccc34)cc2)c2ccc3ccccc3c2)cc1 ; c1ccc(-c2cccc(N(c3ccc(-c4cccc5c4oc4ccccc45)cc3)c3c(-c4ccccc4)ccc4c3sc3ccccc34)c2)cc1 ; c1ccc(-c2ccccc2N(c2ccc(-c3cccc4c3oc3ccccc34)cc2)c2c(-c3ccccc3)ccc3c2sc2ccccc23)cc1]

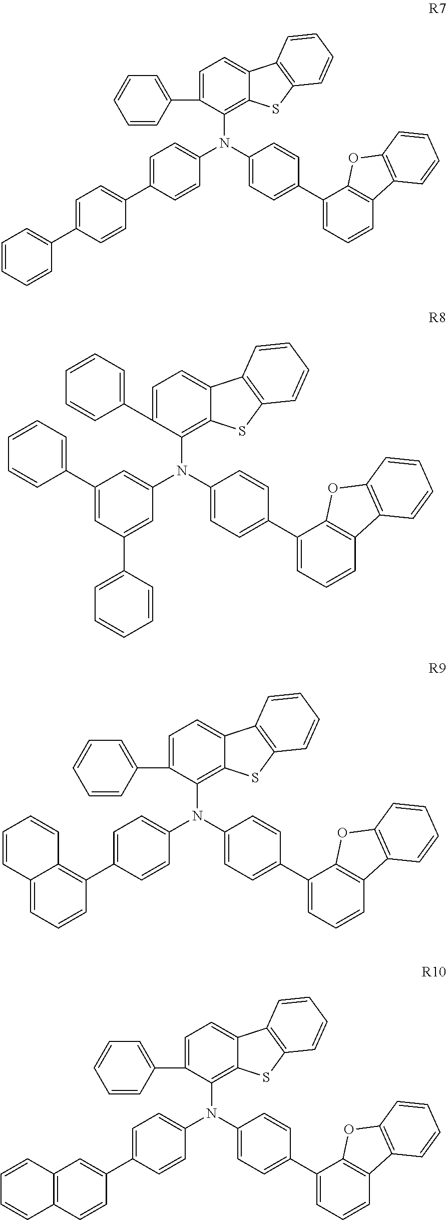 [SMILES: c1ccc(-c2ccc(-c3ccc(N(c4ccc(-c5cccc6c5oc5ccccc56)cc4)c4c(-c5ccccc5)ccc5c4sc4ccccc45)cc3)cc2)cc1 ; c1ccc(-c2cc(-c3ccccc3)cc(N(c3ccc(-c4cccc5c4oc4ccccc45)cc3)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2ccc(-c3cccc4c3oc3ccccc34)cc2)cc1 ; c1ccc(-c2ccc3c(sc4ccccc43)c2N(c2ccc(-c3ccc4ccccc4c3)cc2)c2ccc(-c3cccc4c3oc3ccccc34)cc2)cc1]

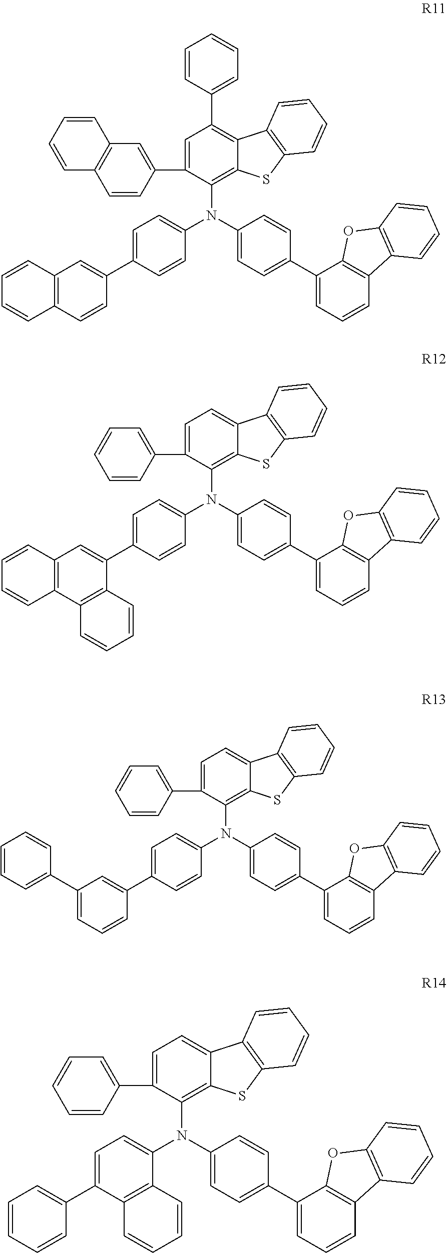 [SMILES: c1ccc(-c2cc(-c3ccc4ccccc4c3)c(N(c3ccc(-c4ccc5ccccc5c4)cc3)c3ccc(-c4cccc5c4oc4ccccc45)cc3)c3sc4ccccc4c23)cc1 ; c1ccc(-c2ccc3c(sc4ccccc43)c2N(c2ccc(-c3cc4ccccc4c4ccccc34)cc2)c2ccc(-c3cccc4c3oc3ccccc34)cc2)cc1 ; c1ccc(-c2cccc(-c3ccc(N(c4ccc(-c5cccc6c5oc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3cccc4c3oc3ccccc34)cc2)c2ccc(-c3ccccc3)c3ccccc23)cc1]

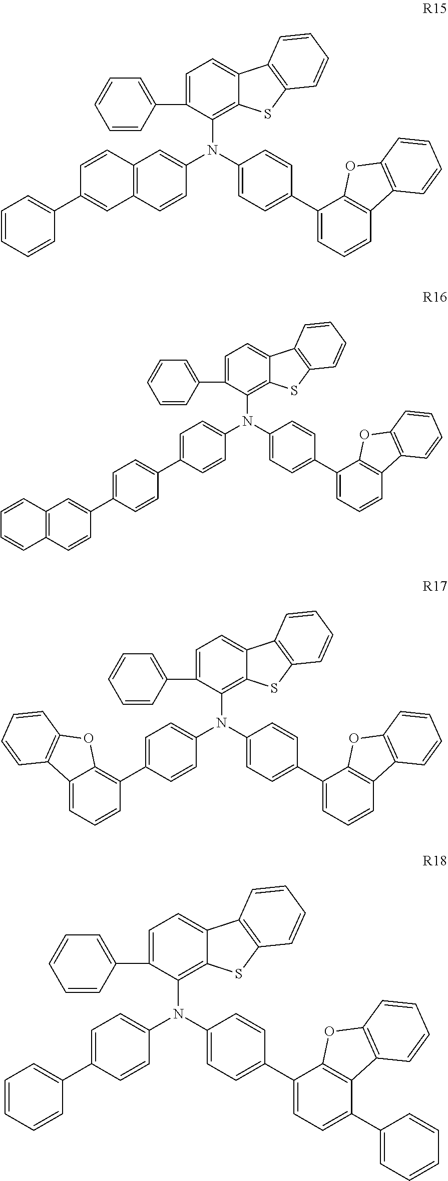 [SMILES: c1ccc(-c2ccc3cc(N(c4ccc(-c5cccc6c5oc5ccccc56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3cccc4c3oc3ccccc34)cc2)cc1 ; c1ccc(-c2ccc3c(sc4ccccc43)c2N(c2ccc(-c3cccc4c3oc3ccccc34)cc2)c2ccc(-c3cccc4c3oc3ccccc34)cc2)cc1 ; c1ccc(-c2ccc(N(c3ccc(-c4ccc(-c5ccccc5)c5c4oc4ccccc45)cc3)c3c(-c4ccccc4)ccc4c3sc3ccccc34)cc2)cc1]

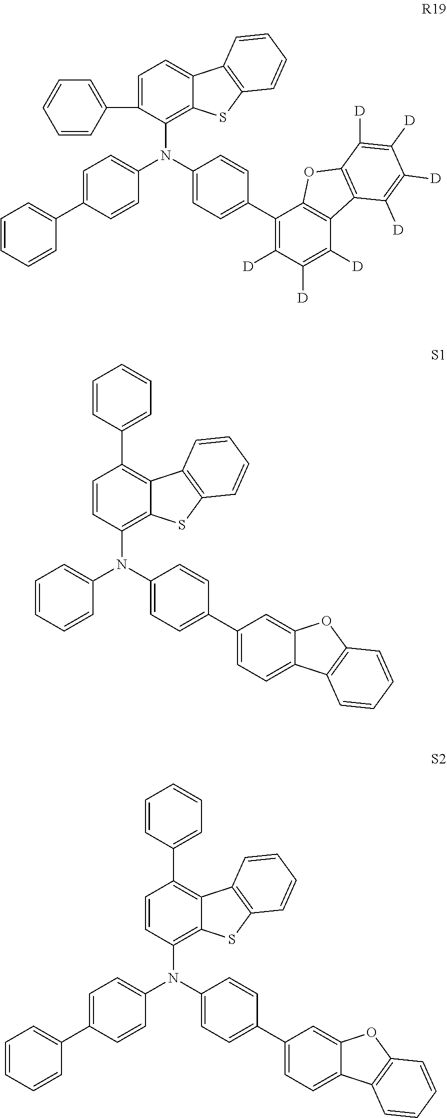 [SMILES: [2H]c1c([2H])c([2H])c2c(oc3c(-c4ccc(N(c5ccc(-c6ccccc6)cc5)c5c(-c6ccccc6)ccc6c5sc5ccccc56)cc4)c([2H])c([2H])c([2H])c32)c1[2H] ; c1ccc(-c2ccc(N(c3ccccc3)c3ccc(-c4ccc5c(c4)oc4ccccc45)cc3)c3sc4ccccc4c23)cc1 ; c1ccc(-c2ccc(N(c3ccc(-c4ccc5c(c4)oc4ccccc45)cc3)c3ccc(-c4ccccc4)c4c3sc3ccccc34)cc2)cc1]

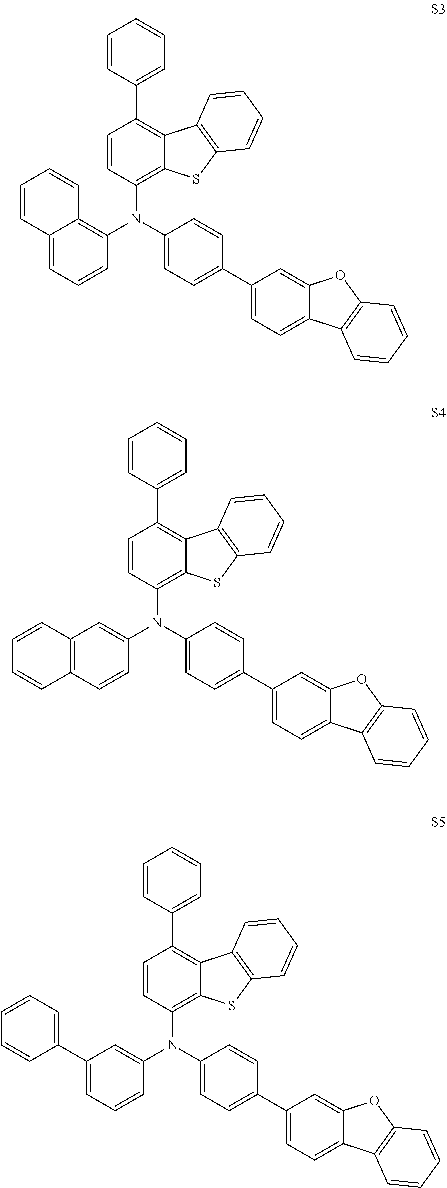 [SMILES: c1ccc(-c2ccc(N(c3ccc(-c4ccc5c(c4)oc4ccccc45)cc3)c3cccc4ccccc34)c3sc4ccccc4c23)cc1 ; c1ccc(-c2ccc(N(c3ccc(-c4ccc5c(c4)oc4ccccc45)cc3)c3ccc4ccccc4c3)c3sc4ccccc4c23)cc1 ; c1ccc(-c2cccc(N(c3ccc(-c4ccc5c(c4)oc4ccccc45)cc3)c3ccc(-c4ccccc4)c4c3sc3ccccc34)c2)cc1]

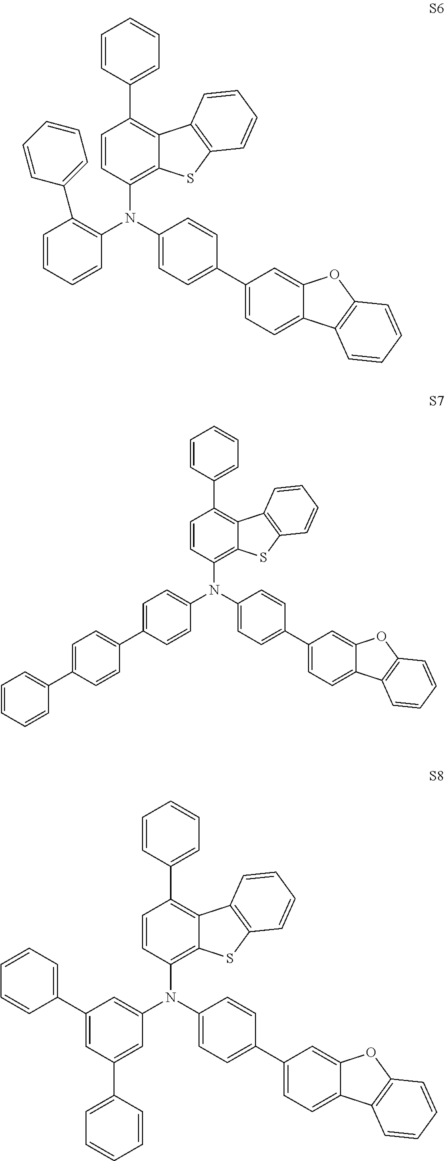 [SMILES: c1ccc(-c2ccccc2N(c2ccc(-c3ccc4c(c3)oc3ccccc34)cc2)c2ccc(-c3ccccc3)c3c2sc2ccccc23)cc1 ; c1ccc(-c2ccc(-c3ccc(N(c4ccc(-c5ccc6c(c5)oc5ccccc56)cc4)c4ccc(-c5ccccc5)c5c4sc4ccccc45)cc3)cc2)cc1 ; c1ccc(-c2cc(-c3ccccc3)cc(N(c3ccc(-c4ccc5c(c4)oc4ccccc45)cc3)c3ccc(-c4ccccc4)c4c3sc3ccccc34)c2)cc1]

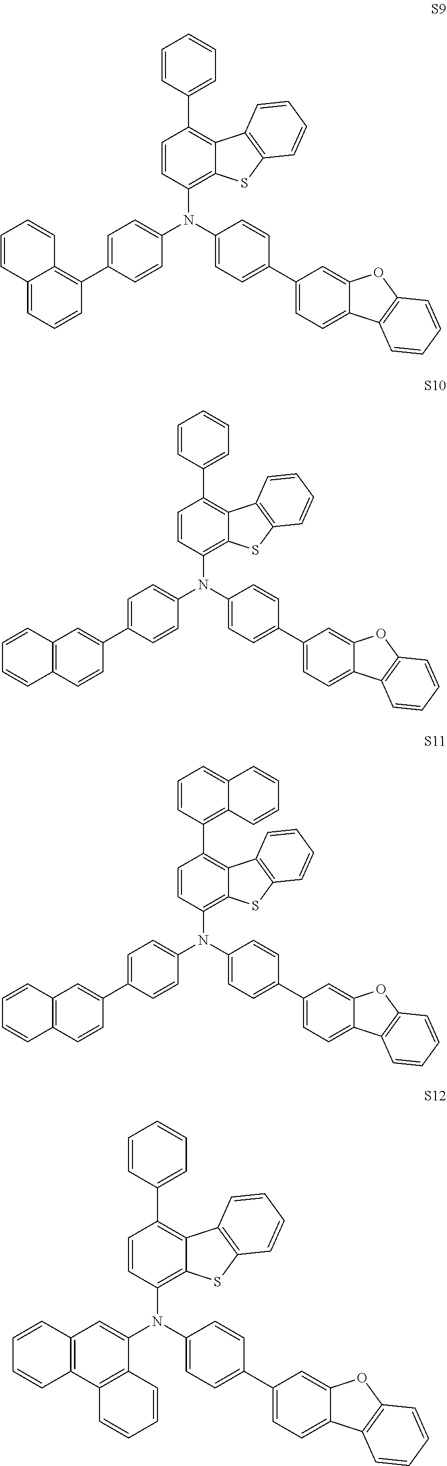 [SMILES: c1ccc(-c2ccc(N(c3ccc(-c4ccc5c(c4)oc4ccccc45)cc3)c3ccc(-c4cccc5ccccc45)cc3)c3sc4ccccc4c23)cc1 ; c1ccc(-c2ccc(N(c3ccc(-c4ccc5ccccc5c4)cc3)c3ccc(-c4ccc5c(c4)oc4ccccc45)cc3)c3sc4ccccc4c23)cc1 ; c1ccc2cc(-c3ccc(N(c4ccc(-c5ccc6c(c5)oc5ccccc56)cc4)c4ccc(-c5cccc6ccccc56)c5c4sc4ccccc45)cc3)ccc2c1 ; c1ccc(-c2ccc(N(c3ccc(-c4ccc5c(c4)oc4ccccc45)cc3)c3cc4ccccc4c4ccccc34)c3sc4ccccc4c23)cc1]

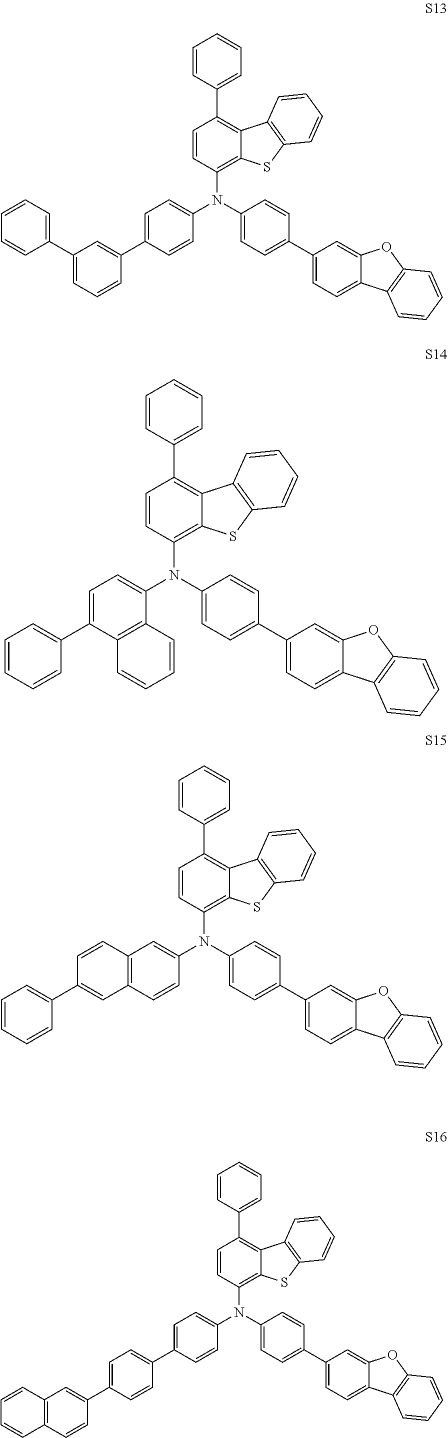 [SMILES: c1ccc(-c2cccc(-c3ccc(N(c4ccc(-c5ccc6c(c5)oc5ccccc56)cc4)c4ccc(-c5ccccc5)c5c4sc4ccccc45)cc3)c2)cc1 ; c1ccc(-c2ccc(N(c3ccc(-c4ccc5c(c4)oc4ccccc45)cc3)c3ccc(-c4ccccc4)c4c3sc3ccccc34)c3ccccc23)cc1 ; c1ccc(-c2ccc3cc(N(c4ccc(-c5ccc6c(c5)oc5ccccc56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4ccc5c(c4)oc4ccccc45)cc3)c3sc4ccccc4c23)cc1]

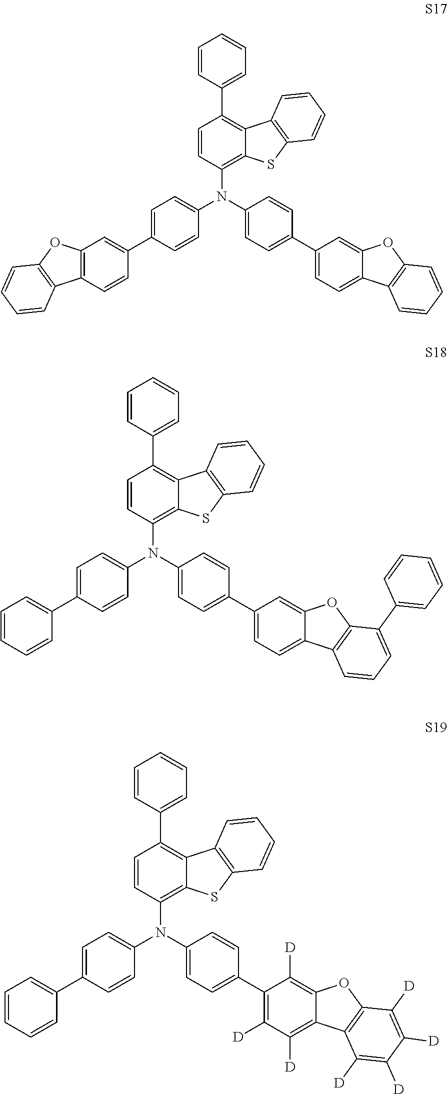 [SMILES: c1ccc(-c2ccc(N(c3ccc(-c4ccc5c(c4)oc4ccccc45)cc3)c3ccc(-c4ccc5c(c4)oc4ccccc45)cc3)c3sc4ccccc4c23)cc1 ; c1ccc(-c2ccc(N(c3ccc(-c4ccc5c(c4)oc4c(-c6ccccc6)cccc45)cc3)c3ccc(-c4ccccc4)c4c3sc3ccccc34)cc2)cc1 ; [2H]c1c([2H])c([2H])c2c(oc3c([2H])c(-c4ccc(N(c5ccc(-c6ccccc6)cc5)c5ccc(-c6ccccc6)c6c5sc5ccccc56)cc4)c([2H])c([2H])c32)c1[2H]]

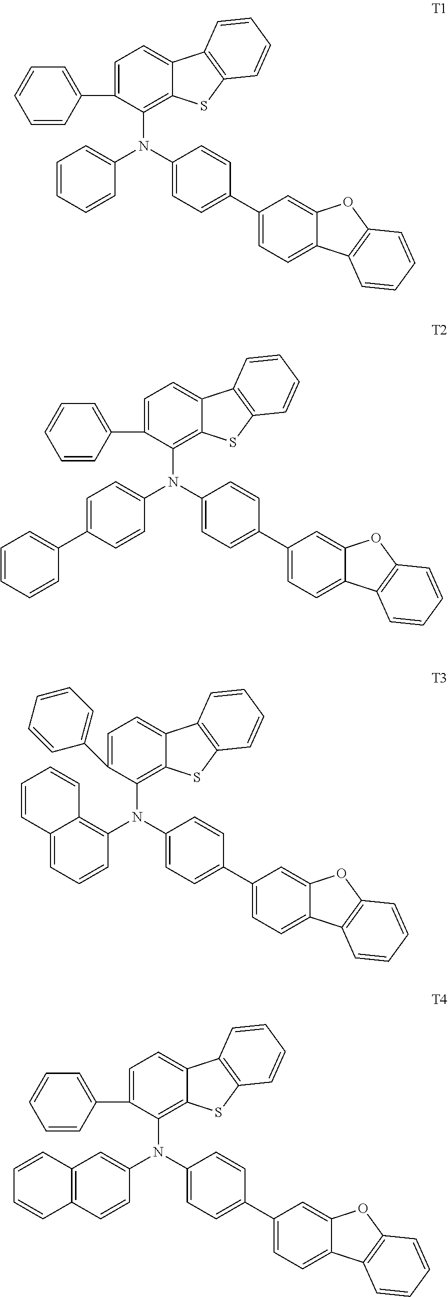 [SMILES: c1ccc(-c2ccc3c(sc4ccccc43)c2N(c2ccccc2)c2ccc(-c3ccc4c(c3)oc3ccccc34)cc2)cc1 ; c1ccc(-c2ccc(N(c3ccc(-c4ccc5c(c4)oc4ccccc45)cc3)c3c(-c4ccccc4)ccc4c3sc3ccccc34)cc2)cc1 ; c1ccc(-c2ccc3c(sc4ccccc43)c2N(c2ccc(-c3ccc4c(c3)oc3ccccc34)cc2)c2cccc3ccccc23)cc1 ; c1ccc(-c2ccc3c(sc4ccccc43)c2N(c2ccc(-c3ccc4c(c3)oc3ccccc34)cc2)c2ccc3ccccc3c2)cc1]

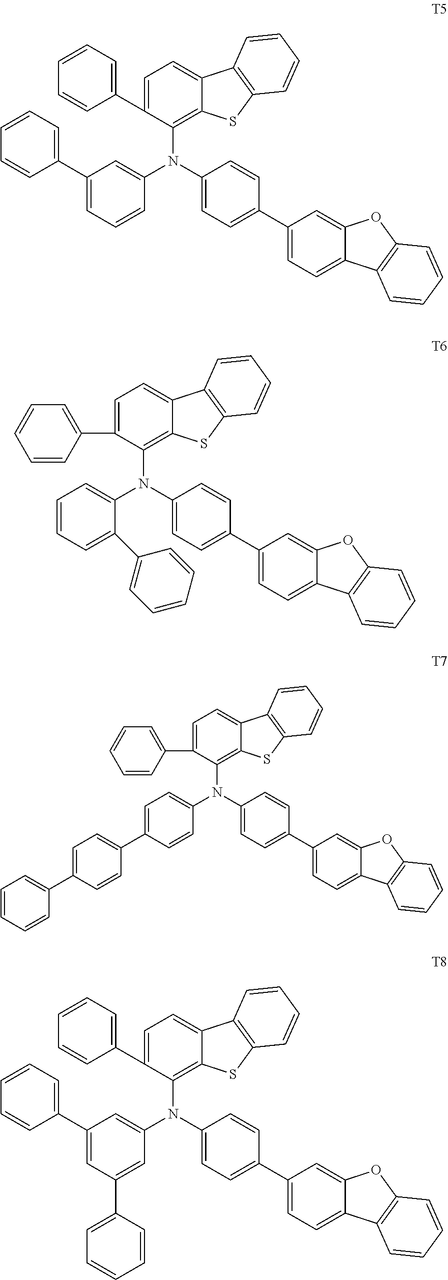 [SMILES: c1ccc(-c2cccc(N(c3ccc(-c4ccc5c(c4)oc4ccccc45)cc3)c3c(-c4ccccc4)ccc4c3sc3ccccc34)c2)cc1 ; c1ccc(-c2ccccc2N(c2ccc(-c3ccc4c(c3)oc3ccccc34)cc2)c2c(-c3ccccc3)ccc3c2sc2ccccc23)cc1 ; c1ccc(-c2ccc(-c3ccc(N(c4ccc(-c5ccc6c(c5)oc5ccccc56)cc4)c4c(-c5ccccc5)ccc5c4sc4ccccc45)cc3)cc2)cc1 ; c1ccc(-c2cc(-c3ccccc3)cc(N(c3ccc(-c4ccc5c(c4)oc4ccccc45)cc3)c3c(-c4ccccc4)ccc4c3sc3ccccc34)c2)cc1]

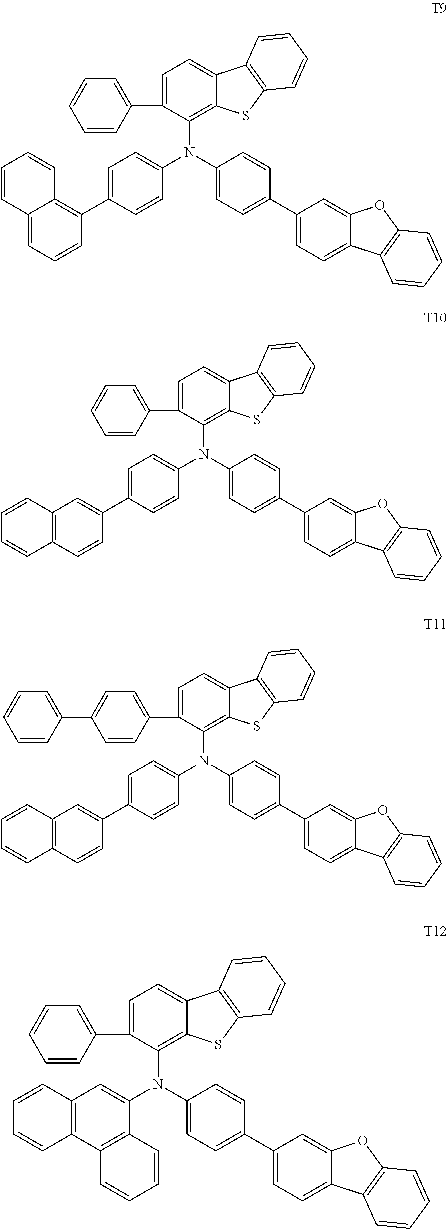 [SMILES: c1ccc(-c2ccc3c(sc4ccccc43)c2N(c2ccc(-c3ccc4c(c3)oc3ccccc34)cc2)c2ccc(-c3cccc4ccccc34)cc2)cc1 ; c1ccc(-c2ccc3c(sc4ccccc43)c2N(c2ccc(-c3ccc4ccccc4c3)cc2)c2ccc(-c3ccc4c(c3)oc3ccccc34)cc2)cc1 ; c1ccc(-c2ccc(-c3ccc4c(sc5ccccc54)c3N(c3ccc(-c4ccc5ccccc5c4)cc3)c3ccc(-c4ccc5c(c4)oc4ccccc45)cc3)cc2)cc1 ; c1ccc(-c2ccc3c(sc4ccccc43)c2N(c2ccc(-c3ccc4c(c3)oc3ccccc34)cc2)c2cc3ccccc3c3ccccc23)cc1]

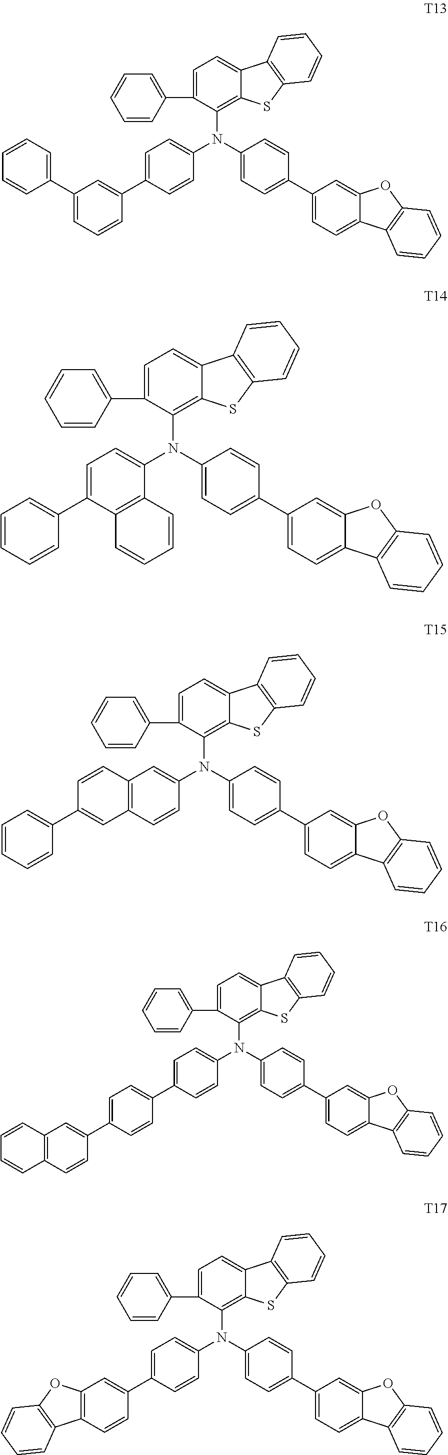 [SMILES: c1ccc(-c2cccc(-c3ccc(N(c4ccc(-c5ccc6c(c5)oc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3ccc4c(c3)oc3ccccc34)cc2)c2ccc(-c3ccccc3)c3ccccc23)cc1 ; c1ccc(-c2ccc3cc(N(c4ccc(-c5ccc6c(c5)oc5ccccc56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3ccc4c(c3)oc3ccccc34)cc2)cc1 ; c1ccc(-c2ccc3c(sc4ccccc43)c2N(c2ccc(-c3ccc4c(c3)oc3ccccc34)cc2)c2ccc(-c3ccc4c(c3)oc3ccccc34)cc2)cc1]

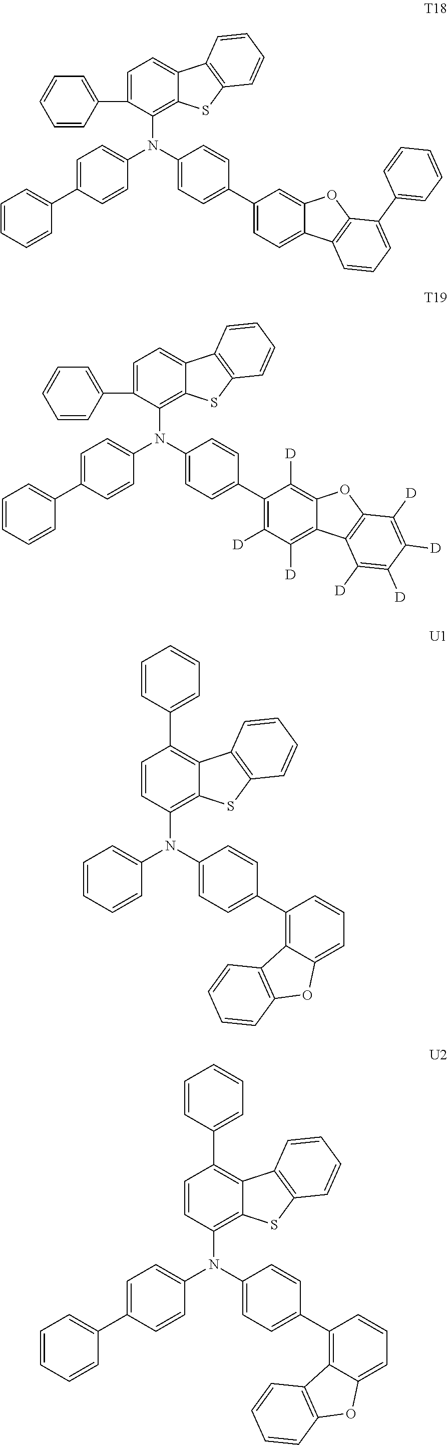 [SMILES: c1ccc(-c2ccc(N(c3ccc(-c4ccc5c(c4)oc4c(-c6ccccc6)cccc45)cc3)c3c(-c4ccccc4)ccc4c3sc3ccccc34)cc2)cc1 ; [2H]c1c([2H])c([2H])c2c(oc3c([2H])c(-c4ccc(N(c5ccc(-c6ccccc6)cc5)c5c(-c6ccccc6)ccc6c5sc5ccccc56)cc4)c([2H])c([2H])c32)c1[2H] ; c1ccc(-c2ccc(N(c3ccccc3)c3ccc(-c4cccc5oc6ccccc6c45)cc3)c3sc4ccccc4c23)cc1 ; c1ccc(-c2ccc(N(c3ccc(-c4cccc5oc6ccccc6c45)cc3)c3ccc(-c4ccccc4)c4c3sc3ccccc34)cc2)cc1]

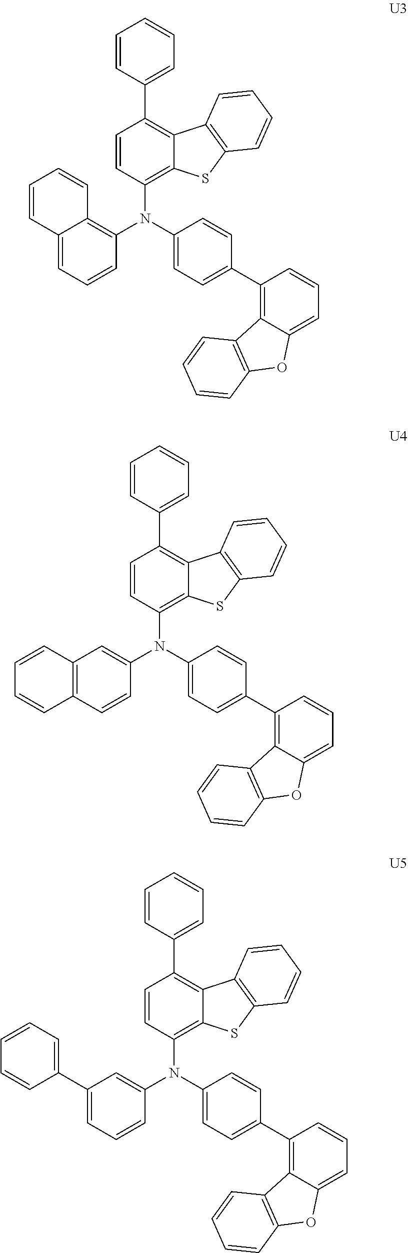 [SMILES: c1ccc(-c2ccc(N(c3ccc(-c4cccc5oc6ccccc6c45)cc3)c3cccc4ccccc34)c3sc4ccccc4c23)cc1 ; c1ccc(-c2ccc(N(c3ccc(-c4cccc5oc6ccccc6c45)cc3)c3ccc4ccccc4c3)c3sc4ccccc4c23)cc1 ; c1ccc(-c2cccc(N(c3ccc(-c4cccc5oc6ccccc6c45)cc3)c3ccc(-c4ccccc4)c4c3sc3ccccc34)c2)cc1]

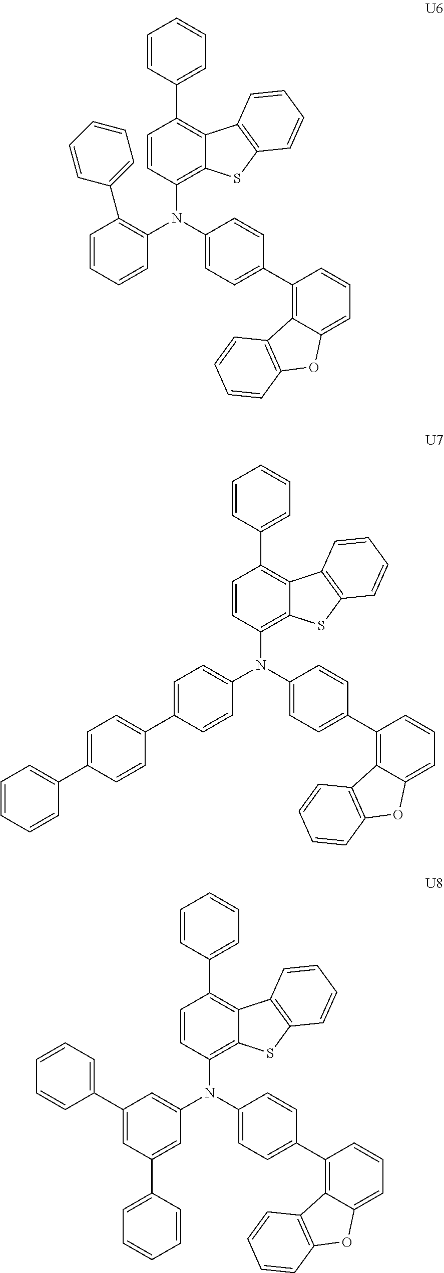 [SMILES: c1ccc(-c2ccccc2N(c2ccc(-c3cccc4oc5ccccc5c34)cc2)c2ccc(-c3ccccc3)c3c2sc2ccccc23)cc1 ; c1ccc(-c2ccc(-c3ccc(N(c4ccc(-c5cccc6oc7ccccc7c56)cc4)c4ccc(-c5ccccc5)c5c4sc4ccccc45)cc3)cc2)cc1 ; c1ccc(-c2cc(-c3ccccc3)cc(N(c3ccc(-c4cccc5oc6ccccc6c45)cc3)c3ccc(-c4ccccc4)c4c3sc3ccccc34)c2)cc1]

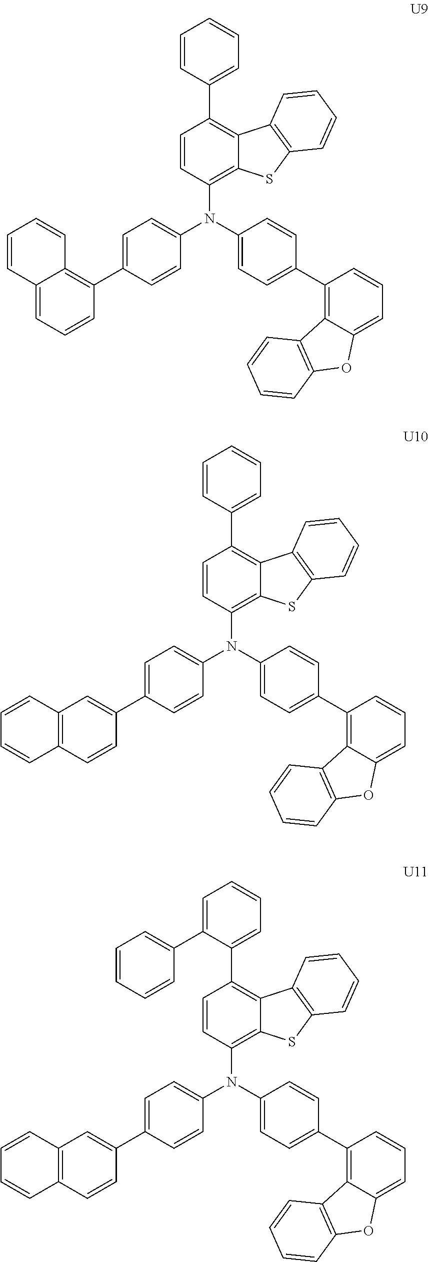 [SMILES: c1ccc(-c2ccc(N(c3ccc(-c4cccc5ccccc45)cc3)c3ccc(-c4cccc5oc6ccccc6c45)cc3)c3sc4ccccc4c23)cc1 ; c1ccc(-c2ccc(N(c3ccc(-c4ccc5ccccc5c4)cc3)c3ccc(-c4cccc5oc6ccccc6c45)cc3)c3sc4ccccc4c23)cc1 ; c1ccc(-c2ccccc2-c2ccc(N(c3ccc(-c4ccc5ccccc5c4)cc3)c3ccc(-c4cccc5oc6ccccc6c45)cc3)c3sc4ccccc4c23)cc1]

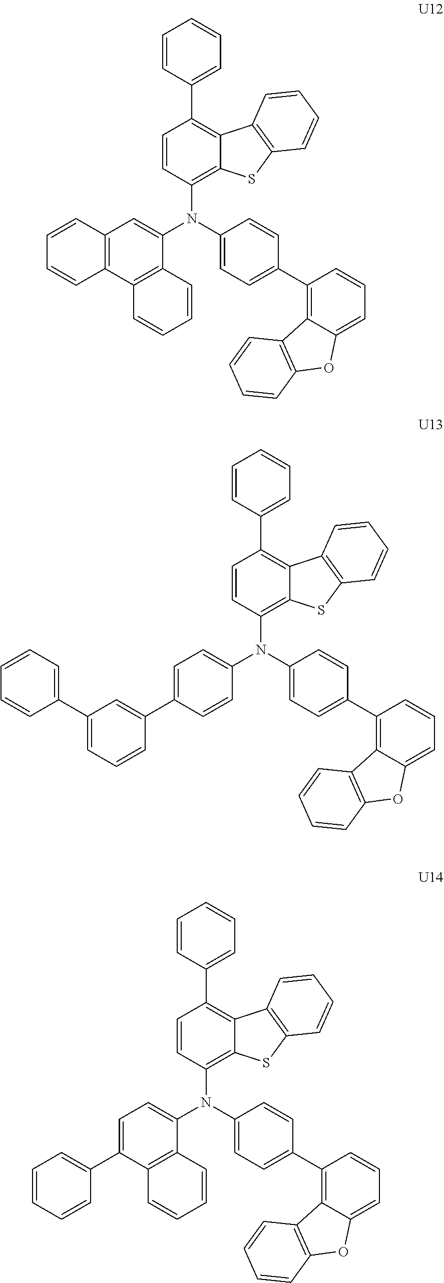 [SMILES: c1ccc(-c2ccc(N(c3ccc(-c4cccc5oc6ccccc6c45)cc3)c3cc4ccccc4c4ccccc34)c3sc4ccccc4c23)cc1 ; c1ccc(-c2cccc(-c3ccc(N(c4ccc(-c5cccc6oc7ccccc7c56)cc4)c4ccc(-c5ccccc5)c5c4sc4ccccc45)cc3)c2)cc1 ; c1ccc(-c2ccc(N(c3ccc(-c4cccc5oc6ccccc6c45)cc3)c3ccc(-c4ccccc4)c4c3sc3ccccc34)c3ccccc23)cc1]

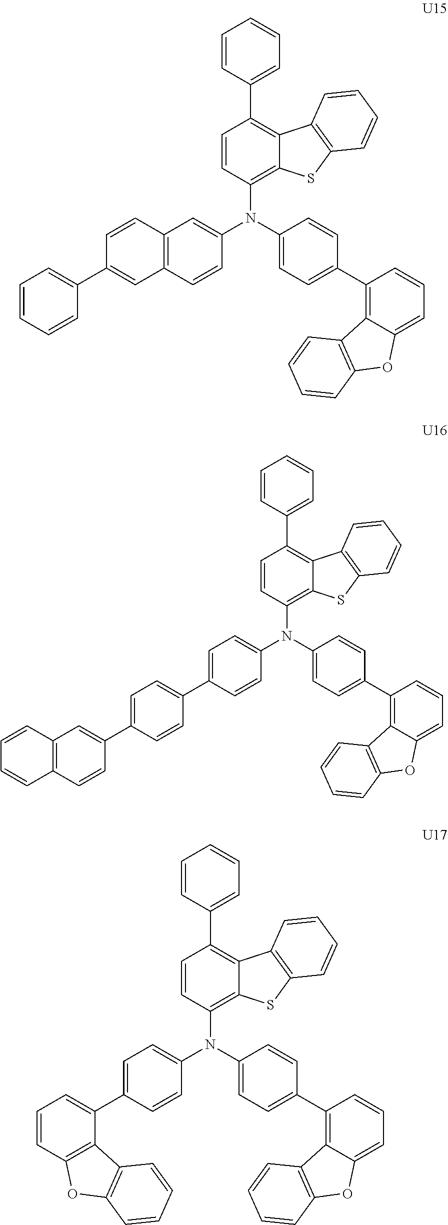 [SMILES: c1ccc(-c2ccc3cc(N(c4ccc(-c5cccc6oc7ccccc7c56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4cccc5oc6ccccc6c45)cc3)c3sc4ccccc4c23)cc1 ; c1ccc(-c2ccc(N(c3ccc(-c4cccc5oc6ccccc6c45)cc3)c3ccc(-c4cccc5oc6ccccc6c45)cc3)c3sc4ccccc4c23)cc1]

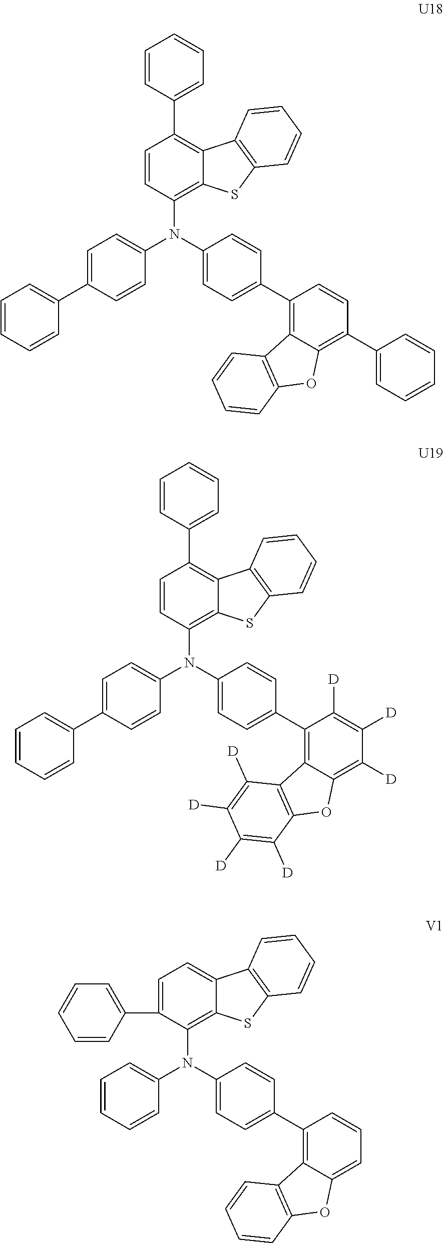 [SMILES: c1ccc(-c2ccc(N(c3ccc(-c4ccc(-c5ccccc5)c5oc6ccccc6c45)cc3)c3ccc(-c4ccccc4)c4c3sc3ccccc34)cc2)cc1 ; [2H]c1c([2H])c([2H])c2c(oc3c([2H])c([2H])c([2H])c(-c4ccc(N(c5ccc(-c6ccccc6)cc5)c5ccc(-c6ccccc6)c6c5sc5ccccc56)cc4)c32)c1[2H] ; c1ccc(-c2ccc3c(sc4ccccc43)c2N(c2ccccc2)c2ccc(-c3cccc4oc5ccccc5c34)cc2)cc1]

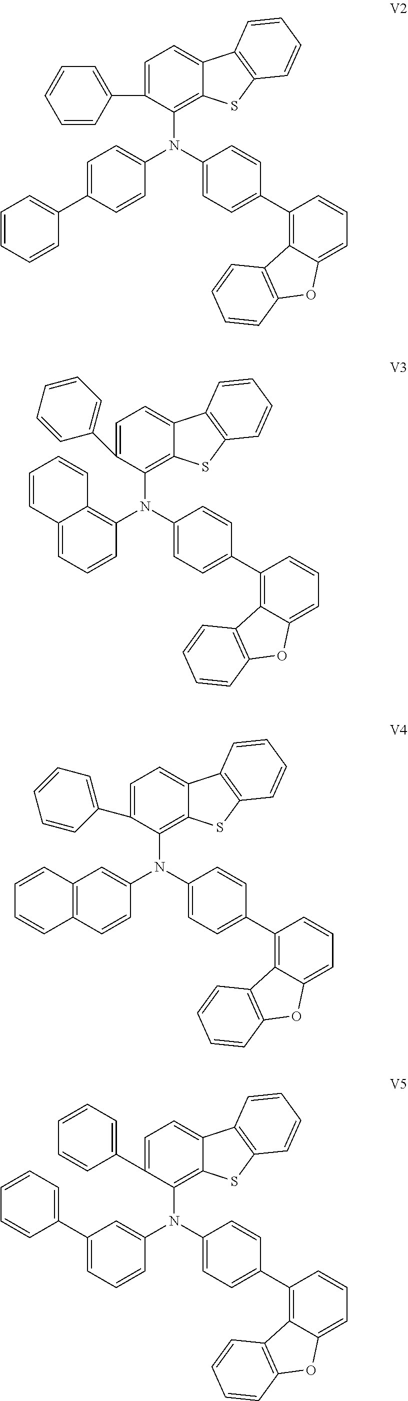 [SMILES: c1ccc(-c2ccc(N(c3ccc(-c4cccc5oc6ccccc6c45)cc3)c3c(-c4ccccc4)ccc4c3sc3ccccc34)cc2)cc1 ; c1ccc(-c2ccc3c(sc4ccccc43)c2N(c2ccc(-c3cccc4oc5ccccc5c34)cc2)c2cccc3ccccc23)cc1 ; c1ccc(-c2ccc3c(sc4ccccc43)c2N(c2ccc(-c3cccc4oc5ccccc5c34)cc2)c2ccc3ccccc3c2)cc1 ; c1ccc(-c2cccc(N(c3ccc(-c4cccc5oc6ccccc6c45)cc3)c3c(-c4ccccc4)ccc4c3sc3ccccc34)c2)cc1]

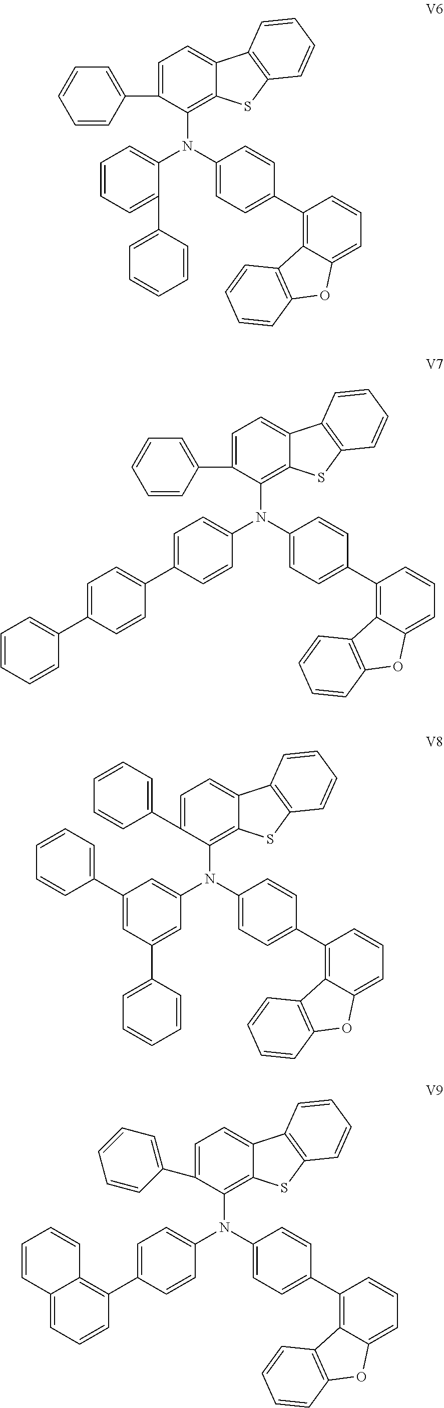

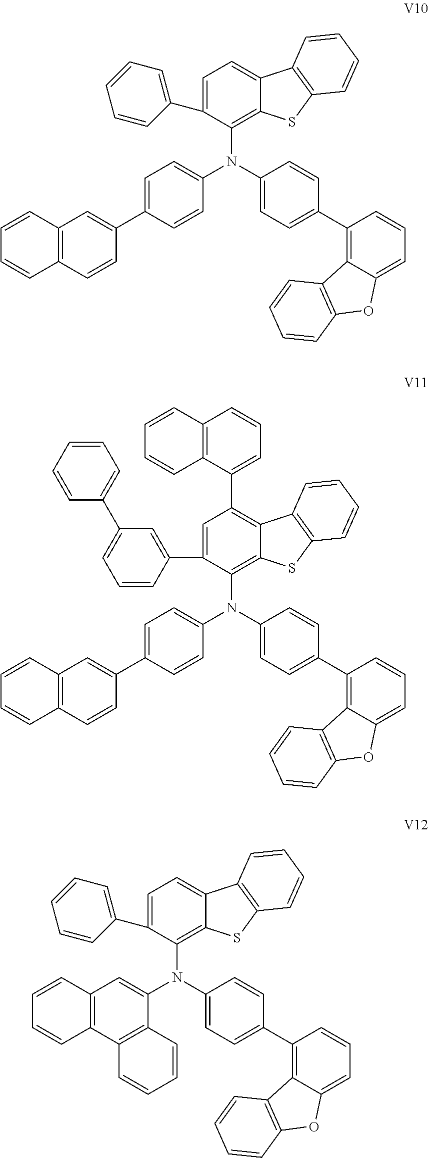 [SMILES: c1ccc(-c2ccc3c(sc4ccccc43)c2N(c2ccc(-c3ccc4ccccc4c3)cc2)c2ccc(-c3cccc4oc5ccccc5c34)cc2)cc1 ; c1ccc(-c2cccc(-c3cc(-c4cccc5ccccc45)c4c(sc5ccccc54)c3N(c3ccc(-c4ccc5ccccc5c4)cc3)c3ccc(-c4cccc5oc6ccccc6c45)cc3)c2)cc1 ; c1ccc(-c2ccc3c(sc4ccccc43)c2N(c2ccc(-c3cccc4oc5ccccc5c34)cc2)c2cc3ccccc3c3ccccc23)cc1]

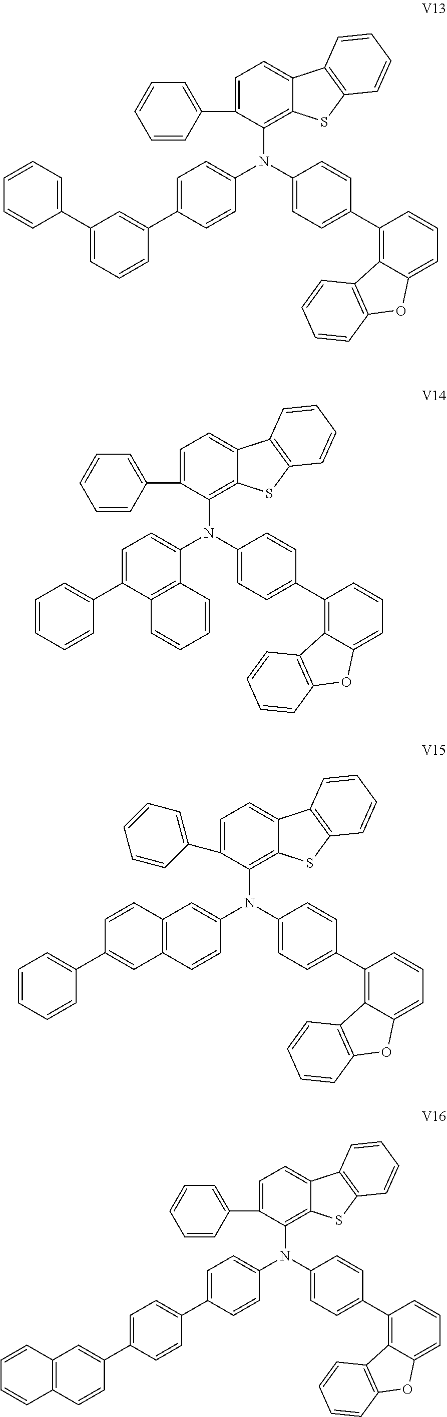 [SMILES: c1ccc(-c2cccc(-c3ccc(N(c4ccc(-c5cccc6oc7ccccc7c56)cc4)c4c(-c5ccccc5)ccc5c4sc4ccccc45)cc3)c2)cc1 ; c1ccc(-c2ccc3c(sc4ccccc43)c2N(c2ccc(-c3cccc4oc5ccccc5c34)cc2)c2ccc(-c3ccccc3)c3ccccc23)cc1 ; c1ccc(-c2ccc3cc(N(c4ccc(-c5cccc6oc7ccccc7c56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3cccc4oc5ccccc5c34)cc2)cc1]

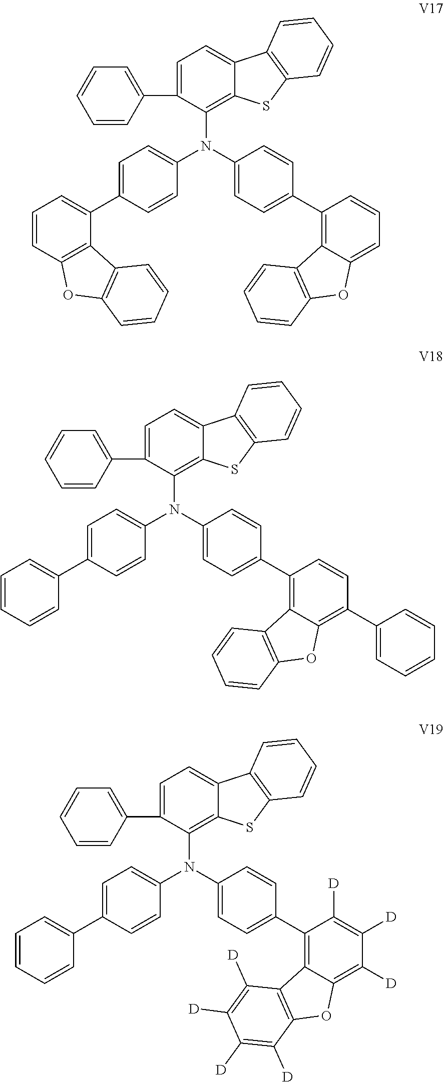 [SMILES: c1ccc(-c2ccc3c(sc4ccccc43)c2N(c2ccc(-c3cccc4oc5ccccc5c34)cc2)c2ccc(-c3cccc4oc5ccccc5c34)cc2)cc1 ; c1ccc(-c2ccc(N(c3ccc(-c4ccc(-c5ccccc5)c5oc6ccccc6c45)cc3)c3c(-c4ccccc4)ccc4c3sc3ccccc34)cc2)cc1 ; [2H]c1c([2H])c([2H])c2c(oc3c([2H])c([2H])c([2H])c(-c4ccc(N(c5ccc(-c6ccccc6)cc5)c5c(-c6ccccc6)ccc6c5sc5ccccc56)cc4)c32)c1[2H]]

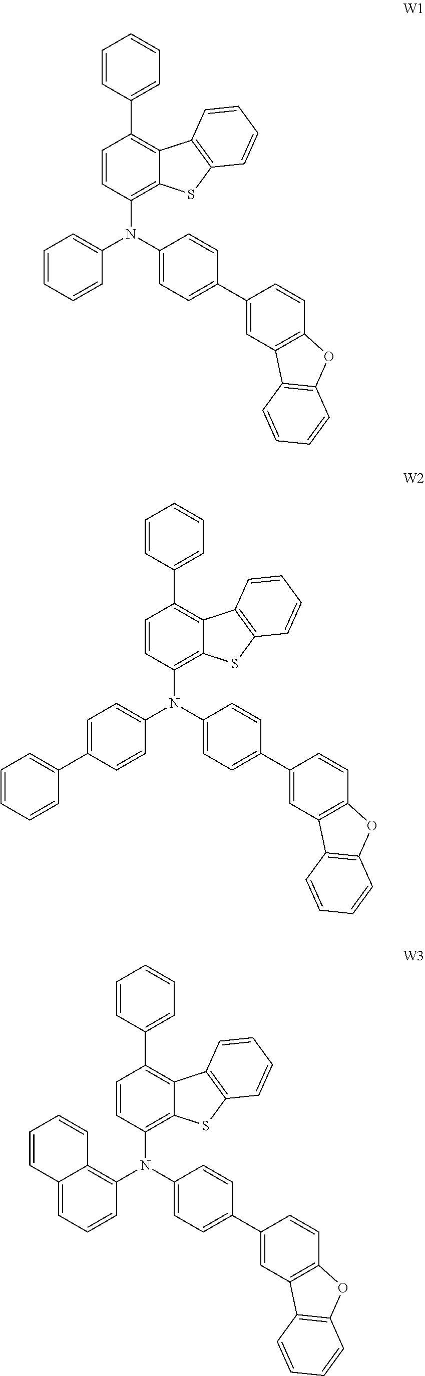 [SMILES: c1ccc(-c2ccc(N(c3ccccc3)c3ccc(-c4ccc5oc6ccccc6c5c4)cc3)c3sc4ccccc4c23)cc1 ; c1ccc(-c2ccc(N(c3ccc(-c4ccc5oc6ccccc6c5c4)cc3)c3ccc(-c4ccccc4)c4c3sc3ccccc34)cc2)cc1 ; c1ccc(-c2ccc(N(c3ccc(-c4ccc5oc6ccccc6c5c4)cc3)c3cccc4ccccc34)c3sc4ccccc4c23)cc1]

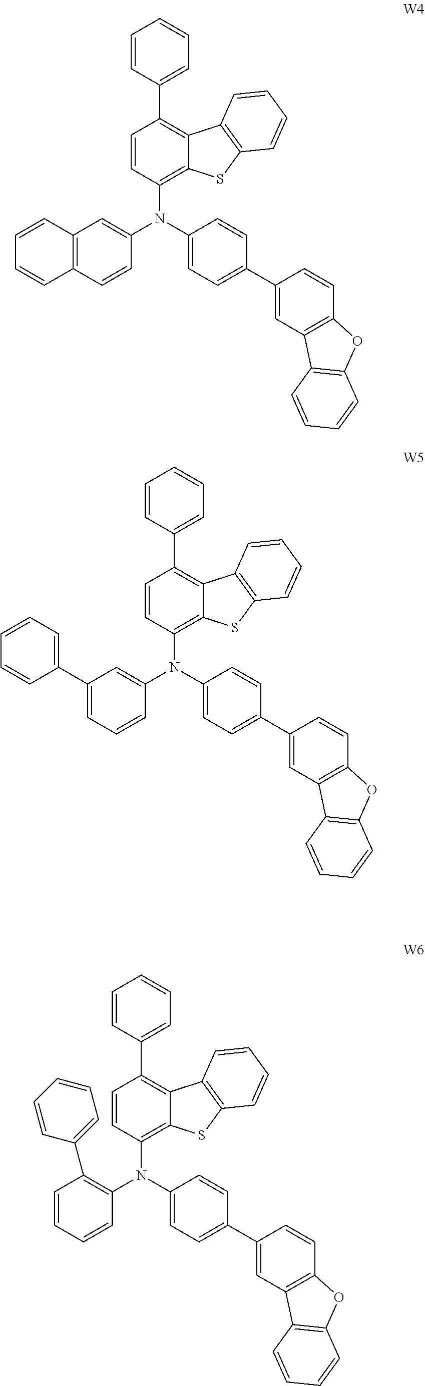 [SMILES: c1ccc(-c2ccc(N(c3ccc(-c4ccc5oc6ccccc6c5c4)cc3)c3ccc4ccccc4c3)c3sc4ccccc4c23)cc1 ; c1ccc(-c2cccc(N(c3ccc(-c4ccc5oc6ccccc6c5c4)cc3)c3ccc(-c4ccccc4)c4c3sc3ccccc34)c2)cc1 ; c1ccc(-c2ccccc2N(c2ccc(-c3ccc4oc5ccccc5c4c3)cc2)c2ccc(-c3ccccc3)c3c2sc2ccccc23)cc1]

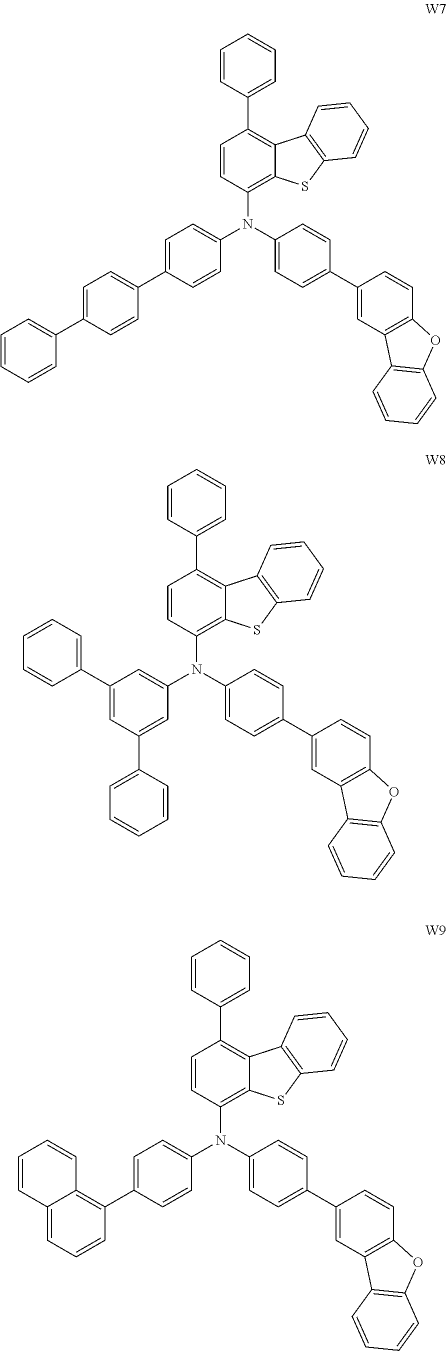 [SMILES: c1ccc(-c2ccc(-c3ccc(N(c4ccc(-c5ccc6oc7ccccc7c6c5)cc4)c4ccc(-c5ccccc5)c5c4sc4ccccc45)cc3)cc2)cc1 ; c1ccc(-c2cc(-c3ccccc3)cc(N(c3ccc(-c4ccc5oc6ccccc6c5c4)cc3)c3ccc(-c4ccccc4)c4c3sc3ccccc34)c2)cc1 ; c1ccc(-c2ccc(N(c3ccc(-c4ccc5oc6ccccc6c5c4)cc3)c3ccc(-c4cccc5ccccc45)cc3)c3sc4ccccc4c23)cc1]

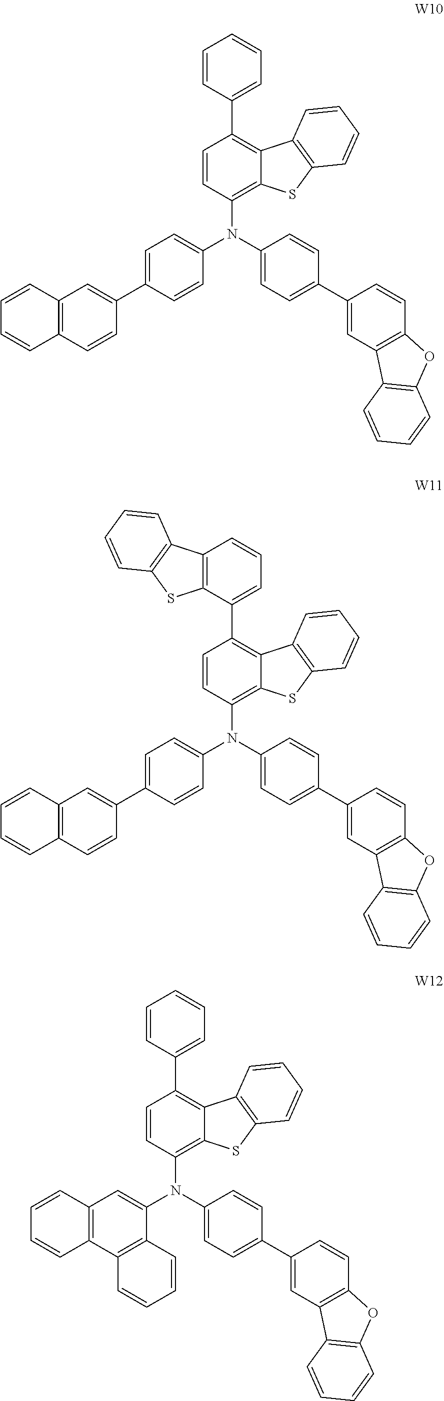 [SMILES: c1ccc(-c2ccc(N(c3ccc(-c4ccc5ccccc5c4)cc3)c3ccc(-c4ccc5oc6ccccc6c5c4)cc3)c3sc4ccccc4c23)cc1 ; c1ccc2cc(-c3ccc(N(c4ccc(-c5ccc6oc7ccccc7c6c5)cc4)c4ccc(-c5cccc6c5sc5ccccc56)c5c4sc4ccccc45)cc3)ccc2c1 ; c1ccc(-c2ccc(N(c3ccc(-c4ccc5oc6ccccc6c5c4)cc3)c3cc4ccccc4c4ccccc34)c3sc4ccccc4c23)cc1]

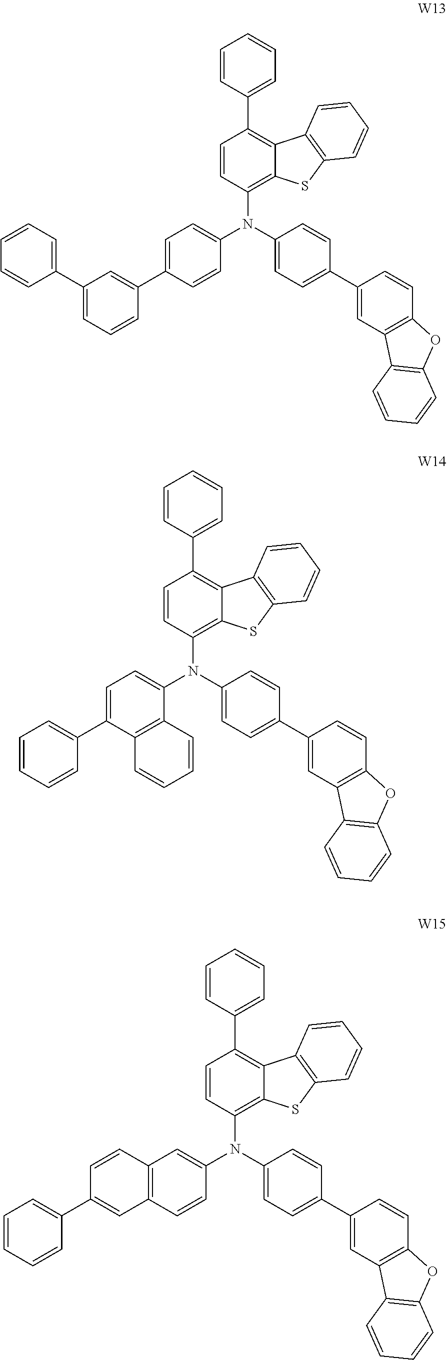 [SMILES: c1ccc(-c2cccc(-c3ccc(N(c4ccc(-c5ccc6oc7ccccc7c6c5)cc4)c4ccc(-c5ccccc5)c5c4sc4ccccc45)cc3)c2)cc1 ; c1ccc(-c2ccc(N(c3ccc(-c4ccc5oc6ccccc6c5c4)cc3)c3ccc(-c4ccccc4)c4c3sc3ccccc34)c3ccccc23)cc1 ; c1ccc(-c2ccc3cc(N(c4ccc(-c5ccc6oc7ccccc7c6c5)cc4)c4ccc(-c5ccccc5)c5c4sc4ccccc45)ccc3c2)cc1]

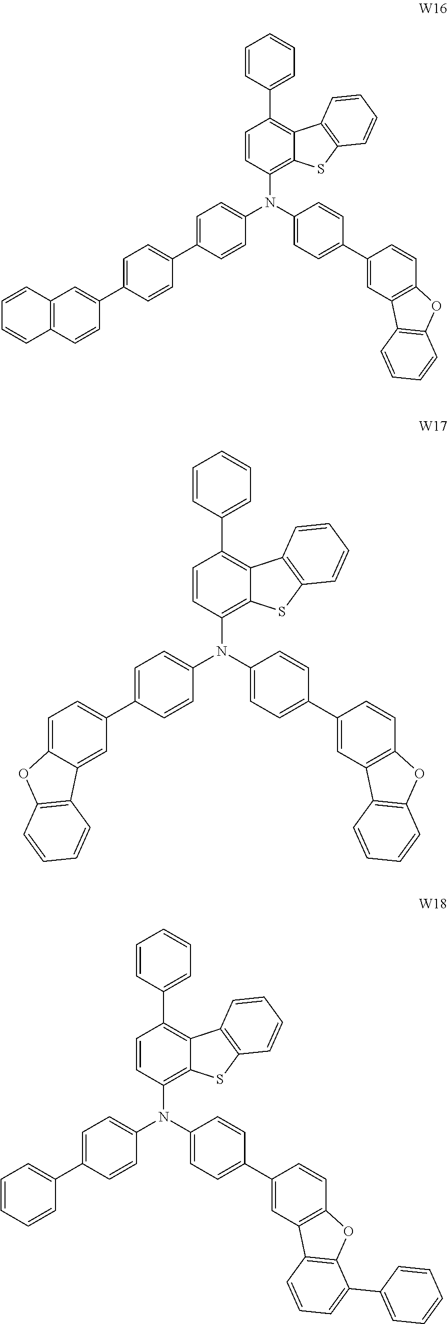 [SMILES: c1ccc(-c2ccc(N(c3ccc(-c4ccc(-c5ccc6ccccc6c5)cc4)cc3)c3ccc(-c4ccc5oc6ccccc6c5c4)cc3)c3sc4ccccc4c23)cc1 ; c1ccc(-c2ccc(N(c3ccc(-c4ccc5oc6ccccc6c5c4)cc3)c3ccc(-c4ccc5oc6ccccc6c5c4)cc3)c3sc4ccccc4c23)cc1 ; c1ccc(-c2ccc(N(c3ccc(-c4ccc5oc6c(-c7ccccc7)cccc6c5c4)cc3)c3ccc(-c4ccccc4)c4c3sc3ccccc34)cc2)cc1]

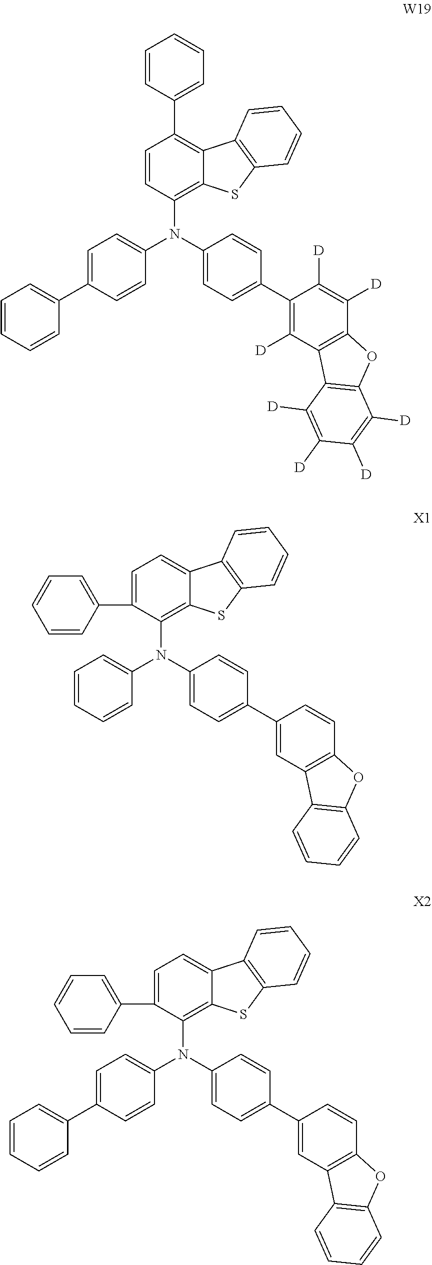 [SMILES: [2H]c1c([2H])c([2H])c2c(oc3c([2H])c([2H])c(-c4ccc(N(c5ccc(-c6ccccc6)cc5)c5ccc(-c6ccccc6)c6c5sc5ccccc56)cc4)c([2H])c32)c1[2H] ; c1ccc(-c2ccc3c(sc4ccccc43)c2N(c2ccccc2)c2ccc(-c3ccc4oc5ccccc5c4c3)cc2)cc1 ; c1ccc(-c2ccc(N(c3ccc(-c4ccc5oc6ccccc6c5c4)cc3)c3c(-c4ccccc4)ccc4c3sc3ccccc34)cc2)cc1]

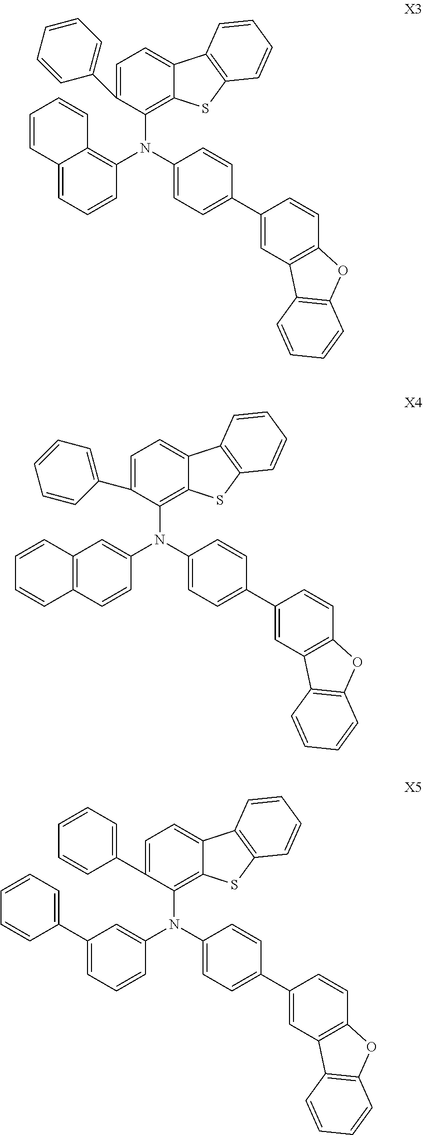 [SMILES: c1ccc(-c2ccc3c(sc4ccccc43)c2N(c2ccc(-c3ccc4oc5ccccc5c4c3)cc2)c2cccc3ccccc23)cc1 ; c1ccc(-c2ccc3c(sc4ccccc43)c2N(c2ccc(-c3ccc4oc5ccccc5c4c3)cc2)c2ccc3ccccc3c2)cc1 ; c1ccc(-c2cccc(N(c3ccc(-c4ccc5oc6ccccc6c5c4)cc3)c3c(-c4ccccc4)ccc4c3sc3ccccc34)c2)cc1]

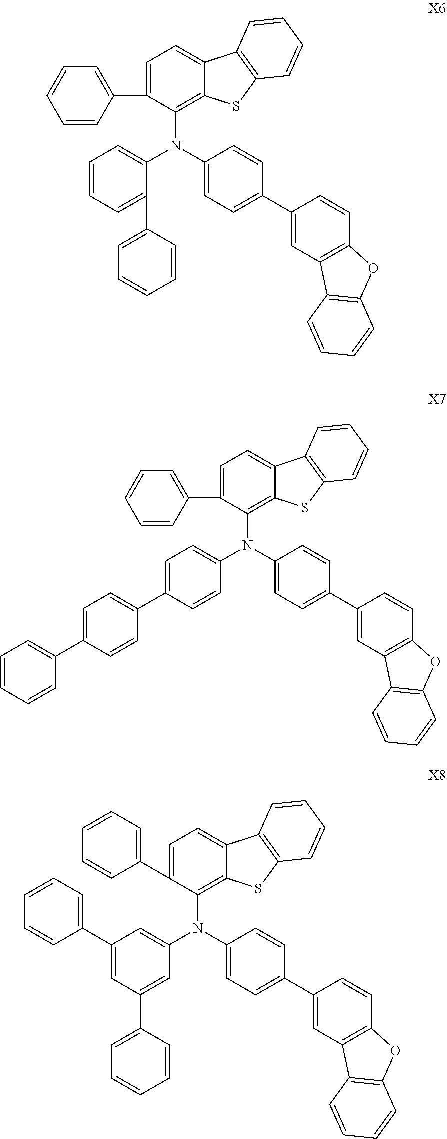 [SMILES: c1ccc(-c2ccccc2N(c2ccc(-c3ccc4oc5ccccc5c4c3)cc2)c2c(-c3ccccc3)ccc3c2sc2ccccc23)cc1 ; c1ccc(-c2ccc(-c3ccc(N(c4ccc(-c5ccc6oc7ccccc7c6c5)cc4)c4c(-c5ccccc5)ccc5c4sc4ccccc45)cc3)cc2)cc1 ; c1ccc(-c2cc(-c3ccccc3)cc(N(c3ccc(-c4ccc5oc6ccccc6c5c4)cc3)c3c(-c4ccccc4)ccc4c3sc3ccccc34)c2)cc1]

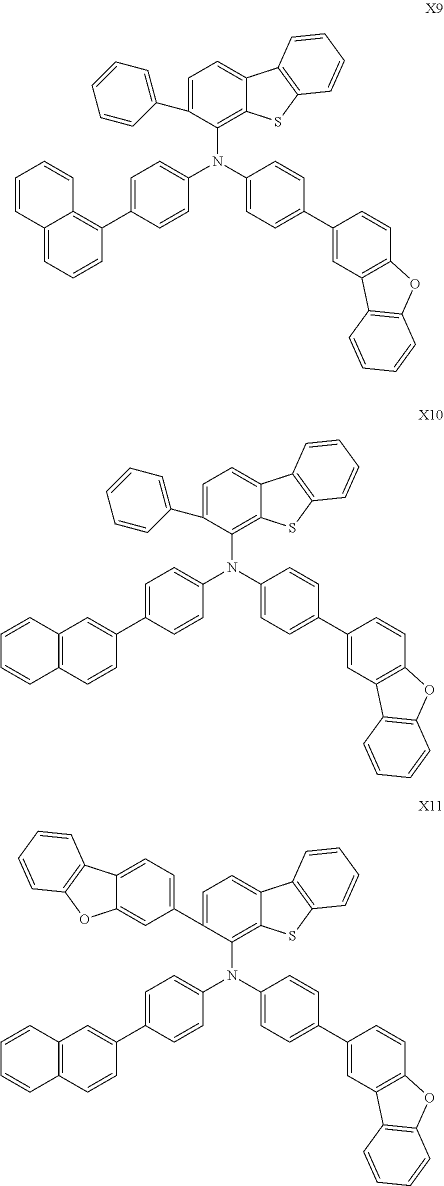 [SMILES: c1ccc(-c2ccc3c(sc4ccccc43)c2N(c2ccc(-c3ccc4oc5ccccc5c4c3)cc2)c2ccc(-c3cccc4ccccc34)cc2)cc1 ; c1ccc(-c2ccc3c(sc4ccccc43)c2N(c2ccc(-c3ccc4ccccc4c3)cc2)c2ccc(-c3ccc4oc5ccccc5c4c3)cc2)cc1 ; c1ccc2cc(-c3ccc(N(c4ccc(-c5ccc6oc7ccccc7c6c5)cc4)c4c(-c5ccc6c(c5)oc5ccccc56)ccc5c4sc4ccccc45)cc3)ccc2c1]

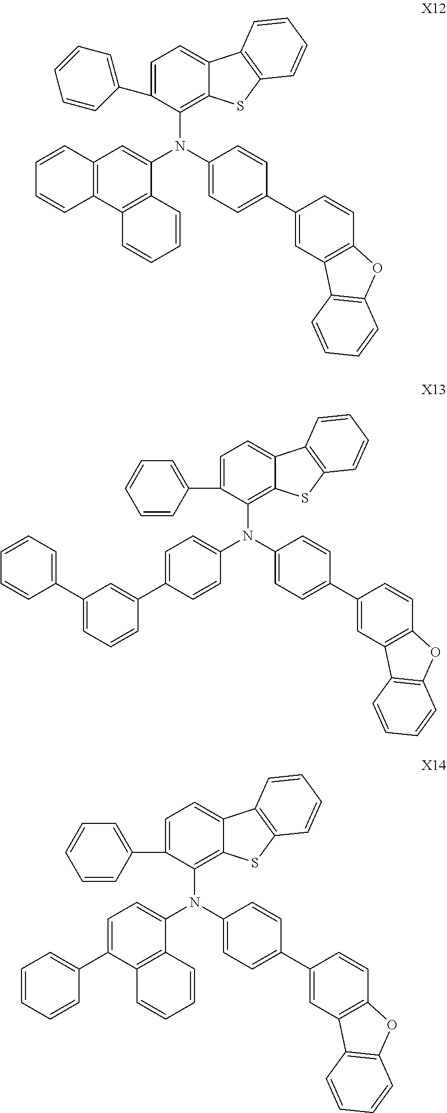 [SMILES: c1ccc(-c2ccc3c(sc4ccccc43)c2N(c2ccc(-c3ccc4oc5ccccc5c4c3)cc2)c2cc3ccccc3c3ccccc23)cc1 ; c1ccc(-c2cccc(-c3ccc(N(c4ccc(-c5ccc6oc7ccccc7c6c5)cc4)c4c(-c5ccccc5)ccc5c4sc4ccccc45)cc3)c2)cc1 ; c1ccc(-c2ccc3c(sc4ccccc43)c2N(c2ccc(-c3ccc4oc5ccccc5c4c3)cc2)c2ccc(-c3ccccc3)c3ccccc23)cc1]

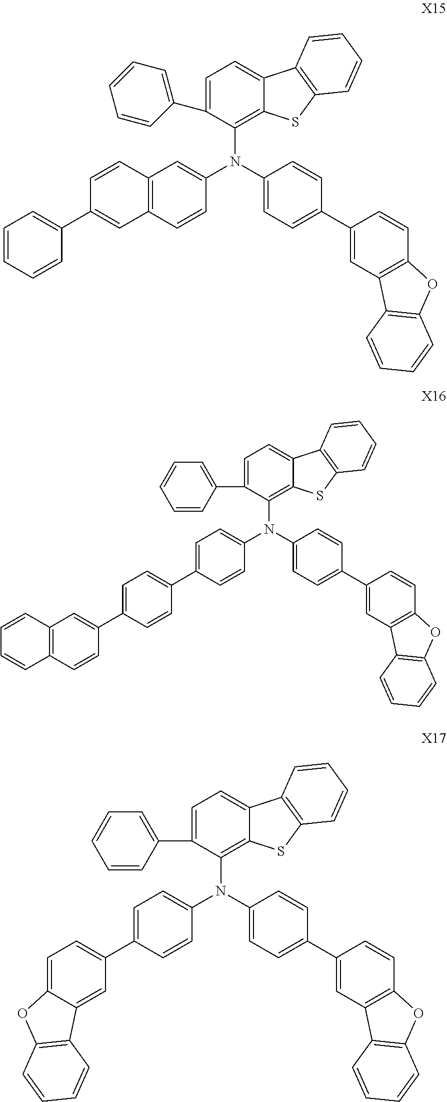 [SMILES: c1ccc(-c2ccc3cc(N(c4ccc(-c5ccc6oc7ccccc7c6c5)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3ccc4oc5ccccc5c4c3)cc2)cc1 ; c1ccc(-c2ccc3c(sc4ccccc43)c2N(c2ccc(-c3ccc4oc5ccccc5c4c3)cc2)c2ccc(-c3ccc4oc5ccccc5c4c3)cc2)cc1]

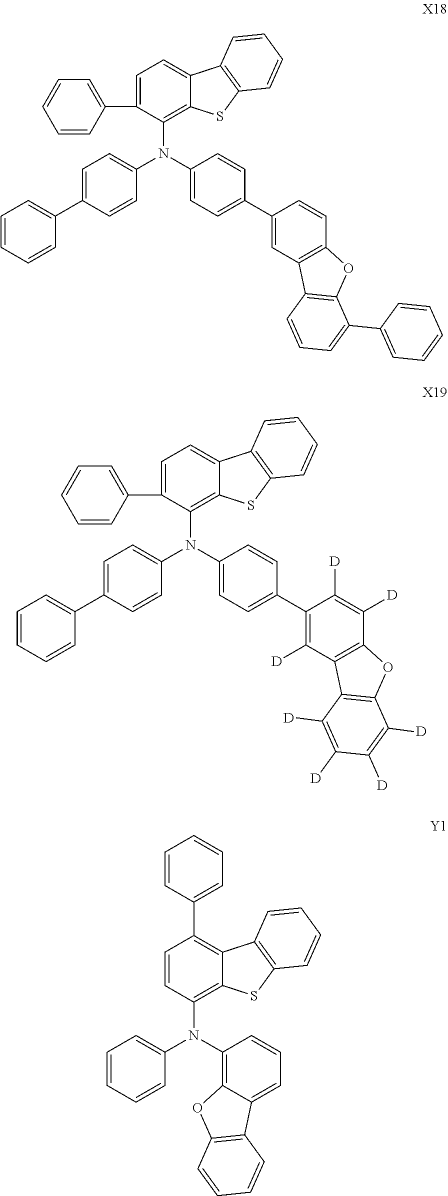 [SMILES: c1ccc(-c2ccc(N(c3ccc(-c4ccc5oc6c(-c7ccccc7)cccc6c5c4)cc3)c3c(-c4ccccc4)ccc4c3sc3ccccc34)cc2)cc1 ; [2H]c1c([2H])c([2H])c2c(oc3c([2H])c([2H])c(-c4ccc(N(c5ccc(-c6ccccc6)cc5)c5c(-c6ccccc6)ccc6c5sc5ccccc56)cc4)c([2H])c32)c1[2H] ; c1ccc(-c2ccc(N(c3ccccc3)c3cccc4c3oc3ccccc34)c3sc4ccccc4c23)cc1]

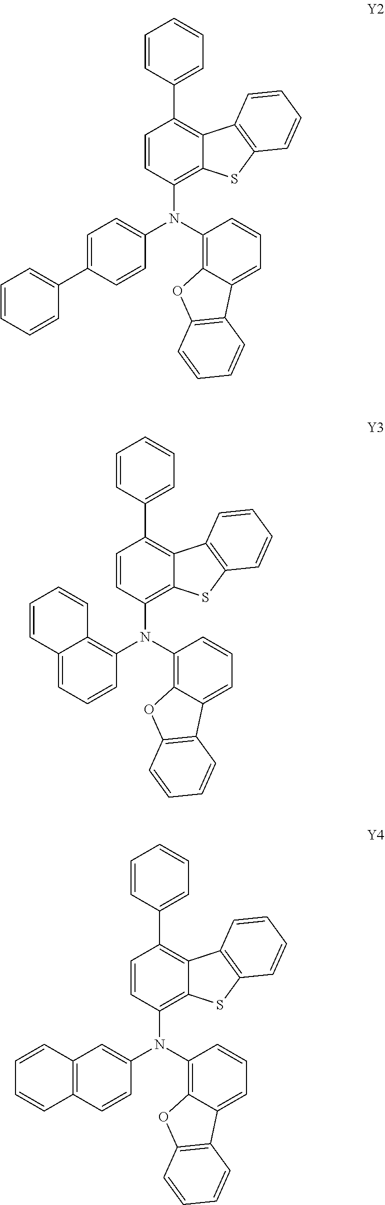 [SMILES: c1ccc(-c2ccc(N(c3cccc4c3oc3ccccc34)c3ccc(-c4ccccc4)c4c3sc3ccccc34)cc2)cc1 ; c1ccc(-c2ccc(N(c3cccc4ccccc34)c3cccc4c3oc3ccccc34)c3sc4ccccc4c23)cc1 ; c1ccc(-c2ccc(N(c3ccc4ccccc4c3)c3cccc4c3oc3ccccc34)c3sc4ccccc4c23)cc1]

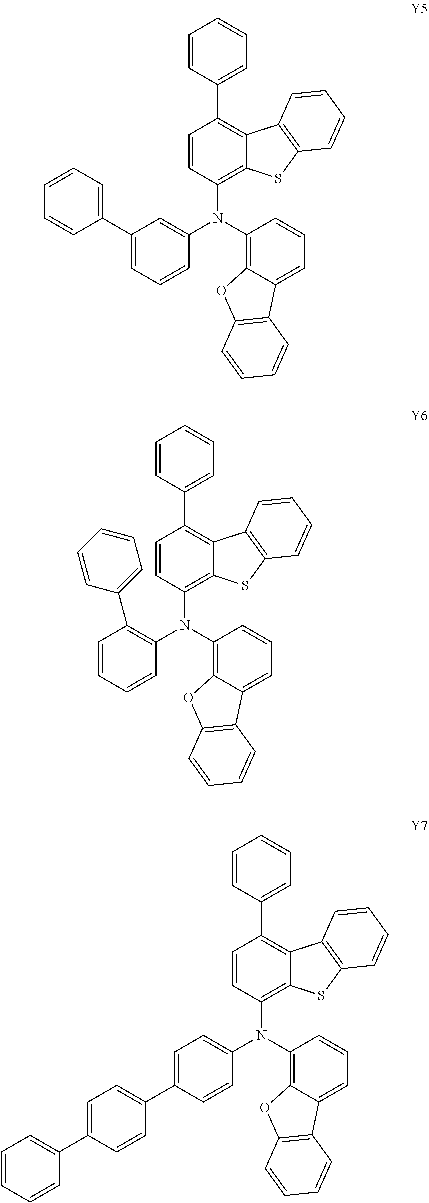 [SMILES: c1ccc(-c2cccc(N(c3cccc4c3oc3ccccc34)c3ccc(-c4ccccc4)c4c3sc3ccccc34)c2)cc1 ; c1ccc(-c2ccccc2N(c2cccc3c2oc2ccccc23)c2ccc(-c3ccccc3)c3c2sc2ccccc23)cc1 ; c1ccc(-c2ccc(-c3ccc(N(c4cccc5c4oc4ccccc45)c4ccc(-c5ccccc5)c5c4sc4ccccc45)cc3)cc2)cc1]

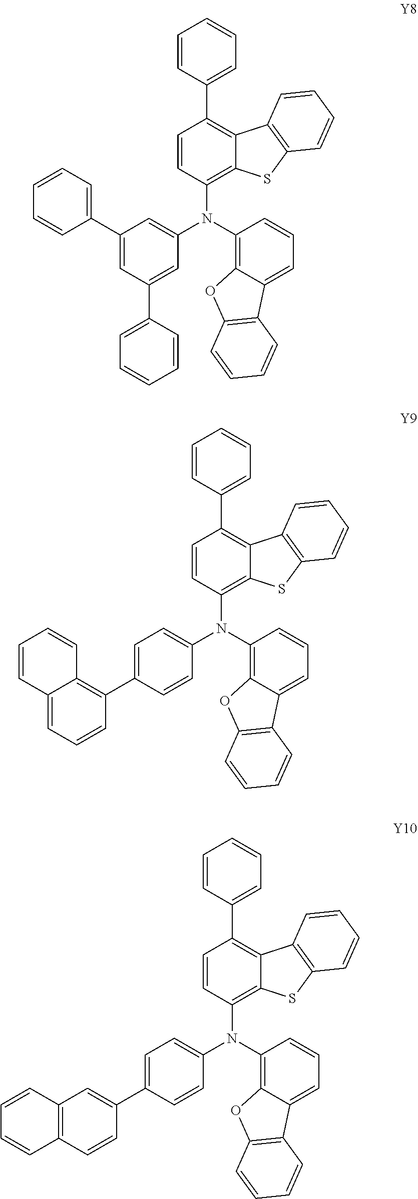 [SMILES: c1ccc(-c2cc(-c3ccccc3)cc(N(c3cccc4c3oc3ccccc34)c3ccc(-c4ccccc4)c4c3sc3ccccc34)c2)cc1 ; c1ccc(-c2ccc(N(c3ccc(-c4cccc5ccccc45)cc3)c3cccc4c3oc3ccccc34)c3sc4ccccc4c23)cc1 ; c1ccc(-c2ccc(N(c3ccc(-c4ccc5ccccc5c4)cc3)c3cccc4c3oc3ccccc34)c3sc4ccccc4c23)cc1]

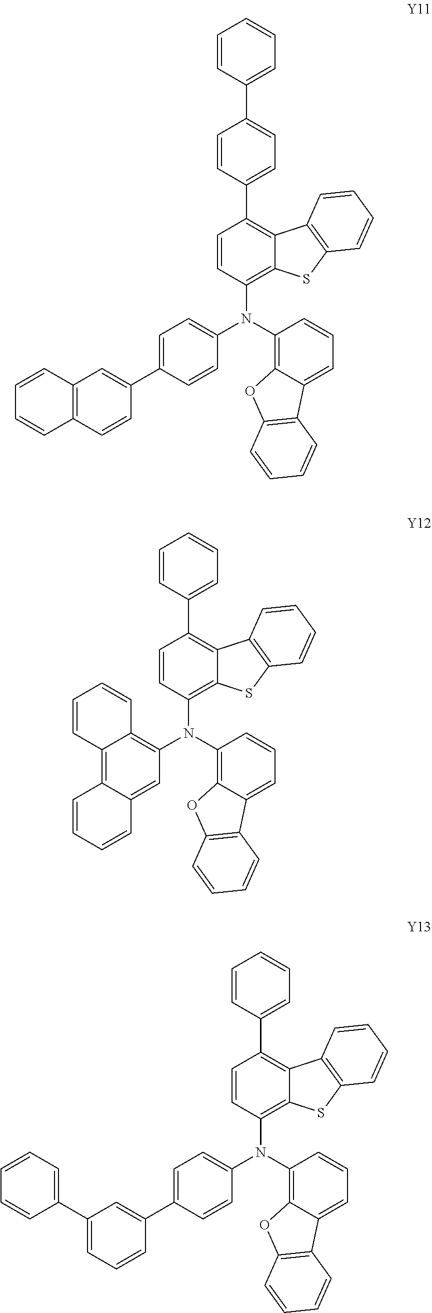 [SMILES: c1ccc(-c2ccc(-c3ccc(N(c4ccc(-c5ccc6ccccc6c5)cc4)c4cccc5c4oc4ccccc45)c4sc5ccccc5c34)cc2)cc1 ; c1ccc(-c2ccc(N(c3cc4ccccc4c4ccccc34)c3cccc4c3oc3ccccc34)c3sc4ccccc4c23)cc1 ; c1ccc(-c2cccc(-c3ccc(N(c4cccc5c4oc4ccccc45)c4ccc(-c5ccccc5)c5c4sc4ccccc45)cc3)c2)cc1]

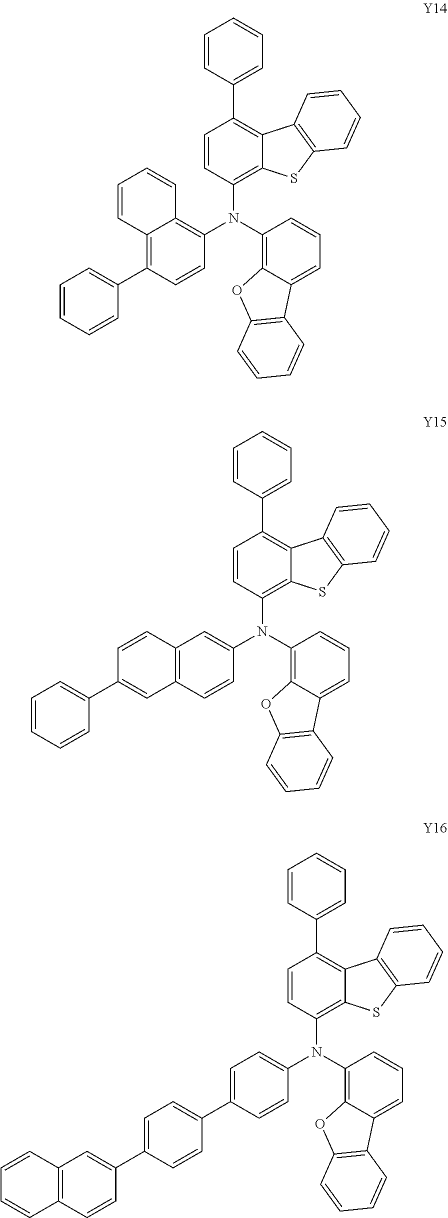 [SMILES: c1ccc(-c2ccc(N(c3cccc4c3oc3ccccc34)c3ccc(-c4ccccc4)c4c3sc3ccccc34)c3ccccc23)cc1 ; c1ccc(-c2ccc3cc(N(c4cccc5c4oc4ccccc45)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3cccc4c3oc3ccccc34)c3sc4ccccc4c23)cc1]

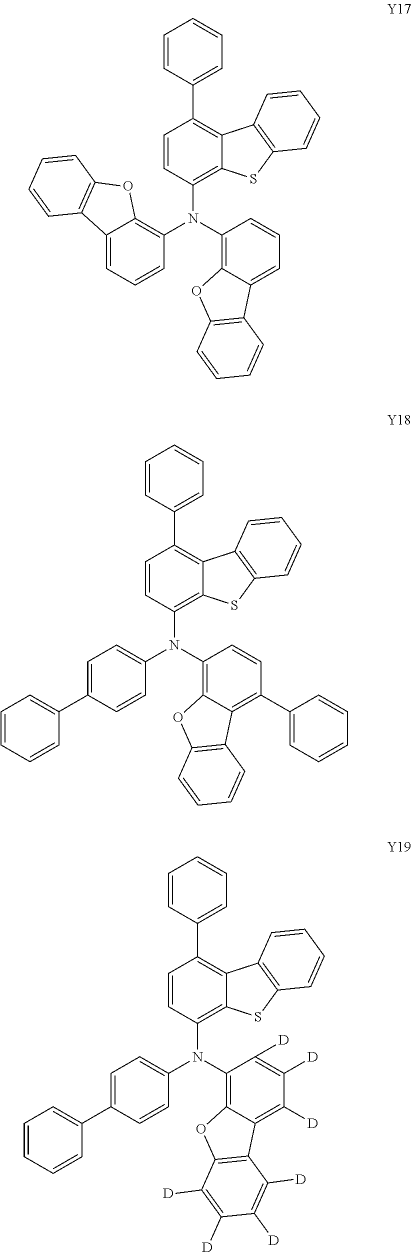 [SMILES: c1ccc(-c2ccc(N(c3cccc4c3oc3ccccc34)c3cccc4c3oc3ccccc34)c3sc4ccccc4c23)cc1 ; c1ccc(-c2ccc(N(c3ccc(-c4ccccc4)c4c3oc3ccccc34)c3ccc(-c4ccccc4)c4c3sc3ccccc34)cc2)cc1 ; [2H]c1c([2H])c([2H])c2c(oc3c(N(c4ccc(-c5ccccc5)cc4)c4ccc(-c5ccccc5)c5c4sc4ccccc45)c([2H])c([2H])c([2H])c32)c1[2H]]

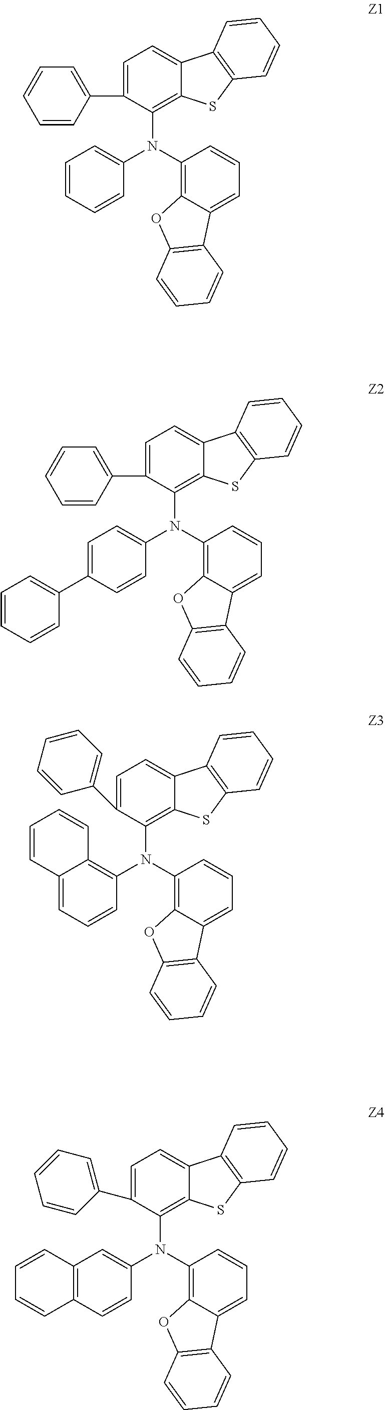 [SMILES: c1ccc(-c2ccc3c(sc4ccccc43)c2N(c2ccccc2)c2cccc3c2oc2ccccc23)cc1 ; c1ccc(-c2ccc(N(c3cccc4c3oc3ccccc34)c3c(-c4ccccc4)ccc4c3sc3ccccc34)cc2)cc1 ; c1ccc(-c2ccc3c(sc4ccccc43)c2N(c2cccc3ccccc23)c2cccc3c2oc2ccccc23)cc1 ; c1ccc(-c2ccc3c(sc4ccccc43)c2N(c2ccc3ccccc3c2)c2cccc3c2oc2ccccc23)cc1]

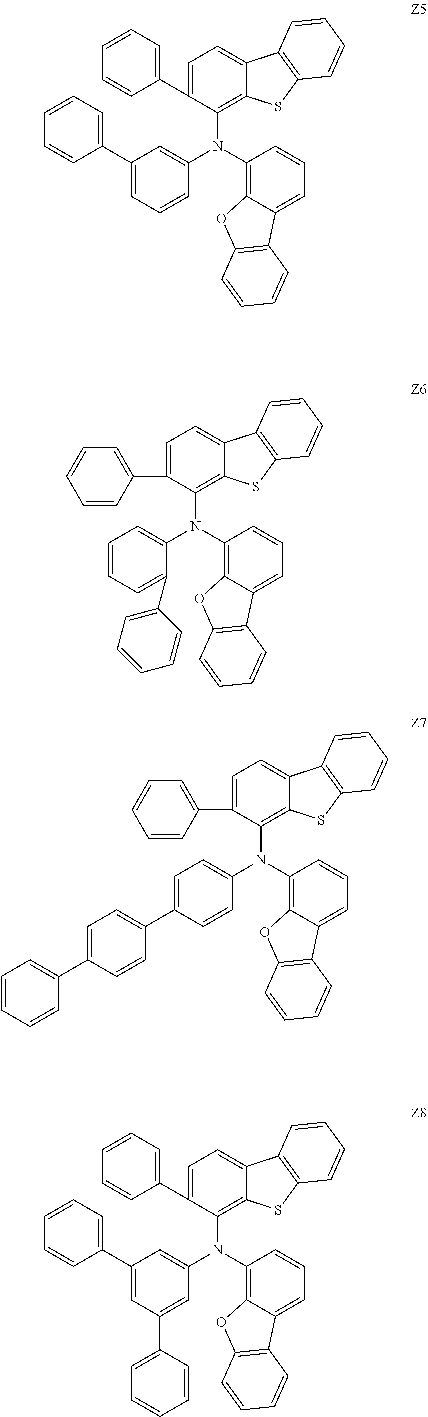 [SMILES: c1ccc(-c2cccc(N(c3cccc4c3oc3ccccc34)c3c(-c4ccccc4)ccc4c3sc3ccccc34)c2)cc1 ; c1ccc(-c2ccccc2N(c2cccc3c2oc2ccccc23)c2c(-c3ccccc3)ccc3c2sc2ccccc23)cc1 ; c1ccc(-c2ccc(-c3ccc(N(c4cccc5c4oc4ccccc45)c4c(-c5ccccc5)ccc5c4sc4ccccc45)cc3)cc2)cc1 ; c1ccc(-c2cc(-c3ccccc3)cc(N(c3cccc4c3oc3ccccc34)c3c(-c4ccccc4)ccc4c3sc3ccccc34)c2)cc1]

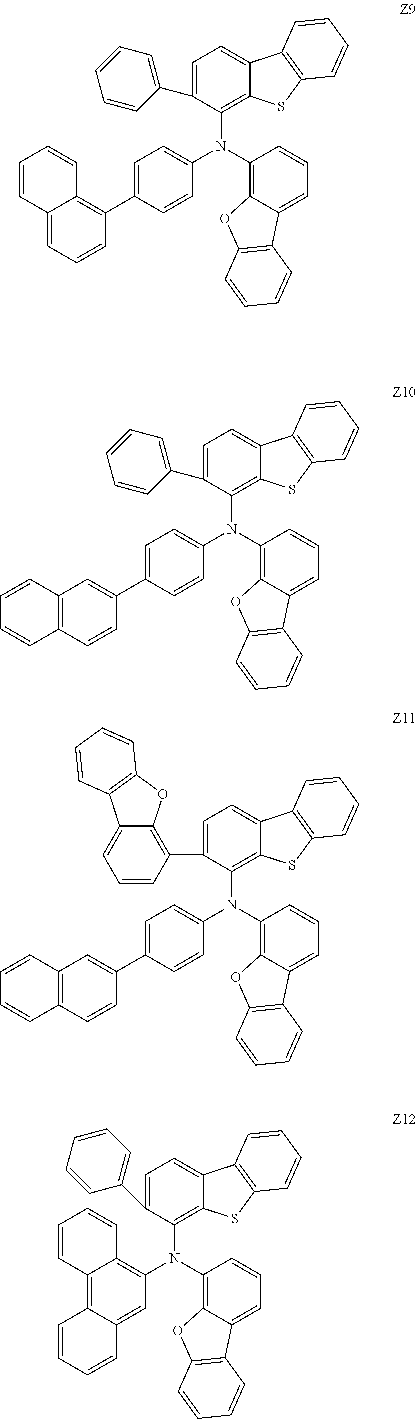 [SMILES: c1ccc(-c2ccc3c(sc4ccccc43)c2N(c2ccc(-c3cccc4ccccc34)cc2)c2cccc3c2oc2ccccc23)cc1 ; c1ccc(-c2ccc3c(sc4ccccc43)c2N(c2ccc(-c3ccc4ccccc4c3)cc2)c2cccc3c2oc2ccccc23)cc1 ; c1ccc2cc(-c3ccc(N(c4cccc5c4oc4ccccc45)c4c(-c5cccc6c5oc5ccccc56)ccc5c4sc4ccccc45)cc3)ccc2c1 ; c1ccc(-c2ccc3c(sc4ccccc43)c2N(c2cc3ccccc3c3ccccc23)c2cccc3c2oc2ccccc23)cc1]

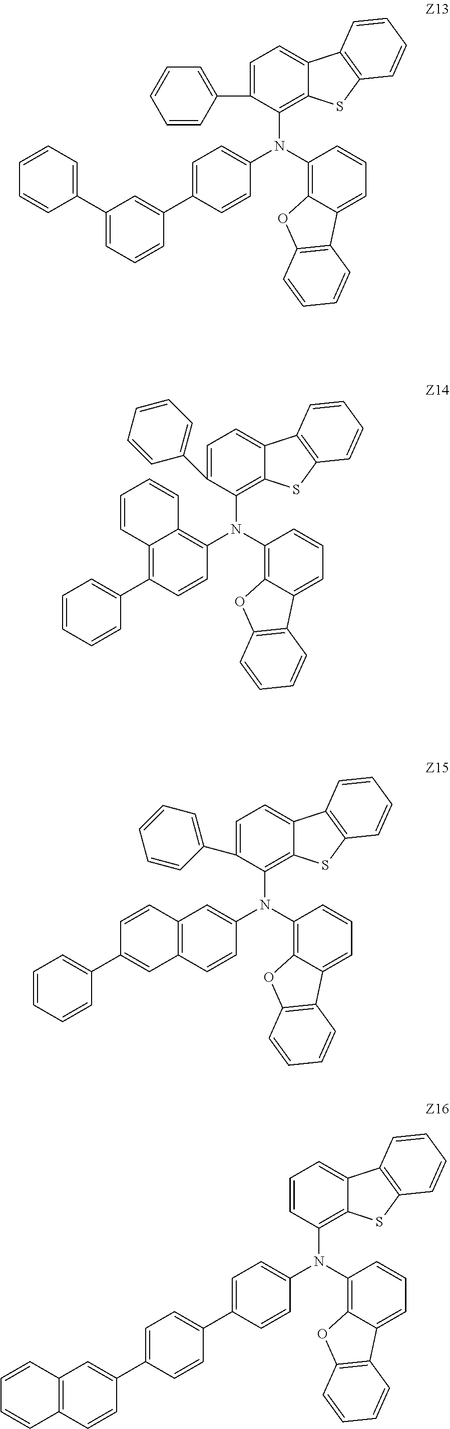 [SMILES: c1ccc(-c2cccc(-c3ccc(N(c4cccc5c4oc4ccccc45)c4c(-c5ccccc5)ccc5c4sc4ccccc45)cc3)c2)cc1 ; c1ccc(-c2ccc3c(sc4ccccc43)c2N(c2ccc(-c3ccccc3)c3ccccc23)c2cccc3c2oc2ccccc23)cc1 ; c1ccc(-c2ccc3cc(N(c4cccc5c4oc4ccccc45)c4c(-c5ccccc5)ccc5c4sc4ccccc45)ccc3c2)cc1 ; c1ccc2cc(-c3ccc(-c4ccc(N(c5cccc6c5oc5ccccc56)c5cccc6c5sc5ccccc56)cc4)cc3)ccc2c1]

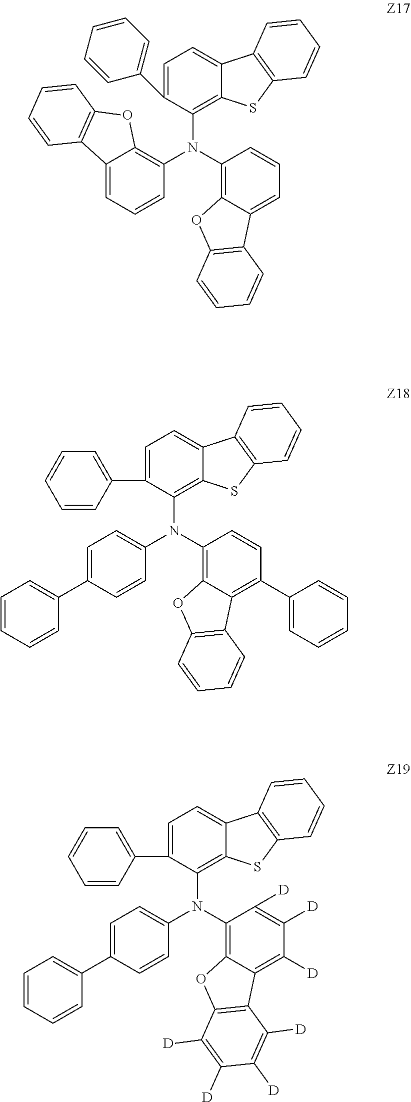 [SMILES: c1ccc(-c2ccc3c(sc4ccccc43)c2N(c2cccc3c2oc2ccccc23)c2cccc3c2oc2ccccc23)cc1 ; c1ccc(-c2ccc(N(c3ccc(-c4ccccc4)c4c3oc3ccccc34)c3c(-c4ccccc4)ccc4c3sc3ccccc34)cc2)cc1 ; [2H]c1c([2H])c([2H])c2c(oc3c(N(c4ccc(-c5ccccc5)cc4)c4c(-c5ccccc5)ccc5c4sc4ccccc45)c([2H])c([2H])c([2H])c32)c1[2H]]

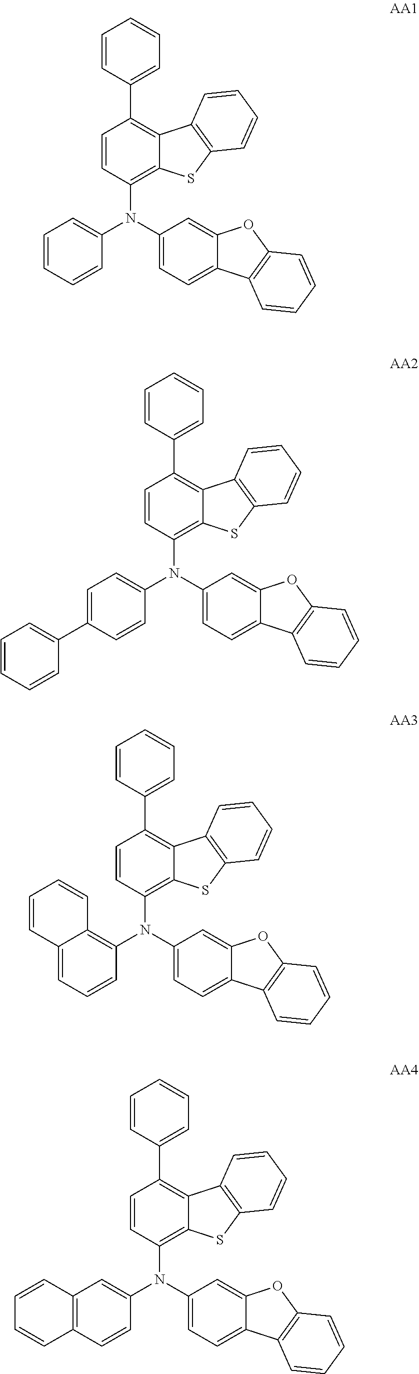 [SMILES: c1ccc(-c2ccc(N(c3ccccc3)c3ccc4c(c3)oc3ccccc34)c3sc4ccccc4c23)cc1 ; c1ccc(-c2ccc(N(c3ccc4c(c3)oc3ccccc34)c3ccc(-c4ccccc4)c4c3sc3ccccc34)cc2)cc1 ; c1ccc(-c2ccc(N(c3ccc4c(c3)oc3ccccc34)c3cccc4ccccc34)c3sc4ccccc4c23)cc1 ; c1ccc(-c2ccc(N(c3ccc4ccccc4c3)c3ccc4c(c3)oc3ccccc34)c3sc4ccccc4c23)cc1]

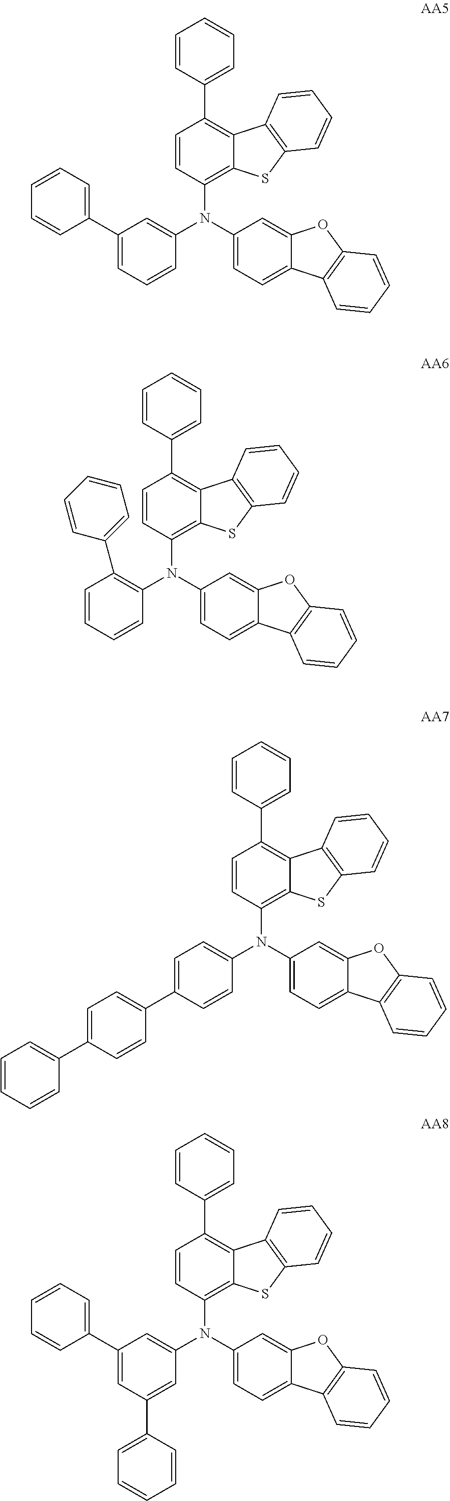 [SMILES: c1ccc(-c2cccc(N(c3ccc4c(c3)oc3ccccc34)c3ccc(-c4ccccc4)c4c3sc3ccccc34)c2)cc1 ; c1ccc(-c2ccccc2N(c2ccc3c(c2)oc2ccccc23)c2ccc(-c3ccccc3)c3c2sc2ccccc23)cc1 ; c1ccc(-c2ccc(-c3ccc(N(c4ccc5c(c4)oc4ccccc45)c4ccc(-c5ccccc5)c5c4sc4ccccc45)cc3)cc2)cc1 ; c1ccc(-c2cc(-c3ccccc3)cc(N(c3ccc4c(c3)oc3ccccc34)c3ccc(-c4ccccc4)c4c3sc3ccccc34)c2)cc1]

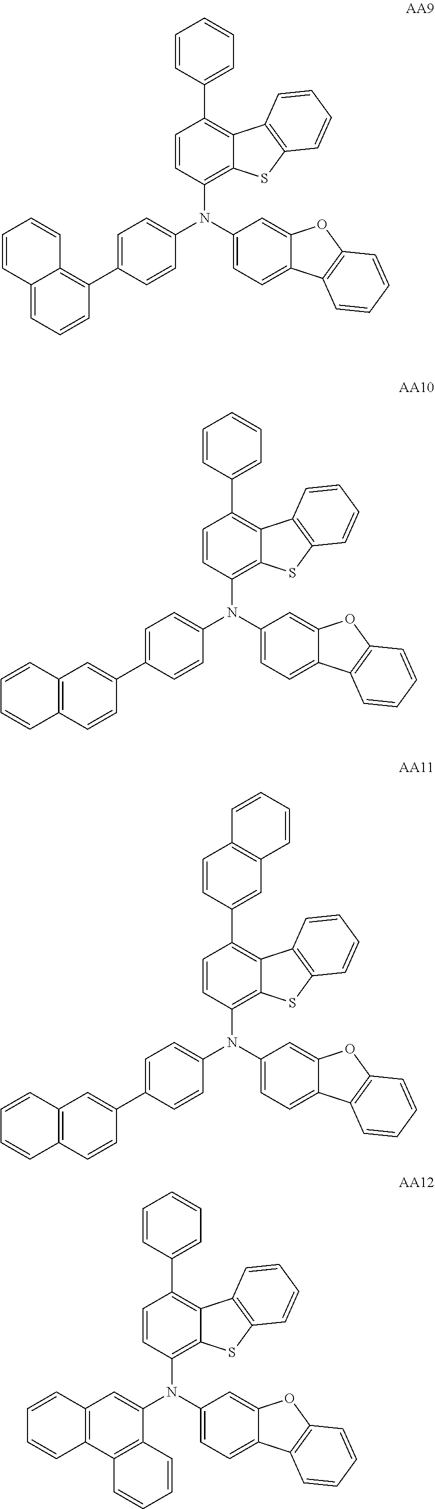 [SMILES: c1ccc(-c2ccc(N(c3ccc(-c4cccc5ccccc45)cc3)c3ccc4c(c3)oc3ccccc34)c3sc4ccccc4c23)cc1 ; c1ccc(-c2ccc(N(c3ccc(-c4ccc5ccccc5c4)cc3)c3ccc4c(c3)oc3ccccc34)c3sc4ccccc4c23)cc1 ; c1ccc2cc(-c3ccc(N(c4ccc5c(c4)oc4ccccc45)c4ccc(-c5ccc6ccccc6c5)c5c4sc4ccccc45)cc3)ccc2c1 ; c1ccc(-c2ccc(N(c3ccc4c(c3)oc3ccccc34)c3cc4ccccc4c4ccccc34)c3sc4ccccc4c23)cc1]

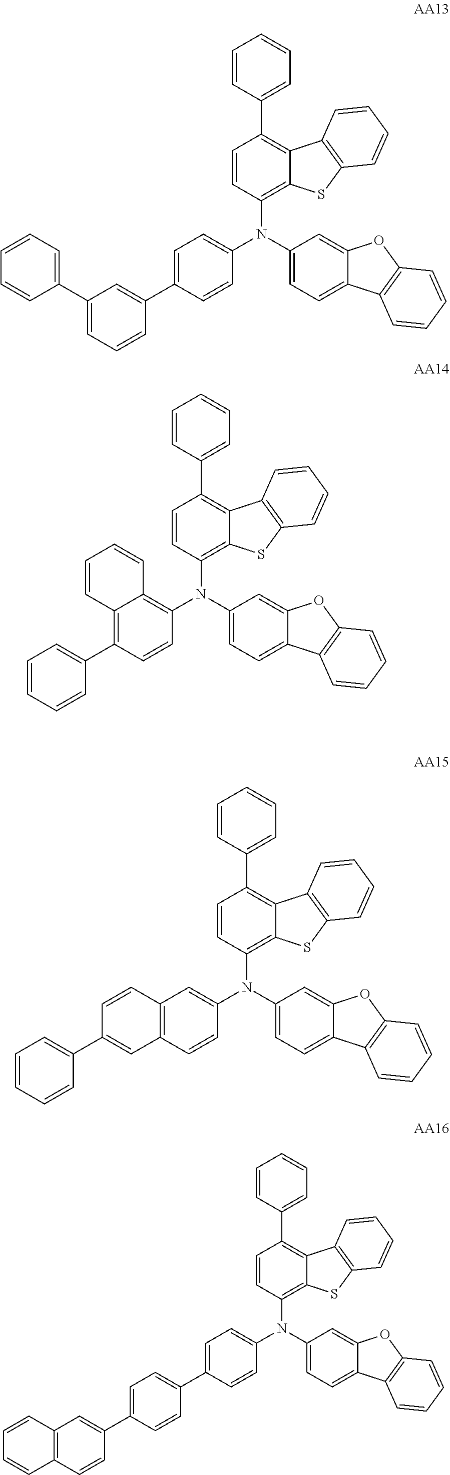 [SMILES: c1ccc(-c2cccc(-c3ccc(N(c4ccc5c(c4)oc4ccccc45)c4ccc(-c5ccccc5)c5c4sc4ccccc45)cc3)c2)cc1 ; c1ccc(-c2ccc(N(c3ccc4c(c3)oc3ccccc34)c3ccc(-c4ccccc4)c4c3sc3ccccc34)c3ccccc23)cc1 ; c1ccc(-c2ccc3cc(N(c4ccc5c(c4)oc4ccccc45)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc4c(c3)oc3ccccc34)c3sc4ccccc4c23)cc1]

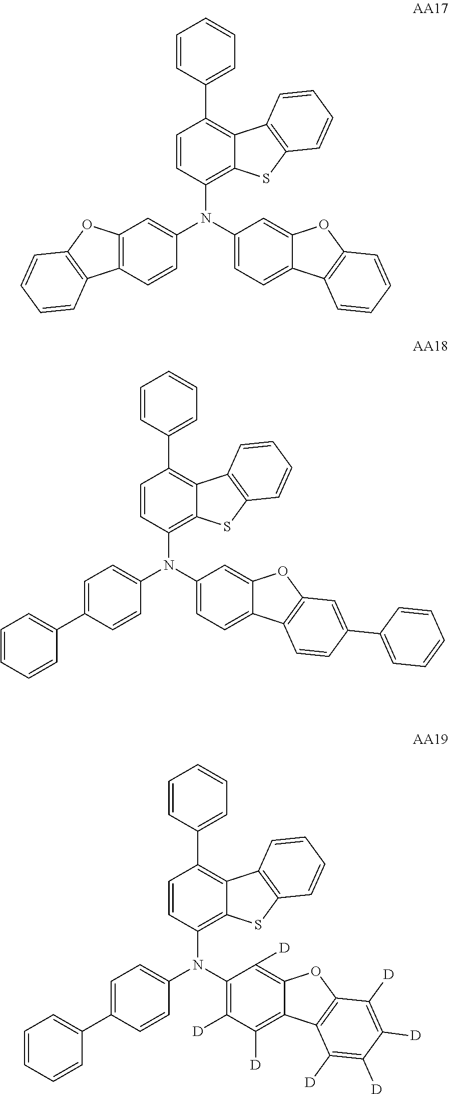 [SMILES: c1ccc(-c2ccc(N(c3ccc4c(c3)oc3ccccc34)c3ccc4c(c3)oc3ccccc34)c3sc4ccccc4c23)cc1 ; c1ccc(-c2ccc(N(c3ccc4c(c3)oc3cc(-c5ccccc5)ccc34)c3ccc(-c4ccccc4)c4c3sc3ccccc34)cc2)cc1 ; [2H]c1c([2H])c([2H])c2c(oc3c([2H])c(N(c4ccc(-c5ccccc5)cc4)c4ccc(-c5ccccc5)c5c4sc4ccccc45)c([2H])c([2H])c32)c1[2H]]

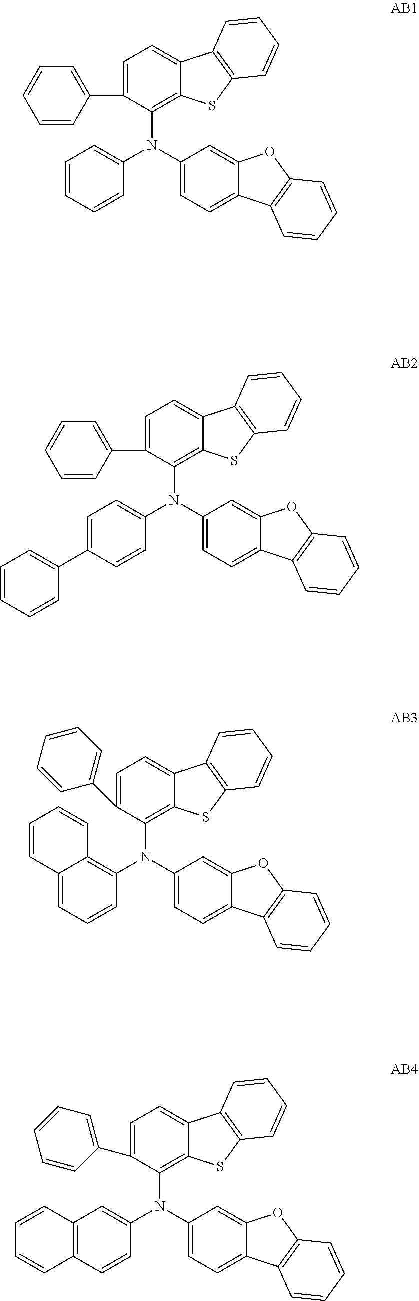 [SMILES: c1ccc(-c2ccc3c(sc4ccccc43)c2N(c2ccccc2)c2ccc3c(c2)oc2ccccc23)cc1 ; c1ccc(-c2ccc(N(c3ccc4c(c3)oc3ccccc34)c3c(-c4ccccc4)ccc4c3sc3ccccc34)cc2)cc1 ; c1ccc(-c2ccc3c(sc4ccccc43)c2N(c2ccc3c(c2)oc2ccccc23)c2cccc3ccccc23)cc1 ; c1ccc(-c2ccc3c(sc4ccccc43)c2N(c2ccc3ccccc3c2)c2ccc3c(c2)oc2ccccc23)cc1]

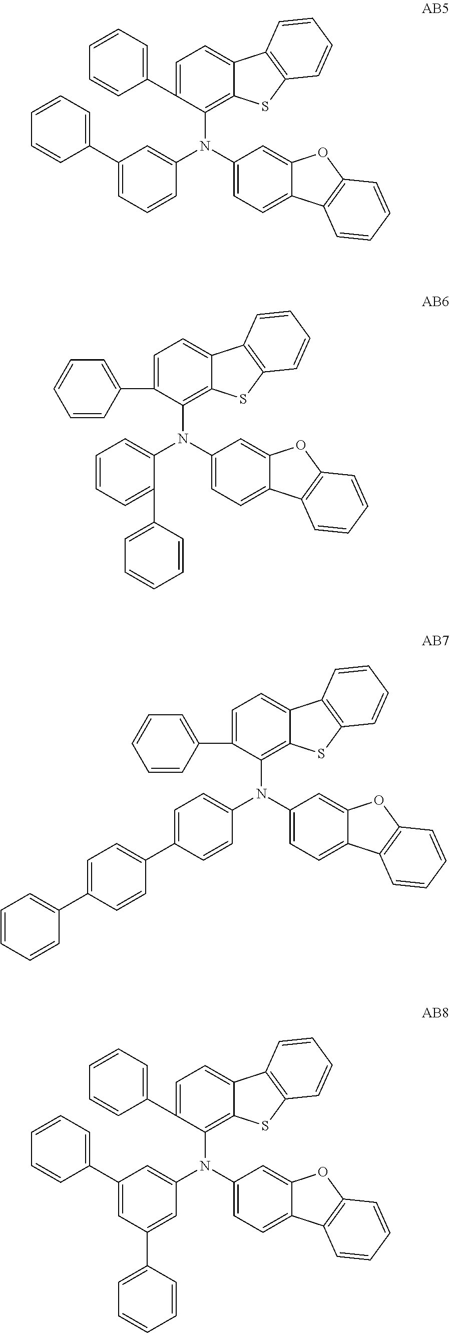 [SMILES: c1ccc(-c2cccc(N(c3ccc4c(c3)oc3ccccc34)c3c(-c4ccccc4)ccc4c3sc3ccccc34)c2)cc1 ; c1ccc(-c2ccccc2N(c2ccc3c(c2)oc2ccccc23)c2c(-c3ccccc3)ccc3c2sc2ccccc23)cc1 ; c1ccc(-c2ccc(-c3ccc(N(c4ccc5c(c4)oc4ccccc45)c4c(-c5ccccc5)ccc5c4sc4ccccc45)cc3)cc2)cc1 ; c1ccc(-c2cc(-c3ccccc3)cc(N(c3ccc4c(c3)oc3ccccc34)c3c(-c4ccccc4)ccc4c3sc3ccccc34)c2)cc1]

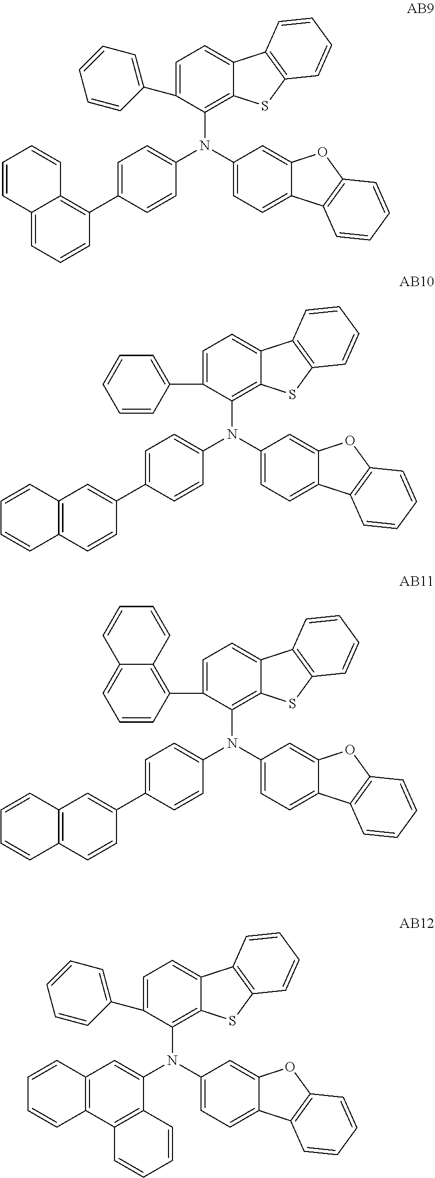 [SMILES: c1ccc(-c2ccc3c(sc4ccccc43)c2N(c2ccc(-c3cccc4ccccc34)cc2)c2ccc3c(c2)oc2ccccc23)cc1 ; c1ccc(-c2ccc3c(sc4ccccc43)c2N(c2ccc(-c3ccc4ccccc4c3)cc2)c2ccc3c(c2)oc2ccccc23)cc1 ; c1ccc2cc(-c3ccc(N(c4ccc5c(c4)oc4ccccc45)c4c(-c5cccc6ccccc56)ccc5c4sc4ccccc45)cc3)ccc2c1 ; c1ccc(-c2ccc3c(sc4ccccc43)c2N(c2ccc3c(c2)oc2ccccc23)c2cc3ccccc3c3ccccc23)cc1]

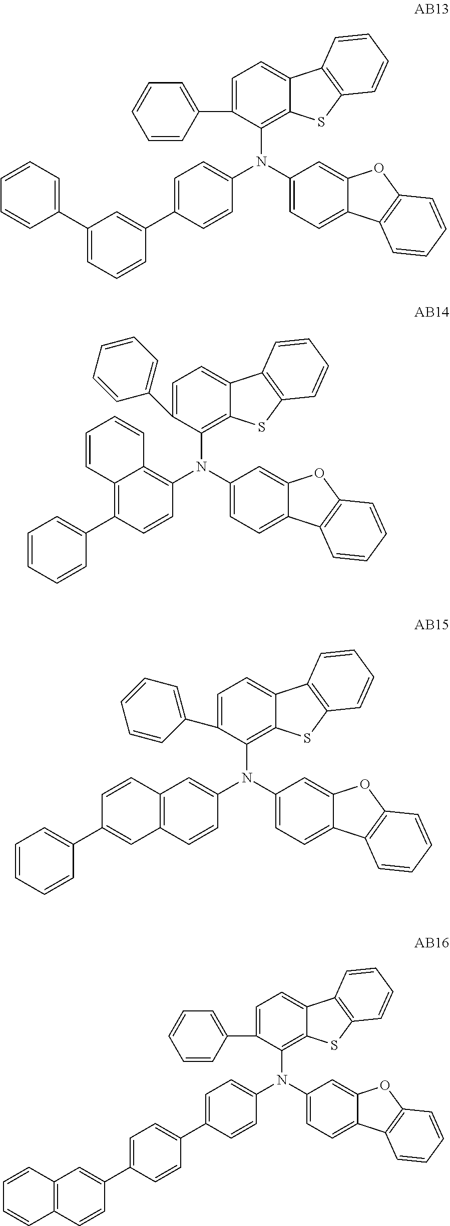 [SMILES: c1ccc(-c2cccc(-c3ccc(N(c4ccc5c(c4)oc4ccccc45)c4c(-c5ccccc5)ccc5c4sc4ccccc45)cc3)c2)cc1 ; c1ccc(-c2ccc3c(sc4ccccc43)c2N(c2ccc3c(c2)oc2ccccc23)c2ccc(-c3ccccc3)c3ccccc23)cc1 ; c1ccc(-c2ccc3cc(N(c4ccc5c(c4)oc4ccccc45)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc3c(c2)oc2ccccc23)cc1]

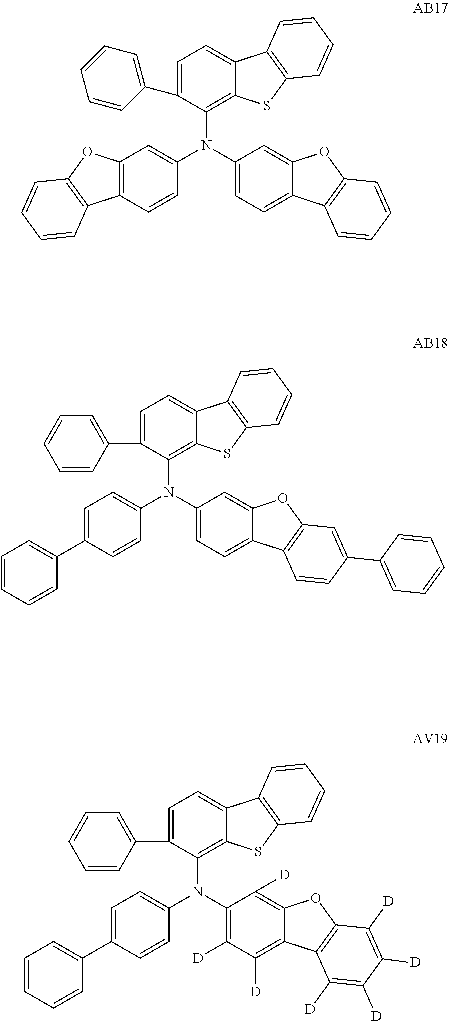 [SMILES: c1ccc(-c2ccc3c(sc4ccccc43)c2N(c2ccc3c(c2)oc2ccccc23)c2ccc3c(c2)oc2ccccc23)cc1 ; c1ccc(-c2ccc(N(c3ccc4c(c3)oc3cc(-c5ccccc5)ccc34)c3c(-c4ccccc4)ccc4c3sc3ccccc34)cc2)cc1 ; [2H]c1c([2H])c([2H])c2c(oc3c([2H])c(N(c4ccc(-c5ccccc5)cc4)c4c(-c5ccccc5)ccc5c4sc4ccccc45)c([2H])c([2H])c32)c1[2H]]

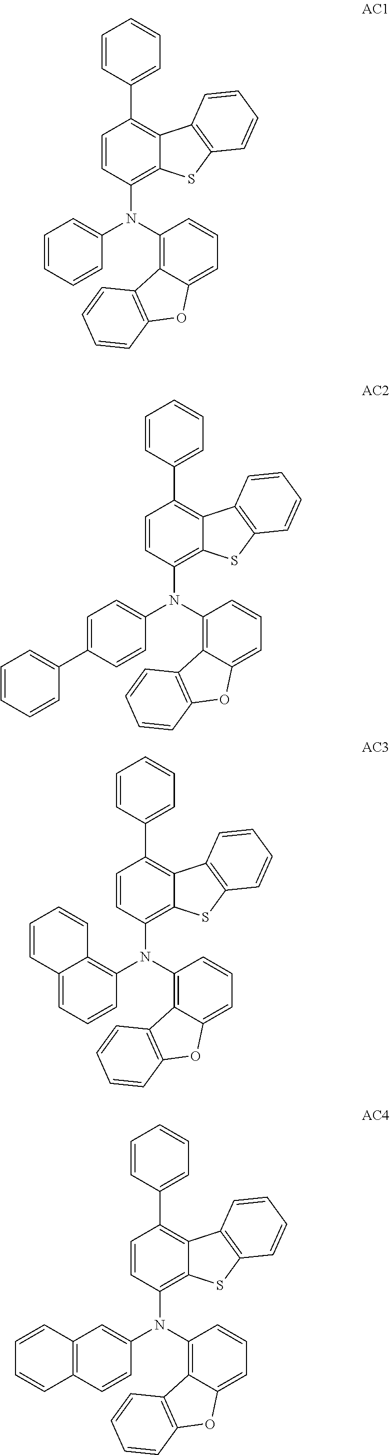

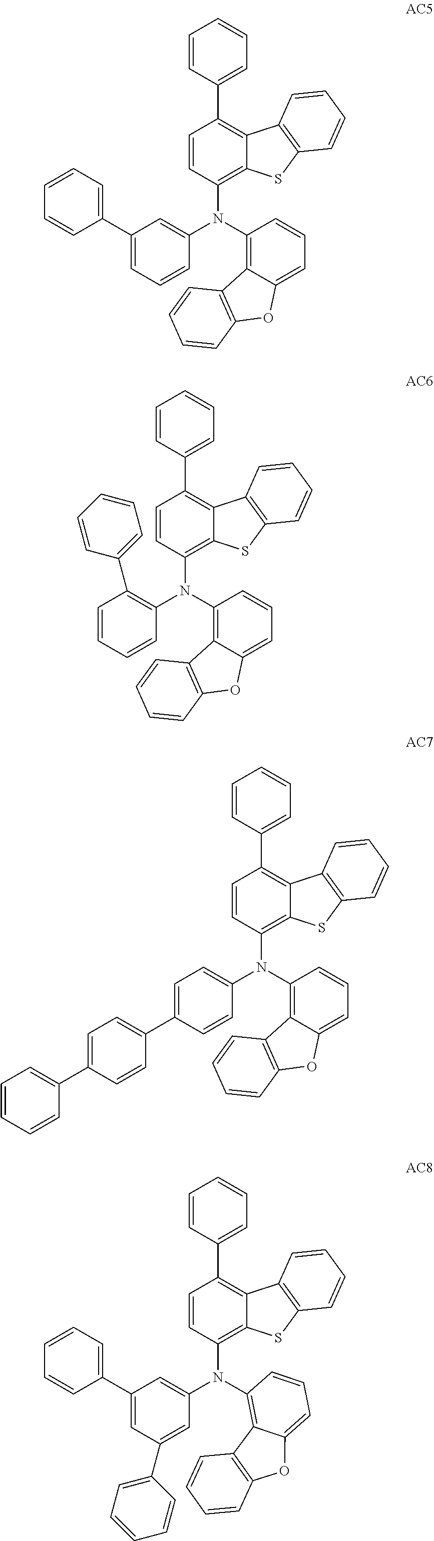

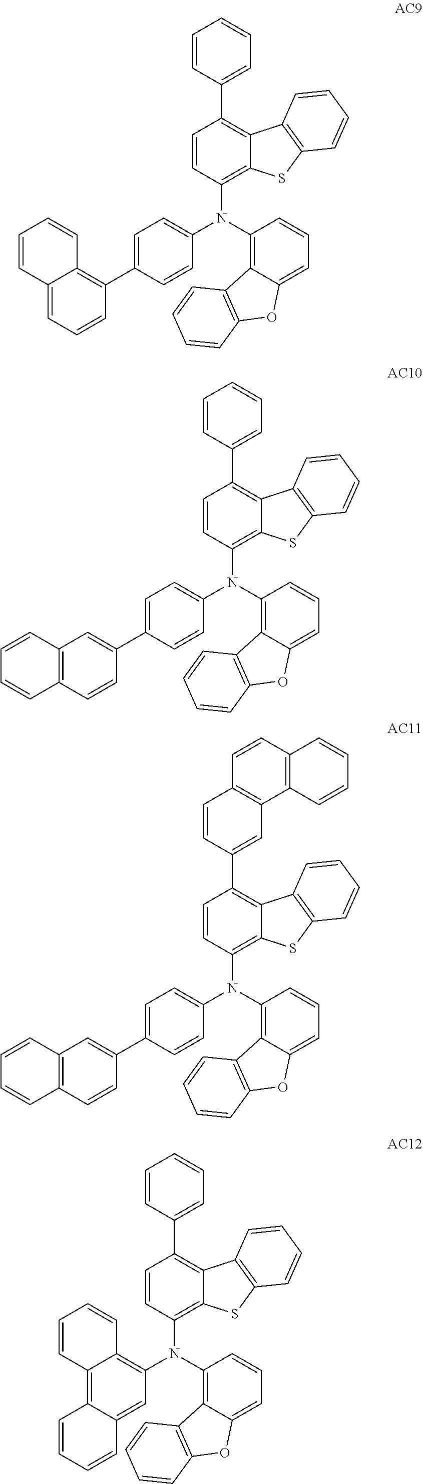

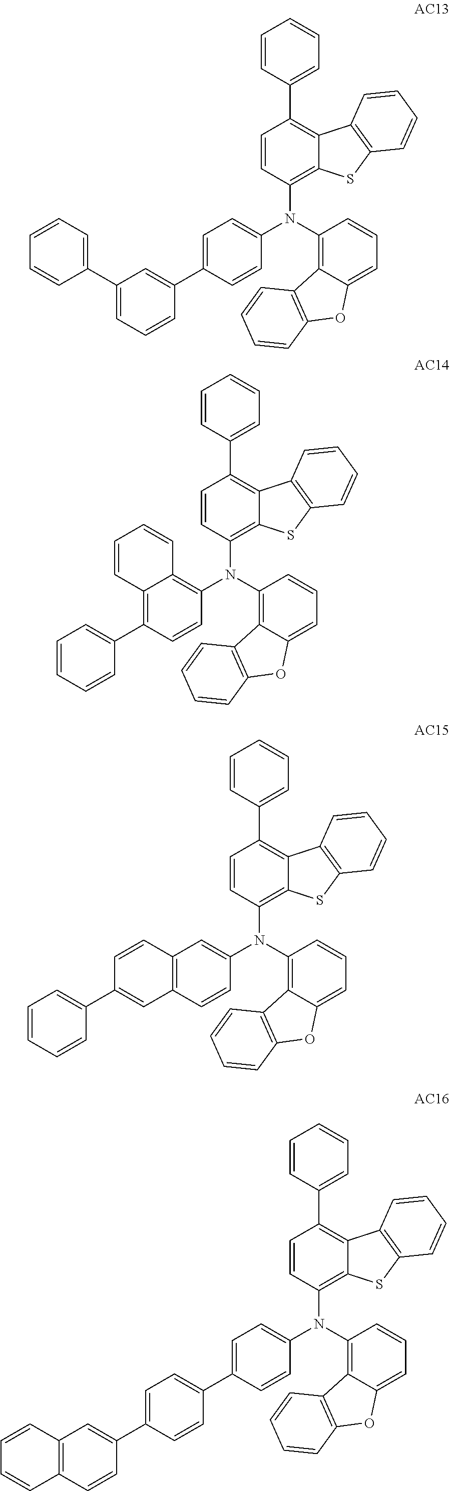

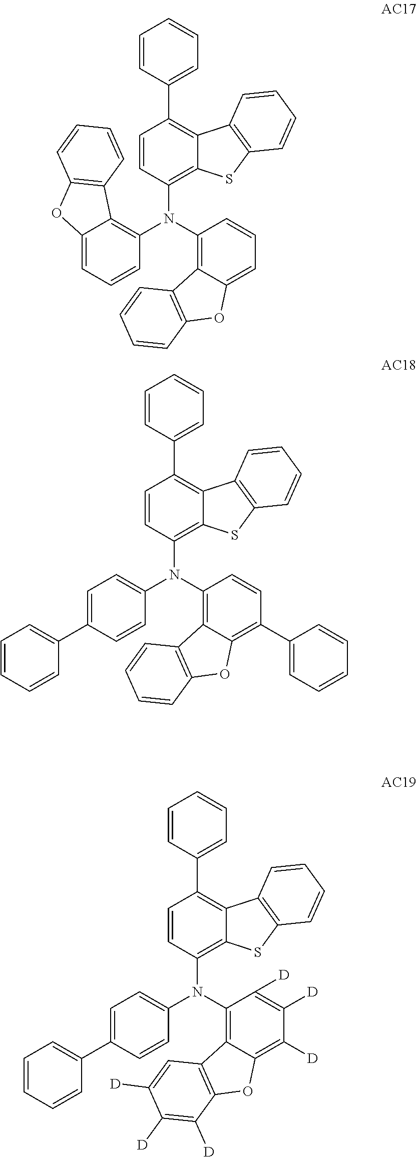

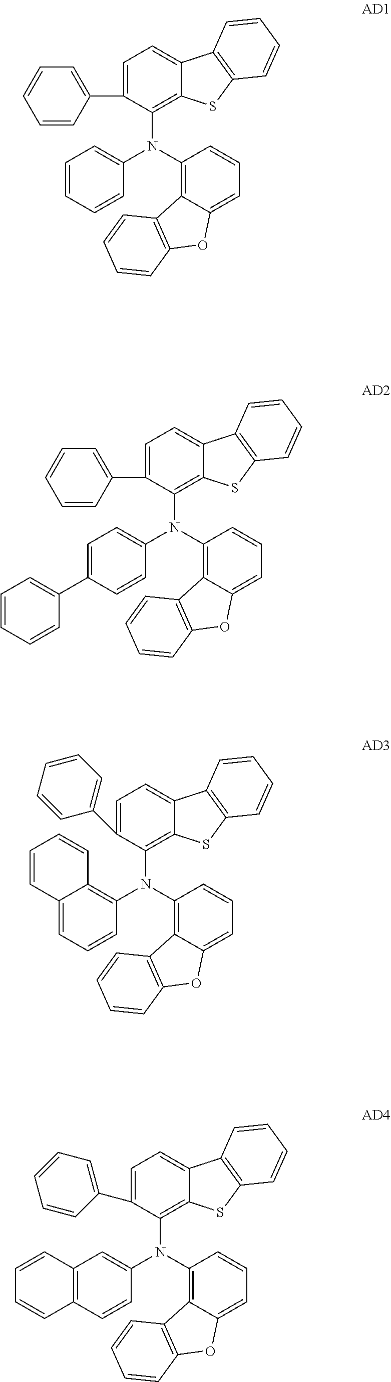 [SMILES: c1ccc(-c2ccc3c(sc4ccccc43)c2N(c2ccccc2)c2cccc3oc4ccccc4c23)cc1 ; c1ccc(-c2ccc(N(c3c(-c4ccccc4)ccc4c3sc3ccccc34)c3cccc4oc5ccccc5c34)cc2)cc1 ; c1ccc(-c2ccc3c(sc4ccccc43)c2N(c2cccc3ccccc23)c2cccc3oc4ccccc4c23)cc1 ; c1ccc(-c2ccc3c(sc4ccccc43)c2N(c2ccc3ccccc3c2)c2cccc3oc4ccccc4c23)cc1]

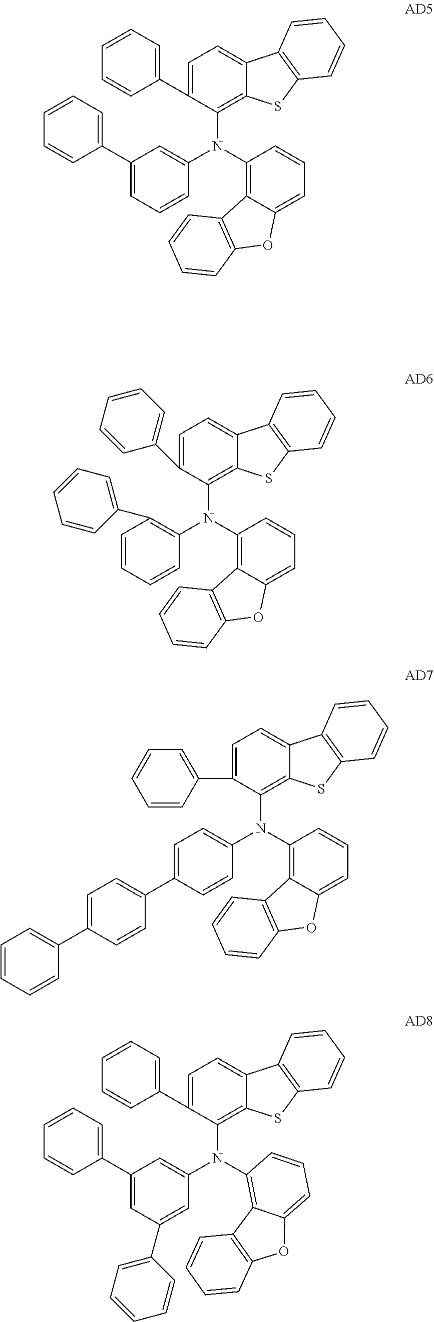 [SMILES: c1ccc(-c2cccc(N(c3c(-c4ccccc4)ccc4c3sc3ccccc34)c3cccc4oc5ccccc5c34)c2)cc1 ; c1ccc(-c2ccccc2N(c2c(-c3ccccc3)ccc3c2sc2ccccc23)c2cccc3oc4ccccc4c23)cc1 ; c1ccc(-c2ccc(-c3ccc(N(c4c(-c5ccccc5)ccc5c4sc4ccccc45)c4cccc5oc6ccccc6c45)cc3)cc2)cc1 ; c1ccc(-c2cc(-c3ccccc3)cc(N(c3c(-c4ccccc4)ccc4c3sc3ccccc34)c3cccc4oc5ccccc5c34)c2)cc1]

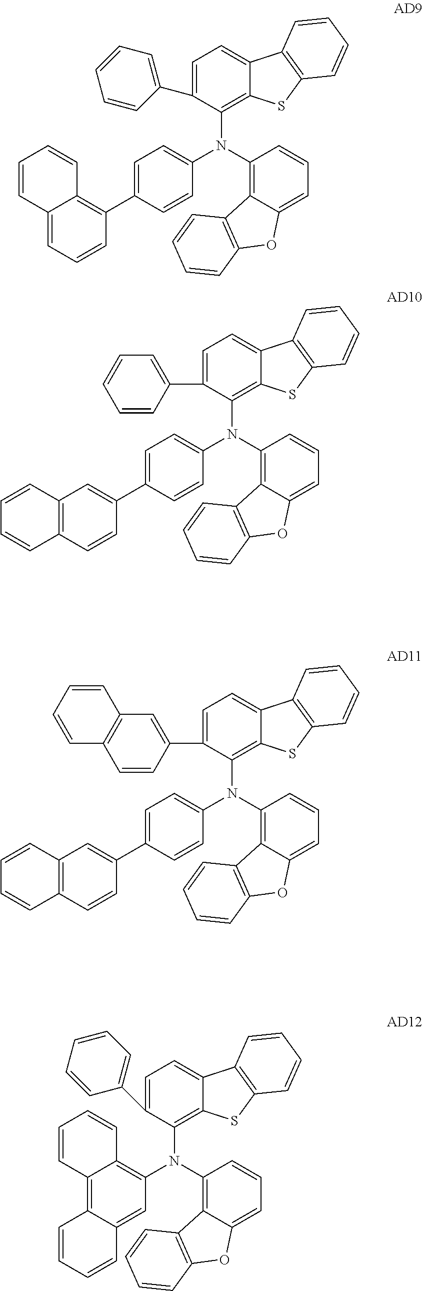 [SMILES: c1ccc(-c2ccc3c(sc4ccccc43)c2N(c2ccc(-c3cccc4ccccc34)cc2)c2cccc3oc4ccccc4c23)cc1 ; c1ccc(-c2ccc3c(sc4ccccc43)c2N(c2ccc(-c3ccc4ccccc4c3)cc2)c2cccc3oc4ccccc4c23)cc1 ; c1ccc2cc(-c3ccc(N(c4c(-c5ccc6ccccc6c5)ccc5c4sc4ccccc45)c4cccc5oc6ccccc6c45)cc3)ccc2c1 ; c1ccc(-c2ccc3c(sc4ccccc43)c2N(c2cc3ccccc3c3ccccc23)c2cccc3oc4ccccc4c23)cc1]

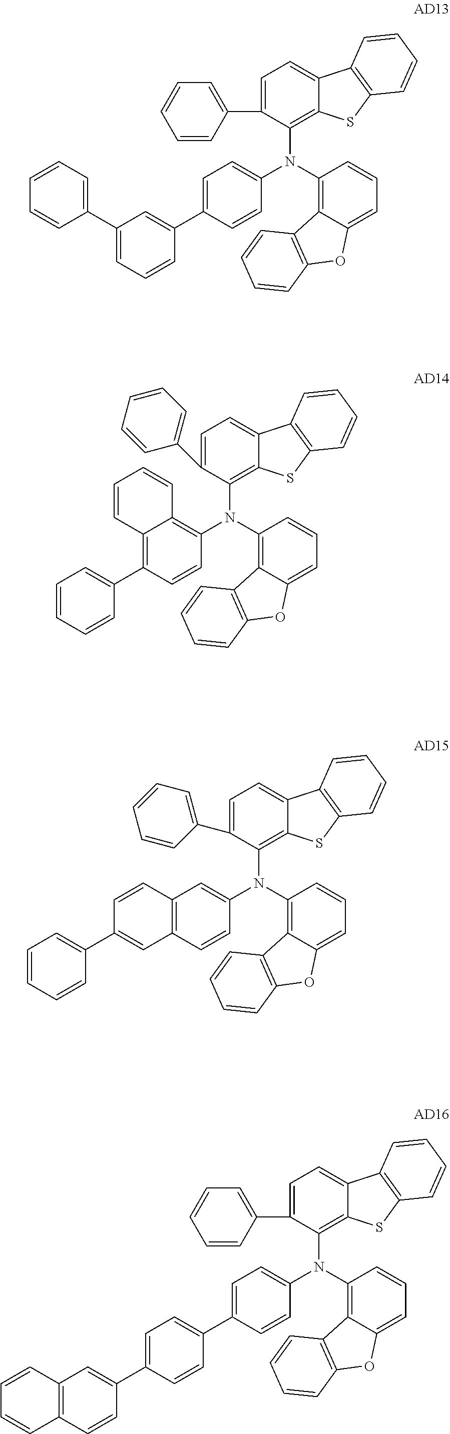 [SMILES: c1ccc(-c2cccc(-c3ccc(N(c4c(-c5ccccc5)ccc5c4sc4ccccc45)c4cccc5oc6ccccc6c45)cc3)c2)cc1 ; c1ccc(-c2ccc3c(sc4ccccc43)c2N(c2ccc(-c3ccccc3)c3ccccc23)c2cccc3oc4ccccc4c23)cc1 ; c1ccc(-c2ccc3cc(N(c4c(-c5ccccc5)ccc5c4sc4ccccc45)c4cccc5oc6ccccc6c45)ccc3c2)cc1 ; c1ccc(-c2ccc3c(sc4ccccc43)c2N(c2ccc(-c3ccc(-c4ccc5ccccc5c4)cc3)cc2)c2cccc3oc4ccccc4c23)cc1]

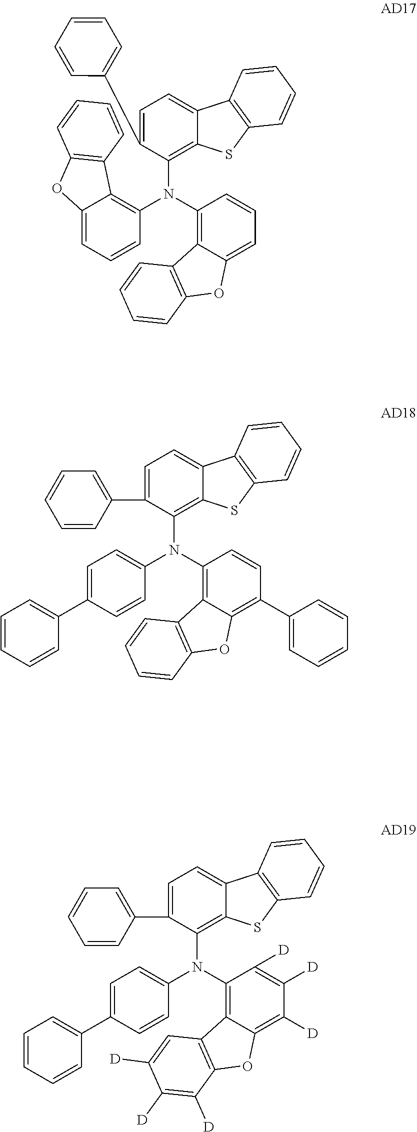 [SMILES: c1ccc(-c2ccc3c(sc4ccccc43)c2N(c2cccc3oc4ccccc4c23)c2cccc3oc4ccccc4c23)cc1 ; c1ccc(-c2ccc(N(c3c(-c4ccccc4)ccc4c3sc3ccccc34)c3ccc(-c4ccccc4)c4oc5ccccc5c34)cc2)cc1 ; [2H]c1cc2c(oc3c([2H])c([2H])c([2H])c(N(c4ccc(-c5ccccc5)cc4)c4c(-c5ccccc5)ccc5c4sc4ccccc45)c32)c([2H])c1[2H]]

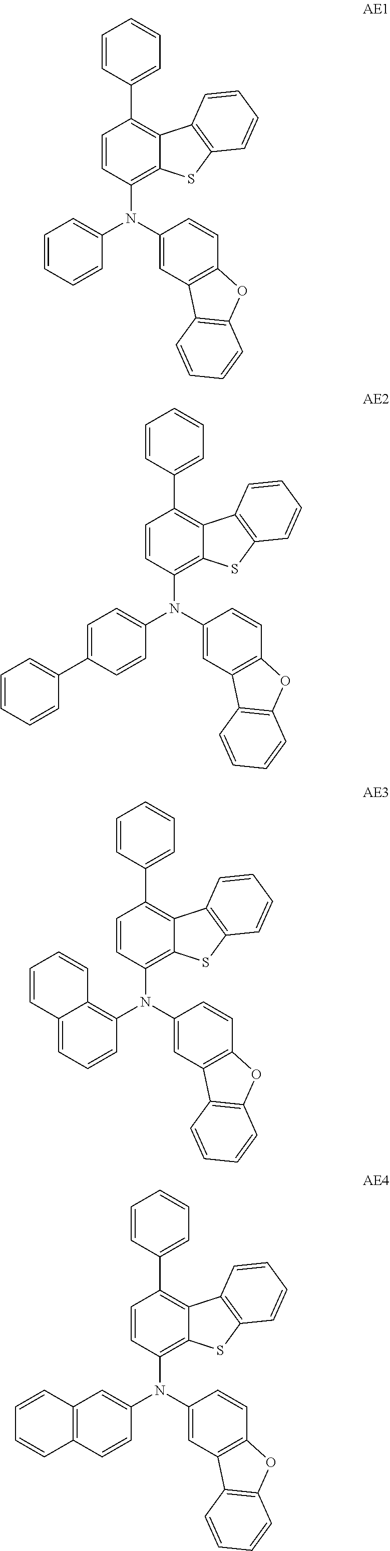

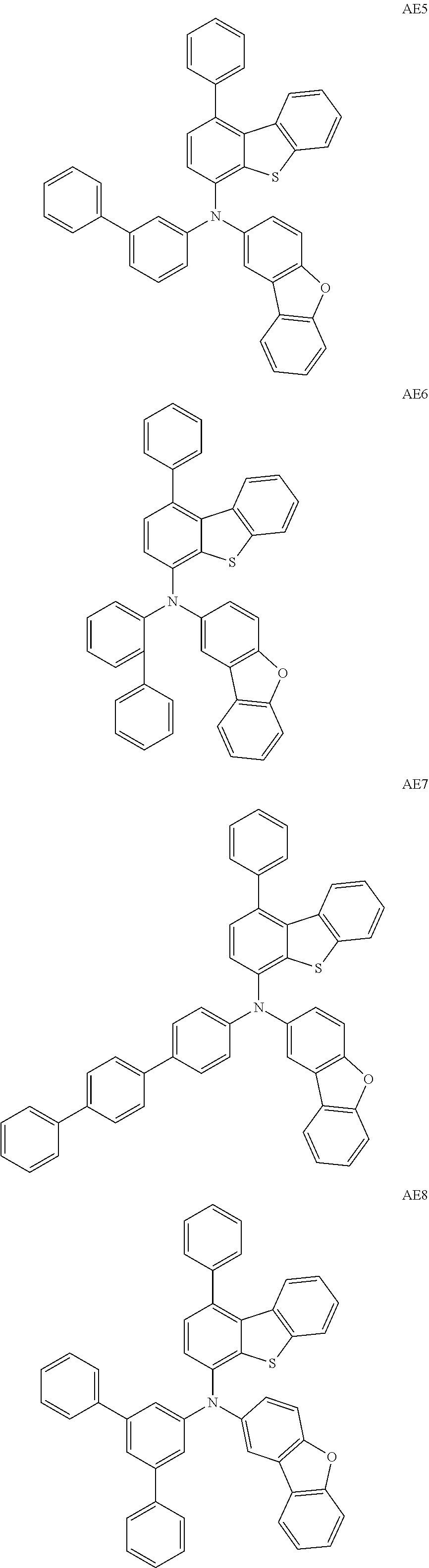 [SMILES: c1ccc(-c2cccc(N(c3ccc4oc5ccccc5c4c3)c3ccc(-c4ccccc4)c4c3sc3ccccc34)c2)cc1 ; c1ccc(-c2ccccc2N(c2ccc3oc4ccccc4c3c2)c2ccc(-c3ccccc3)c3c2sc2ccccc23)cc1 ; c1ccc(-c2ccc(-c3ccc(N(c4ccc5oc6ccccc6c5c4)c4ccc(-c5ccccc5)c5c4sc4ccccc45)cc3)cc2)cc1 ; c1ccc(-c2cc(-c3ccccc3)cc(N(c3ccc4oc5ccccc5c4c3)c3ccc(-c4ccccc4)c4c3sc3ccccc34)c2)cc1]

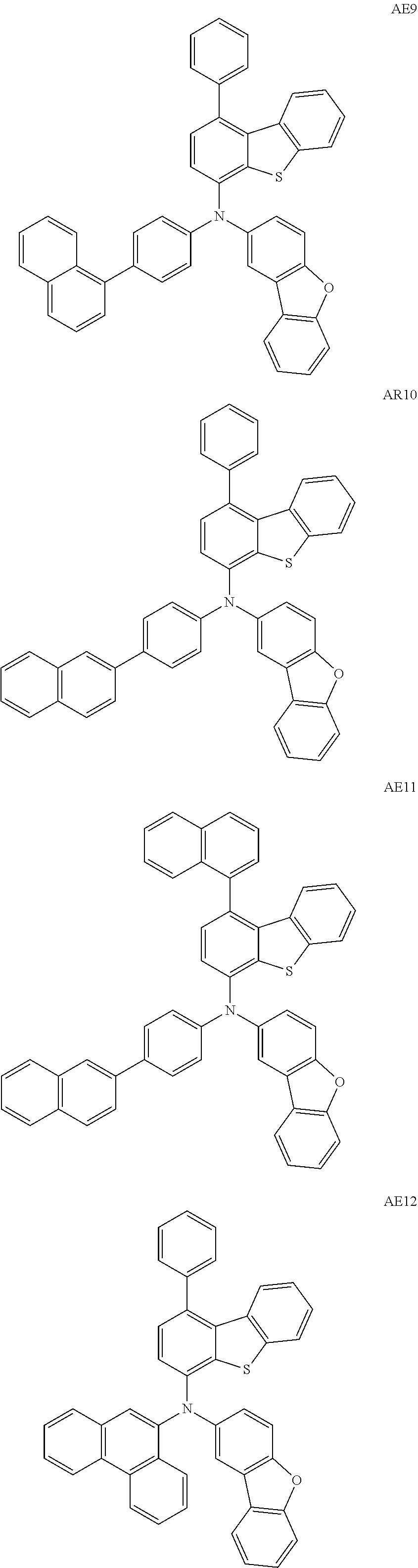 [SMILES: c1ccc(-c2ccc(N(c3ccc(-c4cccc5ccccc45)cc3)c3ccc4oc5ccccc5c4c3)c3sc4ccccc4c23)cc1 ; c1ccc(-c2ccc(N(c3ccc(-c4ccc5ccccc5c4)cc3)c3ccc4oc5ccccc5c4c3)c3sc4ccccc4c23)cc1 ; c1ccc2cc(-c3ccc(N(c4ccc5oc6ccccc6c5c4)c4ccc(-c5cccc6ccccc56)c5c4sc4ccccc45)cc3)ccc2c1 ; c1ccc(-c2ccc(N(c3ccc4oc5ccccc5c4c3)c3cc4ccccc4c4ccccc34)c3sc4ccccc4c23)cc1]

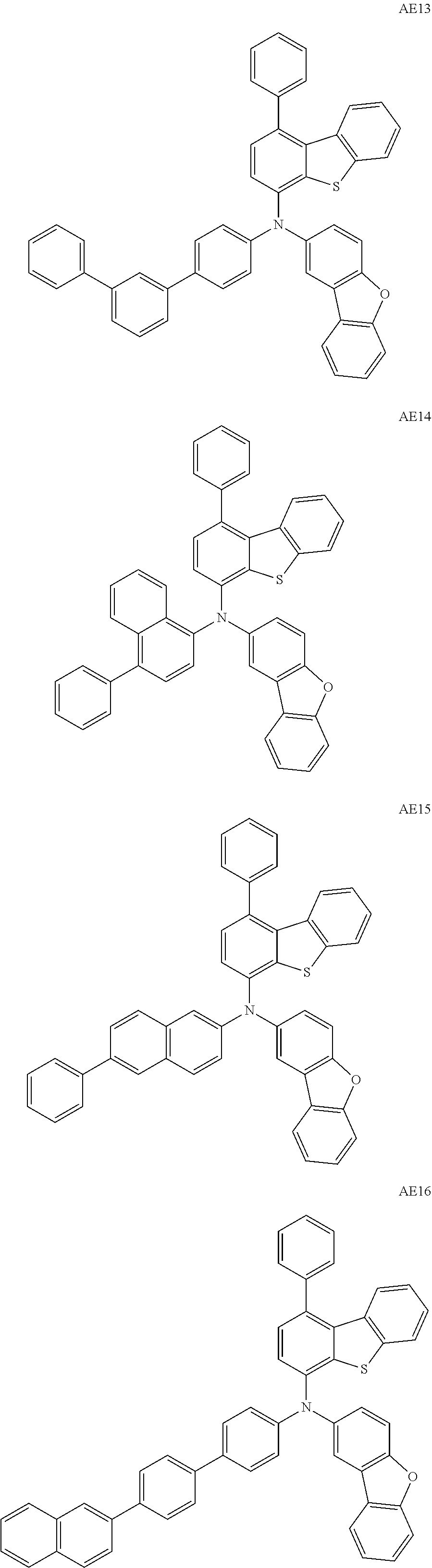 [SMILES: c1ccc(-c2cccc(-c3ccc(N(c4ccc5oc6ccccc6c5c4)c4ccc(-c5ccccc5)c5c4sc4ccccc45)cc3)c2)cc1 ; c1ccc(-c2ccc(N(c3ccc4oc5ccccc5c4c3)c3ccc(-c4ccccc4)c4c3sc3ccccc34)c3ccccc23)cc1 ; c1ccc(-c2ccc3cc(N(c4ccc5oc6ccccc6c5c4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc4oc5ccccc5c4c3)c3sc4ccccc4c23)cc1]

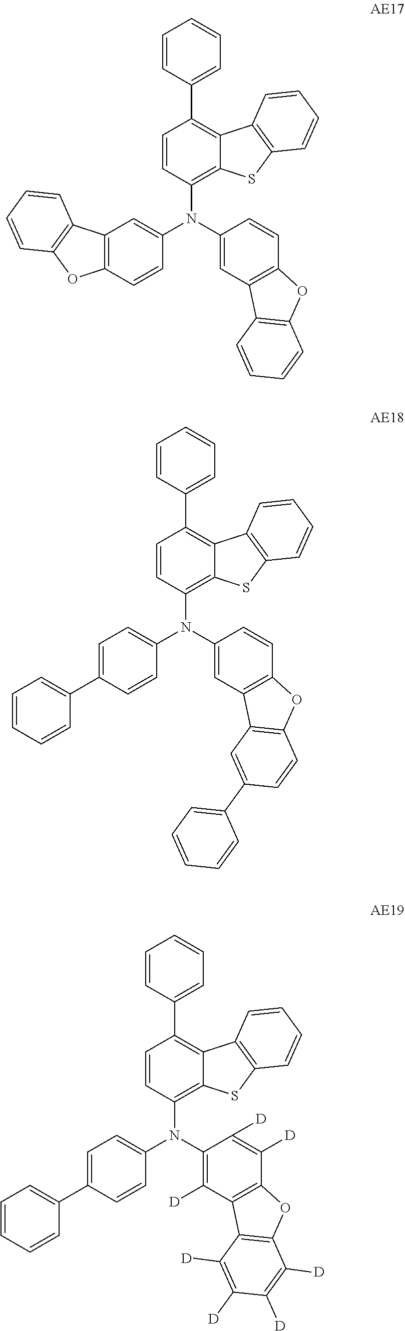 [SMILES: c1ccc(-c2ccc(N(c3ccc4oc5ccccc5c4c3)c3ccc4oc5ccccc5c4c3)c3sc4ccccc4c23)cc1 ; c1ccc(-c2ccc(N(c3ccc4oc5ccc(-c6ccccc6)cc5c4c3)c3ccc(-c4ccccc4)c4c3sc3ccccc34)cc2)cc1 ; [2H]c1c([2H])c([2H])c2c(oc3c([2H])c([2H])c(N(c4ccc(-c5ccccc5)cc4)c4ccc(-c5ccccc5)c5c4sc4ccccc45)c([2H])c32)c1[2H]]

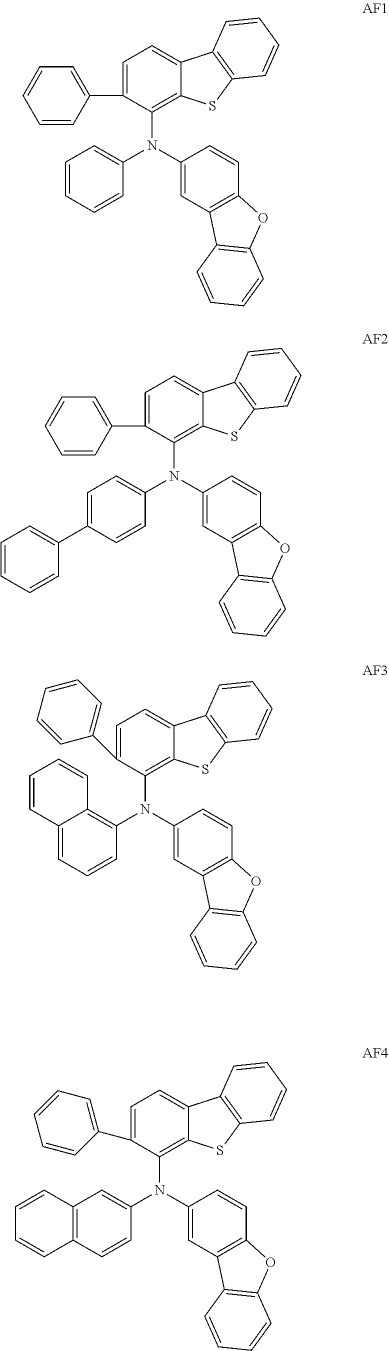

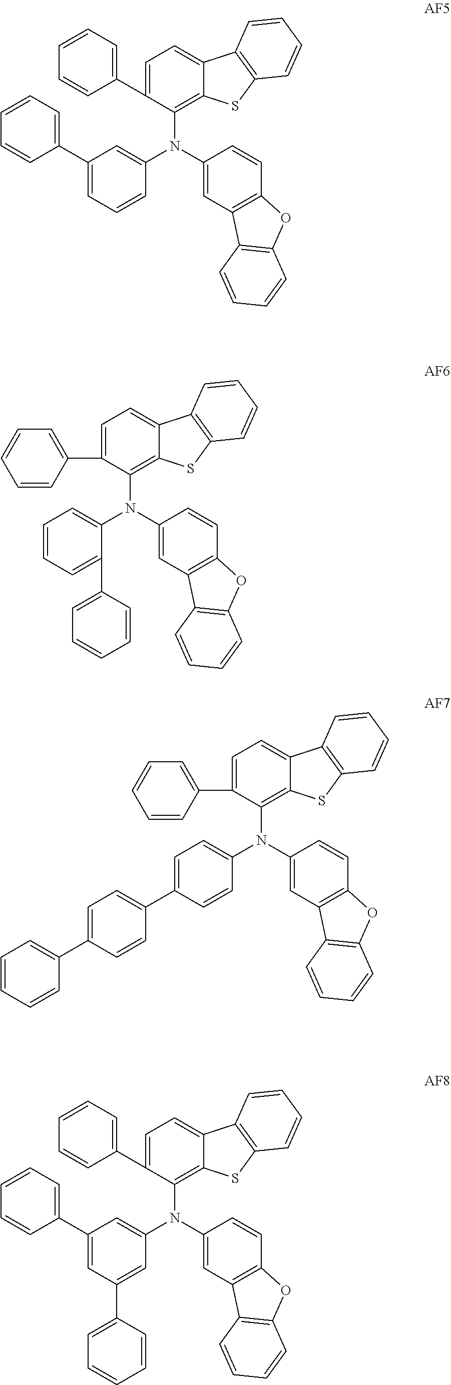 [SMILES: c1ccc(-c2cccc(N(c3ccc4oc5ccccc5c4c3)c3c(-c4ccccc4)ccc4c3sc3ccccc34)c2)cc1 ; c1ccc(-c2ccccc2N(c2ccc3oc4ccccc4c3c2)c2c(-c3ccccc3)ccc3c2sc2ccccc23)cc1 ; c1ccc(-c2ccc(-c3ccc(N(c4ccc5oc6ccccc6c5c4)c4c(-c5ccccc5)ccc5c4sc4ccccc45)cc3)cc2)cc1 ; c1ccc(-c2cc(-c3ccccc3)cc(N(c3ccc4oc5ccccc5c4c3)c3c(-c4ccccc4)ccc4c3sc3ccccc34)c2)cc1]

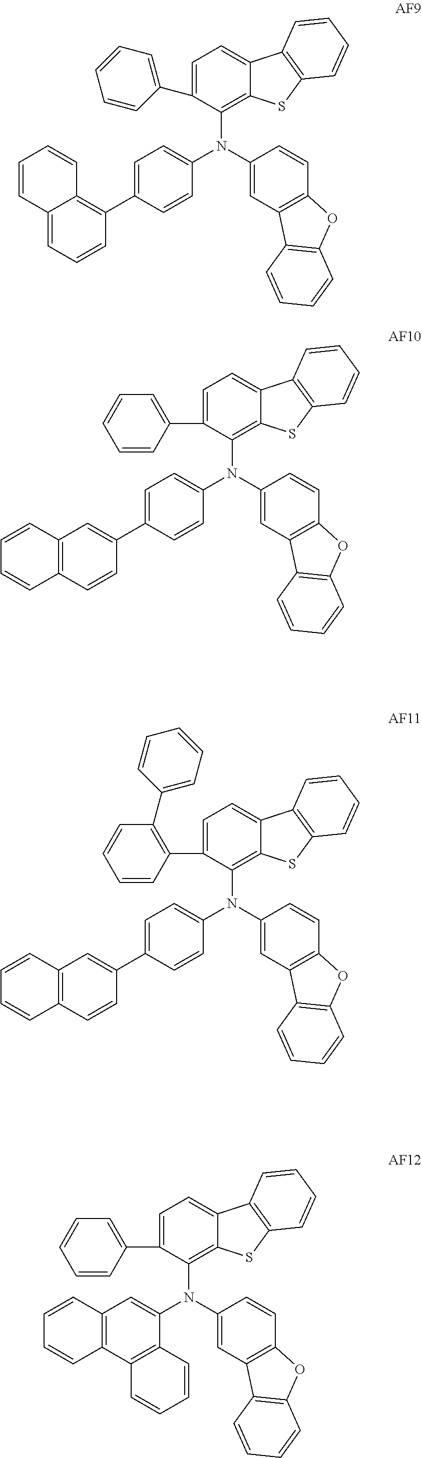

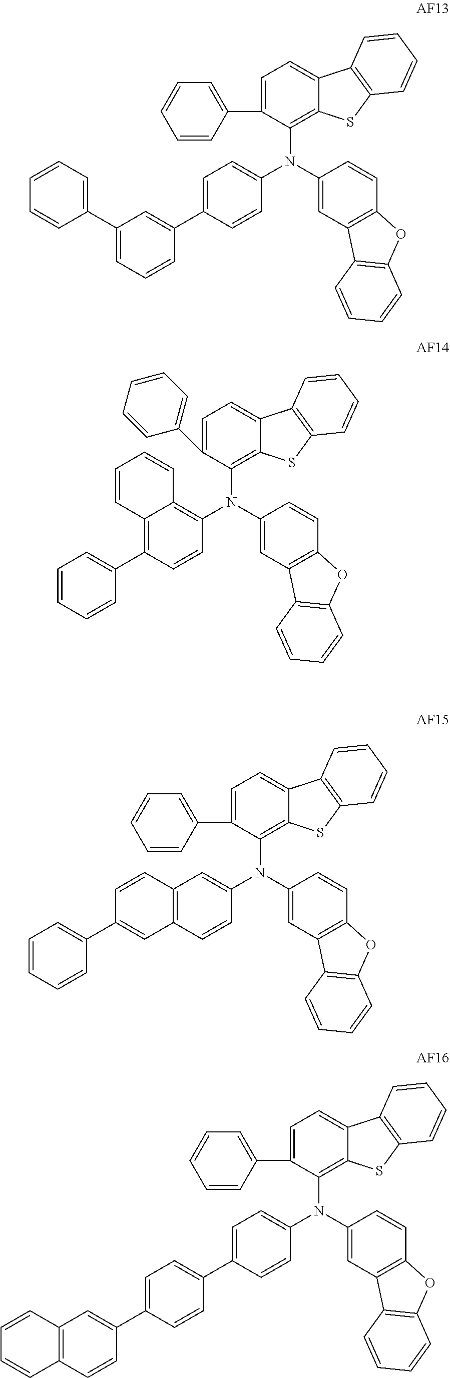 [SMILES: c1ccc(-c2cccc(-c3ccc(N(c4ccc5oc6ccccc6c5c4)c4c(-c5ccccc5)ccc5c4sc4ccccc45)cc3)c2)cc1 ; c1ccc(-c2ccc3c(sc4ccccc43)c2N(c2ccc3oc4ccccc4c3c2)c2ccc(-c3ccccc3)c3ccccc23)cc1 ; c1ccc(-c2ccc3cc(N(c4ccc5oc6ccccc6c5c4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc3oc4ccccc4c3c2)cc1]

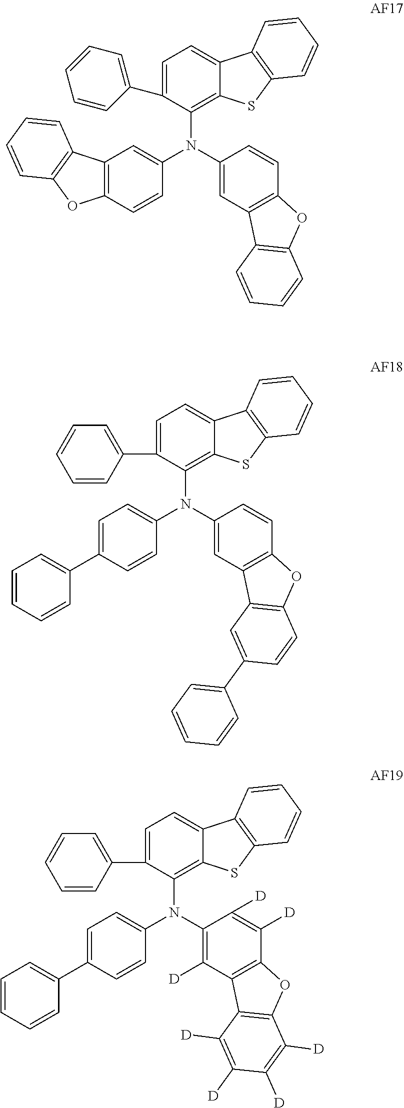 [SMILES: c1ccc(-c2ccc3c(sc4ccccc43)c2N(c2ccc3oc4ccccc4c3c2)c2ccc3oc4ccccc4c3c2)cc1 ; c1ccc(-c2ccc(N(c3ccc4oc5ccc(-c6ccccc6)cc5c4c3)c3c(-c4ccccc4)ccc4c3sc3ccccc34)cc2)cc1 ; [2H]c1c([2H])c([2H])c2c(oc3c([2H])c([2H])c(N(c4ccc(-c5ccccc5)cc4)c4c(-c5ccccc5)ccc5c4sc4ccccc45)c([2H])c32)c1[2H]]

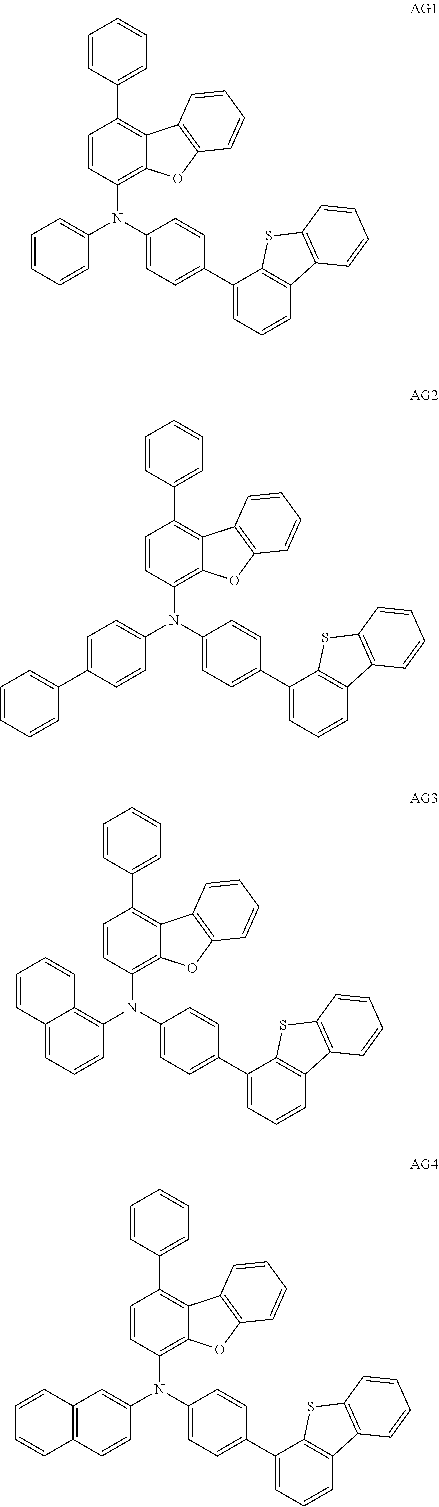 [SMILES: c1ccc(-c2ccc(N(c3ccccc3)c3ccc(-c4cccc5c4sc4ccccc45)cc3)c3oc4ccccc4c23)cc1 ; c1ccc(-c2ccc(N(c3ccc(-c4cccc5c4sc4ccccc45)cc3)c3ccc(-c4ccccc4)c4c3oc3ccccc34)cc2)cc1 ; c1ccc(-c2ccc(N(c3ccc(-c4cccc5c4sc4ccccc45)cc3)c3cccc4ccccc34)c3oc4ccccc4c23)cc1 ; c1ccc(-c2ccc(N(c3ccc(-c4cccc5c4sc4ccccc45)cc3)c3ccc4ccccc4c3)c3oc4ccccc4c23)cc1]

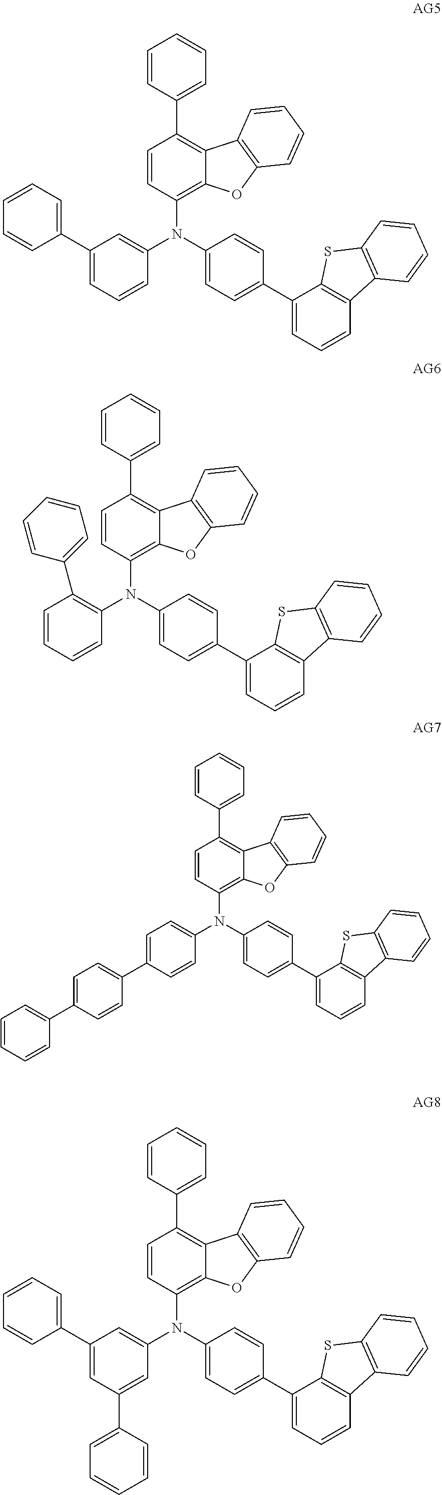 [SMILES: c1ccc(-c2cccc(N(c3ccc(-c4cccc5c4sc4ccccc45)cc3)c3ccc(-c4ccccc4)c4c3oc3ccccc34)c2)cc1 ; c1ccc(-c2ccccc2N(c2ccc(-c3cccc4c3sc3ccccc34)cc2)c2ccc(-c3ccccc3)c3c2oc2ccccc23)cc1 ; c1ccc(-c2ccc(-c3ccc(N(c4ccc(-c5cccc6c5sc5ccccc56)cc4)c4ccc(-c5ccccc5)c5c4oc4ccccc45)cc3)cc2)cc1 ; c1ccc(-c2cc(-c3ccccc3)cc(N(c3ccc(-c4cccc5c4sc4ccccc45)cc3)c3ccc(-c4ccccc4)c4c3oc3ccccc34)c2)cc1]

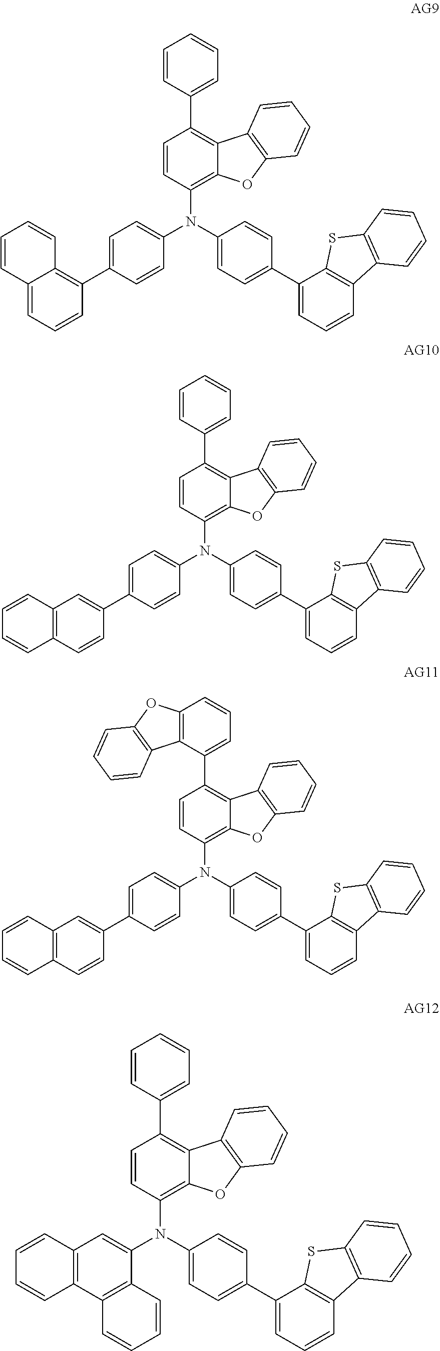 [SMILES: c1ccc(-c2ccc(N(c3ccc(-c4cccc5ccccc45)cc3)c3ccc(-c4cccc5c4sc4ccccc45)cc3)c3oc4ccccc4c23)cc1 ; c1ccc(-c2ccc(N(c3ccc(-c4ccc5ccccc5c4)cc3)c3ccc(-c4cccc5c4sc4ccccc45)cc3)c3oc4ccccc4c23)cc1 ; c1ccc2cc(-c3ccc(N(c4ccc(-c5cccc6c5sc5ccccc56)cc4)c4ccc(-c5cccc6oc7ccccc7c56)c5c4oc4ccccc45)cc3)ccc2c1 ; c1ccc(-c2ccc(N(c3ccc(-c4cccc5c4sc4ccccc45)cc3)c3cc4ccccc4c4ccccc34)c3oc4ccccc4c23)cc1]

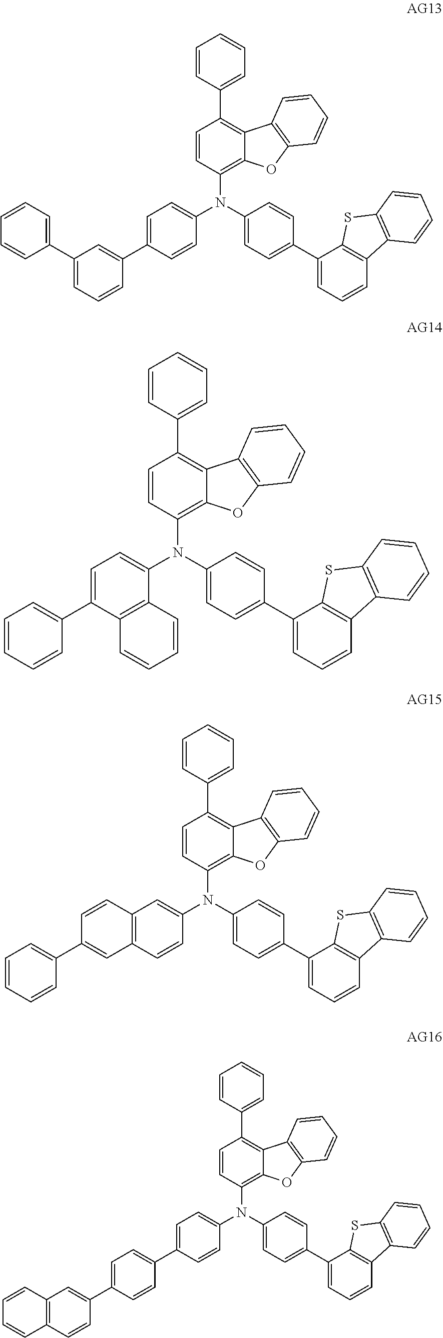 [SMILES: c1ccc(-c2cccc(-c3ccc(N(c4ccc(-c5cccc6c5sc5ccccc56)cc4)c4ccc(-c5ccccc5)c5c4oc4ccccc45)cc3)c2)cc1 ; c1ccc(-c2ccc(N(c3ccc(-c4cccc5c4sc4ccccc45)cc3)c3ccc(-c4ccccc4)c4c3oc3ccccc34)c3ccccc23)cc1 ; c1ccc(-c2ccc3cc(N(c4ccc(-c5cccc6c5sc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4cccc5c4sc4ccccc45)cc3)c3oc4ccccc4c23)cc1]

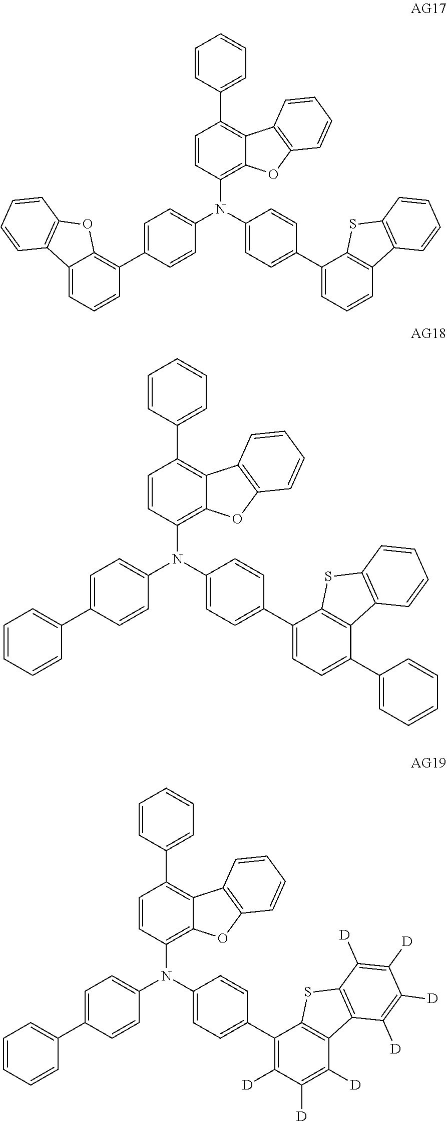 [SMILES: c1ccc(-c2ccc(N(c3ccc(-c4cccc5c4oc4ccccc45)cc3)c3ccc(-c4cccc5c4sc4ccccc45)cc3)c3oc4ccccc4c23)cc1 ; c1ccc(-c2ccc(N(c3ccc(-c4ccc(-c5ccccc5)c5c4sc4ccccc45)cc3)c3ccc(-c4ccccc4)c4c3oc3ccccc34)cc2)cc1 ; [2H]c1c([2H])c([2H])c2c(sc3c(-c4ccc(N(c5ccc(-c6ccccc6)cc5)c5ccc(-c6ccccc6)c6c5oc5ccccc56)cc4)c([2H])c([2H])c([2H])c32)c1[2H]]

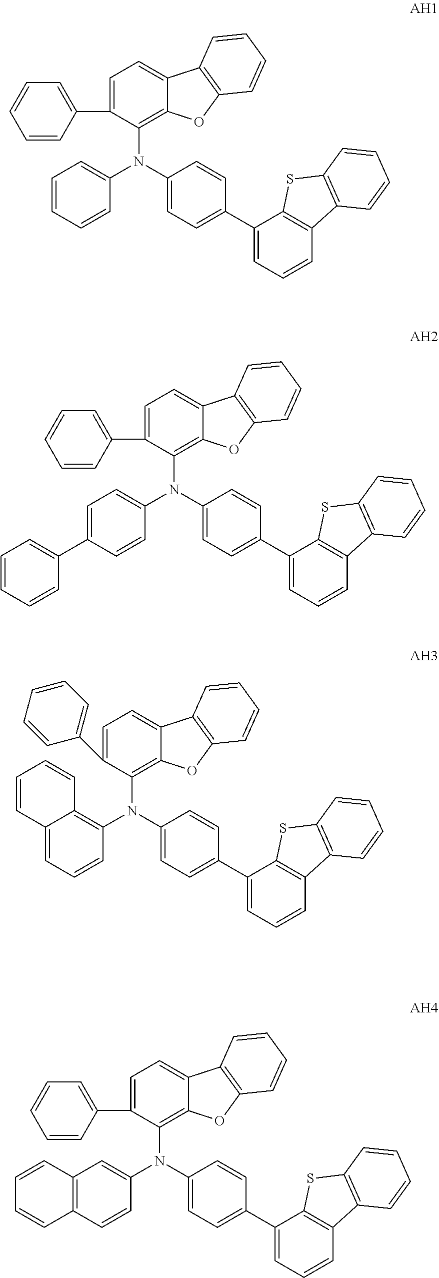 [SMILES: c1ccc(-c2ccc3c(oc4ccccc43)c2N(c2ccccc2)c2ccc(-c3cccc4c3sc3ccccc34)cc2)cc1 ; c1ccc(-c2ccc(N(c3ccc(-c4cccc5c4sc4ccccc45)cc3)c3c(-c4ccccc4)ccc4c3oc3ccccc34)cc2)cc1 ; c1ccc(-c2ccc3c(oc4ccccc43)c2N(c2ccc(-c3cccc4c3sc3ccccc34)cc2)c2cccc3ccccc23)cc1 ; c1ccc(-c2ccc3c(oc4ccccc43)c2N(c2ccc(-c3cccc4c3sc3ccccc34)cc2)c2ccc3ccccc3c2)cc1]

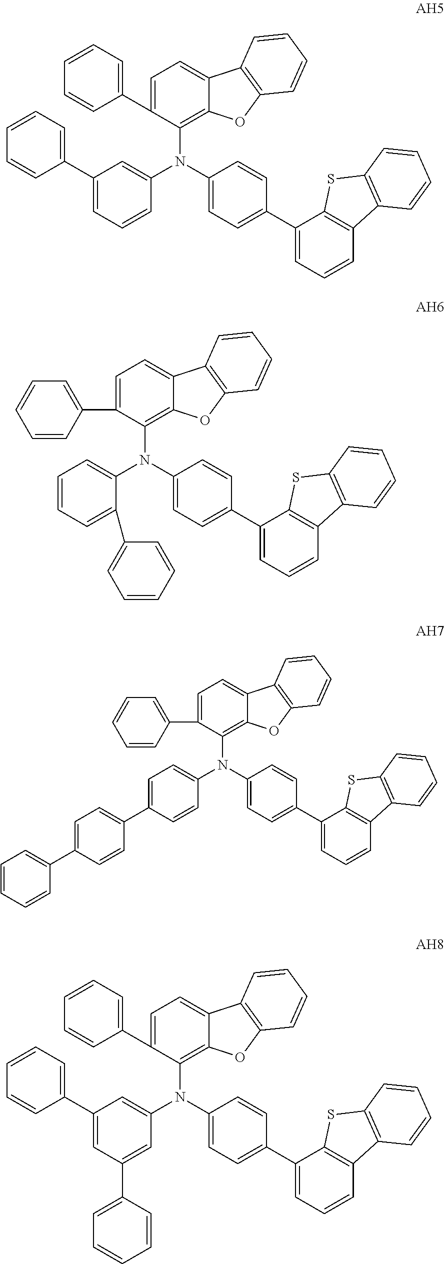 [SMILES: c1ccc(-c2cccc(N(c3ccc(-c4cccc5c4sc4ccccc45)cc3)c3c(-c4ccccc4)ccc4c3oc3ccccc34)c2)cc1 ; c1ccc(-c2ccccc2N(c2ccc(-c3cccc4c3sc3ccccc34)cc2)c2c(-c3ccccc3)ccc3c2oc2ccccc23)cc1 ; c1ccc(-c2ccc(-c3ccc(N(c4ccc(-c5cccc6c5sc5ccccc56)cc4)c4c(-c5ccccc5)ccc5c4oc4ccccc45)cc3)cc2)cc1 ; c1ccc(-c2cc(-c3ccccc3)cc(N(c3ccc(-c4cccc5c4sc4ccccc45)cc3)c3c(-c4ccccc4)ccc4c3oc3ccccc34)c2)cc1]

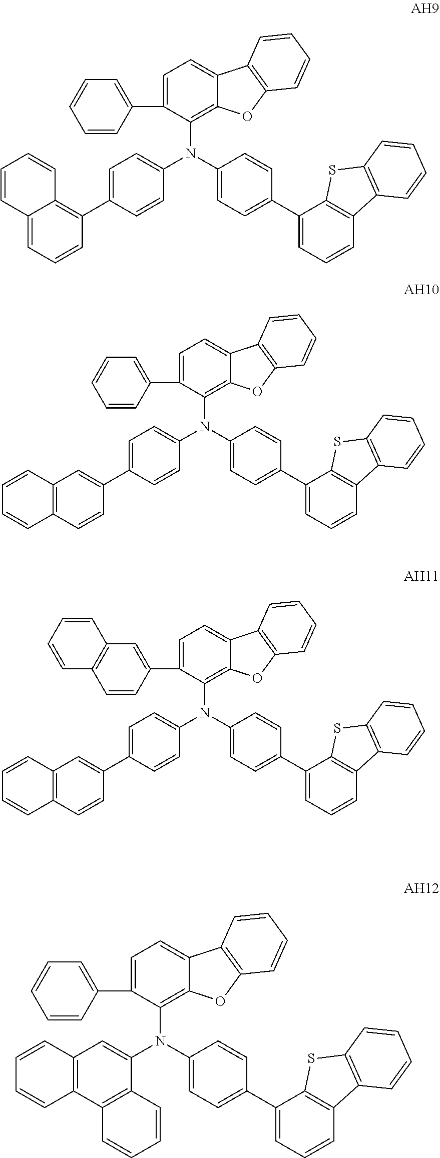 [SMILES: c1ccc(-c2ccc3c(oc4ccccc43)c2N(c2ccc(-c3cccc4ccccc34)cc2)c2ccc(-c3cccc4c3sc3ccccc34)cc2)cc1 ; c1ccc(-c2ccc3c(oc4ccccc43)c2N(c2ccc(-c3ccc4ccccc4c3)cc2)c2ccc(-c3cccc4c3sc3ccccc34)cc2)cc1 ; c1ccc2cc(-c3ccc(N(c4ccc(-c5cccc6c5sc5ccccc56)cc4)c4c(-c5ccc6ccccc6c5)ccc5c4oc4ccccc45)cc3)ccc2c1 ; c1ccc(-c2ccc3c(oc4ccccc43)c2N(c2ccc(-c3cccc4c3sc3ccccc34)cc2)c2cc3ccccc3c3ccccc23)cc1]

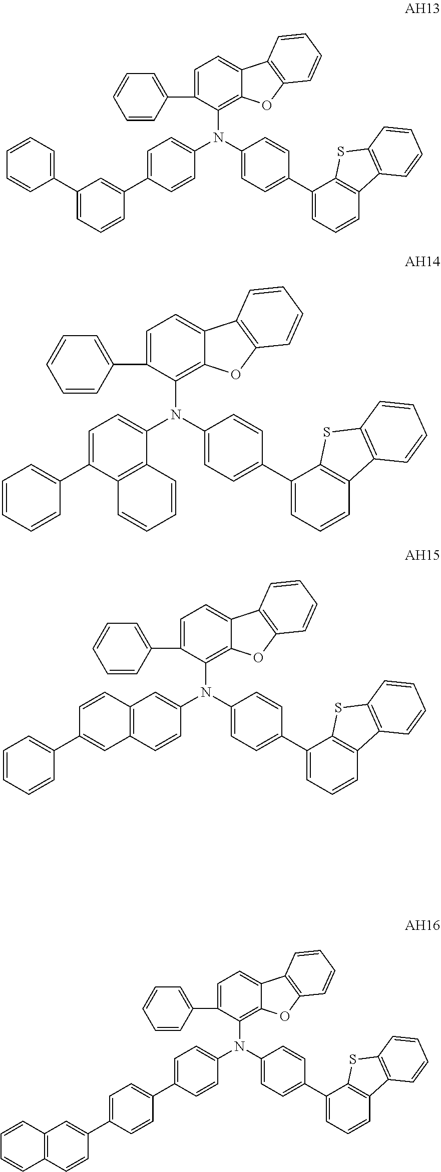 [SMILES: c1ccc(-c2cccc(-c3ccc(N(c4ccc(-c5cccc6c5sc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3cccc4c3sc3ccccc34)cc2)c2ccc(-c3ccccc3)c3ccccc23)cc1 ; c1ccc(-c2ccc3cc(N(c4ccc(-c5cccc6c5sc5ccccc56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4c3sc3ccccc34)cc2)cc1]

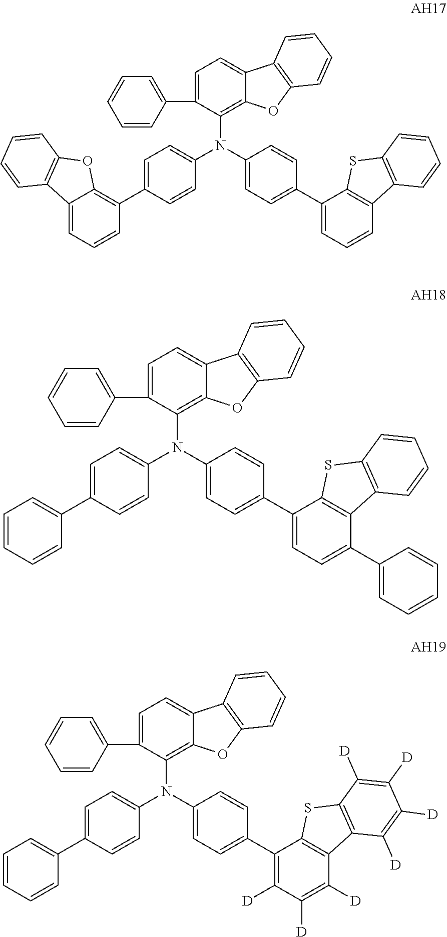 [SMILES: c1ccc(-c2ccc3c(oc4ccccc43)c2N(c2ccc(-c3cccc4c3oc3ccccc34)cc2)c2ccc(-c3cccc4c3sc3ccccc34)cc2)cc1 ; c1ccc(-c2ccc(N(c3ccc(-c4ccc(-c5ccccc5)c5c4sc4ccccc45)cc3)c3c(-c4ccccc4)ccc4c3oc3ccccc34)cc2)cc1 ; [2H]c1c([2H])c([2H])c2c(sc3c(-c4ccc(N(c5ccc(-c6ccccc6)cc5)c5c(-c6ccccc6)ccc6c5oc5ccccc56)cc4)c([2H])c([2H])c([2H])c32)c1[2H]]

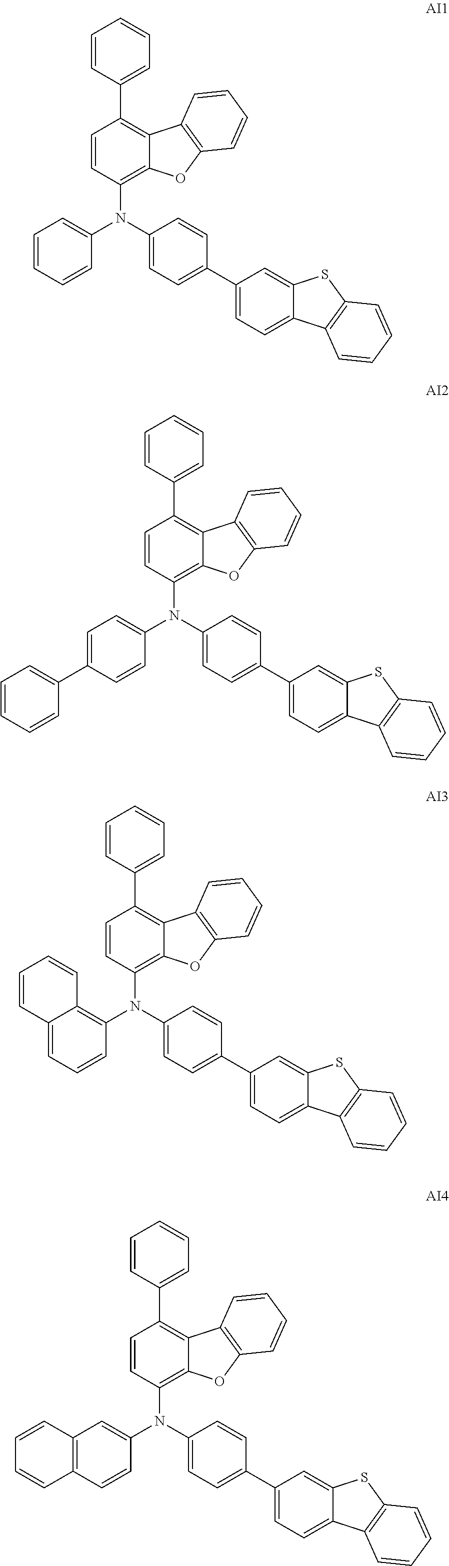 [SMILES: c1ccc(-c2ccc(N(c3ccccc3)c3ccc(-c4ccc5c(c4)sc4ccccc45)cc3)c3oc4ccccc4c23)cc1 ; c1ccc(-c2ccc(N(c3ccc(-c4ccc5c(c4)sc4ccccc45)cc3)c3ccc(-c4ccccc4)c4c3oc3ccccc34)cc2)cc1 ; c1ccc(-c2ccc(N(c3ccc(-c4ccc5c(c4)sc4ccccc45)cc3)c3cccc4ccccc34)c3oc4ccccc4c23)cc1 ; c1ccc(-c2ccc(N(c3ccc(-c4ccc5c(c4)sc4ccccc45)cc3)c3ccc4ccccc4c3)c3oc4ccccc4c23)cc1]

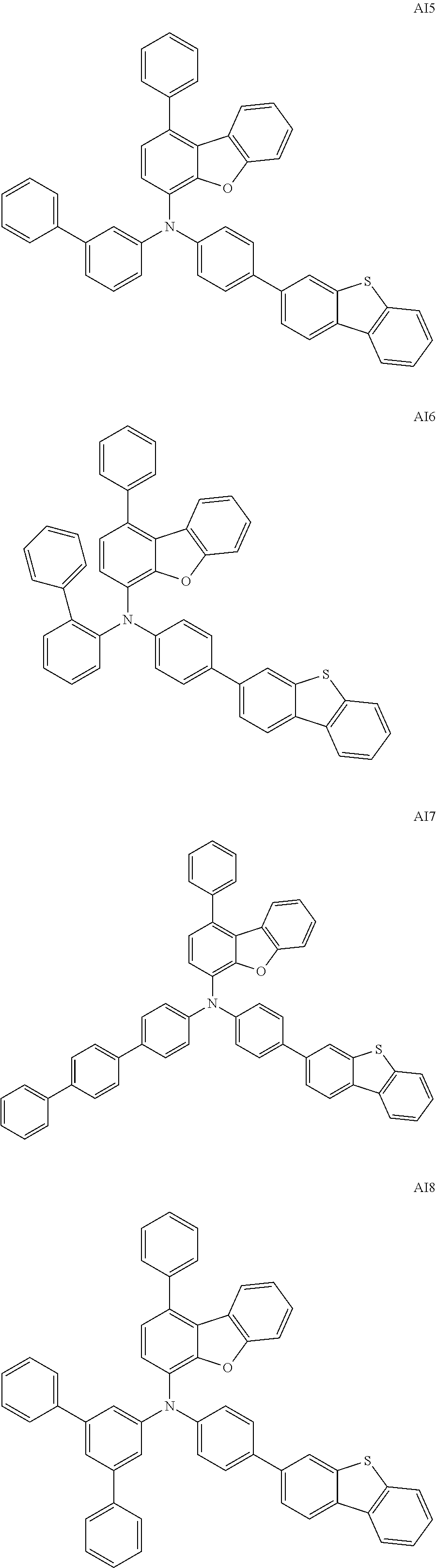 [SMILES: c1ccc(-c2cccc(N(c3ccc(-c4ccc5c(c4)sc4ccccc45)cc3)c3ccc(-c4ccccc4)c4c3oc3ccccc34)c2)cc1 ; c1ccc(-c2ccccc2N(c2ccc(-c3ccc4c(c3)sc3ccccc34)cc2)c2ccc(-c3ccccc3)c3c2oc2ccccc23)cc1 ; c1ccc(-c2ccc(-c3ccc(N(c4ccc(-c5ccc6c(c5)sc5ccccc56)cc4)c4ccc(-c5ccccc5)c5c4oc4ccccc45)cc3)cc2)cc1 ; c1ccc(-c2cc(-c3ccccc3)cc(N(c3ccc(-c4ccc5c(c4)sc4ccccc45)cc3)c3ccc(-c4ccccc4)c4c3oc3ccccc34)c2)cc1]

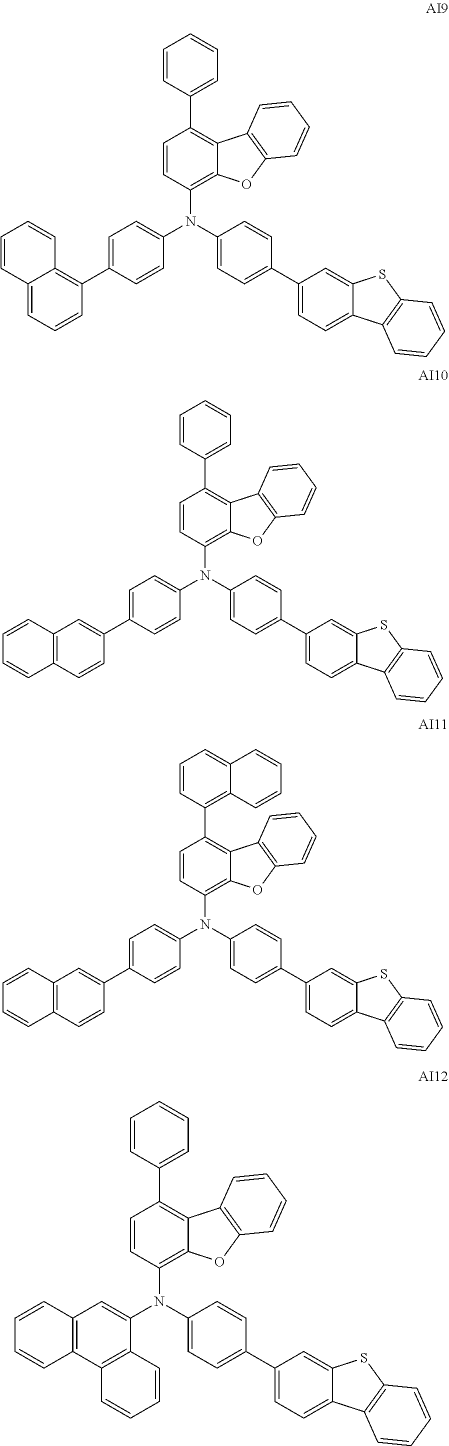 [SMILES: c1ccc(-c2ccc(N(c3ccc(-c4ccc5c(c4)sc4ccccc45)cc3)c3ccc(-c4cccc5ccccc45)cc3)c3oc4ccccc4c23)cc1 ; c1ccc(-c2ccc(N(c3ccc(-c4ccc5ccccc5c4)cc3)c3ccc(-c4ccc5c(c4)sc4ccccc45)cc3)c3oc4ccccc4c23)cc1 ; c1ccc2cc(-c3ccc(N(c4ccc(-c5ccc6c(c5)sc5ccccc56)cc4)c4ccc(-c5cccc6ccccc56)c5c4oc4ccccc45)cc3)ccc2c1 ; c1ccc(-c2ccc(N(c3ccc(-c4ccc5c(c4)sc4ccccc45)cc3)c3cc4ccccc4c4ccccc34)c3oc4ccccc4c23)cc1]

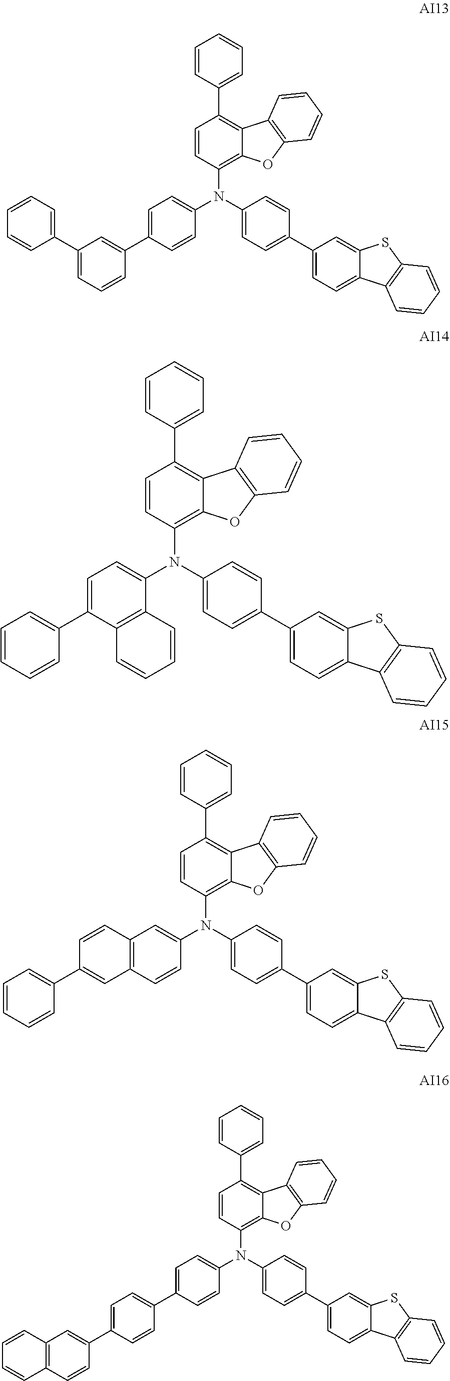 [SMILES: c1ccc(-c2cccc(-c3ccc(N(c4ccc(-c5ccc6c(c5)sc5ccccc56)cc4)c4ccc(-c5ccccc5)c5c4oc4ccccc45)cc3)c2)cc1 ; c1ccc(-c2ccc(N(c3ccc(-c4ccc5c(c4)sc4ccccc45)cc3)c3ccc(-c4ccccc4)c4c3oc3ccccc34)c3ccccc23)cc1 ; c1ccc(-c2ccc3cc(N(c4ccc(-c5ccc6c(c5)sc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4ccc5c(c4)sc4ccccc45)cc3)c3oc4ccccc4c23)cc1]

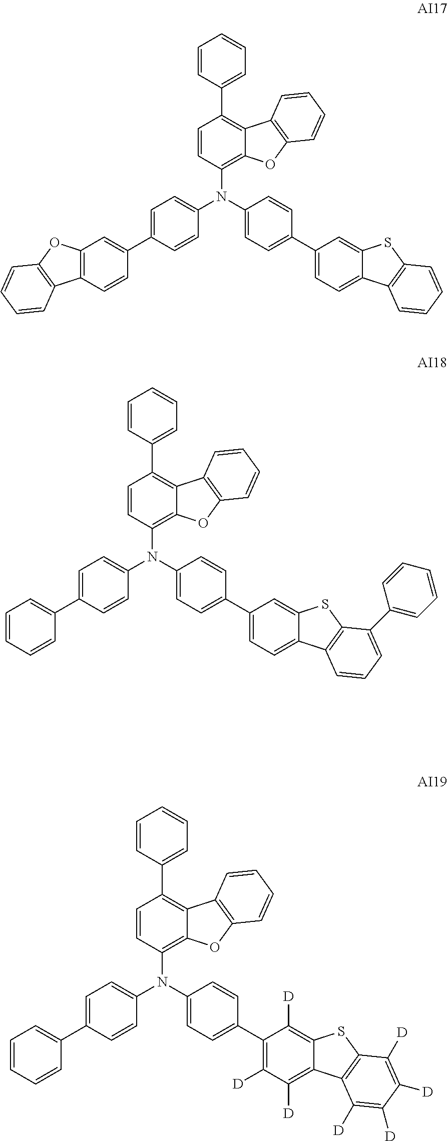 [SMILES: c1ccc(-c2ccc(N(c3ccc(-c4ccc5c(c4)oc4ccccc45)cc3)c3ccc(-c4ccc5c(c4)sc4ccccc45)cc3)c3oc4ccccc4c23)cc1 ; c1ccc(-c2ccc(N(c3ccc(-c4ccc5c(c4)sc4c(-c6ccccc6)cccc45)cc3)c3ccc(-c4ccccc4)c4c3oc3ccccc34)cc2)cc1 ; [2H]c1c([2H])c([2H])c2c(sc3c([2H])c(-c4ccc(N(c5ccc(-c6ccccc6)cc5)c5ccc(-c6ccccc6)c6c5oc5ccccc56)cc4)c([2H])c([2H])c32)c1[2H]]

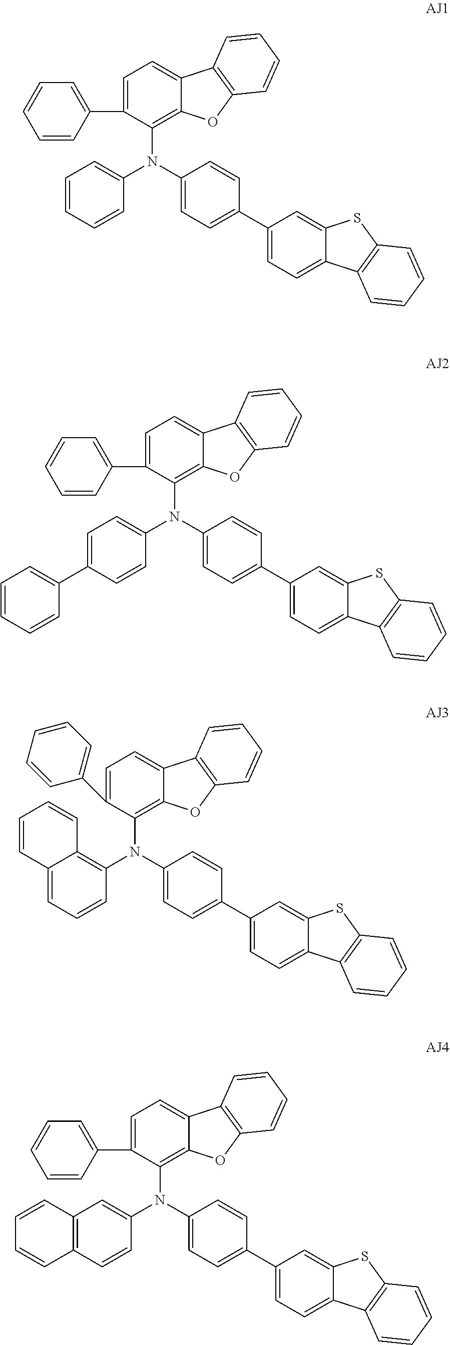 [SMILES: c1ccc(-c2ccc3c(oc4ccccc43)c2N(c2ccccc2)c2ccc(-c3ccc4c(c3)sc3ccccc34)cc2)cc1 ; c1ccc(-c2ccc(N(c3ccc(-c4ccc5c(c4)sc4ccccc45)cc3)c3c(-c4ccccc4)ccc4c3oc3ccccc34)cc2)cc1 ; c1ccc(-c2ccc3c(oc4ccccc43)c2N(c2ccc(-c3ccc4c(c3)sc3ccccc34)cc2)c2cccc3ccccc23)cc1 ; c1ccc(-c2ccc3c(oc4ccccc43)c2N(c2ccc(-c3ccc4c(c3)sc3ccccc34)cc2)c2ccc3ccccc3c2)cc1]

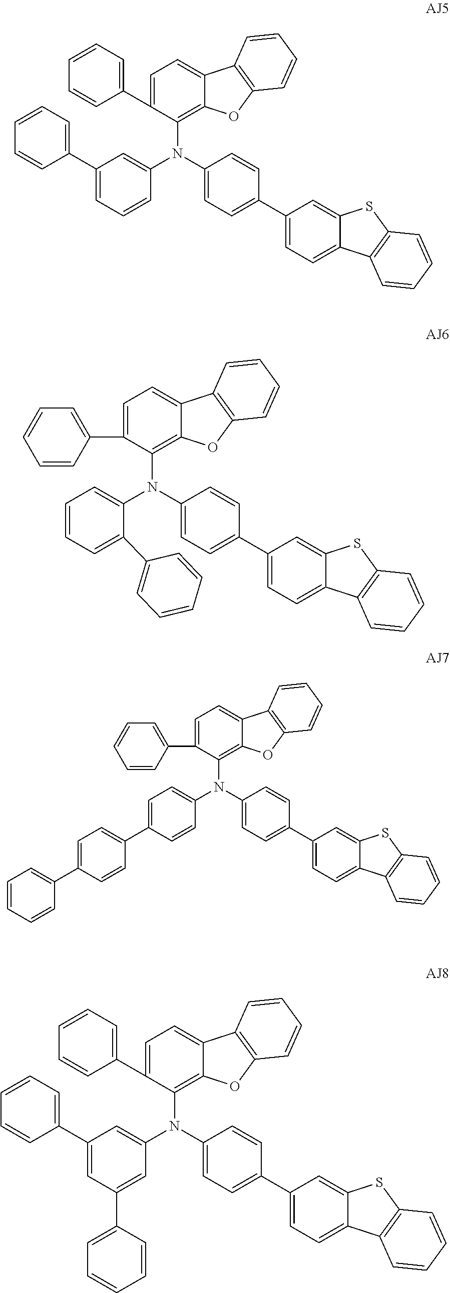 [SMILES: c1ccc(-c2cccc(N(c3ccc(-c4ccc5c(c4)sc4ccccc45)cc3)c3c(-c4ccccc4)ccc4c3oc3ccccc34)c2)cc1 ; c1ccc(-c2ccccc2N(c2ccc(-c3ccc4c(c3)sc3ccccc34)cc2)c2c(-c3ccccc3)ccc3c2oc2ccccc23)cc1 ; c1ccc(-c2ccc(-c3ccc(N(c4ccc(-c5ccc6c(c5)sc5ccccc56)cc4)c4c(-c5ccccc5)ccc5c4oc4ccccc45)cc3)cc2)cc1 ; c1ccc(-c2cc(-c3ccccc3)cc(N(c3ccc(-c4ccc5c(c4)sc4ccccc45)cc3)c3c(-c4ccccc4)ccc4c3oc3ccccc34)c2)cc1]

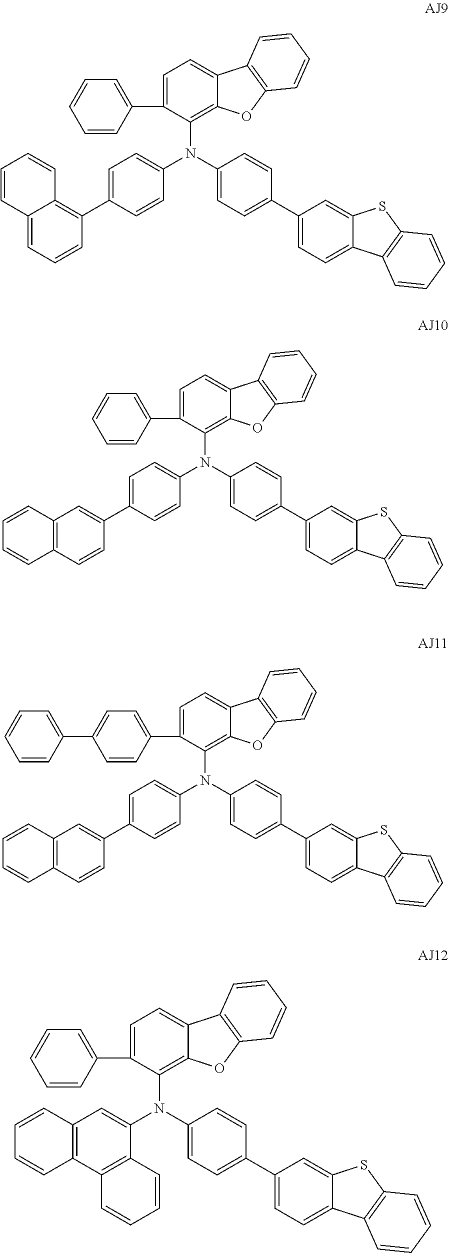 [SMILES: c1ccc(-c2ccc3c(oc4ccccc43)c2N(c2ccc(-c3ccc4c(c3)sc3ccccc34)cc2)c2ccc(-c3cccc4ccccc34)cc2)cc1 ; c1ccc(-c2ccc3c(oc4ccccc43)c2N(c2ccc(-c3ccc4ccccc4c3)cc2)c2ccc(-c3ccc4c(c3)sc3ccccc34)cc2)cc1 ; c1ccc(-c2ccc(-c3ccc4c(oc5ccccc54)c3N(c3ccc(-c4ccc5ccccc5c4)cc3)c3ccc(-c4ccc5c(c4)sc4ccccc45)cc3)cc2)cc1 ; c1ccc(-c2ccc3c(oc4ccccc43)c2N(c2ccc(-c3ccc4c(c3)sc3ccccc34)cc2)c2cc3ccccc3c3ccccc23)cc1]

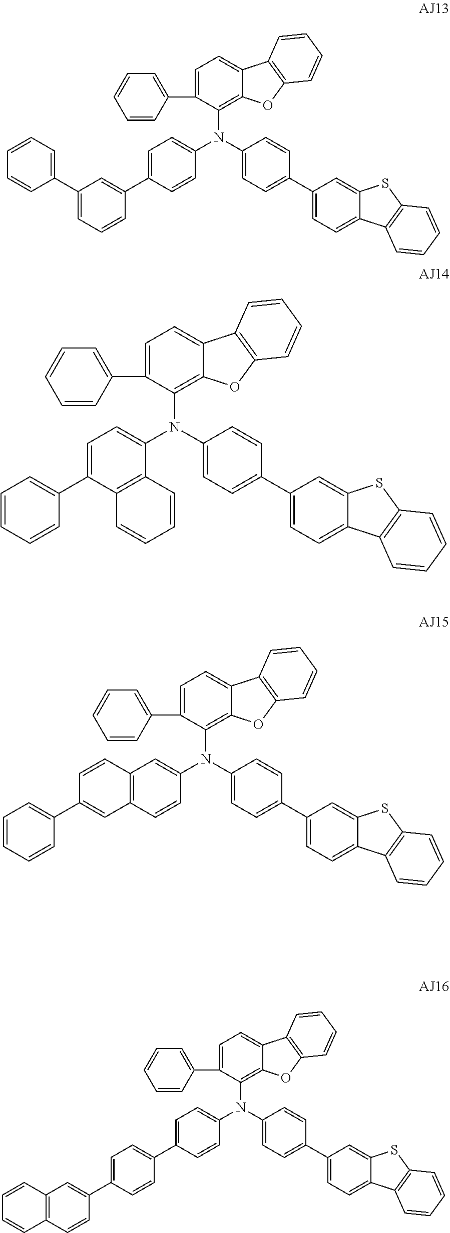 [SMILES: c1ccc(-c2cccc(-c3ccc(N(c4ccc(-c5ccc6c(c5)sc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3ccc4c(c3)sc3ccccc34)cc2)c2ccc(-c3ccccc3)c3ccccc23)cc1 ; c1ccc(-c2ccc3cc(N(c4ccc(-c5ccc6c(c5)sc5ccccc56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3ccc4c(c3)sc3ccccc34)cc2)cc1]

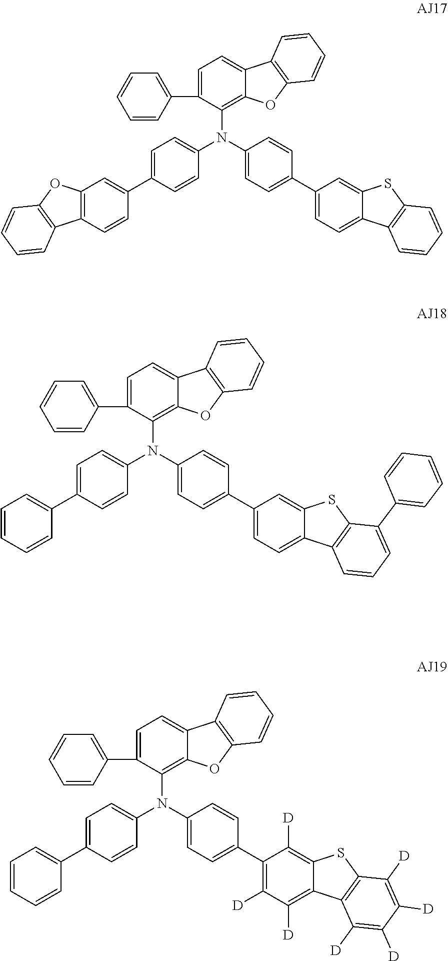 [SMILES: c1ccc(-c2ccc3c(oc4ccccc43)c2N(c2ccc(-c3ccc4c(c3)oc3ccccc34)cc2)c2ccc(-c3ccc4c(c3)sc3ccccc34)cc2)cc1 ; c1ccc(-c2ccc(N(c3ccc(-c4ccc5c(c4)sc4c(-c6ccccc6)cccc45)cc3)c3c(-c4ccccc4)ccc4c3oc3ccccc34)cc2)cc1 ; [2H]c1c([2H])c([2H])c2c(sc3c([2H])c(-c4ccc(N(c5ccc(-c6ccccc6)cc5)c5c(-c6ccccc6)ccc6c5oc5ccccc56)cc4)c([2H])c([2H])c32)c1[2H]]

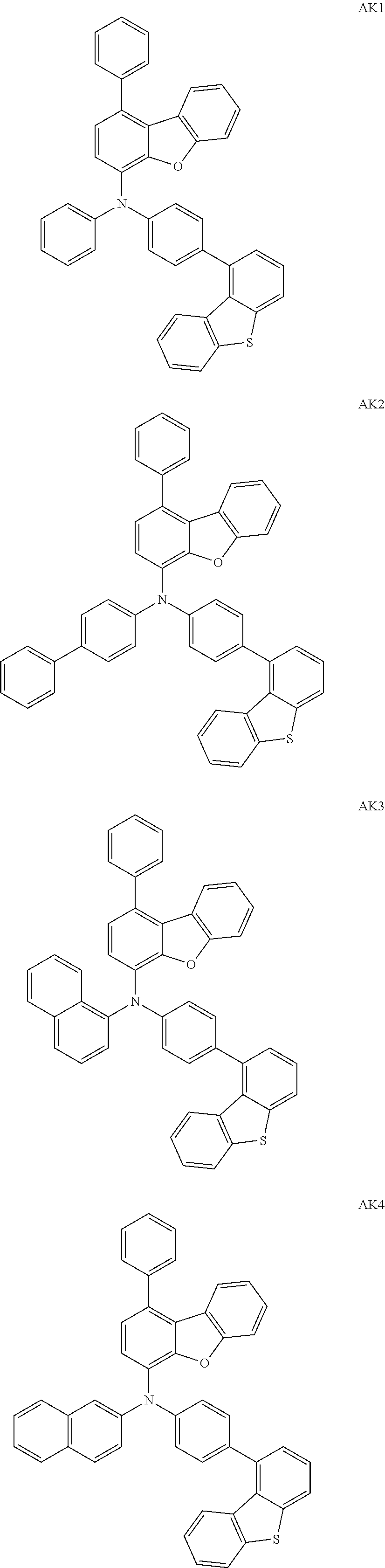 [SMILES: c1ccc(-c2ccc(N(c3ccccc3)c3ccc(-c4cccc5sc6ccccc6c45)cc3)c3oc4ccccc4c23)cc1 ; c1ccc(-c2ccc(N(c3ccc(-c4cccc5sc6ccccc6c45)cc3)c3ccc(-c4ccccc4)c4c3oc3ccccc34)cc2)cc1 ; c1ccc(-c2ccc(N(c3ccc(-c4cccc5sc6ccccc6c45)cc3)c3cccc4ccccc34)c3oc4ccccc4c23)cc1 ; c1ccc(-c2ccc(N(c3ccc(-c4cccc5sc6ccccc6c45)cc3)c3ccc4ccccc4c3)c3oc4ccccc4c23)cc1]

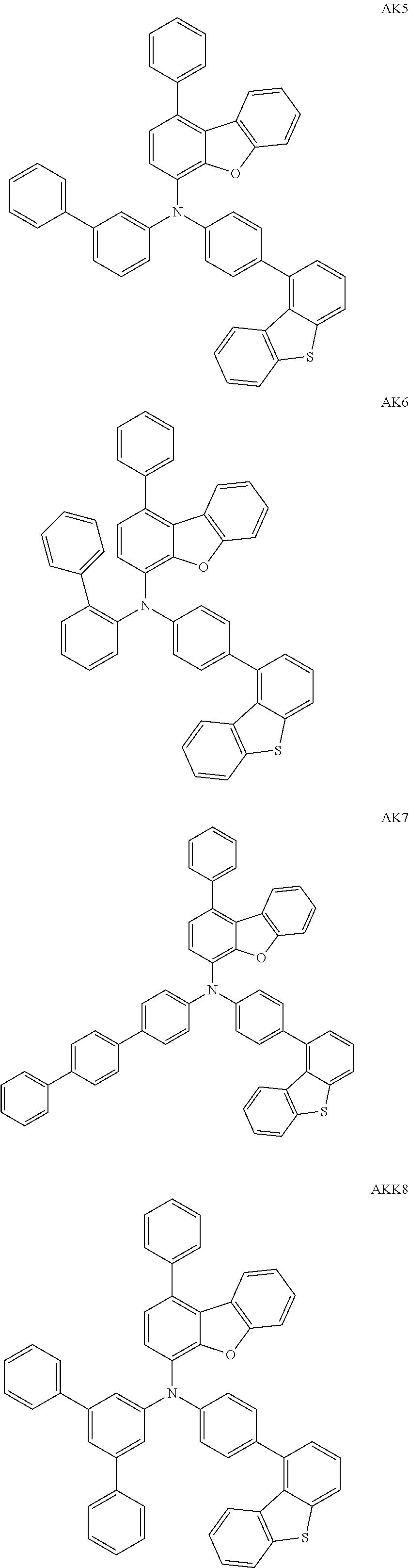 [SMILES: c1ccc(-c2cccc(N(c3ccc(-c4cccc5sc6ccccc6c45)cc3)c3ccc(-c4ccccc4)c4c3oc3ccccc34)c2)cc1 ; c1ccc(-c2ccccc2N(c2ccc(-c3cccc4sc5ccccc5c34)cc2)c2ccc(-c3ccccc3)c3c2oc2ccccc23)cc1 ; c1ccc(-c2ccc(-c3ccc(N(c4ccc(-c5cccc6sc7ccccc7c56)cc4)c4ccc(-c5ccccc5)c5c4oc4ccccc45)cc3)cc2)cc1 ; c1ccc(-c2cc(-c3ccccc3)cc(N(c3ccc(-c4cccc5sc6ccccc6c45)cc3)c3ccc(-c4ccccc4)c4c3oc3ccccc34)c2)cc1]

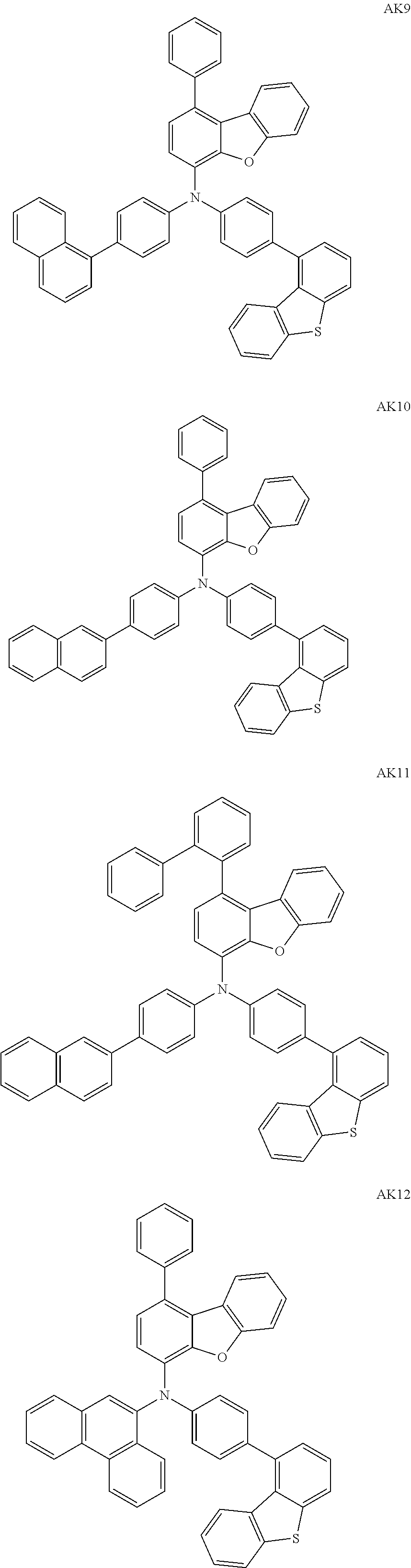 [SMILES: c1ccc(-c2ccc(N(c3ccc(-c4cccc5ccccc45)cc3)c3ccc(-c4cccc5sc6ccccc6c45)cc3)c3oc4ccccc4c23)cc1 ; c1ccc(-c2ccc(N(c3ccc(-c4ccc5ccccc5c4)cc3)c3ccc(-c4cccc5sc6ccccc6c45)cc3)c3oc4ccccc4c23)cc1 ; c1ccc(-c2ccccc2-c2ccc(N(c3ccc(-c4ccc5ccccc5c4)cc3)c3ccc(-c4cccc5sc6ccccc6c45)cc3)c3oc4ccccc4c23)cc1 ; c1ccc(-c2ccc(N(c3ccc(-c4cccc5sc6ccccc6c45)cc3)c3cc4ccccc4c4ccccc34)c3oc4ccccc4c23)cc1]

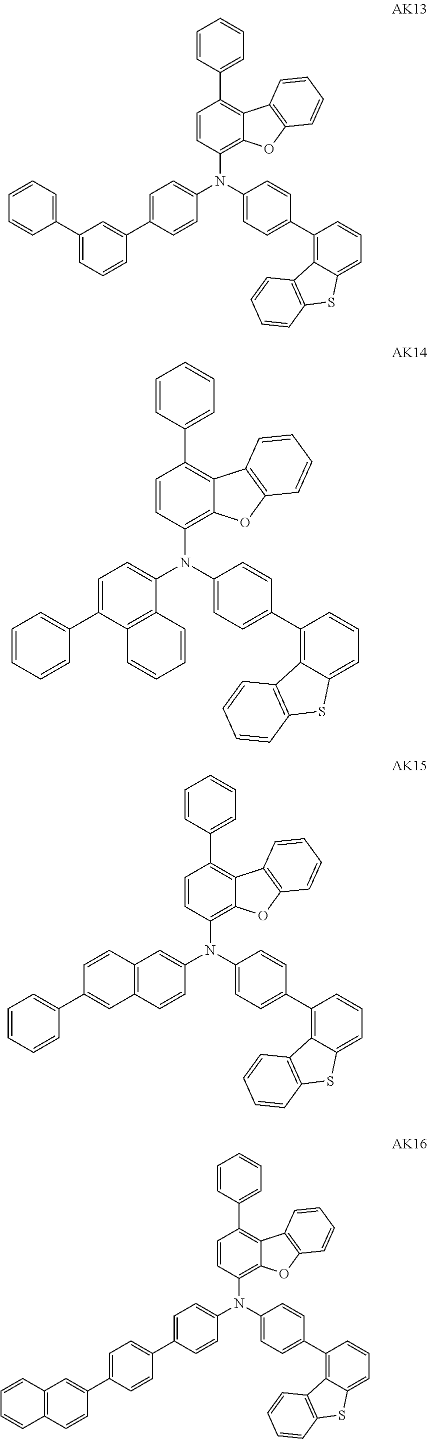 [SMILES: c1ccc(-c2cccc(-c3ccc(N(c4ccc(-c5cccc6sc7ccccc7c56)cc4)c4ccc(-c5ccccc5)c5c4oc4ccccc45)cc3)c2)cc1 ; c1ccc(-c2ccc(N(c3ccc(-c4cccc5sc6ccccc6c45)cc3)c3ccc(-c4ccccc4)c4c3oc3ccccc34)c3ccccc23)cc1 ; c1ccc(-c2ccc3cc(N(c4ccc(-c5cccc6sc7ccccc7c56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4cccc5sc6ccccc6c45)cc3)c3oc4ccccc4c23)cc1]

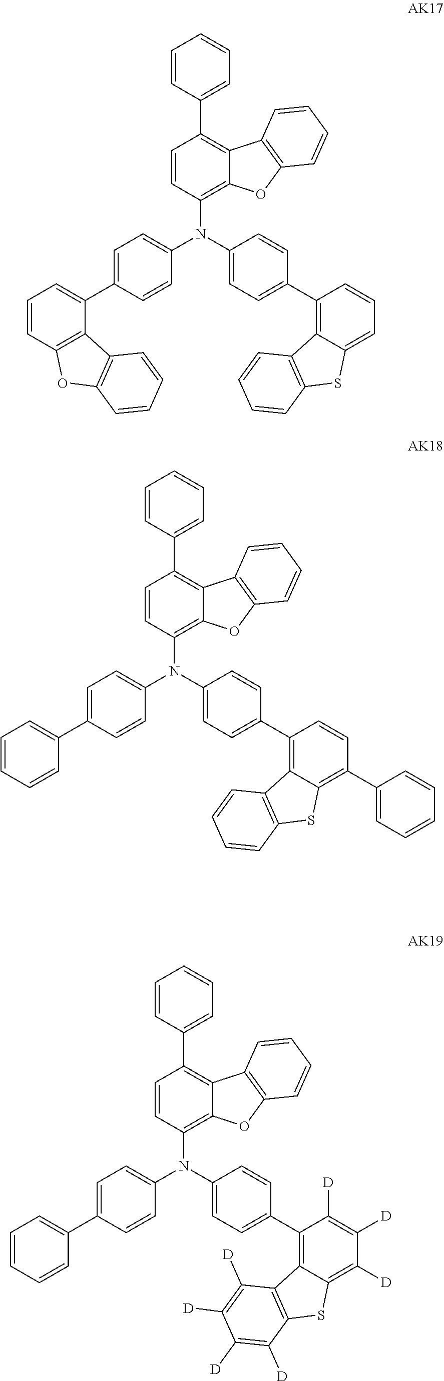 [SMILES: c1ccc(-c2ccc(N(c3ccc(-c4cccc5oc6ccccc6c45)cc3)c3ccc(-c4cccc5sc6ccccc6c45)cc3)c3oc4ccccc4c23)cc1 ; c1ccc(-c2ccc(N(c3ccc(-c4ccc(-c5ccccc5)c5sc6ccccc6c45)cc3)c3ccc(-c4ccccc4)c4c3oc3ccccc34)cc2)cc1 ; [2H]c1c([2H])c([2H])c2c(sc3c([2H])c([2H])c([2H])c(-c4ccc(N(c5ccc(-c6ccccc6)cc5)c5ccc(-c6ccccc6)c6c5oc5ccccc56)cc4)c32)c1[2H]]

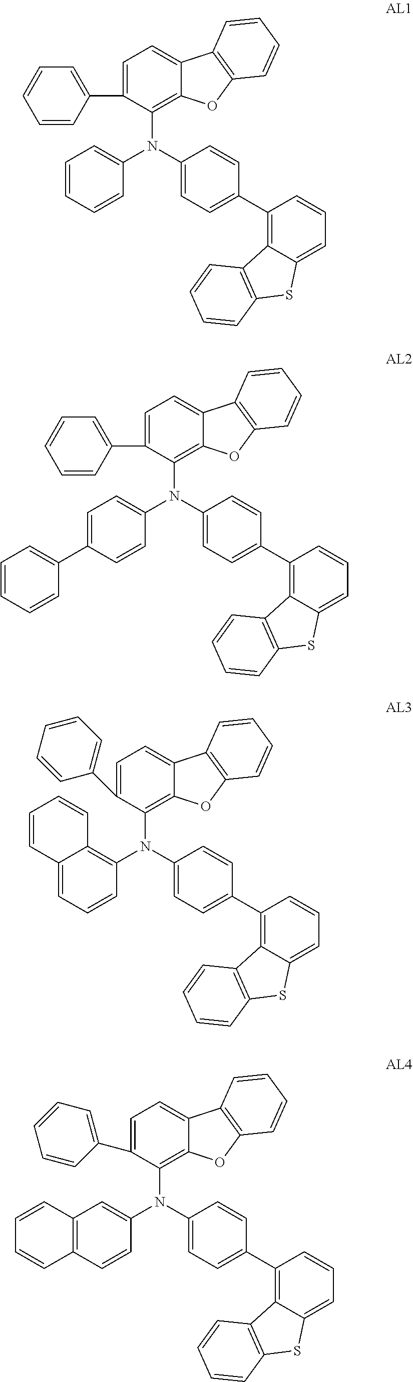

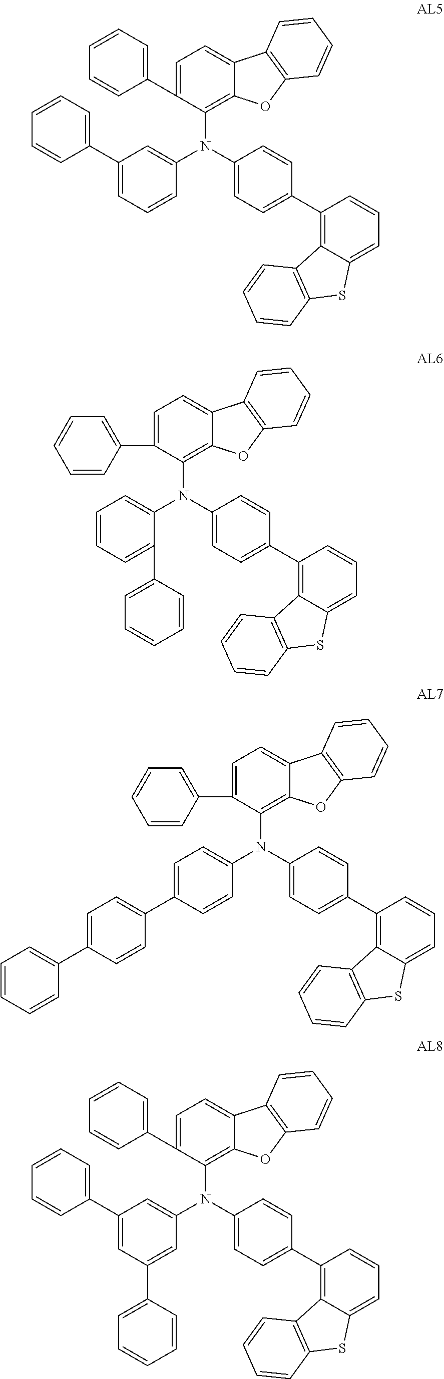

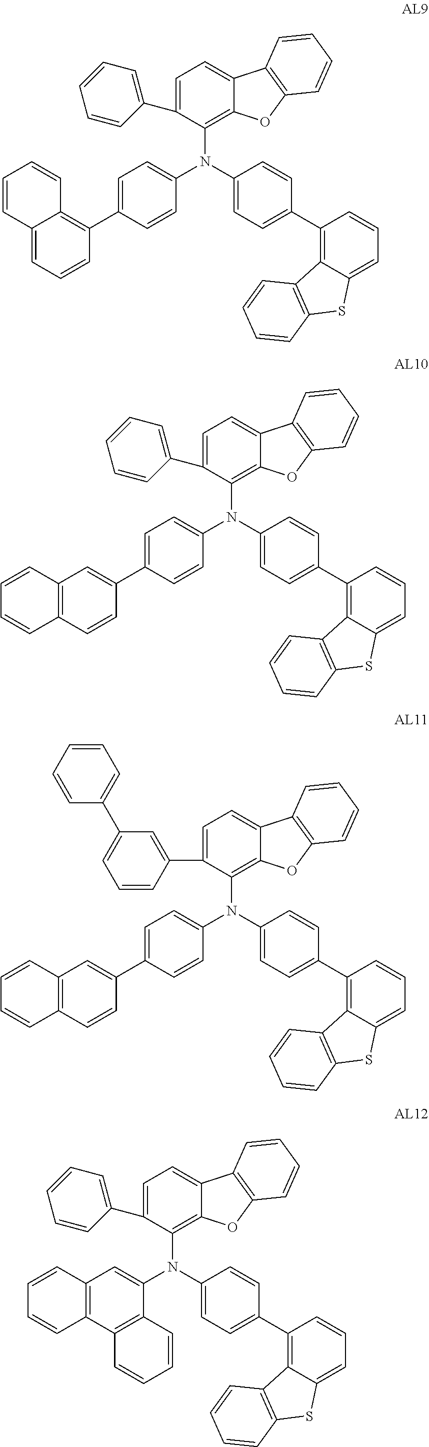 [SMILES: c1ccc(-c2ccc3c(oc4ccccc43)c2N(c2ccc(-c3cccc4ccccc34)cc2)c2ccc(-c3cccc4sc5ccccc5c34)cc2)cc1 ; c1ccc(-c2ccc3c(oc4ccccc43)c2N(c2ccc(-c3ccc4ccccc4c3)cc2)c2ccc(-c3cccc4sc5ccccc5c34)cc2)cc1 ; c1ccc(-c2cccc(-c3ccc4c(oc5ccccc54)c3N(c3ccc(-c4ccc5ccccc5c4)cc3)c3ccc(-c4cccc5sc6ccccc6c45)cc3)c2)cc1 ; c1ccc(-c2ccc3c(oc4ccccc43)c2N(c2ccc(-c3cccc4sc5ccccc5c34)cc2)c2cc3ccccc3c3ccccc23)cc1]

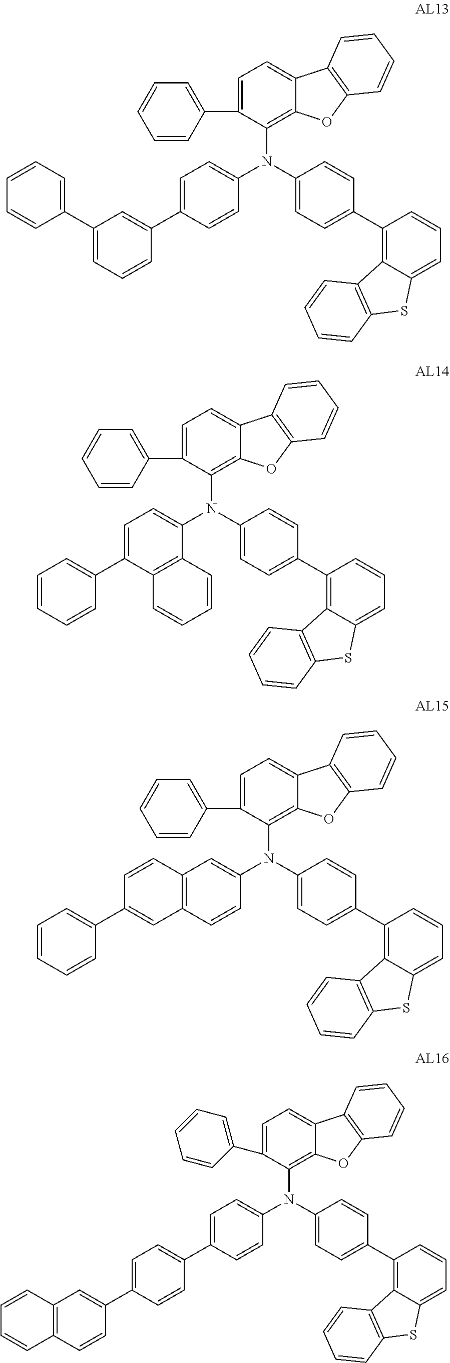

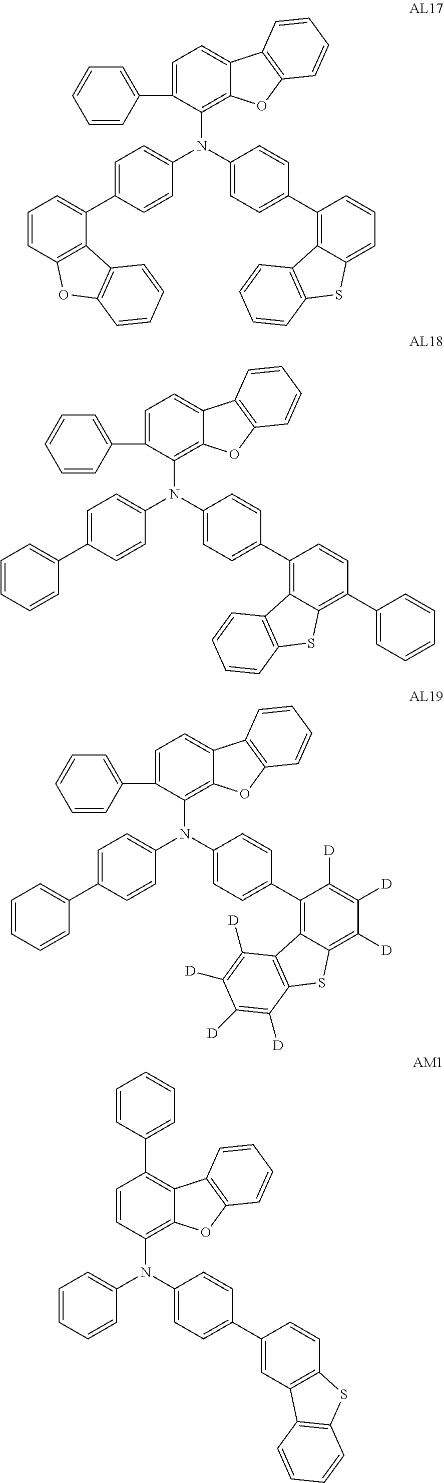

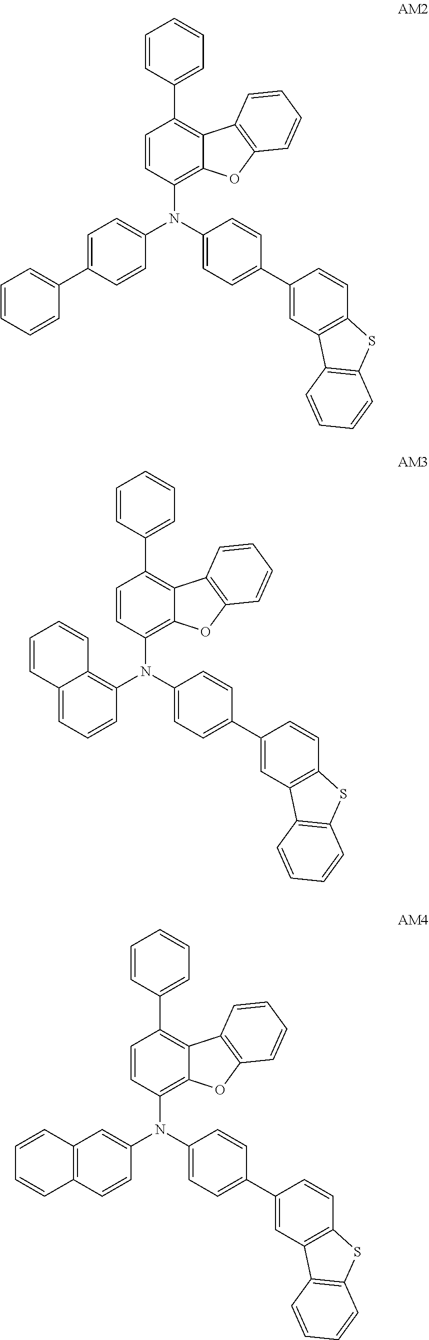 [SMILES: c1ccc(-c2ccc(N(c3ccc(-c4ccc5sc6ccccc6c5c4)cc3)c3ccc(-c4ccccc4)c4c3oc3ccccc34)cc2)cc1 ; c1ccc(-c2ccc(N(c3ccc(-c4ccc5sc6ccccc6c5c4)cc3)c3cccc4ccccc34)c3oc4ccccc4c23)cc1 ; c1ccc(-c2ccc(N(c3ccc(-c4ccc5sc6ccccc6c5c4)cc3)c3ccc4ccccc4c3)c3oc4ccccc4c23)cc1]

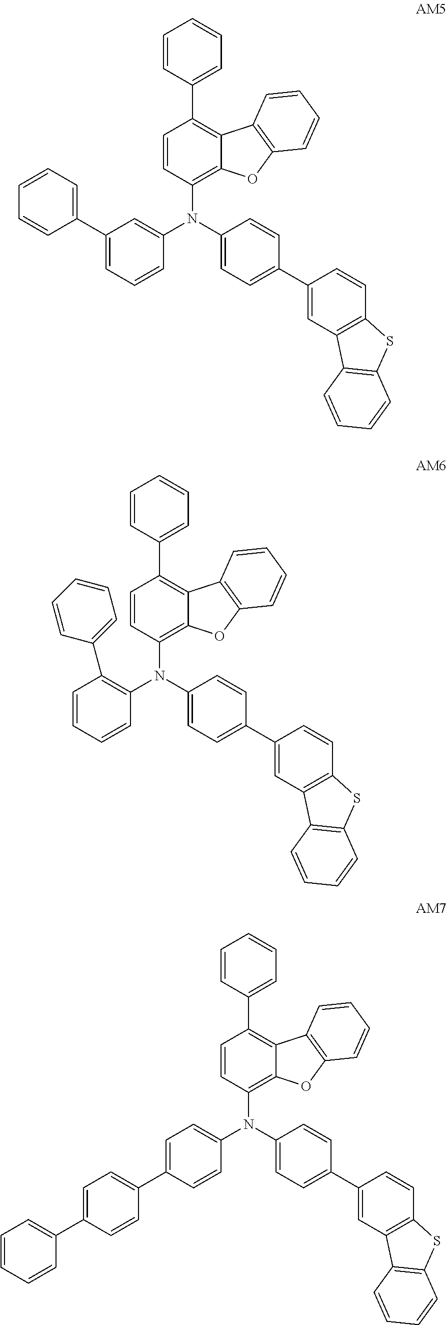 [SMILES: c1ccc(-c2cccc(N(c3ccc(-c4ccc5sc6ccccc6c5c4)cc3)c3ccc(-c4ccccc4)c4c3oc3ccccc34)c2)cc1 ; c1ccc(-c2ccccc2N(c2ccc(-c3ccc4sc5ccccc5c4c3)cc2)c2ccc(-c3ccccc3)c3c2oc2ccccc23)cc1 ; c1ccc(-c2ccc(-c3ccc(N(c4ccc(-c5ccc6sc7ccccc7c6c5)cc4)c4ccc(-c5ccccc5)c5c4oc4ccccc45)cc3)cc2)cc1]

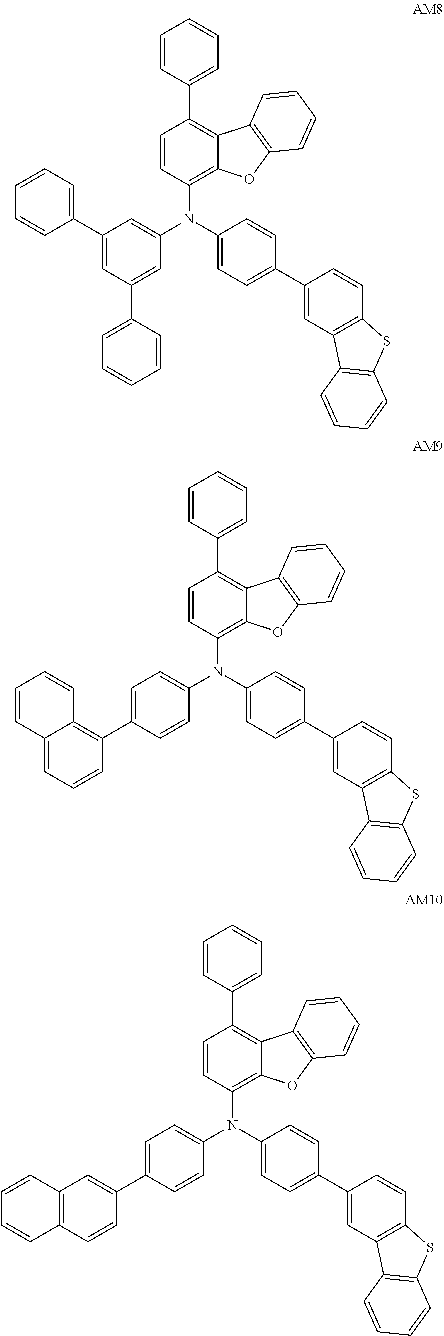 [SMILES: c1ccc(-c2cc(-c3ccccc3)cc(N(c3ccc(-c4ccc5sc6ccccc6c5c4)cc3)c3ccc(-c4ccccc4)c4c3oc3ccccc34)c2)cc1 ; c1ccc(-c2ccc(N(c3ccc(-c4ccc5sc6ccccc6c5c4)cc3)c3ccc(-c4cccc5ccccc45)cc3)c3oc4ccccc4c23)cc1 ; c1ccc(-c2ccc(N(c3ccc(-c4ccc5ccccc5c4)cc3)c3ccc(-c4ccc5sc6ccccc6c5c4)cc3)c3oc4ccccc4c23)cc1]

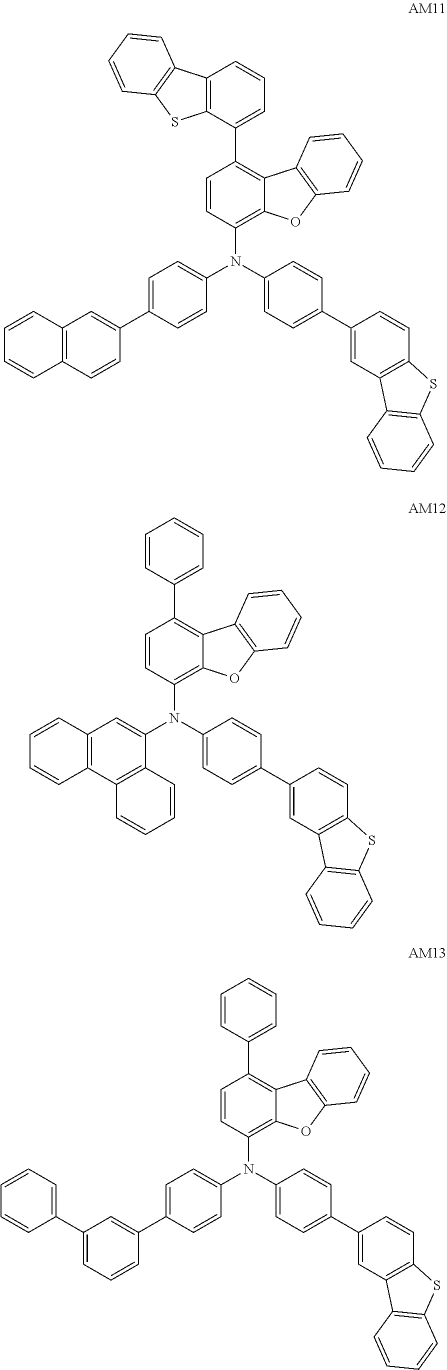 [SMILES: c1ccc2cc(-c3ccc(N(c4ccc(-c5ccc6sc7ccccc7c6c5)cc4)c4ccc(-c5cccc6c5sc5ccccc56)c5c4oc4ccccc45)cc3)ccc2c1 ; c1ccc(-c2ccc(N(c3ccc(-c4ccc5sc6ccccc6c5c4)cc3)c3cc4ccccc4c4ccccc34)c3oc4ccccc4c23)cc1 ; c1ccc(-c2cccc(-c3ccc(N(c4ccc(-c5ccc6sc7ccccc7c6c5)cc4)c4ccc(-c5ccccc5)c5c4oc4ccccc45)cc3)c2)cc1]

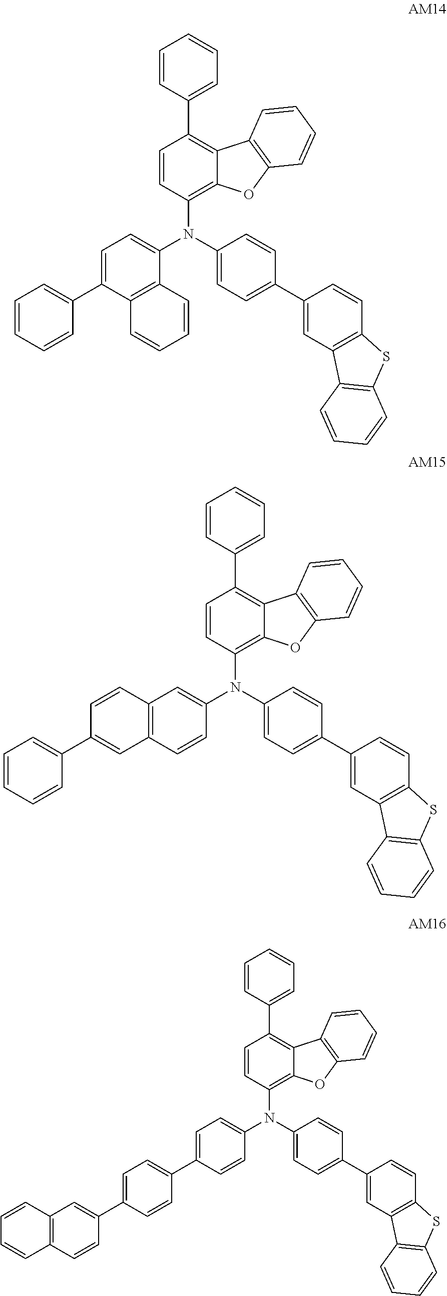 [SMILES: c1ccc(-c2ccc(N(c3ccc(-c4ccc5sc6ccccc6c5c4)cc3)c3ccc(-c4ccccc4)c4c3oc3ccccc34)c3ccccc23)cc1 ; c1ccc(-c2ccc3cc(N(c4ccc(-c5ccc6sc7ccccc7c6c5)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4ccc5sc6ccccc6c5c4)cc3)c3oc4ccccc4c23)cc1]

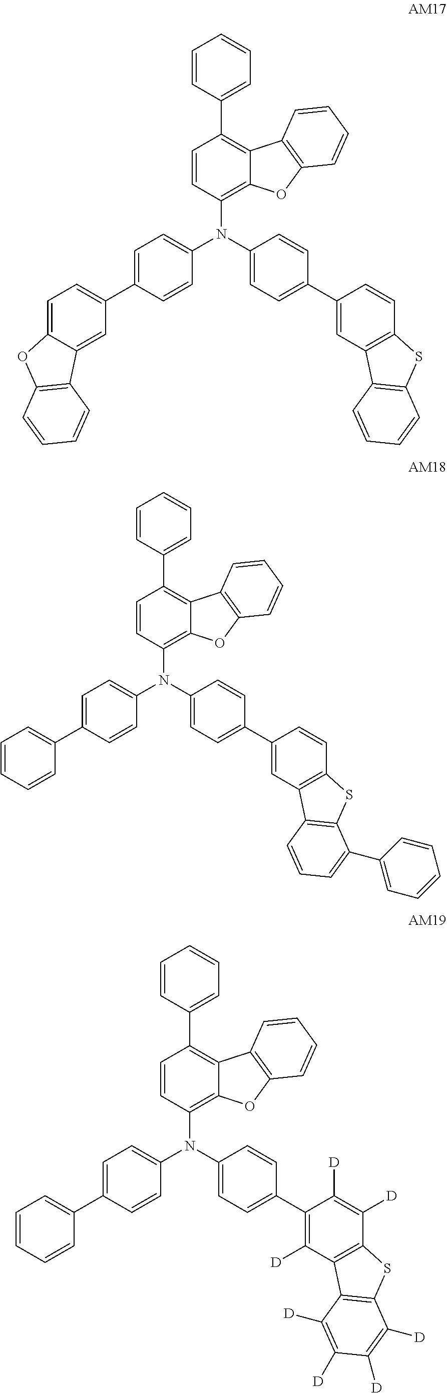 [SMILES: c1ccc(-c2ccc(N(c3ccc(-c4ccc5oc6ccccc6c5c4)cc3)c3ccc(-c4ccc5sc6ccccc6c5c4)cc3)c3oc4ccccc4c23)cc1 ; c1ccc(-c2ccc(N(c3ccc(-c4ccc5sc6c(-c7ccccc7)cccc6c5c4)cc3)c3ccc(-c4ccccc4)c4c3oc3ccccc34)cc2)cc1 ; [2H]c1c([2H])c([2H])c2c(sc3c([2H])c([2H])c(-c4ccc(N(c5ccc(-c6ccccc6)cc5)c5ccc(-c6ccccc6)c6c5oc5ccccc56)cc4)c([2H])c32)c1[2H]]

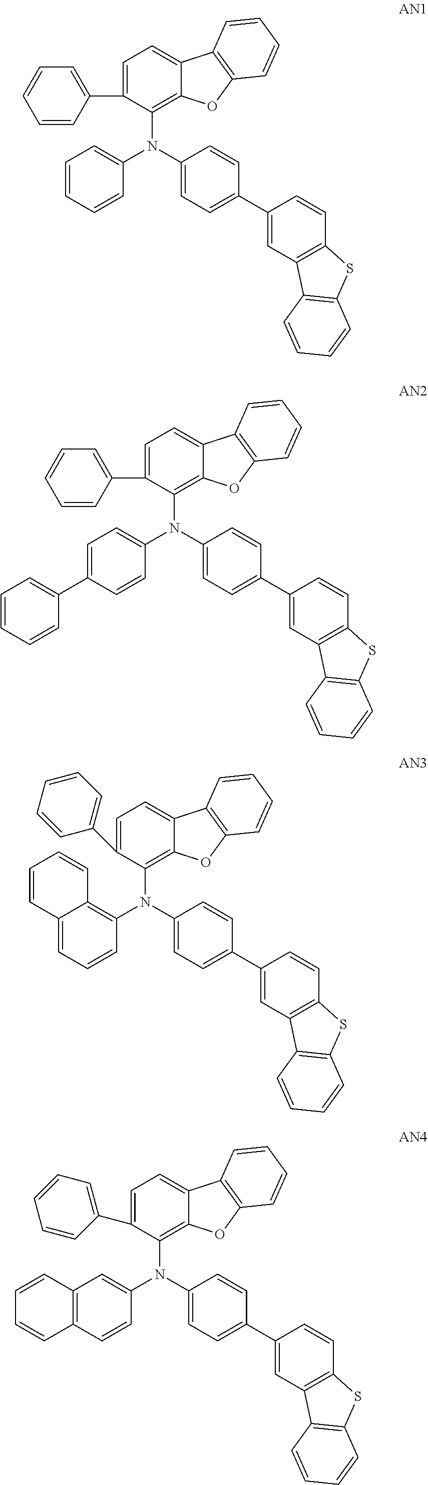

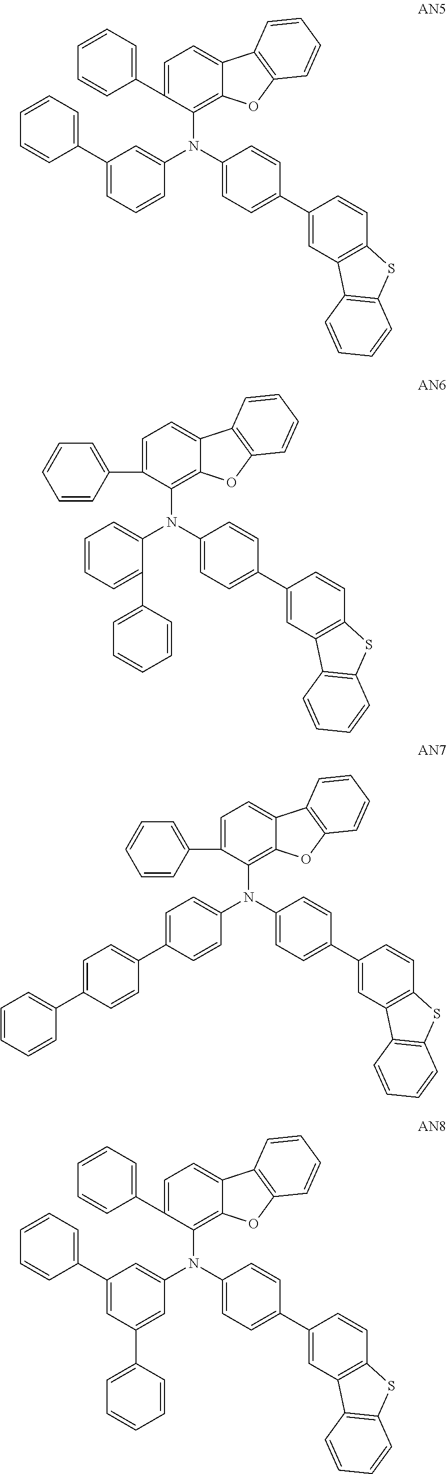

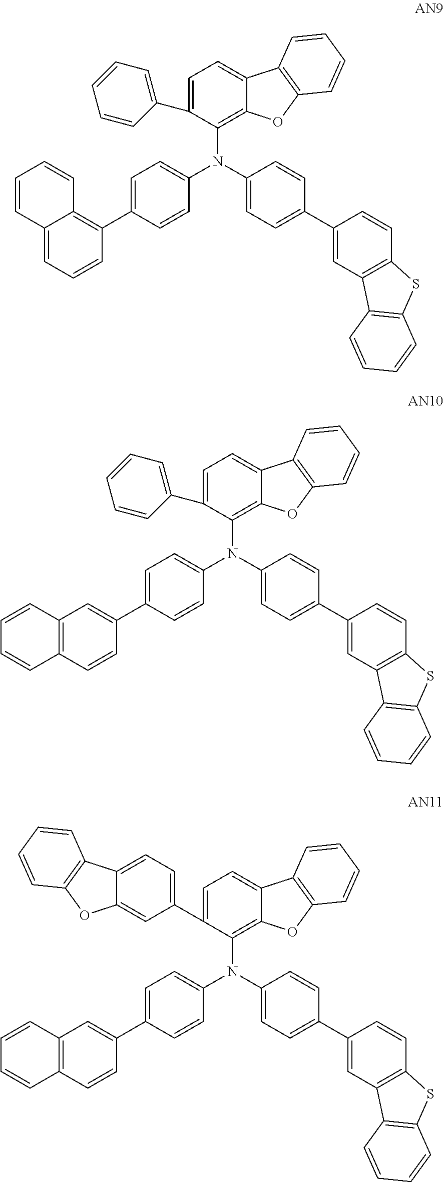 [SMILES: c1ccc(-c2ccc3c(oc4ccccc43)c2N(c2ccc(-c3ccc4sc5ccccc5c4c3)cc2)c2ccc(-c3cccc4ccccc34)cc2)cc1 ; c1ccc(-c2ccc3c(oc4ccccc43)c2N(c2ccc(-c3ccc4ccccc4c3)cc2)c2ccc(-c3ccc4sc5ccccc5c4c3)cc2)cc1 ; c1ccc2cc(-c3ccc(N(c4ccc(-c5ccc6sc7ccccc7c6c5)cc4)c4c(-c5ccc6c(c5)oc5ccccc56)ccc5c4oc4ccccc45)cc3)ccc2c1]

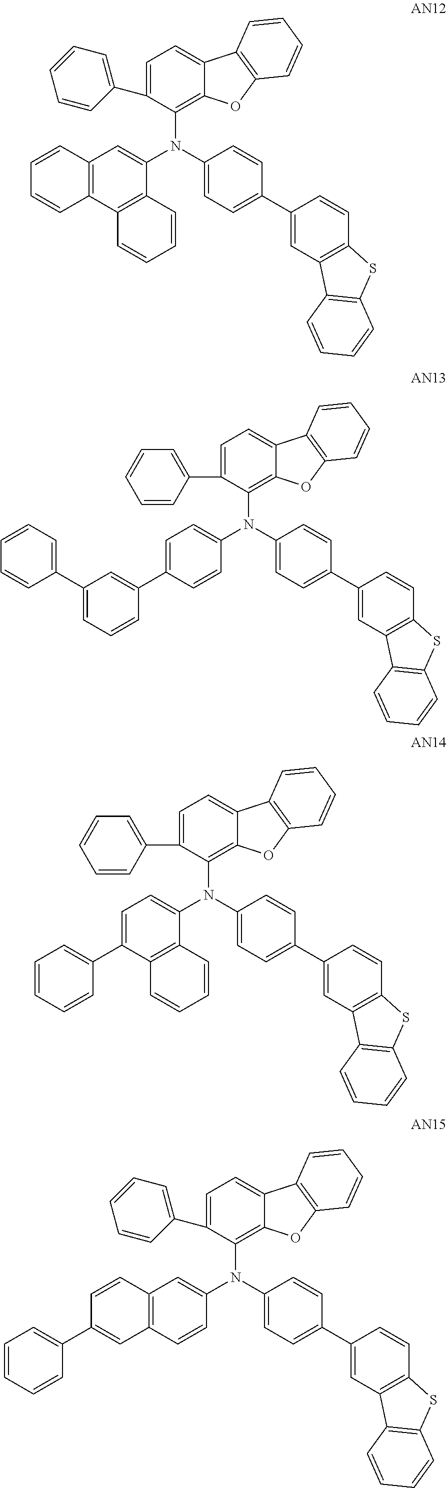 [SMILES: c1ccc(-c2ccc3c(oc4ccccc43)c2N(c2ccc(-c3ccc4sc5ccccc5c4c3)cc2)c2cc3ccccc3c3ccccc23)cc1 ; c1ccc(-c2cccc(-c3ccc(N(c4ccc(-c5ccc6sc7ccccc7c6c5)cc4)c4c(-c5ccccc5)ccc5c4oc4ccccc45)cc3)c2)cc1 ; c1ccc(-c2ccc3c(oc4ccccc43)c2N(c2ccc(-c3ccc4sc5ccccc5c4c3)cc2)c2ccc(-c3ccccc3)c3ccccc23)cc1 ; c1ccc(-c2ccc3cc(N(c4ccc(-c5ccc6sc7ccccc7c6c5)cc4)c4c(-c5ccccc5)ccc5c4oc4ccccc45)ccc3c2)cc1]

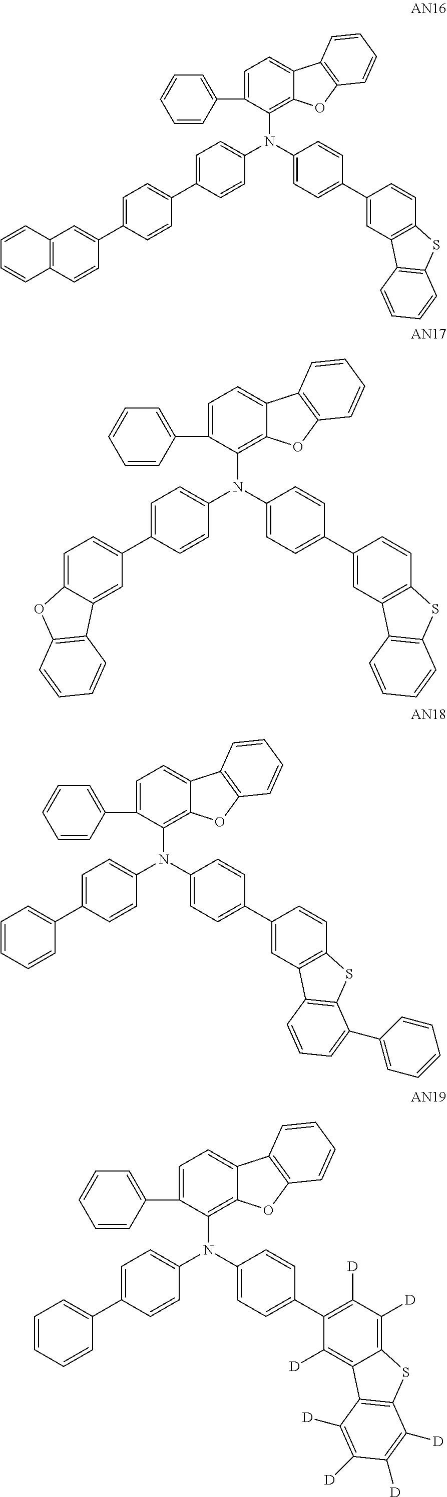 [SMILES: c1ccc(-c2ccc3c(oc4ccccc43)c2N(c2ccc(-c3ccc(-c4ccc5ccccc5c4)cc3)cc2)c2ccc(-c3ccc4sc5ccccc5c4c3)cc2)cc1 ; c1ccc(-c2ccc3c(oc4ccccc43)c2N(c2ccc(-c3ccc4oc5ccccc5c4c3)cc2)c2ccc(-c3ccc4sc5ccccc5c4c3)cc2)cc1 ; c1ccc(-c2ccc(N(c3ccc(-c4ccc5sc6c(-c7ccccc7)cccc6c5c4)cc3)c3c(-c4ccccc4)ccc4c3oc3ccccc34)cc2)cc1 ; [2H]c1c([2H])c([2H])c2c(sc3c([2H])c([2H])c(-c4ccc(N(c5ccc(-c6ccccc6)cc5)c5c(-c6ccccc6)ccc6c5oc5ccccc56)cc4)c([2H])c32)c1[2H]]

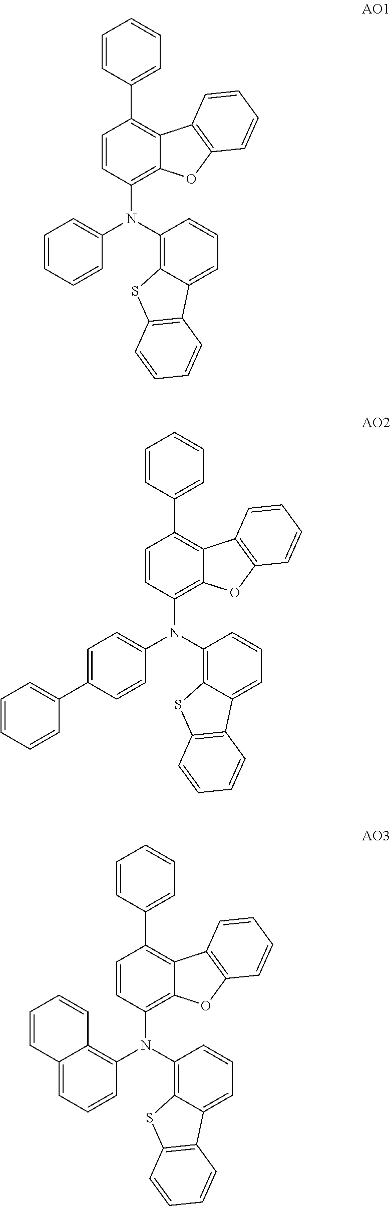 [SMILES: c1ccc(-c2ccc(N(c3ccccc3)c3cccc4c3sc3ccccc34)c3oc4ccccc4c23)cc1 ; c1ccc(-c2ccc(N(c3ccc(-c4ccccc4)c4c3oc3ccccc34)c3cccc4c3sc3ccccc34)cc2)cc1 ; c1ccc(-c2ccc(N(c3cccc4ccccc34)c3cccc4c3sc3ccccc34)c3oc4ccccc4c23)cc1]

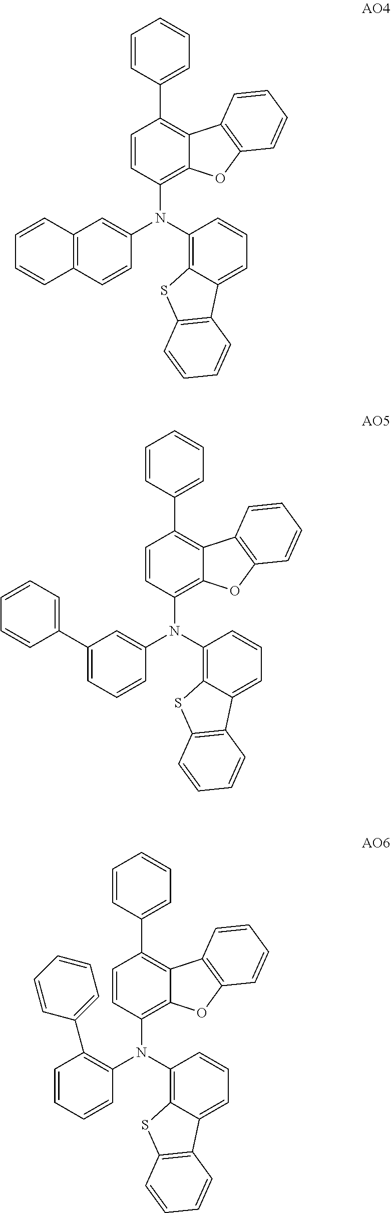 [SMILES: c1ccc(-c2ccc(N(c3ccc4ccccc4c3)c3cccc4c3sc3ccccc34)c3oc4ccccc4c23)cc1 ; c1ccc(-c2cccc(N(c3ccc(-c4ccccc4)c4c3oc3ccccc34)c3cccc4c3sc3ccccc34)c2)cc1 ; c1ccc(-c2ccccc2N(c2ccc(-c3ccccc3)c3c2oc2ccccc23)c2cccc3c2sc2ccccc23)cc1]

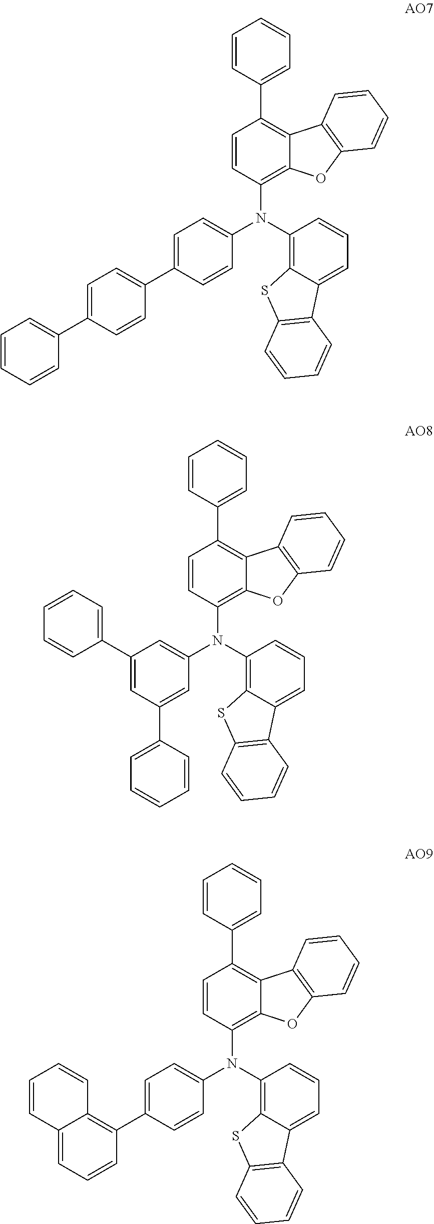 [SMILES: c1ccc(-c2ccc(-c3ccc(N(c4ccc(-c5ccccc5)c5c4oc4ccccc45)c4cccc5c4sc4ccccc45)cc3)cc2)cc1 ; c1ccc(-c2cc(-c3ccccc3)cc(N(c3ccc(-c4ccccc4)c4c3oc3ccccc34)c3cccc4c3sc3ccccc34)c2)cc1 ; c1ccc(-c2ccc(N(c3ccc(-c4cccc5ccccc45)cc3)c3cccc4c3sc3ccccc34)c3oc4ccccc4c23)cc1]

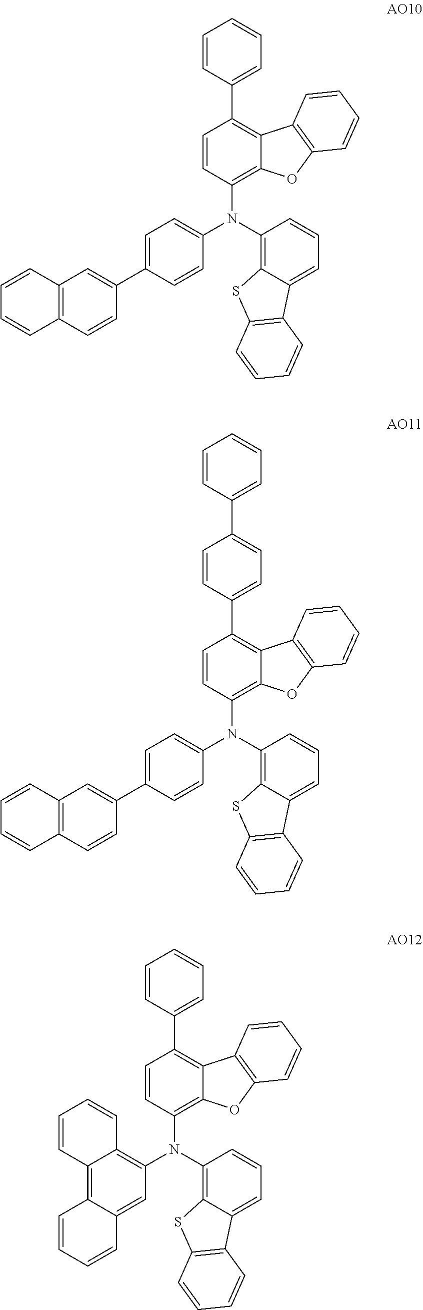 [SMILES: c1ccc(-c2ccc(N(c3ccc(-c4ccc5ccccc5c4)cc3)c3cccc4c3sc3ccccc34)c3oc4ccccc4c23)cc1 ; c1ccc(-c2ccc(-c3ccc(N(c4ccc(-c5ccc6ccccc6c5)cc4)c4cccc5c4sc4ccccc45)c4oc5ccccc5c34)cc2)cc1 ; c1ccc(-c2ccc(N(c3cc4ccccc4c4ccccc34)c3cccc4c3sc3ccccc34)c3oc4ccccc4c23)cc1]

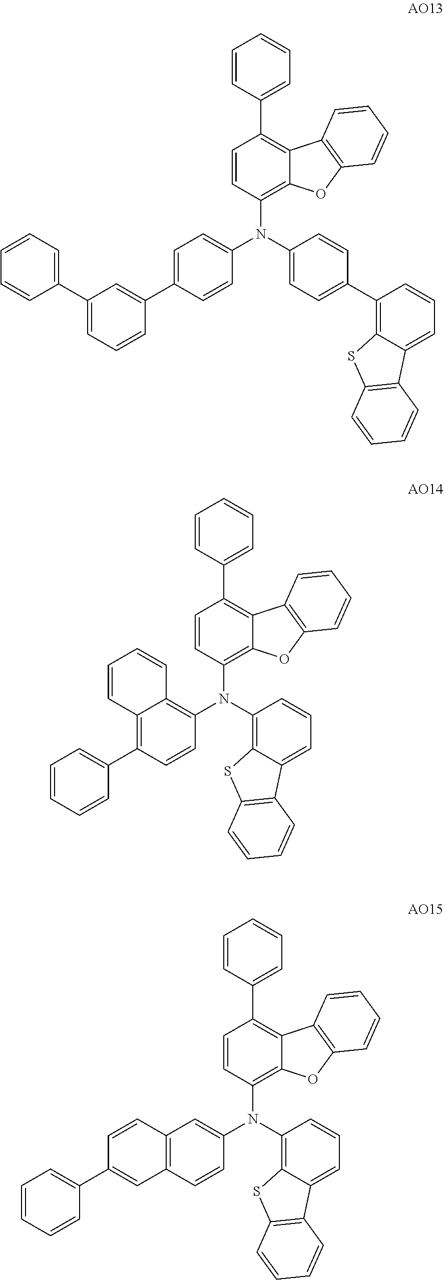 [SMILES: c1ccc(-c2cccc(-c3ccc(N(c4ccc(-c5cccc6c5sc5ccccc56)cc4)c4ccc(-c5ccccc5)c5c4oc4ccccc45)cc3)c2)cc1 ; c1ccc(-c2ccc(N(c3ccc(-c4ccccc4)c4c3oc3ccccc34)c3cccc4c3sc3ccccc34)c3ccccc23)cc1 ; c1ccc(-c2ccc3cc(N(c4ccc(-c5ccccc5)c5c4oc4ccccc45)c4cccc5c4sc4ccccc45)ccc3c2)cc1]

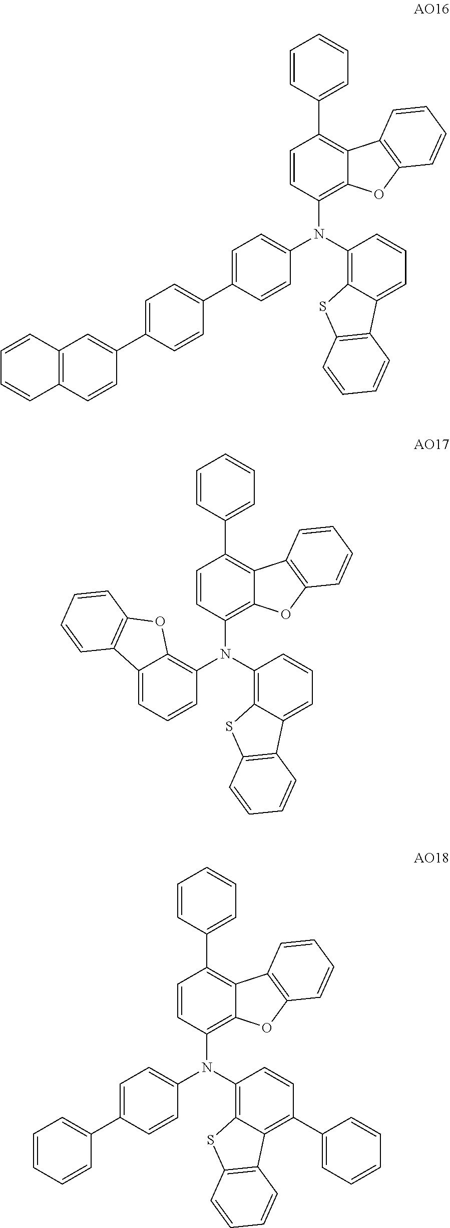 [SMILES: c1ccc(-c2ccc(N(c3ccc(-c4ccc(-c5ccc6ccccc6c5)cc4)cc3)c3cccc4c3sc3ccccc34)c3oc4ccccc4c23)cc1 ; c1ccc(-c2ccc(N(c3cccc4c3oc3ccccc34)c3cccc4c3sc3ccccc34)c3oc4ccccc4c23)cc1 ; c1ccc(-c2ccc(N(c3ccc(-c4ccccc4)c4c3oc3ccccc34)c3ccc(-c4ccccc4)c4c3sc3ccccc34)cc2)cc1]

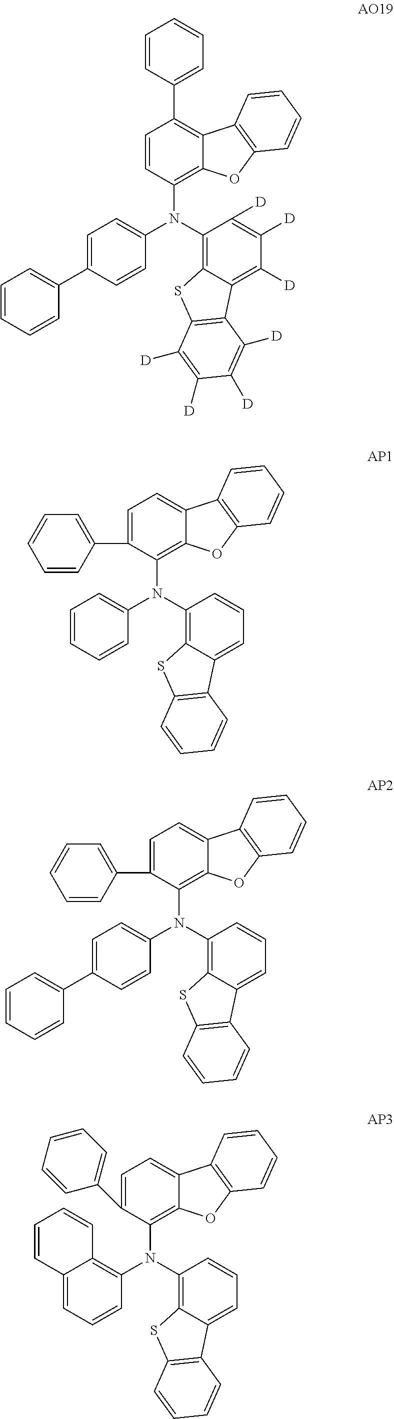 [SMILES: [2H]c1c([2H])c([2H])c2c(sc3c(N(c4ccc(-c5ccccc5)cc4)c4ccc(-c5ccccc5)c5c4oc4ccccc45)c([2H])c([2H])c([2H])c32)c1[2H] ; c1ccc(-c2ccc3c(oc4ccccc43)c2N(c2ccccc2)c2cccc3c2sc2ccccc23)cc1 ; c1ccc(-c2ccc(N(c3c(-c4ccccc4)ccc4c3oc3ccccc34)c3cccc4c3sc3ccccc34)cc2)cc1 ; c1ccc(-c2ccc3c(oc4ccccc43)c2N(c2cccc3ccccc23)c2cccc3c2sc2ccccc23)cc1]

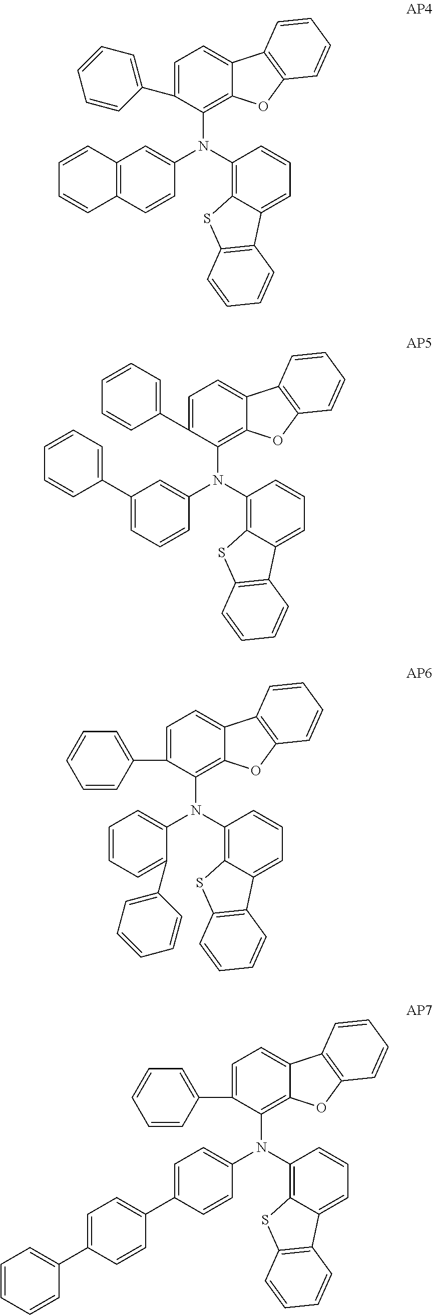 [SMILES: c1ccc(-c2ccc3c(oc4ccccc43)c2N(c2ccc3ccccc3c2)c2cccc3c2sc2ccccc23)cc1 ; c1ccc(-c2cccc(N(c3c(-c4ccccc4)ccc4c3oc3ccccc34)c3cccc4c3sc3ccccc34)c2)cc1 ; c1ccc(-c2ccccc2N(c2c(-c3ccccc3)ccc3c2oc2ccccc23)c2cccc3c2sc2ccccc23)cc1 ; c1ccc(-c2ccc(-c3ccc(N(c4c(-c5ccccc5)ccc5c4oc4ccccc45)c4cccc5c4sc4ccccc45)cc3)cc2)cc1]

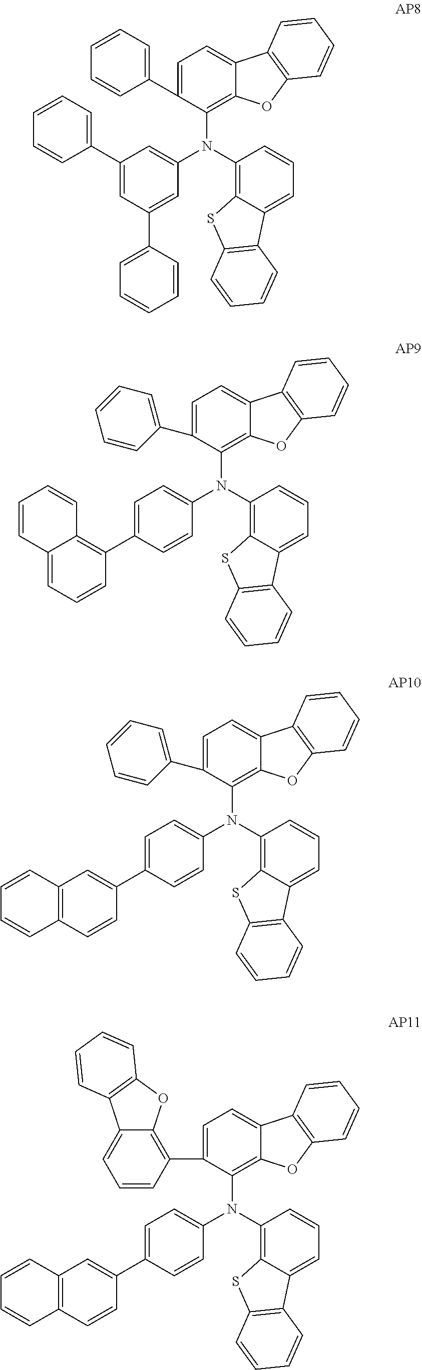 [SMILES: c1ccc(-c2cc(-c3ccccc3)cc(N(c3c(-c4ccccc4)ccc4c3oc3ccccc34)c3cccc4c3sc3ccccc34)c2)cc1 ; c1ccc(-c2ccc3c(oc4ccccc43)c2N(c2ccc(-c3cccc4ccccc34)cc2)c2cccc3c2sc2ccccc23)cc1 ; c1ccc(-c2ccc3c(oc4ccccc43)c2N(c2ccc(-c3ccc4ccccc4c3)cc2)c2cccc3c2sc2ccccc23)cc1 ; c1ccc2cc(-c3ccc(N(c4c(-c5cccc6c5oc5ccccc56)ccc5c4oc4ccccc45)c4cccc5c4sc4ccccc45)cc3)ccc2c1]

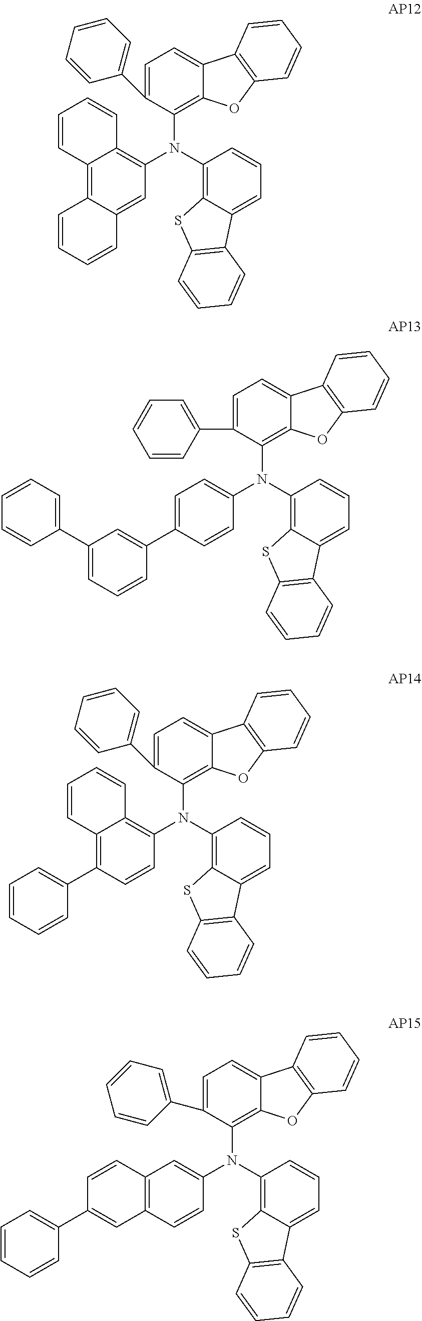 [SMILES: c1ccc(-c2ccc3c(oc4ccccc43)c2N(c2cc3ccccc3c3ccccc23)c2cccc3c2sc2ccccc23)cc1 ; c1ccc(-c2cccc(-c3ccc(N(c4c(-c5ccccc5)ccc5c4oc4ccccc45)c4cccc5c4sc4ccccc45)cc3)c2)cc1 ; c1ccc(-c2ccc3c(oc4ccccc43)c2N(c2ccc(-c3ccccc3)c3ccccc23)c2cccc3c2sc2ccccc23)cc1 ; c1ccc(-c2ccc3cc(N(c4c(-c5ccccc5)ccc5c4oc4ccccc45)c4cccc5c4sc4ccccc45)ccc3c2)cc1]

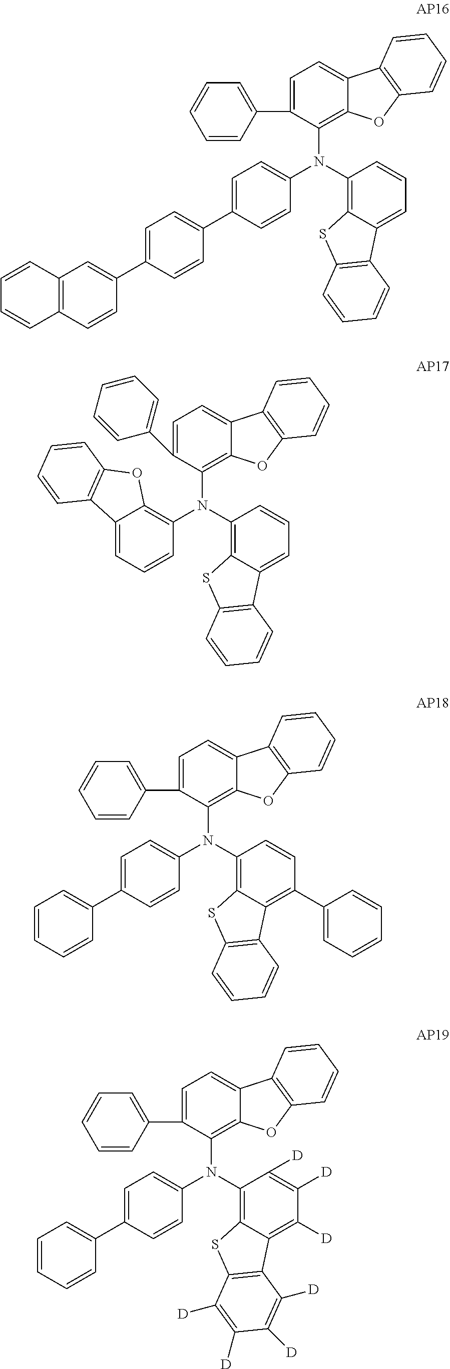 [SMILES: c1ccc(-c2ccc3c(oc4ccccc43)c2N(c2ccc(-c3ccc(-c4ccc5ccccc5c4)cc3)cc2)c2cccc3c2sc2ccccc23)cc1 ; c1ccc(-c2ccc3c(oc4ccccc43)c2N(c2cccc3c2oc2ccccc23)c2cccc3c2sc2ccccc23)cc1 ; c1ccc(-c2ccc(N(c3c(-c4ccccc4)ccc4c3oc3ccccc34)c3ccc(-c4ccccc4)c4c3sc3ccccc34)cc2)cc1 ; [2H]c1c([2H])c([2H])c2c(sc3c(N(c4ccc(-c5ccccc5)cc4)c4c(-c5ccccc5)ccc5c4oc4ccccc45)c([2H])c([2H])c([2H])c32)c1[2H]]

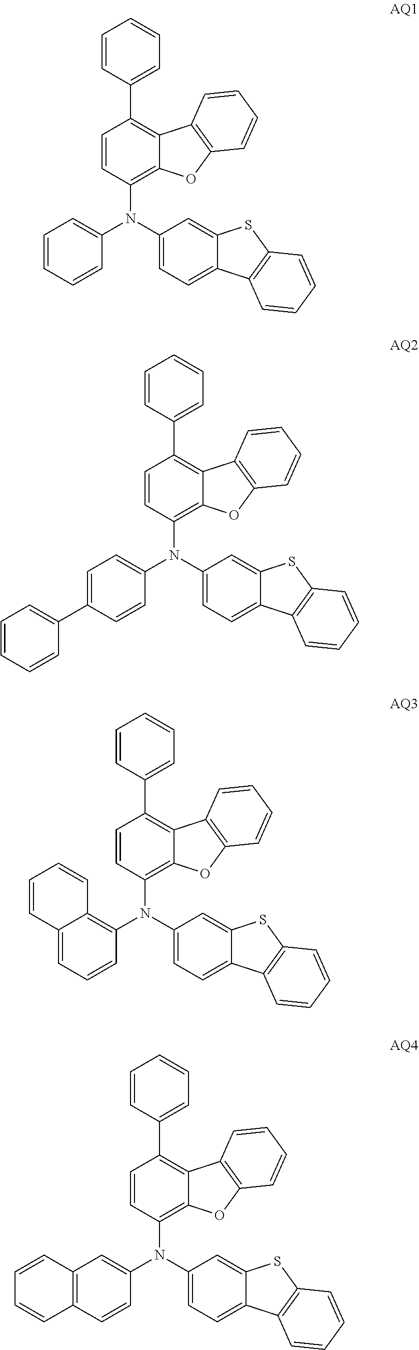 [SMILES: c1ccc(-c2ccc(N(c3ccccc3)c3ccc4c(c3)sc3ccccc34)c3oc4ccccc4c23)cc1 ; c1ccc(-c2ccc(N(c3ccc4c(c3)sc3ccccc34)c3ccc(-c4ccccc4)c4c3oc3ccccc34)cc2)cc1 ; c1ccc(-c2ccc(N(c3ccc4c(c3)sc3ccccc34)c3cccc4ccccc34)c3oc4ccccc4c23)cc1 ; c1ccc(-c2ccc(N(c3ccc4ccccc4c3)c3ccc4c(c3)sc3ccccc34)c3oc4ccccc4c23)cc1]

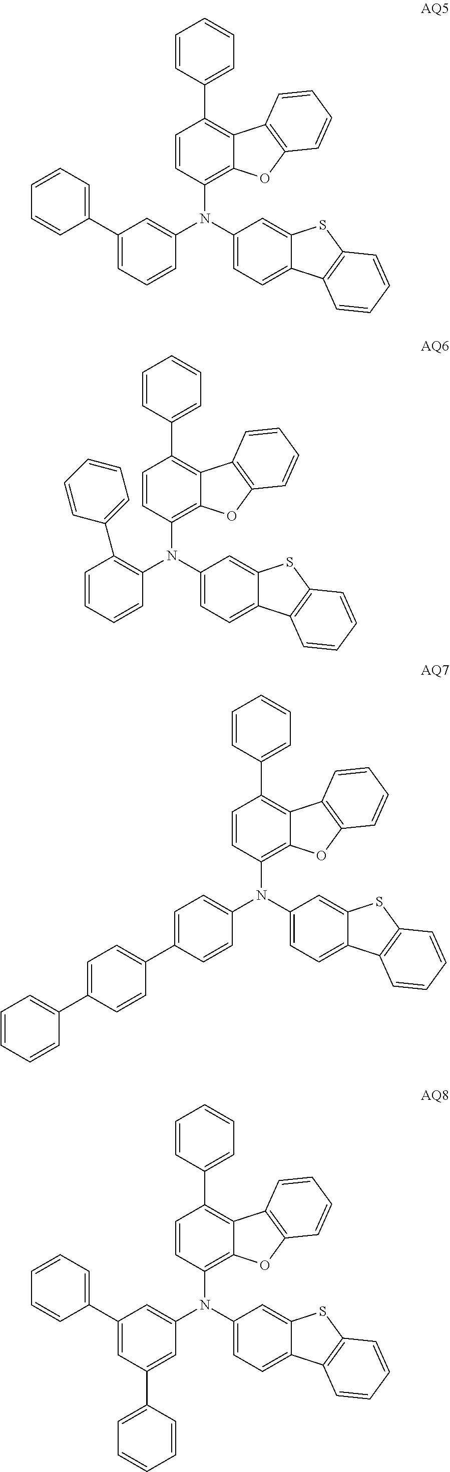 [SMILES: c1ccc(-c2cccc(N(c3ccc4c(c3)sc3ccccc34)c3ccc(-c4ccccc4)c4c3oc3ccccc34)c2)cc1 ; c1ccc(-c2ccccc2N(c2ccc3c(c2)sc2ccccc23)c2ccc(-c3ccccc3)c3c2oc2ccccc23)cc1 ; c1ccc(-c2ccc(-c3ccc(N(c4ccc5c(c4)sc4ccccc45)c4ccc(-c5ccccc5)c5c4oc4ccccc45)cc3)cc2)cc1 ; c1ccc(-c2cc(-c3ccccc3)cc(N(c3ccc4c(c3)sc3ccccc34)c3ccc(-c4ccccc4)c4c3oc3ccccc34)c2)cc1]

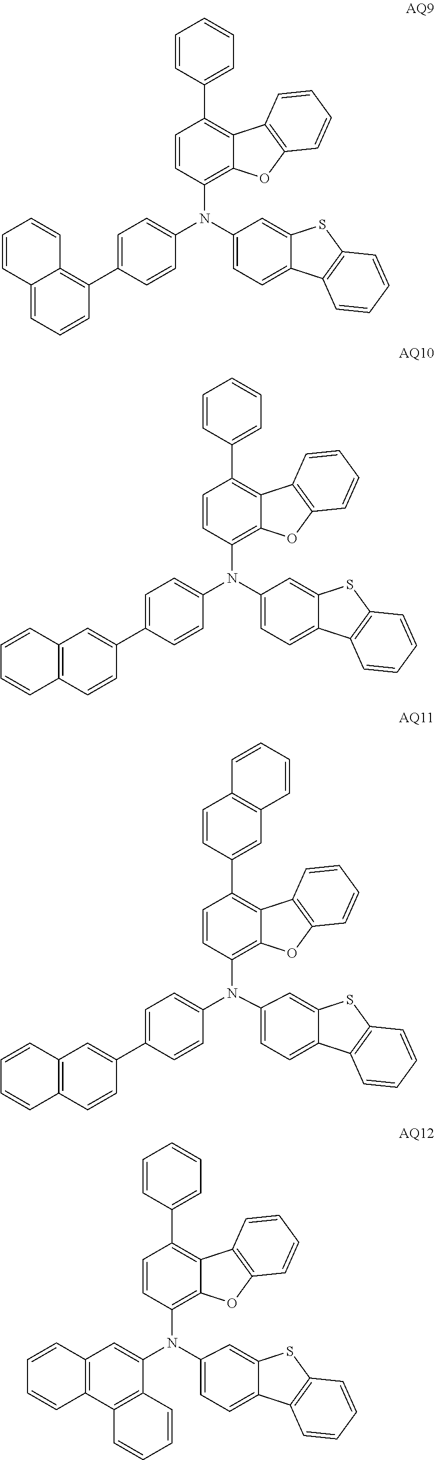 [SMILES: c1ccc(-c2ccc(N(c3ccc(-c4cccc5ccccc45)cc3)c3ccc4c(c3)sc3ccccc34)c3oc4ccccc4c23)cc1 ; c1ccc(-c2ccc(N(c3ccc(-c4ccc5ccccc5c4)cc3)c3ccc4c(c3)sc3ccccc34)c3oc4ccccc4c23)cc1 ; c1ccc2cc(-c3ccc(N(c4ccc5c(c4)sc4ccccc45)c4ccc(-c5ccc6ccccc6c5)c5c4oc4ccccc45)cc3)ccc2c1 ; c1ccc(-c2ccc(N(c3ccc4c(c3)sc3ccccc34)c3cc4ccccc4c4ccccc34)c3oc4ccccc4c23)cc1]

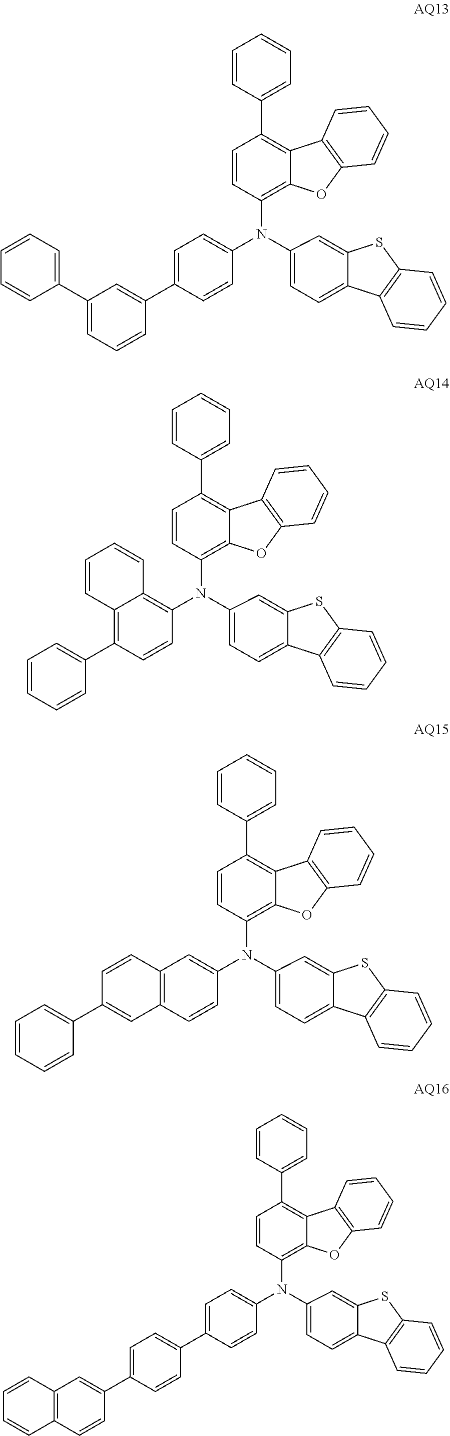 [SMILES: c1ccc(-c2cccc(-c3ccc(N(c4ccc5c(c4)sc4ccccc45)c4ccc(-c5ccccc5)c5c4oc4ccccc45)cc3)c2)cc1 ; c1ccc(-c2ccc(N(c3ccc4c(c3)sc3ccccc34)c3ccc(-c4ccccc4)c4c3oc3ccccc34)c3ccccc23)cc1 ; c1ccc(-c2ccc3cc(N(c4ccc5c(c4)sc4ccccc45)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc4c(c3)sc3ccccc34)c3oc4ccccc4c23)cc1]

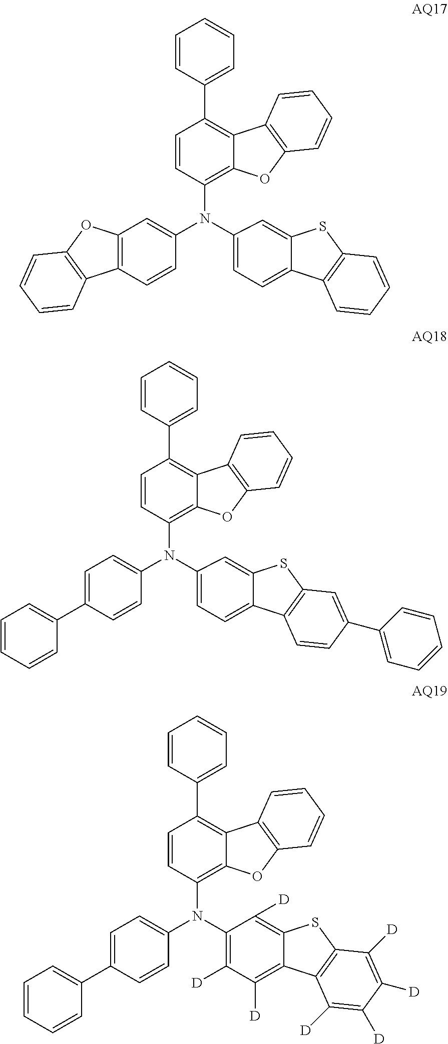 [SMILES: c1ccc(-c2ccc(N(c3ccc4c(c3)oc3ccccc34)c3ccc4c(c3)sc3ccccc34)c3oc4ccccc4c23)cc1 ; c1ccc(-c2ccc(N(c3ccc4c(c3)sc3cc(-c5ccccc5)ccc34)c3ccc(-c4ccccc4)c4c3oc3ccccc34)cc2)cc1 ; [2H]c1c([2H])c([2H])c2c(sc3c([2H])c(N(c4ccc(-c5ccccc5)cc4)c4ccc(-c5ccccc5)c5c4oc4ccccc45)c([2H])c([2H])c32)c1[2H]]

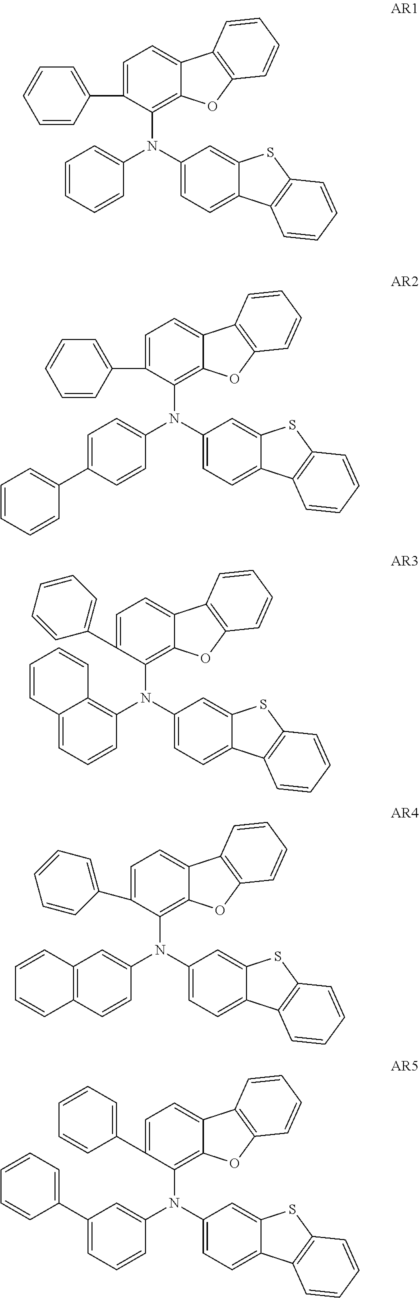

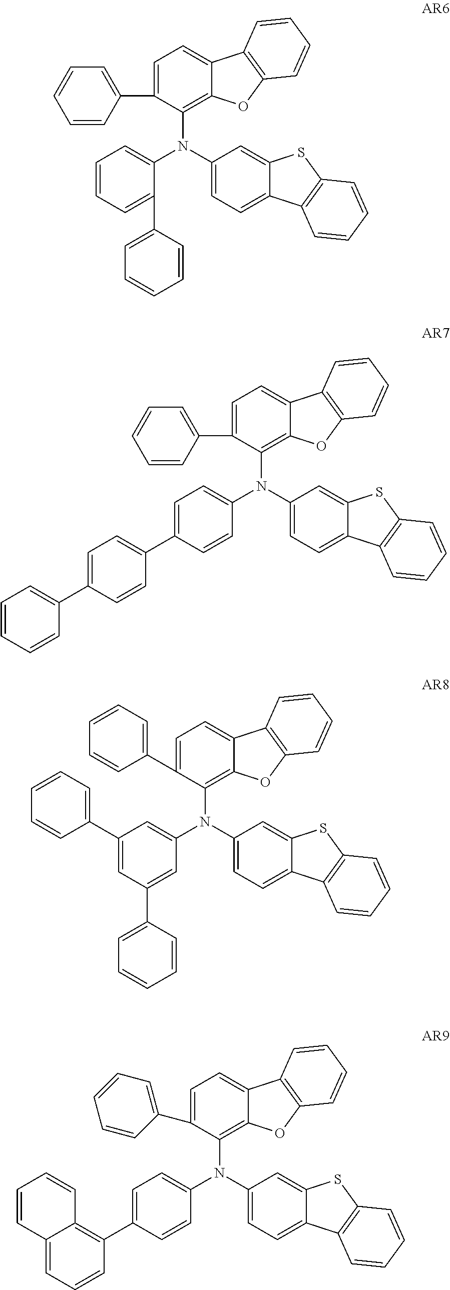 [SMILES: c1ccc(-c2ccccc2N(c2ccc3c(c2)sc2ccccc23)c2c(-c3ccccc3)ccc3c2oc2ccccc23)cc1 ; c1ccc(-c2ccc(-c3ccc(N(c4ccc5c(c4)sc4ccccc45)c4c(-c5ccccc5)ccc5c4oc4ccccc45)cc3)cc2)cc1 ; c1ccc(-c2cc(-c3ccccc3)cc(N(c3ccc4c(c3)sc3ccccc34)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc3c(c2)sc2ccccc23)cc1]

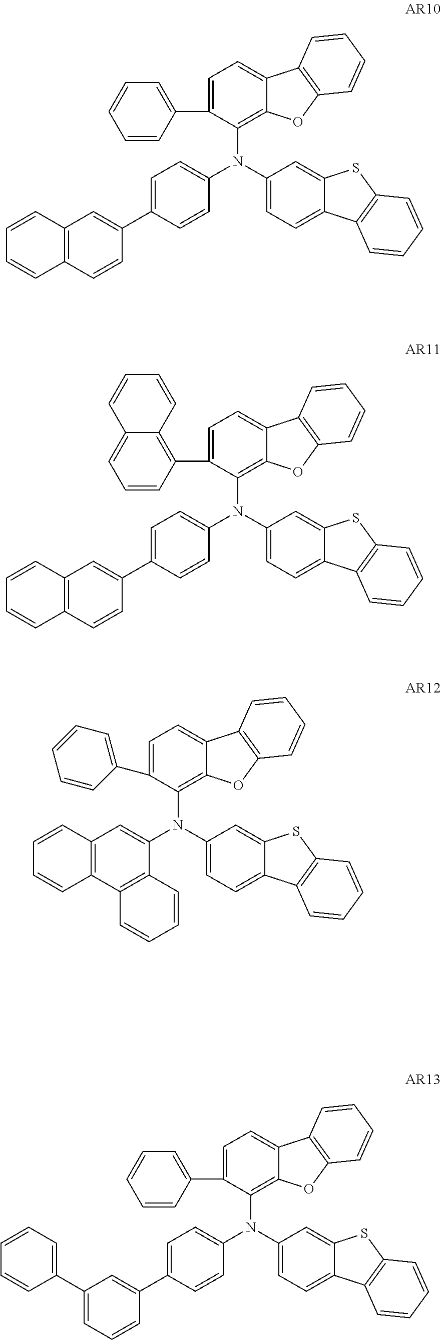 [SMILES: c1ccc(-c2ccc3c(oc4ccccc43)c2N(c2ccc(-c3ccc4ccccc4c3)cc2)c2ccc3c(c2)sc2ccccc23)cc1 ; c1ccc2cc(-c3ccc(N(c4ccc5c(c4)sc4ccccc45)c4c(-c5cccc6ccccc56)ccc5c4oc4ccccc45)cc3)ccc2c1 ; c1ccc(-c2ccc3c(oc4ccccc43)c2N(c2ccc3c(c2)sc2ccccc23)c2cc3ccccc3c3ccccc23)cc1 ; c1ccc(-c2cccc(-c3ccc(N(c4ccc5c(c4)sc4ccccc45)c4c(-c5ccccc5)ccc5c4oc4ccccc45)cc3)c2)cc1]

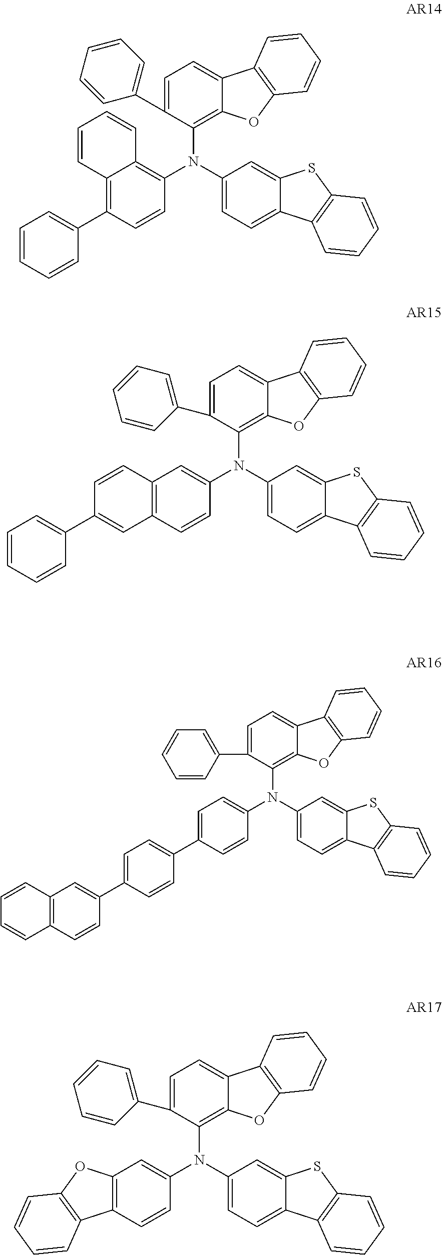 [SMILES: c1ccc(-c2ccc3c(oc4ccccc43)c2N(c2ccc3c(c2)sc2ccccc23)c2ccc(-c3ccccc3)c3ccccc23)cc1 ; c1ccc(-c2ccc3cc(N(c4ccc5c(c4)sc4ccccc45)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc3c(c2)sc2ccccc23)cc1 ; c1ccc(-c2ccc3c(oc4ccccc43)c2N(c2ccc3c(c2)oc2ccccc23)c2ccc3c(c2)sc2ccccc23)cc1]

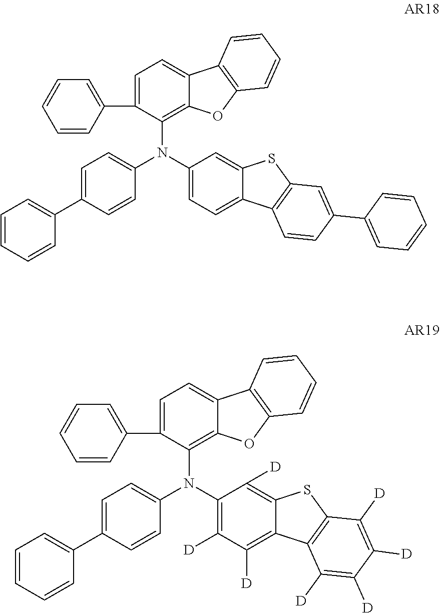 [SMILES: c1ccc(-c2ccc(N(c3ccc4c(c3)sc3cc(-c5ccccc5)ccc34)c3c(-c4ccccc4)ccc4c3oc3ccccc34)cc2)cc1 ; [2H]c1c([2H])c([2H])c2c(sc3c([2H])c(N(c4ccc(-c5ccccc5)cc4)c4c(-c5ccccc5)ccc5c4oc4ccccc45)c([2H])c([2H])c32)c1[2H]]

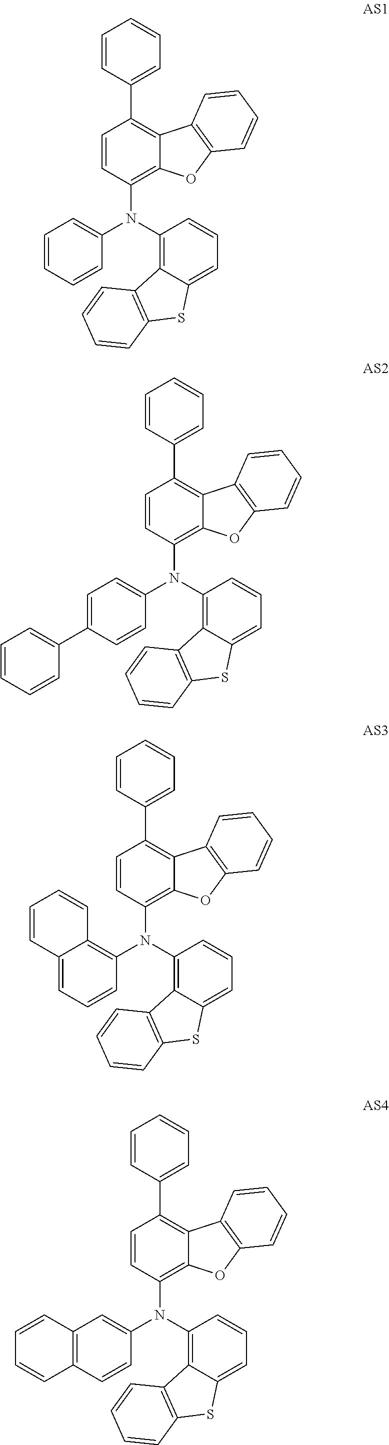

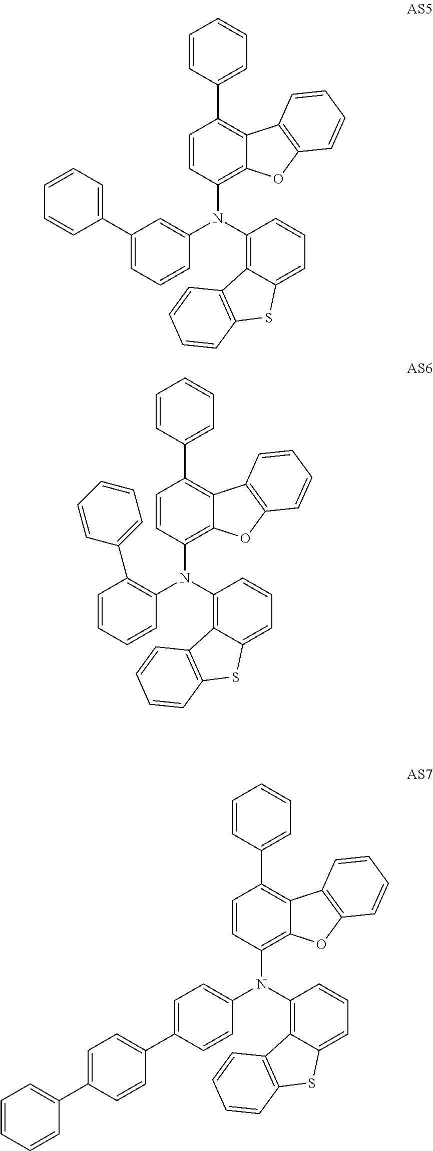

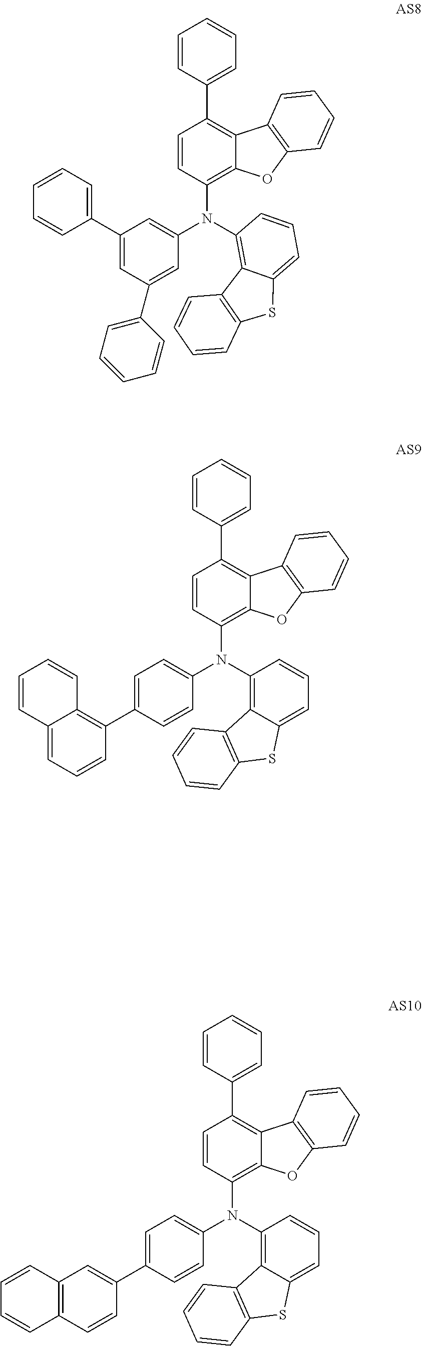 [SMILES: c1ccc(-c2cc(-c3ccccc3)cc(N(c3ccc(-c4ccccc4)c4c3oc3ccccc34)c3cccc4sc5ccccc5c34)c2)cc1 ; c1ccc(-c2ccc(N(c3ccc(-c4cccc5ccccc45)cc3)c3cccc4sc5ccccc5c34)c3oc4ccccc4c23)cc1 ; c1ccc(-c2ccc(N(c3ccc(-c4ccc5ccccc5c4)cc3)c3cccc4sc5ccccc5c34)c3oc4ccccc4c23)cc1]

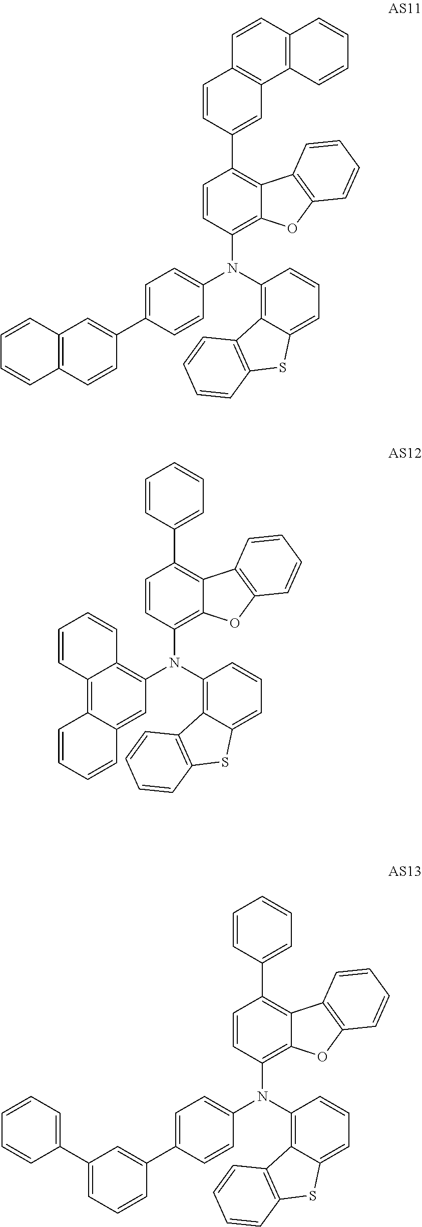 [SMILES: c1ccc2cc(-c3ccc(N(c4ccc(-c5ccc6ccc7ccccc7c6c5)c5c4oc4ccccc45)c4cccc5sc6ccccc6c45)cc3)ccc2c1 ; c1ccc(-c2ccc(N(c3cc4ccccc4c4ccccc34)c3cccc4sc5ccccc5c34)c3oc4ccccc4c23)cc1 ; c1ccc(-c2cccc(-c3ccc(N(c4ccc(-c5ccccc5)c5c4oc4ccccc45)c4cccc5sc6ccccc6c45)cc3)c2)cc1]

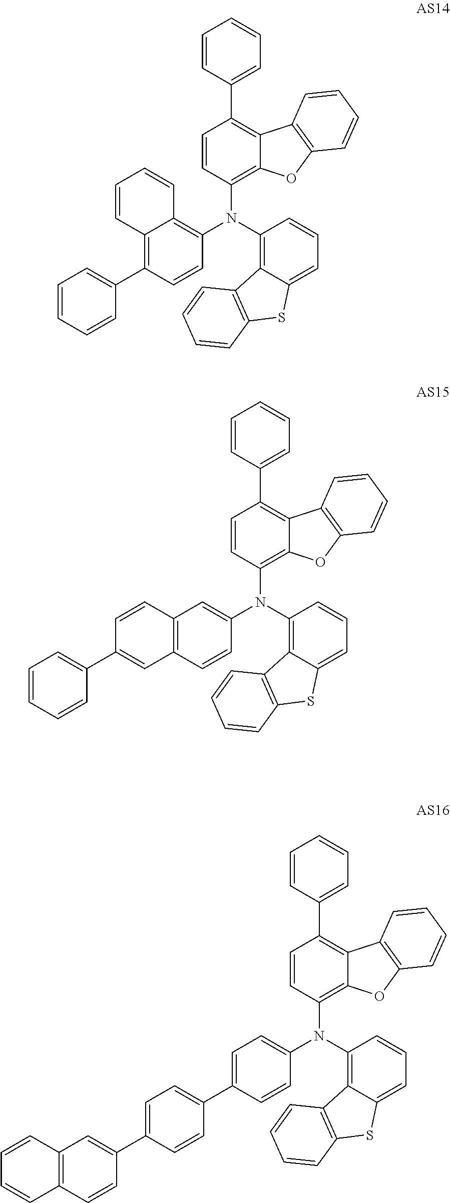 [SMILES: c1ccc(-c2ccc(N(c3ccc(-c4ccccc4)c4c3oc3ccccc34)c3cccc4sc5ccccc5c34)c3ccccc23)cc1 ; c1ccc(-c2ccc3cc(N(c4ccc(-c5ccccc5)c5c4oc4ccccc45)c4cccc5sc6ccccc6c45)ccc3c2)cc1 ; c1ccc(-c2ccc(N(c3ccc(-c4ccc(-c5ccc6ccccc6c5)cc4)cc3)c3cccc4sc5ccccc5c34)c3oc4ccccc4c23)cc1]

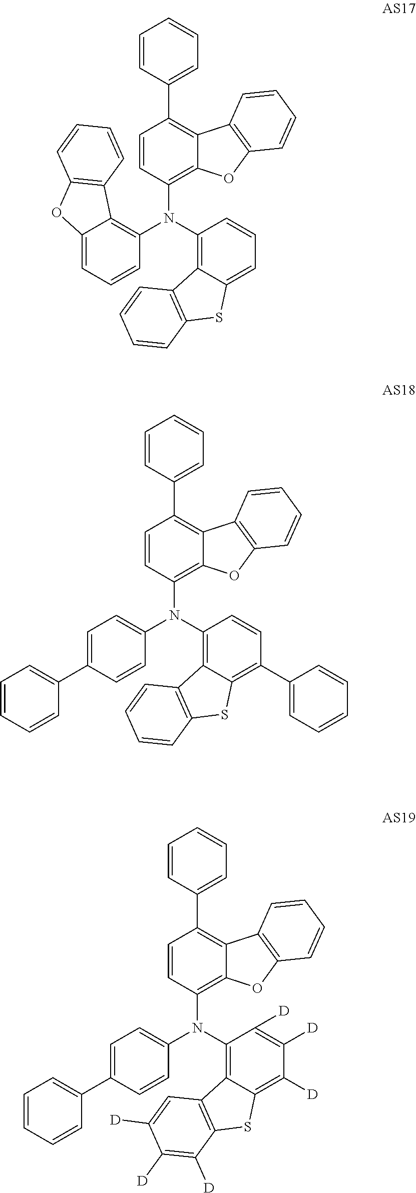 [SMILES: c1ccc(-c2ccc(N(c3cccc4oc5ccccc5c34)c3cccc4sc5ccccc5c34)c3oc4ccccc4c23)cc1 ; c1ccc(-c2ccc(N(c3ccc(-c4ccccc4)c4c3oc3ccccc34)c3ccc(-c4ccccc4)c4sc5ccccc5c34)cc2)cc1 ; [2H]c1cc2c(sc3c([2H])c([2H])c([2H])c(N(c4ccc(-c5ccccc5)cc4)c4ccc(-c5ccccc5)c5c4oc4ccccc45)c32)c([2H])c1[2H]]

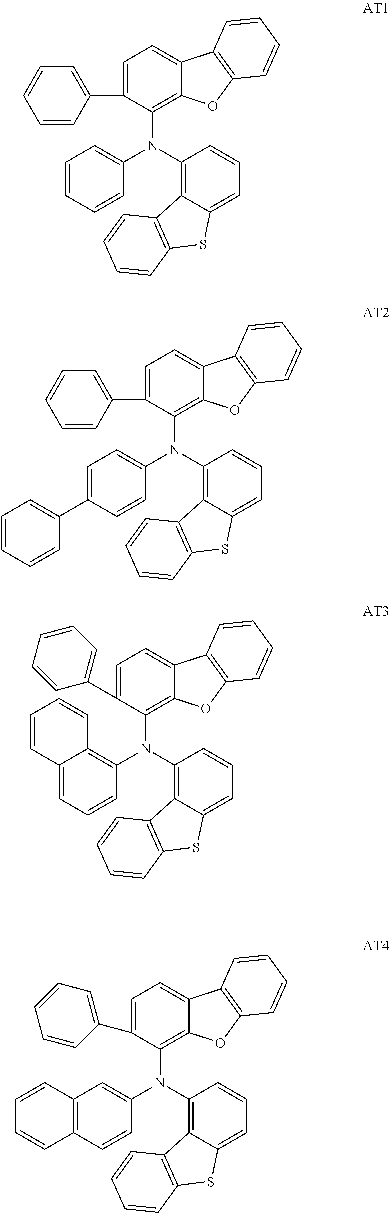 [SMILES: c1ccc(-c2ccc3c(oc4ccccc43)c2N(c2ccccc2)c2cccc3sc4ccccc4c23)cc1 ; c1ccc(-c2ccc(N(c3c(-c4ccccc4)ccc4c3oc3ccccc34)c3cccc4sc5ccccc5c34)cc2)cc1 ; c1ccc(-c2ccc3c(oc4ccccc43)c2N(c2cccc3ccccc23)c2cccc3sc4ccccc4c23)cc1 ; c1ccc(-c2ccc3c(oc4ccccc43)c2N(c2ccc3ccccc3c2)c2cccc3sc4ccccc4c23)cc1]

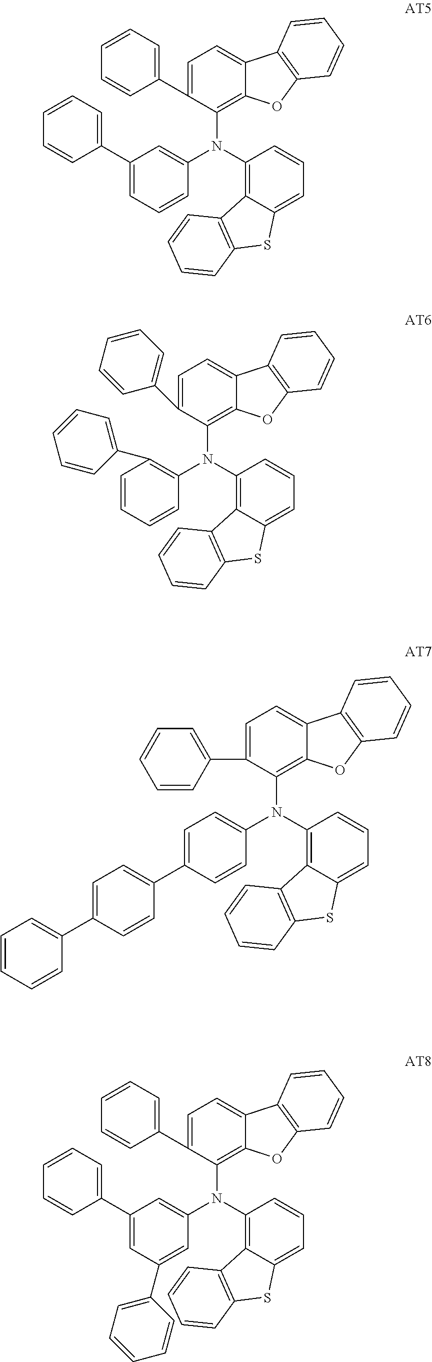 [SMILES: c1ccc(-c2cccc(N(c3c(-c4ccccc4)ccc4c3oc3ccccc34)c3cccc4sc5ccccc5c34)c2)cc1 ; c1ccc(-c2ccccc2N(c2c(-c3ccccc3)ccc3c2oc2ccccc23)c2cccc3sc4ccccc4c23)cc1 ; c1ccc(-c2ccc(-c3ccc(N(c4c(-c5ccccc5)ccc5c4oc4ccccc45)c4cccc5sc6ccccc6c45)cc3)cc2)cc1 ; c1ccc(-c2cc(-c3ccccc3)cc(N(c3c(-c4ccccc4)ccc4c3oc3ccccc34)c3cccc4sc5ccccc5c34)c2)cc1]

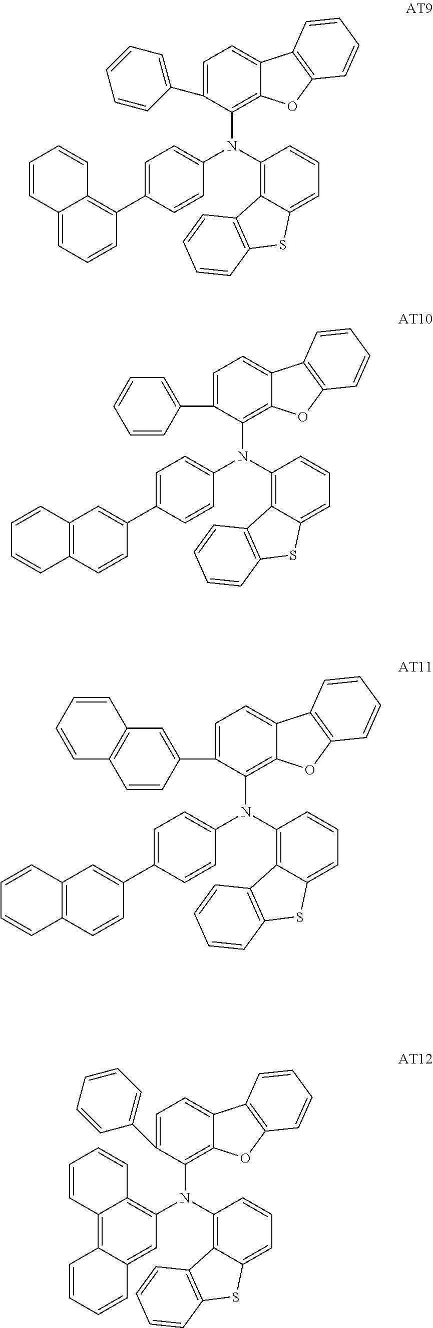 [SMILES: c1ccc(-c2ccc3c(oc4ccccc43)c2N(c2ccc(-c3cccc4ccccc34)cc2)c2cccc3sc4ccccc4c23)cc1 ; c1ccc(-c2ccc3c(oc4ccccc43)c2N(c2ccc(-c3ccc4ccccc4c3)cc2)c2cccc3sc4ccccc4c23)cc1 ; c1ccc2cc(-c3ccc(N(c4c(-c5ccc6ccccc6c5)ccc5c4oc4ccccc45)c4cccc5sc6ccccc6c45)cc3)ccc2c1 ; c1ccc(-c2ccc3c(oc4ccccc43)c2N(c2cc3ccccc3c3ccccc23)c2cccc3sc4ccccc4c23)cc1]

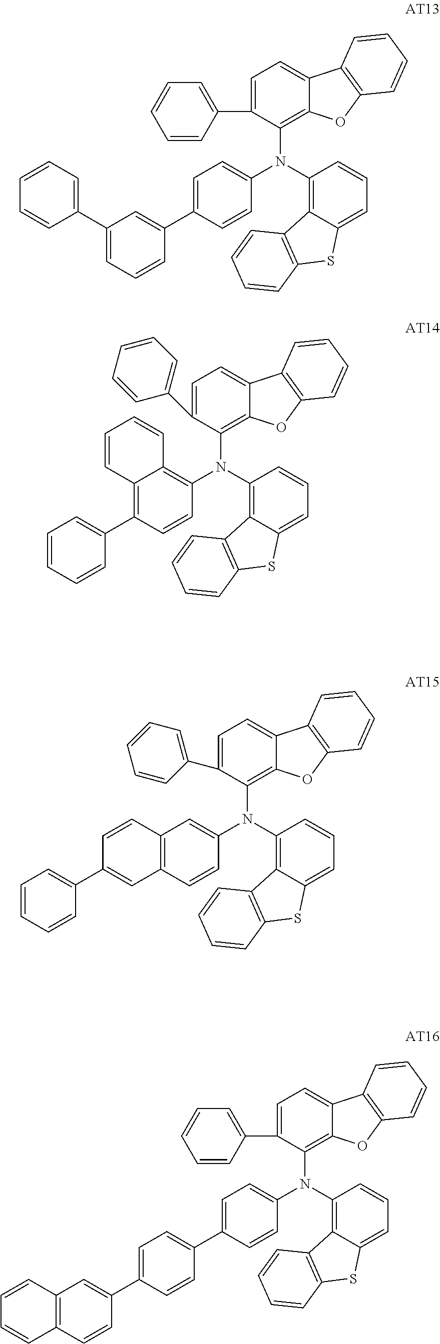 [SMILES: c1ccc(-c2cccc(-c3ccc(N(c4c(-c5ccccc5)ccc5c4oc4ccccc45)c4cccc5sc6ccccc6c45)cc3)c2)cc1 ; c1ccc(-c2ccc3c(oc4ccccc43)c2N(c2ccc(-c3ccccc3)c3ccccc23)c2cccc3sc4ccccc4c23)cc1 ; c1ccc(-c2ccc3cc(N(c4c(-c5ccccc5)ccc5c4oc4ccccc45)c4cccc5sc6ccccc6c45)ccc3c2)cc1 ; c1ccc(-c2ccc3c(oc4ccccc43)c2N(c2ccc(-c3ccc(-c4ccc5ccccc5c4)cc3)cc2)c2cccc3sc4ccccc4c23)cc1]

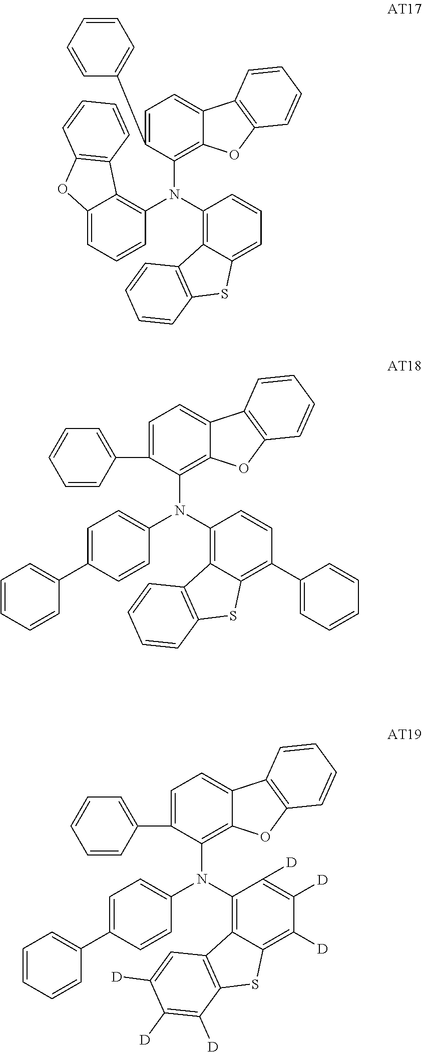 [SMILES: c1ccc(-c2ccc3c(oc4ccccc43)c2N(c2cccc3oc4ccccc4c23)c2cccc3sc4ccccc4c23)cc1 ; c1ccc(-c2ccc(N(c3c(-c4ccccc4)ccc4c3oc3ccccc34)c3ccc(-c4ccccc4)c4sc5ccccc5c34)cc2)cc1 ; [2H]c1cc2c(sc3c([2H])c([2H])c([2H])c(N(c4ccc(-c5ccccc5)cc4)c4c(-c5ccccc5)ccc5c4oc4ccccc45)c32)c([2H])c1[2H]]

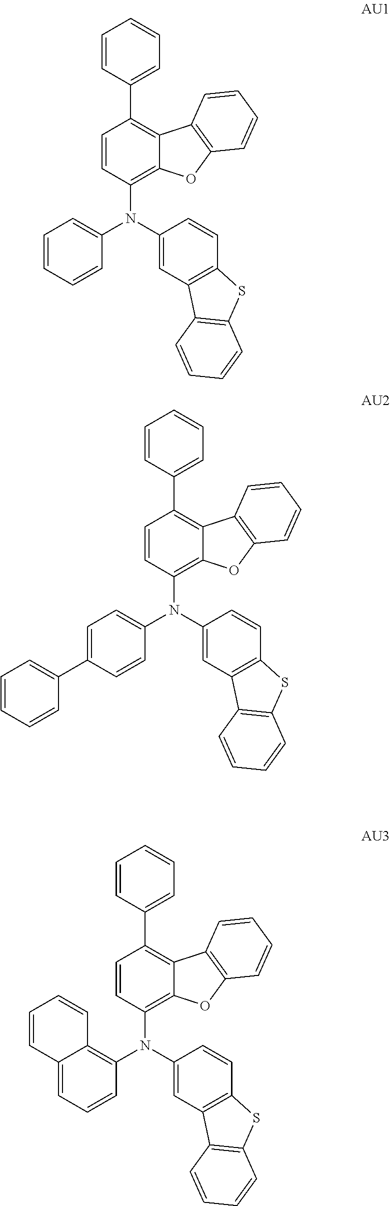

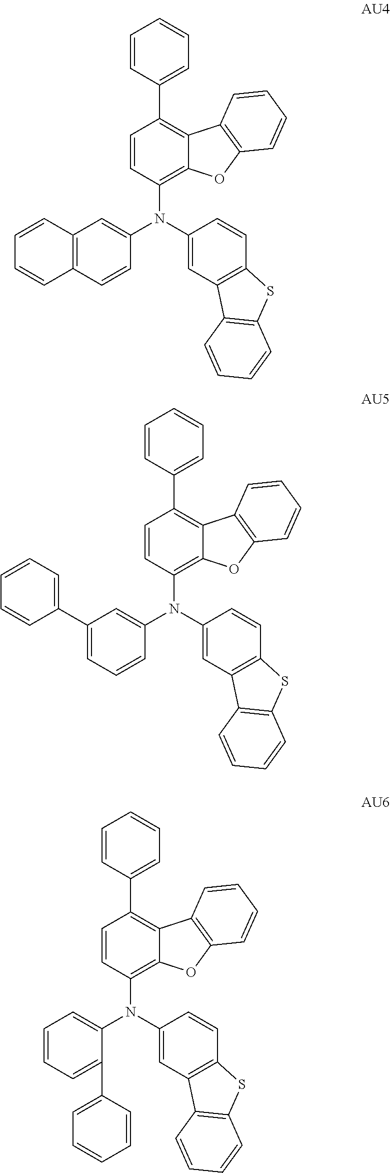 [SMILES: c1ccc(-c2ccc(N(c3ccc4ccccc4c3)c3ccc4sc5ccccc5c4c3)c3oc4ccccc4c23)cc1 ; c1ccc(-c2cccc(N(c3ccc4sc5ccccc5c4c3)c3ccc(-c4ccccc4)c4c3oc3ccccc34)c2)cc1 ; c1ccc(-c2ccccc2N(c2ccc3sc4ccccc4c3c2)c2ccc(-c3ccccc3)c3c2oc2ccccc23)cc1]

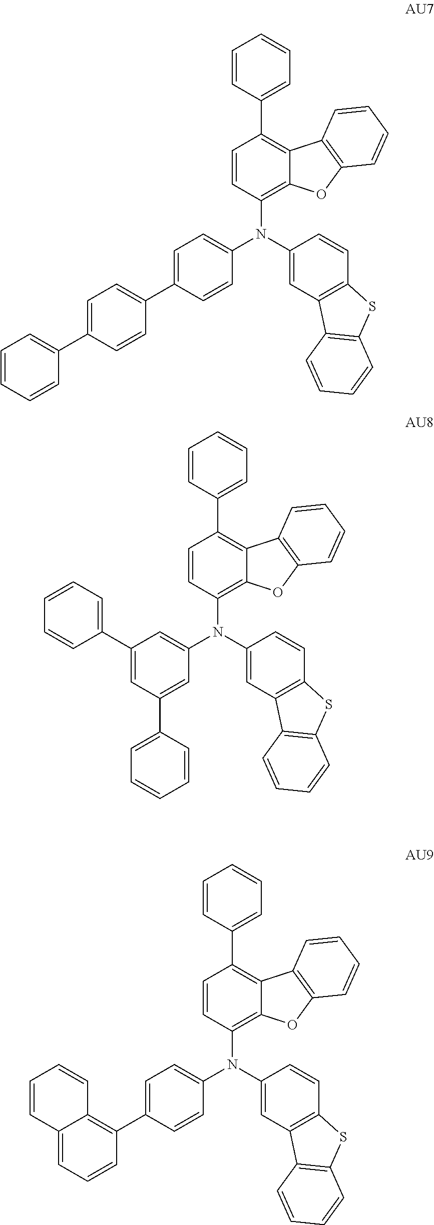 [SMILES: c1ccc(-c2ccc(-c3ccc(N(c4ccc5sc6ccccc6c5c4)c4ccc(-c5ccccc5)c5c4oc4ccccc45)cc3)cc2)cc1 ; c1ccc(-c2cc(-c3ccccc3)cc(N(c3ccc4sc5ccccc5c4c3)c3ccc(-c4ccccc4)c4c3oc3ccccc34)c2)cc1 ; c1ccc(-c2ccc(N(c3ccc(-c4cccc5ccccc45)cc3)c3ccc4sc5ccccc5c4c3)c3oc4ccccc4c23)cc1]

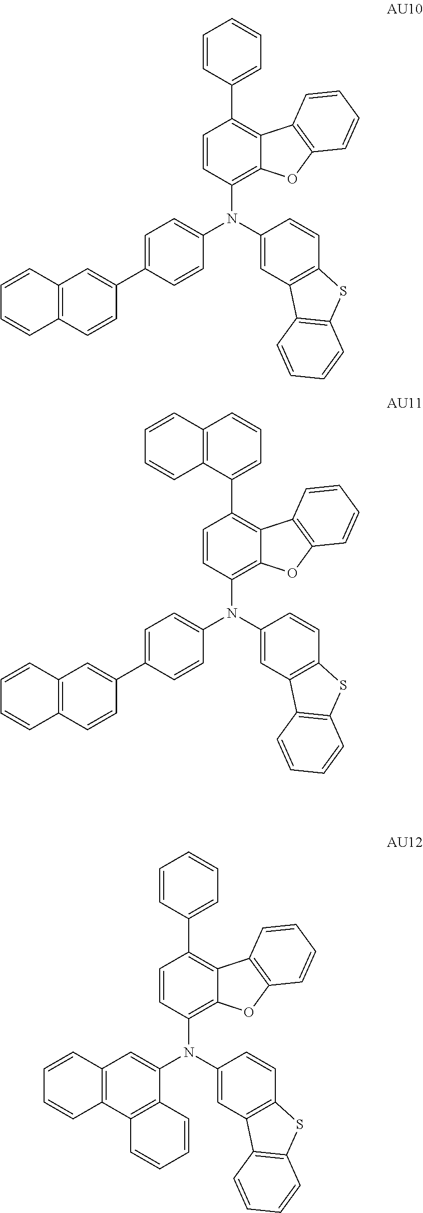 [SMILES: c1ccc(-c2ccc(N(c3ccc(-c4ccc5ccccc5c4)cc3)c3ccc4sc5ccccc5c4c3)c3oc4ccccc4c23)cc1 ; c1ccc2cc(-c3ccc(N(c4ccc5sc6ccccc6c5c4)c4ccc(-c5cccc6ccccc56)c5c4oc4ccccc45)cc3)ccc2c1 ; c1ccc(-c2ccc(N(c3ccc4sc5ccccc5c4c3)c3cc4ccccc4c4ccccc34)c3oc4ccccc4c23)cc1]

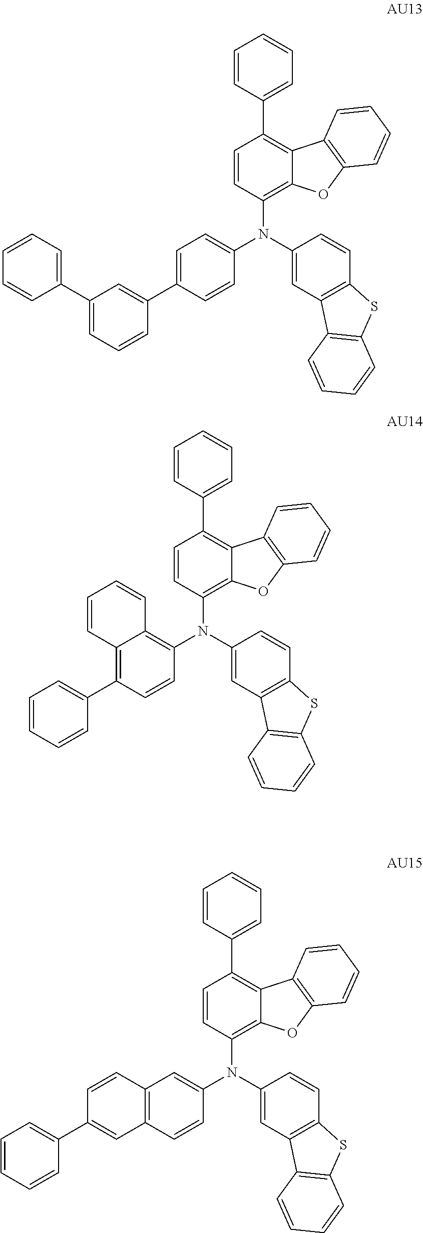 [SMILES: c1ccc(-c2cccc(-c3ccc(N(c4ccc5sc6ccccc6c5c4)c4ccc(-c5ccccc5)c5c4oc4ccccc45)cc3)c2)cc1 ; c1ccc(-c2ccc(N(c3ccc4sc5ccccc5c4c3)c3ccc(-c4ccccc4)c4c3oc3ccccc34)c3ccccc23)cc1 ; c1ccc(-c2ccc3cc(N(c4ccc5sc6ccccc6c5c4)c4ccc(-c5ccccc5)c5c4oc4ccccc45)ccc3c2)cc1]

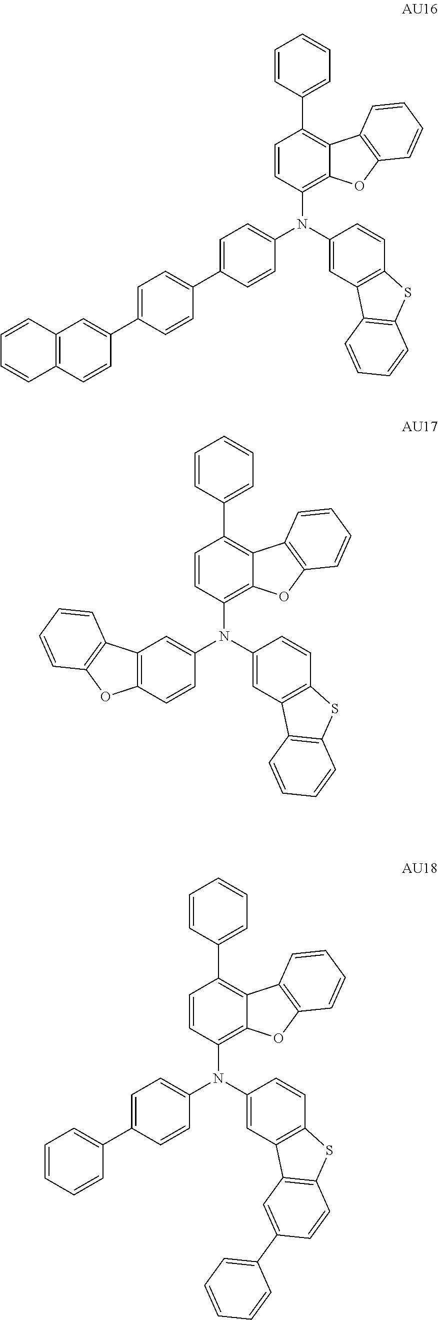 [SMILES: c1ccc(-c2ccc(N(c3ccc(-c4ccc(-c5ccc6ccccc6c5)cc4)cc3)c3ccc4sc5ccccc5c4c3)c3oc4ccccc4c23)cc1 ; c1ccc(-c2ccc(N(c3ccc4oc5ccccc5c4c3)c3ccc4sc5ccccc5c4c3)c3oc4ccccc4c23)cc1 ; c1ccc(-c2ccc(N(c3ccc4sc5ccc(-c6ccccc6)cc5c4c3)c3ccc(-c4ccccc4)c4c3oc3ccccc34)cc2)cc1]

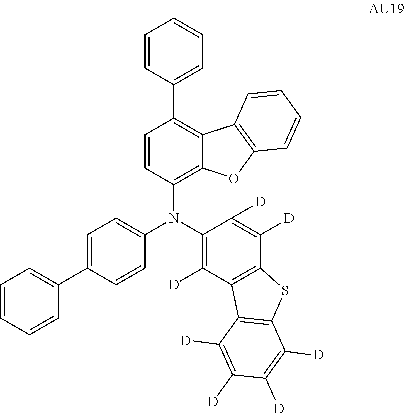 [SMILES: [2H]c1c([2H])c([2H])c2c(sc3c([2H])c([2H])c(N(c4ccc(-c5ccccc5)cc4)c4ccc(-c5ccccc5)c5c4oc4ccccc45)c([2H])c32)c1[2H]]

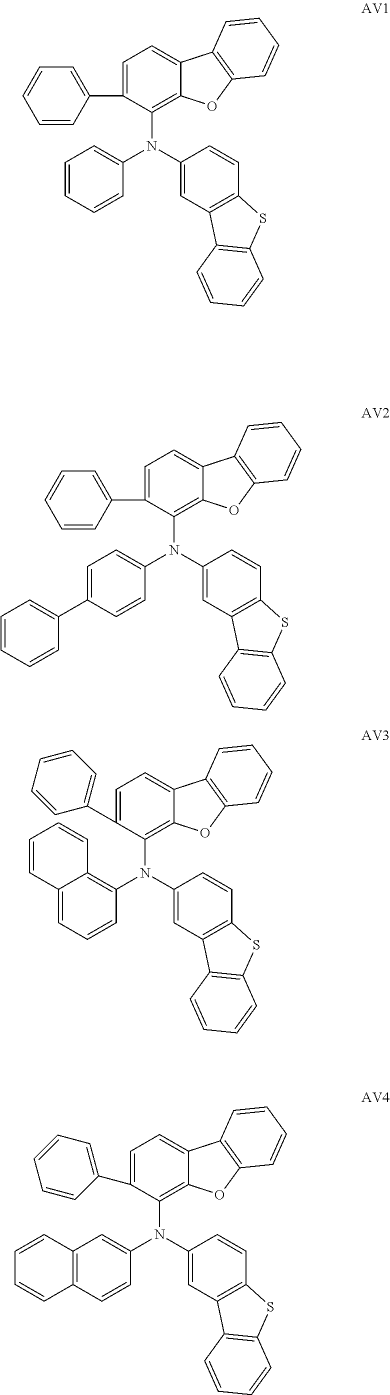

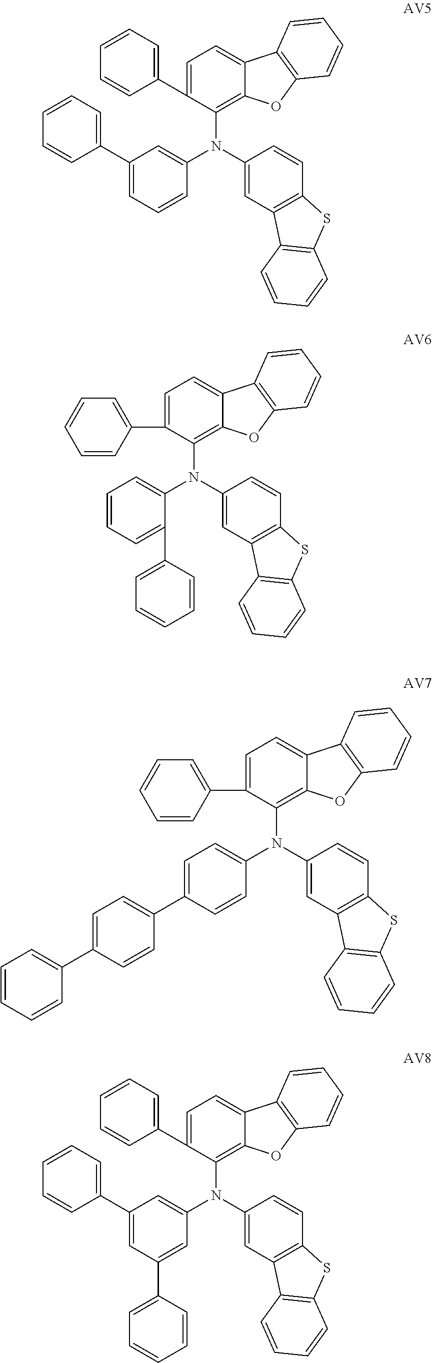

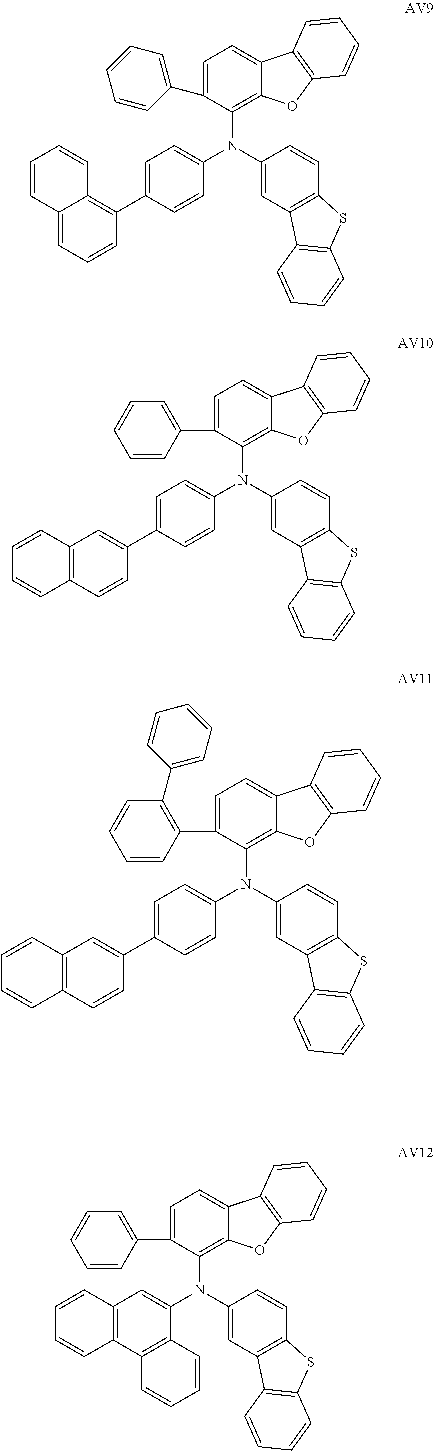 [SMILES: c1ccc(-c2ccc3c(oc4ccccc43)c2N(c2ccc(-c3cccc4ccccc34)cc2)c2ccc3sc4ccccc4c3c2)cc1 ; c1ccc(-c2ccc3c(oc4ccccc43)c2N(c2ccc(-c3ccc4ccccc4c3)cc2)c2ccc3sc4ccccc4c3c2)cc1 ; c1ccc(-c2ccccc2-c2ccc3c(oc4ccccc43)c2N(c2ccc(-c3ccc4ccccc4c3)cc2)c2ccc3sc4ccccc4c3c2)cc1 ; c1ccc(-c2ccc3c(oc4ccccc43)c2N(c2ccc3sc4ccccc4c3c2)c2cc3ccccc3c3ccccc23)cc1]

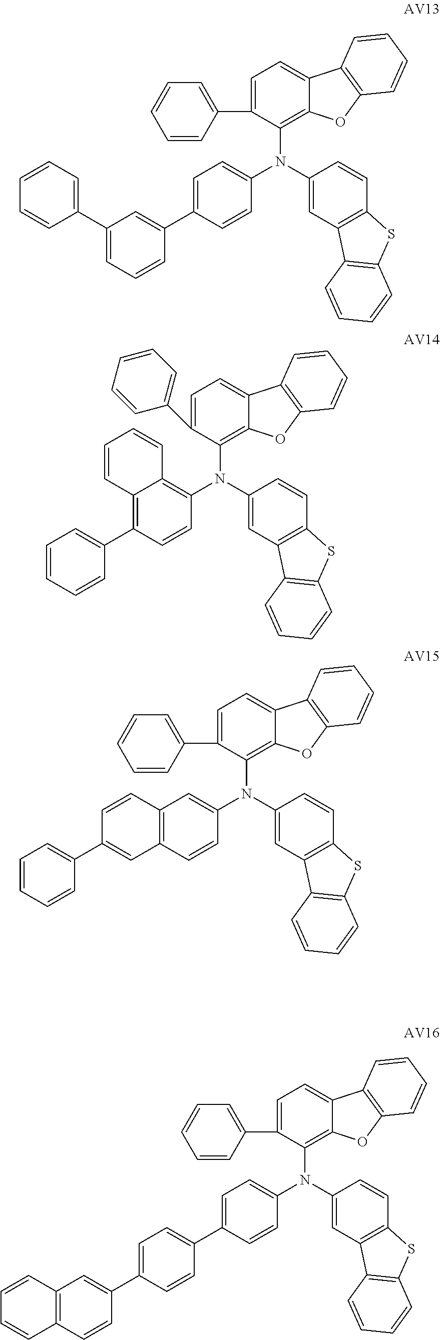

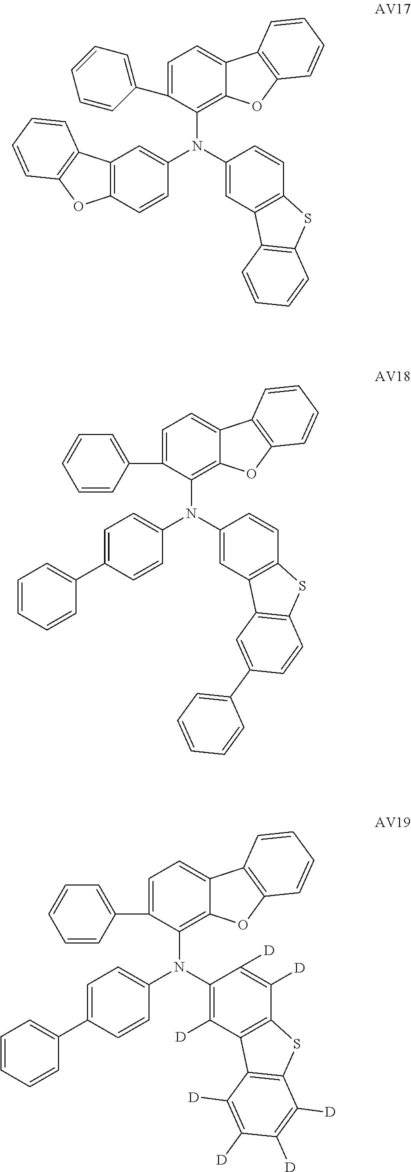 [SMILES: c1ccc(-c2ccc3c(oc4ccccc43)c2N(c2ccc3oc4ccccc4c3c2)c2ccc3sc4ccccc4c3c2)cc1 ; c1ccc(-c2ccc(N(c3ccc4sc5ccc(-c6ccccc6)cc5c4c3)c3c(-c4ccccc4)ccc4c3oc3ccccc34)cc2)cc1 ; [2H]c1c([2H])c([2H])c2c(sc3c([2H])c([2H])c(N(c4ccc(-c5ccccc5)cc4)c4c(-c5ccccc5)ccc5c4oc4ccccc45)c([2H])c32)c1[2H]]

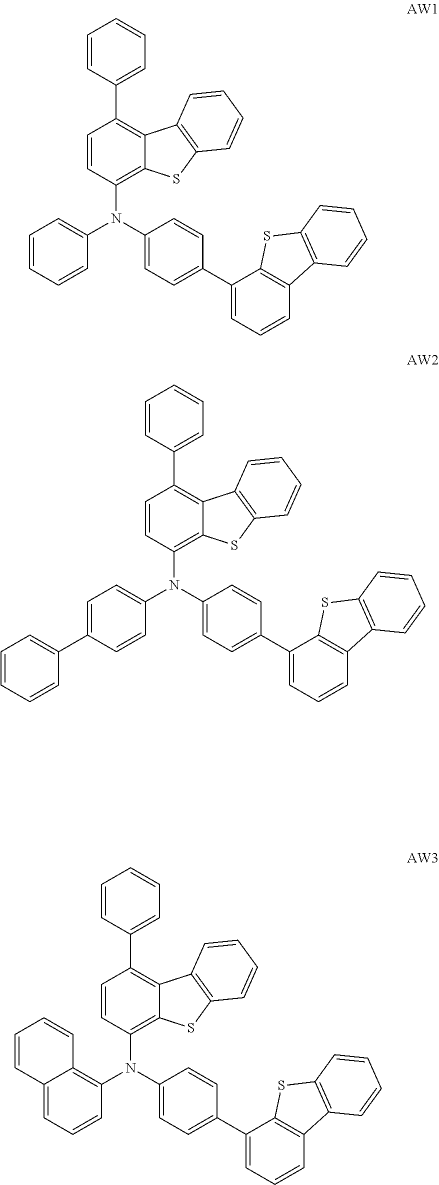 [SMILES: c1ccc(-c2ccc(N(c3ccccc3)c3ccc(-c4cccc5c4sc4ccccc45)cc3)c3sc4ccccc4c23)cc1 ; c1ccc(-c2ccc(N(c3ccc(-c4cccc5c4sc4ccccc45)cc3)c3ccc(-c4ccccc4)c4c3sc3ccccc34)cc2)cc1 ; c1ccc(-c2ccc(N(c3ccc(-c4cccc5c4sc4ccccc45)cc3)c3cccc4ccccc34)c3sc4ccccc4c23)cc1]

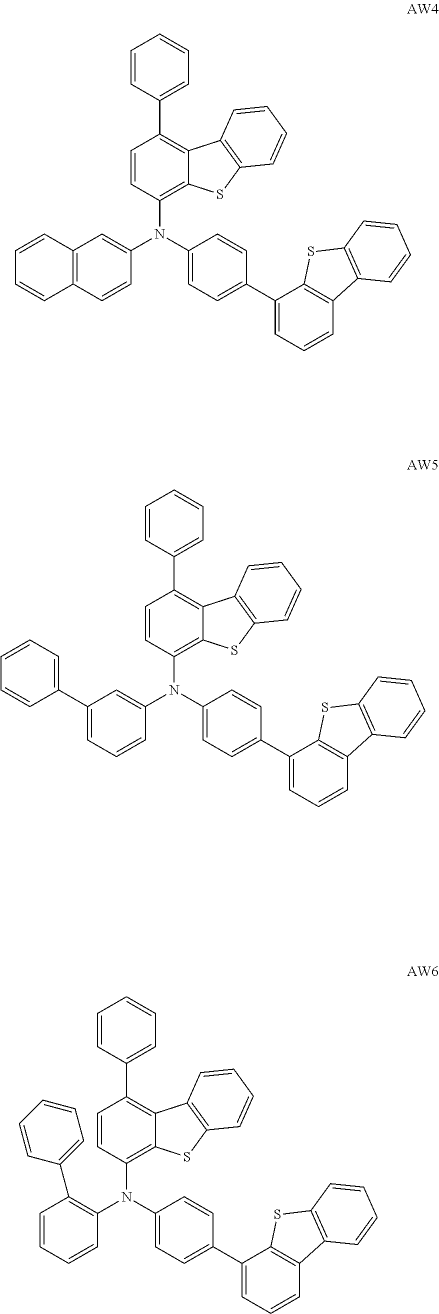 [SMILES: c1ccc(-c2ccc(N(c3ccc(-c4cccc5c4sc4ccccc45)cc3)c3ccc4ccccc4c3)c3sc4ccccc4c23)cc1 ; c1ccc(-c2cccc(N(c3ccc(-c4cccc5c4sc4ccccc45)cc3)c3ccc(-c4ccccc4)c4c3sc3ccccc34)c2)cc1 ; c1ccc(-c2ccccc2N(c2ccc(-c3cccc4c3sc3ccccc34)cc2)c2ccc(-c3ccccc3)c3c2sc2ccccc23)cc1]

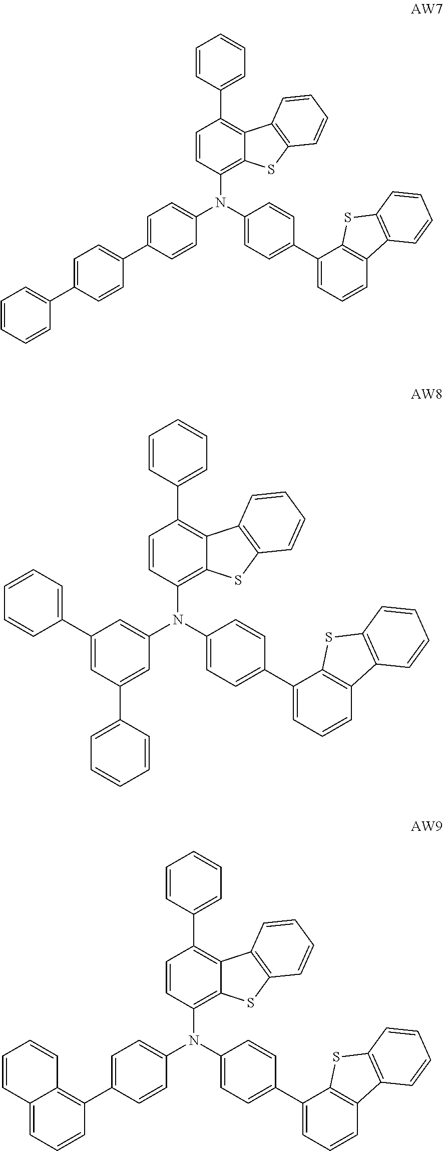 [SMILES: c1ccc(-c2ccc(-c3ccc(N(c4ccc(-c5cccc6c5sc5ccccc56)cc4)c4ccc(-c5ccccc5)c5c4sc4ccccc45)cc3)cc2)cc1 ; c1ccc(-c2cc(-c3ccccc3)cc(N(c3ccc(-c4cccc5c4sc4ccccc45)cc3)c3ccc(-c4ccccc4)c4c3sc3ccccc34)c2)cc1 ; c1ccc(-c2ccc(N(c3ccc(-c4cccc5ccccc45)cc3)c3ccc(-c4cccc5c4sc4ccccc45)cc3)c3sc4ccccc4c23)cc1]

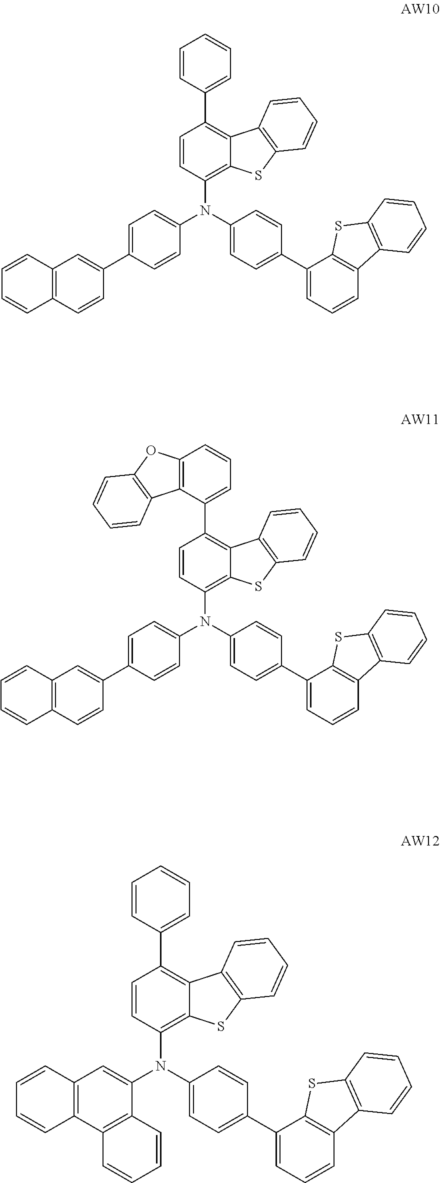 [SMILES: c1ccc(-c2ccc(N(c3ccc(-c4ccc5ccccc5c4)cc3)c3ccc(-c4cccc5c4sc4ccccc45)cc3)c3sc4ccccc4c23)cc1 ; c1ccc2cc(-c3ccc(N(c4ccc(-c5cccc6c5sc5ccccc56)cc4)c4ccc(-c5cccc6oc7ccccc7c56)c5c4sc4ccccc45)cc3)ccc2c1 ; c1ccc(-c2ccc(N(c3ccc(-c4cccc5c4sc4ccccc45)cc3)c3cc4ccccc4c4ccccc34)c3sc4ccccc4c23)cc1]

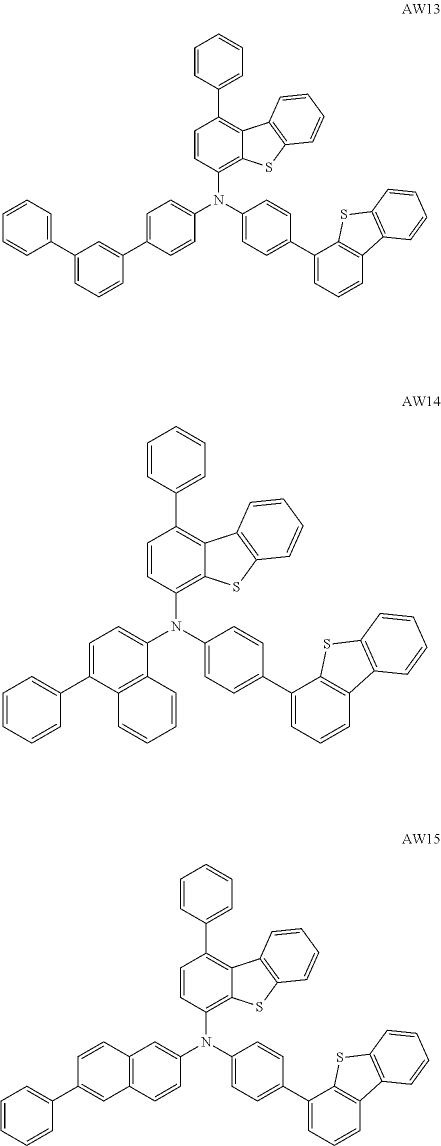 [SMILES: c1ccc(-c2cccc(-c3ccc(N(c4ccc(-c5cccc6c5sc5ccccc56)cc4)c4ccc(-c5ccccc5)c5c4sc4ccccc45)cc3)c2)cc1 ; c1ccc(-c2ccc(N(c3ccc(-c4cccc5c4sc4ccccc45)cc3)c3ccc(-c4ccccc4)c4c3sc3ccccc34)c3ccccc23)cc1 ; c1ccc(-c2ccc3cc(N(c4ccc(-c5cccc6c5sc5ccccc56)cc4)c4ccc(-c5ccccc5)c5c4sc4ccccc45)ccc3c2)cc1]

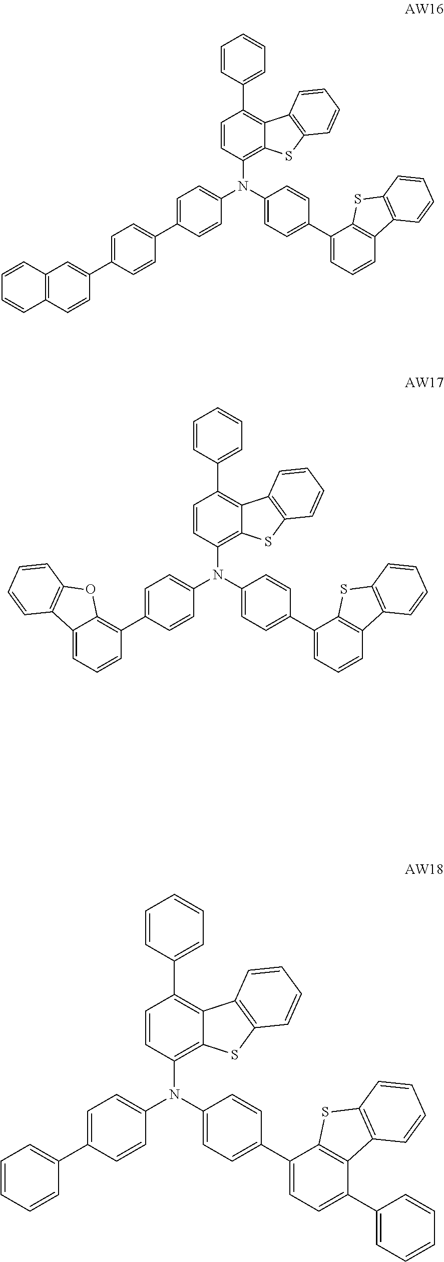 [SMILES: c1ccc(-c2ccc(N(c3ccc(-c4ccc(-c5ccc6ccccc6c5)cc4)cc3)c3ccc(-c4cccc5c4sc4ccccc45)cc3)c3sc4ccccc4c23)cc1 ; c1ccc(-c2ccc(N(c3ccc(-c4cccc5c4oc4ccccc45)cc3)c3ccc(-c4cccc5c4sc4ccccc45)cc3)c3sc4ccccc4c23)cc1 ; c1ccc(-c2ccc(N(c3ccc(-c4ccc(-c5ccccc5)c5c4sc4ccccc45)cc3)c3ccc(-c4ccccc4)c4c3sc3ccccc34)cc2)cc1]

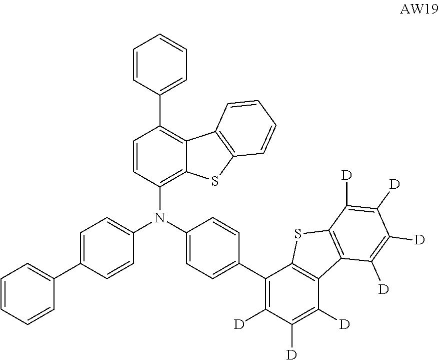 [SMILES: [2H]c1c([2H])c([2H])c2c(sc3c(-c4ccc(N(c5ccc(-c6ccccc6)cc5)c5ccc(-c6ccccc6)c6c5sc5ccccc56)cc4)c([2H])c([2H])c([2H])c32)c1[2H]]

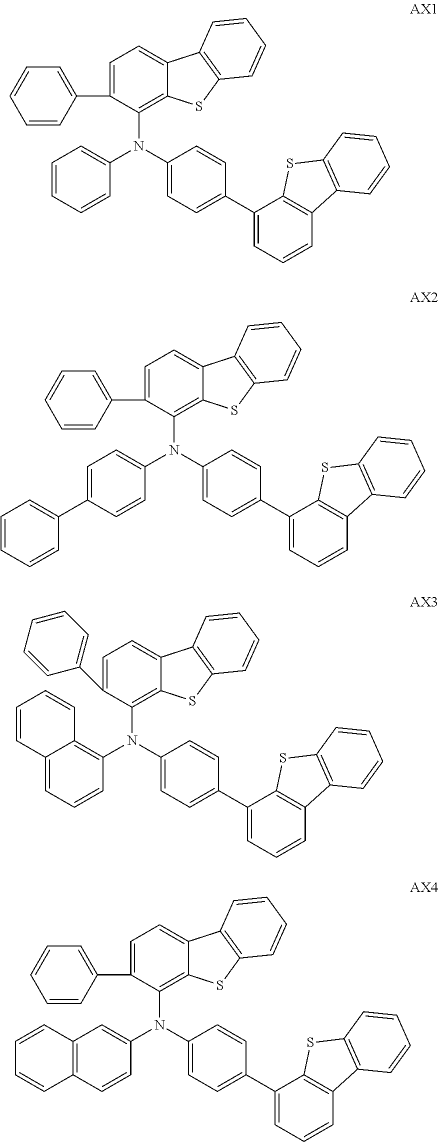

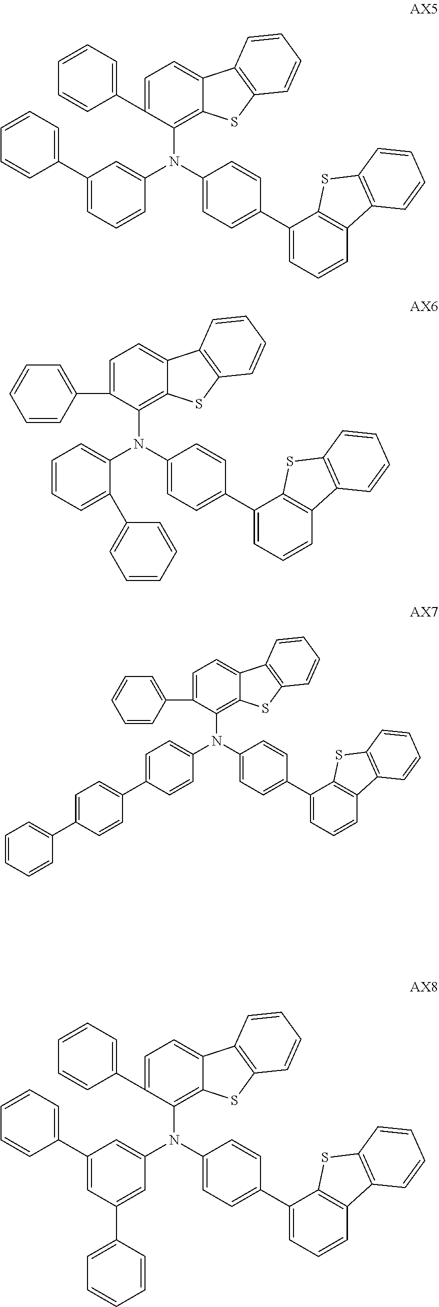 [SMILES: c1ccc(-c2cccc(N(c3ccc(-c4cccc5c4sc4ccccc45)cc3)c3c(-c4ccccc4)ccc4c3sc3ccccc34)c2)cc1 ; c1ccc(-c2ccccc2N(c2ccc(-c3cccc4c3sc3ccccc34)cc2)c2c(-c3ccccc3)ccc3c2sc2ccccc23)cc1 ; c1ccc(-c2ccc(-c3ccc(N(c4ccc(-c5cccc6c5sc5ccccc56)cc4)c4c(-c5ccccc5)ccc5c4sc4ccccc45)cc3)cc2)cc1 ; c1ccc(-c2cc(-c3ccccc3)cc(N(c3ccc(-c4cccc5c4sc4ccccc45)cc3)c3c(-c4ccccc4)ccc4c3sc3ccccc34)c2)cc1]

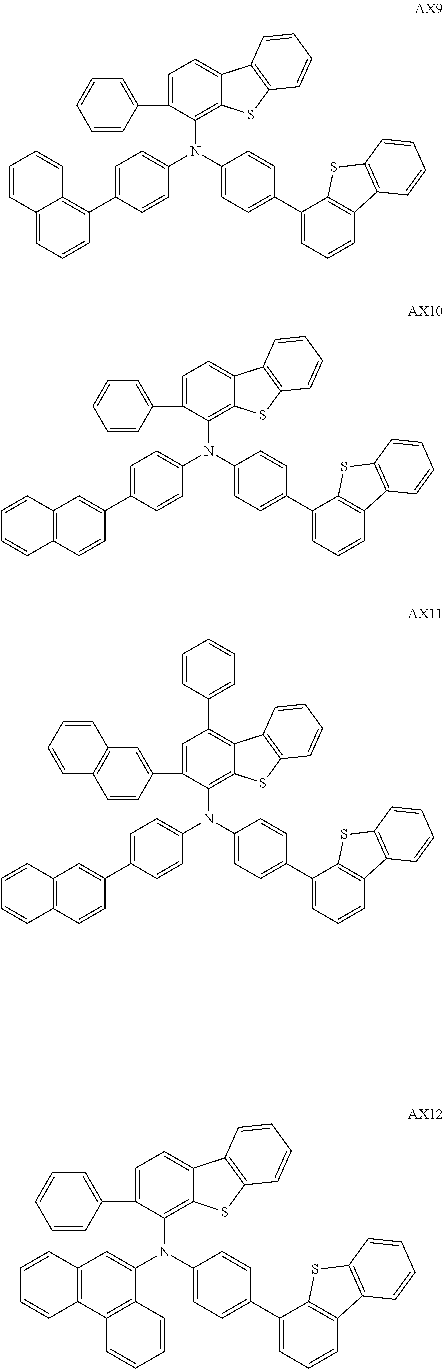 [SMILES: c1ccc(-c2ccc3c(sc4ccccc43)c2N(c2ccc(-c3cccc4ccccc34)cc2)c2ccc(-c3cccc4c3sc3ccccc34)cc2)cc1 ; c1ccc(-c2ccc3c(sc4ccccc43)c2N(c2ccc(-c3ccc4ccccc4c3)cc2)c2ccc(-c3cccc4c3sc3ccccc34)cc2)cc1 ; c1ccc(-c2cc(-c3ccc4ccccc4c3)c(N(c3ccc(-c4ccc5ccccc5c4)cc3)c3ccc(-c4cccc5c4sc4ccccc45)cc3)c3sc4ccccc4c23)cc1 ; c1ccc(-c2ccc3c(sc4ccccc43)c2N(c2ccc(-c3cccc4c3sc3ccccc34)cc2)c2cc3ccccc3c3ccccc23)cc1]

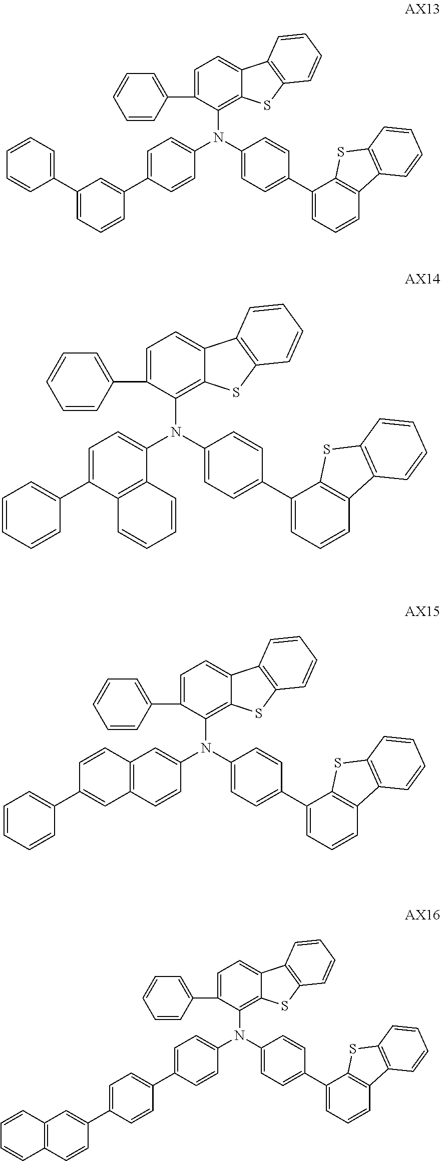 [SMILES: c1ccc(-c2cccc(-c3ccc(N(c4ccc(-c5cccc6c5sc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3cccc4c3sc3ccccc34)cc2)c2ccc(-c3ccccc3)c3ccccc23)cc1 ; c1ccc(-c2ccc3cc(N(c4ccc(-c5cccc6c5sc5ccccc56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3cccc4c3sc3ccccc34)cc2)cc1]

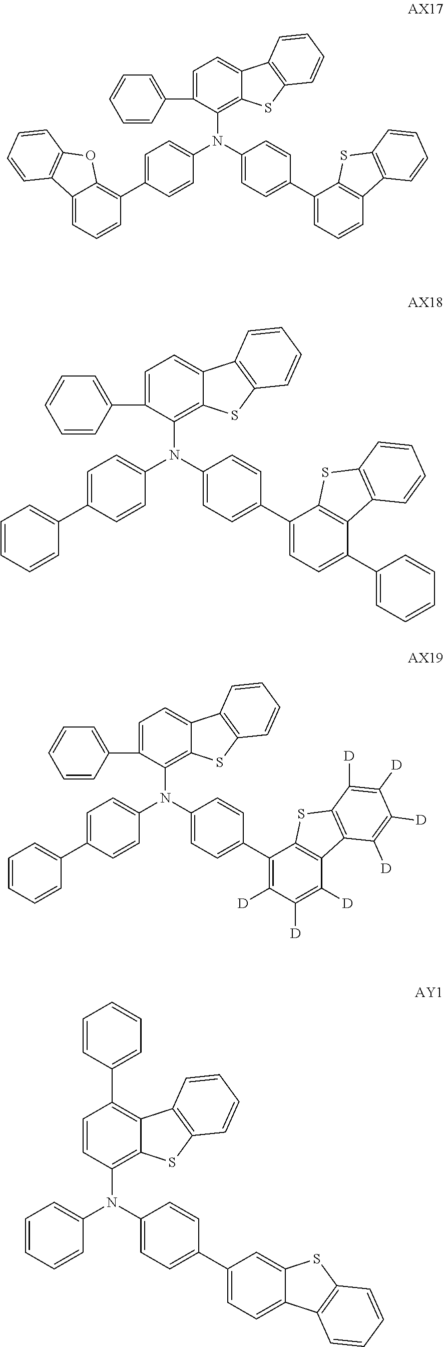 [SMILES: c1ccc(-c2ccc3c(sc4ccccc43)c2N(c2ccc(-c3cccc4c3oc3ccccc34)cc2)c2ccc(-c3cccc4c3sc3ccccc34)cc2)cc1 ; c1ccc(-c2ccc(N(c3ccc(-c4ccc(-c5ccccc5)c5c4sc4ccccc45)cc3)c3c(-c4ccccc4)ccc4c3sc3ccccc34)cc2)cc1 ; [2H]c1c([2H])c([2H])c2c(sc3c(-c4ccc(N(c5ccc(-c6ccccc6)cc5)c5c(-c6ccccc6)ccc6c5sc5ccccc56)cc4)c([2H])c([2H])c([2H])c32)c1[2H] ; c1ccc(-c2ccc(N(c3ccccc3)c3ccc(-c4ccc5c(c4)sc4ccccc45)cc3)c3sc4ccccc4c23)cc1]

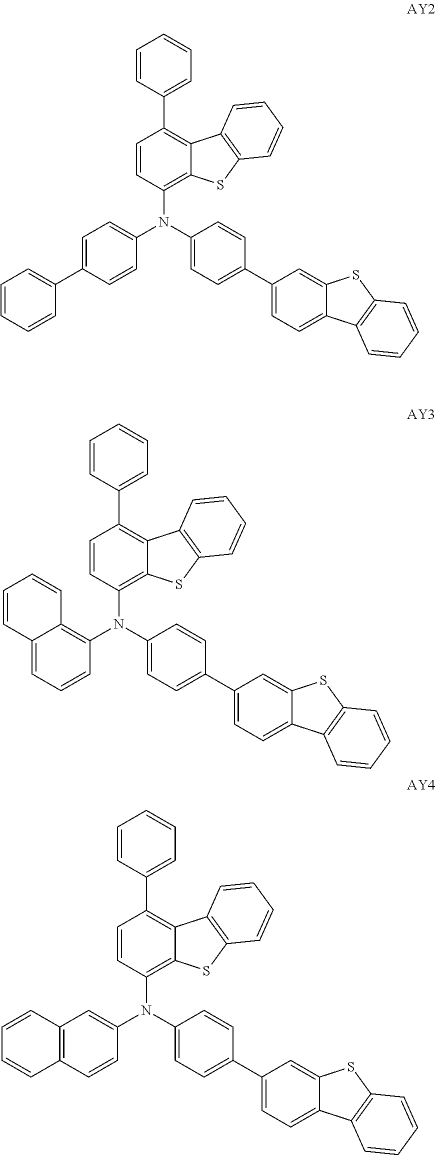 [SMILES: c1ccc(-c2ccc(N(c3ccc(-c4ccc5c(c4)sc4ccccc45)cc3)c3ccc(-c4ccccc4)c4c3sc3ccccc34)cc2)cc1 ; c1ccc(-c2ccc(N(c3ccc(-c4ccc5c(c4)sc4ccccc45)cc3)c3cccc4ccccc34)c3sc4ccccc4c23)cc1 ; c1ccc(-c2ccc(N(c3ccc(-c4ccc5c(c4)sc4ccccc45)cc3)c3ccc4ccccc4c3)c3sc4ccccc4c23)cc1]

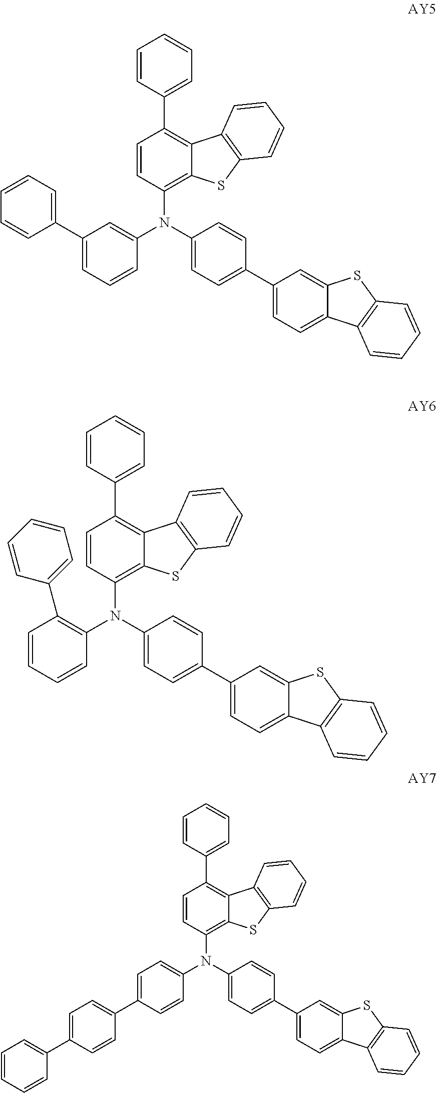 [SMILES: c1ccc(-c2cccc(N(c3ccc(-c4ccc5c(c4)sc4ccccc45)cc3)c3ccc(-c4ccccc4)c4c3sc3ccccc34)c2)cc1 ; c1ccc(-c2ccccc2N(c2ccc(-c3ccc4c(c3)sc3ccccc34)cc2)c2ccc(-c3ccccc3)c3c2sc2ccccc23)cc1 ; c1ccc(-c2ccc(-c3ccc(N(c4ccc(-c5ccc6c(c5)sc5ccccc56)cc4)c4ccc(-c5ccccc5)c5c4sc4ccccc45)cc3)cc2)cc1]

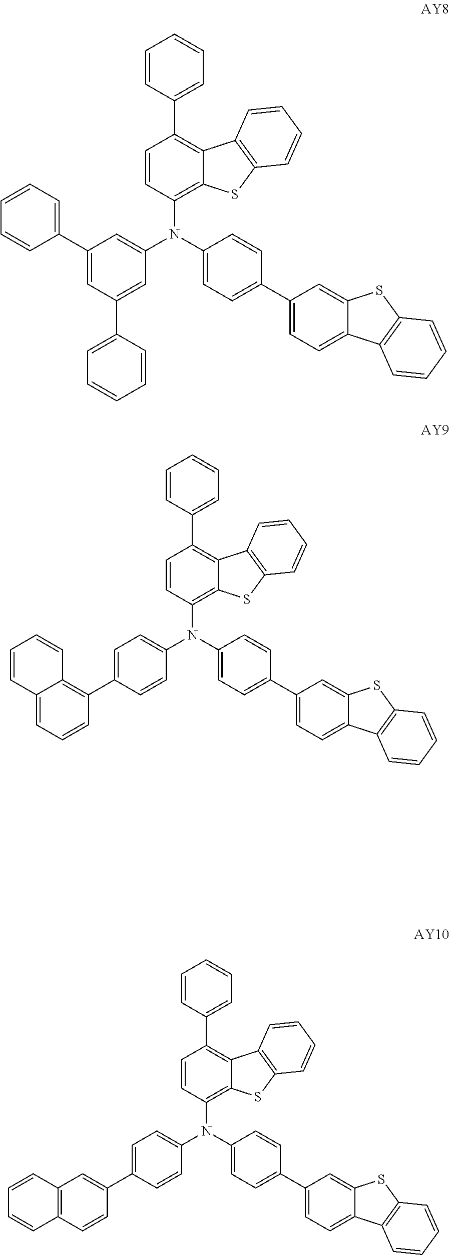 [SMILES: c1ccc(-c2cc(-c3ccccc3)cc(N(c3ccc(-c4ccc5c(c4)sc4ccccc45)cc3)c3ccc(-c4ccccc4)c4c3sc3ccccc34)c2)cc1 ; c1ccc(-c2ccc(N(c3ccc(-c4ccc5c(c4)sc4ccccc45)cc3)c3ccc(-c4cccc5ccccc45)cc3)c3sc4ccccc4c23)cc1 ; c1ccc(-c2ccc(N(c3ccc(-c4ccc5ccccc5c4)cc3)c3ccc(-c4ccc5c(c4)sc4ccccc45)cc3)c3sc4ccccc4c23)cc1]

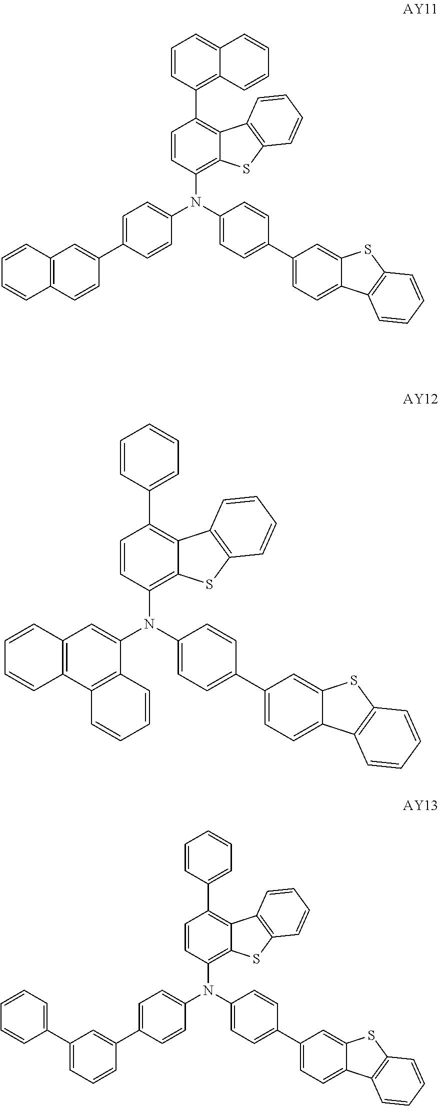 [SMILES: c1ccc2cc(-c3ccc(N(c4ccc(-c5ccc6c(c5)sc5ccccc56)cc4)c4ccc(-c5cccc6ccccc56)c5c4sc4ccccc45)cc3)ccc2c1 ; c1ccc(-c2ccc(N(c3ccc(-c4ccc5c(c4)sc4ccccc45)cc3)c3cc4ccccc4c4ccccc34)c3sc4ccccc4c23)cc1 ; c1ccc(-c2cccc(-c3ccc(N(c4ccc(-c5ccc6c(c5)sc5ccccc56)cc4)c4ccc(-c5ccccc5)c5c4sc4ccccc45)cc3)c2)cc1]

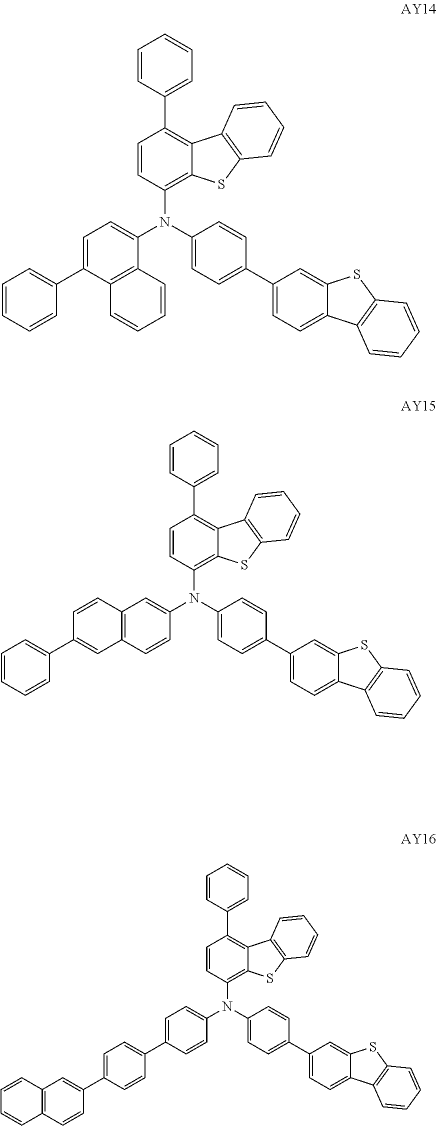 [SMILES: c1ccc(-c2ccc(N(c3ccc(-c4ccc5c(c4)sc4ccccc45)cc3)c3ccc(-c4ccccc4)c4c3sc3ccccc34)c3ccccc23)cc1 ; c1ccc(-c2ccc3cc(N(c4ccc(-c5ccc6c(c5)sc5ccccc56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4ccc5c(c4)sc4ccccc45)cc3)c3sc4ccccc4c23)cc1]

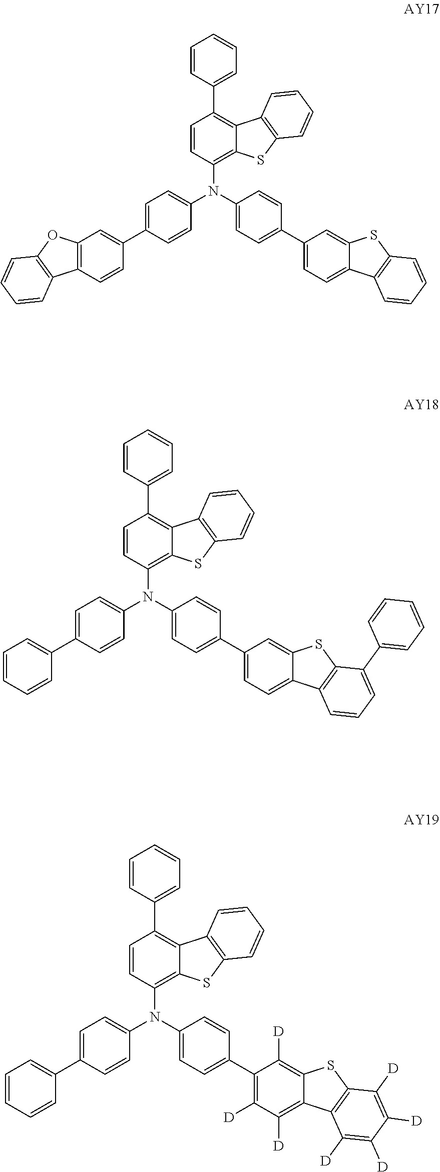 [SMILES: c1ccc(-c2ccc(N(c3ccc(-c4ccc5c(c4)oc4ccccc45)cc3)c3ccc(-c4ccc5c(c4)sc4ccccc45)cc3)c3sc4ccccc4c23)cc1 ; c1ccc(-c2ccc(N(c3ccc(-c4ccc5c(c4)sc4c(-c6ccccc6)cccc45)cc3)c3ccc(-c4ccccc4)c4c3sc3ccccc34)cc2)cc1 ; [2H]c1c([2H])c([2H])c2c(sc3c([2H])c(-c4ccc(N(c5ccc(-c6ccccc6)cc5)c5ccc(-c6ccccc6)c6c5sc5ccccc56)cc4)c([2H])c([2H])c32)c1[2H]]

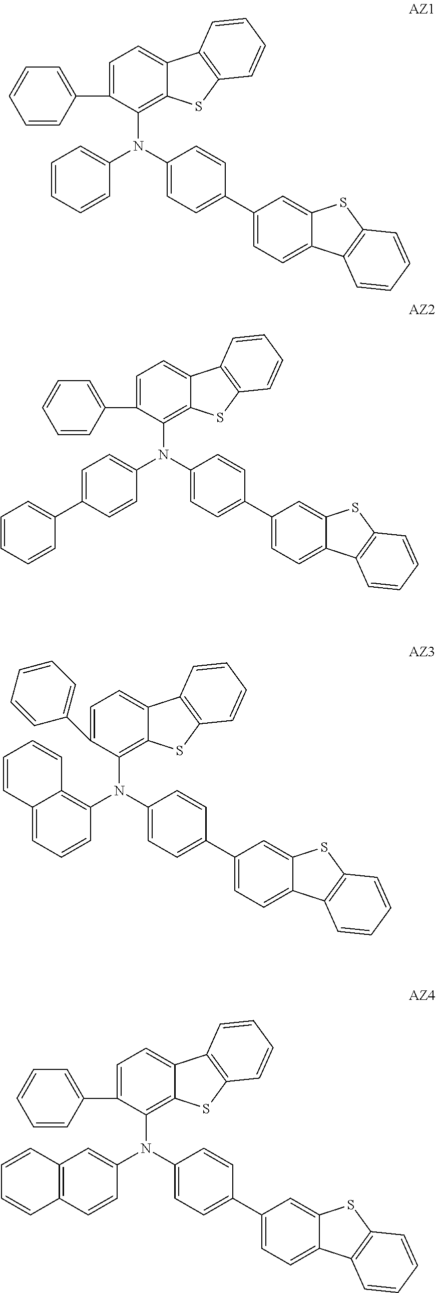 [SMILES: c1ccc(-c2ccc3c(sc4ccccc43)c2N(c2ccccc2)c2ccc(-c3ccc4c(c3)sc3ccccc34)cc2)cc1 ; c1ccc(-c2ccc(N(c3ccc(-c4ccc5c(c4)sc4ccccc45)cc3)c3c(-c4ccccc4)ccc4c3sc3ccccc34)cc2)cc1 ; c1ccc(-c2ccc3c(sc4ccccc43)c2N(c2ccc(-c3ccc4c(c3)sc3ccccc34)cc2)c2cccc3ccccc23)cc1 ; c1ccc(-c2ccc3c(sc4ccccc43)c2N(c2ccc(-c3ccc4c(c3)sc3ccccc34)cc2)c2ccc3ccccc3c2)cc1]

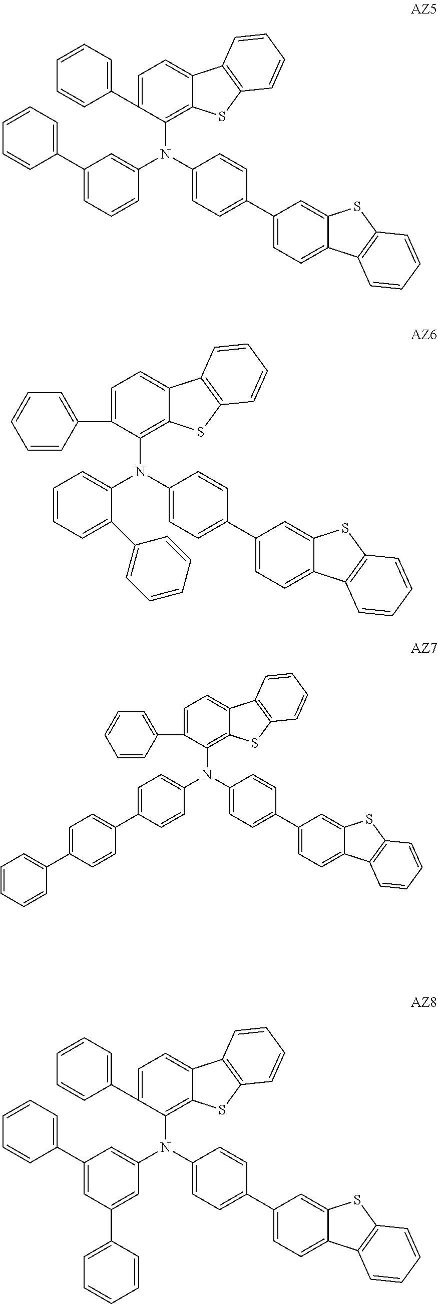 [SMILES: c1ccc(-c2cccc(N(c3ccc(-c4ccc5c(c4)sc4ccccc45)cc3)c3c(-c4ccccc4)ccc4c3sc3ccccc34)c2)cc1 ; c1ccc(-c2ccccc2N(c2ccc(-c3ccc4c(c3)sc3ccccc34)cc2)c2c(-c3ccccc3)ccc3c2sc2ccccc23)cc1 ; c1ccc(-c2ccc(-c3ccc(N(c4ccc(-c5ccc6c(c5)sc5ccccc56)cc4)c4c(-c5ccccc5)ccc5c4sc4ccccc45)cc3)cc2)cc1 ; c1ccc(-c2cc(-c3ccccc3)cc(N(c3ccc(-c4ccc5c(c4)sc4ccccc45)cc3)c3c(-c4ccccc4)ccc4c3sc3ccccc34)c2)cc1]

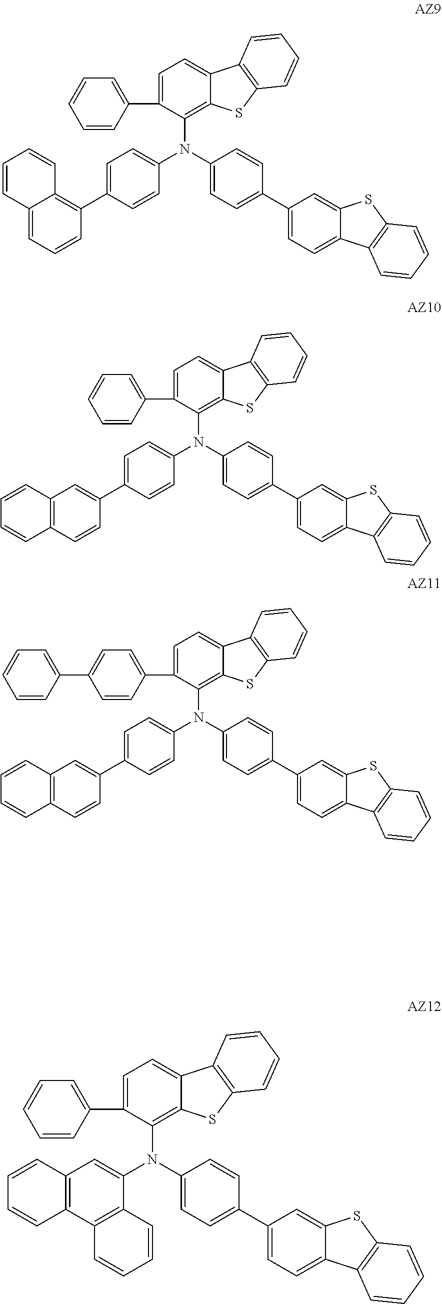 [SMILES: c1ccc(-c2ccc3c(sc4ccccc43)c2N(c2ccc(-c3ccc4c(c3)sc3ccccc34)cc2)c2ccc(-c3cccc4ccccc34)cc2)cc1 ; c1ccc(-c2ccc3c(sc4ccccc43)c2N(c2ccc(-c3ccc4ccccc4c3)cc2)c2ccc(-c3ccc4c(c3)sc3ccccc34)cc2)cc1 ; c1ccc(-c2ccc(-c3ccc4c(sc5ccccc54)c3N(c3ccc(-c4ccc5ccccc5c4)cc3)c3ccc(-c4ccc5c(c4)sc4ccccc45)cc3)cc2)cc1 ; c1ccc(-c2ccc3c(sc4ccccc43)c2N(c2ccc(-c3ccc4c(c3)sc3ccccc34)cc2)c2cc3ccccc3c3ccccc23)cc1]

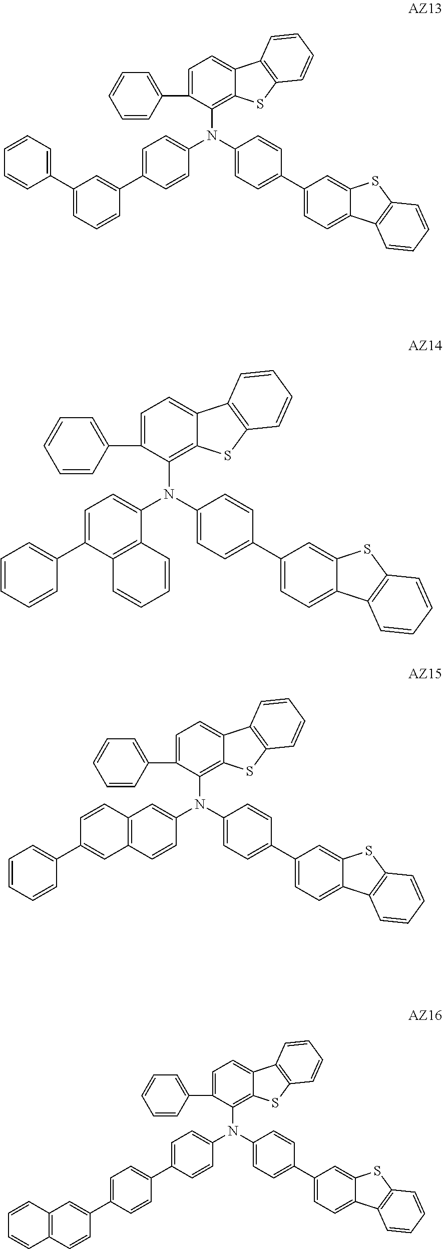 [SMILES: c1ccc(-c2cccc(-c3ccc(N(c4ccc(-c5ccc6c(c5)sc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3ccc4c(c3)sc3ccccc34)cc2)c2ccc(-c3ccccc3)c3ccccc23)cc1 ; c1ccc(-c2ccc3cc(N(c4ccc(-c5ccc6c(c5)sc5ccccc56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3ccc4c(c3)sc3ccccc34)cc2)cc1]

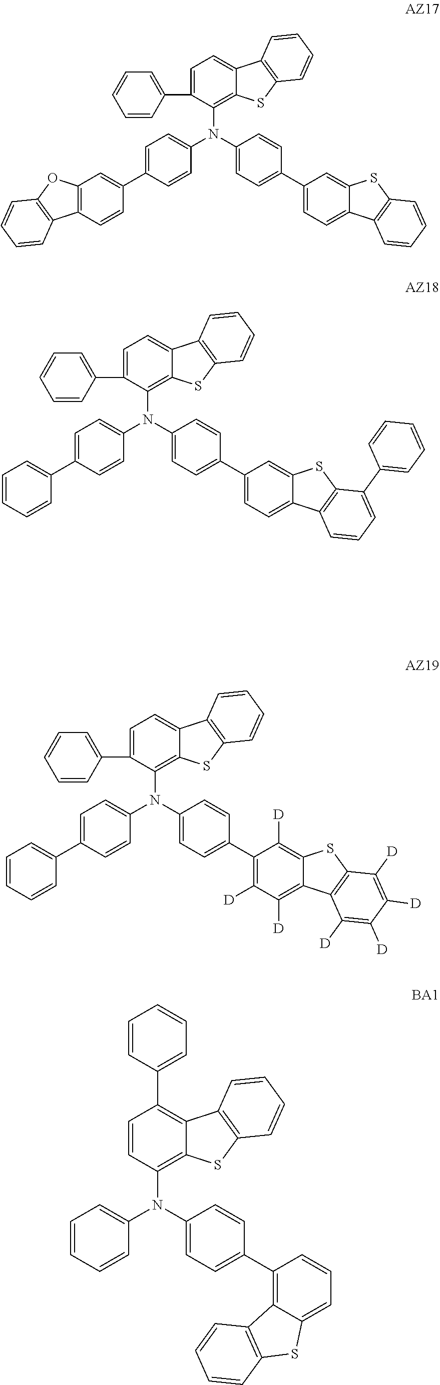 [SMILES: c1ccc(-c2ccc3c(sc4ccccc43)c2N(c2ccc(-c3ccc4c(c3)oc3ccccc34)cc2)c2ccc(-c3ccc4c(c3)sc3ccccc34)cc2)cc1 ; c1ccc(-c2ccc(N(c3ccc(-c4ccc5c(c4)sc4c(-c6ccccc6)cccc45)cc3)c3c(-c4ccccc4)ccc4c3sc3ccccc34)cc2)cc1 ; [2H]c1c([2H])c([2H])c2c(sc3c([2H])c(-c4ccc(N(c5ccc(-c6ccccc6)cc5)c5c(-c6ccccc6)ccc6c5sc5ccccc56)cc4)c([2H])c([2H])c32)c1[2H] ; c1ccc(-c2ccc(N(c3ccccc3)c3ccc(-c4cccc5sc6ccccc6c45)cc3)c3sc4ccccc4c23)cc1]

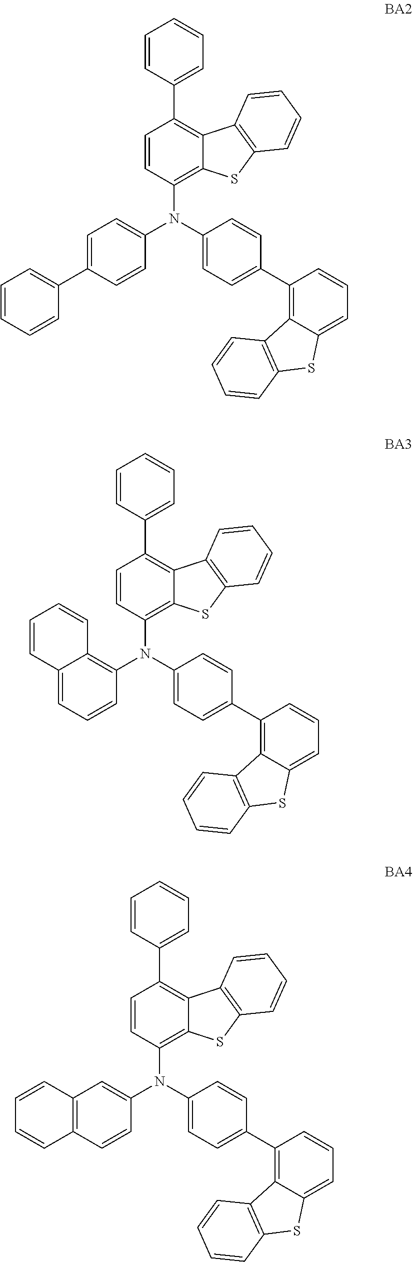 [SMILES: c1ccc(-c2ccc(N(c3ccc(-c4cccc5sc6ccccc6c45)cc3)c3ccc(-c4ccccc4)c4c3sc3ccccc34)cc2)cc1 ; c1ccc(-c2ccc(N(c3ccc(-c4cccc5sc6ccccc6c45)cc3)c3cccc4ccccc34)c3sc4ccccc4c23)cc1 ; c1ccc(-c2ccc(N(c3ccc(-c4cccc5sc6ccccc6c45)cc3)c3ccc4ccccc4c3)c3sc4ccccc4c23)cc1]

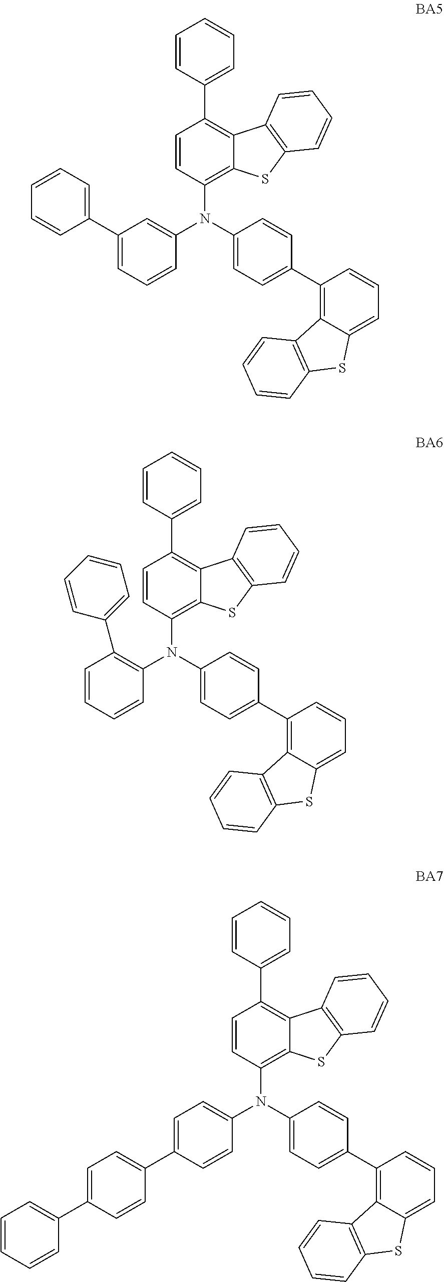 [SMILES: c1ccc(-c2cccc(N(c3ccc(-c4cccc5sc6ccccc6c45)cc3)c3ccc(-c4ccccc4)c4c3sc3ccccc34)c2)cc1 ; c1ccc(-c2ccccc2N(c2ccc(-c3cccc4sc5ccccc5c34)cc2)c2ccc(-c3ccccc3)c3c2sc2ccccc23)cc1 ; c1ccc(-c2ccc(-c3ccc(N(c4ccc(-c5cccc6sc7ccccc7c56)cc4)c4ccc(-c5ccccc5)c5c4sc4ccccc45)cc3)cc2)cc1]

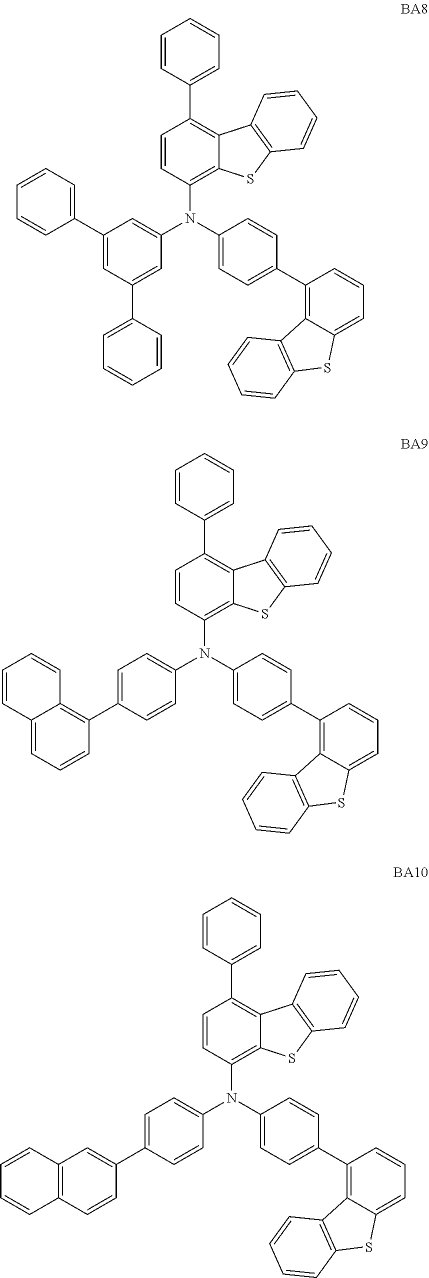 [SMILES: c1ccc(-c2cc(-c3ccccc3)cc(N(c3ccc(-c4cccc5sc6ccccc6c45)cc3)c3ccc(-c4ccccc4)c4c3sc3ccccc34)c2)cc1 ; c1ccc(-c2ccc(N(c3ccc(-c4cccc5ccccc45)cc3)c3ccc(-c4cccc5sc6ccccc6c45)cc3)c3sc4ccccc4c23)cc1 ; c1ccc(-c2ccc(N(c3ccc(-c4ccc5ccccc5c4)cc3)c3ccc(-c4cccc5sc6ccccc6c45)cc3)c3sc4ccccc4c23)cc1]

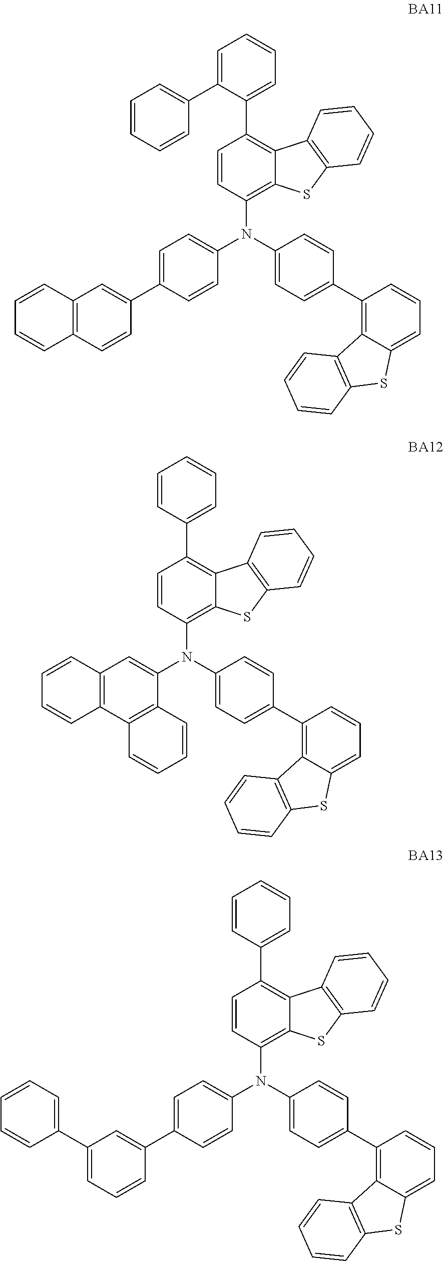

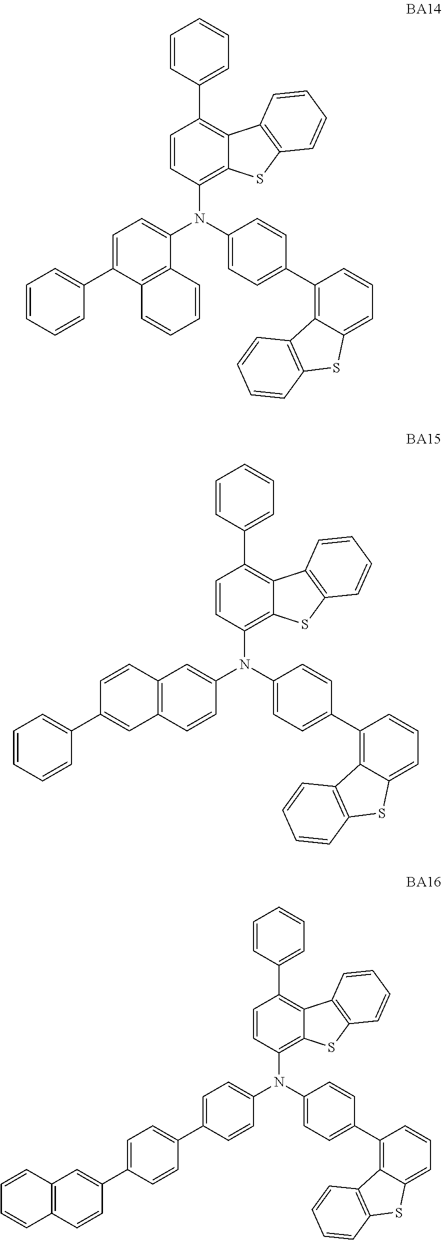 [SMILES: c1ccc(-c2ccc(N(c3ccc(-c4cccc5sc6ccccc6c45)cc3)c3ccc(-c4ccccc4)c4c3sc3ccccc34)c3ccccc23)cc1 ; c1ccc(-c2ccc3cc(N(c4ccc(-c5cccc6sc7ccccc7c56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4cccc5sc6ccccc6c45)cc3)c3sc4ccccc4c23)cc1]

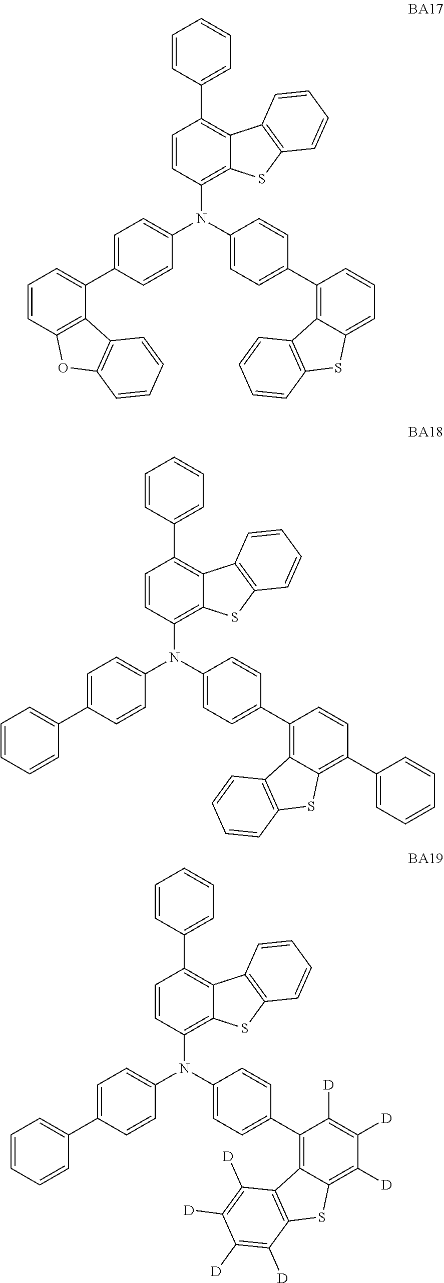 [SMILES: c1ccc(-c2ccc(N(c3ccc(-c4cccc5oc6ccccc6c45)cc3)c3ccc(-c4cccc5sc6ccccc6c45)cc3)c3sc4ccccc4c23)cc1 ; c1ccc(-c2ccc(N(c3ccc(-c4ccc(-c5ccccc5)c5sc6ccccc6c45)cc3)c3ccc(-c4ccccc4)c4c3sc3ccccc34)cc2)cc1 ; [2H]c1c([2H])c([2H])c2c(sc3c([2H])c([2H])c([2H])c(-c4ccc(N(c5ccc(-c6ccccc6)cc5)c5ccc(-c6ccccc6)c6c5sc5ccccc56)cc4)c32)c1[2H]]

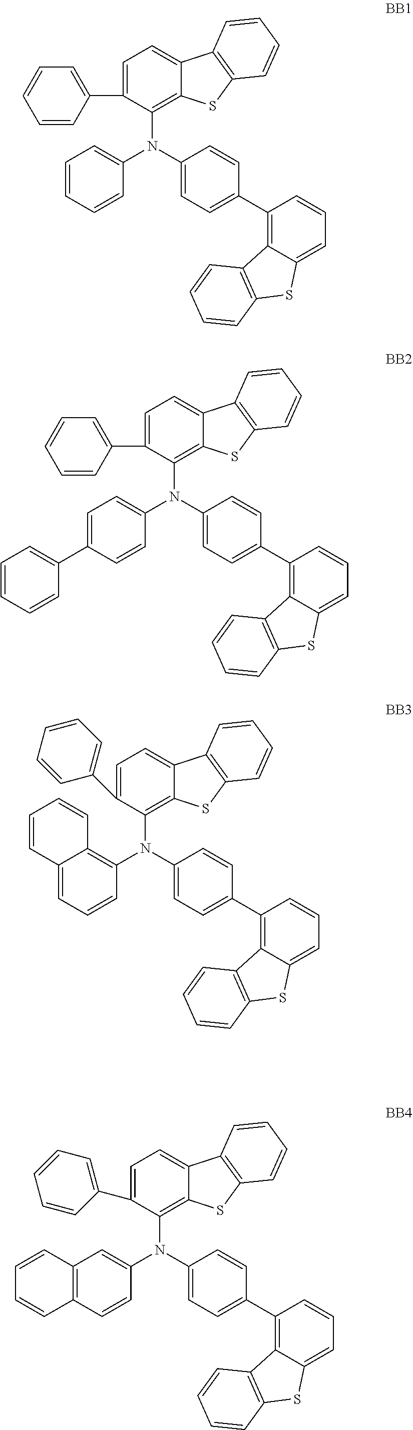

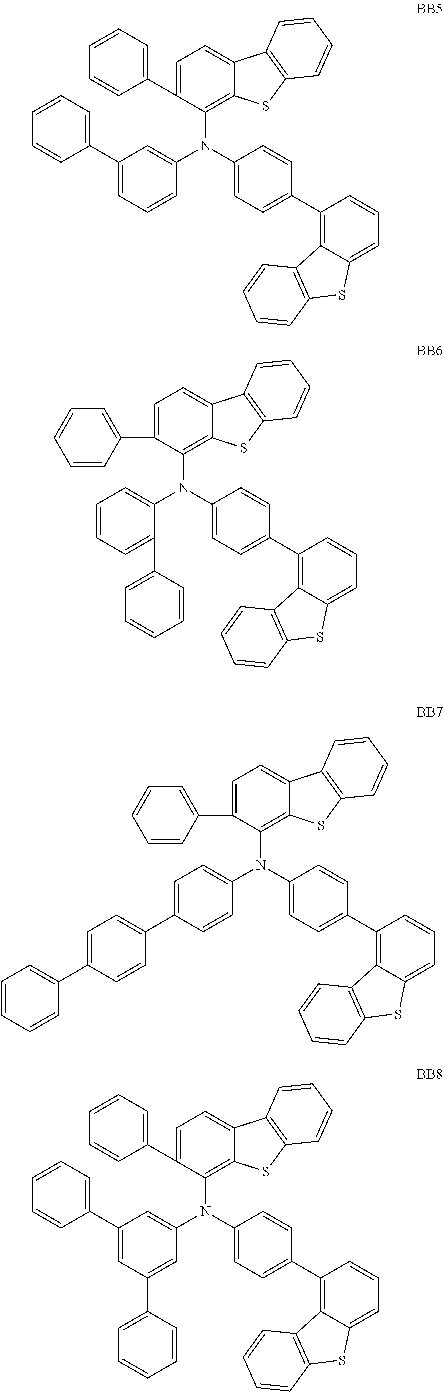

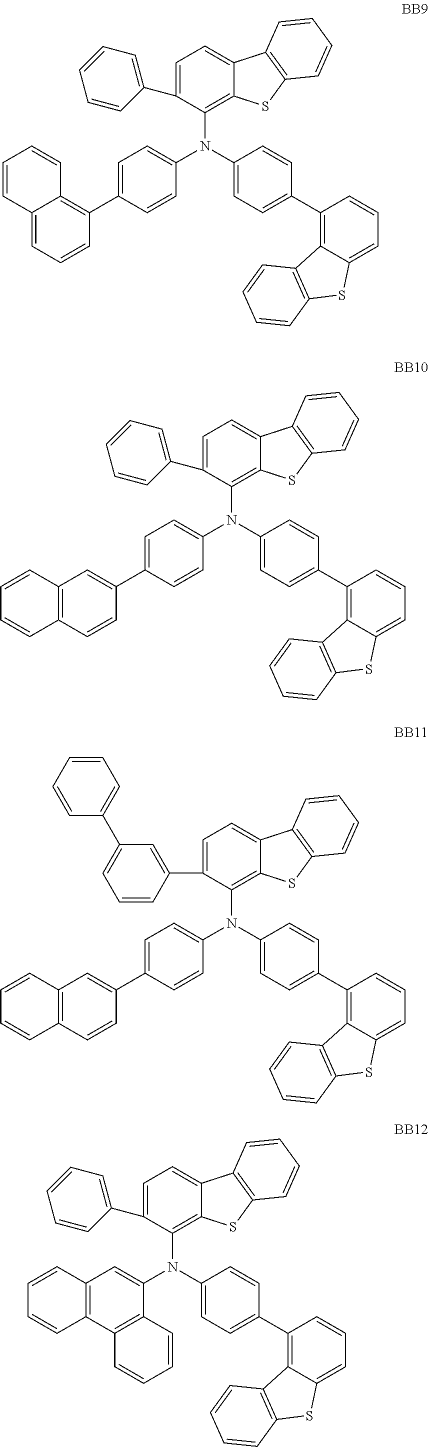

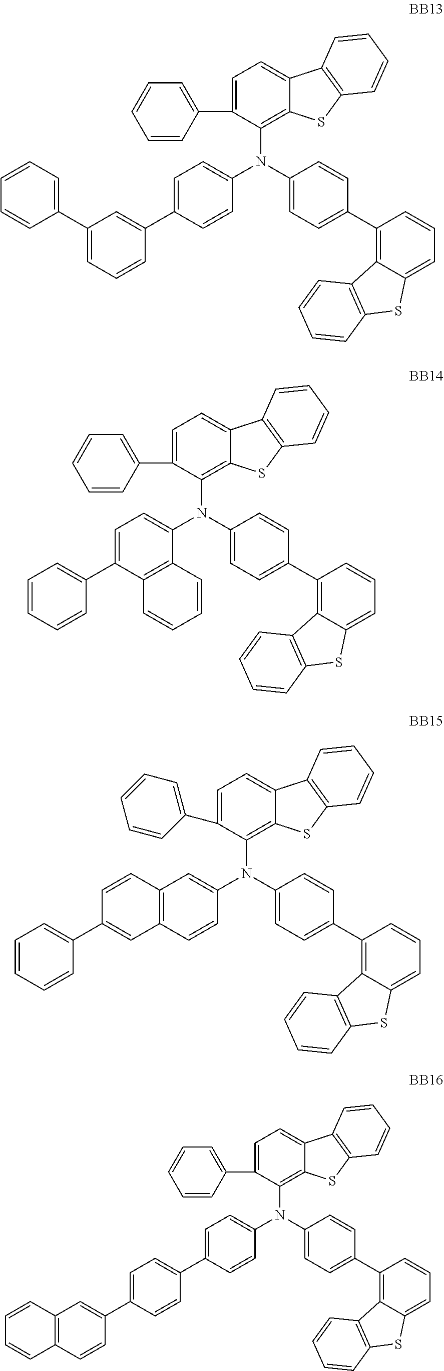 [SMILES: c1ccc(-c2cccc(-c3ccc(N(c4ccc(-c5cccc6sc7ccccc7c56)cc4)c4c(-c5ccccc5)ccc5c4sc4ccccc45)cc3)c2)cc1 ; c1ccc(-c2ccc3c(sc4ccccc43)c2N(c2ccc(-c3cccc4sc5ccccc5c34)cc2)c2ccc(-c3ccccc3)c3ccccc23)cc1 ; c1ccc(-c2ccc3cc(N(c4ccc(-c5cccc6sc7ccccc7c56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3cccc4sc5ccccc5c34)cc2)cc1]

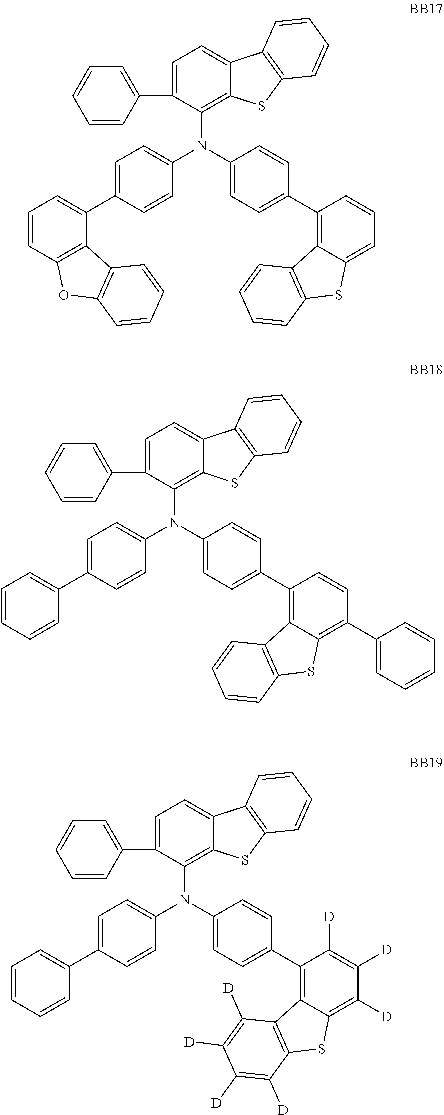 [SMILES: c1ccc(-c2ccc3c(sc4ccccc43)c2N(c2ccc(-c3cccc4oc5ccccc5c34)cc2)c2ccc(-c3cccc4sc5ccccc5c34)cc2)cc1 ; c1ccc(-c2ccc(N(c3ccc(-c4ccc(-c5ccccc5)c5sc6ccccc6c45)cc3)c3c(-c4ccccc4)ccc4c3sc3ccccc34)cc2)cc1 ; [2H]c1c([2H])c([2H])c2c(sc3c([2H])c([2H])c([2H])c(-c4ccc(N(c5ccc(-c6ccccc6)cc5)c5c(-c6ccccc6)ccc6c5sc5ccccc56)cc4)c32)c1[2H]]

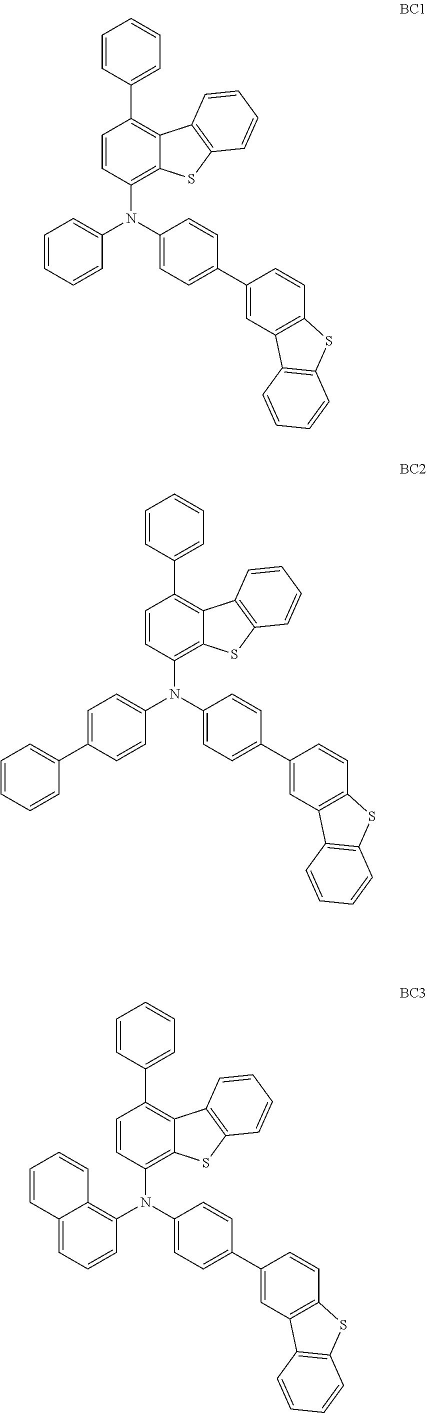 [SMILES: c1ccc(-c2ccc(N(c3ccccc3)c3ccc(-c4ccc5sc6ccccc6c5c4)cc3)c3sc4ccccc4c23)cc1 ; c1ccc(-c2ccc(N(c3ccc(-c4ccc5sc6ccccc6c5c4)cc3)c3ccc(-c4ccccc4)c4c3sc3ccccc34)cc2)cc1 ; c1ccc(-c2ccc(N(c3ccc(-c4ccc5sc6ccccc6c5c4)cc3)c3cccc4ccccc34)c3sc4ccccc4c23)cc1]

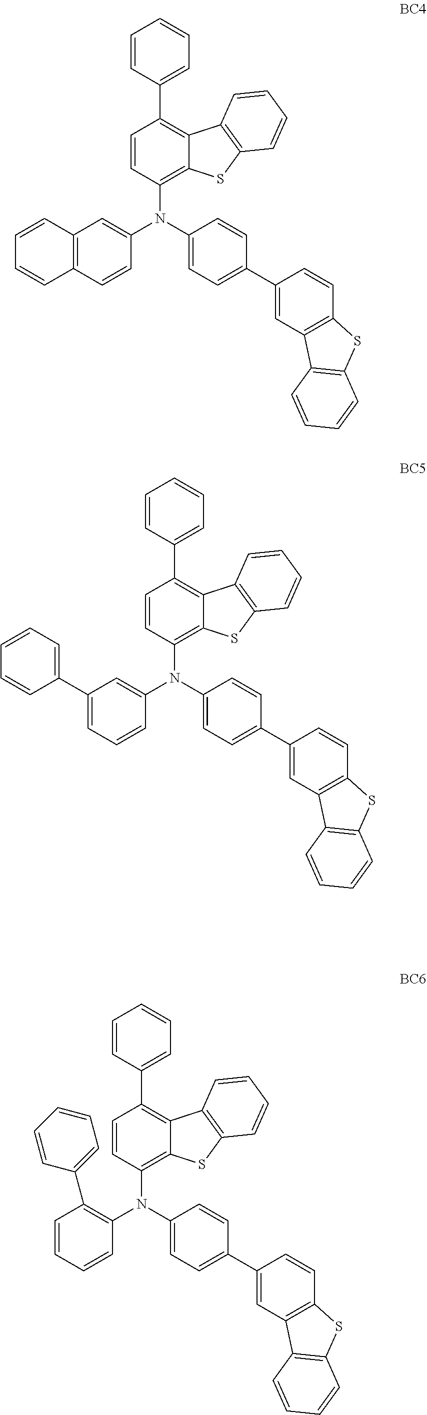 [SMILES: c1ccc(-c2ccc(N(c3ccc(-c4ccc5sc6ccccc6c5c4)cc3)c3ccc4ccccc4c3)c3sc4ccccc4c23)cc1 ; c1ccc(-c2cccc(N(c3ccc(-c4ccc5sc6ccccc6c5c4)cc3)c3ccc(-c4ccccc4)c4c3sc3ccccc34)c2)cc1 ; c1ccc(-c2ccccc2N(c2ccc(-c3ccc4sc5ccccc5c4c3)cc2)c2ccc(-c3ccccc3)c3c2sc2ccccc23)cc1]

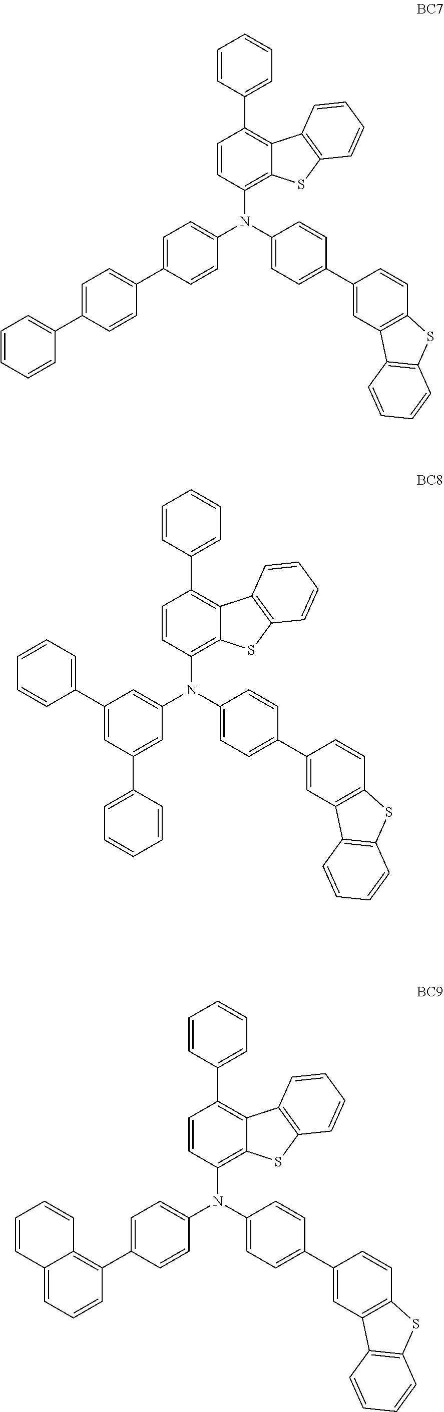 [SMILES: c1ccc(-c2ccc(-c3ccc(N(c4ccc(-c5ccc6sc7ccccc7c6c5)cc4)c4ccc(-c5ccccc5)c5c4sc4ccccc45)cc3)cc2)cc1 ; c1ccc(-c2cc(-c3ccccc3)cc(N(c3ccc(-c4ccc5sc6ccccc6c5c4)cc3)c3ccc(-c4ccccc4)c4c3sc3ccccc34)c2)cc1 ; c1ccc(-c2ccc(N(c3ccc(-c4ccc5sc6ccccc6c5c4)cc3)c3ccc(-c4cccc5ccccc45)cc3)c3sc4ccccc4c23)cc1]

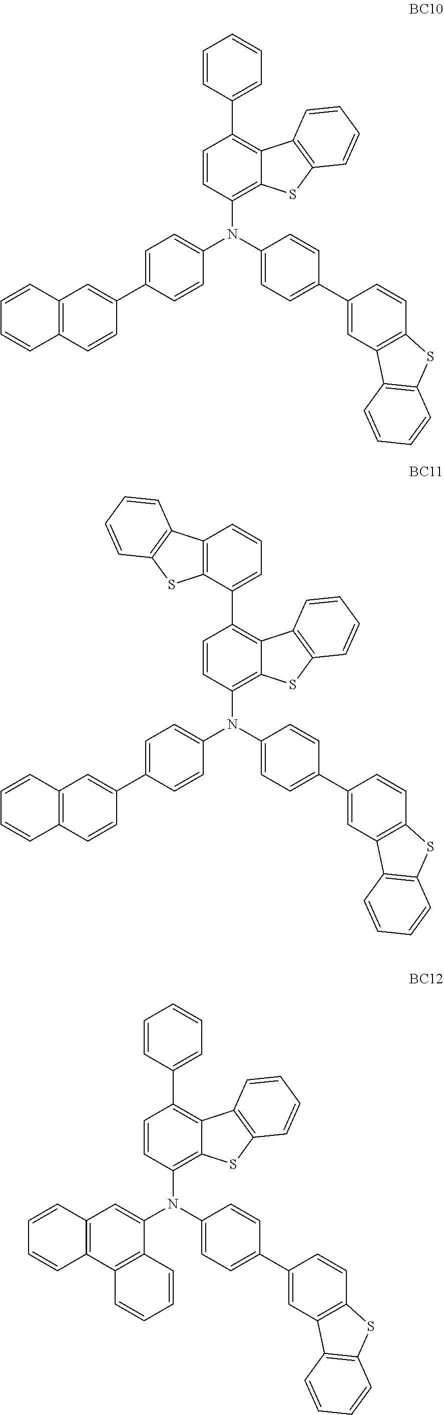 [SMILES: c1ccc(-c2ccc(N(c3ccc(-c4ccc5ccccc5c4)cc3)c3ccc(-c4ccc5sc6ccccc6c5c4)cc3)c3sc4ccccc4c23)cc1 ; c1ccc2cc(-c3ccc(N(c4ccc(-c5ccc6sc7ccccc7c6c5)cc4)c4ccc(-c5cccc6c5sc5ccccc56)c5c4sc4ccccc45)cc3)ccc2c1 ; c1ccc(-c2ccc(N(c3ccc(-c4ccc5sc6ccccc6c5c4)cc3)c3cc4ccccc4c4ccccc34)c3sc4ccccc4c23)cc1]

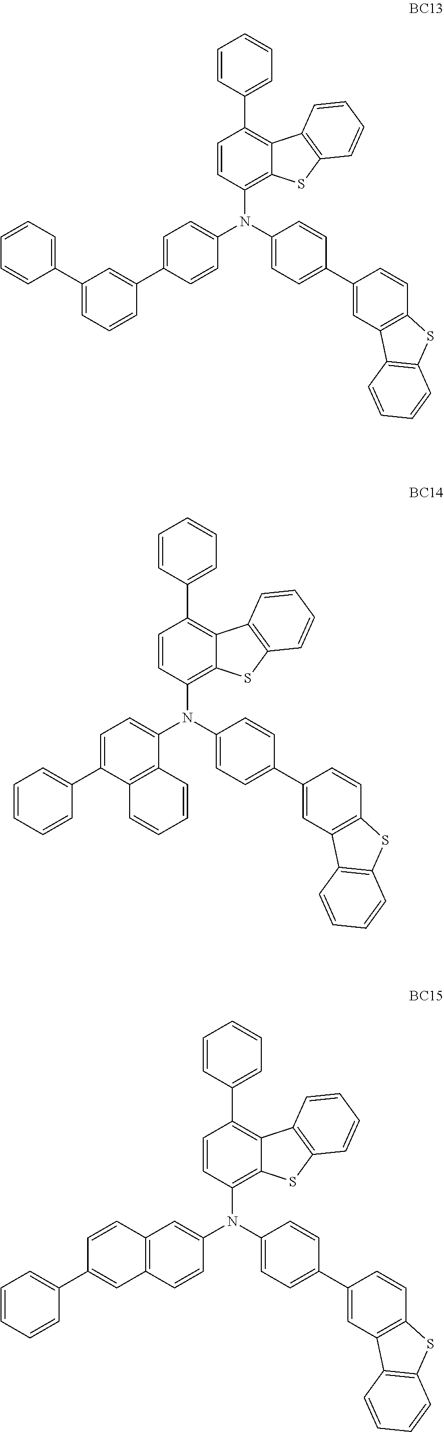 [SMILES: c1ccc(-c2cccc(-c3ccc(N(c4ccc(-c5ccc6sc7ccccc7c6c5)cc4)c4ccc(-c5ccccc5)c5c4sc4ccccc45)cc3)c2)cc1 ; c1ccc(-c2ccc(N(c3ccc(-c4ccc5sc6ccccc6c5c4)cc3)c3ccc(-c4ccccc4)c4c3sc3ccccc34)c3ccccc23)cc1 ; c1ccc(-c2ccc3cc(N(c4ccc(-c5ccc6sc7ccccc7c6c5)cc4)c4ccc(-c5ccccc5)c5c4sc4ccccc45)ccc3c2)cc1]

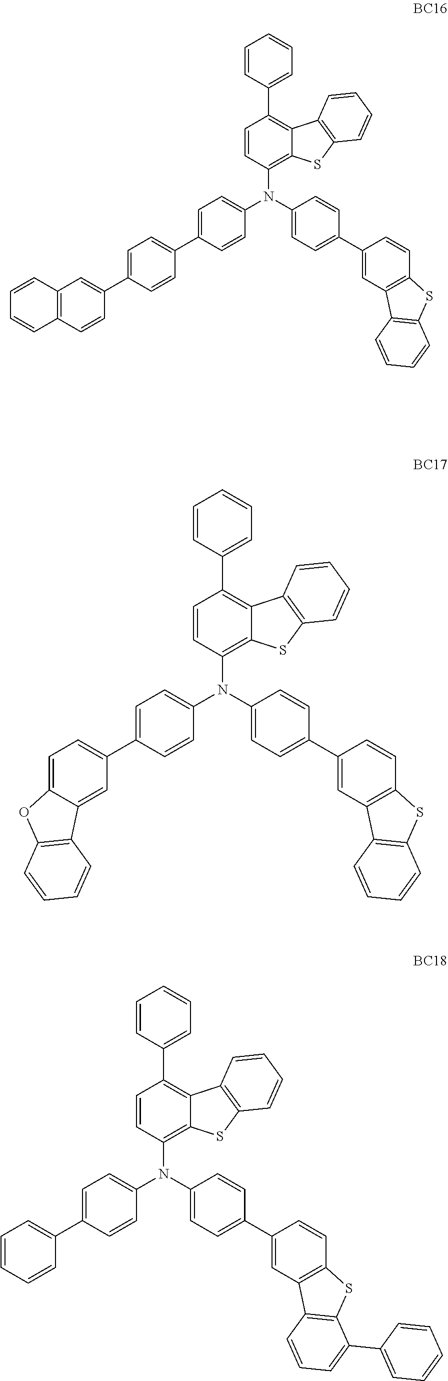 [SMILES: c1ccc(-c2ccc(N(c3ccc(-c4ccc(-c5ccc6ccccc6c5)cc4)cc3)c3ccc(-c4ccc5sc6ccccc6c5c4)cc3)c3sc4ccccc4c23)cc1 ; c1ccc(-c2ccc(N(c3ccc(-c4ccc5oc6ccccc6c5c4)cc3)c3ccc(-c4ccc5sc6ccccc6c5c4)cc3)c3sc4ccccc4c23)cc1 ; c1ccc(-c2ccc(N(c3ccc(-c4ccc5sc6c(-c7ccccc7)cccc6c5c4)cc3)c3ccc(-c4ccccc4)c4c3sc3ccccc34)cc2)cc1]

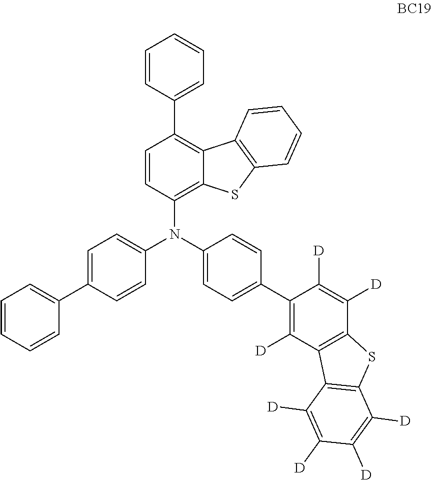 [SMILES: [2H]c1c([2H])c([2H])c2c(sc3c([2H])c([2H])c(-c4ccc(N(c5ccc(-c6ccccc6)cc5)c5ccc(-c6ccccc6)c6c5sc5ccccc56)cc4)c([2H])c32)c1[2H]]

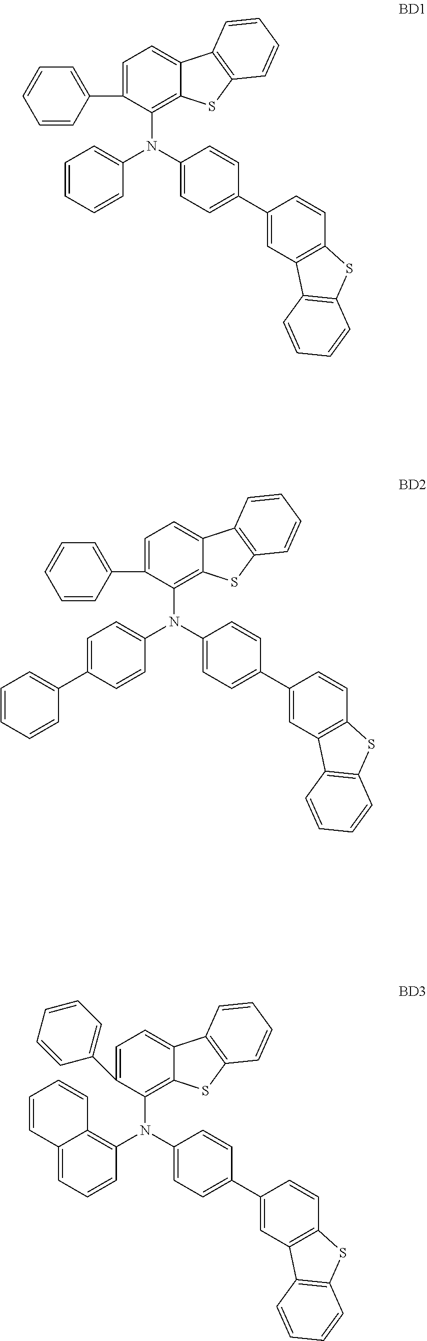 [SMILES: c1ccc(-c2ccc3c(sc4ccccc43)c2N(c2ccccc2)c2ccc(-c3ccc4sc5ccccc5c4c3)cc2)cc1 ; c1ccc(-c2ccc(N(c3ccc(-c4ccc5sc6ccccc6c5c4)cc3)c3c(-c4ccccc4)ccc4c3sc3ccccc34)cc2)cc1 ; c1ccc(-c2ccc3c(sc4ccccc43)c2N(c2ccc(-c3ccc4sc5ccccc5c4c3)cc2)c2cccc3ccccc23)cc1]

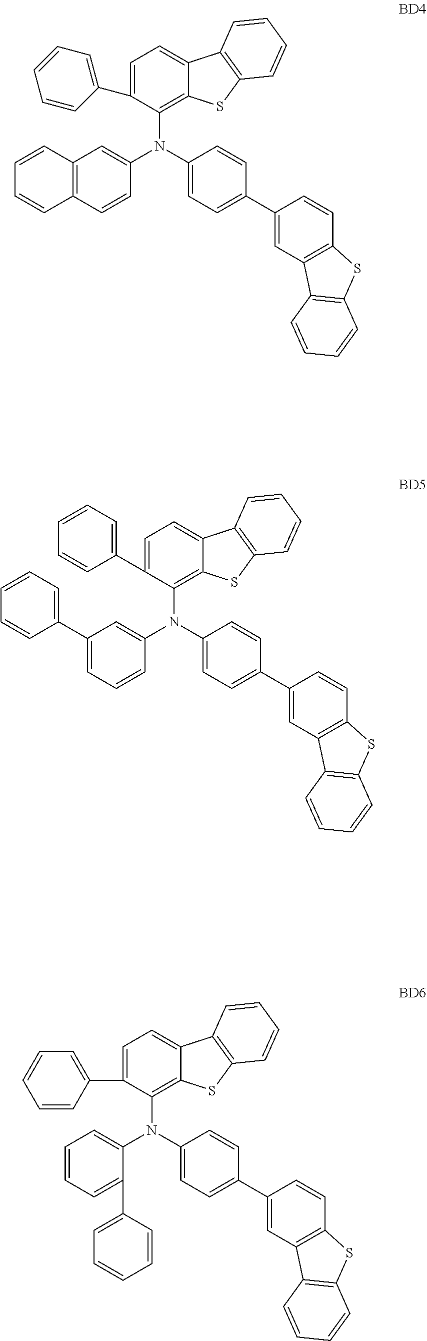 [SMILES: c1ccc(-c2ccc3c(sc4ccccc43)c2N(c2ccc(-c3ccc4sc5ccccc5c4c3)cc2)c2ccc3ccccc3c2)cc1 ; c1ccc(-c2cccc(N(c3ccc(-c4ccc5sc6ccccc6c5c4)cc3)c3c(-c4ccccc4)ccc4c3sc3ccccc34)c2)cc1 ; c1ccc(-c2ccccc2N(c2ccc(-c3ccc4sc5ccccc5c4c3)cc2)c2c(-c3ccccc3)ccc3c2sc2ccccc23)cc1]

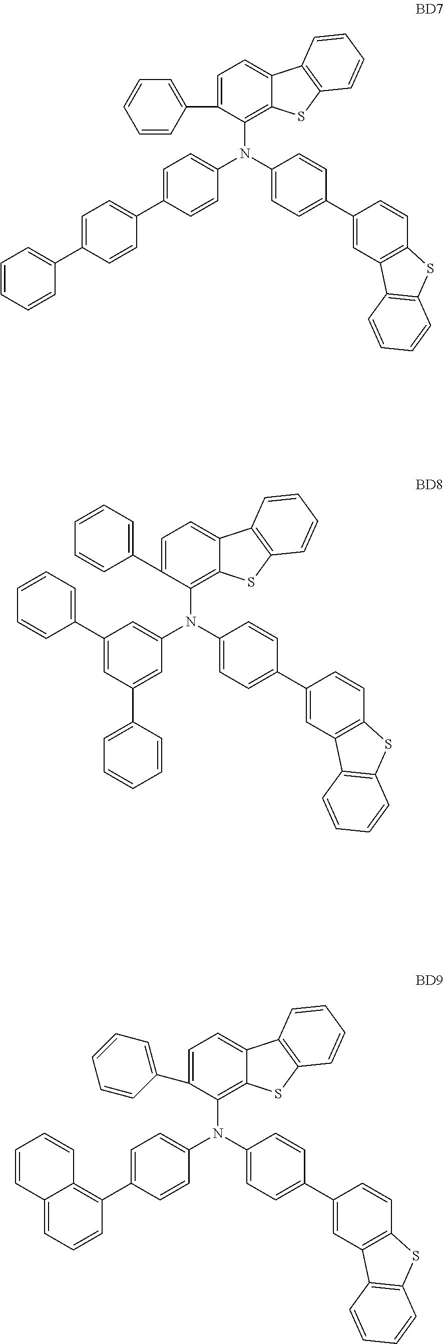 [SMILES: c1ccc(-c2ccc(-c3ccc(N(c4ccc(-c5ccc6sc7ccccc7c6c5)cc4)c4c(-c5ccccc5)ccc5c4sc4ccccc45)cc3)cc2)cc1 ; c1ccc(-c2cc(-c3ccccc3)cc(N(c3ccc(-c4ccc5sc6ccccc6c5c4)cc3)c3c(-c4ccccc4)ccc4c3sc3ccccc34)c2)cc1 ; c1ccc(-c2ccc3c(sc4ccccc43)c2N(c2ccc(-c3ccc4sc5ccccc5c4c3)cc2)c2ccc(-c3cccc4ccccc34)cc2)cc1]

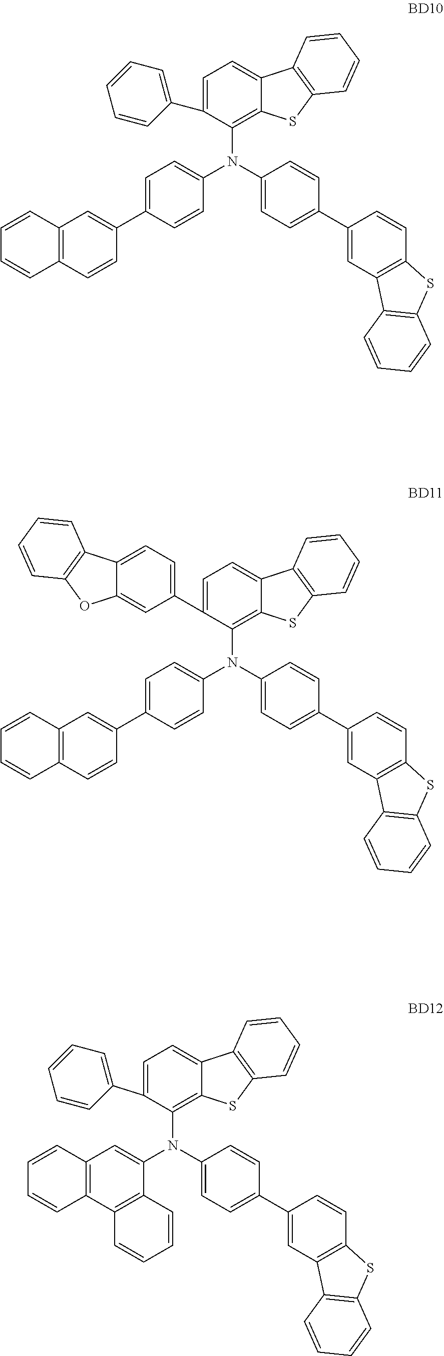 [SMILES: c1ccc(-c2ccc3c(sc4ccccc43)c2N(c2ccc(-c3ccc4ccccc4c3)cc2)c2ccc(-c3ccc4sc5ccccc5c4c3)cc2)cc1 ; c1ccc2cc(-c3ccc(N(c4ccc(-c5ccc6sc7ccccc7c6c5)cc4)c4c(-c5ccc6c(c5)oc5ccccc56)ccc5c4sc4ccccc45)cc3)ccc2c1 ; c1ccc(-c2ccc3c(sc4ccccc43)c2N(c2ccc(-c3ccc4sc5ccccc5c4c3)cc2)c2cc3ccccc3c3ccccc23)cc1]

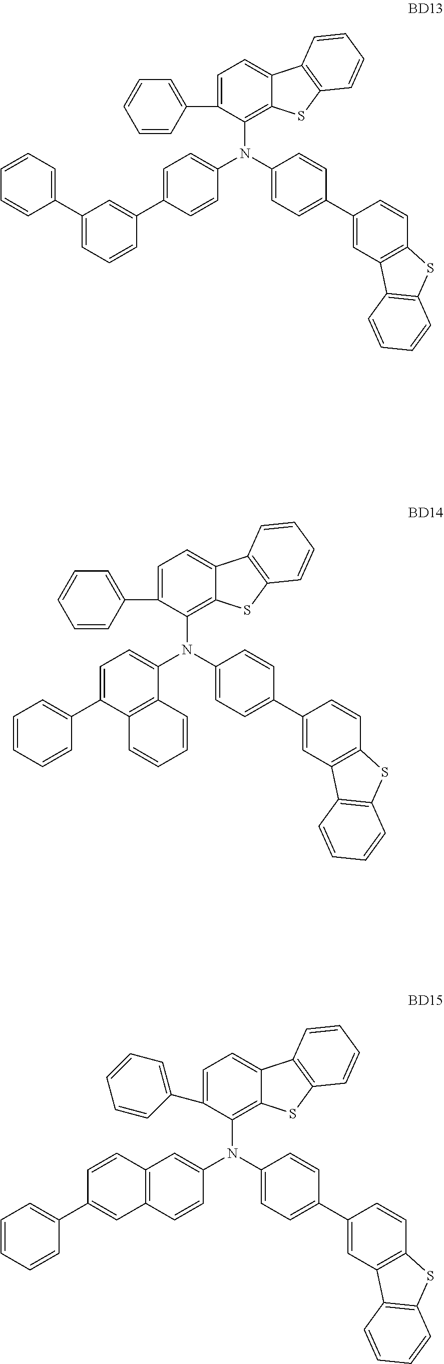 [SMILES: c1ccc(-c2cccc(-c3ccc(N(c4ccc(-c5ccc6sc7ccccc7c6c5)cc4)c4c(-c5ccccc5)ccc5c4sc4ccccc45)cc3)c2)cc1 ; c1ccc(-c2ccc3c(sc4ccccc43)c2N(c2ccc(-c3ccc4sc5ccccc5c4c3)cc2)c2ccc(-c3ccccc3)c3ccccc23)cc1 ; c1ccc(-c2ccc3cc(N(c4ccc(-c5ccc6sc7ccccc7c6c5)cc4)c4c(-c5ccccc5)ccc5c4sc4ccccc45)ccc3c2)cc1]

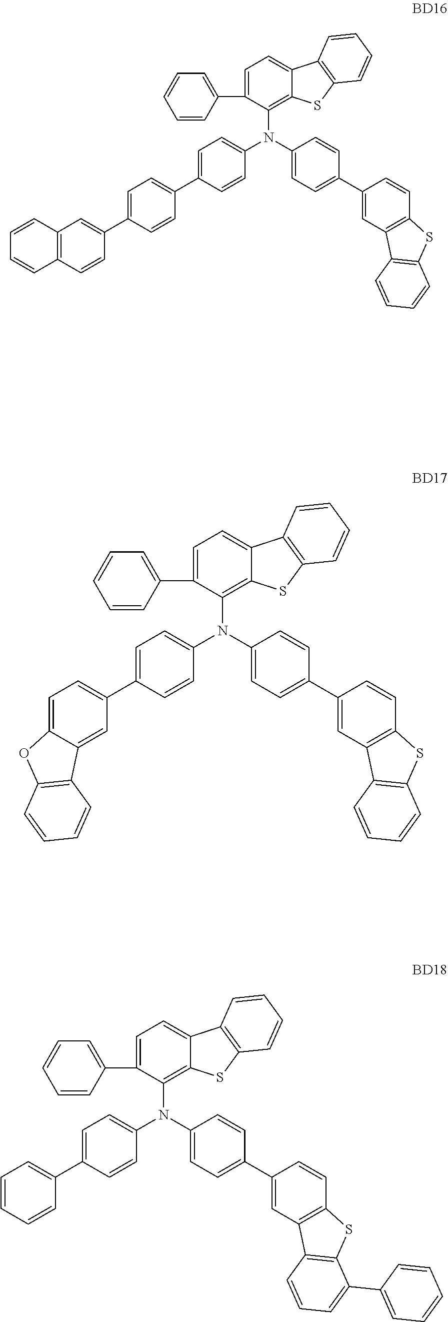 [SMILES: c1ccc(-c2ccc3c(sc4ccccc43)c2N(c2ccc(-c3ccc(-c4ccc5ccccc5c4)cc3)cc2)c2ccc(-c3ccc4sc5ccccc5c4c3)cc2)cc1 ; c1ccc(-c2ccc3c(sc4ccccc43)c2N(c2ccc(-c3ccc4oc5ccccc5c4c3)cc2)c2ccc(-c3ccc4sc5ccccc5c4c3)cc2)cc1 ; c1ccc(-c2ccc(N(c3ccc(-c4ccc5sc6c(-c7ccccc7)cccc6c5c4)cc3)c3c(-c4ccccc4)ccc4c3sc3ccccc34)cc2)cc1]

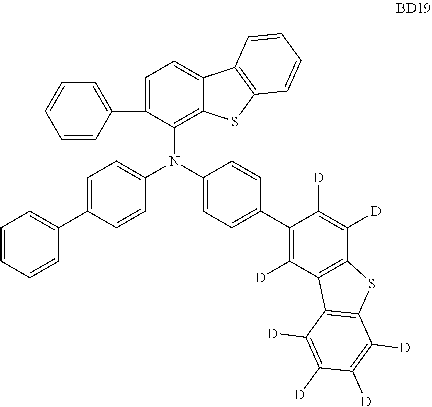 [SMILES: [2H]c1c([2H])c([2H])c2c(sc3c([2H])c([2H])c(-c4ccc(N(c5ccc(-c6ccccc6)cc5)c5c(-c6ccccc6)ccc6c5sc5ccccc56)cc4)c([2H])c32)c1[2H]]

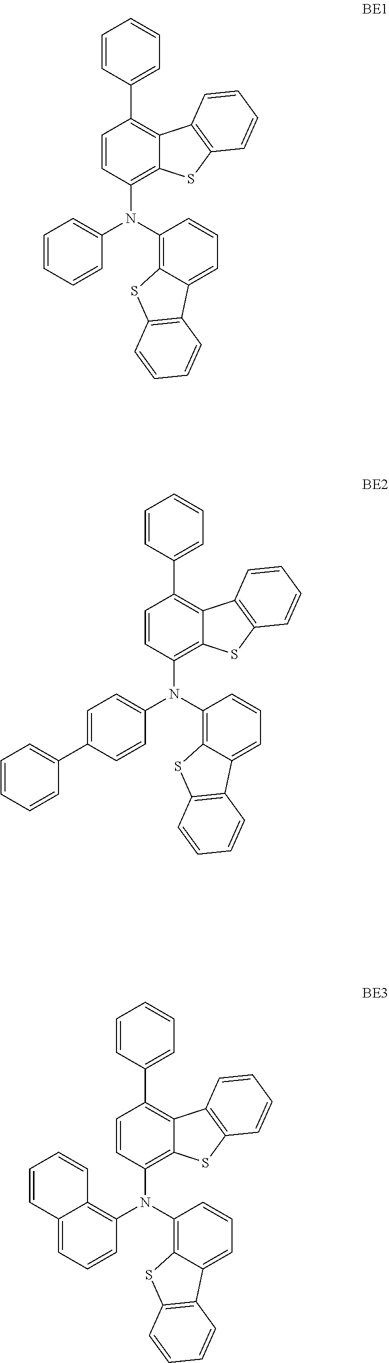 [SMILES: c1ccc(-c2ccc(N(c3ccccc3)c3cccc4c3sc3ccccc34)c3sc4ccccc4c23)cc1 ; c1ccc(-c2ccc(N(c3cccc4c3sc3ccccc34)c3ccc(-c4ccccc4)c4c3sc3ccccc34)cc2)cc1 ; c1ccc(-c2ccc(N(c3cccc4ccccc34)c3cccc4c3sc3ccccc34)c3sc4ccccc4c23)cc1]

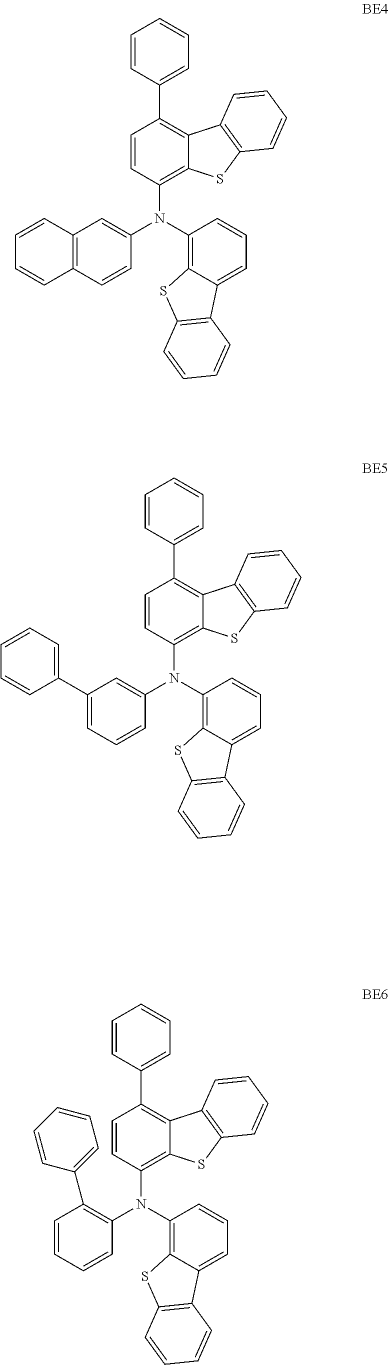 [SMILES: c1ccc(-c2ccc(N(c3ccc4ccccc4c3)c3cccc4c3sc3ccccc34)c3sc4ccccc4c23)cc1 ; c1ccc(-c2cccc(N(c3cccc4c3sc3ccccc34)c3ccc(-c4ccccc4)c4c3sc3ccccc34)c2)cc1 ; c1ccc(-c2ccccc2N(c2cccc3c2sc2ccccc23)c2ccc(-c3ccccc3)c3c2sc2ccccc23)cc1]

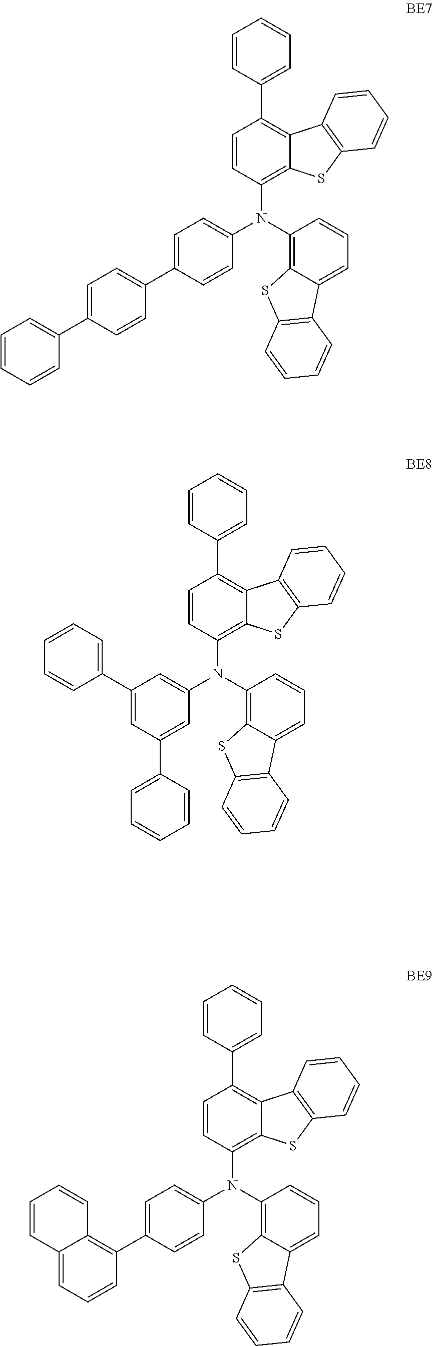 [SMILES: c1ccc(-c2ccc(-c3ccc(N(c4cccc5c4sc4ccccc45)c4ccc(-c5ccccc5)c5c4sc4ccccc45)cc3)cc2)cc1 ; c1ccc(-c2cc(-c3ccccc3)cc(N(c3cccc4c3sc3ccccc34)c3ccc(-c4ccccc4)c4c3sc3ccccc34)c2)cc1 ; c1ccc(-c2ccc(N(c3ccc(-c4cccc5ccccc45)cc3)c3cccc4c3sc3ccccc34)c3sc4ccccc4c23)cc1]

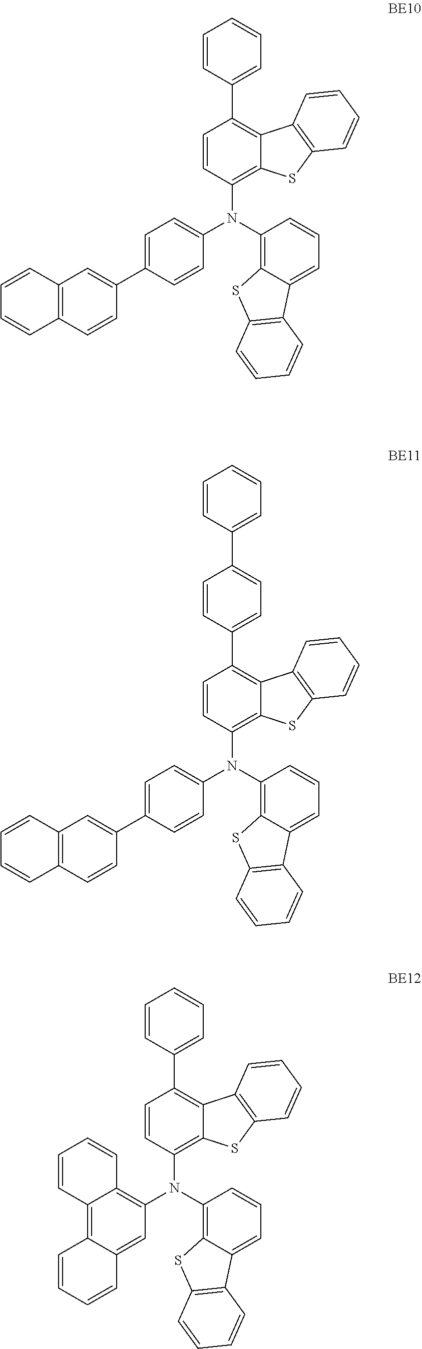 [SMILES: c1ccc(-c2ccc(N(c3ccc(-c4ccc5ccccc5c4)cc3)c3cccc4c3sc3ccccc34)c3sc4ccccc4c23)cc1 ; c1ccc(-c2ccc(-c3ccc(N(c4ccc(-c5ccc6ccccc6c5)cc4)c4cccc5c4sc4ccccc45)c4sc5ccccc5c34)cc2)cc1 ; c1ccc(-c2ccc(N(c3cc4ccccc4c4ccccc34)c3cccc4c3sc3ccccc34)c3sc4ccccc4c23)cc1]

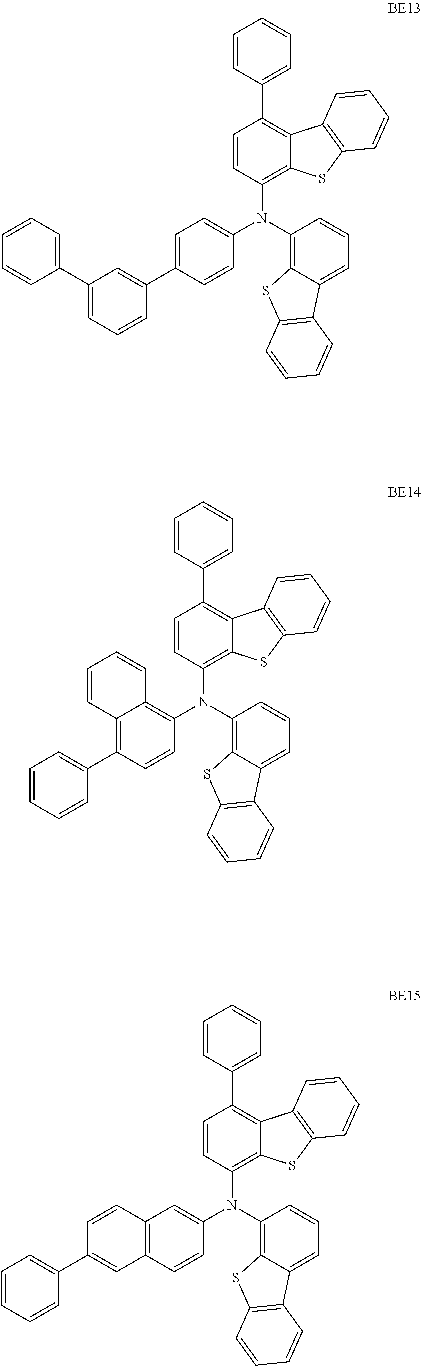 [SMILES: c1ccc(-c2cccc(-c3ccc(N(c4cccc5c4sc4ccccc45)c4ccc(-c5ccccc5)c5c4sc4ccccc45)cc3)c2)cc1 ; c1ccc(-c2ccc(N(c3cccc4c3sc3ccccc34)c3ccc(-c4ccccc4)c4c3sc3ccccc34)c3ccccc23)cc1 ; c1ccc(-c2ccc3cc(N(c4cccc5c4sc4ccccc45)c4ccc(-c5ccccc5)c5c4sc4ccccc45)ccc3c2)cc1]

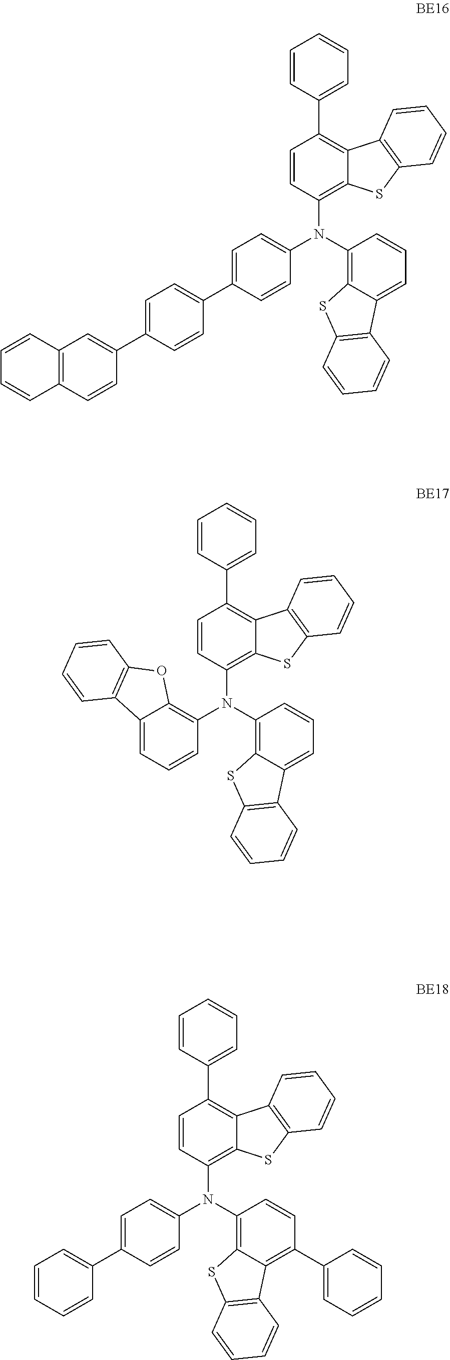 [SMILES: c1ccc(-c2ccc(N(c3ccc(-c4ccc(-c5ccc6ccccc6c5)cc4)cc3)c3cccc4c3sc3ccccc34)c3sc4ccccc4c23)cc1 ; c1ccc(-c2ccc(N(c3cccc4c3oc3ccccc34)c3cccc4c3sc3ccccc34)c3sc4ccccc4c23)cc1 ; c1ccc(-c2ccc(N(c3ccc(-c4ccccc4)c4c3sc3ccccc34)c3ccc(-c4ccccc4)c4c3sc3ccccc34)cc2)cc1]

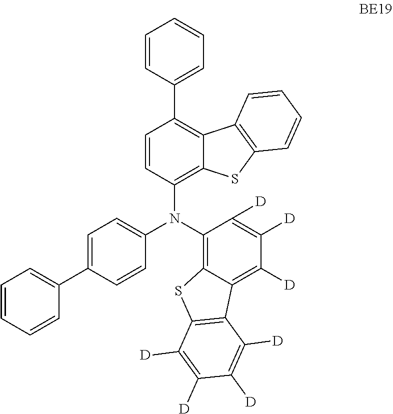 [SMILES: [2H]c1c([2H])c([2H])c2c(sc3c(N(c4ccc(-c5ccccc5)cc4)c4ccc(-c5ccccc5)c5c4sc4ccccc45)c([2H])c([2H])c([2H])c32)c1[2H]]

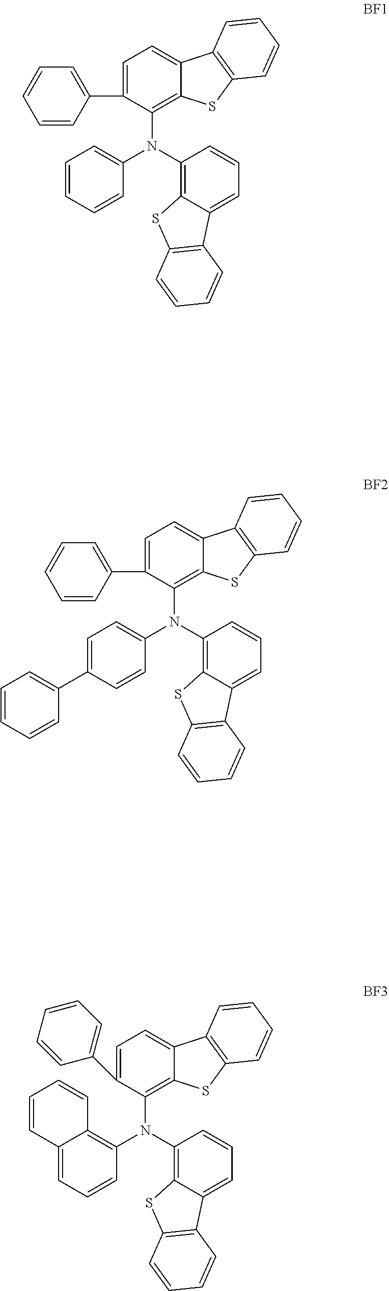 [SMILES: c1ccc(-c2ccc3c(sc4ccccc43)c2N(c2ccccc2)c2cccc3c2sc2ccccc23)cc1 ; c1ccc(-c2ccc(N(c3cccc4c3sc3ccccc34)c3c(-c4ccccc4)ccc4c3sc3ccccc34)cc2)cc1 ; c1ccc(-c2ccc3c(sc4ccccc43)c2N(c2cccc3ccccc23)c2cccc3c2sc2ccccc23)cc1]

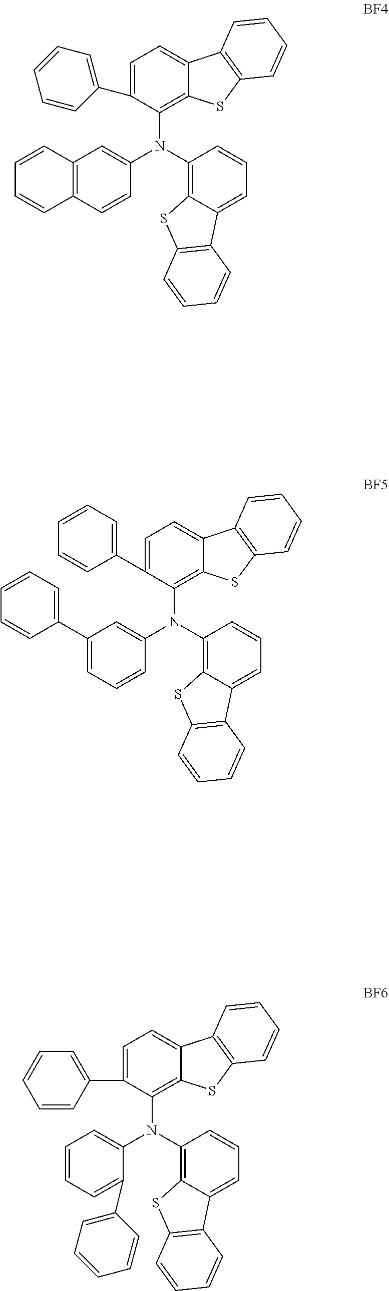 [SMILES: c1ccc(-c2ccc3c(sc4ccccc43)c2N(c2ccc3ccccc3c2)c2cccc3c2sc2ccccc23)cc1 ; c1ccc(-c2cccc(N(c3cccc4c3sc3ccccc34)c3c(-c4ccccc4)ccc4c3sc3ccccc34)c2)cc1 ; c1ccc(-c2ccccc2N(c2cccc3c2sc2ccccc23)c2c(-c3ccccc3)ccc3c2sc2ccccc23)cc1]

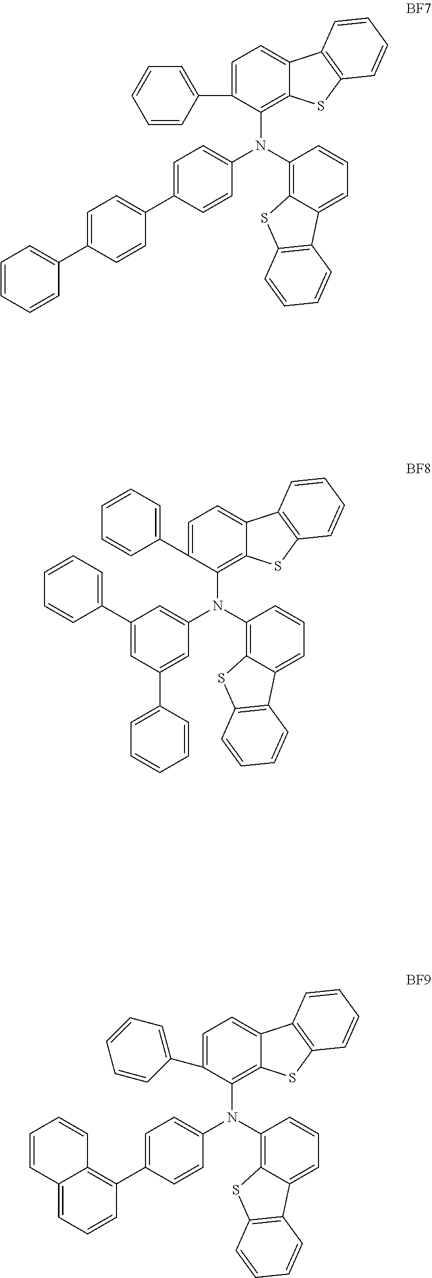 [SMILES: c1ccc(-c2ccc(-c3ccc(N(c4cccc5c4sc4ccccc45)c4c(-c5ccccc5)ccc5c4sc4ccccc45)cc3)cc2)cc1 ; c1ccc(-c2cc(-c3ccccc3)cc(N(c3cccc4c3sc3ccccc34)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2cccc3c2sc2ccccc23)cc1]

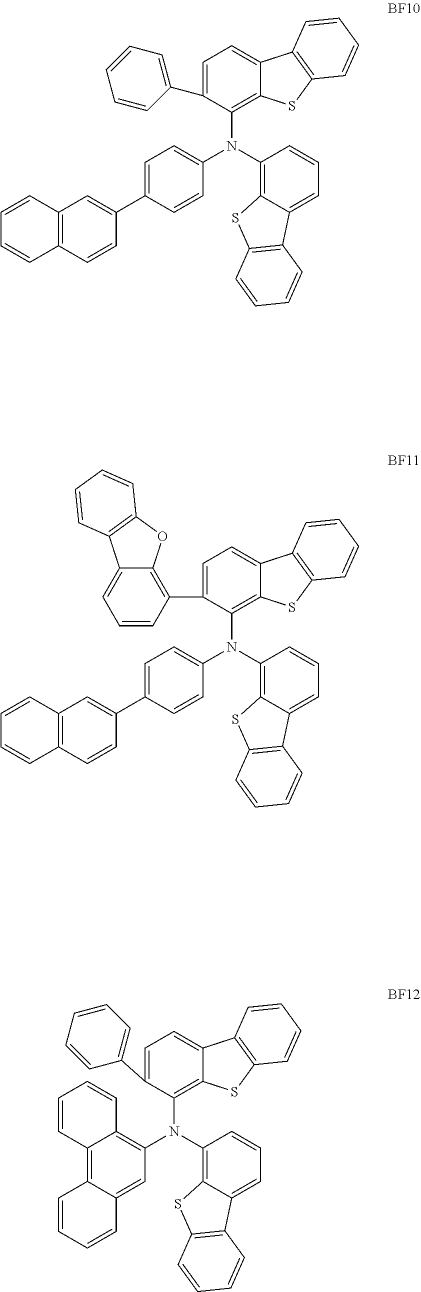 [SMILES: c1ccc(-c2ccc3c(sc4ccccc43)c2N(c2ccc(-c3ccc4ccccc4c3)cc2)c2cccc3c2sc2ccccc23)cc1 ; c1ccc2cc(-c3ccc(N(c4cccc5c4sc4ccccc45)c4c(-c5cccc6c5oc5ccccc56)ccc5c4sc4ccccc45)cc3)ccc2c1 ; c1ccc(-c2ccc3c(sc4ccccc43)c2N(c2cc3ccccc3c3ccccc23)c2cccc3c2sc2ccccc23)cc1]

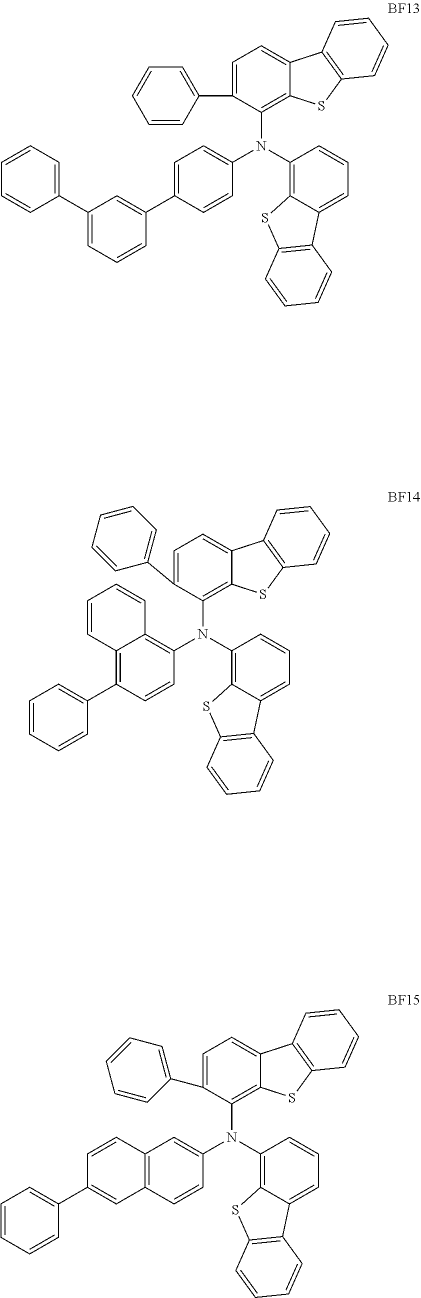 [SMILES: c1ccc(-c2cccc(-c3ccc(N(c4cccc5c4sc4ccccc45)c4c(-c5ccccc5)ccc5c4sc4ccccc45)cc3)c2)cc1 ; c1ccc(-c2ccc3c(sc4ccccc43)c2N(c2ccc(-c3ccccc3)c3ccccc23)c2cccc3c2sc2ccccc23)cc1 ; c1ccc(-c2ccc3cc(N(c4cccc5c4sc4ccccc45)c4c(-c5ccccc5)ccc5c4sc4ccccc45)ccc3c2)cc1]

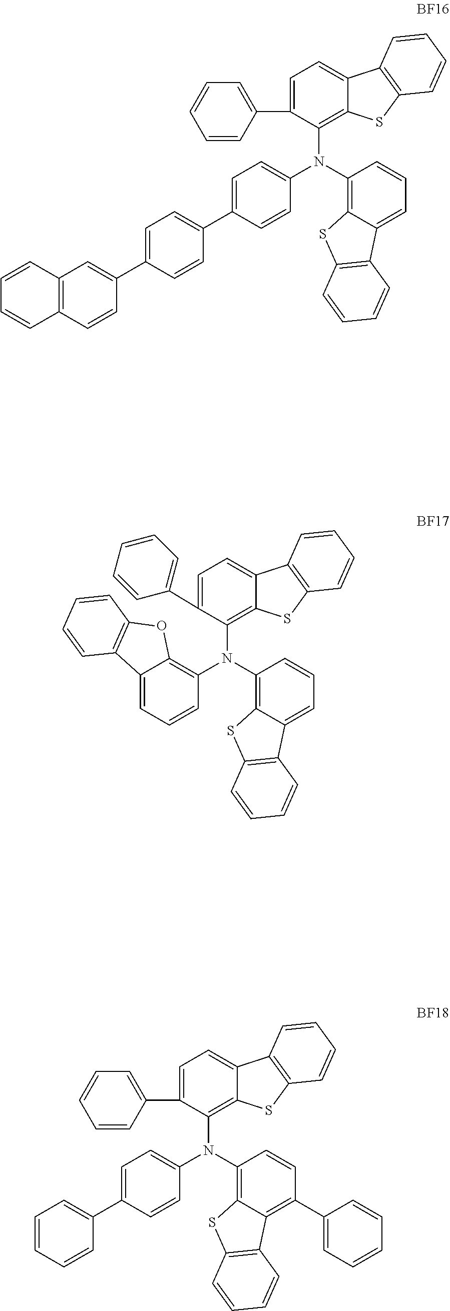 [SMILES: c1ccc(-c2ccc3c(sc4ccccc43)c2N(c2ccc(-c3ccc(-c4ccc5ccccc5c4)cc3)cc2)c2cccc3c2sc2ccccc23)cc1 ; c1ccc(-c2ccc3c(sc4ccccc43)c2N(c2cccc3c2oc2ccccc23)c2cccc3c2sc2ccccc23)cc1 ; c1ccc(-c2ccc(N(c3c(-c4ccccc4)ccc4c3sc3ccccc34)c3ccc(-c4ccccc4)c4c3sc3ccccc34)cc2)cc1]

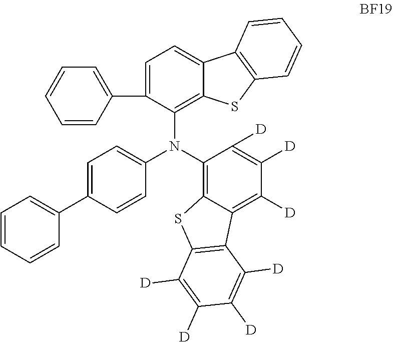 [SMILES: [2H]c1c([2H])c([2H])c2c(sc3c(N(c4ccc(-c5ccccc5)cc4)c4c(-c5ccccc5)ccc5c4sc4ccccc45)c([2H])c([2H])c([2H])c32)c1[2H]]

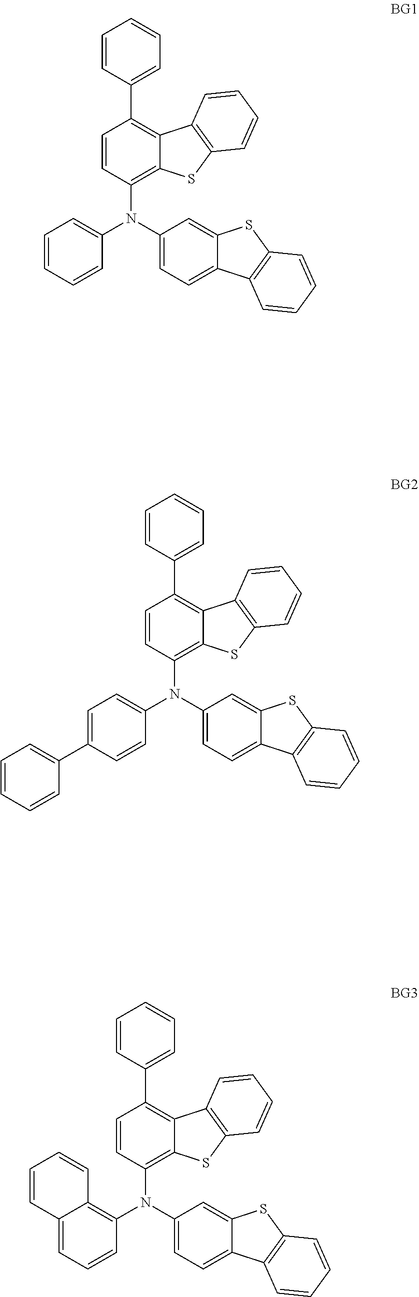 [SMILES: c1ccc(-c2ccc(N(c3ccccc3)c3ccc4c(c3)sc3ccccc34)c3sc4ccccc4c23)cc1 ; c1ccc(-c2ccc(N(c3ccc4c(c3)sc3ccccc34)c3ccc(-c4ccccc4)c4c3sc3ccccc34)cc2)cc1 ; c1ccc(-c2ccc(N(c3ccc4c(c3)sc3ccccc34)c3cccc4ccccc34)c3sc4ccccc4c23)cc1]

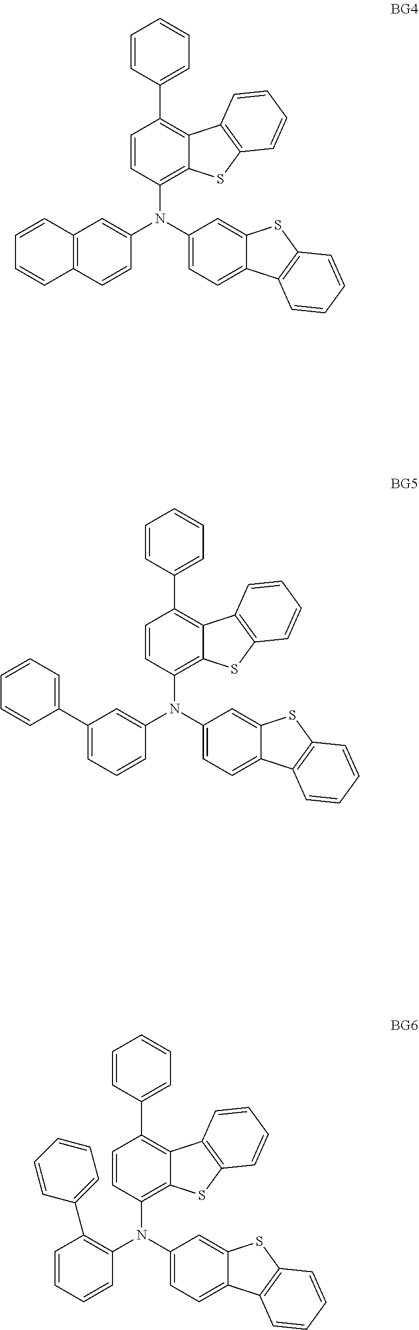 [SMILES: c1ccc(-c2ccc(N(c3ccc4ccccc4c3)c3ccc4c(c3)sc3ccccc34)c3sc4ccccc4c23)cc1 ; c1ccc(-c2cccc(N(c3ccc4c(c3)sc3ccccc34)c3ccc(-c4ccccc4)c4c3sc3ccccc34)c2)cc1 ; c1ccc(-c2ccccc2N(c2ccc3c(c2)sc2ccccc23)c2ccc(-c3ccccc3)c3c2sc2ccccc23)cc1]

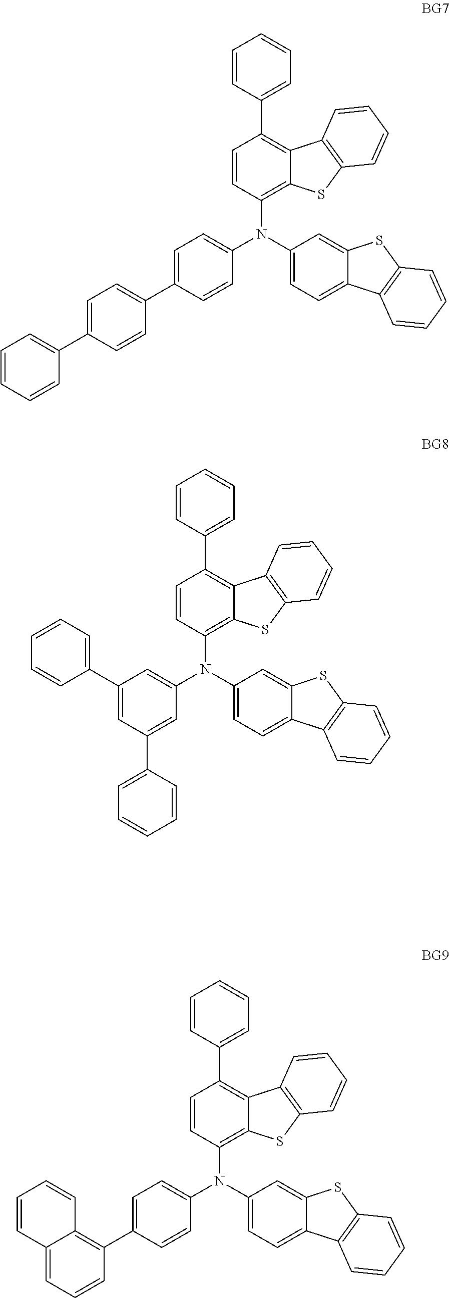 [SMILES: c1ccc(-c2ccc(-c3ccc(N(c4ccc5c(c4)sc4ccccc45)c4ccc(-c5ccccc5)c5c4sc4ccccc45)cc3)cc2)cc1 ; c1ccc(-c2cc(-c3ccccc3)cc(N(c3ccc4c(c3)sc3ccccc34)c3ccc(-c4ccccc4)c4c3sc3ccccc34)c2)cc1 ; c1ccc(-c2ccc(N(c3ccc(-c4cccc5ccccc45)cc3)c3ccc4c(c3)sc3ccccc34)c3sc4ccccc4c23)cc1]

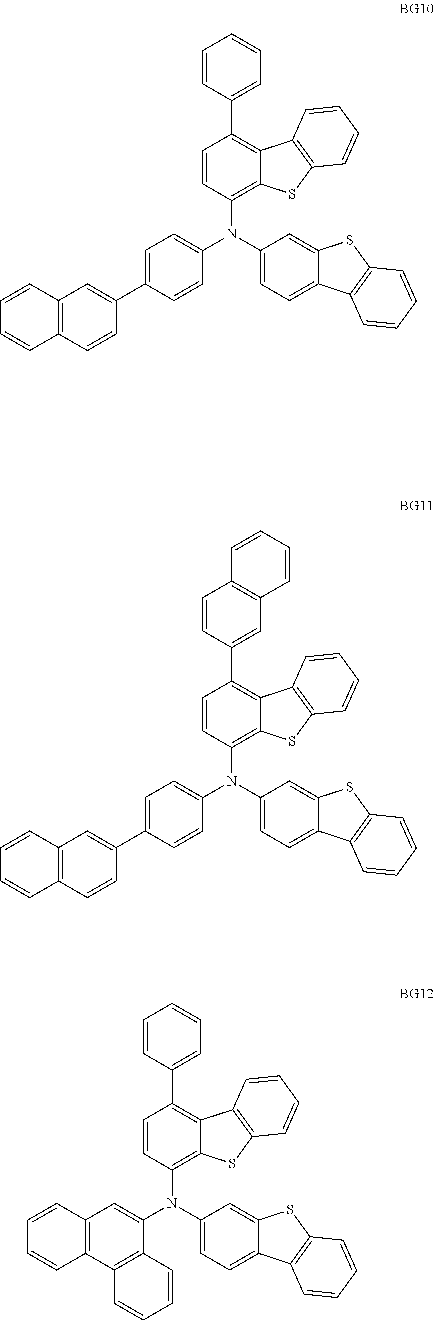 [SMILES: c1ccc(-c2ccc(N(c3ccc(-c4ccc5ccccc5c4)cc3)c3ccc4c(c3)sc3ccccc34)c3sc4ccccc4c23)cc1 ; c1ccc2cc(-c3ccc(N(c4ccc5c(c4)sc4ccccc45)c4ccc(-c5ccc6ccccc6c5)c5c4sc4ccccc45)cc3)ccc2c1 ; c1ccc(-c2ccc(N(c3ccc4c(c3)sc3ccccc34)c3cc4ccccc4c4ccccc34)c3sc4ccccc4c23)cc1]

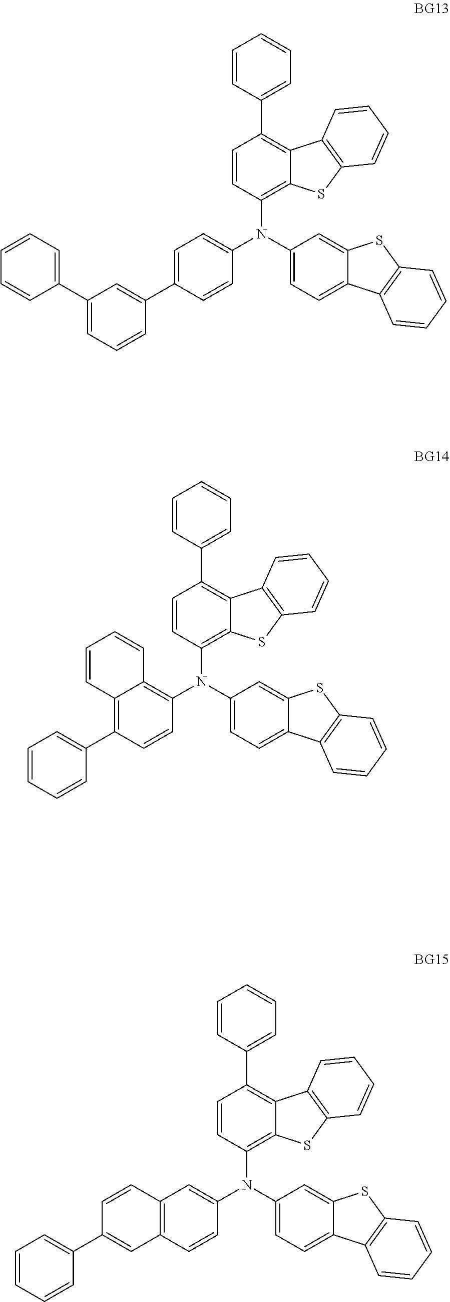 [SMILES: c1ccc(-c2cccc(-c3ccc(N(c4ccc5c(c4)sc4ccccc45)c4ccc(-c5ccccc5)c5c4sc4ccccc45)cc3)c2)cc1 ; c1ccc(-c2ccc(N(c3ccc4c(c3)sc3ccccc34)c3ccc(-c4ccccc4)c4c3sc3ccccc34)c3ccccc23)cc1 ; c1ccc(-c2ccc3cc(N(c4ccc5c(c4)sc4ccccc45)c4ccc(-c5ccccc5)c5c4sc4ccccc45)ccc3c2)cc1]

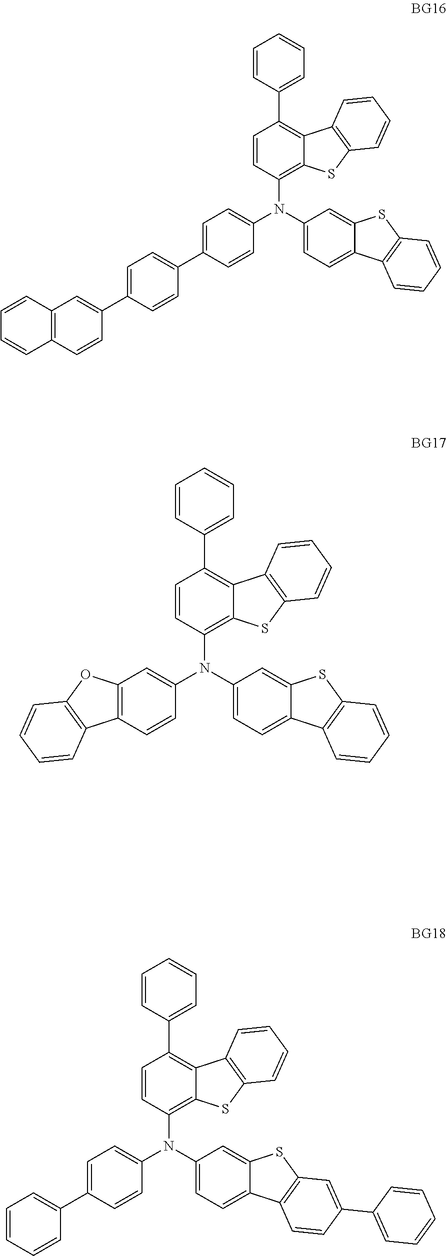 [SMILES: c1ccc(-c2ccc(N(c3ccc(-c4ccc(-c5ccc6ccccc6c5)cc4)cc3)c3ccc4c(c3)sc3ccccc34)c3sc4ccccc4c23)cc1 ; c1ccc(-c2ccc(N(c3ccc4c(c3)oc3ccccc34)c3ccc4c(c3)sc3ccccc34)c3sc4ccccc4c23)cc1 ; c1ccc(-c2ccc(N(c3ccc4c(c3)sc3cc(-c5ccccc5)ccc34)c3ccc(-c4ccccc4)c4c3sc3ccccc34)cc2)cc1]

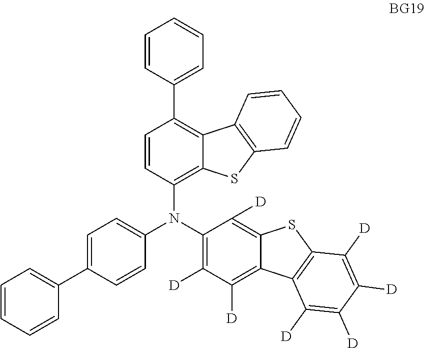 [SMILES: [2H]c1c([2H])c([2H])c2c(sc3c([2H])c(N(c4ccc(-c5ccccc5)cc4)c4ccc(-c5ccccc5)c5c4sc4ccccc45)c([2H])c([2H])c32)c1[2H]]

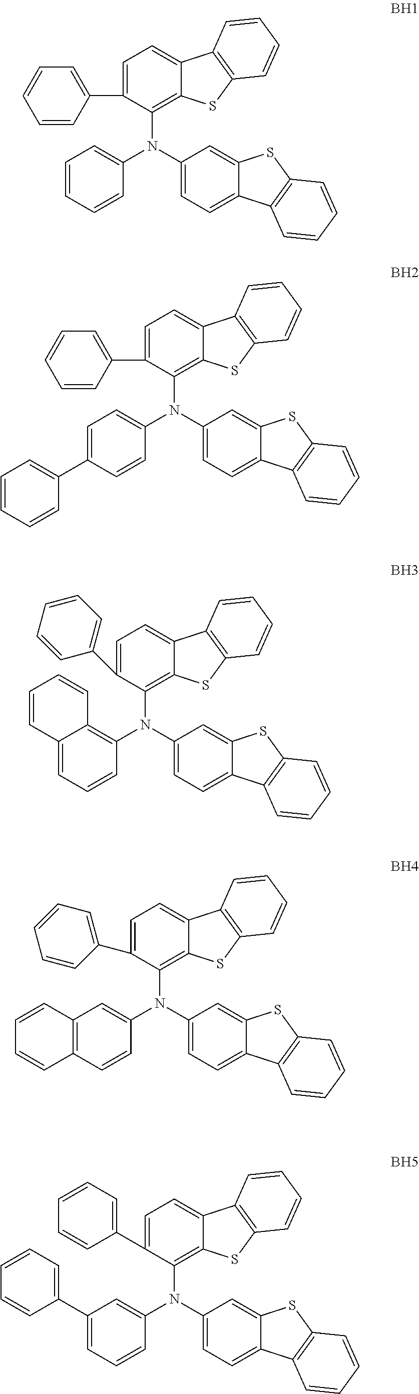

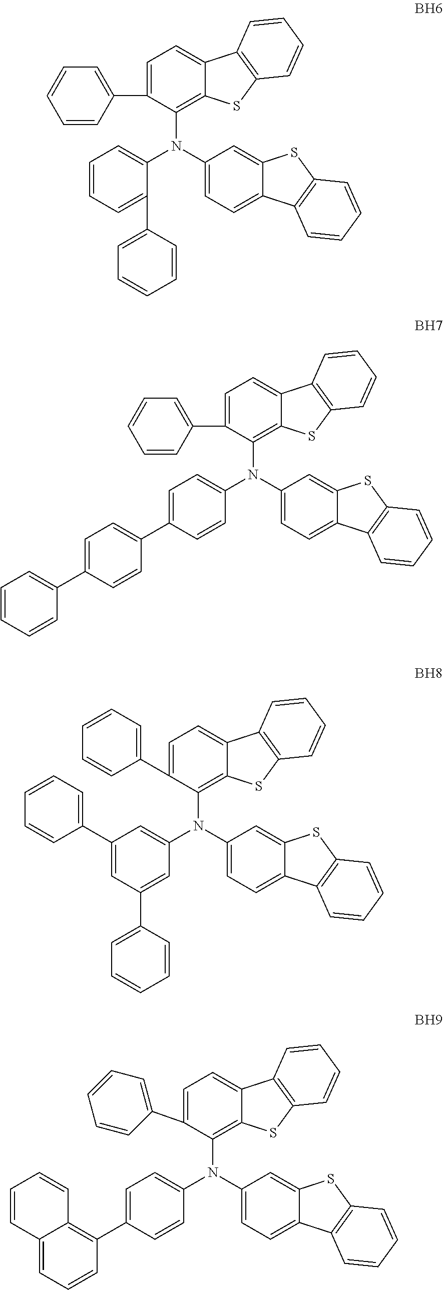 [SMILES: c1ccc(-c2ccccc2N(c2ccc3c(c2)sc2ccccc23)c2c(-c3ccccc3)ccc3c2sc2ccccc23)cc1 ; c1ccc(-c2ccc(-c3ccc(N(c4ccc5c(c4)sc4ccccc45)c4c(-c5ccccc5)ccc5c4sc4ccccc45)cc3)cc2)cc1 ; c1ccc(-c2cc(-c3ccccc3)cc(N(c3ccc4c(c3)sc3ccccc34)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2ccc3c(c2)sc2ccccc23)cc1]

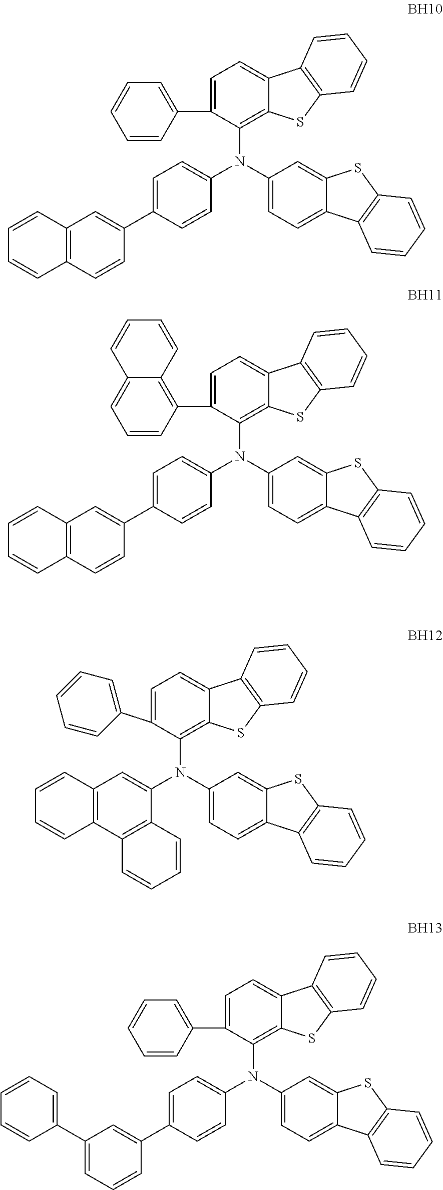 [SMILES: c1ccc(-c2ccc3c(sc4ccccc43)c2N(c2ccc(-c3ccc4ccccc4c3)cc2)c2ccc3c(c2)sc2ccccc23)cc1 ; c1ccc2cc(-c3ccc(N(c4ccc5c(c4)sc4ccccc45)c4c(-c5cccc6ccccc56)ccc5c4sc4ccccc45)cc3)ccc2c1 ; c1ccc(-c2ccc3c(sc4ccccc43)c2N(c2ccc3c(c2)sc2ccccc23)c2cc3ccccc3c3ccccc23)cc1 ; c1ccc(-c2cccc(-c3ccc(N(c4ccc5c(c4)sc4ccccc45)c4c(-c5ccccc5)ccc5c4sc4ccccc45)cc3)c2)cc1]

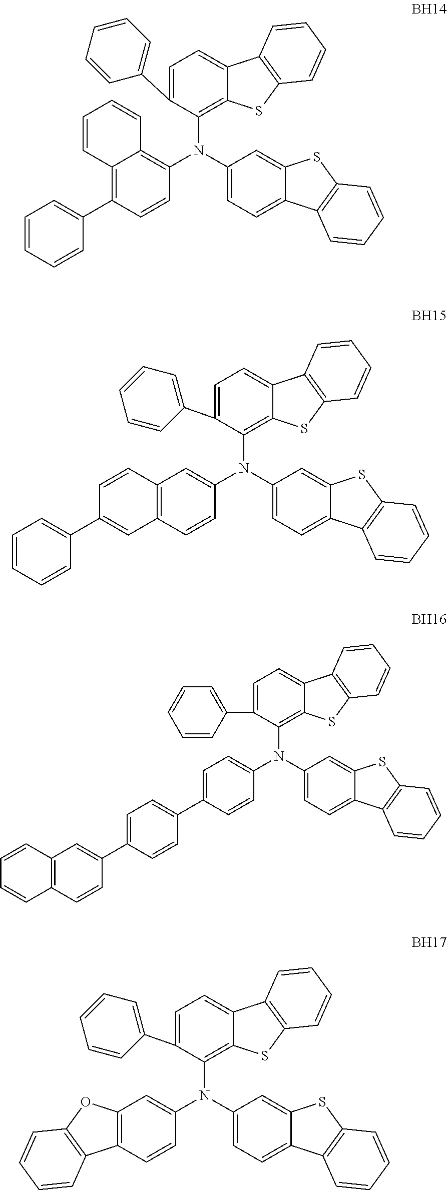 [SMILES: c1ccc(-c2ccc3c(sc4ccccc43)c2N(c2ccc3c(c2)sc2ccccc23)c2ccc(-c3ccccc3)c3ccccc23)cc1 ; c1ccc(-c2ccc3cc(N(c4ccc5c(c4)sc4ccccc45)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc3c(c2)sc2ccccc23)cc1 ; c1ccc(-c2ccc3c(sc4ccccc43)c2N(c2ccc3c(c2)oc2ccccc23)c2ccc3c(c2)sc2ccccc23)cc1]

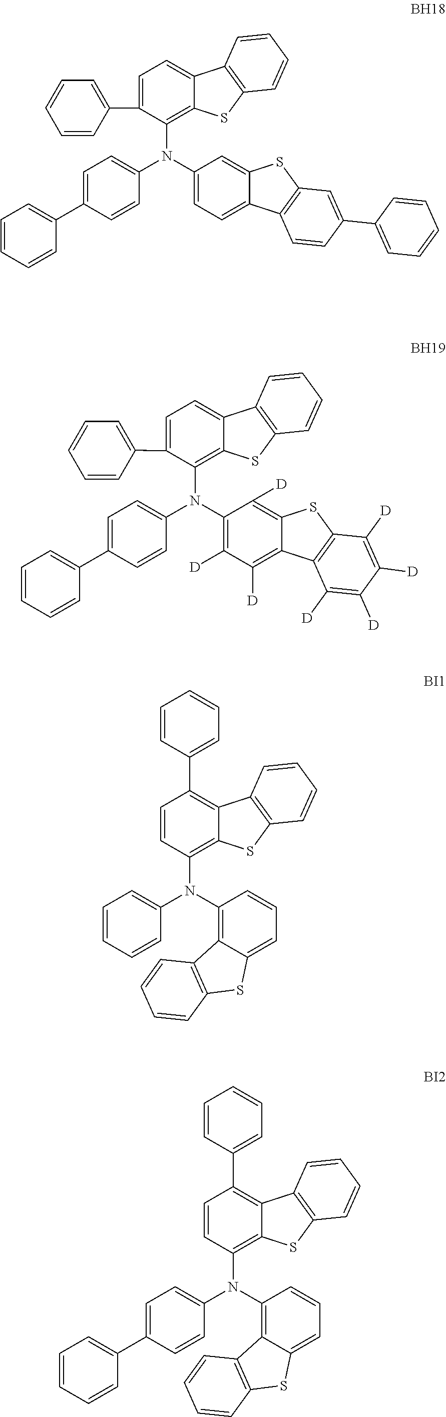 [SMILES: c1ccc(-c2ccc(N(c3ccc4c(c3)sc3cc(-c5ccccc5)ccc34)c3c(-c4ccccc4)ccc4c3sc3ccccc34)cc2)cc1 ; [2H]c1c([2H])c([2H])c2c(sc3c([2H])c(N(c4ccc(-c5ccccc5)cc4)c4c(-c5ccccc5)ccc5c4sc4ccccc45)c([2H])c([2H])c32)c1[2H] ; c1ccc(-c2ccc(N(c3ccccc3)c3cccc4sc5ccccc5c34)c3sc4ccccc4c23)cc1 ; c1ccc(-c2ccc(N(c3ccc(-c4ccccc4)c4c3sc3ccccc34)c3cccc4sc5ccccc5c34)cc2)cc1]

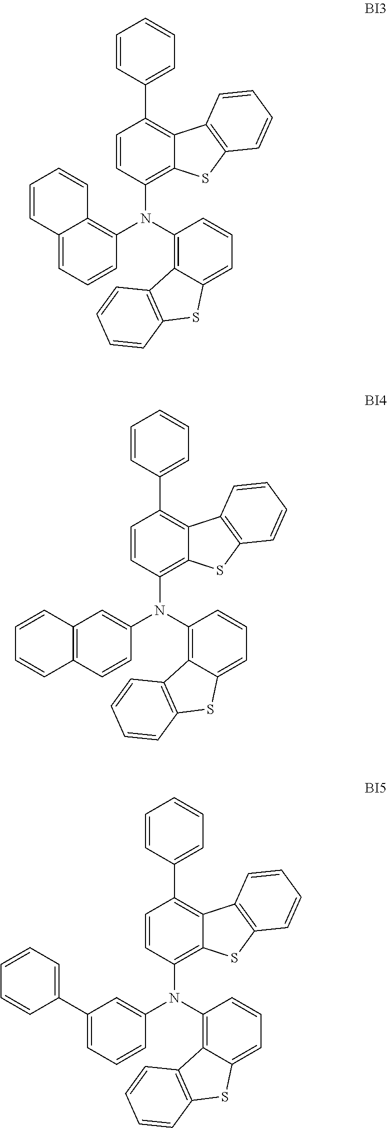 [SMILES: c1ccc(-c2ccc(N(c3cccc4ccccc34)c3cccc4sc5ccccc5c34)c3sc4ccccc4c23)cc1 ; c1ccc(-c2ccc(N(c3ccc4ccccc4c3)c3cccc4sc5ccccc5c34)c3sc4ccccc4c23)cc1 ; c1ccc(-c2cccc(N(c3ccc(-c4ccccc4)c4c3sc3ccccc34)c3cccc4sc5ccccc5c34)c2)cc1]

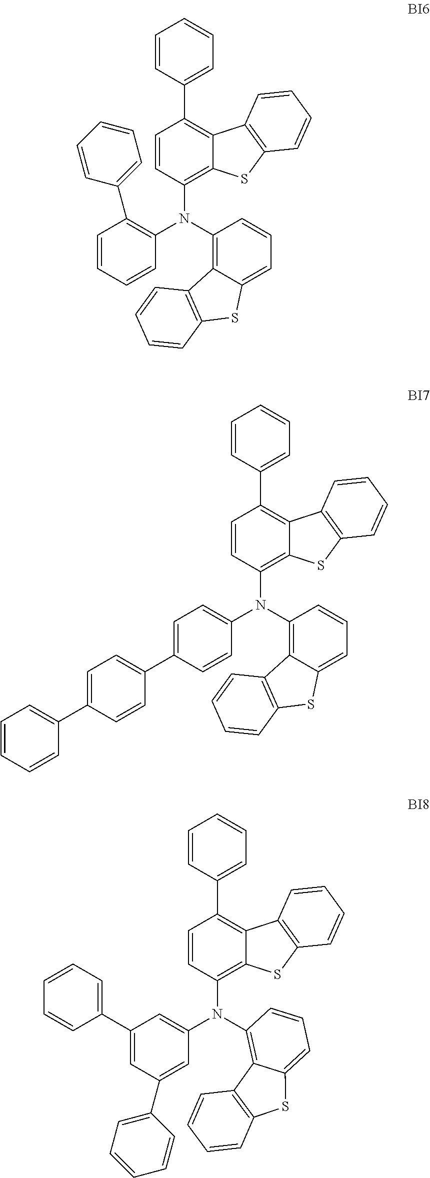 [SMILES: c1ccc(-c2ccccc2N(c2ccc(-c3ccccc3)c3c2sc2ccccc23)c2cccc3sc4ccccc4c23)cc1 ; c1ccc(-c2ccc(-c3ccc(N(c4ccc(-c5ccccc5)c5c4sc4ccccc45)c4cccc5sc6ccccc6c45)cc3)cc2)cc1 ; c1ccc(-c2cc(-c3ccccc3)cc(N(c3ccc(-c4ccccc4)c4c3sc3ccccc34)c3cccc4sc5ccccc5c34)c2)cc1]

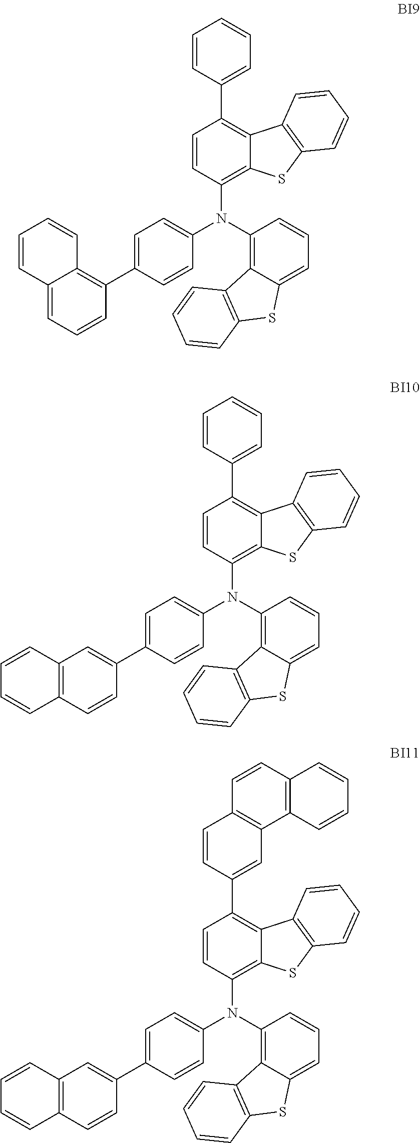 [SMILES: c1ccc(-c2ccc(N(c3ccc(-c4cccc5ccccc45)cc3)c3cccc4sc5ccccc5c34)c3sc4ccccc4c23)cc1 ; c1ccc(-c2ccc(N(c3ccc(-c4ccc5ccccc5c4)cc3)c3cccc4sc5ccccc5c34)c3sc4ccccc4c23)cc1 ; c1ccc2cc(-c3ccc(N(c4ccc(-c5ccc6ccc7ccccc7c6c5)c5c4sc4ccccc45)c4cccc5sc6ccccc6c45)cc3)ccc2c1]

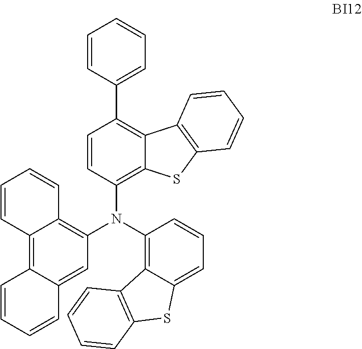 [SMILES: c1ccc(-c2ccc(N(c3cc4ccccc4c4ccccc34)c3cccc4sc5ccccc5c34)c3sc4ccccc4c23)cc1]

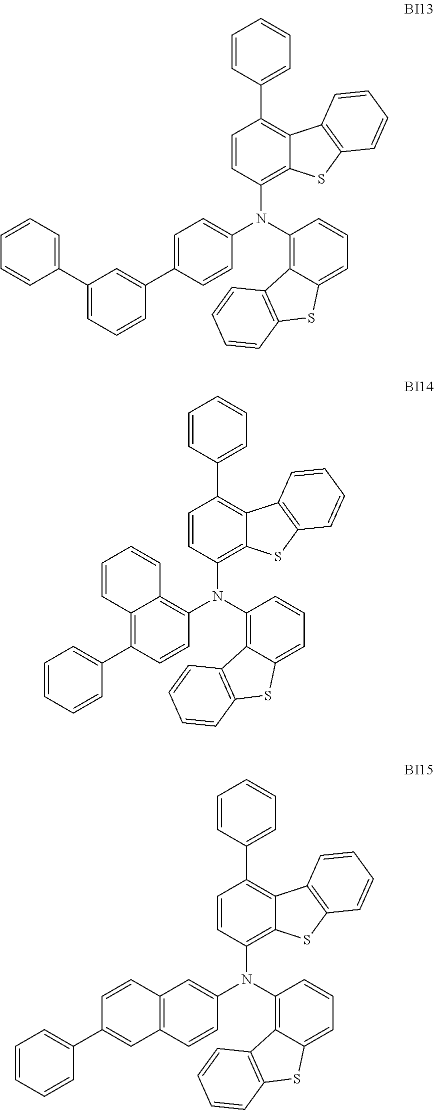 [SMILES: c1ccc(-c2cccc(-c3ccc(N(c4ccc(-c5ccccc5)c5c4sc4ccccc45)c4cccc5sc6ccccc6c45)cc3)c2)cc1 ; c1ccc(-c2ccc(N(c3ccc(-c4ccccc4)c4c3sc3ccccc34)c3cccc4sc5ccccc5c34)c3ccccc23)cc1 ; c1ccc(-c2ccc3cc(N(c4ccc(-c5ccccc5)c5c4sc4ccccc45)c4cccc5sc6ccccc6c45)ccc3c2)cc1]

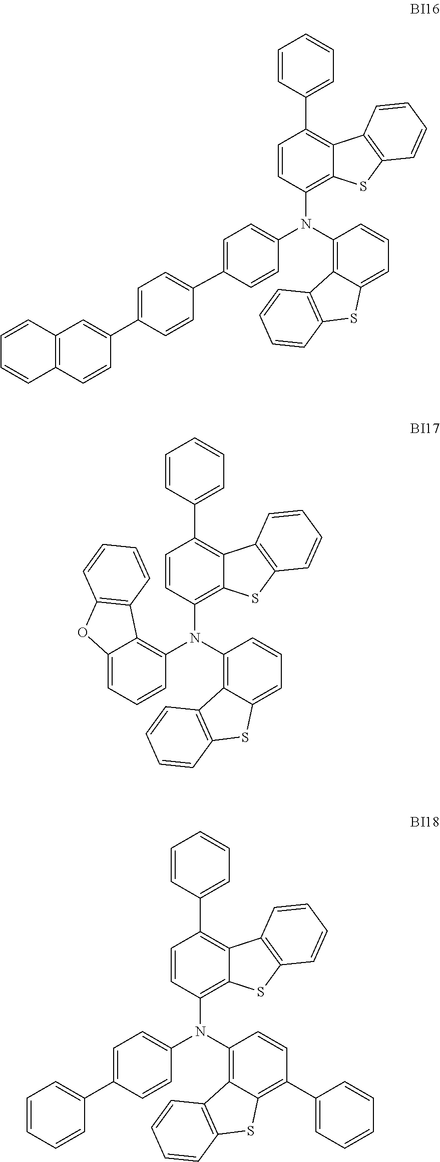 [SMILES: c1ccc(-c2ccc(N(c3ccc(-c4ccc(-c5ccc6ccccc6c5)cc4)cc3)c3cccc4sc5ccccc5c34)c3sc4ccccc4c23)cc1 ; c1ccc(-c2ccc(N(c3cccc4oc5ccccc5c34)c3cccc4sc5ccccc5c34)c3sc4ccccc4c23)cc1 ; c1ccc(-c2ccc(N(c3ccc(-c4ccccc4)c4c3sc3ccccc34)c3ccc(-c4ccccc4)c4sc5ccccc5c34)cc2)cc1]

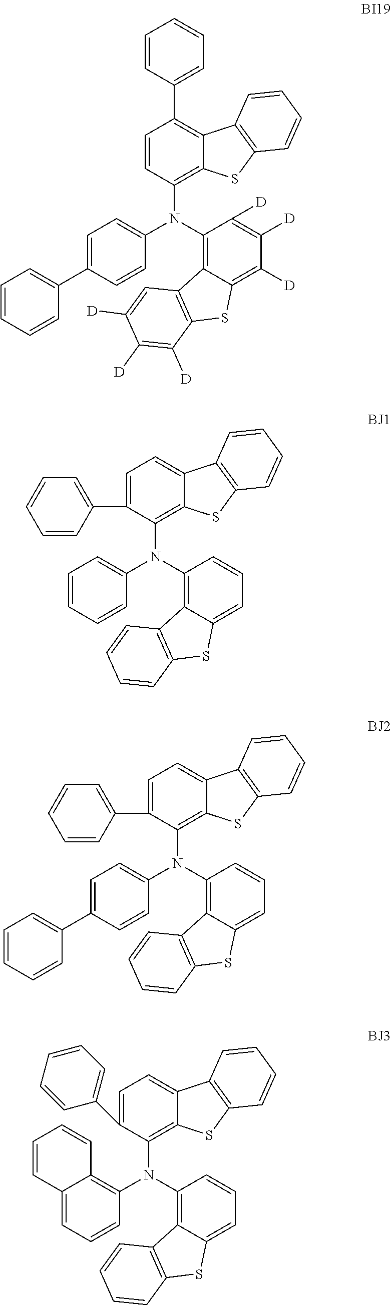 [SMILES: [2H]c1cc2c(sc3c([2H])c([2H])c([2H])c(N(c4ccc(-c5ccccc5)cc4)c4ccc(-c5ccccc5)c5c4sc4ccccc45)c32)c([2H])c1[2H] ; c1ccc(-c2ccc3c(sc4ccccc43)c2N(c2ccccc2)c2cccc3sc4ccccc4c23)cc1 ; c1ccc(-c2ccc(N(c3c(-c4ccccc4)ccc4c3sc3ccccc34)c3cccc4sc5ccccc5c34)cc2)cc1 ; c1ccc(-c2ccc3c(sc4ccccc43)c2N(c2cccc3ccccc23)c2cccc3sc4ccccc4c23)cc1]

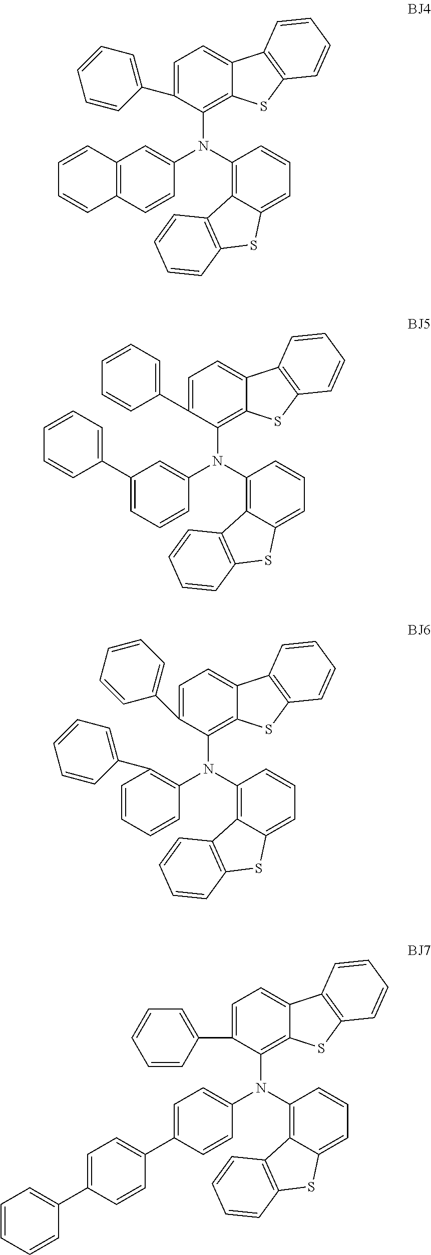 [SMILES: c1ccc(-c2ccc3c(sc4ccccc43)c2N(c2ccc3ccccc3c2)c2cccc3sc4ccccc4c23)cc1 ; c1ccc(-c2cccc(N(c3c(-c4ccccc4)ccc4c3sc3ccccc34)c3cccc4sc5ccccc5c34)c2)cc1 ; c1ccc(-c2ccccc2N(c2c(-c3ccccc3)ccc3c2sc2ccccc23)c2cccc3sc4ccccc4c23)cc1 ; c1ccc(-c2ccc(-c3ccc(N(c4c(-c5ccccc5)ccc5c4sc4ccccc45)c4cccc5sc6ccccc6c45)cc3)cc2)cc1]

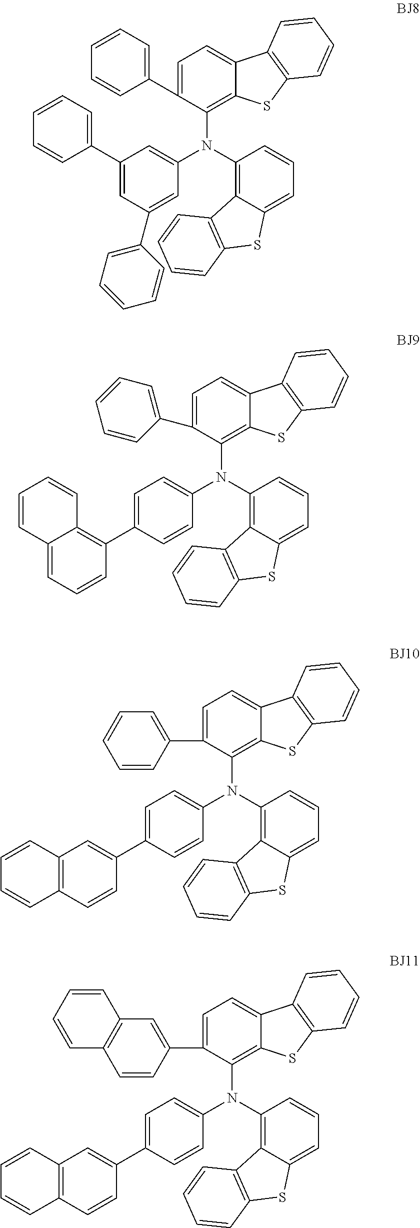 [SMILES: c1ccc(-c2cc(-c3ccccc3)cc(N(c3c(-c4ccccc4)ccc4c3sc3ccccc34)c3cccc4sc5ccccc5c34)c2)cc1 ; c1ccc(-c2ccc3c(sc4ccccc43)c2N(c2ccc(-c3cccc4ccccc34)cc2)c2cccc3sc4ccccc4c23)cc1 ; c1ccc(-c2ccc3c(sc4ccccc43)c2N(c2ccc(-c3ccc4ccccc4c3)cc2)c2cccc3sc4ccccc4c23)cc1 ; c1ccc2cc(-c3ccc(N(c4c(-c5ccc6ccccc6c5)ccc5c4sc4ccccc45)c4cccc5sc6ccccc6c45)cc3)ccc2c1]

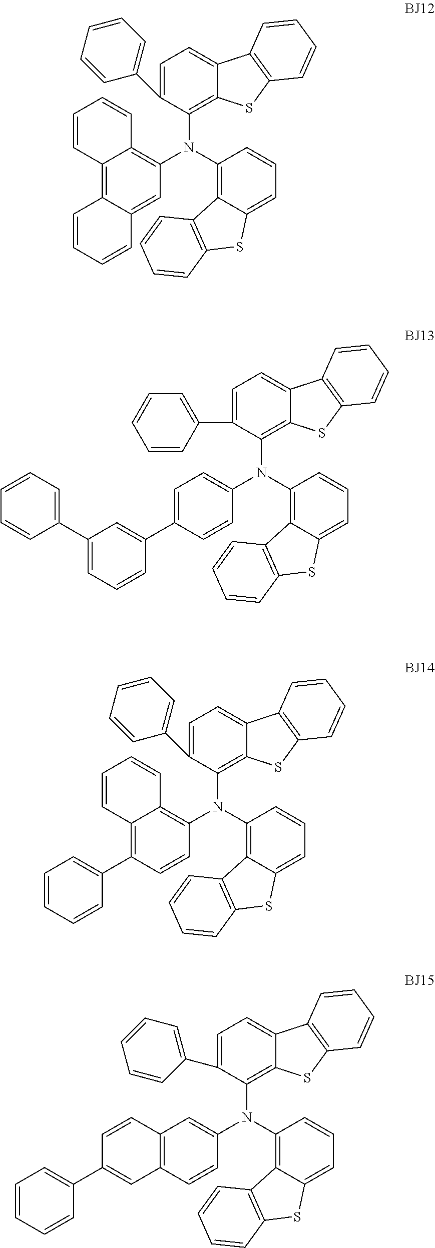 [SMILES: c1ccc(-c2ccc3c(sc4ccccc43)c2N(c2cc3ccccc3c3ccccc23)c2cccc3sc4ccccc4c23)cc1 ; c1ccc(-c2cccc(-c3ccc(N(c4c(-c5ccccc5)ccc5c4sc4ccccc45)c4cccc5sc6ccccc6c45)cc3)c2)cc1 ; c1ccc(-c2ccc3c(sc4ccccc43)c2N(c2ccc(-c3ccccc3)c3ccccc23)c2cccc3sc4ccccc4c23)cc1 ; c1ccc(-c2ccc3cc(N(c4c(-c5ccccc5)ccc5c4sc4ccccc45)c4cccc5sc6ccccc6c45)ccc3c2)cc1]

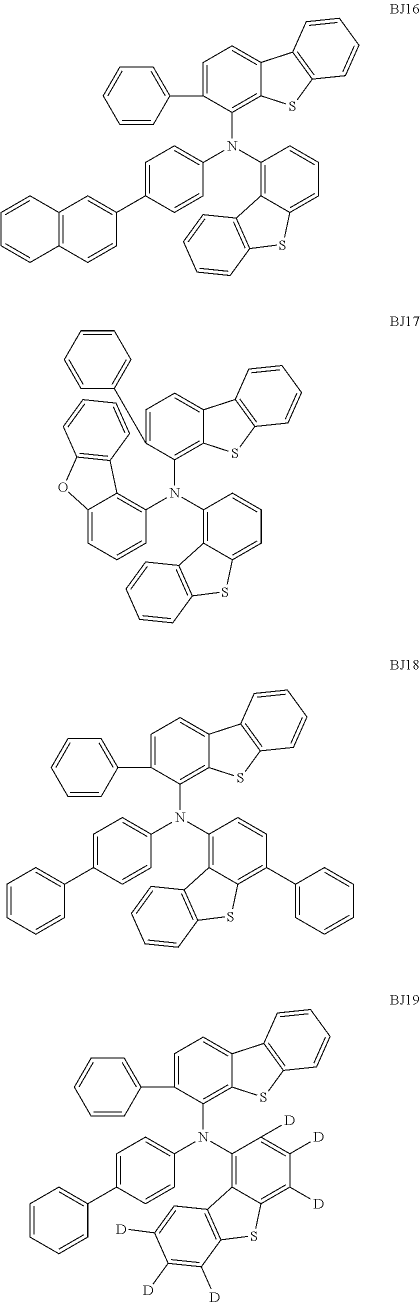 [SMILES: c1ccc(-c2ccc3c(sc4ccccc43)c2N(c2ccc(-c3ccc4ccccc4c3)cc2)c2cccc3sc4ccccc4c23)cc1 ; c1ccc(-c2ccc3c(sc4ccccc43)c2N(c2cccc3oc4ccccc4c23)c2cccc3sc4ccccc4c23)cc1 ; c1ccc(-c2ccc(N(c3c(-c4ccccc4)ccc4c3sc3ccccc34)c3ccc(-c4ccccc4)c4sc5ccccc5c34)cc2)cc1 ; [2H]c1cc2c(sc3c([2H])c([2H])c([2H])c(N(c4ccc(-c5ccccc5)cc4)c4c(-c5ccccc5)ccc5c4sc4ccccc45)c32)c([2H])c1[2H]]

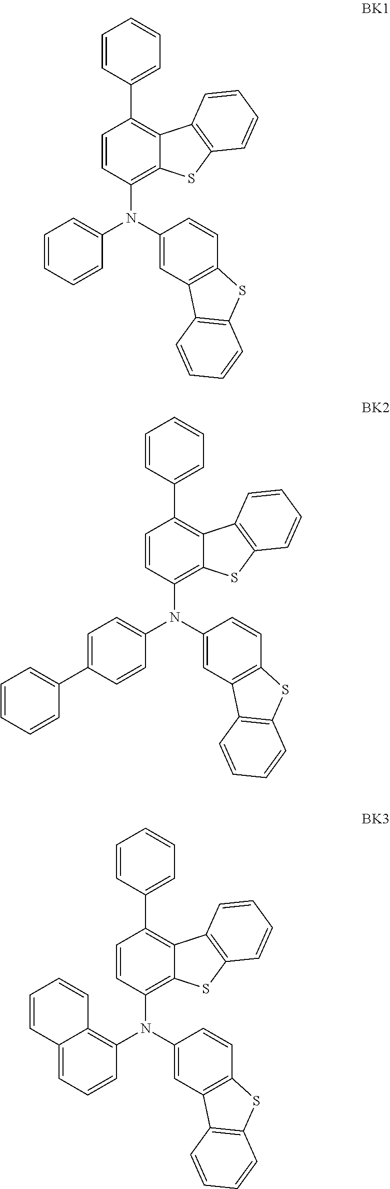

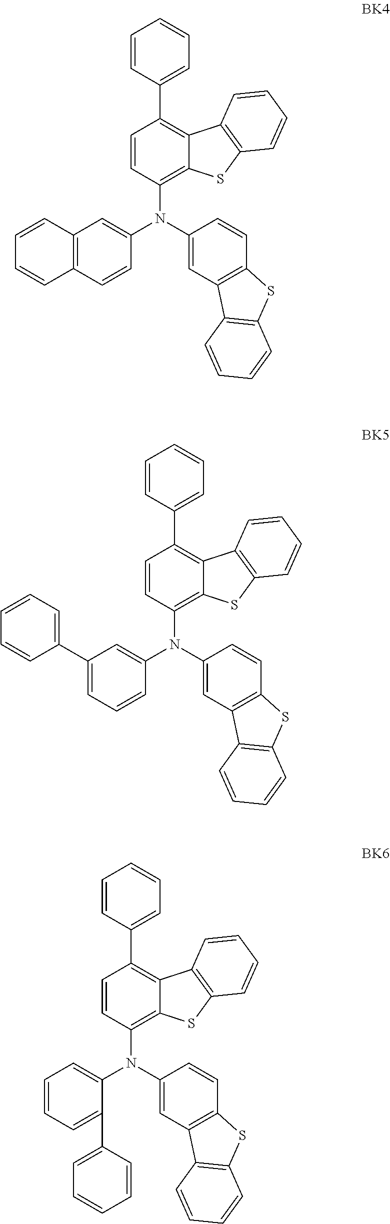 [SMILES: c1ccc(-c2ccc(N(c3ccc4ccccc4c3)c3ccc4sc5ccccc5c4c3)c3sc4ccccc4c23)cc1 ; c1ccc(-c2cccc(N(c3ccc4sc5ccccc5c4c3)c3ccc(-c4ccccc4)c4c3sc3ccccc34)c2)cc1 ; c1ccc(-c2ccccc2N(c2ccc3sc4ccccc4c3c2)c2ccc(-c3ccccc3)c3c2sc2ccccc23)cc1]

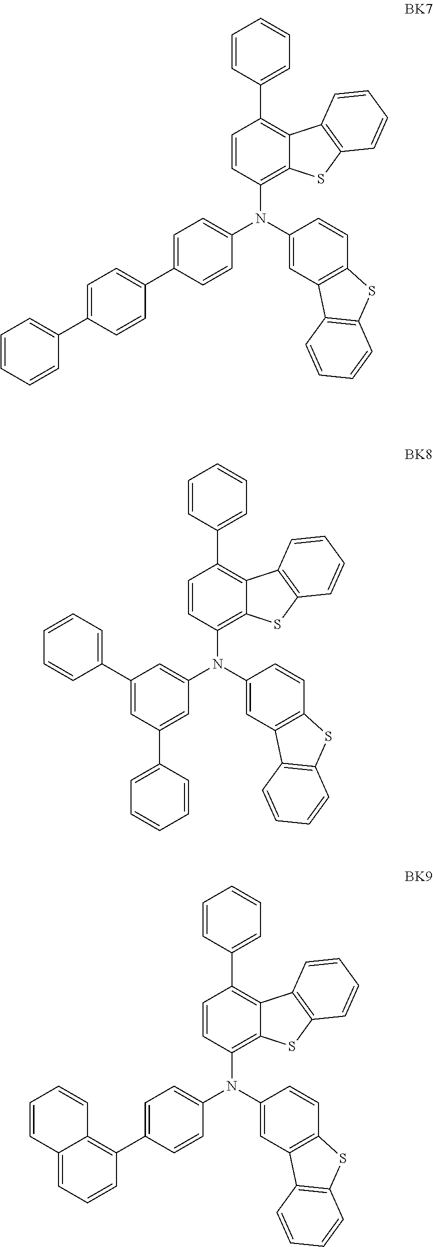 [SMILES: c1ccc(-c2ccc(-c3ccc(N(c4ccc5sc6ccccc6c5c4)c4ccc(-c5ccccc5)c5c4sc4ccccc45)cc3)cc2)cc1 ; c1ccc(-c2cc(-c3ccccc3)cc(N(c3ccc4sc5ccccc5c4c3)c3ccc(-c4ccccc4)c4c3sc3ccccc34)c2)cc1 ; c1ccc(-c2ccc(N(c3ccc(-c4cccc5ccccc45)cc3)c3ccc4sc5ccccc5c4c3)c3sc4ccccc4c23)cc1]

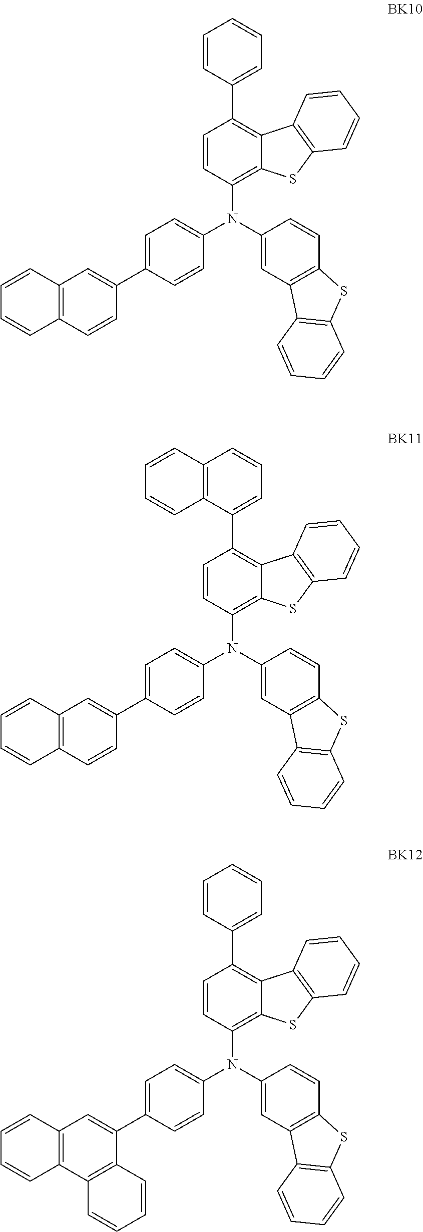 [SMILES: c1ccc(-c2ccc(N(c3ccc(-c4ccc5ccccc5c4)cc3)c3ccc4sc5ccccc5c4c3)c3sc4ccccc4c23)cc1 ; c1ccc2cc(-c3ccc(N(c4ccc5sc6ccccc6c5c4)c4ccc(-c5cccc6ccccc56)c5c4sc4ccccc45)cc3)ccc2c1 ; c1ccc(-c2ccc(N(c3ccc(-c4cc5ccccc5c5ccccc45)cc3)c3ccc4sc5ccccc5c4c3)c3sc4ccccc4c23)cc1]

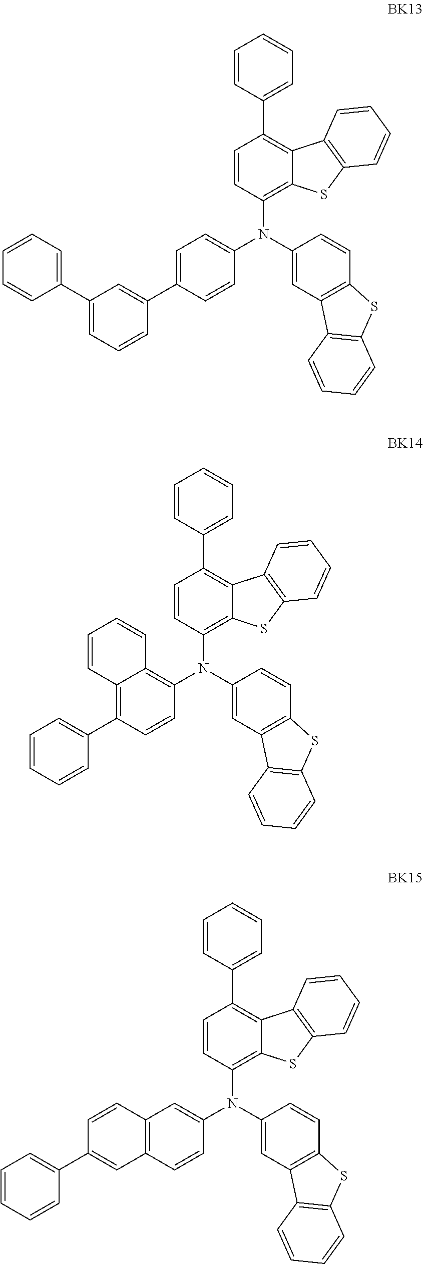 [SMILES: c1ccc(-c2cccc(-c3ccc(N(c4ccc5sc6ccccc6c5c4)c4ccc(-c5ccccc5)c5c4sc4ccccc45)cc3)c2)cc1 ; c1ccc(-c2ccc(N(c3ccc4sc5ccccc5c4c3)c3ccc(-c4ccccc4)c4c3sc3ccccc34)c3ccccc23)cc1 ; c1ccc(-c2ccc3cc(N(c4ccc5sc6ccccc6c5c4)c4ccc(-c5ccccc5)c5c4sc4ccccc45)ccc3c2)cc1]

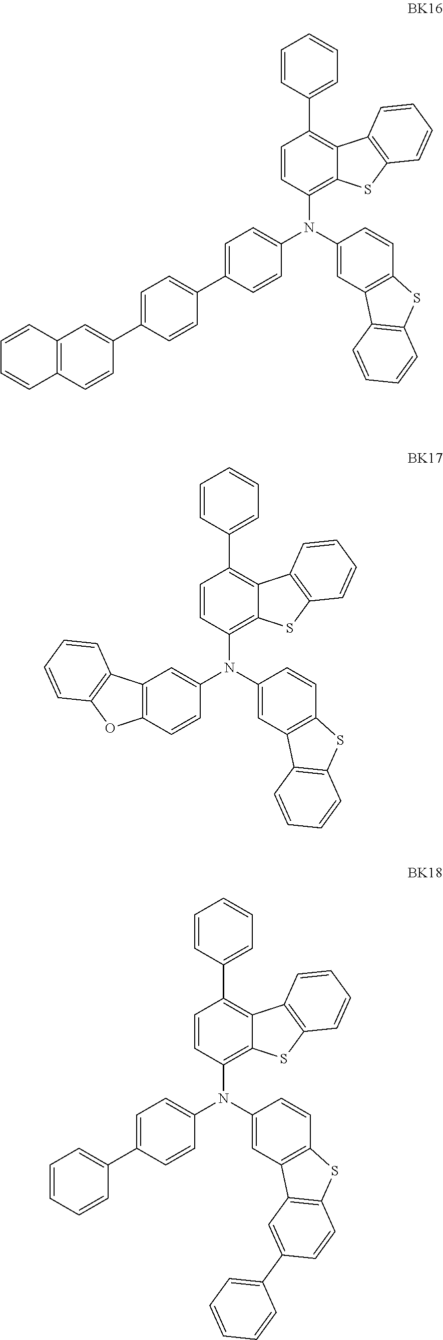 [SMILES: c1ccc(-c2ccc(N(c3ccc(-c4ccc(-c5ccc6ccccc6c5)cc4)cc3)c3ccc4sc5ccccc5c4c3)c3sc4ccccc4c23)cc1 ; c1ccc(-c2ccc(N(c3ccc4oc5ccccc5c4c3)c3ccc4sc5ccccc5c4c3)c3sc4ccccc4c23)cc1 ; c1ccc(-c2ccc(N(c3ccc4sc5ccc(-c6ccccc6)cc5c4c3)c3ccc(-c4ccccc4)c4c3sc3ccccc34)cc2)cc1]

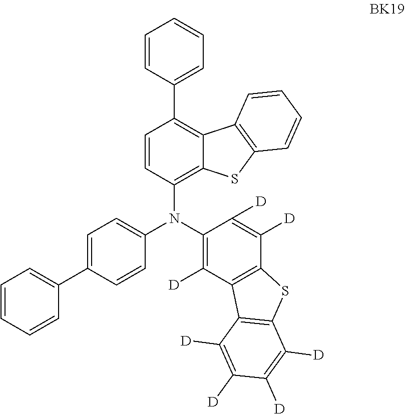 [SMILES: [2H]c1c([2H])c([2H])c2c(sc3c([2H])c([2H])c(N(c4ccc(-c5ccccc5)cc4)c4ccc(-c5ccccc5)c5c4sc4ccccc45)c([2H])c32)c1[2H]]

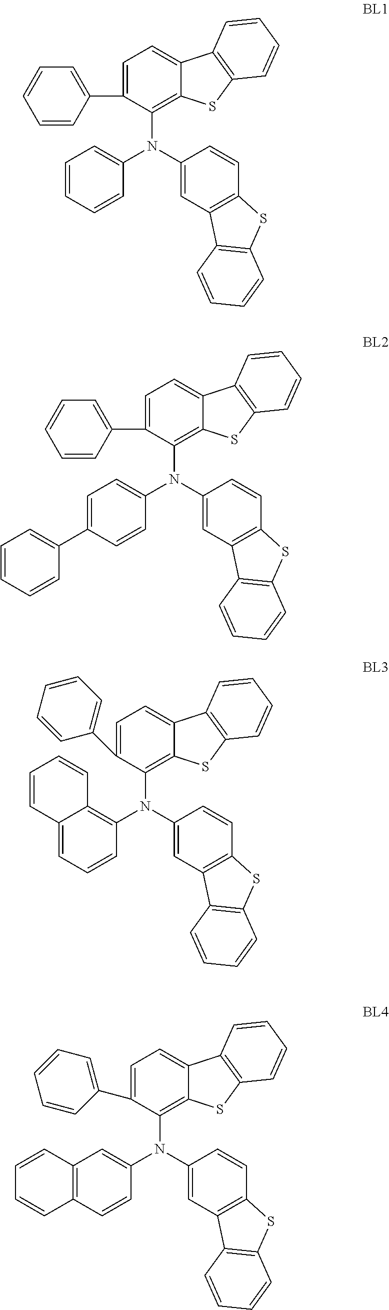

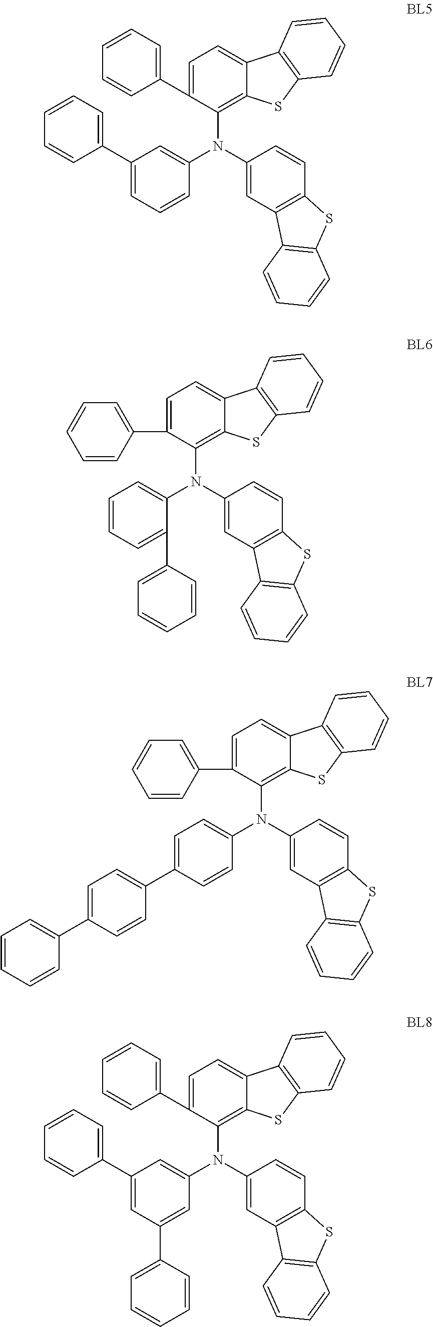 [SMILES: c1ccc(-c2cccc(N(c3ccc4sc5ccccc5c4c3)c3c(-c4ccccc4)ccc4c3sc3ccccc34)c2)cc1 ; c1ccc(-c2ccccc2N(c2ccc3sc4ccccc4c3c2)c2c(-c3ccccc3)ccc3c2sc2ccccc23)cc1 ; c1ccc(-c2ccc(-c3ccc(N(c4ccc5sc6ccccc6c5c4)c4c(-c5ccccc5)ccc5c4sc4ccccc45)cc3)cc2)cc1 ; c1ccc(-c2cc(-c3ccccc3)cc(N(c3ccc4sc5ccccc5c4c3)c3c(-c4ccccc4)ccc4c3sc3ccccc34)c2)cc1]

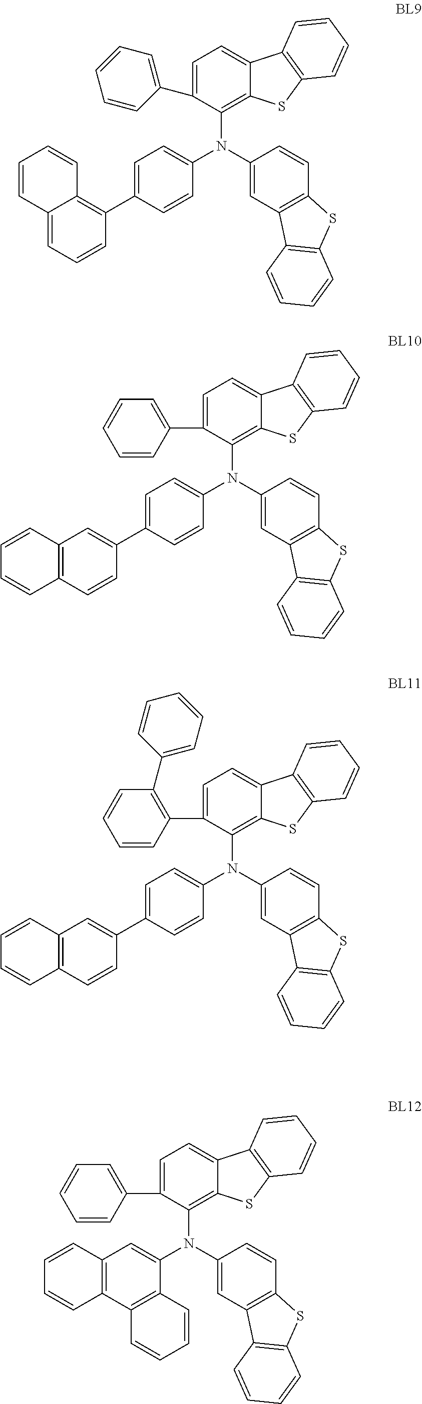 [SMILES: c1ccc(-c2ccc3c(sc4ccccc43)c2N(c2ccc(-c3cccc4ccccc34)cc2)c2ccc3sc4ccccc4c3c2)cc1 ; c1ccc(-c2ccc3c(sc4ccccc43)c2N(c2ccc(-c3ccc4ccccc4c3)cc2)c2ccc3sc4ccccc4c3c2)cc1 ; c1ccc(-c2ccccc2-c2ccc3c(sc4ccccc43)c2N(c2ccc(-c3ccc4ccccc4c3)cc2)c2ccc3sc4ccccc4c3c2)cc1 ; c1ccc(-c2ccc3c(sc4ccccc43)c2N(c2ccc3sc4ccccc4c3c2)c2cc3ccccc3c3ccccc23)cc1]

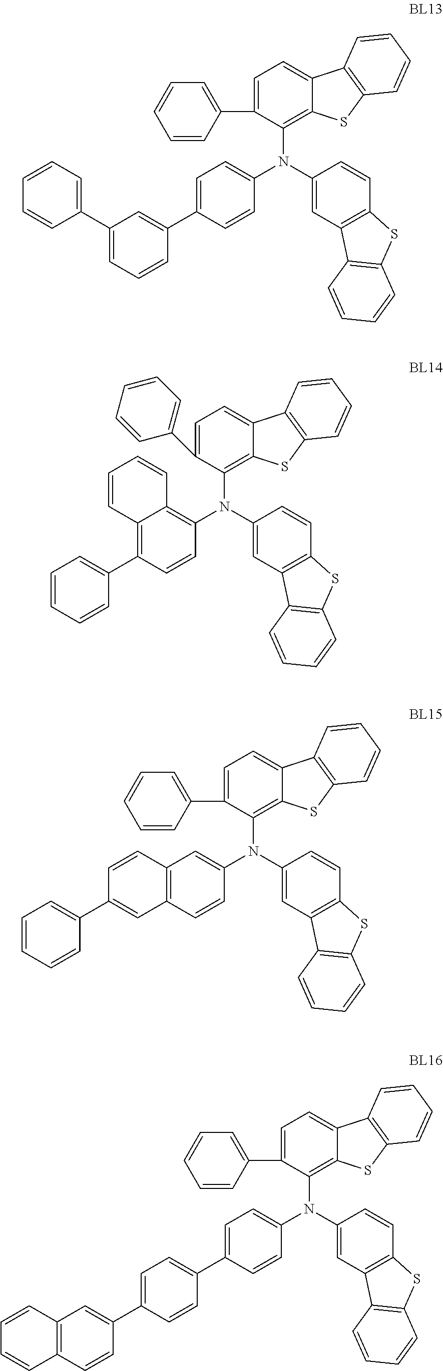 [SMILES: c1ccc(-c2cccc(-c3ccc(N(c4ccc5sc6ccccc6c5c4)c4c(-c5ccccc5)ccc5c4sc4ccccc45)cc3)c2)cc1 ; c1ccc(-c2ccc3c(sc4ccccc43)c2N(c2ccc3sc4ccccc4c3c2)c2ccc(-c3ccccc3)c3ccccc23)cc1 ; c1ccc(-c2ccc3cc(N(c4ccc5sc6ccccc6c5c4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc3sc4ccccc4c3c2)cc1]

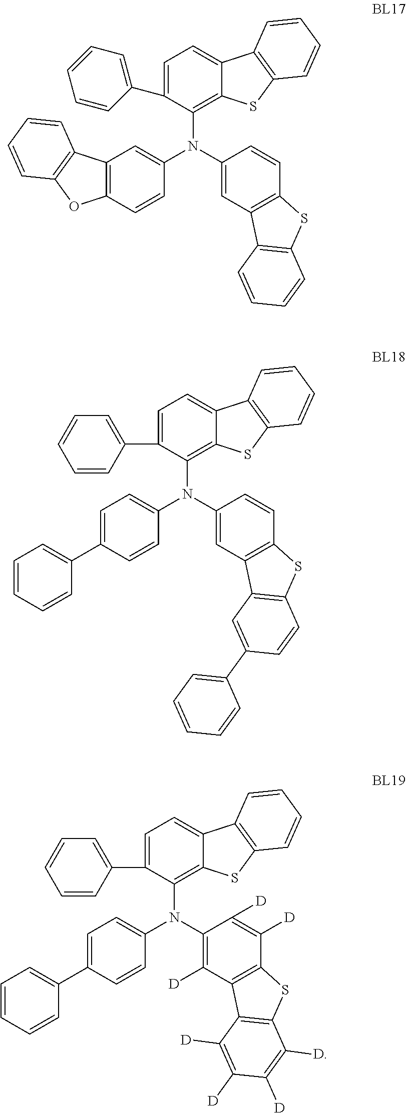

In the embodiment compounds presented in Compound Group 1, “D” corresponds to a deuterium atom.

The amine compound according to an embodiment includes the first substituent, the second substituent, and the third substituent linked to the core nitrogen atom, thereby achieving high efficiency, low voltage, high brightness, and a long service life of the light emitting element.

The amine compound of an embodiment may include an amine group, and the first to third substituents have a structure bonded to the amine group of the amine compound of an embodiment. In this case, each of the first substituent and the second substituent necessarily includes a dibenzoheterol moiety. The first substituent is directly bonded to the core nitrogen atom at the fourth carbon position, and an aryl group or a heteroaryl group is necessarily substituted at the first or third carbon position of the first substituent. The second substituent is linked to the core nitrogen atom via an arylene linker or a heteroarylene linker, or directly linked to the core nitrogen atom without a separate linker. The third substituent is an aryl group or a heteroaryl group directly linked to the core nitrogen atom.

In the amine compound of an embodiment having such a structure includes the first substituent which is linked to the core nitrogen atom at the fourth carbon position and has a dibenzoheterol structure, so that an oxygen atom and a nitrogen atom may be bonded at the ortho-position, thereby the charge density may increase, and thus the hole transport ability may be improved, leading to the improvement of the efficiency. In addition, the first substituent is substituted with an aryl group or a heteroaryl group at the first or third carbon position, and thus the conjugated structure may be increased, thereby the stability of the molecular structure may be improved, and thus the element service life may be improved when the first substituent is applied to the light emitting element. In addition, the first substituent is substituted with an aryl group or a heteroaryl group at the first or third carbon position, and thus intermolecular stacking is likely to occur, thereby the distance between the molecules may narrow, and thus the hole transport ability may be improved. The amine compound of an embodiment further includes the second substituent having a dibenzoheterol structure, and thus the hole transport ability may further be improved, and the stability in a radical cation state may be improved. Therefore, when the amine compound according to an embodiment of the present disclosure is applied to the hole transport region HTR of the light emitting element ED, the light emitting element may achieve high efficiency, a low voltage, high brightness, and a long service life.

When the amine compound of an embodiment is used in the hole transport region, the light extraction mode may be changed between the first electrode and the second electrode, and thus the external quantum efficiency may be increased. Accordingly, when the amine compound of an embodiment is used in the hole transport region, the luminous efficiency of the light emitting element may be increased and the service life of the light emitting element may be improved.

The hole transport region HTR in the light emitting element ED of an embodiment may further include a compound represented by Formula H-1 below:

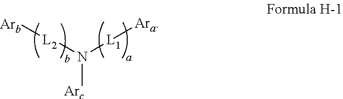

In Formula H-1 above, L₁ and L₂ may be each independently a direct linkage, a substituted or unsubstituted arylene group having 6 to 30 ring-forming carbon atoms, or a substituted or unsubstituted heteroarylene group having 2 to 30 ring-forming carbon atoms. A and b may be each independently an integer of 0 to 10. In some embodiments, when a or b is an integer of 2 or greater, a plurality of L₁'s and L₂'s may be each independently a substituted or unsubstituted arylene group having 6 to 30 ring-forming carbon atoms, or a substituted or unsubstituted heteroarylene group having 2 to 30 ring-forming carbon atoms.

In Formula H-1, Arₐ and Ar_{b} may be each independently a substituted or unsubstituted aryl group having 6 to 30 ring-forming carbon atoms, or a substituted or unsubstituted heteroaryl group having 2 to 30 ring-forming carbon atoms. In addition, in Formula H-1, Ar_{c} may be a substituted or unsubstituted aryl group having 6 to 30 ring-forming carbon atoms.

The compound represented by Formula H-1 above may be a monoamine compound. In some embodiments, the compound represented by Formula H-1 above may be a diamine compound in which at least one selected from among Arₐ to Ar_{c} contains an amine group as a substituent. In addition, the compound represented by Formula H-1 above may be a carbazole-based compound containing a substituted or unsubstituted carbazole group in at least one selected from Arₐ and Ar_{b}, or a fluorene-based compound containing a substituted or unsubstituted fluorene group in at least one selected from Arₐ and Ar_{b}.

The compound represented by Formula H-1 may be represented by any one selected from among the compounds of Compound Group H below. However, the compounds listed in Compound Group H below are examples, and the compounds represented by Formula H-1 are not limited to those represented by Compound Group H below:

### Compound Group H

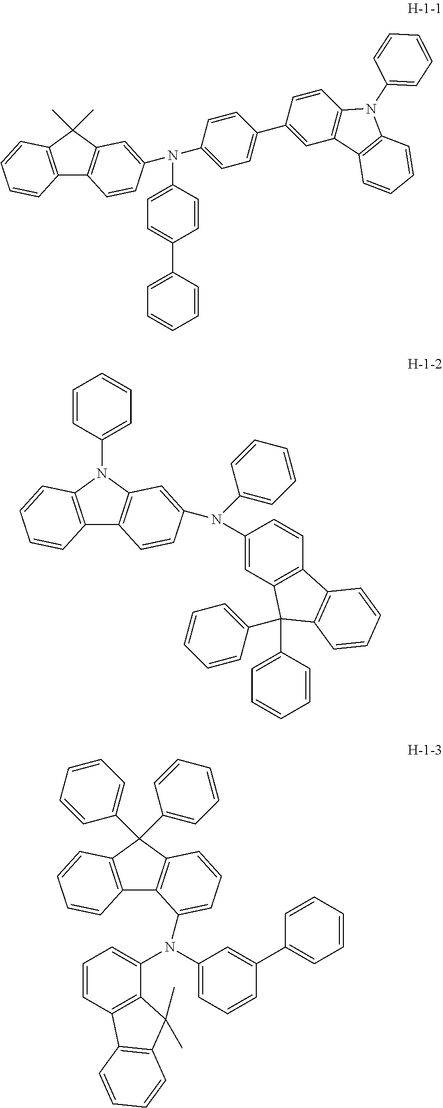 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

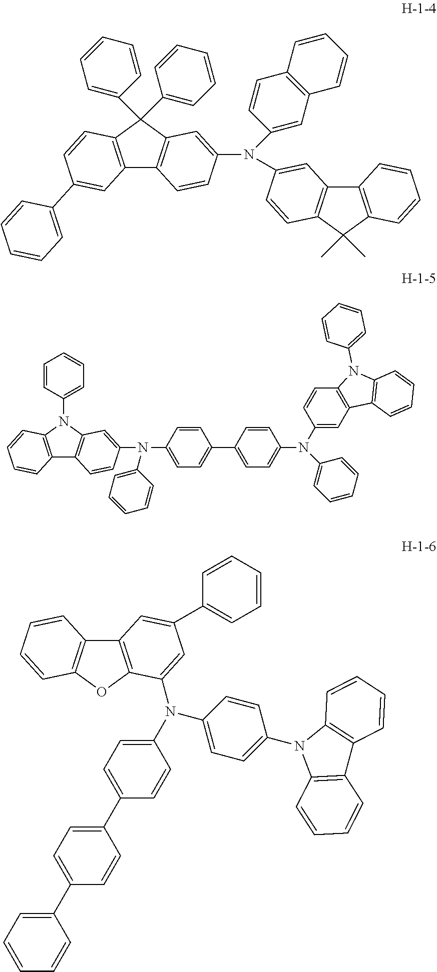 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

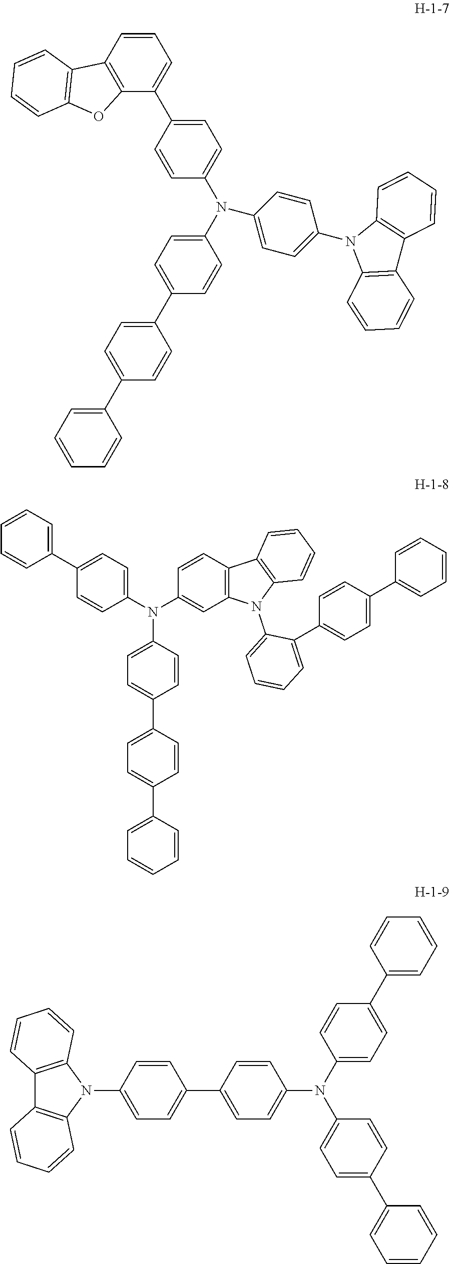 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

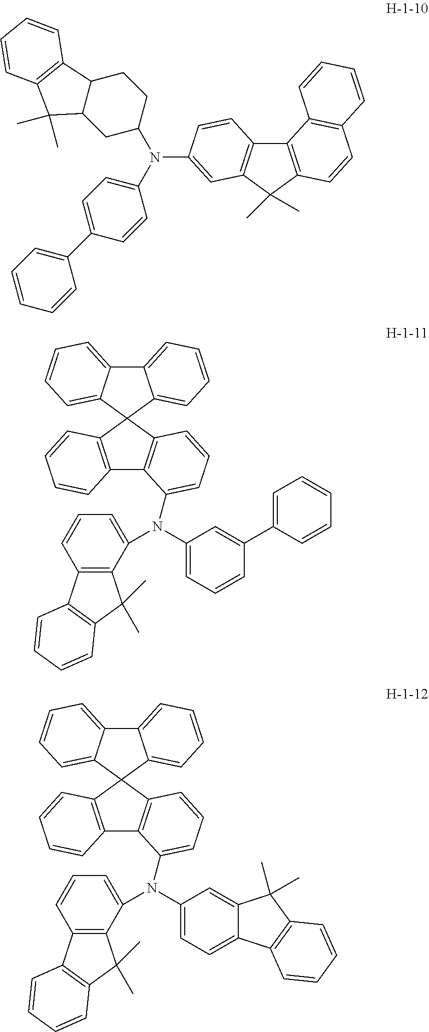

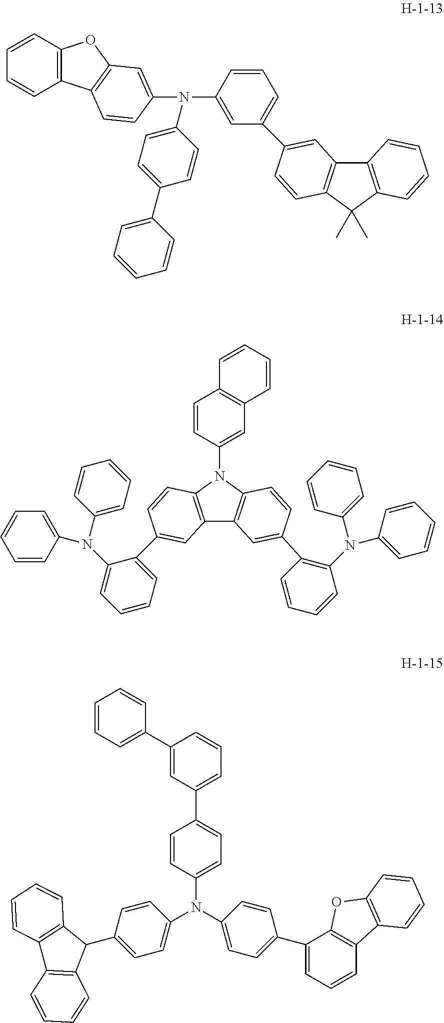 [SMILES: CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(C5c6ccccc6-c6ccccc65)cc4)cc3)c2)cc1]

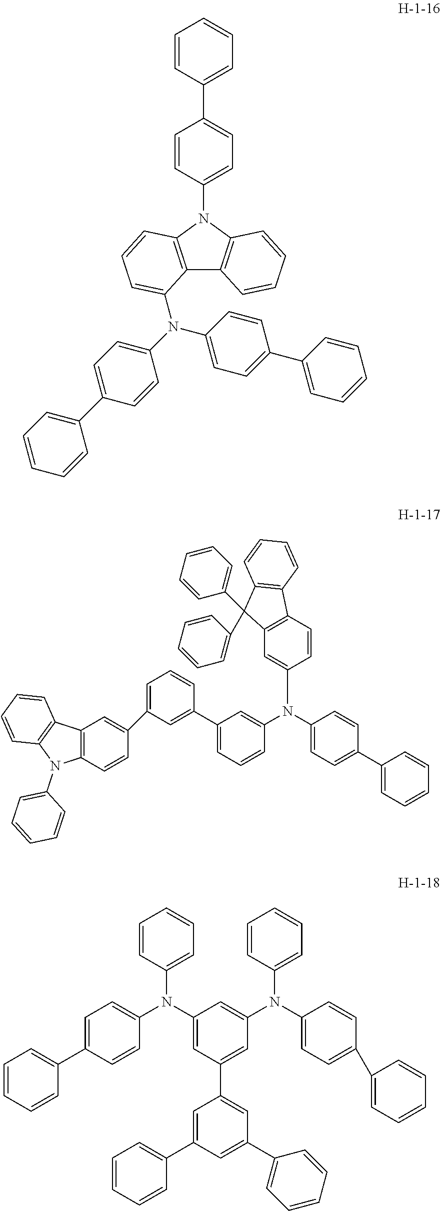 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

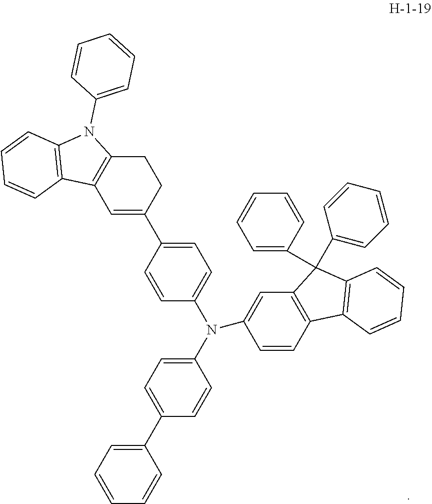 [SMILES: C1=C(c2ccc(N(c3ccc(-c4ccccc4)cc3)c3ccc4c(c3)C(c3ccccc3)(c3ccccc3)c3ccccc3-4)cc2)CCc2c1c1ccccc1n2-c1ccccc1]

In addition, the hole transport region HTR may further include any suitable hole transport material generally used in the art.

For example, the hole transport region HTR may include a phthalocyanine compound such as copper phthalocyanine; N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-I-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include a carbazole-based derivative such as N-phenyl carbazole or polyvinyl carbazole, a fluorene-based derivative, a triphenyl amine-based derivative such as 4,4′,4″-tris(N-carbazolyl)triphenylamine (TCTA), or N,N′-bis(3-methylphenyl)-N,N′-diphenyl-[1,1′-biphenyl]-4,4′-diamine (TPD), N,N′-di(naphthalene-I-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

In addition, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one selected from a hole injection layer HIL, a hole transport layer HTL, and an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, when the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the above-described ranges, suitable or satisfactory hole transport properties may be achieved without a substantial increase in driving voltage.

The hole transport region HTR may further include a charge generating material to increase conductivity (e.g., electrical conductivity) in addition to the above-described materials. The charge generating material may be dispersed uniformly or non-uniformly in the hole transport region HTR. The charge generating material may be, for example, a p-dopant. The p-dopant may include at least one selected from a halogenated metal compound, a quinone derivative, a metal oxide, and a cyano group-containing compound, but embodiments of the present disclosure are not limited thereto. For example, the p-dopant may include a metal halide compound such as CuI or RbI, a quinone derivative such as tetracyanoquinodimethane (TCNQ) or 2,3,5,6-tetrafluoro-7,7,8,8-tetracyanoquinodimethane (F4-TCNQ), a metal oxide such as tungsten oxide or molybdenum oxide, a cyano group-containing compound such as dipyrazino[2,3-f: 2′,3′-h] quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments of the present disclosure are not limited thereto.

As described above, the hole transport region HTR may further include a buffer layer in addition to the hole injection layer HIL, the hole transport layer HTL, and the electron blocking layer EBL. The buffer layer may compensate for a resonance distance according to the wavelength of light emitted from the emission layer EML and may thus increase light emission efficiency. A material that may be included in the hole transport region HTR may be used as a material to be included in the buffer layer.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

The emission layer EML in the light emitting element ED of an embodiment may emit blue light. The light emitting element ED of an embodiment may include the above-described amine compound of an embodiment in the hole transport region HTR, thereby exhibiting high efficiency and long service life characteristics in the blue light emitting region. However, embodiments of the present disclosure are not limited thereto.

In the light emitting element ED of an embodiment, the emission layer EML may include an anthracene derivative, a pyrene derivative, a fluoranthene derivative, a chrysene derivative, a dihydrobenzanthracene derivative, and/or a triphenylene derivative. For example, the emission layer EML may include the anthracene derivative and/or the pyrene derivative.

In each of the light emitting elements ED of embodiments illustrated in FIGS. 3 to 6, the emission layer EML may include a host and a dopant, and the emission layer EML may include a compound represented by Formula E-1 below. The compound represented by Formula E-1 below may be used as a fluorescent host material.

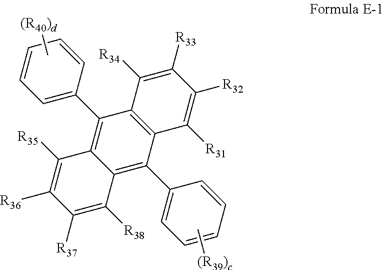

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In some embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may be each independently an integer of 0 to 5.

Formula E-1 may be represented by any one selected from among Compound E1 to Compound E19 below:

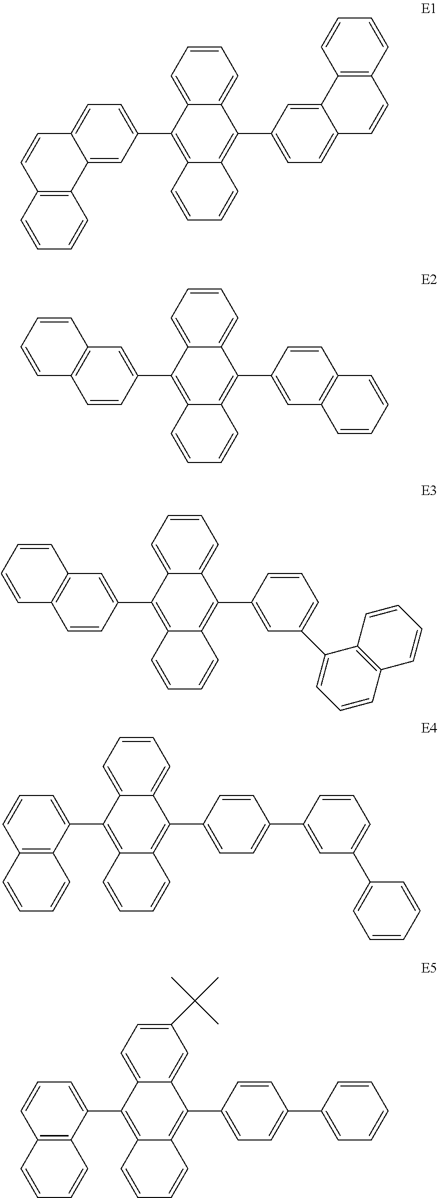

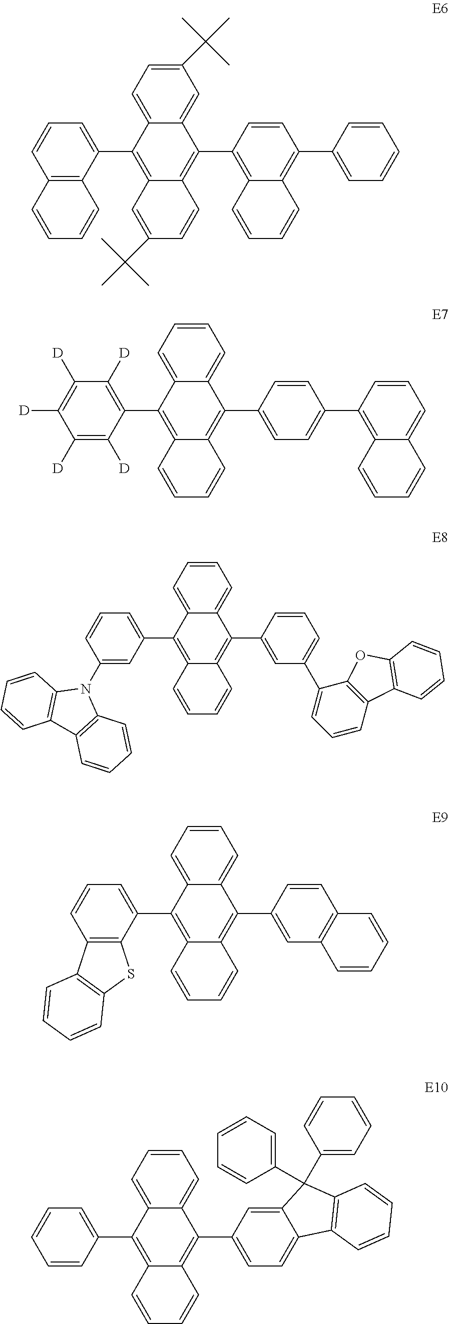

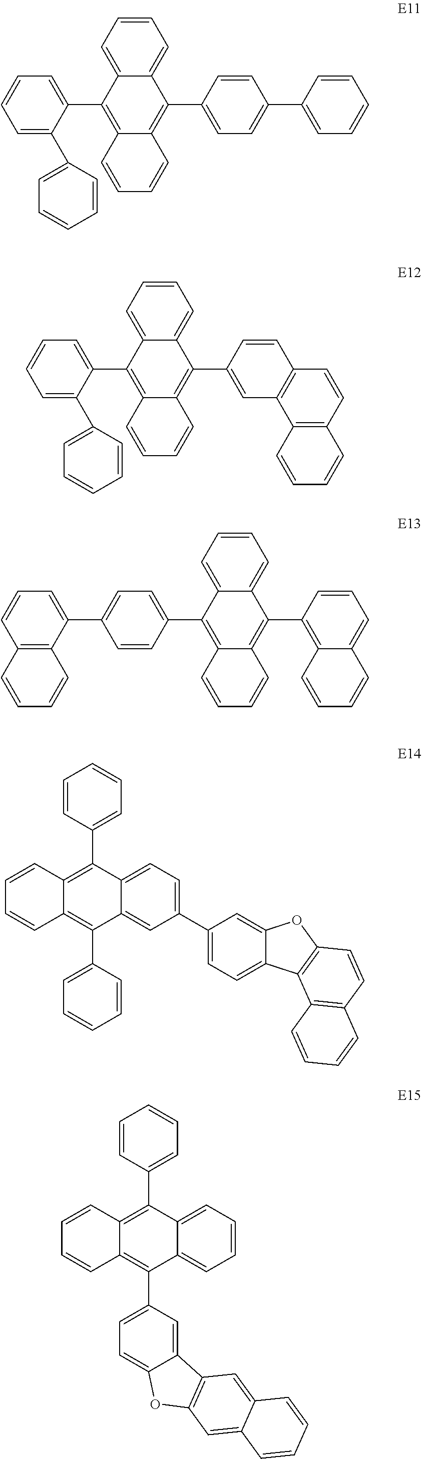

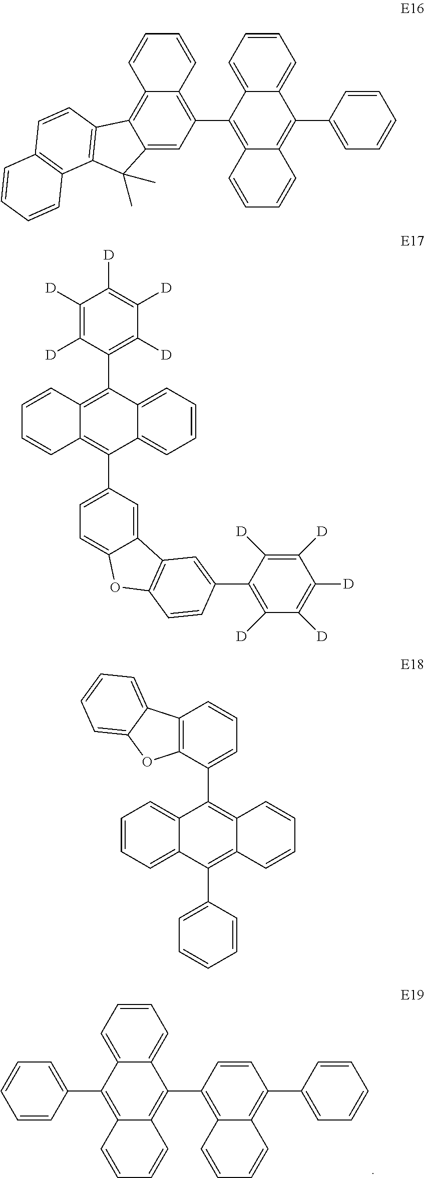

In an embodiment, the emission layer EML may include a compound represented by Formula E-2a or Formula E-2b below. The compound represented by Formula E-2a or Formula E-2b below may be used as a phosphorescent host material.

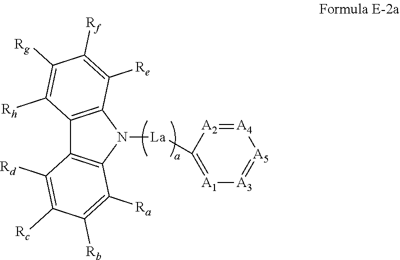

In Formula E-2a, and a may be an integer of 0 to 10, Lₐ may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or more, a plurality of Lₐ's may be each independently a substituted or unsubstituted arylene group having 6 to 30 ring-forming carbon atoms, or a substituted or unsubstituted heteroarylene group having 2 to 30 ring-forming carbon atoms.

In addition, in Formula E-2a, A₁ to A₅ may be each independently N or CRI. Rₐ to R₁ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to R₁ may be bonded to an adjacent group to form a hydrocarbon ring or a heterocycle containing N, O, S, etc., as a ring-forming atom.

In some embodiments, in Formula E-2a, two or three selected from among A₁ to A₅ may be N, and the rest may be CRᵢ.

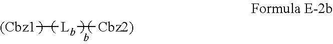

In Formula E-2b, Cbz1 and Cbz2 may be each independently an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. In some embodiments, b is an integer of 0 to 10, and when b is an integer of 2 or more, a plurality of L_{b}'s may be each independently a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among the compounds of Compound Group E-2 below. However, the compounds listed in Compound Group E-2 below are examples, and the compound represented by Formula E-2a or Formula E-2b is not limited to those represented in Compound Group E-2 below.

### Compound Group E-2

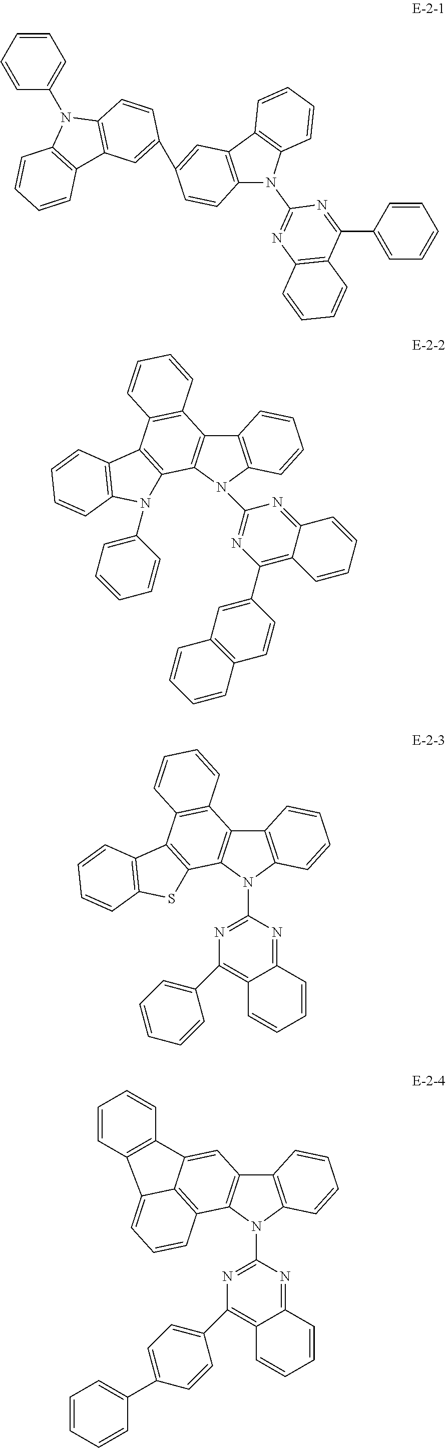

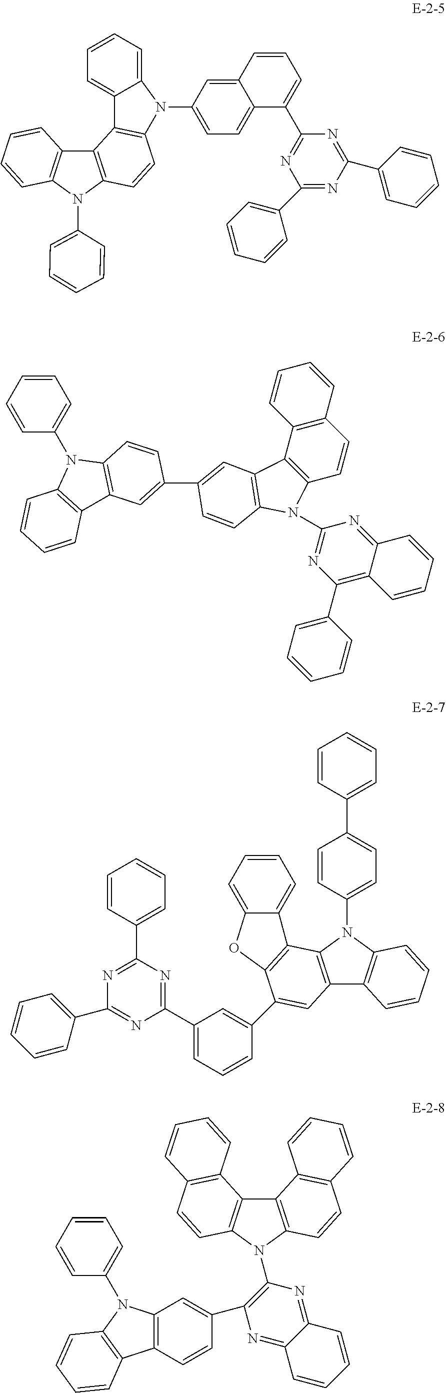

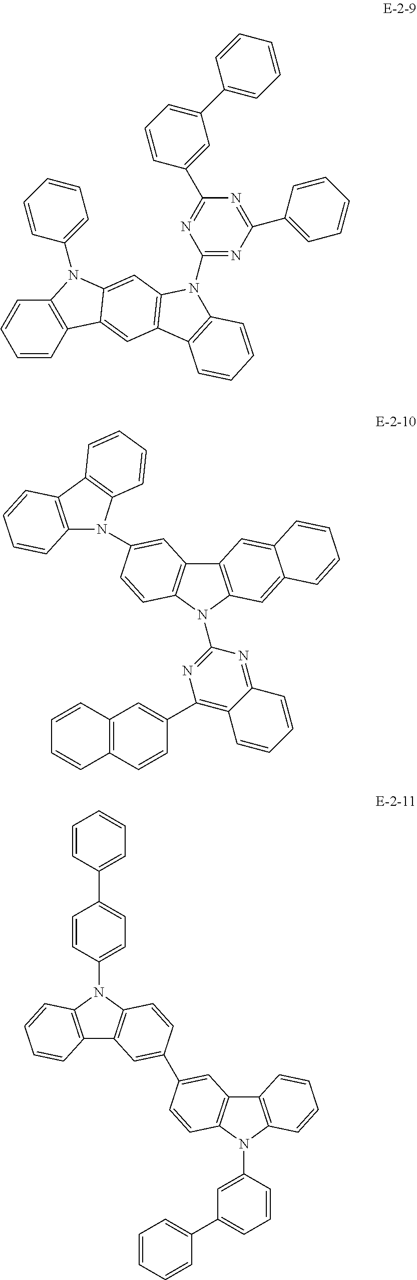

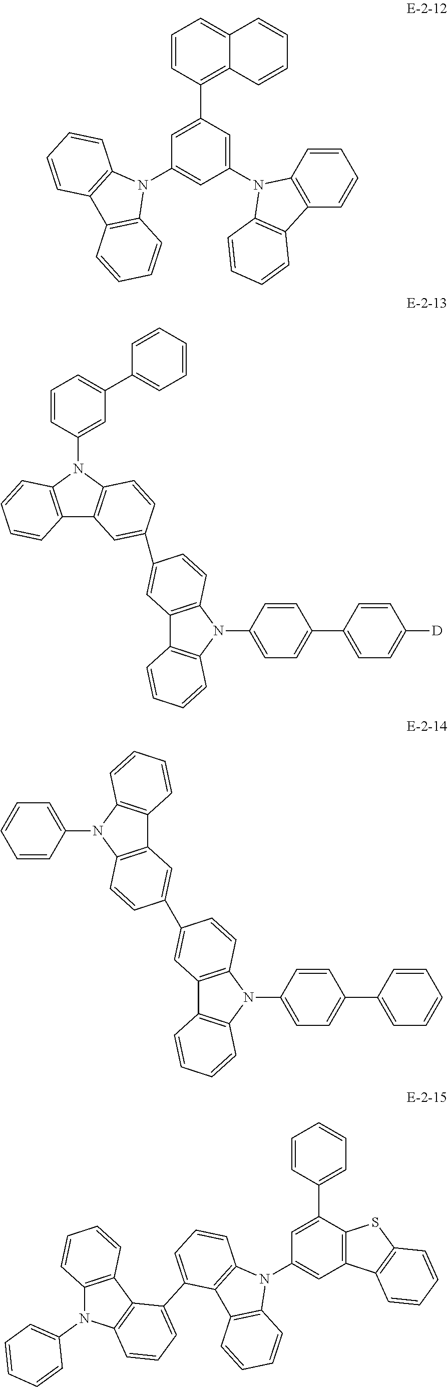 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

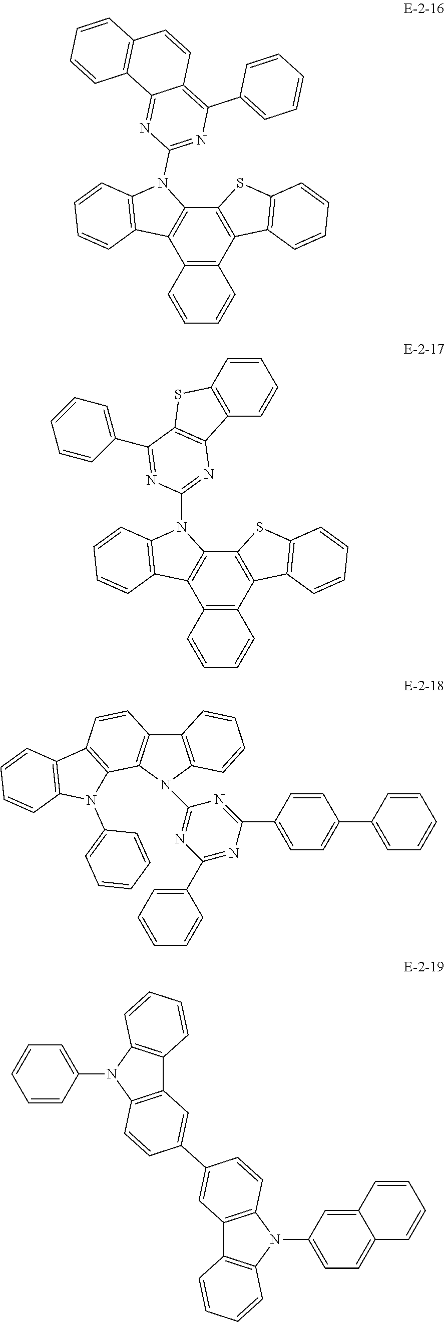

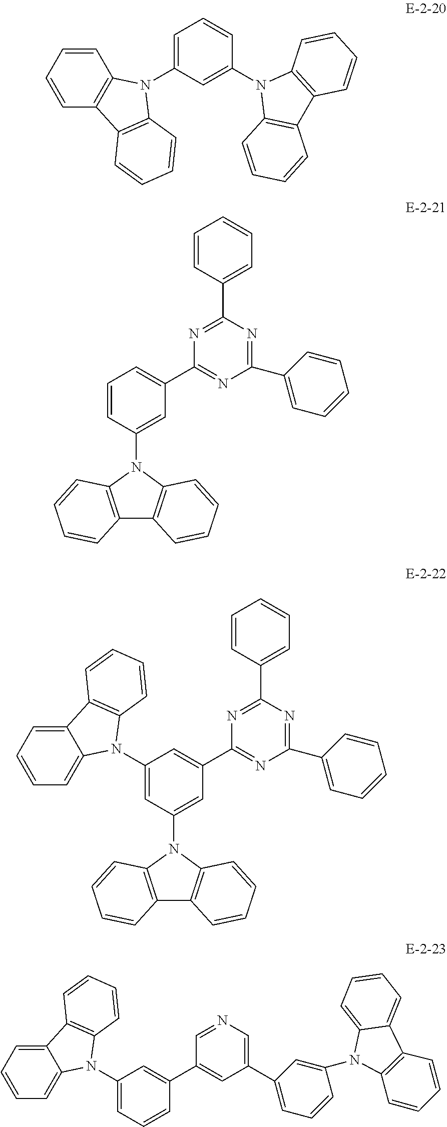 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

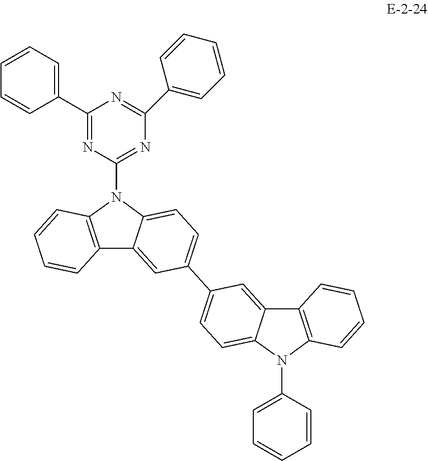 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include any suitable material generally used in the art as a host material. For example, the emission layer EML may include, as a host material, at least one selected from bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and 1,3,5-tris(1-phenyl-1H-benzo[d]imidazole-2-yl)benzene (TPBi). However, embodiments of the present disclosure are not limited thereto, for example, tris(8-hydroxyquinolino)aluminum (Alq₃), 9,10-di(naphthalene-2-yl)anthracene (I), 2-tert-butyl-9,10-di(naphth-2-yl)anthracene (TBADN), distyrylarylene (DSA), ‘,4’-bis(9-carbazolyl)-‘,2’-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as a host material.

The emission layer EML may include a compound represented by Formula M-a or Formula M-b below. The compound represented by Formula M-a or Formula M-b below may be used as a phosphorescent dopant material. In addition, the compound represented by Formula M-a or Formula M-b in an embodiment may be used as an auxiliary dopant material.

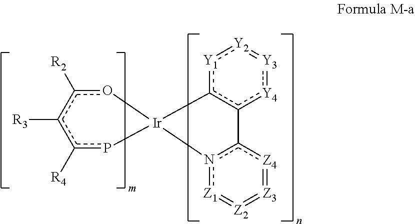

In Formula M-a above, Y₁ to Y₄ and Z₁ to Z₄ may be each independently CR₁ or N, R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m is 0 or 1, and n is 2 or 3. In Formula M-a, when m is 0, n is 3, and when m is 1, n is 2.

The compound represented by Formula M-a may be used as a phosphorescent dopant.

The compound represented by Formula M-a may be represented by any one selected from among Compound M-a1 to Compound M-a25 below. However, Compounds M-a1 to M-a25 below are examples, and the compound represented by Formula M-a is not limited to those represented by Compounds M-a1 to M-a25 below.

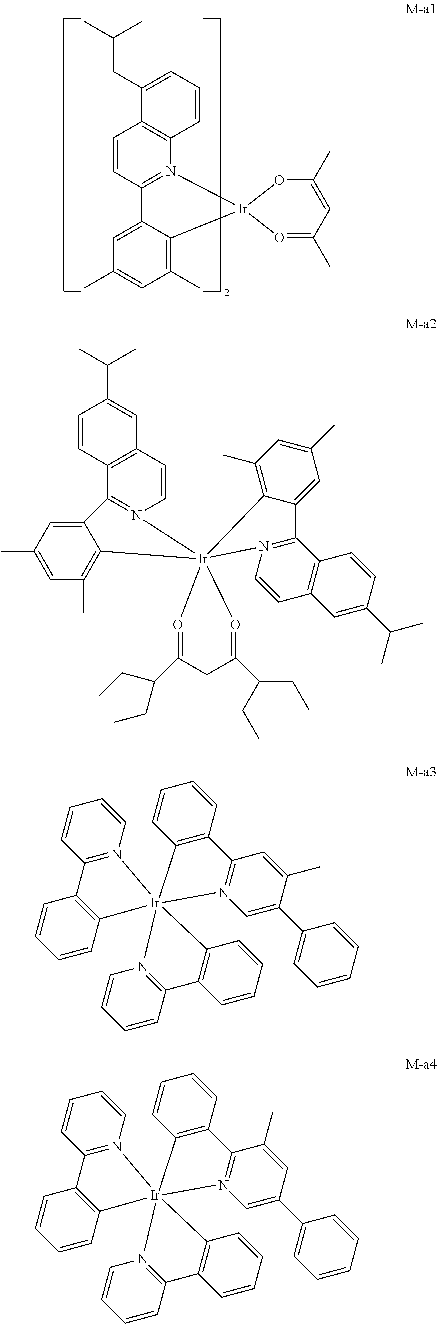

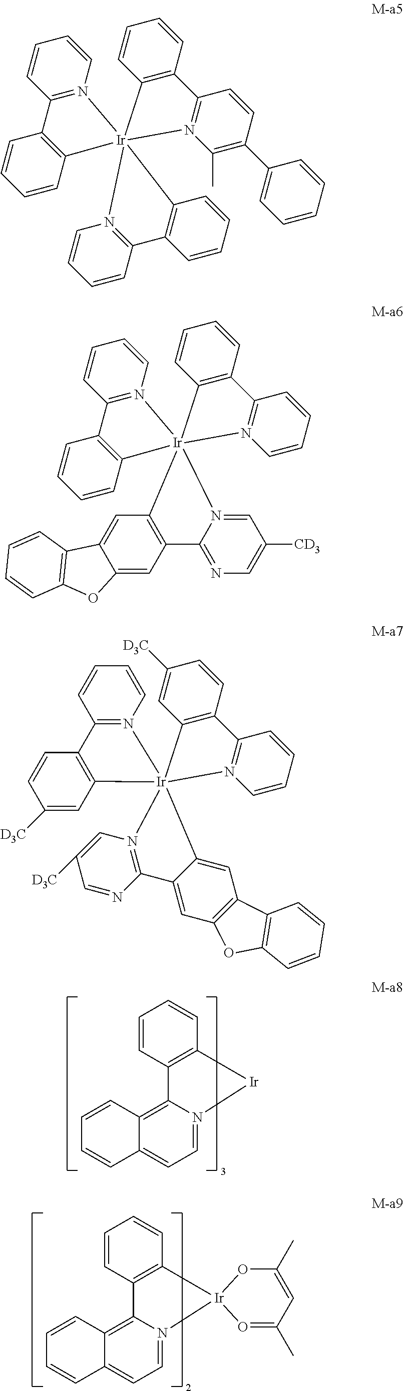

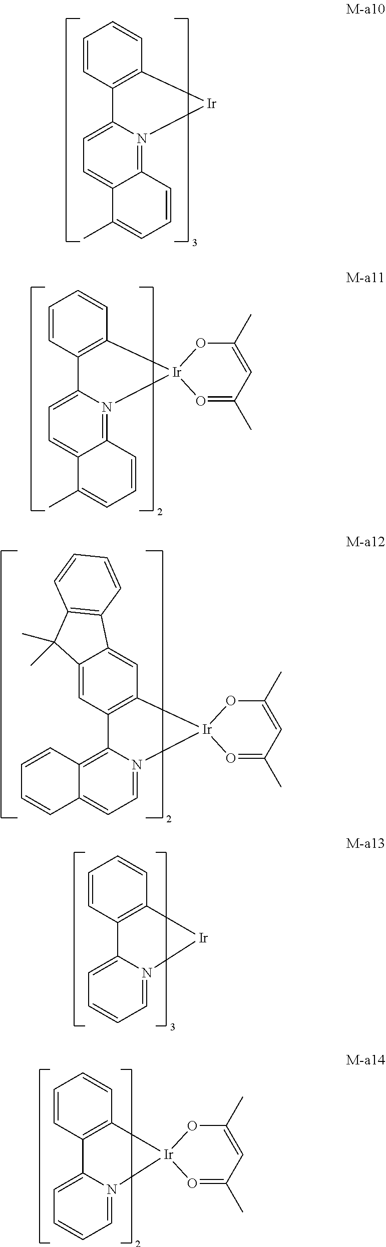

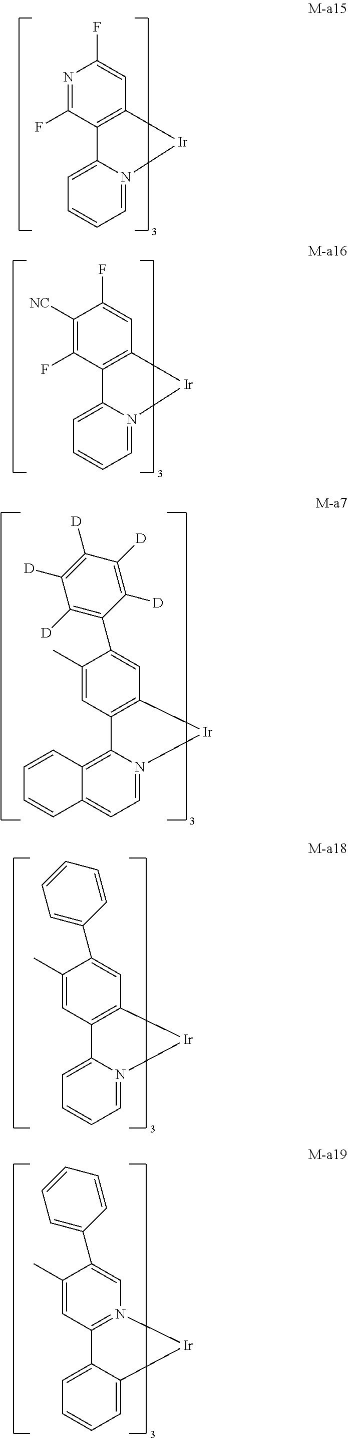

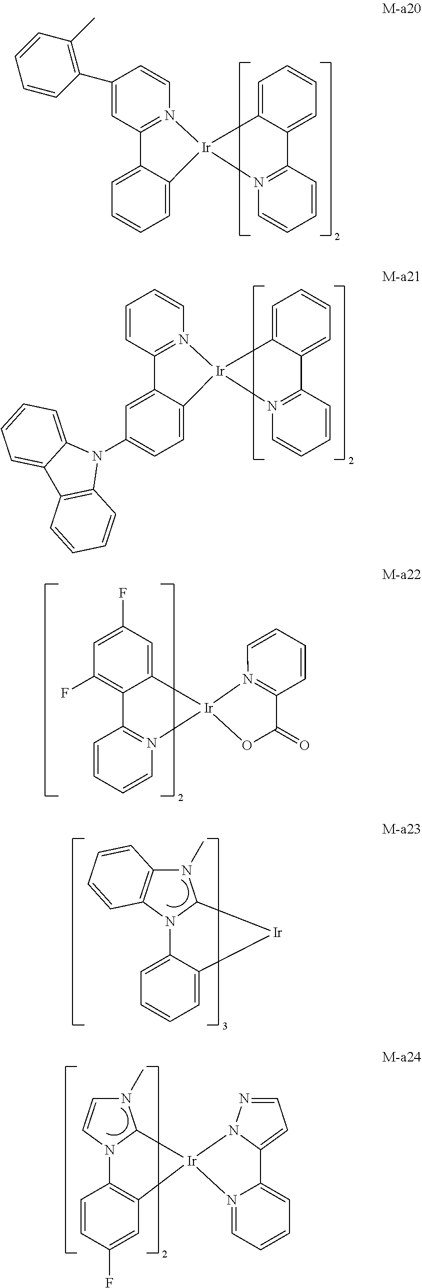

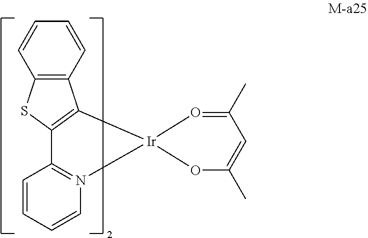

Compound M-a1 and Compound M-a2 may be used as a red dopant material, and Compound M-a3 to Compound M-a7 may be used as a green dopant material.

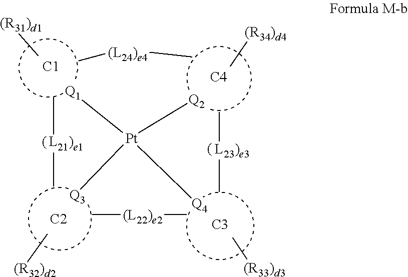

In Formula M-b, Q₁ to Q₄ are each independently C or N, and C1 to C4 are each independently a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ are each independently a direct linkage,

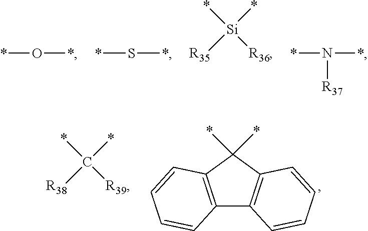

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 are each independently 0 or 1. R₃₁ to R₃₉ are each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and d1 to d4 are each independently an integer of 0 to 4.

The compound represented by Formula M-b may be used as a blue phosphorescent dopant or a green phosphorescent dopant. In addition, the compound represented by Formula M-b may be further included as an auxiliary dopant in the emission layer EML in an embodiment.

The compound represented by Formula M-b may be represented by any one selected from among Compound M-b-1 to Compound M-b-11 below. However, the following compounds are examples, and the compounds represented by Formula M-b are not limited to Compounds M-b-1 to Compound M-b-11 below:

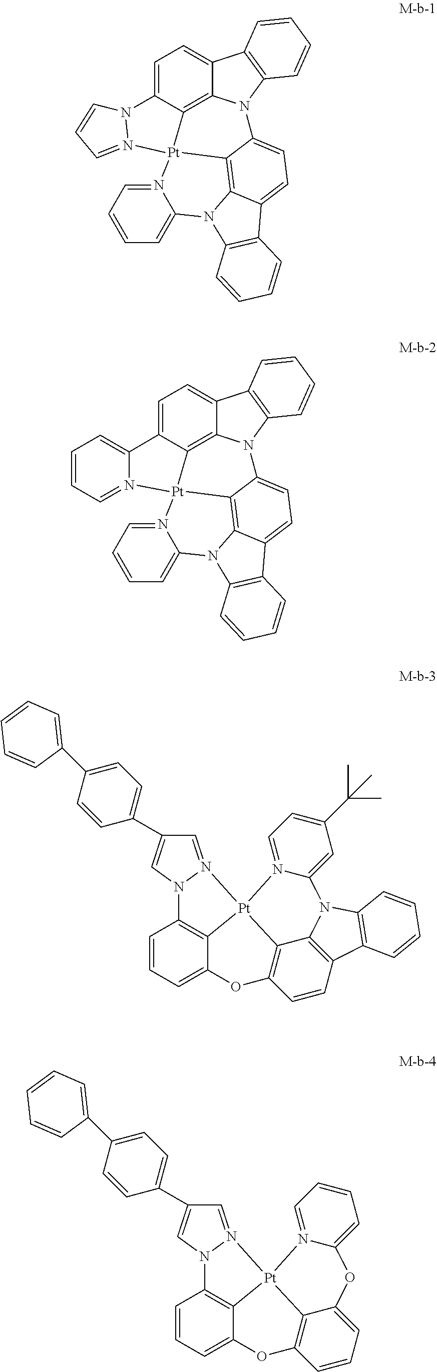

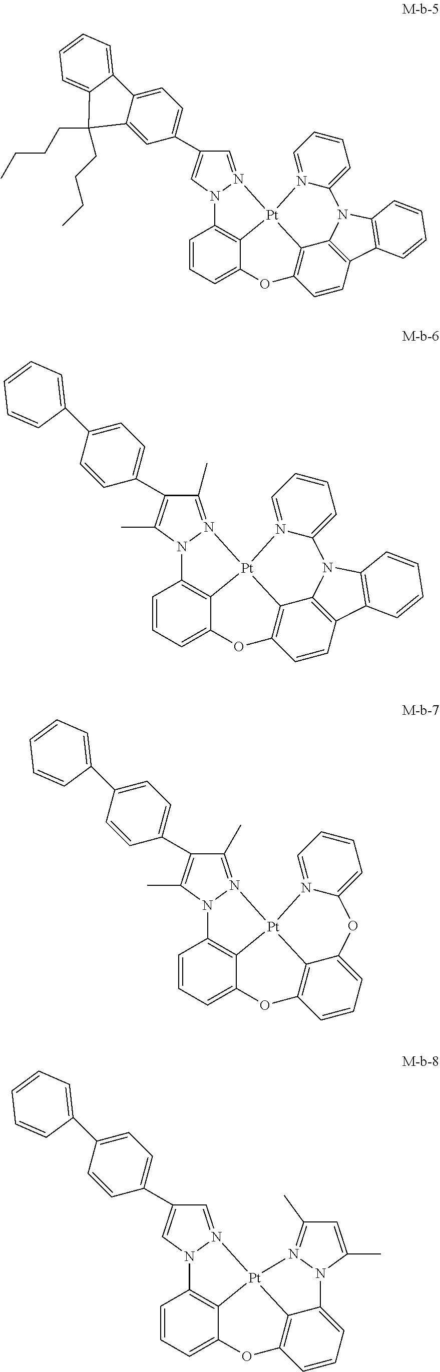

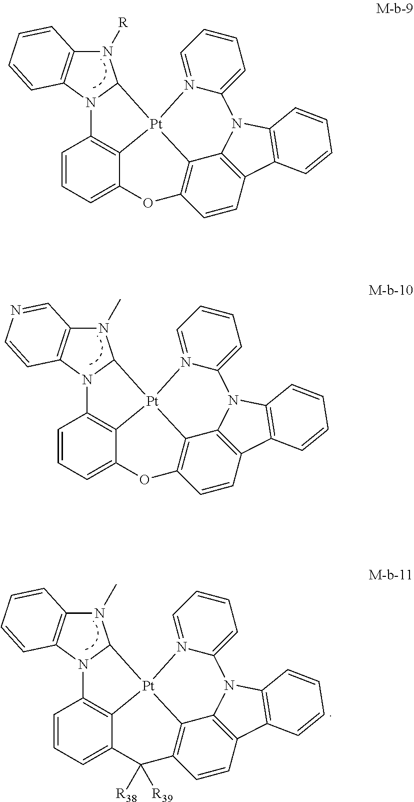

In the compounds, R, R₃₈, and R₃₉ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may further include a compound represented by any one selected from among Formula F-a to Formula F-c below. The compound represented by Formula F-a or Formula F-c below may be used as a fluorescence dopant material.

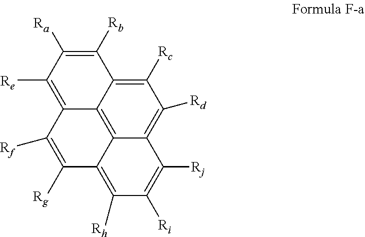

In Formula F-a above, two selected from among Rₐ to Rⱼ may each independently be substituted with *—NAr₁Ar₂. The others, which are not substituted with *—NAr₁Ar₂ among Rₐ to Rⱼ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *—NAr₁Ar₂Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. For example, at least one selected from Ar₁ and Ar₂ may be a heteroaryl group containing O or S as a ring-forming atom.

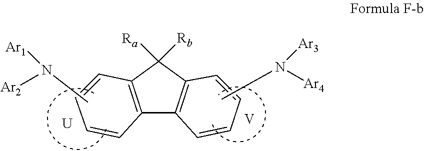

In Formula F-b above, Rₐ and R_{b} may be each independently a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-b above, Ar₁ to Ar₄ may be each independently a substituted or unsubstituted aryl group having 6 to 30 ring-forming carbon atoms, or a substituted or unsubstituted heteroaryl group having 2 to 30 ring-forming carbon atoms.

In Formula F-b, U and V may be each independently a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may be each independently 0 or 1. For example, in Formula F-b, it means that when the number of U or V is 1, one ring constitutes a fused ring at a portion indicated by U or V, and when the number of U or V is 0, a ring indicated by U or V does not exist. In some embodiments, when the number of U is 0 and the number of V is 1, or when the number of U is 1 and the number of V is 0, the fused ring having a fluorene core in Formula F-b may be a cyclic compound having four rings. In addition, when each number of U and V is 0, the fused ring in Formula F-b may be a cyclic compound having three rings. In addition, when each number of U and V is 1, the fused ring having a fluorene core in Formula F-b may be a cyclic compound having five rings.

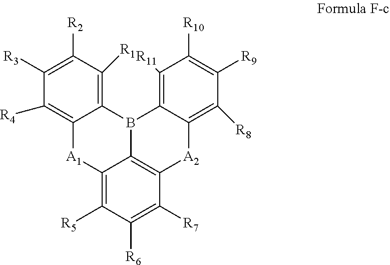

In Formula F-c, A₁ and A₂ may be each independently O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a fused ring. For example, when A₁ and A₂ are each independently NRₘ, A₁ may be bonded to R₄ or R₅ to form a ring. In addition, A₂ may be bonded to R₇ or R₈ to form a ring.

In an embodiment, the emission layer EML may include, any suitable dopant material generally used in the art. In some embodiments, the emission layer EML may include styryl derivatives (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and/or the derivatives thereof (e.g., 2,5,8,11-tetra-t-butylperylene (TBP)), pyrene and/or the derivatives thereof (e.g., 1,1-dipyrene, 1,4-dipyrenylbenzene, 1,4-bis(N,N-diphenylamino)pyrene), etc.

In an embodiment, when a plurality of emission layers EML are included, at least one emission layer EML may include any suitable phosphorescence dopant material generally used in the art. For example, a metal complex containing iridium (Ir), platinum (Pt), osmium (Os), aurum (Au), titanium (Ti), zirconium (Zr), hafnium (Hf), europium (Eu), terbium (Tb), and/or thulium (Tm) may be used as a phosphorescent dopant. In some embodiments, iridium(III) bis(4,6-difluorophenylpyridinato-N,C2′)picolinate (Firpic), bis(2,4-difluorophenylpyridinato)-tetrakis(1-pyrazolyl)borate iridium(III) (Fir6), and/or platinum octaethyl porphyrin (PtOEP) may be used as a phosphorescence dopant. However, embodiments of the present disclosure are not limited thereto.

In some embodiments, the emission layer EML in an embodiment may include a hole transport host and an electron transport host. and emission layer EML may include an auxiliary dopant and a light emitting dopant. A phosphorescent dopant material or a thermally delayed fluorescent dopant material may be included as the auxiliary dopant. For example, the emission layer EML in an embodiment may include the hole transport host, the electron transport host, the auxiliary dopant, and the light emitting dopant.

In addition, in the emission layer EML, an exciplex may be formed by the hole transport host and the electron transport host. In this case, a triplet energy of the exciplex formed by the hole transport host and the electron transport host may correspond to T1 that is a gap between a lowest unoccupied molecular orbital (LUMO) energy level of the electron transport host and a HOMO energy level of the hole transport host.

In an embodiment, the triplet energy (T1) of the exciplex formed by the hole transport host and the electron transport host may be about 2.4 eV to about 3.0 eV. In addition, the triplet energy of the exciplex may be a value smaller than an energy gap of each host material. The exciplex may, therefore, have a triplet energy of about 3.0 eV or less that is an energy gap between the hole transporting host and the electron transporting host.

In some embodiments, at least one emission layer EML may include a quantum dot material. The core of the quantum dot may be selected from a Group II-VI compound, a Group III-VI compound, a Group I-III-IV compound, a Group III-V compound, a Group III-II-V compound, a Group IV-VI compound, a Group IV element, a Group IV compound, or a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof.

The Group III-VI compound may include a binary compound such as In₂S₃ and/or In₂Se₃, a ternary compound such as InGaS₃ and/or InGaSe₃, or any combination thereof.

The Group I-III-VI compound may be selected from a ternary compound selected from the group consisting of AgInS, AgInS₂, CuInS, CuInS₂, AgGaS₂, CuGaS₂ CuGaO₂, AgGaO₂, AgAIO₂, and a mixture thereof, and/or a quaternary compound such as AgInGaS₂ and/or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. In some embodiments, the Group III-V compound may further include a Group II metal. For example, InZnP, etc., may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

In this case, the binary compound, the ternary compound, or the quaternary compound may be present in a particle at a uniform (e.g., substantially uniform) concentration distribution, or may be present in the same particle at a partially different concentration distribution. In addition, the quantum dot may have a core/shell structure in which one quantum dot surrounds another quantum dot. The core/shell structure may have a concentration gradient in which the concentration of elements present in the shell decreases along a direction toward the core.

In some embodiments, the quantum dot may have the above-described core/shell structure including a core containing nanocrystals and a shell surrounding the core. The shell of the quantum dot may serve as a protection layer to prevent or reduce the chemical deformation of the core to maintain semiconductor properties, and/or a charging layer to impart electrophoresis properties to the quantum dot. The shell may be a single layer or a multilayer. An example of the shell of the quantum dots may include a metal and/or non-metal oxide, a semiconductor compound, or a combination thereof.

For example, examples of the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and NiO, and/or a ternary compound such as MgAl₂O₄, CoFe₂O₄, NiFe₂O₄, and CoMn₂O₄, but embodiments of the present disclosure are not limited thereto.

In addition, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments of the present disclosure are not limited thereto.

The quantum dot may have a full width of half maximum (FWHM) of a light emitting wavelength spectrum of about 45 nm or less, about 40 nm or less, or, for example, about 30 nm or less, and color purity and/or color reproducibility may be improved in the above range. In addition, light emitted through such a quantum dot is emitted in all directions, and thus a wide viewing angle may be improved.

The form of the quantum dot is not particularly limited. For example, the quantum dot may have any suitable form generally used in the art. In some embodiments, a quantum dot in the form of spherical, pyramidal, multi-arm, and/or cubic nanoparticles, nanotubes, nanowires, nanofibers, nanoparticles, etc. may be used.

The quantum dot may control the color of emitted light according to the particle size thereof. Accordingly, the quantum dot may have various suitable light emission colors such as blue, red, and green.

In each of the light emitting elements ED of embodiments illustrated in FIGS. 3 to 6, the electron transport region ETR is provided on the emission layer EML. The electron transport region ETR may include at least one selected from of the hole blocking layer HBL, the electron transport layer ETL, and the electron injection layer EIL, but embodiments of the present disclosure are not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In addition, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL, an electron transport layer ETL/buffer layer/electron injection layer EIL are stacked in order from the emission layer EML, but embodiments of the present disclosure are not limited thereto. The electron transport region ETR may have a thickness, for example, from about 1,000 Å to about 1,500 Å.

The electron transport region ETR may be formed by using various suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1 below:

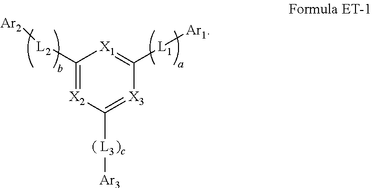

In Formula ET-1, at least one selected from among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may be each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may be each independently an integer of 0 to 10. In Formula ET-1, L₁ to L₃ may be each independently a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c are each independently an integer of 2 or more, L₁ to L₃ may be each independently a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, embodiments of the present disclosure are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), or a mixture thereof.

The electron transport region ETR may include at least one selected from among Compound ET1 to Compound ET36 below:

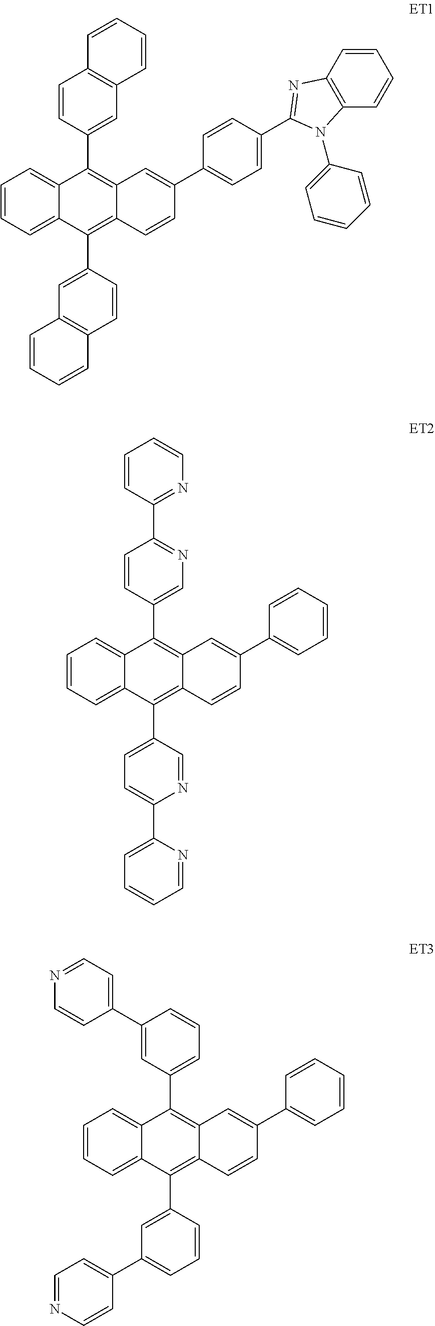 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

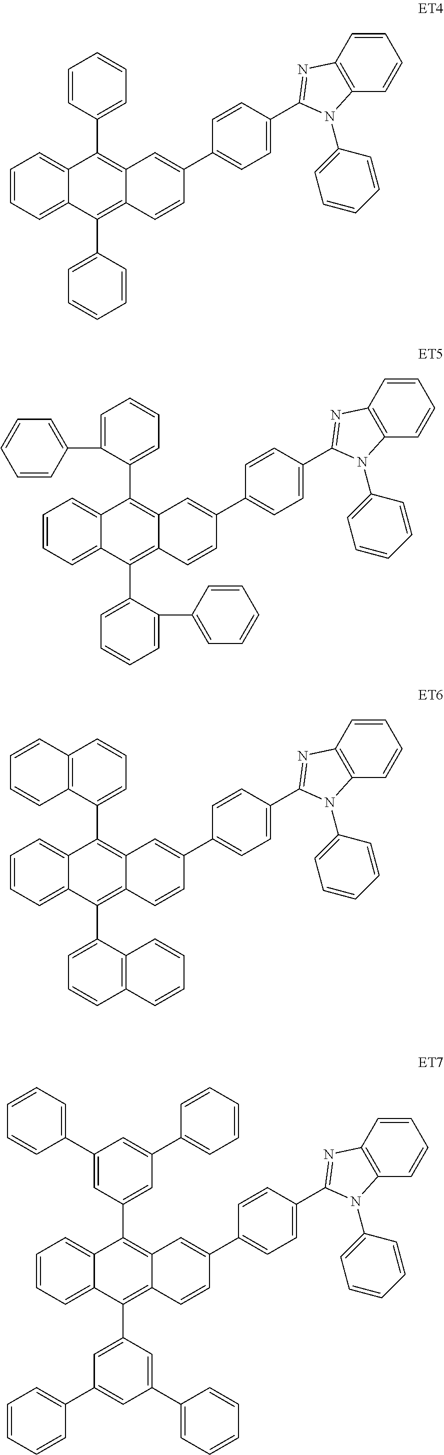 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

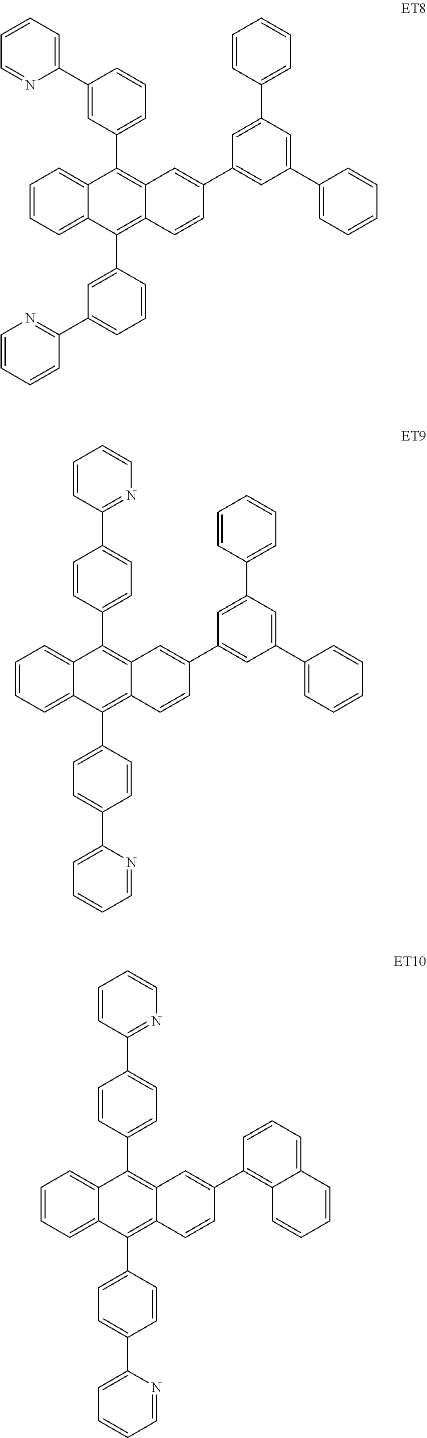 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

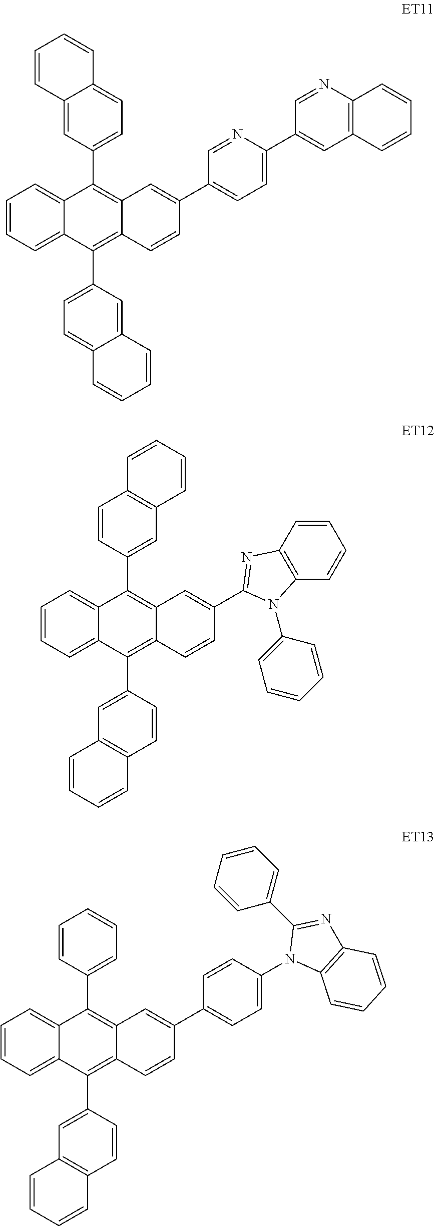 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

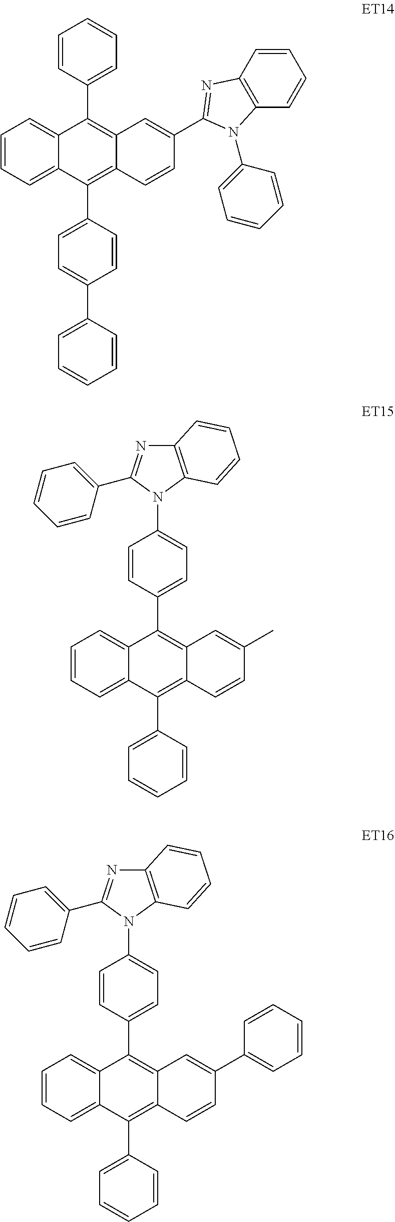 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

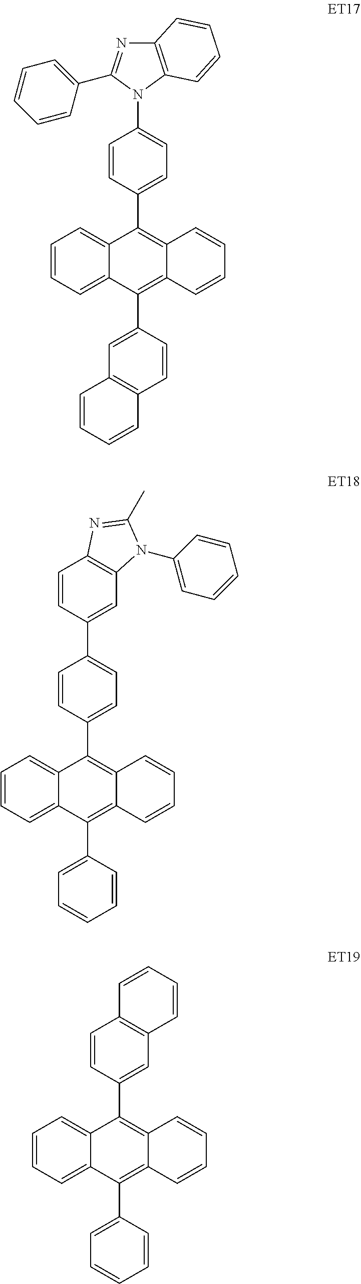

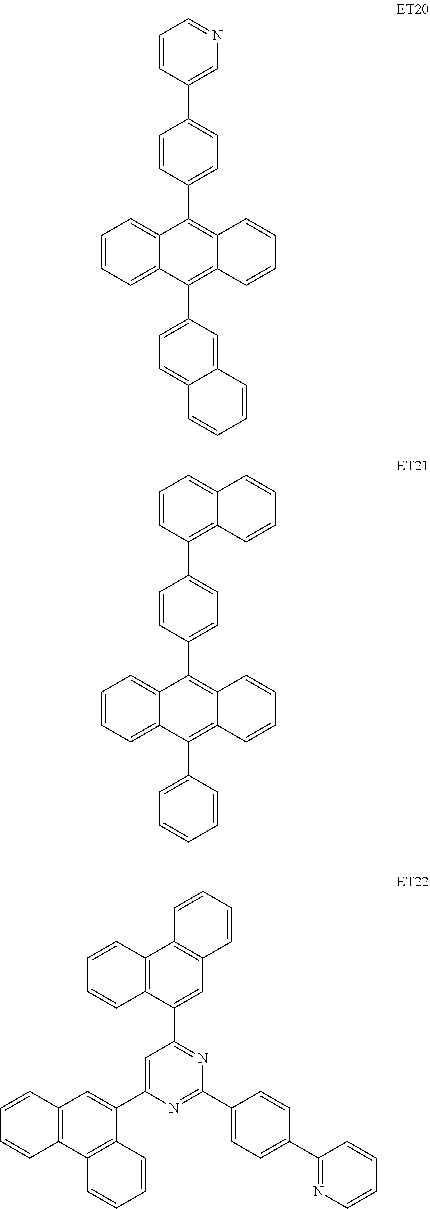

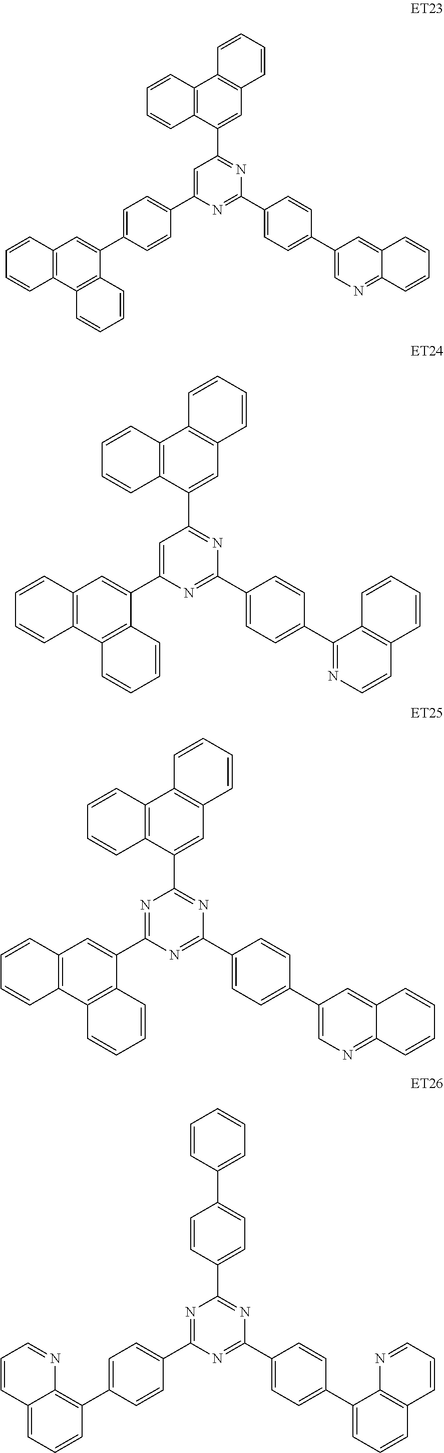 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

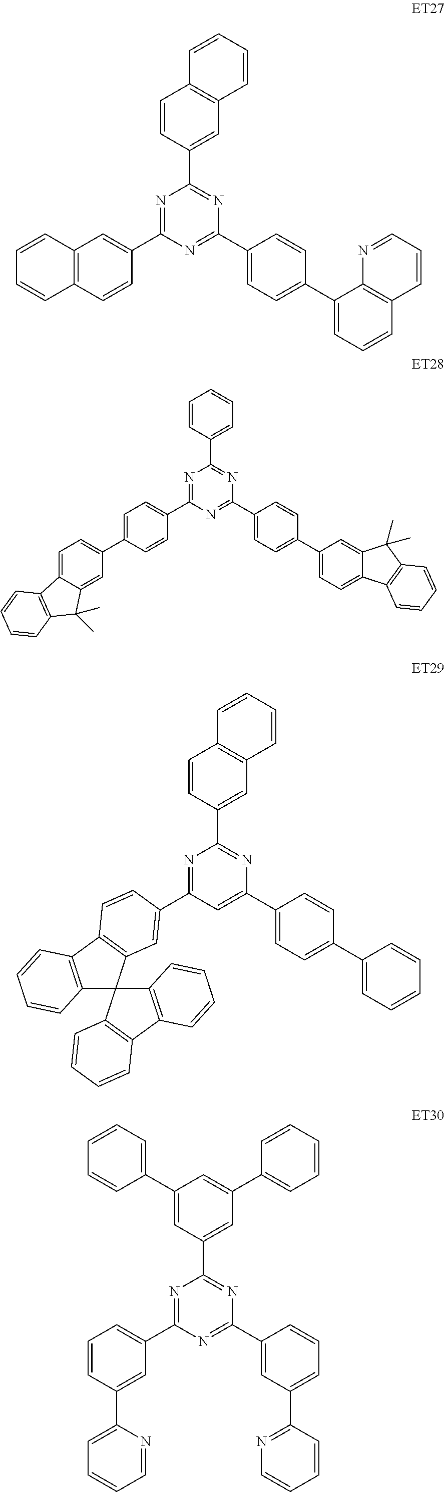

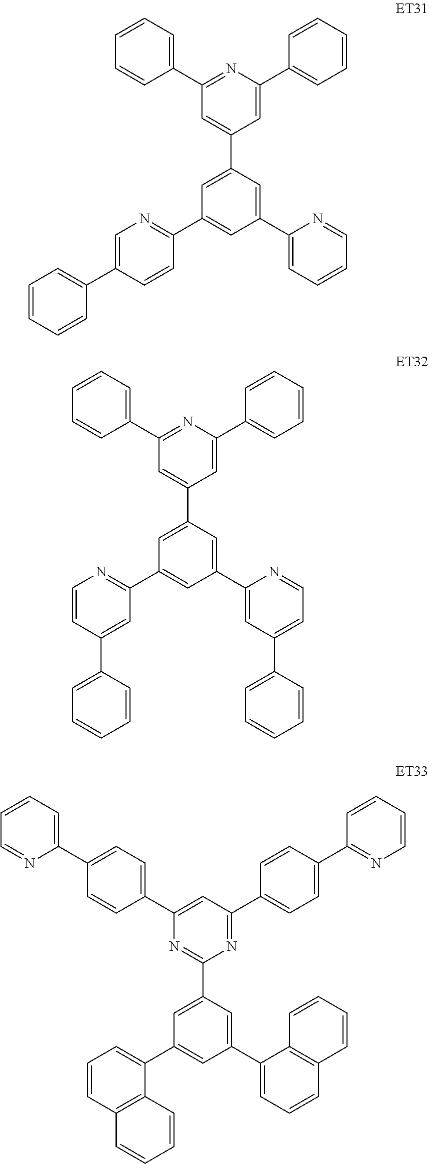 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

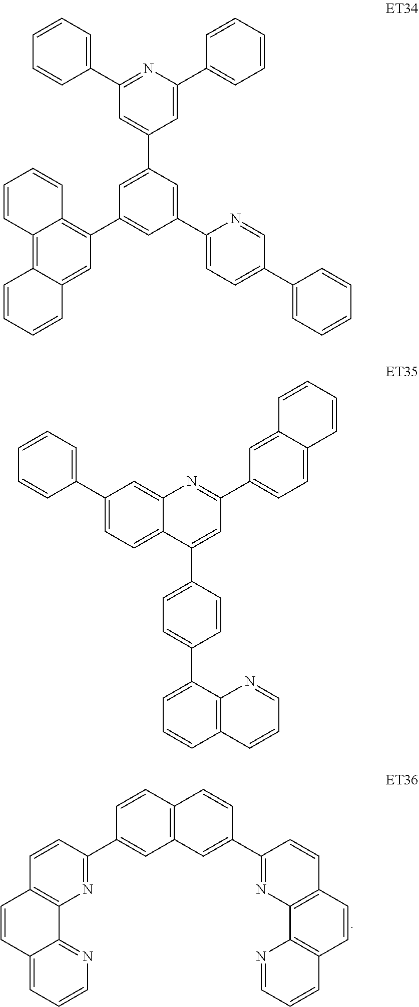

In addition, the electron transport regions ETR may include a metal halide such as LiF, NaCl, CsF, RbCl, RbI, CuI, and/or KI, a lanthanide metal such as Yb, and a co-deposited material of the metal halide and the lanthanide metal. For example, the electron transport region ETR may include KI:Yb, RbI:Yb, LiF:Yb, etc., as a co-deposited material. In some embodiments, the electron transport region ETR may be formed using a metal oxide such as Li₂O and/or BaO, and/or 8-hydroxyl-lithium quinolate (Liq), etc., but embodiments of the present disclosure are not limited thereto. The electron transport region ETR may also be formed of a mixture material of an electron transport material and an insulating organometallic salt. The organometallic salt may be a material having an energy band gap of about 4 eV or more. In some embodiments, the organometallic salt may include, for example, a metal acetate, a metal benzoate, a metal acetoacetate, a metal acetylacetonate, and/or a metal stearate.

The electron transport region ETR may further include at least one selected from 2,9-dimethyl-4,7-diphenyl-1,10-phenanthroline (BCP), diphenyl(4-(triphenylsilyl)phenyl)phosphine oxide (TSPO1), and 4,7-diphenyl-1,10-phenanthroline (Bphen) in addition to the above-described materials, but embodiments of the present disclosure are not limited thereto.

The electron transport region ETR may include the above-described compounds of the hole transport region in at least one selected from the electron injection layer EIL, the electron transport layer ETL, and the hole blocking layer HBL.

When the electron transport region ETR includes the electron transport layer ETL, the electron transport layer ETL may have a thickness of about 100 Å to about 1,000 Å, for example, about 150 Å to about 500 Å. If the thickness of the electron transport layer ETL satisfies the aforementioned range, suitable or satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the above-described range, suitable or satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but embodiments of the present disclosure are not limited thereto. For example, when the first electrode EL1 is an anode, the second electrode EL2 may be a cathode, and when the first electrode EL1 is a cathode, the second electrode EL2 may be an anode. The second electrode may include at least one selected from among Ag, Mg, Cu, Al, Pt, Pd, Au, Ni, Nd, Ir, Cr, L₁, Ca, LiF, Mo, Ti, W, In, Sn, and Zn, a compound of two or more selected from among these, a mixture of two or more selected from among these, and/or an oxide thereof.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, Yb, W, and/or a compound and/or mixture including these (e.g., AgMg, AgYb, or MgYb). In some embodiments, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, and/or the like.

In some embodiments, the second electrode EL2 may be connected with an auxiliary electrode. If the second electrode EL2 is connected with the auxiliary electrode, the resistance of the second electrode EL2 may be decreased.

In some embodiments, a capping layer CPL may further be on the second electrode EL2 of the light emitting element ED of an embodiment. The capping layer CPL may include a multilayer or a single layer.

In an embodiment, the capping layer CPL may be an organic layer or an inorganic layer. For example, when the capping layer CPL contains an inorganic material, the inorganic material may include an alkaline metal compound (e.g., LiF), an alkaline earth metal compound (e.g., MgF₂), SiON, SiNₓ, SiOy, etc.

For example, when the capping layer CPL includes an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol sol-9-yl)triphenylamine (TCTA), etc., and/or an epoxy resin, and/or acrylate such as methacrylate. However, embodiments of the present disclosure are not limited thereto, and the capping layer CPL may include at least one selected from among Compounds P1 to P5 below:

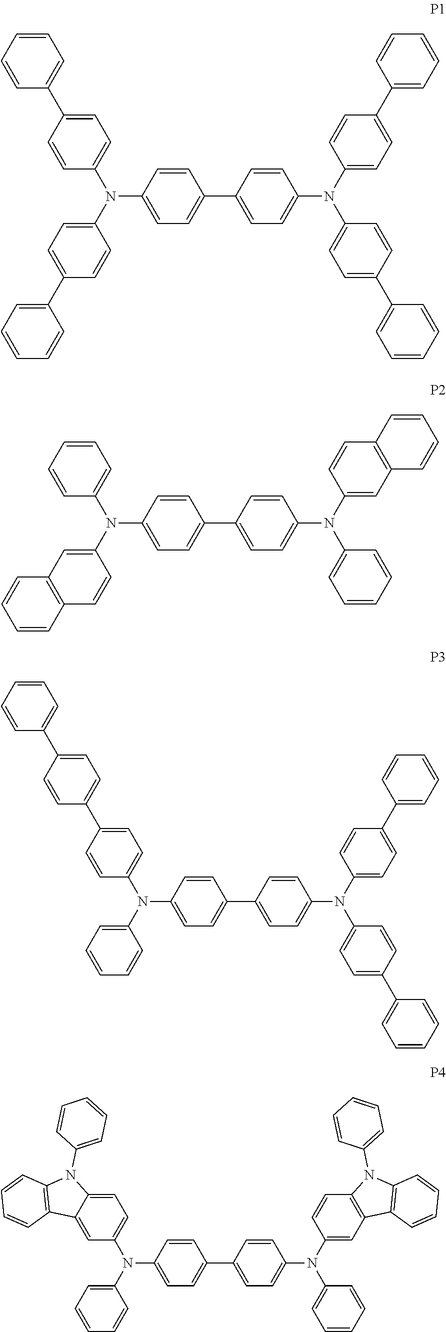 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

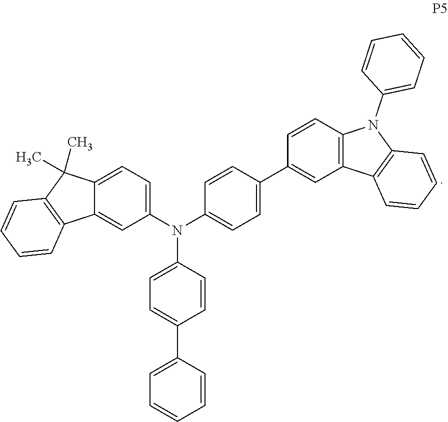 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be 1.6 or more with respect to light in a wavelength range of about 550 nm to about 660 nm.

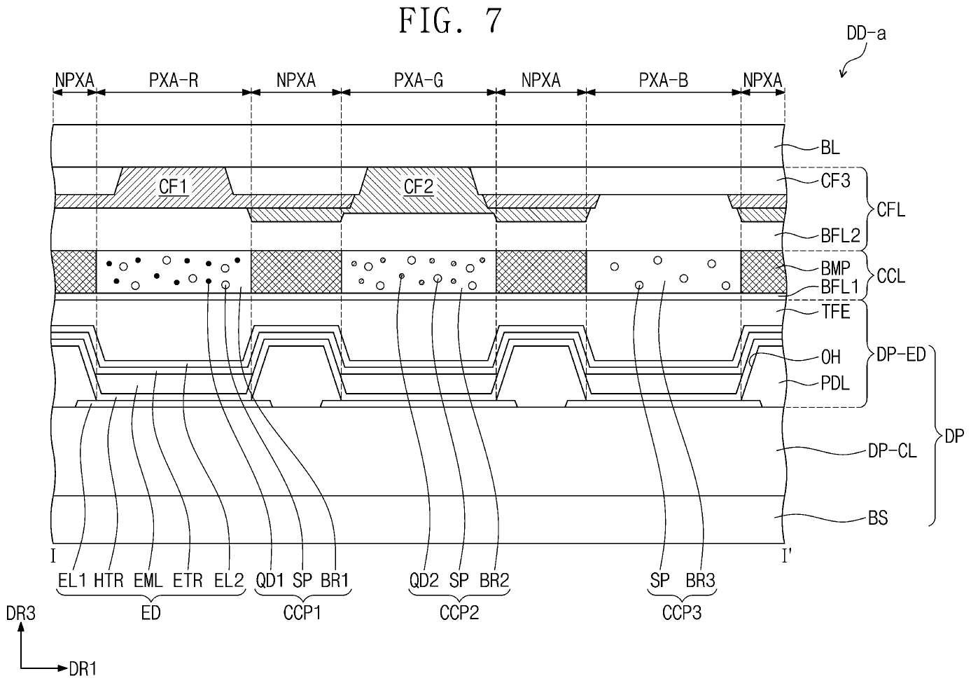

Each of FIGS. 7 to 10 is a cross-sectional view of a display device according to an embodiment of the inventive disclosure. Hereinafter, in describing the display devices of embodiments with reference to FIGS. 7 to 10, the duplicated features which have been described with respect to FIGS. 1 to 6 are not described again, but their differences will be mainly described.

Referring to FIG. 7, the display device DD-a according to an embodiment may include a display panel DP including a display element layer DP-ED, a light control layer CCL on the display panel DP, and a color filter layer CFL.

In an embodiment illustrated in FIG. 7, the display panel DP may include a base layer BS, a circuit layer DP-CL provided on the base layer BS, and the display element layer DP-ED, and the display element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, and a second electrode EL2 on the electron transport region ETR. The structures of the light emitting elements of FIGS. 3 to 6 as described above may be equally applied to the structure of the light emitting element ED illustrated in FIG. 7.

The hole transport region HTR of the light emitting element ED included in the display device DD-a according to an embodiment may include the above-described amine compound of an embodiment.

Referring to FIG. 7, the emission layer EML may be in an opening OH defined in a pixel defining film PDL. For example, the emission layer EML which is divided by the pixel defining film PDL and provided corresponding to each light emitting regions PXA-R, PXA-G, and PXA-B may emit light in the same wavelength range. In the display device DD of an embodiment, the emission layer EML may emit blue light. In some embodiments, unlike the configuration illustrated, in an embodiment, the emission layer EML may be provided as a common layer in the entire light emitting regions PXA-R, PXA-G, and PXA-B.

The light control layer CCL may be on the display panel DP. The light control layer CCL may include a light conversion body. The light conversion body may be a quantum dot, a phosphor, and/or the like. The light conversion body may emit provided light by converting the wavelength thereof. In some embodiments, the light control layer CCL may a layer containing the quantum dot and/or a layer containing the phosphor.

The light control layer CCL may include a plurality of light control parts CCP1, CCP2 and CCP3. The light control parts CCP1, CCP2, and CCP3 may be spaced apart from each other.

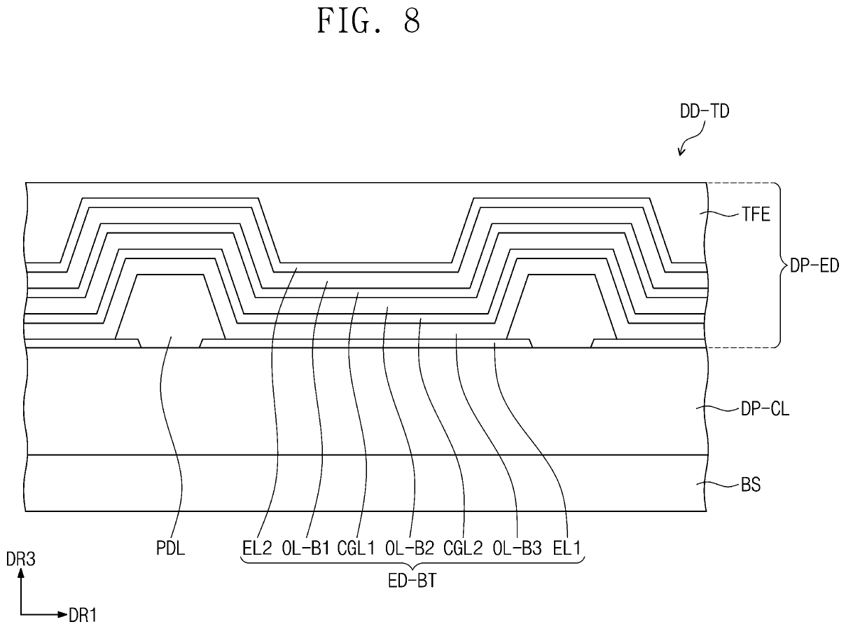

Referring to FIG. 7, divided patterns BMP may be between the light control parts CCP1, CCP2 and CCP3 which are spaced apart from each other, but embodiments of the present disclosure are not limited thereto. FIG. 8 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts a first color light provided from the light emitting element ED into a second color light, a second light control part CCP2 containing a second quantum dot QD2 which converts the first color light into a third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same as described above may be applied with respect to the quantum dots QD1 and QD2.

In addition, the light control layer CCL may further include a scatterer SP (e.g., a light scatterer SP). The first light control part CCP1 may include the first quantum dot QD1 and the scatterer SP, the second light control part CCP2 may include the second quantum dot QD2 and the scatterer SP, and the third light control part CCP3 may not include any quantum dot but include the scatterer SP.

The scatterer SP may be inorganic particles. For example, the scatterer SP may include at least one selected from TiO₂, ZnO, Al₂O₃, SiO₂, and hollow sphere silica. The scatterer SP may include any one selected from among TiO₂, ZnO, Al₂O₃, SiO₂, and hollow sphere silica, and/or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 are media in which the quantum dots QD1 and QD2 and the scatterer SP are dispersed, and may be formed of various suitable resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may be on the light control parts CCP1, CCP2, and CCP3 to block or reduce exposure of the light control parts CCP1, CCP2 and CCP3 to moisture/oxygen. In some embodiments, the barrier layer BFL1 may cover the light control parts CCP1, CCP2, and CCP3. In addition, a barrier layer BFL2 may be provided between the light control parts CCP1, CCP2, and CCP3 and filters CF1, CF2, and CF3.

The barrier layers BFL1 and BFL2 may include at least one inorganic layer. In some embodiments, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In some embodiments, the barrier layers BFL1 and BFL2 may further include an organic film. The barrier layers BFL1 and BFL2 may be formed of a single layer or a plurality of layers.

In the display device DD of an embodiment, the color filter layer CFL may be on the light control layer CCL. For example, the color filter layer CFL may be directly on the light control layer CCL. In this case, the barrier layer BFL2 may be omitted.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment and/or dye. The first filter CF1 may include a red pigment and/or dye, the second filter CF2 may include a green pigment and/or dye, and the third filter CF3 may include a blue pigment and/or dye. Embodiments of the present disclosure are not limited thereto, however, and the third filter CF3 may not include a pigment or dye. The third filter CF3 may include a polymeric photosensitive resin and may not include a pigment or dye. The third filter CF3 may be transparent. The third filter CF3 may be formed of a transparent photosensitive resin.

Furthermore, in an embodiment, the first filter CF1 and the second filter CF2 may be a yellow filter. The first filter CF1 and the second filter CF2 may not be separated but be provided as one filter. The first to third filters CF1, CF2, and CF3 may be corresponding to the red light emitting region PXA-R, the green light emitting region PXA-G, and the blue light emitting region PXA-B, respectively.

In some embodiments, the color filter layer CFL may include a light shielding part. The color filter layer CFL may include a light shielding part that overlaps at the boundaries of neighboring filters CF1, CF2, and CF3. The light shielding part may be a black matrix. The light shielding part may include an organic light shielding material and/or an inorganic light shielding material containing a black pigment and/or dye. The light shielding part may separate boundaries between the adjacent filters CF1, CF2, and CF3. In addition, in an embodiment, the light shielding part may be formed of a blue filter.

A base substrate BL may be on the color filter layer CFL. The base substrate BL may be a member which provides a base surface that the color filter layer CFL, the light control layer CCL, and the like are on. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may be omitted.

FIG. 8 is a cross-sectional view illustrating a portion of a display device according to an embodiment. In the display device DD-TD of an embodiment, the light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR with the emission layer EML (FIG. 7) therebetween.

In some embodiments, the light emitting element ED-BT included in the display device DD-TD of an embodiment may be a light emitting element having a tandem structure and including a plurality of emission layers.

In an embodiment illustrated in FIG. 8, all light beams respectively emitted from the light emitting structures OL-B1, OL-B2, and OL-B3 may be blue light. However, embodiments of the present disclosure are not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may emit white light.

Charge generation layers CGL1 and CGL2 may be respectively between two of the neighboring light emitting structures OL-B1, OL-B2, and OL-B3. The charge generation layers CGL1 and CGL2 may include a p-type charge generation layer and/or an n-type charge generation layer.

At least one selected from the light emitting structures OL-B1, OL-B2, and OL-B3 included in the display device DD-TD of an embodiment may include the above-described amine compound of an embodiment.

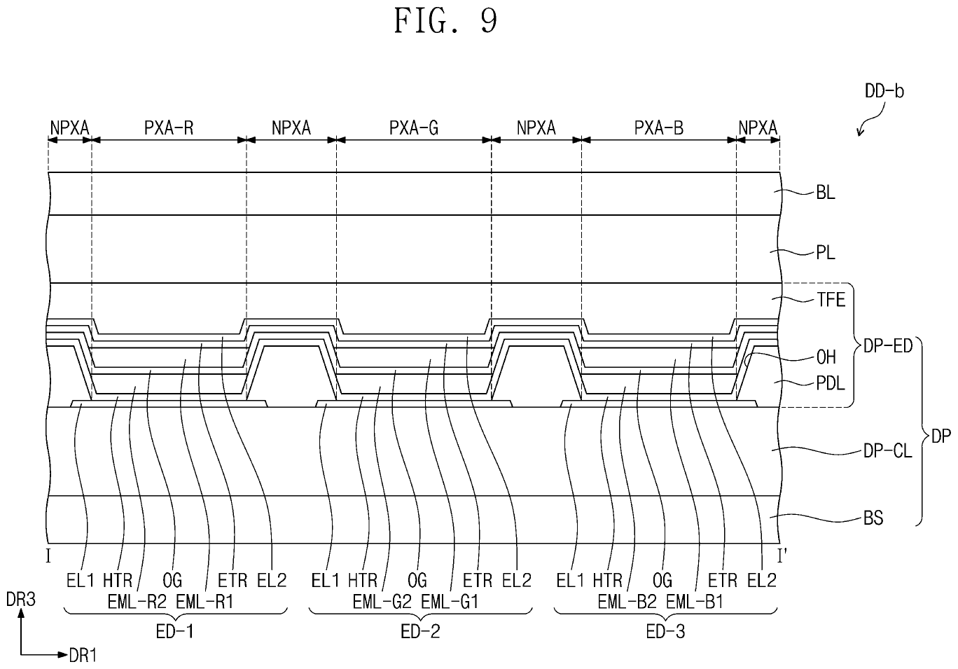

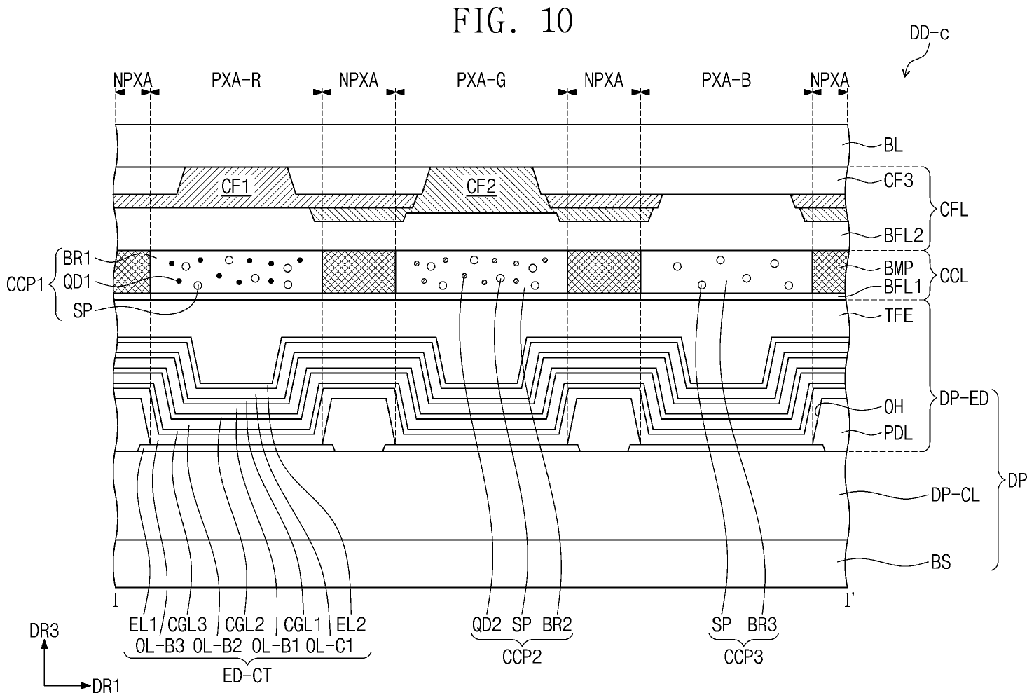

Referring to FIG. 9, the display device DD-b according to an embodiment may include light emitting elements ED-1, ED-2, and ED-3 in which two emission layers are stacked. Compared with the display device DD of an embodiment illustrated in FIG. 2, an embodiment illustrated in FIG. 9 has a difference in that the first to third light emitting elements ED-1, ED-2, and ED-3 each include two emission layers stacked in the thickness direction. In each of the first to third light emitting elements ED-1, ED-2, and ED-3, the two emission layers may emit light in the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. An emission auxiliary part OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. For example, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, embodiments of the present disclosure are not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be between the hole transport region HTR and the emission auxiliary part OG. The second red emission layer EML-R2, the second green emission layer EML-G2, and the second blue emission layer EML-B2 may be between the emission auxiliary part OG and the electron transport region ETR.

In some embodiments, the first light emitting element ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked. The second light emitting element ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting element ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

In some embodiments, an optical auxiliary layer PL may be on the display element layer DP-ED. The optical auxiliary layer PL may include a polarizing layer. The optical auxiliary layer PL may be on the display panel DP and control reflected light in the display panel DP due to external light. In some embodiments, the optical auxiliary layer PL may be omitted.

Unlike FIGS. 8 and 9, FIG. 10 illustrates that a display device DD-c includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be between the first to fourth light emitting structures OL-B1, OL-B2, OL-B3, and OL-C1. Among the four light emitting structures, the first to third light emitting structures OL-B1, OL-B2, and OL-B3 may emit blue light, and the fourth light emitting structure OL-C1 may emit green light. However, embodiments of the present disclosure are not limited thereto, and the first to fourth light emitting structures OL-B1, OL-B2, OL-B3, and OL-C1 may emit light beams in different wavelength regions.

The charge generation layers CGL1, CGL2, and CGL3 between adjacent light emitting structures OL-B1, OL-B2, OL-B3, and OL-C1 may include a p-type charge generation layer and/or an n-type charge generation layer.

At least one selected from among the light emitting structures OL-B1, OL-B2, OL-B3, and OL-C1 included in the display device DD-c of an embodiment may include the above-described amine compound of an embodiment.

The light emitting element ED according to an embodiment of the inventive disclosure may include the above-described amine compound of an embodiment in at least one functional layer between the first electrode EL1 and the second electrode EL2, thereby exhibiting improved luminous efficiency and service life characteristics. The light emitting element ED according to an embodiment may include the above-described amine compound of an embodiment in at least one selected from of the hole transport region HTR, the emission layer EML, and the electron transport region ETR between the first electrode EL1 and the second electrode EL2, or in a capping layer CPL. For example, the amine compound according to an embodiment may be included in the hole transport region HTR of the light emitting element ED of an embodiment, and the light emitting element of an embodiment may exhibit high efficiency and long service life characteristics.

The amine compound of an embodiment as described above includes the first core, and the second and third substituents, and thus the stability of material may be increased and the hole transport property may be improved. Accordingly, the light emitting element including the amine compound of an embodiment may have improved service life and efficiency of the light emitting element. In addition, the light emitting element of an embodiment includes the amine compound according to an embodiment in the hole transport layer, thereby exhibiting increased efficiency and service life characteristics.

Hereinafter, with reference to Examples and Comparative Examples, an amine compound according to an embodiment of the inventive disclosure and a light emitting element of an embodiment of the inventive disclosure will be described in more detail. In addition, Examples described below are only illustrations to assist the understanding of the subject matter of the inventive disclosure, and the scope of the inventive disclosure is not limited thereto.

### EXAMPLES

### 1. Synthesis of Amine Compound

First, a synthetic method of an amine compound according to the current embodiment will be described in more detail by illustrating synthetic methods of Compounds A10, D2, I7, AC15, AM3, and AX12. Also, in the following descriptions, the synthetic method of the amine compound is provided as an example, but the synthetic method according to an embodiment of the present disclosure is not limited to Examples below. In the synthesis of amine compounds, the molecular weights of the synthesized compounds were obtained by measuring FAB-MS using JMS-700V made by JEOL, Ltd.

### (1) Synthesis of Compound A10

Amine Compound A10 according to an example may be synthesized by, for example, the reaction below.

### Synthesis of Compound IM-1

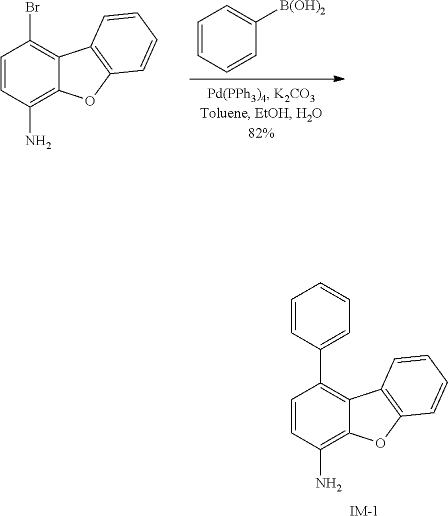

In an Ar atmosphere, in a 2,000 mL three-neck flask, 1-bromodibenzo[b,d]furan-4-amine (25.00 g, 95.38 mmol), phenylboronic acid (12.79 g, 1.1 equiv, 104.9 mmol), Pd(PPh₃)₄ (11.0 g, 0.10 equiv, 9.54 mmol), K₂CO₃ (26.36 g, 2.0 equiv, 190.8 mmol), toluene (380 mL), EtOH (190 mL), and H₂O (100 mL) were sequentially added, and then heated and stirred under reflux. After the reaction solution was air-cooled to room temperature, organic layers were separated and obtained by adding water and toluene to the reaction solvent. The organic layers were washed with saline, and then dried over MgSO₄. MgSO₄ was filtered and the organic layers were concentrated to obtain a crude product. The obtained crude product was purified to obtain Compound IM-1 (20.24 g, yield 82%).

### Synthesis of Compound IM-2

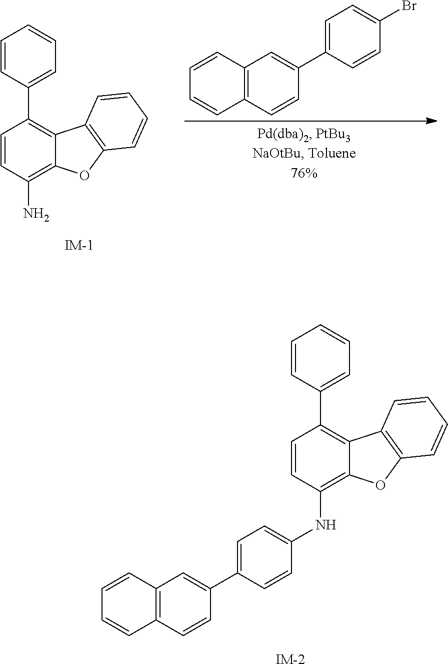

In an Ar atmosphere, in a 500 mL three-neck flask, Compound IM-1 (6.00 g, 23.1 mmol), Pd(dba)₂ (0.67 g, 0.05 equiv, 1.2 mmol), NaO^{t}Bu (2.22 g, 1.0 equiv, 23.1 mmol), toluene (230 mL), 2-(4-bromophenyl)naphthalene (6.55 g, 1.0 equiv, 23.1 mmol), and P^{t}Bu₃ (0.94 g, 0.2 equiv, 4.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and were then washed with saline and dried over MgSO₄. MgSO₄ was filtered and the organic layers were concentrated to obtain a crude product. The obtained crude product was purified to obtain Compound IM-2 (8.10 g, yield 76%).

By measuring FAB-MS, a mass number of/z=461 was observed by molecular ion peak, thereby identifying Compound IM-2.

### Synthesis of Compound A10

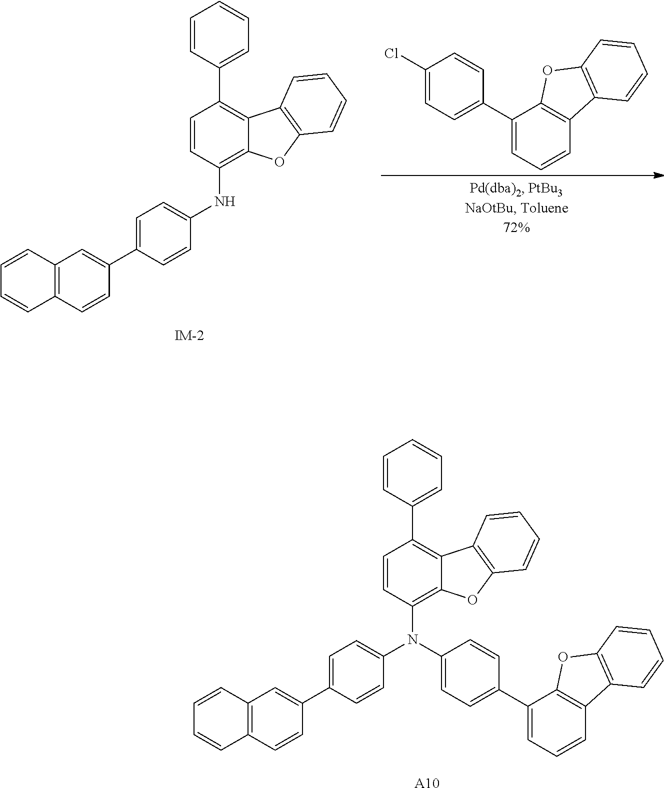

In an Ar atmosphere, in a 1,000 mL three-neck flask, Compound IM-2 (8.10 g, 17.6 mmol), Pd(dba)₂ (0.50 g, 0.05 equiv, 0.88 mmol), NaO^{t}Bu (1.69 g, 1.0 equiv, 17.6 mmol), toluene (180 mL), 4-(4-chlorophenyl)dibenzo[b,d]furan (4.89 g, 1.0 equiv, 17.6 mmol), and P^{t}Bu₃ (0.71 g, 0.2 equiv, 3.5 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and were then washed with saline and dried over MgSO₄. MgSO₄ was filtered and the organic layers were concentrated to obtain a crude product. The obtained crude product was purified to obtain Compound A-10 (8.90 g, yield 72%).

By measuring FAB-MS, a mass number of/z=703 was observed by molecular ion peak, thereby identifying Compound A-10.

### (2) Synthesis of Compound D2

Amine Compound D2 according to an example may be synthesized by, for example, the reaction below.

### Synthesis of Compound IM-3

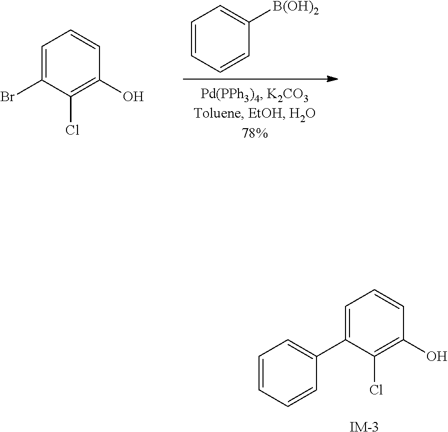

In an Ar atmosphere, in a 2,000 mL three-neck flask, 3-bromo-2-chlorophenol (30.00 g, 144.6 mmol), phenylboronic acid (19.40 g, 1.1 equiv, 159.1 mmol), Pd(PPh₃)₄ (16.71 g, 0.10 equiv, 14.46 mmol), K₂CO₃ (39.97 g, 2.0 equiv, 289.2 mmol), toluene (580 mL), EtOH (280 mL), and H₂O (140 mL) were sequentially added, and then heated and stirred under reflux. After the reaction solution was air-cooled to room temperature, organic layers were separated and obtained by adding water and toluene to the reaction solvent. The organic layers were washed with saline, and then dried over MgSO₄. MgSO₄ was filtered and the organic layers were concentrated to obtain a crude product. The obtained crude product was purified to obtain Compound IM-3 (23.11 g, yield 78%).

By measuring FAB-MS, a mass number of/z=204 was observed by molecular ion peak, thereby identifying Compound IM-3.

### Synthesis of Compound IM-4

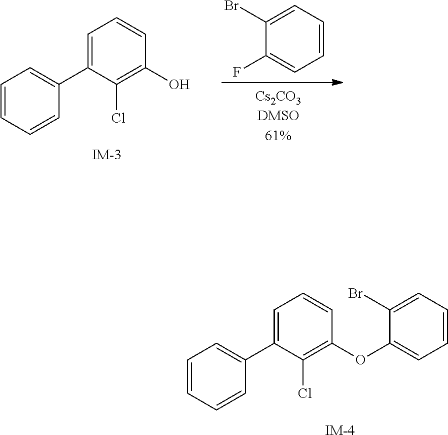

In an Ar atmosphere, in a 300 mL three-neck flask, Compound IM-3 (23.11 g, 112.9 mmol), Cs₂CO₃ (73.59 g, 2.0 equiv, 225.9 mmol), DMSO (110 mL), and 1-bromo-2-fluorobenzene (39.52 g, 2.0 equiv, 225.9 mmol) were sequentially added, and then heated and stirred at about 120° C. After the reaction solution was air-cooled to room temperature, organic layers were separated and obtained by adding water and toluene to the reaction solvent. The organic layers were washed with saline, and then dried over MgSO₄. MgSO₄ was filtered and the organic layers were concentrated to obtain a crude product. The obtained crude product was purified to obtain Compound IM-4 (24.74 g, yield 61%).

By measuring FAB-MS, a mass number of m/z=358 was observed by molecular ion peak, thereby identifying Compound IM-4.

### Synthesis of Compound IM-5

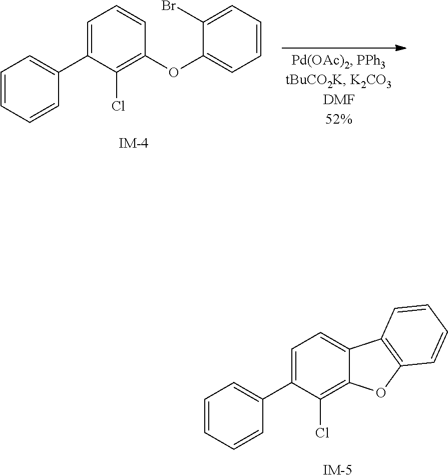

In an Ar atmosphere, in a 1,000 mL three-neck flask, Compound IM-4 (24.74 g, 68.79 mmol), tBuCO₂K (9.65 g, 1.0 equiv, 68.8 mmol), Pd(OAc)₂ (3.09 g, 0.2 equiv, 13.8 mmol), PPh₃ (3.61 g, 0.2 equiv, 13.8 mmol), K₂CO₃ (28.52 g, 3.0 equiv, 206.4 mmol), and DMSO (340 mL) were sequentially added, and then heated and stirred at about 120° C. After the reaction solution was air-cooled to room temperature, organic layers were separated and obtained by adding water and toluene to the reaction solvent. The organic layers were washed with saline, and then dried over MgSO₄. MgSO₄ was filtered and the organic layers were concentrated to obtain a crude product. The obtained crude product was purified to obtain Compound IM-5 (9.95 g, yield 52%).

By measuring FAB-MS, a mass number of m/z=278 was observed by molecular ion peak, thereby identifying Compound IM-5.

### Synthesis of Compound IM-6

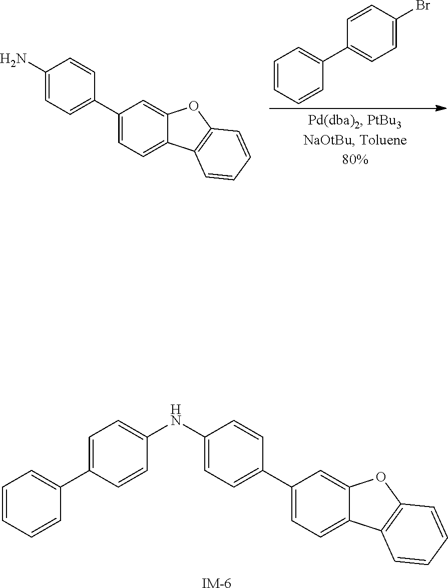

In an Ar atmosphere, in a 2,000 mL three-neck flask, 4-(dibenzo[b,d]furan-3-yl)aniline (15.00 g, 57.85 mmol), Pd(dba)₂ (1.66 g, 0.05 equiv, 2.89 mmol), NaO^{t}Bu (5.56 g, 1.0 equiv, 57.9 mmol), toluene (580 mL), 4-bromo-1,1′-biphenyl (13.48 g, 1.0 equiv, 57.85 mmol), and P^{t}Bu₃ (2.34 g, 0.2 equiv, 11.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and were washed with saline and then dried over MgSO₄. MgSO₄ was filtered and the organic layers were concentrated to obtain a crude product. The obtained crude product was purified to obtain Compound IM-6 (19.04 g, yield 80%).

By measuring FAB-MS, a mass number of m/z=411 was observed by molecular ion peak, thereby identifying Compound IM-6.

### Synthesis of Compound D2

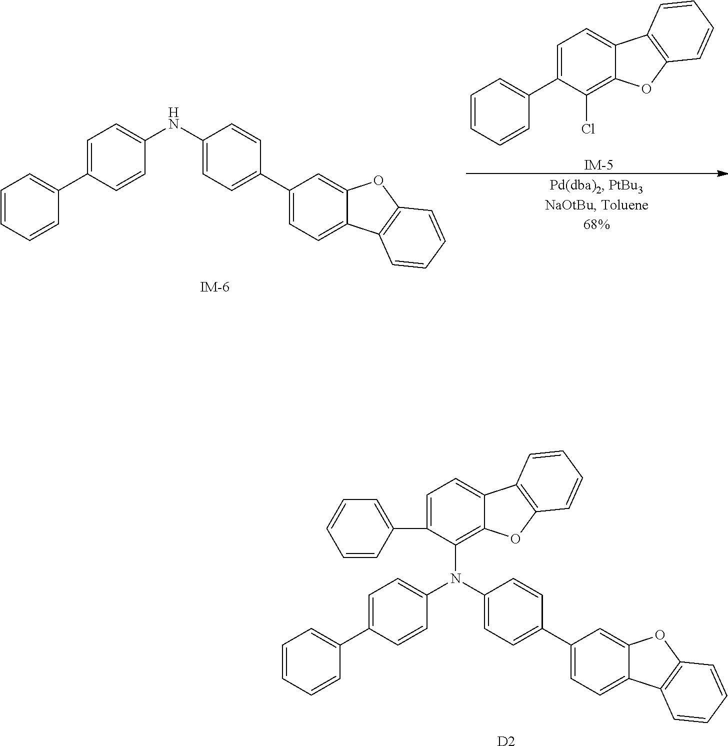

In an Ar atmosphere, in a 500 mL three-neck flask, Compound IM-6 (7.38 g, 17.9 mmol), Pd(dba)₂ (0.52 g, 0.05 equiv, 0.90 mmol), NaO^{t}Bu (1.72 g, 1.0 equiv, 17.9 mmol), toluene (180 mL), Compound IM-5 (5.00 g, 1.0 equiv, 17.9 mmol), and P^{t}Bu₃ (0.73 g, 0.2 equiv, 3.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then were washed with saline and dried over MgSO₄. MgSO₄ was filtered and the organic layers were concentrated to obtain a crude product. The obtained crude product was purified to obtain Compound D2 (8.00 g, yield 68%).

By measuring FAB-MS, a mass number of m/z=653 was observed by molecular ion peak, thereby identifying Compound D2.

### (3) Synthesis of Compound 17

Amine Compound 17 according to an example may be synthesized by, for example, the reaction below.

### Synthesis of Compound IM-7

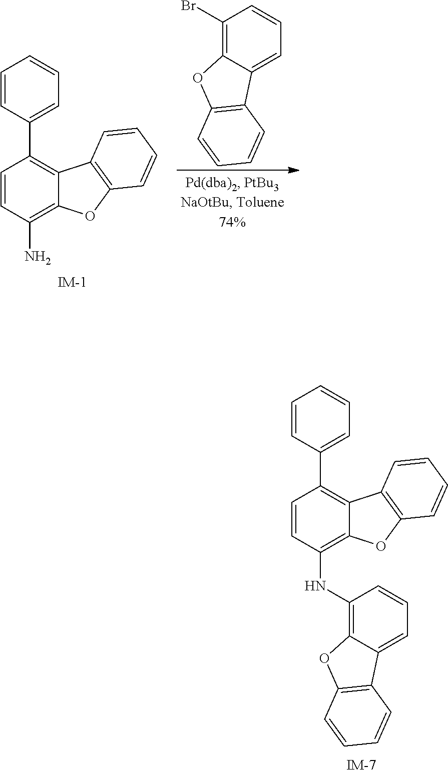

In an Ar atmosphere, in a 500 mL three-neck flask, Compound IM-1 (5.00 g, 19.3 mmol), Pd(dba)₂ (0.55 g, 0.05 equiv, 0.96 mmol), NaO^{t}Bu (1.85 g, 1.0 equiv, 19.3 mmol), toluene (190 mL), 4-bromodibenzo[b,d]furan (4.76 g, 1.0 equiv, 19.3 mmol), and P^{t}Bu₃ (0.78 g, 0.2 equiv, 3.86 mmol) were sequentially added, and then heated and stirred at about 80° C. After the reaction solution was air-cooled to room temperature, organic layers were separated and obtained by adding water to the reaction solvent. The organic layers were further extracted by adding toluene to a water layer, and then were washed with saline and dried over MgSO₄. MgSO₄ was filtered and the organic layers were concentrated to obtain a crude product. The obtained crude product was purified to obtain Compound IM-7 (6.08 g, yield 74%).

By measuring FAB-MS, a mass number of m/z=425 was observed by molecular ion peak, thereby identifying Compound IM-7.

### Synthesis of Compound 17

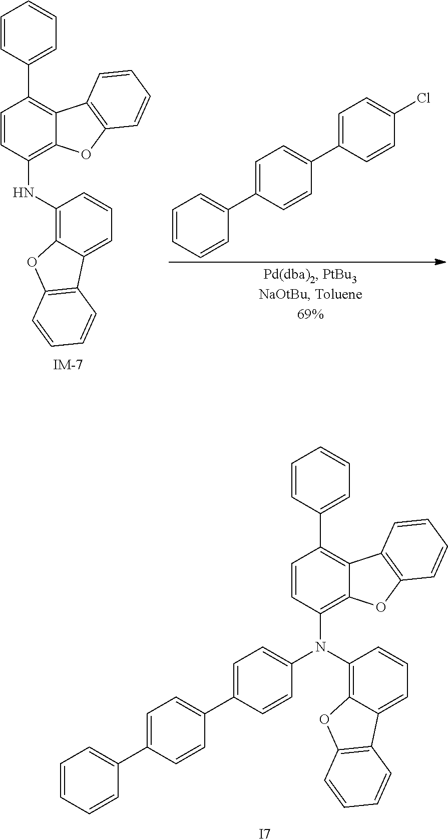

In an Ar atmosphere, in a 500 mL three-neck flask, Compound IM-7 (6.08 g, 14.3 mmol), Pd(dba)₂ (0.41 g, 0.05 equiv, 0.71 mmol), NaO^{t}Bu (1.37 g, 1.0 equiv, 14.3 mmol), toluene (140 mL), 4-chloro-1,1′:4′,1″-terphenyl (3.78 g, 1.0 equiv, 14.3 mmol), and P^{t}Bu₃ (0.58 g, 0.2 equiv, 2.86 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then were washed with saline and dried over MgSO₄. MgSO₄ was filtered and the organic layers were concentrated to obtain a crude product. The obtained crude product was purified to obtain Compound 17 (6.45 g, yield 69%).

By measuring FAB-MS, a mass number of/z=653 was observed by molecular ion peak, thereby identifying Compound 17.

### (4) Synthesis of Compound AC15

Amine Compound AC15 according to an example may be synthesized by, for example, the reaction below.

### Synthesis of Compound IM-8

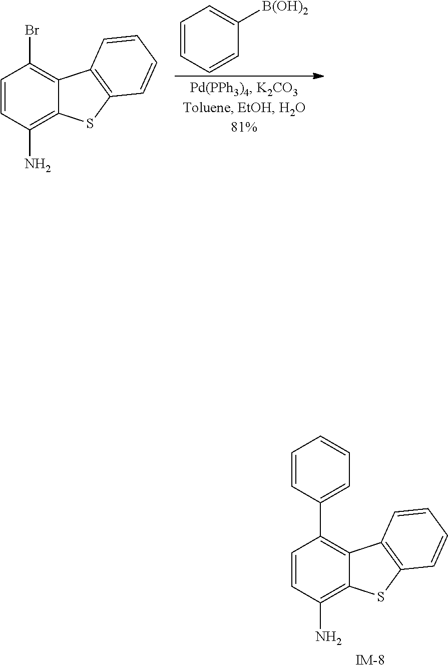

In an Ar atmosphere, in a 1,000 mL three-neck flask, 1-bromodibenzo[b,d]thiophen-4-amine (20.00 g, 72.21 mmol), phenylboronic acid (9.68 g, 1.1 equiv, 79.4 mmol), Pd(PPh₃)₄ (8.34 g, 0.10 equiv, 7.22 mmol), K₂CO₃ (19.96 g, 2.0 equiv, 144.4 mmol), toluene (280 mL), EtOH (140 mL), and H₂O (70 mL) were sequentially added, and then heated and stirred under reflux. After the reaction solution was air-cooled to room temperature, organic layers were separated and obtained by adding water and toluene to the reaction solvent. The organic layers were washed with saline, and then dried over MgSO₄. MgSO₄ was filtered and the organic layers were concentrated to obtain a crude product. The obtained crude product was purified to obtain Compound IM-8 (16.07 g, yield 81%).

By measuring FAB-MS, a mass number of/z=275 was observed by molecular ion peak, thereby identifying Compound IM-8.

### Synthesis of Compound IM-9

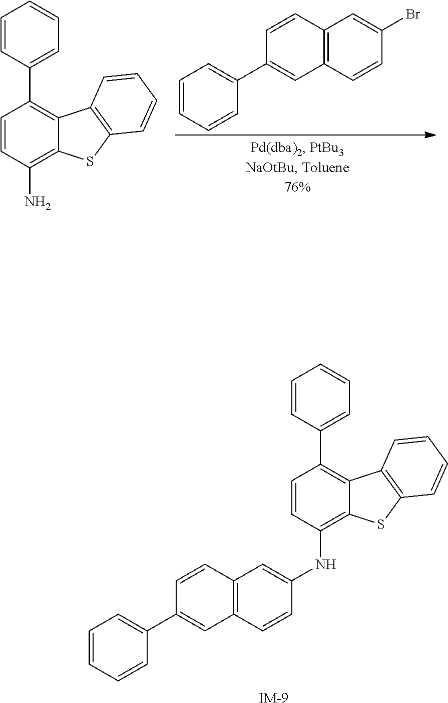

In an Ar atmosphere, in a 1,000 mL three-neck flask, Compound IM-8 (10.00 g, 36.31 mmol), Pd(dba)₂ (1.04 g, 0.05 equiv, 1.82 mmol), NaO^{t}Bu (3.49 g, 1.0 equiv, 19.3 mmol), toluene (360 mL), 2-bromo-6-phenylnaphthalene (10.28 g, 1.0 equiv, 19.3 mmol), and P^{t}Bu₃ (1.47 g, 0.2 equiv, 7.26 mmol) were sequentially added, and then heated and stirred at about 80° C. After the reaction solution was air-cooled to room temperature, organic layers were separated and obtained by adding water to the reaction solvent. The organic layers were further extracted by adding toluene to a water layer, and then were washed with saline and dried over MgSO₄. MgSO₄ was filtered and the organic layers were concentrated to obtain a crude product. The obtained crude product was purified to obtain Compound IM-9 (13.18 g, yield 76%).

By measuring FAB-MS, a mass number of m/z=477 was observed by molecular ion peak, thereby identifying Compound IM-9.

### Synthesis of Compound AC15

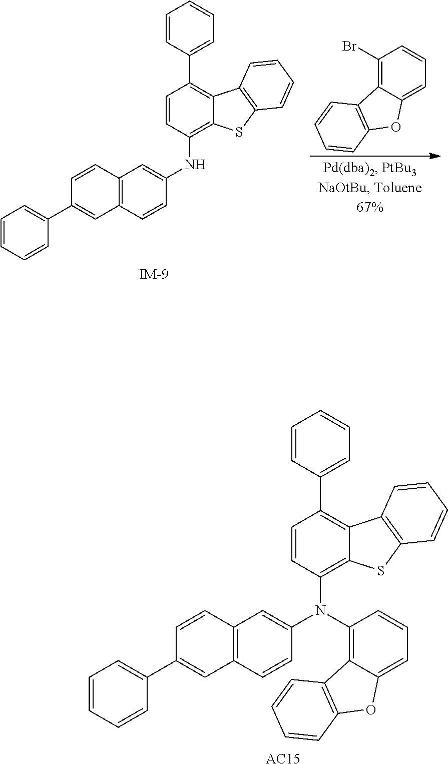

In an Ar atmosphere, in a 1,000 mL three-neck flask, Compound IM-9 (13.18 g, 27.59 mmol), Pd(dba)₂ (0.79 g, 0.05 equiv, 1.4 mmol), NaO^{t}Bu (2.92 g, 1.0 equiv, 30.3 mmol), toluene (280 mL), 1-bromodibenzo[b,d]furan (6.82 g, 1.0 equiv, 27.6 mmol), and P^{t}Bu₃ (1.12 g, 0.2 equiv, 5.52 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then were washed with saline and dried over MgSO₄. MgSO₄ was filtered and the organic layers were concentrated to obtain a crude product. The obtained crude product was purified to obtain Compound AC15 (11.88 g, yield 67%).

By measuring FAB-MS, a mass number of m/z=643 was observed by molecular ion peak, thereby identifying Compound AC15.

### (5) Synthesis of Compound AM3

Amine Compound AM3 according to an example may be synthesized by, for example, the reaction below.

### Synthesis of Compound IM-10

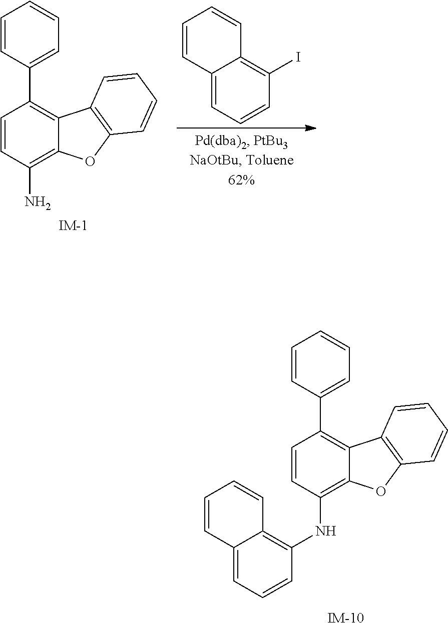

In an Ar atmosphere, in a 500 mL three-neck flask, Compound IM-1 (5.00 g, 19.3 mmol), Pd(dba)₂ (0.55 g, 0.05 equiv, 0.96 mmol), NaO^{t}Bu (1.85 g, 1.0 equiv, 19.3 mmol), toluene (190 mL), 1-iodonaphthalene (4.90 g, 1.0 equiv, 19.3 mmol), and P^{t}Bu₃ (0.78 g, 0.2 equiv, 3.9 mmol) were sequentially added, and then heated and stirred at about 80° C. After the reaction solution was air-cooled to room temperature, organic layers were separated and obtained by adding water to the reaction solvent. The organic layers were further extracted by adding toluene to a water layer, and then were washed with saline and dried over MgSO₄. MgSO₄ was filtered and the organic layers were concentrated to obtain a crude product. The obtained crude product was purified to obtain Compound IM-10 (4.63 g, yield 62%).

By measuring FAB-MS, a mass number of m/z=385 was observed by molecular ion peak, thereby identifying Compound IM-10.

### Synthesis of Compound AM3

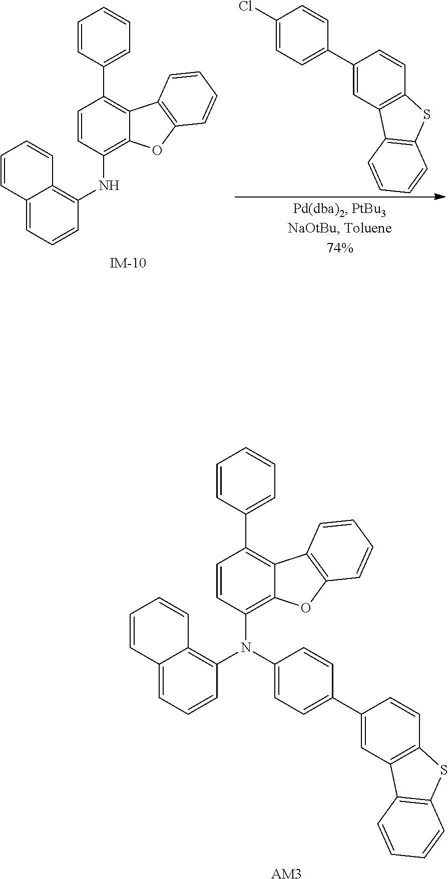 [SMILES: c1ccc(-c2ccc(Nc3cccc4ccccc34)c3oc4ccccc4c23)cc1 ; Clc1ccc(-c2ccc3sc4ccccc4c3c2)cc1 ; "IM-10" ; c1ccc(-c2ccc(N(c3ccc(-c4ccc5sc6ccccc6c5c4)cc3)c3cccc4ccccc34)c3oc4ccccc4c23)cc1 ; "AM3"]

In an Ar atmosphere, in a 500 mL three-neck flask, Compound IM-10 (4.63 g, 12.0 mmol), Pd(dba)₂ (0.35 g, 0.05 equiv, 0.60 mmol), NaO^{t}Bu (1.27 g, 1.0 equiv, 13.2 mmol), toluene (120 mL), 2-(4-chlorophenyl)dibenzo[b,d]thiophene (3.54 g, 1.0 equiv, 12.0 mmol), and P^{t}Bu₃ (0.49 g, 0.2 equiv, 2.4 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then were washed with saline and dried over MgSO₄. MgSO₄ was filtered and the organic layers were concentrated to obtain a crude product. The obtained crude product was purified to obtain Compound AM3 (5.75 g, yield 74%).

By measuring FAB-MS, a mass number of m/z=643 was observed by molecular ion peak, thereby identifying Compound AM3.

### (6) Synthesis of Compound AX12

Amine Compound AX12 according to an example may be synthesized by, for example, the reaction below.

### Synthesis of Compound IM-11

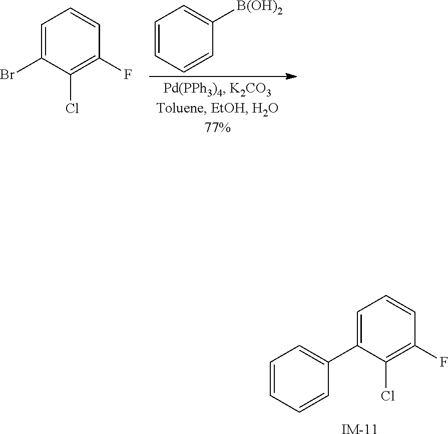

In an Ar atmosphere, in a 2,000 mL three-neck flask, 1-bromo-2-chloro-3-fluorobenzene (30.00 g, 143.2 mmol), phenylboronic acid (19.21 g, 1.1 equiv, 157.6 mmol), Pd(PPh₃)₄ (16.55 g, 0.10 equiv, 14.32 mmol), K₂CO₃ (39.59 g, 2.0 equiv, 286.5 mmol), toluene (570 mL), EtOH (280 mL), and H₂O (140 mL) were sequentially added, and then heated and stirred under reflux. After the reaction solution was air-cooled to room temperature, organic layers were separated and obtained by adding water and toluene to the reaction solvent. The organic layers were washed with saline, and then dried over MgSO₄. MgSO₄ was filtered and the organic layers were concentrated to obtain a crude product. The obtained crude product was purified to obtain Compound IM-11 (22.80 g, yield 77%).

By measuring FAB-MS, a mass number of m/z=206 was observed by molecular ion peak, thereby identifying Compound IM-11.

### Synthesis of Compound IM-12

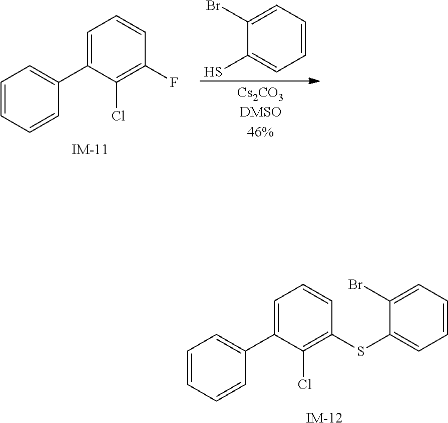

In an Ar atmosphere, in a 300 mL three-neck flask, Compound IM-11 (22.80 g, 110.3 mmol), Cs₂CO₃ (71.90 g, 2.0 equiv, 220.7 mmol), DMSO (110 mL), and 2-bromobenzenethiol (41.72 g, 2.0 equiv, 220.7 mmol) were sequentially added, and then heating and stirred at about 120° C. After the reaction solution was air-cooled to room temperature, organic layers were separated and obtained by adding water and toluene to the reaction solvent. The organic layers were washed with saline, and dried over MgSO₄. MgSO₄ was filtered and the organic layers were concentrated to obtain a crude product. The obtained crude product was purified to obtain Compound IM-12 (19.07 g, yield 46%).

By measuring FAB-MS, a mass number of m/z=374 was observed by molecular ion peak, thereby identifying Compound IM-12.

### Synthesis of Compound IM-13

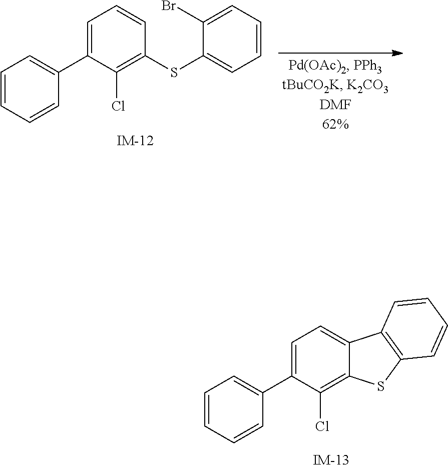

In an Ar atmosphere, in a 1,000 mL three-neck flask, Compound IM-12 (19.07 g, 50.76 mmol), tBuCO₂K (7.12 g, 1.0 equiv, 50.8 mmol), Pd(Oac)₂ (2.28 g, 0.2 equiv, 10.2 mmol), Pph₃ (2.66 g, 0.2 equiv, 10.2 mmol), K₂CO₃ (21.05 g, 3.0 equiv, 152.3 mmol), and DMSO (250 mL) were sequentially added, and then heating and stirred at about 120° C. After the reaction solution was air-cooled to room temperature, organic layers were separated and obtained by adding water and toluene to the reaction solvent. The organic layers were washed with saline, and dried over MgSO₄. MgSO₄ was filtered and the organic layers were concentrated to obtain a crude product. The obtained crude product was purified to obtain Compound IM-13 (9.28 g, yield 62%).

By measuring FAB-MS, a mass number of m/z=294 was observed by molecular ion peak, thereby identifying Compound IM-13.

### Synthesis of Compound IM-14

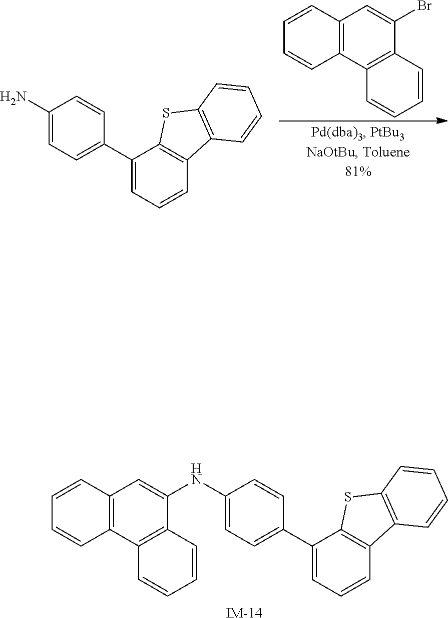

In an Ar atmosphere, in a 1,000 mL three-neck flask, 4-(dibenzo[b,d]thiophen-4-yl)aniline (15.00 g, 54.47 mmol), Pd(dba)₂ (1.57 g, 0.05 equiv, 2.72 mmol), NaO^{t}Bu (5.23 g, 1.0 equiv, 54.5 mmol), toluene (500 mL), 9-bromophenanthrene (14.01 g, 1.0 equiv, 54.47 mmol), and P^{t}Bu₃ (2.20 g, 0.2 equiv, 10.9 mmol) were sequentially added, and then heated and stirred at about 80° C. After the reaction solution was air-cooled to room temperature, organic layers were separated and obtained by adding water to the reaction solvent. The organic layers were further extracted by adding toluene to a water layer, and then were washed with saline and dried over MgSO₄. MgSO₄ was filtered and the organic layers were concentrated to obtain a crude product. The obtained crude product was purified to obtain Compound IM-14 (19.91 g, yield 81%).

By measuring FAB-MS, a mass number of m/z=451 was observed by molecular ion peak, thereby identifying Compound IM-14.

### Synthesis of Compound AX12

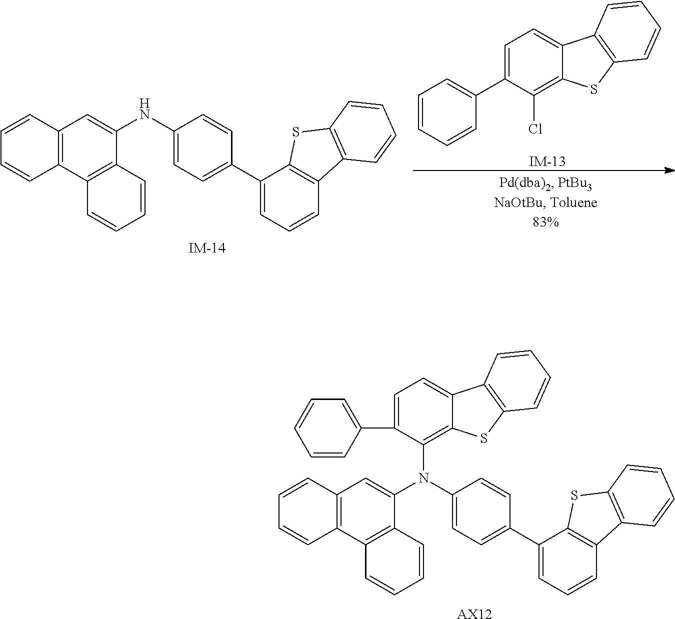

In an Ar atmosphere, in a 500 mL three-neck flask, Compound IM-14 (6.22 g, 13.8 mmol), Pd(dba)₂ (0.40 g, 0.05 equiv, 0.69 mmol), NaO^{t}Bu (1.46 g, 1.1 equiv, 15.2 mmol), toluene (140 mL), Compound IM-13 (4.06 g, 1.0 equiv, 13.8 mmol), and P^{t}Bu₃ (0.56 g, 0.2 equiv, 2.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then were washed with saline and dried over MgSO₄. MgSO₄ was filtered and the organic layers were concentrated to obtain a crude product. The obtained crude product was purified to obtain Compound AX12 (8.14 g, yield 83%).

By measuring FAB-MS, a mass number of m/z=709 was observed by molecular ion peak, thereby identifying Compound AX12.

### 2. Manufacture and Evaluation of Light Emitting Element

The light emitting element of an embodiment including the amine compound of an embodiment in a hole transport layer was manufactured as follows. Amine compounds of Compound A10, Compound D2, Compound 17, Compound AC15, Compound AM3, and Compound AX12, which are Example Compounds as described above, were used as a hole transport layer material to manufacture the light emitting elements of Examples 1 to Embodiment 6, respectively. Comparative Examples 1 to 19 correspond to the light emitting elements manufactured by using Comparative Example Compounds R1 to R19 as a hole transport layer material.

### Example Compounds

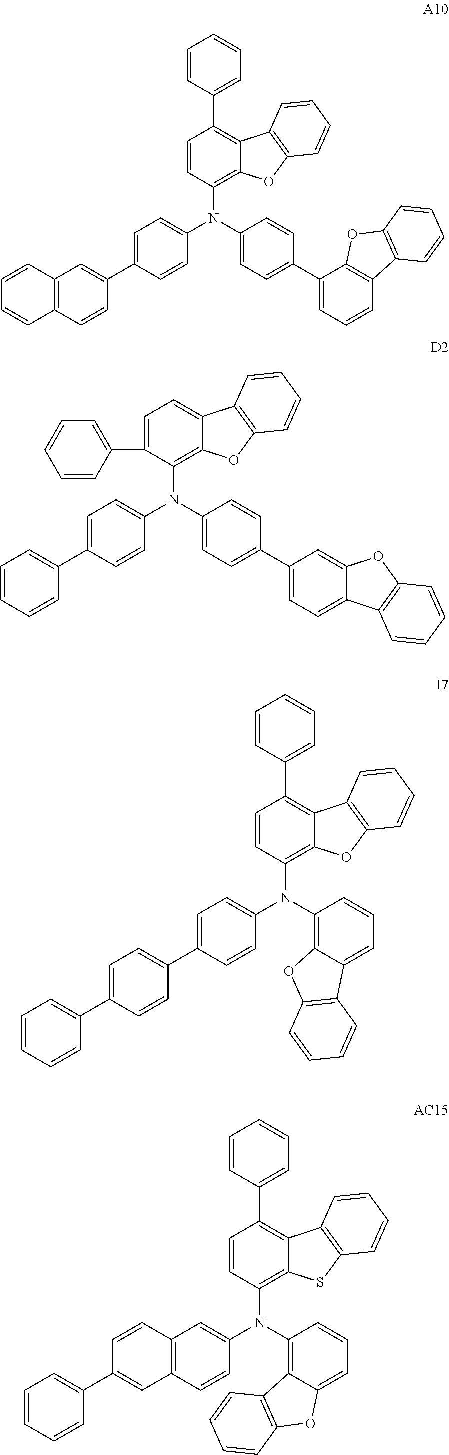 [SMILES: c1ccc(-c2ccc(N(c3ccc(-c4ccc5ccccc5c4)cc3)c3ccc(-c4cccc5c4oc4ccccc45)cc3)c3oc4ccccc4c23)cc1 ; c1ccc(-c2ccc(N(c3ccc(-c4ccc5c(c4)oc4ccccc45)cc3)c3c(-c4ccccc4)ccc4c3oc3ccccc34)cc2)cc1 ; c1ccc(-c2ccc(-c3ccc(N(c4cccc5c4oc4ccccc45)c4ccc(-c5ccccc5)c5c4oc4ccccc45)cc3)cc2)cc1 ; c1ccc(-c2ccc3cc(N(c4ccc(-c5ccccc5)c5c4sc4ccccc45)c4cccc5oc6ccccc6c45)ccc3c2)cc1]

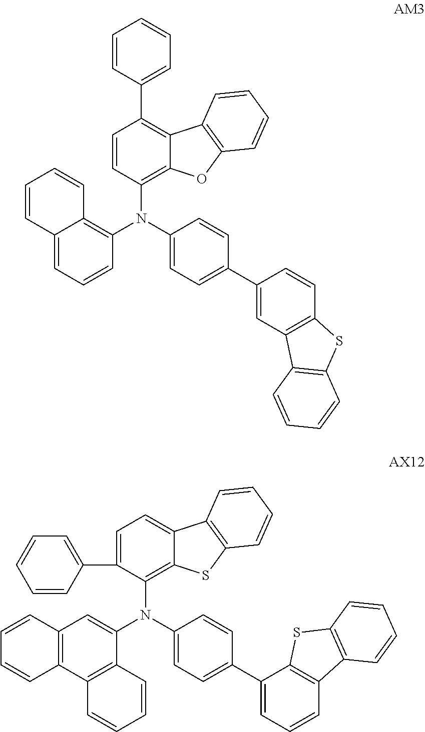 [SMILES: c1ccc(-c2ccc(N(c3ccc(-c4ccc5sc6ccccc6c5c4)cc3)c3cccc4ccccc34)c3oc4ccccc4c23)cc1 ; c1ccc(-c2ccc3c(sc4ccccc43)c2N(c2ccc(-c3cccc4c3sc3ccccc34)cc2)c2cc3ccccc3c3ccccc23)cc1]

### Comparative Example Compounds

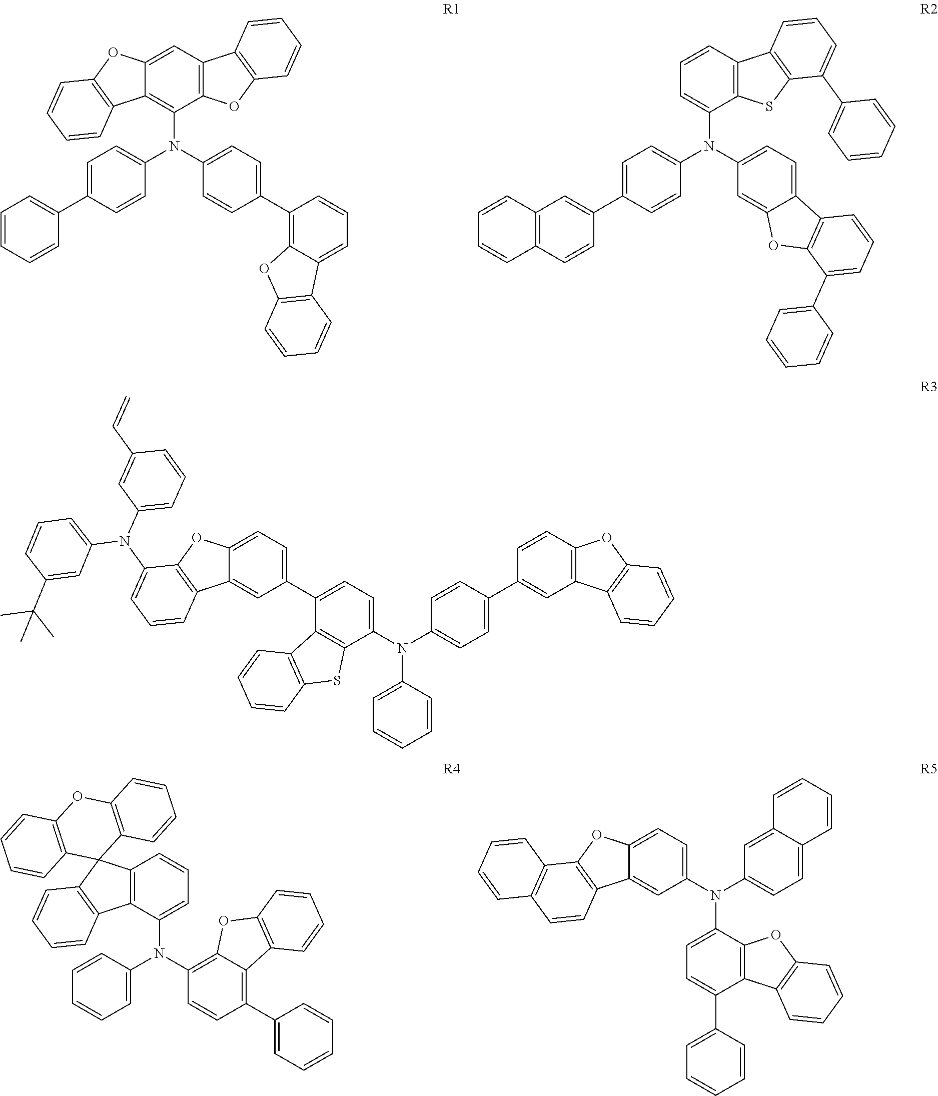 [SMILES: c1ccc(-c2ccc(N(c3ccc(-c4cccc5c4oc4ccccc45)cc3)c3c4oc5ccccc5c4cc4oc5ccccc5c34)cc2)cc1 ; c1ccc(-c2cccc3c2oc2cc(N(c4ccc(-c5ccc6ccccc6c5)cc4)c4cccc5c4sc4c(-c6ccccc6)cccc45)ccc23)cc1 ; C=Cc1cccc(N(c2cccc(C(C)(C)C)c2)c2cccc3c2oc2ccc(-c4ccc(N(c5ccccc5)c5ccc(-c6ccc7oc8ccccc8c7c6)cc5)c5sc6ccccc6c45)cc23)c1 ; c1ccc(-c2ccc(N(c3ccccc3)c3cccc4c3-c3ccccc3C43c4ccccc4Oc4ccccc43)c3oc4ccccc4c23)cc1 ; c1ccc(-c2ccc(N(c3ccc4ccccc4c3)c3ccc4oc5c6ccccc6ccc5c4c3)c3oc4ccccc4c23)cc1]

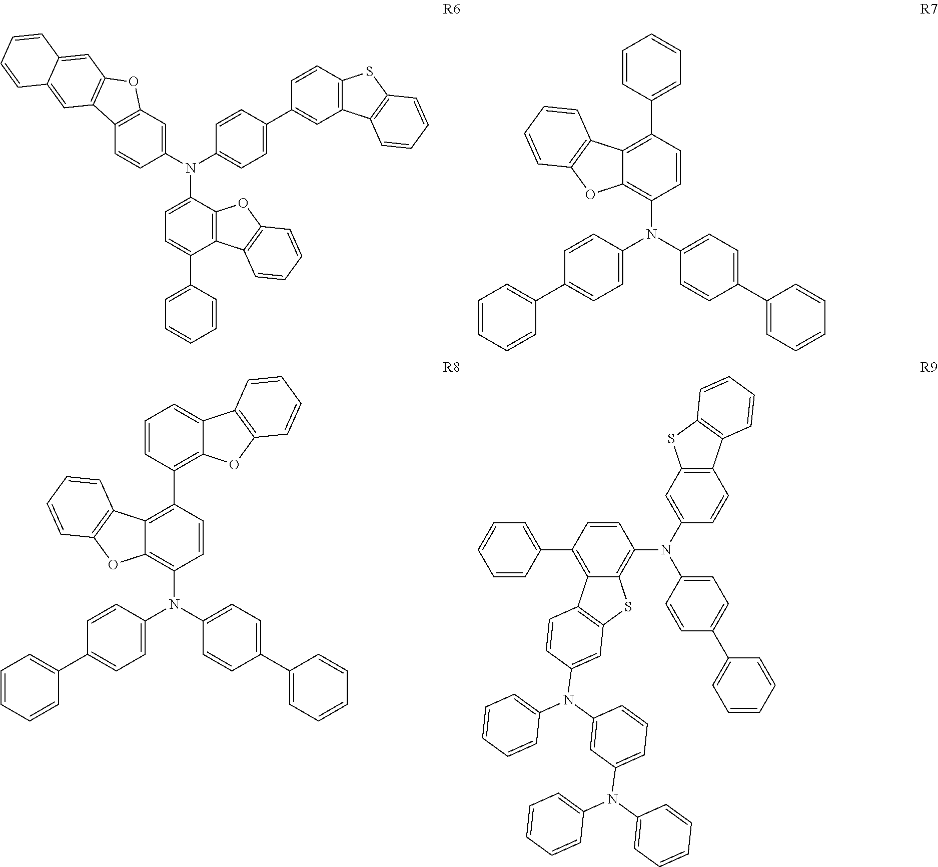 [SMILES: c1ccc(-c2ccc(N(c3ccc(-c4ccc5sc6ccccc6c5c4)cc3)c3ccc4c(c3)oc3cc5ccccc5cc34)c3oc4ccccc4c23)cc1 ; c1ccc(-c2ccc(N(c3ccc(-c4ccccc4)cc3)c3ccc(-c4ccccc4)c4c3oc3ccccc34)cc2)cc1 ; c1ccc(-c2ccc(N(c3ccc(-c4ccccc4)cc3)c3ccc(-c4cccc5c4oc4ccccc45)c4c3oc3ccccc34)cc2)cc1 ; c1ccc(-c2ccc(N(c3ccc4c(c3)sc3ccccc34)c3ccc(-c4ccccc4)c4c3sc3cc(N(c5ccccc5)c5cccc(N(c6ccccc6)c6ccccc6)c5)ccc34)cc2)cc1]

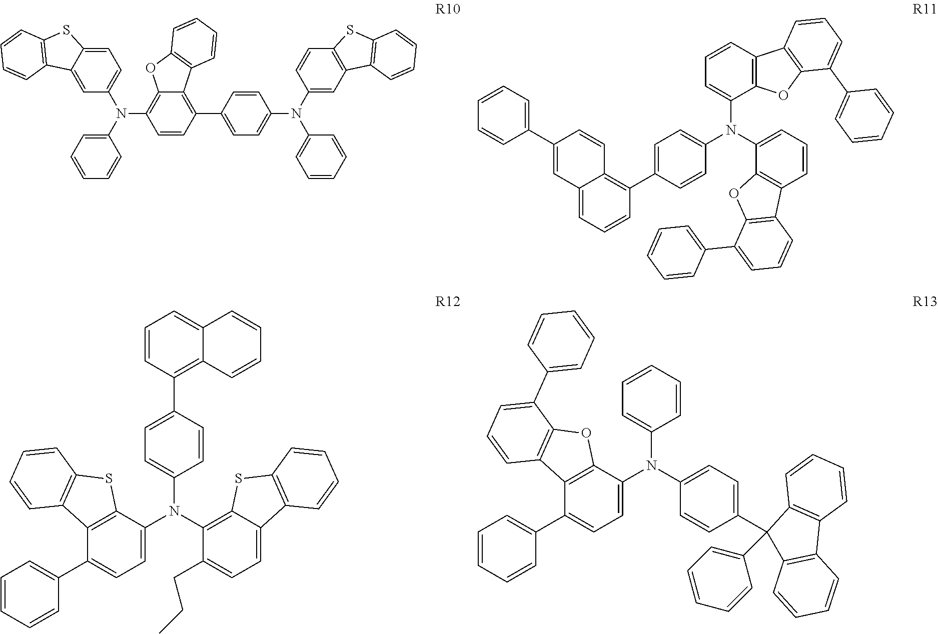 [SMILES: c1ccc(N(c2ccc(-c3ccc(N(c4ccccc4)c4ccc5sc6ccccc6c5c4)c4oc5ccccc5c34)cc2)c2ccc3sc4ccccc4c3c2)cc1 ; c1ccc(-c2ccc3c(-c4ccc(N(c5cccc6c5oc5c(-c7ccccc7)cccc56)c5cccc6c5oc5c(-c7ccccc7)cccc56)cc4)cccc3c2)cc1 ; CCCc1ccc2c(sc3ccccc32)c1N(c1ccc(-c2cccc3ccccc23)cc1)c1ccc(-c2ccccc2)c2c1sc1ccccc12 ; c1ccc(-c2cccc3c2oc2c(N(c4ccccc4)c4ccc(C5(c6ccccc6)c6ccccc6-c6ccccc65)cc4)ccc(-c4ccccc4)c23)cc1]

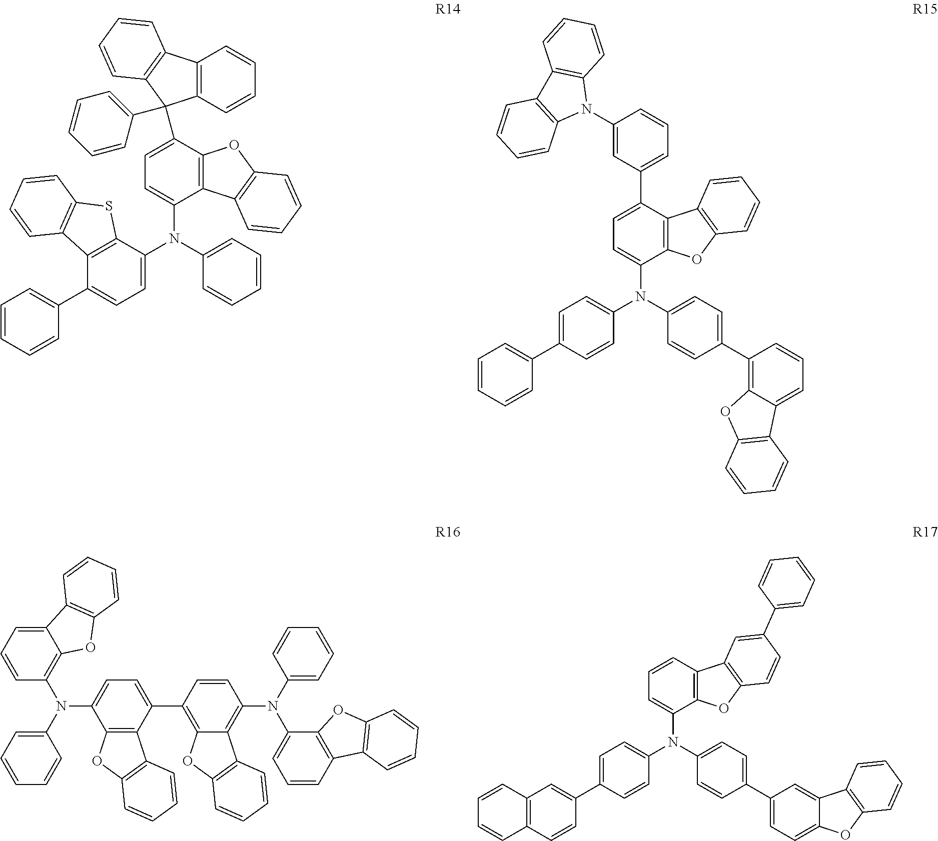 [SMILES: c1ccc(-c2ccc(N(c3ccccc3)c3ccc(C4(c5ccccc5)c5ccccc5-c5ccccc54)c4oc5ccccc5c34)c3sc4ccccc4c23)cc1 ; c1ccc(-c2ccc(N(c3ccc(-c4cccc5c4oc4ccccc45)cc3)c3ccc(-c4cccc(-n5c6ccccc6c6ccccc65)c4)c4c3oc3ccccc34)cc2)cc1 ; c1ccc(N(c2cccc3c2oc2ccccc23)c2ccc(-c3ccc(N(c4ccccc4)c4cccc5c4oc4ccccc45)c4c3oc3ccccc34)c3c2oc2ccccc23)cc1 ; c1ccc(-c2ccc3oc4c(N(c5ccc(-c6ccc7ccccc7c6)cc5)c5ccc(-c6ccc7oc8ccccc8c7c6)cc5)cccc4c3c2)cc1]

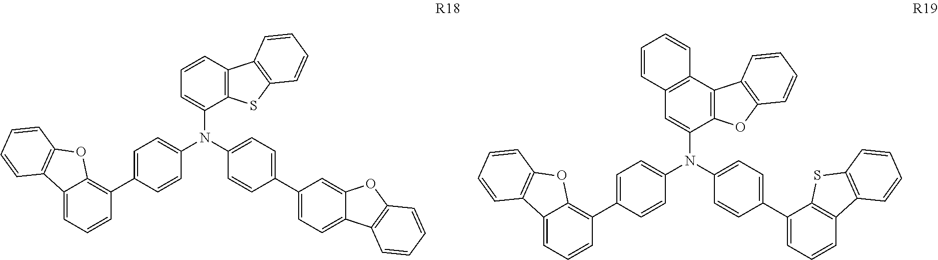 [SMILES: c1ccc2c(c1)oc1cc(-c3ccc(N(c4ccc(-c5cccc6c5oc5ccccc56)cc4)c4cccc5c4sc4ccccc45)cc3)ccc12 ; c1ccc2c(c1)cc(N(c1ccc(-c3cccc4c3oc3ccccc34)cc1)c1ccc(-c3cccc4c3sc3ccccc34)cc1)c1oc3ccccc3c12]

### Manufacture of Light Emitting Element

An ITO glass substrate of about 15 Ω/cm² (about 1,200 Å) from Corning Co. was cut to a size of 50 mm×50 mm×0.7 mm, washed with isopropyl alcohol and ultrapure water, and cleansed by ultrasonic waves for about 5 minutes, and then irradiated with ultraviolet rays for about 30 minutes and treated with ozone. Then, (4,4′,4″-tris{N,-(2-naphthyl)-N-phenylamino}-triphenylamine (2-TNATA) was deposited in vacuum to form a 600 Å-thick hole injection layer, and then the Example Compound or Comparative Example Compound was deposited in vacuum to form a 300 Å-thick hole transport layer.

On the hole transport layer, 9,10-di(naphthalen-2-yl)anthracene (ADN) as a blue fluorescent host and 2,5,8,11-Tetra-t-butylperylene (TBP) as a fluorescent dopant were co-deposited in a ratio of 97:3 to form a 250 Å-thick emission layer.

On the emission layer, a 250 Å-thick electron transport layer was formed with tris(8-hydroxyquinolino)aluminum (Alq₃), and then LiF was deposited to form a 10 Å-thick electron injection layer. On the electron injection layer, a 1,000 Å-thick second electrode was formed with aluminum (Al).

In addition, compounds of each functional layer used to manufacture light emitting elements are as follows:

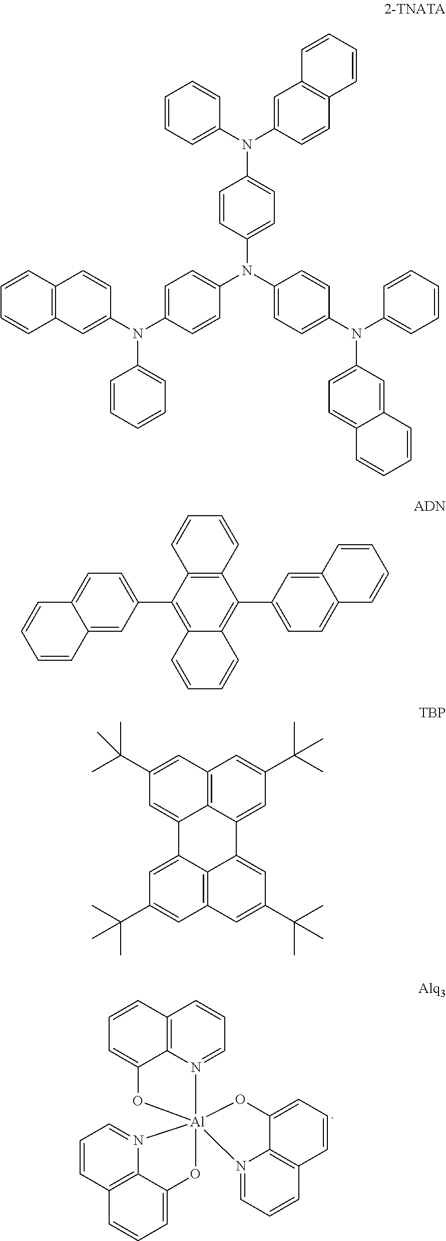

### Evaluation of Light Emitting Element

Evaluation results of the light emitting elements in Examples 1 to 6 and Comparative Examples 1 to 19 are listed in Table 1. Brightnesses, luminous efficiencies, and half lives of the light emitting elements are listed in Table 1 below.

In the characteristic evaluation results of Examples and Comparative Examples listed in Table 1, the maximum luminous efficiency represents an efficiency value measured at a current density of 10 mA/cm². The half life represents a half life value obtained by measuring, at a current density of 1.0 mA/cm², a point at which an initial brightness, 100 cd/m², is reduced by half. In the case of the maximum luminous efficiency and half life, the maximum luminous efficiency and half life in Comparative Example 7 are set as 100%, a reference value, and relative values are indicated as a numerical value by %. The evaluation of the current density and luminous efficiency of the light emitting element was performed in a dark room using a 2400 Series Source Meter from Keithley Instruments Inc., a Color and Luminance Meter CS-200 from Konica Minolta, Inc., and PC Program LabVIEW 8.2 for the measurement from Japan National Instrument, Inc.

TABLE 1

| | | Maximum | |
|---|---|---|---|
| | Hole transport layer | luminous | |
| | material | Efficiency | Half life |
| Example 1 | Example Compound A10 | 125% | 134% |
| Example 2 | Example Compound D2 | 121% | 126% |
| Example 3 | Example Compound I7 | 129% | 117% |
| Example 4 | Example Compound AC15 | 118% | 121% |
| Example 5 | Example Compound AM3 | 127% | 115% |
| Example 6 | Example Compound AX12 | 117% | 123% |
| Comparative | Comparative Example | 104% | 88% |
| Example 1 | Compound R1 | | |
| Comparative | Comparative Example | 102% | 85% |
| Example 2 | Compound R2 | | |
| Comparative | Comparative Example | 109% | 64% |
| Example 3 | Compound R3 | | |
| Comparative | Comparative Example | 96% | 79% |
| Example 4 | Compound R4 | | |
| Comparative | Comparative Example | 107% | 88% |
| Example 5 | Compound R5 | | |
| Comparative | Comparative Example | 111% | 82% |
| Example 6 | Compound R6 | | |
| Comparative | Comparative Example | 100% | 100% |
| Example 7 | Compound R7 | | |
| Comparative | Comparative Example | 104% | 94% |
| Example 8 | Compound R8 | | |
| Comparative | Comparative Example | 107% | 69% |
| Example 9 | Compound R9 | | |
| Comparative | Comparative Example | 102% | 74% |
| Example 10 | Compound R10 | | |
| Comparative | Comparative Example | 104% | 80% |
| Example 11 | Compound R11 | | |
| Comparative | Comparative Example | 99% | 81% |
| Example 12 | Compound R12 | | |
| Comparative | Comparative Example | 96% | 88% |
| Example 13 | Compound R13 | | |
| Comparative | Comparative Example | 103% | 90% |
| Example 14 | Compound R14 | | |
| Comparative | Comparative Example | 103% | 82% |
| Example 15 | Compound R15 | | |
| Comparative | Comparative Example | 108% | 63% |
| Example 16 | Compound R16 | | |
| Comparative | Comparative Example | 113% | 91% |
| Example 17 | Compound R17 | | |
| Comparative | Comparative Example | 109% | 83% |
| Example 18 | Compound R18 | | |
| Comparative | Comparative Example | 102% | 79% |
| Example 19 | Compound R19 | | |

Referring to the results of Table 1, it may be seen that Examples of the light emitting elements in which the amine compounds according to examples of the present disclosure are used as a hole transport layer material exhibit relatively high luminous efficiencies and long element service lives compared to the Comparative Examples.

The Example Compounds are tertiary amine compounds including the first, second, and third substituents which are linked to the core nitrogen atom, and includes the first substituent having a dibenzoheterol structure which is linked at the fourth carbon position to the core nitrogen atom, so that an oxygen atom and a nitrogen atom may be bonded at the ortho-position, thereby the charge density may increase, and thus the hole transport ability may be improved, leading to the improvement of the efficiency. In addition, the first substituent is substituted with an aryl group or a heteroaryl group at the first or third carbon position, and thus the conjugated structure may be increased, thereby the stability in the molecular structure may be improved, and thus the element service life may be improved when the first substituent is applied to the light emitting element. In addition, the first substituent is substituted with an aryl group or a heteroaryl group at the first or third carbon position, and thus intermolecular stacking is likely to occur, thereby the distance between the molecules may narrow, and thus the hole transport ability may be improved. The amine compound of an embodiment further includes the second substituent having a dibenzoheterol structure, and thus the hole transport ability may further be improved, and the stability in a radical cation state may be improved. Therefore, it may be expected that the elements in Examples including the Compounds of the examples as a hole transport layer material exhibit high luminous efficiency and long element service life.

Comparative Example 1 exhibited the results that the element service life and efficiency were reduced compared to Examples 1 to 6. It is thought that the luminous efficiency and service life were reduced when Comparative Example Compound R1 was applied to the element because the deposition temperature of the compound is relatively high due to the planarity of the benzobisbenzofuranyl moiety included in the Comparative Example Compound R1 and thus the stability of the compound is deteriorated, for example, the compound is decomposed during deposition.

Comparative Examples 2, 11, 17, and 18 exhibited the results that the element service life and efficiency were reduced compared to Examples 1 to 6. It is thought that Comparative Examples Compounds R2, R11, R17, and R18, unlike the Example Compounds, have reduced luminous efficiencies and service lives compared to the elements of Examples because an aryl group or a heteroaryl group is not substituted at the first or third carbon position at the corresponding position of the first substituent and thus the conjugated structure of the molecule is not increased.

Comparative Examples 3, 9, 10, and 16 exhibited the results that the element service life were particularly reduced compared to Examples 1 to 6. It is thought that each of Comparative Example Compounds R3, R9, R10, and R16 has a diamine or triamine structure having a plurality of amine groups in the molecular structure, and thus has high hole transport ability, but has high reactivity with the surrounding molecules, and thus deterioration occurs when the element is driven, thereby reducing luminous efficiency and service life.

Comparative Example 4 exhibited the results that the element service life and efficiency were reduced compared to Examples 1 to 6. It is thought that the luminous efficiency and service life were reduced when Comparative Example Compound R4 was applied to the element because Comparative Example Compound R4 has a spiro[9H-fluorene-9,9′-[9H]xanthene] substituent structure containing a fluorene moiety, and the structure has a quaternary carbon, and the position of the quaternary carbon is a position in which a bond is easily broken by heat, and thus the stability of the compound is deteriorated, for example, the compound is decomposed during deposition.

Comparative Examples 5, 6, and 19 exhibited the results that the element service life and efficiency were reduced compared to Examples 1 to 6. It is thought that the luminous efficiencies and service lives were reduced when Comparative Example Compounds R5, R6, and R19 were applied to the elements, respectively, because each of Comparative Example Compounds R5, R6, and R19 has a benzonaphthofuran moiety or a benzonaphthothiophene moiety in the molecular structure thereof, the fused ring skeleton of the structure has a relatively high deposition temperature due to the planarity thereof, and thus the stability of the compound is deteriorated, for example, the compound is decomposed during deposition.

Comparative Examples 7 and 8 exhibited the results that the element service life and efficiency were reduced compared to Examples 1 to 6. In embodiments of the present disclosure, in addition to the first substituent having a dibenzoheterol structure having a substituent at the first carbon position or the third carbon position, the second substituent having a dibenzoheterol structure having lone pairs may be additionally bonded to the core nitrogen atom, thereby further improving hole transport ability. In addition, the oxygen atom may stabilize a radical cation state, and thus the service life may be improved when the compound is applied to the element. It is thought that Comparative Compound R7 used in the element of Comparative Example 7 does not include an additional dibenzoheterol substituent corresponding to the second substituent, and Comparative Compound R8 used in the element of Comparative Example 8 includes two dibenzofuranyl groups in the molecule, but is not in the form in which two dibenzoheterol groups are each bonded to the core nitrogen atom like the Example Compounds, and thus, the luminous efficiencies and service lives were reduced when the compounds were applied to the elements.

Comparative Example 12 exhibited the results that the element service life and efficiency were reduced compared to Examples 1 to 6. It is thought that the luminous efficiency and service life were reduced when Comparative Example Compound R12 was applied to the element because Comparative Example Compound R12 has an alkyl substituent substituted in the dibenzoheterol structure corresponding to the first substituent, and the position of the alkyl substituent is a position in which a bond is easily broken by heat, and thus the stability of the compound is deteriorated, for example, the compound is decomposed during deposition.

Comparative Examples 13 and 14 exhibited the results that the element service life and efficiency were reduced compared to Examples 1 to 6. It is thought that the luminous efficiencies and service lives were reduced when Comparative Example Compounds R13 and R14 were applied to the elements, respectively, because each of Comparative Example Compounds R13 and R14 has a fluorene moiety in the molecular structure, and the fluorene moiety has a quaternary carbon, and the position of the quaternary carbon is a position in which a bond is easily broken by heat, and thus the stability of the compound is deteriorated, for example, the compound is decomposed during deposition.

Comparative Example 15 exhibited the results that the element service life was particularly reduced compared to Examples 1 to 6. It is thought that the amine compound including a carbazole group in the molecular structure like Comparative Example Compound R15 has high hole transport ability, but has high reactivity with the surrounding molecules, and thus the element service life is reduced due to the deterioration during driving of the element.

It may be confirmed that when the Comparative Example Compounds are applied to the light emitting elements, the brightness, luminous efficiency, and half life are reduced compared with Example Compounds. That is, referring to Table 1, the light emitting elements using the amine compounds according to examples of the present disclosure exhibit improved element characteristics in the luminous efficiency or element service life as compared to Comparative Examples.

The light emitting element of an embodiment may include the amine compound of an embodiment, thereby exhibiting high efficiency and long service life characteristics.

The amine compound of an embodiment may exhibit high efficiency and long service life characteristics when applied to a light emitting element.

Although the subject matter of the present disclosure has been described with reference to example embodiments, it will be understood that the subject matter of the present disclosure should not be limited to the disclosed embodiments but various changes and modifications can be made by those skilled in the art without departing from the spirit and scope of the present disclosure. Accordingly, the technical scope of the present disclosure is not intended to be limited to the contents set forth in the detailed description of the specification, but is intended to be defined by the appended claims, and equivalents thereof.

What is claimed is:

1. A light emitting element comprising:
a first electrode;
a second electrode on the first electrode; and
at least one functional layer which is between the first electrode and the second electrode and comprises an amine compound represented by Formula 1 below: 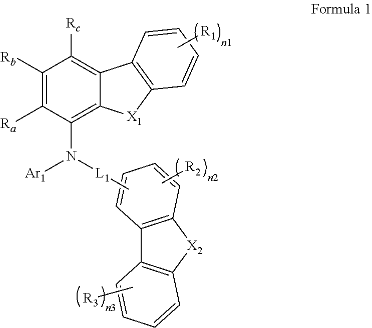
wherein, in Formula 1 above,
X₁ and X₂ are each independently O or S,
Ar is a substituted or unsubstituted aryl group having 6 to 40 ring-forming carbon atoms, or a substituted or unsubstituted heteroaryl group having 2 to 40 ring-forming carbon atoms,
Rₐ to R_{c} are each independently a hydrogen atom, a deuterium atom, a substituted or unsubstituted aryl group having 6 to 40 ring-forming carbon atoms, or a substituted or unsubstituted heteroaryl group having 2 to 40 ring-forming carbon atoms,
at least one selected from Rₐ and R_{c} is a substituted or unsubstituted aryl group having 6 to 40 ring-forming carbon atoms, or a substituted or unsubstituted heteroaryl group having 2 to 40 ring-forming carbon atoms,
R₁ to R₃ are each independently a hydrogen atom, a deuterium atom, a halogen atom, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms,
L₁ is a direct linkage, a substituted or unsubstituted arylene group having 6 to 40 ring-forming carbon atoms, or a substituted or unsubstituted heteroarylene group having 2 to 40 ring-forming carbon atoms,
the case where each of Rₐ to R_{c} comprises a carbazole group is excluded,
the case where each of Ar₁, Rₐ to R_{c}, L₁, and R₁ to R₃ comprises a fluorenyl group, a benzonaphthofuranyl group, a benzonaphthothiophenyl group, and a benzobisbenzofuranyl group is excluded,
n1 and n3 are each independently an integer of 0 to 4, and
n2 is an integer of 0 to 3.

2. The light emitting element of claim 1, wherein the at least one functional layer comprises an emission layer, a hole transport region between the first electrode and the emission layer, and an electron transport region between the emission layer and the second electrode, and
the hole transport region comprises the amine compound represented by Formula 1 above.

3. The light emitting element of claim 2, wherein the hole transport region comprises a hole injection layer on the first electrode, and a hole transport layer on the hole injection layer, and
the hole transport layer comprises the amine compound represented by Formula 1 above.

4. The light emitting element of claim 2, wherein a layer, which is adjacent to the emission layer, among the plurality of layers included in the hole transport region comprises the amine compound represented by Formula 1 above.

5. The light emitting element of claim 1, wherein the amine compound represented by Formula 1 above is a monoamine compound.

6. The light emitting element of claim 1, wherein the amine compound represented by Formula 1 above is represented by any one selected from among Formula 2-1 to Formula 2-3 below: 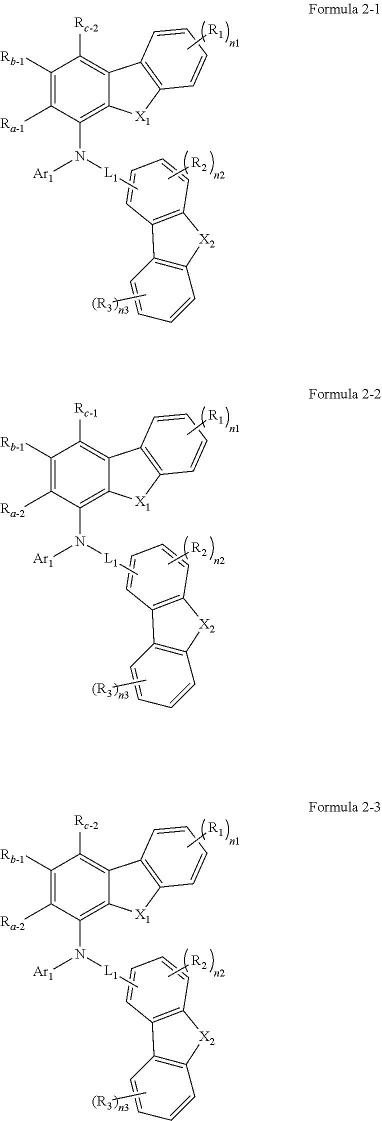
wherein, in Formula 2-1 to Formula 2-3 above,
Rₐ₋₁, R_{b-1}, and R_{c-1} are each independently a hydrogen atom or a deuterium atom,
Rₐ₋₂ and R_{c-2} are each independently a substituted or unsubstituted aryl group having 6 to 40 ring-forming carbon atoms, or a substituted or unsubstituted heteroaryl group having 2 to 40 ring-forming carbon atoms,
the case where each of Rₐ₋₂ and R_{c-2} comprises a carbazole group, a fluorenyl group, a benzonaphthofuranyl group, a benzonaphthothiophenyl group, or a benzobisbenzofuranyl group is excluded, and
X₁, X₂, Ar₁, R₁ to R₃, L₁, n1, n2, and n3 are the same as defined with respect to Formula 1 above.

7. The light emitting element of claim 1, wherein the amine compound represented by Formula 1 above is represented by any one selected from among Formula 3-1 to Formula 3-4 below: 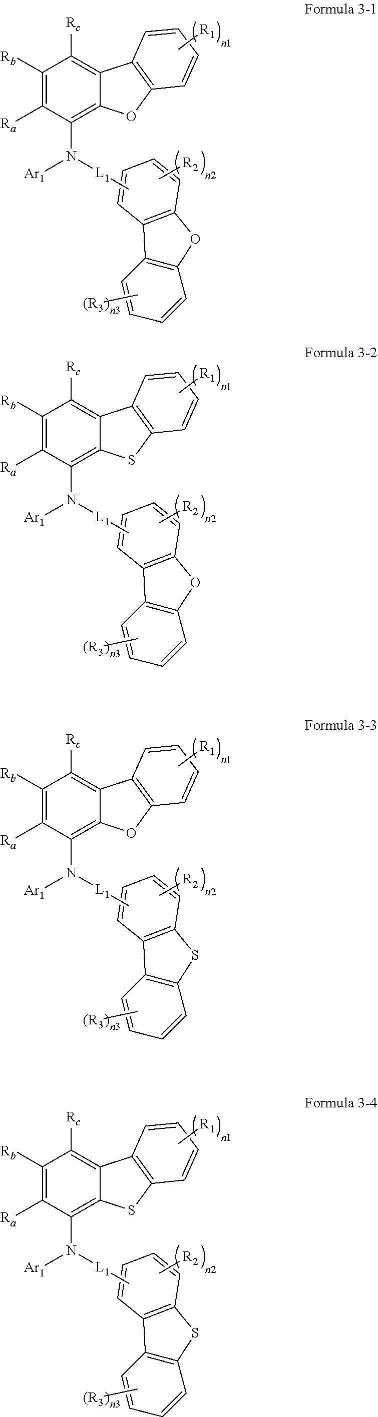
wherein, in Formula 3-1 to Formula 3-4 above,
Ar₁, Rₐ to R_{c}, R₁ to R₃, L₁, n1, n2, and n3 are the same as defined with respect to Formula 1 above.

8. The light emitting element of claim 1, wherein the amine compound represented by Formula 1 above is represented by any one selected from among Formula 4-1 to Formula 4-4 below: 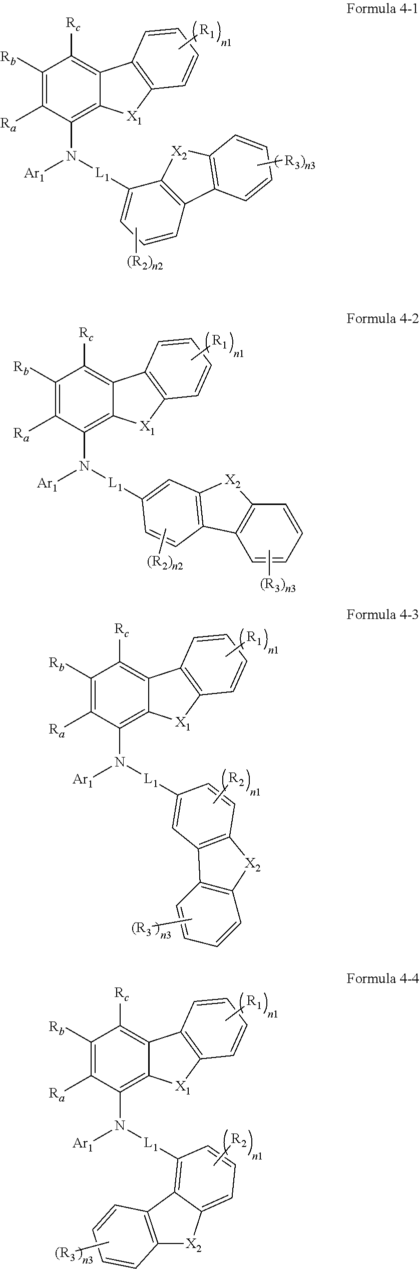
wherein, in Formula 4-1 to Formula 4-4 above,
X₁, X₂, Ar₁, Rₐ to R_{c}, R₁ to R₃, L₁, n1, n2, and n3 are the same as defined with respect to Formula 1 above.

9. The light emitting element of claim 1, wherein, in Formula 1 above,
at least one selected from Rₐ and R_{c} is a substituted or unsubstituted phenyl group, a substituted or unsubstituted naphthyl group, a substituted or unsubstituted biphenyl group, a substituted or unsubstituted phenanthrenyl group, a substituted or unsubstituted dibenzofuranyl group, or a substituted or unsubstituted dibenzothiophenyl group.

10. The light emitting element of claim 1, wherein, in Formula 1 above,
L₁ is a direct linkage, or a substituted or unsubstituted p-phenylene group.

11. The light emitting element of claim 1, wherein, in Formula 1 above,
Ar₁ is a substituted or unsubstituted phenyl group, a substituted or unsubstituted biphenyl group, a substituted or unsubstituted terphenyl group, a substituted or unsubstituted naphthyl group, a substituted or unsubstituted phenanthrene group, a substituted or unsubstituted dibenzofuranyl group, or a substituted or unsubstituted dibenzothiophenyl group.

12. The light emitting element of claim 1, wherein, in Formula 1 above,
R₂ and R₃ are each independently a hydrogen atom, a deuterium atom, a substituted or unsubstituted phenyl group.

13. The light emitting element of claim 1, wherein the amine compound represented by Formula 1 above is represented by any one selected from among compounds in Compound Group 1 below:
Compound Group 1

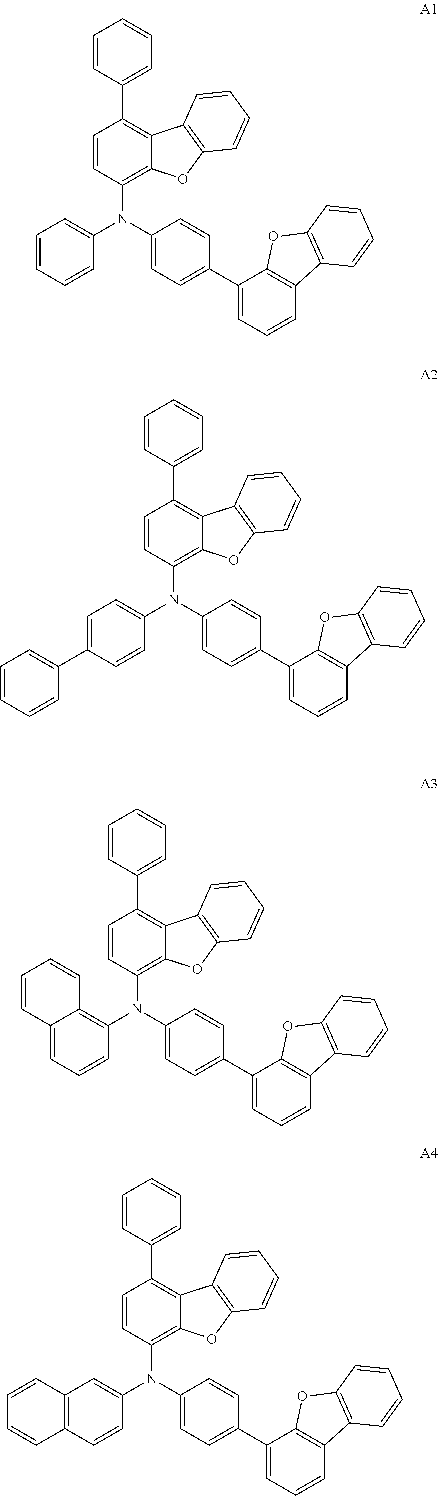 [SMILES: c1ccc(-c2ccc(N(c3ccccc3)c3ccc(-c4cccc5c4oc4ccccc45)cc3)c3oc4ccccc4c23)cc1 ; c1ccc(-c2ccc(N(c3ccc(-c4cccc5c4oc4ccccc45)cc3)c3ccc(-c4ccccc4)c4c3oc3ccccc34)cc2)cc1 ; c1ccc(-c2ccc(N(c3ccc(-c4cccc5c4oc4ccccc45)cc3)c3cccc4ccccc34)c3oc4ccccc4c23)cc1 ; c1ccc(-c2ccc(N(c3ccc(-c4cccc5c4oc4ccccc45)cc3)c3ccc4ccccc4c3)c3oc4ccccc4c23)cc1]

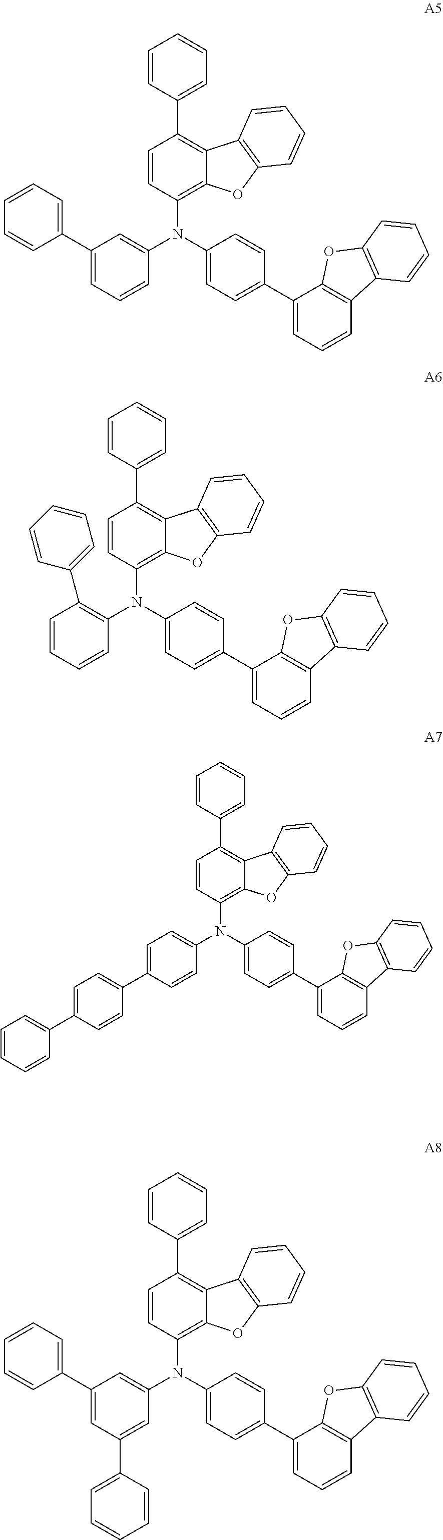 [SMILES: c1ccc(-c2cccc(N(c3ccc(-c4cccc5c4oc4ccccc45)cc3)c3ccc(-c4ccccc4)c4c3oc3ccccc34)c2)cc1 ; c1ccc(-c2ccccc2N(c2ccc(-c3cccc4c3oc3ccccc34)cc2)c2ccc(-c3ccccc3)c3c2oc2ccccc23)cc1 ; c1ccc(-c2ccc(-c3ccc(N(c4ccc(-c5cccc6c5oc5ccccc56)cc4)c4ccc(-c5ccccc5)c5c4oc4ccccc45)cc3)cc2)cc1 ; c1ccc(-c2cc(-c3ccccc3)cc(N(c3ccc(-c4cccc5c4oc4ccccc45)cc3)c3ccc(-c4ccccc4)c4c3oc3ccccc34)c2)cc1]

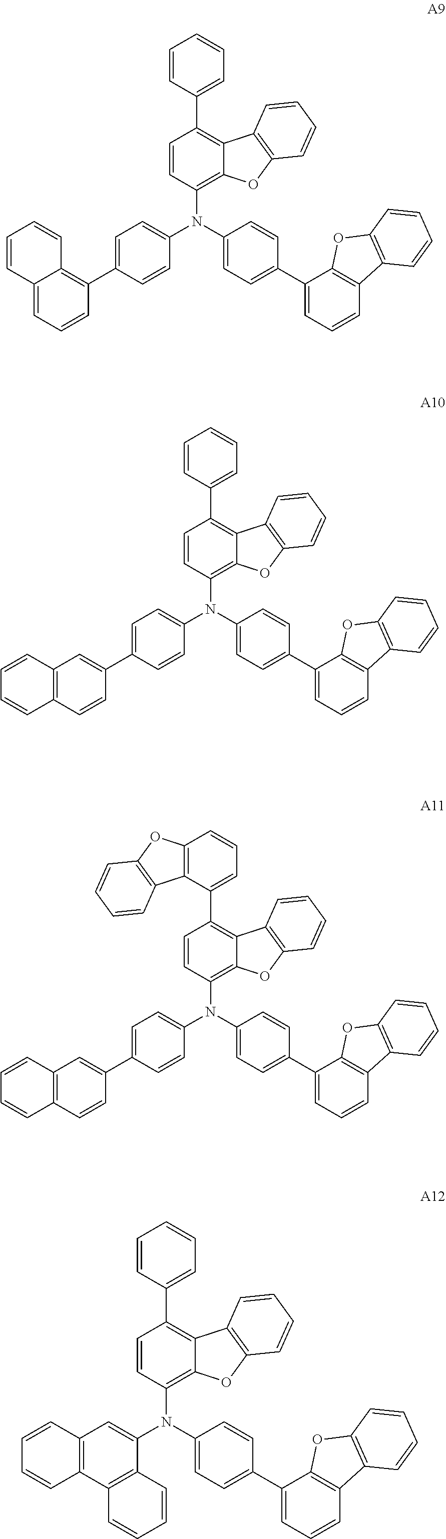 [SMILES: c1ccc(-c2ccc(N(c3ccc(-c4cccc5ccccc45)cc3)c3ccc(-c4cccc5c4oc4ccccc45)cc3)c3oc4ccccc4c23)cc1 ; c1ccc(-c2ccc(N(c3ccc(-c4ccc5ccccc5c4)cc3)c3ccc(-c4cccc5c4oc4ccccc45)cc3)c3oc4ccccc4c23)cc1 ; c1ccc2cc(-c3ccc(N(c4ccc(-c5cccc6c5oc5ccccc56)cc4)c4ccc(-c5cccc6oc7ccccc7c56)c5c4oc4ccccc45)cc3)ccc2c1 ; c1ccc(-c2ccc(N(c3ccc(-c4cccc5c4oc4ccccc45)cc3)c3cc4ccccc4c4ccccc34)c3oc4ccccc4c23)cc1]

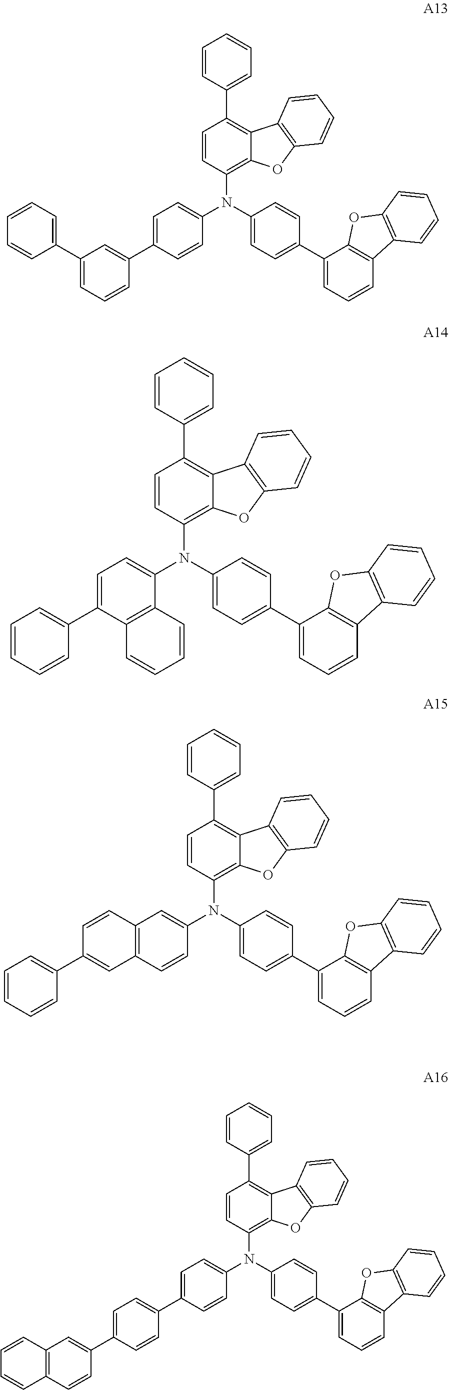 [SMILES: c1ccc(-c2cccc(-c3ccc(N(c4ccc(-c5cccc6c5oc5ccccc56)cc4)c4ccc(-c5ccccc5)c5c4oc4ccccc45)cc3)c2)cc1 ; c1ccc(-c2ccc(N(c3ccc(-c4cccc5c4oc4ccccc45)cc3)c3ccc(-c4ccccc4)c4c3oc3ccccc34)c3ccccc23)cc1 ; c1ccc(-c2ccc3cc(N(c4ccc(-c5cccc6c5oc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4cccc5c4oc4ccccc45)cc3)c3oc4ccccc4c23)cc1]

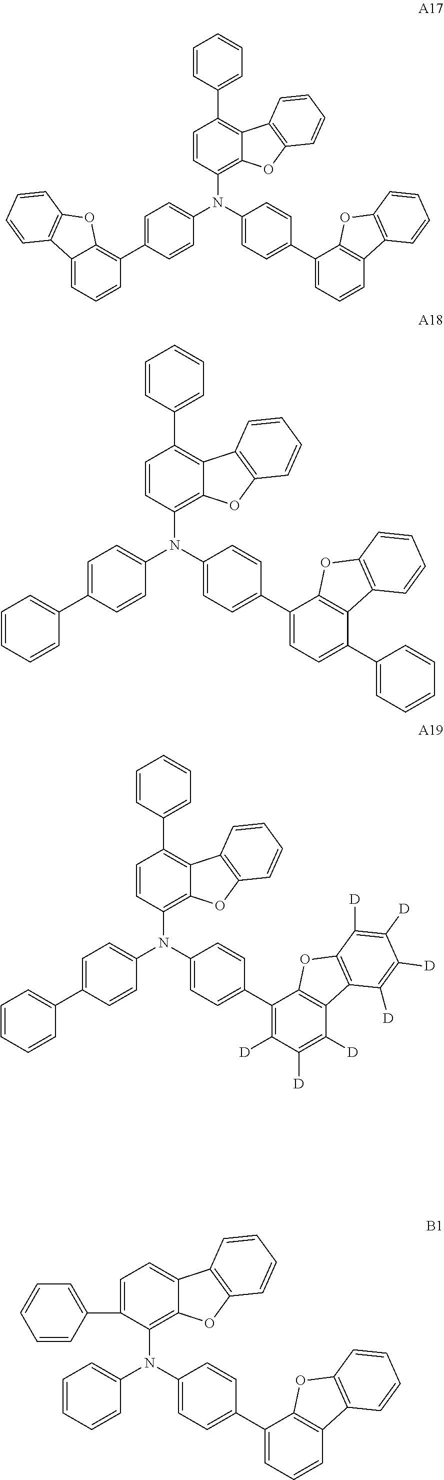 [SMILES: c1ccc(-c2ccc(N(c3ccc(-c4cccc5c4oc4ccccc45)cc3)c3ccc(-c4cccc5c4oc4ccccc45)cc3)c3oc4ccccc4c23)cc1 ; c1ccc(-c2ccc(N(c3ccc(-c4ccc(-c5ccccc5)c5c4oc4ccccc45)cc3)c3ccc(-c4ccccc4)c4c3oc3ccccc34)cc2)cc1 ; [2H]c1c([2H])c([2H])c2c(oc3c(-c4ccc(N(c5ccc(-c6ccccc6)cc5)c5ccc(-c6ccccc6)c6c5oc5ccccc56)cc4)c([2H])c([2H])c([2H])c32)c1[2H] ; c1ccc(-c2ccc3c(oc4ccccc43)c2N(c2ccccc2)c2ccc(-c3cccc4c3oc3ccccc34)cc2)cc1]

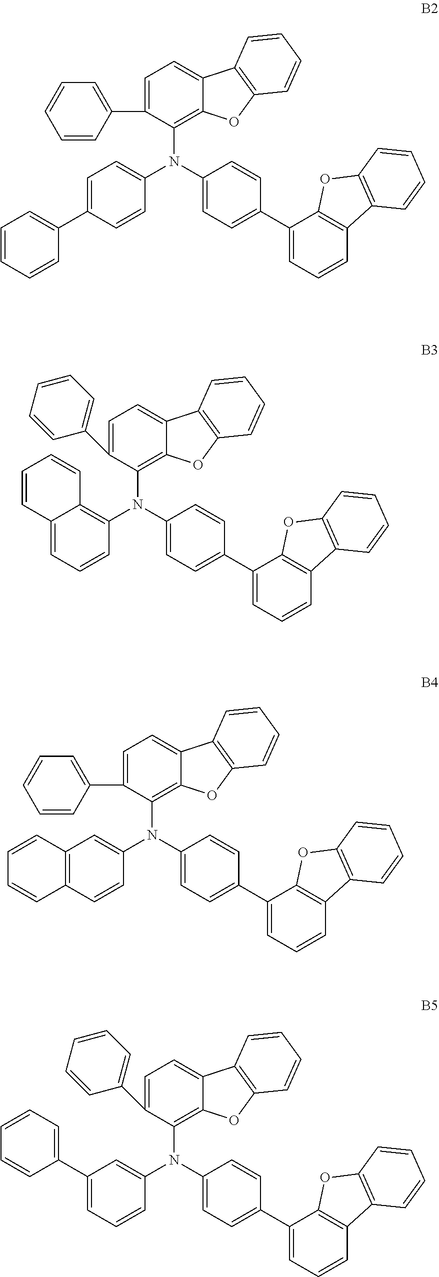 [SMILES: c1ccc(-c2ccc(N(c3ccc(-c4cccc5c4oc4ccccc45)cc3)c3c(-c4ccccc4)ccc4c3oc3ccccc34)cc2)cc1 ; c1ccc(-c2ccc3c(oc4ccccc43)c2N(c2ccc(-c3cccc4c3oc3ccccc34)cc2)c2cccc3ccccc23)cc1 ; c1ccc(-c2ccc3c(oc4ccccc43)c2N(c2ccc(-c3cccc4c3oc3ccccc34)cc2)c2ccc3ccccc3c2)cc1 ; c1ccc(-c2cccc(N(c3ccc(-c4cccc5c4oc4ccccc45)cc3)c3c(-c4ccccc4)ccc4c3oc3ccccc34)c2)cc1]

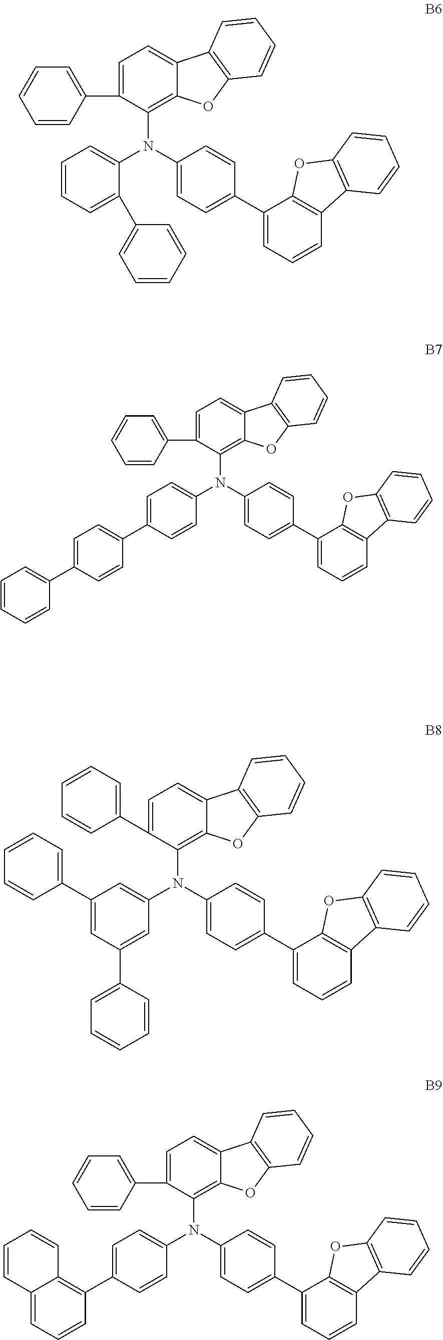 [SMILES: c1ccc(-c2ccccc2N(c2ccc(-c3cccc4c3oc3ccccc34)cc2)c2c(-c3ccccc3)ccc3c2oc2ccccc23)cc1 ; c1ccc(-c2ccc(-c3ccc(N(c4ccc(-c5cccc6c5oc5ccccc56)cc4)c4c(-c5ccccc5)ccc5c4oc4ccccc45)cc3)cc2)cc1 ; c1ccc(-c2cc(-c3ccccc3)cc(N(c3ccc(-c4cccc5c4oc4ccccc45)cc3)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc(-c3cccc4c3oc3ccccc34)cc2)cc1]

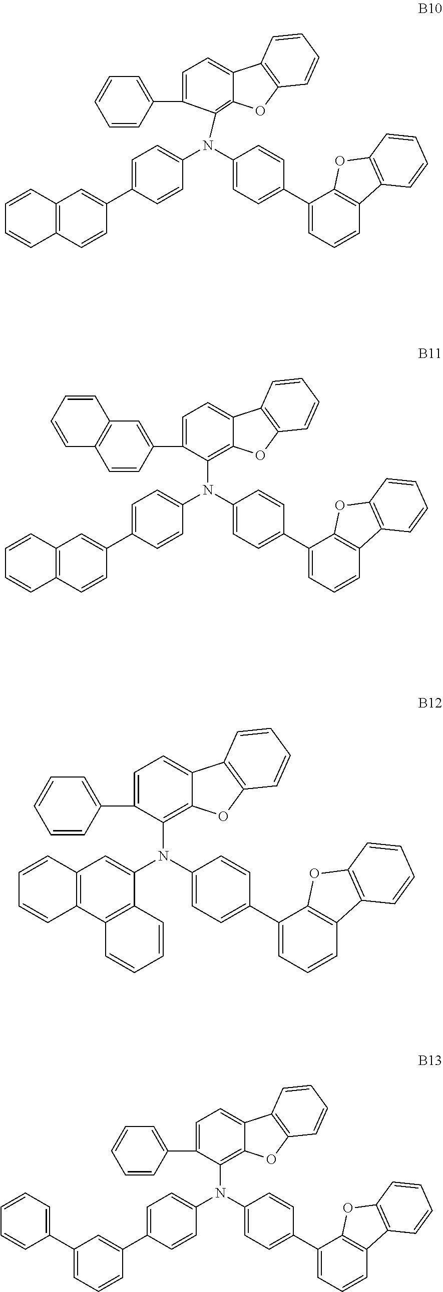 [SMILES: c1ccc(-c2ccc3c(oc4ccccc43)c2N(c2ccc(-c3ccc4ccccc4c3)cc2)c2ccc(-c3cccc4c3oc3ccccc34)cc2)cc1 ; c1ccc2cc(-c3ccc(N(c4ccc(-c5cccc6c5oc5ccccc56)cc4)c4c(-c5ccc6ccccc6c5)ccc5c4oc4ccccc45)cc3)ccc2c1 ; c1ccc(-c2ccc3c(oc4ccccc43)c2N(c2ccc(-c3cccc4c3oc3ccccc34)cc2)c2cc3ccccc3c3ccccc23)cc1 ; c1ccc(-c2cccc(-c3ccc(N(c4ccc(-c5cccc6c5oc5ccccc56)cc4)c4c(-c5ccccc5)ccc5c4oc4ccccc45)cc3)c2)cc1]

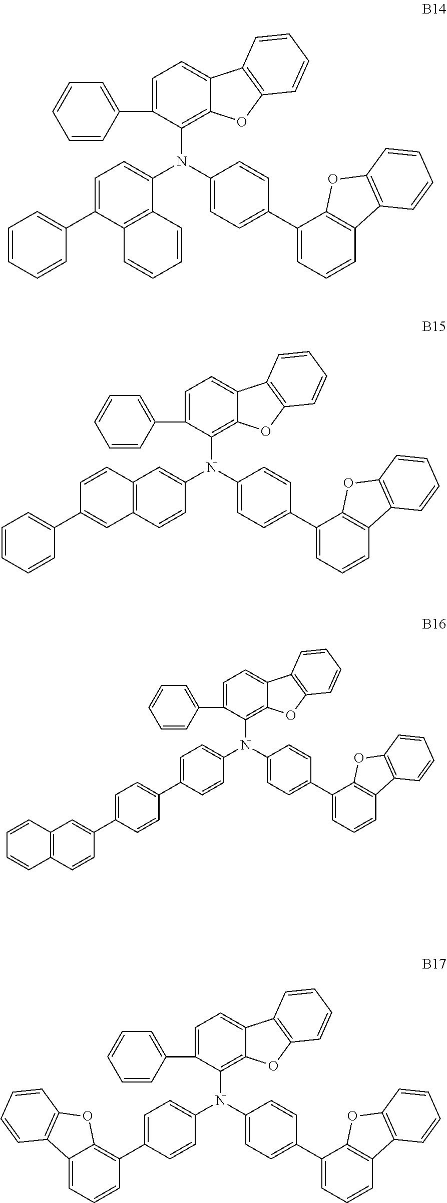 [SMILES: c1ccc(-c2ccc3c(oc4ccccc43)c2N(c2ccc(-c3cccc4c3oc3ccccc34)cc2)c2ccc(-c3ccccc3)c3ccccc23)cc1 ; c1ccc(-c2ccc3cc(N(c4ccc(-c5cccc6c5oc5ccccc56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4c3oc3ccccc34)cc2)cc1 ; c1ccc(-c2ccc3c(oc4ccccc43)c2N(c2ccc(-c3cccc4c3oc3ccccc34)cc2)c2ccc(-c3cccc4c3oc3ccccc34)cc2)cc1]

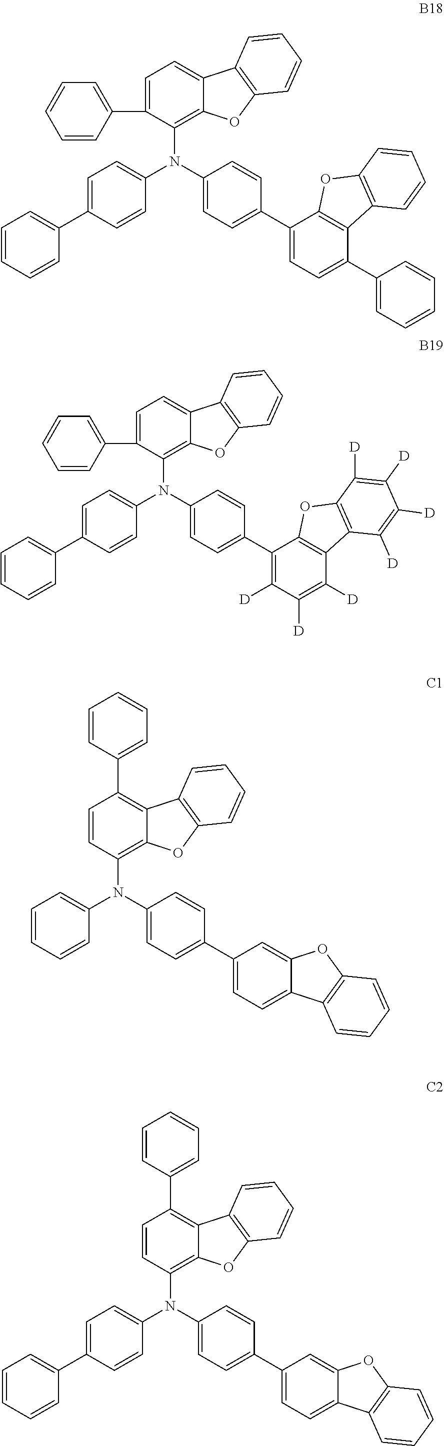 [SMILES: c1ccc(-c2ccc(N(c3ccc(-c4ccc(-c5ccccc5)c5c4oc4ccccc45)cc3)c3c(-c4ccccc4)ccc4c3oc3ccccc34)cc2)cc1 ; [2H]c1c([2H])c([2H])c2c(oc3c(-c4ccc(N(c5ccc(-c6ccccc6)cc5)c5c(-c6ccccc6)ccc6c5oc5ccccc56)cc4)c([2H])c([2H])c([2H])c32)c1[2H] ; c1ccc(-c2ccc(N(c3ccccc3)c3ccc(-c4ccc5c(c4)oc4ccccc45)cc3)c3oc4ccccc4c23)cc1 ; c1ccc(-c2ccc(N(c3ccc(-c4ccc5c(c4)oc4ccccc45)cc3)c3ccc(-c4ccccc4)c4c3oc3ccccc34)cc2)cc1]

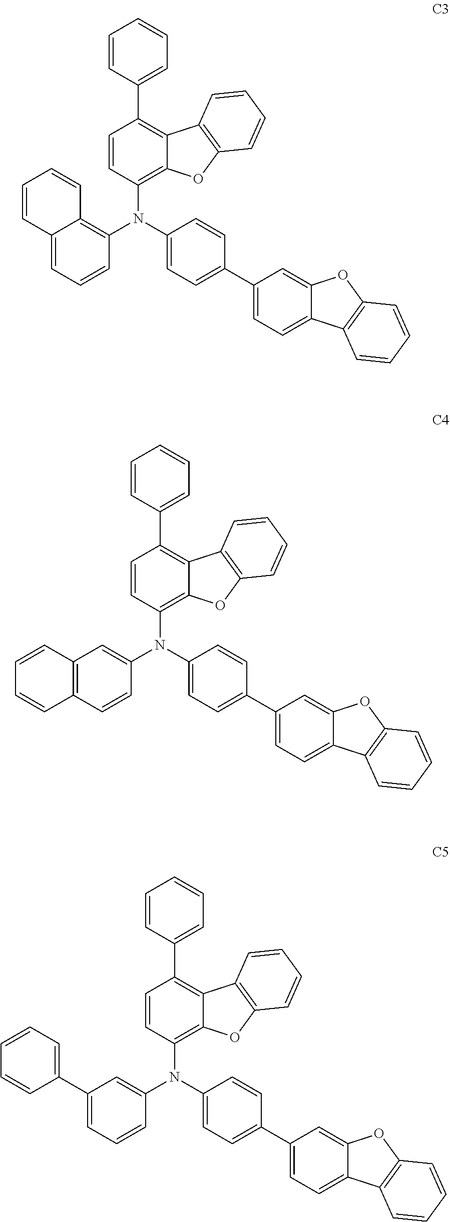 [SMILES: c1ccc(-c2ccc(N(c3ccc(-c4ccc5c(c4)oc4ccccc45)cc3)c3cccc4ccccc34)c3oc4ccccc4c23)cc1 ; c1ccc(-c2ccc(N(c3ccc(-c4ccc5c(c4)oc4ccccc45)cc3)c3ccc4ccccc4c3)c3oc4ccccc4c23)cc1 ; c1ccc(-c2cccc(N(c3ccc(-c4ccc5c(c4)oc4ccccc45)cc3)c3ccc(-c4ccccc4)c4c3oc3ccccc34)c2)cc1]

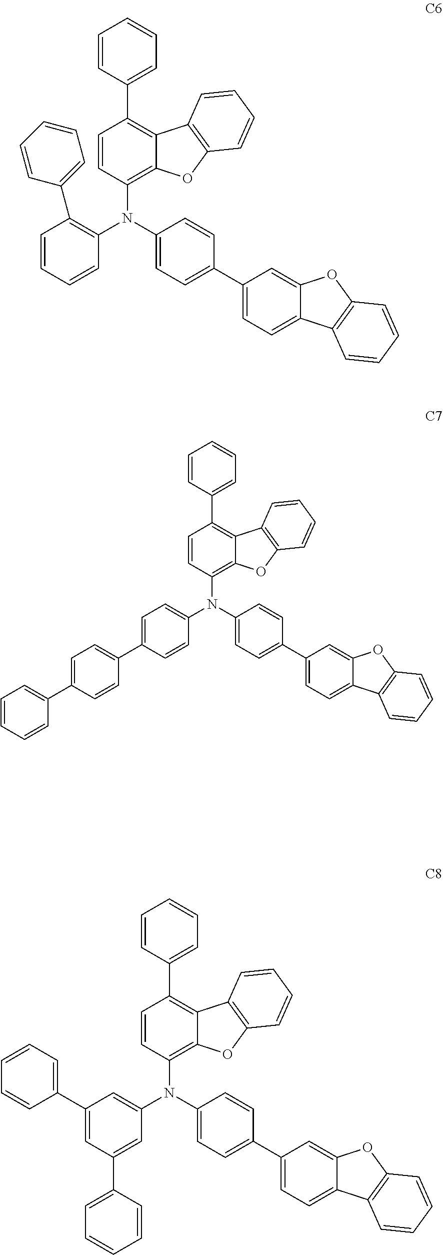 [SMILES: c1ccc(-c2ccccc2N(c2ccc(-c3ccc4c(c3)oc3ccccc34)cc2)c2ccc(-c3ccccc3)c3c2oc2ccccc23)cc1 ; c1ccc(-c2ccc(-c3ccc(N(c4ccc(-c5ccc6c(c5)oc5ccccc56)cc4)c4ccc(-c5ccccc5)c5c4oc4ccccc45)cc3)cc2)cc1 ; c1ccc(-c2cc(-c3ccccc3)cc(N(c3ccc(-c4ccc5c(c4)oc4ccccc45)cc3)c3ccc(-c4ccccc4)c4c3oc3ccccc34)c2)cc1]

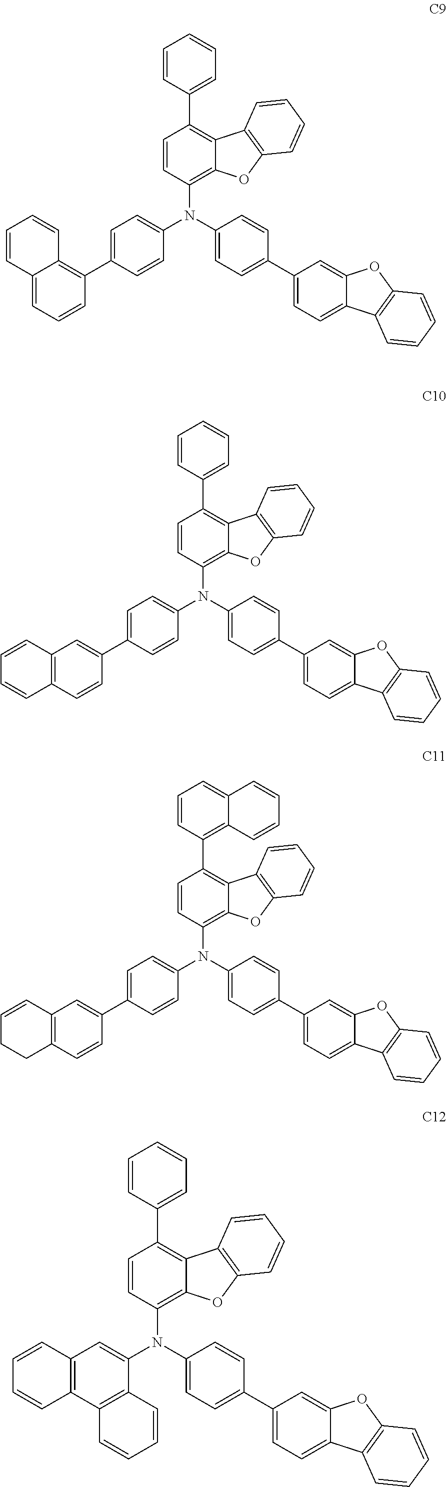 [SMILES: c1ccc(-c2ccc(N(c3ccc(-c4ccc5c(c4)oc4ccccc45)cc3)c3ccc(-c4cccc5ccccc45)cc3)c3oc4ccccc4c23)cc1 ; c1ccc(-c2ccc(N(c3ccc(-c4ccc5ccccc5c4)cc3)c3ccc(-c4ccc5c(c4)oc4ccccc45)cc3)c3oc4ccccc4c23)cc1 ; C1=Cc2cc(-c3ccc(N(c4ccc(-c5ccc6c(c5)oc5ccccc56)cc4)c4ccc(-c5cccc6ccccc56)c5c4oc4ccccc45)cc3)ccc2CC1 ; c1ccc(-c2ccc(N(c3ccc(-c4ccc5c(c4)oc4ccccc45)cc3)c3cc4ccccc4c4ccccc34)c3oc4ccccc4c23)cc1]

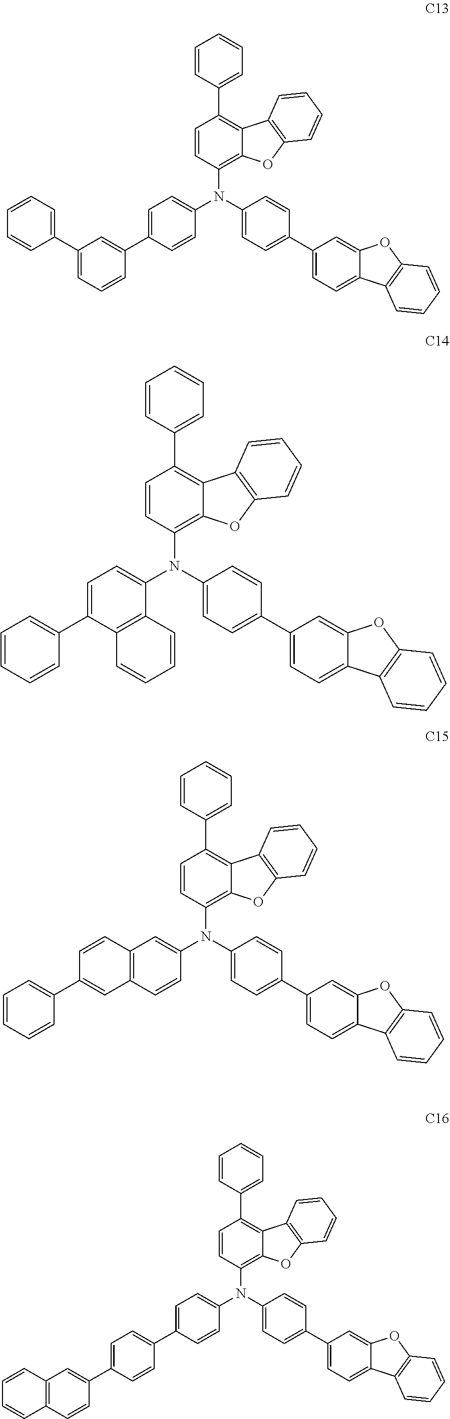 [SMILES: c1ccc(-c2cccc(-c3ccc(N(c4ccc(-c5ccc6c(c5)oc5ccccc56)cc4)c4ccc(-c5ccccc5)c5c4oc4ccccc45)cc3)c2)cc1 ; c1ccc(-c2ccc(N(c3ccc(-c4ccc5c(c4)oc4ccccc45)cc3)c3ccc(-c4ccccc4)c4c3oc3ccccc34)c3ccccc23)cc1 ; c1ccc(-c2ccc3cc(N(c4ccc(-c5ccc6c(c5)oc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4ccc5c(c4)oc4ccccc45)cc3)c3oc4ccccc4c23)cc1]

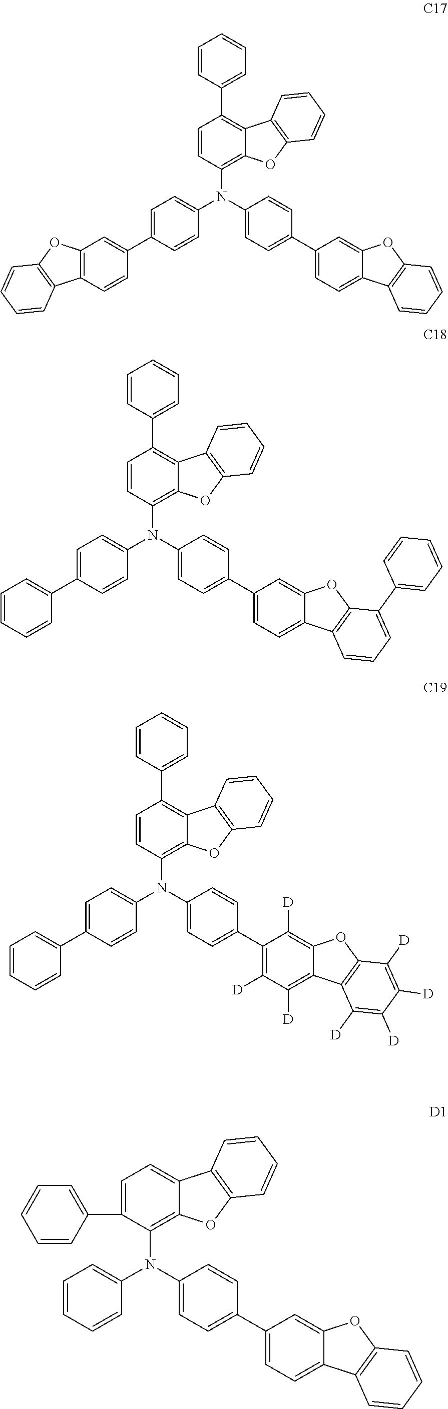 [SMILES: c1ccc(-c2ccc(N(c3ccc(-c4ccc5c(c4)oc4ccccc45)cc3)c3ccc(-c4ccc5c(c4)oc4ccccc45)cc3)c3oc4ccccc4c23)cc1 ; c1ccc(-c2ccc(N(c3ccc(-c4ccc5c(c4)oc4c(-c6ccccc6)cccc45)cc3)c3ccc(-c4ccccc4)c4c3oc3ccccc34)cc2)cc1 ; [2H]c1c([2H])c([2H])c2c(oc3c([2H])c(-c4ccc(N(c5ccc(-c6ccccc6)cc5)c5ccc(-c6ccccc6)c6c5oc5ccccc56)cc4)c([2H])c([2H])c32)c1[2H] ; c1ccc(-c2ccc3c(oc4ccccc43)c2N(c2ccccc2)c2ccc(-c3ccc4c(c3)oc3ccccc34)cc2)cc1]

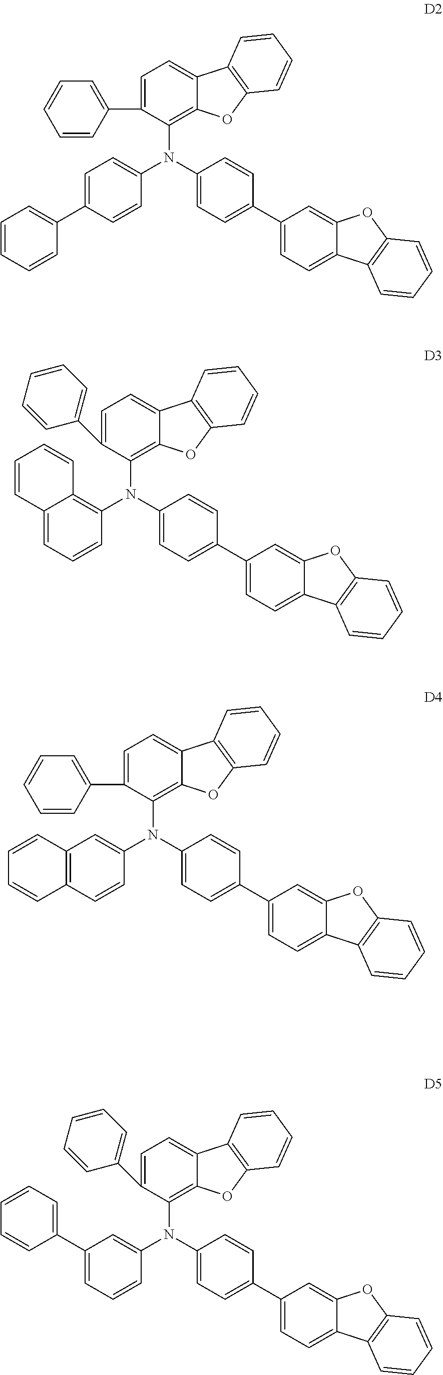 [SMILES: c1ccc(-c2ccc(N(c3ccc(-c4ccc5c(c4)oc4ccccc45)cc3)c3c(-c4ccccc4)ccc4c3oc3ccccc34)cc2)cc1 ; c1ccc(-c2ccc3c(oc4ccccc43)c2N(c2ccc(-c3ccc4c(c3)oc3ccccc34)cc2)c2cccc3ccccc23)cc1 ; c1ccc(-c2ccc3c(oc4ccccc43)c2N(c2ccc(-c3ccc4c(c3)oc3ccccc34)cc2)c2ccc3ccccc3c2)cc1 ; c1ccc(-c2cccc(N(c3ccc(-c4ccc5c(c4)oc4ccccc45)cc3)c3c(-c4ccccc4)ccc4c3oc3ccccc34)c2)cc1]

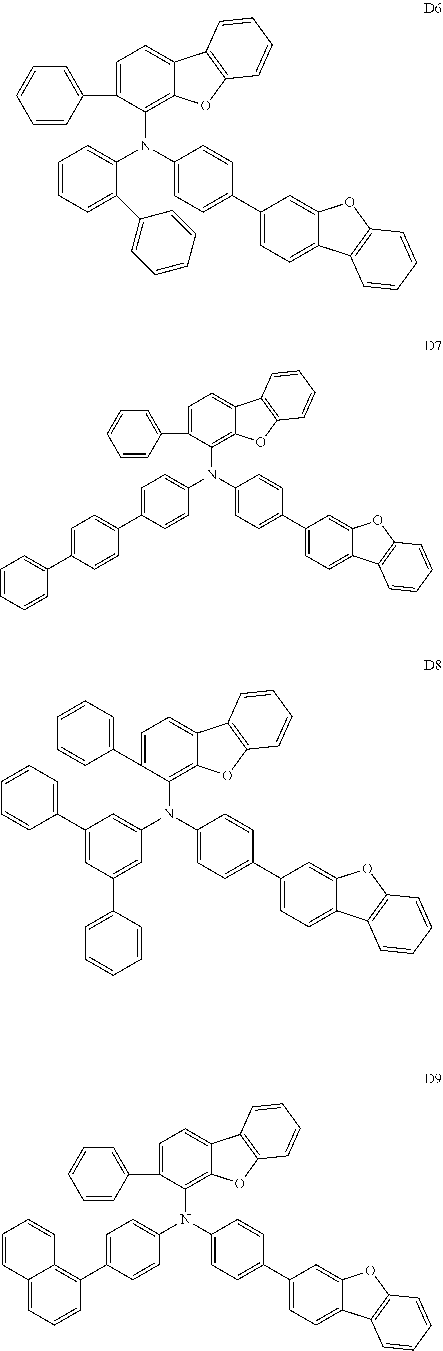 [SMILES: c1ccc(-c2ccccc2N(c2ccc(-c3ccc4c(c3)oc3ccccc34)cc2)c2c(-c3ccccc3)ccc3c2oc2ccccc23)cc1 ; c1ccc(-c2ccc(-c3ccc(N(c4ccc(-c5ccc6c(c5)oc5ccccc56)cc4)c4c(-c5ccccc5)ccc5c4oc4ccccc45)cc3)cc2)cc1 ; c1ccc(-c2cc(-c3ccccc3)cc(N(c3ccc(-c4ccc5c(c4)oc4ccccc45)cc3)c3c(-c4ccccc4)ccc4c3oc3ccccc34)c2)cc1 ; c1ccc(-c2ccc3c(oc4ccccc43)c2N(c2ccc(-c3ccc4c(c3)oc3ccccc34)cc2)c2ccc(-c3cccc4ccccc34)cc2)cc1]

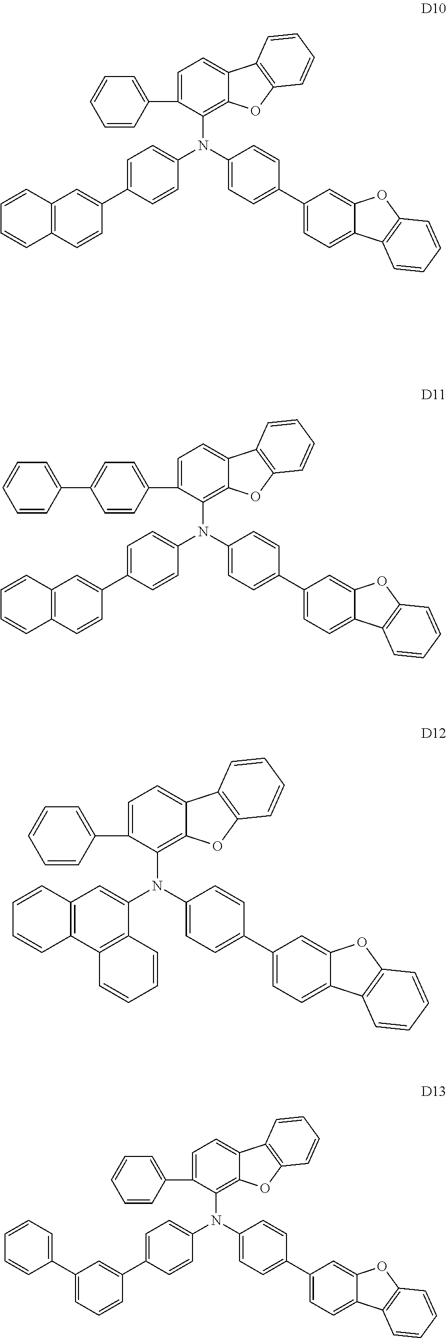 [SMILES: c1ccc(-c2ccc3c(oc4ccccc43)c2N(c2ccc(-c3ccc4ccccc4c3)cc2)c2ccc(-c3ccc4c(c3)oc3ccccc34)cc2)cc1 ; c1ccc(-c2ccc(-c3ccc4c(oc5ccccc54)c3N(c3ccc(-c4ccc5ccccc5c4)cc3)c3ccc(-c4ccc5c(c4)oc4ccccc45)cc3)cc2)cc1 ; c1ccc(-c2ccc3c(oc4ccccc43)c2N(c2ccc(-c3ccc4c(c3)oc3ccccc34)cc2)c2cc3ccccc3c3ccccc23)cc1 ; c1ccc(-c2cccc(-c3ccc(N(c4ccc(-c5ccc6c(c5)oc5ccccc56)cc4)c4c(-c5ccccc5)ccc5c4oc4ccccc45)cc3)c2)cc1]

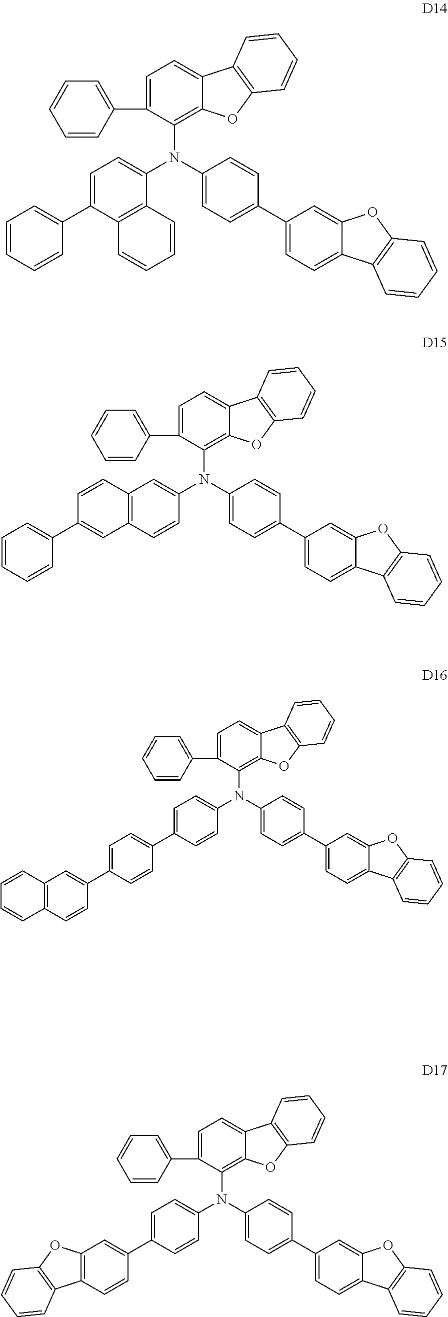 [SMILES: c1ccc(-c2ccc3c(oc4ccccc43)c2N(c2ccc(-c3ccc4c(c3)oc3ccccc34)cc2)c2ccc(-c3ccccc3)c3ccccc23)cc1 ; c1ccc(-c2ccc3cc(N(c4ccc(-c5ccc6c(c5)oc5ccccc56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3ccc4c(c3)oc3ccccc34)cc2)cc1 ; c1ccc(-c2ccc3c(oc4ccccc43)c2N(c2ccc(-c3ccc4c(c3)oc3ccccc34)cc2)c2ccc(-c3ccc4c(c3)oc3ccccc34)cc2)cc1]

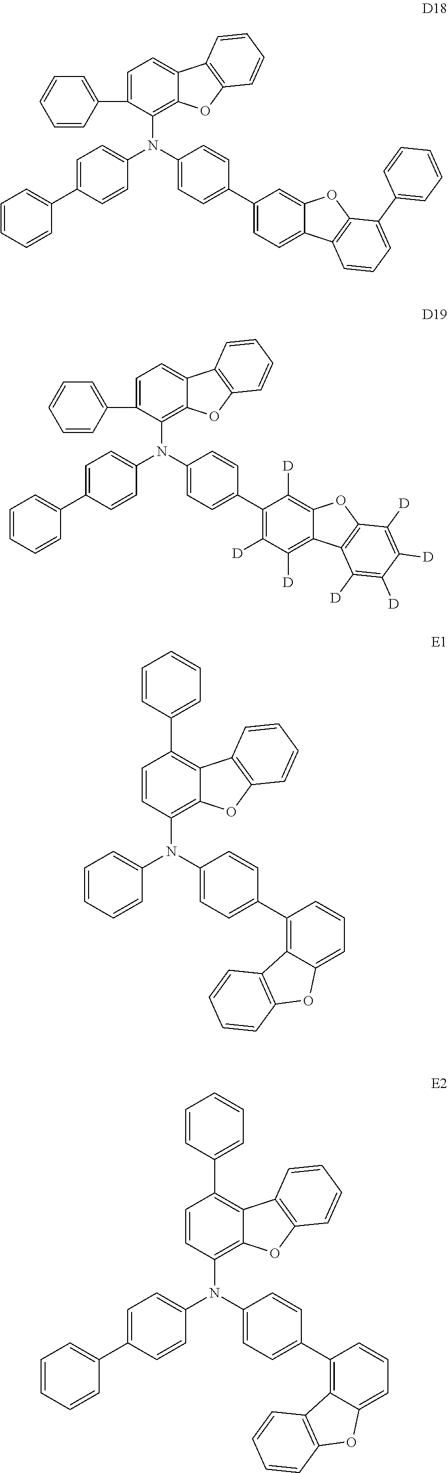 [SMILES: c1ccc(-c2ccc(N(c3ccc(-c4ccc5c(c4)oc4c(-c6ccccc6)cccc45)cc3)c3c(-c4ccccc4)ccc4c3oc3ccccc34)cc2)cc1 ; [2H]c1c([2H])c([2H])c2c(oc3c([2H])c(-c4ccc(N(c5ccc(-c6ccccc6)cc5)c5c(-c6ccccc6)ccc6c5oc5ccccc56)cc4)c([2H])c([2H])c32)c1[2H] ; c1ccc(-c2ccc(N(c3ccccc3)c3ccc(-c4cccc5oc6ccccc6c45)cc3)c3oc4ccccc4c23)cc1 ; c1ccc(-c2ccc(N(c3ccc(-c4cccc5oc6ccccc6c45)cc3)c3ccc(-c4ccccc4)c4c3oc3ccccc34)cc2)cc1]

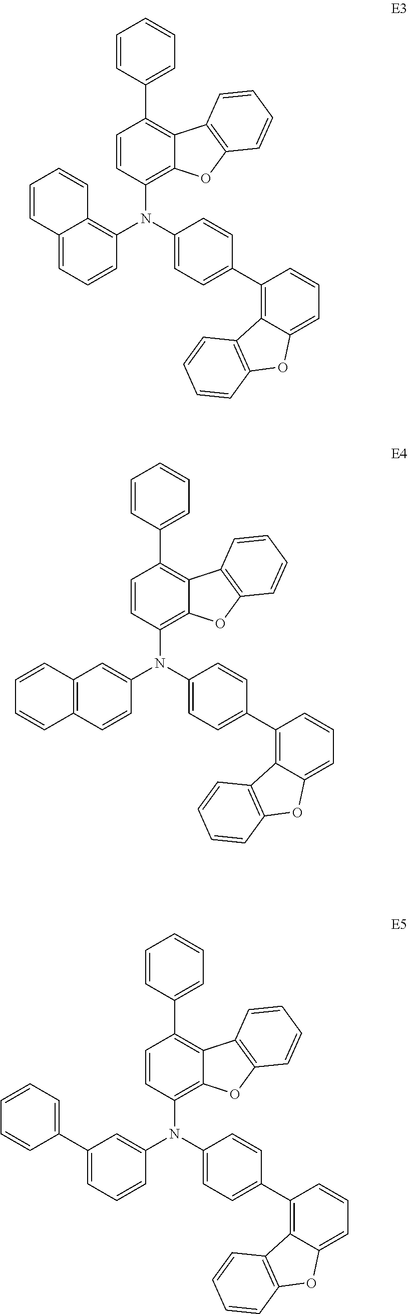 [SMILES: c1ccc(-c2ccc(N(c3ccc(-c4cccc5oc6ccccc6c45)cc3)c3cccc4ccccc34)c3oc4ccccc4c23)cc1 ; c1ccc(-c2ccc(N(c3ccc(-c4cccc5oc6ccccc6c45)cc3)c3ccc4ccccc4c3)c3oc4ccccc4c23)cc1 ; c1ccc(-c2cccc(N(c3ccc(-c4cccc5oc6ccccc6c45)cc3)c3ccc(-c4ccccc4)c4c3oc3ccccc34)c2)cc1]

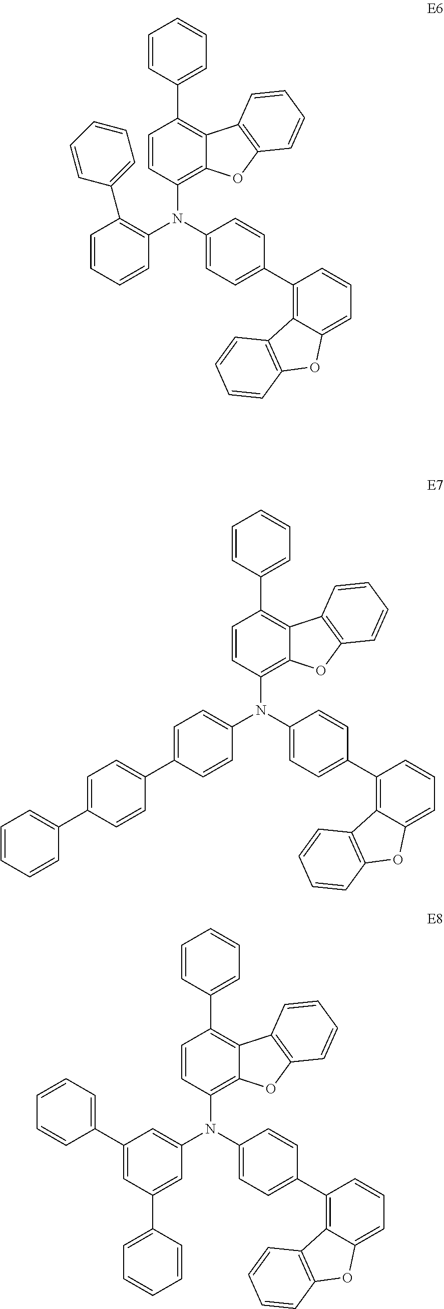 [SMILES: c1ccc(-c2ccccc2N(c2ccc(-c3cccc4oc5ccccc5c34)cc2)c2ccc(-c3ccccc3)c3c2oc2ccccc23)cc1 ; c1ccc(-c2ccc(-c3ccc(N(c4ccc(-c5cccc6oc7ccccc7c56)cc4)c4ccc(-c5ccccc5)c5c4oc4ccccc45)cc3)cc2)cc1 ; c1ccc(-c2cc(-c3ccccc3)cc(N(c3ccc(-c4cccc5oc6ccccc6c45)cc3)c3ccc(-c4ccccc4)c4c3oc3ccccc34)c2)cc1]

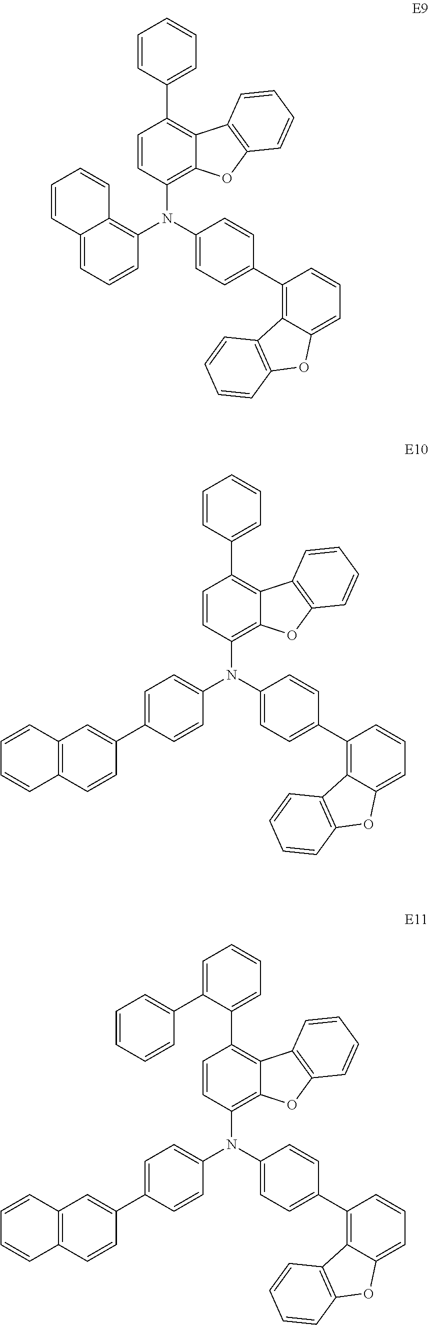 [SMILES: c1ccc(-c2ccc(N(c3ccc(-c4cccc5oc6ccccc6c45)cc3)c3cccc4ccccc34)c3oc4ccccc4c23)cc1 ; c1ccc(-c2ccc(N(c3ccc(-c4ccc5ccccc5c4)cc3)c3ccc(-c4cccc5oc6ccccc6c45)cc3)c3oc4ccccc4c23)cc1 ; c1ccc(-c2ccccc2-c2ccc(N(c3ccc(-c4ccc5ccccc5c4)cc3)c3ccc(-c4cccc5oc6ccccc6c45)cc3)c3oc4ccccc4c23)cc1]

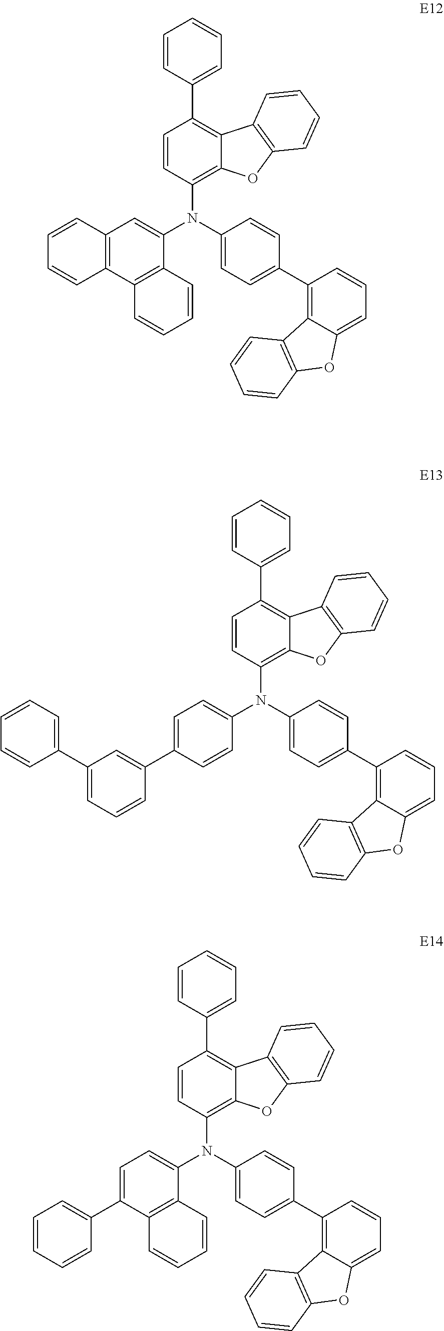 [SMILES: c1ccc(-c2ccc(N(c3ccc(-c4cccc5oc6ccccc6c45)cc3)c3cc4ccccc4c4ccccc34)c3oc4ccccc4c23)cc1 ; c1ccc(-c2cccc(-c3ccc(N(c4ccc(-c5cccc6oc7ccccc7c56)cc4)c4ccc(-c5ccccc5)c5c4oc4ccccc45)cc3)c2)cc1 ; c1ccc(-c2ccc(N(c3ccc(-c4cccc5oc6ccccc6c45)cc3)c3ccc(-c4ccccc4)c4c3oc3ccccc34)c3ccccc23)cc1]

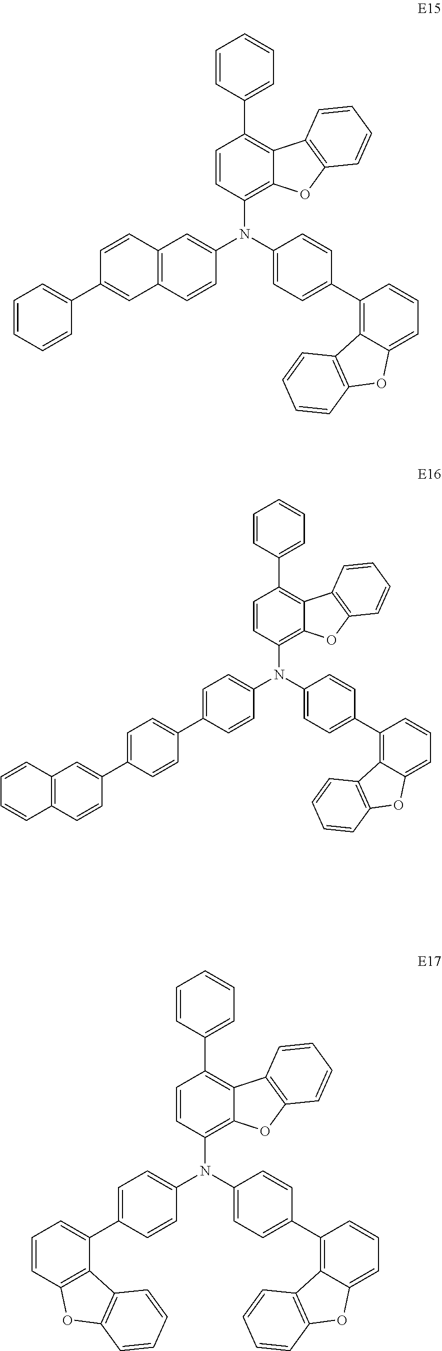 [SMILES: c1ccc(-c2ccc3cc(N(c4ccc(-c5cccc6oc7ccccc7c56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4cccc5oc6ccccc6c45)cc3)c3oc4ccccc4c23)cc1 ; c1ccc(-c2ccc(N(c3ccc(-c4cccc5oc6ccccc6c45)cc3)c3ccc(-c4cccc5oc6ccccc6c45)cc3)c3oc4ccccc4c23)cc1]

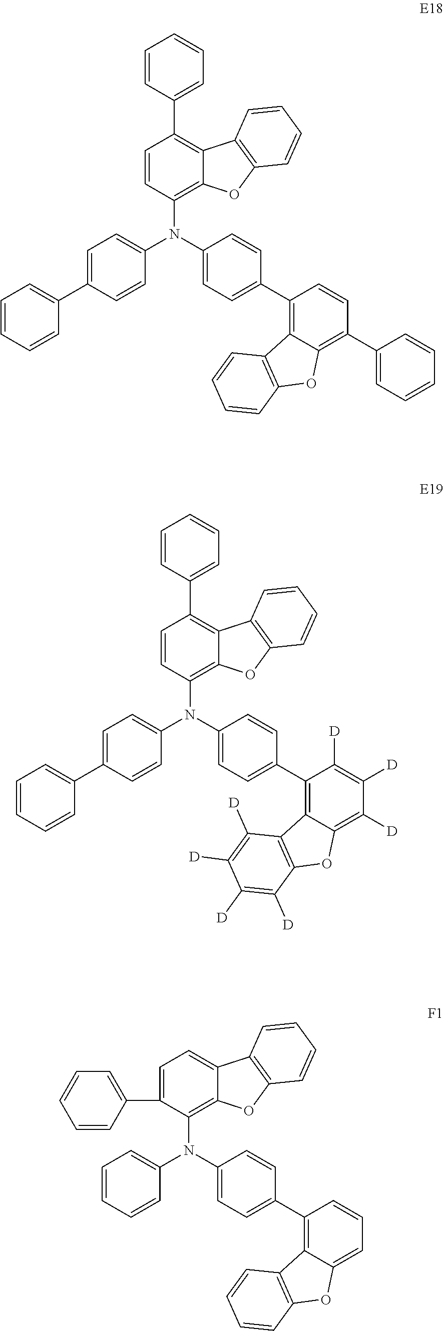 [SMILES: c1ccc(-c2ccc(N(c3ccc(-c4ccc(-c5ccccc5)c5oc6ccccc6c45)cc3)c3ccc(-c4ccccc4)c4c3oc3ccccc34)cc2)cc1 ; [2H]c1c([2H])c([2H])c2c(oc3c([2H])c([2H])c([2H])c(-c4ccc(N(c5ccc(-c6ccccc6)cc5)c5ccc(-c6ccccc6)c6c5oc5ccccc56)cc4)c32)c1[2H] ; c1ccc(-c2ccc3c(oc4ccccc43)c2N(c2ccccc2)c2ccc(-c3cccc4oc5ccccc5c34)cc2)cc1]

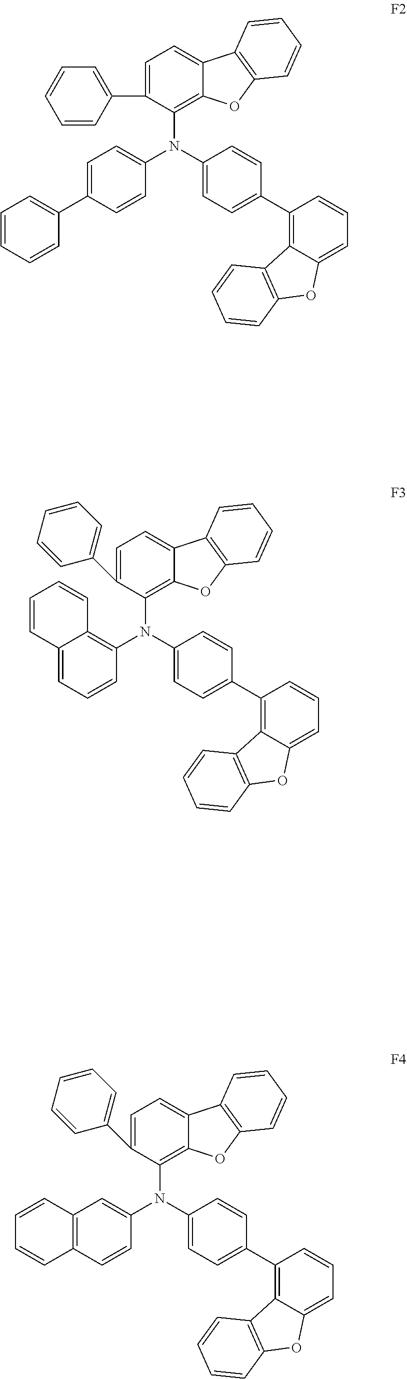 [SMILES: c1ccc(-c2ccc(N(c3ccc(-c4cccc5oc6ccccc6c45)cc3)c3c(-c4ccccc4)ccc4c3oc3ccccc34)cc2)cc1 ; c1ccc(-c2ccc3c(oc4ccccc43)c2N(c2ccc(-c3cccc4oc5ccccc5c34)cc2)c2cccc3ccccc23)cc1 ; c1ccc(-c2ccc3c(oc4ccccc43)c2N(c2ccc(-c3cccc4oc5ccccc5c34)cc2)c2ccc3ccccc3c2)cc1]

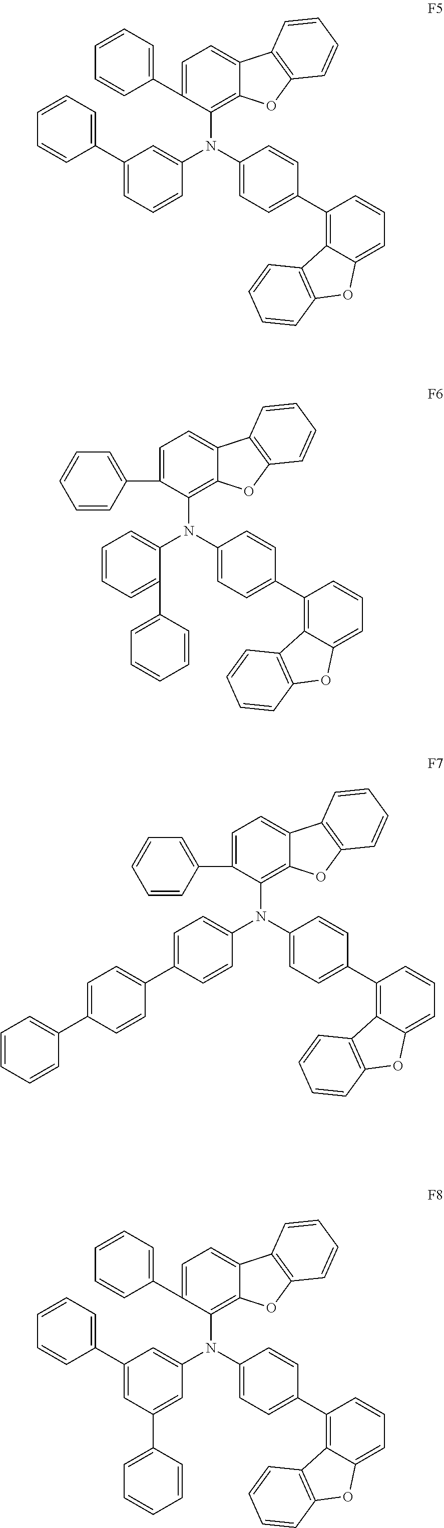 [SMILES: c1ccc(-c2cccc(N(c3ccc(-c4cccc5oc6ccccc6c45)cc3)c3c(-c4ccccc4)ccc4c3oc3ccccc34)c2)cc1 ; c1ccc(-c2ccccc2N(c2ccc(-c3cccc4oc5ccccc5c34)cc2)c2c(-c3ccccc3)ccc3c2oc2ccccc23)cc1 ; c1ccc(-c2ccc(-c3ccc(N(c4ccc(-c5cccc6oc7ccccc7c56)cc4)c4c(-c5ccccc5)ccc5c4oc4ccccc45)cc3)cc2)cc1 ; c1ccc(-c2cc(-c3ccccc3)cc(N(c3ccc(-c4cccc5oc6ccccc6c45)cc3)c3c(-c4ccccc4)ccc4c3oc3ccccc34)c2)cc1]

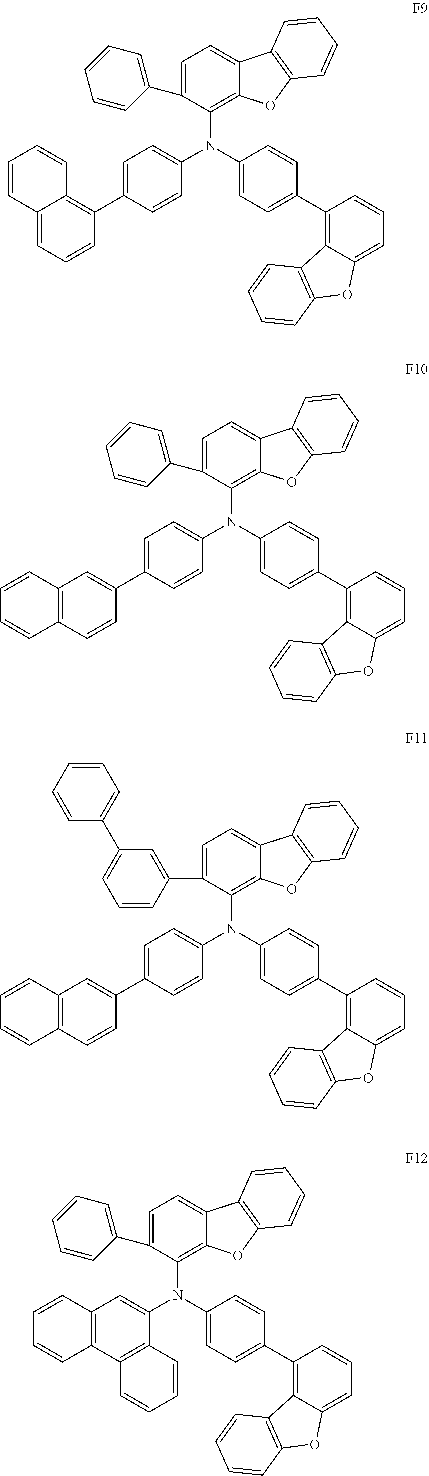 [SMILES: c1ccc(-c2ccc3c(oc4ccccc43)c2N(c2ccc(-c3cccc4ccccc34)cc2)c2ccc(-c3cccc4oc5ccccc5c34)cc2)cc1 ; c1ccc(-c2ccc3c(oc4ccccc43)c2N(c2ccc(-c3ccc4ccccc4c3)cc2)c2ccc(-c3cccc4oc5ccccc5c34)cc2)cc1 ; c1ccc(-c2cccc(-c3ccc4c(oc5ccccc54)c3N(c3ccc(-c4ccc5ccccc5c4)cc3)c3ccc(-c4cccc5oc6ccccc6c45)cc3)c2)cc1 ; c1ccc(-c2ccc3c(oc4ccccc43)c2N(c2ccc(-c3cccc4oc5ccccc5c34)cc2)c2cc3ccccc3c3ccccc23)cc1]

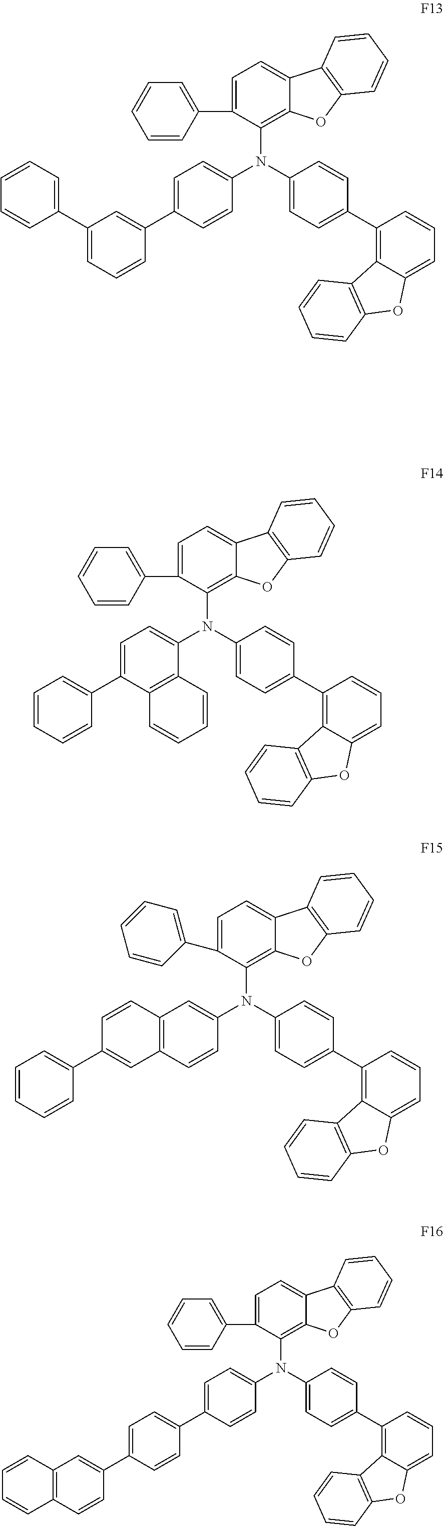 [SMILES: c1ccc(-c2cccc(-c3ccc(N(c4ccc(-c5cccc6oc7ccccc7c56)cc4)c4c(-c5ccccc5)ccc5c4oc4ccccc45)cc3)c2)cc1 ; c1ccc(-c2ccc3c(oc4ccccc43)c2N(c2ccc(-c3cccc4oc5ccccc5c34)cc2)c2ccc(-c3ccccc3)c3ccccc23)cc1 ; c1ccc(-c2ccc3cc(N(c4ccc(-c5cccc6oc7ccccc7c56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4oc5ccccc5c34)cc2)cc1]

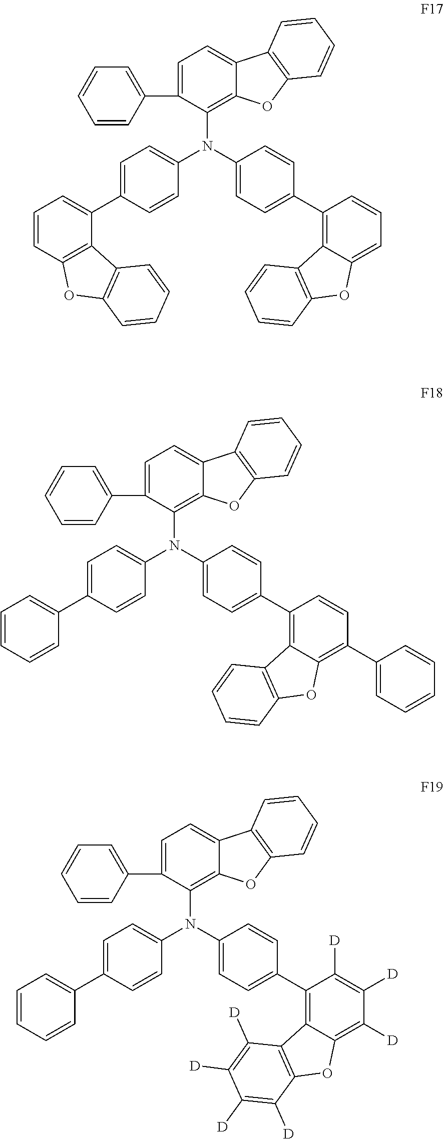 [SMILES: c1ccc(-c2ccc3c(oc4ccccc43)c2N(c2ccc(-c3cccc4oc5ccccc5c34)cc2)c2ccc(-c3cccc4oc5ccccc5c34)cc2)cc1 ; c1ccc(-c2ccc(N(c3ccc(-c4ccc(-c5ccccc5)c5oc6ccccc6c45)cc3)c3c(-c4ccccc4)ccc4c3oc3ccccc34)cc2)cc1 ; [2H]c1c([2H])c([2H])c2c(oc3c([2H])c([2H])c([2H])c(-c4ccc(N(c5ccc(-c6ccccc6)cc5)c5c(-c6ccccc6)ccc6c5oc5ccccc56)cc4)c32)c1[2H]]

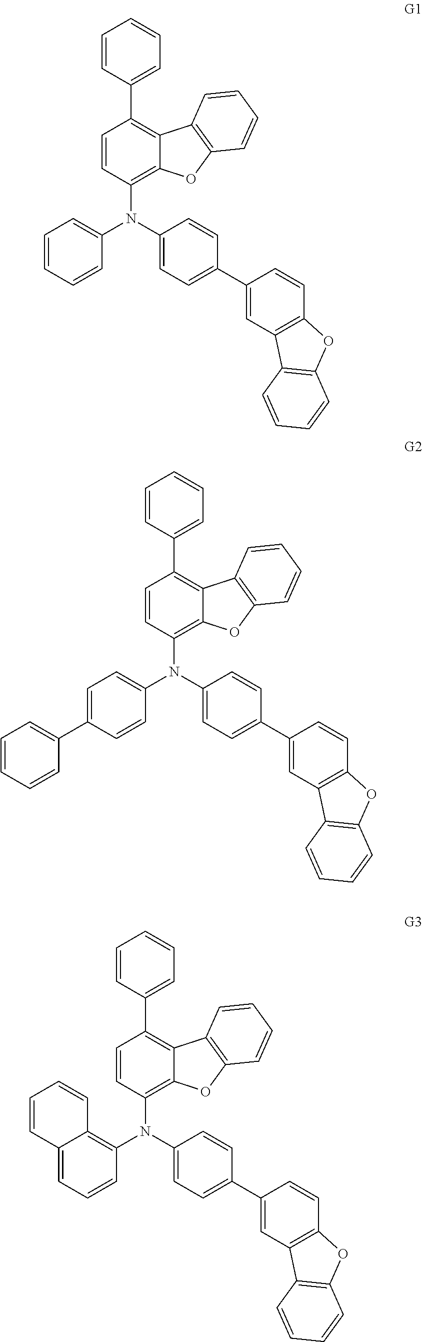 [SMILES: c1ccc(-c2ccc(N(c3ccccc3)c3ccc(-c4ccc5oc6ccccc6c5c4)cc3)c3oc4ccccc4c23)cc1 ; c1ccc(-c2ccc(N(c3ccc(-c4ccc5oc6ccccc6c5c4)cc3)c3ccc(-c4ccccc4)c4c3oc3ccccc34)cc2)cc1 ; c1ccc(-c2ccc(N(c3ccc(-c4ccc5oc6ccccc6c5c4)cc3)c3cccc4ccccc34)c3oc4ccccc4c23)cc1]

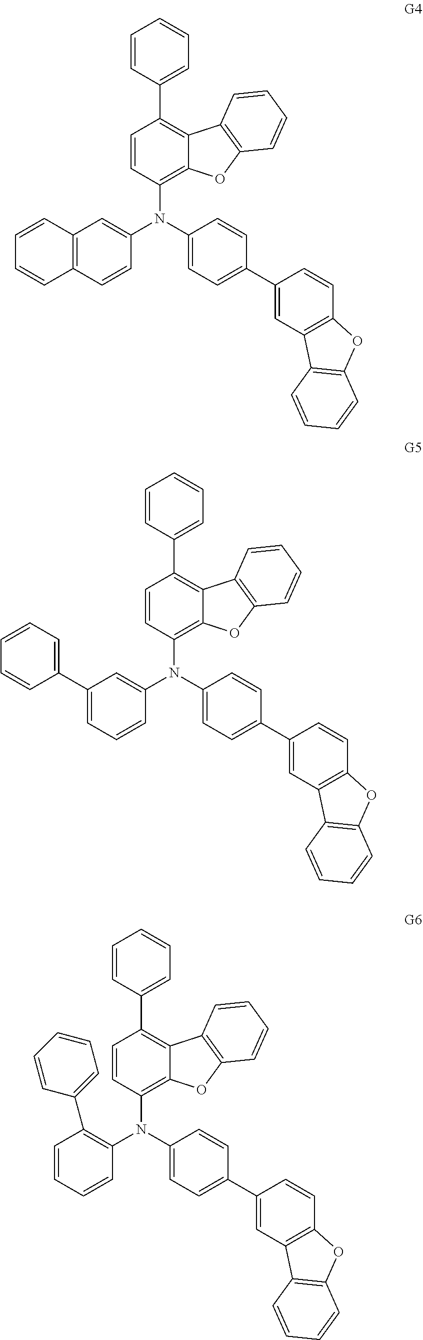 [SMILES: c1ccc(-c2ccc(N(c3ccc(-c4ccc5oc6ccccc6c5c4)cc3)c3ccc4ccccc4c3)c3oc4ccccc4c23)cc1 ; c1ccc(-c2cccc(N(c3ccc(-c4ccc5oc6ccccc6c5c4)cc3)c3ccc(-c4ccccc4)c4c3oc3ccccc34)c2)cc1 ; c1ccc(-c2ccccc2N(c2ccc(-c3ccc4oc5ccccc5c4c3)cc2)c2ccc(-c3ccccc3)c3c2oc2ccccc23)cc1]

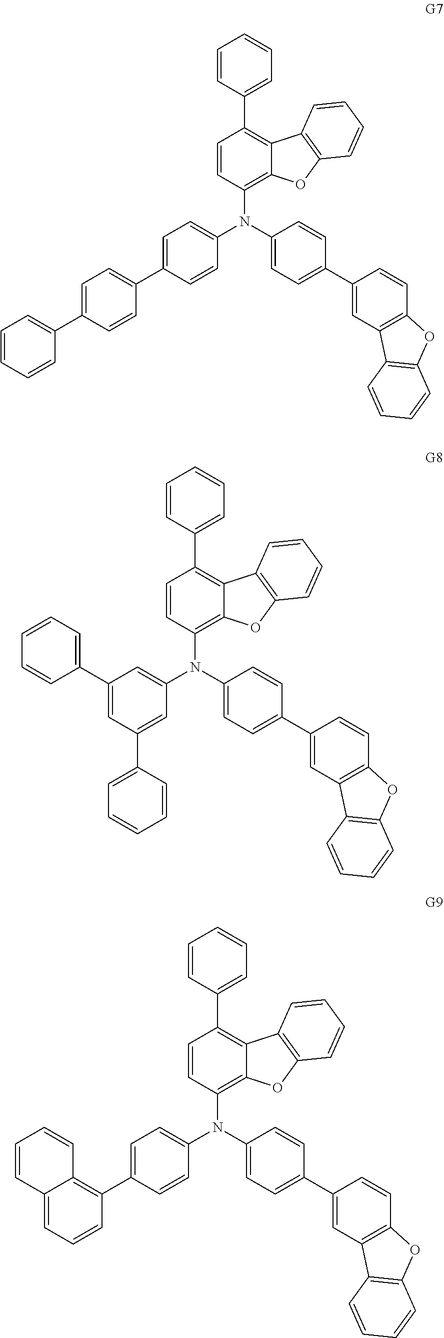 [SMILES: c1ccc(-c2ccc(-c3ccc(N(c4ccc(-c5ccc6oc7ccccc7c6c5)cc4)c4ccc(-c5ccccc5)c5c4oc4ccccc45)cc3)cc2)cc1 ; c1ccc(-c2cc(-c3ccccc3)cc(N(c3ccc(-c4ccc5oc6ccccc6c5c4)cc3)c3ccc(-c4ccccc4)c4c3oc3ccccc34)c2)cc1 ; c1ccc(-c2ccc(N(c3ccc(-c4ccc5oc6ccccc6c5c4)cc3)c3ccc(-c4cccc5ccccc45)cc3)c3oc4ccccc4c23)cc1]

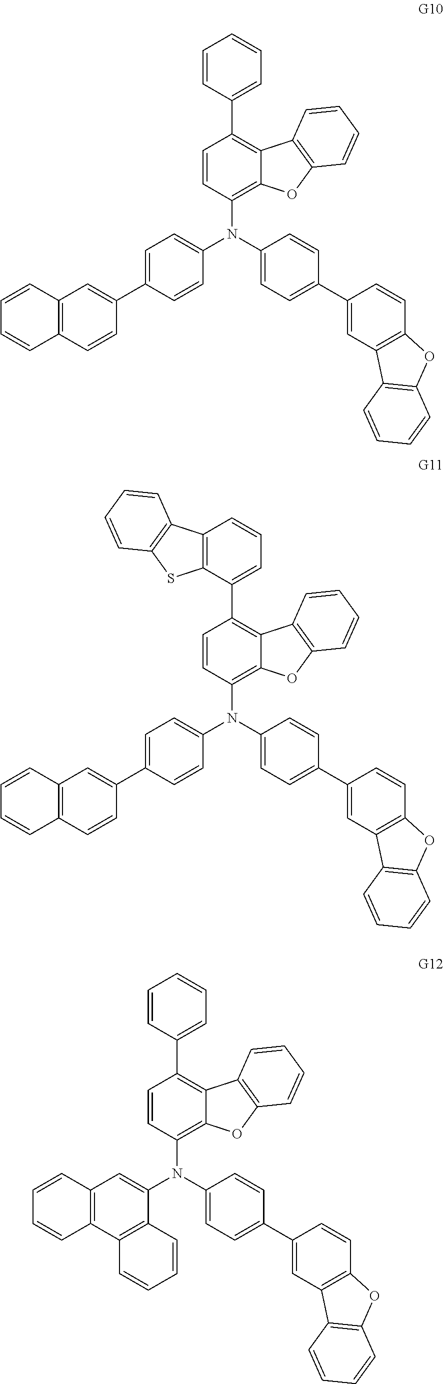 [SMILES: c1ccc(-c2ccc(N(c3ccc(-c4ccc5ccccc5c4)cc3)c3ccc(-c4ccc5oc6ccccc6c5c4)cc3)c3oc4ccccc4c23)cc1 ; c1ccc2cc(-c3ccc(N(c4ccc(-c5ccc6oc7ccccc7c6c5)cc4)c4ccc(-c5cccc6c5sc5ccccc56)c5c4oc4ccccc45)cc3)ccc2c1 ; c1ccc(-c2ccc(N(c3ccc(-c4ccc5oc6ccccc6c5c4)cc3)c3cc4ccccc4c4ccccc34)c3oc4ccccc4c23)cc1]

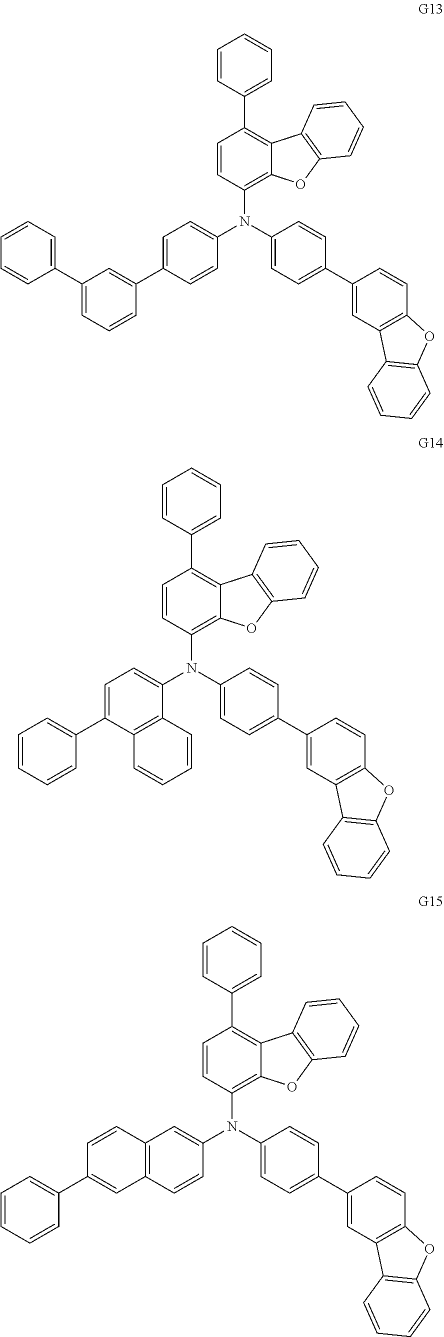 [SMILES: c1ccc(-c2cccc(-c3ccc(N(c4ccc(-c5ccc6oc7ccccc7c6c5)cc4)c4ccc(-c5ccccc5)c5c4oc4ccccc45)cc3)c2)cc1 ; c1ccc(-c2ccc(N(c3ccc(-c4ccc5oc6ccccc6c5c4)cc3)c3ccc(-c4ccccc4)c4c3oc3ccccc34)c3ccccc23)cc1 ; c1ccc(-c2ccc3cc(N(c4ccc(-c5ccc6oc7ccccc7c6c5)cc4)c4ccc(-c5ccccc5)c5c4oc4ccccc45)ccc3c2)cc1]

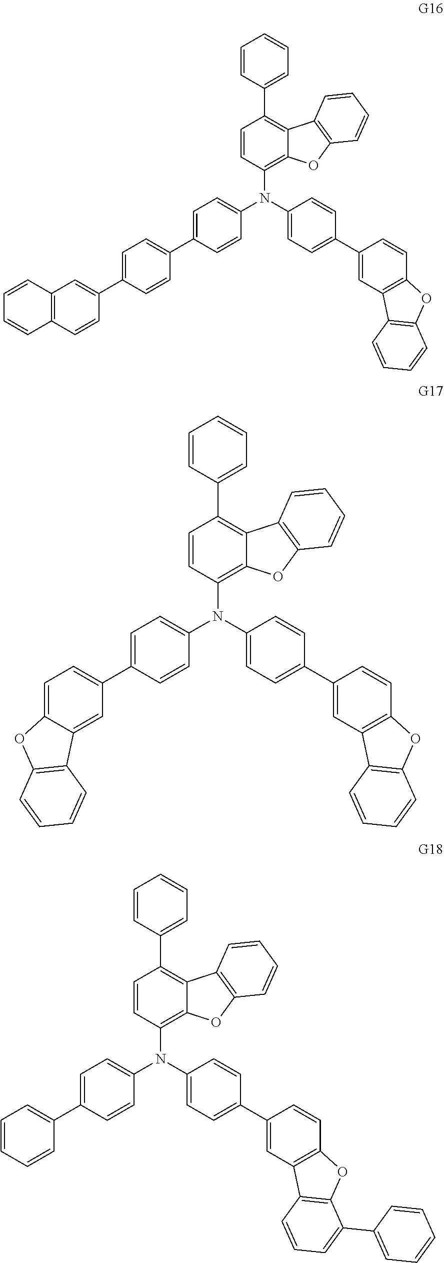 [SMILES: c1ccc(-c2ccc(N(c3ccc(-c4ccc(-c5ccc6ccccc6c5)cc4)cc3)c3ccc(-c4ccc5oc6ccccc6c5c4)cc3)c3oc4ccccc4c23)cc1 ; c1ccc(-c2ccc(N(c3ccc(-c4ccc5oc6ccccc6c5c4)cc3)c3ccc(-c4ccc5oc6ccccc6c5c4)cc3)c3oc4ccccc4c23)cc1 ; c1ccc(-c2ccc(N(c3ccc(-c4ccc5oc6c(-c7ccccc7)cccc6c5c4)cc3)c3ccc(-c4ccccc4)c4c3oc3ccccc34)cc2)cc1]

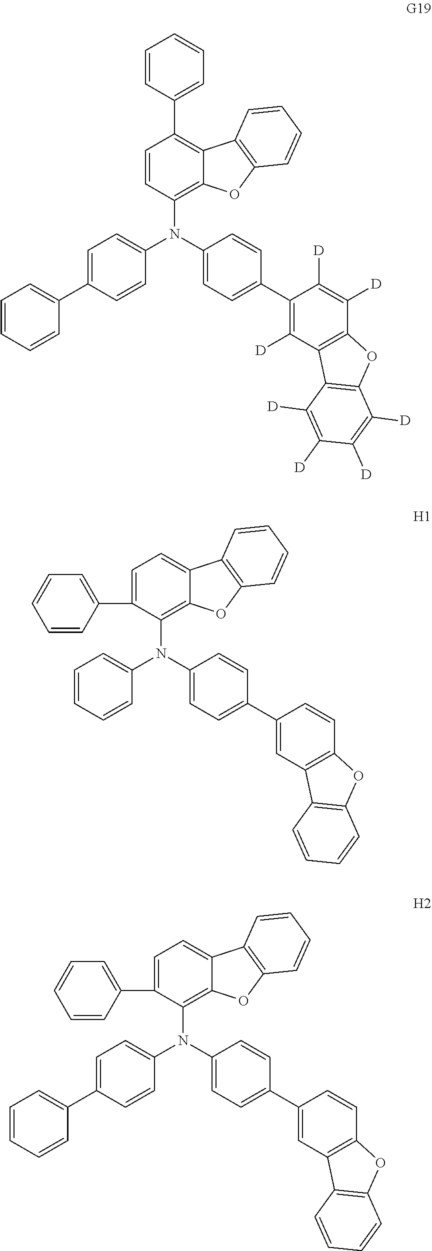 [SMILES: [2H]c1c([2H])c([2H])c2c(oc3c([2H])c([2H])c(-c4ccc(N(c5ccc(-c6ccccc6)cc5)c5ccc(-c6ccccc6)c6c5oc5ccccc56)cc4)c([2H])c32)c1[2H] ; c1ccc(-c2ccc3c(oc4ccccc43)c2N(c2ccccc2)c2ccc(-c3ccc4oc5ccccc5c4c3)cc2)cc1 ; c1ccc(-c2ccc(N(c3ccc(-c4ccc5oc6ccccc6c5c4)cc3)c3c(-c4ccccc4)ccc4c3oc3ccccc34)cc2)cc1]

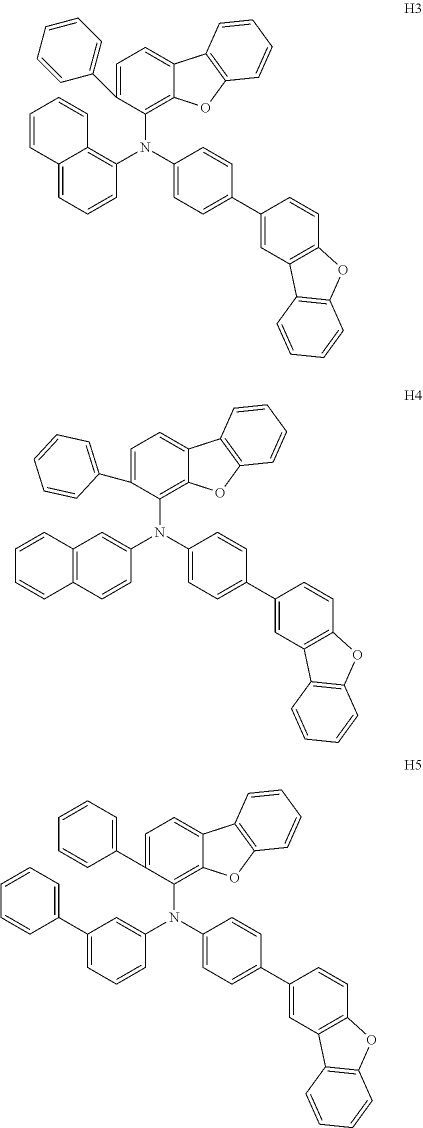 [SMILES: c1ccc(-c2ccc3c(oc4ccccc43)c2N(c2ccc(-c3ccc4oc5ccccc5c4c3)cc2)c2cccc3ccccc23)cc1 ; c1ccc(-c2ccc3c(oc4ccccc43)c2N(c2ccc(-c3ccc4oc5ccccc5c4c3)cc2)c2ccc3ccccc3c2)cc1 ; c1ccc(-c2cccc(N(c3ccc(-c4ccc5oc6ccccc6c5c4)cc3)c3c(-c4ccccc4)ccc4c3oc3ccccc34)c2)cc1]

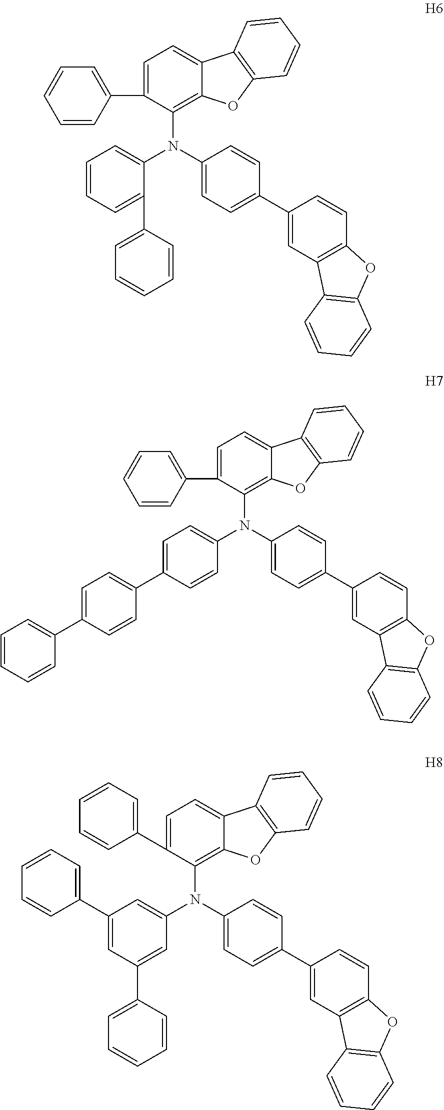 [SMILES: c1ccc(-c2ccccc2N(c2ccc(-c3ccc4oc5ccccc5c4c3)cc2)c2c(-c3ccccc3)ccc3c2oc2ccccc23)cc1 ; c1ccc(-c2ccc(-c3ccc(N(c4ccc(-c5ccc6oc7ccccc7c6c5)cc4)c4c(-c5ccccc5)ccc5c4oc4ccccc45)cc3)cc2)cc1 ; c1ccc(-c2cc(-c3ccccc3)cc(N(c3ccc(-c4ccc5oc6ccccc6c5c4)cc3)c3c(-c4ccccc4)ccc4c3oc3ccccc34)c2)cc1]

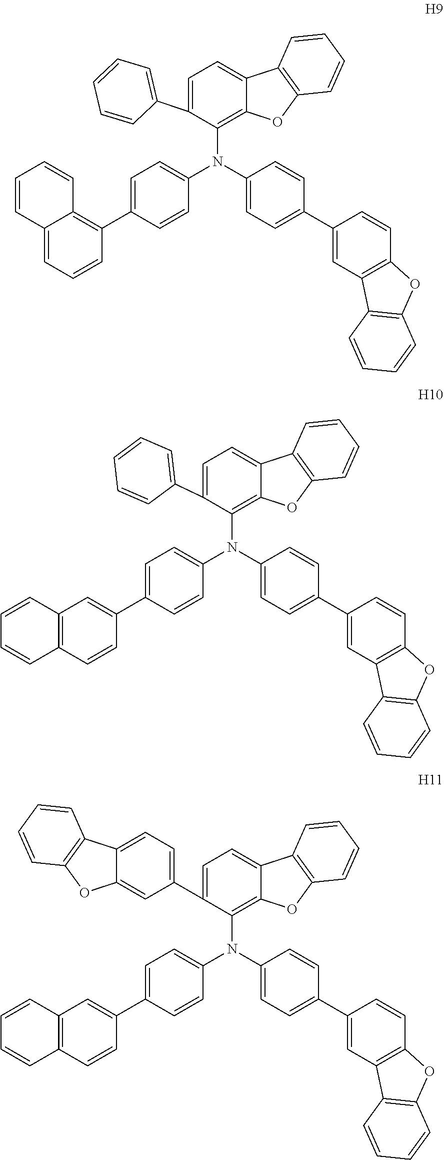 [SMILES: c1ccc(-c2ccc3c(oc4ccccc43)c2N(c2ccc(-c3ccc4oc5ccccc5c4c3)cc2)c2ccc(-c3cccc4ccccc34)cc2)cc1 ; c1ccc(-c2ccc3c(oc4ccccc43)c2N(c2ccc(-c3ccc4ccccc4c3)cc2)c2ccc(-c3ccc4oc5ccccc5c4c3)cc2)cc1 ; c1ccc2cc(-c3ccc(N(c4ccc(-c5ccc6oc7ccccc7c6c5)cc4)c4c(-c5ccc6c(c5)oc5ccccc56)ccc5c4oc4ccccc45)cc3)ccc2c1]

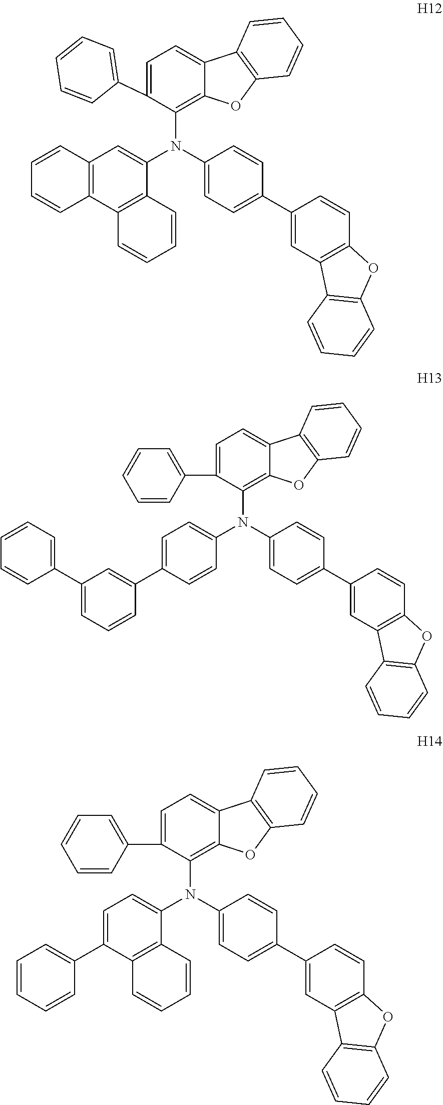 [SMILES: c1ccc(-c2ccc3c(oc4ccccc43)c2N(c2ccc(-c3ccc4oc5ccccc5c4c3)cc2)c2cc3ccccc3c3ccccc23)cc1 ; c1ccc(-c2cccc(-c3ccc(N(c4ccc(-c5ccc6oc7ccccc7c6c5)cc4)c4c(-c5ccccc5)ccc5c4oc4ccccc45)cc3)c2)cc1 ; c1ccc(-c2ccc3c(oc4ccccc43)c2N(c2ccc(-c3ccc4oc5ccccc5c4c3)cc2)c2ccc(-c3ccccc3)c3ccccc23)cc1]

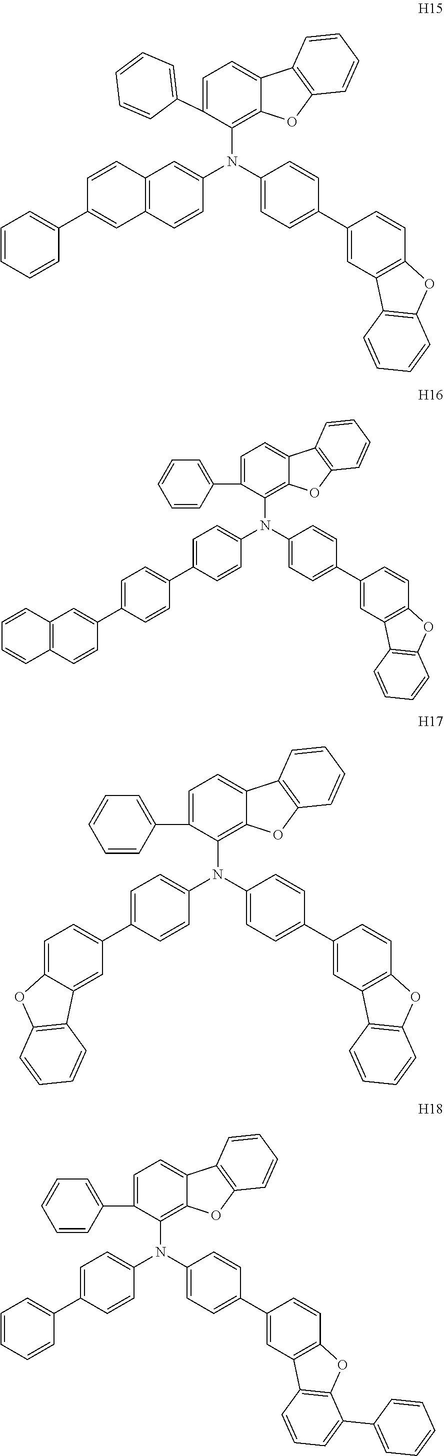 [SMILES: c1ccc(-c2ccc3cc(N(c4ccc(-c5ccc6oc7ccccc7c6c5)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3ccc4oc5ccccc5c4c3)cc2)cc1 ; c1ccc(-c2ccc3c(oc4ccccc43)c2N(c2ccc(-c3ccc4oc5ccccc5c4c3)cc2)c2ccc(-c3ccc4oc5ccccc5c4c3)cc2)cc1 ; c1ccc(-c2ccc(N(c3ccc(-c4ccc5oc6c(-c7ccccc7)cccc6c5c4)cc3)c3c(-c4ccccc4)ccc4c3oc3ccccc34)cc2)cc1]

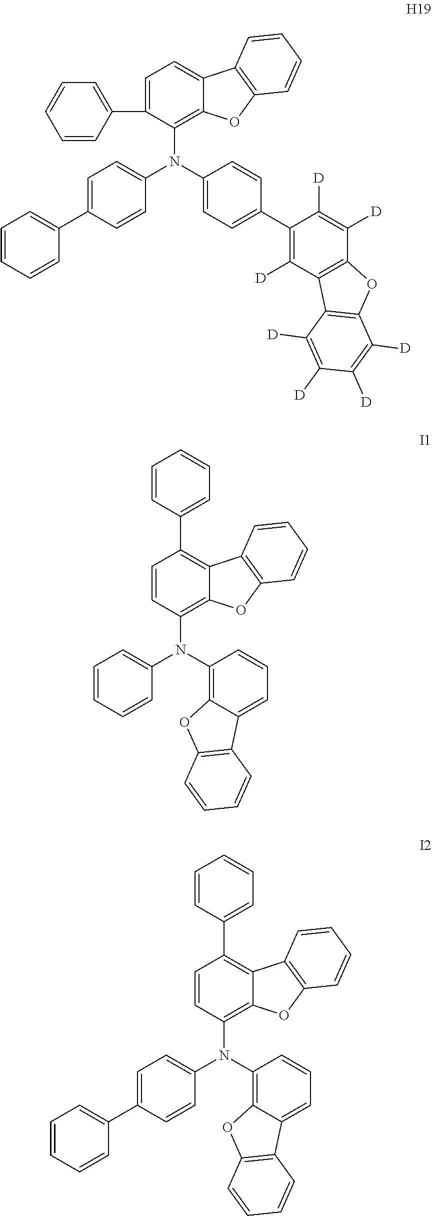 [SMILES: [2H]c1c([2H])c([2H])c2c(oc3c([2H])c([2H])c(-c4ccc(N(c5ccc(-c6ccccc6)cc5)c5c(-c6ccccc6)ccc6c5oc5ccccc56)cc4)c([2H])c32)c1[2H] ; c1ccc(-c2ccc(N(c3ccccc3)c3cccc4c3oc3ccccc34)c3oc4ccccc4c23)cc1 ; c1ccc(-c2ccc(N(c3cccc4c3oc3ccccc34)c3ccc(-c4ccccc4)c4c3oc3ccccc34)cc2)cc1]

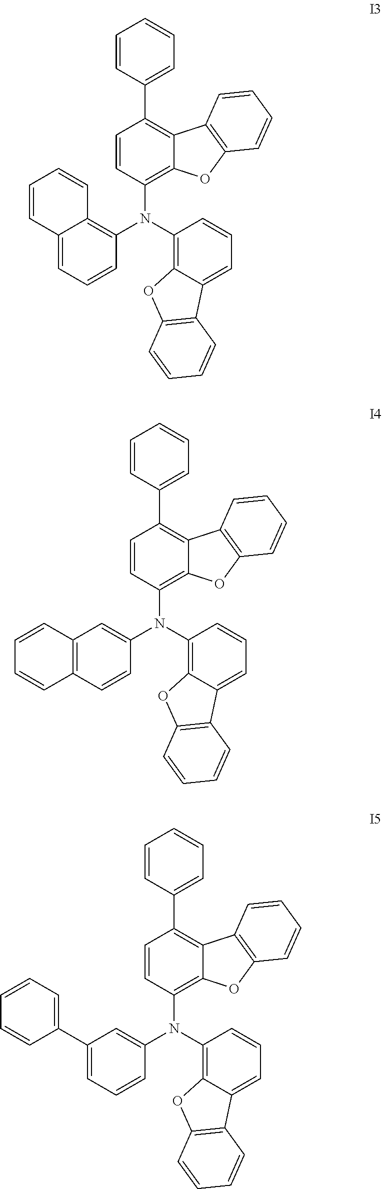

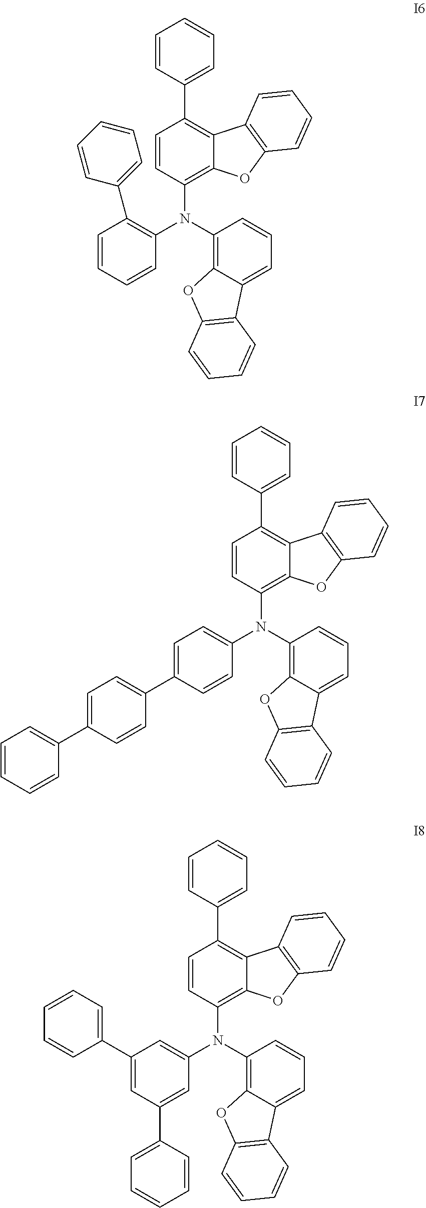 [SMILES: c1ccc(-c2ccccc2N(c2cccc3c2oc2ccccc23)c2ccc(-c3ccccc3)c3c2oc2ccccc23)cc1 ; c1ccc(-c2ccc(-c3ccc(N(c4cccc5c4oc4ccccc45)c4ccc(-c5ccccc5)c5c4oc4ccccc45)cc3)cc2)cc1 ; c1ccc(-c2cc(-c3ccccc3)cc(N(c3cccc4c3oc3ccccc34)c3ccc(-c4ccccc4)c4c3oc3ccccc34)c2)cc1]

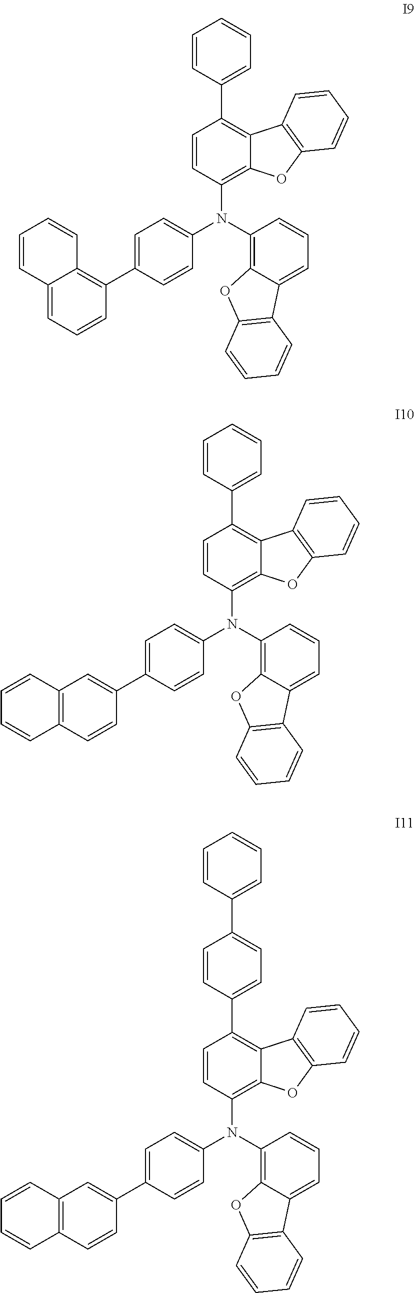 [SMILES: c1ccc(-c2ccc(N(c3ccc(-c4cccc5ccccc45)cc3)c3cccc4c3oc3ccccc34)c3oc4ccccc4c23)cc1 ; c1ccc(-c2ccc(N(c3ccc(-c4ccc5ccccc5c4)cc3)c3cccc4c3oc3ccccc34)c3oc4ccccc4c23)cc1 ; c1ccc(-c2ccc(-c3ccc(N(c4ccc(-c5ccc6ccccc6c5)cc4)c4cccc5c4oc4ccccc45)c4oc5ccccc5c34)cc2)cc1]

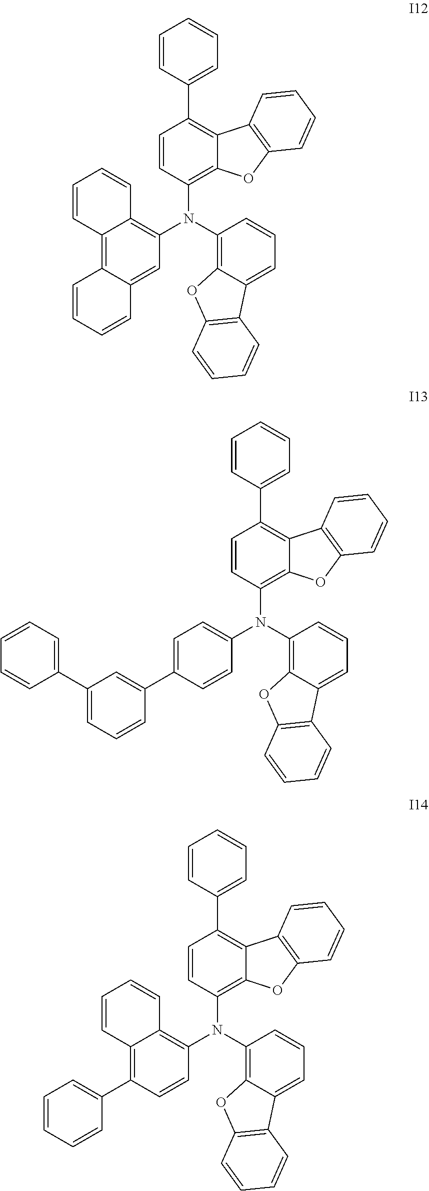 [SMILES: c1ccc(-c2ccc(N(c3cc4ccccc4c4ccccc34)c3cccc4c3oc3ccccc34)c3oc4ccccc4c23)cc1 ; c1ccc(-c2cccc(-c3ccc(N(c4cccc5c4oc4ccccc45)c4ccc(-c5ccccc5)c5c4oc4ccccc45)cc3)c2)cc1 ; c1ccc(-c2ccc(N(c3cccc4c3oc3ccccc34)c3ccc(-c4ccccc4)c4c3oc3ccccc34)c3ccccc23)cc1]

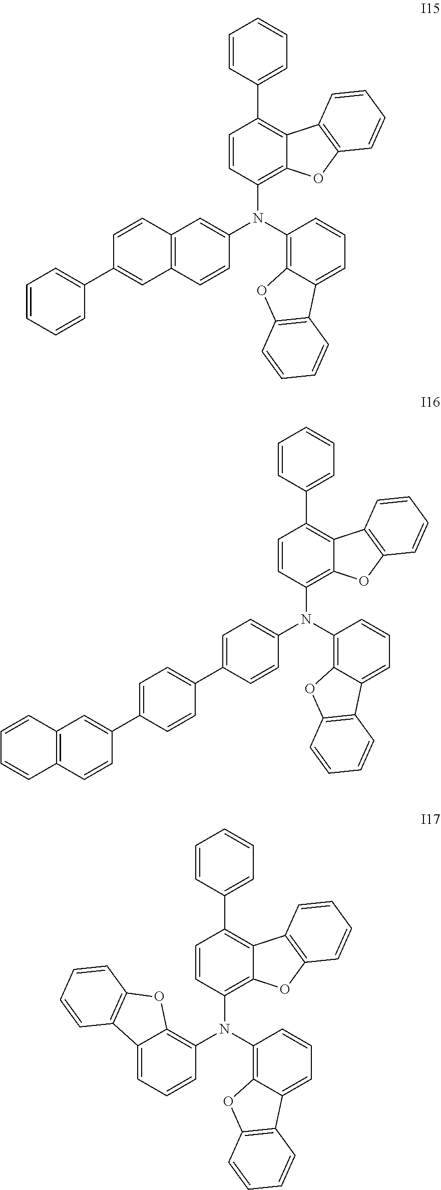 [SMILES: c1ccc(-c2ccc3cc(N(c4cccc5c4oc4ccccc45)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3cccc4c3oc3ccccc34)c3oc4ccccc4c23)cc1 ; c1ccc(-c2ccc(N(c3cccc4c3oc3ccccc34)c3cccc4c3oc3ccccc34)c3oc4ccccc4c23)cc1]

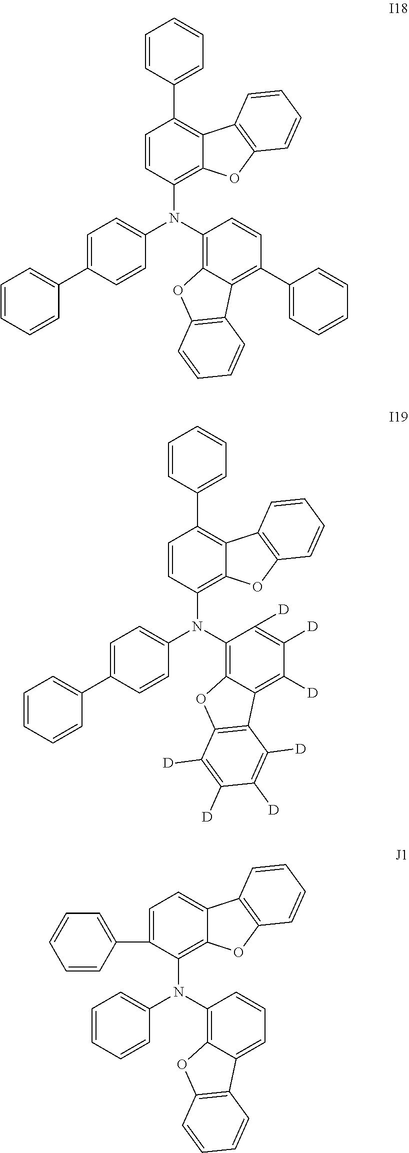 [SMILES: c1ccc(-c2ccc(N(c3ccc(-c4ccccc4)c4c3oc3ccccc34)c3ccc(-c4ccccc4)c4c3oc3ccccc34)cc2)cc1 ; [2H]c1c([2H])c([2H])c2c(oc3c(N(c4ccc(-c5ccccc5)cc4)c4ccc(-c5ccccc5)c5c4oc4ccccc45)c([2H])c([2H])c([2H])c32)c1[2H] ; c1ccc(-c2ccc3c(oc4ccccc43)c2N(c2ccccc2)c2cccc3c2oc2ccccc23)cc1]

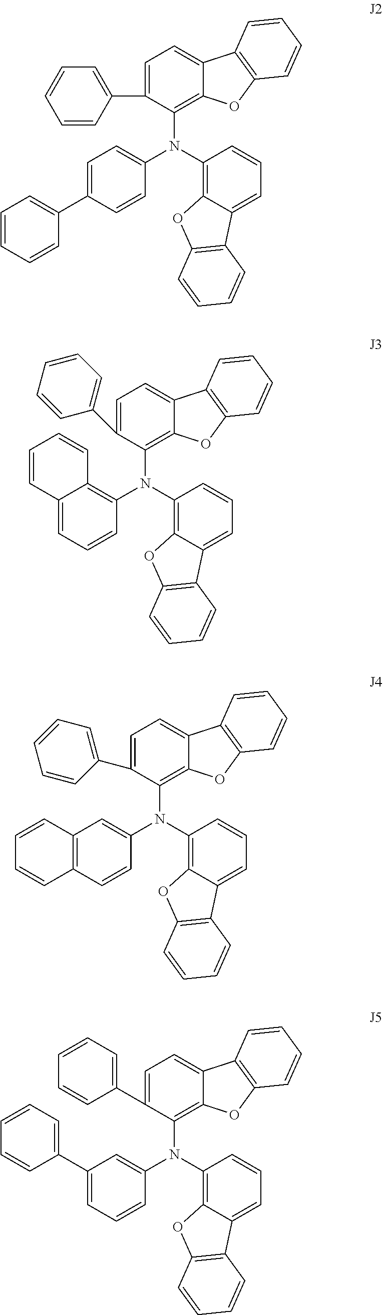 [SMILES: c1ccc(-c2ccc(N(c3cccc4c3oc3ccccc34)c3c(-c4ccccc4)ccc4c3oc3ccccc34)cc2)cc1 ; c1ccc(-c2ccc3c(oc4ccccc43)c2N(c2cccc3ccccc23)c2cccc3c2oc2ccccc23)cc1 ; c1ccc(-c2ccc3c(oc4ccccc43)c2N(c2ccc3ccccc3c2)c2cccc3c2oc2ccccc23)cc1 ; c1ccc(-c2cccc(N(c3cccc4c3oc3ccccc34)c3c(-c4ccccc4)ccc4c3oc3ccccc34)c2)cc1]

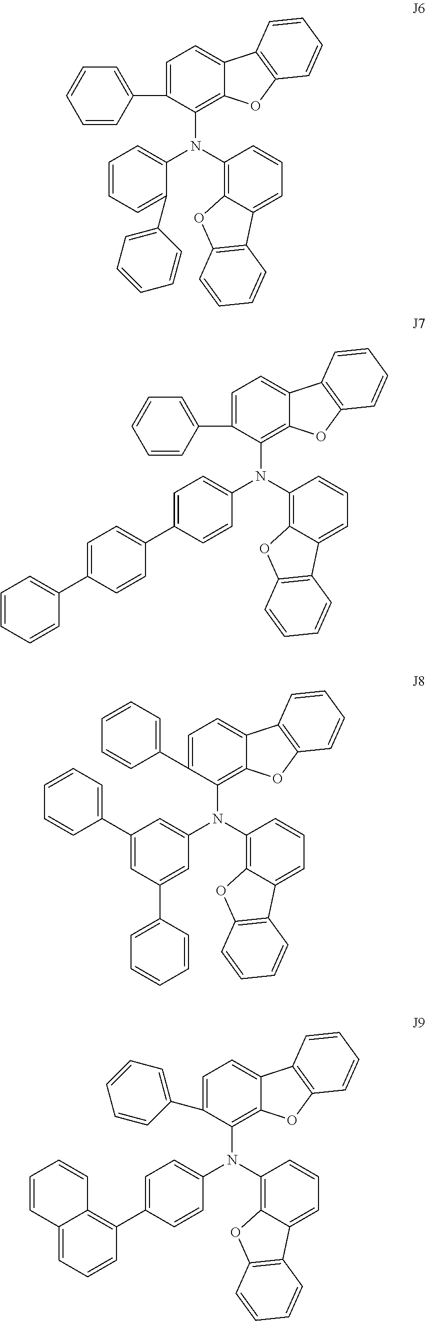 [SMILES: c1ccc(-c2ccccc2N(c2cccc3c2oc2ccccc23)c2c(-c3ccccc3)ccc3c2oc2ccccc23)cc1 ; c1ccc(-c2ccc(-c3ccc(N(c4cccc5c4oc4ccccc45)c4c(-c5ccccc5)ccc5c4oc4ccccc45)cc3)cc2)cc1 ; c1ccc(-c2cc(-c3ccccc3)cc(N(c3cccc4c3oc3ccccc34)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2cccc3c2oc2ccccc23)cc1]

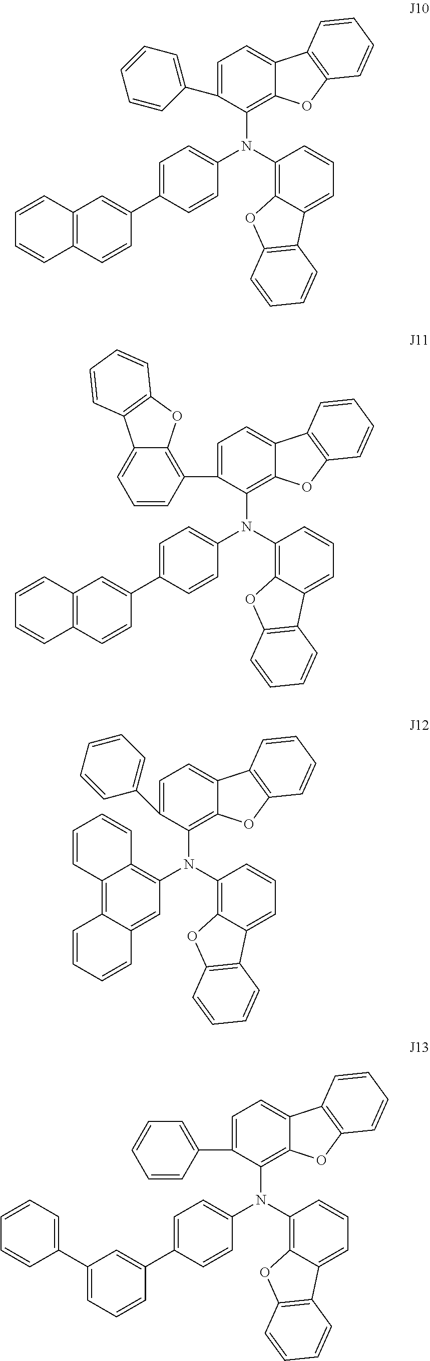 [SMILES: c1ccc(-c2ccc3c(oc4ccccc43)c2N(c2ccc(-c3ccc4ccccc4c3)cc2)c2cccc3c2oc2ccccc23)cc1 ; c1ccc2cc(-c3ccc(N(c4cccc5c4oc4ccccc45)c4c(-c5cccc6c5oc5ccccc56)ccc5c4oc4ccccc45)cc3)ccc2c1 ; c1ccc(-c2ccc3c(oc4ccccc43)c2N(c2cc3ccccc3c3ccccc23)c2cccc3c2oc2ccccc23)cc1 ; c1ccc(-c2cccc(-c3ccc(N(c4cccc5c4oc4ccccc45)c4c(-c5ccccc5)ccc5c4oc4ccccc45)cc3)c2)cc1]

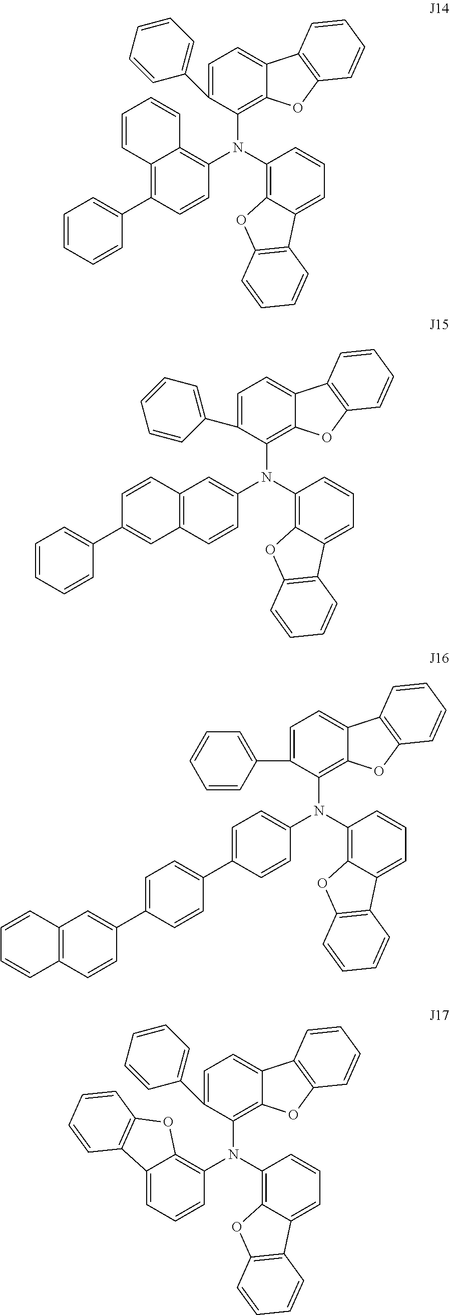 [SMILES: c1ccc(-c2ccc3c(oc4ccccc43)c2N(c2ccc(-c3ccccc3)c3ccccc23)c2cccc3c2oc2ccccc23)cc1 ; c1ccc(-c2ccc3cc(N(c4cccc5c4oc4ccccc45)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2cccc3c2oc2ccccc23)cc1 ; c1ccc(-c2ccc3c(oc4ccccc43)c2N(c2cccc3c2oc2ccccc23)c2cccc3c2oc2ccccc23)cc1]

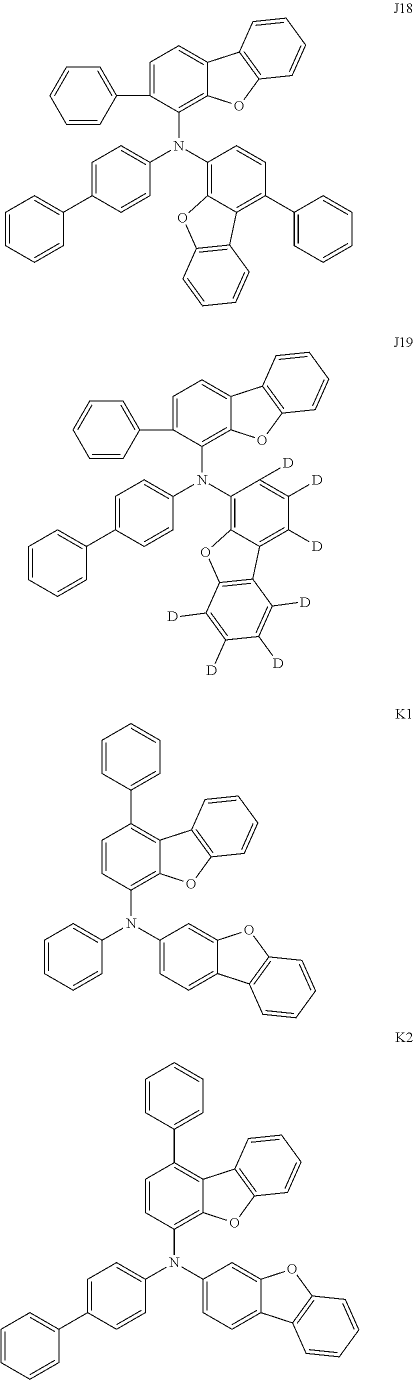 [SMILES: c1ccc(-c2ccc(N(c3c(-c4ccccc4)ccc4c3oc3ccccc34)c3ccc(-c4ccccc4)c4c3oc3ccccc34)cc2)cc1 ; [2H]c1c([2H])c([2H])c2c(oc3c(N(c4ccc(-c5ccccc5)cc4)c4c(-c5ccccc5)ccc5c4oc4ccccc45)c([2H])c([2H])c([2H])c32)c1[2H] ; c1ccc(-c2ccc(N(c3ccccc3)c3ccc4c(c3)oc3ccccc34)c3oc4ccccc4c23)cc1 ; c1ccc(-c2ccc(N(c3ccc4c(c3)oc3ccccc34)c3ccc(-c4ccccc4)c4c3oc3ccccc34)cc2)cc1]

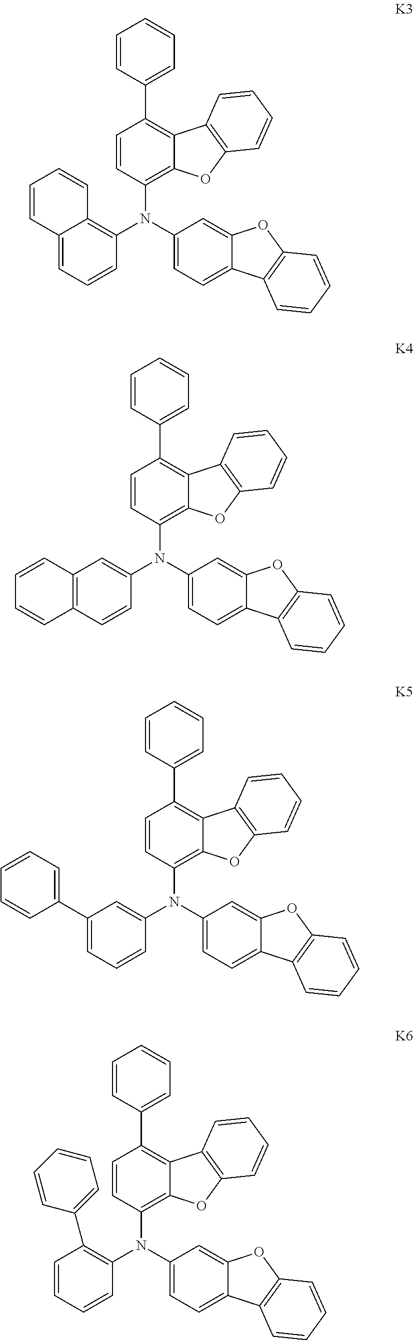 [SMILES: c1ccc(-c2ccc(N(c3ccc4c(c3)oc3ccccc34)c3cccc4ccccc34)c3oc4ccccc4c23)cc1 ; c1ccc(-c2ccc(N(c3ccc4ccccc4c3)c3ccc4c(c3)oc3ccccc34)c3oc4ccccc4c23)cc1 ; c1ccc(-c2cccc(N(c3ccc4c(c3)oc3ccccc34)c3ccc(-c4ccccc4)c4c3oc3ccccc34)c2)cc1 ; c1ccc(-c2ccccc2N(c2ccc3c(c2)oc2ccccc23)c2ccc(-c3ccccc3)c3c2oc2ccccc23)cc1]

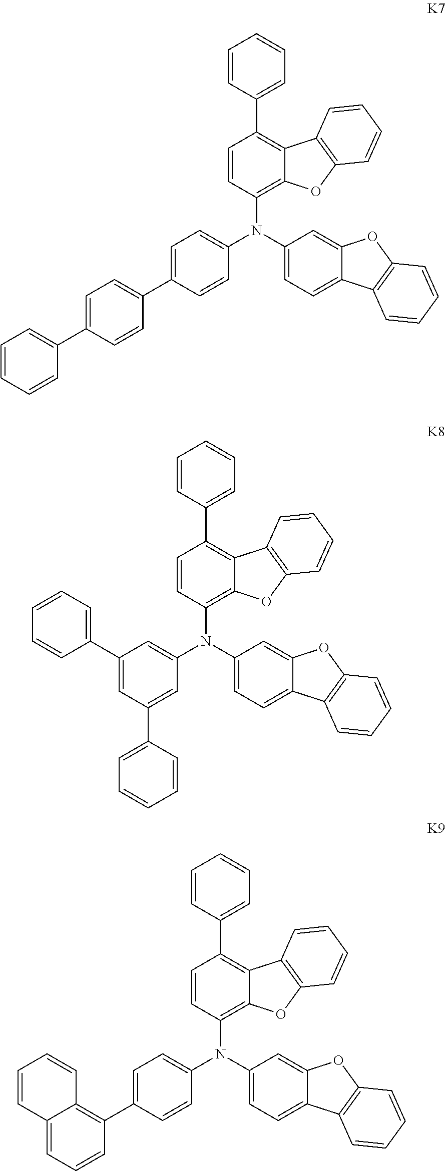 [SMILES: c1ccc(-c2ccc(-c3ccc(N(c4ccc5c(c4)oc4ccccc45)c4ccc(-c5ccccc5)c5c4oc4ccccc45)cc3)cc2)cc1 ; c1ccc(-c2cc(-c3ccccc3)cc(N(c3ccc4c(c3)oc3ccccc34)c3ccc(-c4ccccc4)c4c3oc3ccccc34)c2)cc1 ; c1ccc(-c2ccc(N(c3ccc(-c4cccc5ccccc45)cc3)c3ccc4c(c3)oc3ccccc34)c3oc4ccccc4c23)cc1]

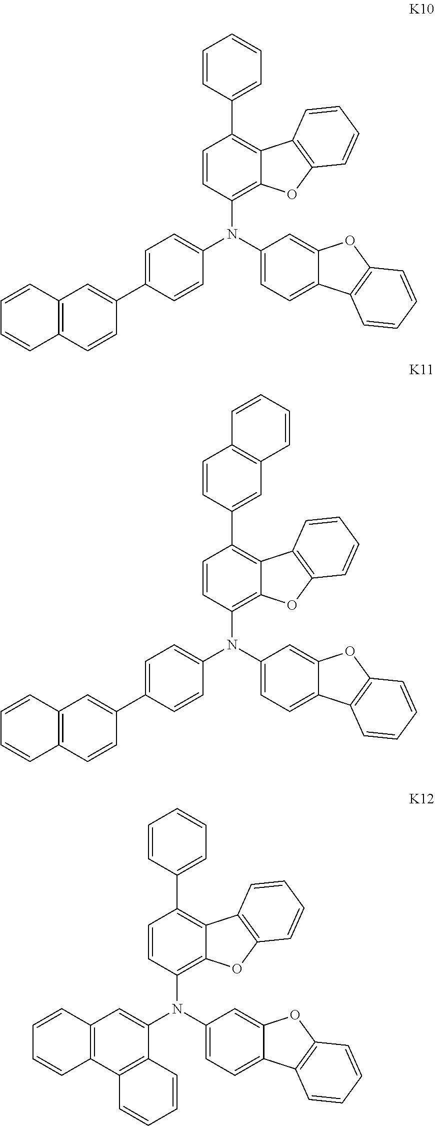 [SMILES: c1ccc(-c2ccc(N(c3ccc(-c4ccc5ccccc5c4)cc3)c3ccc4c(c3)oc3ccccc34)c3oc4ccccc4c23)cc1 ; c1ccc2cc(-c3ccc(N(c4ccc5c(c4)oc4ccccc45)c4ccc(-c5ccc6ccccc6c5)c5c4oc4ccccc45)cc3)ccc2c1 ; c1ccc(-c2ccc(N(c3ccc4c(c3)oc3ccccc34)c3cc4ccccc4c4ccccc34)c3oc4ccccc4c23)cc1]

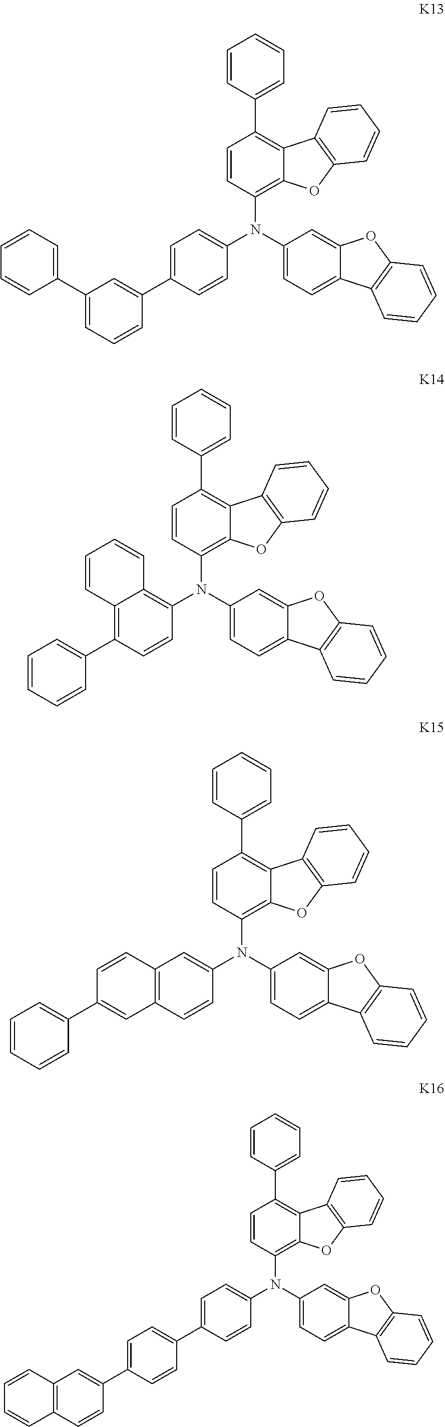 [SMILES: c1ccc(-c2cccc(-c3ccc(N(c4ccc5c(c4)oc4ccccc45)c4ccc(-c5ccccc5)c5c4oc4ccccc45)cc3)c2)cc1 ; c1ccc(-c2ccc(N(c3ccc4c(c3)oc3ccccc34)c3ccc(-c4ccccc4)c4c3oc3ccccc34)c3ccccc23)cc1 ; c1ccc(-c2ccc3cc(N(c4ccc5c(c4)oc4ccccc45)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc4c(c3)oc3ccccc34)c3oc4ccccc4c23)cc1]

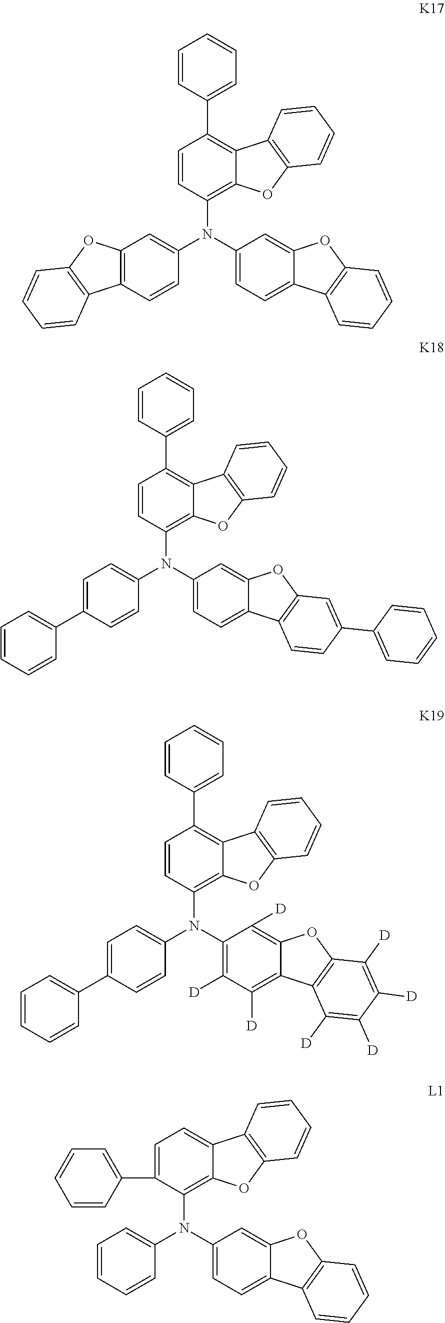 [SMILES: c1ccc(-c2ccc(N(c3ccc4c(c3)oc3ccccc34)c3ccc4c(c3)oc3ccccc34)c3oc4ccccc4c23)cc1 ; c1ccc(-c2ccc(N(c3ccc4c(c3)oc3cc(-c5ccccc5)ccc34)c3ccc(-c4ccccc4)c4c3oc3ccccc34)cc2)cc1 ; [2H]c1c([2H])c([2H])c2c(oc3c([2H])c(N(c4ccc(-c5ccccc5)cc4)c4ccc(-c5ccccc5)c5c4oc4ccccc45)c([2H])c([2H])c32)c1[2H] ; c1ccc(-c2ccc3c(oc4ccccc43)c2N(c2ccccc2)c2ccc3c(c2)oc2ccccc23)cc1]

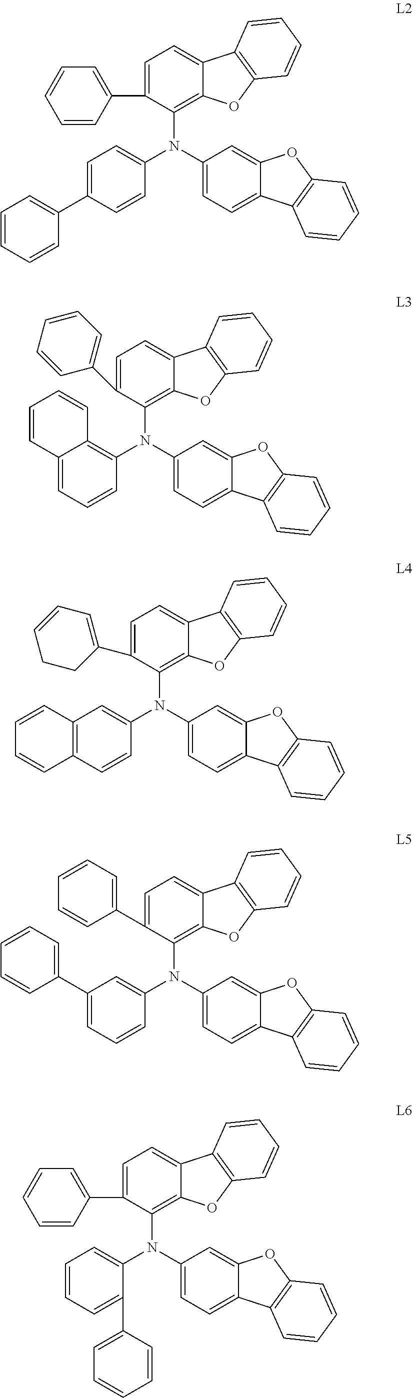 [SMILES: c1ccc(-c2ccc(N(c3ccc4c(c3)oc3ccccc34)c3c(-c4ccccc4)ccc4c3oc3ccccc34)cc2)cc1 ; c1ccc(-c2ccc3c(oc4ccccc43)c2N(c2ccc3c(c2)oc2ccccc23)c2cccc3ccccc23)cc1 ; C1=CCCC(c2ccc3c(oc4ccccc43)c2N(c2ccc3ccccc3c2)c2ccc3c(c2)oc2ccccc23)=C1 ; c1ccc(-c2cccc(N(c3ccc4c(c3)oc3ccccc34)c3c(-c4ccccc4)ccc4c3oc3ccccc34)c2)cc1 ; c1ccc(-c2ccccc2N(c2ccc3c(c2)oc2ccccc23)c2c(-c3ccccc3)ccc3c2oc2ccccc23)cc1]

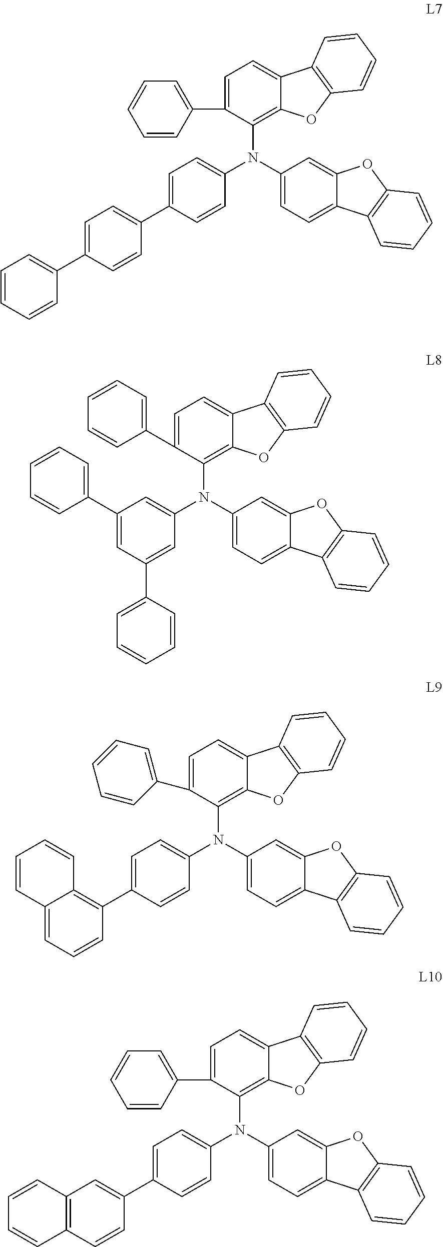 [SMILES: c1ccc(-c2ccc(-c3ccc(N(c4ccc5c(c4)oc4ccccc45)c4c(-c5ccccc5)ccc5c4oc4ccccc45)cc3)cc2)cc1 ; c1ccc(-c2cc(-c3ccccc3)cc(N(c3ccc4c(c3)oc3ccccc34)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc3c(c2)oc2ccccc23)cc1 ; c1ccc(-c2ccc3c(oc4ccccc43)c2N(c2ccc(-c3ccc4ccccc4c3)cc2)c2ccc3c(c2)oc2ccccc23)cc1]

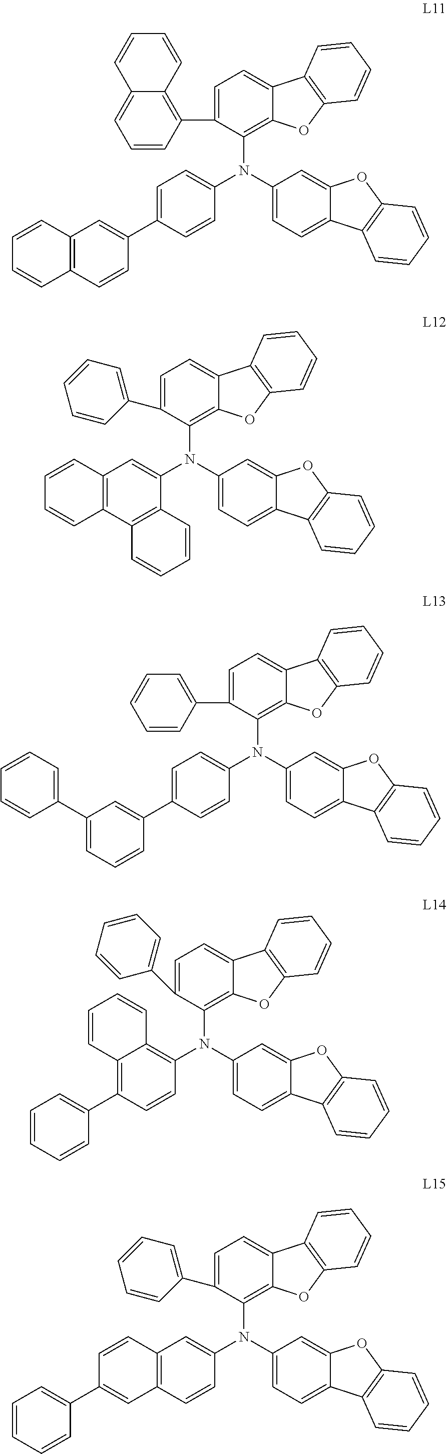 [SMILES: c1ccc2cc(-c3ccc(N(c4ccc5c(c4)oc4ccccc45)c4c(-c5cccc6ccccc56)ccc5c4oc4ccccc45)cc3)ccc2c1 ; c1ccc(-c2ccc3c(oc4ccccc43)c2N(c2ccc3c(c2)oc2ccccc23)c2cc3ccccc3c3ccccc23)cc1 ; c1ccc(-c2cccc(-c3ccc(N(c4ccc5c(c4)oc4ccccc45)c4c(-c5ccccc5)ccc5c4oc4ccccc45)cc3)c2)cc1 ; c1ccc(-c2ccc3c(oc4ccccc43)c2N(c2ccc3c(c2)oc2ccccc23)c2ccc(-c3ccccc3)c3ccccc23)cc1 ; c1ccc(-c2ccc3cc(N(c4ccc5c(c4)oc4ccccc45)c4c(-c5ccccc5)ccc5c4oc4ccccc45)ccc3c2)cc1]

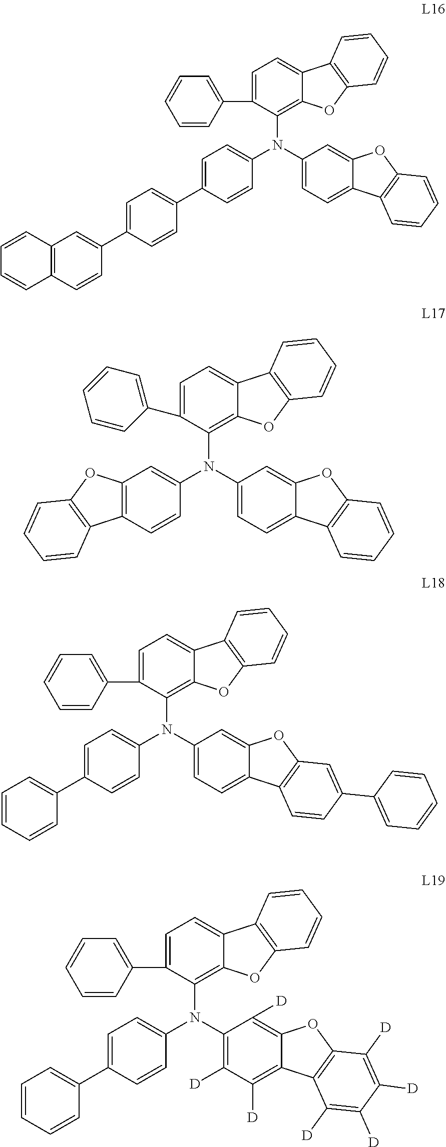 [SMILES: c1ccc(-c2ccc3c(oc4ccccc43)c2N(c2ccc(-c3ccc(-c4ccc5ccccc5c4)cc3)cc2)c2ccc3c(c2)oc2ccccc23)cc1 ; c1ccc(-c2ccc3c(oc4ccccc43)c2N(c2ccc3c(c2)oc2ccccc23)c2ccc3c(c2)oc2ccccc23)cc1 ; c1ccc(-c2ccc(N(c3ccc4c(c3)oc3cc(-c5ccccc5)ccc34)c3c(-c4ccccc4)ccc4c3oc3ccccc34)cc2)cc1 ; [2H]c1c([2H])c([2H])c2c(oc3c([2H])c(N(c4ccc(-c5ccccc5)cc4)c4c(-c5ccccc5)ccc5c4oc4ccccc45)c([2H])c([2H])c32)c1[2H]]

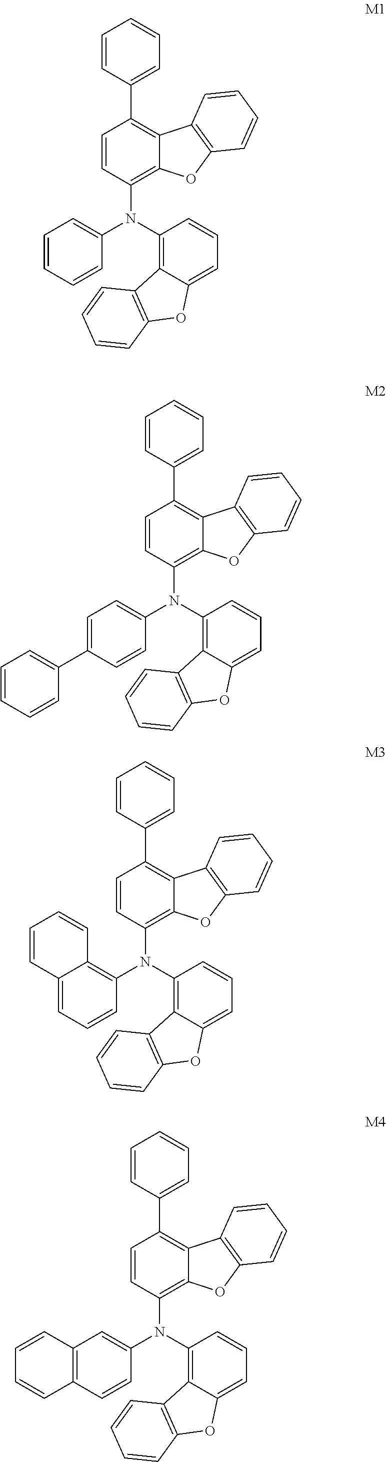

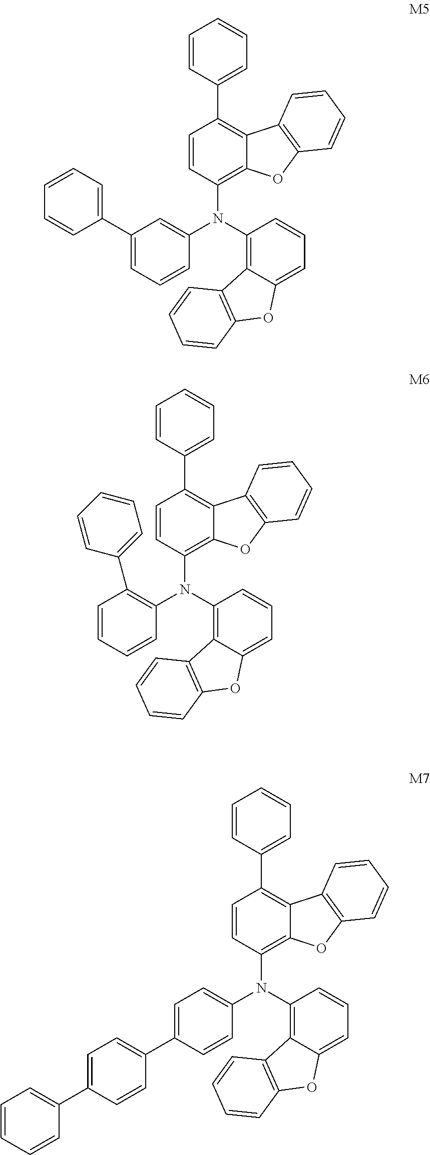

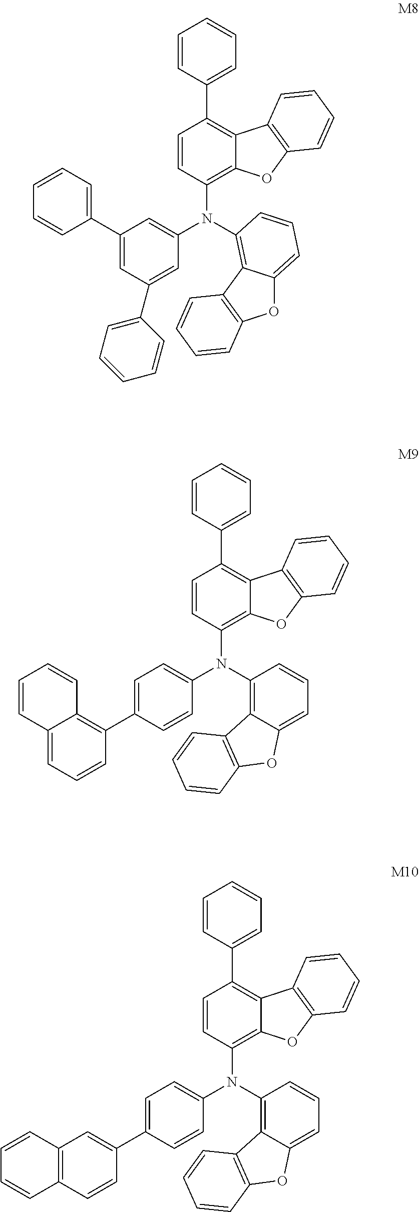 [SMILES: c1ccc(-c2cc(-c3ccccc3)cc(N(c3ccc(-c4ccccc4)c4c3oc3ccccc34)c3cccc4oc5ccccc5c34)c2)cc1 ; c1ccc(-c2ccc(N(c3ccc(-c4cccc5ccccc45)cc3)c3cccc4oc5ccccc5c34)c3oc4ccccc4c23)cc1 ; c1ccc(-c2ccc(N(c3ccc(-c4ccc5ccccc5c4)cc3)c3cccc4oc5ccccc5c34)c3oc4ccccc4c23)cc1]

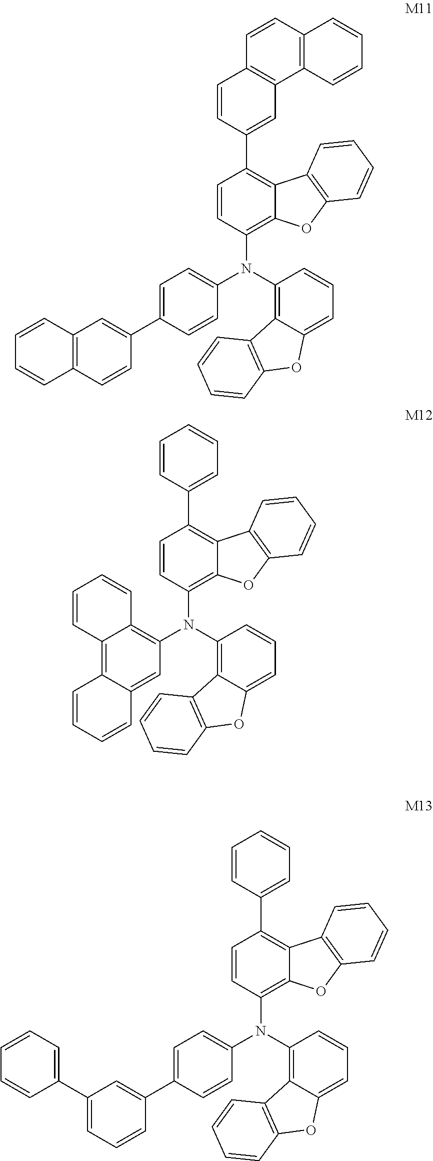

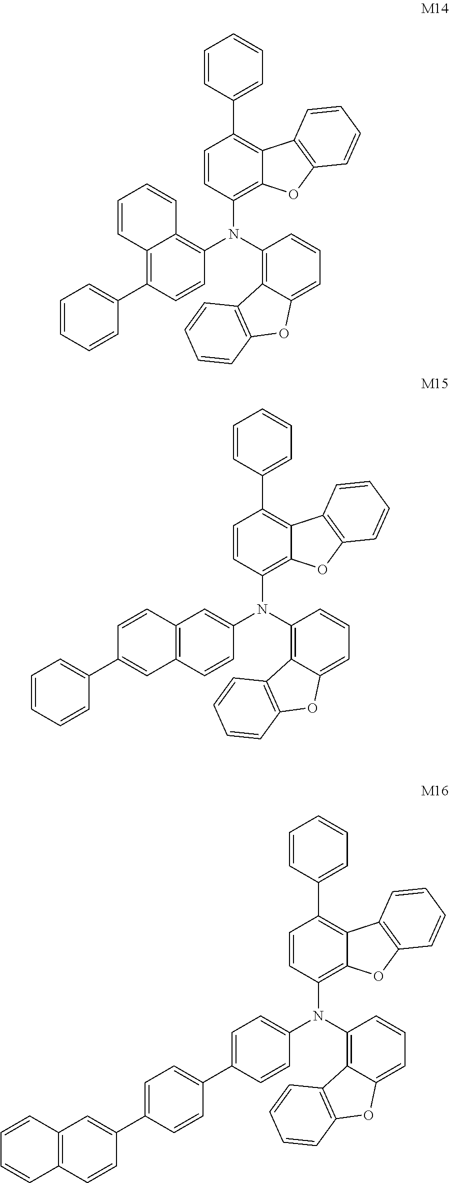 [SMILES: c1ccc(-c2ccc(N(c3ccc(-c4ccccc4)c4c3oc3ccccc34)c3cccc4oc5ccccc5c34)c3ccccc23)cc1 ; c1ccc(-c2ccc3cc(N(c4ccc(-c5ccccc5)c5c4oc4ccccc45)c4cccc5oc6ccccc6c45)ccc3c2)cc1 ; c1ccc(-c2ccc(N(c3ccc(-c4ccc(-c5ccc6ccccc6c5)cc4)cc3)c3cccc4oc5ccccc5c34)c3oc4ccccc4c23)cc1]

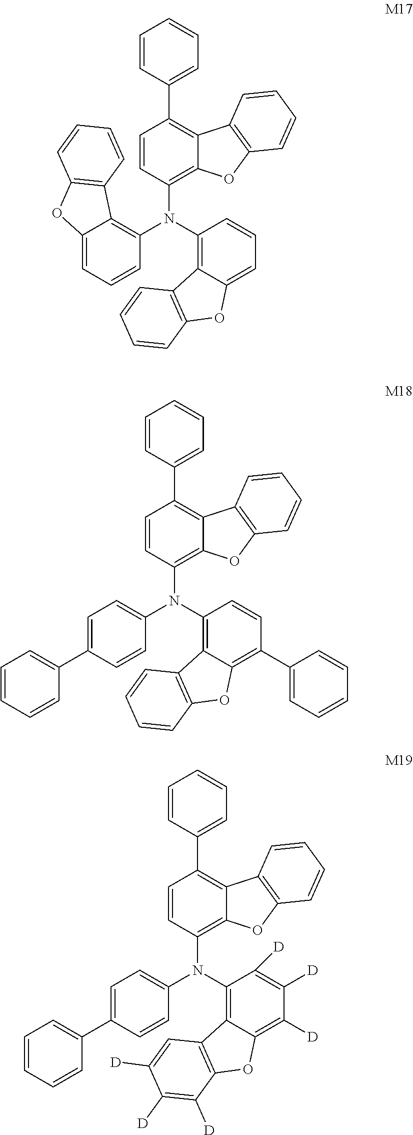 [SMILES: c1ccc(-c2ccc(N(c3cccc4oc5ccccc5c34)c3cccc4oc5ccccc5c34)c3oc4ccccc4c23)cc1 ; c1ccc(-c2ccc(N(c3ccc(-c4ccccc4)c4c3oc3ccccc34)c3ccc(-c4ccccc4)c4oc5ccccc5c34)cc2)cc1 ; [2H]c1cc2c(oc3c([2H])c([2H])c([2H])c(N(c4ccc(-c5ccccc5)cc4)c4ccc(-c5ccccc5)c5c4oc4ccccc45)c32)c([2H])c1[2H]]

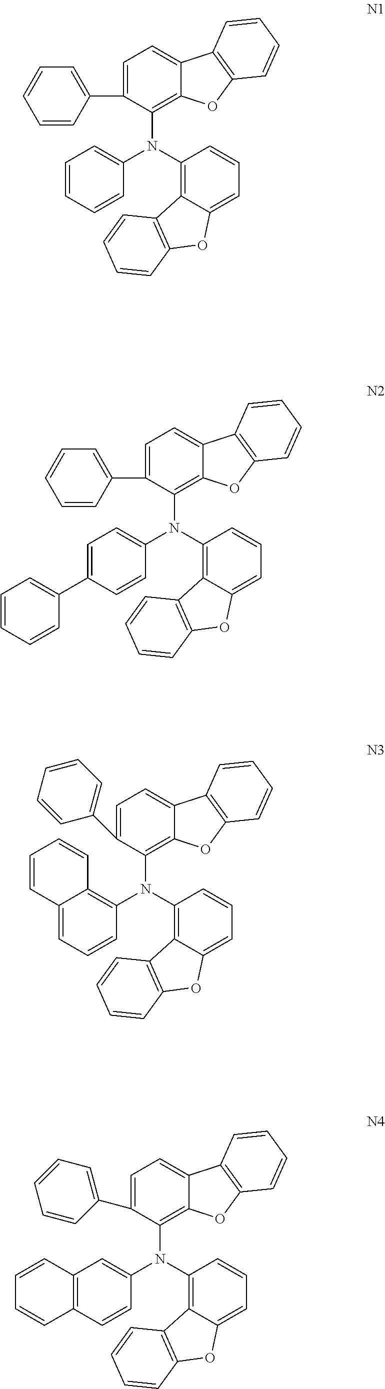 [SMILES: c1ccc(-c2ccc3c(oc4ccccc43)c2N(c2ccccc2)c2cccc3oc4ccccc4c23)cc1 ; c1ccc(-c2ccc(N(c3c(-c4ccccc4)ccc4c3oc3ccccc34)c3cccc4oc5ccccc5c34)cc2)cc1 ; c1ccc(-c2ccc3c(oc4ccccc43)c2N(c2cccc3ccccc23)c2cccc3oc4ccccc4c23)cc1 ; c1ccc(-c2ccc3c(oc4ccccc43)c2N(c2ccc3ccccc3c2)c2cccc3oc4ccccc4c23)cc1]

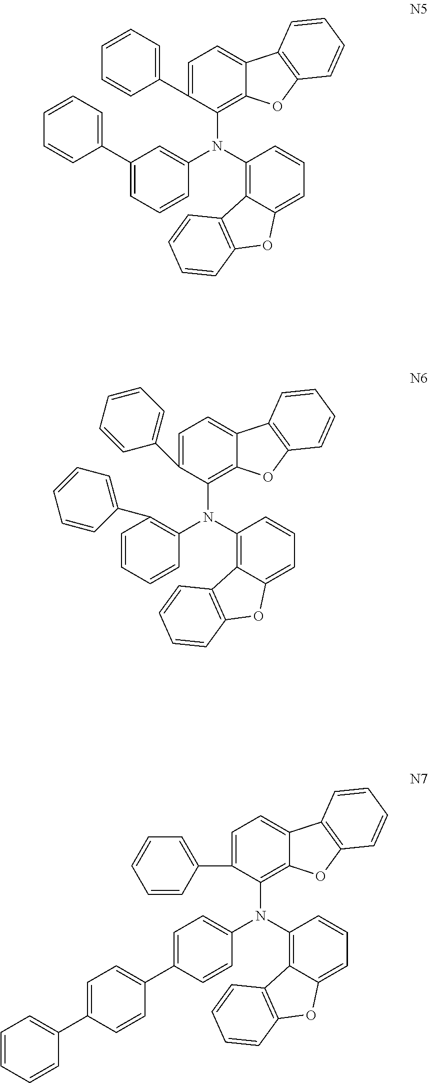 [SMILES: c1ccc(-c2cccc(N(c3c(-c4ccccc4)ccc4c3oc3ccccc34)c3cccc4oc5ccccc5c34)c2)cc1 ; c1ccc(-c2ccccc2N(c2c(-c3ccccc3)ccc3c2oc2ccccc23)c2cccc3oc4ccccc4c23)cc1 ; c1ccc(-c2ccc(-c3ccc(N(c4c(-c5ccccc5)ccc5c4oc4ccccc45)c4cccc5oc6ccccc6c45)cc3)cc2)cc1]

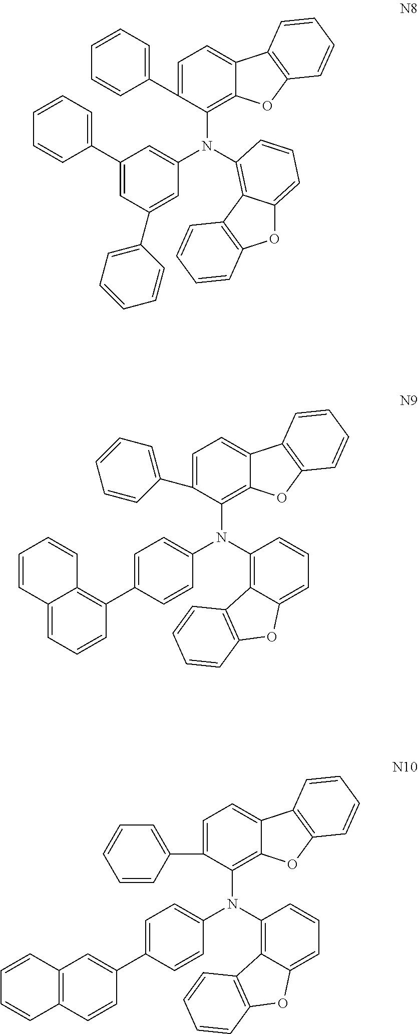 [SMILES: c1ccc(-c2cc(-c3ccccc3)cc(N(c3c(-c4ccccc4)ccc4c3oc3ccccc34)c3cccc4oc5ccccc5c34)c2)cc1 ; c1ccc(-c2ccc3c(oc4ccccc43)c2N(c2ccc(-c3cccc4ccccc34)cc2)c2cccc3oc4ccccc4c23)cc1 ; c1ccc(-c2ccc3c(oc4ccccc43)c2N(c2ccc(-c3ccc4ccccc4c3)cc2)c2cccc3oc4ccccc4c23)cc1]

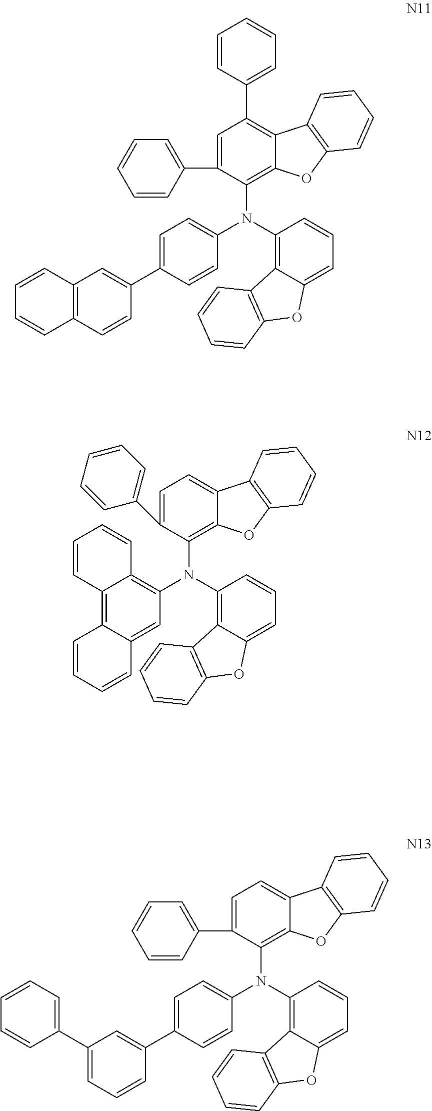 [SMILES: c1ccc(-c2cc(-c3ccccc3)c3c(oc4ccccc43)c2N(c2ccc(-c3ccc4ccccc4c3)cc2)c2cccc3oc4ccccc4c23)cc1 ; c1ccc(-c2ccc3c(oc4ccccc43)c2N(c2cc3ccccc3c3ccccc23)c2cccc3oc4ccccc4c23)cc1 ; c1ccc(-c2cccc(-c3ccc(N(c4c(-c5ccccc5)ccc5c4oc4ccccc45)c4cccc5oc6ccccc6c45)cc3)c2)cc1]

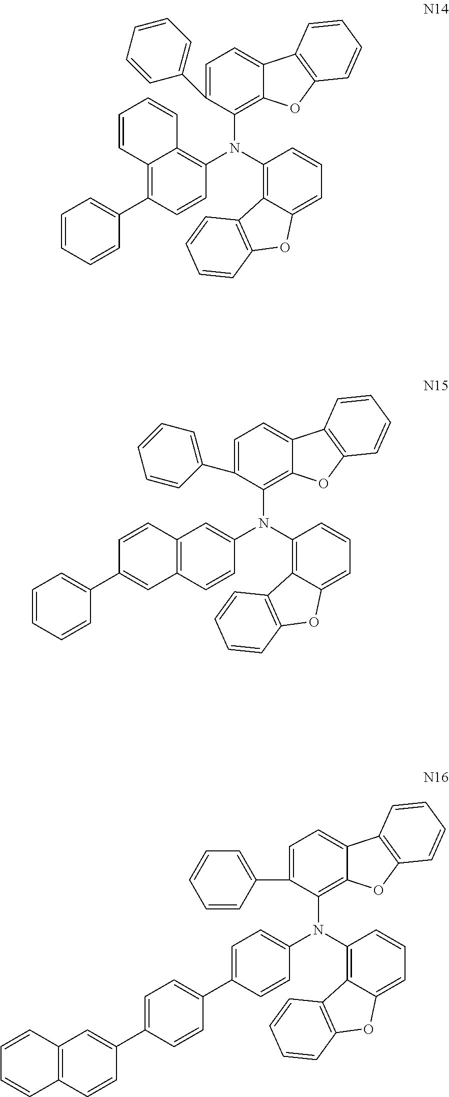 [SMILES: c1ccc(-c2ccc3c(oc4ccccc43)c2N(c2ccc(-c3ccccc3)c3ccccc23)c2cccc3oc4ccccc4c23)cc1 ; c1ccc(-c2ccc3cc(N(c4c(-c5ccccc5)ccc5c4oc4ccccc45)c4cccc5oc6ccccc6c45)ccc3c2)cc1 ; c1ccc(-c2ccc3c(oc4ccccc43)c2N(c2ccc(-c3ccc(-c4ccc5ccccc5c4)cc3)cc2)c2cccc3oc4ccccc4c23)cc1]

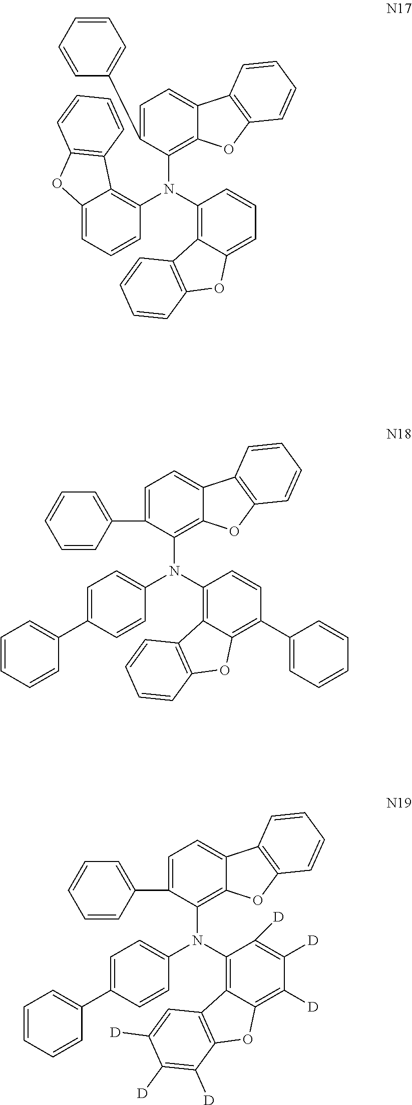 [SMILES: c1ccc(-c2ccc3c(oc4ccccc43)c2N(c2cccc3oc4ccccc4c23)c2cccc3oc4ccccc4c23)cc1 ; c1ccc(-c2ccc(N(c3c(-c4ccccc4)ccc4c3oc3ccccc34)c3ccc(-c4ccccc4)c4oc5ccccc5c34)cc2)cc1 ; [2H]c1cc2c(oc3c([2H])c([2H])c([2H])c(N(c4ccc(-c5ccccc5)cc4)c4c(-c5ccccc5)ccc5c4oc4ccccc45)c32)c([2H])c1[2H]]

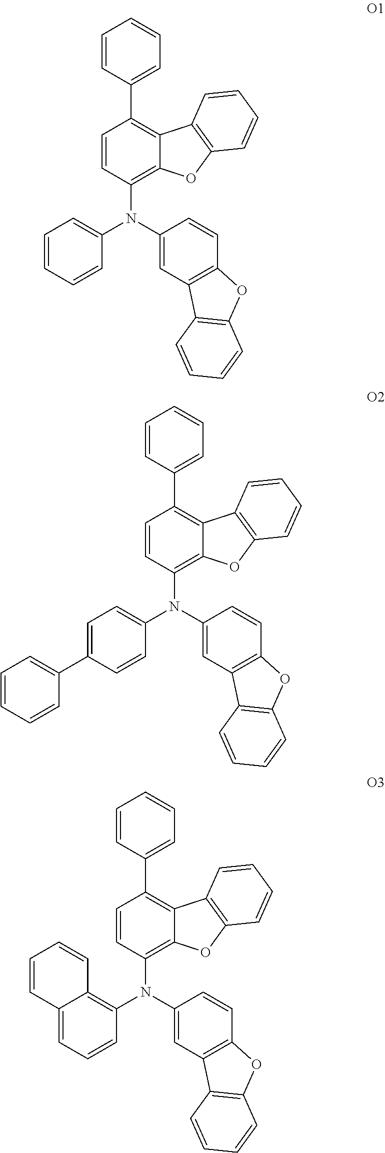

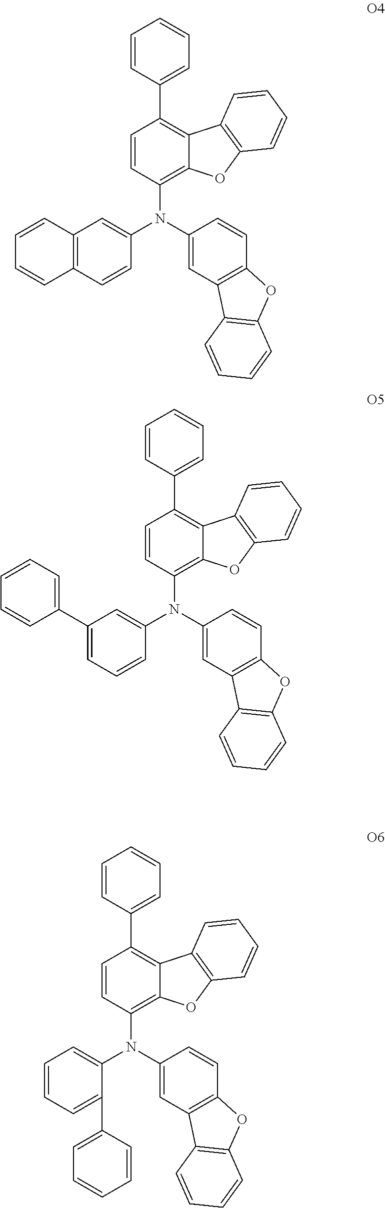 [SMILES: c1ccc(-c2ccc(N(c3ccc4ccccc4c3)c3ccc4oc5ccccc5c4c3)c3oc4ccccc4c23)cc1 ; c1ccc(-c2cccc(N(c3ccc4oc5ccccc5c4c3)c3ccc(-c4ccccc4)c4c3oc3ccccc34)c2)cc1 ; c1ccc(-c2ccccc2N(c2ccc3oc4ccccc4c3c2)c2ccc(-c3ccccc3)c3c2oc2ccccc23)cc1]

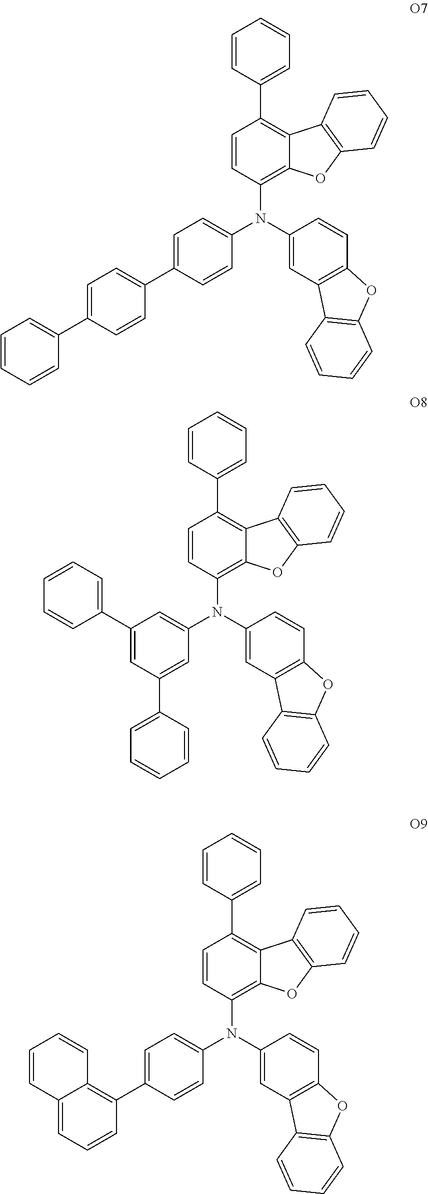 [SMILES: c1ccc(-c2ccc(-c3ccc(N(c4ccc5oc6ccccc6c5c4)c4ccc(-c5ccccc5)c5c4oc4ccccc45)cc3)cc2)cc1 ; c1ccc(-c2cc(-c3ccccc3)cc(N(c3ccc4oc5ccccc5c4c3)c3ccc(-c4ccccc4)c4c3oc3ccccc34)c2)cc1 ; c1ccc(-c2ccc(N(c3ccc(-c4cccc5ccccc45)cc3)c3ccc4oc5ccccc5c4c3)c3oc4ccccc4c23)cc1]

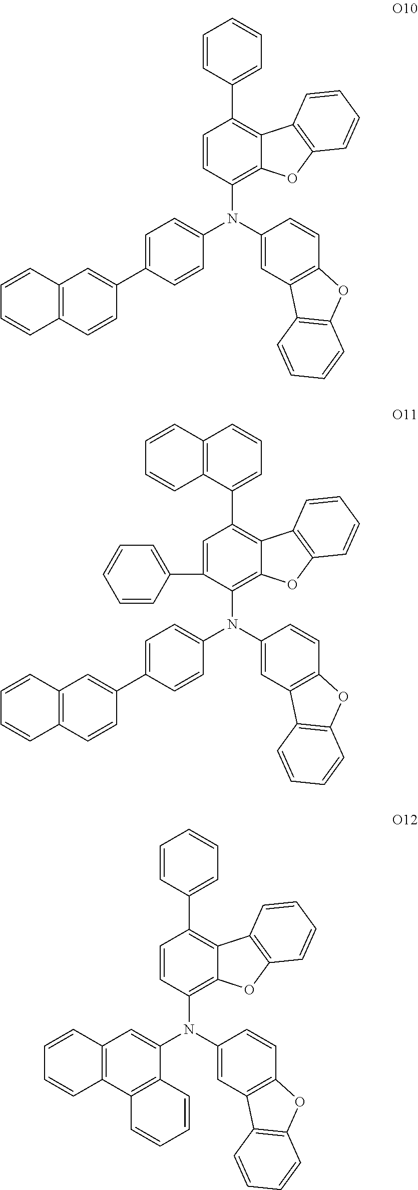 [SMILES: c1ccc(-c2ccc(N(c3ccc(-c4ccc5ccccc5c4)cc3)c3ccc4oc5ccccc5c4c3)c3oc4ccccc4c23)cc1 ; c1ccc(-c2cc(-c3cccc4ccccc34)c3c(oc4ccccc43)c2N(c2ccc(-c3ccc4ccccc4c3)cc2)c2ccc3oc4ccccc4c3c2)cc1 ; c1ccc(-c2ccc(N(c3ccc4oc5ccccc5c4c3)c3cc4ccccc4c4ccccc34)c3oc4ccccc4c23)cc1]

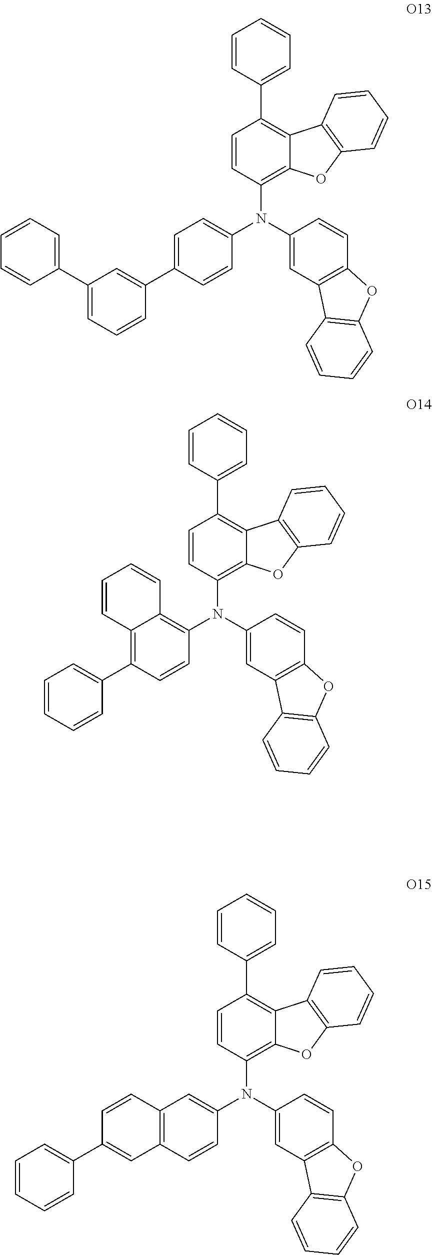 [SMILES: c1ccc(-c2cccc(-c3ccc(N(c4ccc5oc6ccccc6c5c4)c4ccc(-c5ccccc5)c5c4oc4ccccc45)cc3)c2)cc1 ; c1ccc(-c2ccc(N(c3ccc4oc5ccccc5c4c3)c3ccc(-c4ccccc4)c4c3oc3ccccc34)c3ccccc23)cc1 ; c1ccc(-c2ccc3cc(N(c4ccc5oc6ccccc6c5c4)c4ccc(-c5ccccc5)c5c4oc4ccccc45)ccc3c2)cc1]

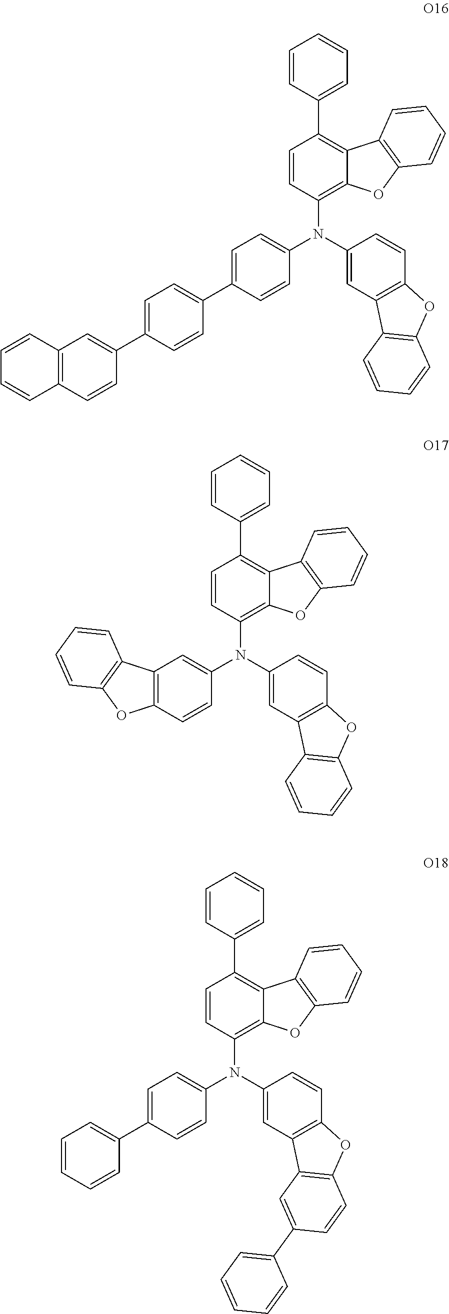 [SMILES: c1ccc(-c2ccc(N(c3ccc(-c4ccc(-c5ccc6ccccc6c5)cc4)cc3)c3ccc4oc5ccccc5c4c3)c3oc4ccccc4c23)cc1 ; c1ccc(-c2ccc(N(c3ccc4oc5ccccc5c4c3)c3ccc4oc5ccccc5c4c3)c3oc4ccccc4c23)cc1 ; c1ccc(-c2ccc(N(c3ccc4oc5ccc(-c6ccccc6)cc5c4c3)c3ccc(-c4ccccc4)c4c3oc3ccccc34)cc2)cc1]

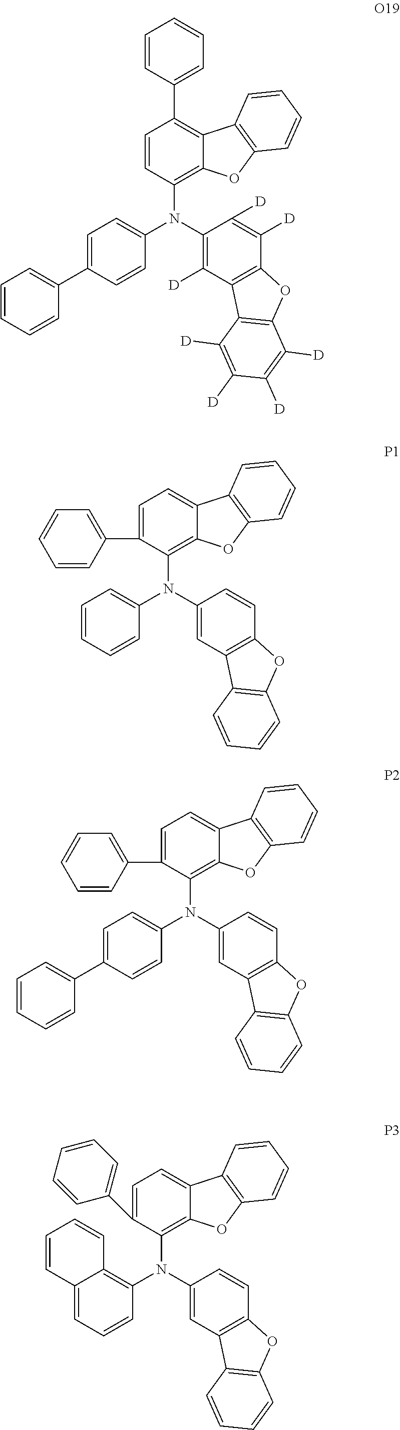

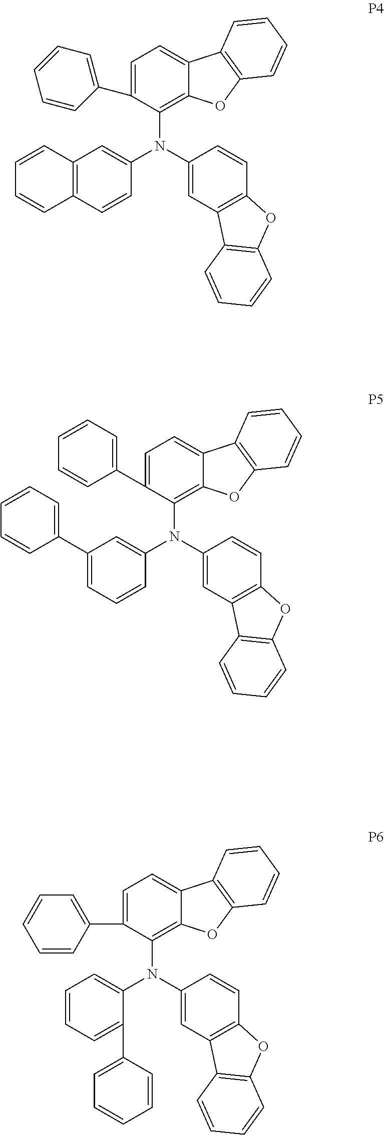 [SMILES: c1ccc(-c2ccc3c(oc4ccccc43)c2N(c2ccc3ccccc3c2)c2ccc3oc4ccccc4c3c2)cc1 ; c1ccc(-c2cccc(N(c3ccc4oc5ccccc5c4c3)c3c(-c4ccccc4)ccc4c3oc3ccccc34)c2)cc1 ; c1ccc(-c2ccccc2N(c2ccc3oc4ccccc4c3c2)c2c(-c3ccccc3)ccc3c2oc2ccccc23)cc1]

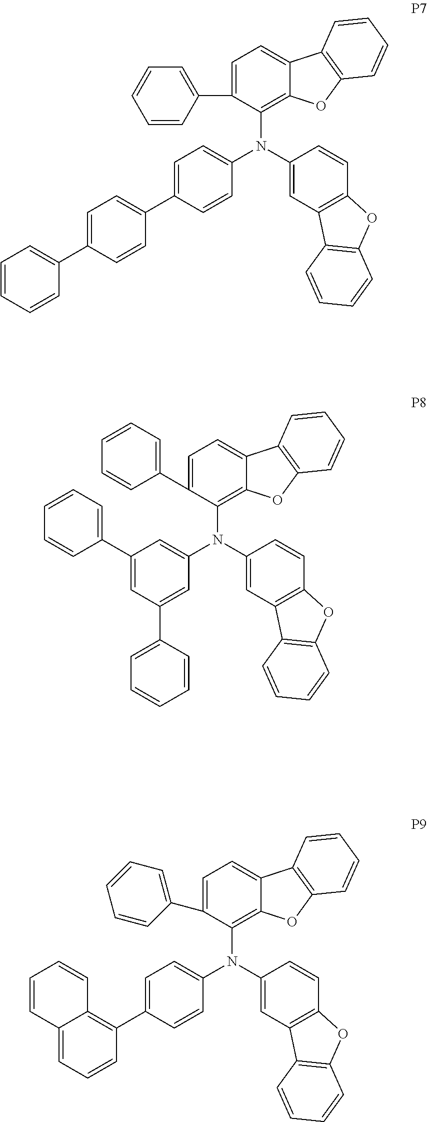 [SMILES: c1ccc(-c2ccc(-c3ccc(N(c4ccc5oc6ccccc6c5c4)c4c(-c5ccccc5)ccc5c4oc4ccccc45)cc3)cc2)cc1 ; c1ccc(-c2cc(-c3ccccc3)cc(N(c3ccc4oc5ccccc5c4c3)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc3oc4ccccc4c3c2)cc1]

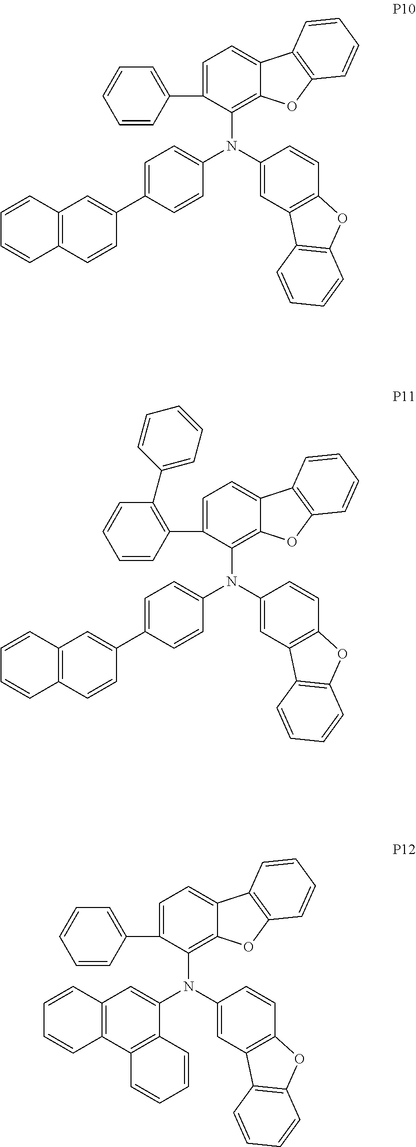 [SMILES: c1ccc(-c2ccc3c(oc4ccccc43)c2N(c2ccc(-c3ccc4ccccc4c3)cc2)c2ccc3oc4ccccc4c3c2)cc1 ; c1ccc(-c2ccccc2-c2ccc3c(oc4ccccc43)c2N(c2ccc(-c3ccc4ccccc4c3)cc2)c2ccc3oc4ccccc4c3c2)cc1 ; c1ccc(-c2ccc3c(oc4ccccc43)c2N(c2ccc3oc4ccccc4c3c2)c2cc3ccccc3c3ccccc23)cc1]

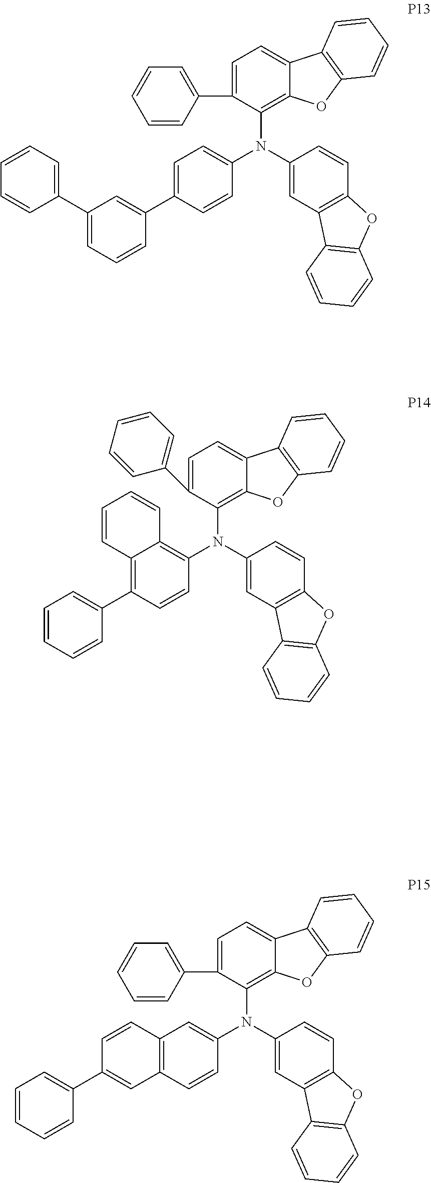 [SMILES: c1ccc(-c2cccc(-c3ccc(N(c4ccc5oc6ccccc6c5c4)c4c(-c5ccccc5)ccc5c4oc4ccccc45)cc3)c2)cc1 ; c1ccc(-c2ccc3c(oc4ccccc43)c2N(c2ccc3oc4ccccc4c3c2)c2ccc(-c3ccccc3)c3ccccc23)cc1 ; c1ccc(-c2ccc3cc(N(c4ccc5oc6ccccc6c5c4)c4c(-c5ccccc5)ccc5c4oc4ccccc45)ccc3c2)cc1]

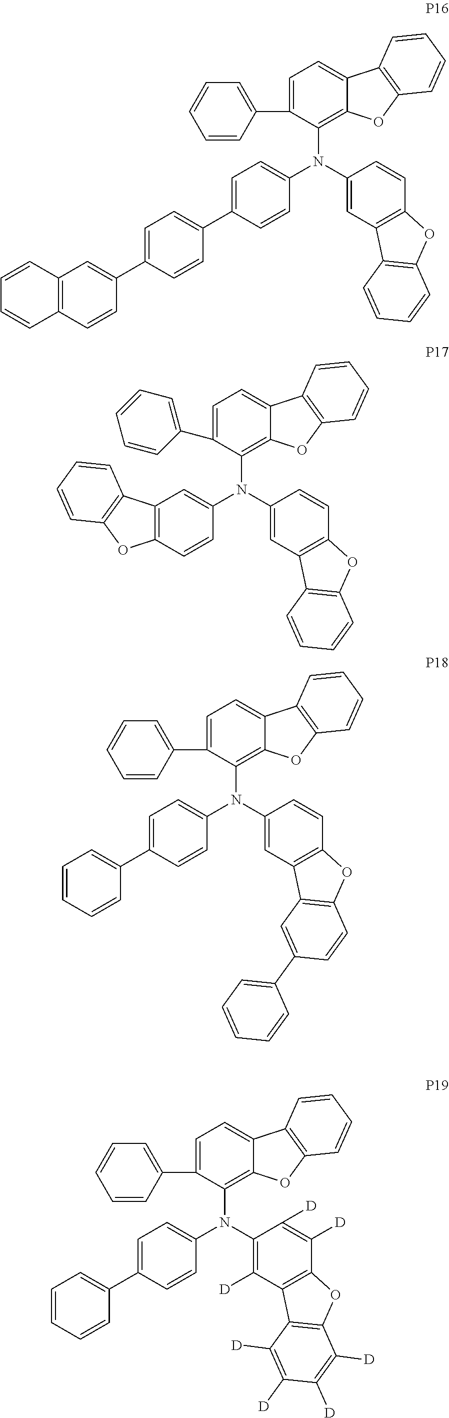

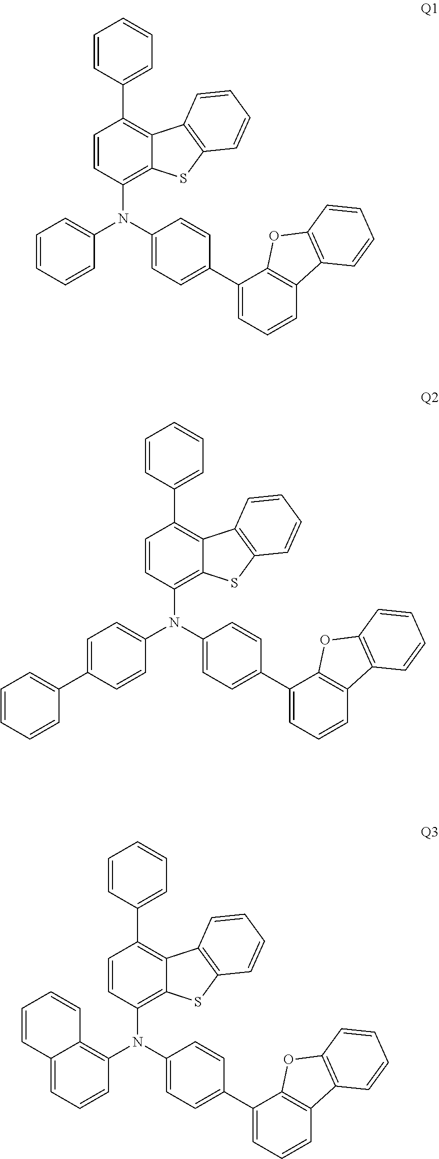 [SMILES: c1ccc(-c2ccc(N(c3ccccc3)c3ccc(-c4cccc5c4oc4ccccc45)cc3)c3sc4ccccc4c23)cc1 ; c1ccc(-c2ccc(N(c3ccc(-c4cccc5c4oc4ccccc45)cc3)c3ccc(-c4ccccc4)c4c3sc3ccccc34)cc2)cc1 ; c1ccc(-c2ccc(N(c3ccc(-c4cccc5c4oc4ccccc45)cc3)c3cccc4ccccc34)c3sc4ccccc4c23)cc1]

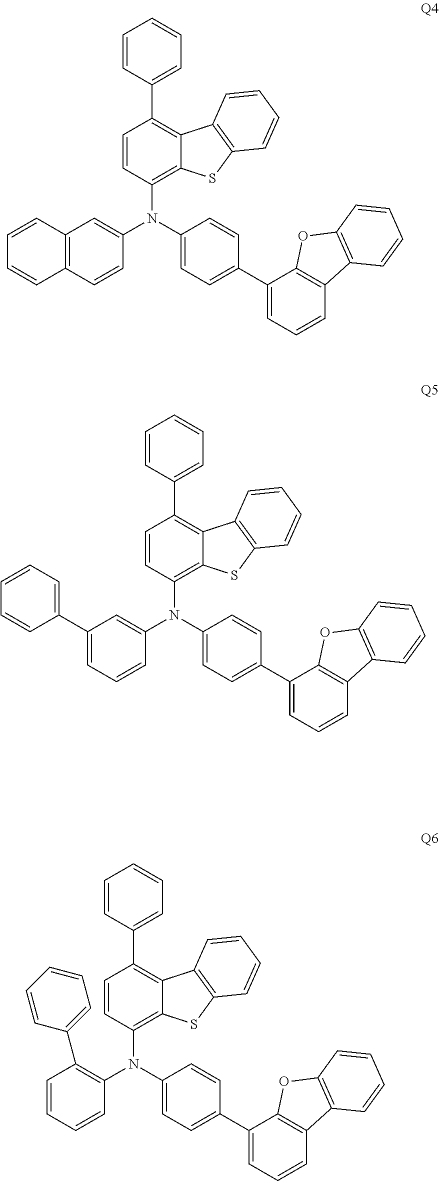 [SMILES: c1ccc(-c2ccc(N(c3ccc(-c4cccc5c4oc4ccccc45)cc3)c3ccc4ccccc4c3)c3sc4ccccc4c23)cc1 ; c1ccc(-c2cccc(N(c3ccc(-c4cccc5c4oc4ccccc45)cc3)c3ccc(-c4ccccc4)c4c3sc3ccccc34)c2)cc1 ; c1ccc(-c2ccccc2N(c2ccc(-c3cccc4c3oc3ccccc34)cc2)c2ccc(-c3ccccc3)c3c2sc2ccccc23)cc1]

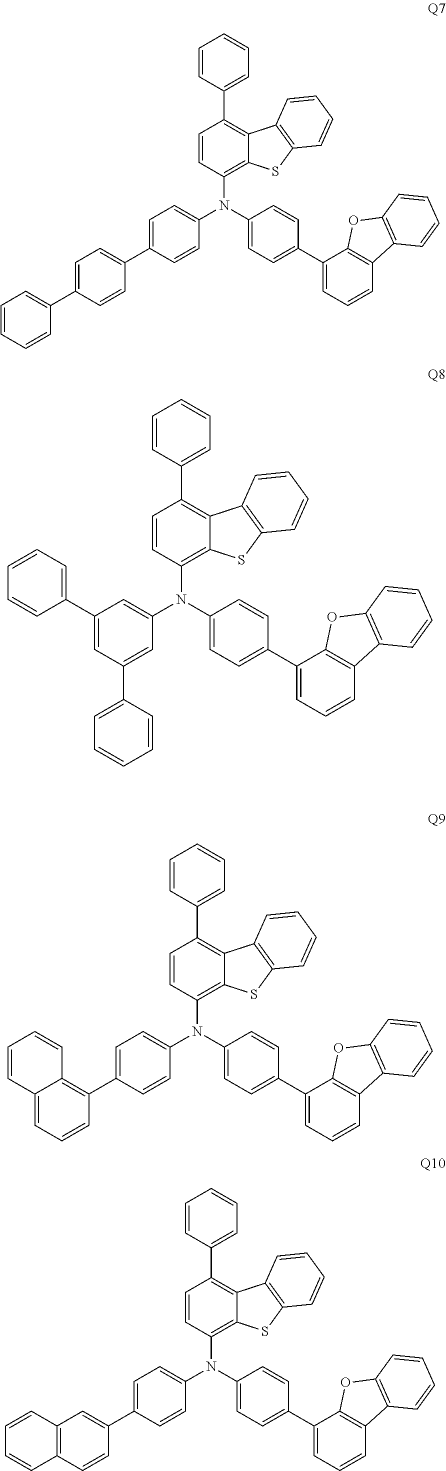 [SMILES: c1ccc(-c2ccc(-c3ccc(N(c4ccc(-c5cccc6c5oc5ccccc56)cc4)c4ccc(-c5ccccc5)c5c4sc4ccccc45)cc3)cc2)cc1 ; c1ccc(-c2cc(-c3ccccc3)cc(N(c3ccc(-c4cccc5c4oc4ccccc45)cc3)c3ccc(-c4ccccc4)c4c3sc3ccccc34)c2)cc1 ; c1ccc(-c2ccc(N(c3ccc(-c4cccc5ccccc45)cc3)c3ccc(-c4cccc5c4oc4ccccc45)cc3)c3sc4ccccc4c23)cc1 ; c1ccc(-c2ccc(N(c3ccc(-c4ccc5ccccc5c4)cc3)c3ccc(-c4cccc5c4oc4ccccc45)cc3)c3sc4ccccc4c23)cc1]

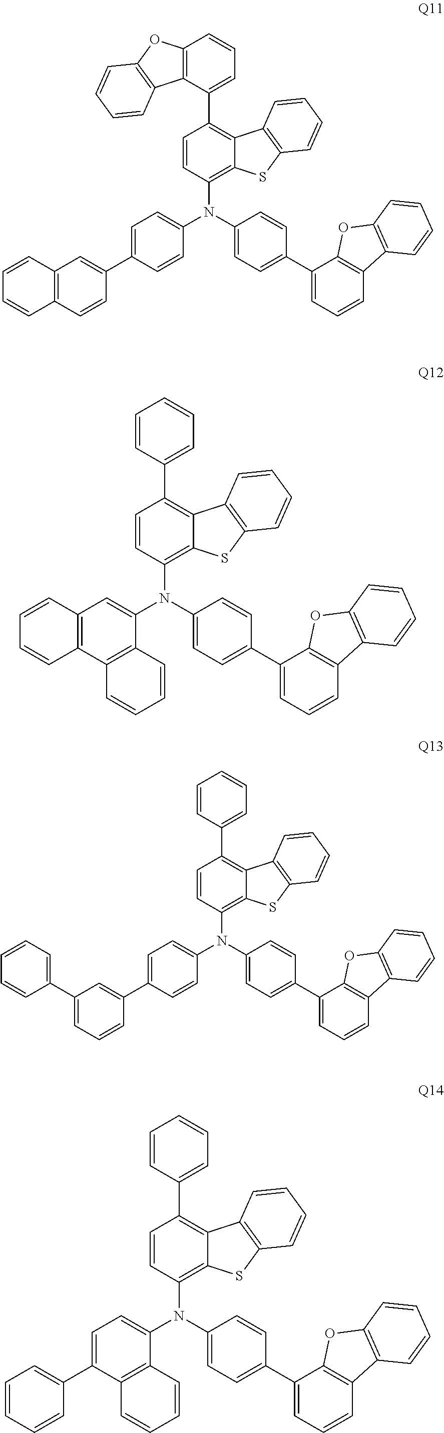 [SMILES: c1ccc2cc(-c3ccc(N(c4ccc(-c5cccc6c5oc5ccccc56)cc4)c4ccc(-c5cccc6oc7ccccc7c56)c5c4sc4ccccc45)cc3)ccc2c1 ; c1ccc(-c2ccc(N(c3ccc(-c4cccc5c4oc4ccccc45)cc3)c3cc4ccccc4c4ccccc34)c3sc4ccccc4c23)cc1 ; c1ccc(-c2cccc(-c3ccc(N(c4ccc(-c5cccc6c5oc5ccccc56)cc4)c4ccc(-c5ccccc5)c5c4sc4ccccc45)cc3)c2)cc1 ; c1ccc(-c2ccc(N(c3ccc(-c4cccc5c4oc4ccccc45)cc3)c3ccc(-c4ccccc4)c4c3sc3ccccc34)c3ccccc23)cc1]

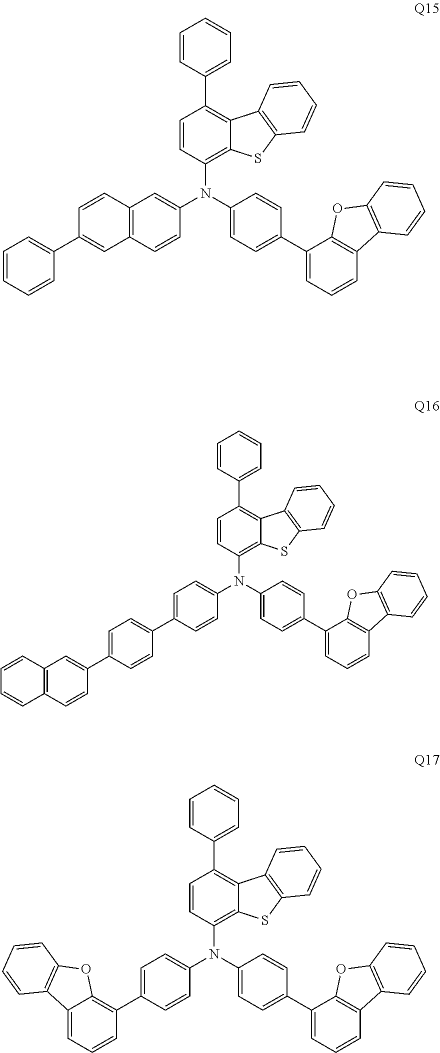 [SMILES: c1ccc(-c2ccc3cc(N(c4ccc(-c5cccc6c5oc5ccccc56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4cccc5c4oc4ccccc45)cc3)c3sc4ccccc4c23)cc1 ; c1ccc(-c2ccc(N(c3ccc(-c4cccc5c4oc4ccccc45)cc3)c3ccc(-c4cccc5c4oc4ccccc45)cc3)c3sc4ccccc4c23)cc1]

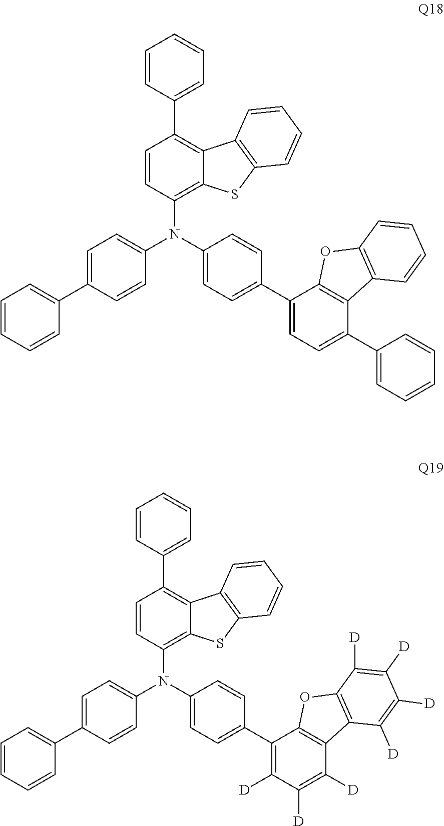 [SMILES: c1ccc(-c2ccc(N(c3ccc(-c4ccc(-c5ccccc5)c5c4oc4ccccc45)cc3)c3ccc(-c4ccccc4)c4c3sc3ccccc34)cc2)cc1 ; [2H]c1c([2H])c([2H])c2c(oc3c(-c4ccc(N(c5ccc(-c6ccccc6)cc5)c5ccc(-c6ccccc6)c6c5sc5ccccc56)cc4)c([2H])c([2H])c([2H])c32)c1[2H]]

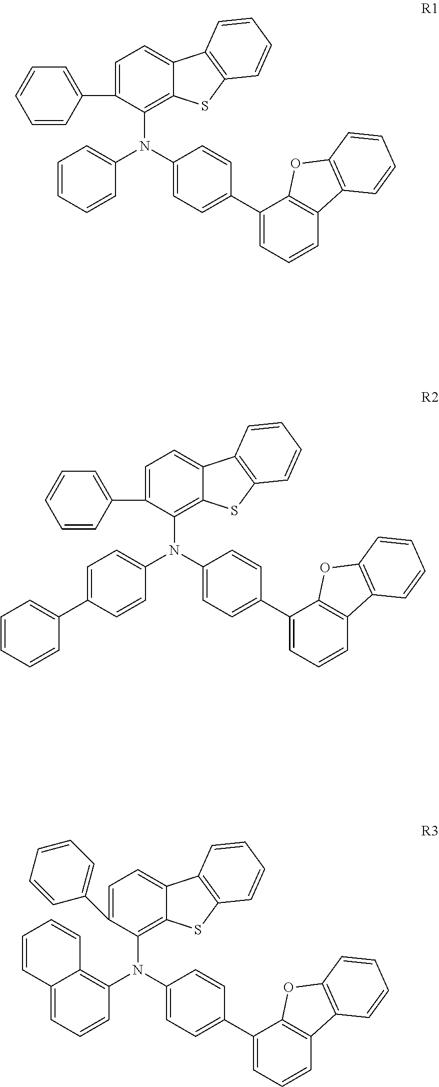 [SMILES: c1ccc(-c2ccc3c(sc4ccccc43)c2N(c2ccccc2)c2ccc(-c3cccc4c3oc3ccccc34)cc2)cc1 ; c1ccc(-c2ccc(N(c3ccc(-c4cccc5c4oc4ccccc45)cc3)c3c(-c4ccccc4)ccc4c3sc3ccccc34)cc2)cc1 ; c1ccc(-c2ccc3c(sc4ccccc43)c2N(c2ccc(-c3cccc4c3oc3ccccc34)cc2)c2cccc3ccccc23)cc1]

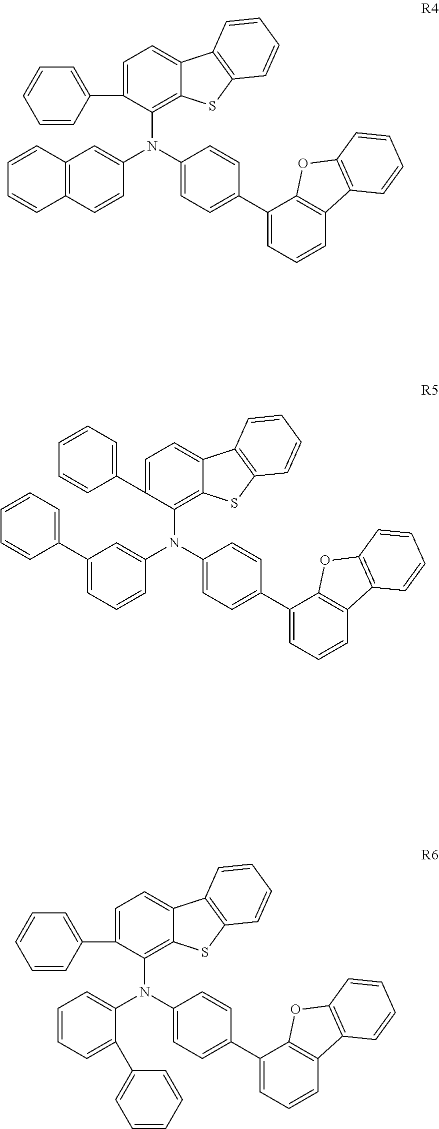 [SMILES: c1ccc(-c2ccc3c(sc4ccccc43)c2N(c2ccc(-c3cccc4c3oc3ccccc34)cc2)c2ccc3ccccc3c2)cc1 ; c1ccc(-c2cccc(N(c3ccc(-c4cccc5c4oc4ccccc45)cc3)c3c(-c4ccccc4)ccc4c3sc3ccccc34)c2)cc1 ; c1ccc(-c2ccccc2N(c2ccc(-c3cccc4c3oc3ccccc34)cc2)c2c(-c3ccccc3)ccc3c2sc2ccccc23)cc1]

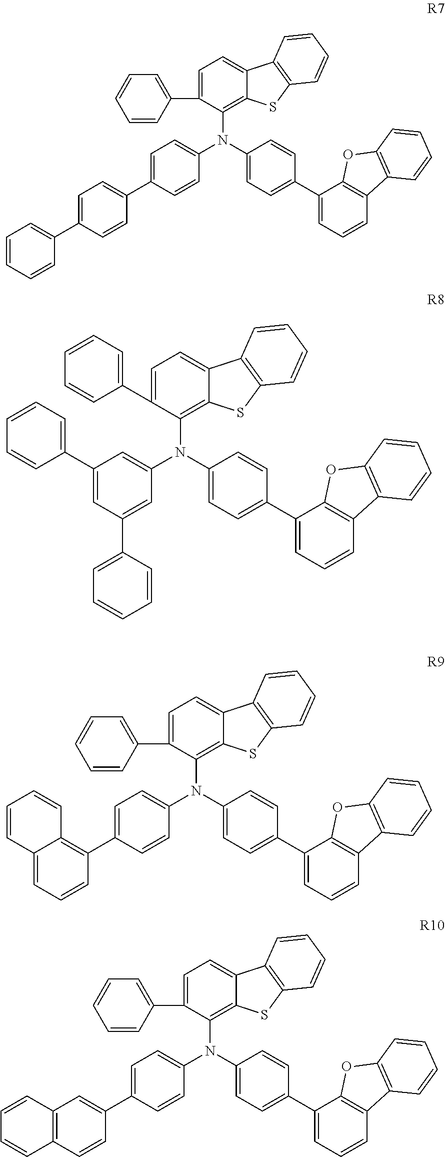

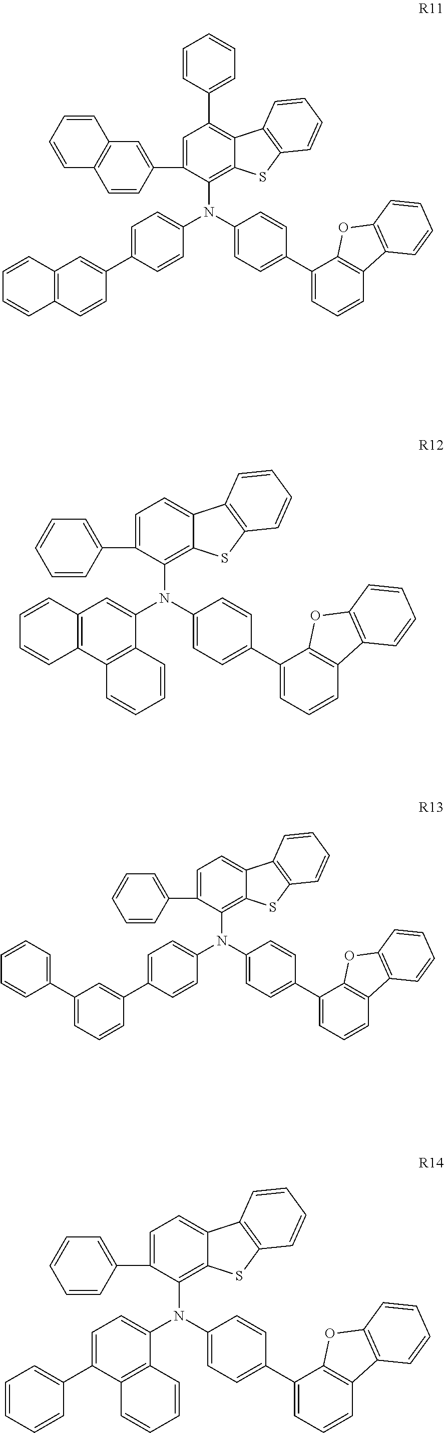 [SMILES: c1ccc(-c2cc(-c3ccc4ccccc4c3)c(N(c3ccc(-c4ccc5ccccc5c4)cc3)c3ccc(-c4cccc5c4oc4ccccc45)cc3)c3sc4ccccc4c23)cc1 ; c1ccc(-c2ccc3c(sc4ccccc43)c2N(c2ccc(-c3cccc4c3oc3ccccc34)cc2)c2cc3ccccc3c3ccccc23)cc1 ; c1ccc(-c2cccc(-c3ccc(N(c4ccc(-c5cccc6c5oc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3cccc4c3oc3ccccc34)cc2)c2ccc(-c3ccccc3)c3ccccc23)cc1]

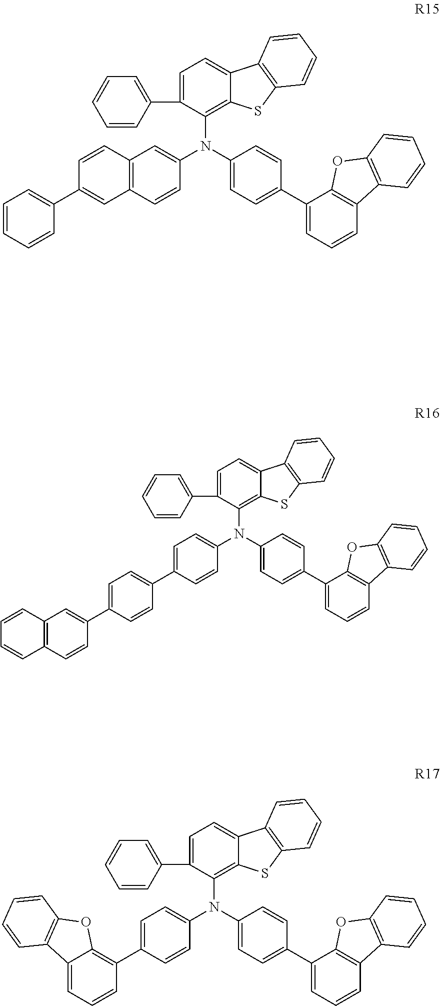 [SMILES: c1ccc(-c2ccc3cc(N(c4ccc(-c5cccc6c5oc5ccccc56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3cccc4c3oc3ccccc34)cc2)cc1 ; c1ccc(-c2ccc3c(sc4ccccc43)c2N(c2ccc(-c3cccc4c3oc3ccccc34)cc2)c2ccc(-c3cccc4c3oc3ccccc34)cc2)cc1]

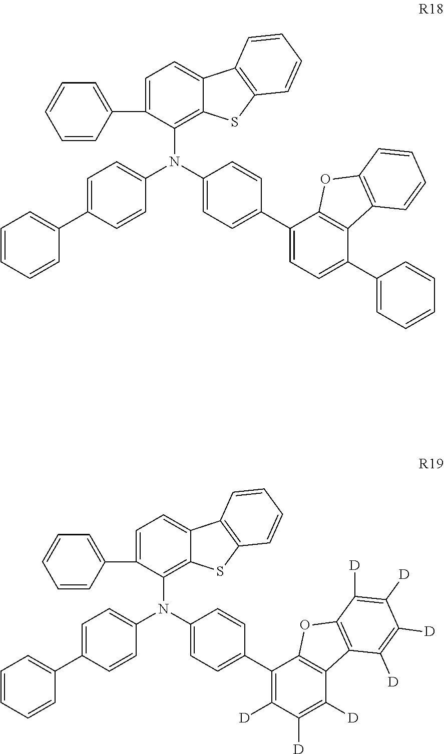 [SMILES: c1ccc(-c2ccc(N(c3ccc(-c4ccc(-c5ccccc5)c5c4oc4ccccc45)cc3)c3c(-c4ccccc4)ccc4c3sc3ccccc34)cc2)cc1 ; [2H]c1c([2H])c([2H])c2c(oc3c(-c4ccc(N(c5ccc(-c6ccccc6)cc5)c5c(-c6ccccc6)ccc6c5sc5ccccc56)cc4)c([2H])c([2H])c([2H])c32)c1[2H]]

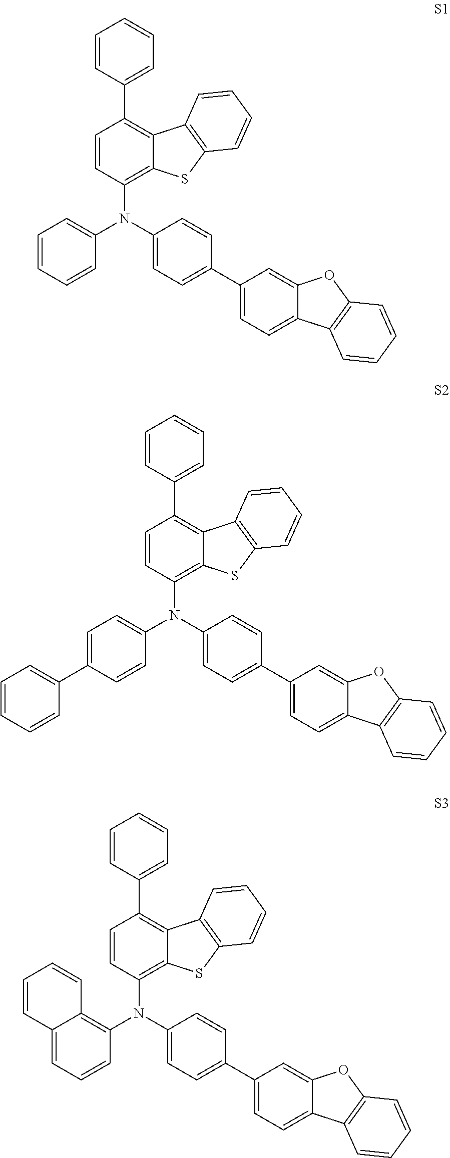 [SMILES: c1ccc(-c2ccc(N(c3ccccc3)c3ccc(-c4ccc5c(c4)oc4ccccc45)cc3)c3sc4ccccc4c23)cc1 ; c1ccc(-c2ccc(N(c3ccc(-c4ccc5c(c4)oc4ccccc45)cc3)c3ccc(-c4ccccc4)c4c3sc3ccccc34)cc2)cc1 ; c1ccc(-c2ccc(N(c3ccc(-c4ccc5c(c4)oc4ccccc45)cc3)c3cccc4ccccc34)c3sc4ccccc4c23)cc1]

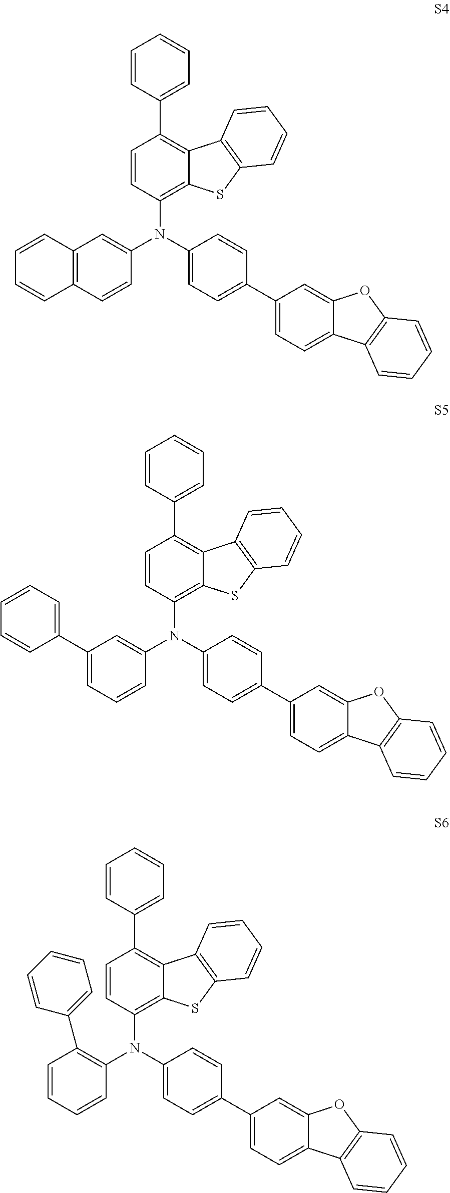 [SMILES: c1ccc(-c2ccc(N(c3ccc(-c4ccc5c(c4)oc4ccccc45)cc3)c3ccc4ccccc4c3)c3sc4ccccc4c23)cc1 ; c1ccc(-c2cccc(N(c3ccc(-c4ccc5c(c4)oc4ccccc45)cc3)c3ccc(-c4ccccc4)c4c3sc3ccccc34)c2)cc1 ; c1ccc(-c2ccccc2N(c2ccc(-c3ccc4c(c3)oc3ccccc34)cc2)c2ccc(-c3ccccc3)c3c2sc2ccccc23)cc1]

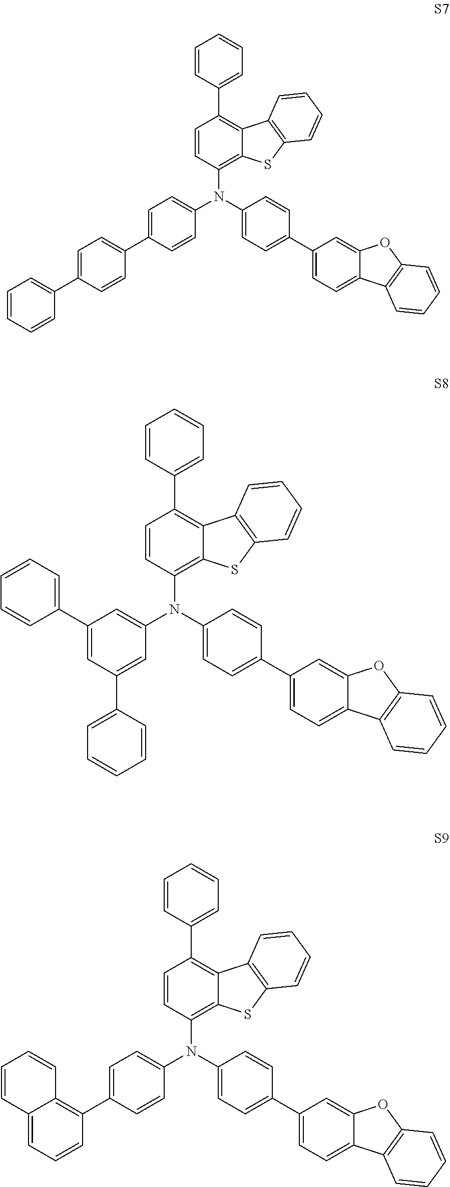 [SMILES: c1ccc(-c2ccc(-c3ccc(N(c4ccc(-c5ccc6c(c5)oc5ccccc56)cc4)c4ccc(-c5ccccc5)c5c4sc4ccccc45)cc3)cc2)cc1 ; c1ccc(-c2cc(-c3ccccc3)cc(N(c3ccc(-c4ccc5c(c4)oc4ccccc45)cc3)c3ccc(-c4ccccc4)c4c3sc3ccccc34)c2)cc1 ; c1ccc(-c2ccc(N(c3ccc(-c4ccc5c(c4)oc4ccccc45)cc3)c3ccc(-c4cccc5ccccc45)cc3)c3sc4ccccc4c23)cc1]

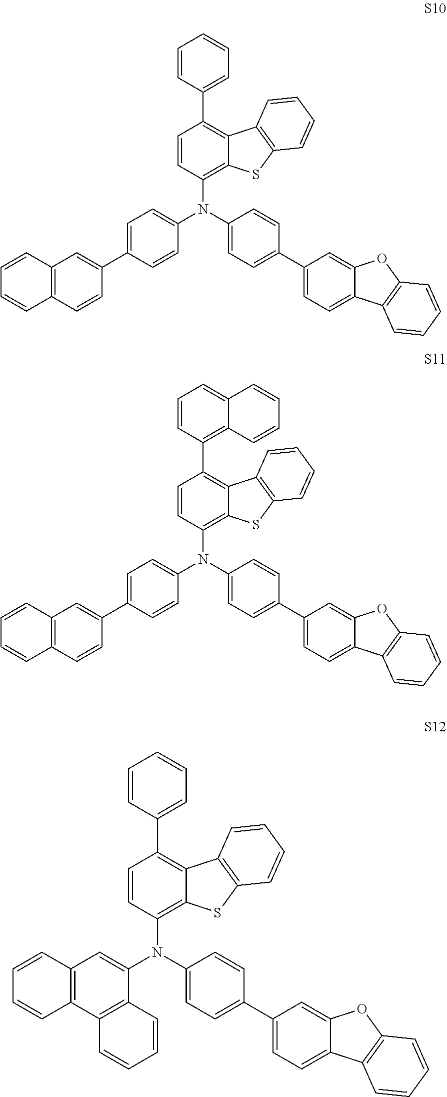 [SMILES: c1ccc(-c2ccc(N(c3ccc(-c4ccc5ccccc5c4)cc3)c3ccc(-c4ccc5c(c4)oc4ccccc45)cc3)c3sc4ccccc4c23)cc1 ; c1ccc2cc(-c3ccc(N(c4ccc(-c5ccc6c(c5)oc5ccccc56)cc4)c4ccc(-c5cccc6ccccc56)c5c4sc4ccccc45)cc3)ccc2c1 ; c1ccc(-c2ccc(N(c3ccc(-c4ccc5c(c4)oc4ccccc45)cc3)c3cc4ccccc4c4ccccc34)c3sc4ccccc4c23)cc1]

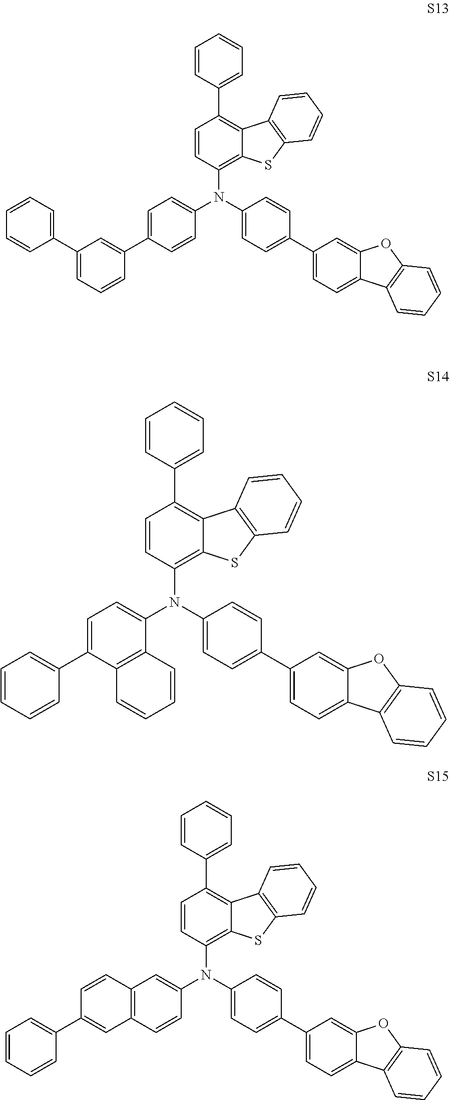 [SMILES: c1ccc(-c2cccc(-c3ccc(N(c4ccc(-c5ccc6c(c5)oc5ccccc56)cc4)c4ccc(-c5ccccc5)c5c4sc4ccccc45)cc3)c2)cc1 ; c1ccc(-c2ccc(N(c3ccc(-c4ccc5c(c4)oc4ccccc45)cc3)c3ccc(-c4ccccc4)c4c3sc3ccccc34)c3ccccc23)cc1 ; c1ccc(-c2ccc3cc(N(c4ccc(-c5ccc6c(c5)oc5ccccc56)cc4)c4ccc(-c5ccccc5)c5c4sc4ccccc45)ccc3c2)cc1]

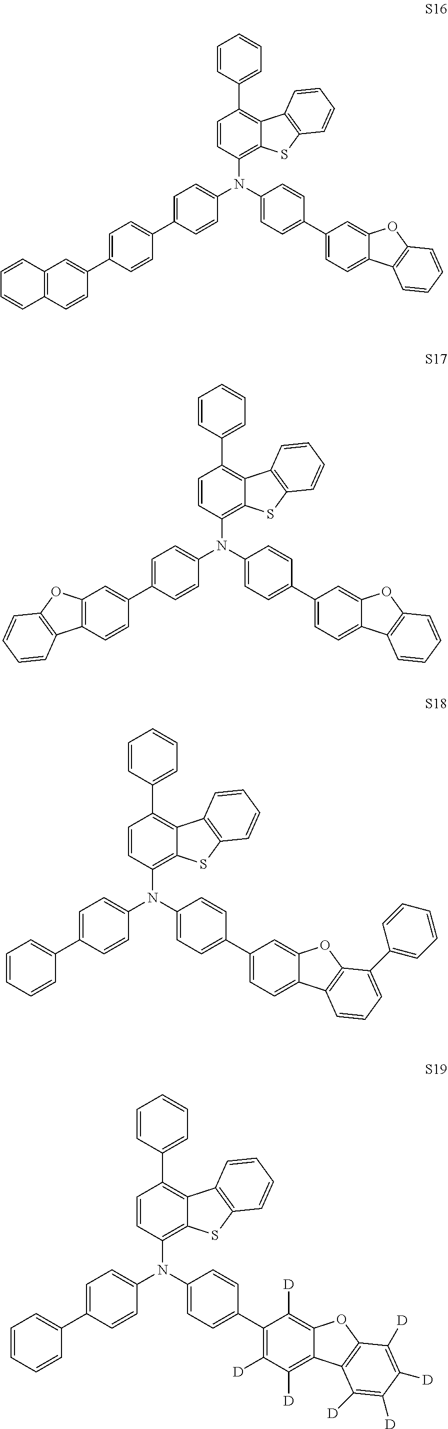 [SMILES: c1ccc(-c2ccc(N(c3ccc(-c4ccc(-c5ccc6ccccc6c5)cc4)cc3)c3ccc(-c4ccc5c(c4)oc4ccccc45)cc3)c3sc4ccccc4c23)cc1 ; c1ccc(-c2ccc(N(c3ccc(-c4ccc5c(c4)oc4ccccc45)cc3)c3ccc(-c4ccc5c(c4)oc4ccccc45)cc3)c3sc4ccccc4c23)cc1 ; c1ccc(-c2ccc(N(c3ccc(-c4ccc5c(c4)oc4c(-c6ccccc6)cccc45)cc3)c3ccc(-c4ccccc4)c4c3sc3ccccc34)cc2)cc1 ; [2H]c1c([2H])c([2H])c2c(oc3c([2H])c(-c4ccc(N(c5ccc(-c6ccccc6)cc5)c5ccc(-c6ccccc6)c6c5sc5ccccc56)cc4)c([2H])c([2H])c32)c1[2H]]

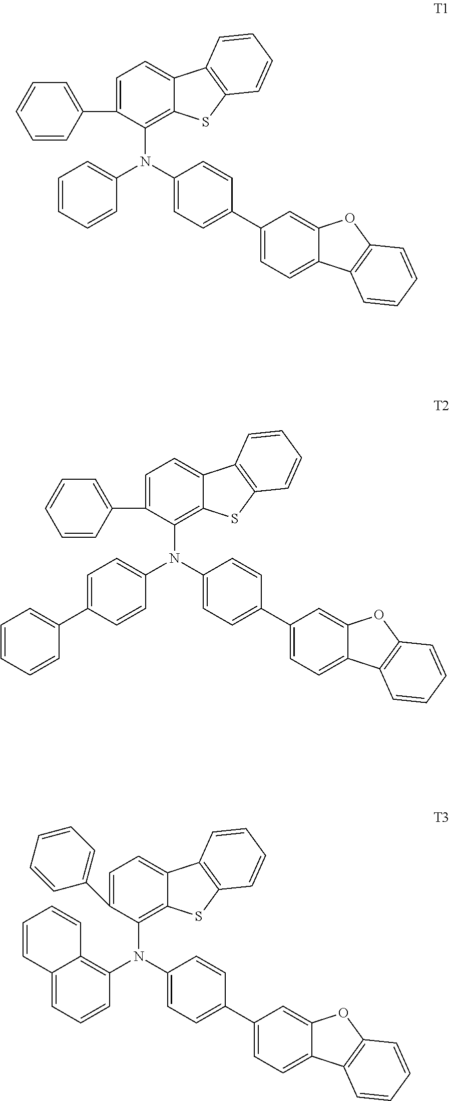 [SMILES: c1ccc(-c2ccc3c(sc4ccccc43)c2N(c2ccccc2)c2ccc(-c3ccc4c(c3)oc3ccccc34)cc2)cc1 ; c1ccc(-c2ccc(N(c3ccc(-c4ccc5c(c4)oc4ccccc45)cc3)c3c(-c4ccccc4)ccc4c3sc3ccccc34)cc2)cc1 ; c1ccc(-c2ccc3c(sc4ccccc43)c2N(c2ccc(-c3ccc4c(c3)oc3ccccc34)cc2)c2cccc3ccccc23)cc1]

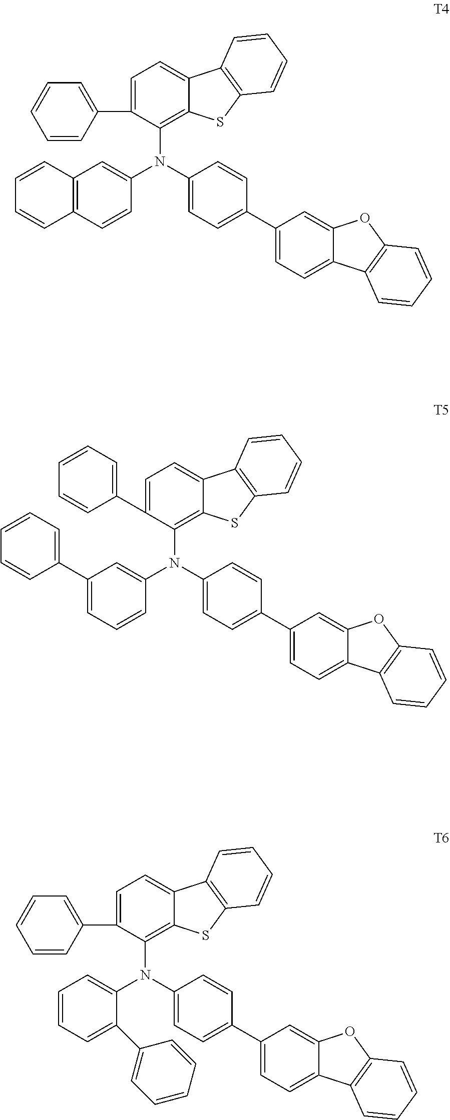 [SMILES: c1ccc(-c2ccc3c(sc4ccccc43)c2N(c2ccc(-c3ccc4c(c3)oc3ccccc34)cc2)c2ccc3ccccc3c2)cc1 ; c1ccc(-c2cccc(N(c3ccc(-c4ccc5c(c4)oc4ccccc45)cc3)c3c(-c4ccccc4)ccc4c3sc3ccccc34)c2)cc1 ; c1ccc(-c2ccccc2N(c2ccc(-c3ccc4c(c3)oc3ccccc34)cc2)c2c(-c3ccccc3)ccc3c2sc2ccccc23)cc1]

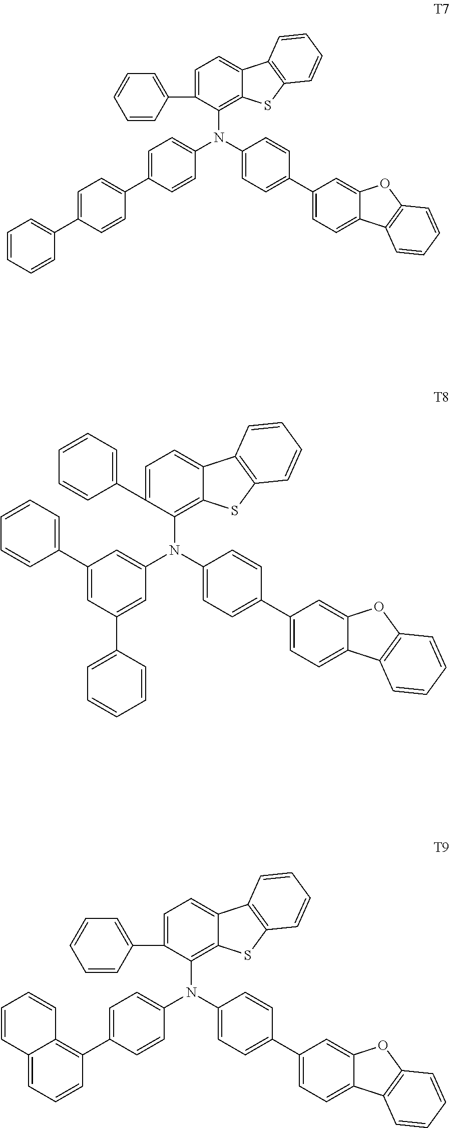 [SMILES: c1ccc(-c2ccc(-c3ccc(N(c4ccc(-c5ccc6c(c5)oc5ccccc56)cc4)c4c(-c5ccccc5)ccc5c4sc4ccccc45)cc3)cc2)cc1 ; c1ccc(-c2cc(-c3ccccc3)cc(N(c3ccc(-c4ccc5c(c4)oc4ccccc45)cc3)c3c(-c4ccccc4)ccc4c3sc3ccccc34)c2)cc1 ; c1ccc(-c2ccc3c(sc4ccccc43)c2N(c2ccc(-c3ccc4c(c3)oc3ccccc34)cc2)c2ccc(-c3cccc4ccccc34)cc2)cc1]

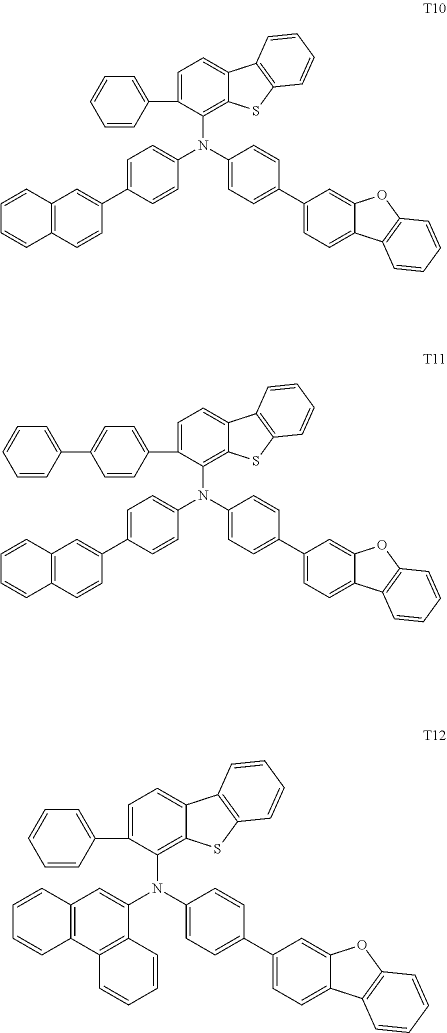 [SMILES: c1ccc(-c2ccc3c(sc4ccccc43)c2N(c2ccc(-c3ccc4ccccc4c3)cc2)c2ccc(-c3ccc4c(c3)oc3ccccc34)cc2)cc1 ; c1ccc(-c2ccc(-c3ccc4c(sc5ccccc54)c3N(c3ccc(-c4ccc5ccccc5c4)cc3)c3ccc(-c4ccc5c(c4)oc4ccccc45)cc3)cc2)cc1 ; c1ccc(-c2ccc3c(sc4ccccc43)c2N(c2ccc(-c3ccc4c(c3)oc3ccccc34)cc2)c2cc3ccccc3c3ccccc23)cc1]

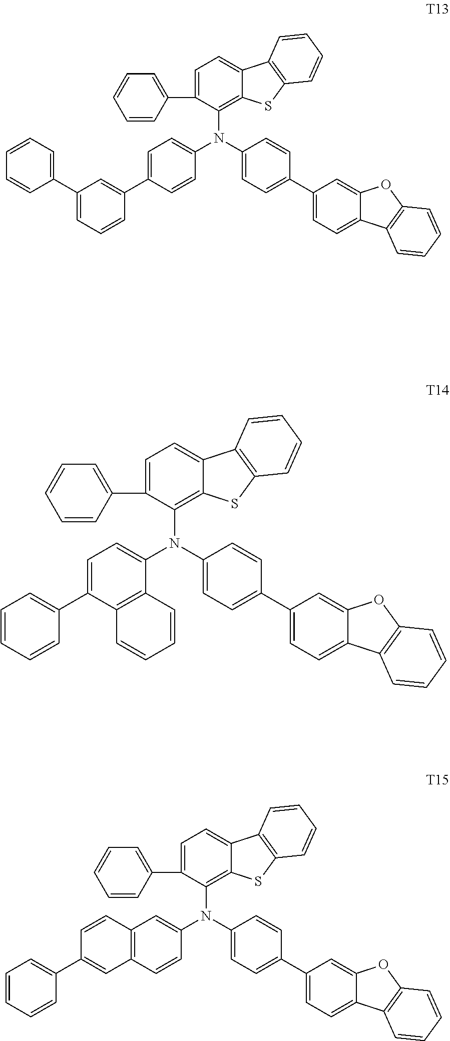 [SMILES: c1ccc(-c2cccc(-c3ccc(N(c4ccc(-c5ccc6c(c5)oc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3ccc4c(c3)oc3ccccc34)cc2)c2ccc(-c3ccccc3)c3ccccc23)cc1 ; c1ccc(-c2ccc3cc(N(c4ccc(-c5ccc6c(c5)oc5ccccc56)cc4)c4c(-c5ccccc5)ccc5c4sc4ccccc45)ccc3c2)cc1]

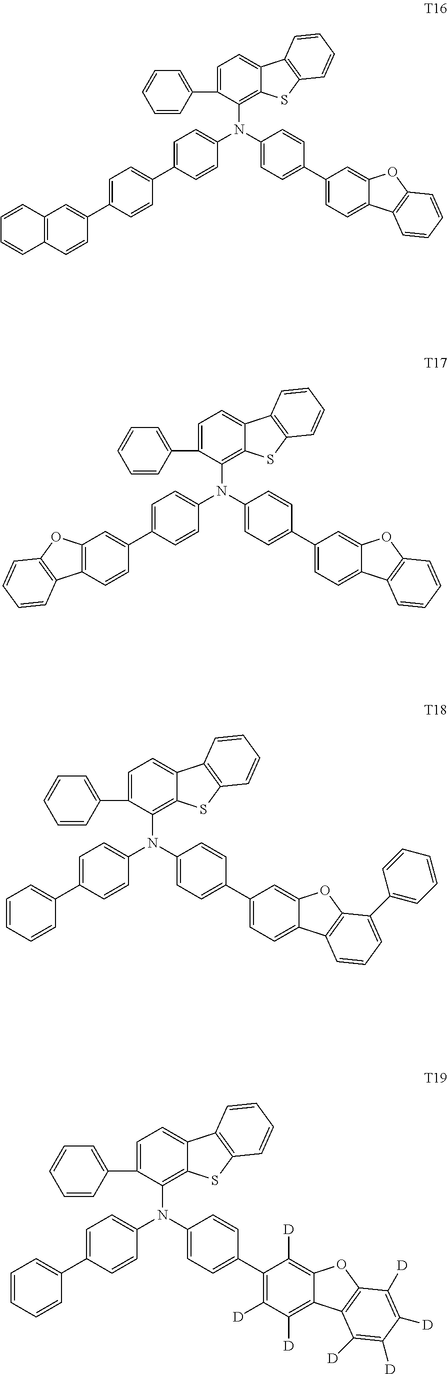 [SMILES: c1ccc(-c2ccc3c(sc4ccccc43)c2N(c2ccc(-c3ccc(-c4ccc5ccccc5c4)cc3)cc2)c2ccc(-c3ccc4c(c3)oc3ccccc34)cc2)cc1 ; c1ccc(-c2ccc3c(sc4ccccc43)c2N(c2ccc(-c3ccc4c(c3)oc3ccccc34)cc2)c2ccc(-c3ccc4c(c3)oc3ccccc34)cc2)cc1 ; c1ccc(-c2ccc(N(c3ccc(-c4ccc5c(c4)oc4c(-c6ccccc6)cccc45)cc3)c3c(-c4ccccc4)ccc4c3sc3ccccc34)cc2)cc1 ; [2H]c1c([2H])c([2H])c2c(oc3c([2H])c(-c4ccc(N(c5ccc(-c6ccccc6)cc5)c5c(-c6ccccc6)ccc6c5sc5ccccc56)cc4)c([2H])c([2H])c32)c1[2H]]

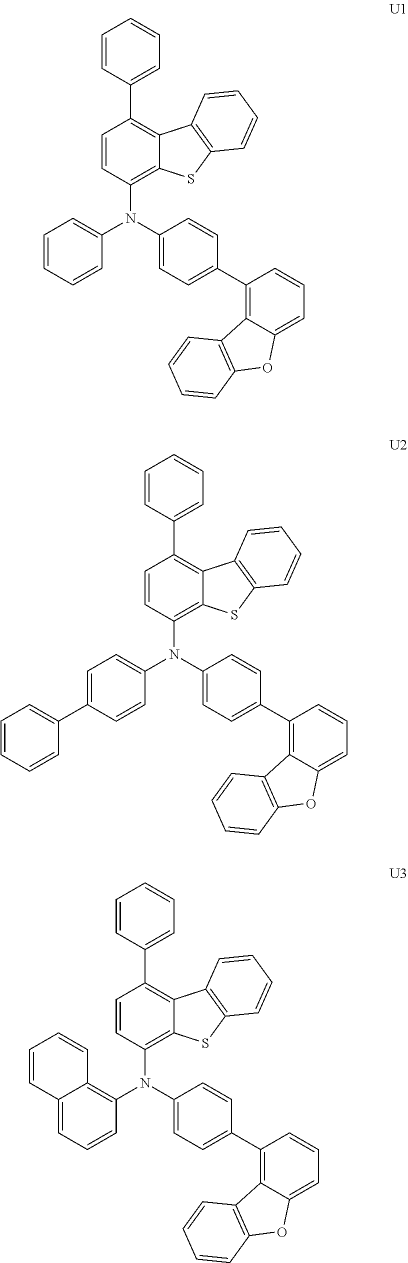 [SMILES: c1ccc(-c2ccc(N(c3ccccc3)c3ccc(-c4cccc5oc6ccccc6c45)cc3)c3sc4ccccc4c23)cc1 ; c1ccc(-c2ccc(N(c3ccc(-c4cccc5oc6ccccc6c45)cc3)c3ccc(-c4ccccc4)c4c3sc3ccccc34)cc2)cc1 ; c1ccc(-c2ccc(N(c3ccc(-c4cccc5oc6ccccc6c45)cc3)c3cccc4ccccc34)c3sc4ccccc4c23)cc1]

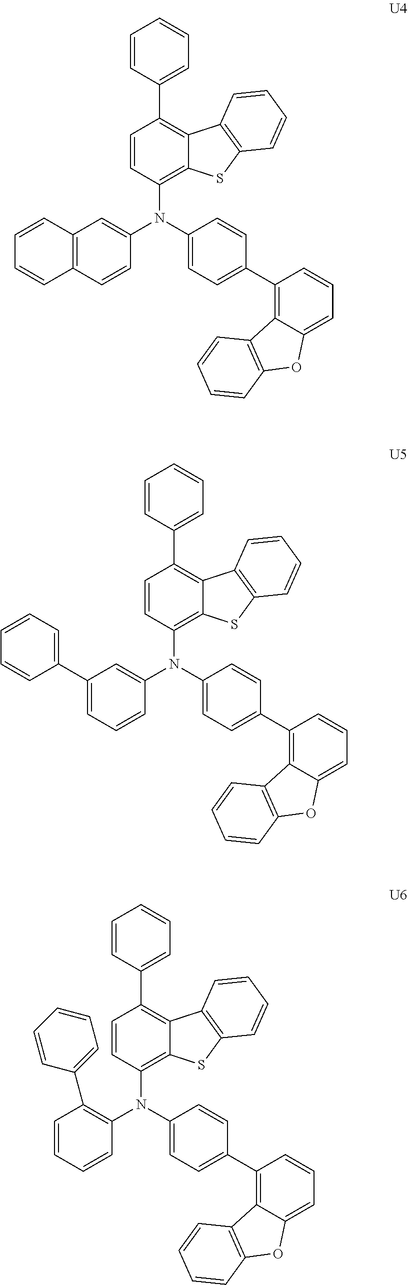 [SMILES: c1ccc(-c2ccc(N(c3ccc(-c4cccc5oc6ccccc6c45)cc3)c3ccc4ccccc4c3)c3sc4ccccc4c23)cc1 ; c1ccc(-c2cccc(N(c3ccc(-c4cccc5oc6ccccc6c45)cc3)c3ccc(-c4ccccc4)c4c3sc3ccccc34)c2)cc1 ; c1ccc(-c2ccccc2N(c2ccc(-c3cccc4oc5ccccc5c34)cc2)c2ccc(-c3ccccc3)c3c2sc2ccccc23)cc1]

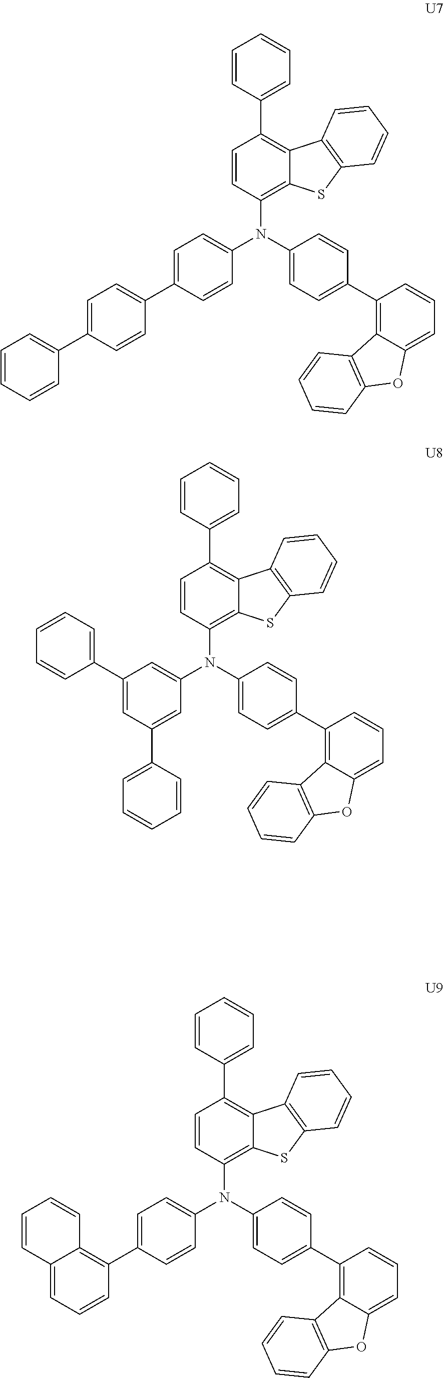 [SMILES: c1ccc(-c2ccc(-c3ccc(N(c4ccc(-c5cccc6oc7ccccc7c56)cc4)c4ccc(-c5ccccc5)c5c4sc4ccccc45)cc3)cc2)cc1 ; c1ccc(-c2cc(-c3ccccc3)cc(N(c3ccc(-c4cccc5oc6ccccc6c45)cc3)c3ccc(-c4ccccc4)c4c3sc3ccccc34)c2)cc1 ; c1ccc(-c2ccc(N(c3ccc(-c4cccc5ccccc45)cc3)c3ccc(-c4cccc5oc6ccccc6c45)cc3)c3sc4ccccc4c23)cc1]

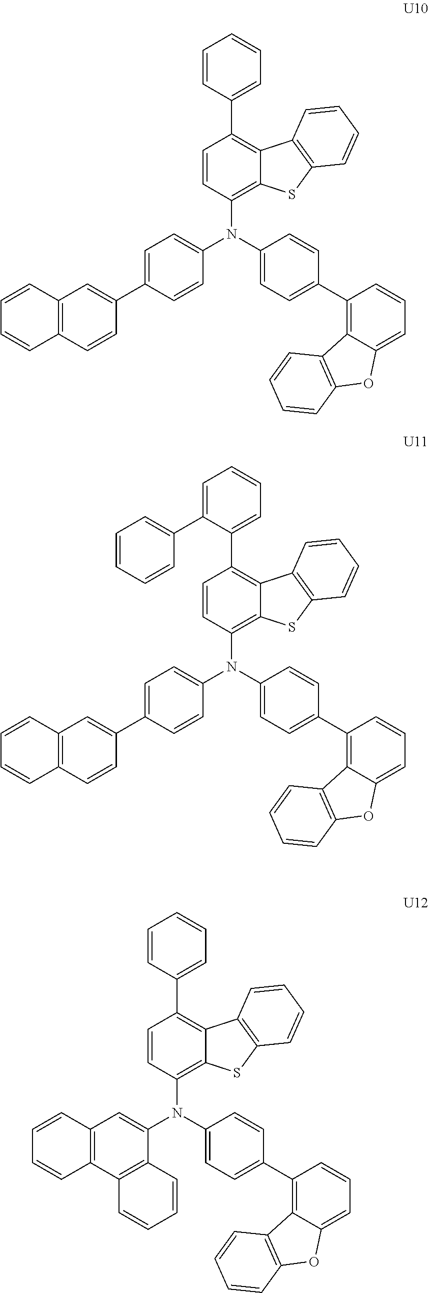 [SMILES: c1ccc(-c2ccc(N(c3ccc(-c4ccc5ccccc5c4)cc3)c3ccc(-c4cccc5oc6ccccc6c45)cc3)c3sc4ccccc4c23)cc1 ; c1ccc(-c2ccccc2-c2ccc(N(c3ccc(-c4ccc5ccccc5c4)cc3)c3ccc(-c4cccc5oc6ccccc6c45)cc3)c3sc4ccccc4c23)cc1 ; c1ccc(-c2ccc(N(c3ccc(-c4cccc5oc6ccccc6c45)cc3)c3cc4ccccc4c4ccccc34)c3sc4ccccc4c23)cc1]

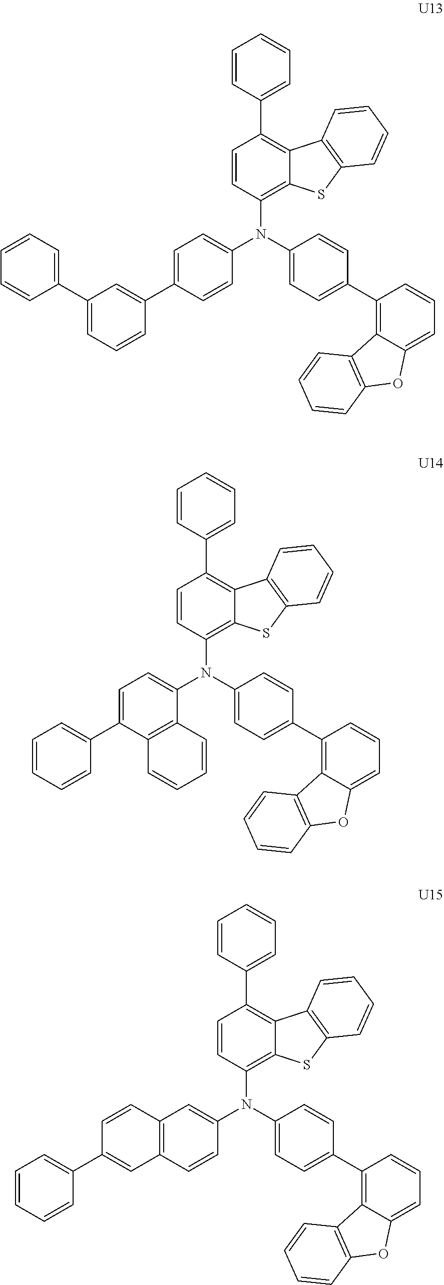 [SMILES: c1ccc(-c2cccc(-c3ccc(N(c4ccc(-c5cccc6oc7ccccc7c56)cc4)c4ccc(-c5ccccc5)c5c4sc4ccccc45)cc3)c2)cc1 ; c1ccc(-c2ccc(N(c3ccc(-c4cccc5oc6ccccc6c45)cc3)c3ccc(-c4ccccc4)c4c3sc3ccccc34)c3ccccc23)cc1 ; c1ccc(-c2ccc3cc(N(c4ccc(-c5cccc6oc7ccccc7c56)cc4)c4ccc(-c5ccccc5)c5c4sc4ccccc45)ccc3c2)cc1]

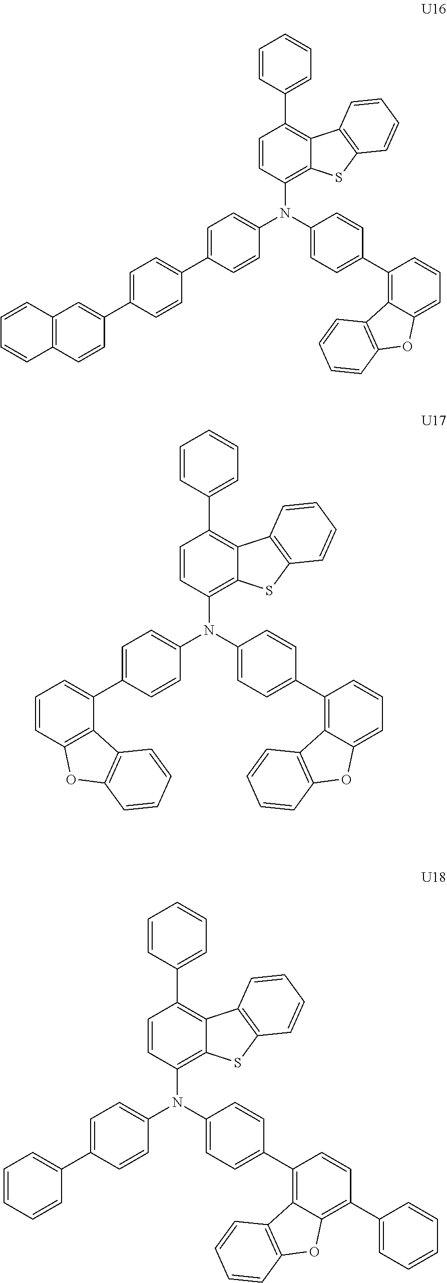 [SMILES: c1ccc(-c2ccc(N(c3ccc(-c4ccc(-c5ccc6ccccc6c5)cc4)cc3)c3ccc(-c4cccc5oc6ccccc6c45)cc3)c3sc4ccccc4c23)cc1 ; c1ccc(-c2ccc(N(c3ccc(-c4cccc5oc6ccccc6c45)cc3)c3ccc(-c4cccc5oc6ccccc6c45)cc3)c3sc4ccccc4c23)cc1 ; c1ccc(-c2ccc(N(c3ccc(-c4ccc(-c5ccccc5)c5oc6ccccc6c45)cc3)c3ccc(-c4ccccc4)c4c3sc3ccccc34)cc2)cc1]

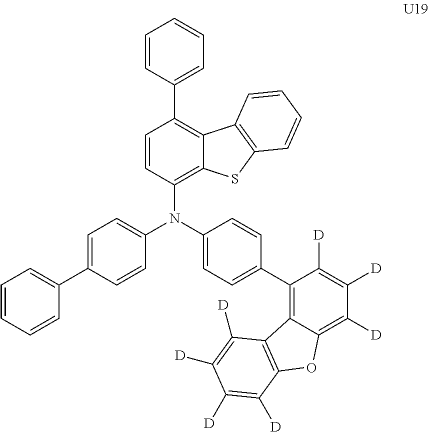 [SMILES: [2H]c1c([2H])c([2H])c2c(oc3c([2H])c([2H])c([2H])c(-c4ccc(N(c5ccc(-c6ccccc6)cc5)c5ccc(-c6ccccc6)c6c5sc5ccccc56)cc4)c32)c1[2H]]

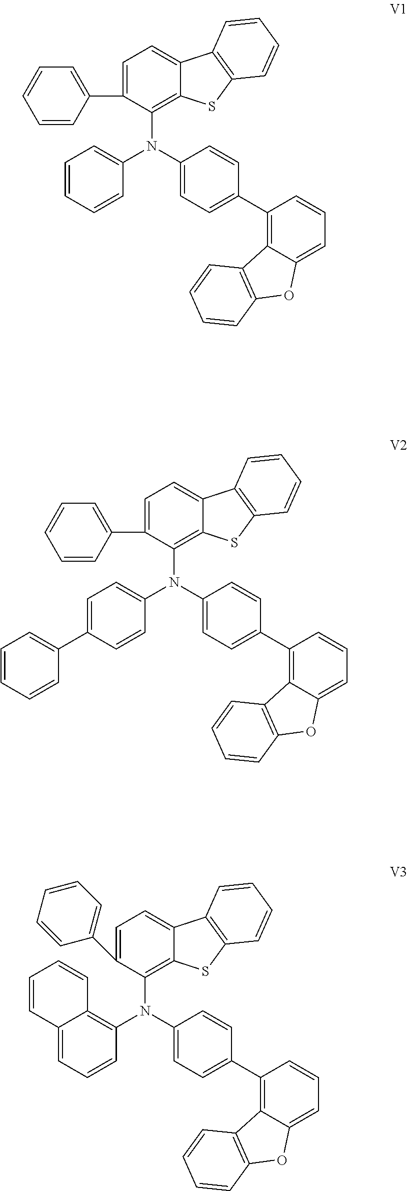 [SMILES: c1ccc(-c2ccc3c(sc4ccccc43)c2N(c2ccccc2)c2ccc(-c3cccc4oc5ccccc5c34)cc2)cc1 ; c1ccc(-c2ccc(N(c3ccc(-c4cccc5oc6ccccc6c45)cc3)c3c(-c4ccccc4)ccc4c3sc3ccccc34)cc2)cc1 ; c1ccc(-c2ccc3c(sc4ccccc43)c2N(c2ccc(-c3cccc4oc5ccccc5c34)cc2)c2cccc3ccccc23)cc1]

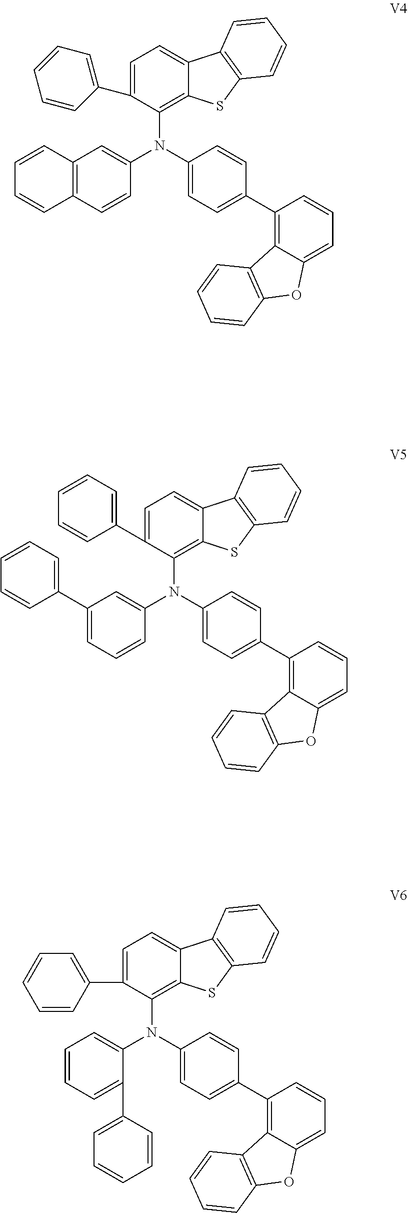 [SMILES: c1ccc(-c2ccc3c(sc4ccccc43)c2N(c2ccc(-c3cccc4oc5ccccc5c34)cc2)c2ccc3ccccc3c2)cc1 ; c1ccc(-c2cccc(N(c3ccc(-c4cccc5oc6ccccc6c45)cc3)c3c(-c4ccccc4)ccc4c3sc3ccccc34)c2)cc1 ; c1ccc(-c2ccccc2N(c2ccc(-c3cccc4oc5ccccc5c34)cc2)c2c(-c3ccccc3)ccc3c2sc2ccccc23)cc1]

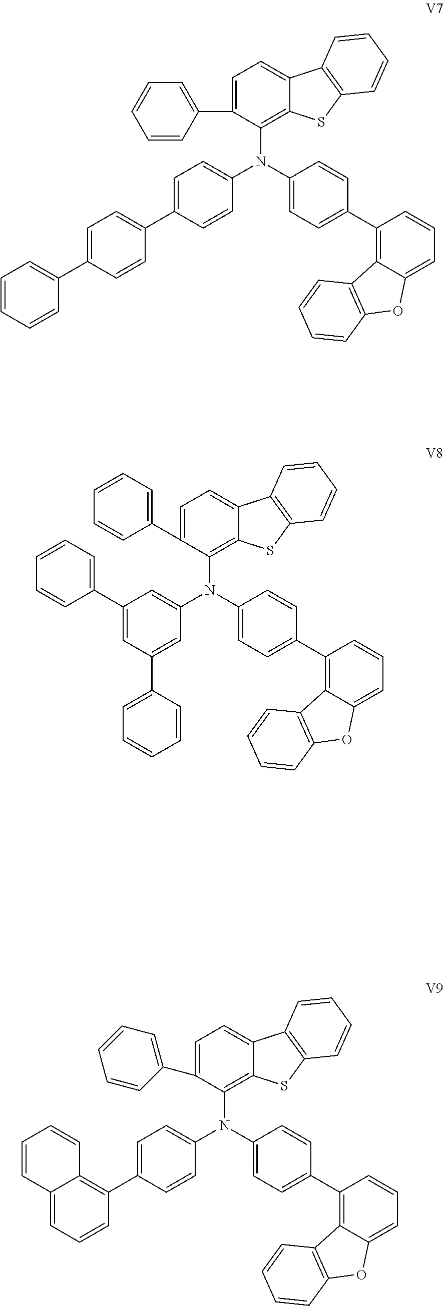 [SMILES: c1ccc(-c2ccc(-c3ccc(N(c4ccc(-c5cccc6oc7ccccc7c56)cc4)c4c(-c5ccccc5)ccc5c4sc4ccccc45)cc3)cc2)cc1 ; c1ccc(-c2cc(-c3ccccc3)cc(N(c3ccc(-c4cccc5oc6ccccc6c45)cc3)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2ccc(-c3cccc4oc5ccccc5c34)cc2)cc1]

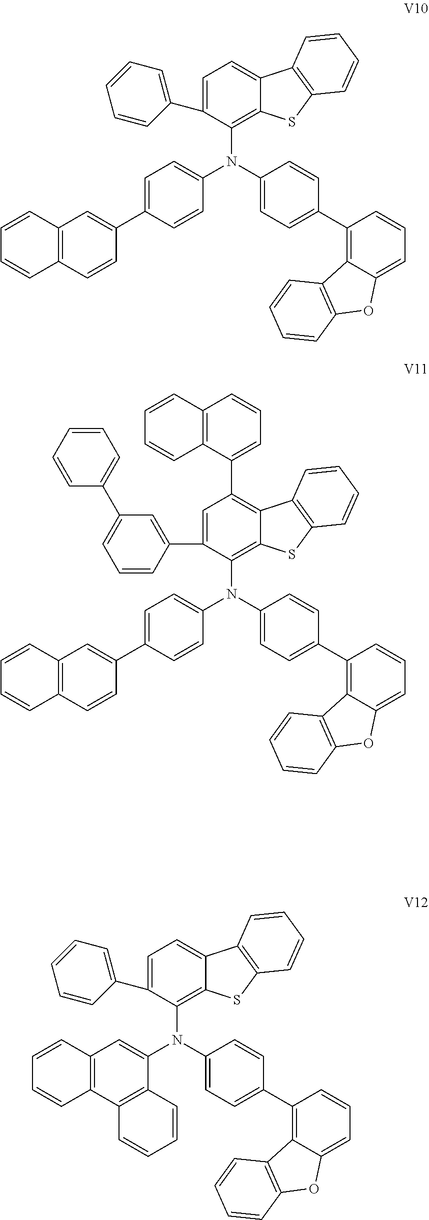 [SMILES: c1ccc(-c2ccc3c(sc4ccccc43)c2N(c2ccc(-c3ccc4ccccc4c3)cc2)c2ccc(-c3cccc4oc5ccccc5c34)cc2)cc1 ; c1ccc(-c2cccc(-c3cc(-c4cccc5ccccc45)c4c(sc5ccccc54)c3N(c3ccc(-c4ccc5ccccc5c4)cc3)c3ccc(-c4cccc5oc6ccccc6c45)cc3)c2)cc1 ; c1ccc(-c2ccc3c(sc4ccccc43)c2N(c2ccc(-c3cccc4oc5ccccc5c34)cc2)c2cc3ccccc3c3ccccc23)cc1]

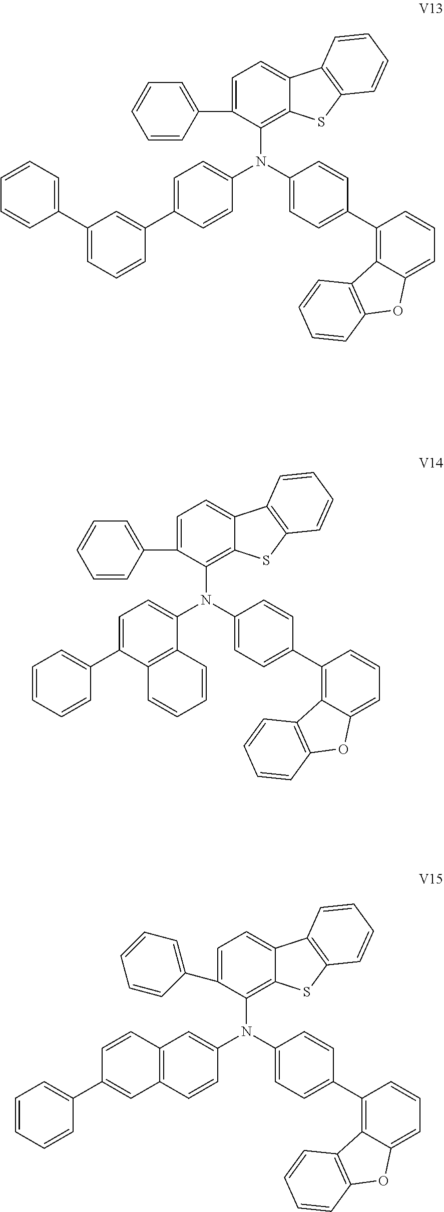 [SMILES: c1ccc(-c2cccc(-c3ccc(N(c4ccc(-c5cccc6oc7ccccc7c56)cc4)c4c(-c5ccccc5)ccc5c4sc4ccccc45)cc3)c2)cc1 ; c1ccc(-c2ccc3c(sc4ccccc43)c2N(c2ccc(-c3cccc4oc5ccccc5c34)cc2)c2ccc(-c3ccccc3)c3ccccc23)cc1 ; c1ccc(-c2ccc3cc(N(c4ccc(-c5cccc6oc7ccccc7c56)cc4)c4c(-c5ccccc5)ccc5c4sc4ccccc45)ccc3c2)cc1]

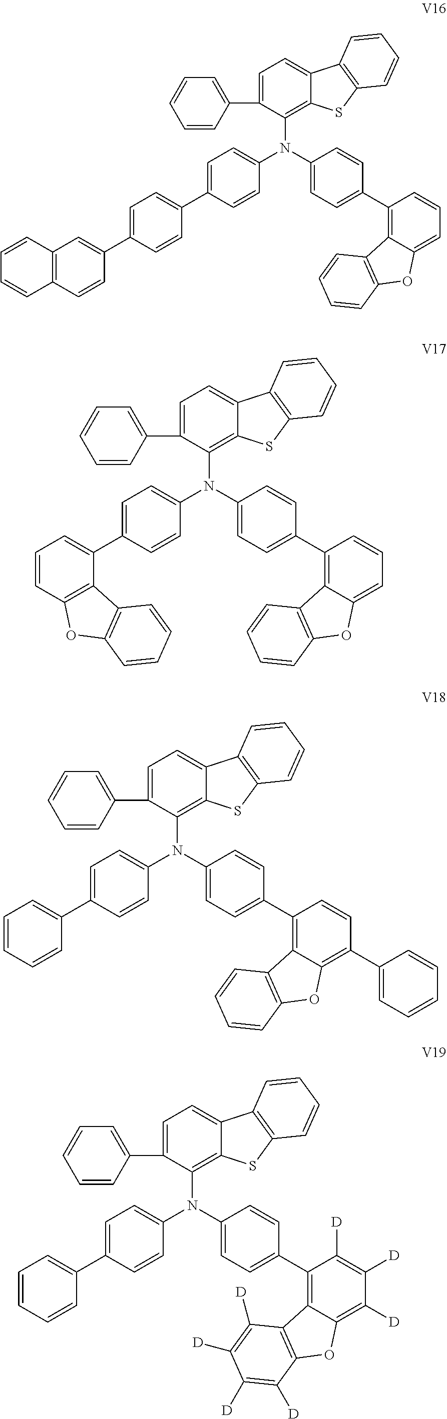 [SMILES: c1ccc(-c2ccc3c(sc4ccccc43)c2N(c2ccc(-c3ccc(-c4ccc5ccccc5c4)cc3)cc2)c2ccc(-c3cccc4oc5ccccc5c34)cc2)cc1 ; c1ccc(-c2ccc3c(sc4ccccc43)c2N(c2ccc(-c3cccc4oc5ccccc5c34)cc2)c2ccc(-c3cccc4oc5ccccc5c34)cc2)cc1 ; c1ccc(-c2ccc(N(c3ccc(-c4ccc(-c5ccccc5)c5oc6ccccc6c45)cc3)c3c(-c4ccccc4)ccc4c3sc3ccccc34)cc2)cc1 ; [2H]c1c([2H])c([2H])c2c(oc3c([2H])c([2H])c([2H])c(-c4ccc(N(c5ccc(-c6ccccc6)cc5)c5c(-c6ccccc6)ccc6c5sc5ccccc56)cc4)c32)c1[2H]]

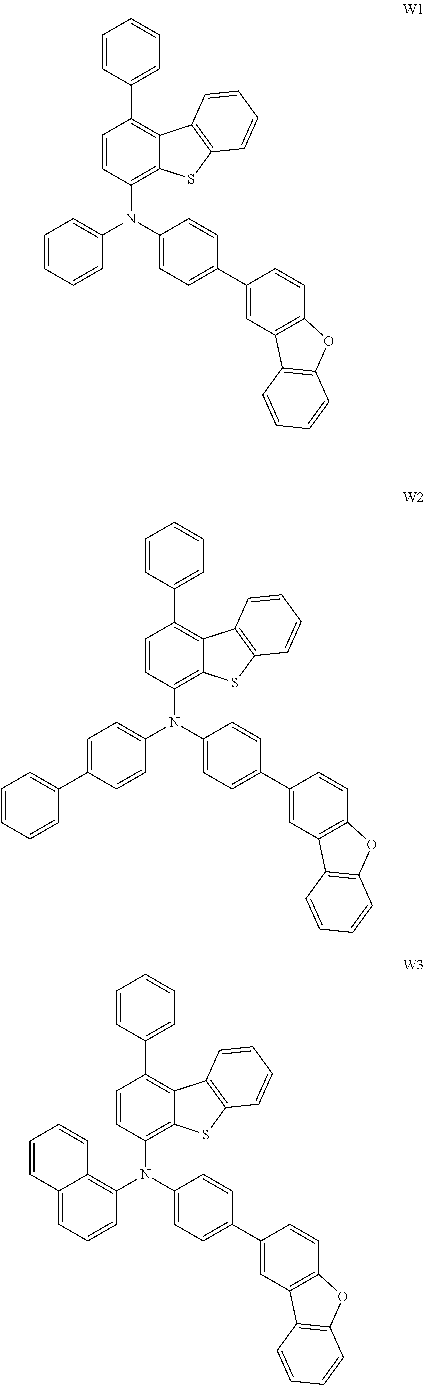 [SMILES: c1ccc(-c2ccc(N(c3ccccc3)c3ccc(-c4ccc5oc6ccccc6c5c4)cc3)c3sc4ccccc4c23)cc1 ; c1ccc(-c2ccc(N(c3ccc(-c4ccc5oc6ccccc6c5c4)cc3)c3ccc(-c4ccccc4)c4c3sc3ccccc34)cc2)cc1 ; c1ccc(-c2ccc(N(c3ccc(-c4ccc5oc6ccccc6c5c4)cc3)c3cccc4ccccc34)c3sc4ccccc4c23)cc1]

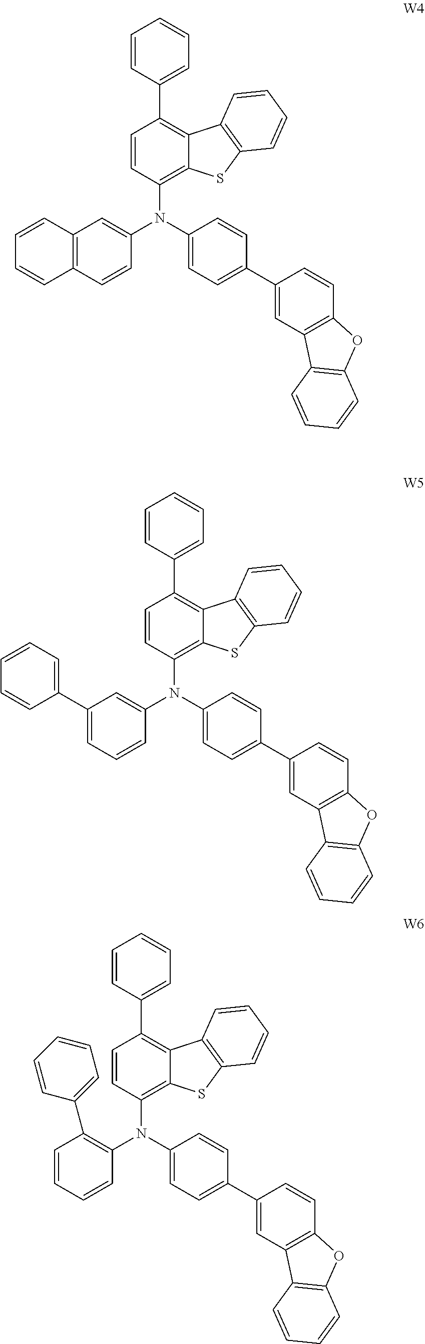 [SMILES: c1ccc(-c2ccc(N(c3ccc(-c4ccc5oc6ccccc6c5c4)cc3)c3ccc4ccccc4c3)c3sc4ccccc4c23)cc1 ; c1ccc(-c2cccc(N(c3ccc(-c4ccc5oc6ccccc6c5c4)cc3)c3ccc(-c4ccccc4)c4c3sc3ccccc34)c2)cc1 ; c1ccc(-c2ccccc2N(c2ccc(-c3ccc4oc5ccccc5c4c3)cc2)c2ccc(-c3ccccc3)c3c2sc2ccccc23)cc1]

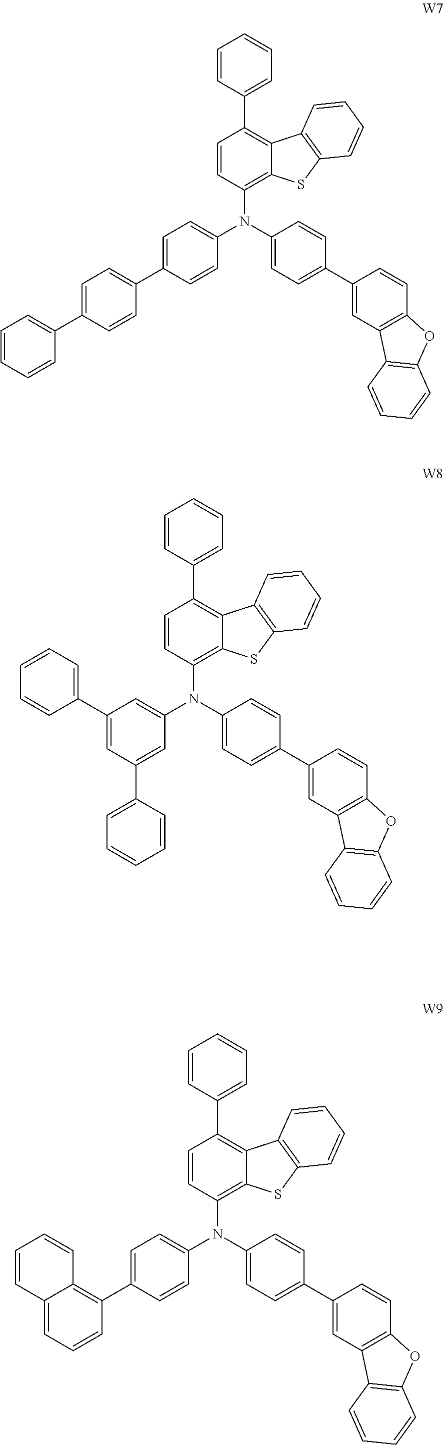 [SMILES: c1ccc(-c2ccc(-c3ccc(N(c4ccc(-c5ccc6oc7ccccc7c6c5)cc4)c4ccc(-c5ccccc5)c5c4sc4ccccc45)cc3)cc2)cc1 ; c1ccc(-c2cc(-c3ccccc3)cc(N(c3ccc(-c4ccc5oc6ccccc6c5c4)cc3)c3ccc(-c4ccccc4)c4c3sc3ccccc34)c2)cc1 ; c1ccc(-c2ccc(N(c3ccc(-c4ccc5oc6ccccc6c5c4)cc3)c3ccc(-c4cccc5ccccc45)cc3)c3sc4ccccc4c23)cc1]

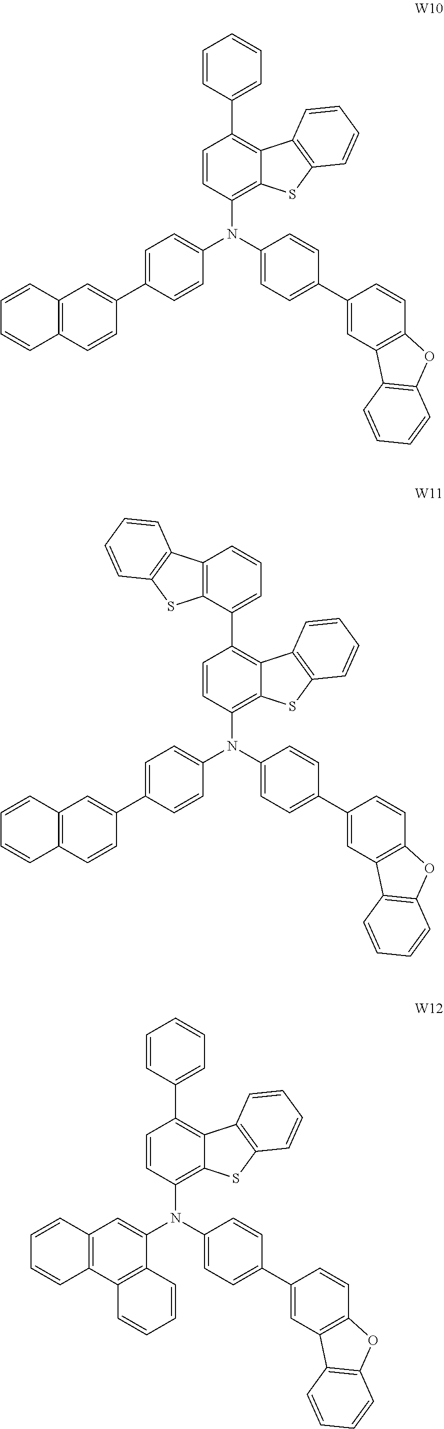 [SMILES: c1ccc(-c2ccc(N(c3ccc(-c4ccc5ccccc5c4)cc3)c3ccc(-c4ccc5oc6ccccc6c5c4)cc3)c3sc4ccccc4c23)cc1 ; c1ccc2cc(-c3ccc(N(c4ccc(-c5ccc6oc7ccccc7c6c5)cc4)c4ccc(-c5cccc6c5sc5ccccc56)c5c4sc4ccccc45)cc3)ccc2c1 ; c1ccc(-c2ccc(N(c3ccc(-c4ccc5oc6ccccc6c5c4)cc3)c3cc4ccccc4c4ccccc34)c3sc4ccccc4c23)cc1]

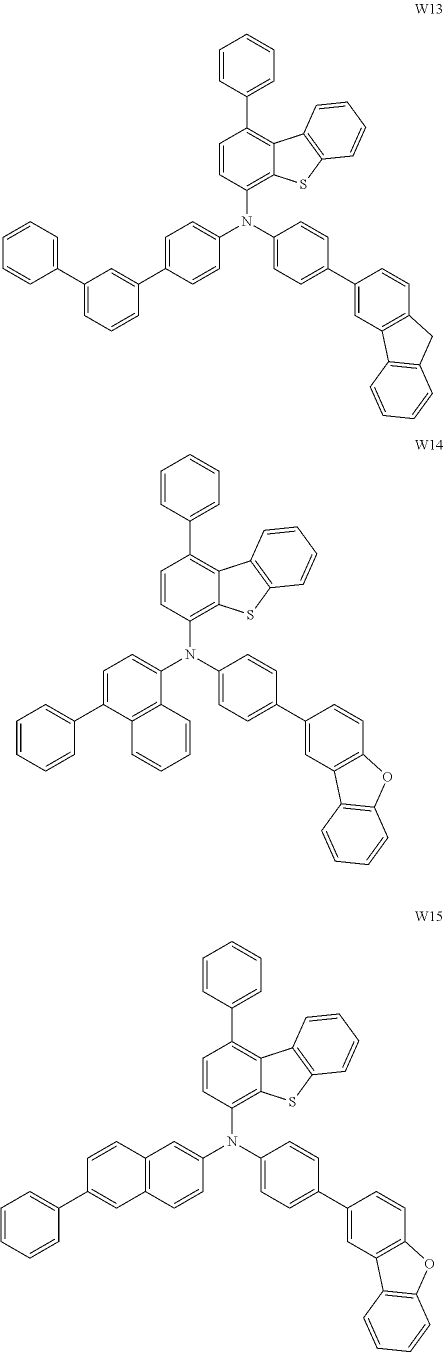 [SMILES: c1ccc(-c2cccc(-c3ccc(N(c4ccc(-c5ccc6c(c5)-c5ccccc5C6)cc4)c4ccc(-c5ccccc5)c5c4sc4ccccc45)cc3)c2)cc1 ; c1ccc(-c2ccc(N(c3ccc(-c4ccc5oc6ccccc6c5c4)cc3)c3ccc(-c4ccccc4)c4c3sc3ccccc34)c3ccccc23)cc1 ; c1ccc(-c2ccc3cc(N(c4ccc(-c5ccc6oc7ccccc7c6c5)cc4)c4ccc(-c5ccccc5)c5c4sc4ccccc45)ccc3c2)cc1]

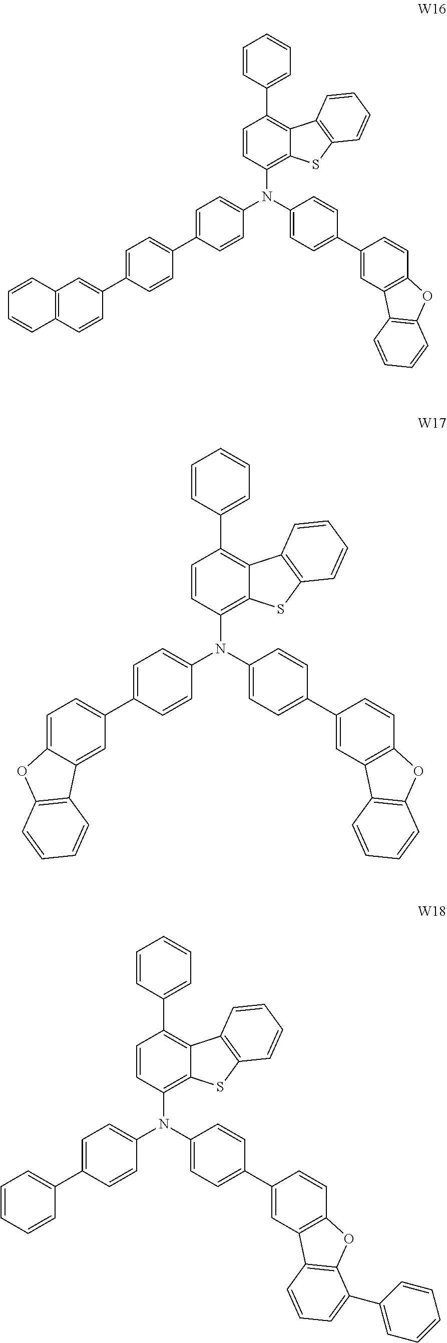 [SMILES: c1ccc(-c2ccc(N(c3ccc(-c4ccc(-c5ccc6ccccc6c5)cc4)cc3)c3ccc(-c4ccc5oc6ccccc6c5c4)cc3)c3sc4ccccc4c23)cc1 ; c1ccc(-c2ccc(N(c3ccc(-c4ccc5oc6ccccc6c5c4)cc3)c3ccc(-c4ccc5oc6ccccc6c5c4)cc3)c3sc4ccccc4c23)cc1 ; c1ccc(-c2ccc(N(c3ccc(-c4ccc5oc6c(-c7ccccc7)cccc6c5c4)cc3)c3ccc(-c4ccccc4)c4c3sc3ccccc34)cc2)cc1]

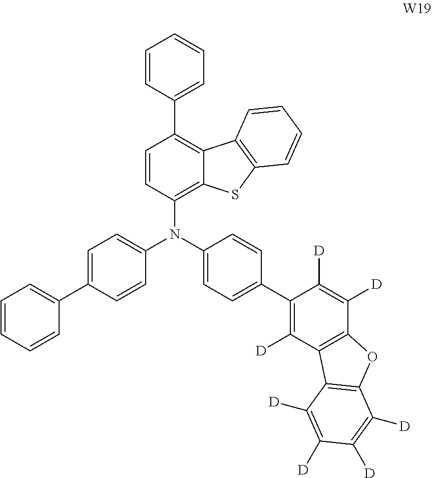 [SMILES: [2H]c1c([2H])c([2H])c2c(oc3c([2H])c([2H])c(-c4ccc(N(c5ccc(-c6ccccc6)cc5)c5ccc(-c6ccccc6)c6c5sc5ccccc56)cc4)c([2H])c32)c1[2H]]

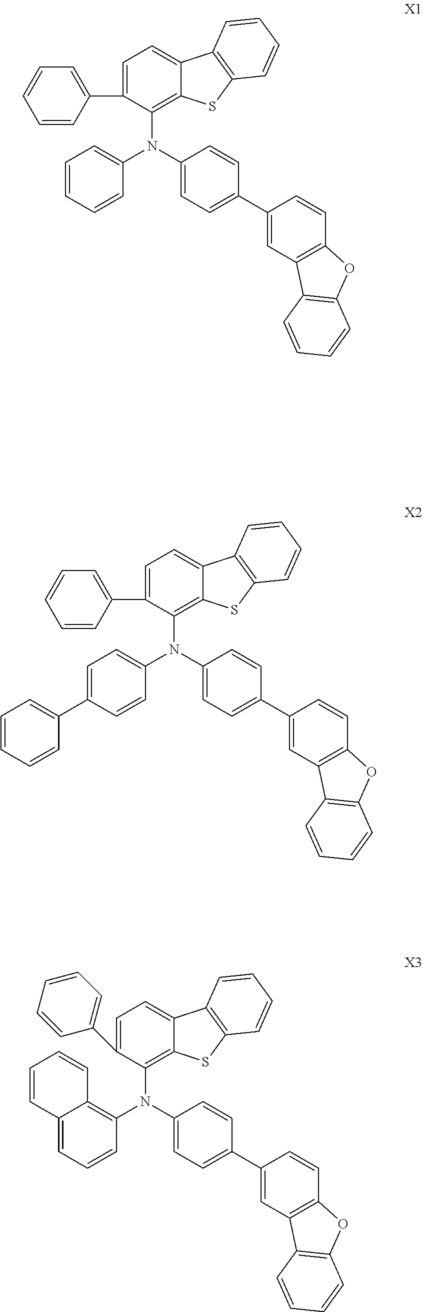 [SMILES: c1ccc(-c2ccc3c(sc4ccccc43)c2N(c2ccccc2)c2ccc(-c3ccc4oc5ccccc5c4c3)cc2)cc1 ; c1ccc(-c2ccc(N(c3ccc(-c4ccc5oc6ccccc6c5c4)cc3)c3c(-c4ccccc4)ccc4c3sc3ccccc34)cc2)cc1 ; c1ccc(-c2ccc3c(sc4ccccc43)c2N(c2ccc(-c3ccc4oc5ccccc5c4c3)cc2)c2cccc3ccccc23)cc1]

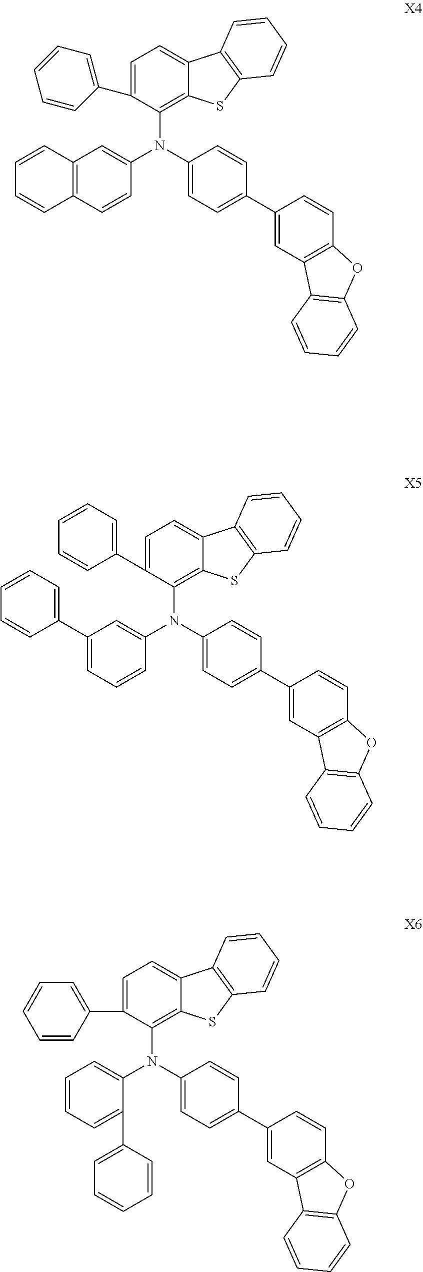 [SMILES: c1ccc(-c2ccc3c(sc4ccccc43)c2N(c2ccc(-c3ccc4oc5ccccc5c4c3)cc2)c2ccc3ccccc3c2)cc1 ; c1ccc(-c2cccc(N(c3ccc(-c4ccc5oc6ccccc6c5c4)cc3)c3c(-c4ccccc4)ccc4c3sc3ccccc34)c2)cc1 ; c1ccc(-c2ccccc2N(c2ccc(-c3ccc4oc5ccccc5c4c3)cc2)c2c(-c3ccccc3)ccc3c2sc2ccccc23)cc1]

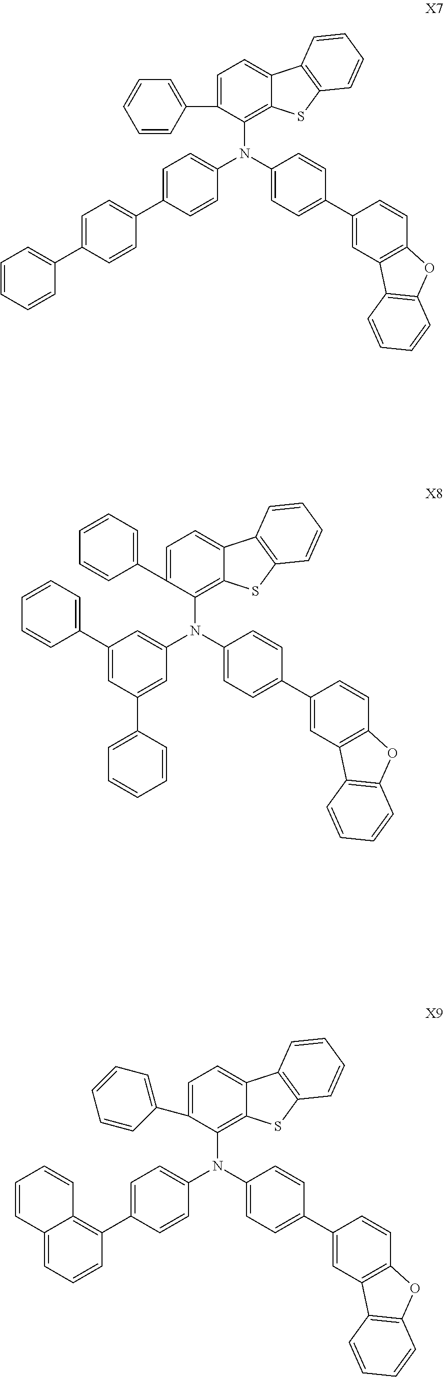 [SMILES: c1ccc(-c2ccc(-c3ccc(N(c4ccc(-c5ccc6oc7ccccc7c6c5)cc4)c4c(-c5ccccc5)ccc5c4sc4ccccc45)cc3)cc2)cc1 ; c1ccc(-c2cc(-c3ccccc3)cc(N(c3ccc(-c4ccc5oc6ccccc6c5c4)cc3)c3c(-c4ccccc4)ccc4c3sc3ccccc34)c2)cc1 ; c1ccc(-c2ccc3c(sc4ccccc43)c2N(c2ccc(-c3ccc4oc5ccccc5c4c3)cc2)c2ccc(-c3cccc4ccccc34)cc2)cc1]

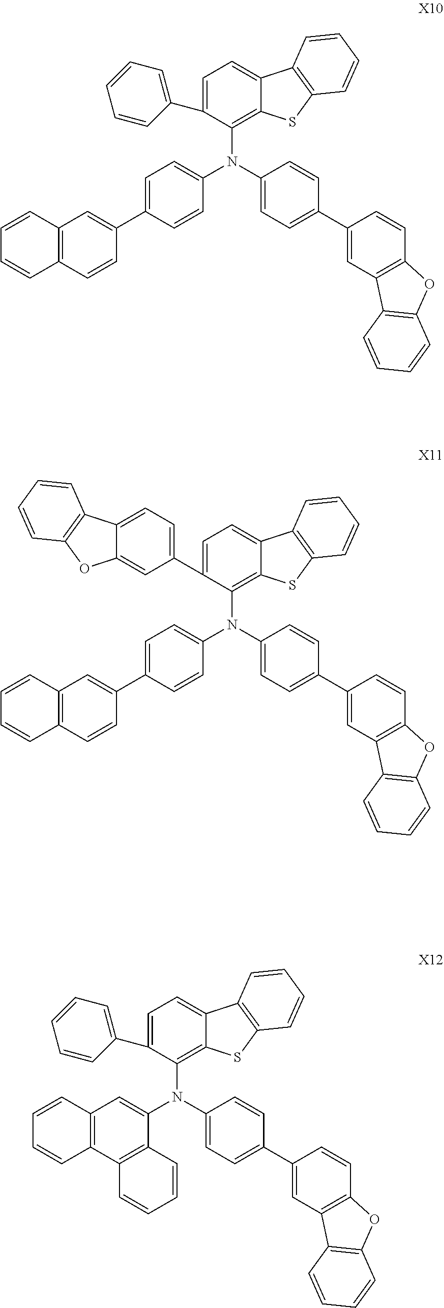 [SMILES: c1ccc(-c2ccc3c(sc4ccccc43)c2N(c2ccc(-c3ccc4ccccc4c3)cc2)c2ccc(-c3ccc4oc5ccccc5c4c3)cc2)cc1 ; c1ccc2cc(-c3ccc(N(c4ccc(-c5ccc6oc7ccccc7c6c5)cc4)c4c(-c5ccc6c(c5)oc5ccccc56)ccc5c4sc4ccccc45)cc3)ccc2c1 ; c1ccc(-c2ccc3c(sc4ccccc43)c2N(c2ccc(-c3ccc4oc5ccccc5c4c3)cc2)c2cc3ccccc3c3ccccc23)cc1]

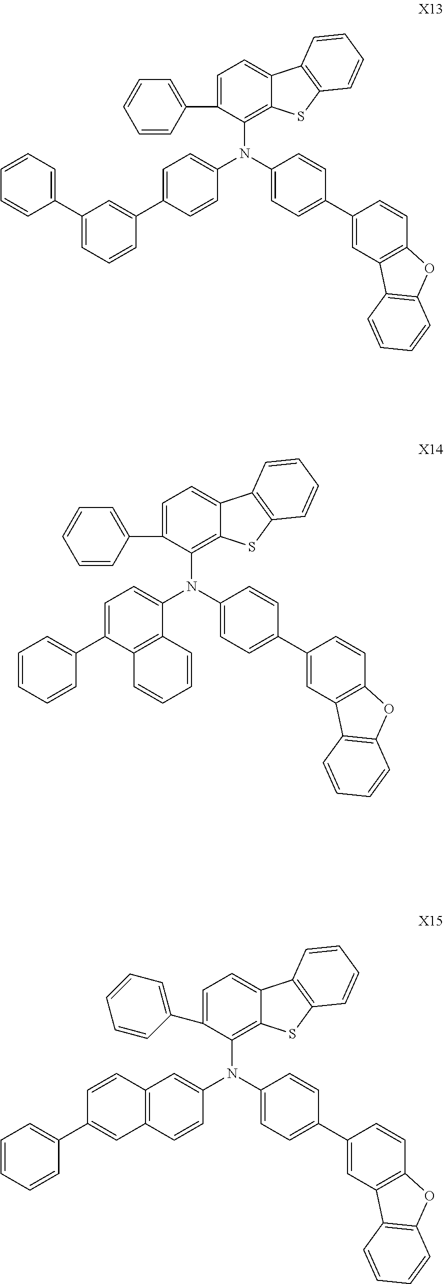 [SMILES: c1ccc(-c2cccc(-c3ccc(N(c4ccc(-c5ccc6oc7ccccc7c6c5)cc4)c4c(-c5ccccc5)ccc5c4sc4ccccc45)cc3)c2)cc1 ; c1ccc(-c2ccc3c(sc4ccccc43)c2N(c2ccc(-c3ccc4oc5ccccc5c4c3)cc2)c2ccc(-c3ccccc3)c3ccccc23)cc1 ; c1ccc(-c2ccc3cc(N(c4ccc(-c5ccc6oc7ccccc7c6c5)cc4)c4c(-c5ccccc5)ccc5c4sc4ccccc45)ccc3c2)cc1]

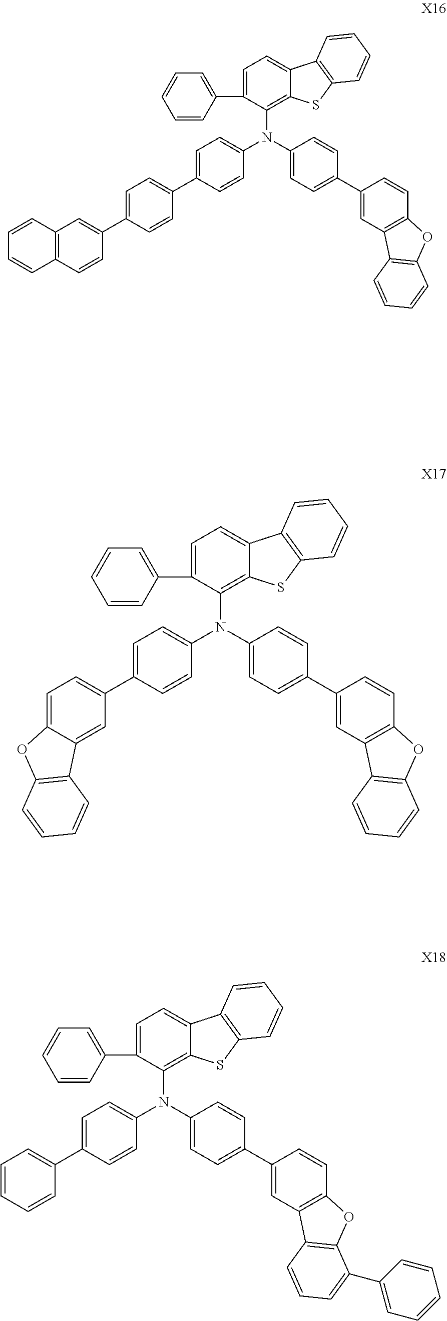 [SMILES: c1ccc(-c2ccc3c(sc4ccccc43)c2N(c2ccc(-c3ccc(-c4ccc5ccccc5c4)cc3)cc2)c2ccc(-c3ccc4oc5ccccc5c4c3)cc2)cc1 ; c1ccc(-c2ccc3c(sc4ccccc43)c2N(c2ccc(-c3ccc4oc5ccccc5c4c3)cc2)c2ccc(-c3ccc4oc5ccccc5c4c3)cc2)cc1 ; c1ccc(-c2ccc(N(c3ccc(-c4ccc5oc6c(-c7ccccc7)cccc6c5c4)cc3)c3c(-c4ccccc4)ccc4c3sc3ccccc34)cc2)cc1]

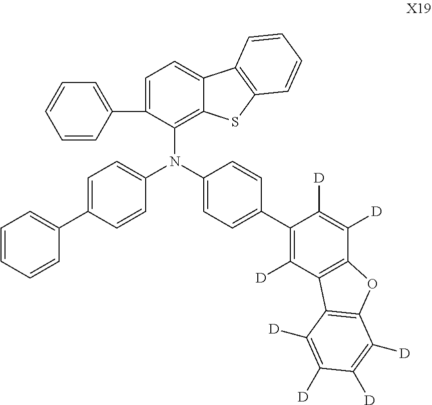 [SMILES: [2H]c1c([2H])c([2H])c2c(oc3c([2H])c([2H])c(-c4ccc(N(c5ccc(-c6ccccc6)cc5)c5c(-c6ccccc6)ccc6c5sc5ccccc56)cc4)c([2H])c32)c1[2H]]

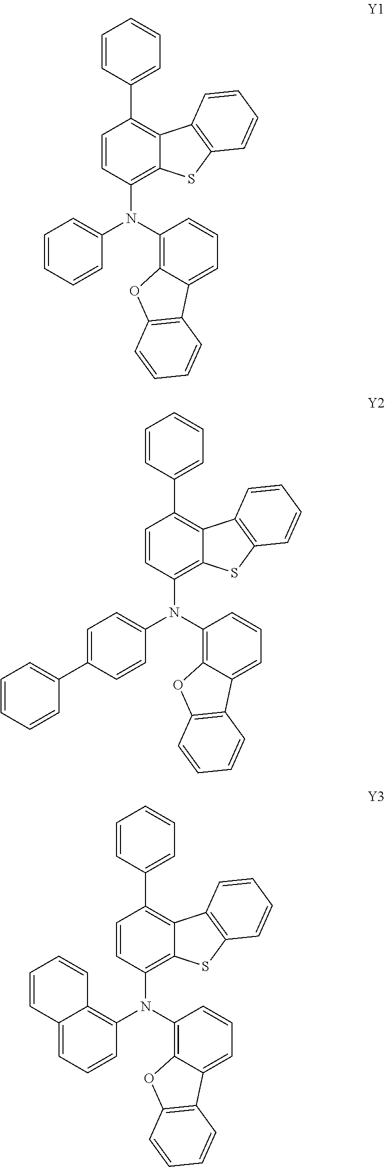

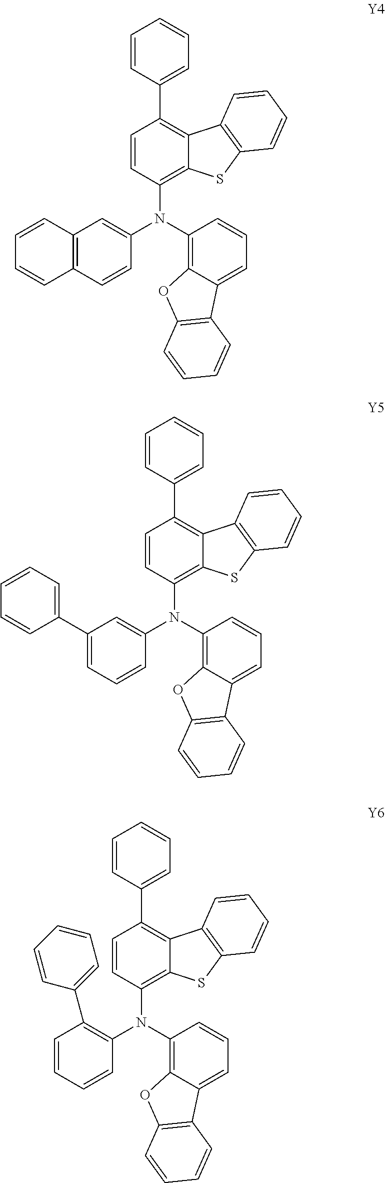

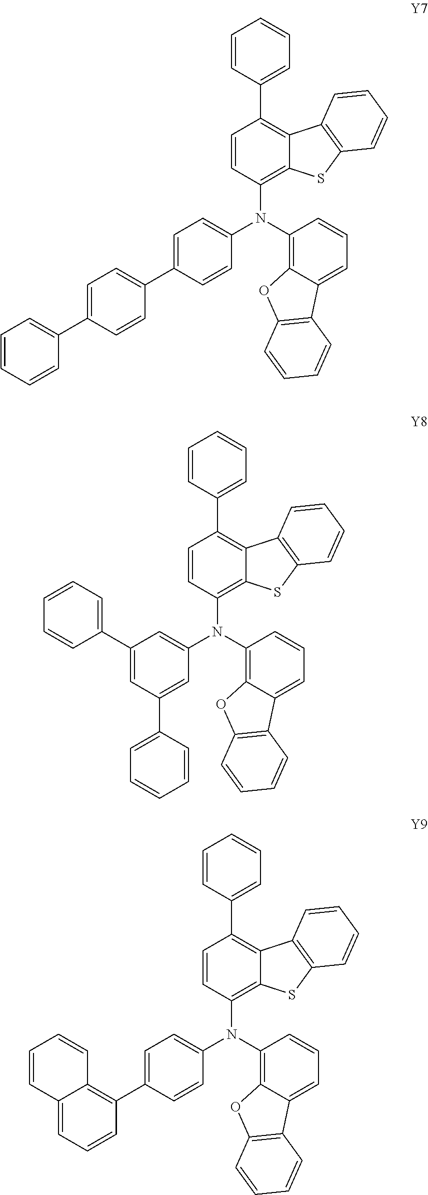

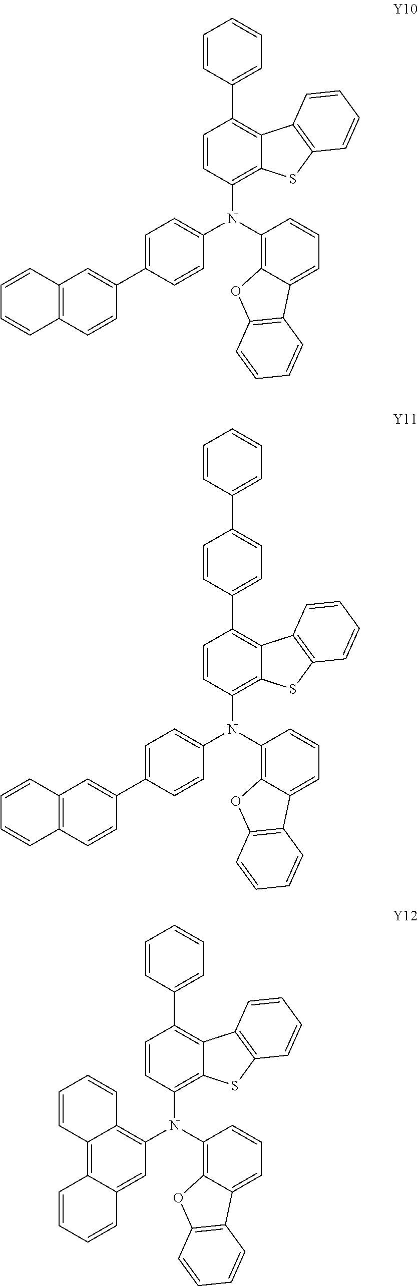 [SMILES: c1ccc(-c2ccc(N(c3ccc(-c4ccc5ccccc5c4)cc3)c3cccc4c3oc3ccccc34)c3sc4ccccc4c23)cc1 ; c1ccc(-c2ccc(-c3ccc(N(c4ccc(-c5ccc6ccccc6c5)cc4)c4cccc5c4oc4ccccc45)c4sc5ccccc5c34)cc2)cc1 ; c1ccc(-c2ccc(N(c3cc4ccccc4c4ccccc34)c3cccc4c3oc3ccccc34)c3sc4ccccc4c23)cc1]

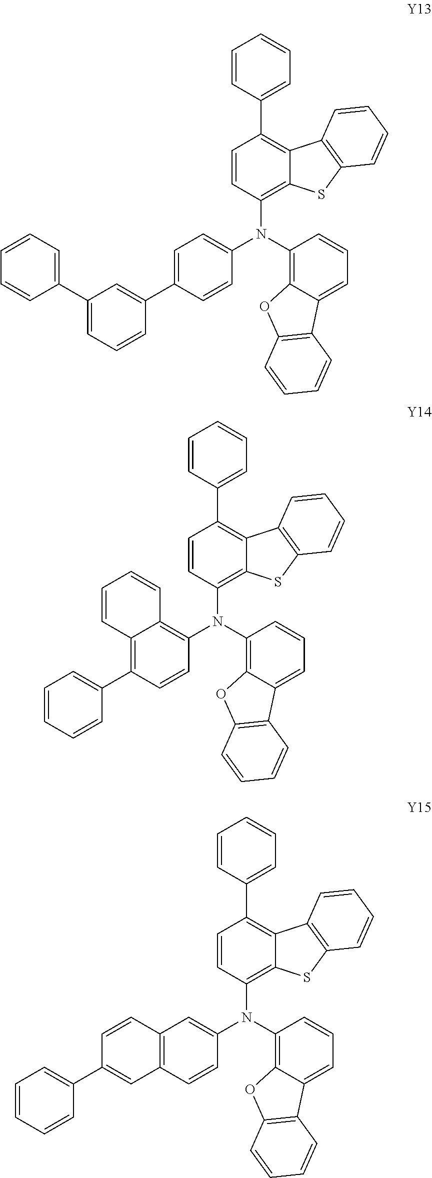

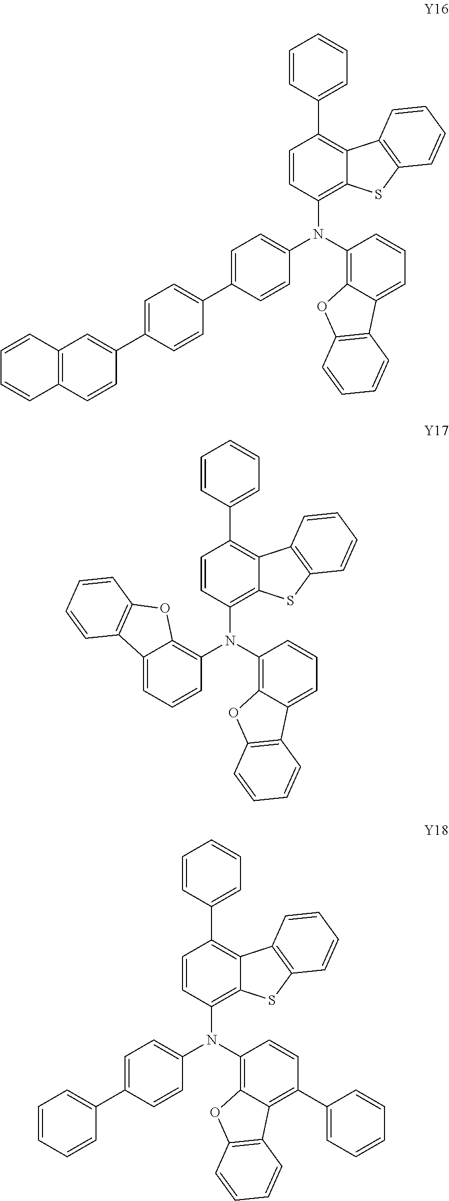 [SMILES: c1ccc(-c2ccc(N(c3ccc(-c4ccc(-c5ccc6ccccc6c5)cc4)cc3)c3cccc4c3oc3ccccc34)c3sc4ccccc4c23)cc1 ; c1ccc(-c2ccc(N(c3cccc4c3oc3ccccc34)c3cccc4c3oc3ccccc34)c3sc4ccccc4c23)cc1 ; c1ccc(-c2ccc(N(c3ccc(-c4ccccc4)c4c3oc3ccccc34)c3ccc(-c4ccccc4)c4c3sc3ccccc34)cc2)cc1]

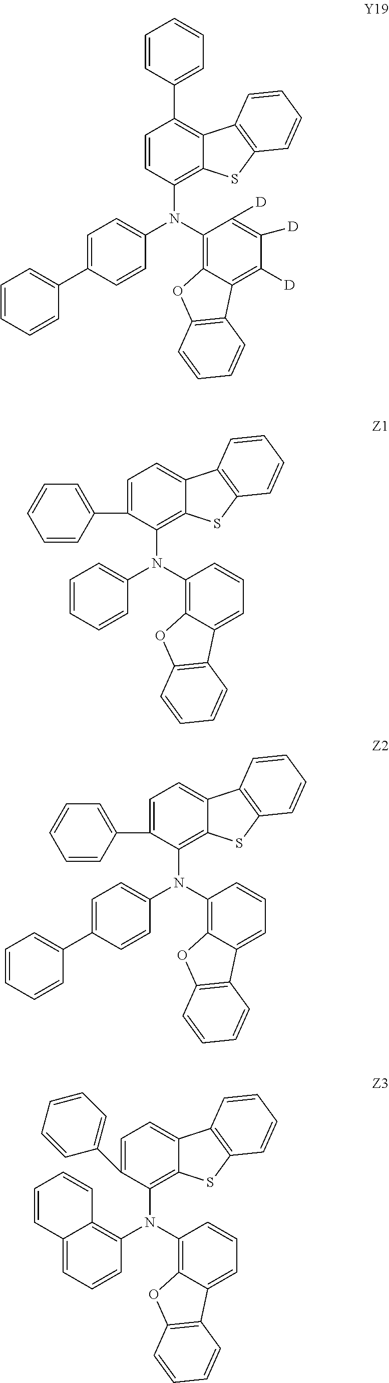 [SMILES: [2H]c1c([2H])c([2H])c2c(oc3ccccc32)c1N(c1ccc(-c2ccccc2)cc1)c1ccc(-c2ccccc2)c2c1sc1ccccc12 ; c1ccc(-c2ccc3c(sc4ccccc43)c2N(c2ccccc2)c2cccc3c2oc2ccccc23)cc1 ; c1ccc(-c2ccc(N(c3cccc4c3oc3ccccc34)c3c(-c4ccccc4)ccc4c3sc3ccccc34)cc2)cc1 ; c1ccc(-c2ccc3c(sc4ccccc43)c2N(c2cccc3ccccc23)c2cccc3c2oc2ccccc23)cc1]

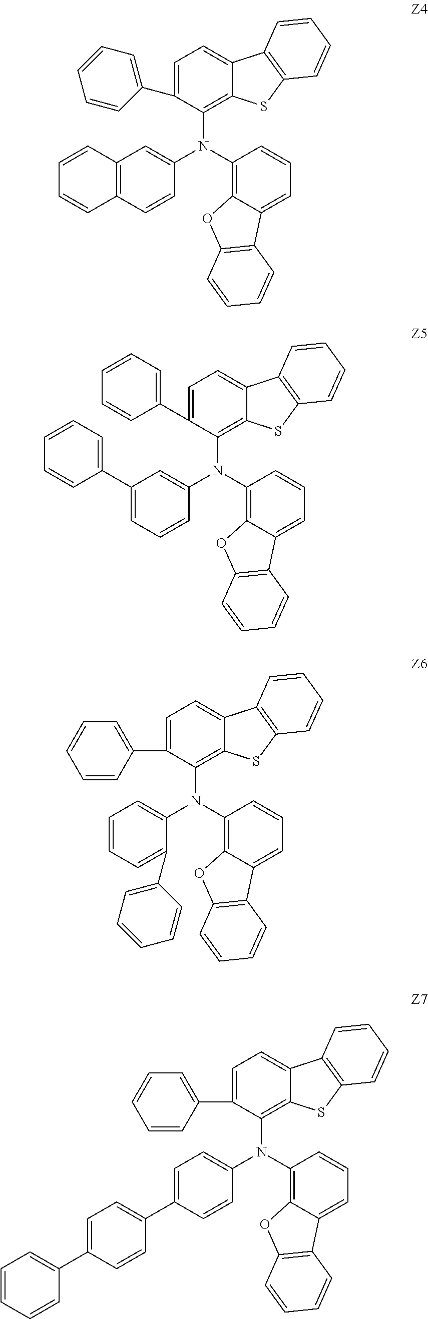 [SMILES: c1ccc(-c2ccc3c(sc4ccccc43)c2N(c2ccc3ccccc3c2)c2cccc3c2oc2ccccc23)cc1 ; c1ccc(-c2cccc(N(c3cccc4c3oc3ccccc34)c3c(-c4ccccc4)ccc4c3sc3ccccc34)c2)cc1 ; c1ccc(-c2ccccc2N(c2cccc3c2oc2ccccc23)c2c(-c3ccccc3)ccc3c2sc2ccccc23)cc1 ; c1ccc(-c2ccc(-c3ccc(N(c4cccc5c4oc4ccccc45)c4c(-c5ccccc5)ccc5c4sc4ccccc45)cc3)cc2)cc1]

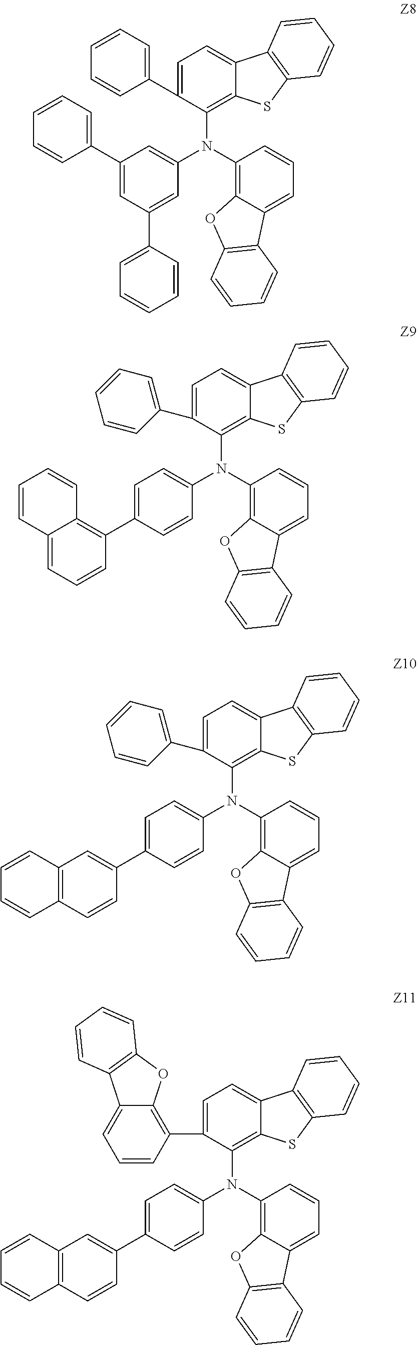 [SMILES: c1ccc(-c2cc(-c3ccccc3)cc(N(c3cccc4c3oc3ccccc34)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2cccc3c2oc2ccccc23)cc1 ; c1ccc(-c2ccc3c(sc4ccccc43)c2N(c2ccc(-c3ccc4ccccc4c3)cc2)c2cccc3c2oc2ccccc23)cc1 ; c1ccc2cc(-c3ccc(N(c4cccc5c4oc4ccccc45)c4c(-c5cccc6c5oc5ccccc56)ccc5c4sc4ccccc45)cc3)ccc2c1]

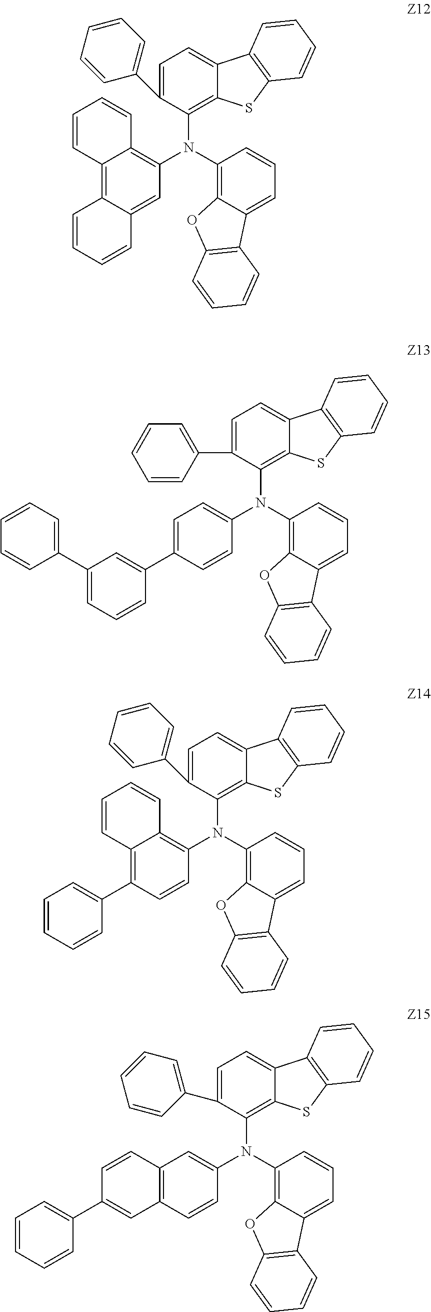

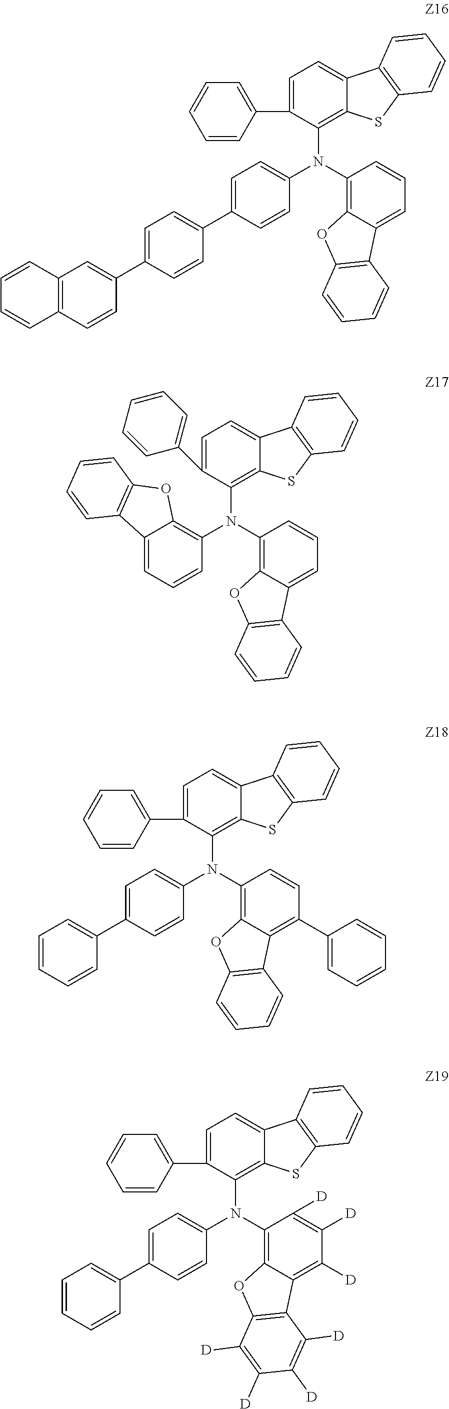 [SMILES: c1ccc(-c2ccc3c(sc4ccccc43)c2N(c2ccc(-c3ccc(-c4ccc5ccccc5c4)cc3)cc2)c2cccc3c2oc2ccccc23)cc1 ; c1ccc(-c2ccc3c(sc4ccccc43)c2N(c2cccc3c2oc2ccccc23)c2cccc3c2oc2ccccc23)cc1 ; c1ccc(-c2ccc(N(c3ccc(-c4ccccc4)c4c3oc3ccccc34)c3c(-c4ccccc4)ccc4c3sc3ccccc34)cc2)cc1 ; [2H]c1c([2H])c([2H])c2c(oc3c(N(c4ccc(-c5ccccc5)cc4)c4c(-c5ccccc5)ccc5c4sc4ccccc45)c([2H])c([2H])c([2H])c32)c1[2H]]

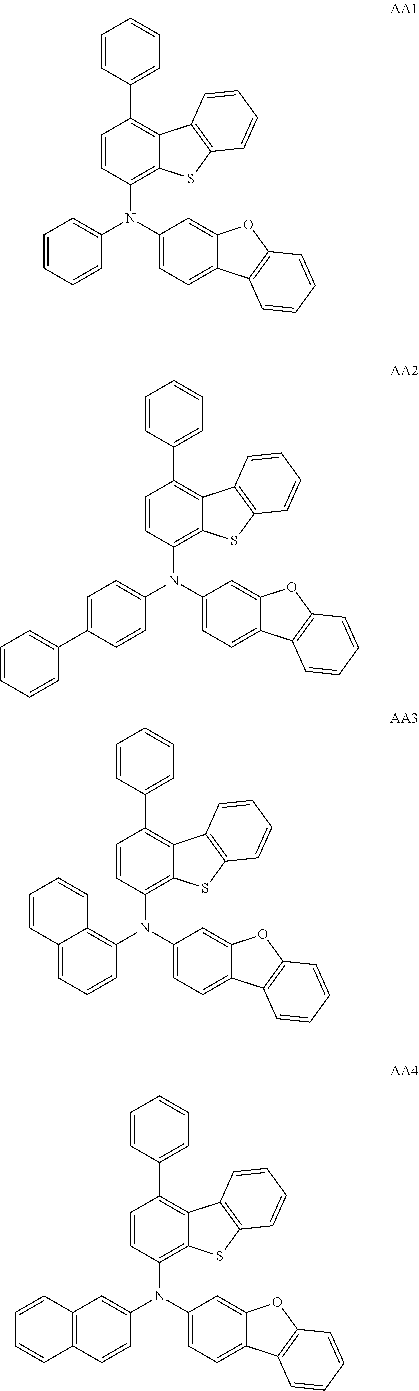 [SMILES: c1ccc(-c2ccc(N(c3ccccc3)c3ccc4c(c3)oc3ccccc34)c3sc4ccccc4c23)cc1 ; c1ccc(-c2ccc(N(c3ccc4c(c3)oc3ccccc34)c3ccc(-c4ccccc4)c4c3sc3ccccc34)cc2)cc1 ; c1ccc(-c2ccc(N(c3ccc4c(c3)oc3ccccc34)c3cccc4ccccc34)c3sc4ccccc4c23)cc1 ; c1ccc(-c2ccc(N(c3ccc4ccccc4c3)c3ccc4c(c3)oc3ccccc34)c3sc4ccccc4c23)cc1]

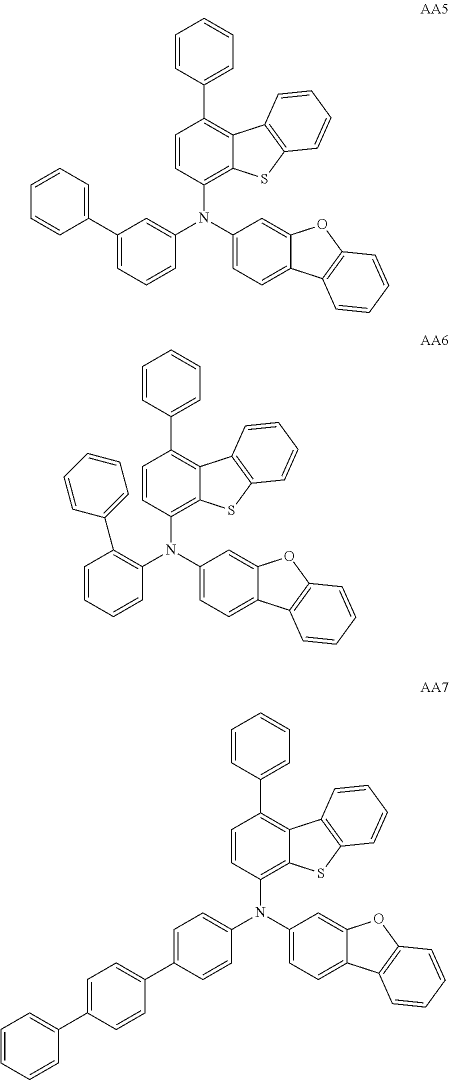 [SMILES: c1ccc(-c2cccc(N(c3ccc4c(c3)oc3ccccc34)c3ccc(-c4ccccc4)c4c3sc3ccccc34)c2)cc1 ; c1ccc(-c2ccccc2N(c2ccc3c(c2)oc2ccccc23)c2ccc(-c3ccccc3)c3c2sc2ccccc23)cc1 ; c1ccc(-c2ccc(-c3ccc(N(c4ccc5c(c4)oc4ccccc45)c4ccc(-c5ccccc5)c5c4sc4ccccc45)cc3)cc2)cc1]

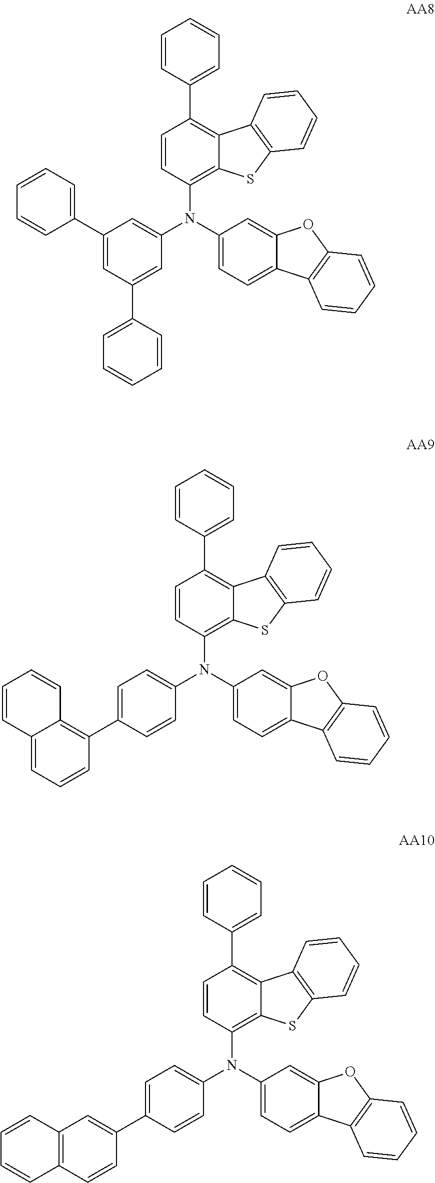 [SMILES: c1ccc(-c2cc(-c3ccccc3)cc(N(c3ccc4c(c3)oc3ccccc34)c3ccc(-c4ccccc4)c4c3sc3ccccc34)c2)cc1 ; c1ccc(-c2ccc(N(c3ccc(-c4cccc5ccccc45)cc3)c3ccc4c(c3)oc3ccccc34)c3sc4ccccc4c23)cc1 ; c1ccc(-c2ccc(N(c3ccc(-c4ccc5ccccc5c4)cc3)c3ccc4c(c3)oc3ccccc34)c3sc4ccccc4c23)cc1]

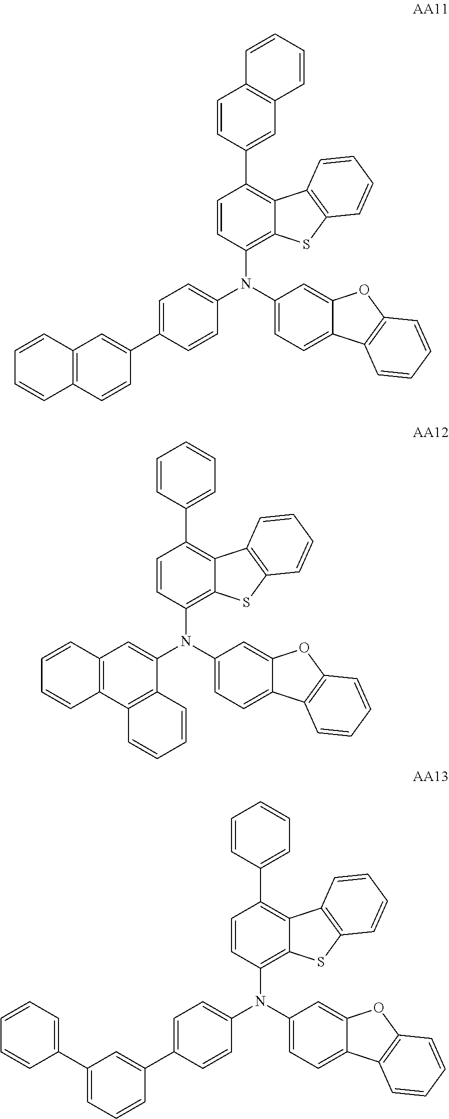 [SMILES: c1ccc2cc(-c3ccc(N(c4ccc5c(c4)oc4ccccc45)c4ccc(-c5ccc6ccccc6c5)c5c4sc4ccccc45)cc3)ccc2c1 ; c1ccc(-c2ccc(N(c3ccc4c(c3)oc3ccccc34)c3cc4ccccc4c4ccccc34)c3sc4ccccc4c23)cc1 ; c1ccc(-c2cccc(-c3ccc(N(c4ccc5c(c4)oc4ccccc45)c4ccc(-c5ccccc5)c5c4sc4ccccc45)cc3)c2)cc1]

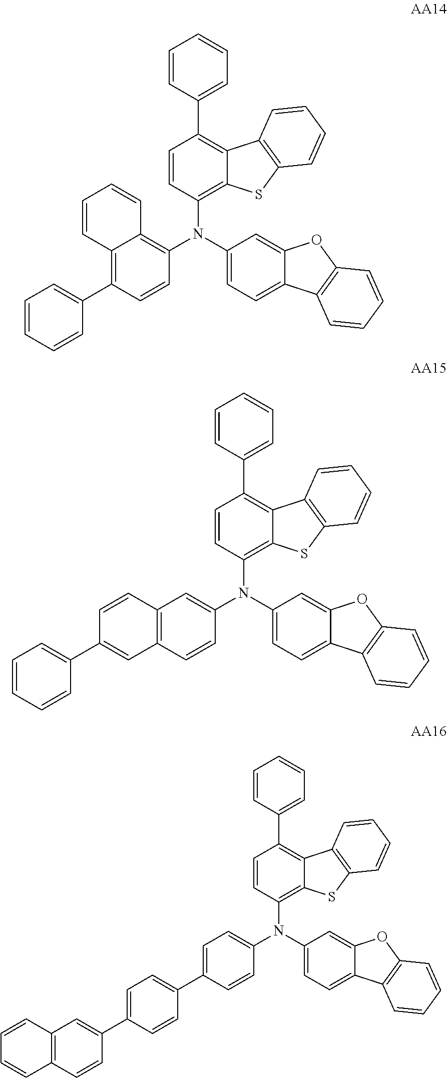 [SMILES: c1ccc(-c2ccc(N(c3ccc4c(c3)oc3ccccc34)c3ccc(-c4ccccc4)c4c3sc3ccccc34)c3ccccc23)cc1 ; c1ccc(-c2ccc3cc(N(c4ccc5c(c4)oc4ccccc45)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc4c(c3)oc3ccccc34)c3sc4ccccc4c23)cc1]

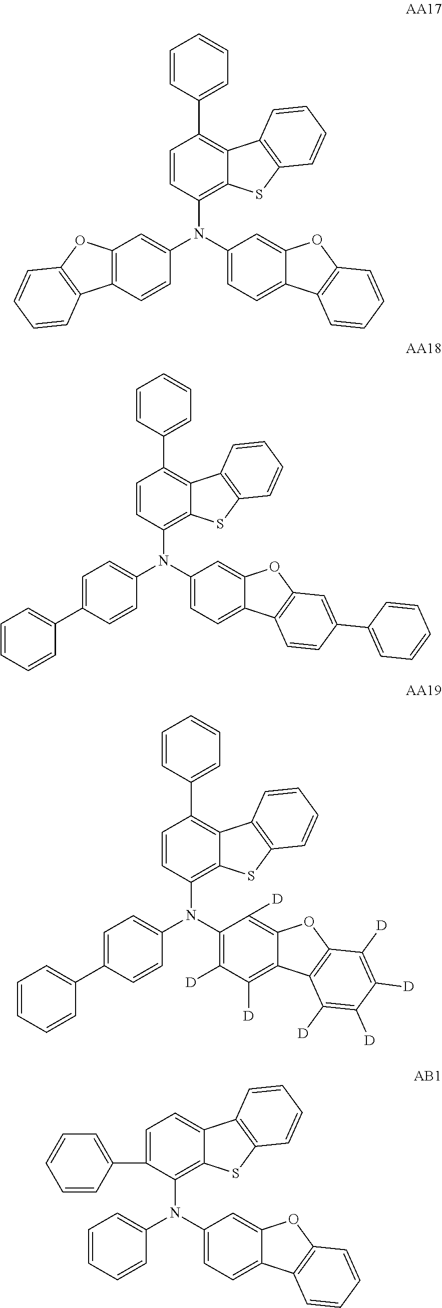 [SMILES: c1ccc(-c2ccc(N(c3ccc4c(c3)oc3ccccc34)c3ccc4c(c3)oc3ccccc34)c3sc4ccccc4c23)cc1 ; c1ccc(-c2ccc(N(c3ccc4c(c3)oc3cc(-c5ccccc5)ccc34)c3ccc(-c4ccccc4)c4c3sc3ccccc34)cc2)cc1 ; [2H]c1c([2H])c([2H])c2c(oc3c([2H])c(N(c4ccc(-c5ccccc5)cc4)c4ccc(-c5ccccc5)c5c4sc4ccccc45)c([2H])c([2H])c32)c1[2H] ; c1ccc(-c2ccc3c(sc4ccccc43)c2N(c2ccccc2)c2ccc3c(c2)oc2ccccc23)cc1]

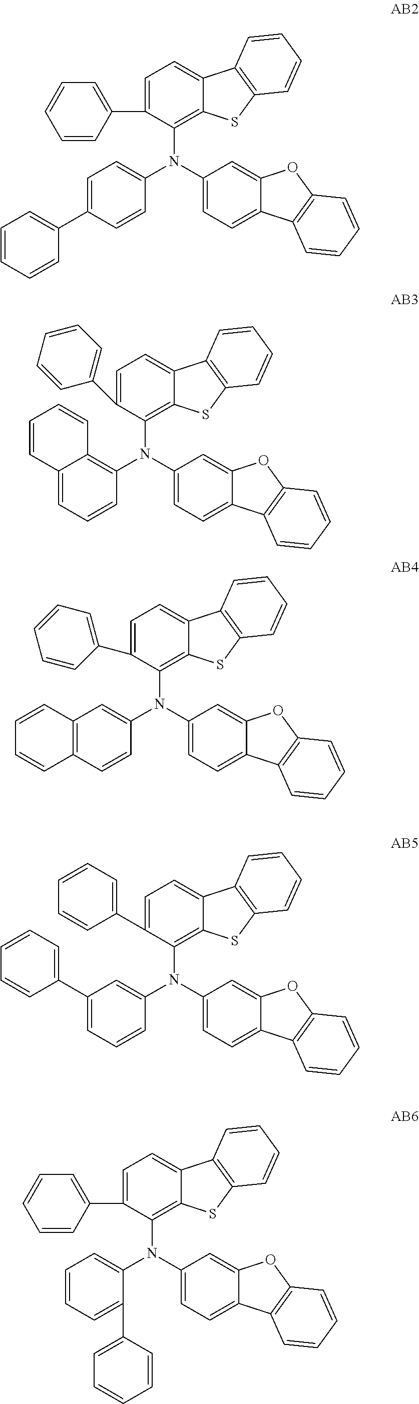

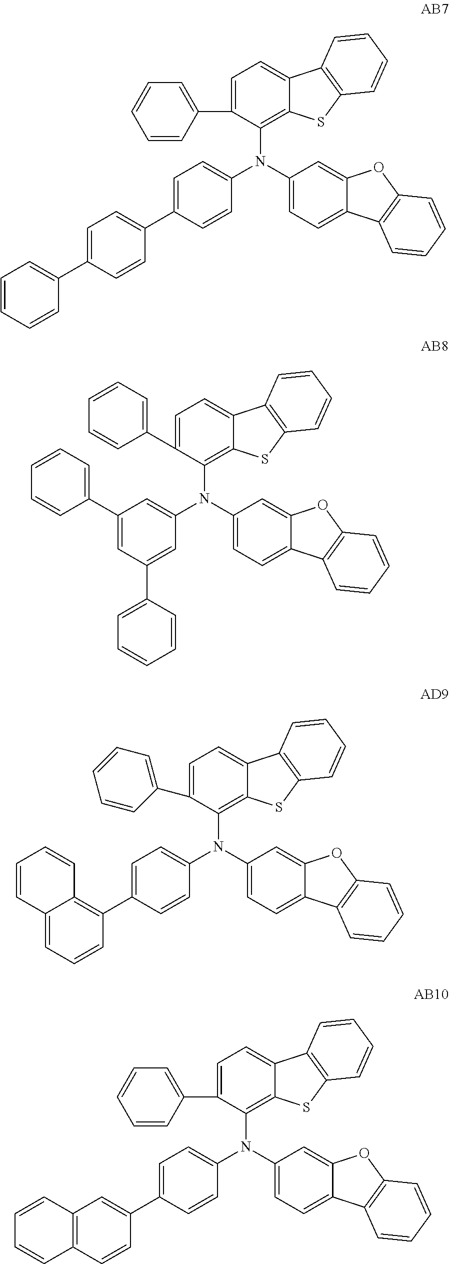 [SMILES: c1ccc(-c2ccc(-c3ccc(N(c4ccc5c(c4)oc4ccccc45)c4c(-c5ccccc5)ccc5c4sc4ccccc45)cc3)cc2)cc1 ; c1ccc(-c2cc(-c3ccccc3)cc(N(c3ccc4c(c3)oc3ccccc34)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2ccc3c(c2)oc2ccccc23)cc1 ; c1ccc(-c2ccc3c(sc4ccccc43)c2N(c2ccc(-c3ccc4ccccc4c3)cc2)c2ccc3c(c2)oc2ccccc23)cc1]

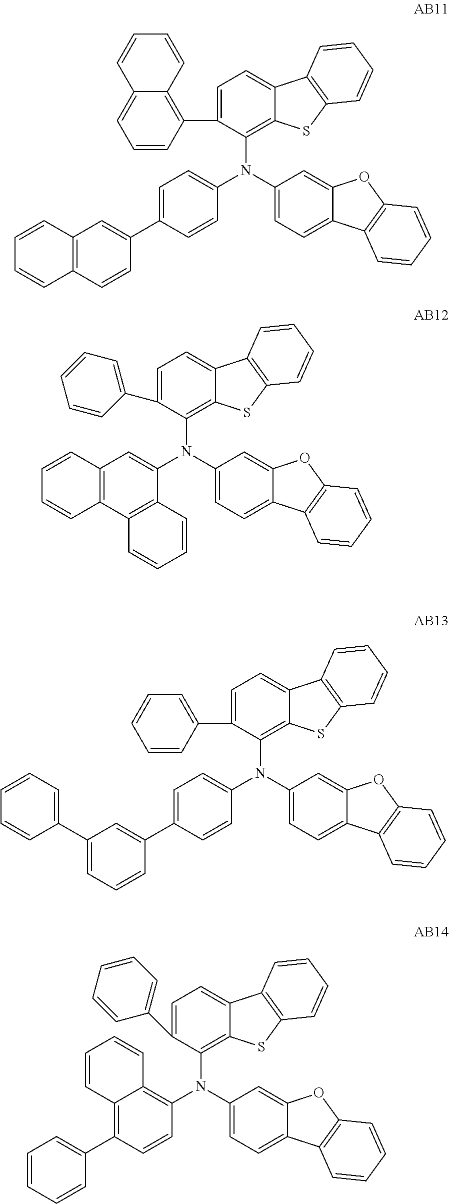 [SMILES: c1ccc2cc(-c3ccc(N(c4ccc5c(c4)oc4ccccc45)c4c(-c5cccc6ccccc56)ccc5c4sc4ccccc45)cc3)ccc2c1 ; c1ccc(-c2ccc3c(sc4ccccc43)c2N(c2ccc3c(c2)oc2ccccc23)c2cc3ccccc3c3ccccc23)cc1 ; c1ccc(-c2cccc(-c3ccc(N(c4ccc5c(c4)oc4ccccc45)c4c(-c5ccccc5)ccc5c4sc4ccccc45)cc3)c2)cc1 ; c1ccc(-c2ccc3c(sc4ccccc43)c2N(c2ccc3c(c2)oc2ccccc23)c2ccc(-c3ccccc3)c3ccccc23)cc1]

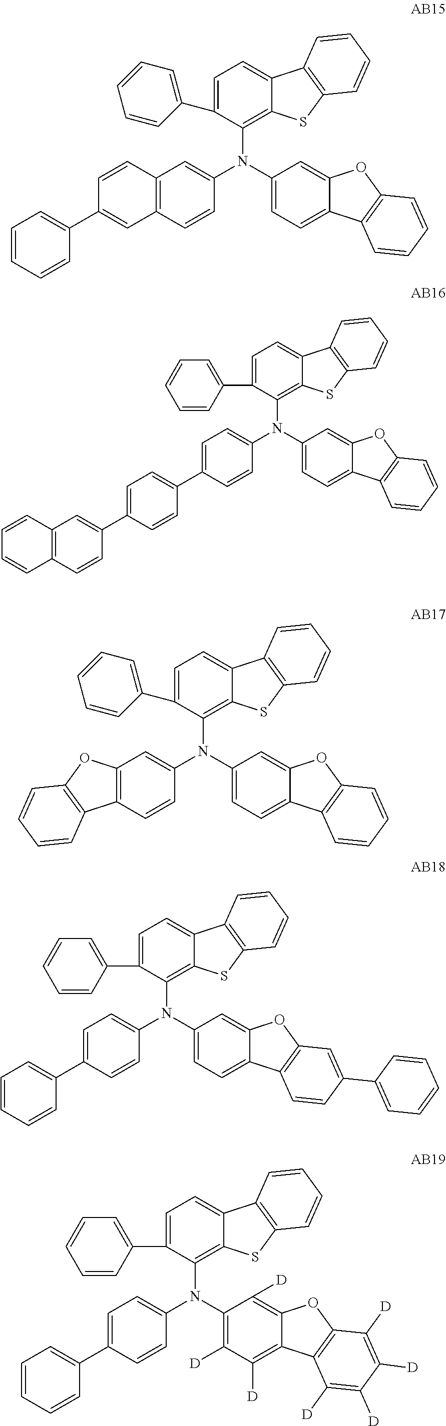 [SMILES: c1ccc(-c2ccc3cc(N(c4ccc5c(c4)oc4ccccc45)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc3c(c2)oc2ccccc23)cc1 ; c1ccc(-c2ccc3c(sc4ccccc43)c2N(c2ccc3c(c2)oc2ccccc23)c2ccc3c(c2)oc2ccccc23)cc1 ; c1ccc(-c2ccc(N(c3ccc4c(c3)oc3cc(-c5ccccc5)ccc34)c3c(-c4ccccc4)ccc4c3sc3ccccc34)cc2)cc1 ; [2H]c1c([2H])c([2H])c2c(oc3c([2H])c(N(c4ccc(-c5ccccc5)cc4)c4c(-c5ccccc5)ccc5c4sc4ccccc45)c([2H])c([2H])c32)c1[2H]]

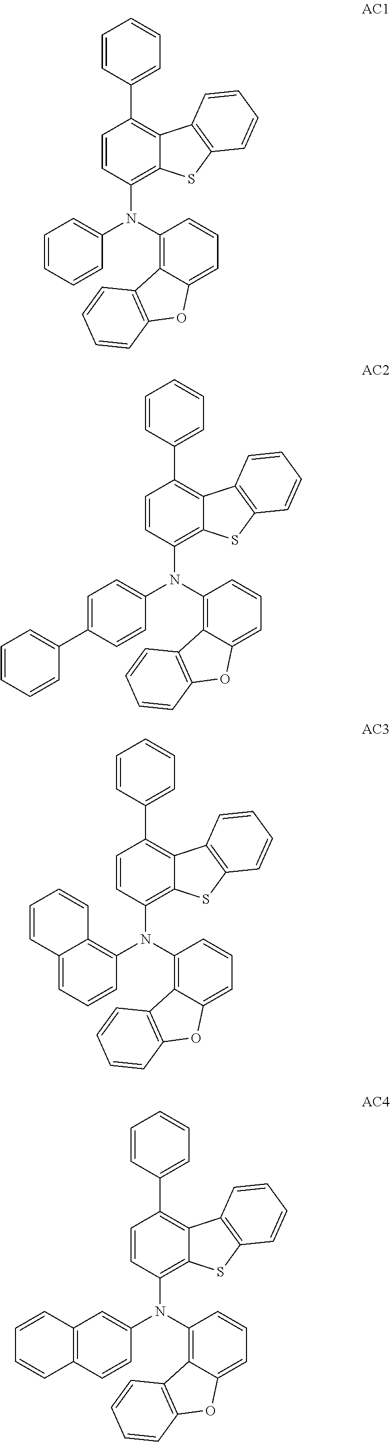

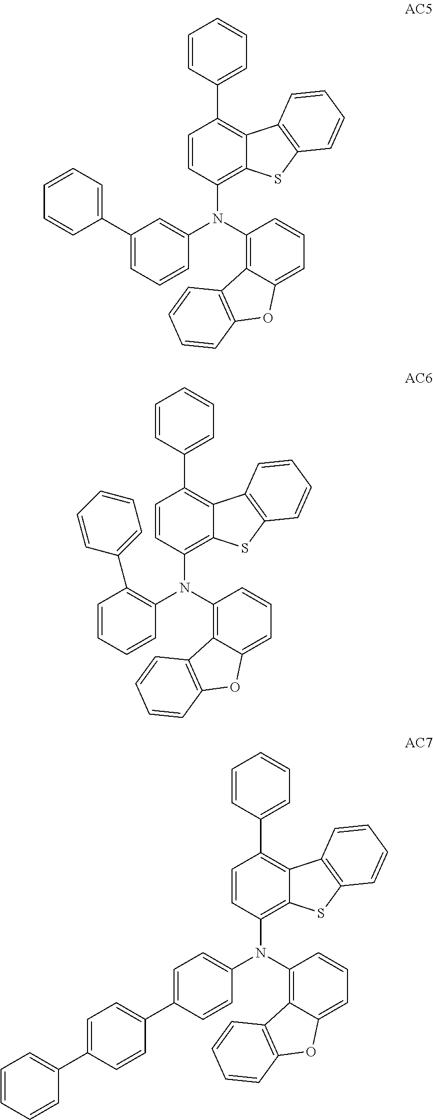

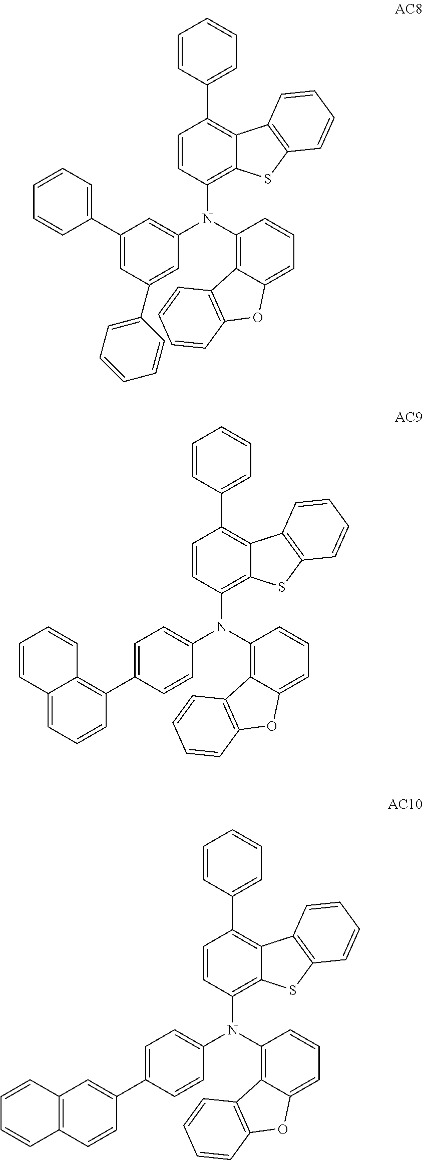 [SMILES: c1ccc(-c2cc(-c3ccccc3)cc(N(c3ccc(-c4ccccc4)c4c3sc3ccccc34)c3cccc4oc5ccccc5c34)c2)cc1 ; c1ccc(-c2ccc(N(c3ccc(-c4cccc5ccccc45)cc3)c3cccc4oc5ccccc5c34)c3sc4ccccc4c23)cc1 ; c1ccc(-c2ccc(N(c3ccc(-c4ccc5ccccc5c4)cc3)c3cccc4oc5ccccc5c34)c3sc4ccccc4c23)cc1]

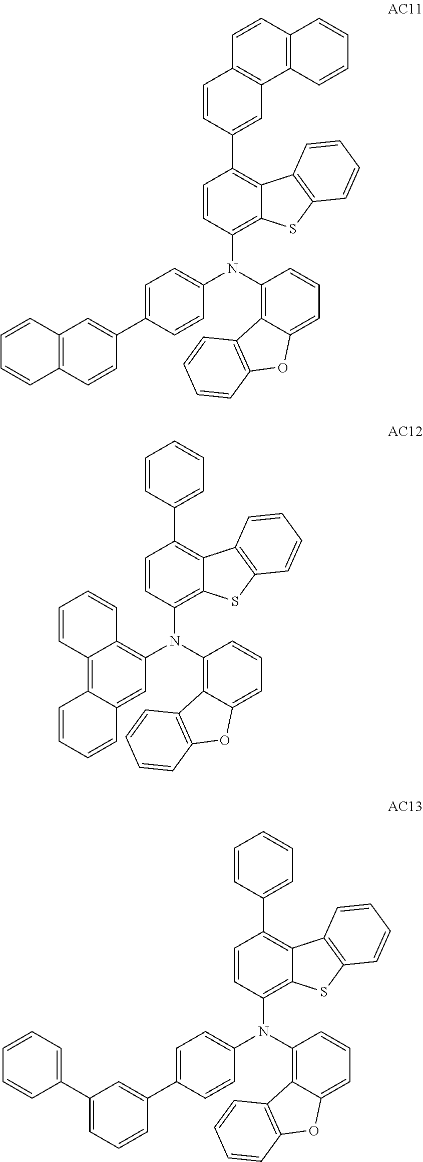 [SMILES: c1ccc2cc(-c3ccc(N(c4ccc(-c5ccc6ccc7ccccc7c6c5)c5c4sc4ccccc45)c4cccc5oc6ccccc6c45)cc3)ccc2c1 ; c1ccc(-c2ccc(N(c3cc4ccccc4c4ccccc34)c3cccc4oc5ccccc5c34)c3sc4ccccc4c23)cc1 ; c1ccc(-c2cccc(-c3ccc(N(c4ccc(-c5ccccc5)c5c4sc4ccccc45)c4cccc5oc6ccccc6c45)cc3)c2)cc1]

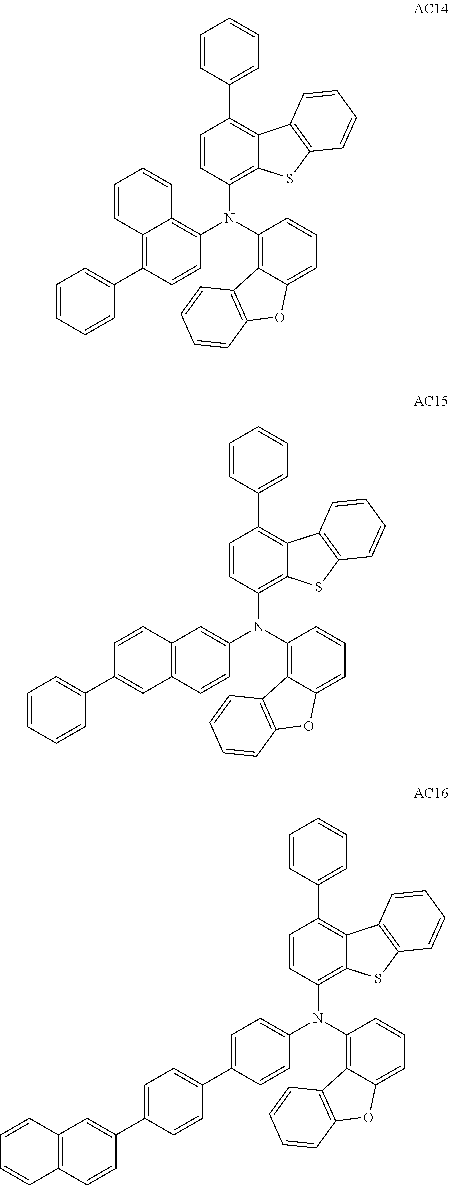 [SMILES: c1ccc(-c2ccc(N(c3ccc(-c4ccccc4)c4c3sc3ccccc34)c3cccc4oc5ccccc5c34)c3ccccc23)cc1 ; c1ccc(-c2ccc3cc(N(c4ccc(-c5ccccc5)c5c4sc4ccccc45)c4cccc5oc6ccccc6c45)ccc3c2)cc1 ; c1ccc(-c2ccc(N(c3ccc(-c4ccc(-c5ccc6ccccc6c5)cc4)cc3)c3cccc4oc5ccccc5c34)c3sc4ccccc4c23)cc1]

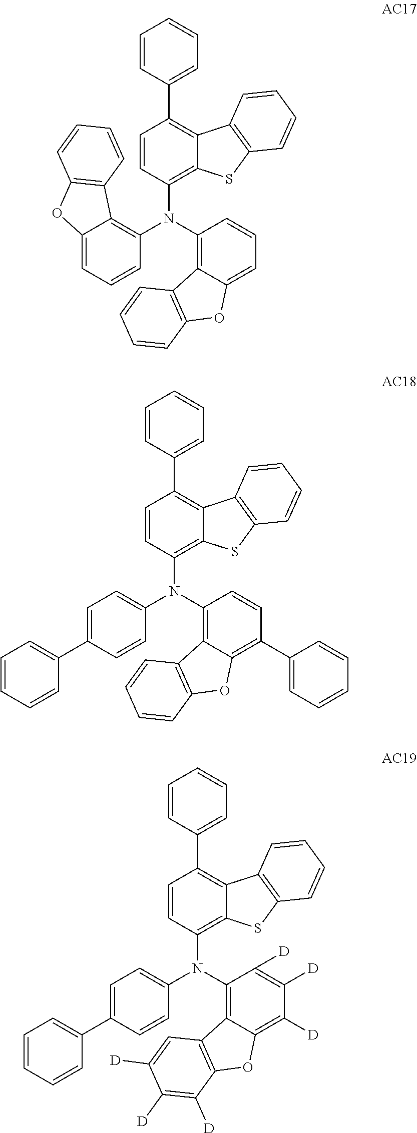

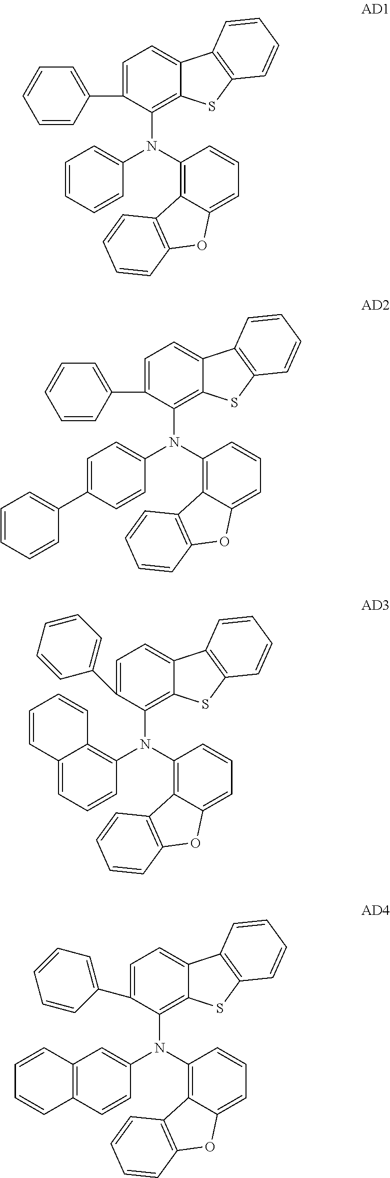 [SMILES: c1ccc(-c2ccc3c(sc4ccccc43)c2N(c2ccccc2)c2cccc3oc4ccccc4c23)cc1 ; c1ccc(-c2ccc(N(c3c(-c4ccccc4)ccc4c3sc3ccccc34)c3cccc4oc5ccccc5c34)cc2)cc1 ; c1ccc(-c2ccc3c(sc4ccccc43)c2N(c2cccc3ccccc23)c2cccc3oc4ccccc4c23)cc1 ; c1ccc(-c2ccc3c(sc4ccccc43)c2N(c2ccc3ccccc3c2)c2cccc3oc4ccccc4c23)cc1]

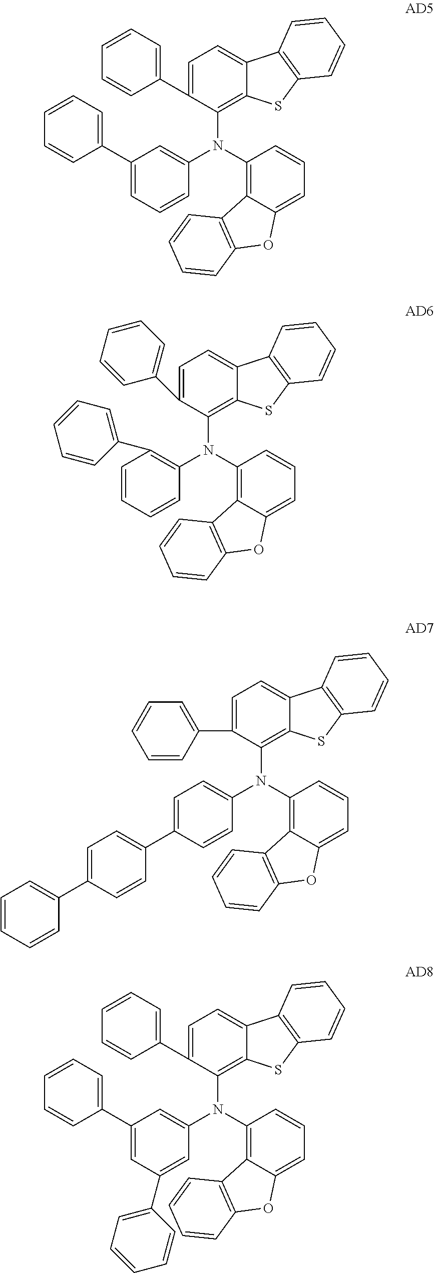 [SMILES: c1ccc(-c2cccc(N(c3c(-c4ccccc4)ccc4c3sc3ccccc34)c3cccc4oc5ccccc5c34)c2)cc1 ; c1ccc(-c2ccccc2N(c2c(-c3ccccc3)ccc3c2sc2ccccc23)c2cccc3oc4ccccc4c23)cc1 ; c1ccc(-c2ccc(-c3ccc(N(c4c(-c5ccccc5)ccc5c4sc4ccccc45)c4cccc5oc6ccccc6c45)cc3)cc2)cc1 ; c1ccc(-c2cc(-c3ccccc3)cc(N(c3c(-c4ccccc4)ccc4c3sc3ccccc34)c3cccc4oc5ccccc5c34)c2)cc1]

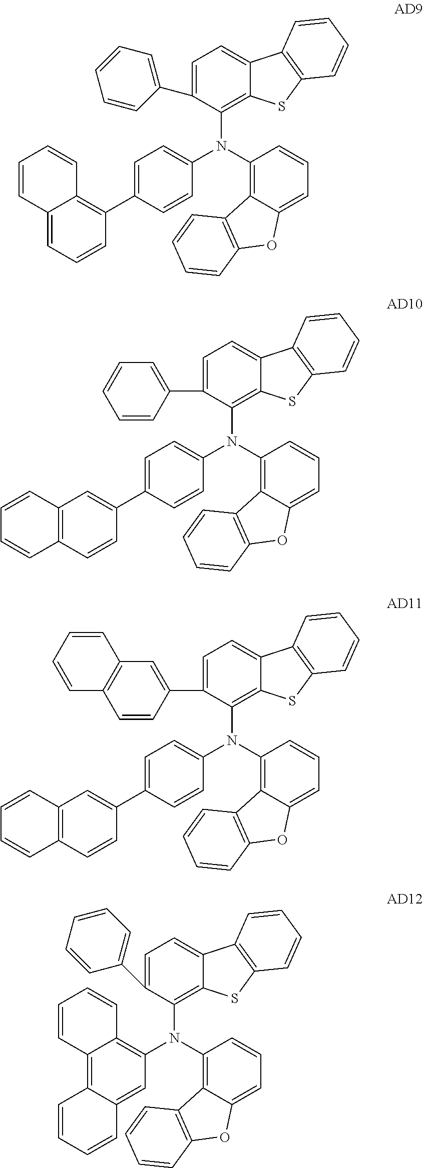 [SMILES: c1ccc(-c2ccc3c(sc4ccccc43)c2N(c2ccc(-c3cccc4ccccc34)cc2)c2cccc3oc4ccccc4c23)cc1 ; c1ccc(-c2ccc3c(sc4ccccc43)c2N(c2ccc(-c3ccc4ccccc4c3)cc2)c2cccc3oc4ccccc4c23)cc1 ; c1ccc2cc(-c3ccc(N(c4c(-c5ccc6ccccc6c5)ccc5c4sc4ccccc45)c4cccc5oc6ccccc6c45)cc3)ccc2c1 ; c1ccc(-c2ccc3c(sc4ccccc43)c2N(c2cc3ccccc3c3ccccc23)c2cccc3oc4ccccc4c23)cc1]

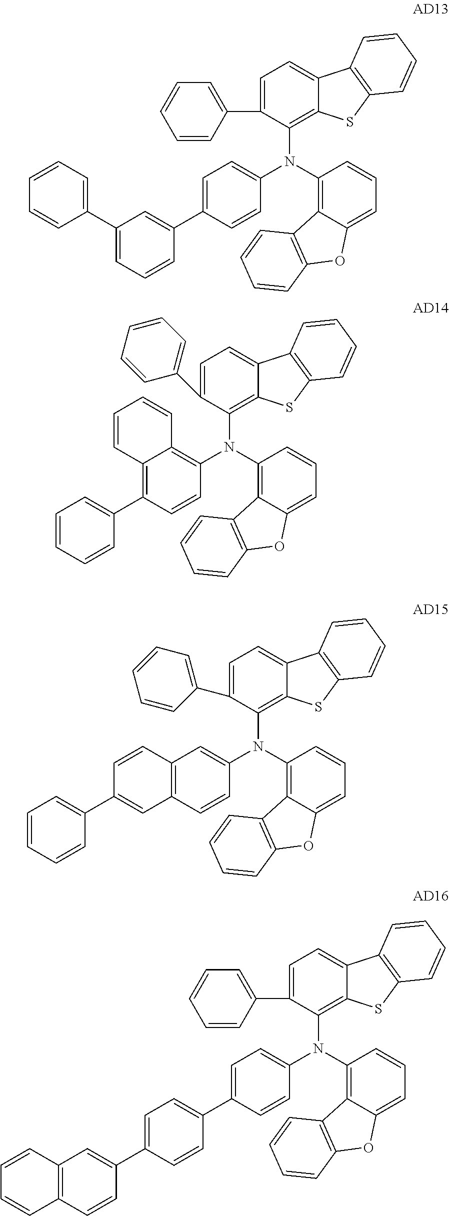

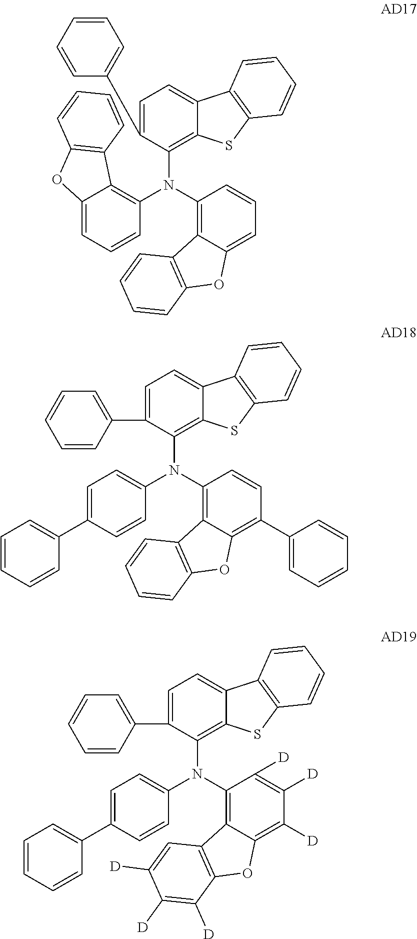 [SMILES: c1ccc(-c2ccc3c(sc4ccccc43)c2N(c2cccc3oc4ccccc4c23)c2cccc3oc4ccccc4c23)cc1 ; c1ccc(-c2ccc(N(c3c(-c4ccccc4)ccc4c3sc3ccccc34)c3ccc(-c4ccccc4)c4oc5ccccc5c34)cc2)cc1 ; [2H]c1cc2c(oc3c([2H])c([2H])c([2H])c(N(c4ccc(-c5ccccc5)cc4)c4c(-c5ccccc5)ccc5c4sc4ccccc45)c32)c([2H])c1[2H]]

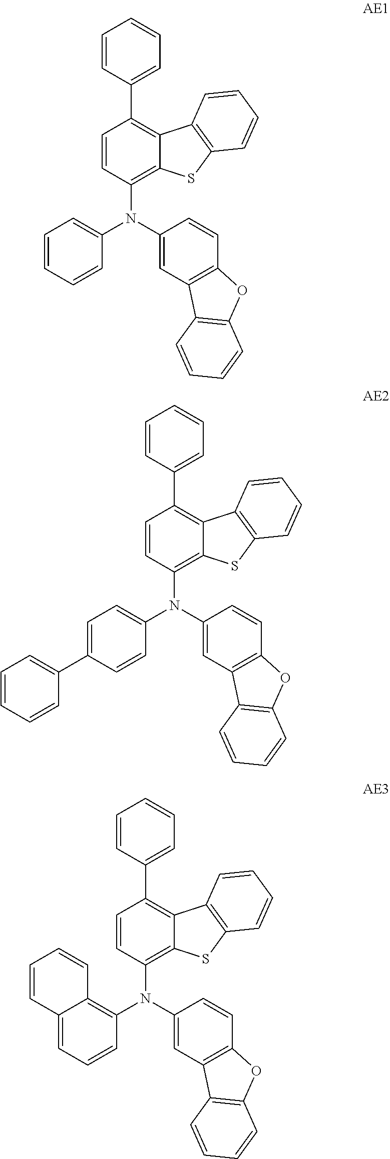

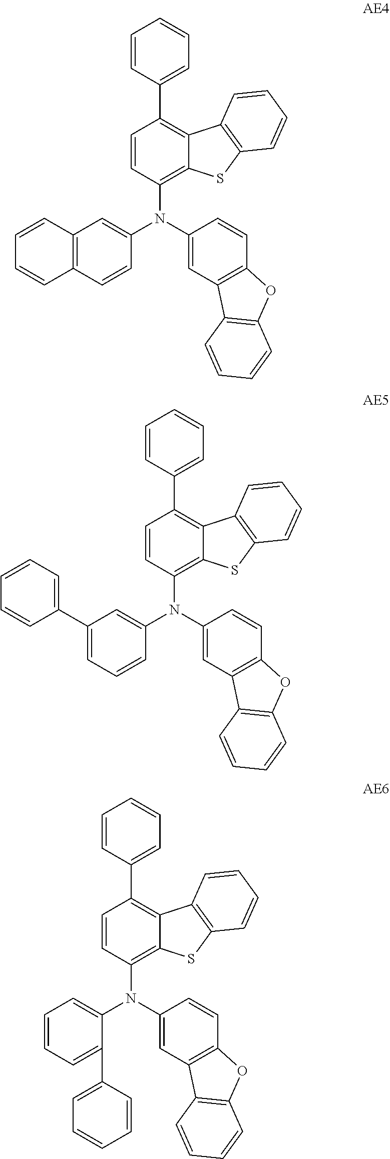 [SMILES: c1ccc(-c2ccc(N(c3ccc4ccccc4c3)c3ccc4oc5ccccc5c4c3)c3sc4ccccc4c23)cc1 ; c1ccc(-c2cccc(N(c3ccc4oc5ccccc5c4c3)c3ccc(-c4ccccc4)c4c3sc3ccccc34)c2)cc1 ; c1ccc(-c2ccccc2N(c2ccc3oc4ccccc4c3c2)c2ccc(-c3ccccc3)c3c2sc2ccccc23)cc1]

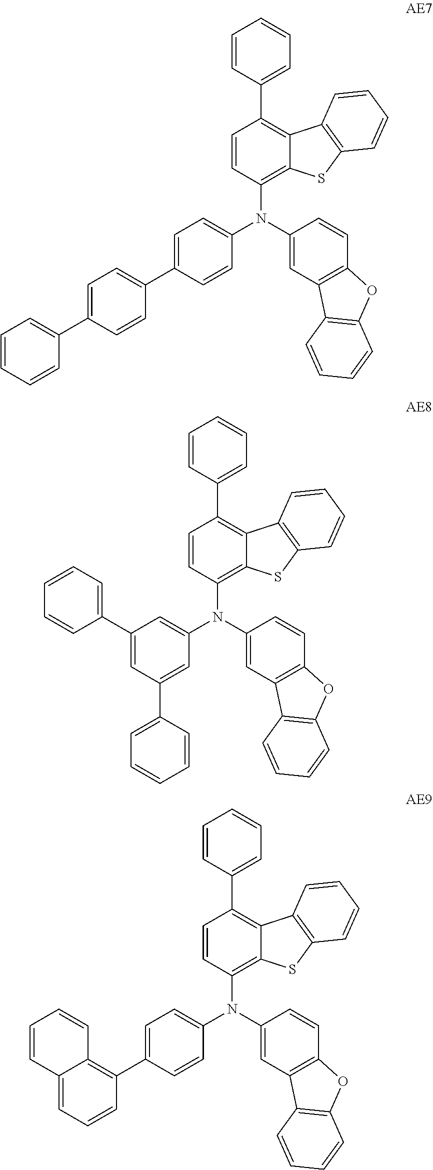 [SMILES: c1ccc(-c2ccc(-c3ccc(N(c4ccc5oc6ccccc6c5c4)c4ccc(-c5ccccc5)c5c4sc4ccccc45)cc3)cc2)cc1 ; c1ccc(-c2cc(-c3ccccc3)cc(N(c3ccc4oc5ccccc5c4c3)c3ccc(-c4ccccc4)c4c3sc3ccccc34)c2)cc1 ; c1ccc(-c2ccc(N(c3ccc(-c4cccc5ccccc45)cc3)c3ccc4oc5ccccc5c4c3)c3sc4ccccc4c23)cc1]

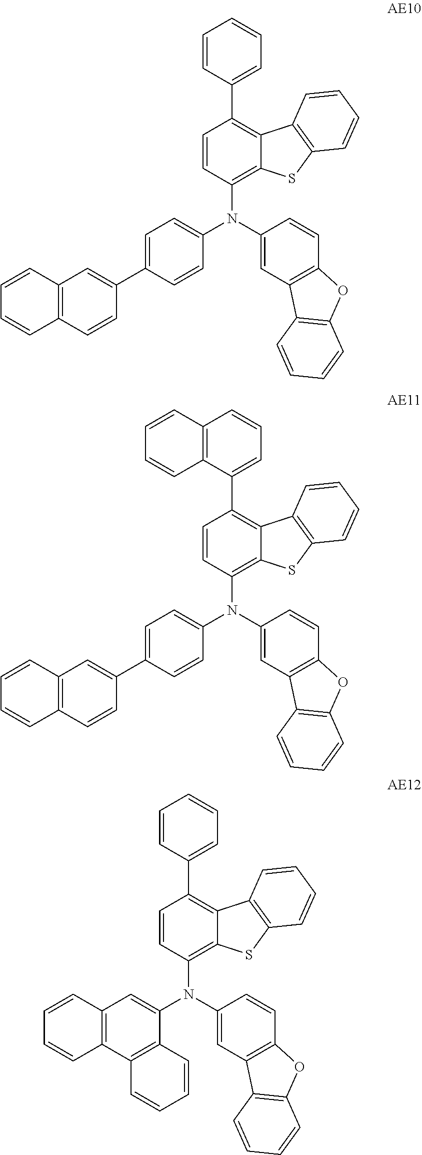 [SMILES: c1ccc(-c2ccc(N(c3ccc(-c4ccc5ccccc5c4)cc3)c3ccc4oc5ccccc5c4c3)c3sc4ccccc4c23)cc1 ; c1ccc2cc(-c3ccc(N(c4ccc5oc6ccccc6c5c4)c4ccc(-c5cccc6ccccc56)c5c4sc4ccccc45)cc3)ccc2c1 ; c1ccc(-c2ccc(N(c3ccc4oc5ccccc5c4c3)c3cc4ccccc4c4ccccc34)c3sc4ccccc4c23)cc1]

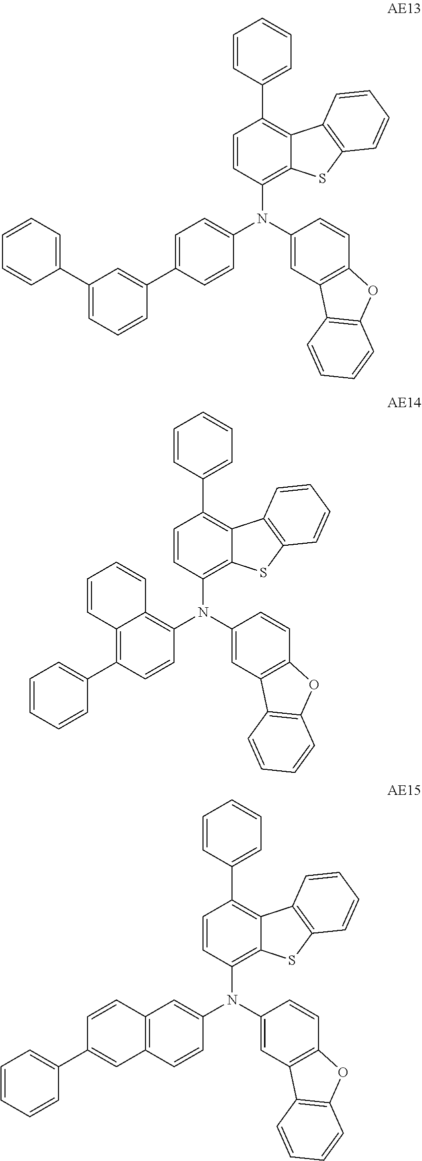

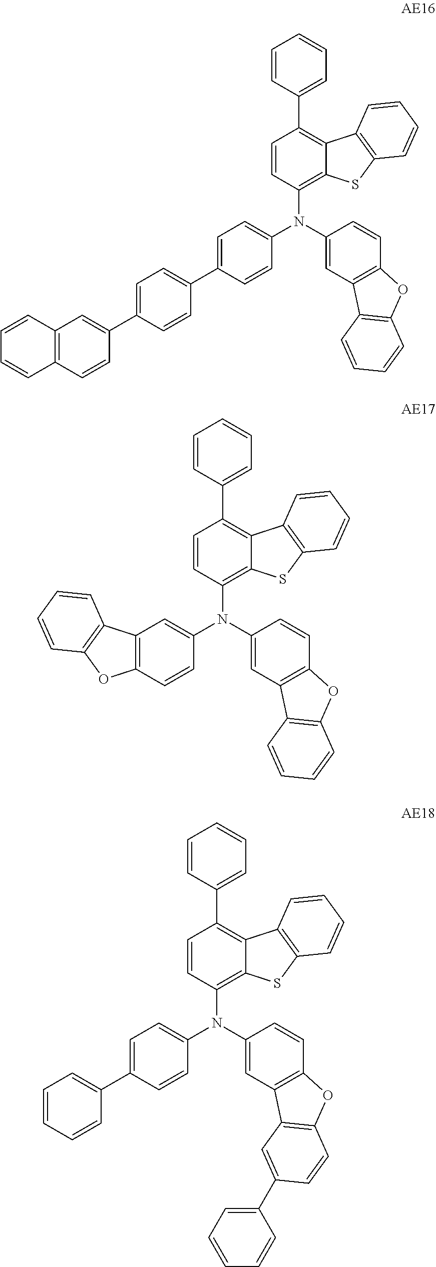 [SMILES: c1ccc(-c2ccc(N(c3ccc(-c4ccc(-c5ccc6ccccc6c5)cc4)cc3)c3ccc4oc5ccccc5c4c3)c3sc4ccccc4c23)cc1 ; c1ccc(-c2ccc(N(c3ccc4oc5ccccc5c4c3)c3ccc4oc5ccccc5c4c3)c3sc4ccccc4c23)cc1 ; c1ccc(-c2ccc(N(c3ccc4oc5ccc(-c6ccccc6)cc5c4c3)c3ccc(-c4ccccc4)c4c3sc3ccccc34)cc2)cc1]

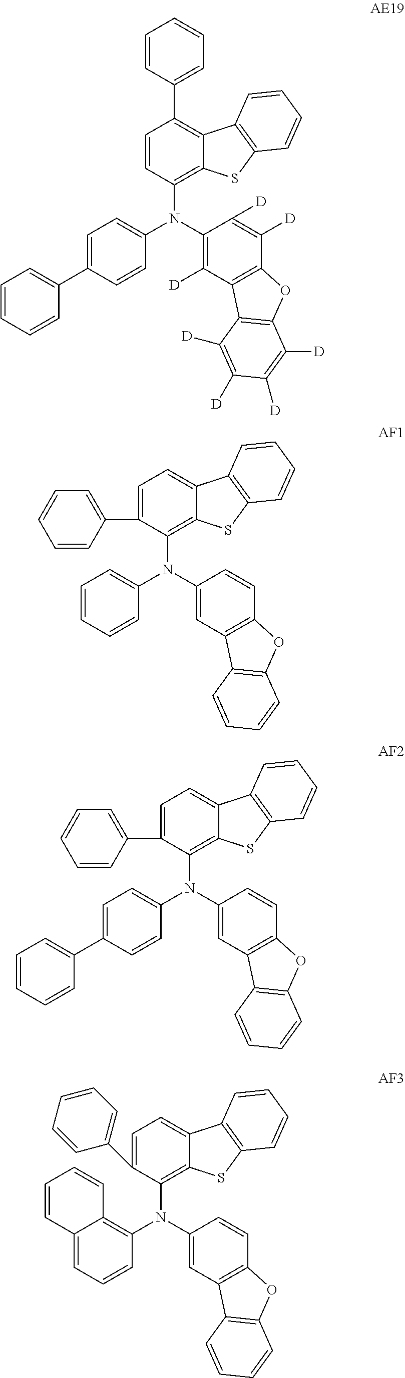

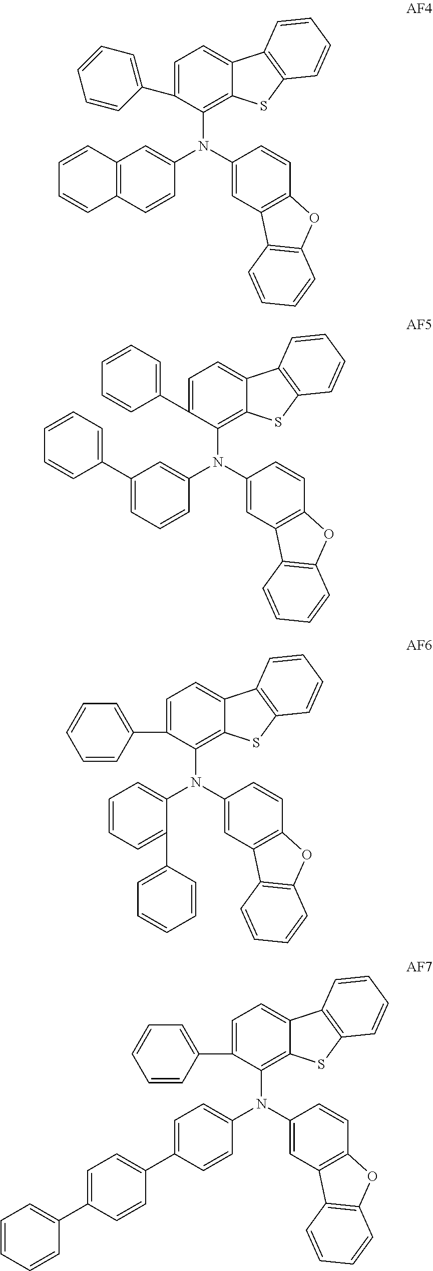

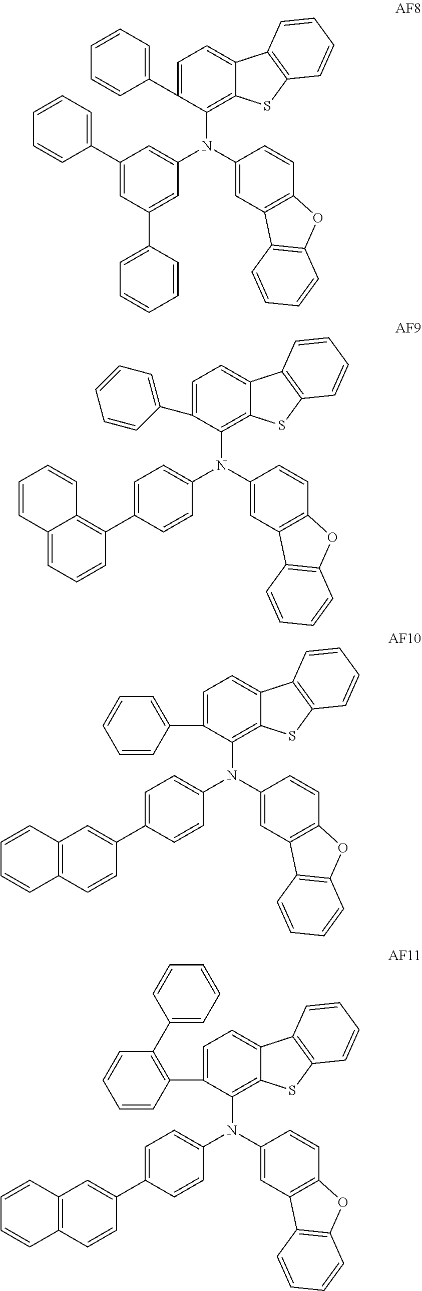

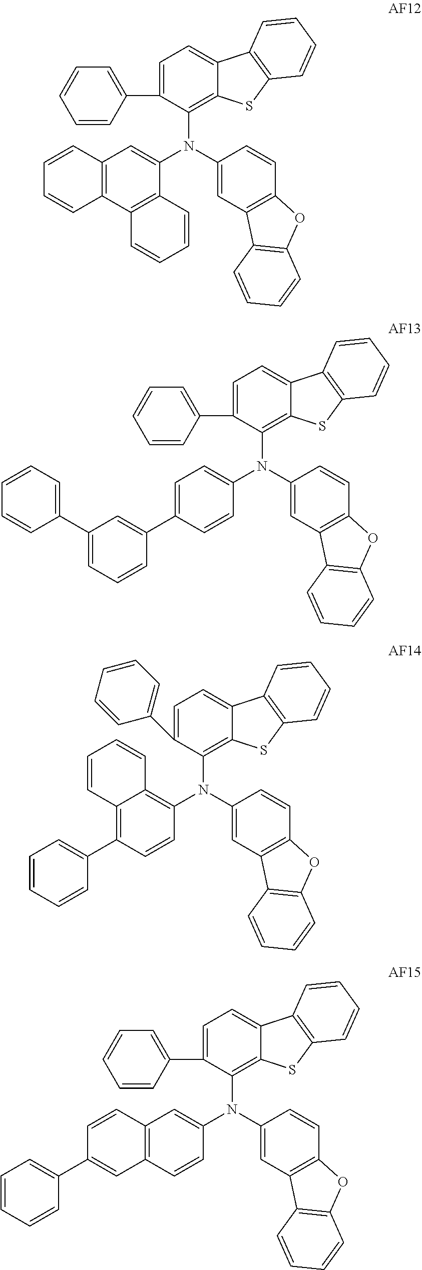

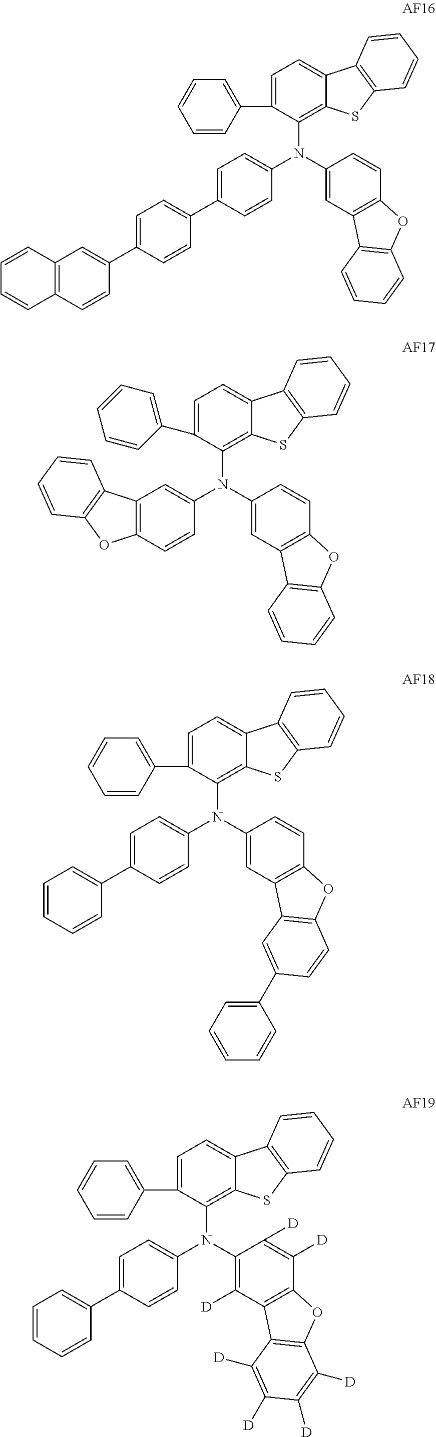

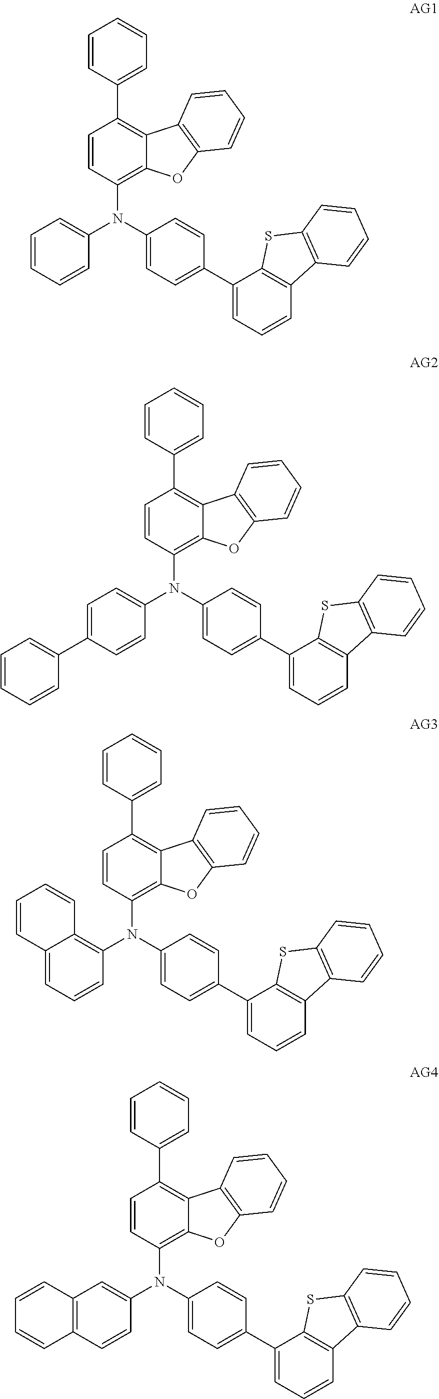 [SMILES: c1ccc(-c2ccc(N(c3ccccc3)c3ccc(-c4cccc5c4sc4ccccc45)cc3)c3oc4ccccc4c23)cc1 ; c1ccc(-c2ccc(N(c3ccc(-c4cccc5c4sc4ccccc45)cc3)c3ccc(-c4ccccc4)c4c3oc3ccccc34)cc2)cc1 ; c1ccc(-c2ccc(N(c3ccc(-c4cccc5c4sc4ccccc45)cc3)c3cccc4ccccc34)c3oc4ccccc4c23)cc1 ; c1ccc(-c2ccc(N(c3ccc(-c4cccc5c4sc4ccccc45)cc3)c3ccc4ccccc4c3)c3oc4ccccc4c23)cc1]

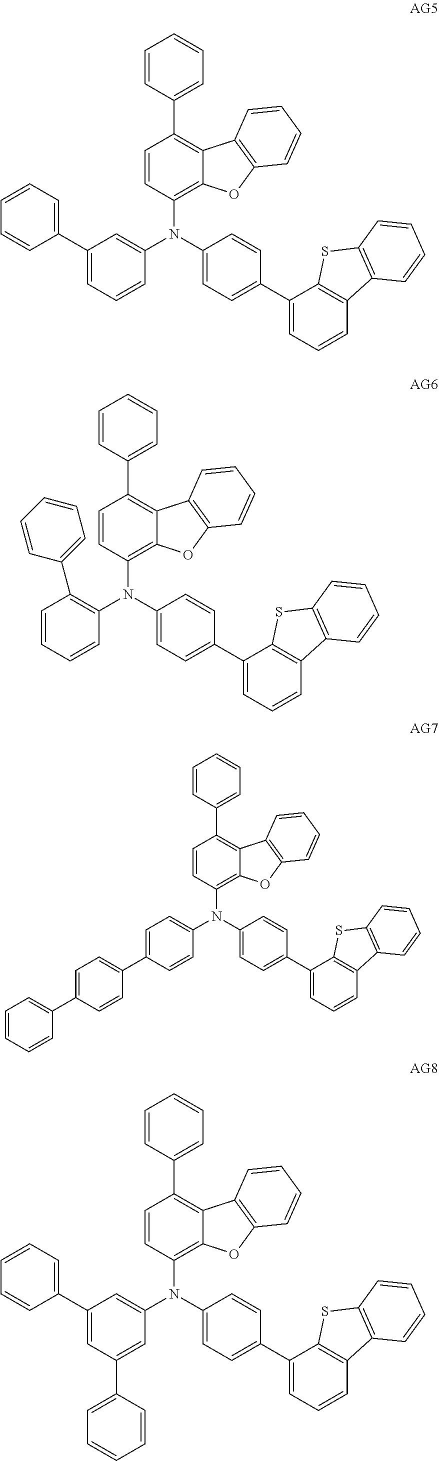 [SMILES: c1ccc(-c2cccc(N(c3ccc(-c4cccc5c4sc4ccccc45)cc3)c3ccc(-c4ccccc4)c4c3oc3ccccc34)c2)cc1 ; c1ccc(-c2ccccc2N(c2ccc(-c3cccc4c3sc3ccccc34)cc2)c2ccc(-c3ccccc3)c3c2oc2ccccc23)cc1 ; c1ccc(-c2ccc(-c3ccc(N(c4ccc(-c5cccc6c5sc5ccccc56)cc4)c4ccc(-c5ccccc5)c5c4oc4ccccc45)cc3)cc2)cc1 ; c1ccc(-c2cc(-c3ccccc3)cc(N(c3ccc(-c4cccc5c4sc4ccccc45)cc3)c3ccc(-c4ccccc4)c4c3oc3ccccc34)c2)cc1]

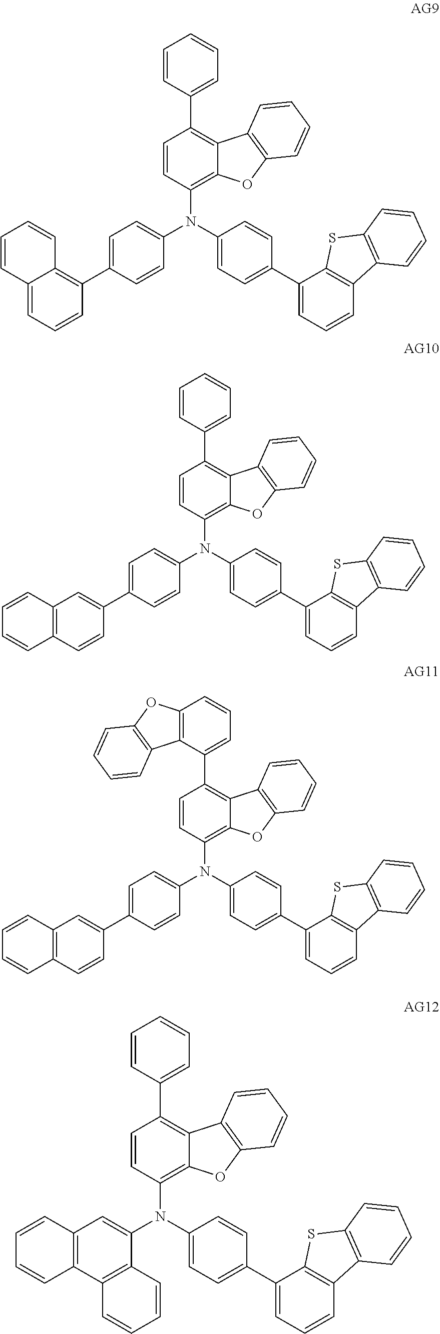 [SMILES: c1ccc(-c2ccc(N(c3ccc(-c4cccc5ccccc45)cc3)c3ccc(-c4cccc5c4sc4ccccc45)cc3)c3oc4ccccc4c23)cc1 ; c1ccc(-c2ccc(N(c3ccc(-c4ccc5ccccc5c4)cc3)c3ccc(-c4cccc5c4sc4ccccc45)cc3)c3oc4ccccc4c23)cc1 ; c1ccc2cc(-c3ccc(N(c4ccc(-c5cccc6c5sc5ccccc56)cc4)c4ccc(-c5cccc6oc7ccccc7c56)c5c4oc4ccccc45)cc3)ccc2c1 ; c1ccc(-c2ccc(N(c3ccc(-c4cccc5c4sc4ccccc45)cc3)c3cc4ccccc4c4ccccc34)c3oc4ccccc4c23)cc1]

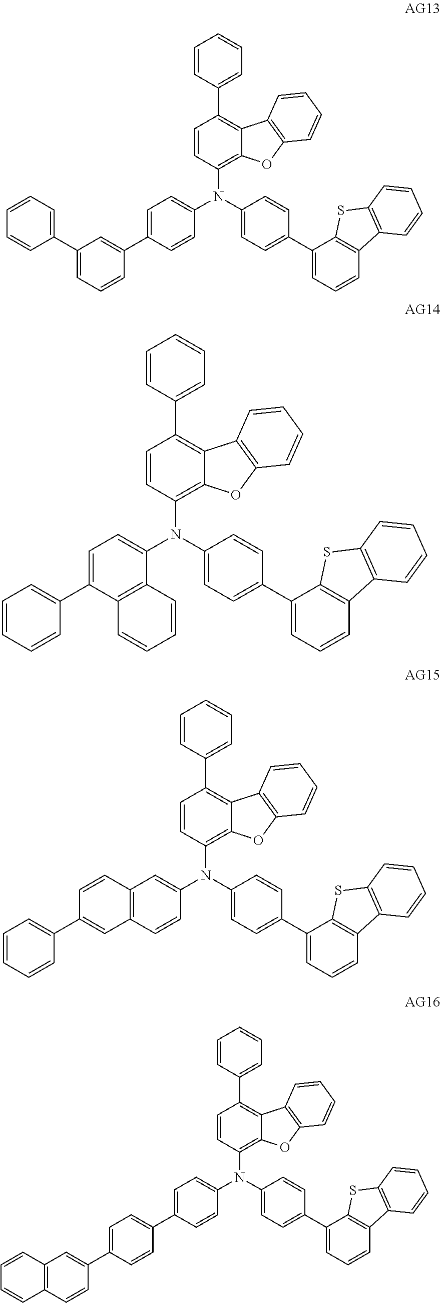 [SMILES: c1ccc(-c2cccc(-c3ccc(N(c4ccc(-c5cccc6c5sc5ccccc56)cc4)c4ccc(-c5ccccc5)c5c4oc4ccccc45)cc3)c2)cc1 ; c1ccc(-c2ccc(N(c3ccc(-c4cccc5c4sc4ccccc45)cc3)c3ccc(-c4ccccc4)c4c3oc3ccccc34)c3ccccc23)cc1 ; c1ccc(-c2ccc3cc(N(c4ccc(-c5cccc6c5sc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4cccc5c4sc4ccccc45)cc3)c3oc4ccccc4c23)cc1]

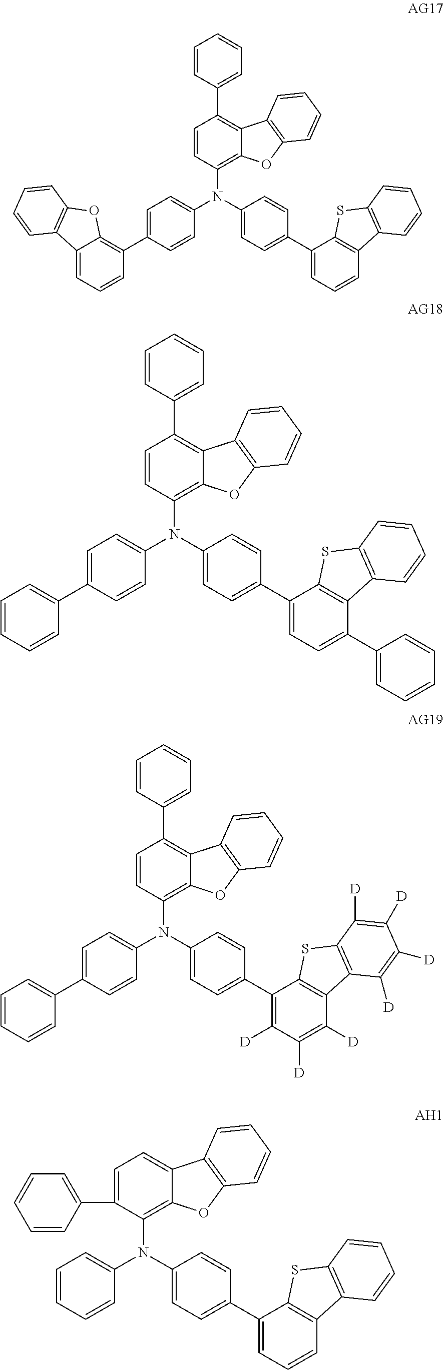 [SMILES: c1ccc(-c2ccc(N(c3ccc(-c4cccc5c4oc4ccccc45)cc3)c3ccc(-c4cccc5c4sc4ccccc45)cc3)c3oc4ccccc4c23)cc1 ; c1ccc(-c2ccc(N(c3ccc(-c4ccc(-c5ccccc5)c5c4sc4ccccc45)cc3)c3ccc(-c4ccccc4)c4c3oc3ccccc34)cc2)cc1 ; [2H]c1c([2H])c([2H])c2c(sc3c(-c4ccc(N(c5ccc(-c6ccccc6)cc5)c5ccc(-c6ccccc6)c6c5oc5ccccc56)cc4)c([2H])c([2H])c([2H])c32)c1[2H] ; c1ccc(-c2ccc3c(oc4ccccc43)c2N(c2ccccc2)c2ccc(-c3cccc4c3sc3ccccc34)cc2)cc1]

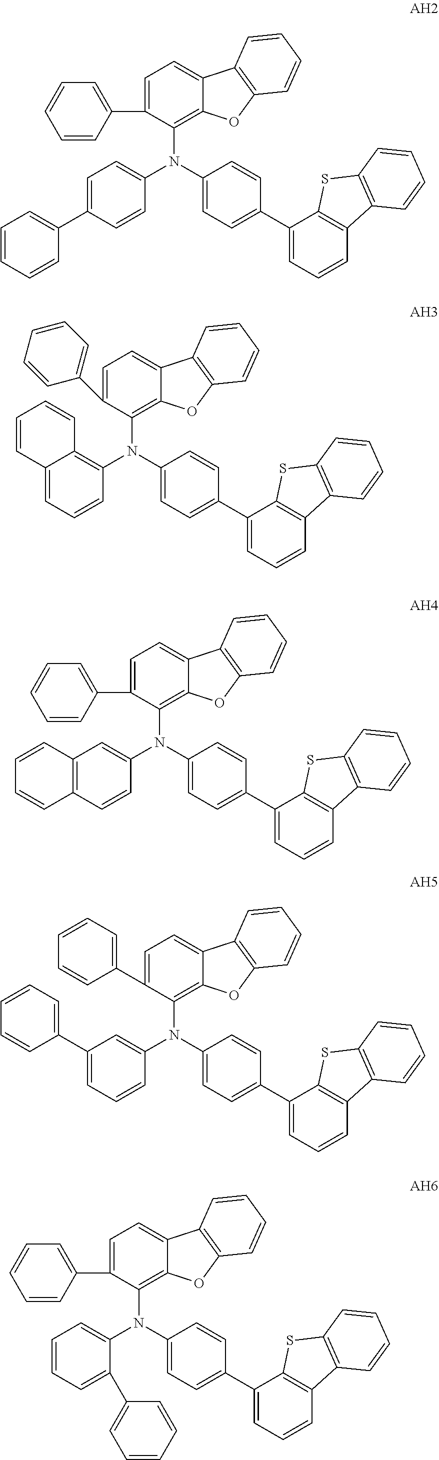 [SMILES: c1ccc(-c2ccc(N(c3ccc(-c4cccc5c4sc4ccccc45)cc3)c3c(-c4ccccc4)ccc4c3oc3ccccc34)cc2)cc1 ; c1ccc(-c2ccc3c(oc4ccccc43)c2N(c2ccc(-c3cccc4c3sc3ccccc34)cc2)c2cccc3ccccc23)cc1 ; c1ccc(-c2ccc3c(oc4ccccc43)c2N(c2ccc(-c3cccc4c3sc3ccccc34)cc2)c2ccc3ccccc3c2)cc1 ; c1ccc(-c2cccc(N(c3ccc(-c4cccc5c4sc4ccccc45)cc3)c3c(-c4ccccc4)ccc4c3oc3ccccc34)c2)cc1 ; c1ccc(-c2ccccc2N(c2ccc(-c3cccc4c3sc3ccccc34)cc2)c2c(-c3ccccc3)ccc3c2oc2ccccc23)cc1]

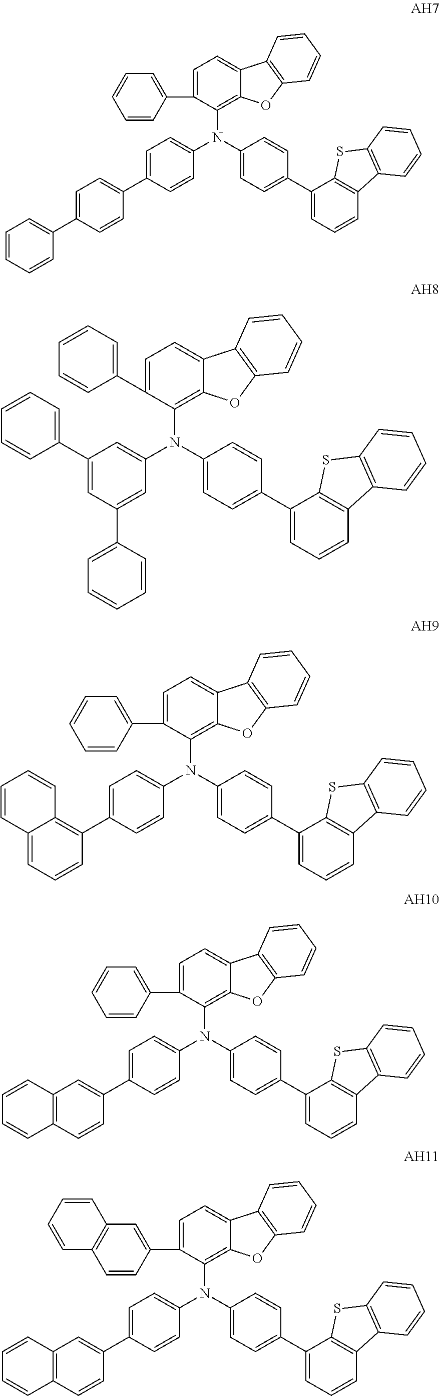 [SMILES: c1ccc(-c2ccc(-c3ccc(N(c4ccc(-c5cccc6c5sc5ccccc56)cc4)c4c(-c5ccccc5)ccc5c4oc4ccccc45)cc3)cc2)cc1 ; c1ccc(-c2cc(-c3ccccc3)cc(N(c3ccc(-c4cccc5c4sc4ccccc45)cc3)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc(-c3cccc4c3sc3ccccc34)cc2)cc1 ; c1ccc(-c2ccc3c(oc4ccccc43)c2N(c2ccc(-c3ccc4ccccc4c3)cc2)c2ccc(-c3cccc4c3sc3ccccc34)cc2)cc1 ; c1ccc2cc(-c3ccc(N(c4ccc(-c5cccc6c5sc5ccccc56)cc4)c4c(-c5ccc6ccccc6c5)ccc5c4oc4ccccc45)cc3)ccc2c1]

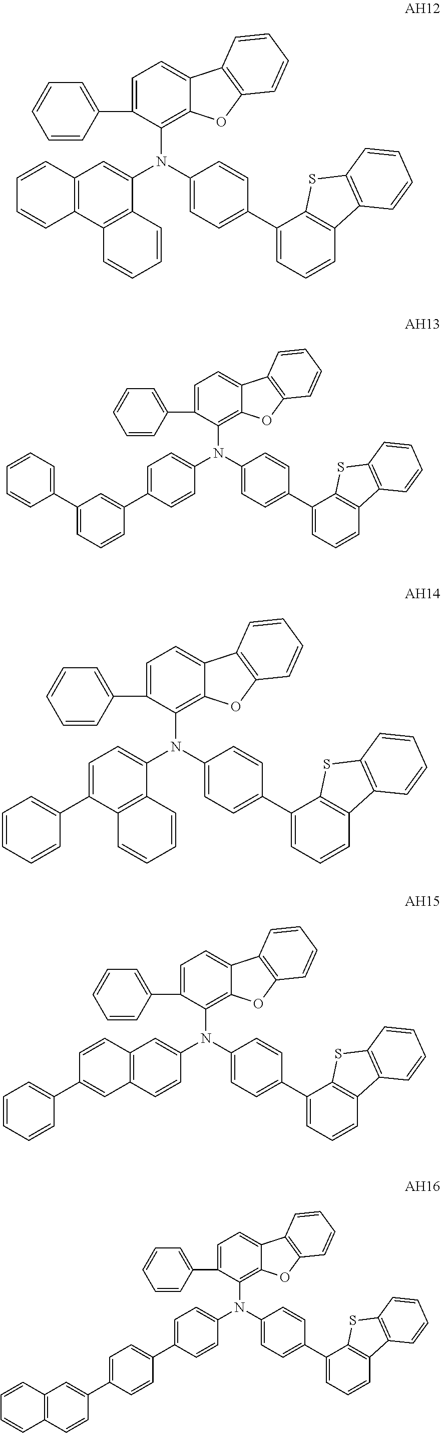 [SMILES: c1ccc(-c2ccc3c(oc4ccccc43)c2N(c2ccc(-c3cccc4c3sc3ccccc34)cc2)c2cc3ccccc3c3ccccc23)cc1 ; c1ccc(-c2cccc(-c3ccc(N(c4ccc(-c5cccc6c5sc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3cccc4c3sc3ccccc34)cc2)c2ccc(-c3ccccc3)c3ccccc23)cc1 ; c1ccc(-c2ccc3cc(N(c4ccc(-c5cccc6c5sc5ccccc56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4c3sc3ccccc34)cc2)cc1]

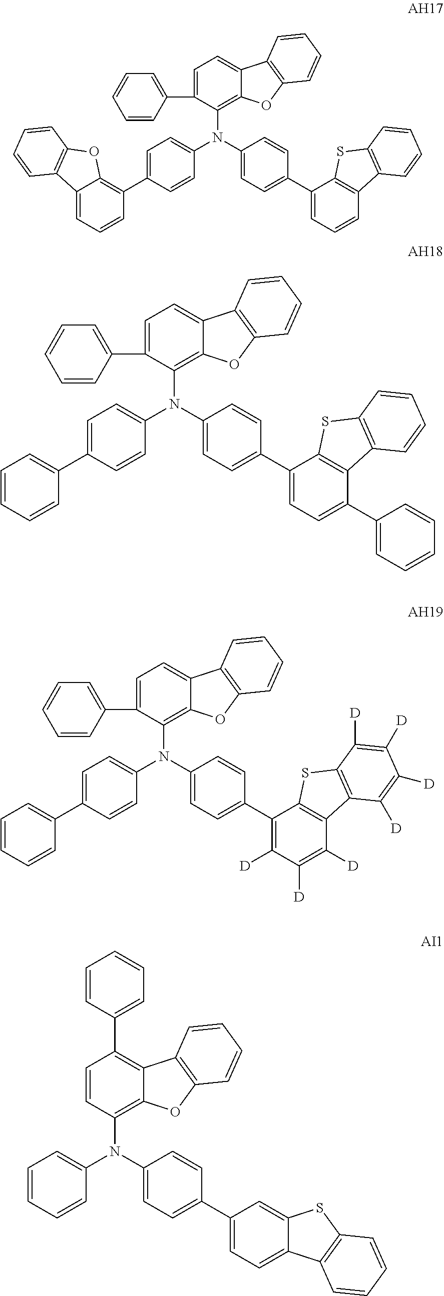 [SMILES: c1ccc(-c2ccc3c(oc4ccccc43)c2N(c2ccc(-c3cccc4c3oc3ccccc34)cc2)c2ccc(-c3cccc4c3sc3ccccc34)cc2)cc1 ; c1ccc(-c2ccc(N(c3ccc(-c4ccc(-c5ccccc5)c5c4sc4ccccc45)cc3)c3c(-c4ccccc4)ccc4c3oc3ccccc34)cc2)cc1 ; [2H]c1c([2H])c([2H])c2c(sc3c(-c4ccc(N(c5ccc(-c6ccccc6)cc5)c5c(-c6ccccc6)ccc6c5oc5ccccc56)cc4)c([2H])c([2H])c([2H])c32)c1[2H] ; c1ccc(-c2ccc(N(c3ccccc3)c3ccc(-c4ccc5c(c4)sc4ccccc45)cc3)c3oc4ccccc4c23)cc1]

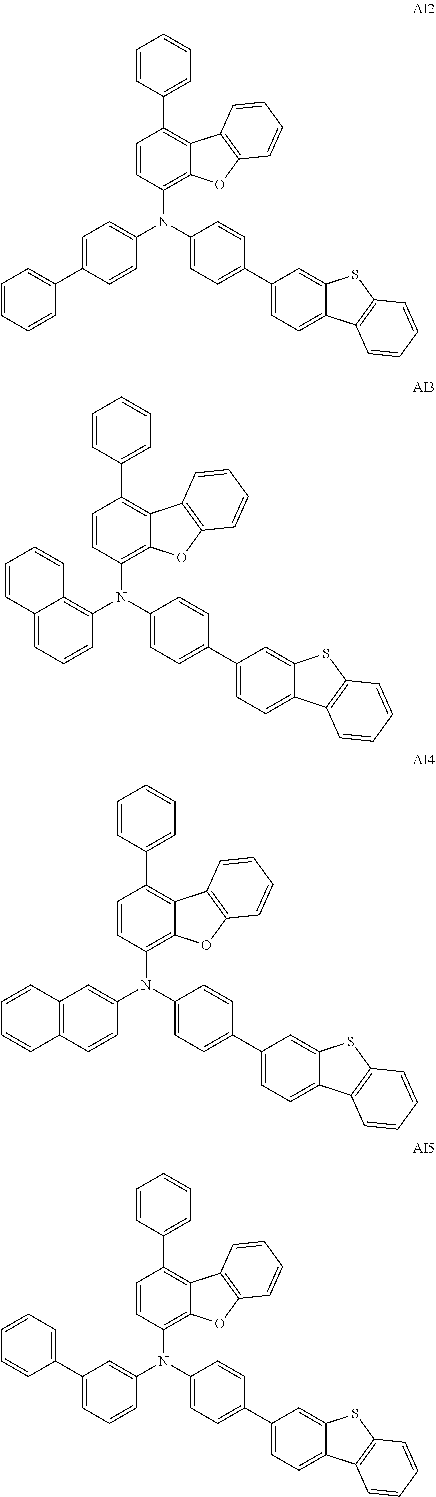 [SMILES: c1ccc(-c2ccc(N(c3ccc(-c4ccc5c(c4)sc4ccccc45)cc3)c3ccc(-c4ccccc4)c4c3oc3ccccc34)cc2)cc1 ; c1ccc(-c2ccc(N(c3ccc(-c4ccc5c(c4)sc4ccccc45)cc3)c3cccc4ccccc34)c3oc4ccccc4c23)cc1 ; c1ccc(-c2ccc(N(c3ccc(-c4ccc5c(c4)sc4ccccc45)cc3)c3ccc4ccccc4c3)c3oc4ccccc4c23)cc1 ; c1ccc(-c2cccc(N(c3ccc(-c4ccc5c(c4)sc4ccccc45)cc3)c3ccc(-c4ccccc4)c4c3oc3ccccc34)c2)cc1]

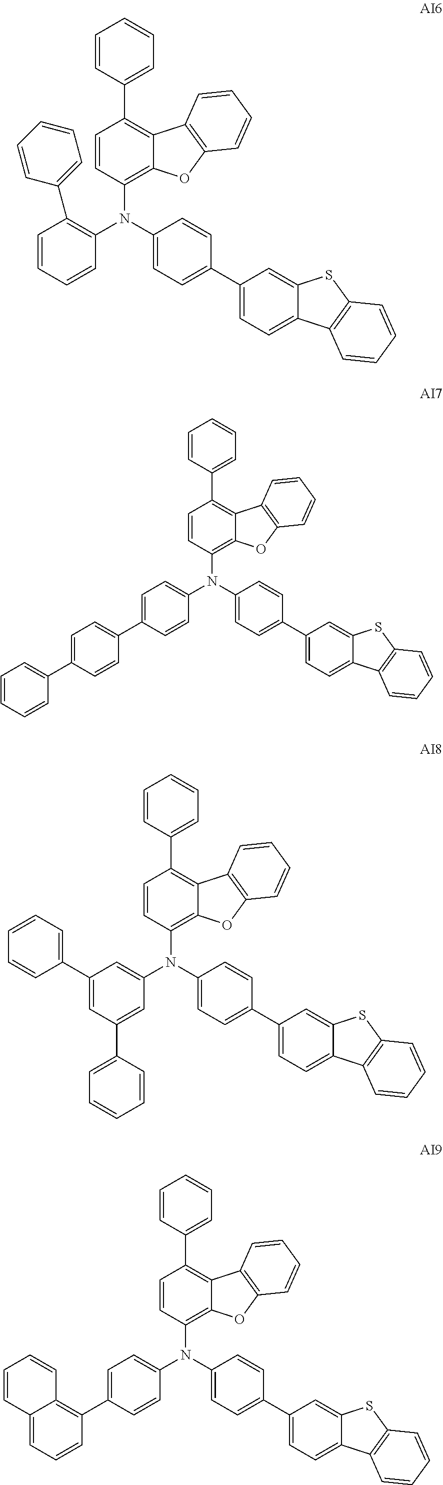 [SMILES: c1ccc(-c2ccccc2N(c2ccc(-c3ccc4c(c3)sc3ccccc34)cc2)c2ccc(-c3ccccc3)c3c2oc2ccccc23)cc1 ; c1ccc(-c2ccc(-c3ccc(N(c4ccc(-c5ccc6c(c5)sc5ccccc56)cc4)c4ccc(-c5ccccc5)c5c4oc4ccccc45)cc3)cc2)cc1 ; c1ccc(-c2cc(-c3ccccc3)cc(N(c3ccc(-c4ccc5c(c4)sc4ccccc45)cc3)c3ccc(-c4ccccc4)c4c3oc3ccccc34)c2)cc1 ; c1ccc(-c2ccc(N(c3ccc(-c4ccc5c(c4)sc4ccccc45)cc3)c3ccc(-c4cccc5ccccc45)cc3)c3oc4ccccc4c23)cc1]

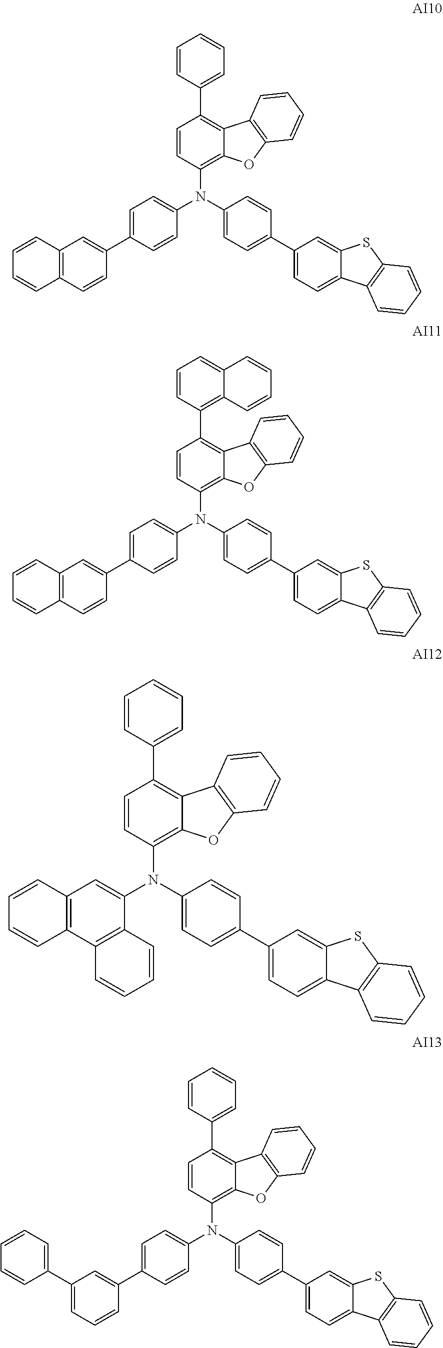 [SMILES: c1ccc(-c2ccc(N(c3ccc(-c4ccc5ccccc5c4)cc3)c3ccc(-c4ccc5c(c4)sc4ccccc45)cc3)c3oc4ccccc4c23)cc1 ; c1ccc2cc(-c3ccc(N(c4ccc(-c5ccc6c(c5)sc5ccccc56)cc4)c4ccc(-c5cccc6ccccc56)c5c4oc4ccccc45)cc3)ccc2c1 ; c1ccc(-c2ccc(N(c3ccc(-c4ccc5c(c4)sc4ccccc45)cc3)c3cc4ccccc4c4ccccc34)c3oc4ccccc4c23)cc1 ; c1ccc(-c2cccc(-c3ccc(N(c4ccc(-c5ccc6c(c5)sc5ccccc56)cc4)c4ccc(-c5ccccc5)c5c4oc4ccccc45)cc3)c2)cc1]

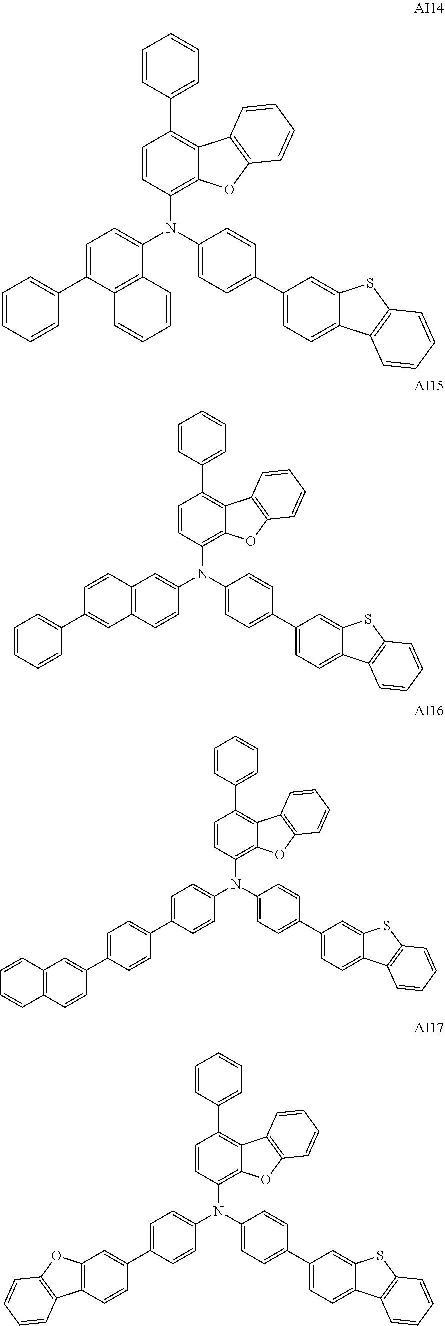 [SMILES: c1ccc(-c2ccc(N(c3ccc(-c4ccc5c(c4)sc4ccccc45)cc3)c3ccc(-c4ccccc4)c4c3oc3ccccc34)c3ccccc23)cc1 ; c1ccc(-c2ccc3cc(N(c4ccc(-c5ccc6c(c5)sc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4ccc5c(c4)sc4ccccc45)cc3)c3oc4ccccc4c23)cc1 ; c1ccc(-c2ccc(N(c3ccc(-c4ccc5c(c4)oc4ccccc45)cc3)c3ccc(-c4ccc5c(c4)sc4ccccc45)cc3)c3oc4ccccc4c23)cc1]

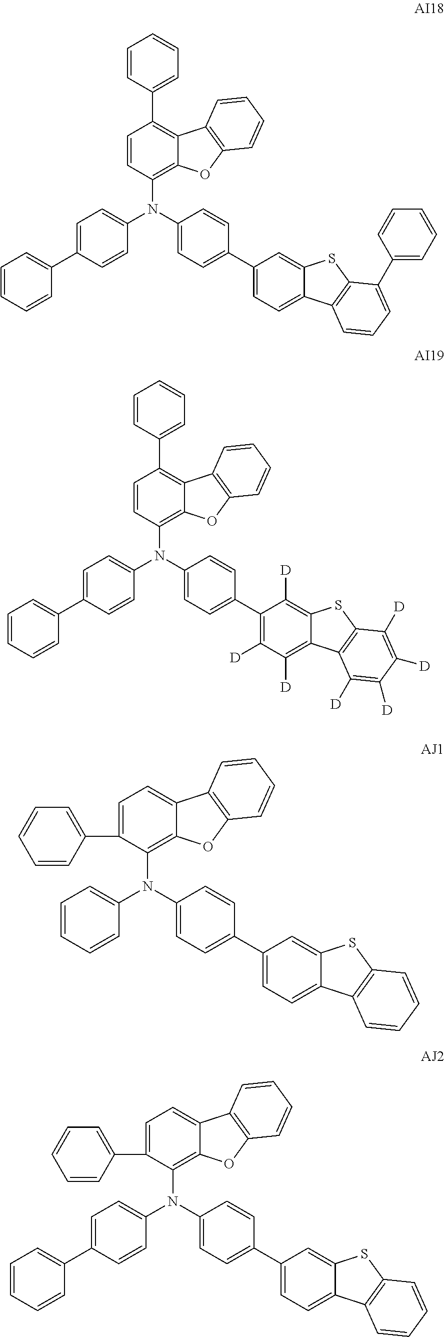 [SMILES: c1ccc(-c2ccc(N(c3ccc(-c4ccc5c(c4)sc4c(-c6ccccc6)cccc45)cc3)c3ccc(-c4ccccc4)c4c3oc3ccccc34)cc2)cc1 ; [2H]c1c([2H])c([2H])c2c(sc3c([2H])c(-c4ccc(N(c5ccc(-c6ccccc6)cc5)c5ccc(-c6ccccc6)c6c5oc5ccccc56)cc4)c([2H])c([2H])c32)c1[2H] ; c1ccc(-c2ccc3c(oc4ccccc43)c2N(c2ccccc2)c2ccc(-c3ccc4c(c3)sc3ccccc34)cc2)cc1 ; c1ccc(-c2ccc(N(c3ccc(-c4ccc5c(c4)sc4ccccc45)cc3)c3c(-c4ccccc4)ccc4c3oc3ccccc34)cc2)cc1]

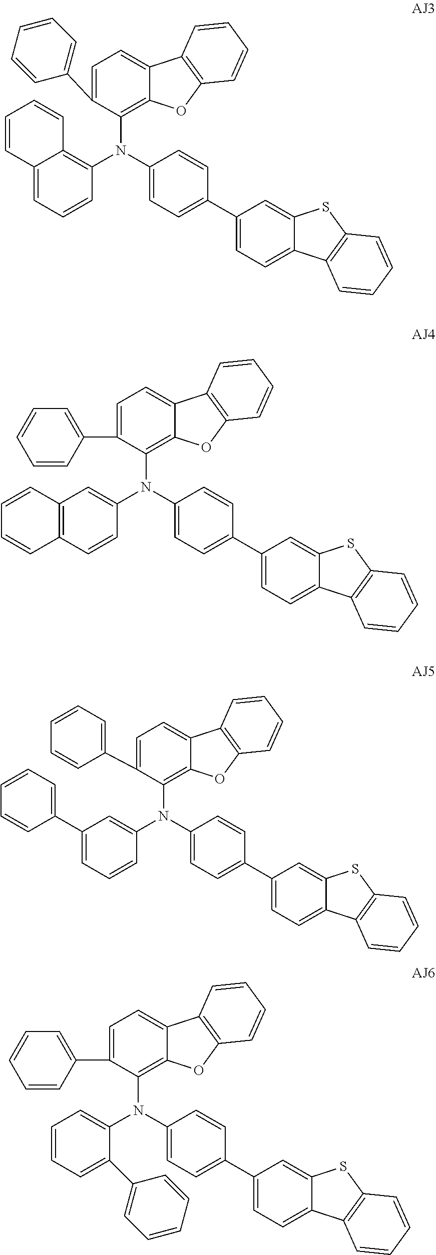 [SMILES: c1ccc(-c2ccc3c(oc4ccccc43)c2N(c2ccc(-c3ccc4c(c3)sc3ccccc34)cc2)c2cccc3ccccc23)cc1 ; c1ccc(-c2ccc3c(oc4ccccc43)c2N(c2ccc(-c3ccc4c(c3)sc3ccccc34)cc2)c2ccc3ccccc3c2)cc1 ; c1ccc(-c2cccc(N(c3ccc(-c4ccc5c(c4)sc4ccccc45)cc3)c3c(-c4ccccc4)ccc4c3oc3ccccc34)c2)cc1 ; c1ccc(-c2ccccc2N(c2ccc(-c3ccc4c(c3)sc3ccccc34)cc2)c2c(-c3ccccc3)ccc3c2oc2ccccc23)cc1]

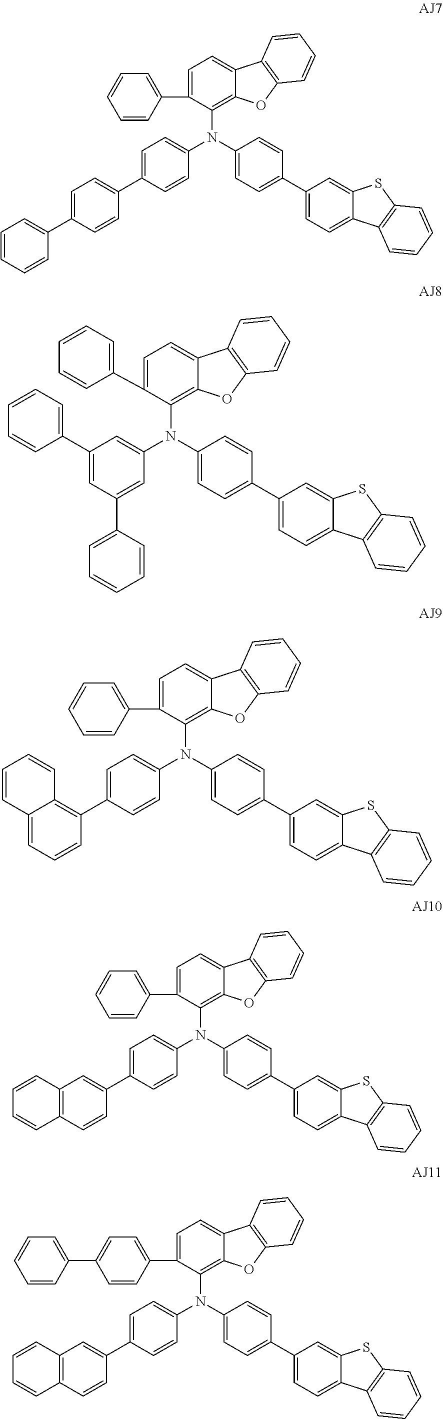 [SMILES: c1ccc(-c2ccc(-c3ccc(N(c4ccc(-c5ccc6c(c5)sc5ccccc56)cc4)c4c(-c5ccccc5)ccc5c4oc4ccccc45)cc3)cc2)cc1 ; c1ccc(-c2cc(-c3ccccc3)cc(N(c3ccc(-c4ccc5c(c4)sc4ccccc45)cc3)c3c(-c4ccccc4)ccc4c3oc3ccccc34)c2)cc1 ; c1ccc(-c2ccc3c(oc4ccccc43)c2N(c2ccc(-c3ccc4c(c3)sc3ccccc34)cc2)c2ccc(-c3cccc4ccccc34)cc2)cc1 ; c1ccc(-c2ccc3c(oc4ccccc43)c2N(c2ccc(-c3ccc4ccccc4c3)cc2)c2ccc(-c3ccc4c(c3)sc3ccccc34)cc2)cc1 ; c1ccc(-c2ccc(-c3ccc4c(oc5ccccc54)c3N(c3ccc(-c4ccc5ccccc5c4)cc3)c3ccc(-c4ccc5c(c4)sc4ccccc45)cc3)cc2)cc1]

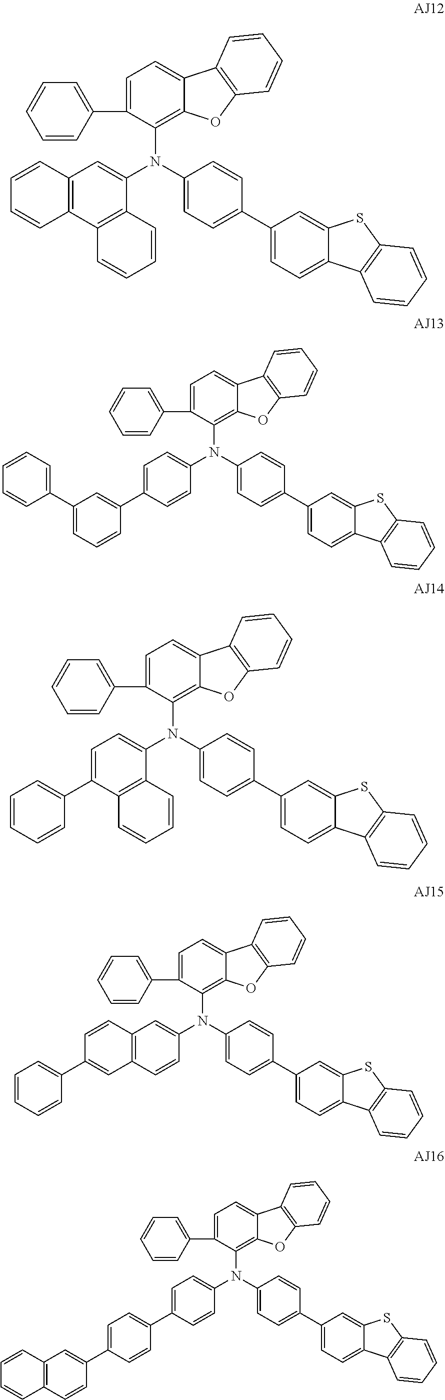 [SMILES: c1ccc(-c2ccc3c(oc4ccccc43)c2N(c2ccc(-c3ccc4c(c3)sc3ccccc34)cc2)c2cc3ccccc3c3ccccc23)cc1 ; c1ccc(-c2cccc(-c3ccc(N(c4ccc(-c5ccc6c(c5)sc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3ccc4c(c3)sc3ccccc34)cc2)c2ccc(-c3ccccc3)c3ccccc23)cc1 ; c1ccc(-c2ccc3cc(N(c4ccc(-c5ccc6c(c5)sc5ccccc56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3ccc4c(c3)sc3ccccc34)cc2)cc1]

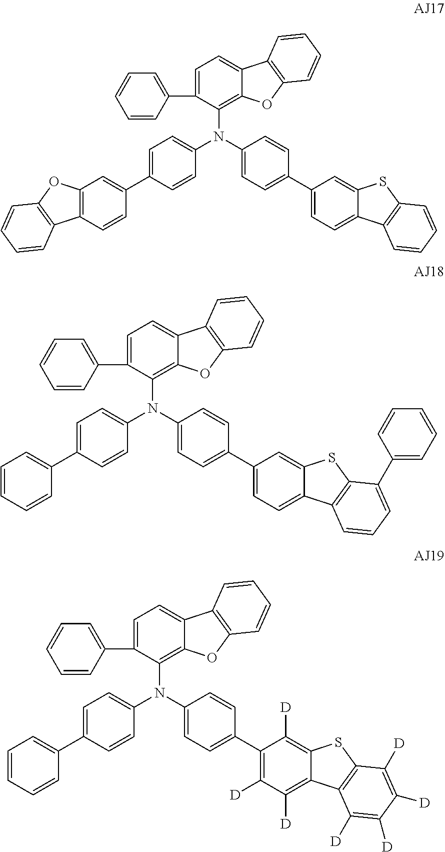 [SMILES: c1ccc(-c2ccc3c(oc4ccccc43)c2N(c2ccc(-c3ccc4c(c3)oc3ccccc34)cc2)c2ccc(-c3ccc4c(c3)sc3ccccc34)cc2)cc1 ; c1ccc(-c2ccc(N(c3ccc(-c4ccc5c(c4)sc4c(-c6ccccc6)cccc45)cc3)c3c(-c4ccccc4)ccc4c3oc3ccccc34)cc2)cc1 ; [2H]c1c([2H])c([2H])c2c(sc3c([2H])c(-c4ccc(N(c5ccc(-c6ccccc6)cc5)c5c(-c6ccccc6)ccc6c5oc5ccccc56)cc4)c([2H])c([2H])c32)c1[2H]]

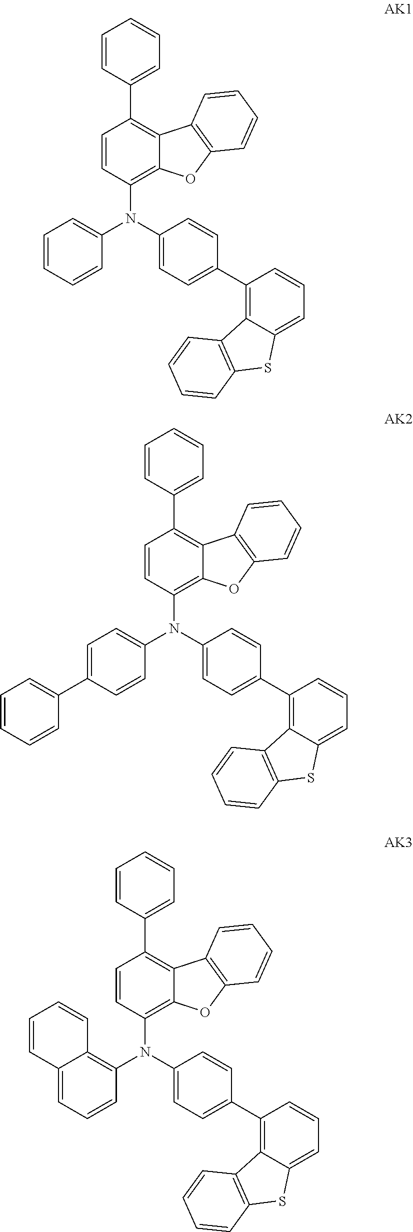

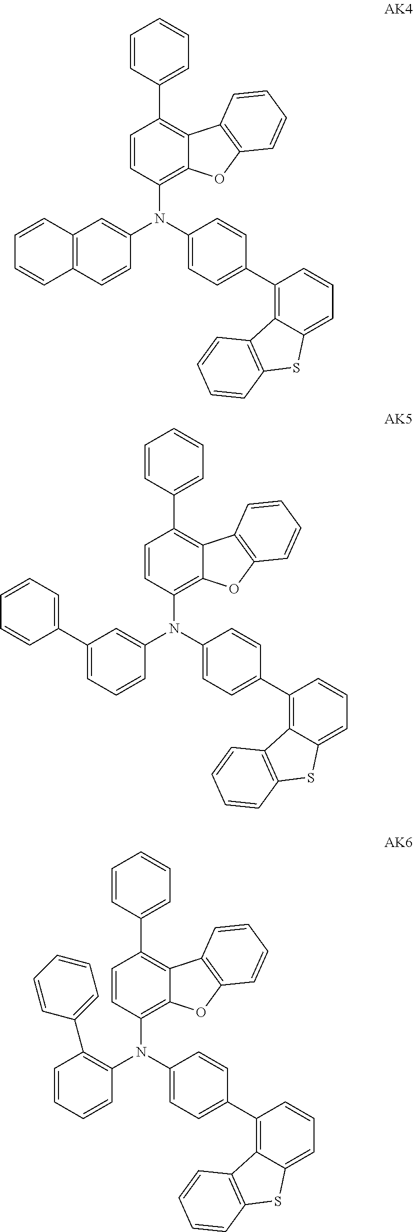 [SMILES: c1ccc(-c2ccc(N(c3ccc(-c4cccc5sc6ccccc6c45)cc3)c3ccc4ccccc4c3)c3oc4ccccc4c23)cc1 ; c1ccc(-c2cccc(N(c3ccc(-c4cccc5sc6ccccc6c45)cc3)c3ccc(-c4ccccc4)c4c3oc3ccccc34)c2)cc1 ; c1ccc(-c2ccccc2N(c2ccc(-c3cccc4sc5ccccc5c34)cc2)c2ccc(-c3ccccc3)c3c2oc2ccccc23)cc1]

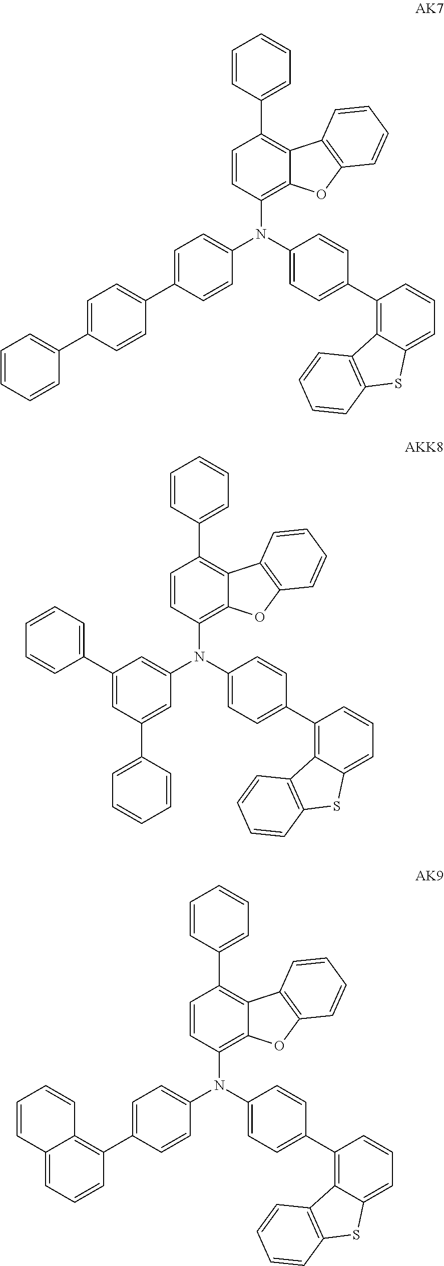 [SMILES: c1ccc(-c2ccc(-c3ccc(N(c4ccc(-c5cccc6sc7ccccc7c56)cc4)c4ccc(-c5ccccc5)c5c4oc4ccccc45)cc3)cc2)cc1 ; c1ccc(-c2cc(-c3ccccc3)cc(N(c3ccc(-c4cccc5sc6ccccc6c45)cc3)c3ccc(-c4ccccc4)c4c3oc3ccccc34)c2)cc1 ; c1ccc(-c2ccc(N(c3ccc(-c4cccc5ccccc45)cc3)c3ccc(-c4cccc5sc6ccccc6c45)cc3)c3oc4ccccc4c23)cc1]

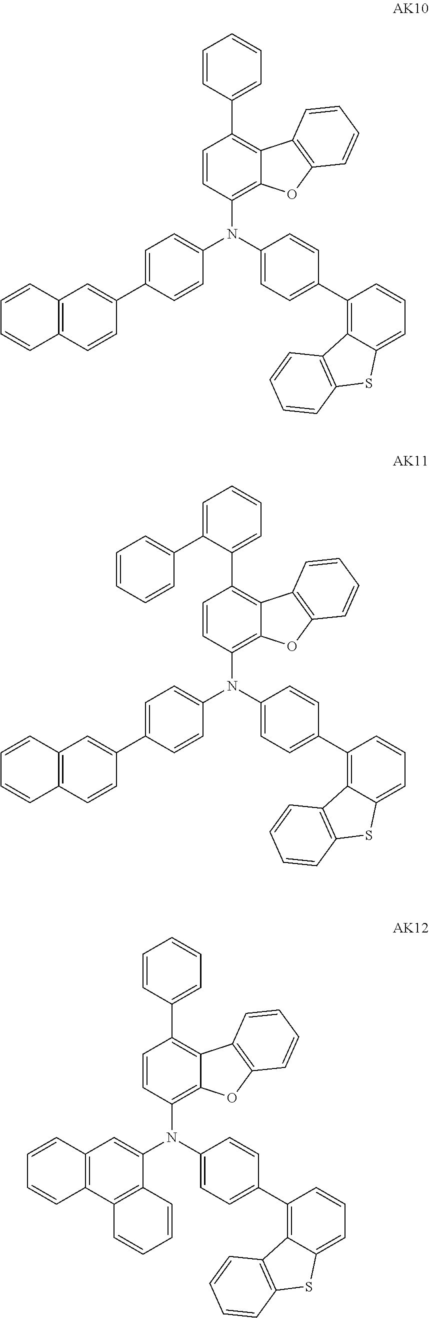 [SMILES: c1ccc(-c2ccc(N(c3ccc(-c4ccc5ccccc5c4)cc3)c3ccc(-c4cccc5sc6ccccc6c45)cc3)c3oc4ccccc4c23)cc1 ; c1ccc(-c2ccccc2-c2ccc(N(c3ccc(-c4ccc5ccccc5c4)cc3)c3ccc(-c4cccc5sc6ccccc6c45)cc3)c3oc4ccccc4c23)cc1 ; c1ccc(-c2ccc(N(c3ccc(-c4cccc5sc6ccccc6c45)cc3)c3cc4ccccc4c4ccccc34)c3oc4ccccc4c23)cc1]

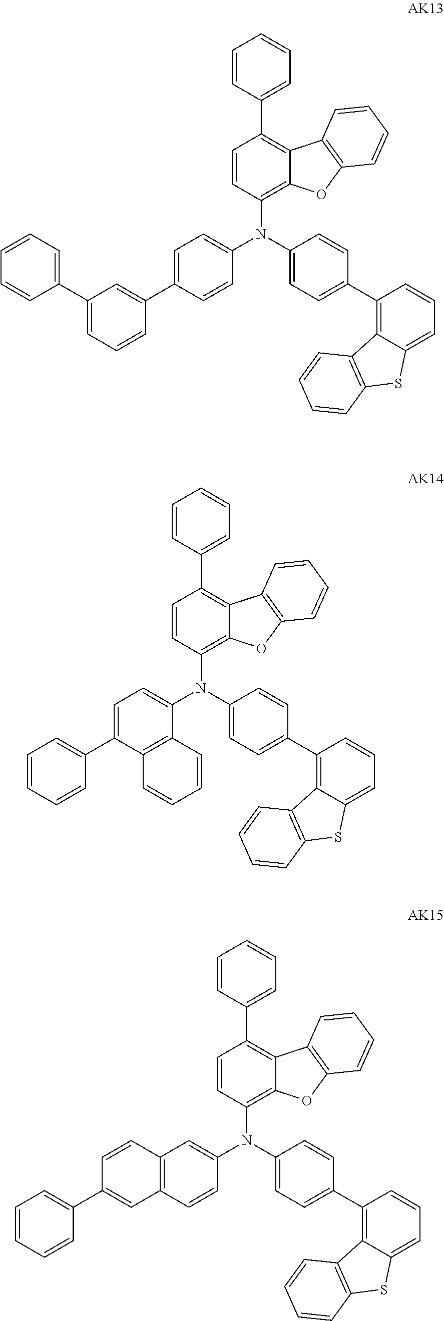 [SMILES: c1ccc(-c2cccc(-c3ccc(N(c4ccc(-c5cccc6sc7ccccc7c56)cc4)c4ccc(-c5ccccc5)c5c4oc4ccccc45)cc3)c2)cc1 ; c1ccc(-c2ccc(N(c3ccc(-c4cccc5sc6ccccc6c45)cc3)c3ccc(-c4ccccc4)c4c3oc3ccccc34)c3ccccc23)cc1 ; c1ccc(-c2ccc3cc(N(c4ccc(-c5cccc6sc7ccccc7c56)cc4)c4ccc(-c5ccccc5)c5c4oc4ccccc45)ccc3c2)cc1]

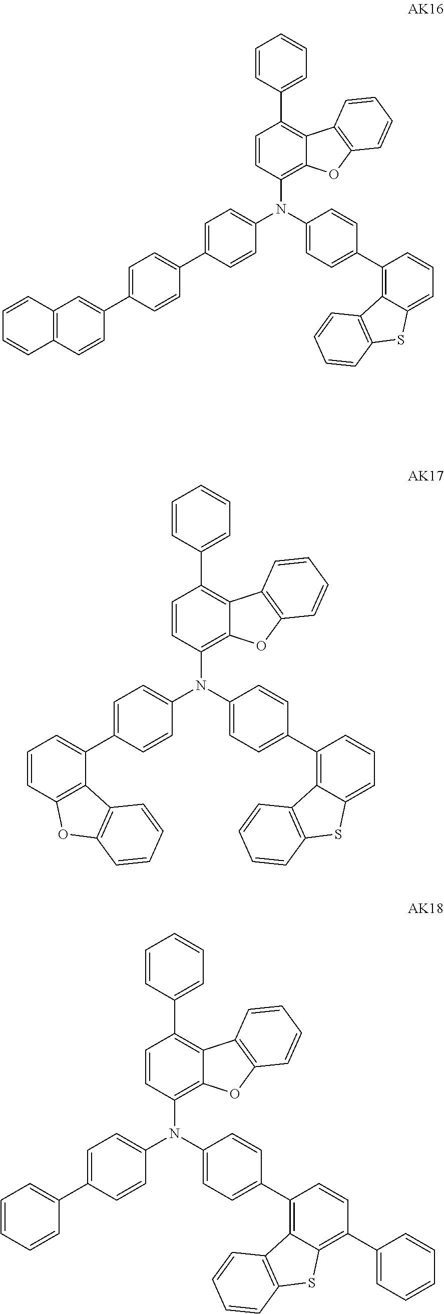 [SMILES: c1ccc(-c2ccc(N(c3ccc(-c4ccc(-c5ccc6ccccc6c5)cc4)cc3)c3ccc(-c4cccc5sc6ccccc6c45)cc3)c3oc4ccccc4c23)cc1 ; c1ccc(-c2ccc(N(c3ccc(-c4cccc5oc6ccccc6c45)cc3)c3ccc(-c4cccc5sc6ccccc6c45)cc3)c3oc4ccccc4c23)cc1 ; c1ccc(-c2ccc(N(c3ccc(-c4ccc(-c5ccccc5)c5sc6ccccc6c45)cc3)c3ccc(-c4ccccc4)c4c3oc3ccccc34)cc2)cc1]

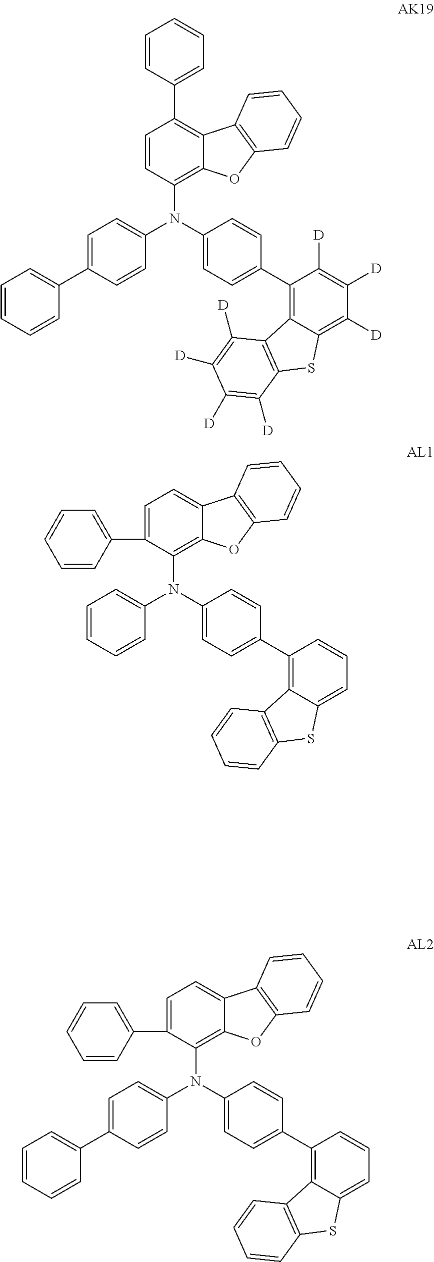

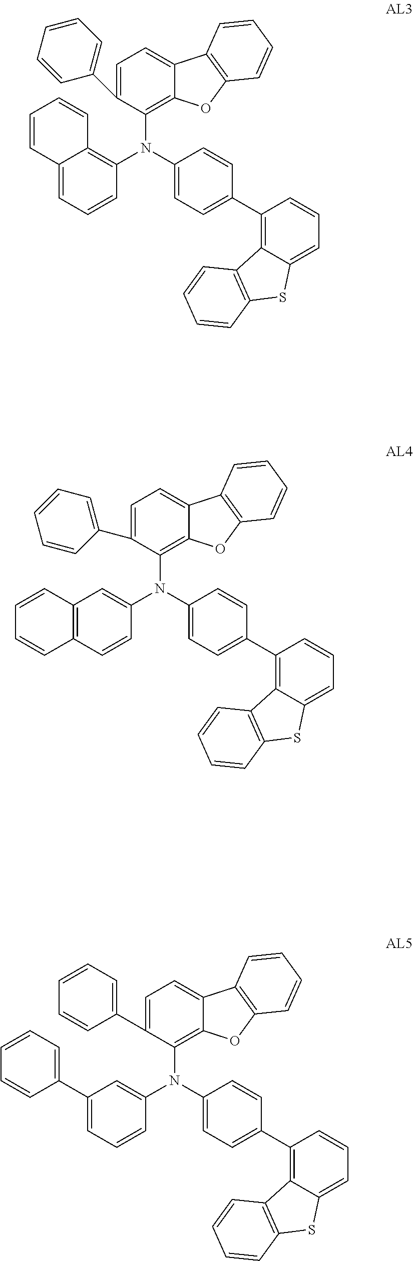 [SMILES: c1ccc(-c2ccc3c(oc4ccccc43)c2N(c2ccc(-c3cccc4sc5ccccc5c34)cc2)c2cccc3ccccc23)cc1 ; c1ccc(-c2ccc3c(oc4ccccc43)c2N(c2ccc(-c3cccc4sc5ccccc5c34)cc2)c2ccc3ccccc3c2)cc1 ; c1ccc(-c2cccc(N(c3ccc(-c4cccc5sc6ccccc6c45)cc3)c3c(-c4ccccc4)ccc4c3oc3ccccc34)c2)cc1]

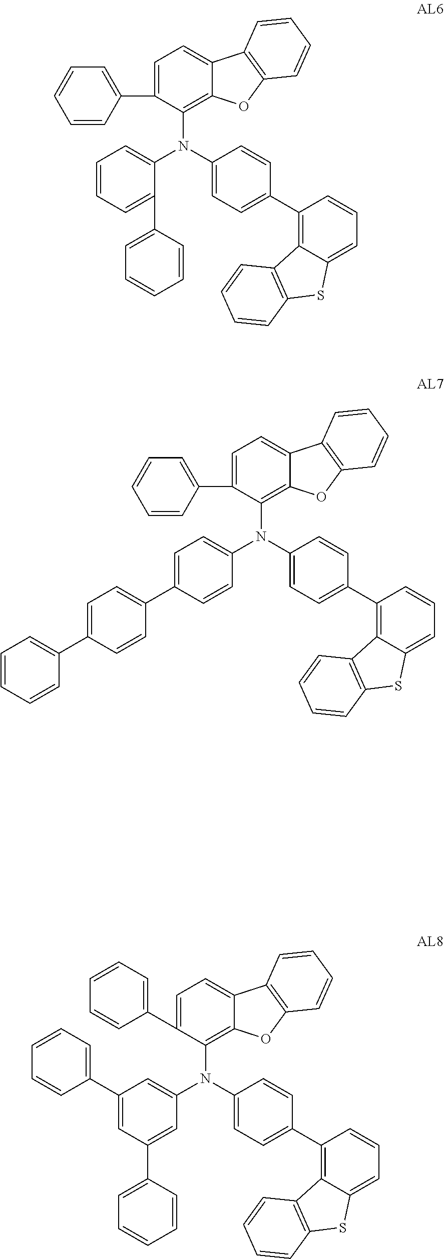 [SMILES: c1ccc(-c2ccccc2N(c2ccc(-c3cccc4sc5ccccc5c34)cc2)c2c(-c3ccccc3)ccc3c2oc2ccccc23)cc1 ; c1ccc(-c2ccc(-c3ccc(N(c4ccc(-c5cccc6sc7ccccc7c56)cc4)c4c(-c5ccccc5)ccc5c4oc4ccccc45)cc3)cc2)cc1 ; c1ccc(-c2cc(-c3ccccc3)cc(N(c3ccc(-c4cccc5sc6ccccc6c45)cc3)c3c(-c4ccccc4)ccc4c3oc3ccccc34)c2)cc1]

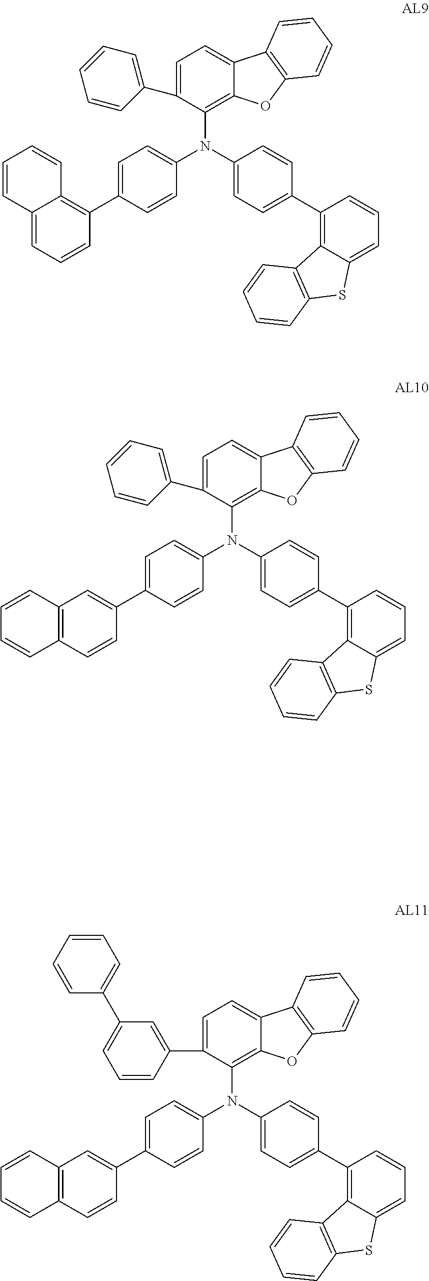 [SMILES: c1ccc(-c2ccc3c(oc4ccccc43)c2N(c2ccc(-c3cccc4ccccc34)cc2)c2ccc(-c3cccc4sc5ccccc5c34)cc2)cc1 ; c1ccc(-c2ccc3c(oc4ccccc43)c2N(c2ccc(-c3ccc4ccccc4c3)cc2)c2ccc(-c3cccc4sc5ccccc5c34)cc2)cc1 ; c1ccc(-c2cccc(-c3ccc4c(oc5ccccc54)c3N(c3ccc(-c4ccc5ccccc5c4)cc3)c3ccc(-c4cccc5sc6ccccc6c45)cc3)c2)cc1]

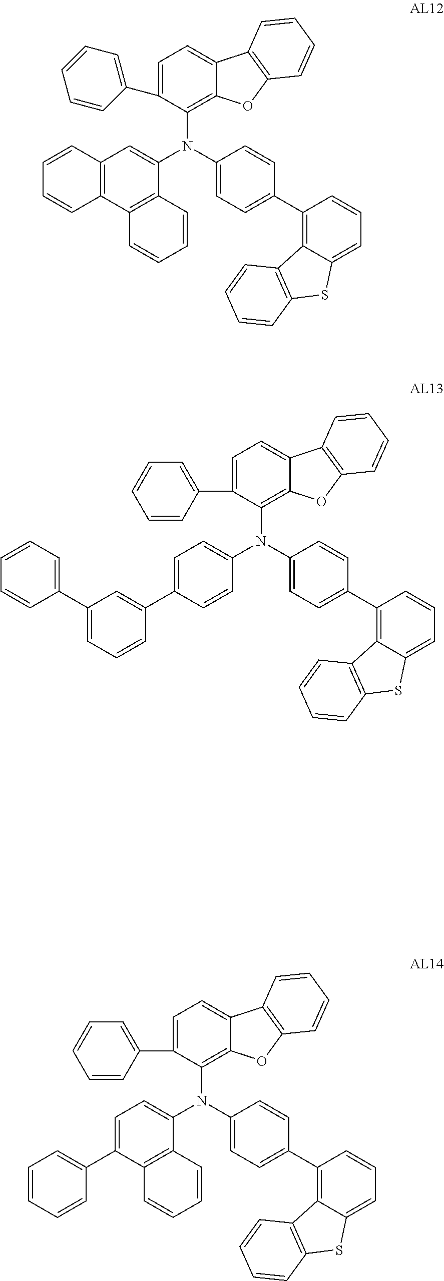 [SMILES: c1ccc(-c2ccc3c(oc4ccccc43)c2N(c2ccc(-c3cccc4sc5ccccc5c34)cc2)c2cc3ccccc3c3ccccc23)cc1 ; c1ccc(-c2cccc(-c3ccc(N(c4ccc(-c5cccc6sc7ccccc7c56)cc4)c4c(-c5ccccc5)ccc5c4oc4ccccc45)cc3)c2)cc1 ; c1ccc(-c2ccc3c(oc4ccccc43)c2N(c2ccc(-c3cccc4sc5ccccc5c34)cc2)c2ccc(-c3ccccc3)c3ccccc23)cc1]

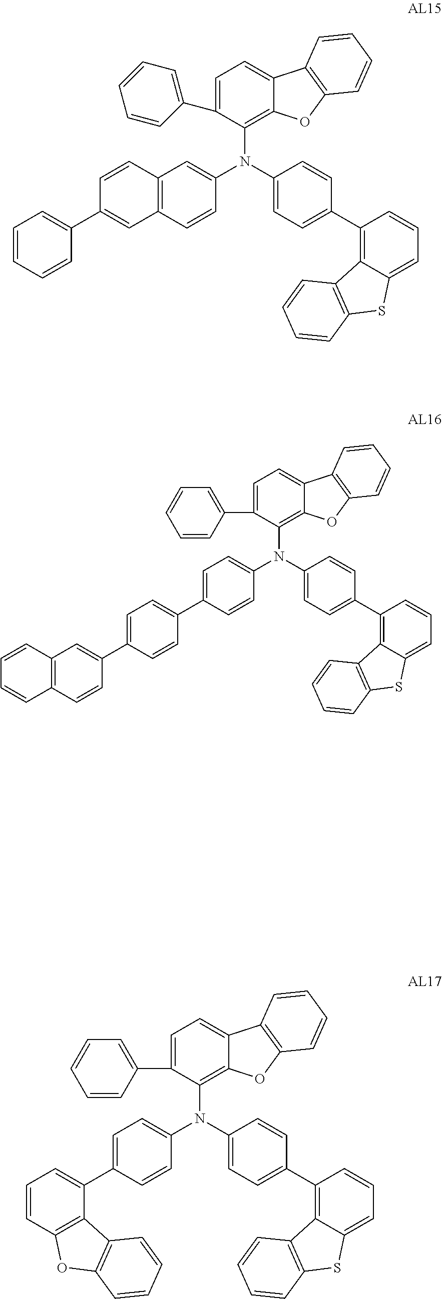 [SMILES: c1ccc(-c2ccc3cc(N(c4ccc(-c5cccc6sc7ccccc7c56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4sc5ccccc5c34)cc2)cc1 ; c1ccc(-c2ccc3c(oc4ccccc43)c2N(c2ccc(-c3cccc4oc5ccccc5c34)cc2)c2ccc(-c3cccc4sc5ccccc5c34)cc2)cc1]

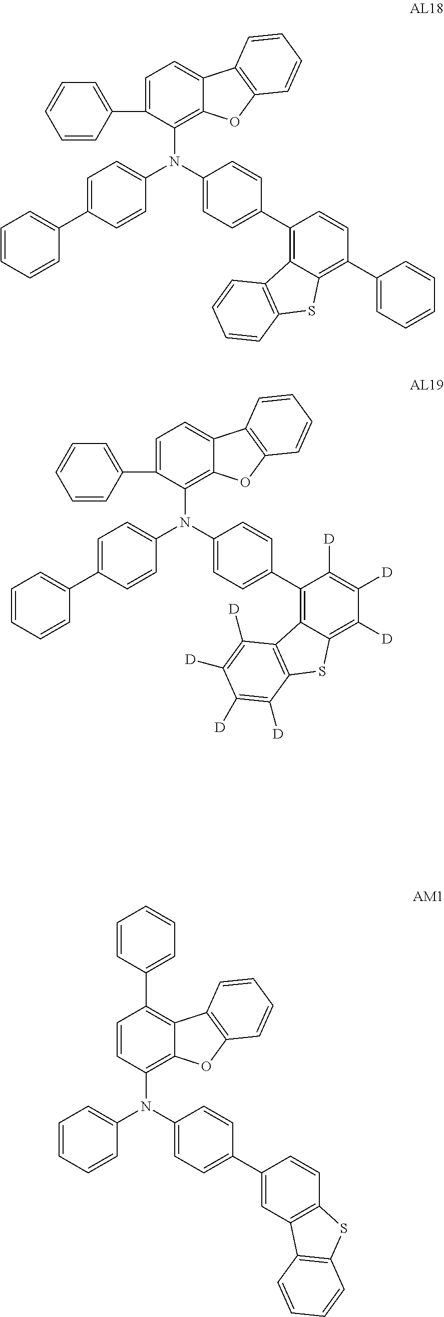 [SMILES: c1ccc(-c2ccc(N(c3ccc(-c4ccc(-c5ccccc5)c5sc6ccccc6c45)cc3)c3c(-c4ccccc4)ccc4c3oc3ccccc34)cc2)cc1 ; [2H]c1c([2H])c([2H])c2c(sc3c([2H])c([2H])c([2H])c(-c4ccc(N(c5ccc(-c6ccccc6)cc5)c5c(-c6ccccc6)ccc6c5oc5ccccc56)cc4)c32)c1[2H] ; c1ccc(-c2ccc(N(c3ccccc3)c3ccc(-c4ccc5sc6ccccc6c5c4)cc3)c3oc4ccccc4c23)cc1]

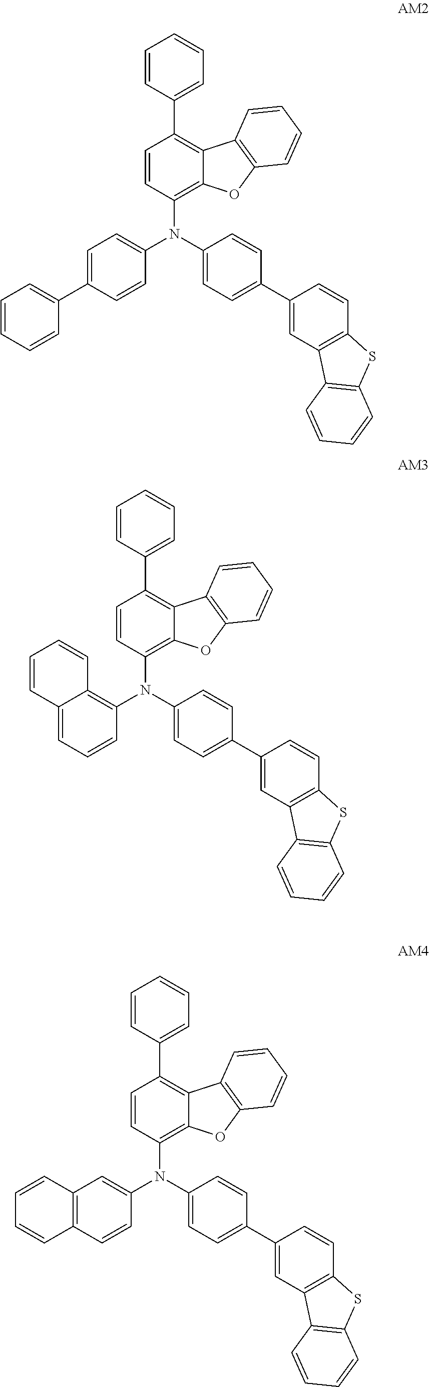 [SMILES: c1ccc(-c2ccc(N(c3ccc(-c4ccc5sc6ccccc6c5c4)cc3)c3ccc(-c4ccccc4)c4c3oc3ccccc34)cc2)cc1 ; c1ccc(-c2ccc(N(c3ccc(-c4ccc5sc6ccccc6c5c4)cc3)c3cccc4ccccc34)c3oc4ccccc4c23)cc1 ; c1ccc(-c2ccc(N(c3ccc(-c4ccc5sc6ccccc6c5c4)cc3)c3ccc4ccccc4c3)c3oc4ccccc4c23)cc1]

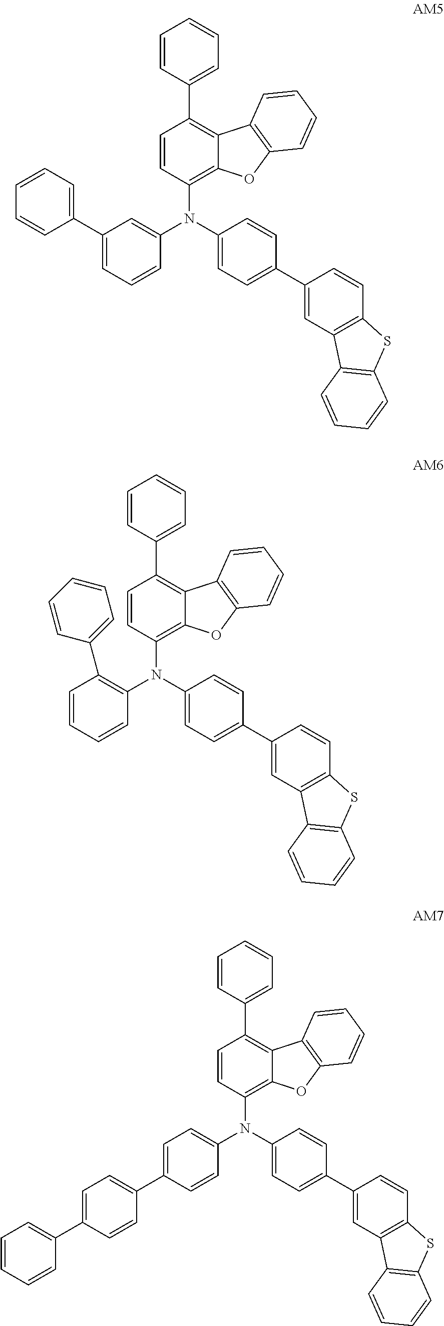 [SMILES: c1ccc(-c2cccc(N(c3ccc(-c4ccc5sc6ccccc6c5c4)cc3)c3ccc(-c4ccccc4)c4c3oc3ccccc34)c2)cc1 ; c1ccc(-c2ccccc2N(c2ccc(-c3ccc4sc5ccccc5c4c3)cc2)c2ccc(-c3ccccc3)c3c2oc2ccccc23)cc1 ; c1ccc(-c2ccc(-c3ccc(N(c4ccc(-c5ccc6sc7ccccc7c6c5)cc4)c4ccc(-c5ccccc5)c5c4oc4ccccc45)cc3)cc2)cc1]

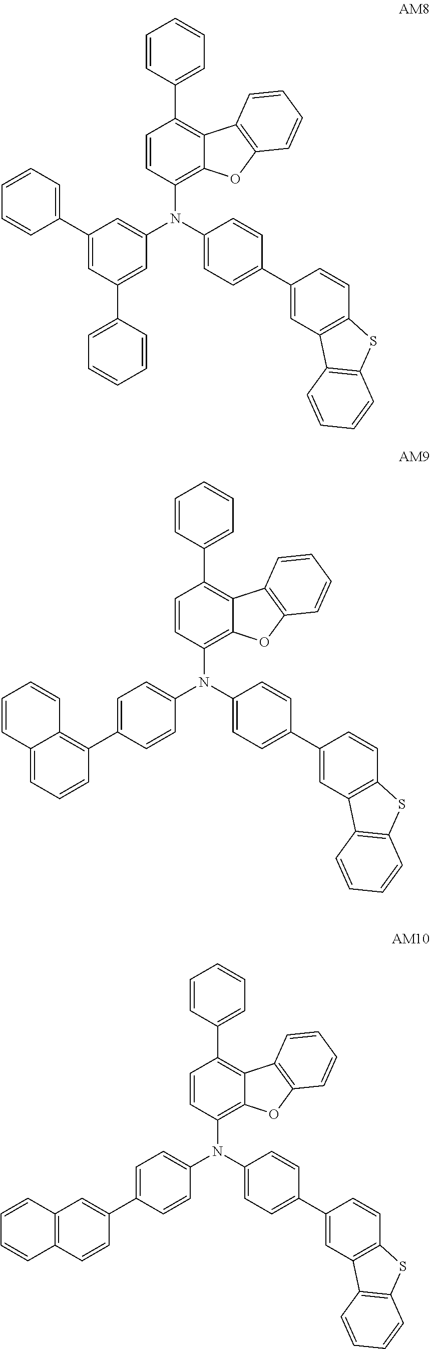 [SMILES: c1ccc(-c2cc(-c3ccccc3)cc(N(c3ccc(-c4ccc5sc6ccccc6c5c4)cc3)c3ccc(-c4ccccc4)c4c3oc3ccccc34)c2)cc1 ; c1ccc(-c2ccc(N(c3ccc(-c4ccc5sc6ccccc6c5c4)cc3)c3ccc(-c4cccc5ccccc45)cc3)c3oc4ccccc4c23)cc1 ; c1ccc(-c2ccc(N(c3ccc(-c4ccc5ccccc5c4)cc3)c3ccc(-c4ccc5sc6ccccc6c5c4)cc3)c3oc4ccccc4c23)cc1]

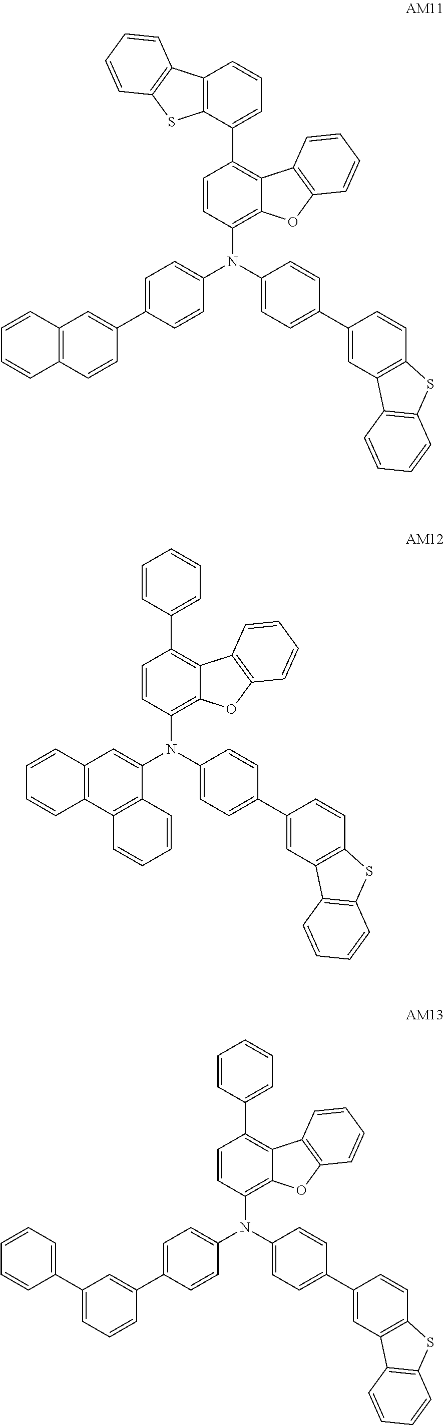 [SMILES: c1ccc2cc(-c3ccc(N(c4ccc(-c5ccc6sc7ccccc7c6c5)cc4)c4ccc(-c5cccc6c5sc5ccccc56)c5c4oc4ccccc45)cc3)ccc2c1 ; c1ccc(-c2ccc(N(c3ccc(-c4ccc5sc6ccccc6c5c4)cc3)c3cc4ccccc4c4ccccc34)c3oc4ccccc4c23)cc1 ; c1ccc(-c2cccc(-c3ccc(N(c4ccc(-c5ccc6sc7ccccc7c6c5)cc4)c4ccc(-c5ccccc5)c5c4oc4ccccc45)cc3)c2)cc1]

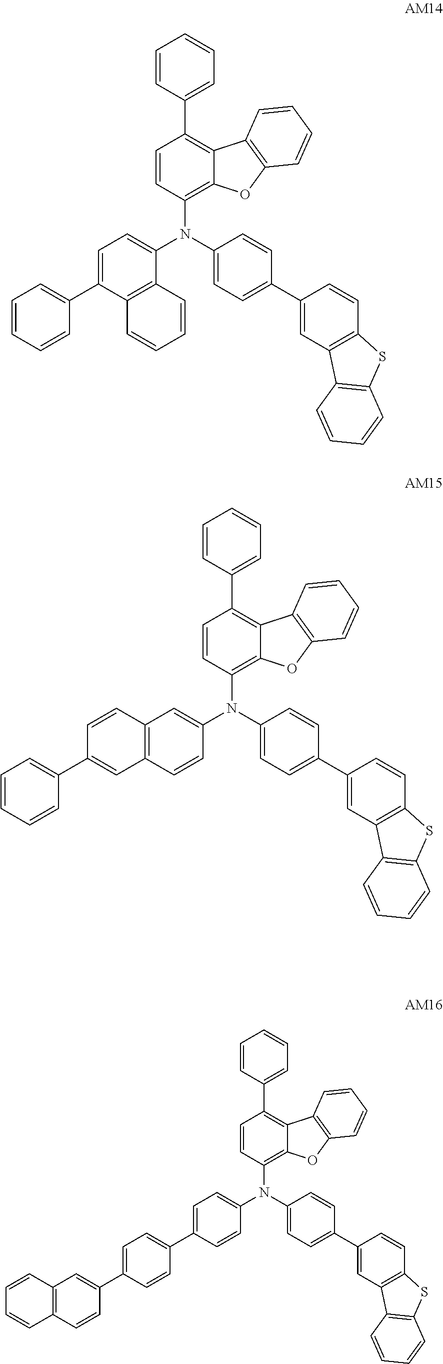 [SMILES: c1ccc(-c2ccc(N(c3ccc(-c4ccc5sc6ccccc6c5c4)cc3)c3ccc(-c4ccccc4)c4c3oc3ccccc34)c3ccccc23)cc1 ; c1ccc(-c2ccc3cc(N(c4ccc(-c5ccc6sc7ccccc7c6c5)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4ccc5sc6ccccc6c5c4)cc3)c3oc4ccccc4c23)cc1]

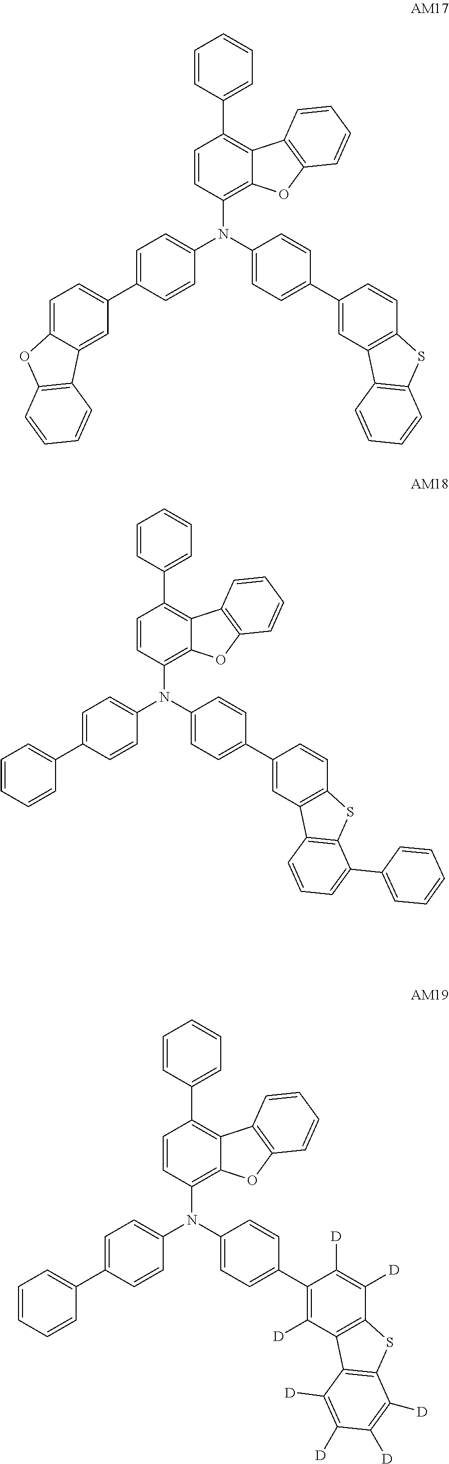 [SMILES: c1ccc(-c2ccc(N(c3ccc(-c4ccc5oc6ccccc6c5c4)cc3)c3ccc(-c4ccc5sc6ccccc6c5c4)cc3)c3oc4ccccc4c23)cc1 ; c1ccc(-c2ccc(N(c3ccc(-c4ccc5sc6c(-c7ccccc7)cccc6c5c4)cc3)c3ccc(-c4ccccc4)c4c3oc3ccccc34)cc2)cc1 ; [2H]c1c([2H])c([2H])c2c(sc3c([2H])c([2H])c(-c4ccc(N(c5ccc(-c6ccccc6)cc5)c5ccc(-c6ccccc6)c6c5oc5ccccc56)cc4)c([2H])c32)c1[2H]]

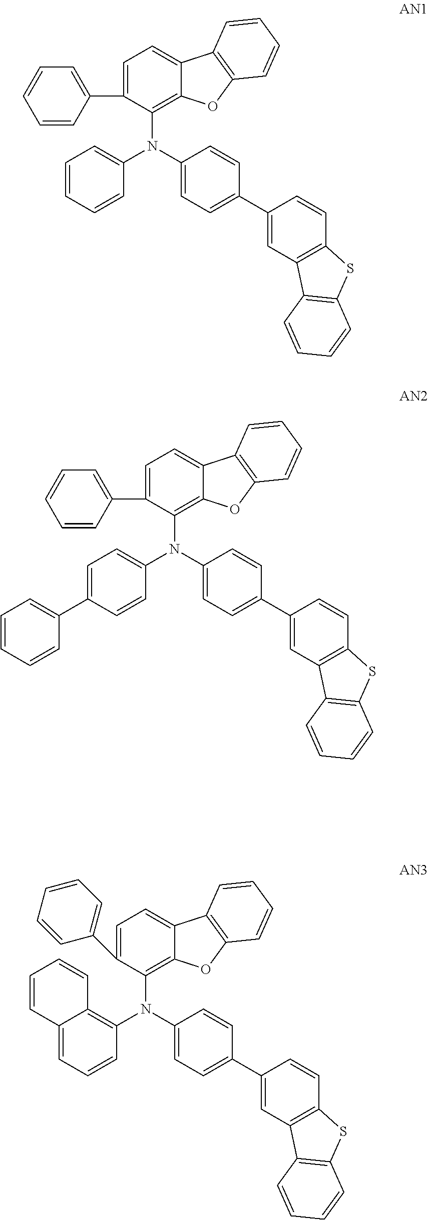 [SMILES: c1ccc(-c2ccc3c(oc4ccccc43)c2N(c2ccccc2)c2ccc(-c3ccc4sc5ccccc5c4c3)cc2)cc1 ; c1ccc(-c2ccc(N(c3ccc(-c4ccc5sc6ccccc6c5c4)cc3)c3c(-c4ccccc4)ccc4c3oc3ccccc34)cc2)cc1 ; c1ccc(-c2ccc3c(oc4ccccc43)c2N(c2ccc(-c3ccc4sc5ccccc5c4c3)cc2)c2cccc3ccccc23)cc1]

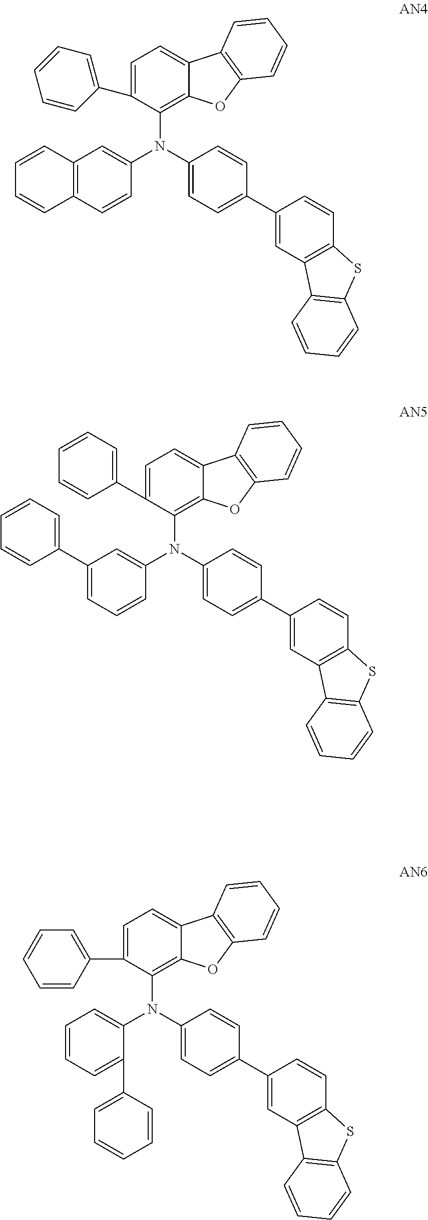 [SMILES: c1ccc(-c2ccc3c(oc4ccccc43)c2N(c2ccc(-c3ccc4sc5ccccc5c4c3)cc2)c2ccc3ccccc3c2)cc1 ; c1ccc(-c2cccc(N(c3ccc(-c4ccc5sc6ccccc6c5c4)cc3)c3c(-c4ccccc4)ccc4c3oc3ccccc34)c2)cc1 ; c1ccc(-c2ccccc2N(c2ccc(-c3ccc4sc5ccccc5c4c3)cc2)c2c(-c3ccccc3)ccc3c2oc2ccccc23)cc1]

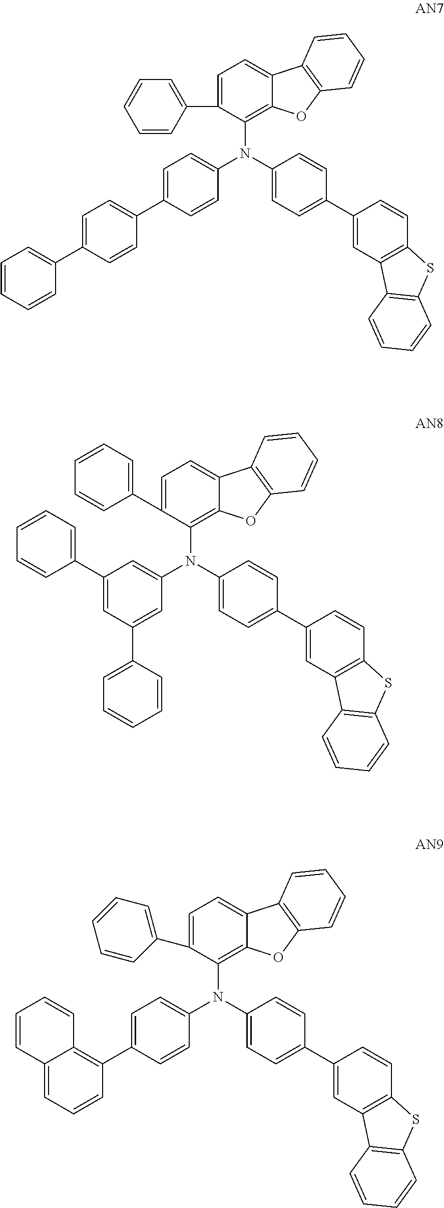 [SMILES: c1ccc(-c2ccc(-c3ccc(N(c4ccc(-c5ccc6sc7ccccc7c6c5)cc4)c4c(-c5ccccc5)ccc5c4oc4ccccc45)cc3)cc2)cc1 ; c1ccc(-c2cc(-c3ccccc3)cc(N(c3ccc(-c4ccc5sc6ccccc6c5c4)cc3)c3c(-c4ccccc4)ccc4c3oc3ccccc34)c2)cc1 ; c1ccc(-c2ccc3c(oc4ccccc43)c2N(c2ccc(-c3ccc4sc5ccccc5c4c3)cc2)c2ccc(-c3cccc4ccccc34)cc2)cc1]

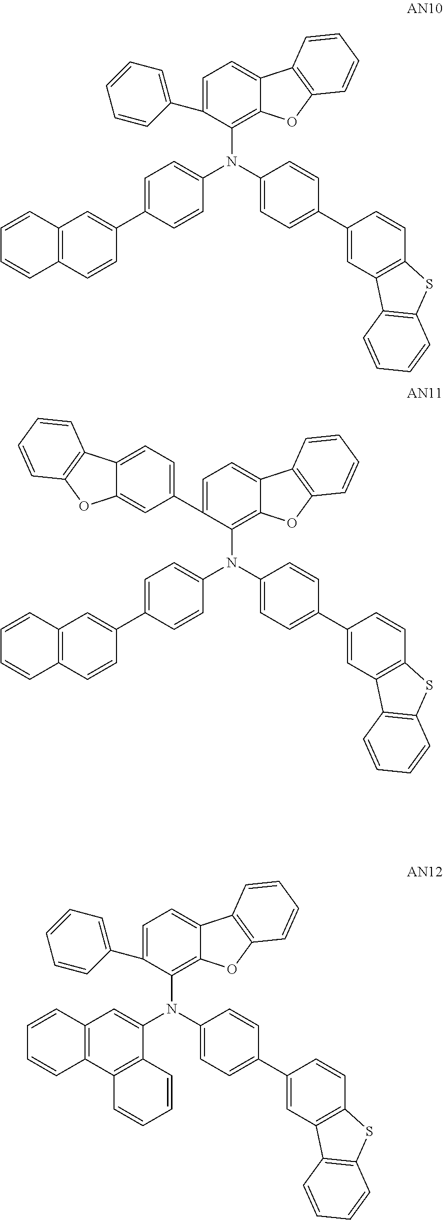 [SMILES: c1ccc(-c2ccc3c(oc4ccccc43)c2N(c2ccc(-c3ccc4ccccc4c3)cc2)c2ccc(-c3ccc4sc5ccccc5c4c3)cc2)cc1 ; c1ccc2cc(-c3ccc(N(c4ccc(-c5ccc6sc7ccccc7c6c5)cc4)c4c(-c5ccc6c(c5)oc5ccccc56)ccc5c4oc4ccccc45)cc3)ccc2c1 ; c1ccc(-c2ccc3c(oc4ccccc43)c2N(c2ccc(-c3ccc4sc5ccccc5c4c3)cc2)c2cc3ccccc3c3ccccc23)cc1]

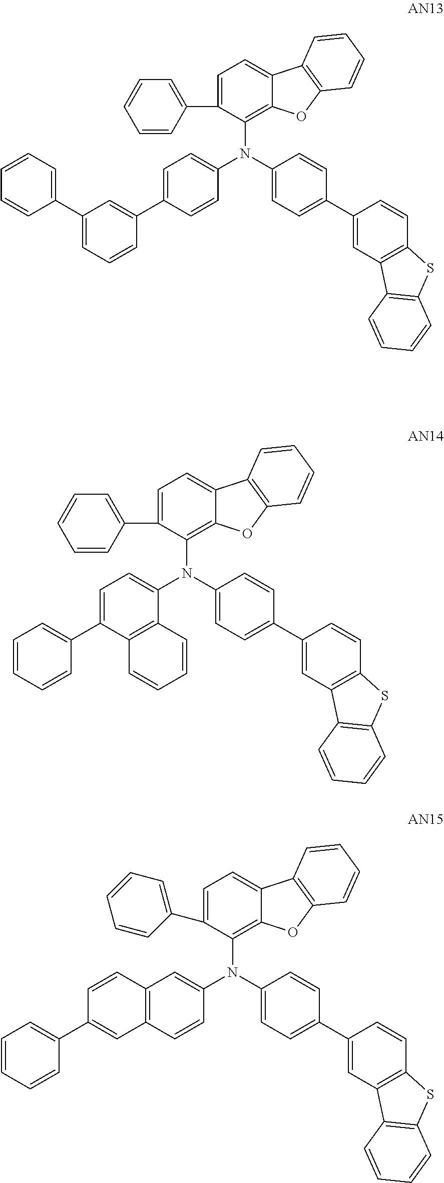 [SMILES: c1ccc(-c2cccc(-c3ccc(N(c4ccc(-c5ccc6sc7ccccc7c6c5)cc4)c4c(-c5ccccc5)ccc5c4oc4ccccc45)cc3)c2)cc1 ; c1ccc(-c2ccc3c(oc4ccccc43)c2N(c2ccc(-c3ccc4sc5ccccc5c4c3)cc2)c2ccc(-c3ccccc3)c3ccccc23)cc1 ; c1ccc(-c2ccc3cc(N(c4ccc(-c5ccc6sc7ccccc7c6c5)cc4)c4c(-c5ccccc5)ccc5c4oc4ccccc45)ccc3c2)cc1]

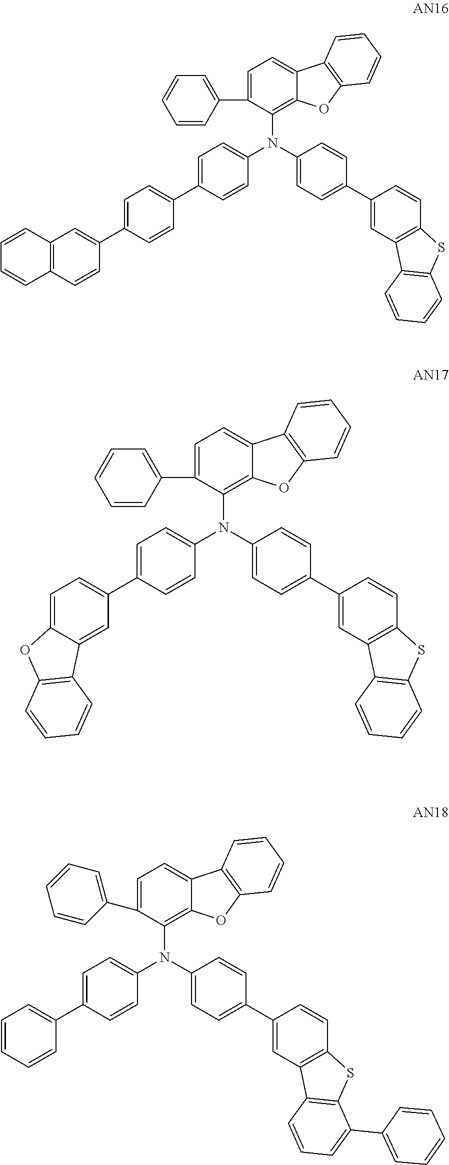 [SMILES: c1ccc(-c2ccc3c(oc4ccccc43)c2N(c2ccc(-c3ccc(-c4ccc5ccccc5c4)cc3)cc2)c2ccc(-c3ccc4sc5ccccc5c4c3)cc2)cc1 ; c1ccc(-c2ccc3c(oc4ccccc43)c2N(c2ccc(-c3ccc4oc5ccccc5c4c3)cc2)c2ccc(-c3ccc4sc5ccccc5c4c3)cc2)cc1 ; c1ccc(-c2ccc(N(c3ccc(-c4ccc5sc6c(-c7ccccc7)cccc6c5c4)cc3)c3c(-c4ccccc4)ccc4c3oc3ccccc34)cc2)cc1]

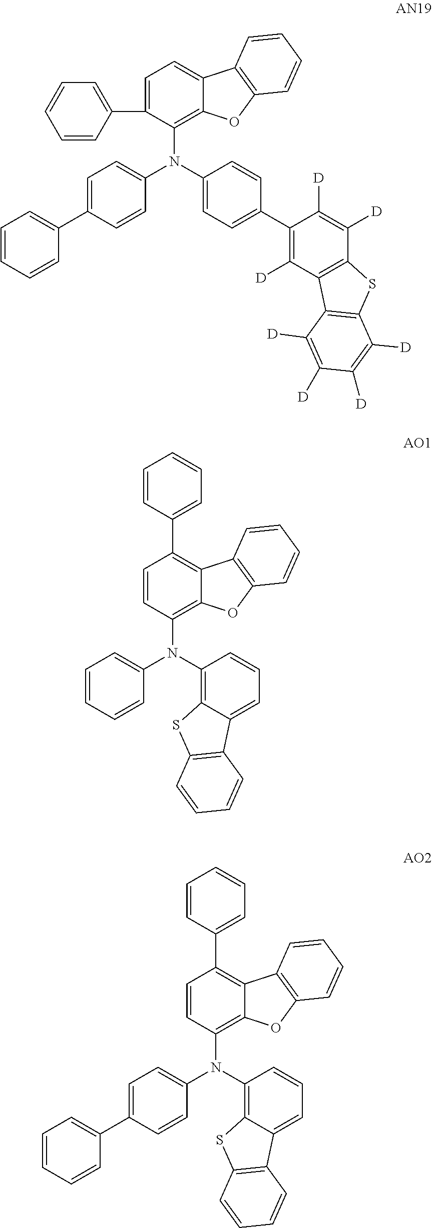 [SMILES: [2H]c1c([2H])c([2H])c2c(sc3c([2H])c([2H])c(-c4ccc(N(c5ccc(-c6ccccc6)cc5)c5c(-c6ccccc6)ccc6c5oc5ccccc56)cc4)c([2H])c32)c1[2H] ; c1ccc(-c2ccc(N(c3ccccc3)c3cccc4c3sc3ccccc34)c3oc4ccccc4c23)cc1 ; c1ccc(-c2ccc(N(c3ccc(-c4ccccc4)c4c3oc3ccccc34)c3cccc4c3sc3ccccc34)cc2)cc1]

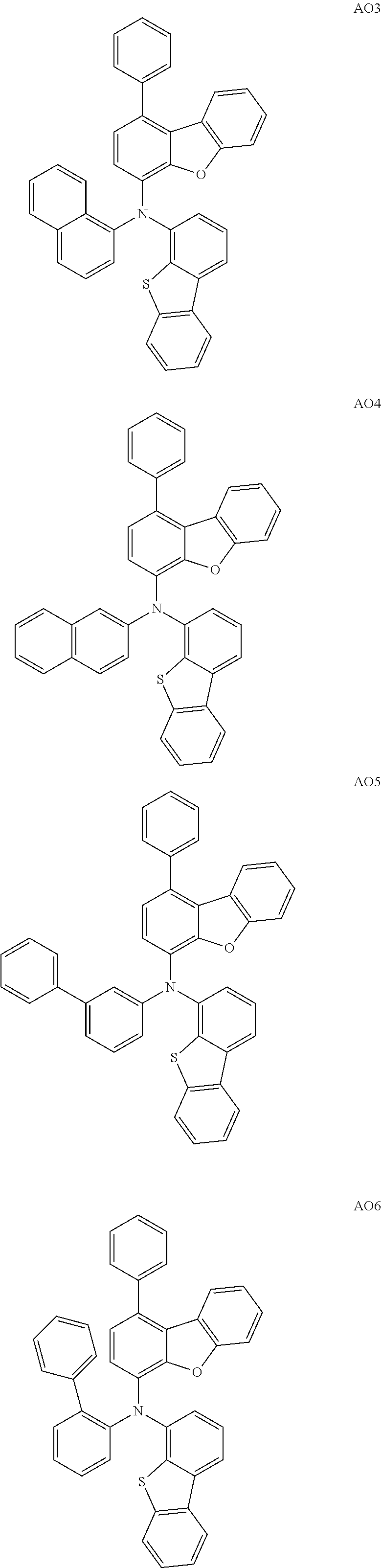

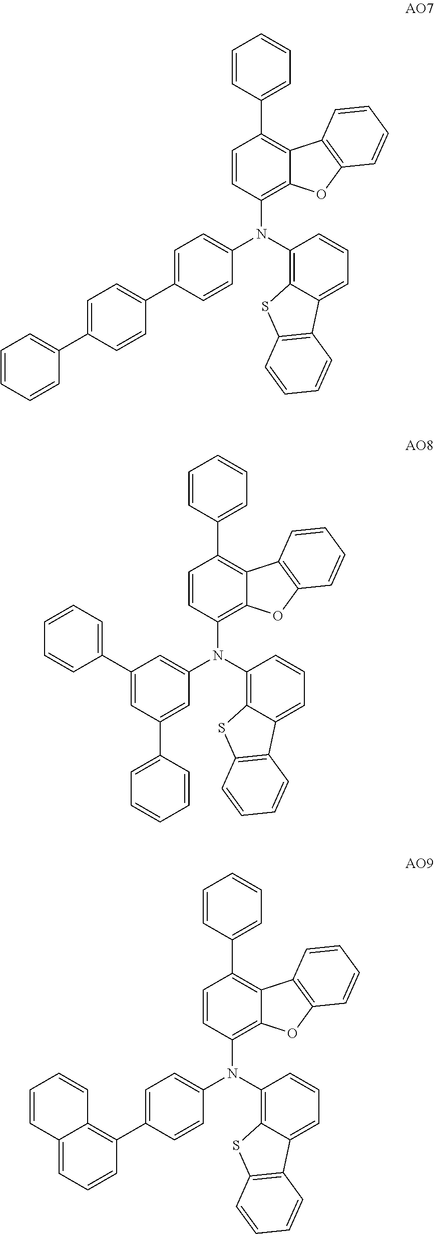 [SMILES: c1ccc(-c2ccc(-c3ccc(N(c4ccc(-c5ccccc5)c5c4oc4ccccc45)c4cccc5c4sc4ccccc45)cc3)cc2)cc1 ; c1ccc(-c2cc(-c3ccccc3)cc(N(c3ccc(-c4ccccc4)c4c3oc3ccccc34)c3cccc4c3sc3ccccc34)c2)cc1 ; c1ccc(-c2ccc(N(c3ccc(-c4cccc5ccccc45)cc3)c3cccc4c3sc3ccccc34)c3oc4ccccc4c23)cc1]

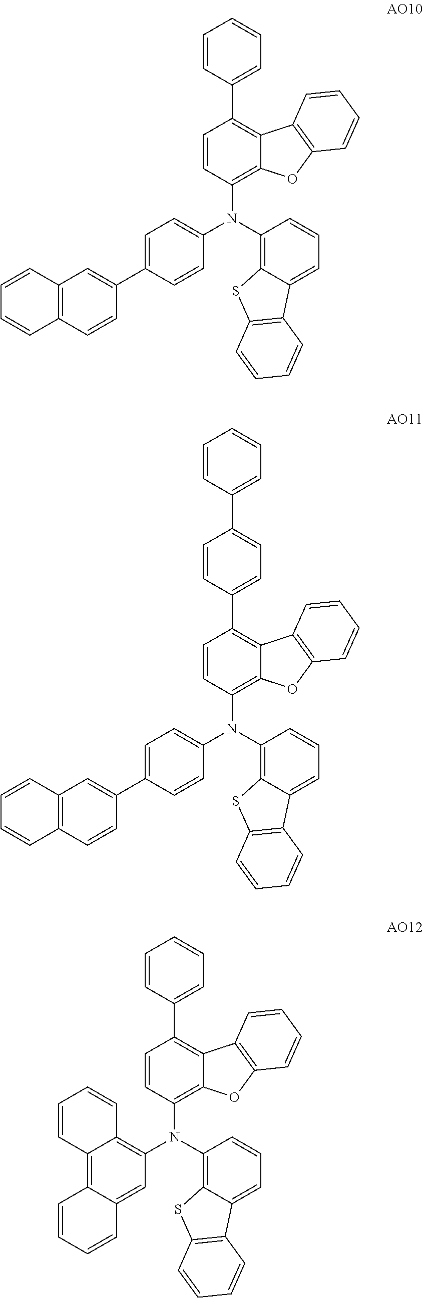 [SMILES: c1ccc(-c2ccc(N(c3ccc(-c4ccc5ccccc5c4)cc3)c3cccc4c3sc3ccccc34)c3oc4ccccc4c23)cc1 ; c1ccc(-c2ccc(-c3ccc(N(c4ccc(-c5ccc6ccccc6c5)cc4)c4cccc5c4sc4ccccc45)c4oc5ccccc5c34)cc2)cc1 ; c1ccc(-c2ccc(N(c3cc4ccccc4c4ccccc34)c3cccc4c3sc3ccccc34)c3oc4ccccc4c23)cc1]

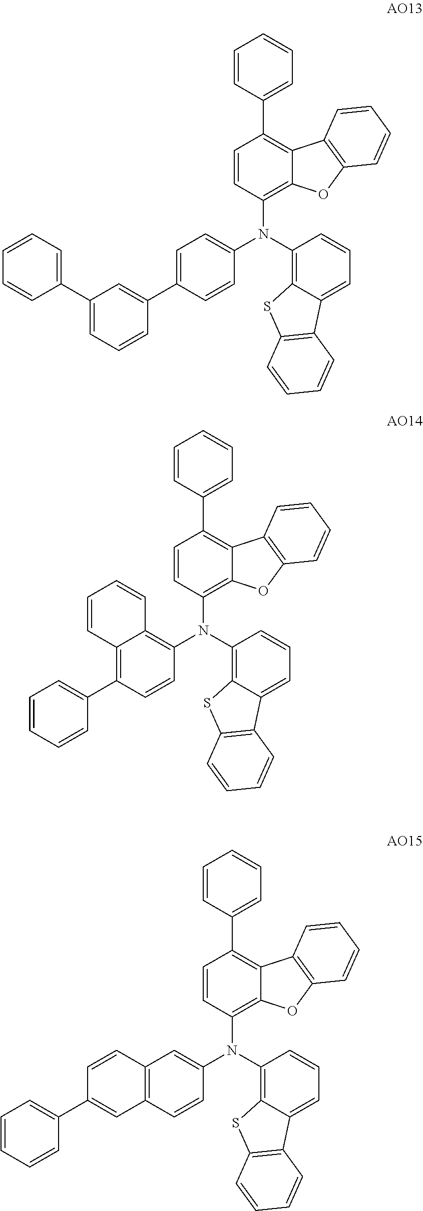 [SMILES: c1ccc(-c2cccc(-c3ccc(N(c4ccc(-c5ccccc5)c5c4oc4ccccc45)c4cccc5c4sc4ccccc45)cc3)c2)cc1 ; c1ccc(-c2ccc(N(c3ccc(-c4ccccc4)c4c3oc3ccccc34)c3cccc4c3sc3ccccc34)c3ccccc23)cc1 ; c1ccc(-c2ccc3cc(N(c4ccc(-c5ccccc5)c5c4oc4ccccc45)c4cccc5c4sc4ccccc45)ccc3c2)cc1]

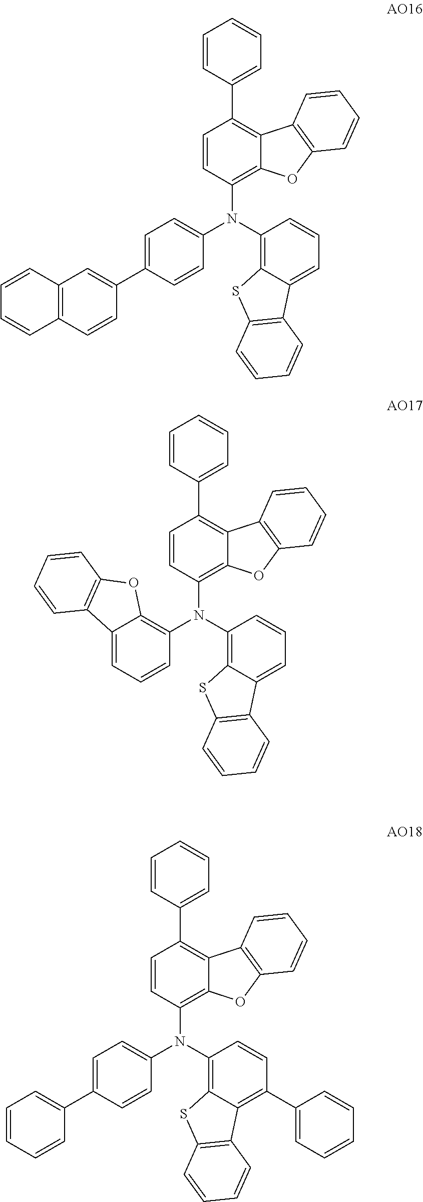 [SMILES: c1ccc(-c2ccc(N(c3ccc(-c4ccc5ccccc5c4)cc3)c3cccc4c3sc3ccccc34)c3oc4ccccc4c23)cc1 ; c1ccc(-c2ccc(N(c3cccc4c3oc3ccccc34)c3cccc4c3sc3ccccc34)c3oc4ccccc4c23)cc1 ; c1ccc(-c2ccc(N(c3ccc(-c4ccccc4)c4c3oc3ccccc34)c3ccc(-c4ccccc4)c4c3sc3ccccc34)cc2)cc1]

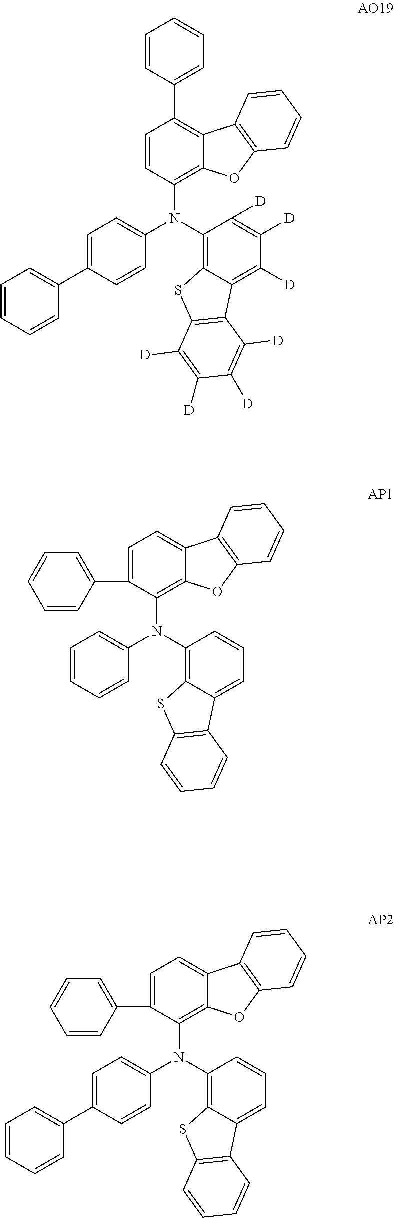 [SMILES: [2H]c1c([2H])c([2H])c2c(sc3c(N(c4ccc(-c5ccccc5)cc4)c4ccc(-c5ccccc5)c5c4oc4ccccc45)c([2H])c([2H])c([2H])c32)c1[2H] ; c1ccc(-c2ccc3c(oc4ccccc43)c2N(c2ccccc2)c2cccc3c2sc2ccccc23)cc1 ; c1ccc(-c2ccc(N(c3c(-c4ccccc4)ccc4c3oc3ccccc34)c3cccc4c3sc3ccccc34)cc2)cc1]

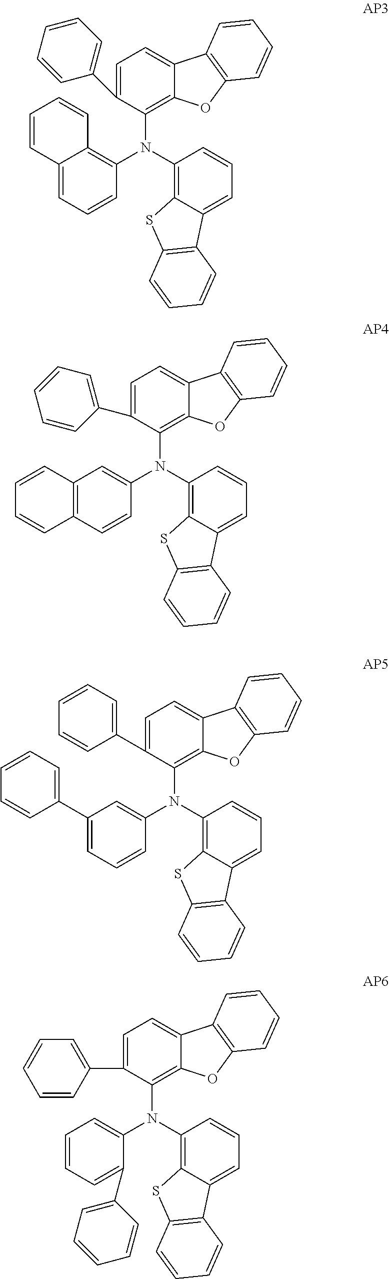

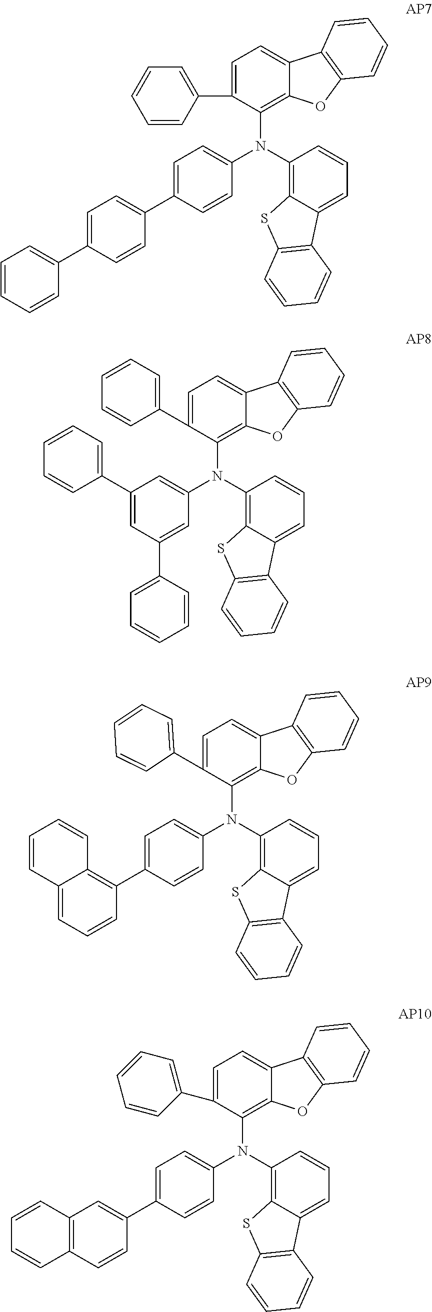

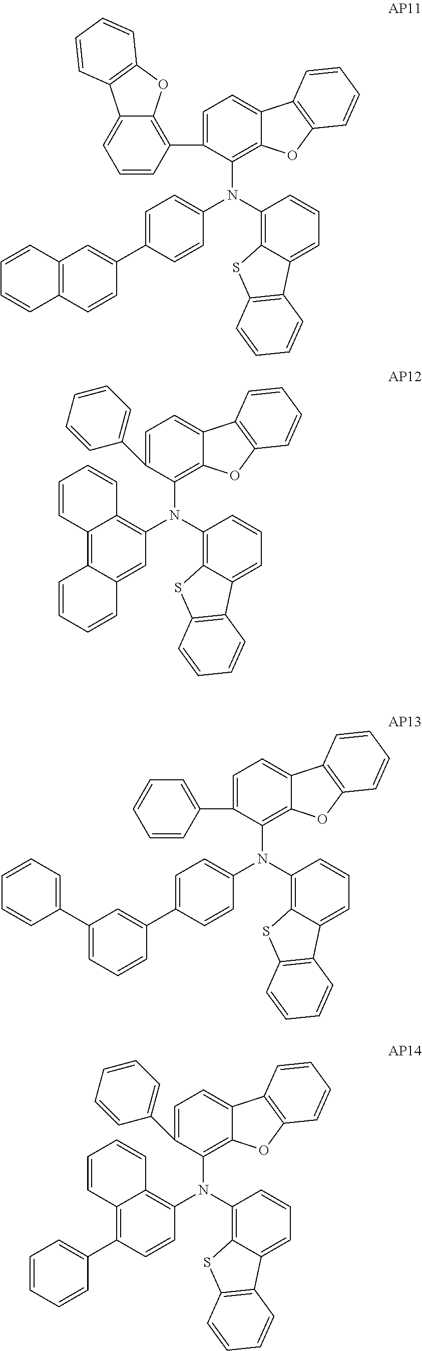

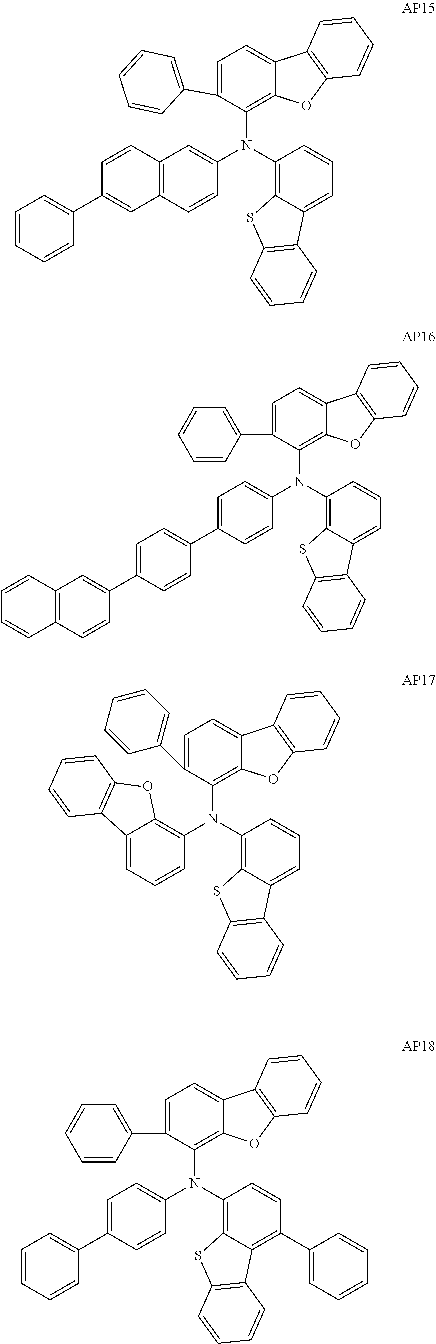 [SMILES: c1ccc(-c2ccc3cc(N(c4c(-c5ccccc5)ccc5c4oc4ccccc45)c4cccc5c4sc4ccccc45)ccc3c2)cc1 ; c1ccc(-c2ccc3c(oc4ccccc43)c2N(c2ccc(-c3ccc(-c4ccc5ccccc5c4)cc3)cc2)c2cccc3c2sc2ccccc23)cc1 ; c1ccc(-c2ccc3c(oc4ccccc43)c2N(c2cccc3c2oc2ccccc23)c2cccc3c2sc2ccccc23)cc1 ; c1ccc(-c2ccc(N(c3c(-c4ccccc4)ccc4c3oc3ccccc34)c3ccc(-c4ccccc4)c4c3sc3ccccc34)cc2)cc1]

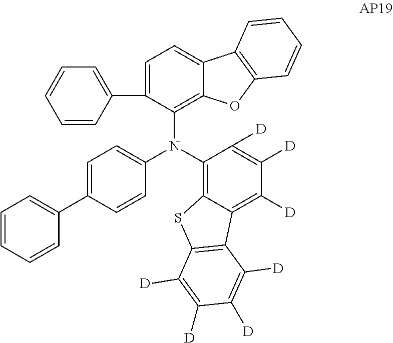 [SMILES: [2H]c1c([2H])c([2H])c2c(sc3c(N(c4ccc(-c5ccccc5)cc4)c4c(-c5ccccc5)ccc5c4oc4ccccc45)c([2H])c([2H])c([2H])c32)c1[2H]]

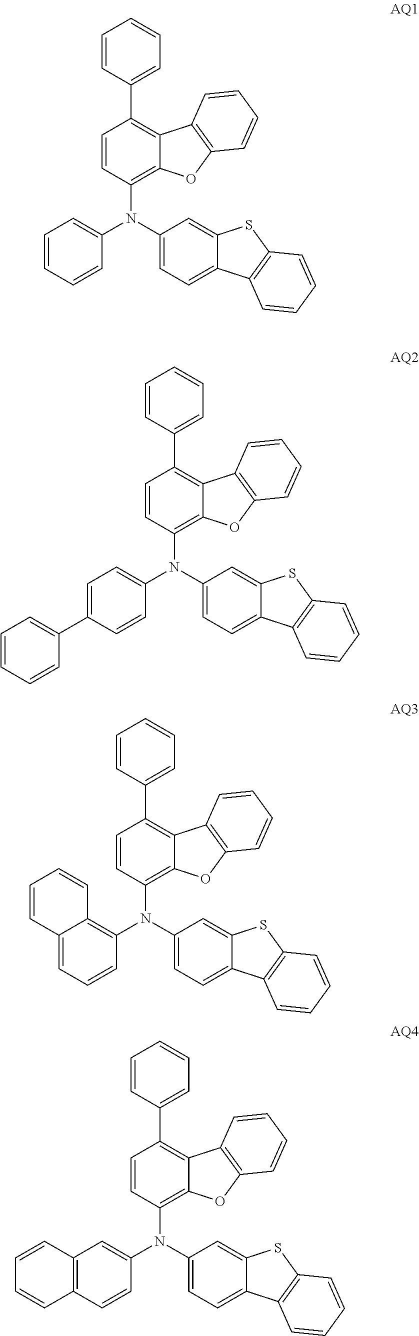 [SMILES: c1ccc(-c2ccc(N(c3ccccc3)c3ccc4c(c3)sc3ccccc34)c3oc4ccccc4c23)cc1 ; c1ccc(-c2ccc(N(c3ccc4c(c3)sc3ccccc34)c3ccc(-c4ccccc4)c4c3oc3ccccc34)cc2)cc1 ; c1ccc(-c2ccc(N(c3ccc4c(c3)sc3ccccc34)c3cccc4ccccc34)c3oc4ccccc4c23)cc1 ; c1ccc(-c2ccc(N(c3ccc4ccccc4c3)c3ccc4c(c3)sc3ccccc34)c3oc4ccccc4c23)cc1]

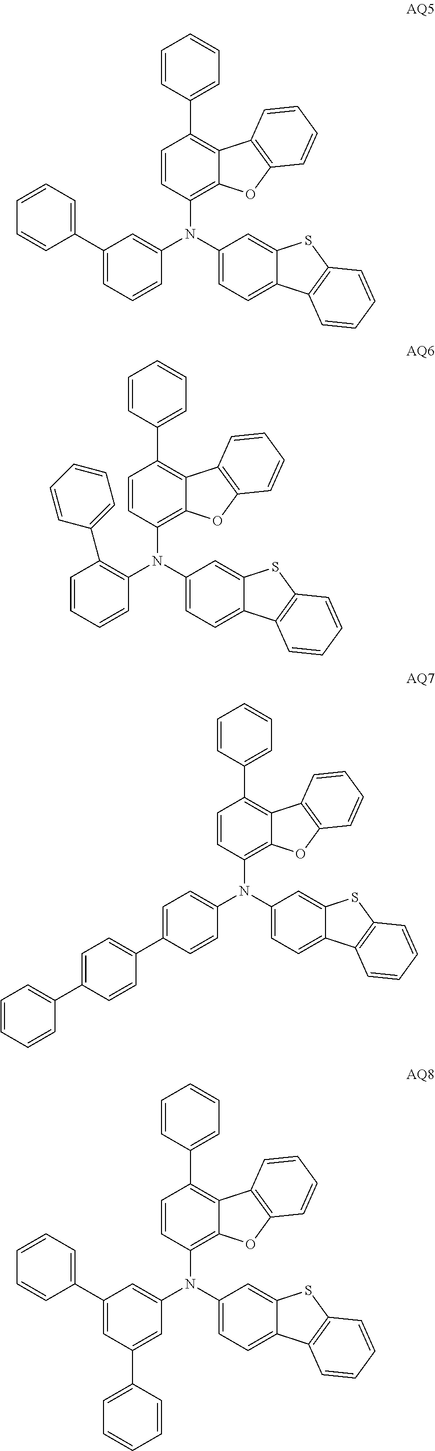 [SMILES: c1ccc(-c2cccc(N(c3ccc4c(c3)sc3ccccc34)c3ccc(-c4ccccc4)c4c3oc3ccccc34)c2)cc1 ; c1ccc(-c2ccccc2N(c2ccc3c(c2)sc2ccccc23)c2ccc(-c3ccccc3)c3c2oc2ccccc23)cc1 ; c1ccc(-c2ccc(-c3ccc(N(c4ccc5c(c4)sc4ccccc45)c4ccc(-c5ccccc5)c5c4oc4ccccc45)cc3)cc2)cc1 ; c1ccc(-c2cc(-c3ccccc3)cc(N(c3ccc4c(c3)sc3ccccc34)c3ccc(-c4ccccc4)c4c3oc3ccccc34)c2)cc1]

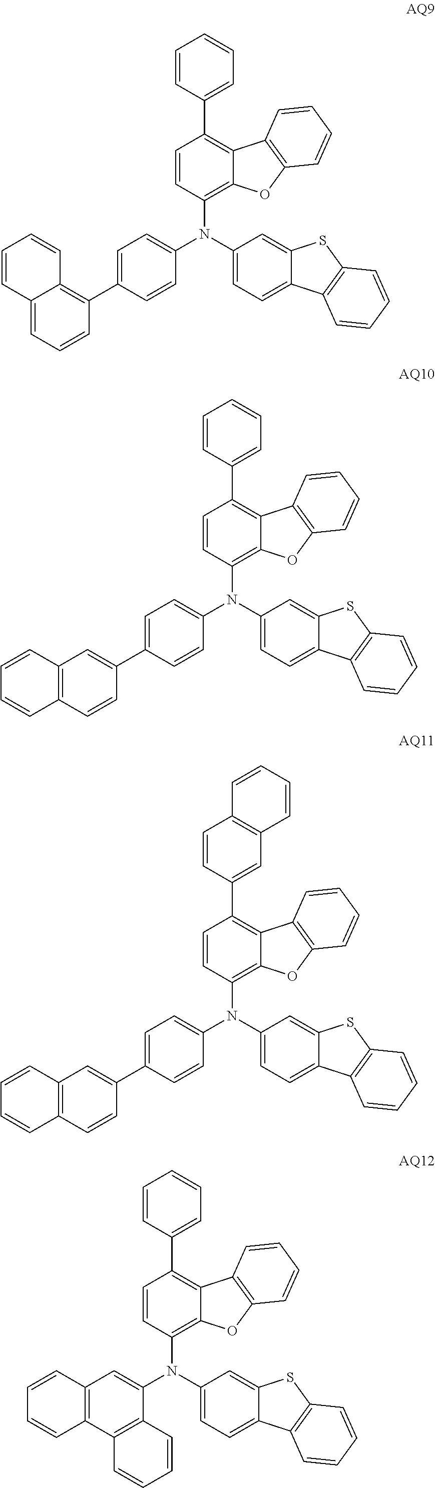 [SMILES: c1ccc(-c2ccc(N(c3ccc(-c4cccc5ccccc45)cc3)c3ccc4c(c3)sc3ccccc34)c3oc4ccccc4c23)cc1 ; c1ccc(-c2ccc(N(c3ccc(-c4ccc5ccccc5c4)cc3)c3ccc4c(c3)sc3ccccc34)c3oc4ccccc4c23)cc1 ; c1ccc2cc(-c3ccc(N(c4ccc5c(c4)sc4ccccc45)c4ccc(-c5ccc6ccccc6c5)c5c4oc4ccccc45)cc3)ccc2c1 ; c1ccc(-c2ccc(N(c3ccc4c(c3)sc3ccccc34)c3cc4ccccc4c4ccccc34)c3oc4ccccc4c23)cc1]

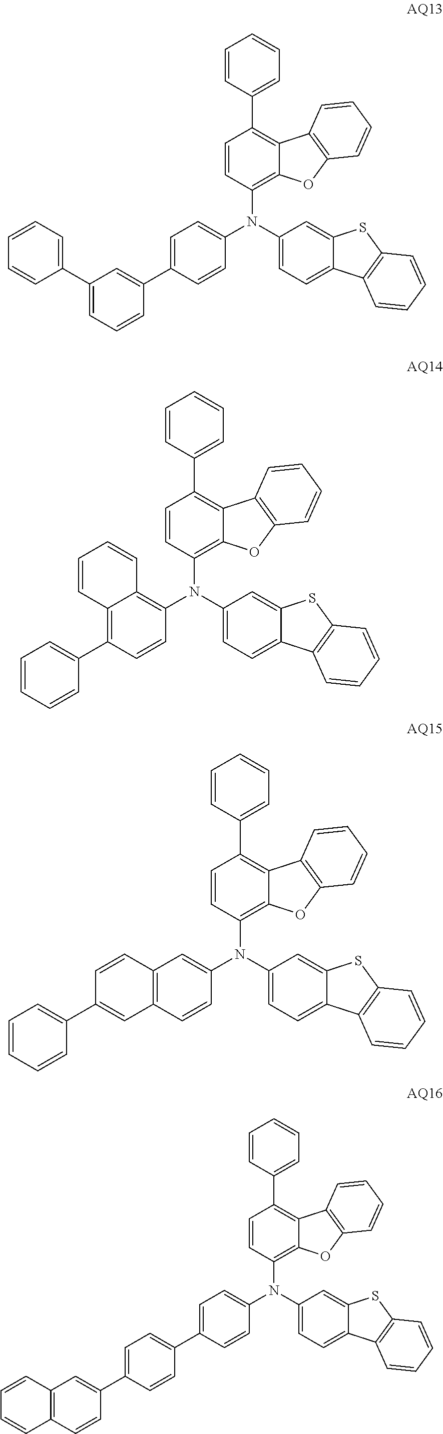 [SMILES: c1ccc(-c2cccc(-c3ccc(N(c4ccc5c(c4)sc4ccccc45)c4ccc(-c5ccccc5)c5c4oc4ccccc45)cc3)c2)cc1 ; c1ccc(-c2ccc(N(c3ccc4c(c3)sc3ccccc34)c3ccc(-c4ccccc4)c4c3oc3ccccc34)c3ccccc23)cc1 ; c1ccc(-c2ccc3cc(N(c4ccc5c(c4)sc4ccccc45)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc4c(c3)sc3ccccc34)c3oc4ccccc4c23)cc1]

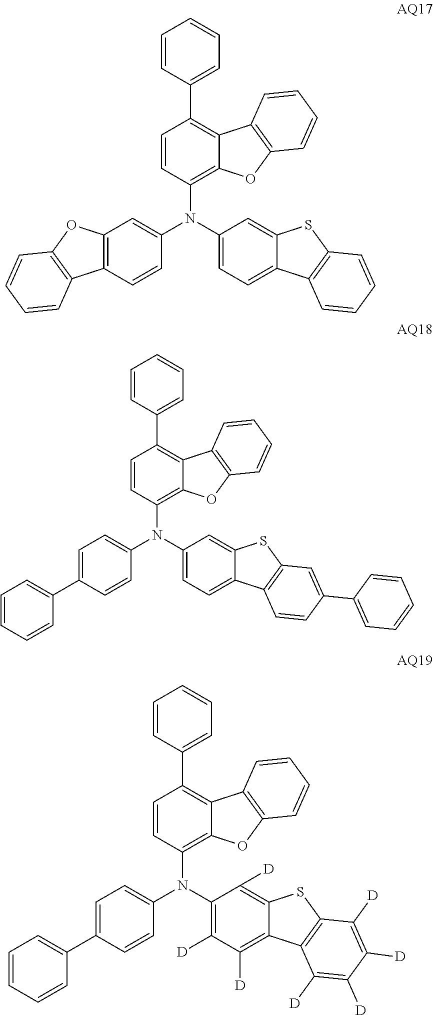 [SMILES: c1ccc(-c2ccc(N(c3ccc4c(c3)oc3ccccc34)c3ccc4c(c3)sc3ccccc34)c3oc4ccccc4c23)cc1 ; c1ccc(-c2ccc(N(c3ccc4c(c3)sc3cc(-c5ccccc5)ccc34)c3ccc(-c4ccccc4)c4c3oc3ccccc34)cc2)cc1 ; [2H]c1c([2H])c([2H])c2c(sc3c([2H])c(N(c4ccc(-c5ccccc5)cc4)c4ccc(-c5ccccc5)c5c4oc4ccccc45)c([2H])c([2H])c32)c1[2H]]

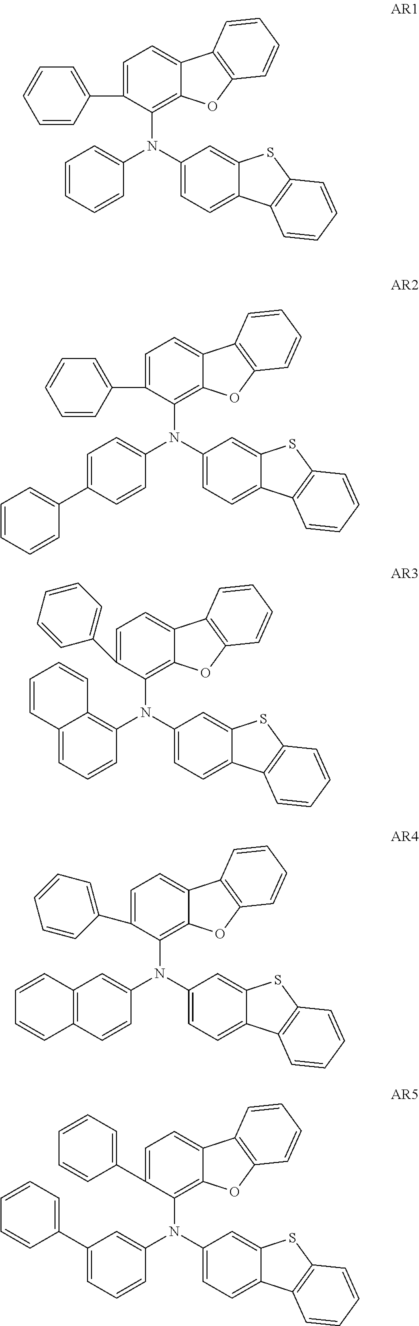

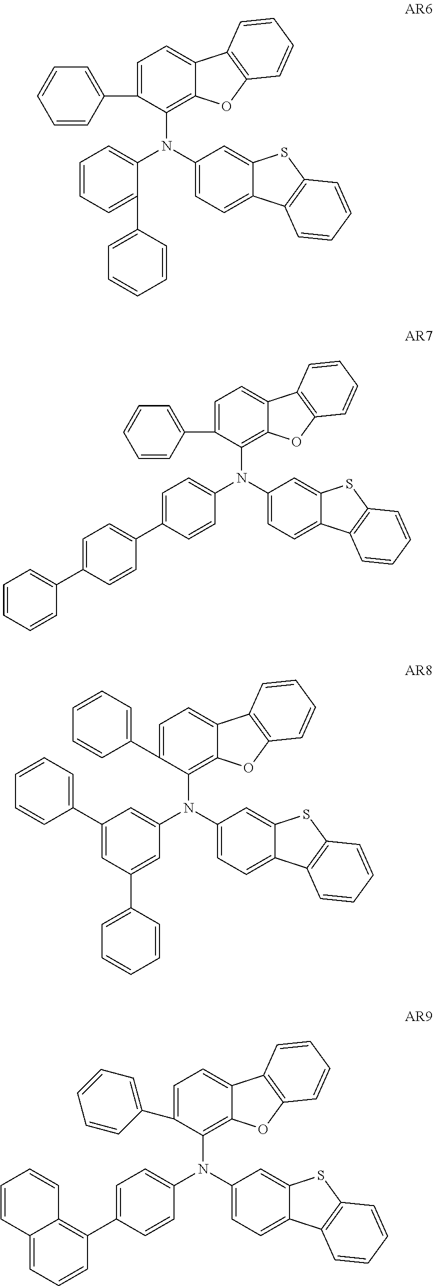 [SMILES: c1ccc(-c2ccccc2N(c2ccc3c(c2)sc2ccccc23)c2c(-c3ccccc3)ccc3c2oc2ccccc23)cc1 ; c1ccc(-c2ccc(-c3ccc(N(c4ccc5c(c4)sc4ccccc45)c4c(-c5ccccc5)ccc5c4oc4ccccc45)cc3)cc2)cc1 ; c1ccc(-c2cc(-c3ccccc3)cc(N(c3ccc4c(c3)sc3ccccc34)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc3c(c2)sc2ccccc23)cc1]

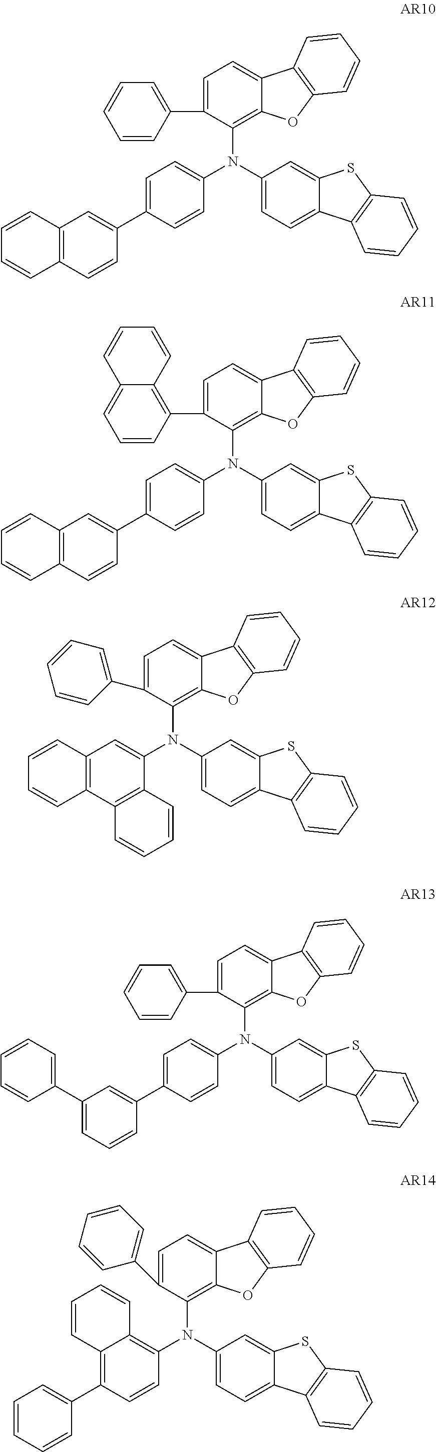 [SMILES: c1ccc(-c2ccc3c(oc4ccccc43)c2N(c2ccc(-c3ccc4ccccc4c3)cc2)c2ccc3c(c2)sc2ccccc23)cc1 ; c1ccc2cc(-c3ccc(N(c4ccc5c(c4)sc4ccccc45)c4c(-c5cccc6ccccc56)ccc5c4oc4ccccc45)cc3)ccc2c1 ; c1ccc(-c2ccc3c(oc4ccccc43)c2N(c2ccc3c(c2)sc2ccccc23)c2cc3ccccc3c3ccccc23)cc1 ; c1ccc(-c2cccc(-c3ccc(N(c4ccc5c(c4)sc4ccccc45)c4c(-c5ccccc5)ccc5c4oc4ccccc45)cc3)c2)cc1 ; c1ccc(-c2ccc3c(oc4ccccc43)c2N(c2ccc3c(c2)sc2ccccc23)c2ccc(-c3ccccc3)c3ccccc23)cc1]

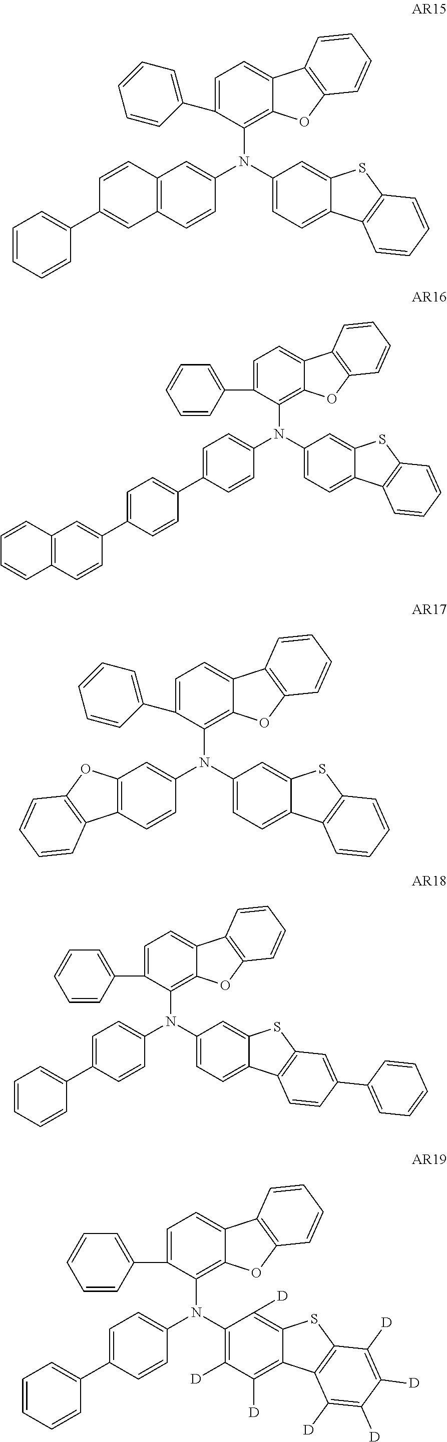 [SMILES: c1ccc(-c2ccc3cc(N(c4ccc5c(c4)sc4ccccc45)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc3c(c2)sc2ccccc23)cc1 ; c1ccc(-c2ccc3c(oc4ccccc43)c2N(c2ccc3c(c2)oc2ccccc23)c2ccc3c(c2)sc2ccccc23)cc1 ; c1ccc(-c2ccc(N(c3ccc4c(c3)sc3cc(-c5ccccc5)ccc34)c3c(-c4ccccc4)ccc4c3oc3ccccc34)cc2)cc1 ; [2H]c1c([2H])c([2H])c2c(sc3c([2H])c(N(c4ccc(-c5ccccc5)cc4)c4c(-c5ccccc5)ccc5c4oc4ccccc45)c([2H])c([2H])c32)c1[2H]]

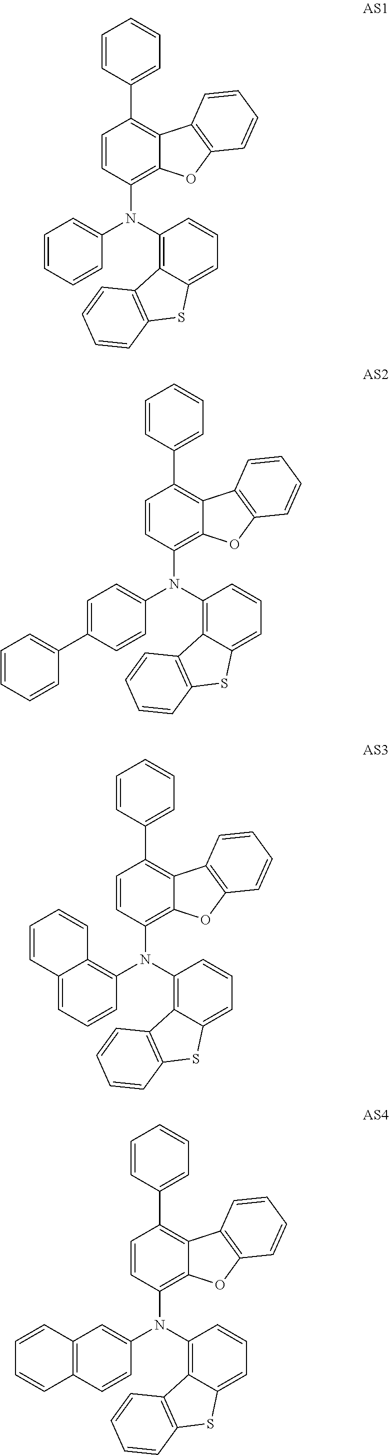

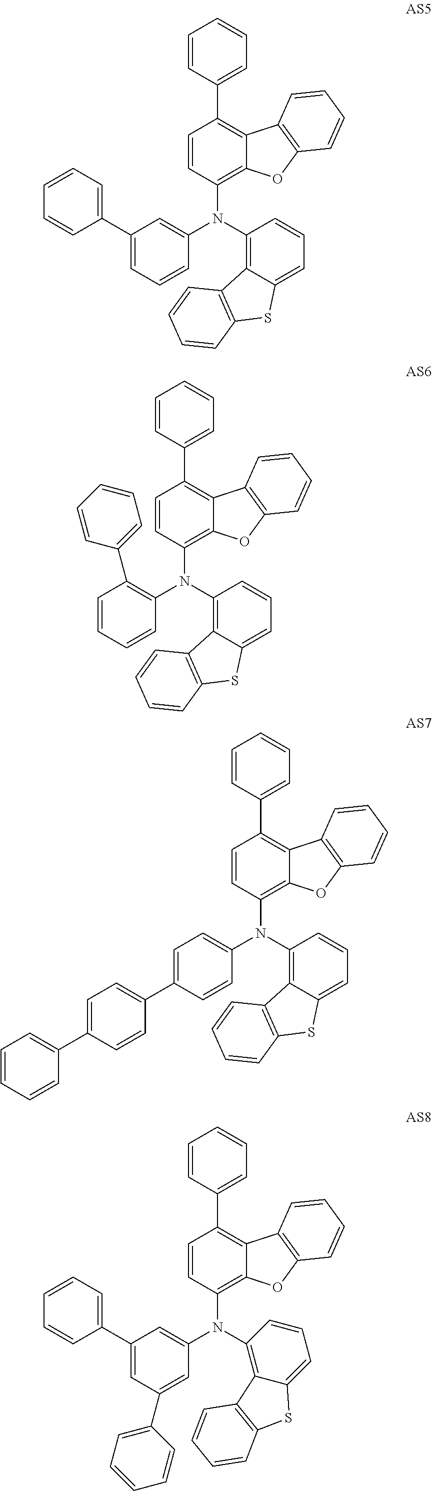

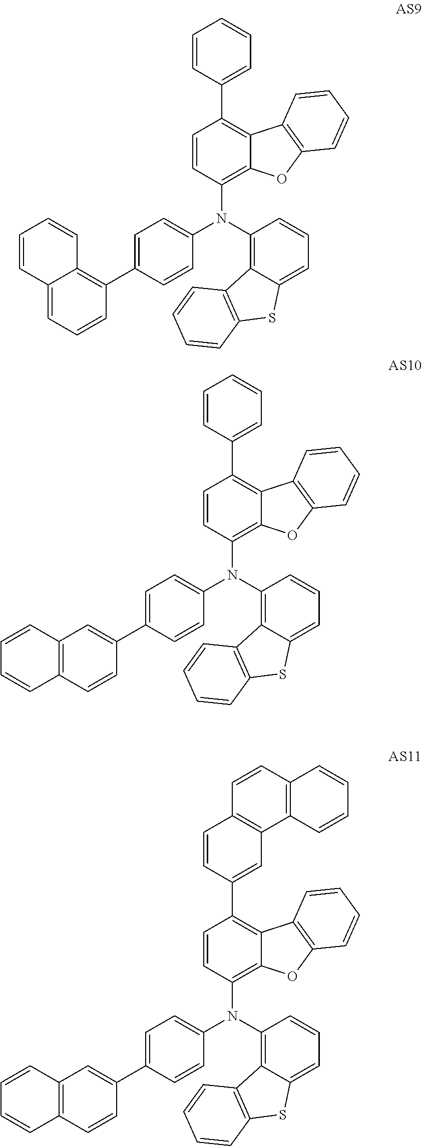

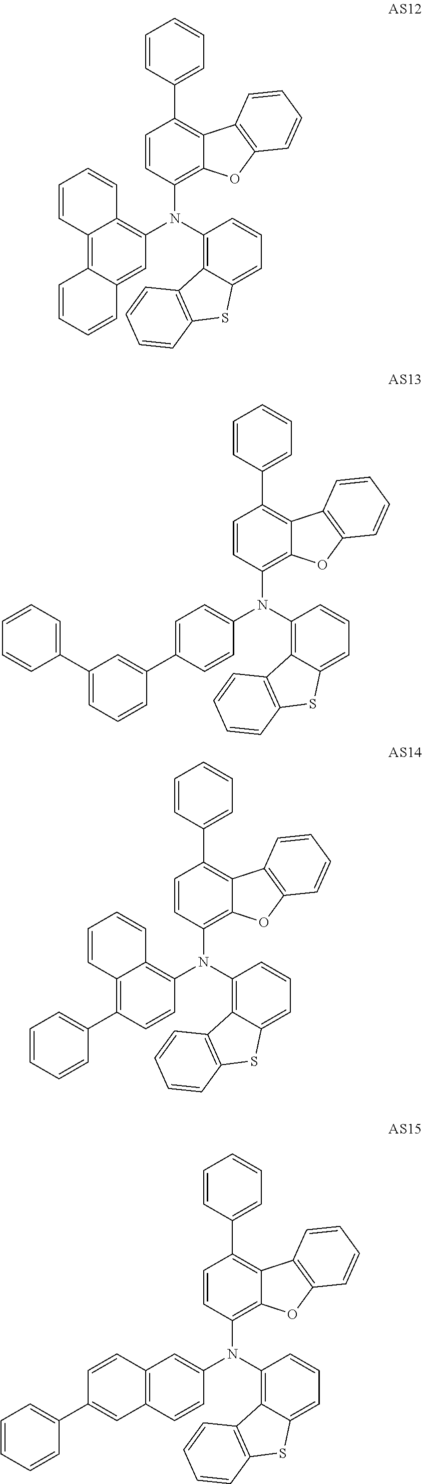

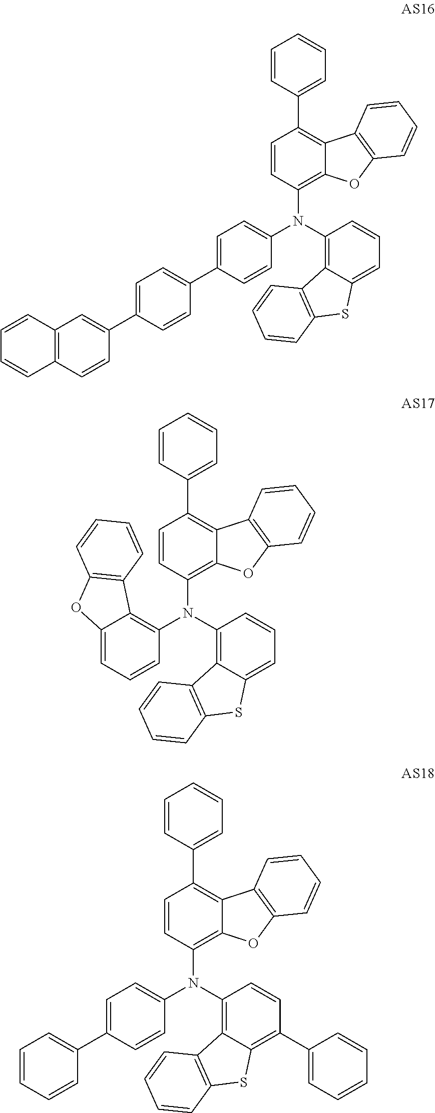

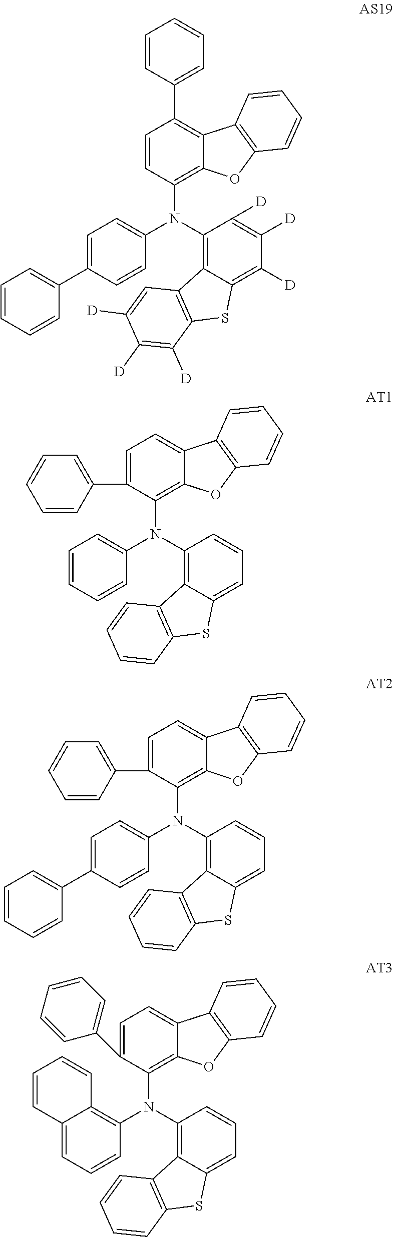 [SMILES: [2H]c1cc2c(sc3c([2H])c([2H])c([2H])c(N(c4ccc(-c5ccccc5)cc4)c4ccc(-c5ccccc5)c5c4oc4ccccc45)c32)c([2H])c1[2H] ; c1ccc(-c2ccc3c(oc4ccccc43)c2N(c2ccccc2)c2cccc3sc4ccccc4c23)cc1 ; c1ccc(-c2ccc(N(c3c(-c4ccccc4)ccc4c3oc3ccccc34)c3cccc4sc5ccccc5c34)cc2)cc1 ; c1ccc(-c2ccc3c(oc4ccccc43)c2N(c2cccc3ccccc23)c2cccc3sc4ccccc4c23)cc1]

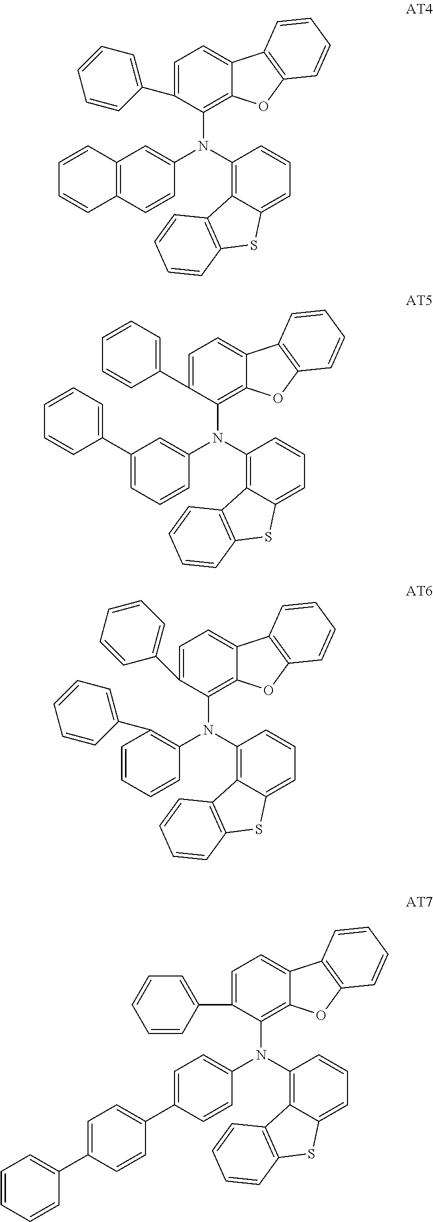 [SMILES: c1ccc(-c2ccc3c(oc4ccccc43)c2N(c2ccc3ccccc3c2)c2cccc3sc4ccccc4c23)cc1 ; c1ccc(-c2cccc(N(c3c(-c4ccccc4)ccc4c3oc3ccccc34)c3cccc4sc5ccccc5c34)c2)cc1 ; c1ccc(-c2ccccc2N(c2c(-c3ccccc3)ccc3c2oc2ccccc23)c2cccc3sc4ccccc4c23)cc1 ; c1ccc(-c2ccc(-c3ccc(N(c4c(-c5ccccc5)ccc5c4oc4ccccc45)c4cccc5sc6ccccc6c45)cc3)cc2)cc1]

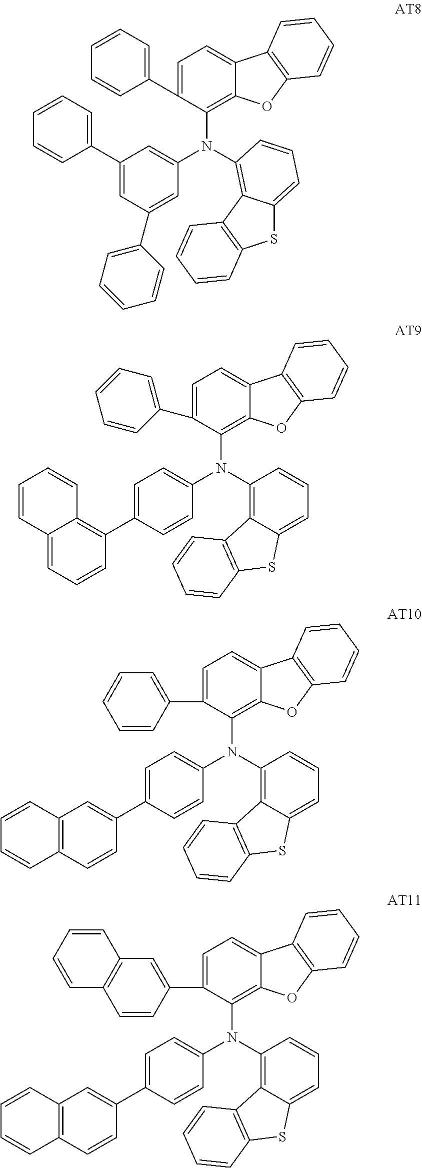 [SMILES: c1ccc(-c2cc(-c3ccccc3)cc(N(c3c(-c4ccccc4)ccc4c3oc3ccccc34)c3cccc4sc5ccccc5c34)c2)cc1 ; c1ccc(-c2ccc3c(oc4ccccc43)c2N(c2ccc(-c3cccc4ccccc34)cc2)c2cccc3sc4ccccc4c23)cc1 ; c1ccc(-c2ccc3c(oc4ccccc43)c2N(c2ccc(-c3ccc4ccccc4c3)cc2)c2cccc3sc4ccccc4c23)cc1 ; c1ccc2cc(-c3ccc(N(c4c(-c5ccc6ccccc6c5)ccc5c4oc4ccccc45)c4cccc5sc6ccccc6c45)cc3)ccc2c1]

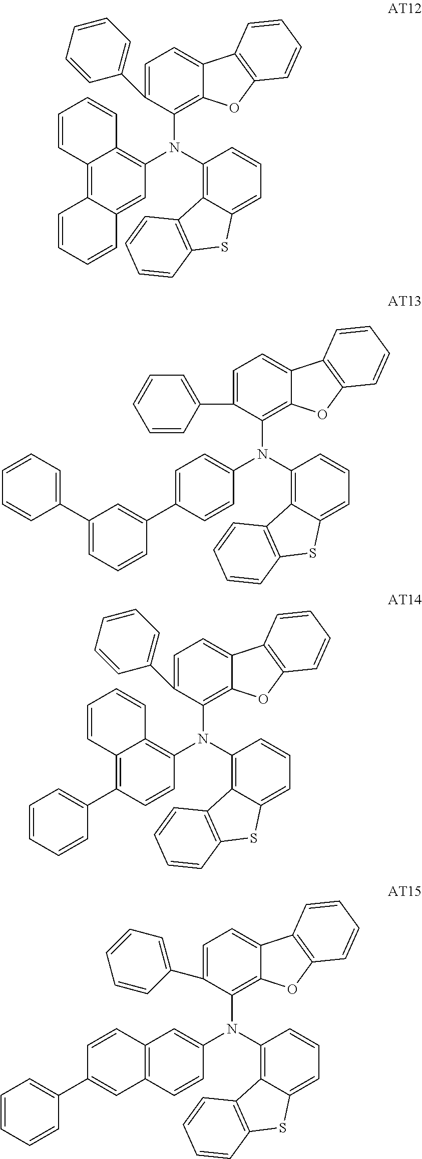

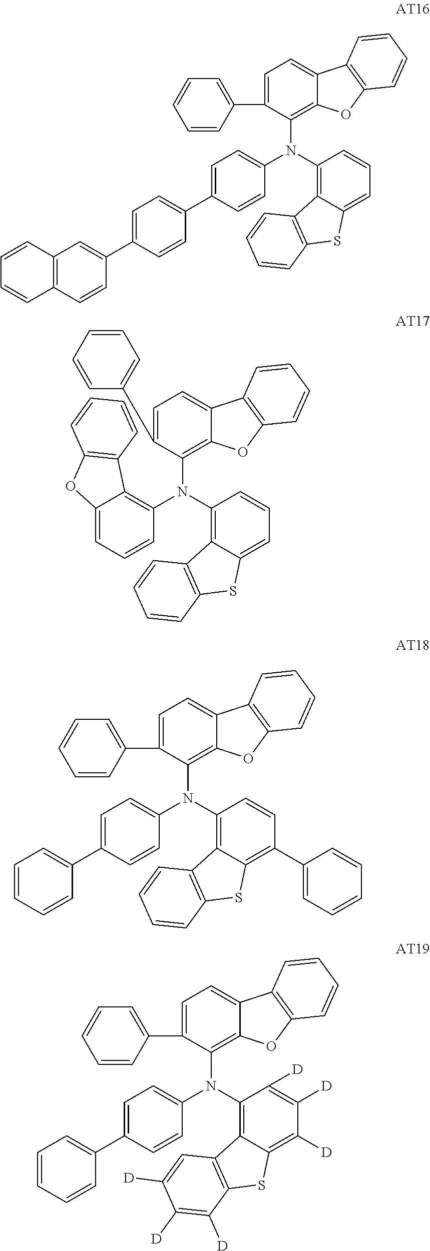

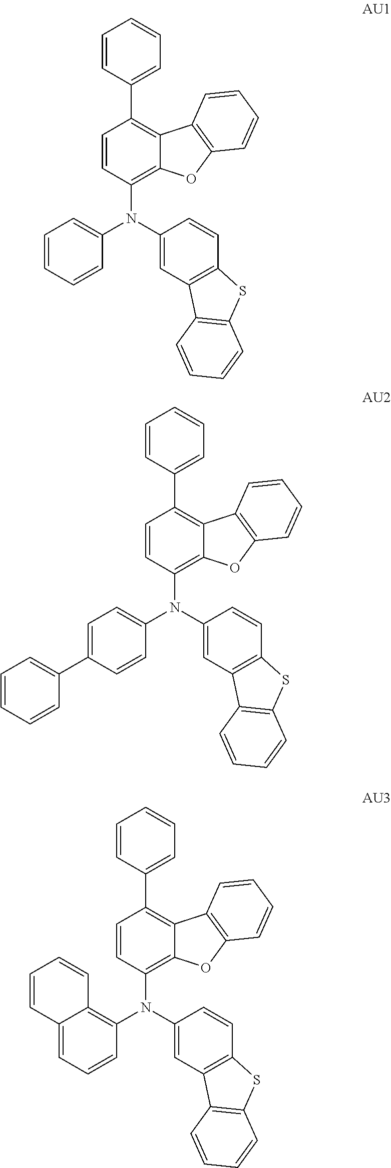

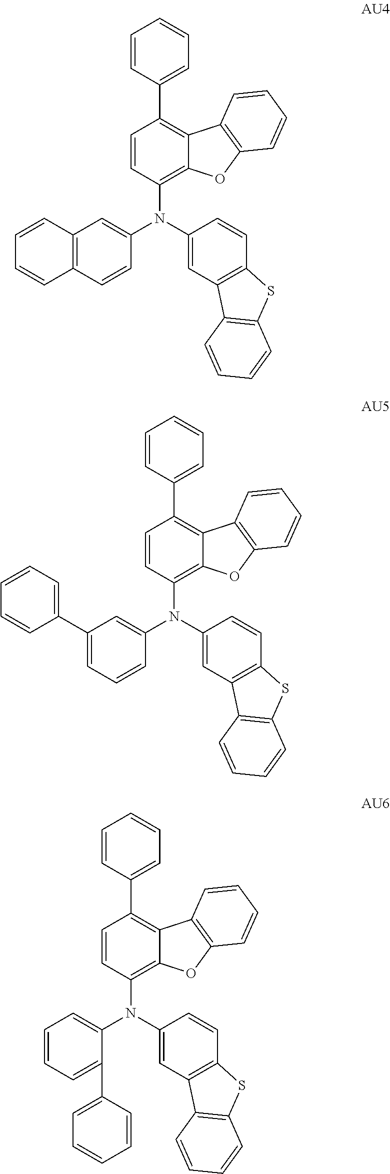 [SMILES: c1ccc(-c2ccc(N(c3ccc4ccccc4c3)c3ccc4sc5ccccc5c4c3)c3oc4ccccc4c23)cc1 ; c1ccc(-c2cccc(N(c3ccc4sc5ccccc5c4c3)c3ccc(-c4ccccc4)c4c3oc3ccccc34)c2)cc1 ; c1ccc(-c2ccccc2N(c2ccc3sc4ccccc4c3c2)c2ccc(-c3ccccc3)c3c2oc2ccccc23)cc1]

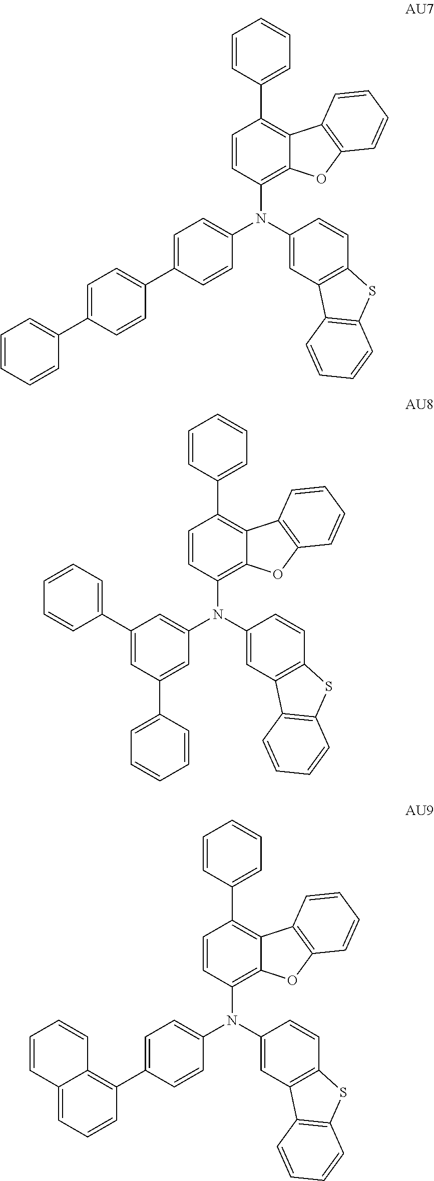 [SMILES: c1ccc(-c2ccc(-c3ccc(N(c4ccc5sc6ccccc6c5c4)c4ccc(-c5ccccc5)c5c4oc4ccccc45)cc3)cc2)cc1 ; c1ccc(-c2cc(-c3ccccc3)cc(N(c3ccc4sc5ccccc5c4c3)c3ccc(-c4ccccc4)c4c3oc3ccccc34)c2)cc1 ; c1ccc(-c2ccc(N(c3ccc(-c4cccc5ccccc45)cc3)c3ccc4sc5ccccc5c4c3)c3oc4ccccc4c23)cc1]

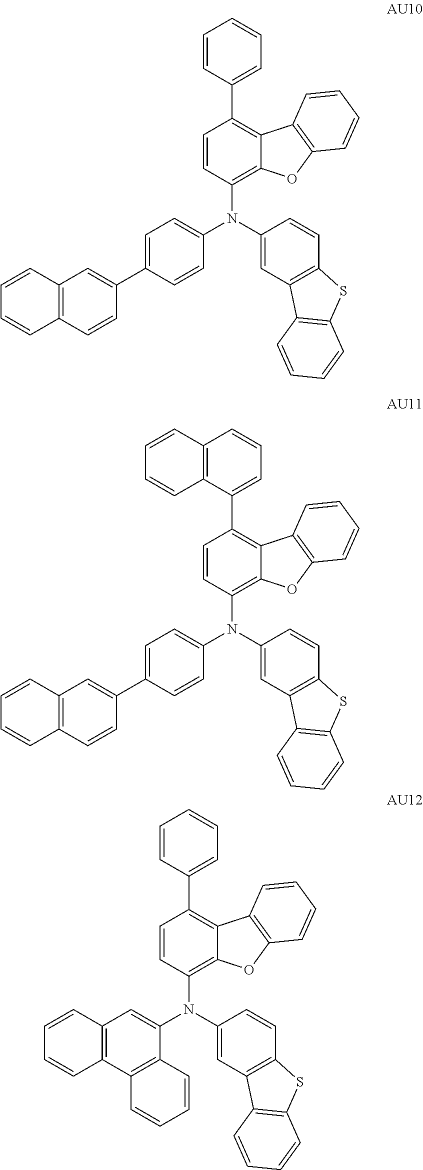 [SMILES: c1ccc(-c2ccc(N(c3ccc(-c4ccc5ccccc5c4)cc3)c3ccc4sc5ccccc5c4c3)c3oc4ccccc4c23)cc1 ; c1ccc2cc(-c3ccc(N(c4ccc5sc6ccccc6c5c4)c4ccc(-c5cccc6ccccc56)c5c4oc4ccccc45)cc3)ccc2c1 ; c1ccc(-c2ccc(N(c3ccc4sc5ccccc5c4c3)c3cc4ccccc4c4ccccc34)c3oc4ccccc4c23)cc1]

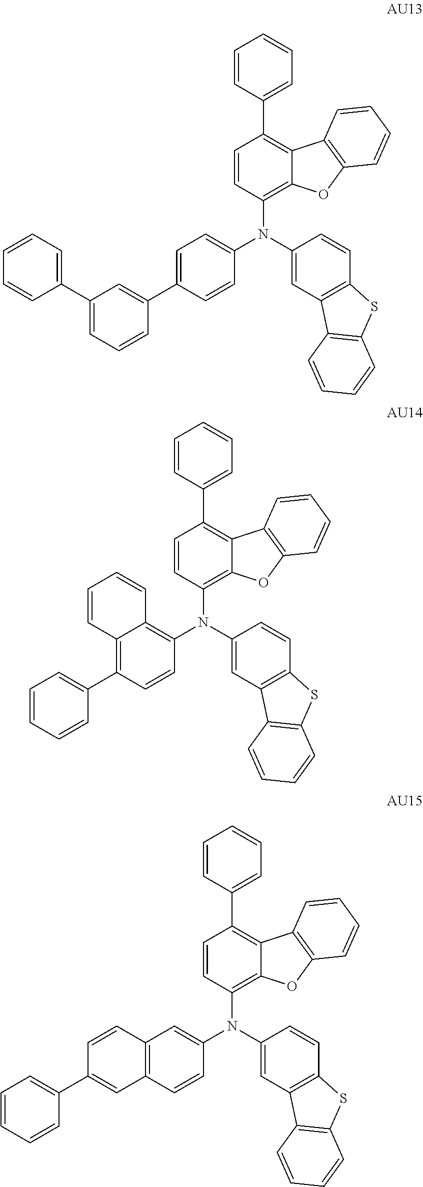 [SMILES: c1ccc(-c2cccc(-c3ccc(N(c4ccc5sc6ccccc6c5c4)c4ccc(-c5ccccc5)c5c4oc4ccccc45)cc3)c2)cc1 ; c1ccc(-c2ccc(N(c3ccc4sc5ccccc5c4c3)c3ccc(-c4ccccc4)c4c3oc3ccccc34)c3ccccc23)cc1 ; c1ccc(-c2ccc3cc(N(c4ccc5sc6ccccc6c5c4)c4ccc(-c5ccccc5)c5c4oc4ccccc45)ccc3c2)cc1]

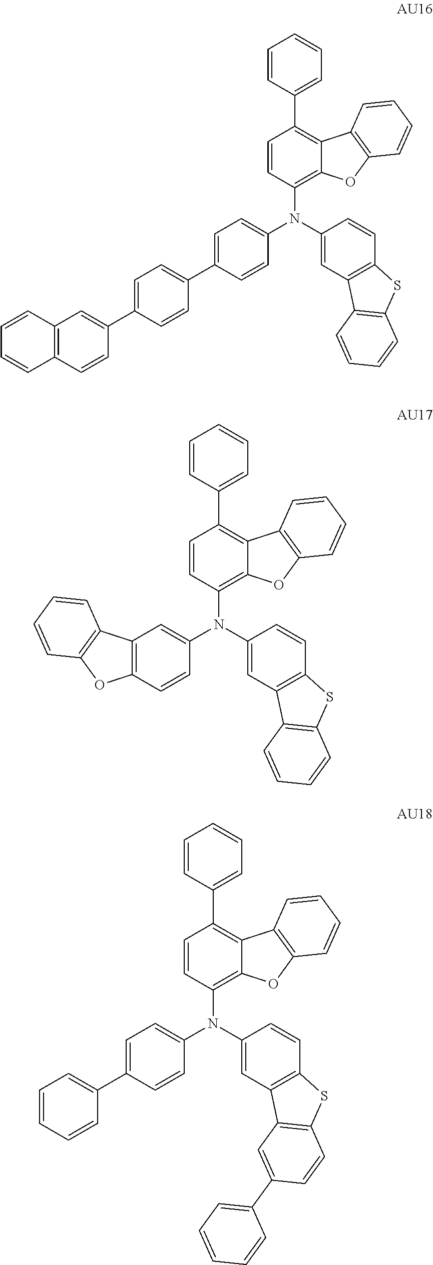 [SMILES: c1ccc(-c2ccc(N(c3ccc(-c4ccc(-c5ccc6ccccc6c5)cc4)cc3)c3ccc4sc5ccccc5c4c3)c3oc4ccccc4c23)cc1 ; c1ccc(-c2ccc(N(c3ccc4oc5ccccc5c4c3)c3ccc4sc5ccccc5c4c3)c3oc4ccccc4c23)cc1 ; c1ccc(-c2ccc(N(c3ccc4sc5ccc(-c6ccccc6)cc5c4c3)c3ccc(-c4ccccc4)c4c3oc3ccccc34)cc2)cc1]

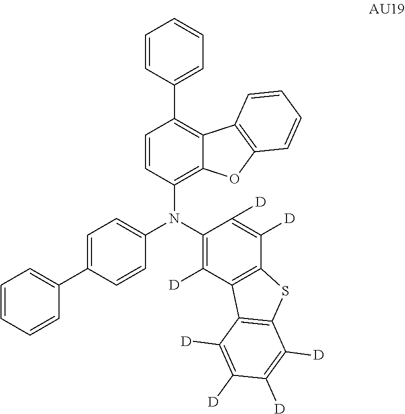 [SMILES: [2H]c1c([2H])c([2H])c2c(sc3c([2H])c([2H])c(N(c4ccc(-c5ccccc5)cc4)c4ccc(-c5ccccc5)c5c4oc4ccccc45)c([2H])c32)c1[2H]]

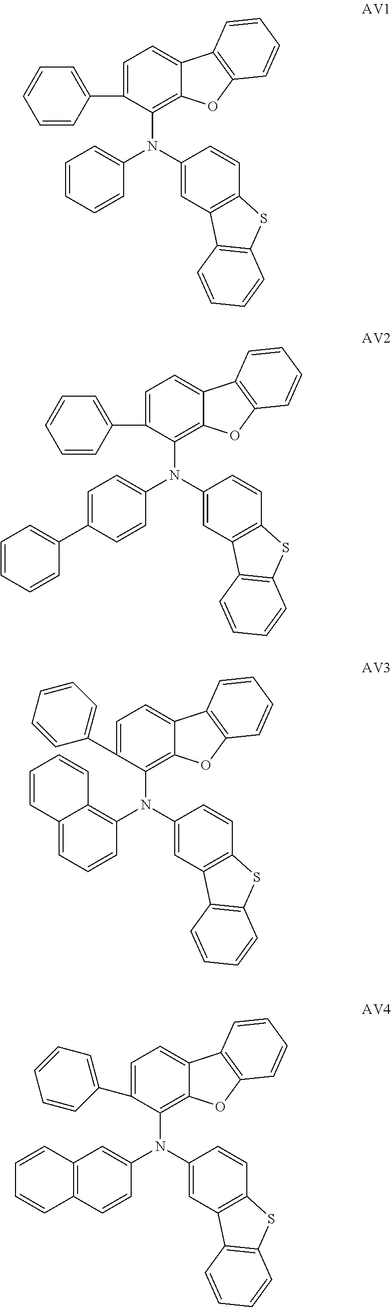

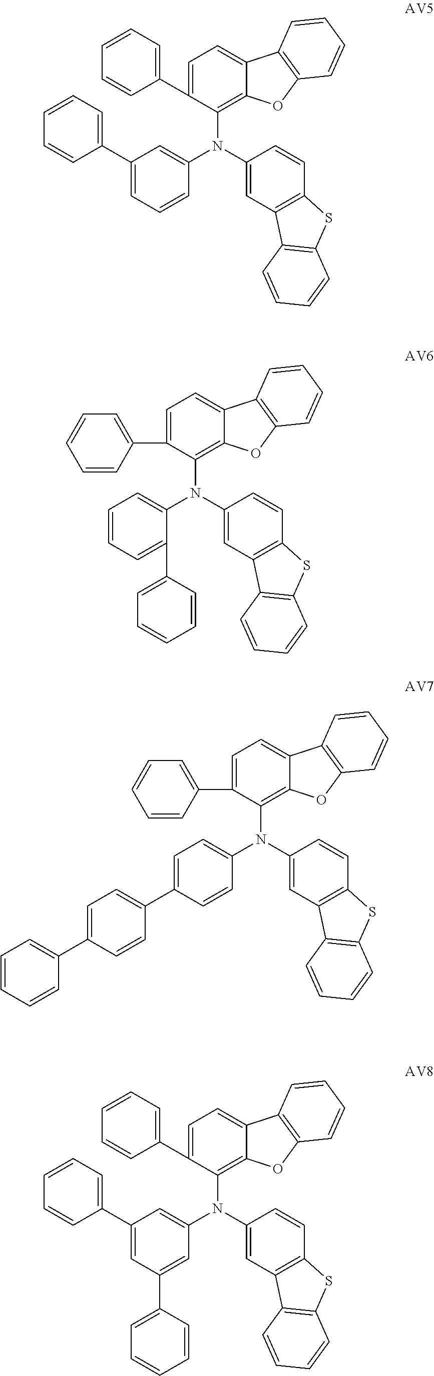 [SMILES: c1ccc(-c2cccc(N(c3ccc4sc5ccccc5c4c3)c3c(-c4ccccc4)ccc4c3oc3ccccc34)c2)cc1 ; c1ccc(-c2ccccc2N(c2ccc3sc4ccccc4c3c2)c2c(-c3ccccc3)ccc3c2oc2ccccc23)cc1 ; c1ccc(-c2ccc(-c3ccc(N(c4ccc5sc6ccccc6c5c4)c4c(-c5ccccc5)ccc5c4oc4ccccc45)cc3)cc2)cc1 ; c1ccc(-c2cc(-c3ccccc3)cc(N(c3ccc4sc5ccccc5c4c3)c3c(-c4ccccc4)ccc4c3oc3ccccc34)c2)cc1]

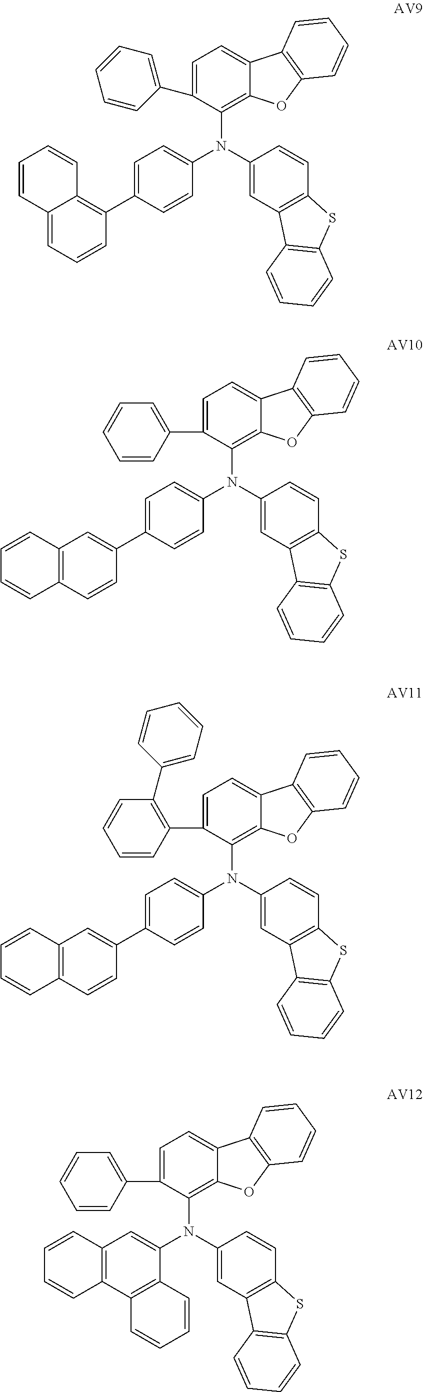 [SMILES: c1ccc(-c2ccc3c(oc4ccccc43)c2N(c2ccc(-c3cccc4ccccc34)cc2)c2ccc3sc4ccccc4c3c2)cc1 ; c1ccc(-c2ccc3c(oc4ccccc43)c2N(c2ccc(-c3ccc4ccccc4c3)cc2)c2ccc3sc4ccccc4c3c2)cc1 ; c1ccc(-c2ccccc2-c2ccc3c(oc4ccccc43)c2N(c2ccc(-c3ccc4ccccc4c3)cc2)c2ccc3sc4ccccc4c3c2)cc1 ; c1ccc(-c2ccc3c(oc4ccccc43)c2N(c2ccc3sc4ccccc4c3c2)c2cc3ccccc3c3ccccc23)cc1]

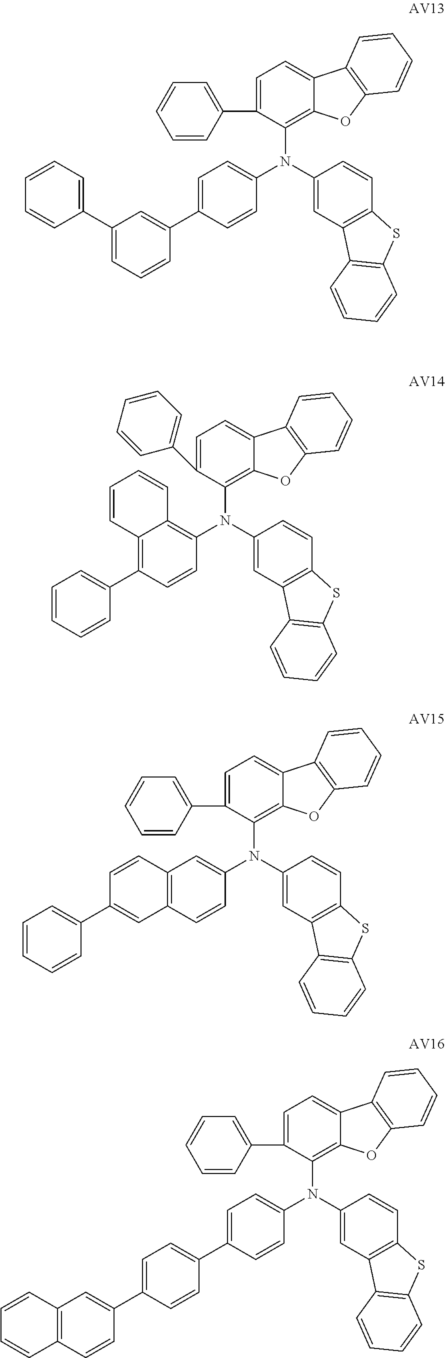 [SMILES: c1ccc(-c2cccc(-c3ccc(N(c4ccc5sc6ccccc6c5c4)c4c(-c5ccccc5)ccc5c4oc4ccccc45)cc3)c2)cc1 ; c1ccc(-c2ccc3c(oc4ccccc43)c2N(c2ccc3sc4ccccc4c3c2)c2ccc(-c3ccccc3)c3ccccc23)cc1 ; c1ccc(-c2ccc3cc(N(c4ccc5sc6ccccc6c5c4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc3sc4ccccc4c3c2)cc1]

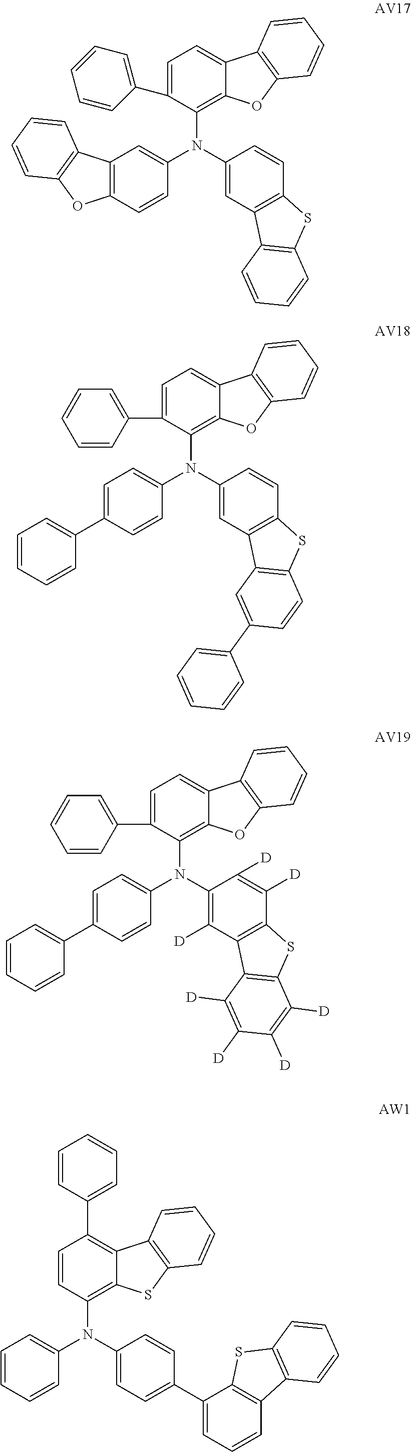

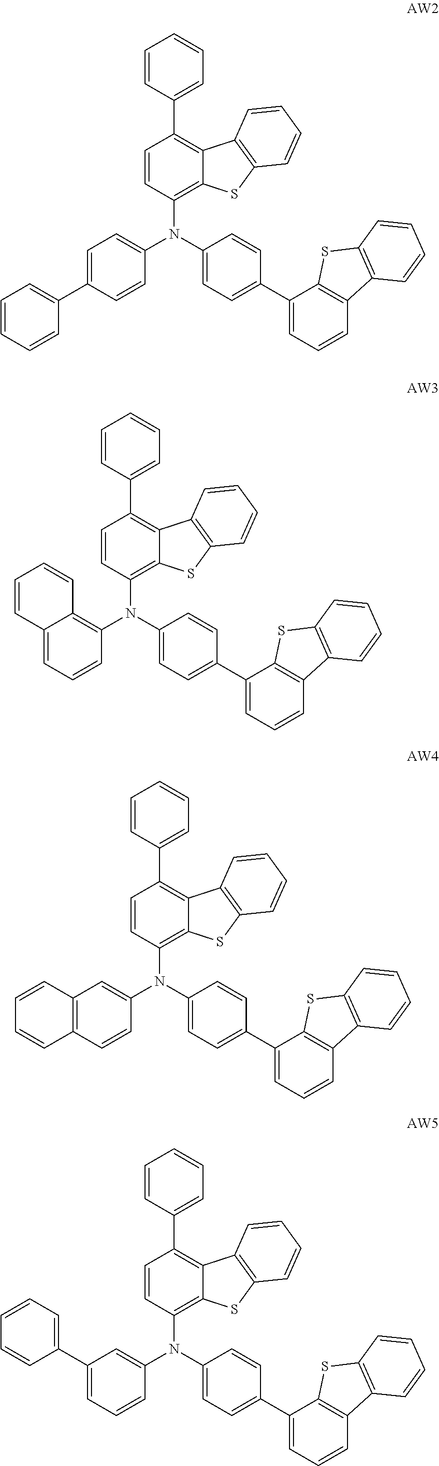 [SMILES: c1ccc(-c2ccc(N(c3ccc(-c4cccc5c4sc4ccccc45)cc3)c3ccc(-c4ccccc4)c4c3sc3ccccc34)cc2)cc1 ; c1ccc(-c2ccc(N(c3ccc(-c4cccc5c4sc4ccccc45)cc3)c3cccc4ccccc34)c3sc4ccccc4c23)cc1 ; c1ccc(-c2ccc(N(c3ccc(-c4cccc5c4sc4ccccc45)cc3)c3ccc4ccccc4c3)c3sc4ccccc4c23)cc1 ; c1ccc(-c2cccc(N(c3ccc(-c4cccc5c4sc4ccccc45)cc3)c3ccc(-c4ccccc4)c4c3sc3ccccc34)c2)cc1]

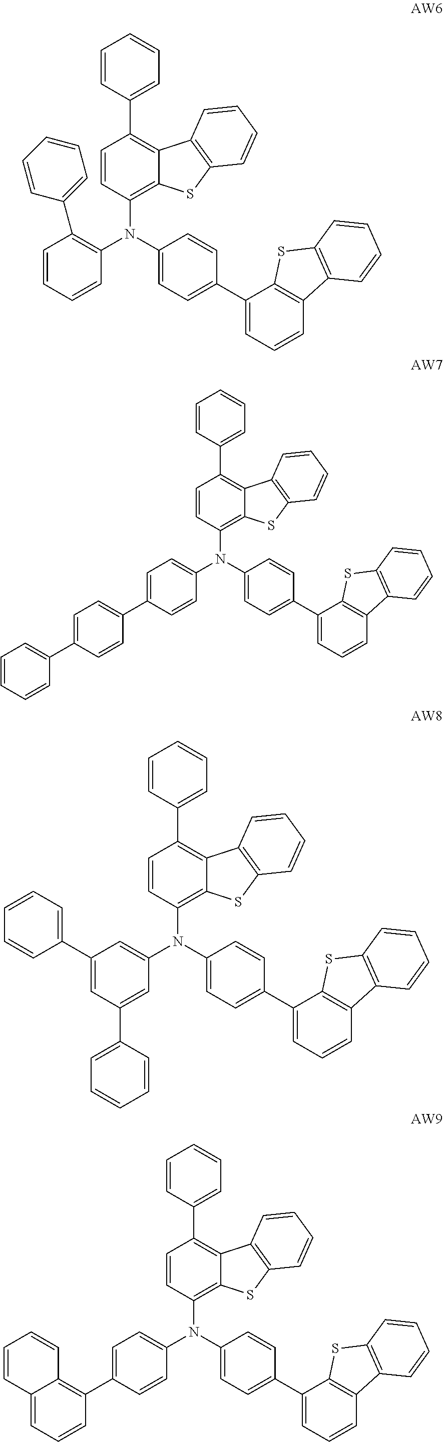 [SMILES: c1ccc(-c2ccccc2N(c2ccc(-c3cccc4c3sc3ccccc34)cc2)c2ccc(-c3ccccc3)c3c2sc2ccccc23)cc1 ; c1ccc(-c2ccc(-c3ccc(N(c4ccc(-c5cccc6c5sc5ccccc56)cc4)c4ccc(-c5ccccc5)c5c4sc4ccccc45)cc3)cc2)cc1 ; c1ccc(-c2cc(-c3ccccc3)cc(N(c3ccc(-c4cccc5c4sc4ccccc45)cc3)c3ccc(-c4ccccc4)c4c3sc3ccccc34)c2)cc1 ; c1ccc(-c2ccc(N(c3ccc(-c4cccc5ccccc45)cc3)c3ccc(-c4cccc5c4sc4ccccc45)cc3)c3sc4ccccc4c23)cc1]

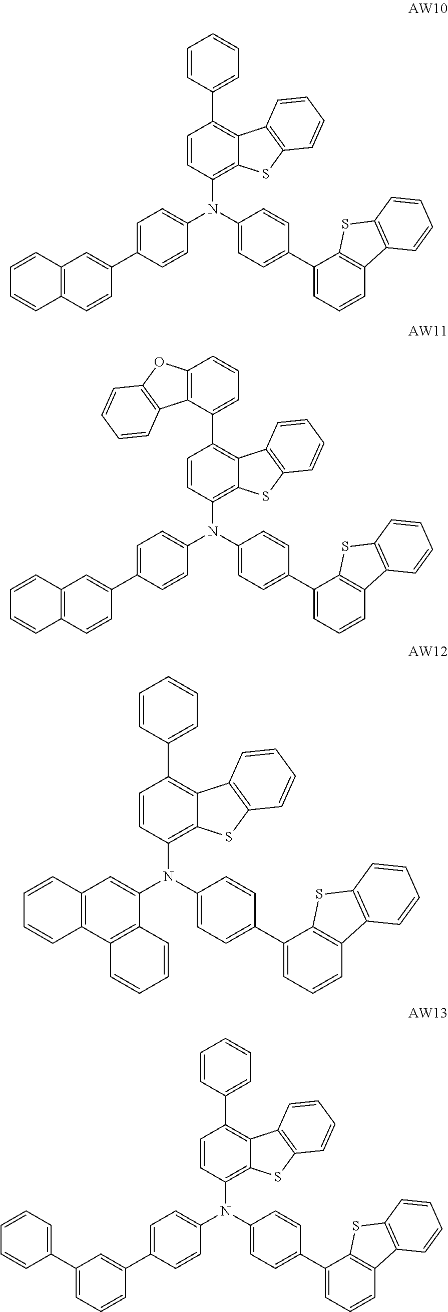 [SMILES: c1ccc(-c2ccc(N(c3ccc(-c4ccc5ccccc5c4)cc3)c3ccc(-c4cccc5c4sc4ccccc45)cc3)c3sc4ccccc4c23)cc1 ; c1ccc2cc(-c3ccc(N(c4ccc(-c5cccc6c5sc5ccccc56)cc4)c4ccc(-c5cccc6oc7ccccc7c56)c5c4sc4ccccc45)cc3)ccc2c1 ; c1ccc(-c2ccc(N(c3ccc(-c4cccc5c4sc4ccccc45)cc3)c3cc4ccccc4c4ccccc34)c3sc4ccccc4c23)cc1 ; c1ccc(-c2cccc(-c3ccc(N(c4ccc(-c5cccc6c5sc5ccccc56)cc4)c4ccc(-c5ccccc5)c5c4sc4ccccc45)cc3)c2)cc1]

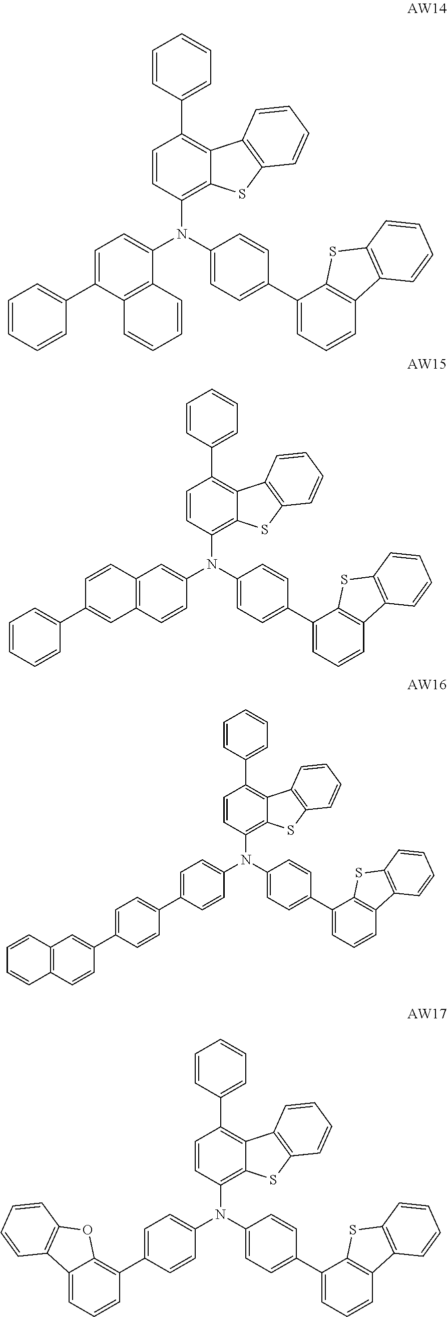 [SMILES: c1ccc(-c2ccc(N(c3ccc(-c4cccc5c4sc4ccccc45)cc3)c3ccc(-c4ccccc4)c4c3sc3ccccc34)c3ccccc23)cc1 ; c1ccc(-c2ccc3cc(N(c4ccc(-c5cccc6c5sc5ccccc56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4cccc5c4sc4ccccc45)cc3)c3sc4ccccc4c23)cc1 ; c1ccc(-c2ccc(N(c3ccc(-c4cccc5c4oc4ccccc45)cc3)c3ccc(-c4cccc5c4sc4ccccc45)cc3)c3sc4ccccc4c23)cc1]

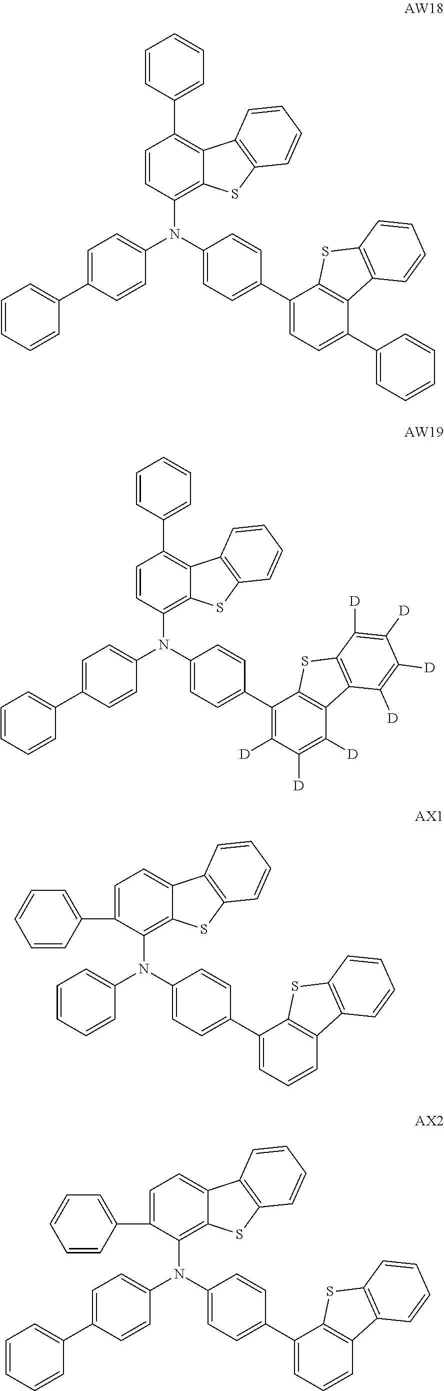 [SMILES: c1ccc(-c2ccc(N(c3ccc(-c4ccc(-c5ccccc5)c5c4sc4ccccc45)cc3)c3ccc(-c4ccccc4)c4c3sc3ccccc34)cc2)cc1 ; [2H]c1c([2H])c([2H])c2c(sc3c(-c4ccc(N(c5ccc(-c6ccccc6)cc5)c5ccc(-c6ccccc6)c6c5sc5ccccc56)cc4)c([2H])c([2H])c([2H])c32)c1[2H] ; c1ccc(-c2ccc3c(sc4ccccc43)c2N(c2ccccc2)c2ccc(-c3cccc4c3sc3ccccc34)cc2)cc1 ; c1ccc(-c2ccc(N(c3ccc(-c4cccc5c4sc4ccccc45)cc3)c3c(-c4ccccc4)ccc4c3sc3ccccc34)cc2)cc1]

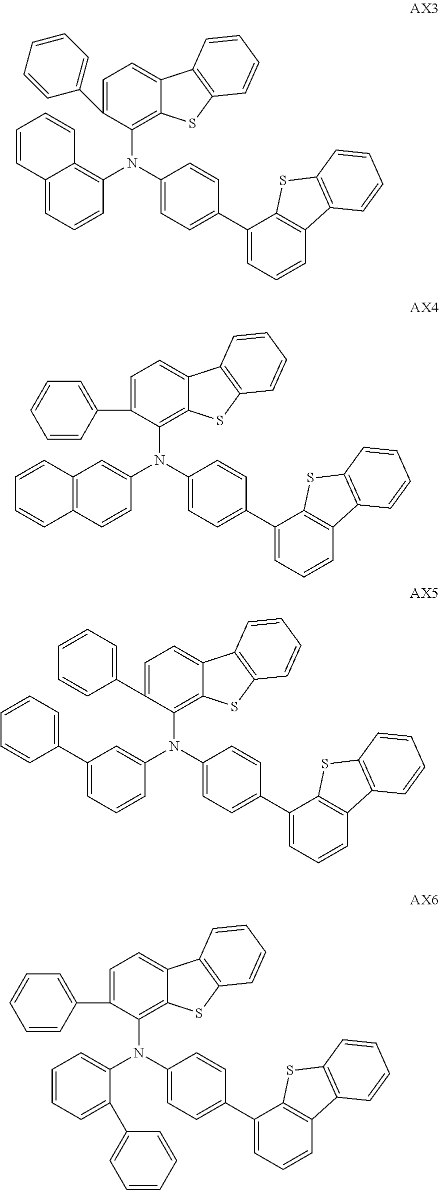 [SMILES: c1ccc(-c2ccc3c(sc4ccccc43)c2N(c2ccc(-c3cccc4c3sc3ccccc34)cc2)c2cccc3ccccc23)cc1 ; c1ccc(-c2ccc3c(sc4ccccc43)c2N(c2ccc(-c3cccc4c3sc3ccccc34)cc2)c2ccc3ccccc3c2)cc1 ; c1ccc(-c2cccc(N(c3ccc(-c4cccc5c4sc4ccccc45)cc3)c3c(-c4ccccc4)ccc4c3sc3ccccc34)c2)cc1 ; c1ccc(-c2ccccc2N(c2ccc(-c3cccc4c3sc3ccccc34)cc2)c2c(-c3ccccc3)ccc3c2sc2ccccc23)cc1]

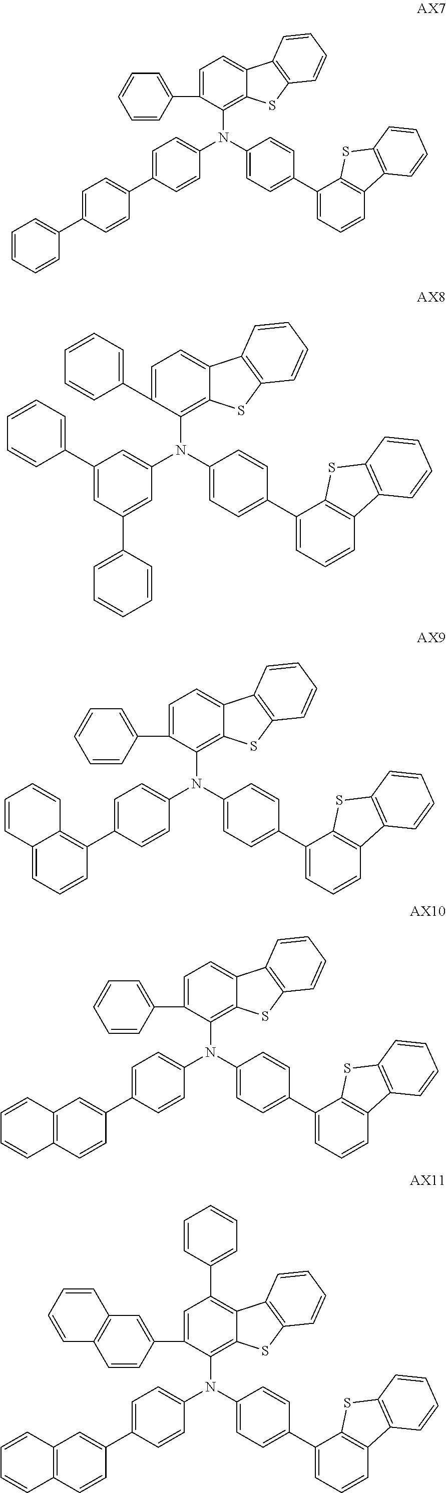 [SMILES: c1ccc(-c2ccc(-c3ccc(N(c4ccc(-c5cccc6c5sc5ccccc56)cc4)c4c(-c5ccccc5)ccc5c4sc4ccccc45)cc3)cc2)cc1 ; c1ccc(-c2cc(-c3ccccc3)cc(N(c3ccc(-c4cccc5c4sc4ccccc45)cc3)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2ccc(-c3cccc4c3sc3ccccc34)cc2)cc1 ; c1ccc(-c2ccc3c(sc4ccccc43)c2N(c2ccc(-c3ccc4ccccc4c3)cc2)c2ccc(-c3cccc4c3sc3ccccc34)cc2)cc1 ; c1ccc(-c2cc(-c3ccc4ccccc4c3)c(N(c3ccc(-c4ccc5ccccc5c4)cc3)c3ccc(-c4cccc5c4sc4ccccc45)cc3)c3sc4ccccc4c23)cc1]

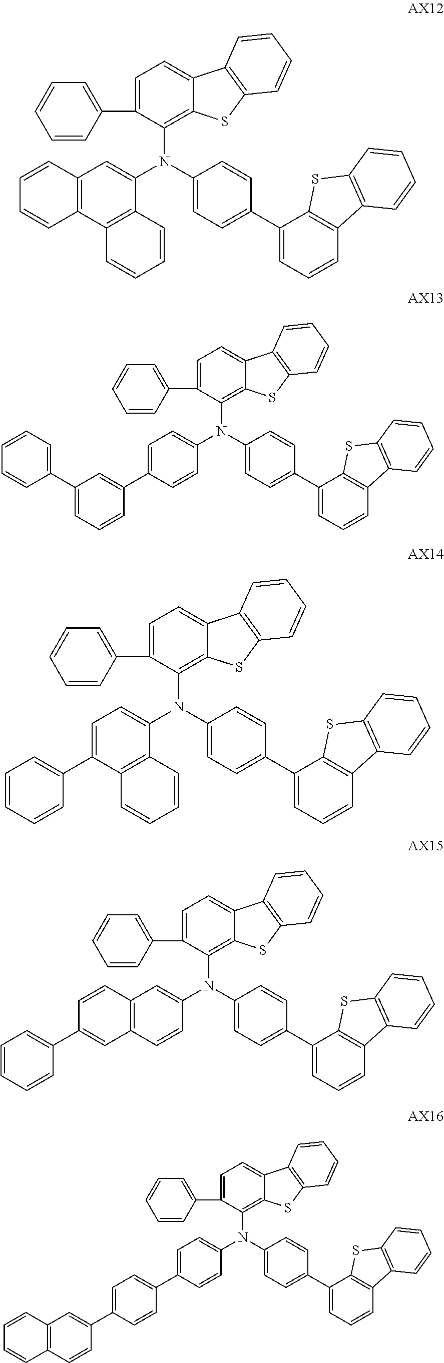 [SMILES: c1ccc(-c2ccc3c(sc4ccccc43)c2N(c2ccc(-c3cccc4c3sc3ccccc34)cc2)c2cc3ccccc3c3ccccc23)cc1 ; c1ccc(-c2cccc(-c3ccc(N(c4ccc(-c5cccc6c5sc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3cccc4c3sc3ccccc34)cc2)c2ccc(-c3ccccc3)c3ccccc23)cc1 ; c1ccc(-c2ccc3cc(N(c4ccc(-c5cccc6c5sc5ccccc56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3cccc4c3sc3ccccc34)cc2)cc1]

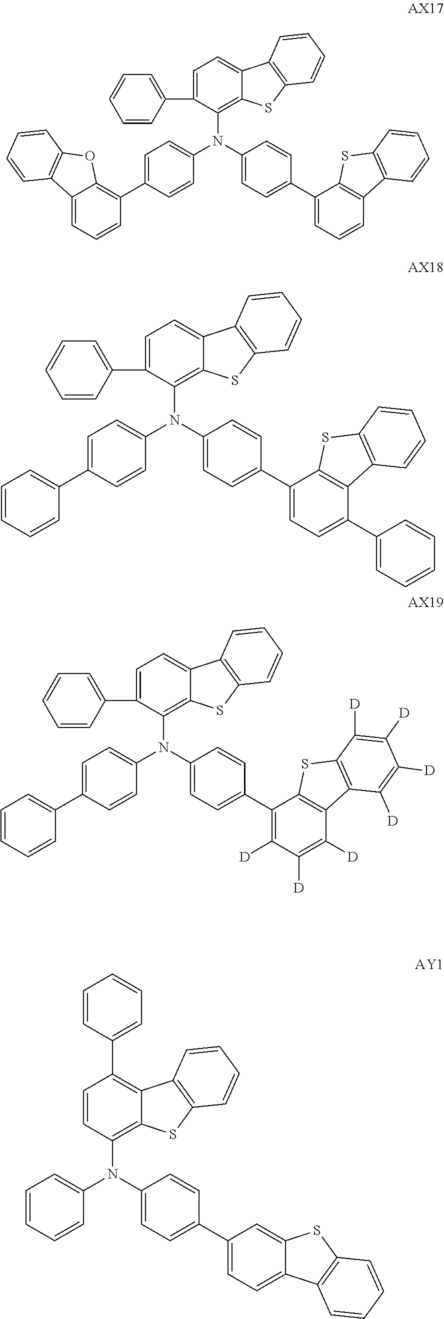 [SMILES: c1ccc(-c2ccc3c(sc4ccccc43)c2N(c2ccc(-c3cccc4c3oc3ccccc34)cc2)c2ccc(-c3cccc4c3sc3ccccc34)cc2)cc1 ; c1ccc(-c2ccc(N(c3ccc(-c4ccc(-c5ccccc5)c5c4sc4ccccc45)cc3)c3c(-c4ccccc4)ccc4c3sc3ccccc34)cc2)cc1 ; [2H]c1c([2H])c([2H])c2c(sc3c(-c4ccc(N(c5ccc(-c6ccccc6)cc5)c5c(-c6ccccc6)ccc6c5sc5ccccc56)cc4)c([2H])c([2H])c([2H])c32)c1[2H] ; c1ccc(-c2ccc(N(c3ccccc3)c3ccc(-c4ccc5c(c4)sc4ccccc45)cc3)c3sc4ccccc4c23)cc1]

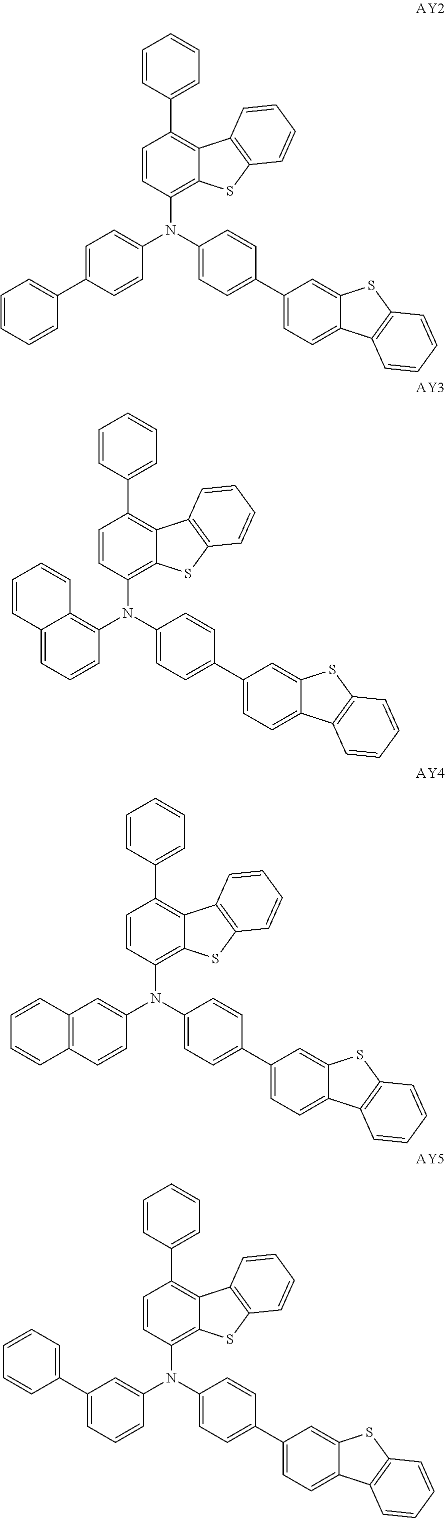 [SMILES: c1ccc(-c2ccc(N(c3ccc(-c4ccc5c(c4)sc4ccccc45)cc3)c3ccc(-c4ccccc4)c4c3sc3ccccc34)cc2)cc1 ; c1ccc(-c2ccc(N(c3ccc(-c4ccc5c(c4)sc4ccccc45)cc3)c3cccc4ccccc34)c3sc4ccccc4c23)cc1 ; c1ccc(-c2ccc(N(c3ccc(-c4ccc5c(c4)sc4ccccc45)cc3)c3ccc4ccccc4c3)c3sc4ccccc4c23)cc1 ; c1ccc(-c2cccc(N(c3ccc(-c4ccc5c(c4)sc4ccccc45)cc3)c3ccc(-c4ccccc4)c4c3sc3ccccc34)c2)cc1]

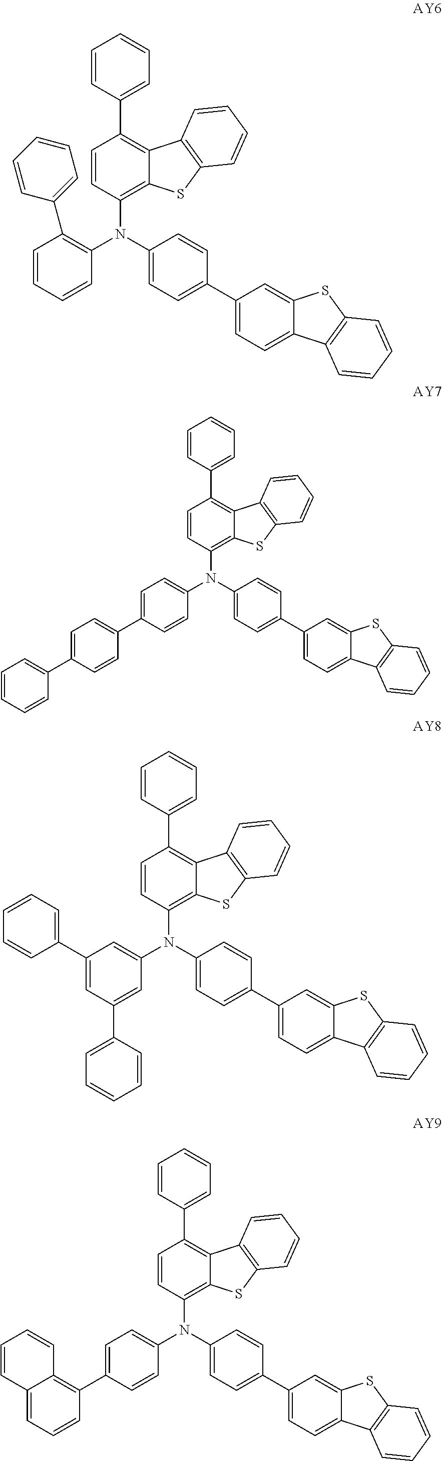 [SMILES: c1ccc(-c2ccccc2N(c2ccc(-c3ccc4c(c3)sc3ccccc34)cc2)c2ccc(-c3ccccc3)c3c2sc2ccccc23)cc1 ; c1ccc(-c2ccc(-c3ccc(N(c4ccc(-c5ccc6c(c5)sc5ccccc56)cc4)c4ccc(-c5ccccc5)c5c4sc4ccccc45)cc3)cc2)cc1 ; c1ccc(-c2cc(-c3ccccc3)cc(N(c3ccc(-c4ccc5c(c4)sc4ccccc45)cc3)c3ccc(-c4ccccc4)c4c3sc3ccccc34)c2)cc1 ; c1ccc(-c2ccc(N(c3ccc(-c4ccc5c(c4)sc4ccccc45)cc3)c3ccc(-c4cccc5ccccc45)cc3)c3sc4ccccc4c23)cc1]

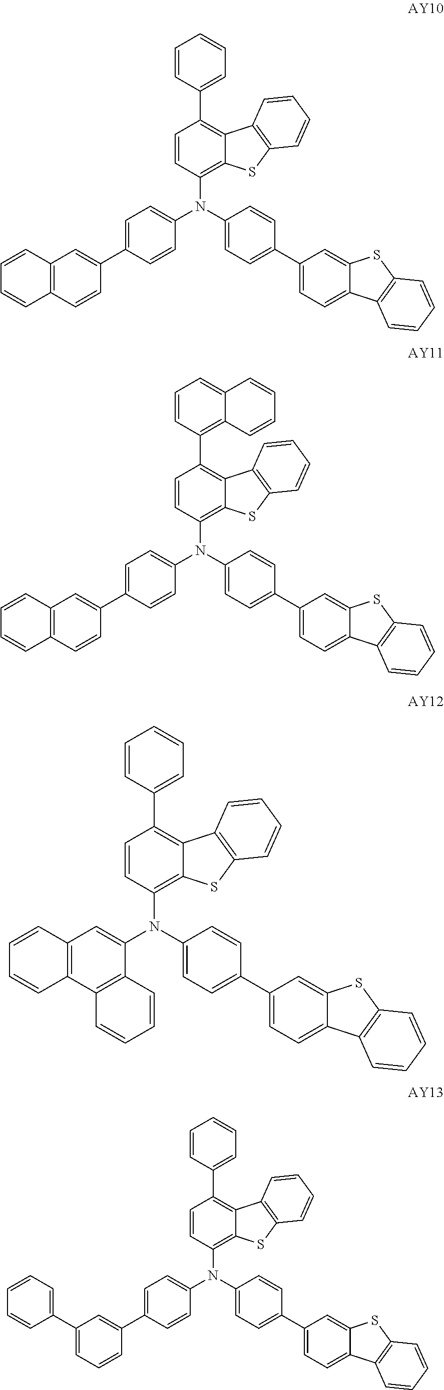 [SMILES: c1ccc(-c2ccc(N(c3ccc(-c4ccc5ccccc5c4)cc3)c3ccc(-c4ccc5c(c4)sc4ccccc45)cc3)c3sc4ccccc4c23)cc1 ; c1ccc2cc(-c3ccc(N(c4ccc(-c5ccc6c(c5)sc5ccccc56)cc4)c4ccc(-c5cccc6ccccc56)c5c4sc4ccccc45)cc3)ccc2c1 ; c1ccc(-c2ccc(N(c3ccc(-c4ccc5c(c4)sc4ccccc45)cc3)c3cc4ccccc4c4ccccc34)c3sc4ccccc4c23)cc1 ; c1ccc(-c2cccc(-c3ccc(N(c4ccc(-c5ccc6c(c5)sc5ccccc56)cc4)c4ccc(-c5ccccc5)c5c4sc4ccccc45)cc3)c2)cc1]

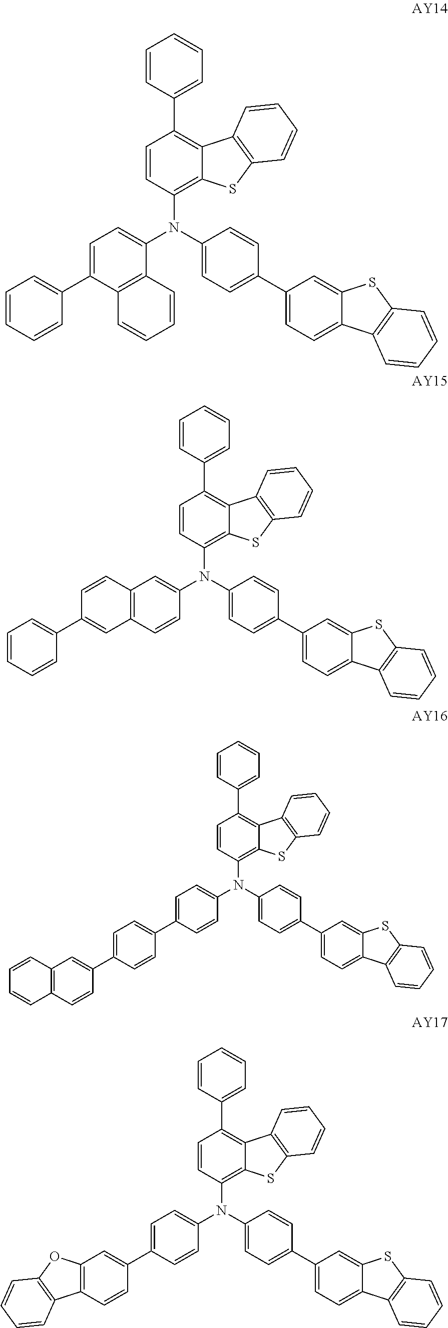 [SMILES: c1ccc(-c2ccc(N(c3ccc(-c4ccc5c(c4)sc4ccccc45)cc3)c3ccc(-c4ccccc4)c4c3sc3ccccc34)c3ccccc23)cc1 ; c1ccc(-c2ccc3cc(N(c4ccc(-c5ccc6c(c5)sc5ccccc56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4ccc5c(c4)sc4ccccc45)cc3)c3sc4ccccc4c23)cc1 ; c1ccc(-c2ccc(N(c3ccc(-c4ccc5c(c4)oc4ccccc45)cc3)c3ccc(-c4ccc5c(c4)sc4ccccc45)cc3)c3sc4ccccc4c23)cc1]

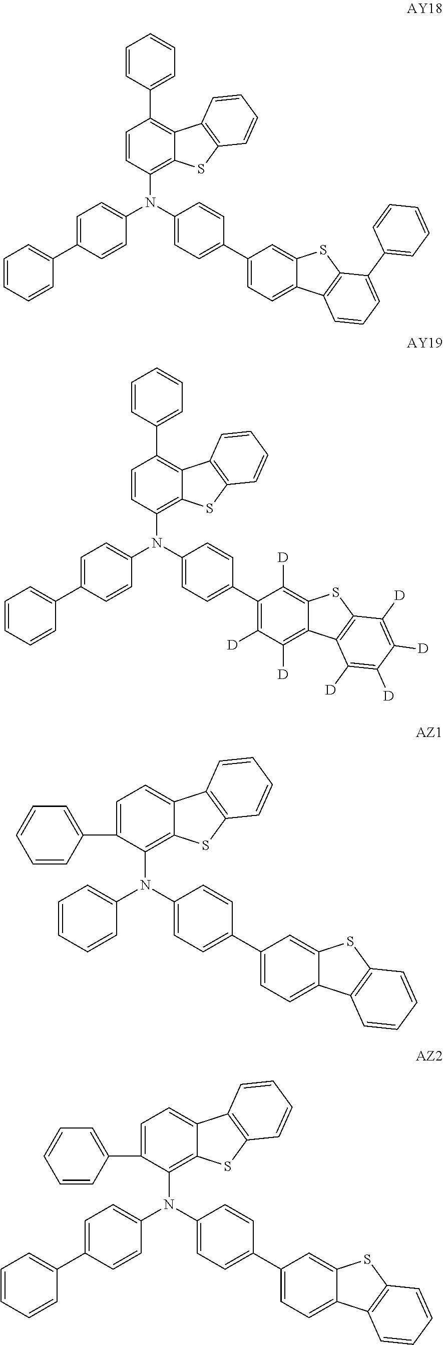 [SMILES: c1ccc(-c2ccc(N(c3ccc(-c4ccc5c(c4)sc4c(-c6ccccc6)cccc45)cc3)c3ccc(-c4ccccc4)c4c3sc3ccccc34)cc2)cc1 ; [2H]c1c([2H])c([2H])c2c(sc3c([2H])c(-c4ccc(N(c5ccc(-c6ccccc6)cc5)c5ccc(-c6ccccc6)c6c5sc5ccccc56)cc4)c([2H])c([2H])c32)c1[2H] ; c1ccc(-c2ccc3c(sc4ccccc43)c2N(c2ccccc2)c2ccc(-c3ccc4c(c3)sc3ccccc34)cc2)cc1 ; c1ccc(-c2ccc(N(c3ccc(-c4ccc5c(c4)sc4ccccc45)cc3)c3c(-c4ccccc4)ccc4c3sc3ccccc34)cc2)cc1]

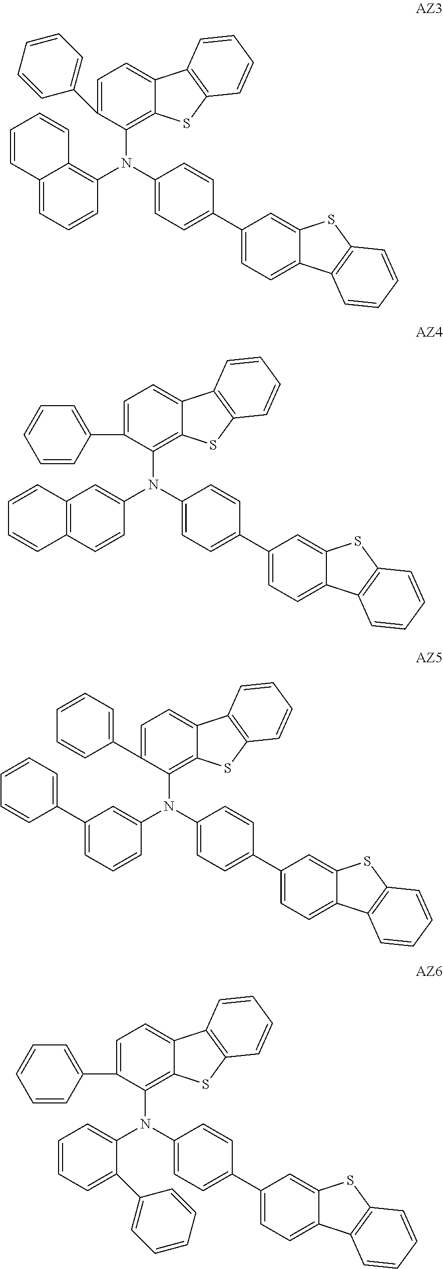 [SMILES: c1ccc(-c2ccc3c(sc4ccccc43)c2N(c2ccc(-c3ccc4c(c3)sc3ccccc34)cc2)c2cccc3ccccc23)cc1 ; c1ccc(-c2ccc3c(sc4ccccc43)c2N(c2ccc(-c3ccc4c(c3)sc3ccccc34)cc2)c2ccc3ccccc3c2)cc1 ; c1ccc(-c2cccc(N(c3ccc(-c4ccc5c(c4)sc4ccccc45)cc3)c3c(-c4ccccc4)ccc4c3sc3ccccc34)c2)cc1 ; c1ccc(-c2ccccc2N(c2ccc(-c3ccc4c(c3)sc3ccccc34)cc2)c2c(-c3ccccc3)ccc3c2sc2ccccc23)cc1]

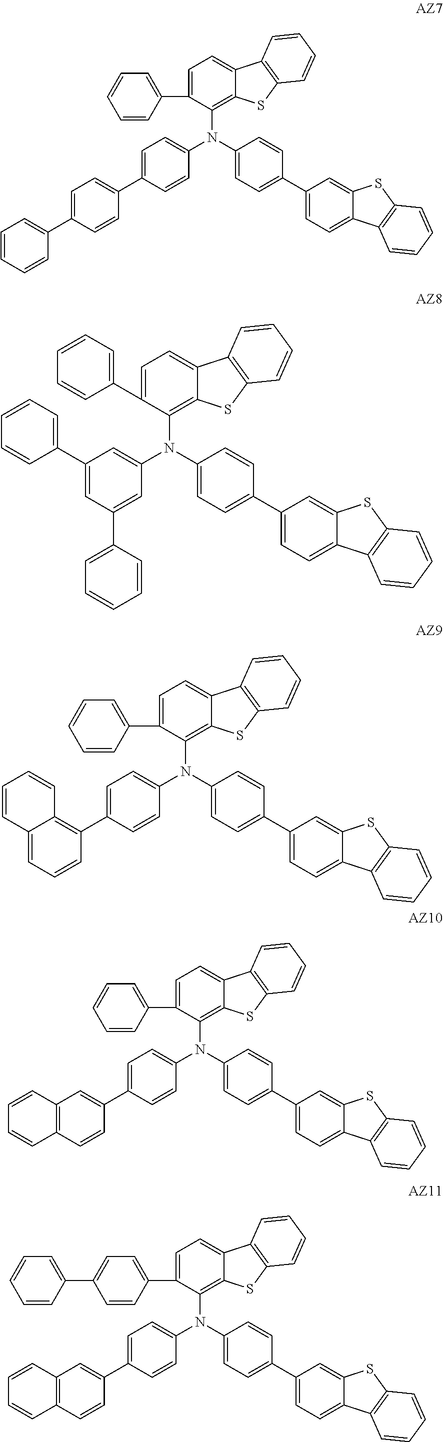 [SMILES: c1ccc(-c2ccc(-c3ccc(N(c4ccc(-c5ccc6c(c5)sc5ccccc56)cc4)c4c(-c5ccccc5)ccc5c4sc4ccccc45)cc3)cc2)cc1 ; c1ccc(-c2cc(-c3ccccc3)cc(N(c3ccc(-c4ccc5c(c4)sc4ccccc45)cc3)c3c(-c4ccccc4)ccc4c3sc3ccccc34)c2)cc1 ; c1ccc(-c2ccc3c(sc4ccccc43)c2N(c2ccc(-c3ccc4c(c3)sc3ccccc34)cc2)c2ccc(-c3cccc4ccccc34)cc2)cc1 ; c1ccc(-c2ccc3c(sc4ccccc43)c2N(c2ccc(-c3ccc4ccccc4c3)cc2)c2ccc(-c3ccc4c(c3)sc3ccccc34)cc2)cc1 ; c1ccc(-c2ccc(-c3ccc4c(sc5ccccc54)c3N(c3ccc(-c4ccc5ccccc5c4)cc3)c3ccc(-c4ccc5c(c4)sc4ccccc45)cc3)cc2)cc1]

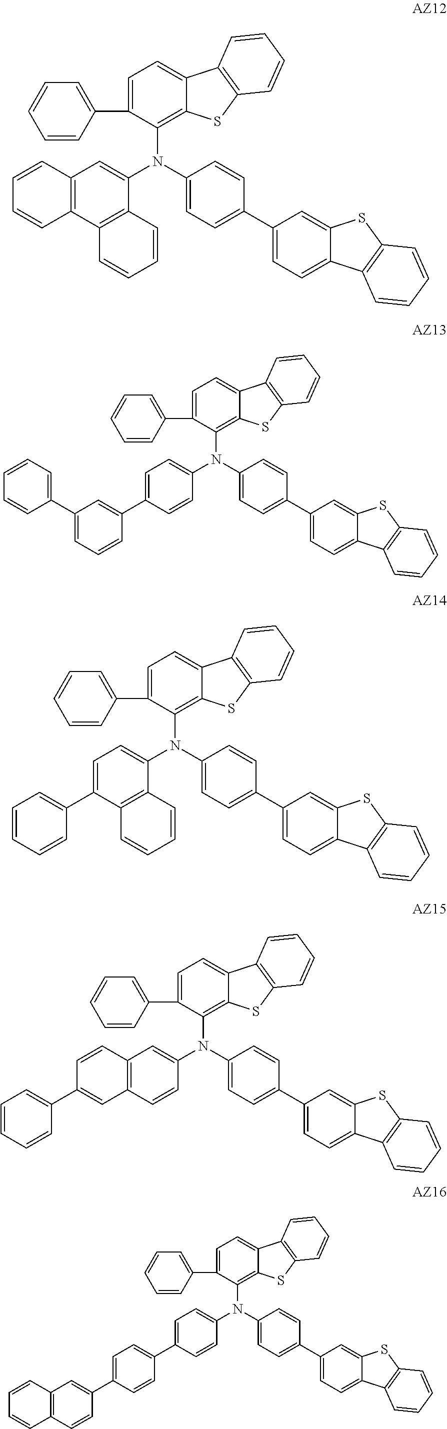 [SMILES: c1ccc(-c2ccc3c(sc4ccccc43)c2N(c2ccc(-c3ccc4c(c3)sc3ccccc34)cc2)c2cc3ccccc3c3ccccc23)cc1 ; c1ccc(-c2cccc(-c3ccc(N(c4ccc(-c5ccc6c(c5)sc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3ccc4c(c3)sc3ccccc34)cc2)c2ccc(-c3ccccc3)c3ccccc23)cc1 ; c1ccc(-c2ccc3cc(N(c4ccc(-c5ccc6c(c5)sc5ccccc56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3ccc4c(c3)sc3ccccc34)cc2)cc1]

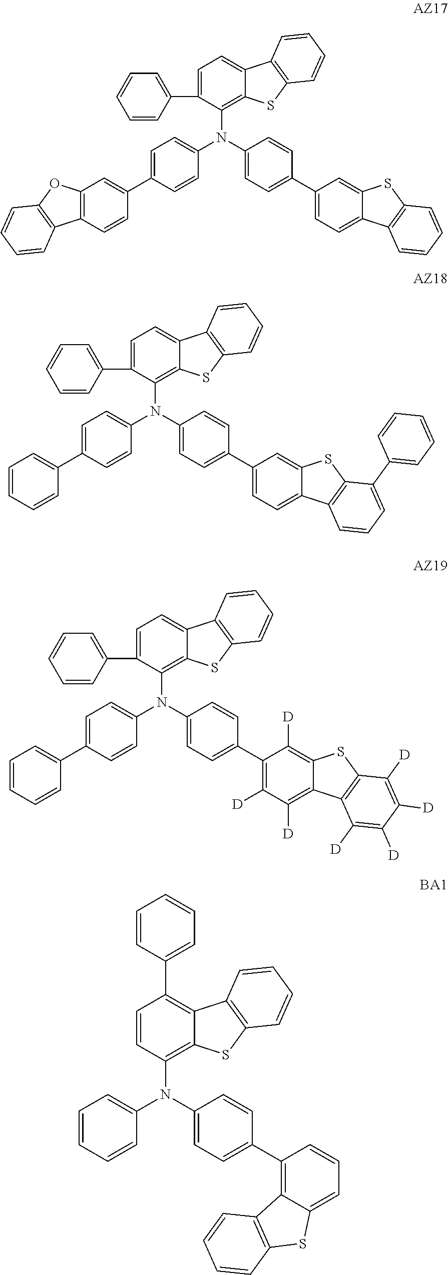 [SMILES: c1ccc(-c2ccc3c(sc4ccccc43)c2N(c2ccc(-c3ccc4c(c3)oc3ccccc34)cc2)c2ccc(-c3ccc4c(c3)sc3ccccc34)cc2)cc1 ; c1ccc(-c2ccc(N(c3ccc(-c4ccc5c(c4)sc4c(-c6ccccc6)cccc45)cc3)c3c(-c4ccccc4)ccc4c3sc3ccccc34)cc2)cc1 ; [2H]c1c([2H])c([2H])c2c(sc3c([2H])c(-c4ccc(N(c5ccc(-c6ccccc6)cc5)c5c(-c6ccccc6)ccc6c5sc5ccccc56)cc4)c([2H])c([2H])c32)c1[2H] ; c1ccc(-c2ccc(N(c3ccccc3)c3ccc(-c4cccc5sc6ccccc6c45)cc3)c3sc4ccccc4c23)cc1]

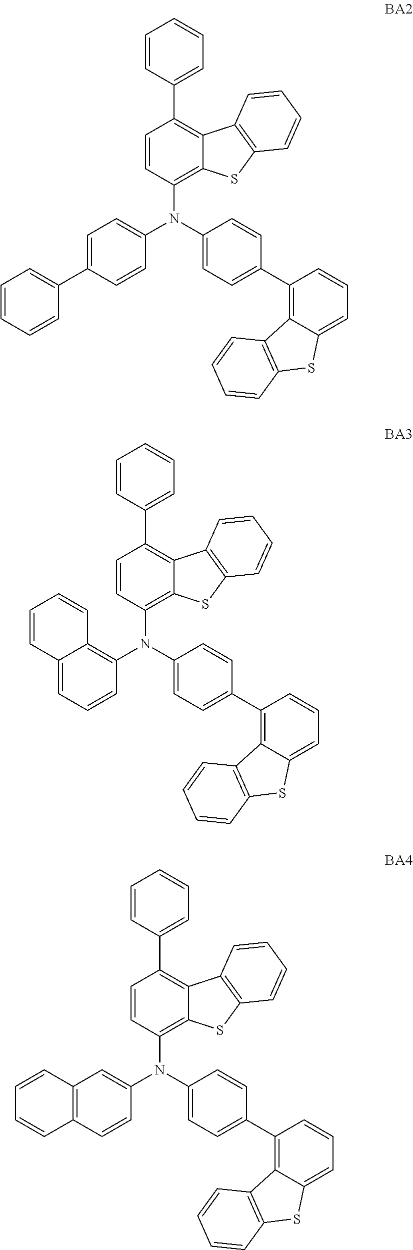 [SMILES: c1ccc(-c2ccc(N(c3ccc(-c4cccc5sc6ccccc6c45)cc3)c3ccc(-c4ccccc4)c4c3sc3ccccc34)cc2)cc1 ; c1ccc(-c2ccc(N(c3ccc(-c4cccc5sc6ccccc6c45)cc3)c3cccc4ccccc34)c3sc4ccccc4c23)cc1 ; c1ccc(-c2ccc(N(c3ccc(-c4cccc5sc6ccccc6c45)cc3)c3ccc4ccccc4c3)c3sc4ccccc4c23)cc1]

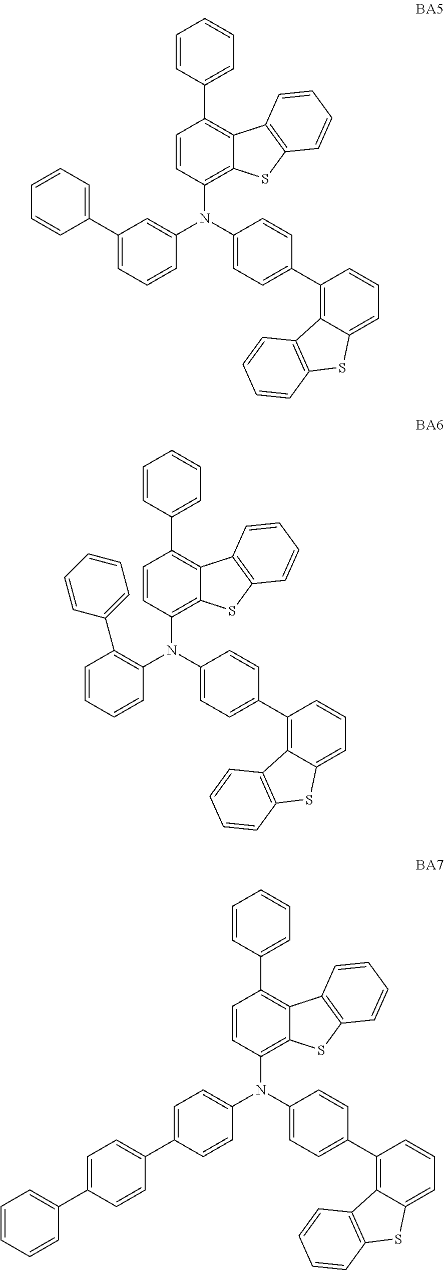 [SMILES: c1ccc(-c2cccc(N(c3ccc(-c4cccc5sc6ccccc6c45)cc3)c3ccc(-c4ccccc4)c4c3sc3ccccc34)c2)cc1 ; c1ccc(-c2ccccc2N(c2ccc(-c3cccc4sc5ccccc5c34)cc2)c2ccc(-c3ccccc3)c3c2sc2ccccc23)cc1 ; c1ccc(-c2ccc(-c3ccc(N(c4ccc(-c5cccc6sc7ccccc7c56)cc4)c4ccc(-c5ccccc5)c5c4sc4ccccc45)cc3)cc2)cc1]

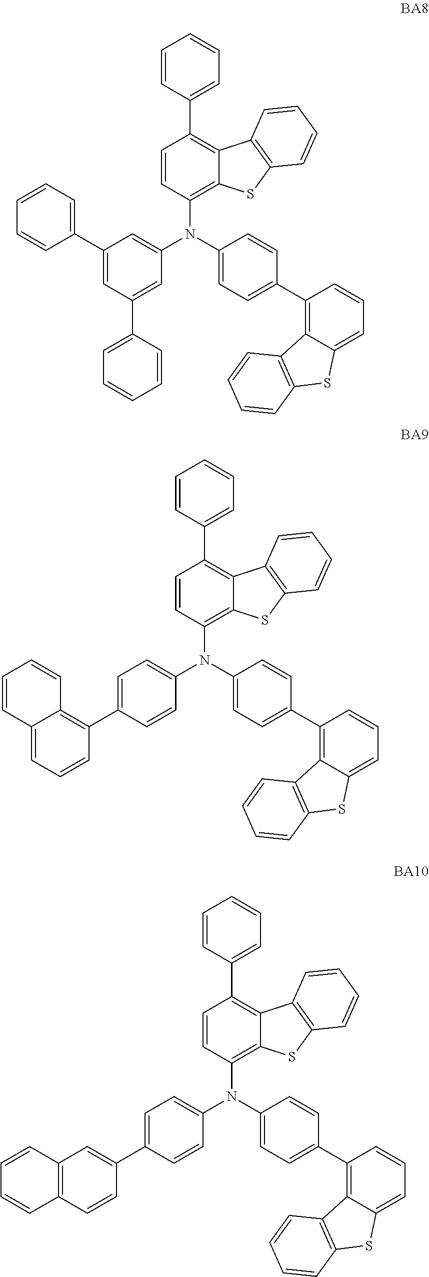 [SMILES: c1ccc(-c2cc(-c3ccccc3)cc(N(c3ccc(-c4cccc5sc6ccccc6c45)cc3)c3ccc(-c4ccccc4)c4c3sc3ccccc34)c2)cc1 ; c1ccc(-c2ccc(N(c3ccc(-c4cccc5ccccc45)cc3)c3ccc(-c4cccc5sc6ccccc6c45)cc3)c3sc4ccccc4c23)cc1 ; c1ccc(-c2ccc(N(c3ccc(-c4ccc5ccccc5c4)cc3)c3ccc(-c4cccc5sc6ccccc6c45)cc3)c3sc4ccccc4c23)cc1]

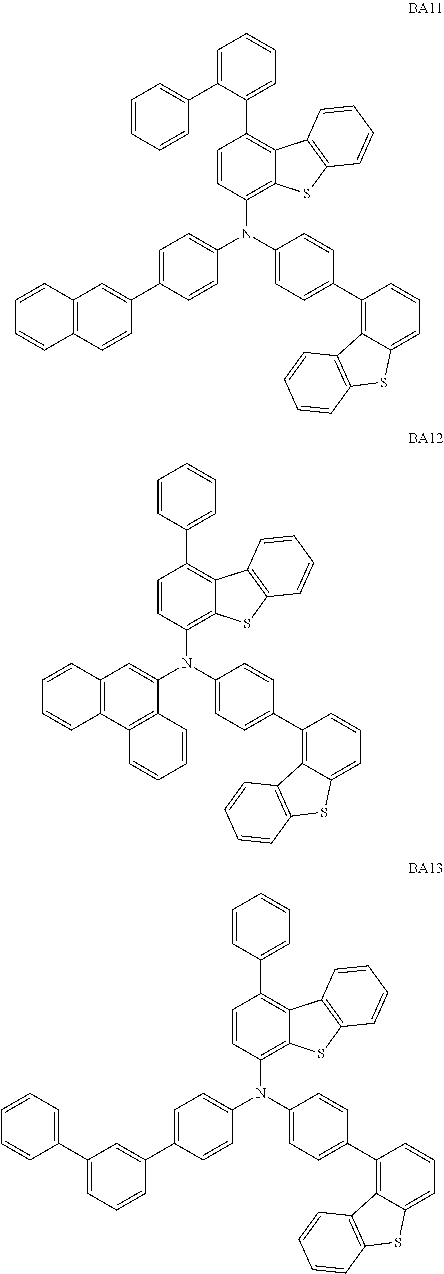 [SMILES: c1ccc(-c2ccccc2-c2ccc(N(c3ccc(-c4ccc5ccccc5c4)cc3)c3ccc(-c4cccc5sc6ccccc6c45)cc3)c3sc4ccccc4c23)cc1 ; c1ccc(-c2ccc(N(c3ccc(-c4cccc5sc6ccccc6c45)cc3)c3cc4ccccc4c4ccccc34)c3sc4ccccc4c23)cc1 ; c1ccc(-c2cccc(-c3ccc(N(c4ccc(-c5cccc6sc7ccccc7c56)cc4)c4ccc(-c5ccccc5)c5c4sc4ccccc45)cc3)c2)cc1]

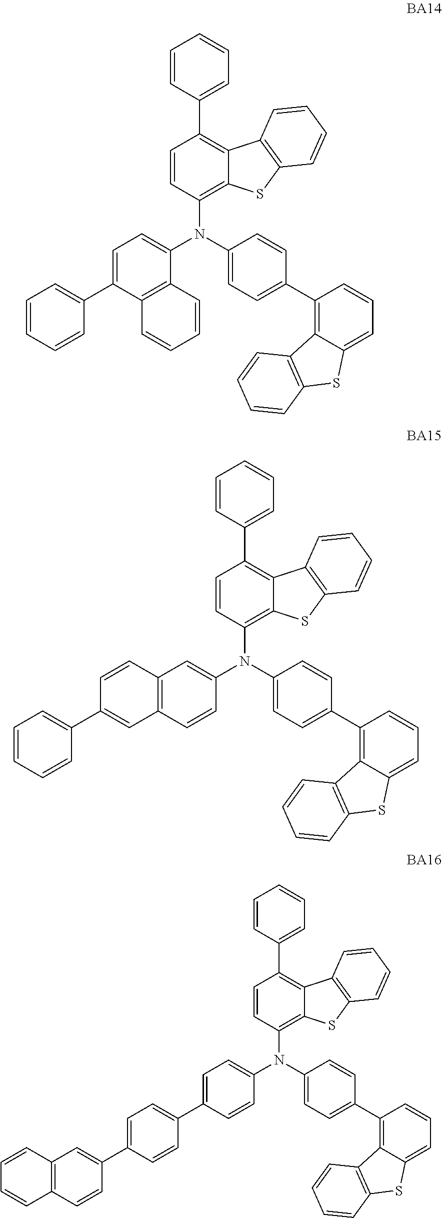 [SMILES: c1ccc(-c2ccc(N(c3ccc(-c4cccc5sc6ccccc6c45)cc3)c3ccc(-c4ccccc4)c4c3sc3ccccc34)c3ccccc23)cc1 ; c1ccc(-c2ccc3cc(N(c4ccc(-c5cccc6sc7ccccc7c56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4cccc5sc6ccccc6c45)cc3)c3sc4ccccc4c23)cc1]

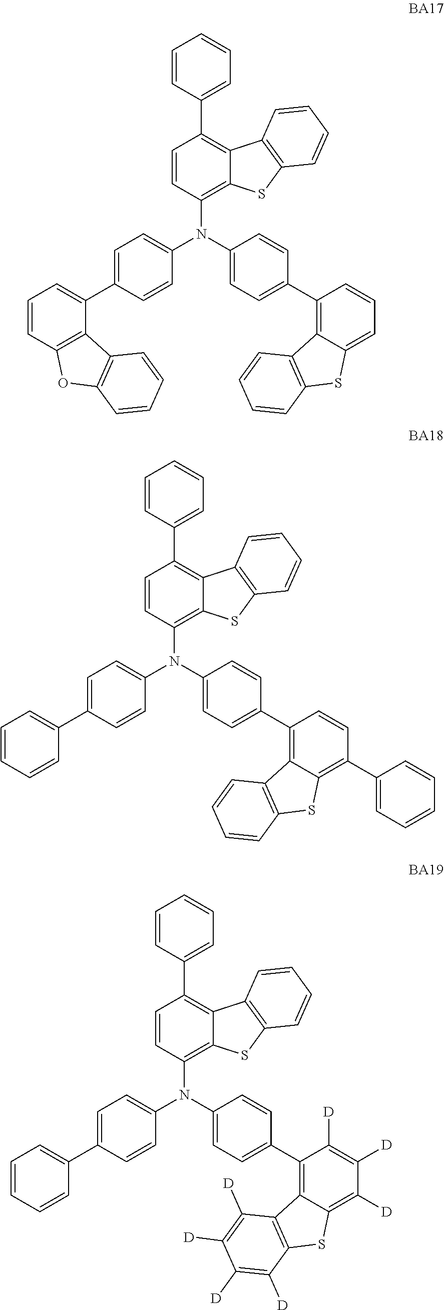 [SMILES: c1ccc(-c2ccc(N(c3ccc(-c4cccc5oc6ccccc6c45)cc3)c3ccc(-c4cccc5sc6ccccc6c45)cc3)c3sc4ccccc4c23)cc1 ; c1ccc(-c2ccc(N(c3ccc(-c4ccc(-c5ccccc5)c5sc6ccccc6c45)cc3)c3ccc(-c4ccccc4)c4c3sc3ccccc34)cc2)cc1 ; [2H]c1c([2H])c([2H])c2c(sc3c([2H])c([2H])c([2H])c(-c4ccc(N(c5ccc(-c6ccccc6)cc5)c5ccc(-c6ccccc6)c6c5sc5ccccc56)cc4)c32)c1[2H]]

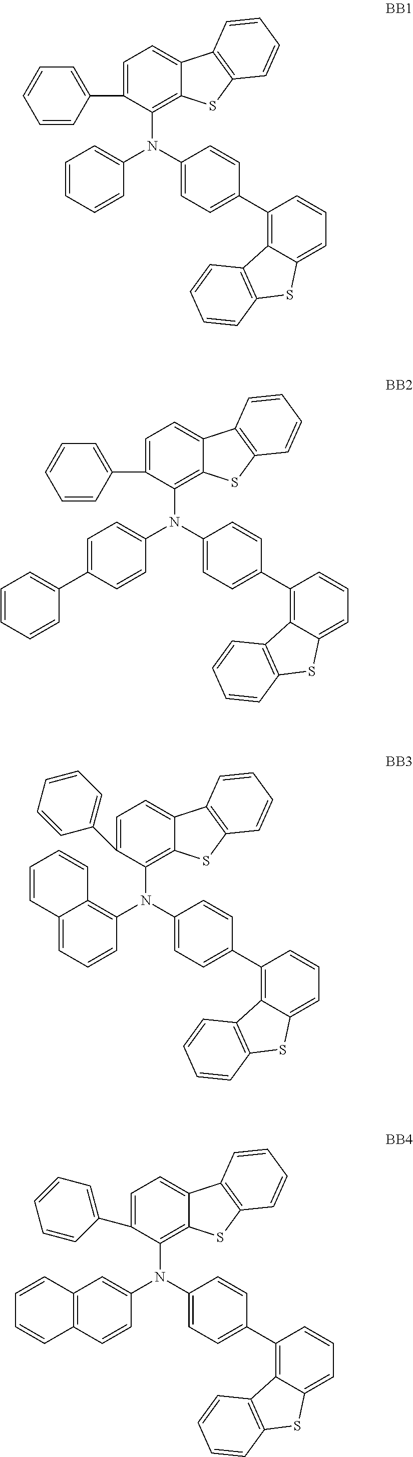 [SMILES: c1ccc(-c2ccc3c(sc4ccccc43)c2N(c2ccccc2)c2ccc(-c3cccc4sc5ccccc5c34)cc2)cc1 ; c1ccc(-c2ccc(N(c3ccc(-c4cccc5sc6ccccc6c45)cc3)c3c(-c4ccccc4)ccc4c3sc3ccccc34)cc2)cc1 ; c1ccc(-c2ccc3c(sc4ccccc43)c2N(c2ccc(-c3cccc4sc5ccccc5c34)cc2)c2cccc3ccccc23)cc1 ; c1ccc(-c2ccc3c(sc4ccccc43)c2N(c2ccc(-c3cccc4sc5ccccc5c34)cc2)c2ccc3ccccc3c2)cc1]

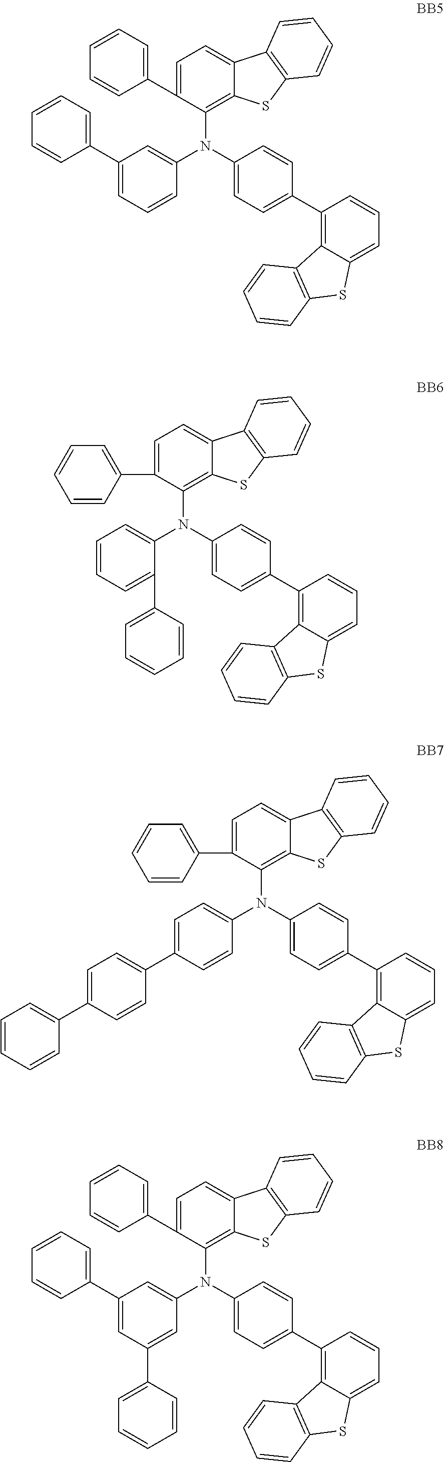 [SMILES: c1ccc(-c2cccc(N(c3ccc(-c4cccc5sc6ccccc6c45)cc3)c3c(-c4ccccc4)ccc4c3sc3ccccc34)c2)cc1 ; c1ccc(-c2ccccc2N(c2ccc(-c3cccc4sc5ccccc5c34)cc2)c2c(-c3ccccc3)ccc3c2sc2ccccc23)cc1 ; c1ccc(-c2ccc(-c3ccc(N(c4ccc(-c5cccc6sc7ccccc7c56)cc4)c4c(-c5ccccc5)ccc5c4sc4ccccc45)cc3)cc2)cc1 ; c1ccc(-c2cc(-c3ccccc3)cc(N(c3ccc(-c4cccc5sc6ccccc6c45)cc3)c3c(-c4ccccc4)ccc4c3sc3ccccc34)c2)cc1]

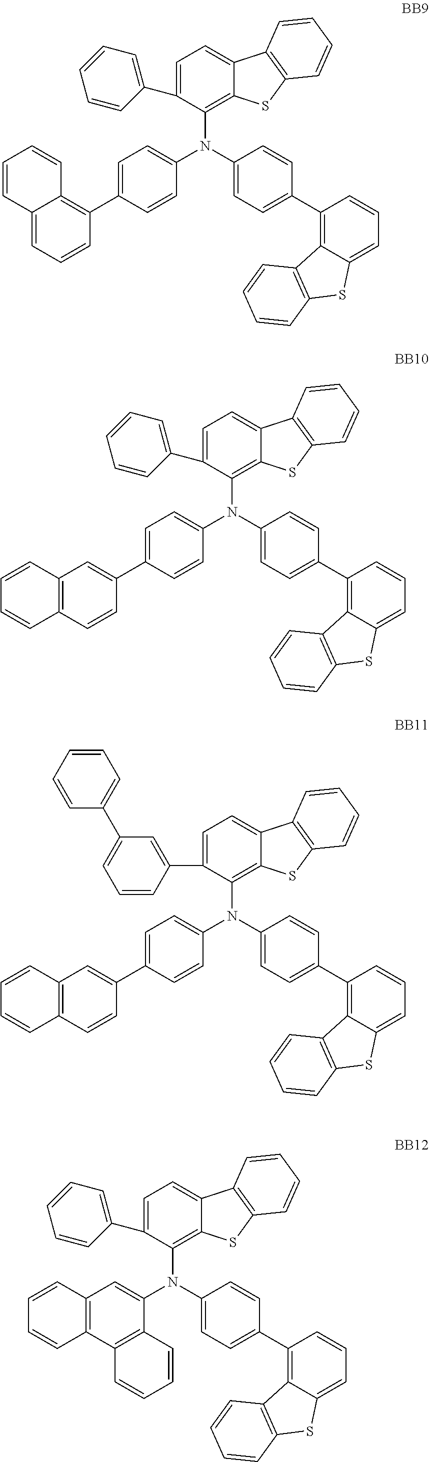 [SMILES: c1ccc(-c2ccc3c(sc4ccccc43)c2N(c2ccc(-c3cccc4ccccc34)cc2)c2ccc(-c3cccc4sc5ccccc5c34)cc2)cc1 ; c1ccc(-c2ccc3c(sc4ccccc43)c2N(c2ccc(-c3ccc4ccccc4c3)cc2)c2ccc(-c3cccc4sc5ccccc5c34)cc2)cc1 ; c1ccc(-c2cccc(-c3ccc4c(sc5ccccc54)c3N(c3ccc(-c4ccc5ccccc5c4)cc3)c3ccc(-c4cccc5sc6ccccc6c45)cc3)c2)cc1 ; c1ccc(-c2ccc3c(sc4ccccc43)c2N(c2ccc(-c3cccc4sc5ccccc5c34)cc2)c2cc3ccccc3c3ccccc23)cc1]

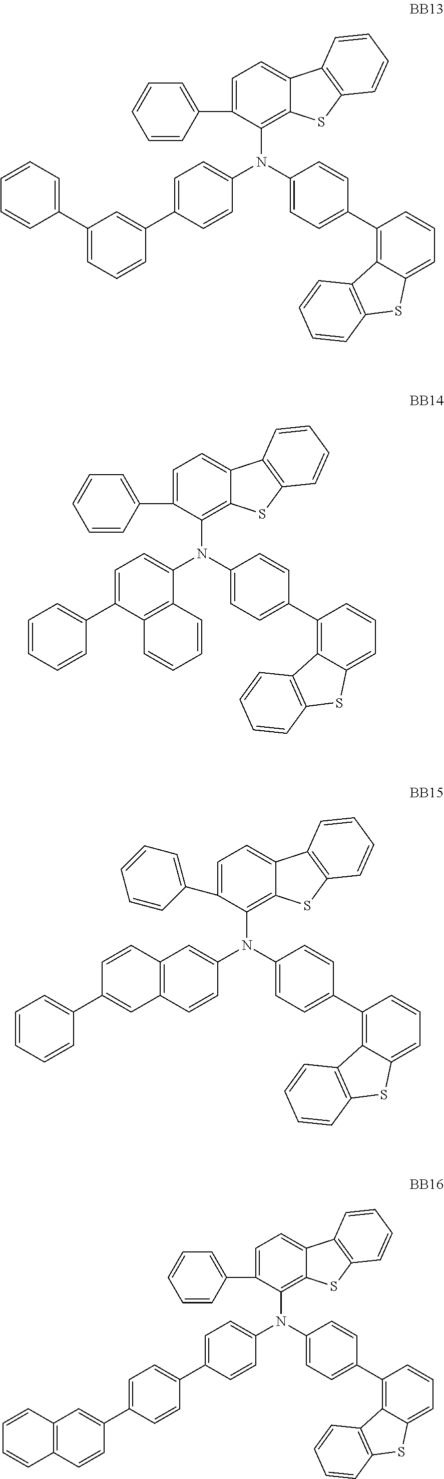 [SMILES: c1ccc(-c2cccc(-c3ccc(N(c4ccc(-c5cccc6sc7ccccc7c56)cc4)c4c(-c5ccccc5)ccc5c4sc4ccccc45)cc3)c2)cc1 ; c1ccc(-c2ccc3c(sc4ccccc43)c2N(c2ccc(-c3cccc4sc5ccccc5c34)cc2)c2ccc(-c3ccccc3)c3ccccc23)cc1 ; c1ccc(-c2ccc3cc(N(c4ccc(-c5cccc6sc7ccccc7c56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3cccc4sc5ccccc5c34)cc2)cc1]

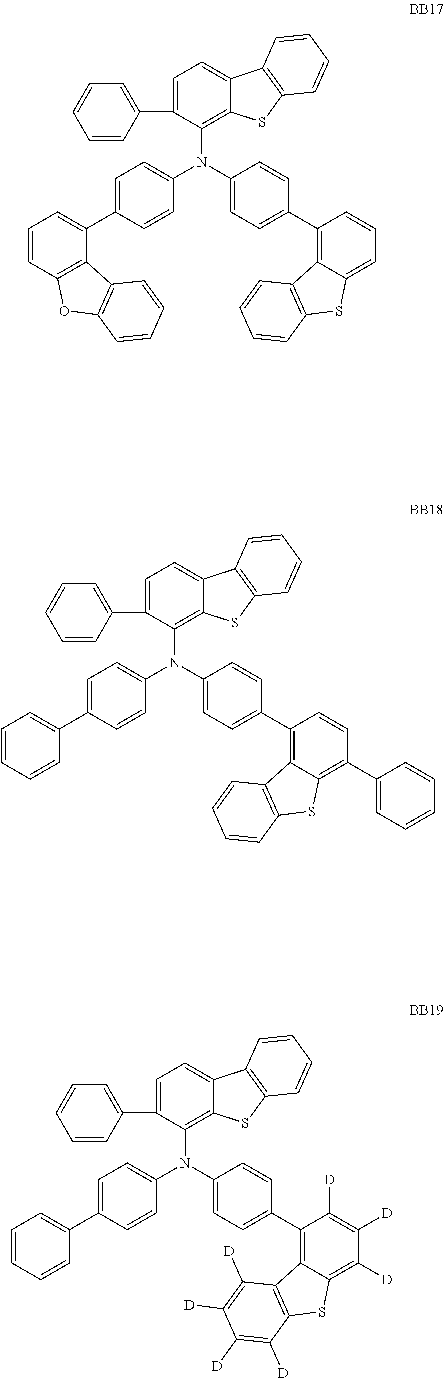 [SMILES: c1ccc(-c2ccc3c(sc4ccccc43)c2N(c2ccc(-c3cccc4oc5ccccc5c34)cc2)c2ccc(-c3cccc4sc5ccccc5c34)cc2)cc1 ; c1ccc(-c2ccc(N(c3ccc(-c4ccc(-c5ccccc5)c5sc6ccccc6c45)cc3)c3c(-c4ccccc4)ccc4c3sc3ccccc34)cc2)cc1 ; [2H]c1c([2H])c([2H])c2c(sc3c([2H])c([2H])c([2H])c(-c4ccc(N(c5ccc(-c6ccccc6)cc5)c5c(-c6ccccc6)ccc6c5sc5ccccc56)cc4)c32)c1[2H]]

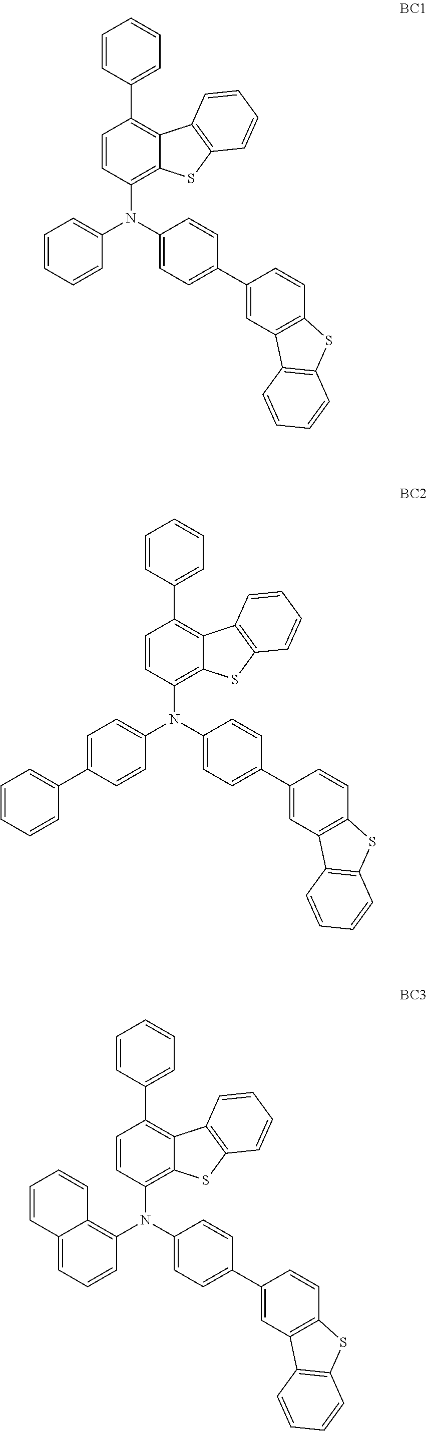 [SMILES: c1ccc(-c2ccc(N(c3ccccc3)c3ccc(-c4ccc5sc6ccccc6c5c4)cc3)c3sc4ccccc4c23)cc1 ; c1ccc(-c2ccc(N(c3ccc(-c4ccc5sc6ccccc6c5c4)cc3)c3ccc(-c4ccccc4)c4c3sc3ccccc34)cc2)cc1 ; c1ccc(-c2ccc(N(c3ccc(-c4ccc5sc6ccccc6c5c4)cc3)c3cccc4ccccc34)c3sc4ccccc4c23)cc1]

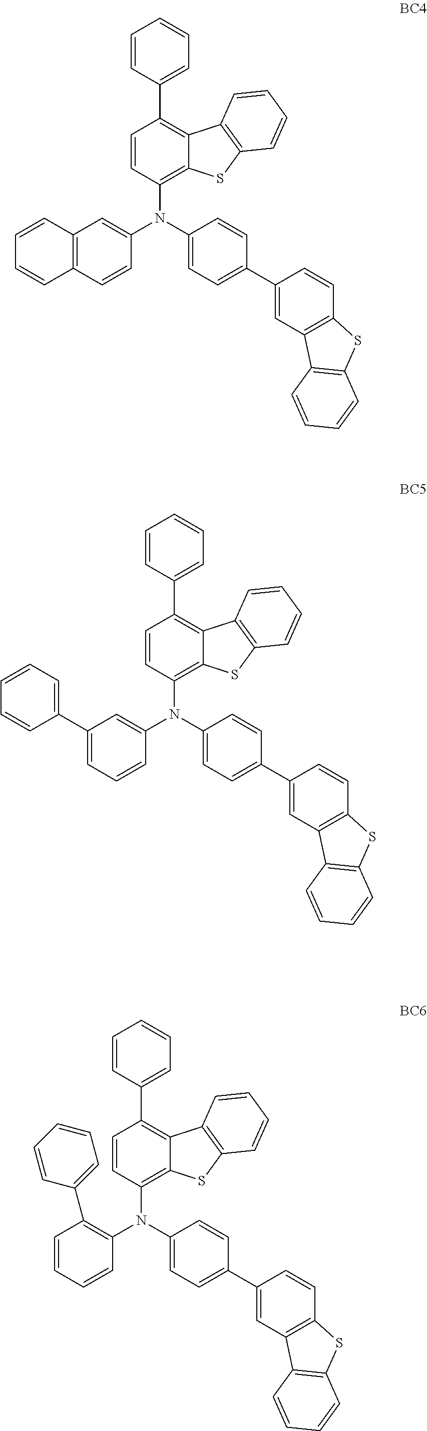 [SMILES: c1ccc(-c2ccc(N(c3ccc(-c4ccc5sc6ccccc6c5c4)cc3)c3ccc4ccccc4c3)c3sc4ccccc4c23)cc1 ; c1ccc(-c2cccc(N(c3ccc(-c4ccc5sc6ccccc6c5c4)cc3)c3ccc(-c4ccccc4)c4c3sc3ccccc34)c2)cc1 ; c1ccc(-c2ccccc2N(c2ccc(-c3ccc4sc5ccccc5c4c3)cc2)c2ccc(-c3ccccc3)c3c2sc2ccccc23)cc1]

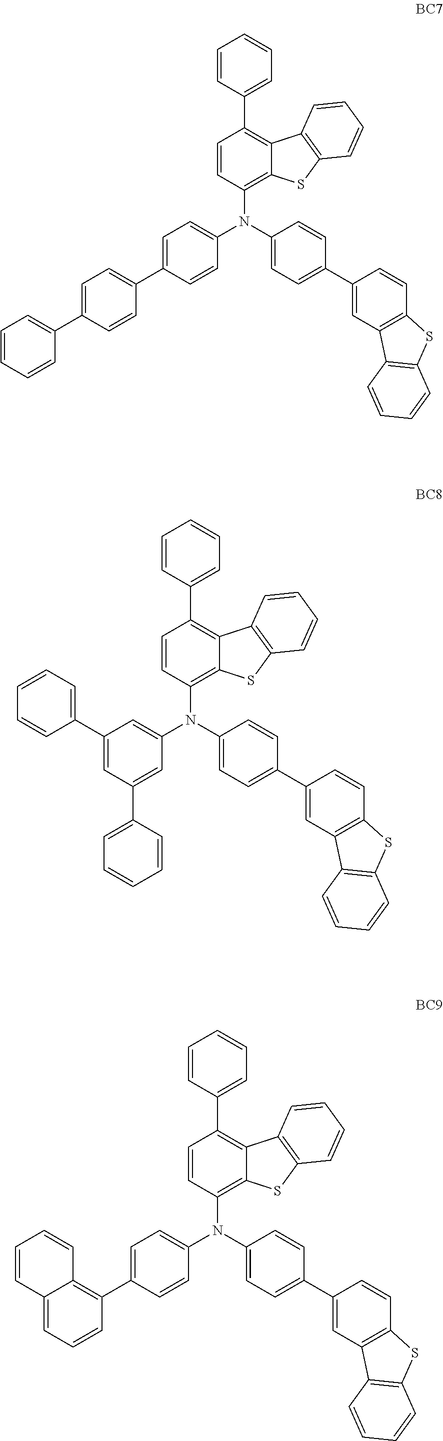 [SMILES: c1ccc(-c2ccc(-c3ccc(N(c4ccc(-c5ccc6sc7ccccc7c6c5)cc4)c4ccc(-c5ccccc5)c5c4sc4ccccc45)cc3)cc2)cc1 ; c1ccc(-c2cc(-c3ccccc3)cc(N(c3ccc(-c4ccc5sc6ccccc6c5c4)cc3)c3ccc(-c4ccccc4)c4c3sc3ccccc34)c2)cc1 ; c1ccc(-c2ccc(N(c3ccc(-c4ccc5sc6ccccc6c5c4)cc3)c3ccc(-c4cccc5ccccc45)cc3)c3sc4ccccc4c23)cc1]

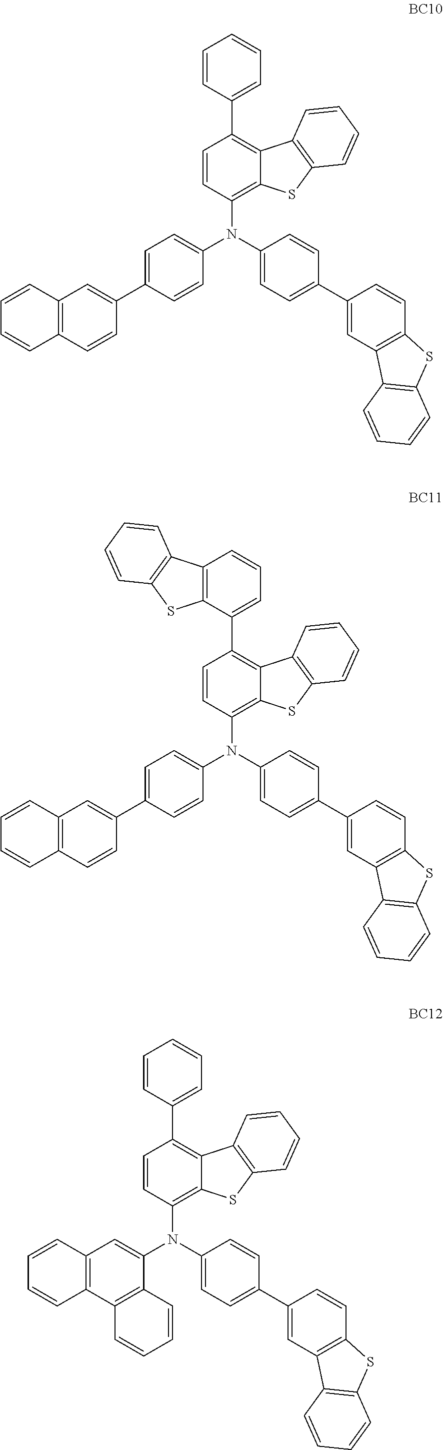 [SMILES: c1ccc(-c2ccc(N(c3ccc(-c4ccc5ccccc5c4)cc3)c3ccc(-c4ccc5sc6ccccc6c5c4)cc3)c3sc4ccccc4c23)cc1 ; c1ccc2cc(-c3ccc(N(c4ccc(-c5ccc6sc7ccccc7c6c5)cc4)c4ccc(-c5cccc6c5sc5ccccc56)c5c4sc4ccccc45)cc3)ccc2c1 ; c1ccc(-c2ccc(N(c3ccc(-c4ccc5sc6ccccc6c5c4)cc3)c3cc4ccccc4c4ccccc34)c3sc4ccccc4c23)cc1]

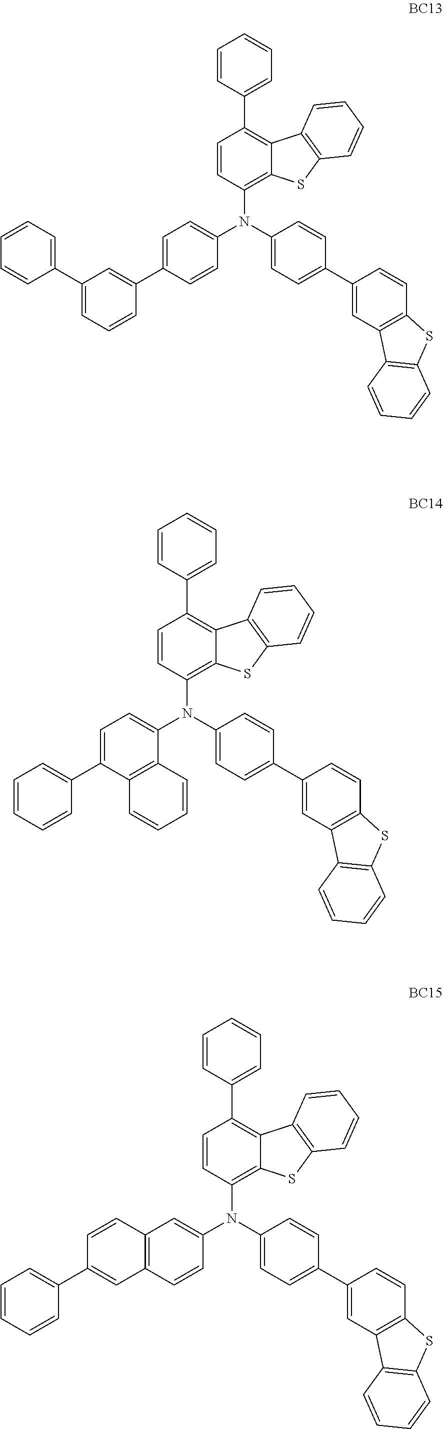 [SMILES: c1ccc(-c2cccc(-c3ccc(N(c4ccc(-c5ccc6sc7ccccc7c6c5)cc4)c4ccc(-c5ccccc5)c5c4sc4ccccc45)cc3)c2)cc1 ; c1ccc(-c2ccc(N(c3ccc(-c4ccc5sc6ccccc6c5c4)cc3)c3ccc(-c4ccccc4)c4c3sc3ccccc34)c3ccccc23)cc1 ; c1ccc(-c2ccc3cc(N(c4ccc(-c5ccc6sc7ccccc7c6c5)cc4)c4ccc(-c5ccccc5)c5c4sc4ccccc45)ccc3c2)cc1]

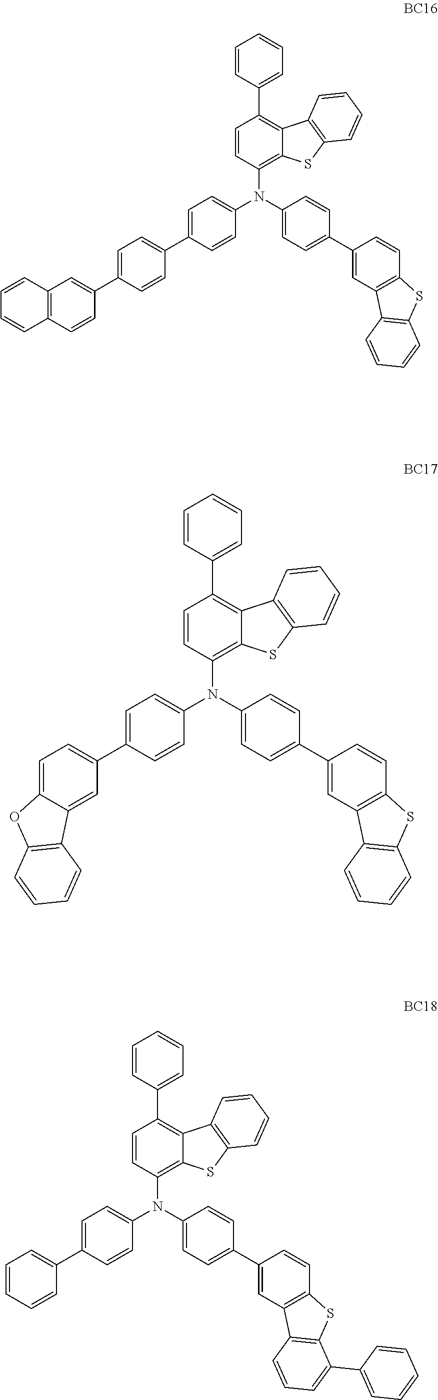 [SMILES: c1ccc(-c2ccc(N(c3ccc(-c4ccc(-c5ccc6ccccc6c5)cc4)cc3)c3ccc(-c4ccc5sc6ccccc6c5c4)cc3)c3sc4ccccc4c23)cc1 ; c1ccc(-c2ccc(N(c3ccc(-c4ccc5oc6ccccc6c5c4)cc3)c3ccc(-c4ccc5sc6ccccc6c5c4)cc3)c3sc4ccccc4c23)cc1 ; c1ccc(-c2ccc(N(c3ccc(-c4ccc5sc6c(-c7ccccc7)cccc6c5c4)cc3)c3ccc(-c4ccccc4)c4c3sc3ccccc34)cc2)cc1]

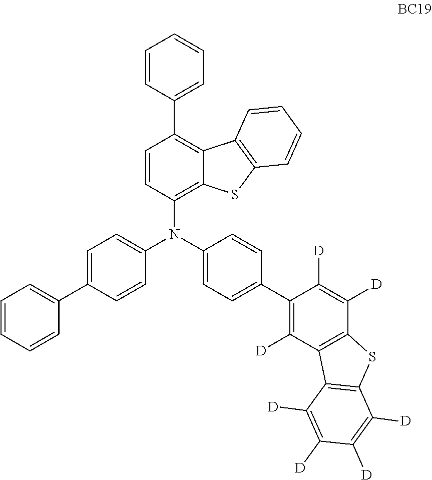 [SMILES: [2H]c1c([2H])c([2H])c2c(sc3c([2H])c([2H])c(-c4ccc(N(c5ccc(-c6ccccc6)cc5)c5ccc(-c6ccccc6)c6c5sc5ccccc56)cc4)c([2H])c32)c1[2H]]

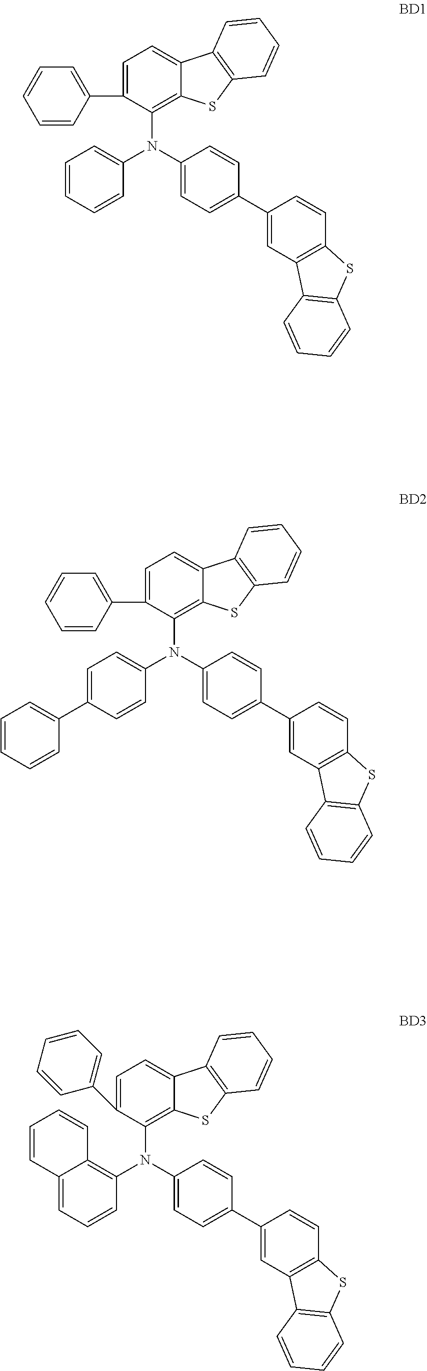 [SMILES: c1ccc(-c2ccc3c(sc4ccccc43)c2N(c2ccccc2)c2ccc(-c3ccc4sc5ccccc5c4c3)cc2)cc1 ; c1ccc(-c2ccc(N(c3ccc(-c4ccc5sc6ccccc6c5c4)cc3)c3c(-c4ccccc4)ccc4c3sc3ccccc34)cc2)cc1 ; c1ccc(-c2ccc3c(sc4ccccc43)c2N(c2ccc(-c3ccc4sc5ccccc5c4c3)cc2)c2cccc3ccccc23)cc1]

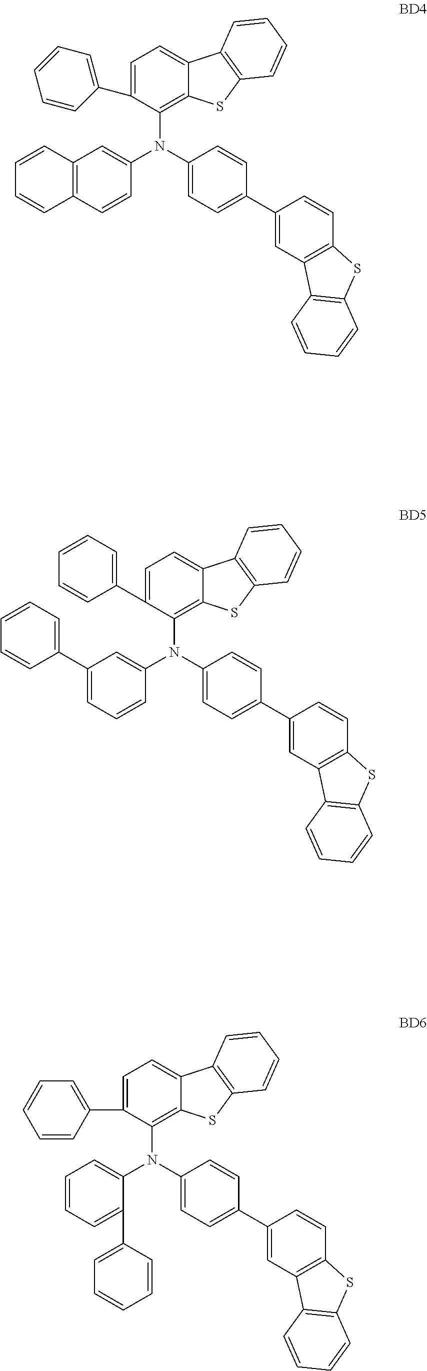 [SMILES: c1ccc(-c2ccc3c(sc4ccccc43)c2N(c2ccc(-c3ccc4sc5ccccc5c4c3)cc2)c2ccc3ccccc3c2)cc1 ; c1ccc(-c2cccc(N(c3ccc(-c4ccc5sc6ccccc6c5c4)cc3)c3c(-c4ccccc4)ccc4c3sc3ccccc34)c2)cc1 ; c1ccc(-c2ccccc2N(c2ccc(-c3ccc4sc5ccccc5c4c3)cc2)c2c(-c3ccccc3)ccc3c2sc2ccccc23)cc1]

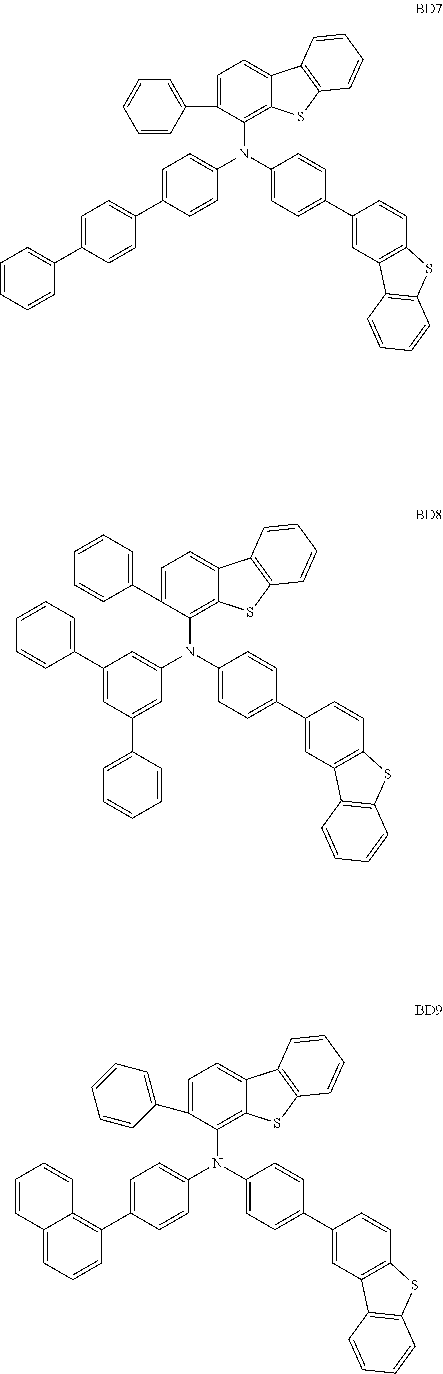 [SMILES: c1ccc(-c2ccc(-c3ccc(N(c4ccc(-c5ccc6sc7ccccc7c6c5)cc4)c4c(-c5ccccc5)ccc5c4sc4ccccc45)cc3)cc2)cc1 ; c1ccc(-c2cc(-c3ccccc3)cc(N(c3ccc(-c4ccc5sc6ccccc6c5c4)cc3)c3c(-c4ccccc4)ccc4c3sc3ccccc34)c2)cc1 ; c1ccc(-c2ccc3c(sc4ccccc43)c2N(c2ccc(-c3ccc4sc5ccccc5c4c3)cc2)c2ccc(-c3cccc4ccccc34)cc2)cc1]

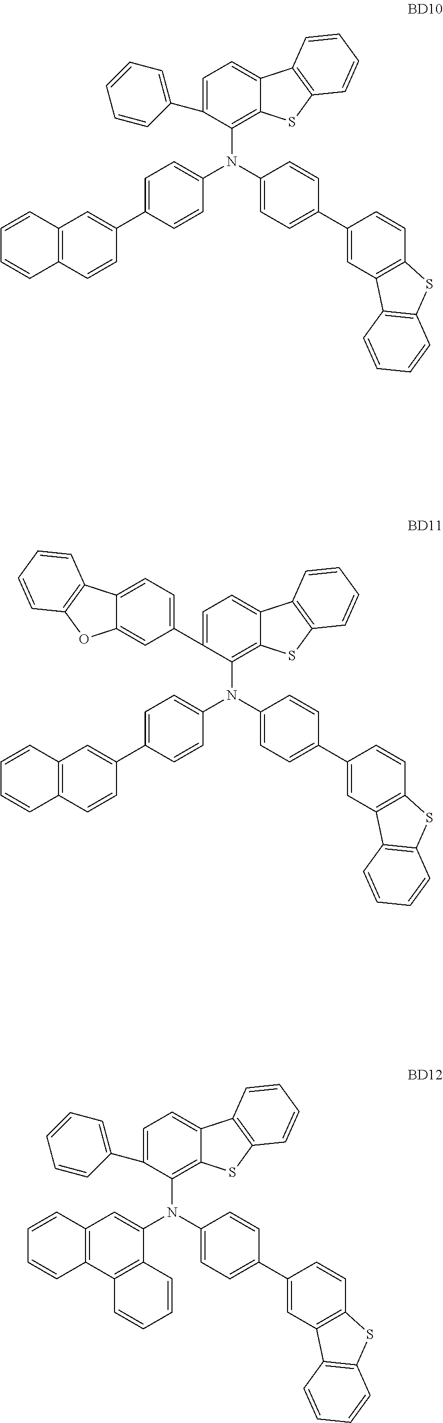 [SMILES: c1ccc(-c2ccc3c(sc4ccccc43)c2N(c2ccc(-c3ccc4ccccc4c3)cc2)c2ccc(-c3ccc4sc5ccccc5c4c3)cc2)cc1 ; c1ccc2cc(-c3ccc(N(c4ccc(-c5ccc6sc7ccccc7c6c5)cc4)c4c(-c5ccc6c(c5)oc5ccccc56)ccc5c4sc4ccccc45)cc3)ccc2c1 ; c1ccc(-c2ccc3c(sc4ccccc43)c2N(c2ccc(-c3ccc4sc5ccccc5c4c3)cc2)c2cc3ccccc3c3ccccc23)cc1]

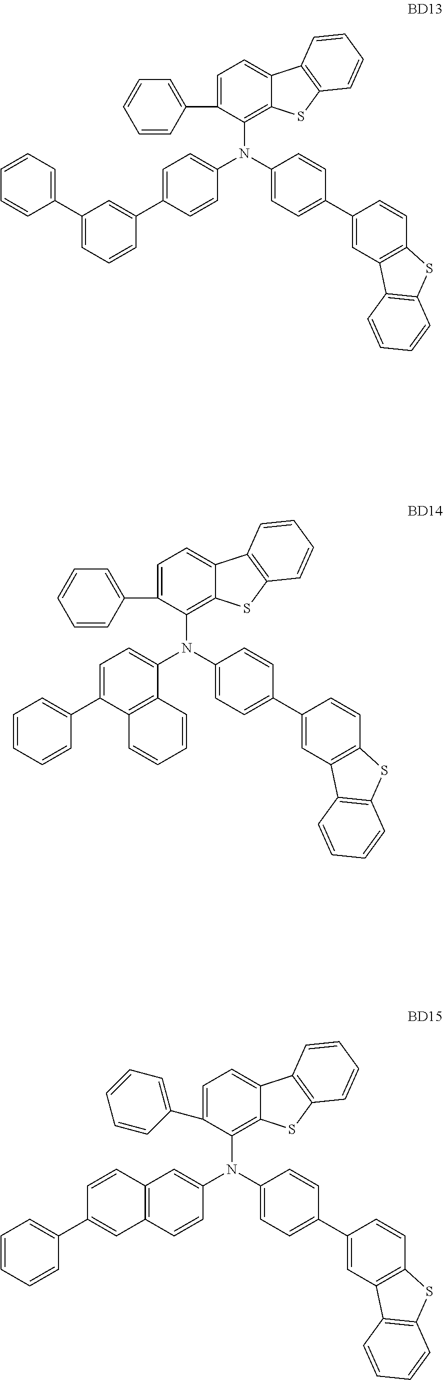 [SMILES: c1ccc(-c2cccc(-c3ccc(N(c4ccc(-c5ccc6sc7ccccc7c6c5)cc4)c4c(-c5ccccc5)ccc5c4sc4ccccc45)cc3)c2)cc1 ; c1ccc(-c2ccc3c(sc4ccccc43)c2N(c2ccc(-c3ccc4sc5ccccc5c4c3)cc2)c2ccc(-c3ccccc3)c3ccccc23)cc1 ; c1ccc(-c2ccc3cc(N(c4ccc(-c5ccc6sc7ccccc7c6c5)cc4)c4c(-c5ccccc5)ccc5c4sc4ccccc45)ccc3c2)cc1]

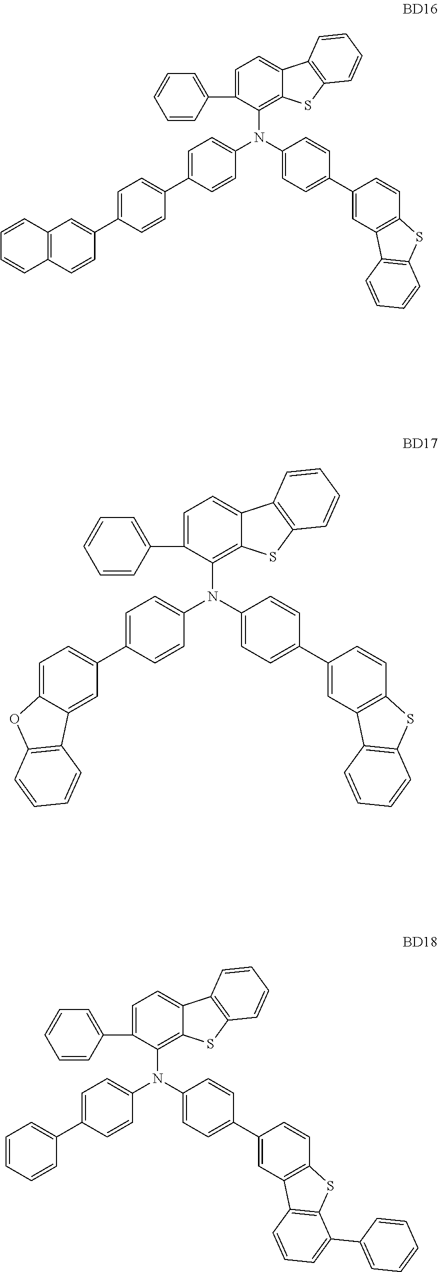 [SMILES: c1ccc(-c2ccc3c(sc4ccccc43)c2N(c2ccc(-c3ccc(-c4ccc5ccccc5c4)cc3)cc2)c2ccc(-c3ccc4sc5ccccc5c4c3)cc2)cc1 ; c1ccc(-c2ccc3c(sc4ccccc43)c2N(c2ccc(-c3ccc4oc5ccccc5c4c3)cc2)c2ccc(-c3ccc4sc5ccccc5c4c3)cc2)cc1 ; c1ccc(-c2ccc(N(c3ccc(-c4ccc5sc6c(-c7ccccc7)cccc6c5c4)cc3)c3c(-c4ccccc4)ccc4c3sc3ccccc34)cc2)cc1]

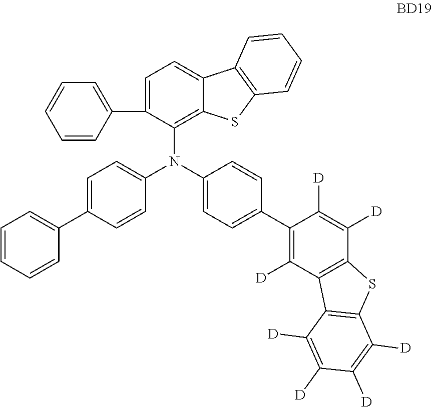 [SMILES: [2H]c1c([2H])c([2H])c2c(sc3c([2H])c([2H])c(-c4ccc(N(c5ccc(-c6ccccc6)cc5)c5c(-c6ccccc6)ccc6c5sc5ccccc56)cc4)c([2H])c32)c1[2H]]

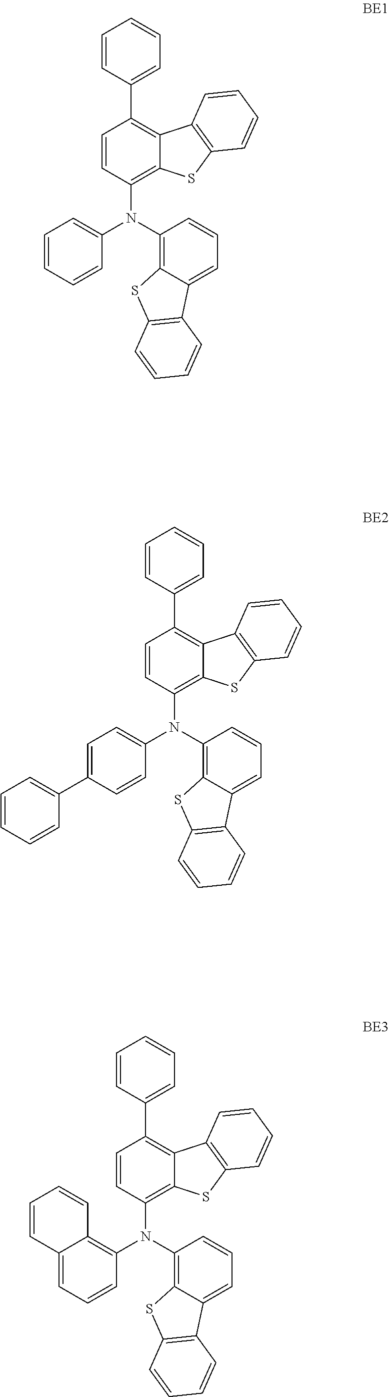 [SMILES: c1ccc(-c2ccc(N(c3ccccc3)c3cccc4c3sc3ccccc34)c3sc4ccccc4c23)cc1 ; c1ccc(-c2ccc(N(c3cccc4c3sc3ccccc34)c3ccc(-c4ccccc4)c4c3sc3ccccc34)cc2)cc1 ; c1ccc(-c2ccc(N(c3cccc4ccccc34)c3cccc4c3sc3ccccc34)c3sc4ccccc4c23)cc1]

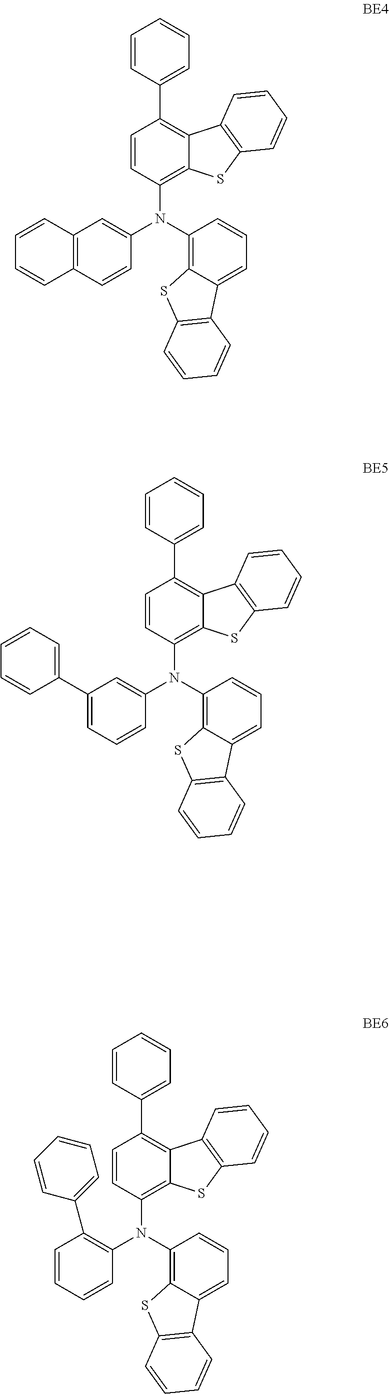 [SMILES: c1ccc(-c2ccc(N(c3ccc4ccccc4c3)c3cccc4c3sc3ccccc34)c3sc4ccccc4c23)cc1 ; c1ccc(-c2cccc(N(c3cccc4c3sc3ccccc34)c3ccc(-c4ccccc4)c4c3sc3ccccc34)c2)cc1 ; c1ccc(-c2ccccc2N(c2cccc3c2sc2ccccc23)c2ccc(-c3ccccc3)c3c2sc2ccccc23)cc1]

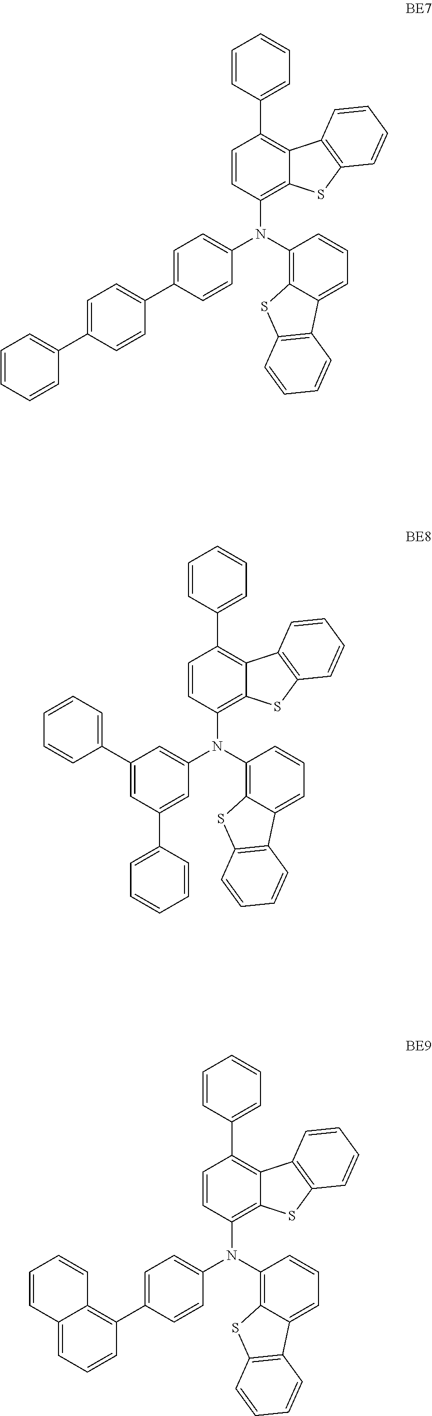 [SMILES: c1ccc(-c2ccc(-c3ccc(N(c4cccc5c4sc4ccccc45)c4ccc(-c5ccccc5)c5c4sc4ccccc45)cc3)cc2)cc1 ; c1ccc(-c2cc(-c3ccccc3)cc(N(c3cccc4c3sc3ccccc34)c3ccc(-c4ccccc4)c4c3sc3ccccc34)c2)cc1 ; c1ccc(-c2ccc(N(c3ccc(-c4cccc5ccccc45)cc3)c3cccc4c3sc3ccccc34)c3sc4ccccc4c23)cc1]

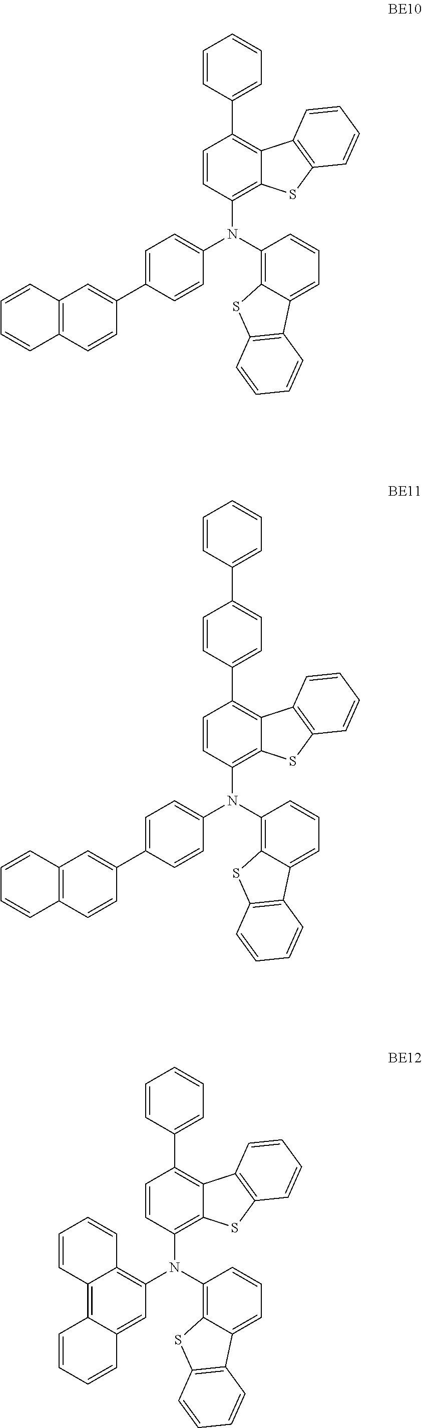 [SMILES: c1ccc(-c2ccc(N(c3ccc(-c4ccc5ccccc5c4)cc3)c3cccc4c3sc3ccccc34)c3sc4ccccc4c23)cc1 ; c1ccc(-c2ccc(-c3ccc(N(c4ccc(-c5ccc6ccccc6c5)cc4)c4cccc5c4sc4ccccc45)c4sc5ccccc5c34)cc2)cc1 ; c1ccc(-c2ccc(N(c3cc4ccccc4c4ccccc34)c3cccc4c3sc3ccccc34)c3sc4ccccc4c23)cc1]

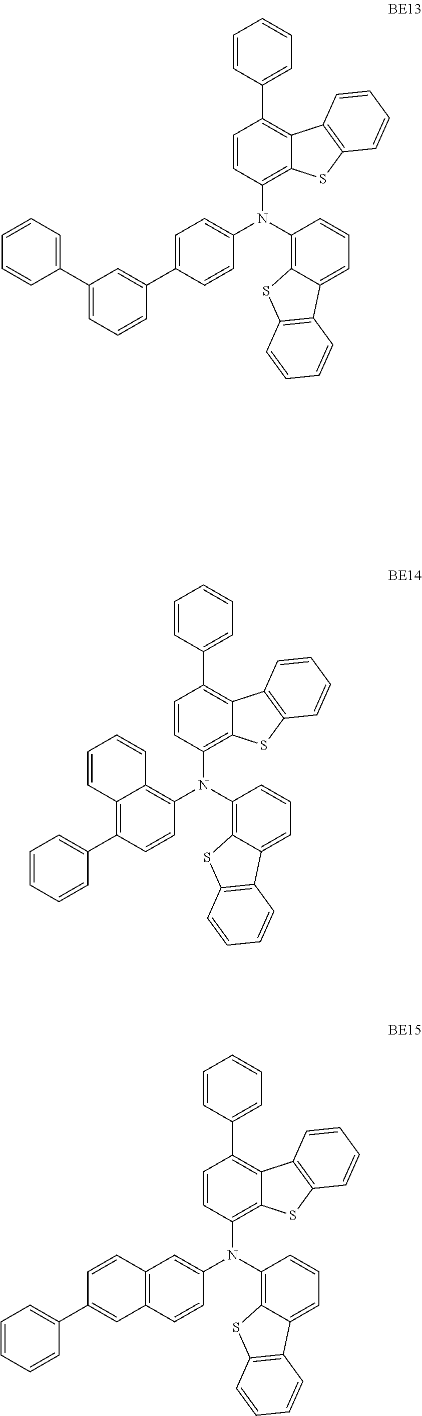 [SMILES: c1ccc(-c2cccc(-c3ccc(N(c4cccc5c4sc4ccccc45)c4ccc(-c5ccccc5)c5c4sc4ccccc45)cc3)c2)cc1 ; c1ccc(-c2ccc(N(c3cccc4c3sc3ccccc34)c3ccc(-c4ccccc4)c4c3sc3ccccc34)c3ccccc23)cc1 ; c1ccc(-c2ccc3cc(N(c4cccc5c4sc4ccccc45)c4ccc(-c5ccccc5)c5c4sc4ccccc45)ccc3c2)cc1]

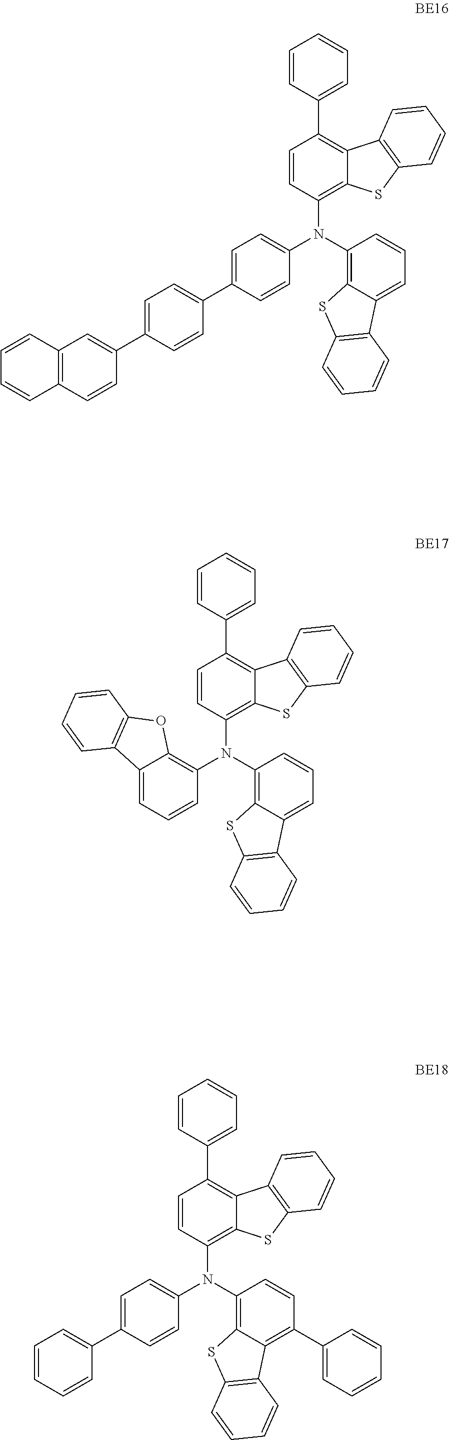 [SMILES: c1ccc(-c2ccc(N(c3ccc(-c4ccc(-c5ccc6ccccc6c5)cc4)cc3)c3cccc4c3sc3ccccc34)c3sc4ccccc4c23)cc1 ; c1ccc(-c2ccc(N(c3cccc4c3oc3ccccc34)c3cccc4c3sc3ccccc34)c3sc4ccccc4c23)cc1 ; c1ccc(-c2ccc(N(c3ccc(-c4ccccc4)c4c3sc3ccccc34)c3ccc(-c4ccccc4)c4c3sc3ccccc34)cc2)cc1]

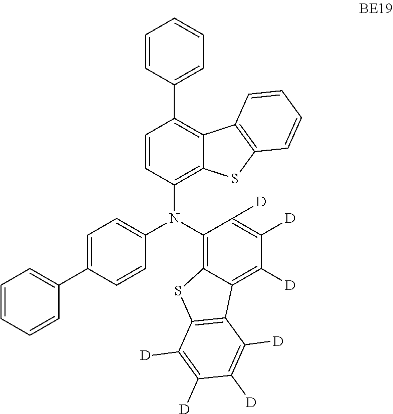 [SMILES: [2H]c1c([2H])c([2H])c2c(sc3c(N(c4ccc(-c5ccccc5)cc4)c4ccc(-c5ccccc5)c5c4sc4ccccc45)c([2H])c([2H])c([2H])c32)c1[2H]]

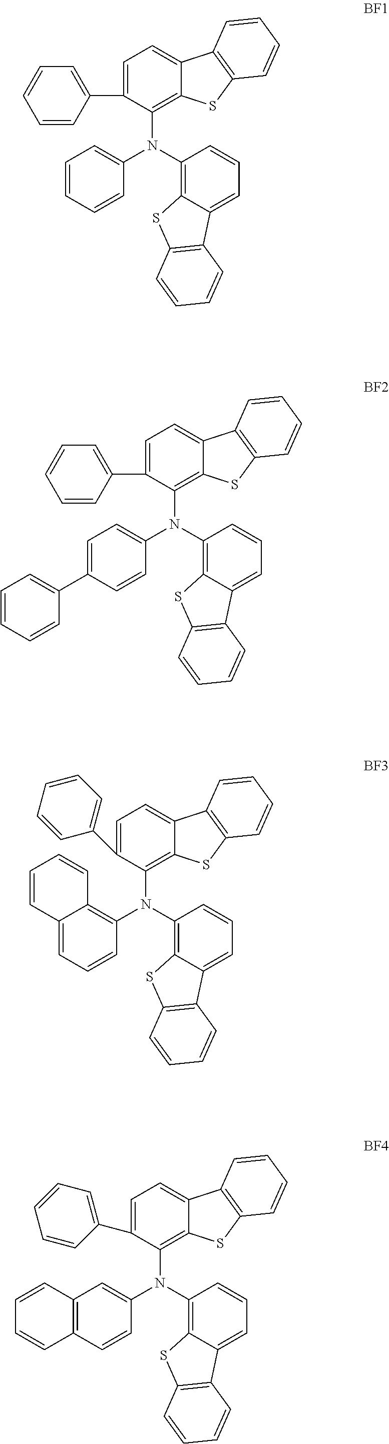 [SMILES: c1ccc(-c2ccc3c(sc4ccccc43)c2N(c2ccccc2)c2cccc3c2sc2ccccc23)cc1 ; c1ccc(-c2ccc(N(c3cccc4c3sc3ccccc34)c3c(-c4ccccc4)ccc4c3sc3ccccc34)cc2)cc1 ; c1ccc(-c2ccc3c(sc4ccccc43)c2N(c2cccc3ccccc23)c2cccc3c2sc2ccccc23)cc1 ; c1ccc(-c2ccc3c(sc4ccccc43)c2N(c2ccc3ccccc3c2)c2cccc3c2sc2ccccc23)cc1]

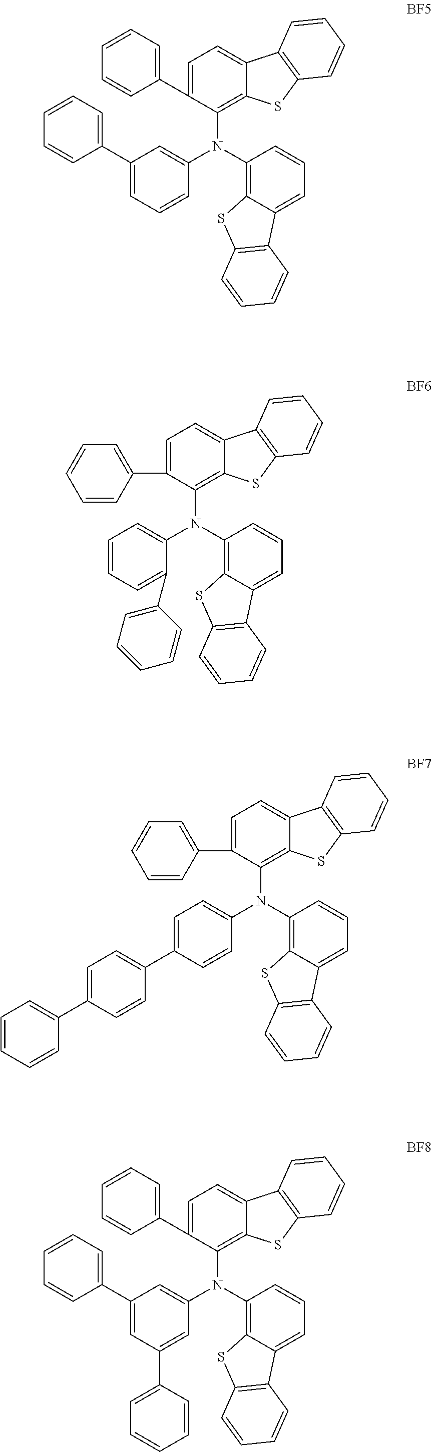 [SMILES: c1ccc(-c2cccc(N(c3cccc4c3sc3ccccc34)c3c(-c4ccccc4)ccc4c3sc3ccccc34)c2)cc1 ; c1ccc(-c2ccccc2N(c2cccc3c2sc2ccccc23)c2c(-c3ccccc3)ccc3c2sc2ccccc23)cc1 ; c1ccc(-c2ccc(-c3ccc(N(c4cccc5c4sc4ccccc45)c4c(-c5ccccc5)ccc5c4sc4ccccc45)cc3)cc2)cc1 ; c1ccc(-c2cc(-c3ccccc3)cc(N(c3cccc4c3sc3ccccc34)c3c(-c4ccccc4)ccc4c3sc3ccccc34)c2)cc1]

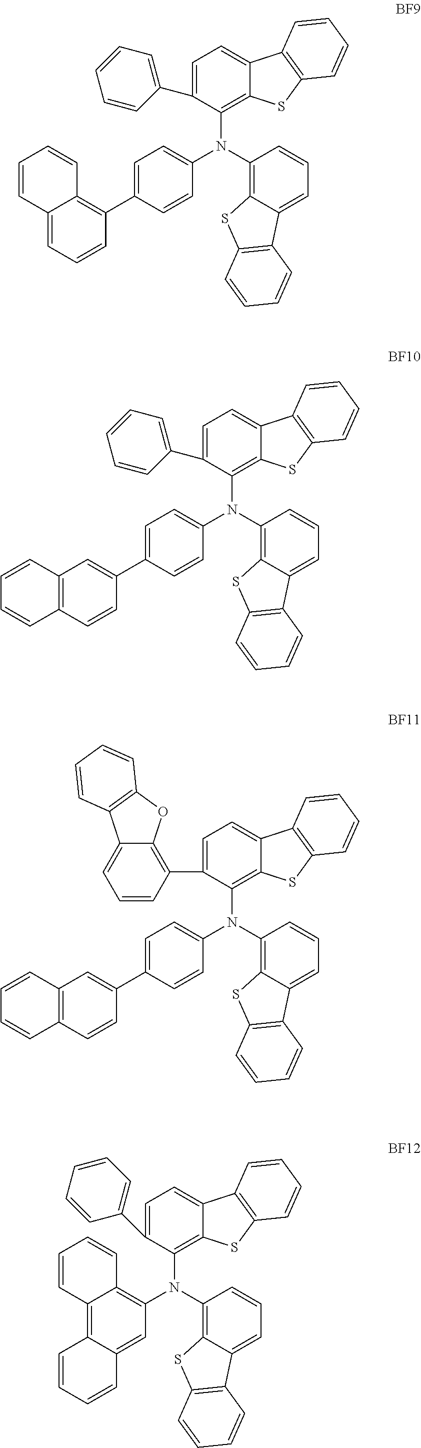 [SMILES: c1ccc(-c2ccc3c(sc4ccccc43)c2N(c2ccc(-c3cccc4ccccc34)cc2)c2cccc3c2sc2ccccc23)cc1 ; c1ccc(-c2ccc3c(sc4ccccc43)c2N(c2ccc(-c3ccc4ccccc4c3)cc2)c2cccc3c2sc2ccccc23)cc1 ; c1ccc2cc(-c3ccc(N(c4cccc5c4sc4ccccc45)c4c(-c5cccc6c5oc5ccccc56)ccc5c4sc4ccccc45)cc3)ccc2c1 ; c1ccc(-c2ccc3c(sc4ccccc43)c2N(c2cc3ccccc3c3ccccc23)c2cccc3c2sc2ccccc23)cc1]

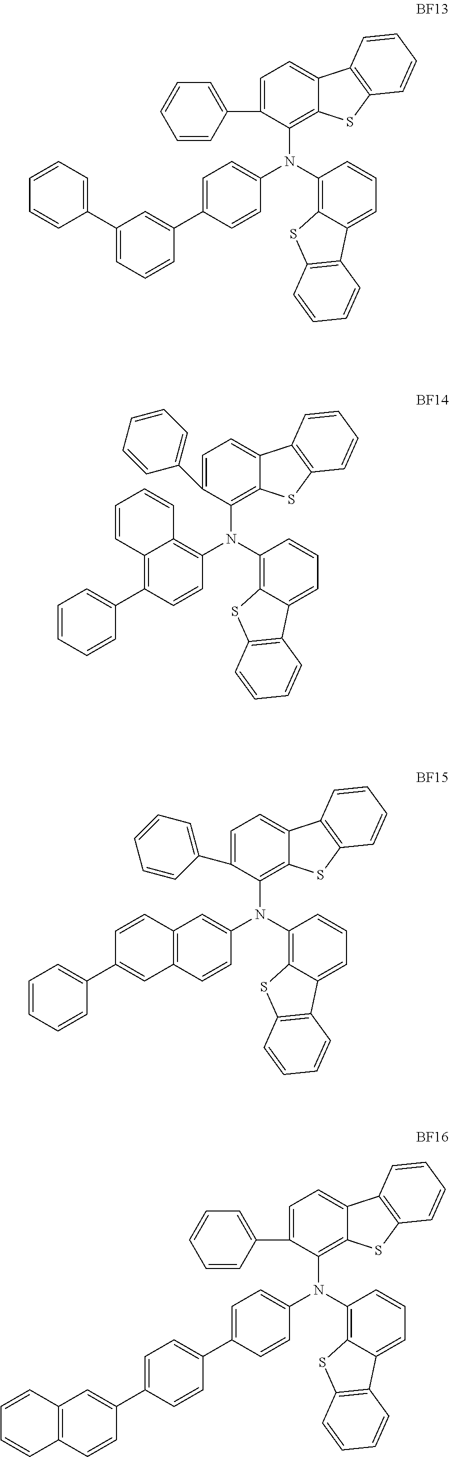 [SMILES: c1ccc(-c2cccc(-c3ccc(N(c4cccc5c4sc4ccccc45)c4c(-c5ccccc5)ccc5c4sc4ccccc45)cc3)c2)cc1 ; c1ccc(-c2ccc3c(sc4ccccc43)c2N(c2ccc(-c3ccccc3)c3ccccc23)c2cccc3c2sc2ccccc23)cc1 ; c1ccc(-c2ccc3cc(N(c4cccc5c4sc4ccccc45)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2cccc3c2sc2ccccc23)cc1]

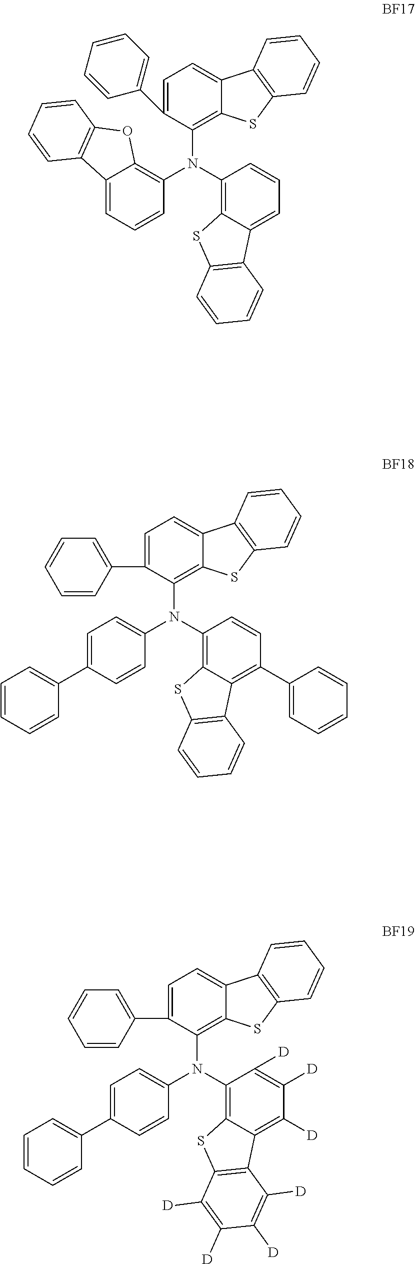 [SMILES: c1ccc(-c2ccc3c(sc4ccccc43)c2N(c2cccc3c2oc2ccccc23)c2cccc3c2sc2ccccc23)cc1 ; c1ccc(-c2ccc(N(c3c(-c4ccccc4)ccc4c3sc3ccccc34)c3ccc(-c4ccccc4)c4c3sc3ccccc34)cc2)cc1 ; [2H]c1c([2H])c([2H])c2c(sc3c(N(c4ccc(-c5ccccc5)cc4)c4c(-c5ccccc5)ccc5c4sc4ccccc45)c([2H])c([2H])c([2H])c32)c1[2H]]

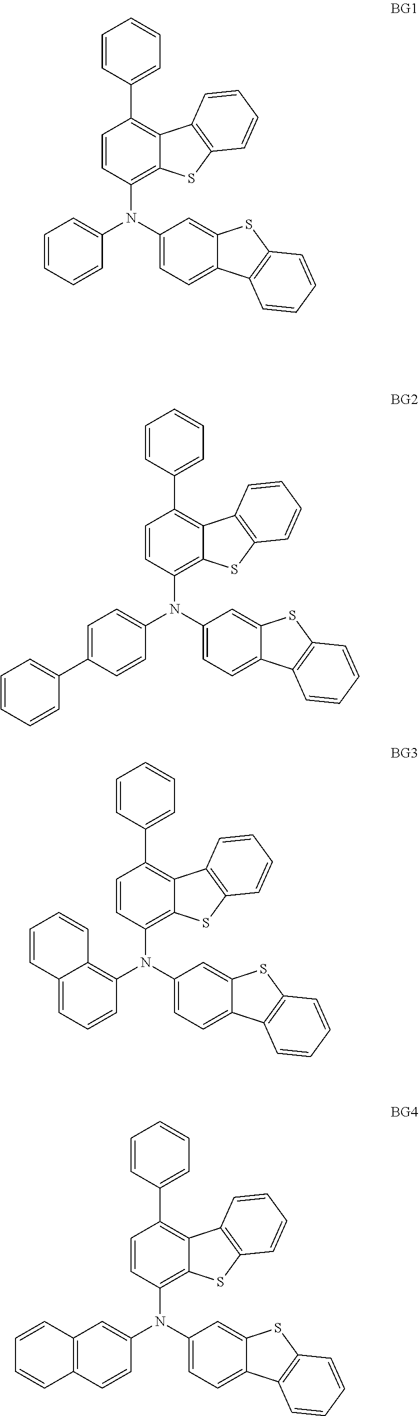 [SMILES: c1ccc(-c2ccc(N(c3ccccc3)c3ccc4c(c3)sc3ccccc34)c3sc4ccccc4c23)cc1 ; c1ccc(-c2ccc(N(c3ccc4c(c3)sc3ccccc34)c3ccc(-c4ccccc4)c4c3sc3ccccc34)cc2)cc1 ; c1ccc(-c2ccc(N(c3ccc4c(c3)sc3ccccc34)c3cccc4ccccc34)c3sc4ccccc4c23)cc1 ; c1ccc(-c2ccc(N(c3ccc4ccccc4c3)c3ccc4c(c3)sc3ccccc34)c3sc4ccccc4c23)cc1]

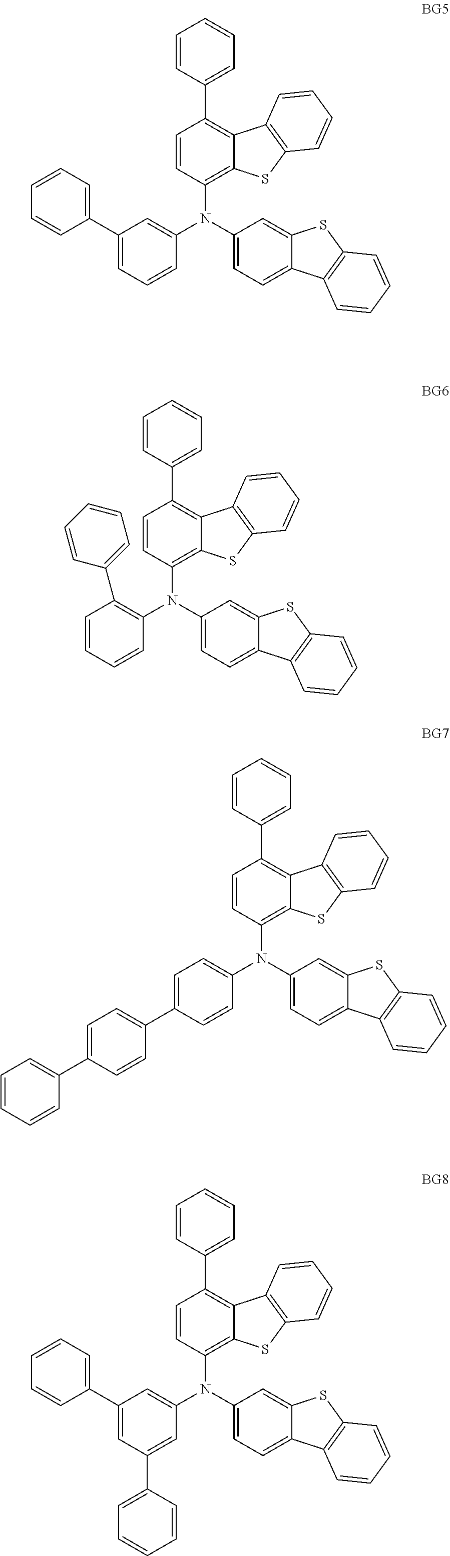 [SMILES: c1ccc(-c2cccc(N(c3ccc4c(c3)sc3ccccc34)c3ccc(-c4ccccc4)c4c3sc3ccccc34)c2)cc1 ; c1ccc(-c2ccccc2N(c2ccc3c(c2)sc2ccccc23)c2ccc(-c3ccccc3)c3c2sc2ccccc23)cc1 ; c1ccc(-c2ccc(-c3ccc(N(c4ccc5c(c4)sc4ccccc45)c4ccc(-c5ccccc5)c5c4sc4ccccc45)cc3)cc2)cc1 ; c1ccc(-c2cc(-c3ccccc3)cc(N(c3ccc4c(c3)sc3ccccc34)c3ccc(-c4ccccc4)c4c3sc3ccccc34)c2)cc1]

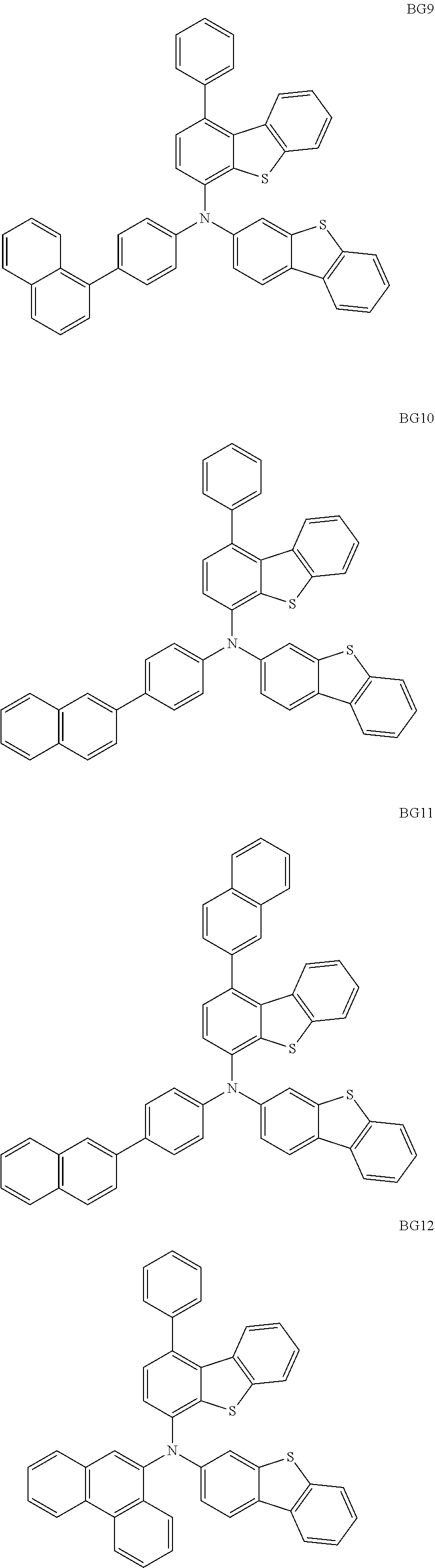 [SMILES: c1ccc(-c2ccc(N(c3ccc(-c4cccc5ccccc45)cc3)c3ccc4c(c3)sc3ccccc34)c3sc4ccccc4c23)cc1 ; c1ccc(-c2ccc(N(c3ccc(-c4ccc5ccccc5c4)cc3)c3ccc4c(c3)sc3ccccc34)c3sc4ccccc4c23)cc1 ; c1ccc2cc(-c3ccc(N(c4ccc5c(c4)sc4ccccc45)c4ccc(-c5ccc6ccccc6c5)c5c4sc4ccccc45)cc3)ccc2c1 ; c1ccc(-c2ccc(N(c3ccc4c(c3)sc3ccccc34)c3cc4ccccc4c4ccccc34)c3sc4ccccc4c23)cc1]

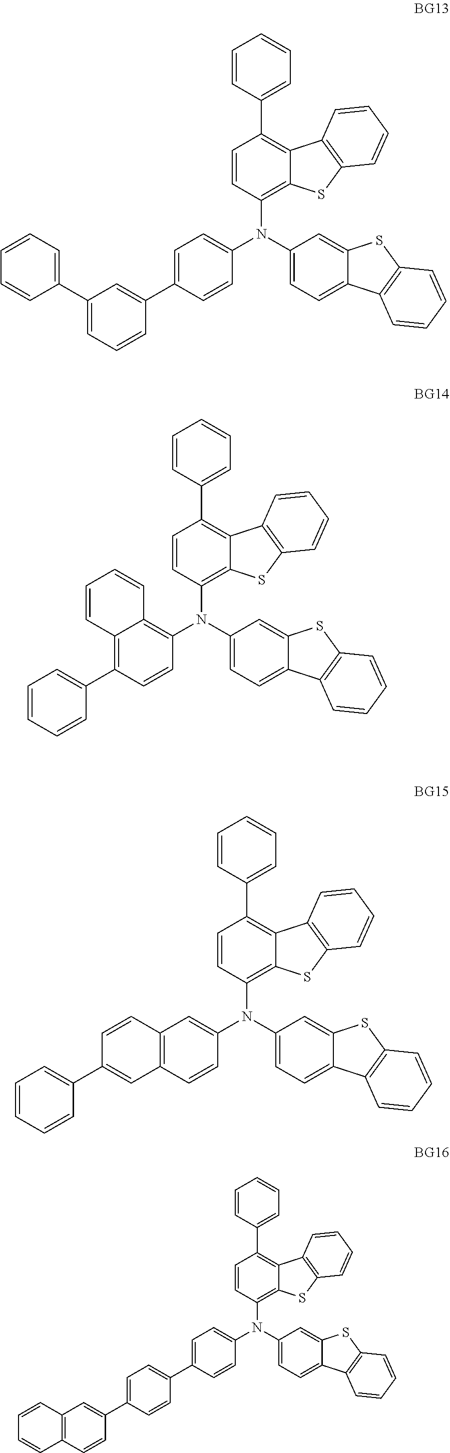 [SMILES: c1ccc(-c2cccc(-c3ccc(N(c4ccc5c(c4)sc4ccccc45)c4ccc(-c5ccccc5)c5c4sc4ccccc45)cc3)c2)cc1 ; c1ccc(-c2ccc(N(c3ccc4c(c3)sc3ccccc34)c3ccc(-c4ccccc4)c4c3sc3ccccc34)c3ccccc23)cc1 ; c1ccc(-c2ccc3cc(N(c4ccc5c(c4)sc4ccccc45)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc4c(c3)sc3ccccc34)c3sc4ccccc4c23)cc1]

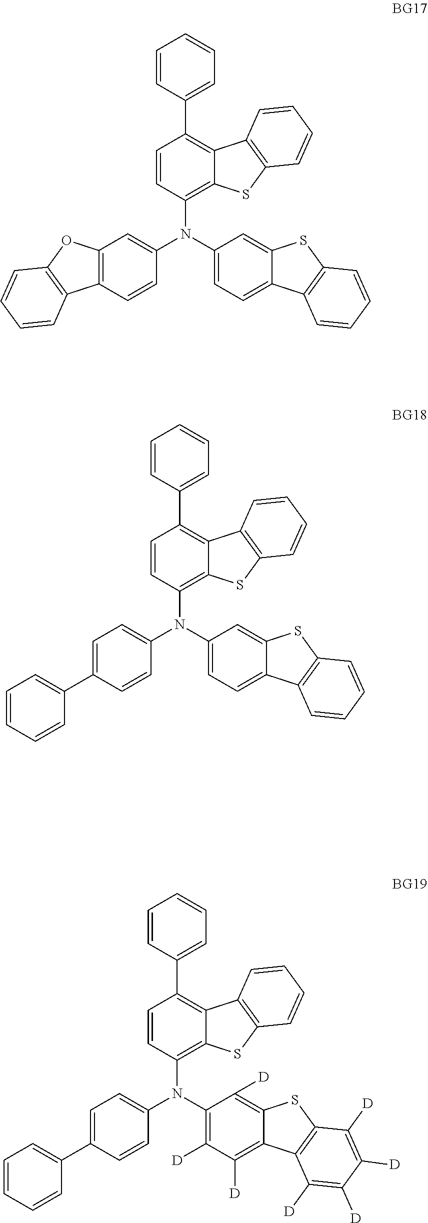 [SMILES: c1ccc(-c2ccc(N(c3ccc4c(c3)oc3ccccc34)c3ccc4c(c3)sc3ccccc34)c3sc4ccccc4c23)cc1 ; c1ccc(-c2ccc(N(c3ccc4c(c3)sc3ccccc34)c3ccc(-c4ccccc4)c4c3sc3ccccc34)cc2)cc1 ; [2H]c1c([2H])c([2H])c2c(sc3c([2H])c(N(c4ccc(-c5ccccc5)cc4)c4ccc(-c5ccccc5)c5c4sc4ccccc45)c([2H])c([2H])c32)c1[2H]]

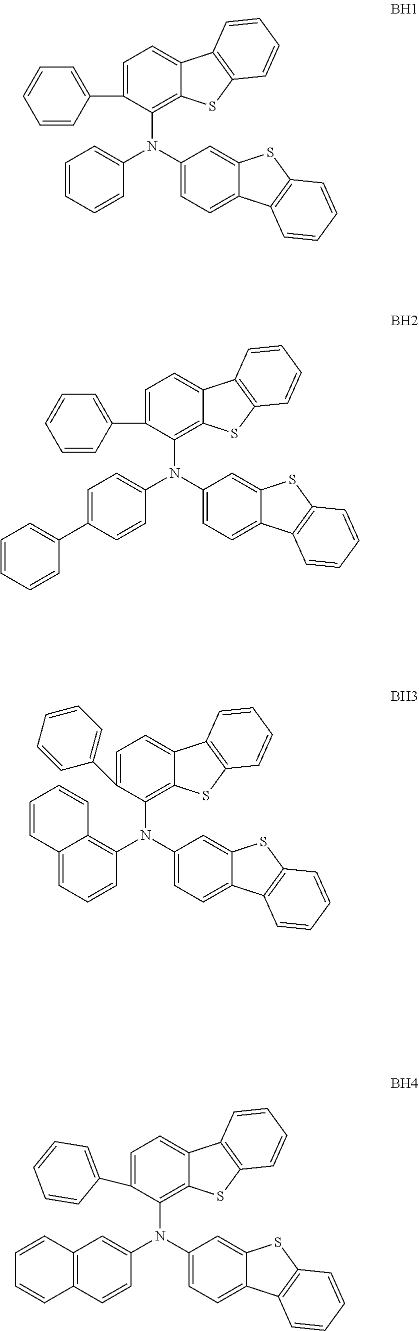 [SMILES: c1ccc(-c2ccc3c(sc4ccccc43)c2N(c2ccccc2)c2ccc3c(c2)sc2ccccc23)cc1 ; c1ccc(-c2ccc(N(c3ccc4c(c3)sc3ccccc34)c3c(-c4ccccc4)ccc4c3sc3ccccc34)cc2)cc1 ; c1ccc(-c2ccc3c(sc4ccccc43)c2N(c2ccc3c(c2)sc2ccccc23)c2cccc3ccccc23)cc1 ; c1ccc(-c2ccc3c(sc4ccccc43)c2N(c2ccc3ccccc3c2)c2ccc3c(c2)sc2ccccc23)cc1]

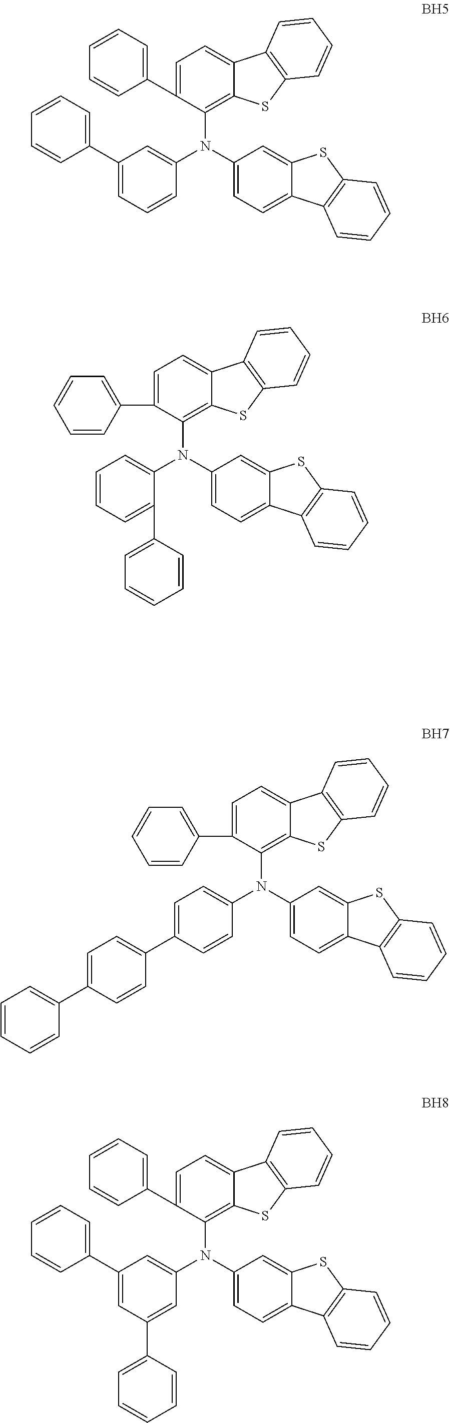 [SMILES: c1ccc(-c2cccc(N(c3ccc4c(c3)sc3ccccc34)c3c(-c4ccccc4)ccc4c3sc3ccccc34)c2)cc1 ; c1ccc(-c2ccccc2N(c2ccc3c(c2)sc2ccccc23)c2c(-c3ccccc3)ccc3c2sc2ccccc23)cc1 ; c1ccc(-c2ccc(-c3ccc(N(c4ccc5c(c4)sc4ccccc45)c4c(-c5ccccc5)ccc5c4sc4ccccc45)cc3)cc2)cc1 ; c1ccc(-c2cc(-c3ccccc3)cc(N(c3ccc4c(c3)sc3ccccc34)c3c(-c4ccccc4)ccc4c3sc3ccccc34)c2)cc1]

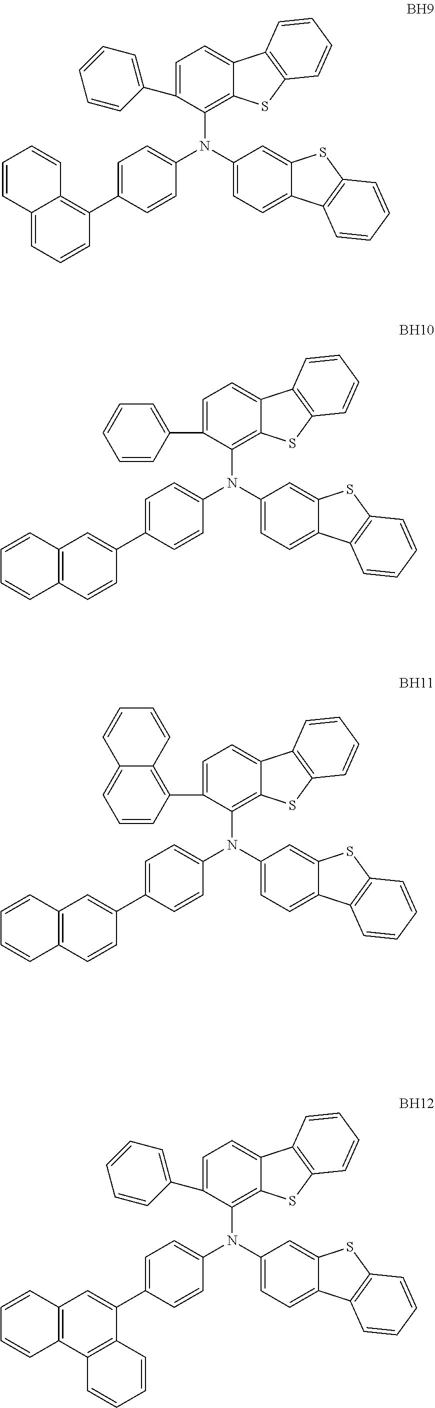 [SMILES: c1ccc(-c2ccc3c(sc4ccccc43)c2N(c2ccc(-c3cccc4ccccc34)cc2)c2ccc3c(c2)sc2ccccc23)cc1 ; c1ccc(-c2ccc3c(sc4ccccc43)c2N(c2ccc(-c3ccc4ccccc4c3)cc2)c2ccc3c(c2)sc2ccccc23)cc1 ; c1ccc2cc(-c3ccc(N(c4ccc5c(c4)sc4ccccc45)c4c(-c5cccc6ccccc56)ccc5c4sc4ccccc45)cc3)ccc2c1 ; c1ccc(-c2ccc3c(sc4ccccc43)c2N(c2ccc(-c3cc4ccccc4c4ccccc34)cc2)c2ccc3c(c2)sc2ccccc23)cc1]

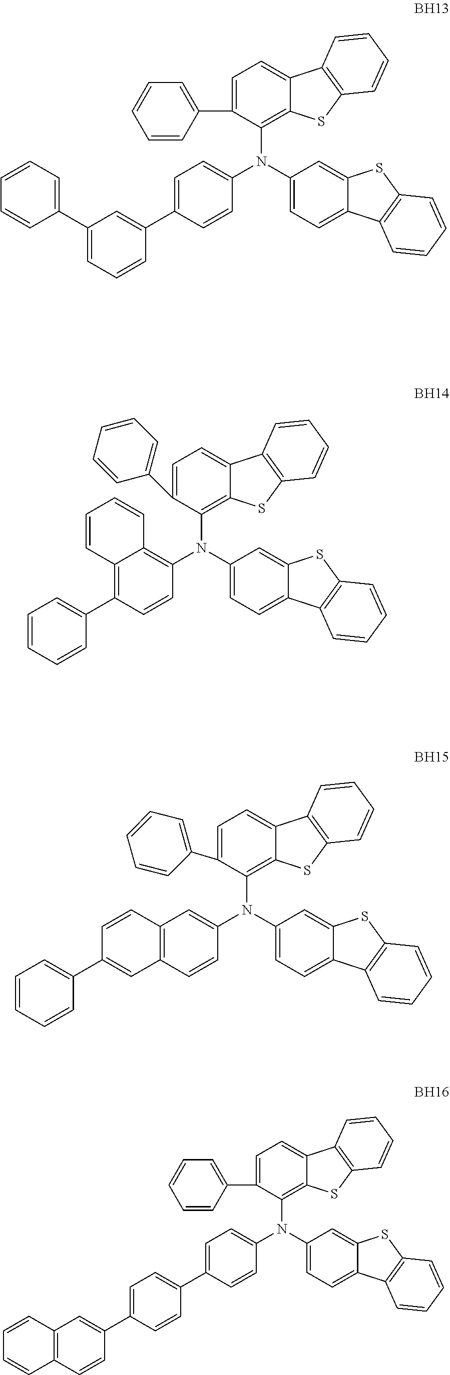 [SMILES: c1ccc(-c2cccc(-c3ccc(N(c4ccc5c(c4)sc4ccccc45)c4c(-c5ccccc5)ccc5c4sc4ccccc45)cc3)c2)cc1 ; c1ccc(-c2ccc3c(sc4ccccc43)c2N(c2ccc3c(c2)sc2ccccc23)c2ccc(-c3ccccc3)c3ccccc23)cc1 ; c1ccc(-c2ccc3cc(N(c4ccc5c(c4)sc4ccccc45)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc3c(c2)sc2ccccc23)cc1]

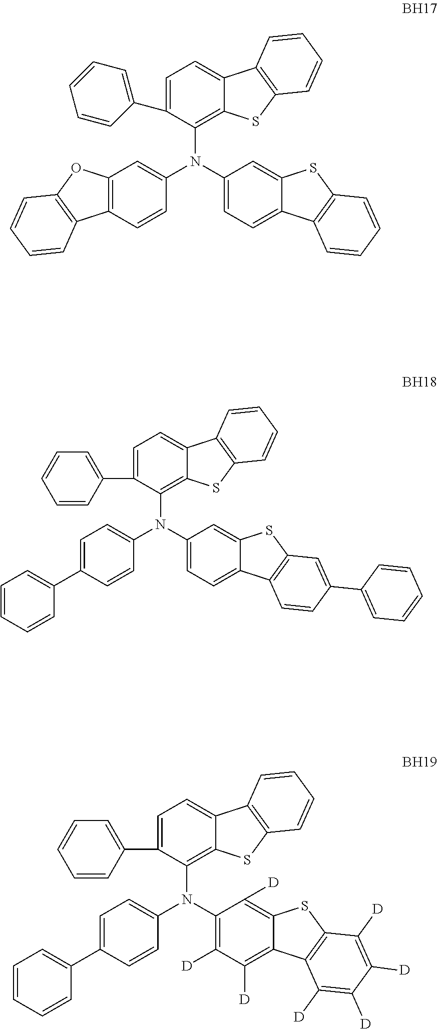 [SMILES: c1ccc(-c2ccc3c(sc4ccccc43)c2N(c2ccc3c(c2)oc2ccccc23)c2ccc3c(c2)sc2ccccc23)cc1 ; c1ccc(-c2ccc(N(c3ccc4c(c3)sc3cc(-c5ccccc5)ccc34)c3c(-c4ccccc4)ccc4c3sc3ccccc34)cc2)cc1 ; [2H]c1c([2H])c([2H])c2c(sc3c([2H])c(N(c4ccc(-c5ccccc5)cc4)c4c(-c5ccccc5)ccc5c4sc4ccccc45)c([2H])c([2H])c32)c1[2H]]

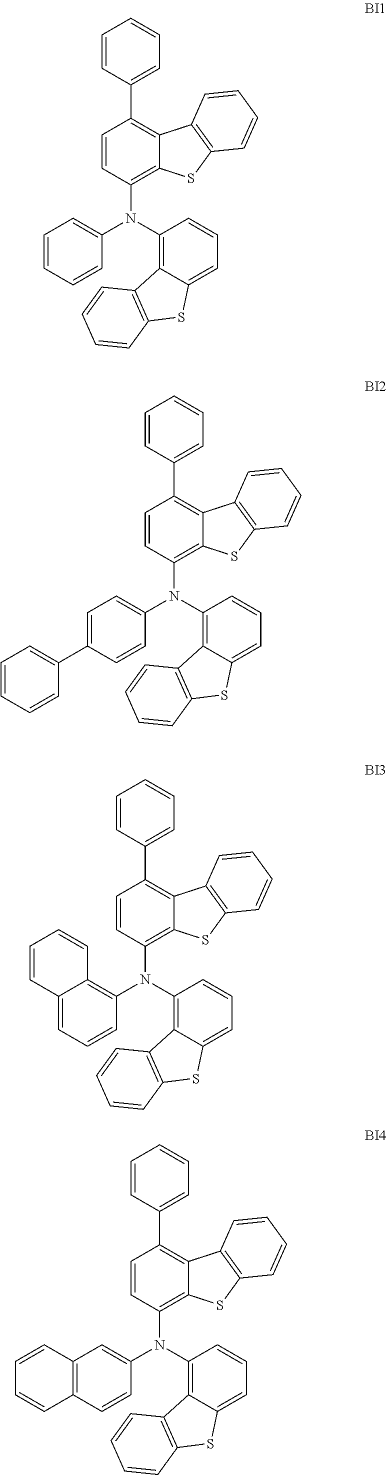

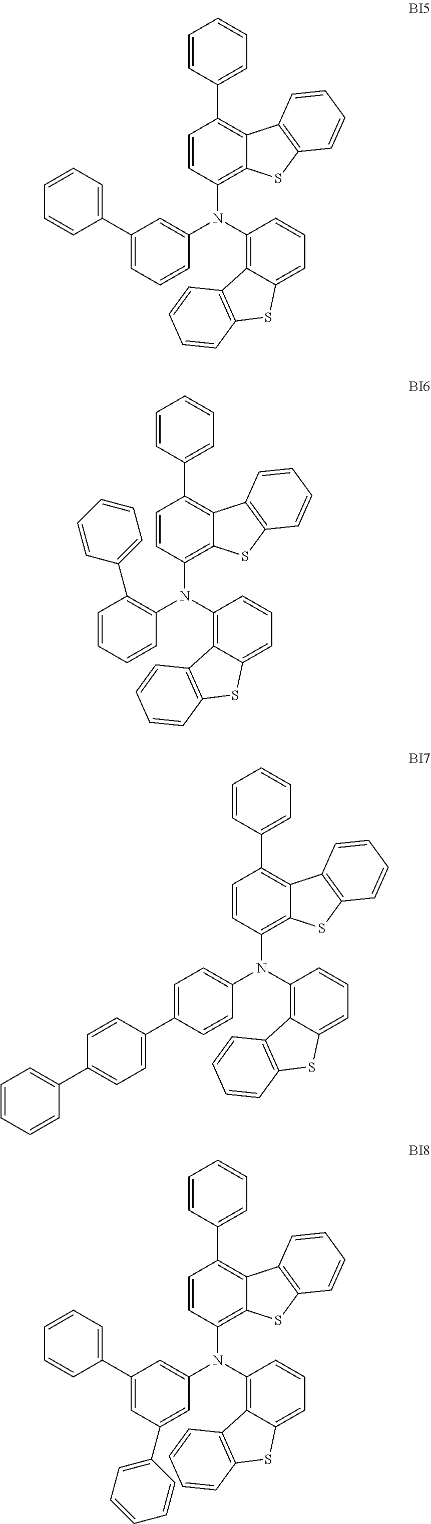 [SMILES: c1ccc(-c2cccc(N(c3ccc(-c4ccccc4)c4c3sc3ccccc34)c3cccc4sc5ccccc5c34)c2)cc1 ; c1ccc(-c2ccccc2N(c2ccc(-c3ccccc3)c3c2sc2ccccc23)c2cccc3sc4ccccc4c23)cc1 ; c1ccc(-c2ccc(-c3ccc(N(c4ccc(-c5ccccc5)c5c4sc4ccccc45)c4cccc5sc6ccccc6c45)cc3)cc2)cc1 ; c1ccc(-c2cc(-c3ccccc3)cc(N(c3ccc(-c4ccccc4)c4c3sc3ccccc34)c3cccc4sc5ccccc5c34)c2)cc1]

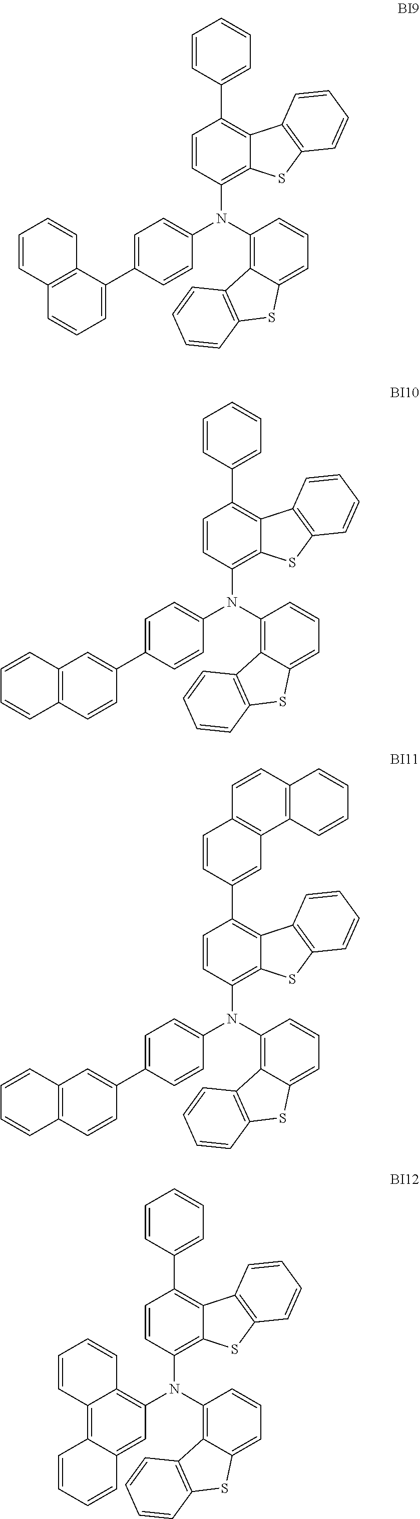 [SMILES: c1ccc(-c2ccc(N(c3ccc(-c4cccc5ccccc45)cc3)c3cccc4sc5ccccc5c34)c3sc4ccccc4c23)cc1 ; c1ccc(-c2ccc(N(c3ccc(-c4ccc5ccccc5c4)cc3)c3cccc4sc5ccccc5c34)c3sc4ccccc4c23)cc1 ; c1ccc2cc(-c3ccc(N(c4ccc(-c5ccc6ccc7ccccc7c6c5)c5c4sc4ccccc45)c4cccc5sc6ccccc6c45)cc3)ccc2c1 ; c1ccc(-c2ccc(N(c3cc4ccccc4c4ccccc34)c3cccc4sc5ccccc5c34)c3sc4ccccc4c23)cc1]

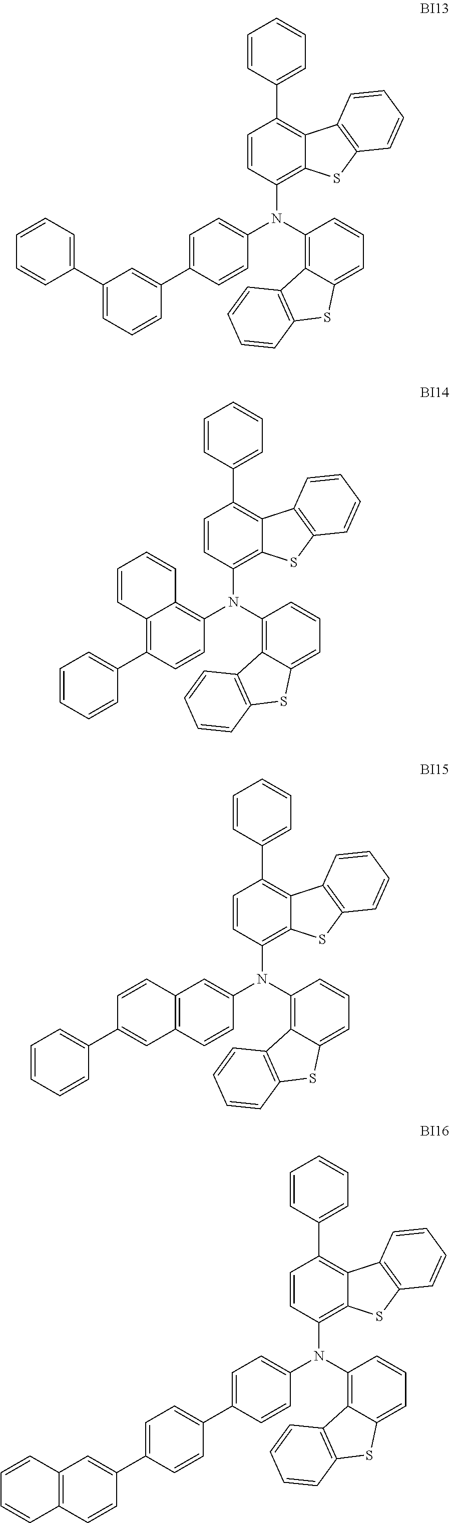 [SMILES: c1ccc(-c2cccc(-c3ccc(N(c4ccc(-c5ccccc5)c5c4sc4ccccc45)c4cccc5sc6ccccc6c45)cc3)c2)cc1 ; c1ccc(-c2ccc(N(c3ccc(-c4ccccc4)c4c3sc3ccccc34)c3cccc4sc5ccccc5c34)c3ccccc23)cc1 ; c1ccc(-c2ccc3cc(N(c4ccc(-c5ccccc5)c5c4sc4ccccc45)c4cccc5sc6ccccc6c45)ccc3c2)cc1 ; c1ccc(-c2ccc(N(c3ccc(-c4ccc(-c5ccc6ccccc6c5)cc4)cc3)c3cccc4sc5ccccc5c34)c3sc4ccccc4c23)cc1]

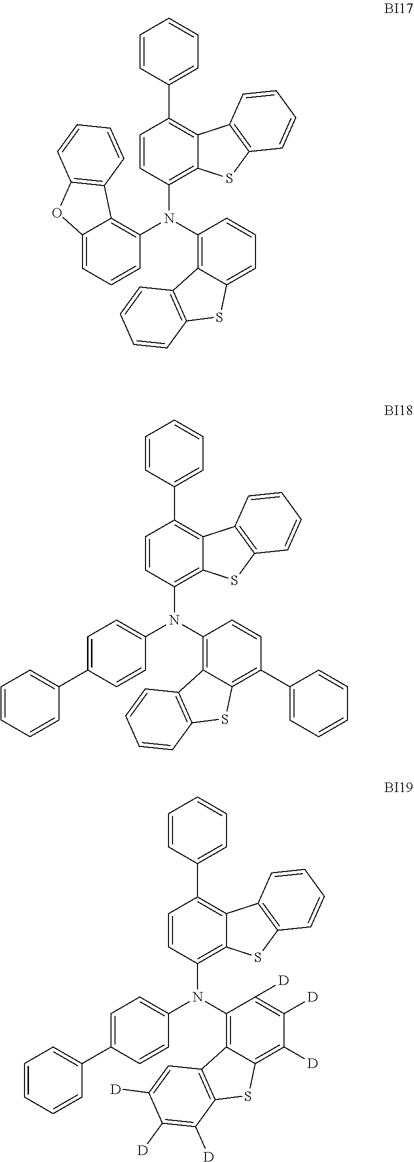 [SMILES: c1ccc(-c2ccc(N(c3cccc4oc5ccccc5c34)c3cccc4sc5ccccc5c34)c3sc4ccccc4c23)cc1 ; c1ccc(-c2ccc(N(c3ccc(-c4ccccc4)c4c3sc3ccccc34)c3ccc(-c4ccccc4)c4sc5ccccc5c34)cc2)cc1 ; [2H]c1cc2c(sc3c([2H])c([2H])c([2H])c(N(c4ccc(-c5ccccc5)cc4)c4ccc(-c5ccccc5)c5c4sc4ccccc45)c32)c([2H])c1[2H]]

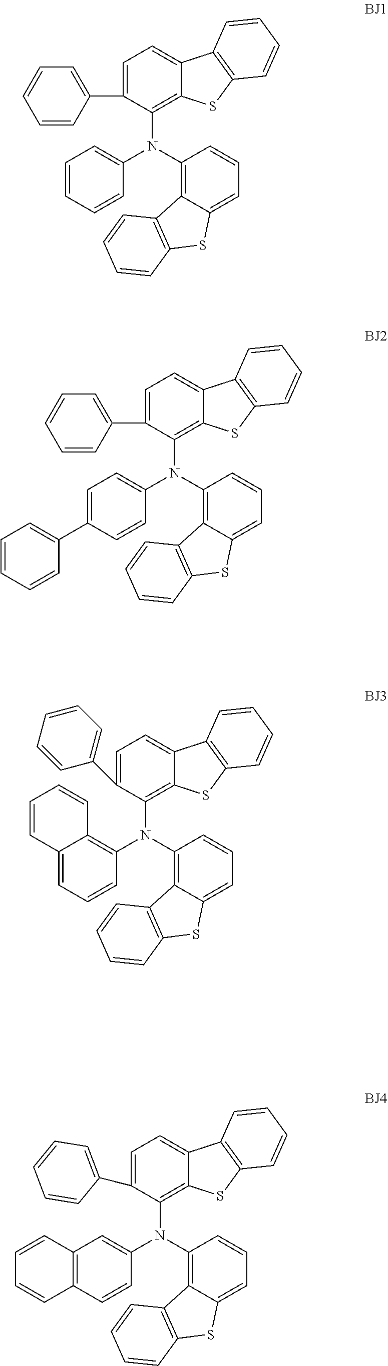 [SMILES: c1ccc(-c2ccc3c(sc4ccccc43)c2N(c2ccccc2)c2cccc3sc4ccccc4c23)cc1 ; c1ccc(-c2ccc(N(c3c(-c4ccccc4)ccc4c3sc3ccccc34)c3cccc4sc5ccccc5c34)cc2)cc1 ; c1ccc(-c2ccc3c(sc4ccccc43)c2N(c2cccc3ccccc23)c2cccc3sc4ccccc4c23)cc1 ; c1ccc(-c2ccc3c(sc4ccccc43)c2N(c2ccc3ccccc3c2)c2cccc3sc4ccccc4c23)cc1]

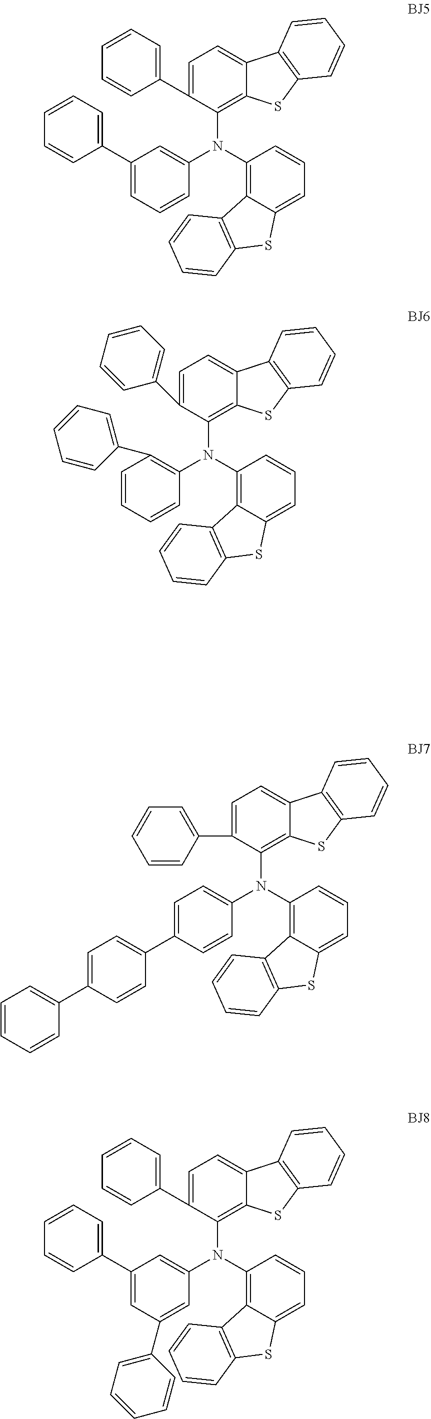 [SMILES: c1ccc(-c2cccc(N(c3c(-c4ccccc4)ccc4c3sc3ccccc34)c3cccc4sc5ccccc5c34)c2)cc1 ; c1ccc(-c2ccccc2N(c2c(-c3ccccc3)ccc3c2sc2ccccc23)c2cccc3sc4ccccc4c23)cc1 ; c1ccc(-c2ccc(-c3ccc(N(c4c(-c5ccccc5)ccc5c4sc4ccccc45)c4cccc5sc6ccccc6c45)cc3)cc2)cc1 ; c1ccc(-c2cc(-c3ccccc3)cc(N(c3c(-c4ccccc4)ccc4c3sc3ccccc34)c3cccc4sc5ccccc5c34)c2)cc1]

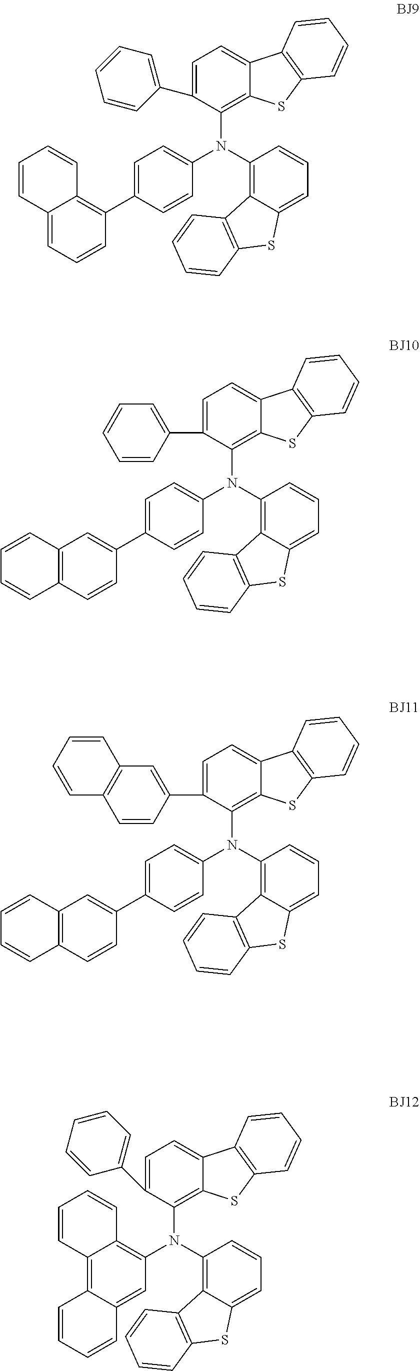 [SMILES: c1ccc(-c2ccc3c(sc4ccccc43)c2N(c2ccc(-c3cccc4ccccc34)cc2)c2cccc3sc4ccccc4c23)cc1 ; c1ccc(-c2ccc3c(sc4ccccc43)c2N(c2ccc(-c3ccc4ccccc4c3)cc2)c2cccc3sc4ccccc4c23)cc1 ; c1ccc2cc(-c3ccc(N(c4c(-c5ccc6ccccc6c5)ccc5c4sc4ccccc45)c4cccc5sc6ccccc6c45)cc3)ccc2c1 ; c1ccc(-c2ccc3c(sc4ccccc43)c2N(c2cc3ccccc3c3ccccc23)c2cccc3sc4ccccc4c23)cc1]

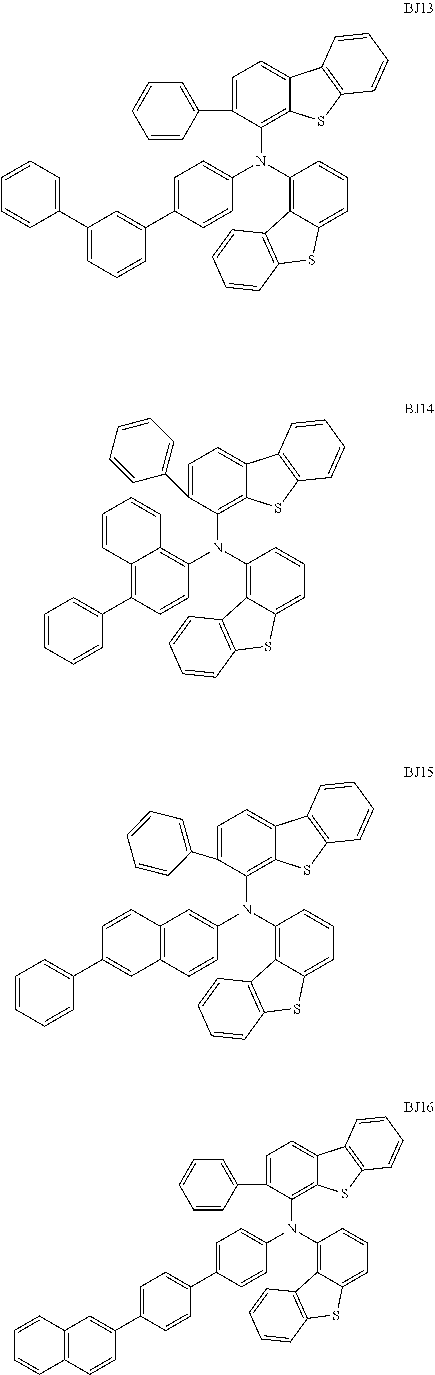 [SMILES: c1ccc(-c2cccc(-c3ccc(N(c4c(-c5ccccc5)ccc5c4sc4ccccc45)c4cccc5sc6ccccc6c45)cc3)c2)cc1 ; c1ccc(-c2ccc3c(sc4ccccc43)c2N(c2ccc(-c3ccccc3)c3ccccc23)c2cccc3sc4ccccc4c23)cc1 ; c1ccc(-c2ccc3cc(N(c4c(-c5ccccc5)ccc5c4sc4ccccc45)c4cccc5sc6ccccc6c45)ccc3c2)cc1 ; c1ccc(-c2ccc3c(sc4ccccc43)c2N(c2ccc(-c3ccc(-c4ccc5ccccc5c4)cc3)cc2)c2cccc3sc4ccccc4c23)cc1]

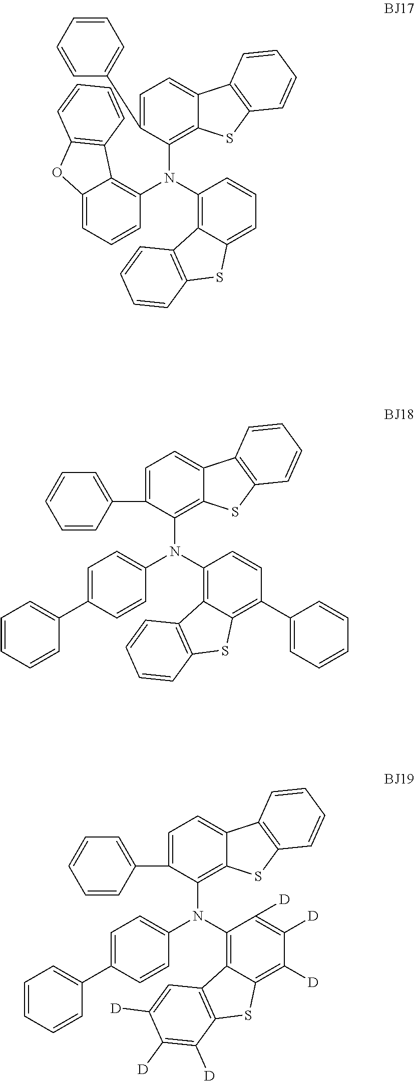 [SMILES: c1ccc(-c2ccc3c(sc4ccccc43)c2N(c2cccc3oc4ccccc4c23)c2cccc3sc4ccccc4c23)cc1 ; c1ccc(-c2ccc(N(c3c(-c4ccccc4)ccc4c3sc3ccccc34)c3ccc(-c4ccccc4)c4sc5ccccc5c34)cc2)cc1 ; [2H]c1cc2c(sc3c([2H])c([2H])c([2H])c(N(c4ccc(-c5ccccc5)cc4)c4c(-c5ccccc5)ccc5c4sc4ccccc45)c32)c([2H])c1[2H]]

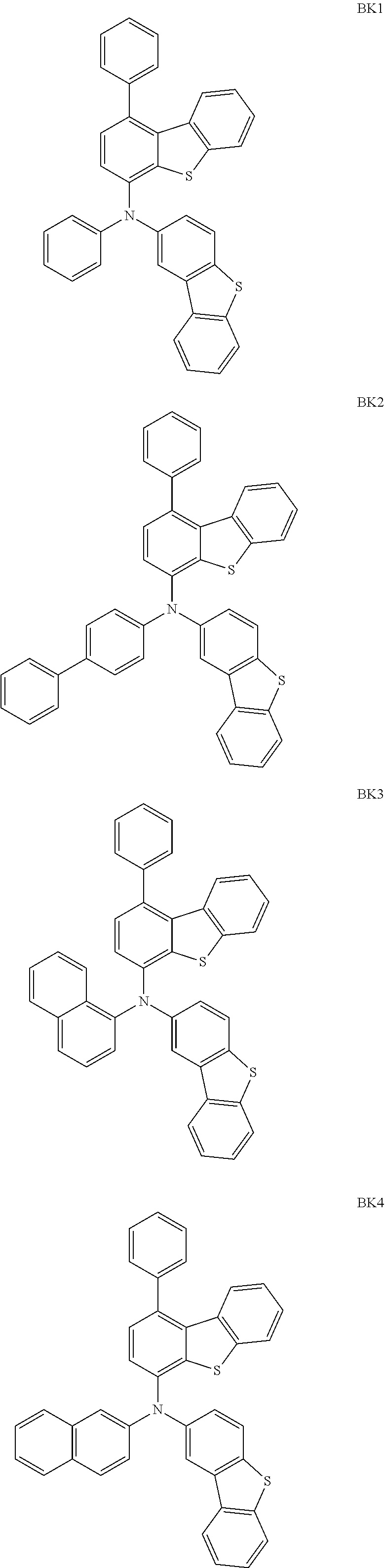

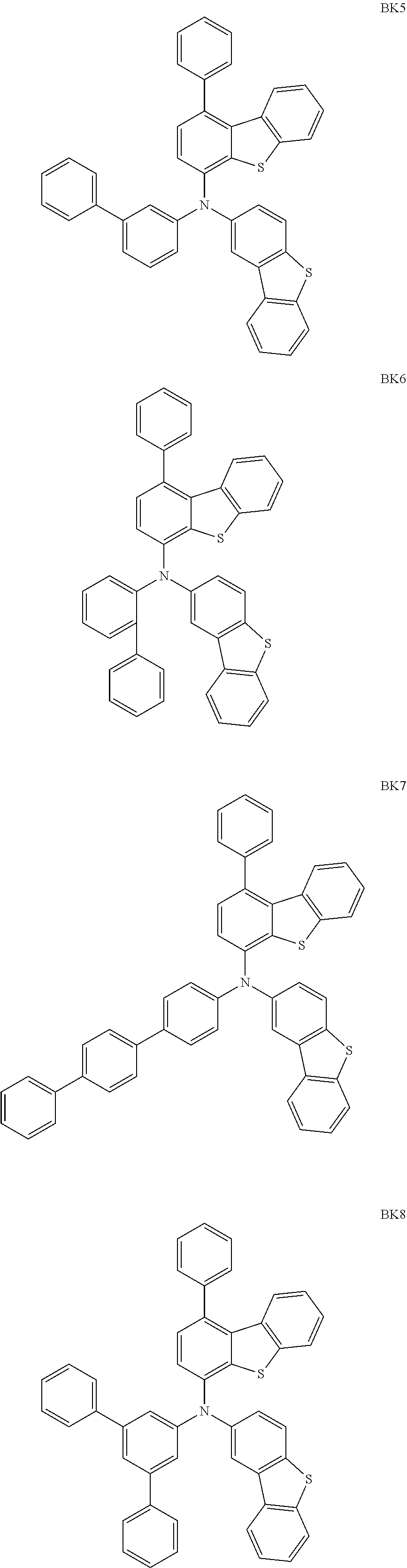 [SMILES: c1ccc(-c2cccc(N(c3ccc4sc5ccccc5c4c3)c3ccc(-c4ccccc4)c4c3sc3ccccc34)c2)cc1 ; c1ccc(-c2ccccc2N(c2ccc3sc4ccccc4c3c2)c2ccc(-c3ccccc3)c3c2sc2ccccc23)cc1 ; c1ccc(-c2ccc(-c3ccc(N(c4ccc5sc6ccccc6c5c4)c4ccc(-c5ccccc5)c5c4sc4ccccc45)cc3)cc2)cc1 ; c1ccc(-c2cc(-c3ccccc3)cc(N(c3ccc4sc5ccccc5c4c3)c3ccc(-c4ccccc4)c4c3sc3ccccc34)c2)cc1]

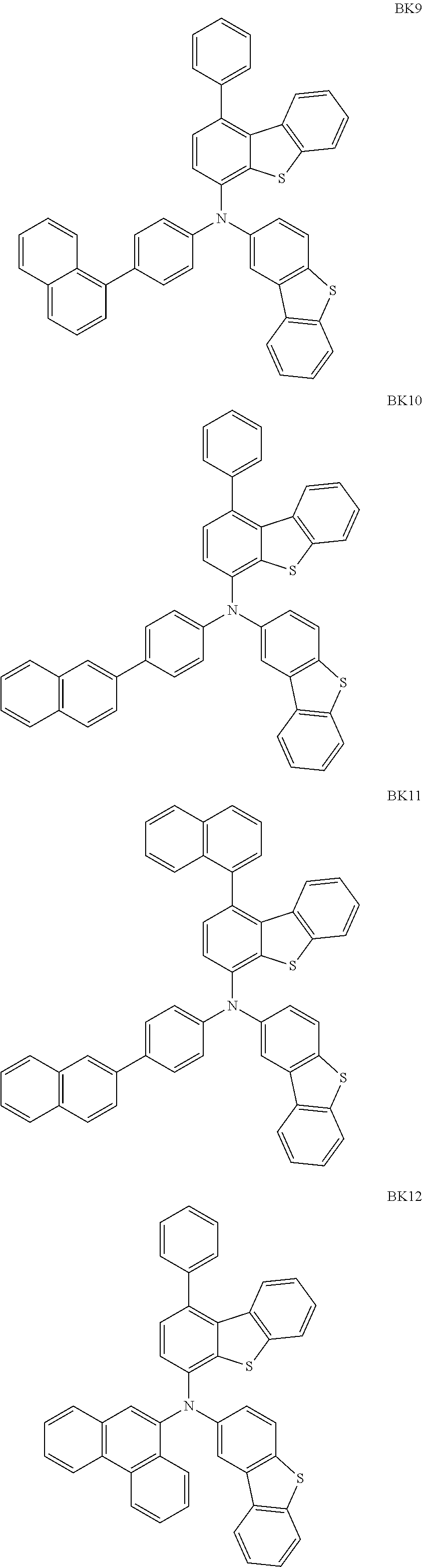 [SMILES: c1ccc(-c2ccc(N(c3ccc(-c4cccc5ccccc45)cc3)c3ccc4sc5ccccc5c4c3)c3sc4ccccc4c23)cc1 ; c1ccc(-c2ccc(N(c3ccc(-c4ccc5ccccc5c4)cc3)c3ccc4sc5ccccc5c4c3)c3sc4ccccc4c23)cc1 ; c1ccc2cc(-c3ccc(N(c4ccc5sc6ccccc6c5c4)c4ccc(-c5cccc6ccccc56)c5c4sc4ccccc45)cc3)ccc2c1 ; c1ccc(-c2ccc(N(c3ccc4sc5ccccc5c4c3)c3cc4ccccc4c4ccccc34)c3sc4ccccc4c23)cc1]

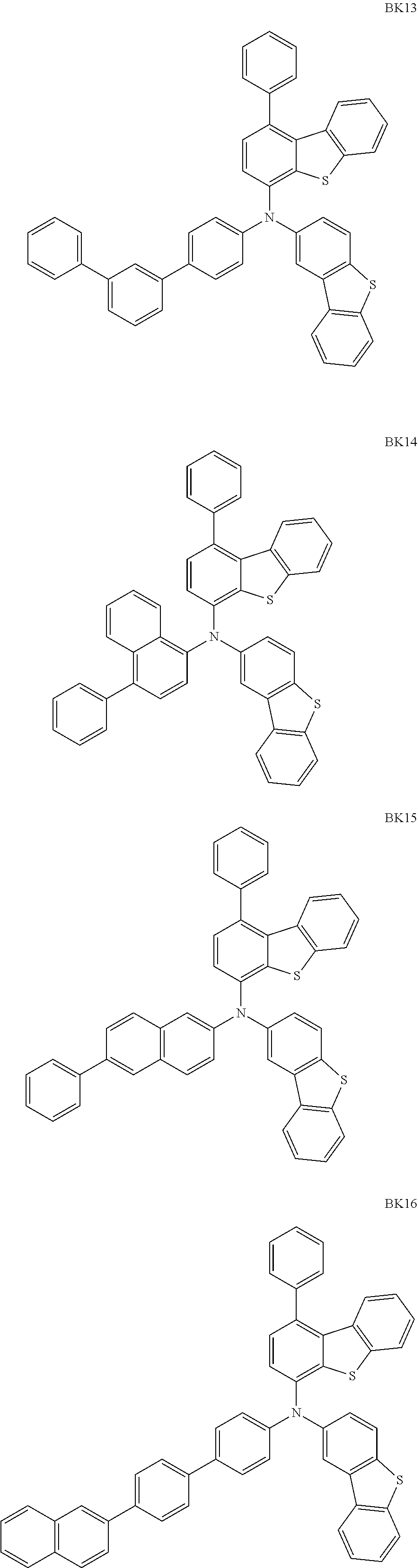 [SMILES: c1ccc(-c2cccc(-c3ccc(N(c4ccc5sc6ccccc6c5c4)c4ccc(-c5ccccc5)c5c4sc4ccccc45)cc3)c2)cc1 ; c1ccc(-c2ccc(N(c3ccc4sc5ccccc5c4c3)c3ccc(-c4ccccc4)c4c3sc3ccccc34)c3ccccc23)cc1 ; c1ccc(-c2ccc3cc(N(c4ccc5sc6ccccc6c5c4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc4sc5ccccc5c4c3)c3sc4ccccc4c23)cc1]

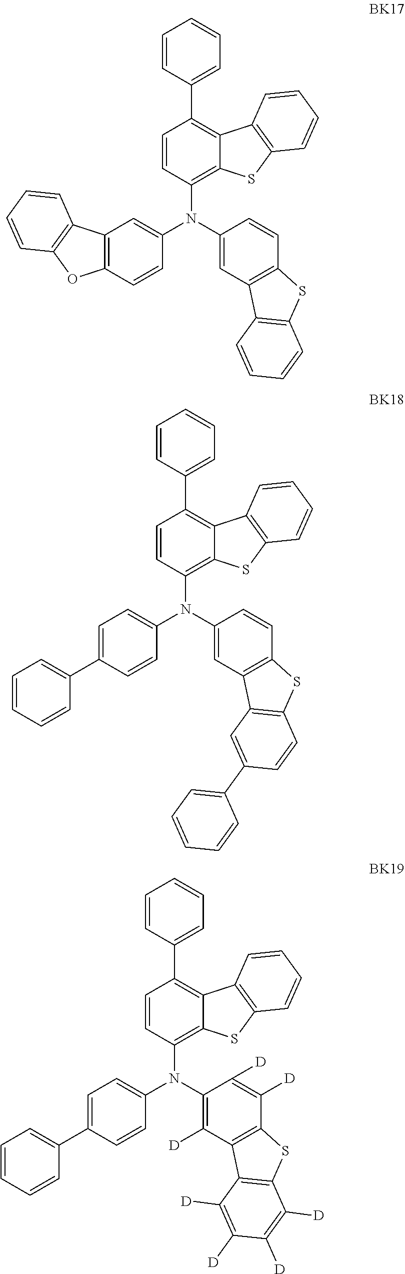

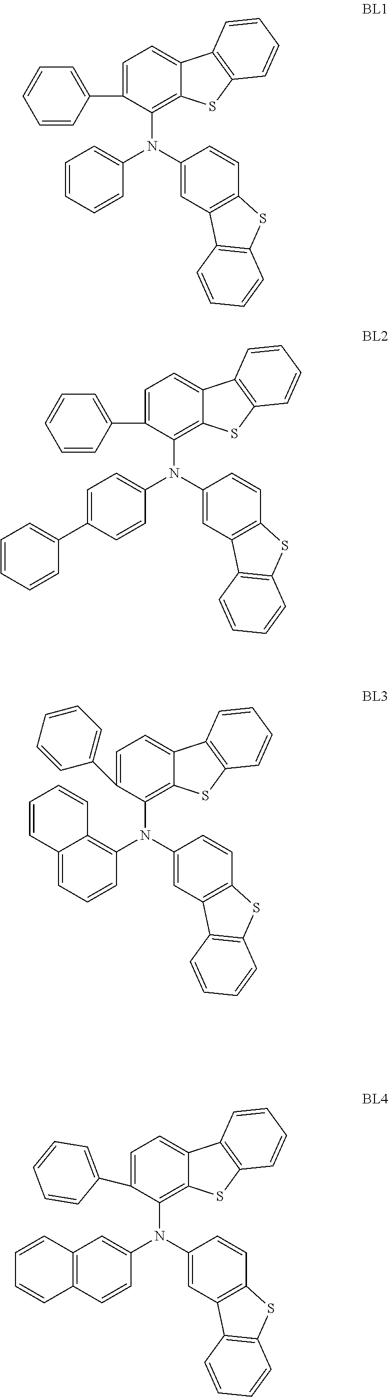

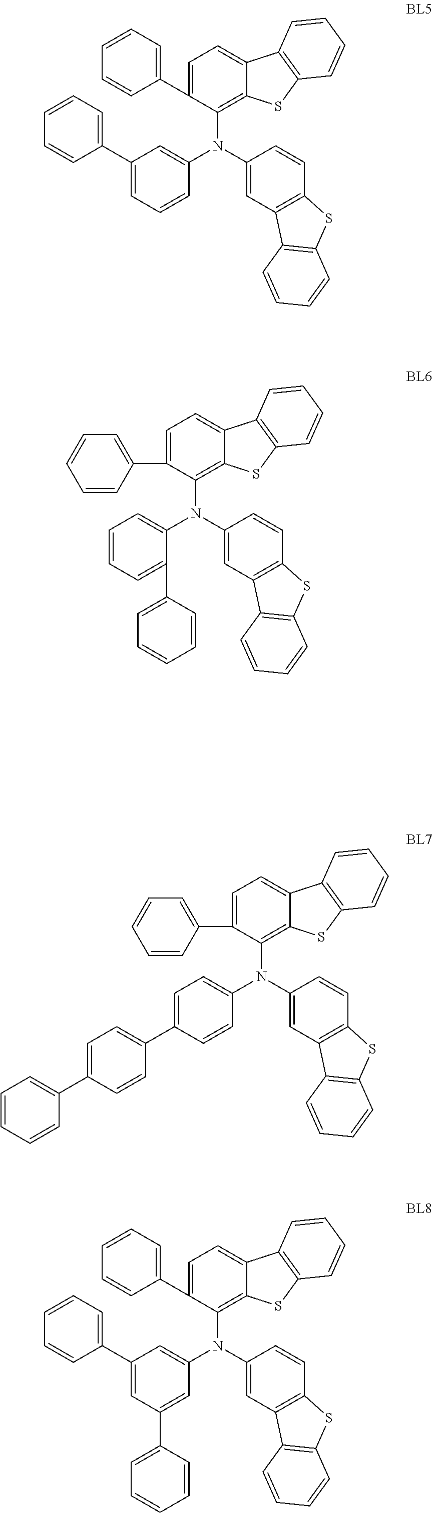 [SMILES: c1ccc(-c2cccc(N(c3ccc4sc5ccccc5c4c3)c3c(-c4ccccc4)ccc4c3sc3ccccc34)c2)cc1 ; c1ccc(-c2ccccc2N(c2ccc3sc4ccccc4c3c2)c2c(-c3ccccc3)ccc3c2sc2ccccc23)cc1 ; c1ccc(-c2ccc(-c3ccc(N(c4ccc5sc6ccccc6c5c4)c4c(-c5ccccc5)ccc5c4sc4ccccc45)cc3)cc2)cc1 ; c1ccc(-c2cc(-c3ccccc3)cc(N(c3ccc4sc5ccccc5c4c3)c3c(-c4ccccc4)ccc4c3sc3ccccc34)c2)cc1]

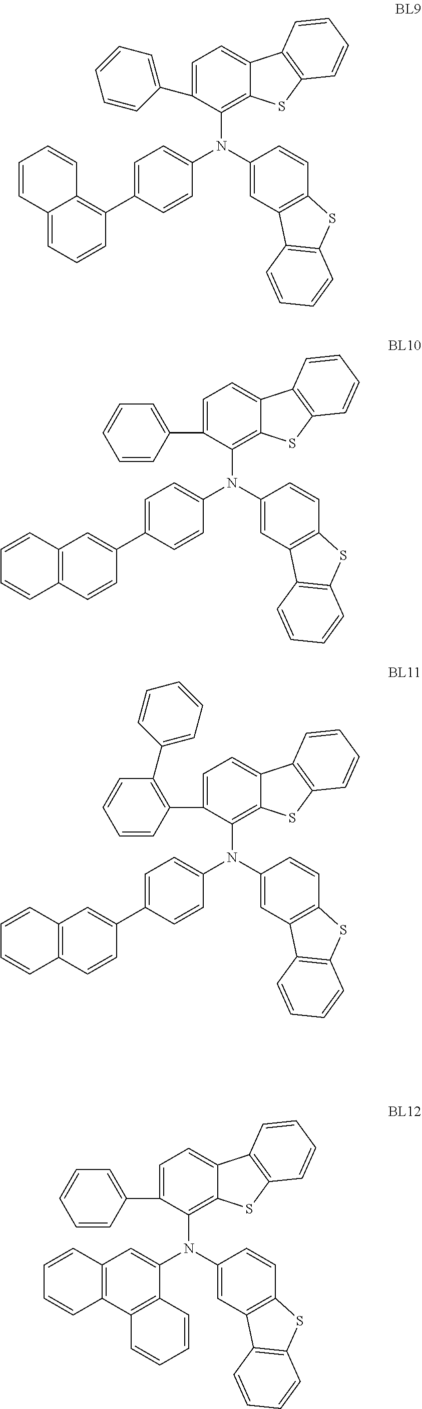 [SMILES: c1ccc(-c2ccc3c(sc4ccccc43)c2N(c2ccc(-c3cccc4ccccc34)cc2)c2ccc3sc4ccccc4c3c2)cc1 ; c1ccc(-c2ccc3c(sc4ccccc43)c2N(c2ccc(-c3ccc4ccccc4c3)cc2)c2ccc3sc4ccccc4c3c2)cc1 ; c1ccc(-c2ccccc2-c2ccc3c(sc4ccccc43)c2N(c2ccc(-c3ccc4ccccc4c3)cc2)c2ccc3sc4ccccc4c3c2)cc1 ; c1ccc(-c2ccc3c(sc4ccccc43)c2N(c2ccc3sc4ccccc4c3c2)c2cc3ccccc3c3ccccc23)cc1]

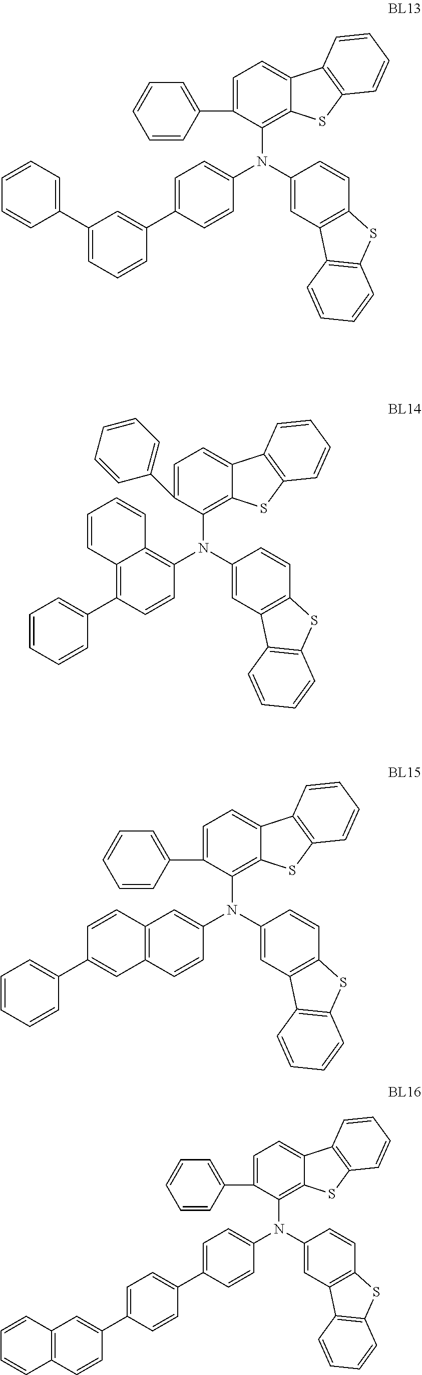 [SMILES: c1ccc(-c2cccc(-c3ccc(N(c4ccc5sc6ccccc6c5c4)c4c(-c5ccccc5)ccc5c4sc4ccccc45)cc3)c2)cc1 ; c1ccc(-c2ccc3c(sc4ccccc43)c2N(c2ccc3sc4ccccc4c3c2)c2ccc(-c3ccccc3)c3ccccc23)cc1 ; c1ccc(-c2ccc3cc(N(c4ccc5sc6ccccc6c5c4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc3sc4ccccc4c3c2)cc1]

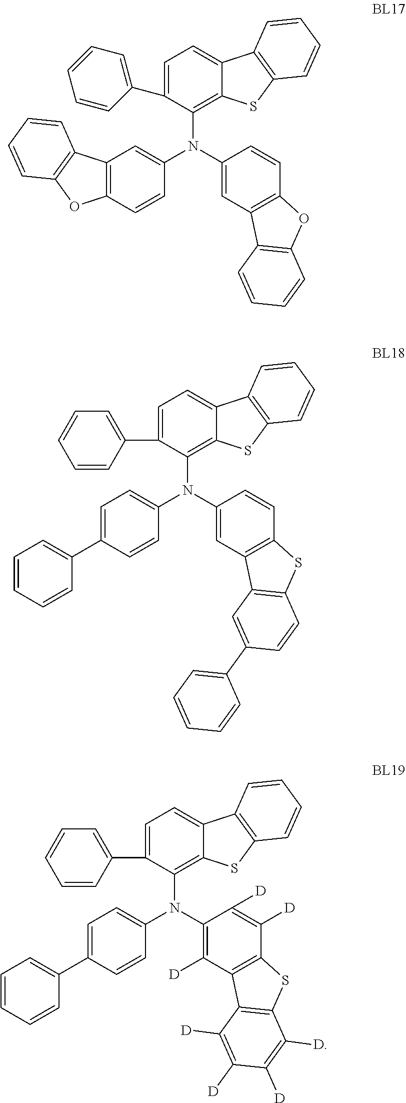

14. An amine compound represented by Formula 1 below: 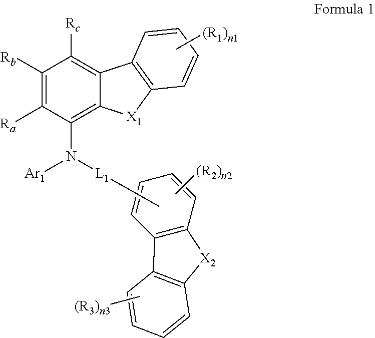
wherein, in Formula 1 above,
X₁ and X₂ are each independently O or S,
Ar₁ is a substituted or unsubstituted aryl group having 6 to 40 ring-forming carbon atoms, or a substituted or unsubstituted heteroaryl group having 2 to 40 ring-forming carbon atoms, and
Rₐ to R_{c} are each independently a hydrogen atom, a deuterium atom, a substituted or unsubstituted aryl group having 6 to 40 ring-forming carbon atoms, or a substituted or unsubstituted heteroaryl group having 2 to 40 ring-forming carbon atoms,
at least one selected from Rₐ and R_{c} is a substituted or unsubstituted aryl group having 6 to 40 ring-forming carbon atoms, or a substituted or unsubstituted heteroaryl group having 2 to 40 ring-forming carbon atoms,
R₁ to R₃ are each independently a hydrogen atom, a deuterium atom, a halogen atom, a substituted or unsubstituted alkyl group having 1 to 20 carbon atoms, a substituted or unsubstituted aryl group having 6 to 40 ring-forming carbon atoms, or a substituted or unsubstituted heteroaryl group having 2 to 40 ring-forming carbon atoms,
L₁ is a direct linkage, a substituted or unsubstituted arylene group having 6 to 40 ring-forming carbon atoms, or a substituted or unsubstituted heteroarylene group having 2 to 40 ring-forming carbon atoms,
the case where each of Rₐ to R_{c} comprises a carbazole group is excluded,
the case where each of Ar₁, Rₐ to R_{c}, L₁, and R₁ to R₃ comprises a fluorenyl group, a benzonaphthofuranyl group, a benzonaphthothiophenyl group, and a benzobisbenzofuranyl group is excluded,
n1 and n3 are each independently an integer of 0 to 4, and
n2 is an integer of 0 to 3.

15. The amine compound of claim 14, wherein the amine compound represented by Formula 1 above is represented by any one selected from among Formula 2-1 to Formula 2-3 below: 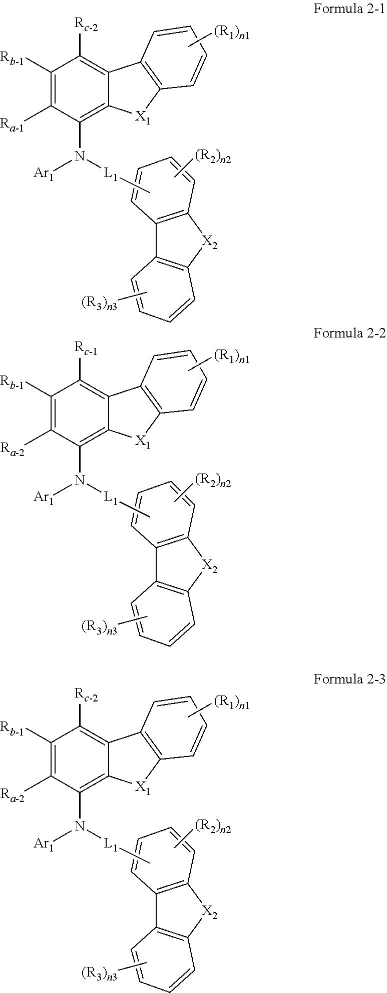
wherein, in Formula 2-1 to Formula 2-3 above,
Rₐ₋₁, R_{b-1}, and R_{c-1} are each independently a hydrogen atom or a deuterium atom,
Rₐ₋₂ and R_{c-2} are each independently a substituted or unsubstituted aryl group having 6 to 40 ring-forming carbon atoms, or a substituted or unsubstituted heteroaryl group having 2 to 40 ring-forming carbon atoms,
the case where each of Rₐ to R_{c} comprises a carbazole group is excluded,
the case where each of Rₐ₋₂ and R_{c-2} is a fluorenyl group, a benzonaphthofuranyl group, a benzonaphthothiophenyl group, or a benzobisbenzofuranyl group is excluded, and
X₁, X₂, Ar₁, R₁ to R₃, L₁, n1, n2, and n3 are the same as defined with respect to Formula 1 above.

16. The amine compound of claim 14, wherein the amine compound represented by Formula 1 above is represented by any one selected from among Formula 3-1 to Formula 3-4 below: 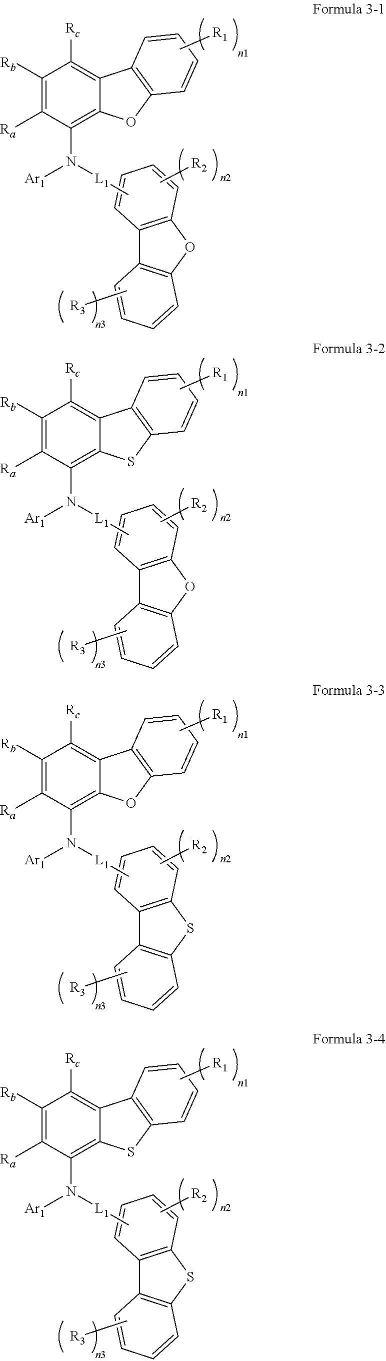
wherein, in Formula 3-1 to Formula 3-4 above,
Ar₁, Rₐ to R_{c}, R₁ to R₃, L₁, n1, n2, and n3 are the same as defined with respect to Formula 1 above.

17. The amine compound of claim 14, wherein the amine compound represented by Formula 1 above is represented by any one selected from among Formula 4-1 to Formula 4-4 below: 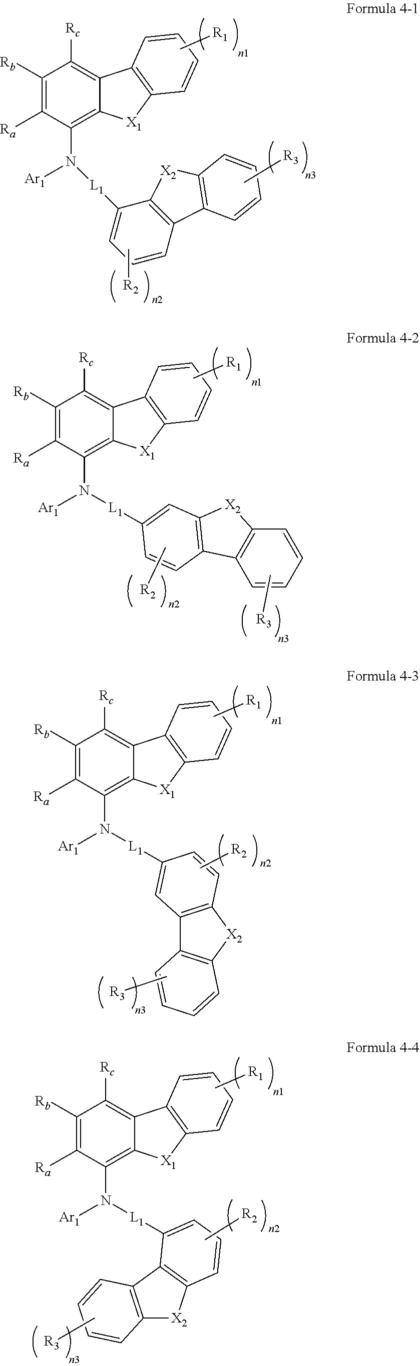
wherein, in Formula 4-1 to Formula 4-4 above,
X₁, X₂, Ar₁, Rₐ to R_{c}, R₁ to R₃, L₁, n1, n2, and n3 are the same as defined with respect to Formula 1 above.

18. The amine compound of claim 14, wherein, in Formula 1 above,
at least one selected from Rₐ and R_{c} is a substituted or unsubstituted phenyl group, a substituted or unsubstituted naphthyl group, a substituted or unsubstituted biphenyl group, a substituted or unsubstituted phenanthrenyl group, a substituted or unsubstituted dibenzofuranyl group, or a substituted or unsubstituted dibenzothiophenyl group.

19. The amine compound of claim 14, wherein, in Formula 1 above,
Ar₁ is a substituted or unsubstituted phenyl group, a substituted or unsubstituted biphenyl group, a substituted or unsubstituted terphenyl group, a substituted or unsubstituted naphthyl group, a substituted or unsubstituted phenanthrene group, a substituted or unsubstituted dibenzofuranyl group, or a substituted or unsubstituted dibenzothiophenyl group.

20. The amine compound of claim 14, wherein the amine compound represented by Formula 1 above is represented by any one selected from among compounds in Compound Group 1 below:
Compound Group 1 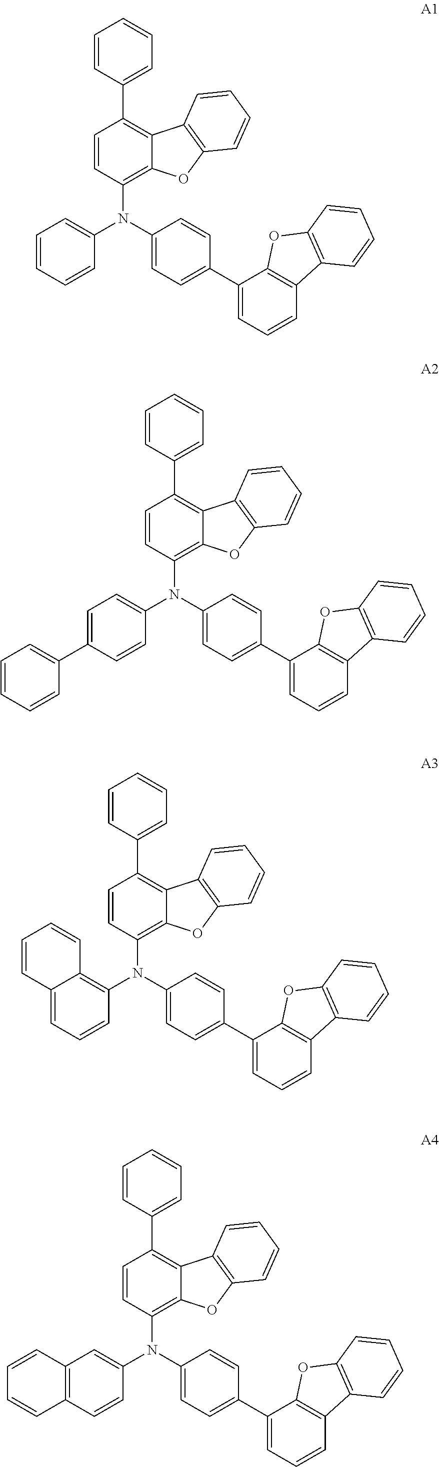 [SMILES: c1ccc(-c2ccc(N(c3ccccc3)c3ccc(-c4cccc5c4oc4ccccc45)cc3)c3oc4ccccc4c23)cc1 ; c1ccc(-c2ccc(N(c3ccc(-c4cccc5c4oc4ccccc45)cc3)c3ccc(-c4ccccc4)c4c3oc3ccccc34)cc2)cc1 ; c1ccc(-c2ccc(N(c3ccc(-c4cccc5c4oc4ccccc45)cc3)c3cccc4ccccc34)c3oc4ccccc4c23)cc1 ; c1ccc(-c2ccc(N(c3ccc(-c4cccc5c4oc4ccccc45)cc3)c3ccc4ccccc4c3)c3oc4ccccc4c23)cc1] 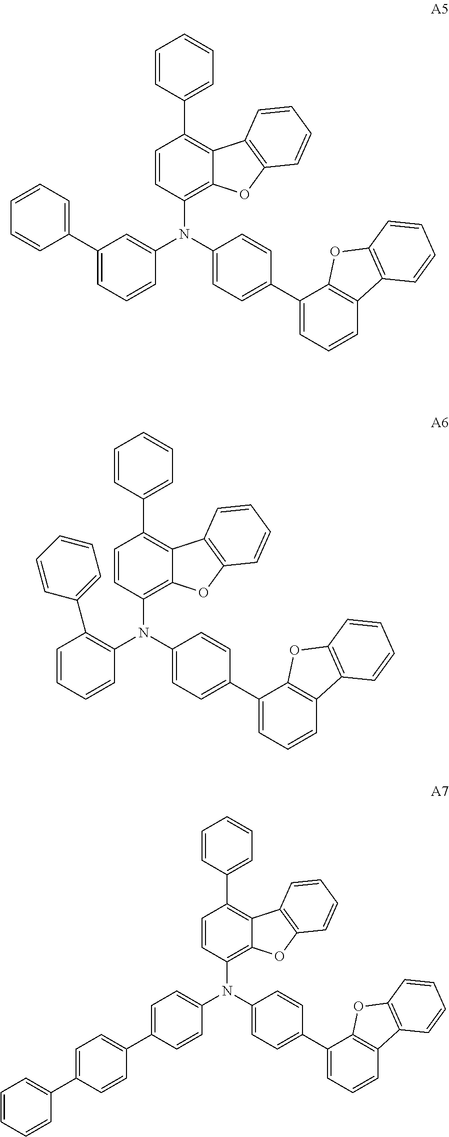 [SMILES: c1ccc(-c2cccc(N(c3ccc(-c4cccc5c4oc4ccccc45)cc3)c3ccc(-c4ccccc4)c4c3oc3ccccc34)c2)cc1 ; c1ccc(-c2ccccc2N(c2ccc(-c3cccc4c3oc3ccccc34)cc2)c2ccc(-c3ccccc3)c3c2oc2ccccc23)cc1 ; c1ccc(-c2ccc(-c3ccc(N(c4ccc(-c5cccc6c5oc5ccccc56)cc4)c4ccc(-c5ccccc5)c5c4oc4ccccc45)cc3)cc2)cc1] 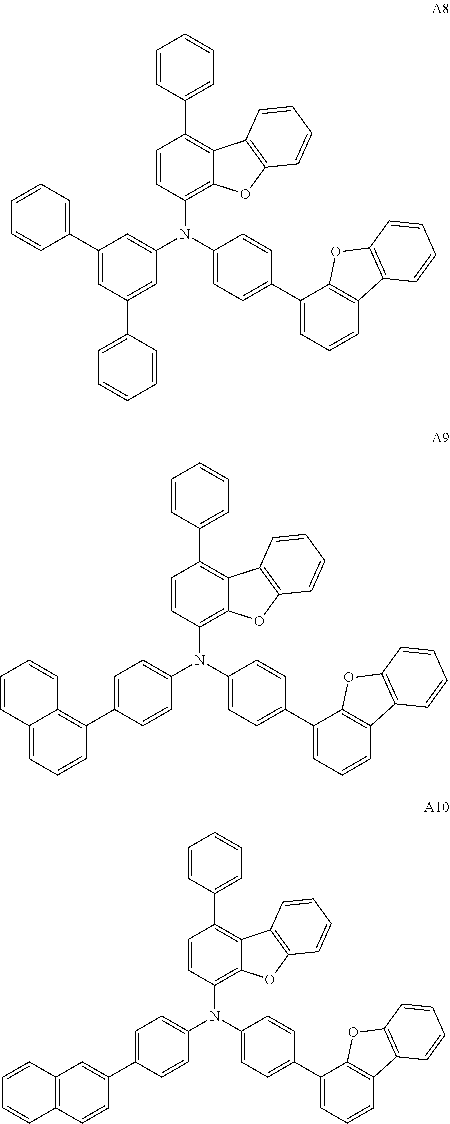 [SMILES: c1ccc(-c2cc(-c3ccccc3)cc(N(c3ccc(-c4cccc5c4oc4ccccc45)cc3)c3ccc(-c4ccccc4)c4c3oc3ccccc34)c2)cc1 ; c1ccc(-c2ccc(N(c3ccc(-c4cccc5ccccc45)cc3)c3ccc(-c4cccc5c4oc4ccccc45)cc3)c3oc4ccccc4c23)cc1 ; c1ccc(-c2ccc(N(c3ccc(-c4ccc5ccccc5c4)cc3)c3ccc(-c4cccc5c4oc4ccccc45)cc3)c3oc4ccccc4c23)cc1] 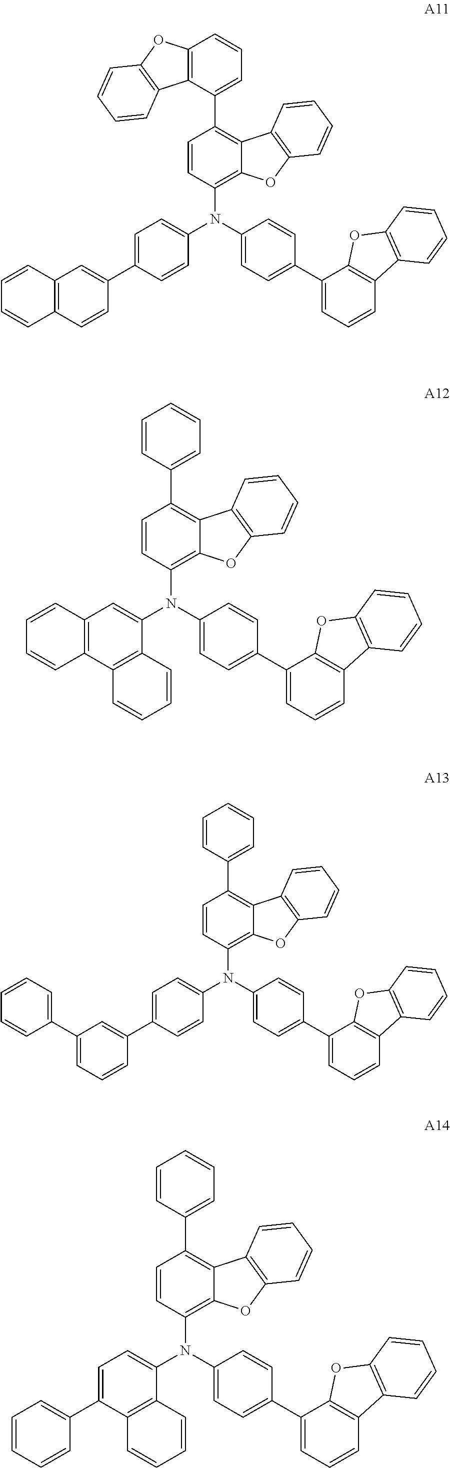 [SMILES: c1ccc2cc(-c3ccc(N(c4ccc(-c5cccc6c5oc5ccccc56)cc4)c4ccc(-c5cccc6oc7ccccc7c56)c5c4oc4ccccc45)cc3)ccc2c1 ; c1ccc(-c2ccc(N(c3ccc(-c4cccc5c4oc4ccccc45)cc3)c3cc4ccccc4c4ccccc34)c3oc4ccccc4c23)cc1 ; c1ccc(-c2cccc(-c3ccc(N(c4ccc(-c5cccc6c5oc5ccccc56)cc4)c4ccc(-c5ccccc5)c5c4oc4ccccc45)cc3)c2)cc1 ; c1ccc(-c2ccc(N(c3ccc(-c4cccc5c4oc4ccccc45)cc3)c3ccc(-c4ccccc4)c4c3oc3ccccc34)c3ccccc23)cc1] 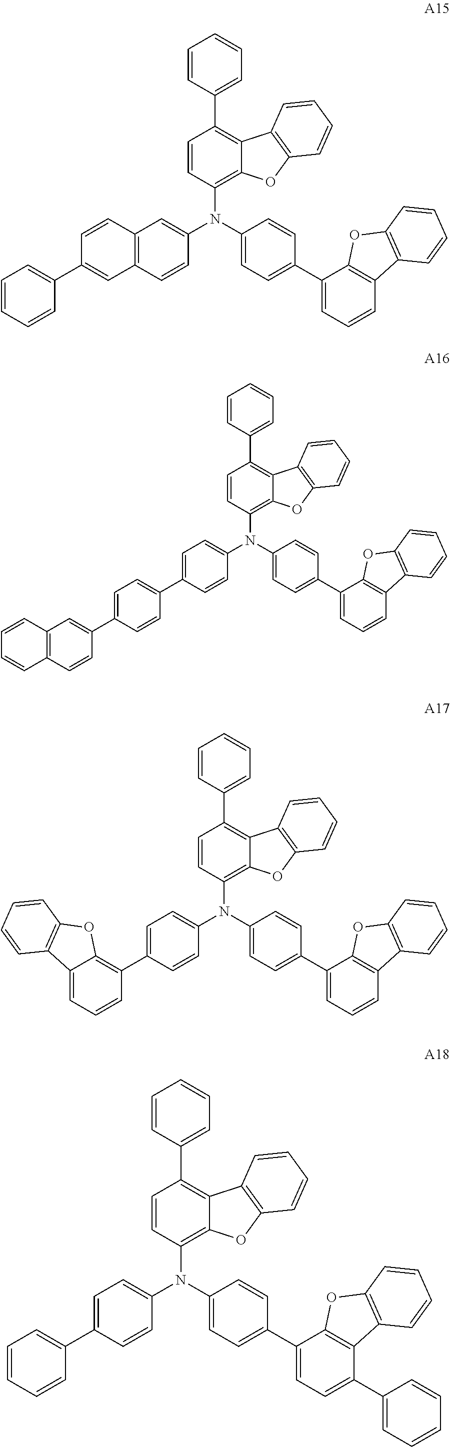 [SMILES: c1ccc(-c2ccc3cc(N(c4ccc(-c5cccc6c5oc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4cccc5c4oc4ccccc45)cc3)c3oc4ccccc4c23)cc1 ; c1ccc(-c2ccc(N(c3ccc(-c4cccc5c4oc4ccccc45)cc3)c3ccc(-c4cccc5c4oc4ccccc45)cc3)c3oc4ccccc4c23)cc1 ; c1ccc(-c2ccc(N(c3ccc(-c4ccc(-c5ccccc5)c5c4oc4ccccc45)cc3)c3ccc(-c4ccccc4)c4c3oc3ccccc34)cc2)cc1] 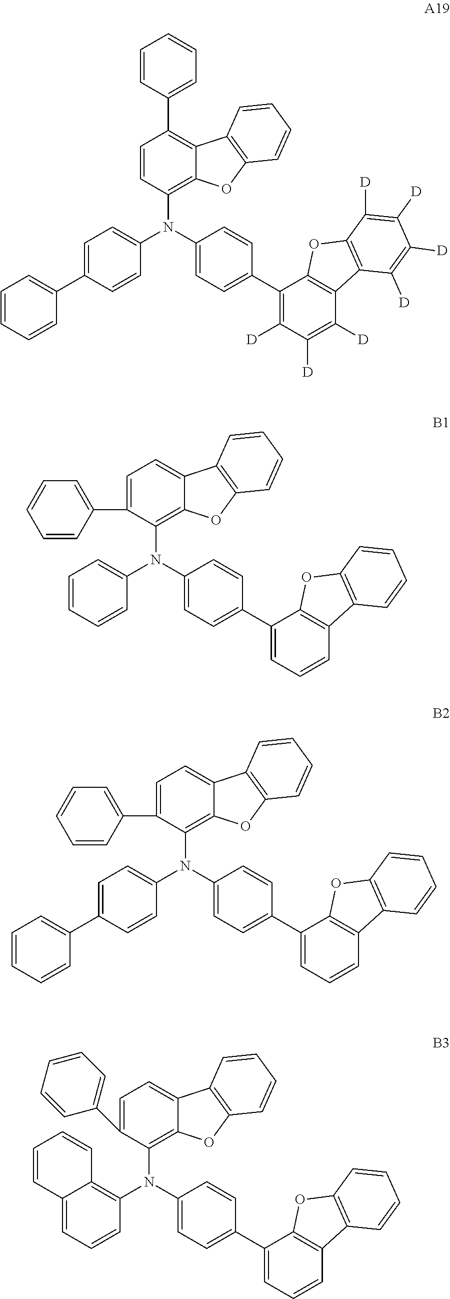 [SMILES: [2H]c1c([2H])c([2H])c2c(oc3c(-c4ccc(N(c5ccc(-c6ccccc6)cc5)c5ccc(-c6ccccc6)c6c5oc5ccccc56)cc4)c([2H])c([2H])c([2H])c32)c1[2H] ; c1ccc(-c2ccc3c(oc4ccccc43)c2N(c2ccccc2)c2ccc(-c3cccc4c3oc3ccccc34)cc2)cc1 ; c1ccc(-c2ccc(N(c3ccc(-c4cccc5c4oc4ccccc45)cc3)c3c(-c4ccccc4)ccc4c3oc3ccccc34)cc2)cc1 ; c1ccc(-c2ccc3c(oc4ccccc43)c2N(c2ccc(-c3cccc4c3oc3ccccc34)cc2)c2cccc3ccccc23)cc1] 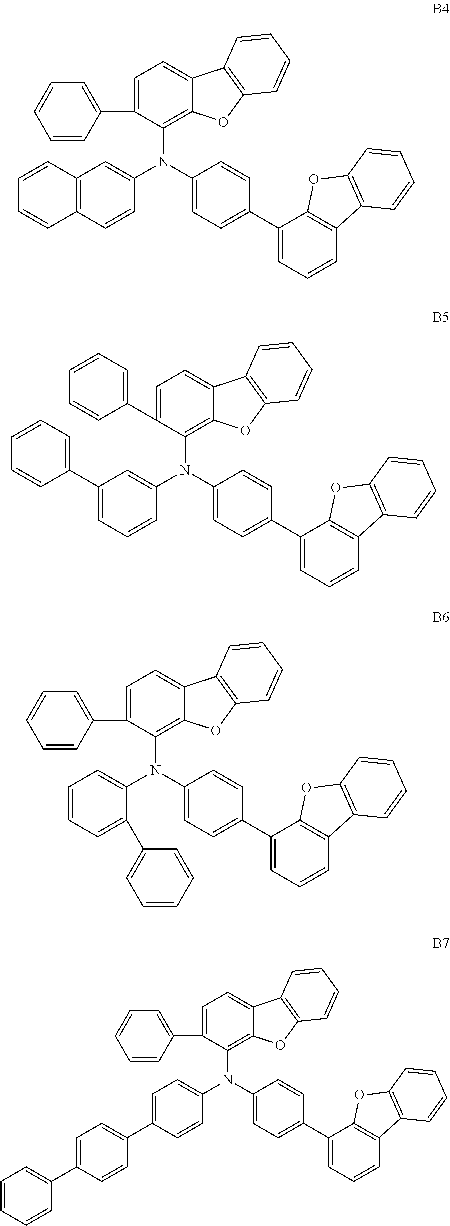 [SMILES: c1ccc(-c2ccc3c(oc4ccccc43)c2N(c2ccc(-c3cccc4c3oc3ccccc34)cc2)c2ccc3ccccc3c2)cc1 ; c1ccc(-c2cccc(N(c3ccc(-c4cccc5c4oc4ccccc45)cc3)c3c(-c4ccccc4)ccc4c3oc3ccccc34)c2)cc1 ; c1ccc(-c2ccccc2N(c2ccc(-c3cccc4c3oc3ccccc34)cc2)c2c(-c3ccccc3)ccc3c2oc2ccccc23)cc1 ; c1ccc(-c2ccc(-c3ccc(N(c4ccc(-c5cccc6c5oc5ccccc56)cc4)c4c(-c5ccccc5)ccc5c4oc4ccccc45)cc3)cc2)cc1] 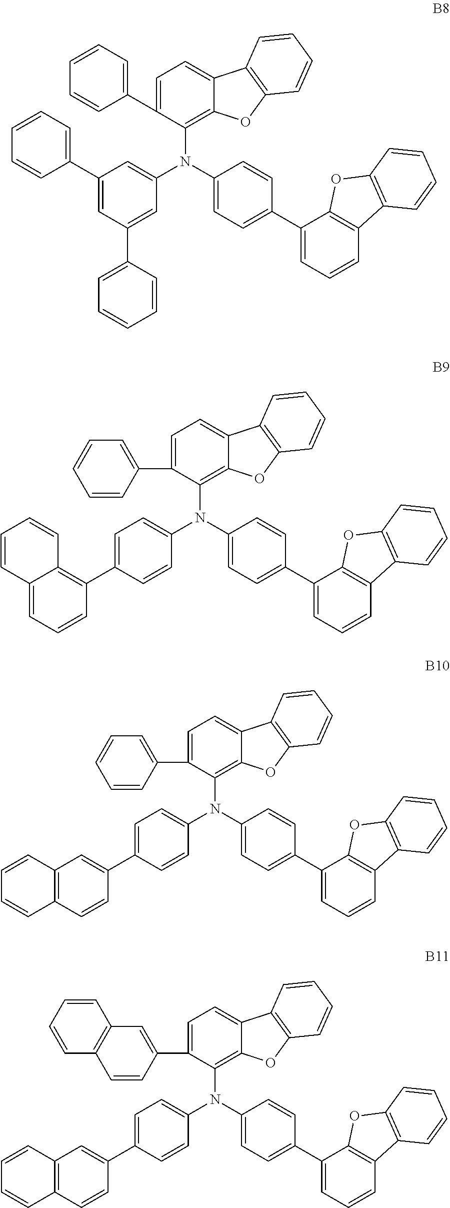 [SMILES: c1ccc(-c2cc(-c3ccccc3)cc(N(c3ccc(-c4cccc5c4oc4ccccc45)cc3)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc(-c3cccc4c3oc3ccccc34)cc2)cc1 ; c1ccc(-c2ccc3c(oc4ccccc43)c2N(c2ccc(-c3ccc4ccccc4c3)cc2)c2ccc(-c3cccc4c3oc3ccccc34)cc2)cc1 ; c1ccc2cc(-c3ccc(N(c4ccc(-c5cccc6c5oc5ccccc56)cc4)c4c(-c5ccc6ccccc6c5)ccc5c4oc4ccccc45)cc3)ccc2c1] 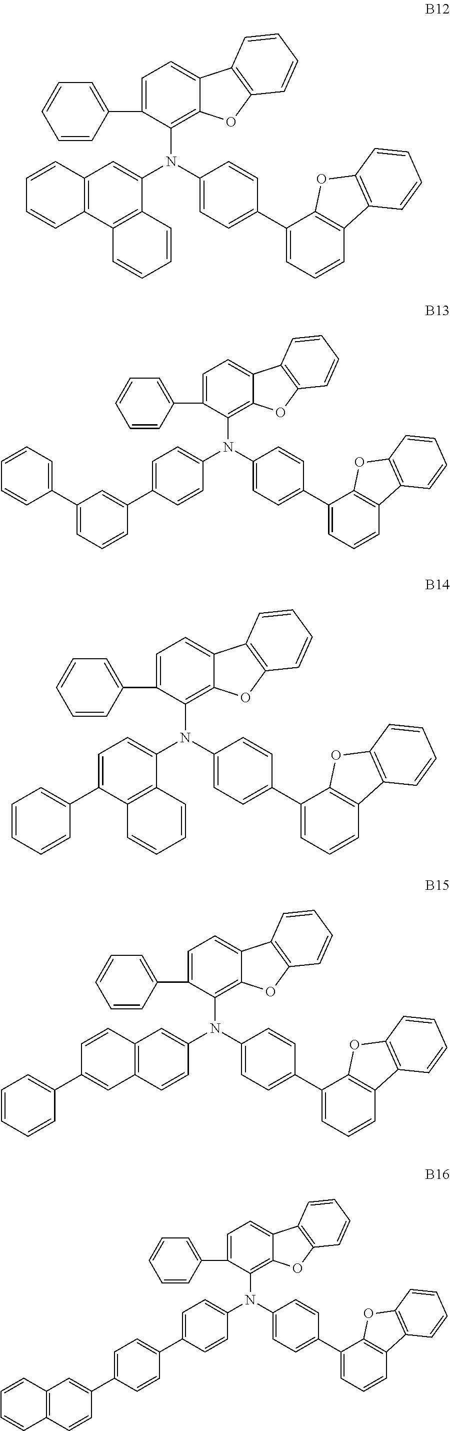 [SMILES: c1ccc(-c2ccc3c(oc4ccccc43)c2N(c2ccc(-c3cccc4c3oc3ccccc34)cc2)c2cc3ccccc3c3ccccc23)cc1 ; c1ccc(-c2cccc(-c3ccc(N(c4ccc(-c5cccc6c5oc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3cccc4c3oc3ccccc34)cc2)c2ccc(-c3ccccc3)c3ccccc23)cc1 ; c1ccc(-c2ccc3cc(N(c4ccc(-c5cccc6c5oc5ccccc56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4c3oc3ccccc34)cc2)cc1] 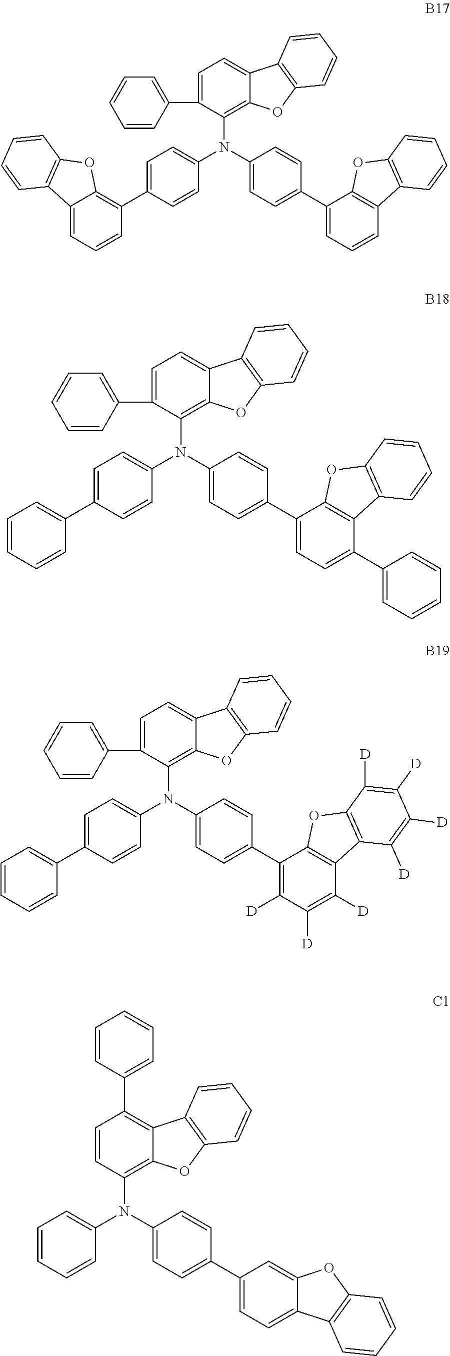 [SMILES: c1ccc(-c2ccc3c(oc4ccccc43)c2N(c2ccc(-c3cccc4c3oc3ccccc34)cc2)c2ccc(-c3cccc4c3oc3ccccc34)cc2)cc1 ; c1ccc(-c2ccc(N(c3ccc(-c4ccc(-c5ccccc5)c5c4oc4ccccc45)cc3)c3c(-c4ccccc4)ccc4c3oc3ccccc34)cc2)cc1 ; [2H]c1c([2H])c([2H])c2c(oc3c(-c4ccc(N(c5ccc(-c6ccccc6)cc5)c5c(-c6ccccc6)ccc6c5oc5ccccc56)cc4)c([2H])c([2H])c([2H])c32)c1[2H] ; c1ccc(-c2ccc(N(c3ccccc3)c3ccc(-c4ccc5c(c4)oc4ccccc45)cc3)c3oc4ccccc4c23)cc1] 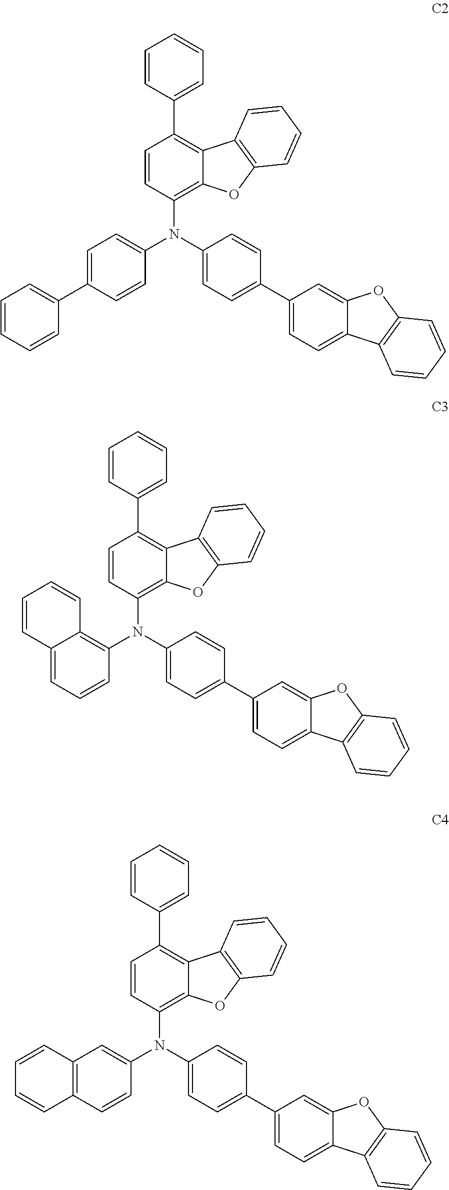 [SMILES: c1ccc(-c2ccc(N(c3ccc(-c4ccc5c(c4)oc4ccccc45)cc3)c3ccc(-c4ccccc4)c4c3oc3ccccc34)cc2)cc1 ; c1ccc(-c2ccc(N(c3ccc(-c4ccc5c(c4)oc4ccccc45)cc3)c3cccc4ccccc34)c3oc4ccccc4c23)cc1 ; c1ccc(-c2ccc(N(c3ccc(-c4ccc5c(c4)oc4ccccc45)cc3)c3ccc4ccccc4c3)c3oc4ccccc4c23)cc1] 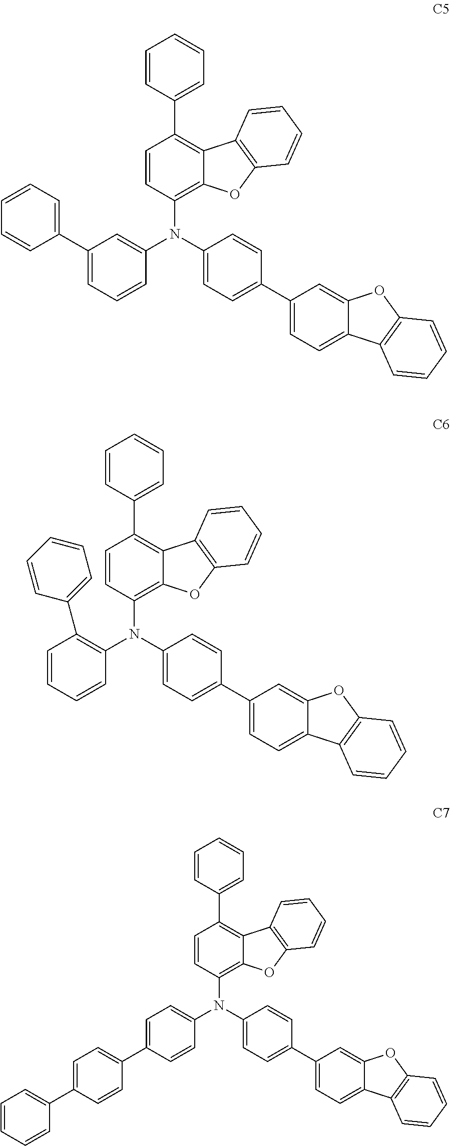 [SMILES: c1ccc(-c2cccc(N(c3ccc(-c4ccc5c(c4)oc4ccccc45)cc3)c3ccc(-c4ccccc4)c4c3oc3ccccc34)c2)cc1 ; c1ccc(-c2ccccc2N(c2ccc(-c3ccc4c(c3)oc3ccccc34)cc2)c2ccc(-c3ccccc3)c3c2oc2ccccc23)cc1 ; c1ccc(-c2ccc(-c3ccc(N(c4ccc(-c5ccc6c(c5)oc5ccccc56)cc4)c4ccc(-c5ccccc5)c5c4oc4ccccc45)cc3)cc2)cc1] 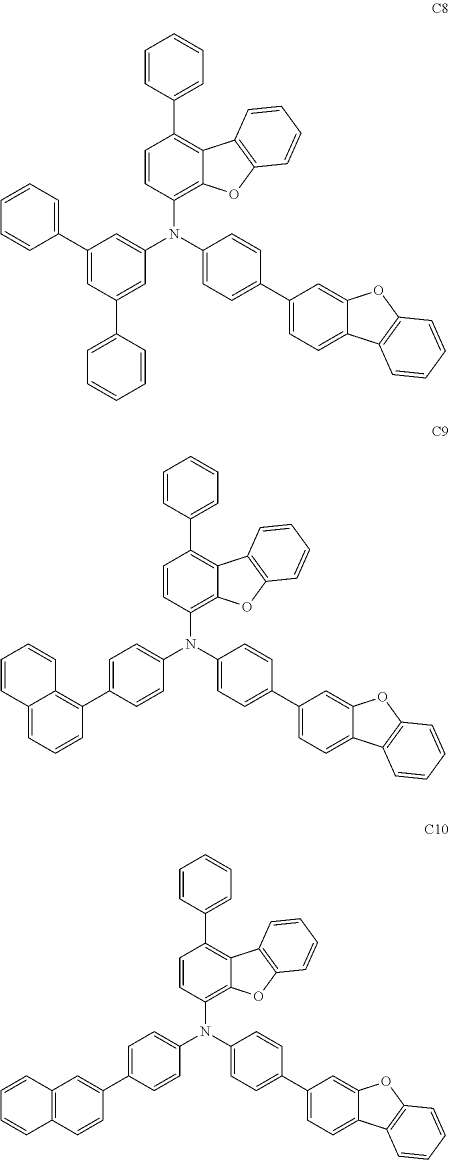 [SMILES: c1ccc(-c2cc(-c3ccccc3)cc(N(c3ccc(-c4ccc5c(c4)oc4ccccc45)cc3)c3ccc(-c4ccccc4)c4c3oc3ccccc34)c2)cc1 ; c1ccc(-c2ccc(N(c3ccc(-c4ccc5c(c4)oc4ccccc45)cc3)c3ccc(-c4cccc5ccccc45)cc3)c3oc4ccccc4c23)cc1 ; c1ccc(-c2ccc(N(c3ccc(-c4ccc5ccccc5c4)cc3)c3ccc(-c4ccc5c(c4)oc4ccccc45)cc3)c3oc4ccccc4c23)cc1] 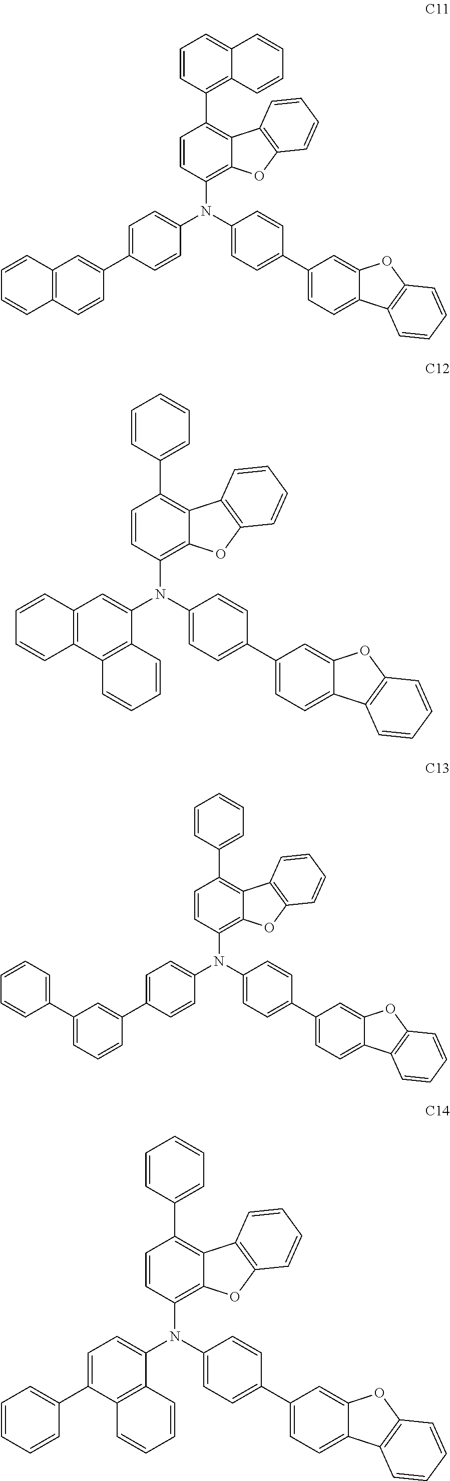 [SMILES: c1ccc2cc(-c3ccc(N(c4ccc(-c5ccc6c(c5)oc5ccccc56)cc4)c4ccc(-c5cccc6ccccc56)c5c4oc4ccccc45)cc3)ccc2c1 ; c1ccc(-c2ccc(N(c3ccc(-c4ccc5c(c4)oc4ccccc45)cc3)c3cc4ccccc4c4ccccc34)c3oc4ccccc4c23)cc1 ; c1ccc(-c2cccc(-c3ccc(N(c4ccc(-c5ccc6c(c5)oc5ccccc56)cc4)c4ccc(-c5ccccc5)c5c4oc4ccccc45)cc3)c2)cc1 ; c1ccc(-c2ccc(N(c3ccc(-c4ccc5c(c4)oc4ccccc45)cc3)c3ccc(-c4ccccc4)c4c3oc3ccccc34)c3ccccc23)cc1] 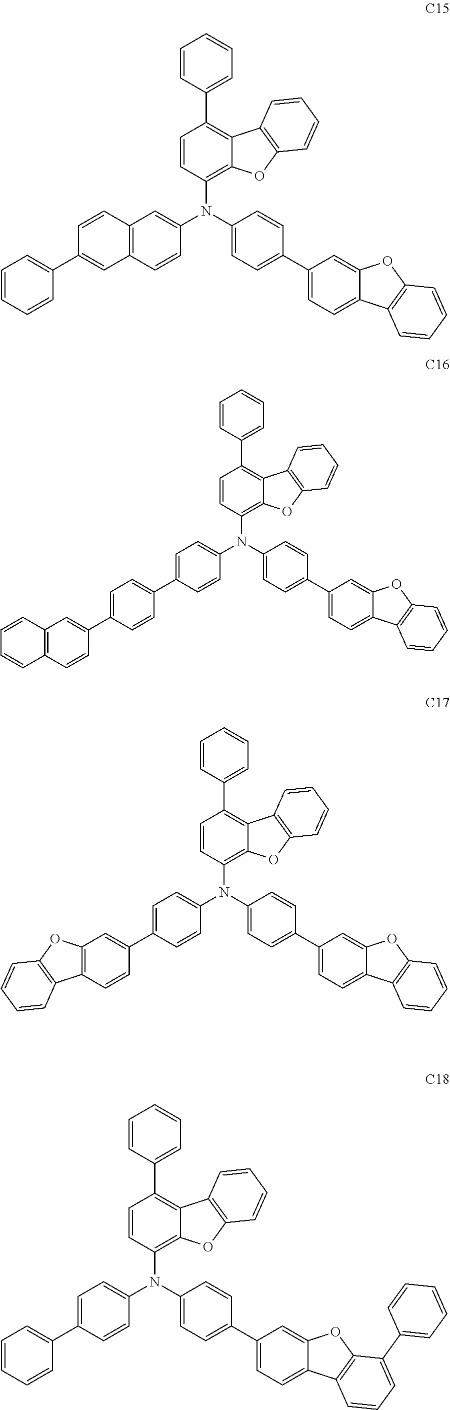 [SMILES: c1ccc(-c2ccc3cc(N(c4ccc(-c5ccc6c(c5)oc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4ccc5c(c4)oc4ccccc45)cc3)c3oc4ccccc4c23)cc1 ; c1ccc(-c2ccc(N(c3ccc(-c4ccc5c(c4)oc4ccccc45)cc3)c3ccc(-c4ccc5c(c4)oc4ccccc45)cc3)c3oc4ccccc4c23)cc1 ; c1ccc(-c2ccc(N(c3ccc(-c4ccc5c(c4)oc4c(-c6ccccc6)cccc45)cc3)c3ccc(-c4ccccc4)c4c3oc3ccccc34)cc2)cc1] 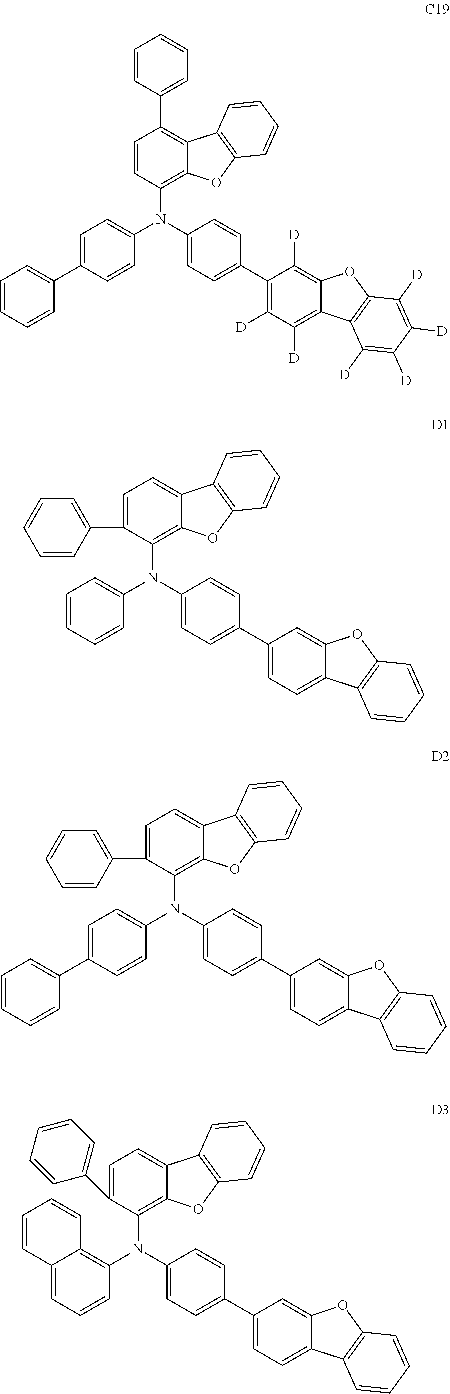 [SMILES: [2H]c1c([2H])c([2H])c2c(oc3c([2H])c(-c4ccc(N(c5ccc(-c6ccccc6)cc5)c5ccc(-c6ccccc6)c6c5oc5ccccc56)cc4)c([2H])c([2H])c32)c1[2H] ; c1ccc(-c2ccc3c(oc4ccccc43)c2N(c2ccccc2)c2ccc(-c3ccc4c(c3)oc3ccccc34)cc2)cc1 ; c1ccc(-c2ccc(N(c3ccc(-c4ccc5c(c4)oc4ccccc45)cc3)c3c(-c4ccccc4)ccc4c3oc3ccccc34)cc2)cc1 ; c1ccc(-c2ccc3c(oc4ccccc43)c2N(c2ccc(-c3ccc4c(c3)oc3ccccc34)cc2)c2cccc3ccccc23)cc1] 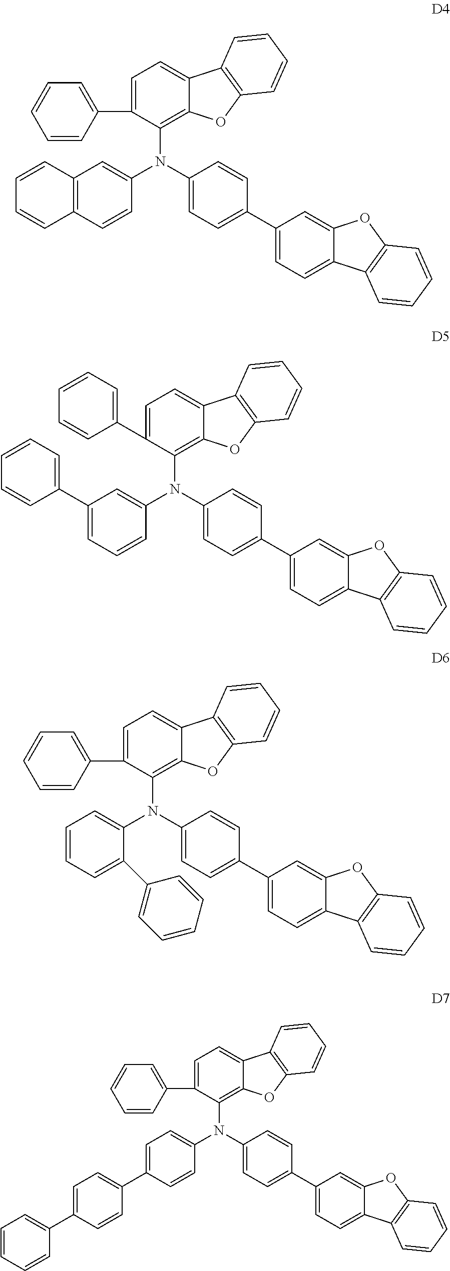 [SMILES: c1ccc(-c2ccc3c(oc4ccccc43)c2N(c2ccc(-c3ccc4c(c3)oc3ccccc34)cc2)c2ccc3ccccc3c2)cc1 ; c1ccc(-c2cccc(N(c3ccc(-c4ccc5c(c4)oc4ccccc45)cc3)c3c(-c4ccccc4)ccc4c3oc3ccccc34)c2)cc1 ; c1ccc(-c2ccccc2N(c2ccc(-c3ccc4c(c3)oc3ccccc34)cc2)c2c(-c3ccccc3)ccc3c2oc2ccccc23)cc1 ; c1ccc(-c2ccc(-c3ccc(N(c4ccc(-c5ccc6c(c5)oc5ccccc56)cc4)c4c(-c5ccccc5)ccc5c4oc4ccccc45)cc3)cc2)cc1] 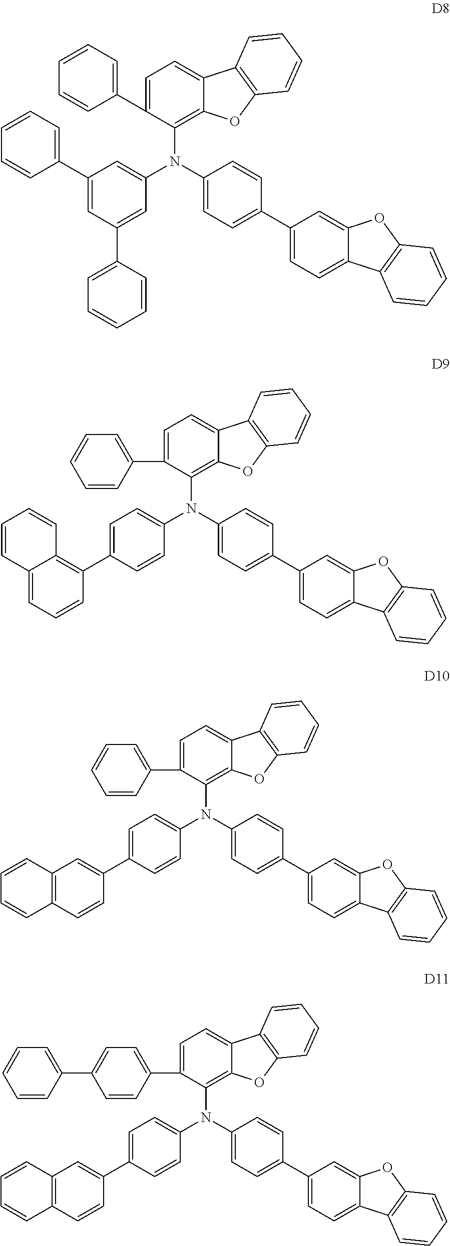 [SMILES: c1ccc(-c2cc(-c3ccccc3)cc(N(c3ccc(-c4ccc5c(c4)oc4ccccc45)cc3)c3c(-c4ccccc4)ccc4c3oc3ccccc34)c2)cc1 ; c1ccc(-c2ccc3c(oc4ccccc43)c2N(c2ccc(-c3ccc4c(c3)oc3ccccc34)cc2)c2ccc(-c3cccc4ccccc34)cc2)cc1 ; c1ccc(-c2ccc3c(oc4ccccc43)c2N(c2ccc(-c3ccc4ccccc4c3)cc2)c2ccc(-c3ccc4c(c3)oc3ccccc34)cc2)cc1 ; c1ccc(-c2ccc(-c3ccc4c(oc5ccccc54)c3N(c3ccc(-c4ccc5ccccc5c4)cc3)c3ccc(-c4ccc5c(c4)oc4ccccc45)cc3)cc2)cc1] 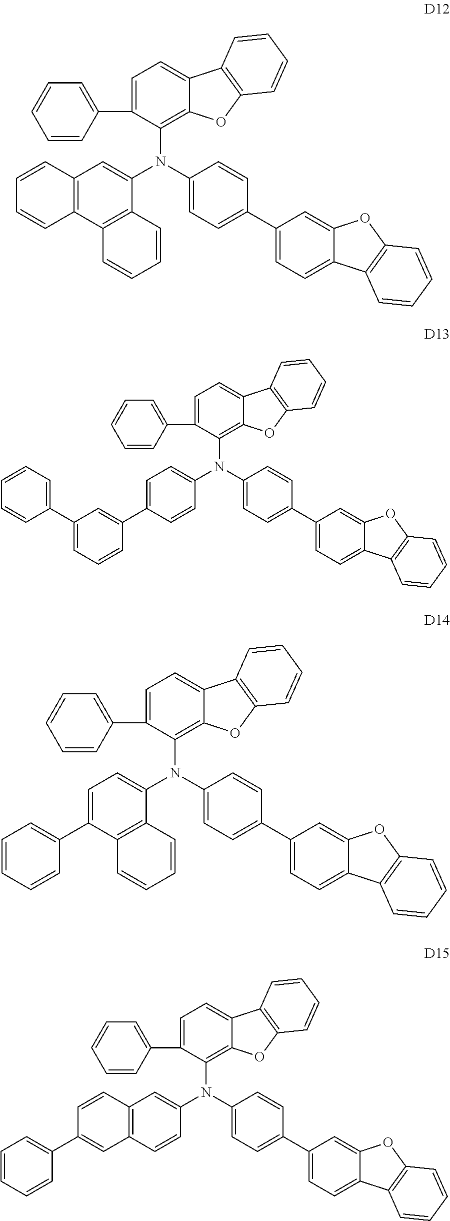 [SMILES: c1ccc(-c2ccc3c(oc4ccccc43)c2N(c2ccc(-c3ccc4c(c3)oc3ccccc34)cc2)c2cc3ccccc3c3ccccc23)cc1 ; c1ccc(-c2cccc(-c3ccc(N(c4ccc(-c5ccc6c(c5)oc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3ccc4c(c3)oc3ccccc34)cc2)c2ccc(-c3ccccc3)c3ccccc23)cc1 ; c1ccc(-c2ccc3cc(N(c4ccc(-c5ccc6c(c5)oc5ccccc56)cc4)c4c(-c5ccccc5)ccc5c4oc4ccccc45)ccc3c2)cc1] 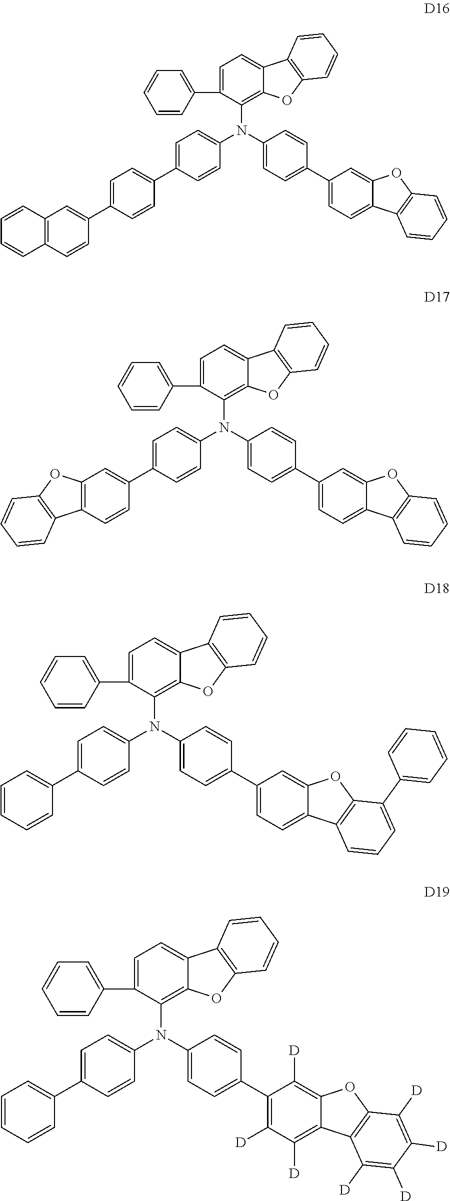 [SMILES: c1ccc(-c2ccc3c(oc4ccccc43)c2N(c2ccc(-c3ccc(-c4ccc5ccccc5c4)cc3)cc2)c2ccc(-c3ccc4c(c3)oc3ccccc34)cc2)cc1 ; c1ccc(-c2ccc3c(oc4ccccc43)c2N(c2ccc(-c3ccc4c(c3)oc3ccccc34)cc2)c2ccc(-c3ccc4c(c3)oc3ccccc34)cc2)cc1 ; c1ccc(-c2ccc(N(c3ccc(-c4ccc5c(c4)oc4c(-c6ccccc6)cccc45)cc3)c3c(-c4ccccc4)ccc4c3oc3ccccc34)cc2)cc1 ; [2H]c1c([2H])c([2H])c2c(oc3c([2H])c(-c4ccc(N(c5ccc(-c6ccccc6)cc5)c5c(-c6ccccc6)ccc6c5oc5ccccc56)cc4)c([2H])c([2H])c32)c1[2H]] 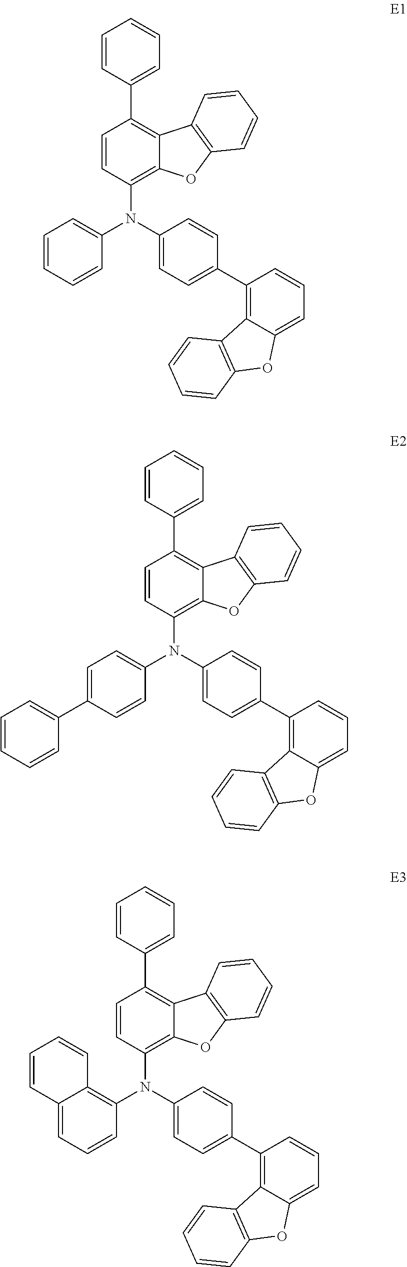 [SMILES: c1ccc(-c2ccc(N(c3ccccc3)c3ccc(-c4cccc5oc6ccccc6c45)cc3)c3oc4ccccc4c23)cc1 ; c1ccc(-c2ccc(N(c3ccc(-c4cccc5oc6ccccc6c45)cc3)c3ccc(-c4ccccc4)c4c3oc3ccccc34)cc2)cc1 ; c1ccc(-c2ccc(N(c3ccc(-c4cccc5oc6ccccc6c45)cc3)c3cccc4ccccc34)c3oc4ccccc4c23)cc1] 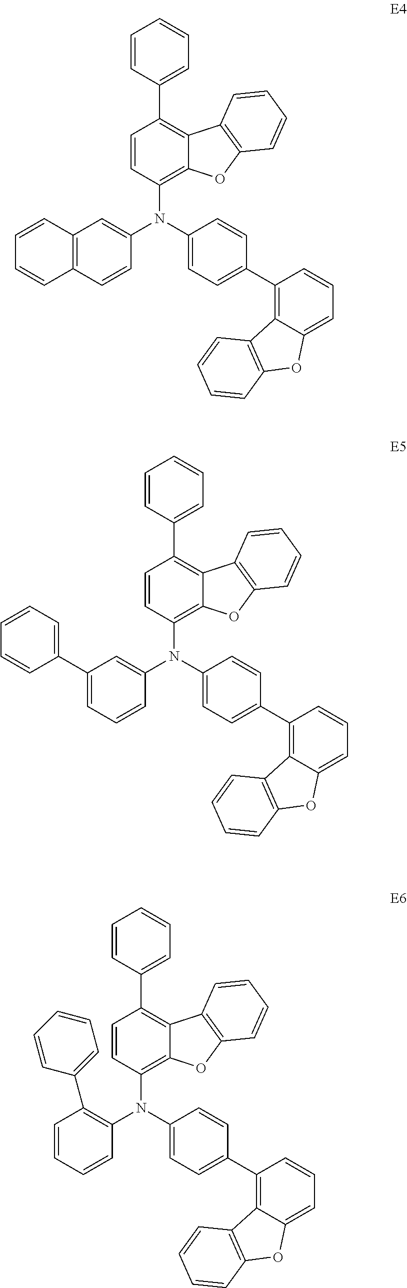 [SMILES: c1ccc(-c2ccc(N(c3ccc(-c4cccc5oc6ccccc6c45)cc3)c3ccc4ccccc4c3)c3oc4ccccc4c23)cc1 ; c1ccc(-c2cccc(N(c3ccc(-c4cccc5oc6ccccc6c45)cc3)c3ccc(-c4ccccc4)c4c3oc3ccccc34)c2)cc1 ; c1ccc(-c2ccccc2N(c2ccc(-c3cccc4oc5ccccc5c34)cc2)c2ccc(-c3ccccc3)c3c2oc2ccccc23)cc1] 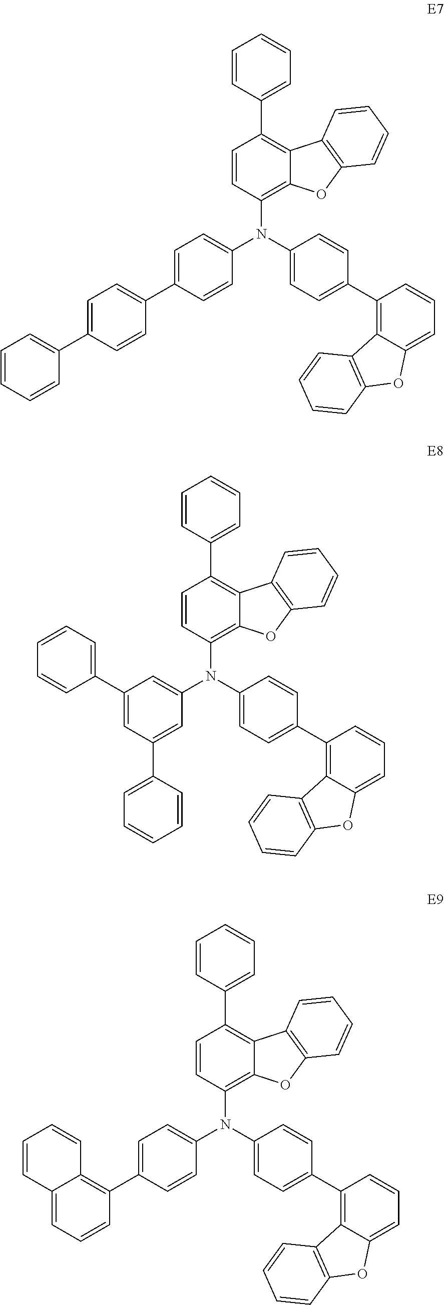 [SMILES: c1ccc(-c2ccc(-c3ccc(N(c4ccc(-c5cccc6oc7ccccc7c56)cc4)c4ccc(-c5ccccc5)c5c4oc4ccccc45)cc3)cc2)cc1 ; c1ccc(-c2cc(-c3ccccc3)cc(N(c3ccc(-c4cccc5oc6ccccc6c45)cc3)c3ccc(-c4ccccc4)c4c3oc3ccccc34)c2)cc1 ; c1ccc(-c2ccc(N(c3ccc(-c4cccc5ccccc45)cc3)c3ccc(-c4cccc5oc6ccccc6c45)cc3)c3oc4ccccc4c23)cc1] 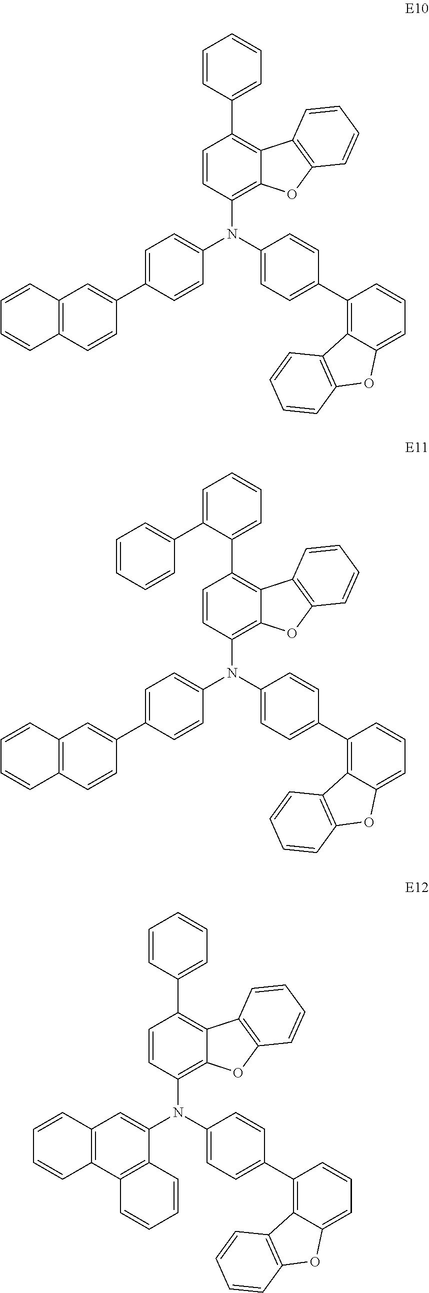 [SMILES: c1ccc(-c2ccc(N(c3ccc(-c4ccc5ccccc5c4)cc3)c3ccc(-c4cccc5oc6ccccc6c45)cc3)c3oc4ccccc4c23)cc1 ; c1ccc(-c2ccccc2-c2ccc(N(c3ccc(-c4ccc5ccccc5c4)cc3)c3ccc(-c4cccc5oc6ccccc6c45)cc3)c3oc4ccccc4c23)cc1 ; c1ccc(-c2ccc(N(c3ccc(-c4cccc5oc6ccccc6c45)cc3)c3cc4ccccc4c4ccccc34)c3oc4ccccc4c23)cc1] 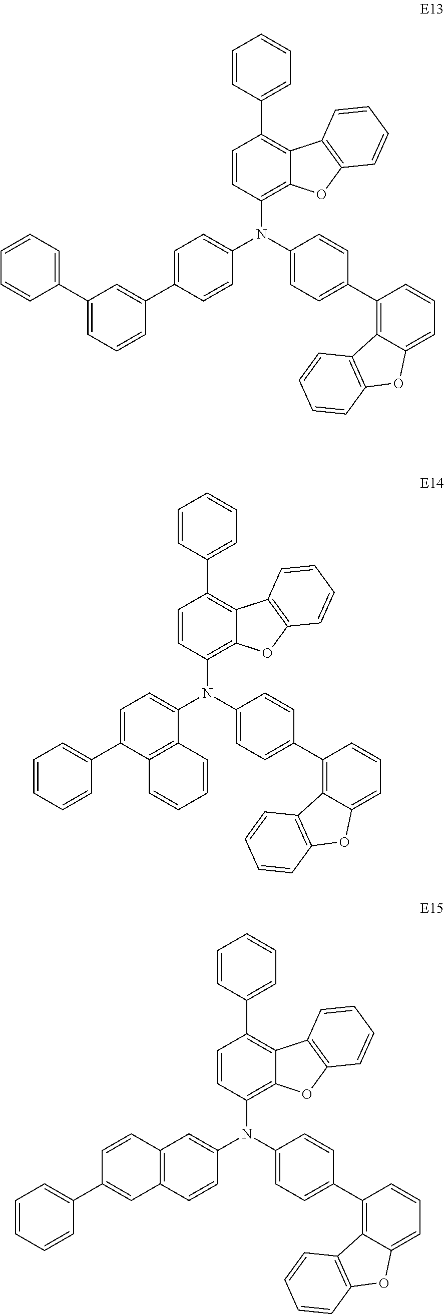 [SMILES: c1ccc(-c2cccc(-c3ccc(N(c4ccc(-c5cccc6oc7ccccc7c56)cc4)c4ccc(-c5ccccc5)c5c4oc4ccccc45)cc3)c2)cc1 ; c1ccc(-c2ccc(N(c3ccc(-c4cccc5oc6ccccc6c45)cc3)c3ccc(-c4ccccc4)c4c3oc3ccccc34)c3ccccc23)cc1 ; c1ccc(-c2ccc3cc(N(c4ccc(-c5cccc6oc7ccccc7c56)cc4)c4ccc(-c5ccccc5)c5c4oc4ccccc45)ccc3c2)cc1] 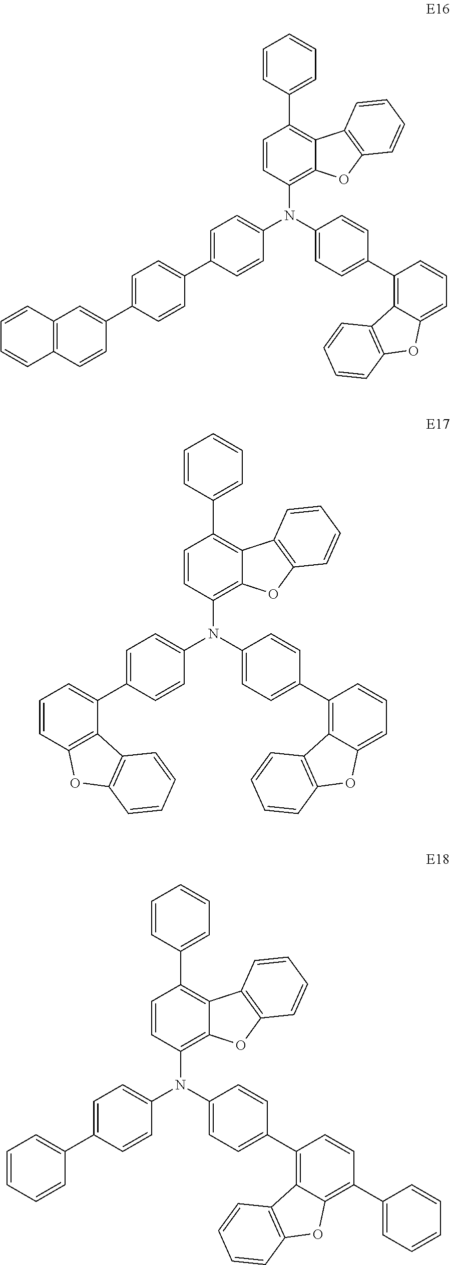 [SMILES: c1ccc(-c2ccc(N(c3ccc(-c4ccc(-c5ccc6ccccc6c5)cc4)cc3)c3ccc(-c4cccc5oc6ccccc6c45)cc3)c3oc4ccccc4c23)cc1 ; c1ccc(-c2ccc(N(c3ccc(-c4cccc5oc6ccccc6c45)cc3)c3ccc(-c4cccc5oc6ccccc6c45)cc3)c3oc4ccccc4c23)cc1 ; c1ccc(-c2ccc(N(c3ccc(-c4ccc(-c5ccccc5)c5oc6ccccc6c45)cc3)c3ccc(-c4ccccc4)c4c3oc3ccccc34)cc2)cc1] 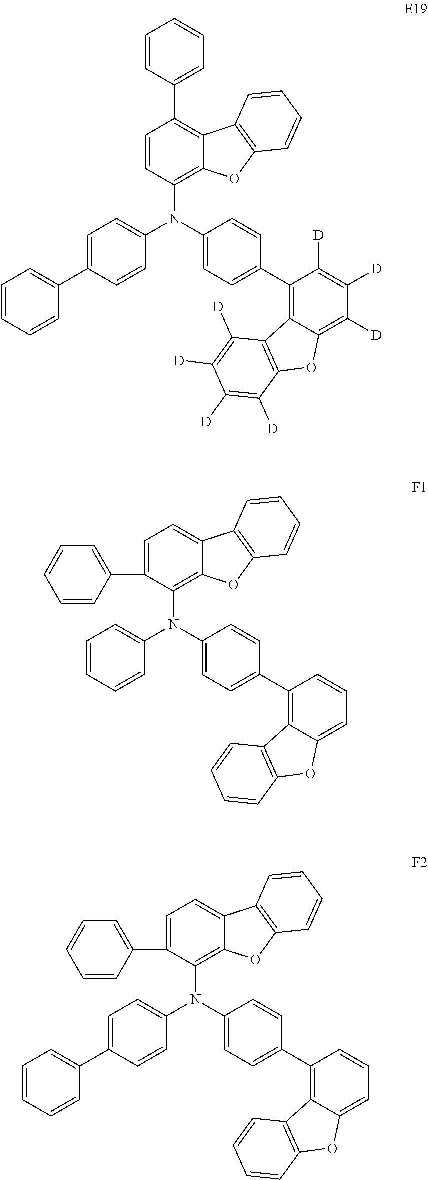 [SMILES: [2H]c1c([2H])c([2H])c2c(oc3c([2H])c([2H])c([2H])c(-c4ccc(N(c5ccc(-c6ccccc6)cc5)c5ccc(-c6ccccc6)c6c5oc5ccccc56)cc4)c32)c1[2H] ; c1ccc(-c2ccc3c(oc4ccccc43)c2N(c2ccccc2)c2ccc(-c3cccc4oc5ccccc5c34)cc2)cc1 ; c1ccc(-c2ccc(N(c3ccc(-c4cccc5oc6ccccc6c45)cc3)c3c(-c4ccccc4)ccc4c3oc3ccccc34)cc2)cc1] 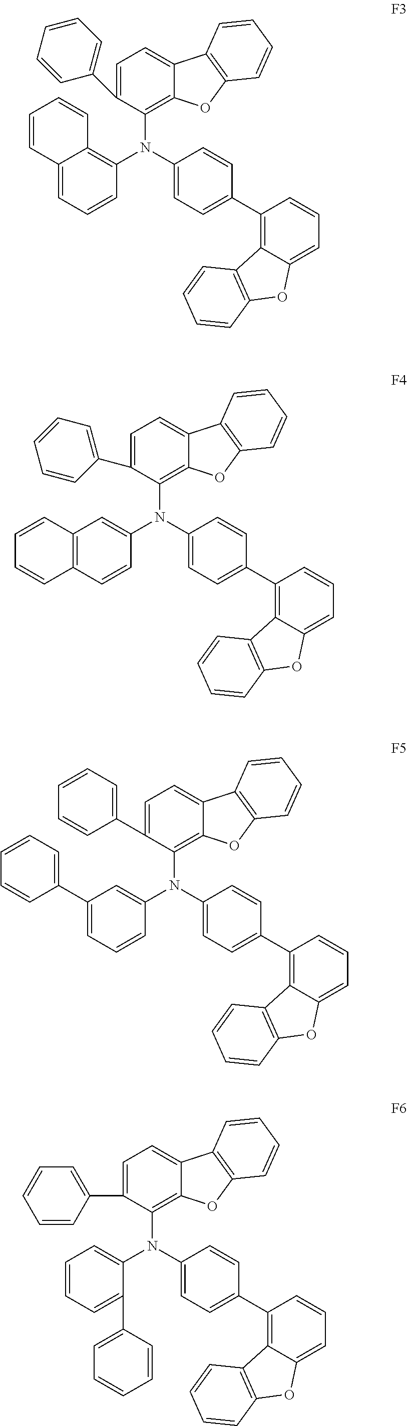 [SMILES: c1ccc(-c2ccc3c(oc4ccccc43)c2N(c2ccc(-c3cccc4oc5ccccc5c34)cc2)c2cccc3ccccc23)cc1 ; c1ccc(-c2ccc3c(oc4ccccc43)c2N(c2ccc(-c3cccc4oc5ccccc5c34)cc2)c2ccc3ccccc3c2)cc1 ; c1ccc(-c2cccc(N(c3ccc(-c4cccc5oc6ccccc6c45)cc3)c3c(-c4ccccc4)ccc4c3oc3ccccc34)c2)cc1 ; c1ccc(-c2ccccc2N(c2ccc(-c3cccc4oc5ccccc5c34)cc2)c2c(-c3ccccc3)ccc3c2oc2ccccc23)cc1] 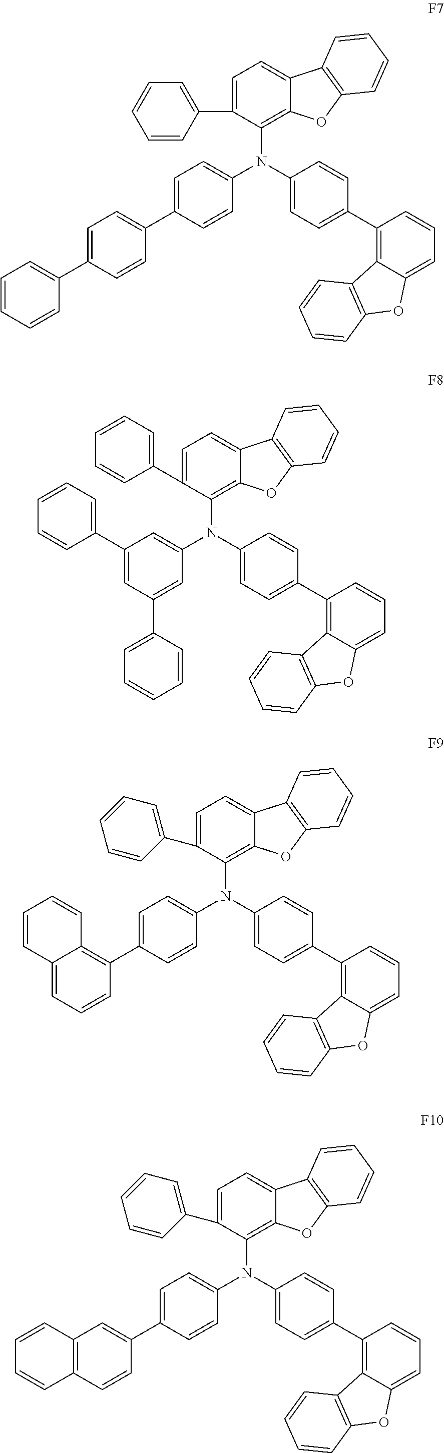 [SMILES: c1ccc(-c2ccc(-c3ccc(N(c4ccc(-c5cccc6oc7ccccc7c56)cc4)c4c(-c5ccccc5)ccc5c4oc4ccccc45)cc3)cc2)cc1 ; c1ccc(-c2cc(-c3ccccc3)cc(N(c3ccc(-c4cccc5oc6ccccc6c45)cc3)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc(-c3cccc4oc5ccccc5c34)cc2)cc1 ; c1ccc(-c2ccc3c(oc4ccccc43)c2N(c2ccc(-c3ccc4ccccc4c3)cc2)c2ccc(-c3cccc4oc5ccccc5c34)cc2)cc1] 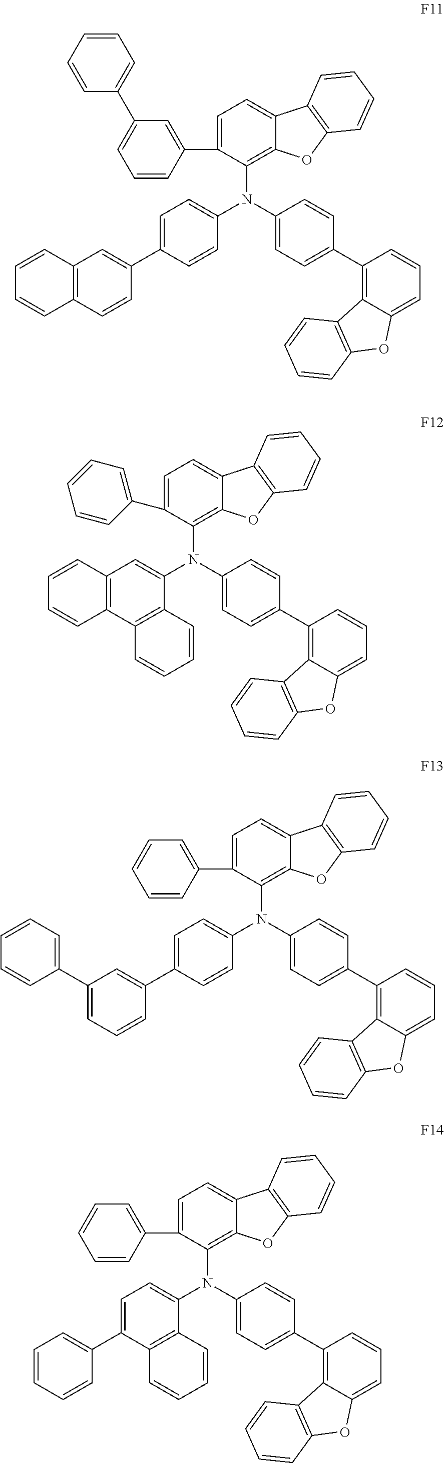 [SMILES: c1ccc(-c2cccc(-c3ccc4c(oc5ccccc54)c3N(c3ccc(-c4ccc5ccccc5c4)cc3)c3ccc(-c4cccc5oc6ccccc6c45)cc3)c2)cc1 ; c1ccc(-c2ccc3c(oc4ccccc43)c2N(c2ccc(-c3cccc4oc5ccccc5c34)cc2)c2cc3ccccc3c3ccccc23)cc1 ; c1ccc(-c2cccc(-c3ccc(N(c4ccc(-c5cccc6oc7ccccc7c56)cc4)c4c(-c5ccccc5)ccc5c4oc4ccccc45)cc3)c2)cc1 ; c1ccc(-c2ccc3c(oc4ccccc43)c2N(c2ccc(-c3cccc4oc5ccccc5c34)cc2)c2ccc(-c3ccccc3)c3ccccc23)cc1] 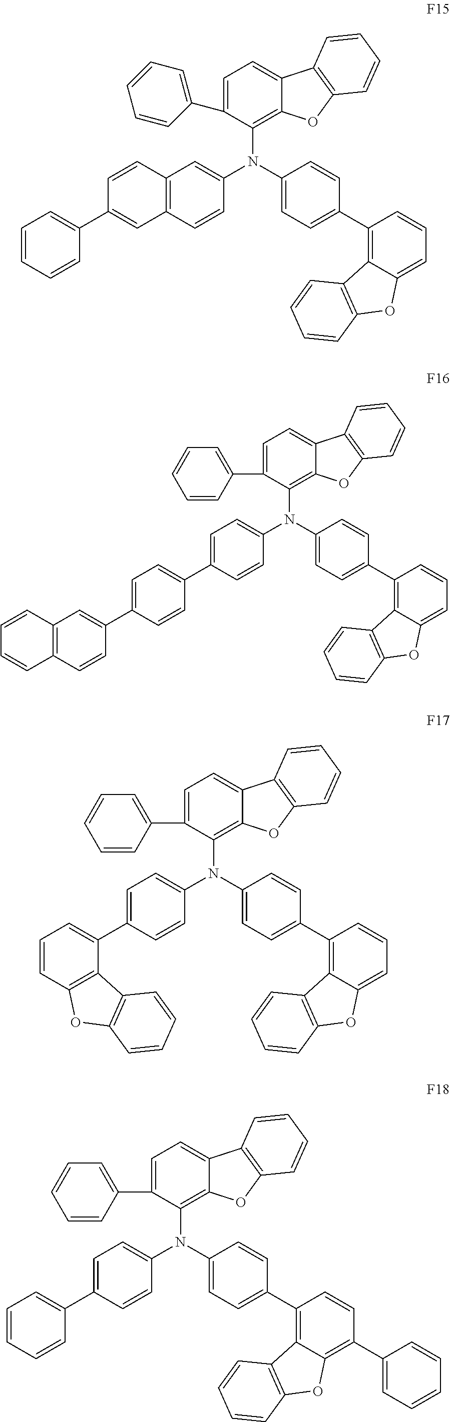 [SMILES: c1ccc(-c2ccc3cc(N(c4ccc(-c5cccc6oc7ccccc7c56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4oc5ccccc5c34)cc2)cc1 ; c1ccc(-c2ccc3c(oc4ccccc43)c2N(c2ccc(-c3cccc4oc5ccccc5c34)cc2)c2ccc(-c3cccc4oc5ccccc5c34)cc2)cc1 ; c1ccc(-c2ccc(N(c3ccc(-c4ccc(-c5ccccc5)c5oc6ccccc6c45)cc3)c3c(-c4ccccc4)ccc4c3oc3ccccc34)cc2)cc1] 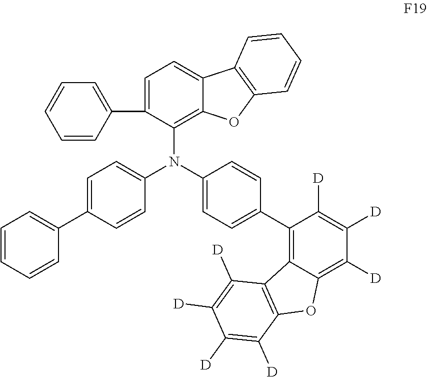 [SMILES: [2H]c1c([2H])c([2H])c2c(oc3c([2H])c([2H])c([2H])c(-c4ccc(N(c5ccc(-c6ccccc6)cc5)c5c(-c6ccccc6)ccc6c5oc5ccccc56)cc4)c32)c1[2H]] 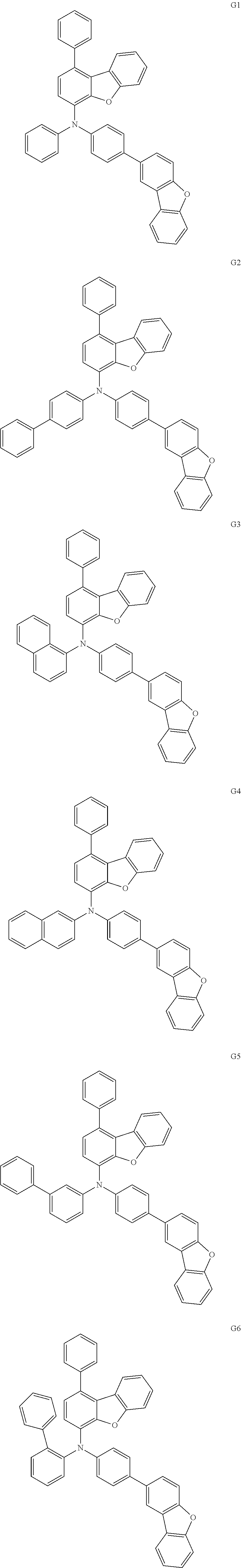 [SMILES: c1ccc(-c2ccc(N(c3ccccc3)c3ccc(-c4ccc5oc6ccccc6c5c4)cc3)c3oc4ccccc4c23)cc1 ; c1ccc(-c2ccc(N(c3ccc(-c4ccc5oc6ccccc6c5c4)cc3)c3ccc(-c4ccccc4)c4c3oc3ccccc34)cc2)cc1 ; c1ccc(-c2ccc(N(c3ccc(-c4ccc5oc6ccccc6c5c4)cc3)c3cccc4ccccc34)c3oc4ccccc4c23)cc1 ; c1ccc(-c2ccc(N(c3ccc(-c4ccc5oc6ccccc6c5c4)cc3)c3ccc4ccccc4c3)c3oc4ccccc4c23)cc1 ; c1ccc(-c2cccc(N(c3ccc(-c4ccc5oc6ccccc6c5c4)cc3)c3ccc(-c4ccccc4)c4c3oc3ccccc34)c2)cc1 ; c1ccc(-c2ccccc2N(c2ccc(-c3ccc4oc5ccccc5c4c3)cc2)c2ccc(-c3ccccc3)c3c2oc2ccccc23)cc1] 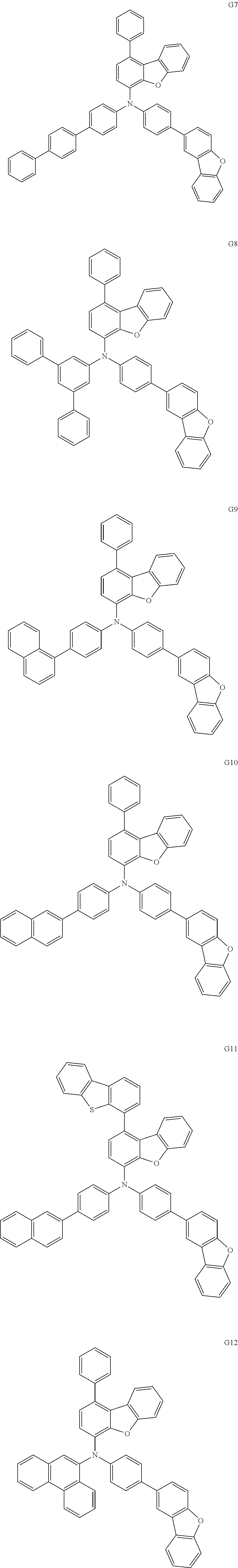 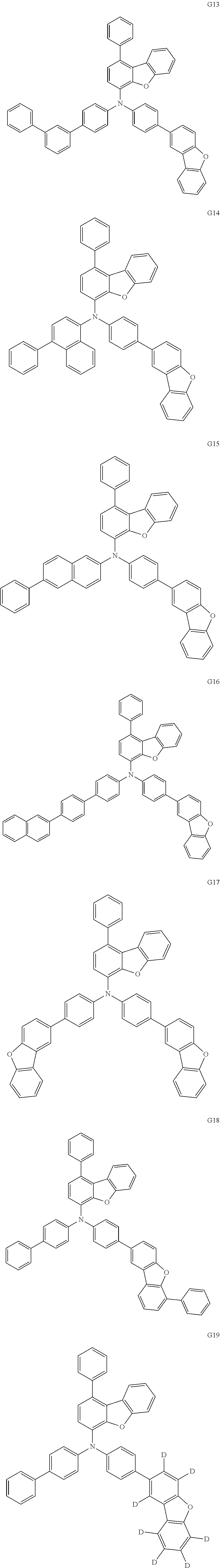 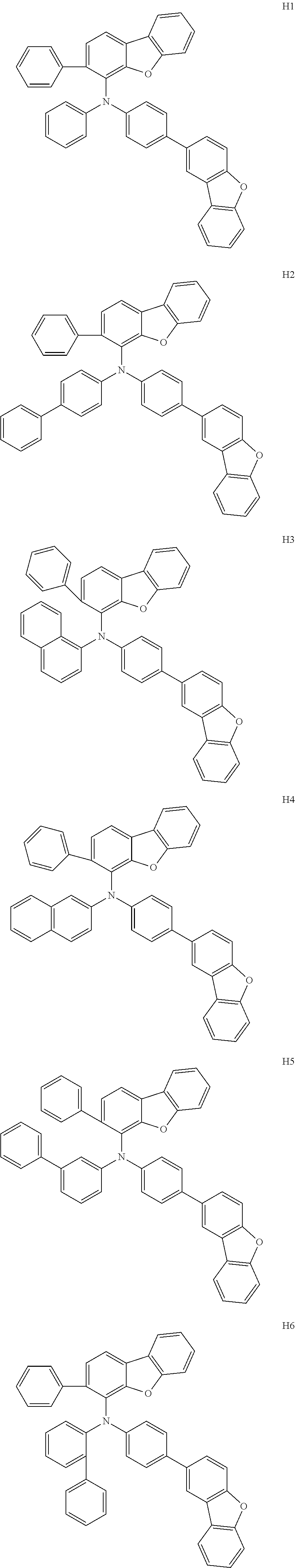 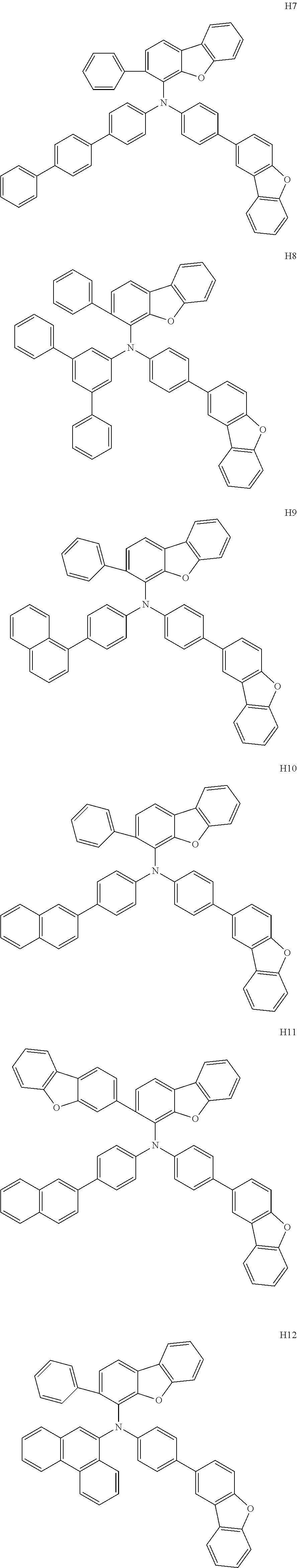 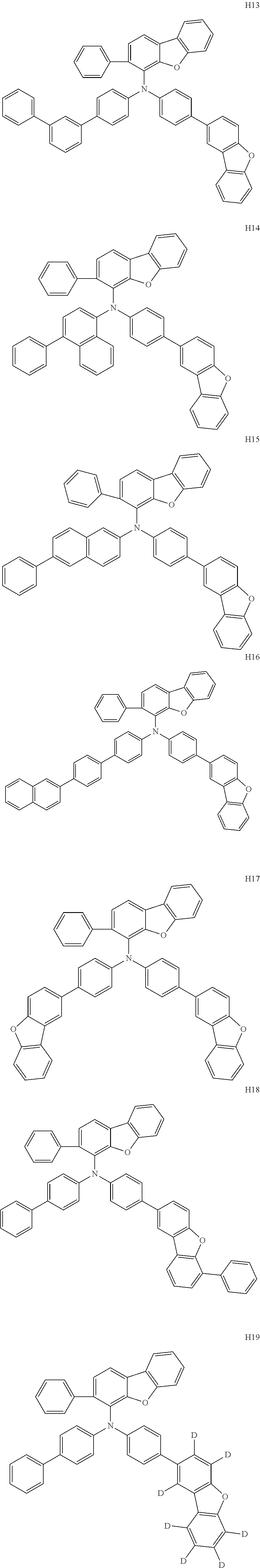 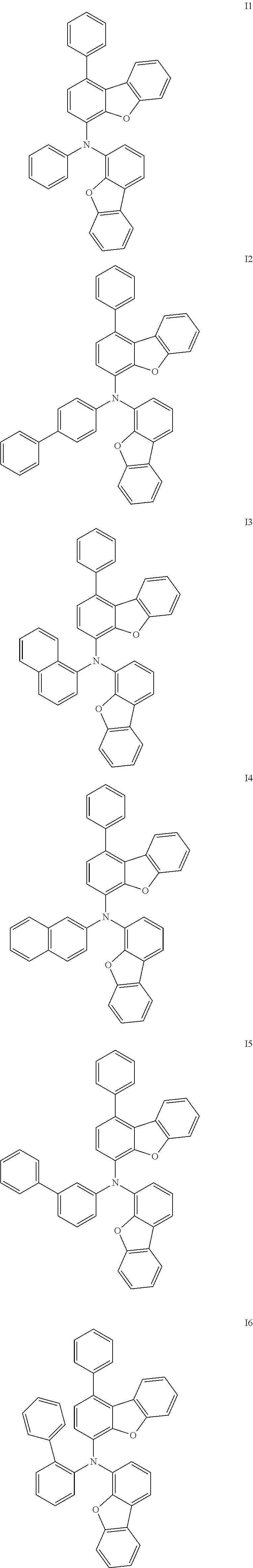 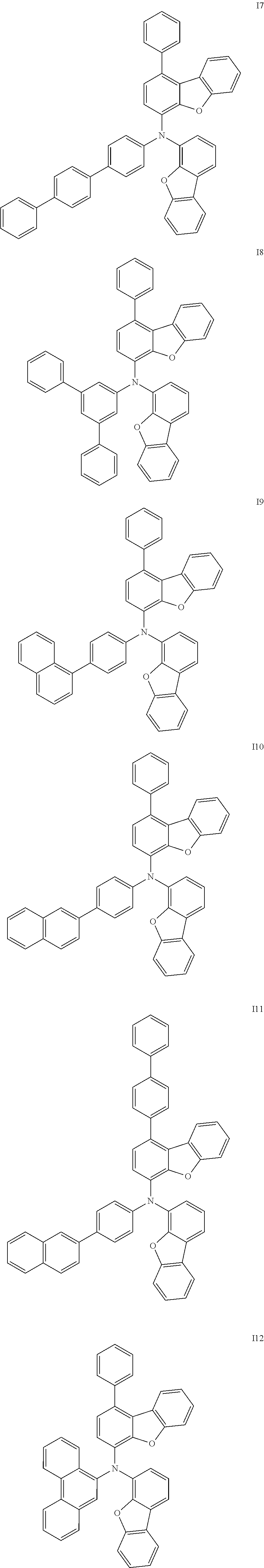 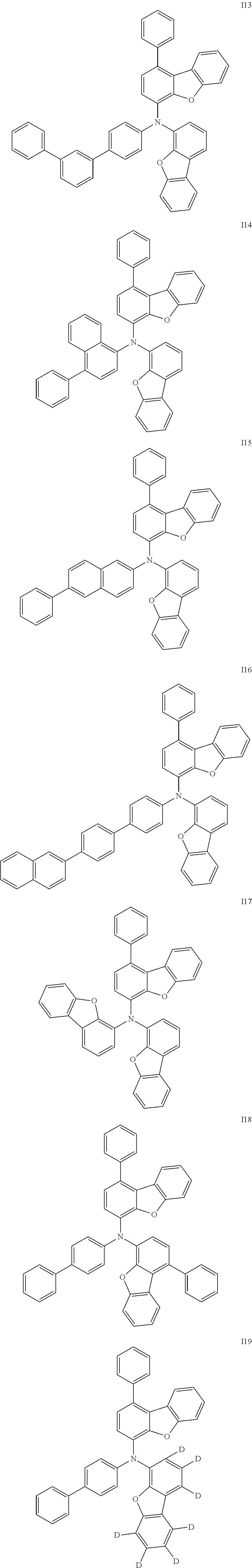 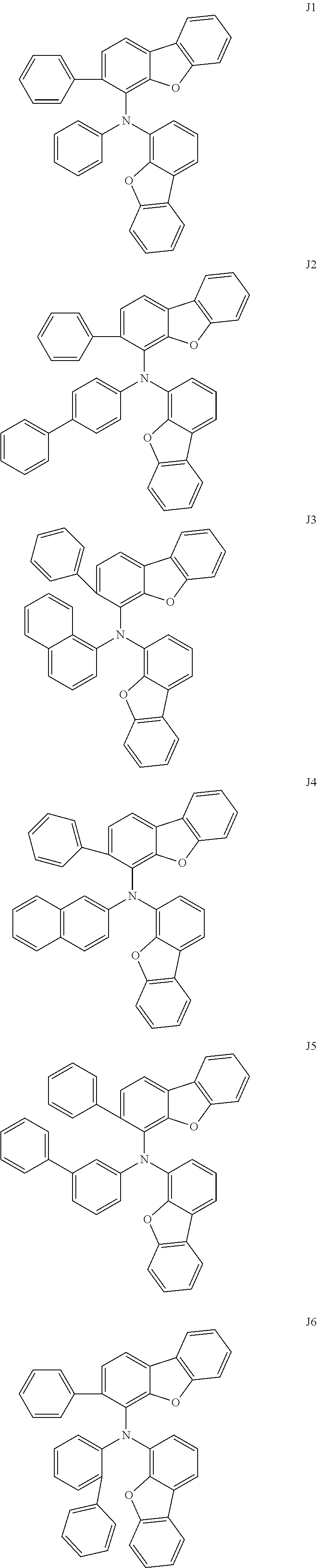 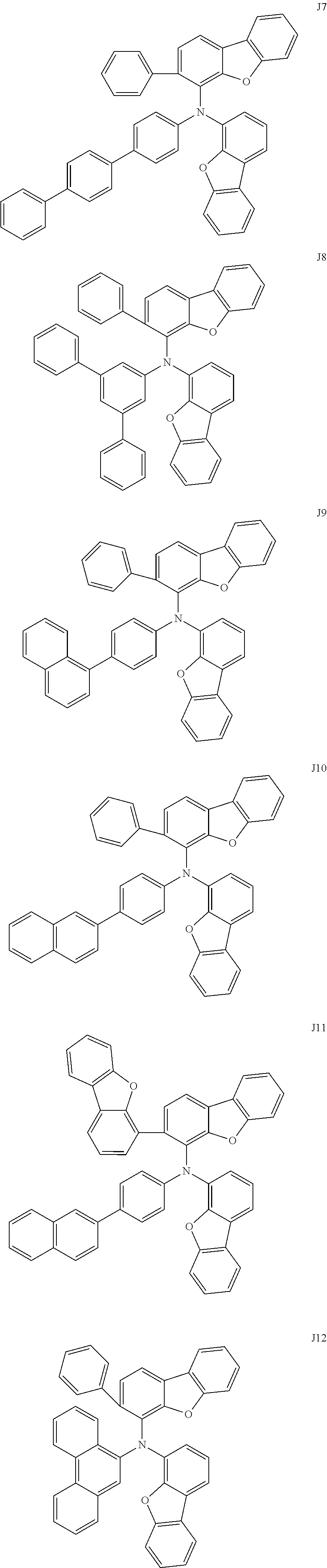 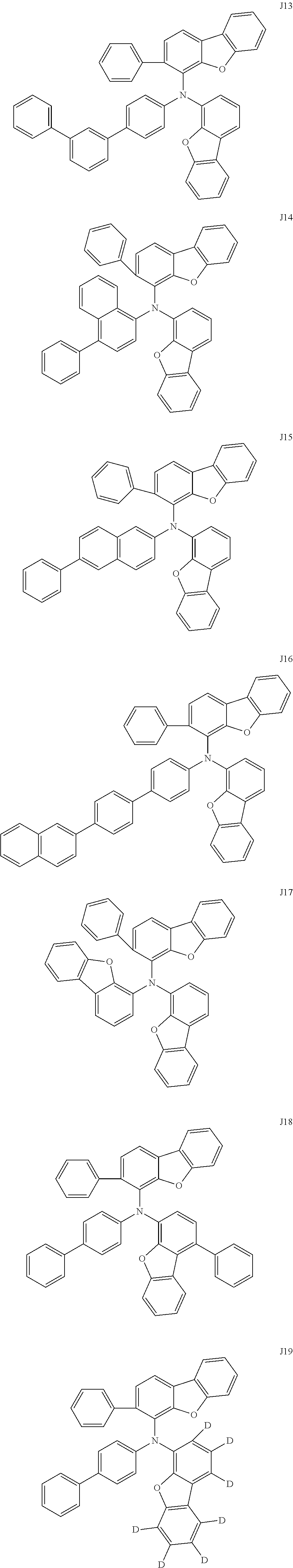 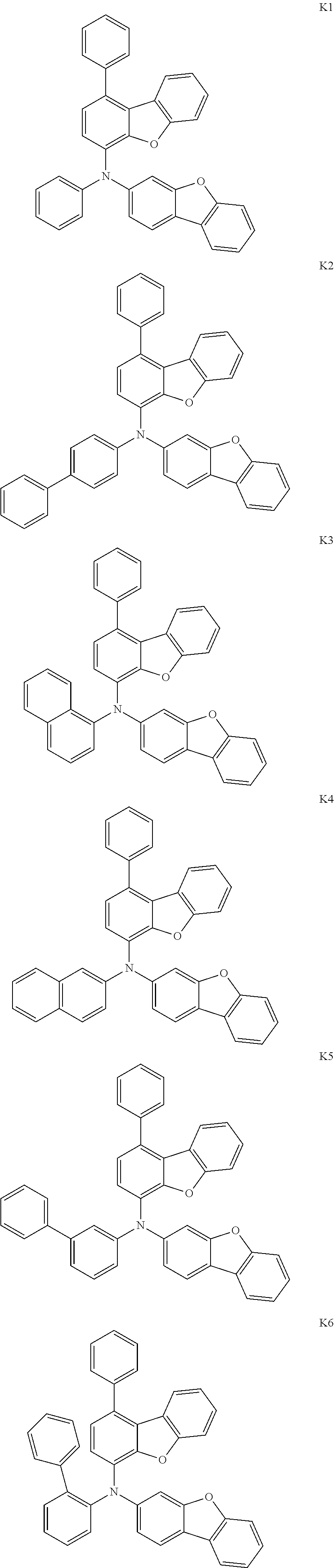 [SMILES: c1ccc(-c2ccc(N(c3ccccc3)c3ccc4c(c3)oc3ccccc34)c3oc4ccccc4c23)cc1 ; c1ccc(-c2ccc(N(c3ccc4c(c3)oc3ccccc34)c3ccc(-c4ccccc4)c4c3oc3ccccc34)cc2)cc1 ; c1ccc(-c2ccc(N(c3ccc4c(c3)oc3ccccc34)c3cccc4ccccc34)c3oc4ccccc4c23)cc1 ; c1ccc(-c2ccc(N(c3ccc4ccccc4c3)c3ccc4c(c3)oc3ccccc34)c3oc4ccccc4c23)cc1 ; c1ccc(-c2cccc(N(c3ccc4c(c3)oc3ccccc34)c3ccc(-c4ccccc4)c4c3oc3ccccc34)c2)cc1 ; c1ccc(-c2ccccc2N(c2ccc3c(c2)oc2ccccc23)c2ccc(-c3ccccc3)c3c2oc2ccccc23)cc1] 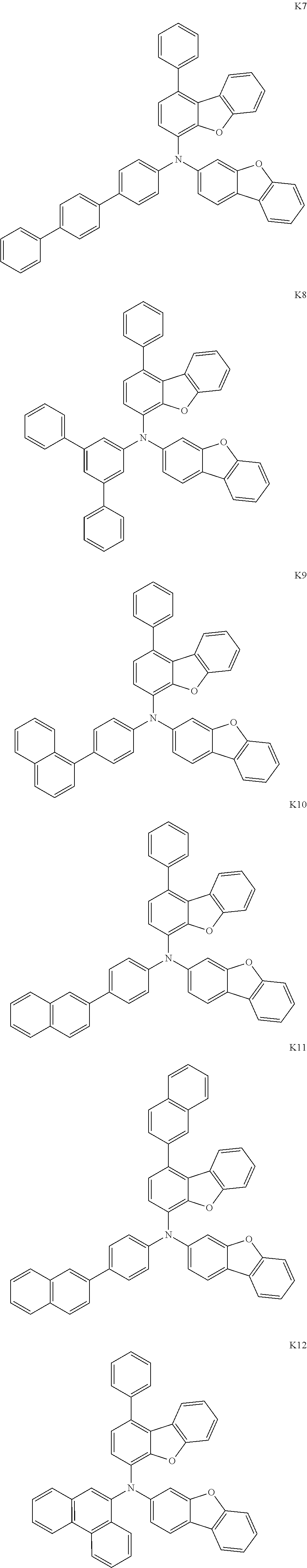 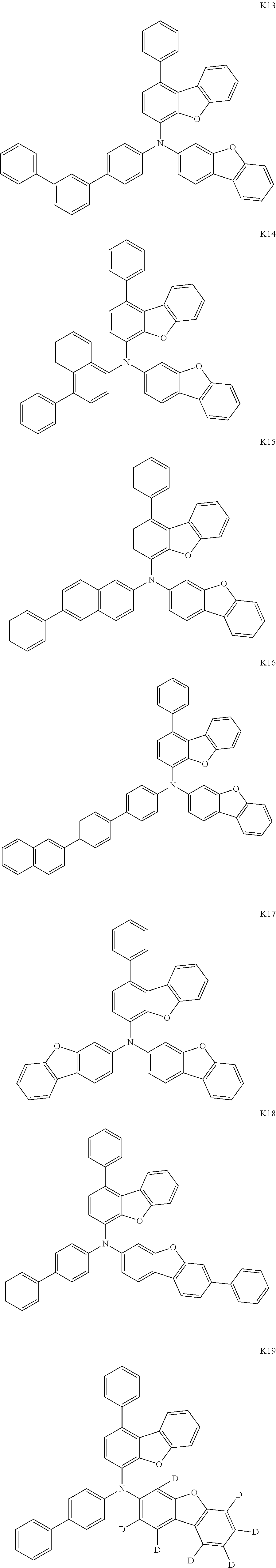 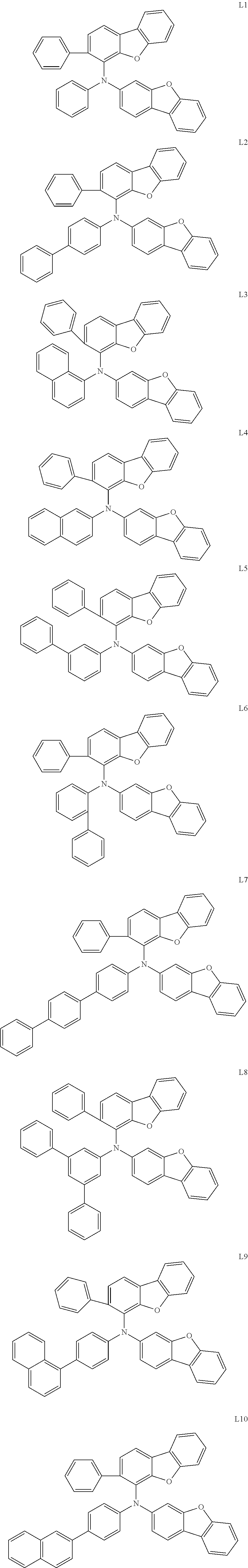 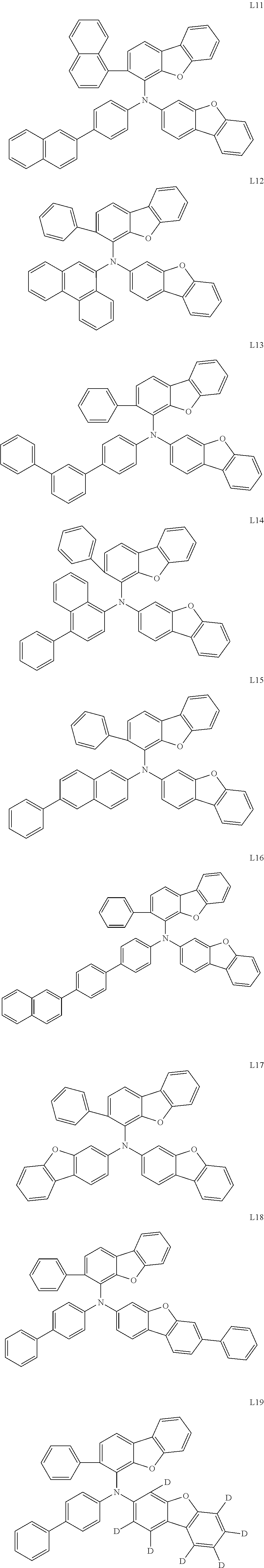 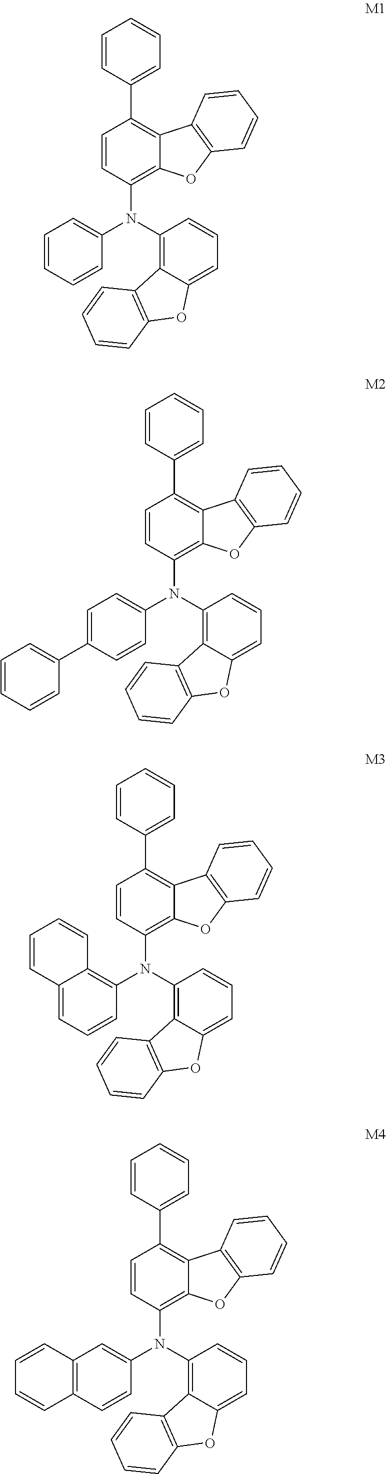 [SMILES: c1ccc(-c2ccc(N(c3ccccc3)c3cccc4oc5ccccc5c34)c3oc4ccccc4c23)cc1 ; c1ccc(-c2ccc(N(c3ccc(-c4ccccc4)c4c3oc3ccccc34)c3cccc4oc5ccccc5c34)cc2)cc1 ; c1ccc(-c2ccc(N(c3cccc4ccccc34)c3cccc4oc5ccccc5c34)c3oc4ccccc4c23)cc1 ; c1ccc(-c2ccc(N(c3ccc4ccccc4c3)c3cccc4oc5ccccc5c34)c3oc4ccccc4c23)cc1] 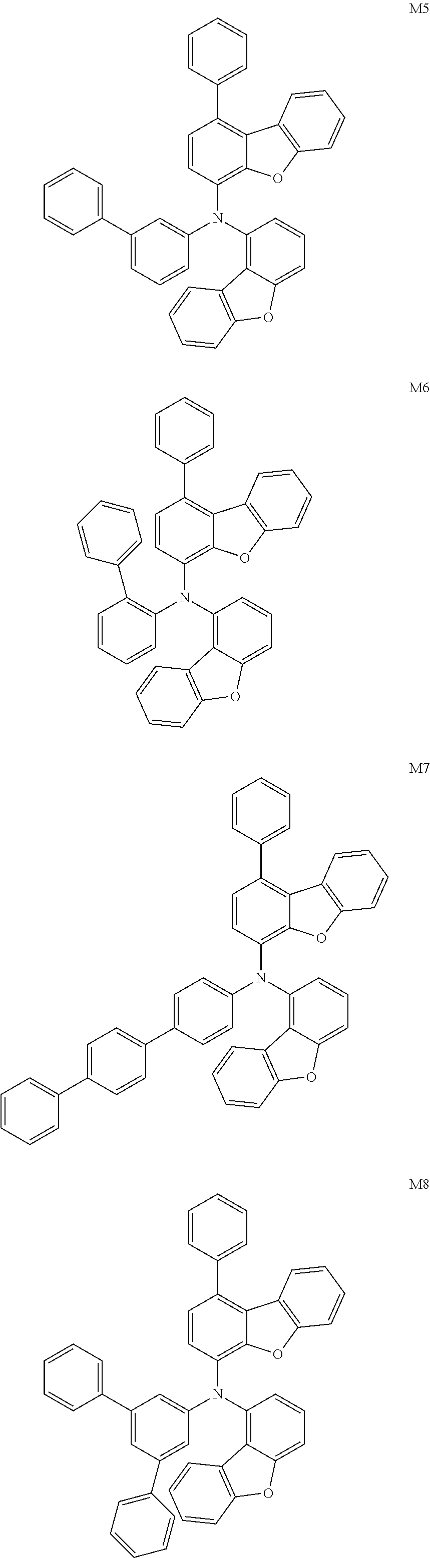 [SMILES: c1ccc(-c2cccc(N(c3ccc(-c4ccccc4)c4c3oc3ccccc34)c3cccc4oc5ccccc5c34)c2)cc1 ; c1ccc(-c2ccccc2N(c2ccc(-c3ccccc3)c3c2oc2ccccc23)c2cccc3oc4ccccc4c23)cc1 ; c1ccc(-c2ccc(-c3ccc(N(c4ccc(-c5ccccc5)c5c4oc4ccccc45)c4cccc5oc6ccccc6c45)cc3)cc2)cc1 ; c1ccc(-c2cc(-c3ccccc3)cc(N(c3ccc(-c4ccccc4)c4c3oc3ccccc34)c3cccc4oc5ccccc5c34)c2)cc1] 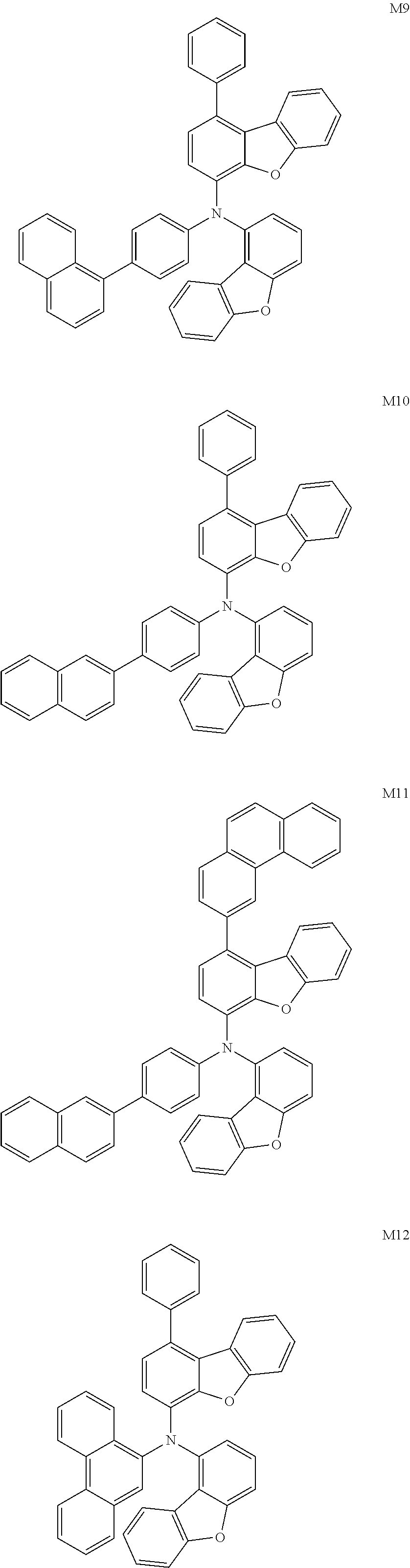 [SMILES: c1ccc(-c2ccc(N(c3ccc(-c4cccc5ccccc45)cc3)c3cccc4oc5ccccc5c34)c3oc4ccccc4c23)cc1 ; c1ccc(-c2ccc(N(c3ccc(-c4ccc5ccccc5c4)cc3)c3cccc4oc5ccccc5c34)c3oc4ccccc4c23)cc1 ; c1ccc2cc(-c3ccc(N(c4ccc(-c5ccc6ccc7ccccc7c6c5)c5c4oc4ccccc45)c4cccc5oc6ccccc6c45)cc3)ccc2c1 ; c1ccc(-c2ccc(N(c3cc4ccccc4c4ccccc34)c3cccc4oc5ccccc5c34)c3oc4ccccc4c23)cc1] 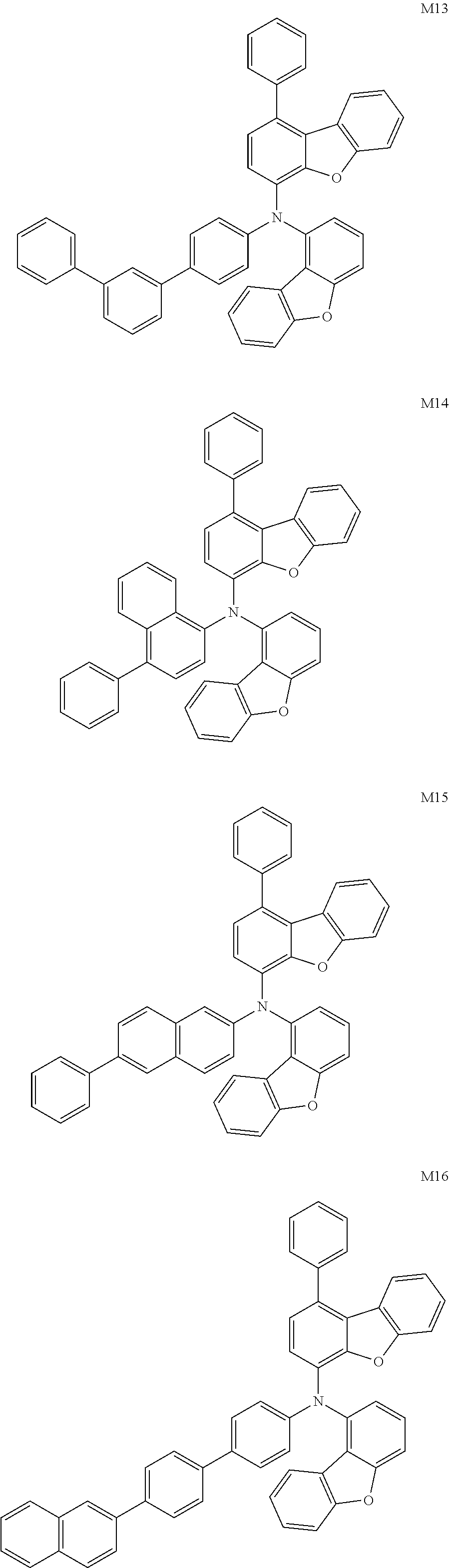 [SMILES: c1ccc(-c2cccc(-c3ccc(N(c4ccc(-c5ccccc5)c5c4oc4ccccc45)c4cccc5oc6ccccc6c45)cc3)c2)cc1 ; c1ccc(-c2ccc(N(c3ccc(-c4ccccc4)c4c3oc3ccccc34)c3cccc4oc5ccccc5c34)c3ccccc23)cc1 ; c1ccc(-c2ccc3cc(N(c4ccc(-c5ccccc5)c5c4oc4ccccc45)c4cccc5oc6ccccc6c45)ccc3c2)cc1 ; c1ccc(-c2ccc(N(c3ccc(-c4ccc(-c5ccc6ccccc6c5)cc4)cc3)c3cccc4oc5ccccc5c34)c3oc4ccccc4c23)cc1] 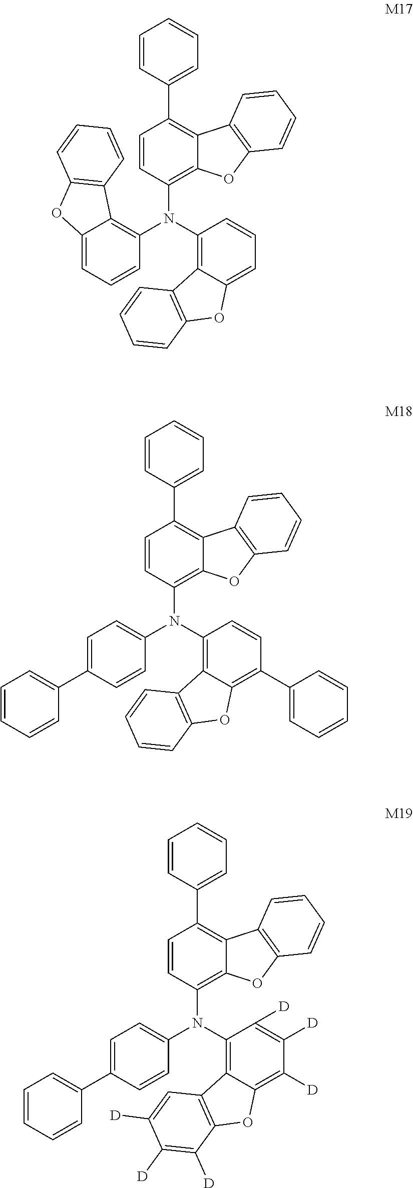 [SMILES: c1ccc(-c2ccc(N(c3cccc4oc5ccccc5c34)c3cccc4oc5ccccc5c34)c3oc4ccccc4c23)cc1 ; c1ccc(-c2ccc(N(c3ccc(-c4ccccc4)c4c3oc3ccccc34)c3ccc(-c4ccccc4)c4oc5ccccc5c34)cc2)cc1 ; [2H]c1cc2c(oc3c([2H])c([2H])c([2H])c(N(c4ccc(-c5ccccc5)cc4)c4ccc(-c5ccccc5)c5c4oc4ccccc45)c32)c([2H])c1[2H]] 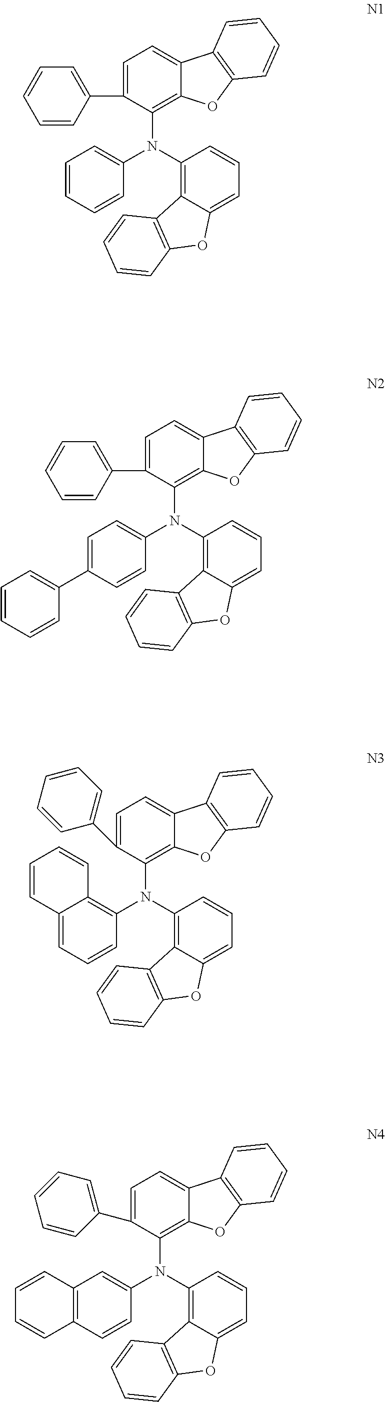 [SMILES: c1ccc(-c2ccc3c(oc4ccccc43)c2N(c2ccccc2)c2cccc3oc4ccccc4c23)cc1 ; c1ccc(-c2ccc(N(c3c(-c4ccccc4)ccc4c3oc3ccccc34)c3cccc4oc5ccccc5c34)cc2)cc1 ; c1ccc(-c2ccc3c(oc4ccccc43)c2N(c2cccc3ccccc23)c2cccc3oc4ccccc4c23)cc1 ; c1ccc(-c2ccc3c(oc4ccccc43)c2N(c2ccc3ccccc3c2)c2cccc3oc4ccccc4c23)cc1] 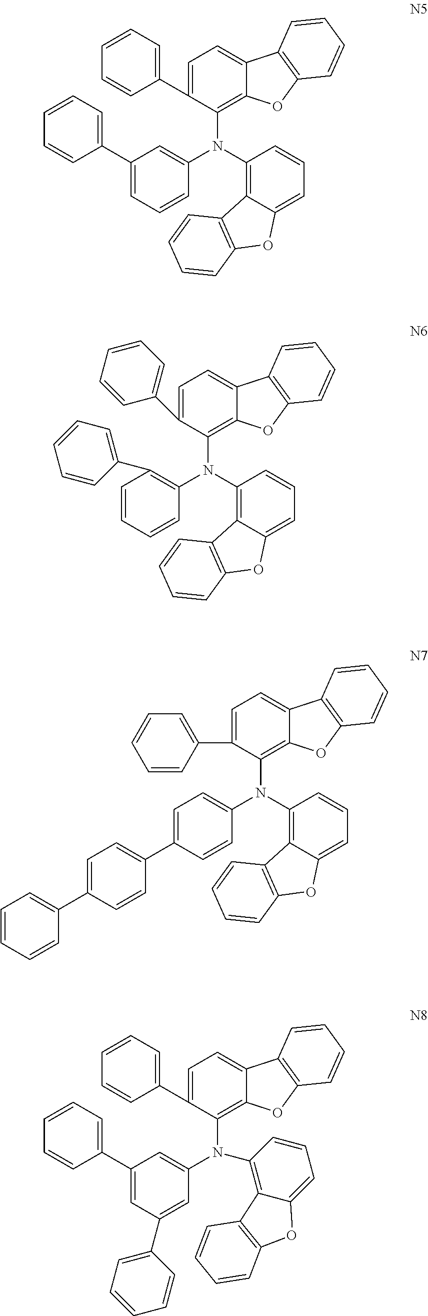 [SMILES: c1ccc(-c2cccc(N(c3c(-c4ccccc4)ccc4c3oc3ccccc34)c3cccc4oc5ccccc5c34)c2)cc1 ; c1ccc(-c2ccccc2N(c2c(-c3ccccc3)ccc3c2oc2ccccc23)c2cccc3oc4ccccc4c23)cc1 ; c1ccc(-c2ccc(-c3ccc(N(c4c(-c5ccccc5)ccc5c4oc4ccccc45)c4cccc5oc6ccccc6c45)cc3)cc2)cc1 ; c1ccc(-c2cc(-c3ccccc3)cc(N(c3c(-c4ccccc4)ccc4c3oc3ccccc34)c3cccc4oc5ccccc5c34)c2)cc1] 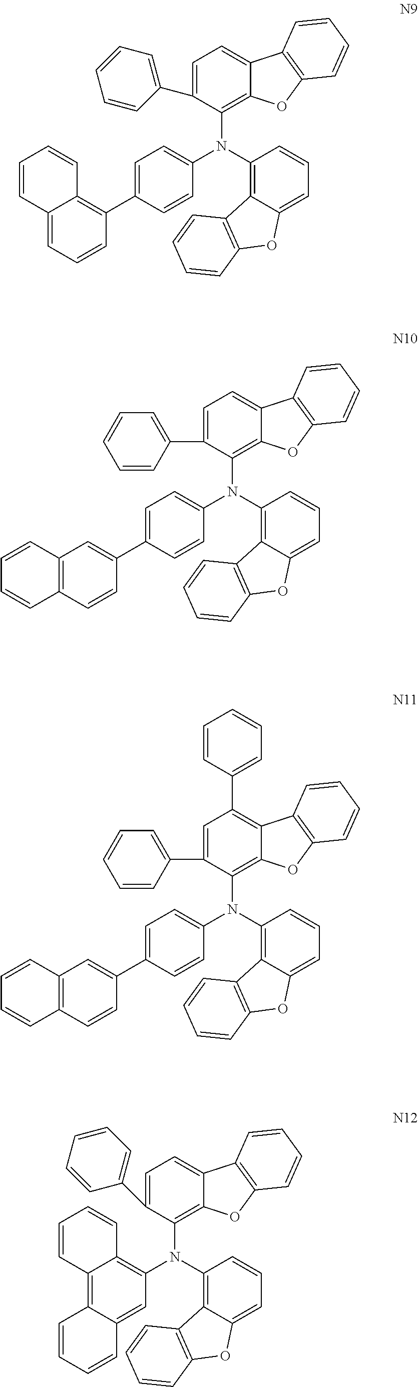 [SMILES: c1ccc(-c2ccc3c(oc4ccccc43)c2N(c2ccc(-c3cccc4ccccc34)cc2)c2cccc3oc4ccccc4c23)cc1 ; c1ccc(-c2ccc3c(oc4ccccc43)c2N(c2ccc(-c3ccc4ccccc4c3)cc2)c2cccc3oc4ccccc4c23)cc1 ; c1ccc(-c2cc(-c3ccccc3)c3c(oc4ccccc43)c2N(c2ccc(-c3ccc4ccccc4c3)cc2)c2cccc3oc4ccccc4c23)cc1 ; c1ccc(-c2ccc3c(oc4ccccc43)c2N(c2cc3ccccc3c3ccccc23)c2cccc3oc4ccccc4c23)cc1] 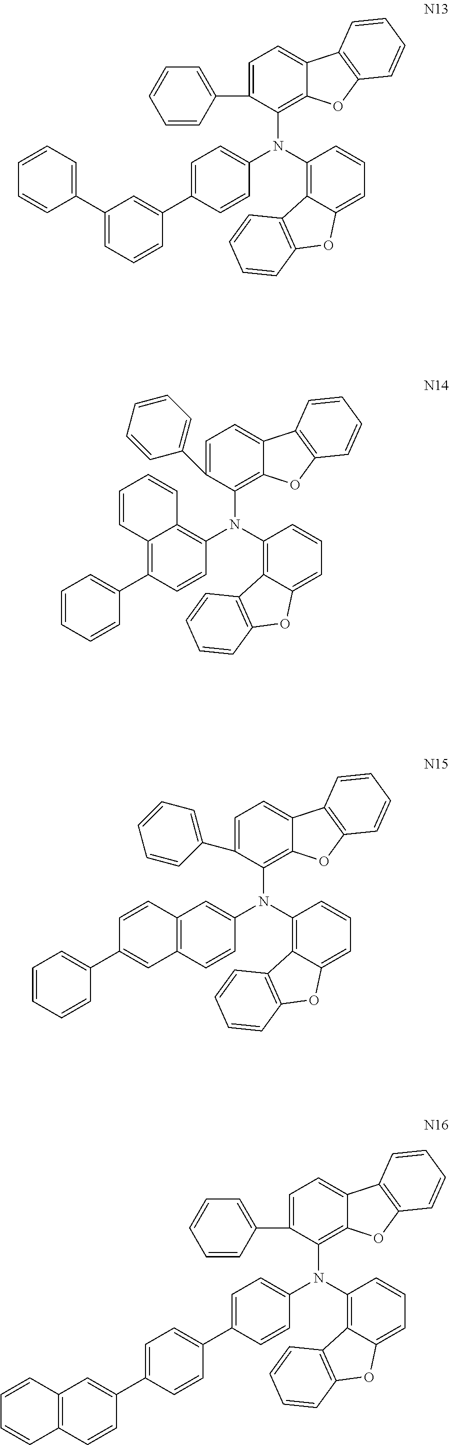 [SMILES: c1ccc(-c2cccc(-c3ccc(N(c4c(-c5ccccc5)ccc5c4oc4ccccc45)c4cccc5oc6ccccc6c45)cc3)c2)cc1 ; c1ccc(-c2ccc3c(oc4ccccc43)c2N(c2ccc(-c3ccccc3)c3ccccc23)c2cccc3oc4ccccc4c23)cc1 ; c1ccc(-c2ccc3cc(N(c4c(-c5ccccc5)ccc5c4oc4ccccc45)c4cccc5oc6ccccc6c45)ccc3c2)cc1 ; c1ccc(-c2ccc3c(oc4ccccc43)c2N(c2ccc(-c3ccc(-c4ccc5ccccc5c4)cc3)cc2)c2cccc3oc4ccccc4c23)cc1] 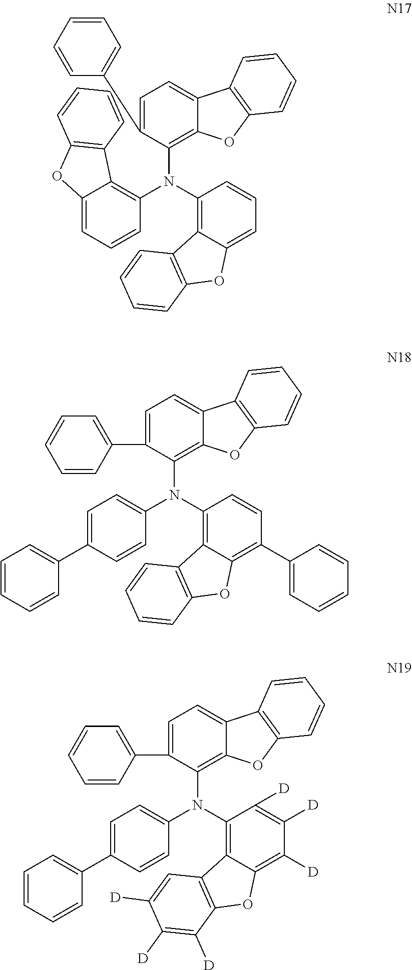 [SMILES: c1ccc(-c2ccc3c(oc4ccccc43)c2N(c2cccc3oc4ccccc4c23)c2cccc3oc4ccccc4c23)cc1 ; c1ccc(-c2ccc(N(c3c(-c4ccccc4)ccc4c3oc3ccccc34)c3ccc(-c4ccccc4)c4oc5ccccc5c34)cc2)cc1 ; [2H]c1cc2c(oc3c([2H])c([2H])c([2H])c(N(c4ccc(-c5ccccc5)cc4)c4c(-c5ccccc5)ccc5c4oc4ccccc45)c32)c([2H])c1[2H]] 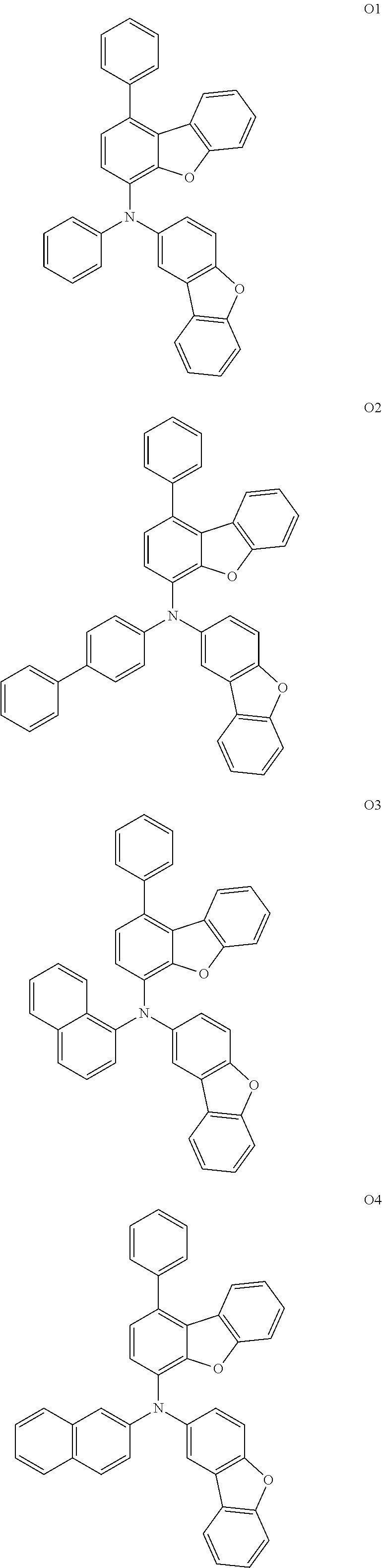 [SMILES: c1ccc(-c2ccc(N(c3ccccc3)c3ccc4oc5ccccc5c4c3)c3oc4ccccc4c23)cc1 ; c1ccc(-c2ccc(N(c3ccc4oc5ccccc5c4c3)c3ccc(-c4ccccc4)c4c3oc3ccccc34)cc2)cc1 ; c1ccc(-c2ccc(N(c3ccc4oc5ccccc5c4c3)c3cccc4ccccc34)c3oc4ccccc4c23)cc1 ; c1ccc(-c2ccc(N(c3ccc4ccccc4c3)c3ccc4oc5ccccc5c4c3)c3oc4ccccc4c23)cc1] 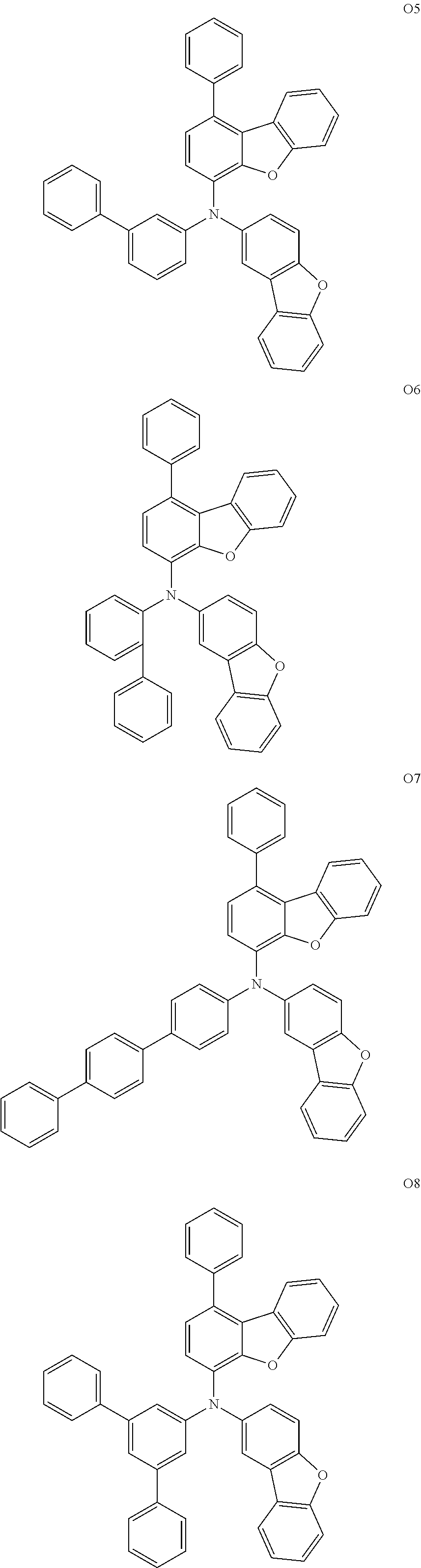 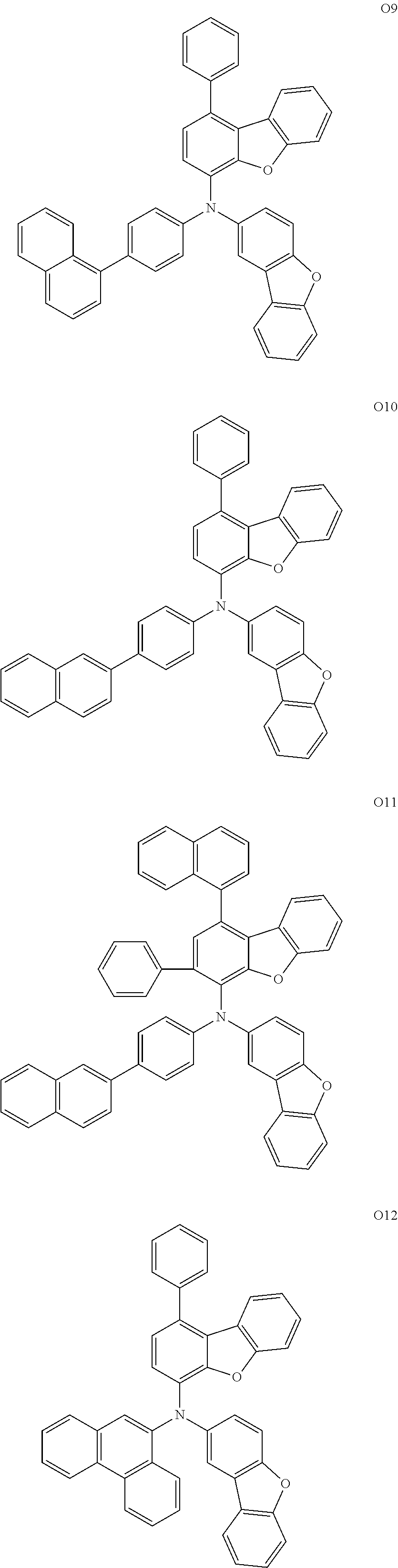 [SMILES: c1ccc(-c2ccc(N(c3ccc(-c4cccc5ccccc45)cc3)c3ccc4oc5ccccc5c4c3)c3oc4ccccc4c23)cc1 ; c1ccc(-c2ccc(N(c3ccc(-c4ccc5ccccc5c4)cc3)c3ccc4oc5ccccc5c4c3)c3oc4ccccc4c23)cc1 ; c1ccc(-c2cc(-c3cccc4ccccc34)c3c(oc4ccccc43)c2N(c2ccc(-c3ccc4ccccc4c3)cc2)c2ccc3oc4ccccc4c3c2)cc1 ; c1ccc(-c2ccc(N(c3ccc4oc5ccccc5c4c3)c3cc4ccccc4c4ccccc34)c3oc4ccccc4c23)cc1] 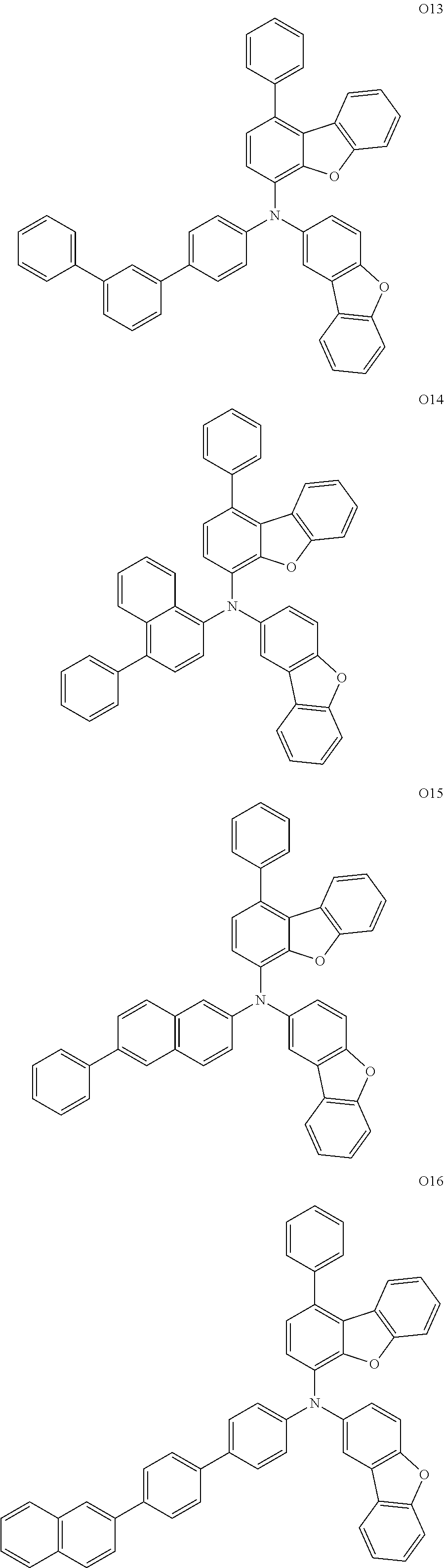 [SMILES: c1ccc(-c2cccc(-c3ccc(N(c4ccc5oc6ccccc6c5c4)c4ccc(-c5ccccc5)c5c4oc4ccccc45)cc3)c2)cc1 ; c1ccc(-c2ccc(N(c3ccc4oc5ccccc5c4c3)c3ccc(-c4ccccc4)c4c3oc3ccccc34)c3ccccc23)cc1 ; c1ccc(-c2ccc3cc(N(c4ccc5oc6ccccc6c5c4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc4oc5ccccc5c4c3)c3oc4ccccc4c23)cc1] 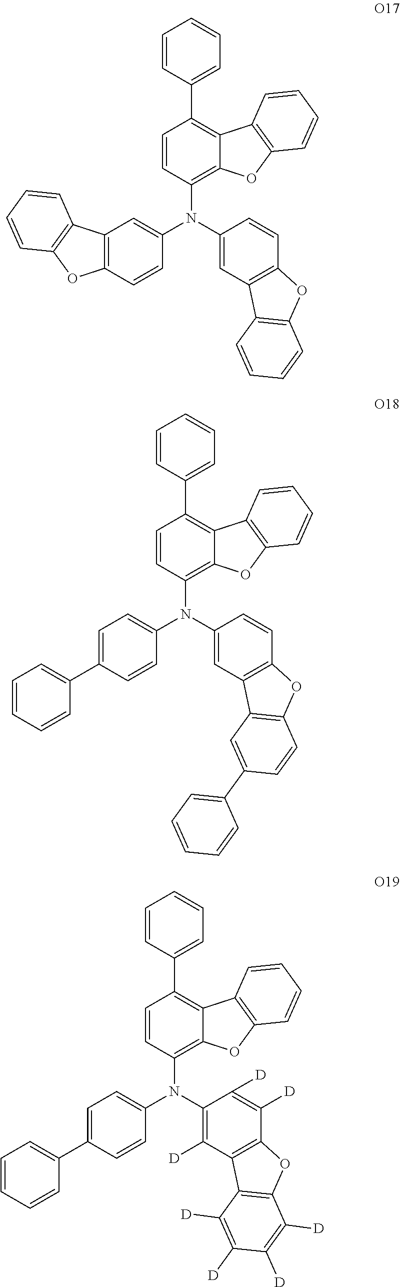 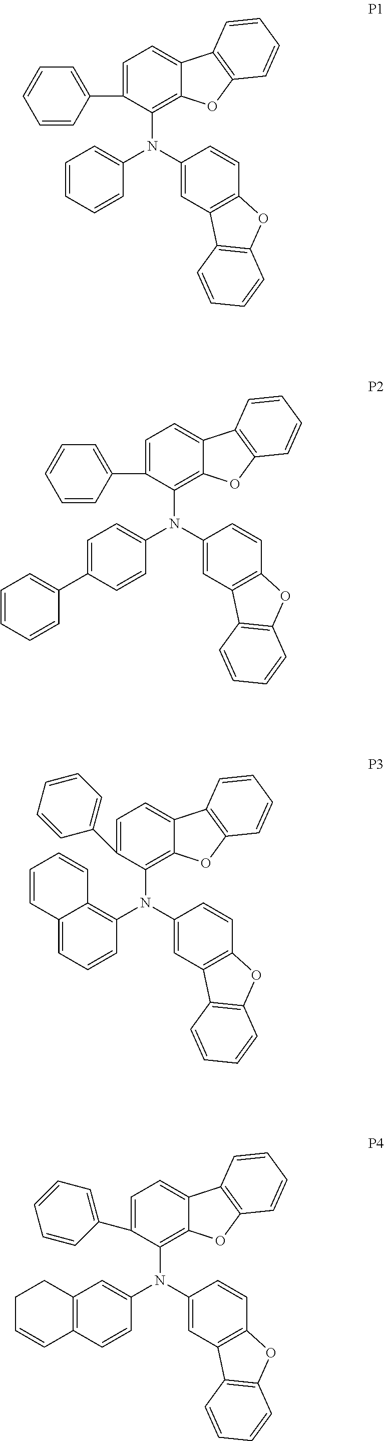 [SMILES: c1ccc(-c2ccc3c(oc4ccccc43)c2N(c2ccccc2)c2ccc3oc4ccccc4c3c2)cc1 ; c1ccc(-c2ccc(N(c3ccc4oc5ccccc5c4c3)c3c(-c4ccccc4)ccc4c3oc3ccccc34)cc2)cc1 ; c1ccc(-c2ccc3c(oc4ccccc43)c2N(c2ccc3oc4ccccc4c3c2)c2cccc3ccccc23)cc1 ; C1=Cc2ccc(N(c3ccc4oc5ccccc5c4c3)c3c(-c4ccccc4)ccc4c3oc3ccccc34)cc2CC1] 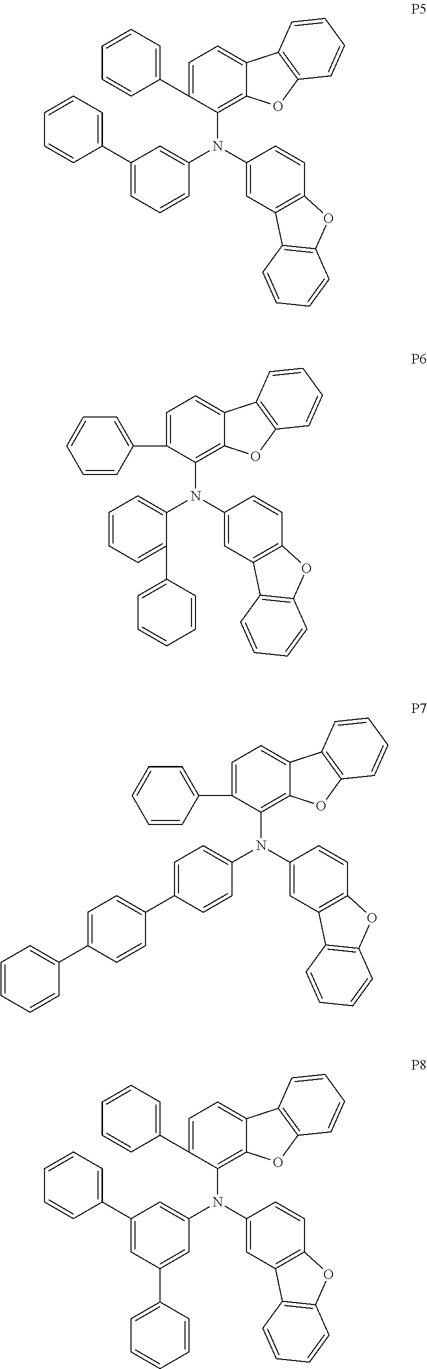 [SMILES: c1ccc(-c2cccc(N(c3ccc4oc5ccccc5c4c3)c3c(-c4ccccc4)ccc4c3oc3ccccc34)c2)cc1 ; c1ccc(-c2ccccc2N(c2ccc3oc4ccccc4c3c2)c2c(-c3ccccc3)ccc3c2oc2ccccc23)cc1 ; c1ccc(-c2ccc(-c3ccc(N(c4ccc5oc6ccccc6c5c4)c4c(-c5ccccc5)ccc5c4oc4ccccc45)cc3)cc2)cc1 ; c1ccc(-c2cc(-c3ccccc3)cc(N(c3ccc4oc5ccccc5c4c3)c3c(-c4ccccc4)ccc4c3oc3ccccc34)c2)cc1] 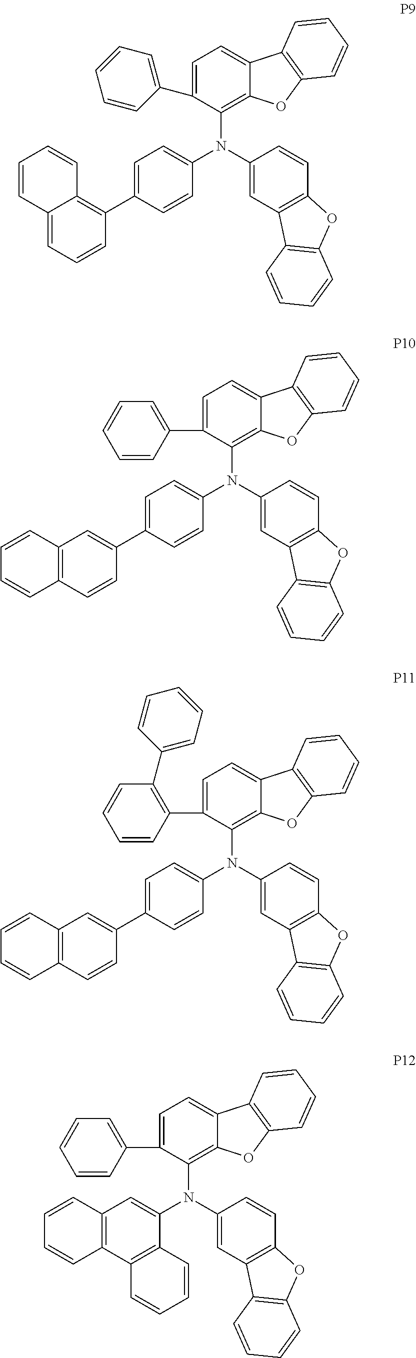 [SMILES: c1ccc(-c2ccc3c(oc4ccccc43)c2N(c2ccc(-c3cccc4ccccc34)cc2)c2ccc3oc4ccccc4c3c2)cc1 ; c1ccc(-c2ccc3c(oc4ccccc43)c2N(c2ccc(-c3ccc4ccccc4c3)cc2)c2ccc3oc4ccccc4c3c2)cc1 ; c1ccc(-c2ccccc2-c2ccc3c(oc4ccccc43)c2N(c2ccc(-c3ccc4ccccc4c3)cc2)c2ccc3oc4ccccc4c3c2)cc1 ; c1ccc(-c2ccc3c(oc4ccccc43)c2N(c2ccc3oc4ccccc4c3c2)c2cc3ccccc3c3ccccc23)cc1] 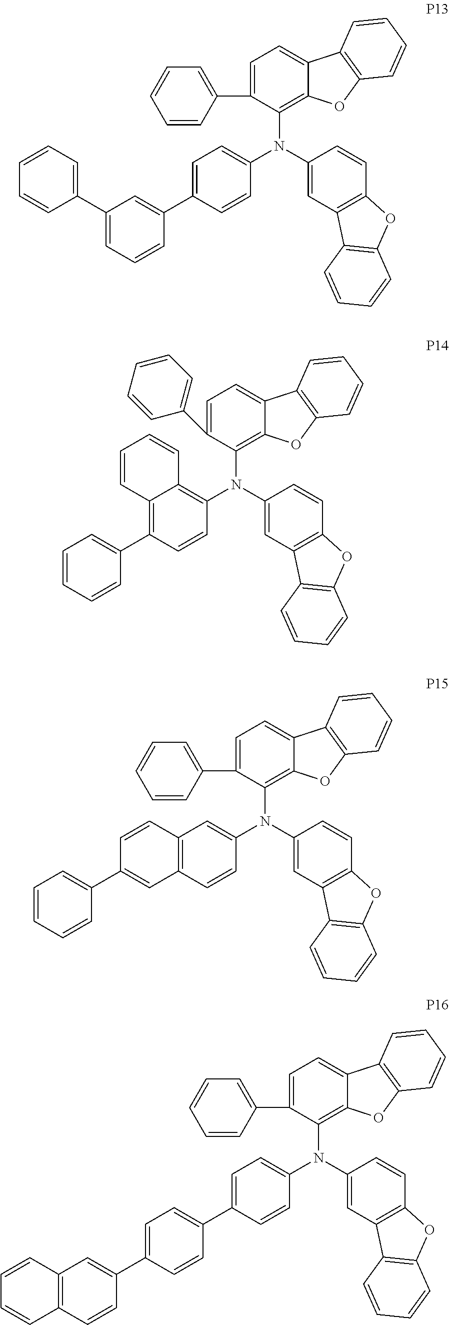 [SMILES: c1ccc(-c2cccc(-c3ccc(N(c4ccc5oc6ccccc6c5c4)c4c(-c5ccccc5)ccc5c4oc4ccccc45)cc3)c2)cc1 ; c1ccc(-c2ccc3c(oc4ccccc43)c2N(c2ccc3oc4ccccc4c3c2)c2ccc(-c3ccccc3)c3ccccc23)cc1 ; c1ccc(-c2ccc3cc(N(c4ccc5oc6ccccc6c5c4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc3oc4ccccc4c3c2)cc1] 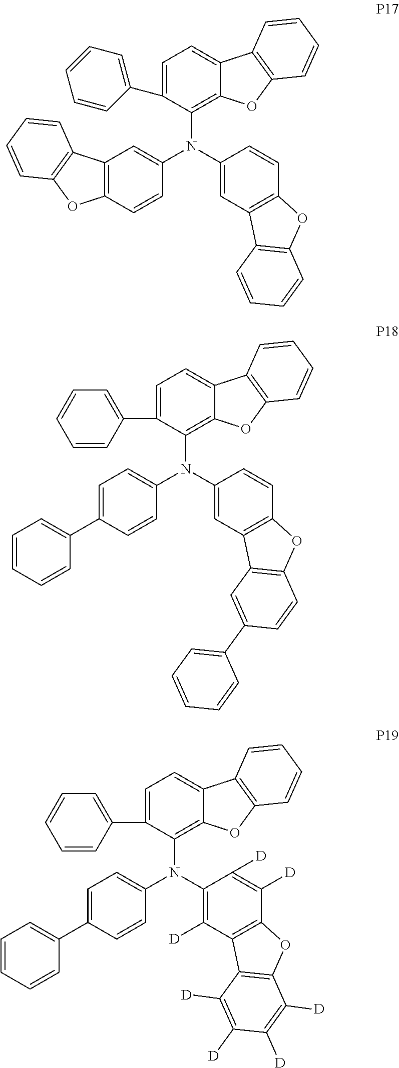 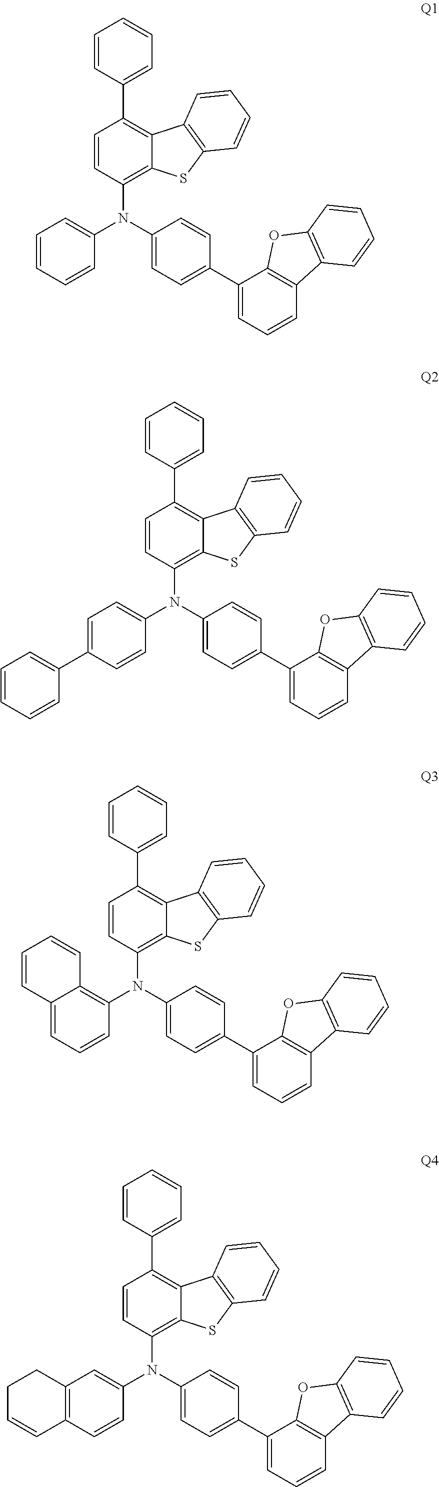 [SMILES: c1ccc(-c2ccc(N(c3ccccc3)c3ccc(-c4cccc5c4oc4ccccc45)cc3)c3sc4ccccc4c23)cc1 ; c1ccc(-c2ccc(N(c3ccc(-c4cccc5c4oc4ccccc45)cc3)c3ccc(-c4ccccc4)c4c3sc3ccccc34)cc2)cc1 ; c1ccc(-c2ccc(N(c3ccc(-c4cccc5c4oc4ccccc45)cc3)c3cccc4ccccc34)c3sc4ccccc4c23)cc1 ; C1=Cc2ccc(N(c3ccc(-c4cccc5c4oc4ccccc45)cc3)c3ccc(-c4ccccc4)c4c3sc3ccccc34)cc2CC1] 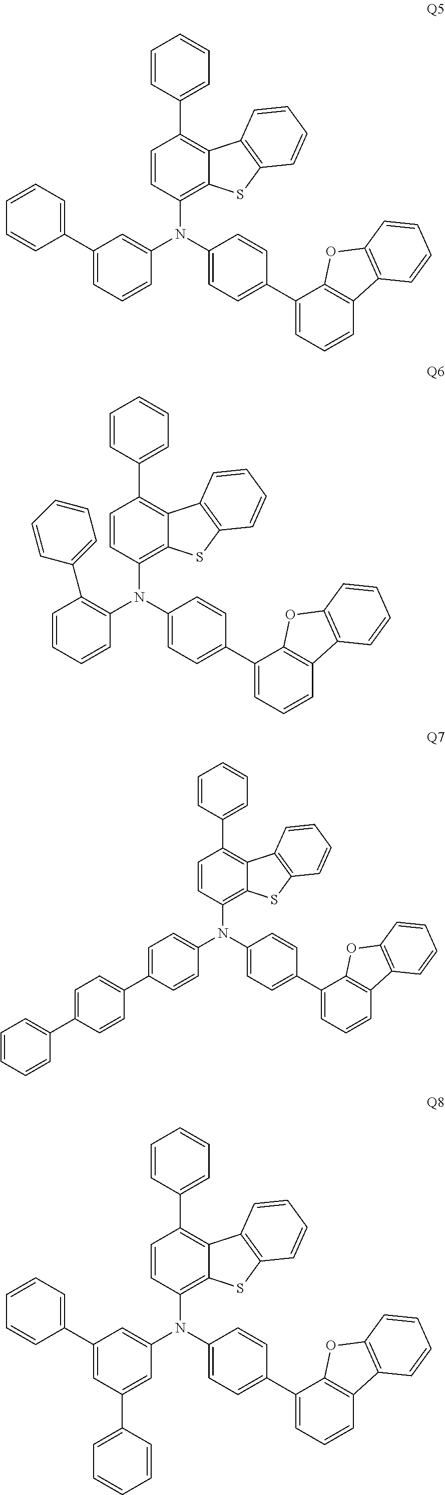 [SMILES: c1ccc(-c2cccc(N(c3ccc(-c4cccc5c4oc4ccccc45)cc3)c3ccc(-c4ccccc4)c4c3sc3ccccc34)c2)cc1 ; c1ccc(-c2ccccc2N(c2ccc(-c3cccc4c3oc3ccccc34)cc2)c2ccc(-c3ccccc3)c3c2sc2ccccc23)cc1 ; c1ccc(-c2ccc(-c3ccc(N(c4ccc(-c5cccc6c5oc5ccccc56)cc4)c4ccc(-c5ccccc5)c5c4sc4ccccc45)cc3)cc2)cc1 ; c1ccc(-c2cc(-c3ccccc3)cc(N(c3ccc(-c4cccc5c4oc4ccccc45)cc3)c3ccc(-c4ccccc4)c4c3sc3ccccc34)c2)cc1] 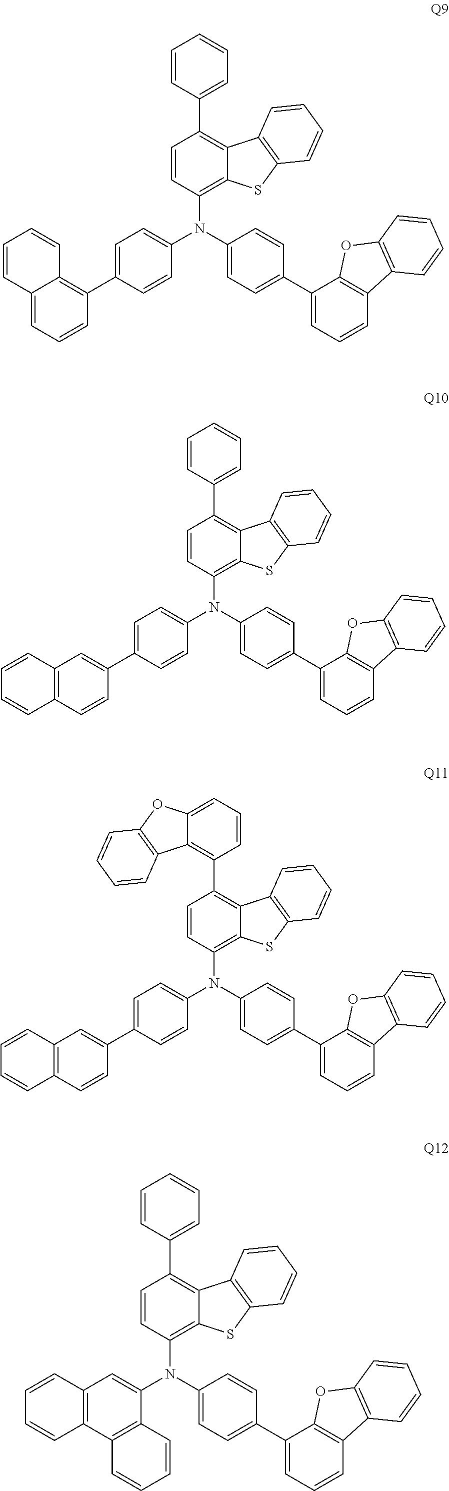 [SMILES: c1ccc(-c2ccc(N(c3ccc(-c4cccc5ccccc45)cc3)c3ccc(-c4cccc5c4oc4ccccc45)cc3)c3sc4ccccc4c23)cc1 ; c1ccc(-c2ccc(N(c3ccc(-c4ccc5ccccc5c4)cc3)c3ccc(-c4cccc5c4oc4ccccc45)cc3)c3sc4ccccc4c23)cc1 ; c1ccc2cc(-c3ccc(N(c4ccc(-c5cccc6c5oc5ccccc56)cc4)c4ccc(-c5cccc6oc7ccccc7c56)c5c4sc4ccccc45)cc3)ccc2c1 ; c1ccc(-c2ccc(N(c3ccc(-c4cccc5c4oc4ccccc45)cc3)c3cc4ccccc4c4ccccc34)c3sc4ccccc4c23)cc1] 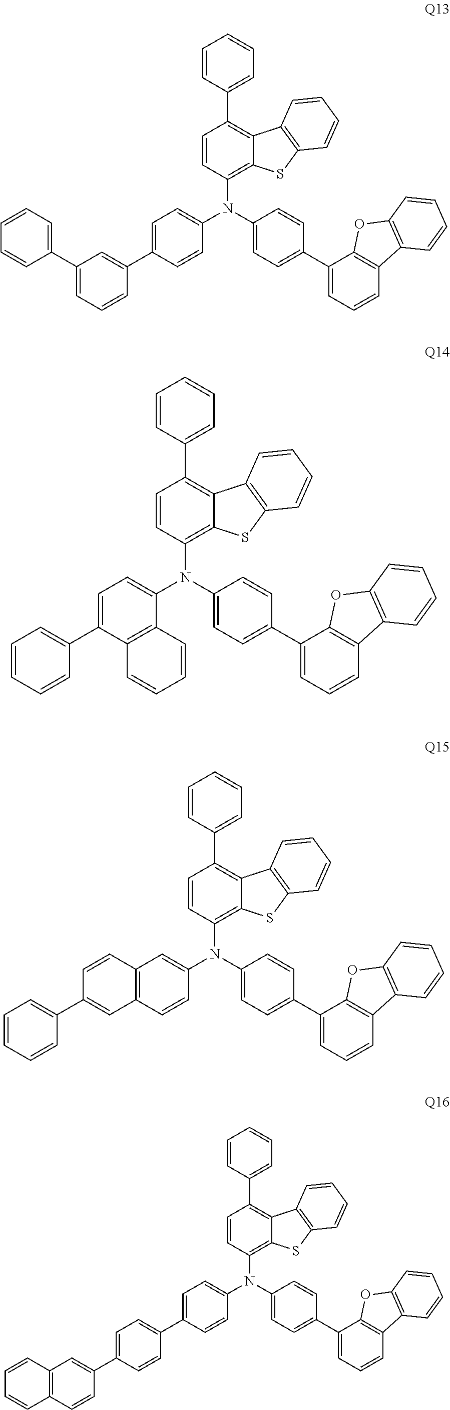 [SMILES: c1ccc(-c2cccc(-c3ccc(N(c4ccc(-c5cccc6c5oc5ccccc56)cc4)c4ccc(-c5ccccc5)c5c4sc4ccccc45)cc3)c2)cc1 ; c1ccc(-c2ccc(N(c3ccc(-c4cccc5c4oc4ccccc45)cc3)c3ccc(-c4ccccc4)c4c3sc3ccccc34)c3ccccc23)cc1 ; c1ccc(-c2ccc3cc(N(c4ccc(-c5cccc6c5oc5ccccc56)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4cccc5c4oc4ccccc45)cc3)c3sc4ccccc4c23)cc1] 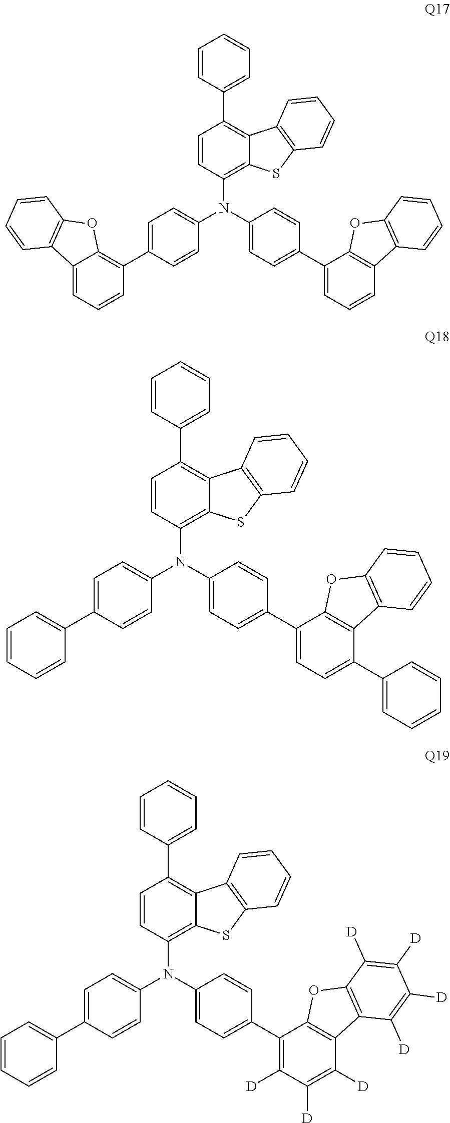 [SMILES: c1ccc(-c2ccc(N(c3ccc(-c4cccc5c4oc4ccccc45)cc3)c3ccc(-c4cccc5c4oc4ccccc45)cc3)c3sc4ccccc4c23)cc1 ; c1ccc(-c2ccc(N(c3ccc(-c4ccc(-c5ccccc5)c5c4oc4ccccc45)cc3)c3ccc(-c4ccccc4)c4c3sc3ccccc34)cc2)cc1 ; [2H]c1c([2H])c([2H])c2c(oc3c(-c4ccc(N(c5ccc(-c6ccccc6)cc5)c5ccc(-c6ccccc6)c6c5sc5ccccc56)cc4)c([2H])c([2H])c([2H])c32)c1[2H]] 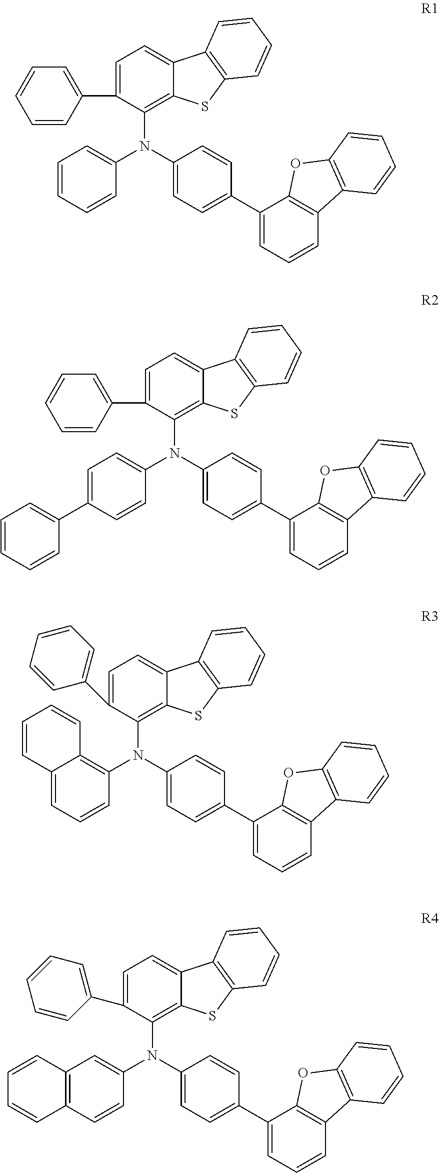 [SMILES: c1ccc(-c2ccc3c(sc4ccccc43)c2N(c2ccccc2)c2ccc(-c3cccc4c3oc3ccccc34)cc2)cc1 ; c1ccc(-c2ccc(N(c3ccc(-c4cccc5c4oc4ccccc45)cc3)c3c(-c4ccccc4)ccc4c3sc3ccccc34)cc2)cc1 ; c1ccc(-c2ccc3c(sc4ccccc43)c2N(c2ccc(-c3cccc4c3oc3ccccc34)cc2)c2cccc3ccccc23)cc1 ; c1ccc(-c2ccc3c(sc4ccccc43)c2N(c2ccc(-c3cccc4c3oc3ccccc34)cc2)c2ccc3ccccc3c2)cc1] 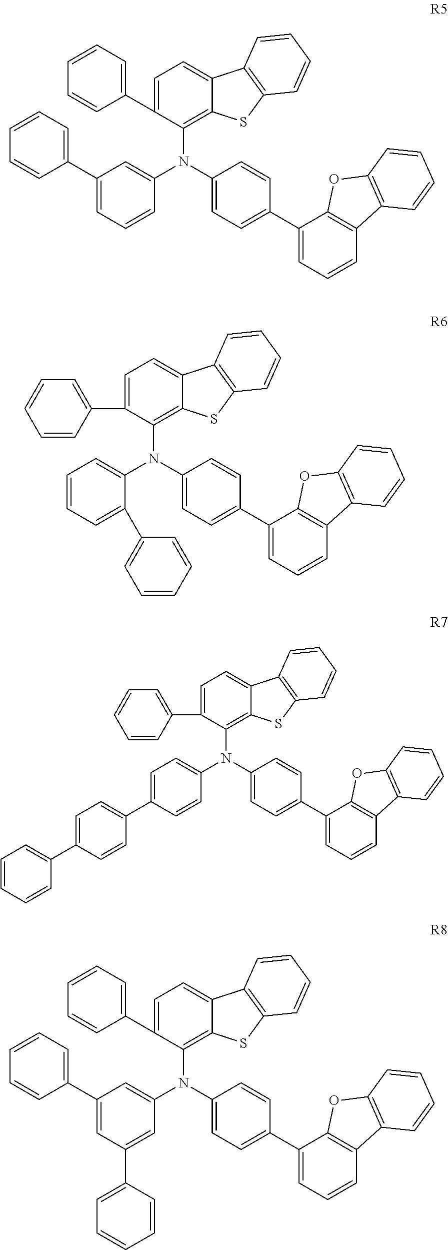 [SMILES: c1ccc(-c2cccc(N(c3ccc(-c4cccc5c4oc4ccccc45)cc3)c3c(-c4ccccc4)ccc4c3sc3ccccc34)c2)cc1 ; c1ccc(-c2ccccc2N(c2ccc(-c3cccc4c3oc3ccccc34)cc2)c2c(-c3ccccc3)ccc3c2sc2ccccc23)cc1 ; c1ccc(-c2ccc(-c3ccc(N(c4ccc(-c5cccc6c5oc5ccccc56)cc4)c4c(-c5ccccc5)ccc5c4sc4ccccc45)cc3)cc2)cc1 ; c1ccc(-c2cc(-c3ccccc3)cc(N(c3ccc(-c4cccc5c4oc4ccccc45)cc3)c3c(-c4ccccc4)ccc4c3sc3ccccc34)c2)cc1] 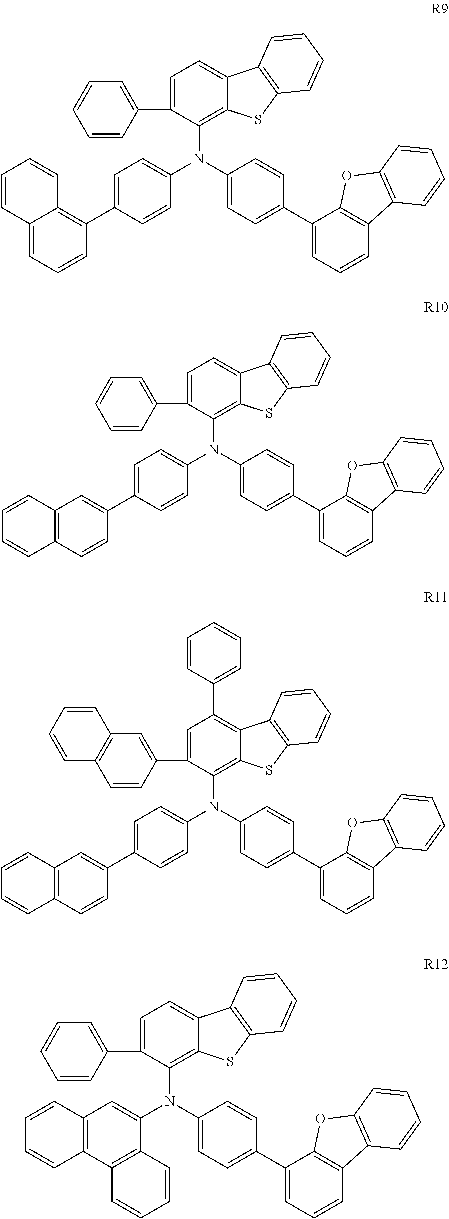 [SMILES: c1ccc(-c2ccc3c(sc4ccccc43)c2N(c2ccc(-c3cccc4ccccc34)cc2)c2ccc(-c3cccc4c3oc3ccccc34)cc2)cc1 ; c1ccc(-c2ccc3c(sc4ccccc43)c2N(c2ccc(-c3ccc4ccccc4c3)cc2)c2ccc(-c3cccc4c3oc3ccccc34)cc2)cc1 ; c1ccc(-c2cc(-c3ccc4ccccc4c3)c(N(c3ccc(-c4ccc5ccccc5c4)cc3)c3ccc(-c4cccc5c4oc4ccccc45)cc3)c3sc4ccccc4c23)cc1 ; c1ccc(-c2ccc3c(sc4ccccc43)c2N(c2ccc(-c3cccc4c3oc3ccccc34)cc2)c2cc3ccccc3c3ccccc23)cc1] 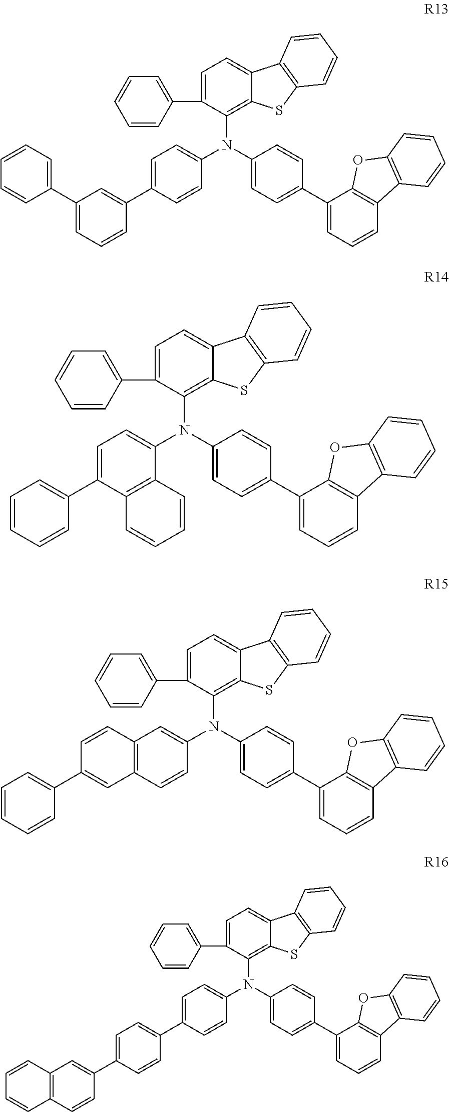 [SMILES: c1ccc(-c2cccc(-c3ccc(N(c4ccc(-c5cccc6c5oc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3cccc4c3oc3ccccc34)cc2)c2ccc(-c3ccccc3)c3ccccc23)cc1 ; c1ccc(-c2ccc3cc(N(c4ccc(-c5cccc6c5oc5ccccc56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3cccc4c3oc3ccccc34)cc2)cc1] 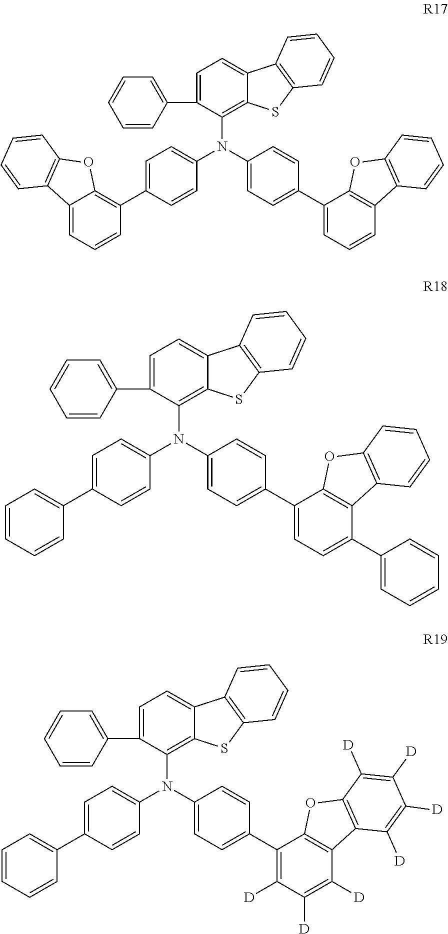 [SMILES: c1ccc(-c2ccc3c(sc4ccccc43)c2N(c2ccc(-c3cccc4c3oc3ccccc34)cc2)c2ccc(-c3cccc4c3oc3ccccc34)cc2)cc1 ; c1ccc(-c2ccc(N(c3ccc(-c4ccc(-c5ccccc5)c5c4oc4ccccc45)cc3)c3c(-c4ccccc4)ccc4c3sc3ccccc34)cc2)cc1 ; [2H]c1c([2H])c([2H])c2c(oc3c(-c4ccc(N(c5ccc(-c6ccccc6)cc5)c5c(-c6ccccc6)ccc6c5sc5ccccc56)cc4)c([2H])c([2H])c([2H])c32)c1[2H]] 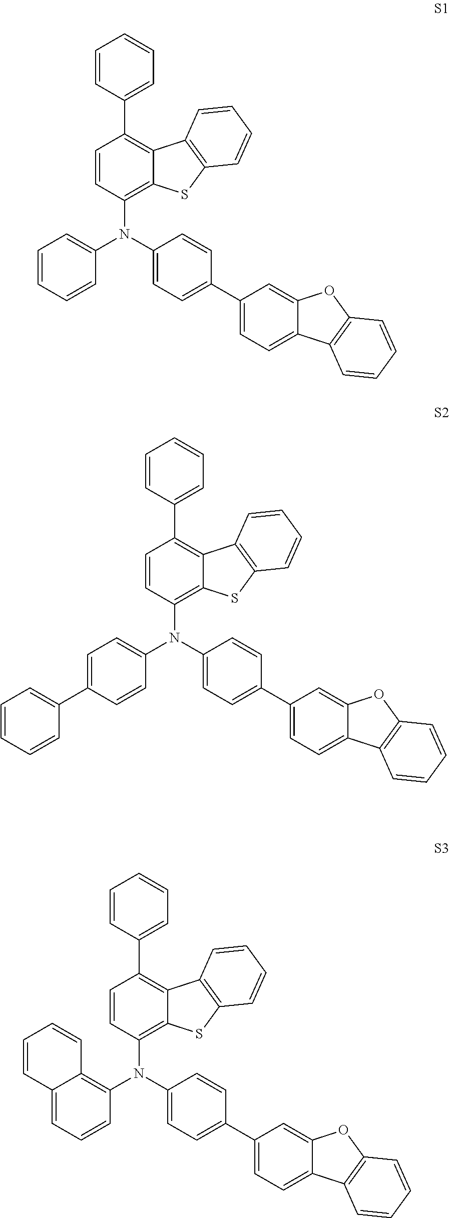 [SMILES: c1ccc(-c2ccc(N(c3ccccc3)c3ccc(-c4ccc5c(c4)oc4ccccc45)cc3)c3sc4ccccc4c23)cc1 ; c1ccc(-c2ccc(N(c3ccc(-c4ccc5c(c4)oc4ccccc45)cc3)c3ccc(-c4ccccc4)c4c3sc3ccccc34)cc2)cc1 ; c1ccc(-c2ccc(N(c3ccc(-c4ccc5c(c4)oc4ccccc45)cc3)c3cccc4ccccc34)c3sc4ccccc4c23)cc1] 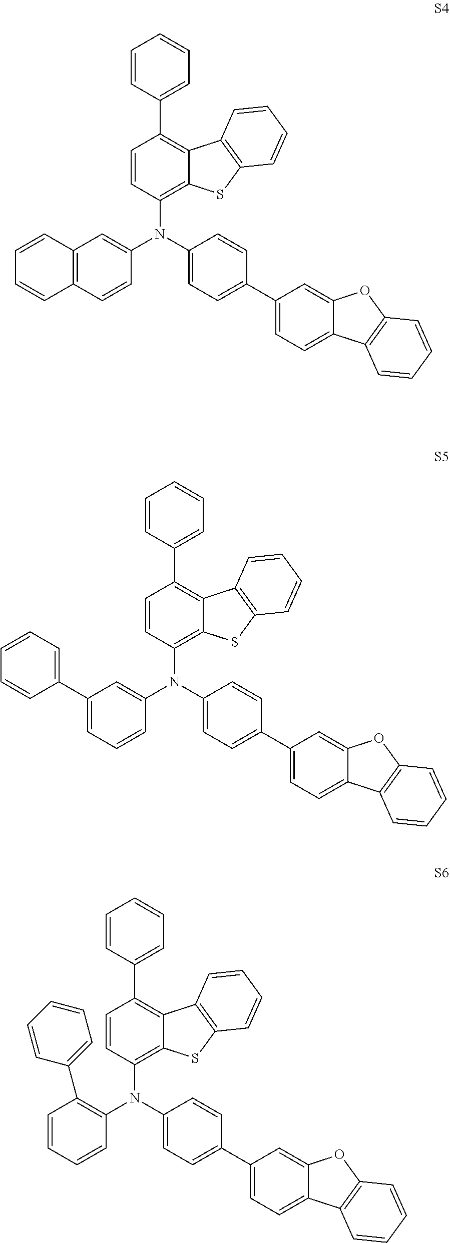 [SMILES: c1ccc(-c2ccc(N(c3ccc(-c4ccc5c(c4)oc4ccccc45)cc3)c3ccc4ccccc4c3)c3sc4ccccc4c23)cc1 ; c1ccc(-c2cccc(N(c3ccc(-c4ccc5c(c4)oc4ccccc45)cc3)c3ccc(-c4ccccc4)c4c3sc3ccccc34)c2)cc1 ; c1ccc(-c2ccccc2N(c2ccc(-c3ccc4c(c3)oc3ccccc34)cc2)c2ccc(-c3ccccc3)c3c2sc2ccccc23)cc1] 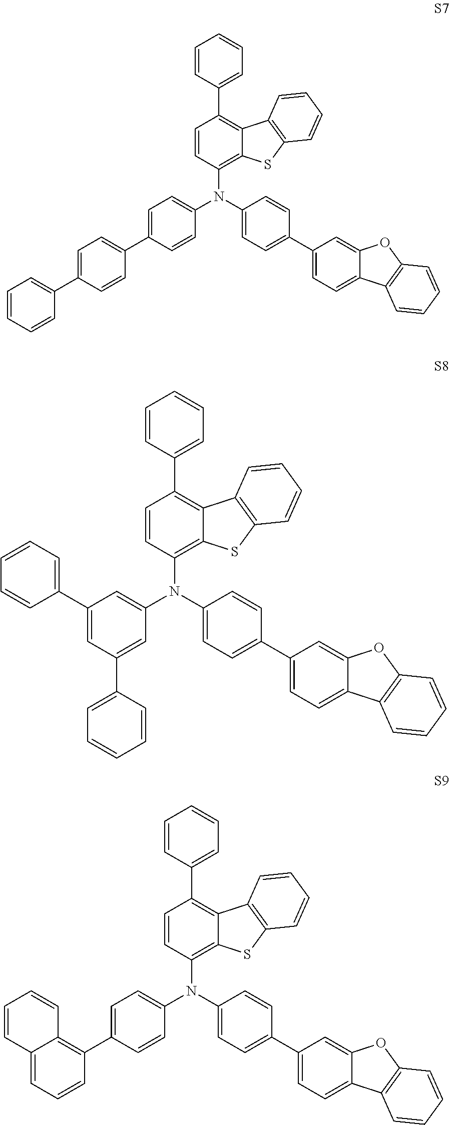 [SMILES: c1ccc(-c2ccc(-c3ccc(N(c4ccc(-c5ccc6c(c5)oc5ccccc56)cc4)c4ccc(-c5ccccc5)c5c4sc4ccccc45)cc3)cc2)cc1 ; c1ccc(-c2cc(-c3ccccc3)cc(N(c3ccc(-c4ccc5c(c4)oc4ccccc45)cc3)c3ccc(-c4ccccc4)c4c3sc3ccccc34)c2)cc1 ; c1ccc(-c2ccc(N(c3ccc(-c4ccc5c(c4)oc4ccccc45)cc3)c3ccc(-c4cccc5ccccc45)cc3)c3sc4ccccc4c23)cc1] 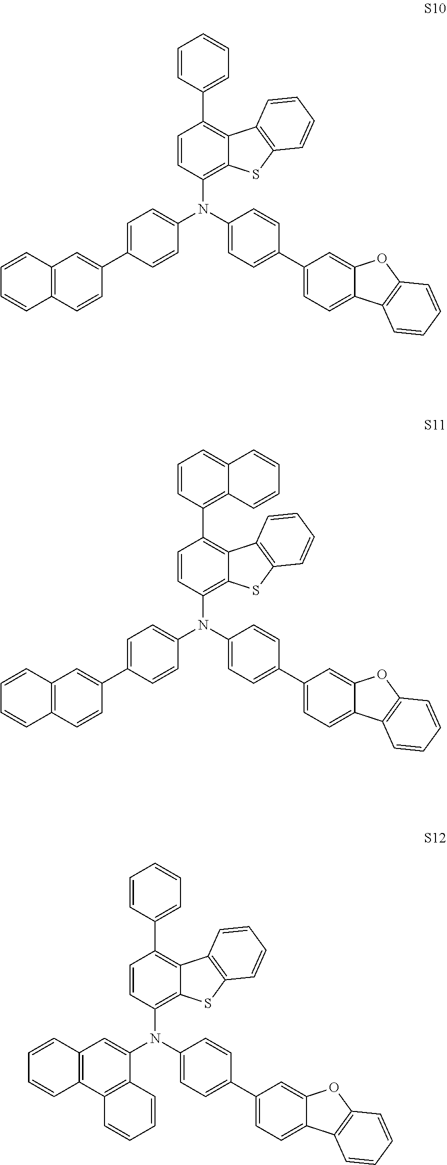 [SMILES: c1ccc(-c2ccc(N(c3ccc(-c4ccc5ccccc5c4)cc3)c3ccc(-c4ccc5c(c4)oc4ccccc45)cc3)c3sc4ccccc4c23)cc1 ; c1ccc2cc(-c3ccc(N(c4ccc(-c5ccc6c(c5)oc5ccccc56)cc4)c4ccc(-c5cccc6ccccc56)c5c4sc4ccccc45)cc3)ccc2c1 ; c1ccc(-c2ccc(N(c3ccc(-c4ccc5c(c4)oc4ccccc45)cc3)c3cc4ccccc4c4ccccc34)c3sc4ccccc4c23)cc1] 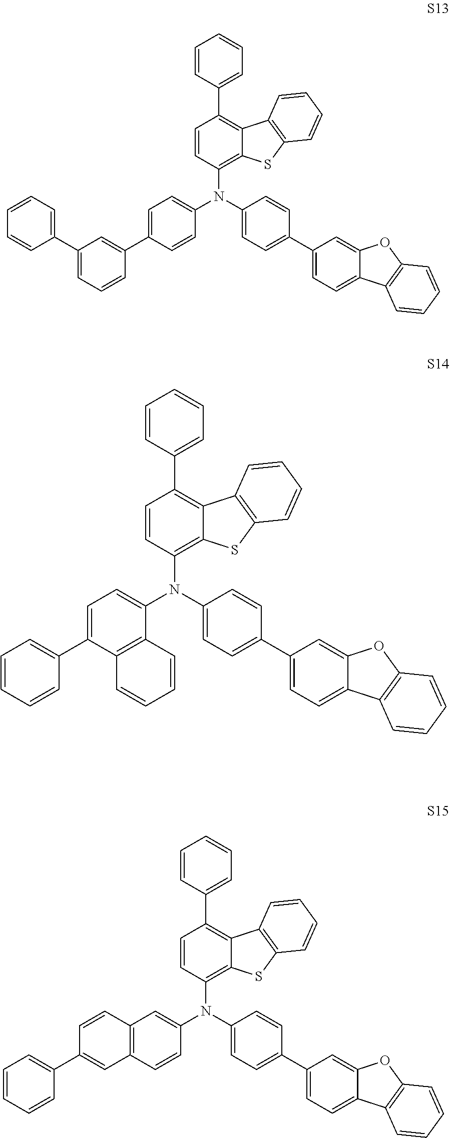 [SMILES: c1ccc(-c2cccc(-c3ccc(N(c4ccc(-c5ccc6c(c5)oc5ccccc56)cc4)c4ccc(-c5ccccc5)c5c4sc4ccccc45)cc3)c2)cc1 ; c1ccc(-c2ccc(N(c3ccc(-c4ccc5c(c4)oc4ccccc45)cc3)c3ccc(-c4ccccc4)c4c3sc3ccccc34)c3ccccc23)cc1 ; c1ccc(-c2ccc3cc(N(c4ccc(-c5ccc6c(c5)oc5ccccc56)cc4)c4ccc(-c5ccccc5)c5c4sc4ccccc45)ccc3c2)cc1] 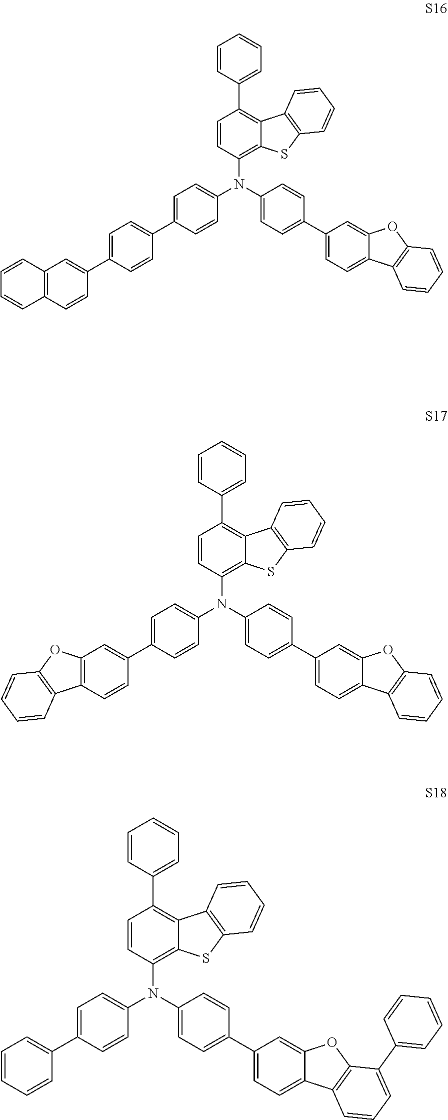 [SMILES: c1ccc(-c2ccc(N(c3ccc(-c4ccc(-c5ccc6ccccc6c5)cc4)cc3)c3ccc(-c4ccc5c(c4)oc4ccccc45)cc3)c3sc4ccccc4c23)cc1 ; c1ccc(-c2ccc(N(c3ccc(-c4ccc5c(c4)oc4ccccc45)cc3)c3ccc(-c4ccc5c(c4)oc4ccccc45)cc3)c3sc4ccccc4c23)cc1 ; c1ccc(-c2ccc(N(c3ccc(-c4ccc5c(c4)oc4c(-c6ccccc6)cccc45)cc3)c3ccc(-c4ccccc4)c4c3sc3ccccc34)cc2)cc1] 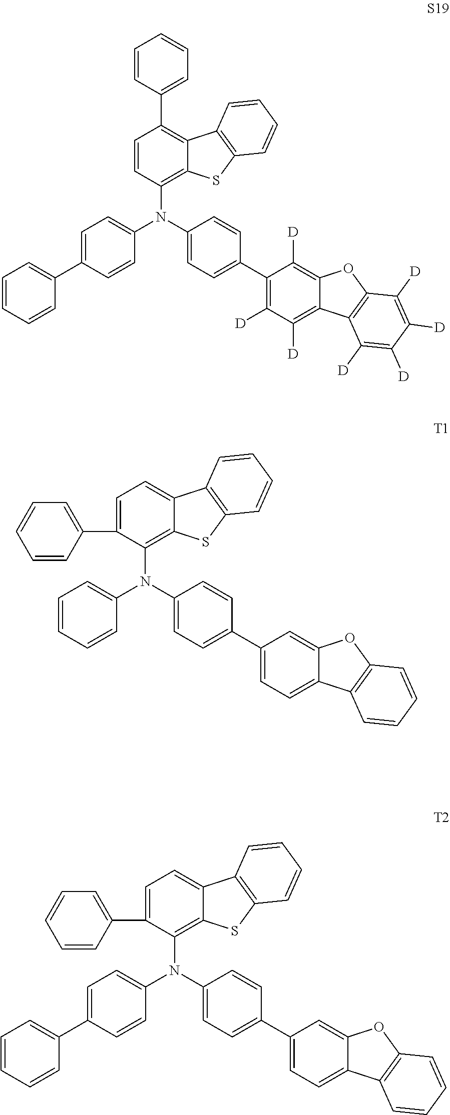 [SMILES: [2H]c1c([2H])c([2H])c2c(oc3c([2H])c(-c4ccc(N(c5ccc(-c6ccccc6)cc5)c5ccc(-c6ccccc6)c6c5sc5ccccc56)cc4)c([2H])c([2H])c32)c1[2H] ; c1ccc(-c2ccc3c(sc4ccccc43)c2N(c2ccccc2)c2ccc(-c3ccc4c(c3)oc3ccccc34)cc2)cc1 ; c1ccc(-c2ccc(N(c3ccc(-c4ccc5c(c4)oc4ccccc45)cc3)c3c(-c4ccccc4)ccc4c3sc3ccccc34)cc2)cc1] 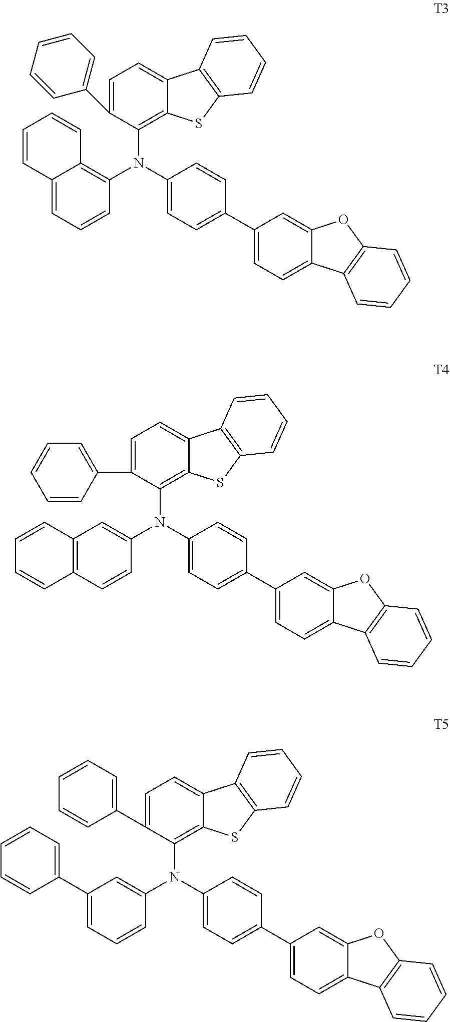 [SMILES: c1ccc(-c2ccc3c(sc4ccccc43)c2N(c2ccc(-c3ccc4c(c3)oc3ccccc34)cc2)c2cccc3ccccc23)cc1 ; c1ccc(-c2ccc3c(sc4ccccc43)c2N(c2ccc(-c3ccc4c(c3)oc3ccccc34)cc2)c2ccc3ccccc3c2)cc1 ; c1ccc(-c2cccc(N(c3ccc(-c4ccc5c(c4)oc4ccccc45)cc3)c3c(-c4ccccc4)ccc4c3sc3ccccc34)c2)cc1] 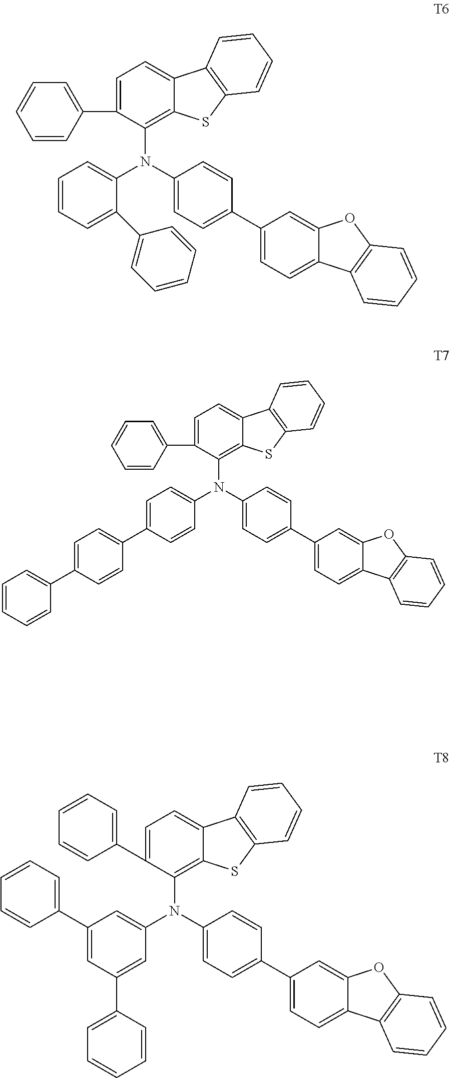 [SMILES: c1ccc(-c2ccccc2N(c2ccc(-c3ccc4c(c3)oc3ccccc34)cc2)c2c(-c3ccccc3)ccc3c2sc2ccccc23)cc1 ; c1ccc(-c2ccc(-c3ccc(N(c4ccc(-c5ccc6c(c5)oc5ccccc56)cc4)c4c(-c5ccccc5)ccc5c4sc4ccccc45)cc3)cc2)cc1 ; c1ccc(-c2cc(-c3ccccc3)cc(N(c3ccc(-c4ccc5c(c4)oc4ccccc45)cc3)c3c(-c4ccccc4)ccc4c3sc3ccccc34)c2)cc1] 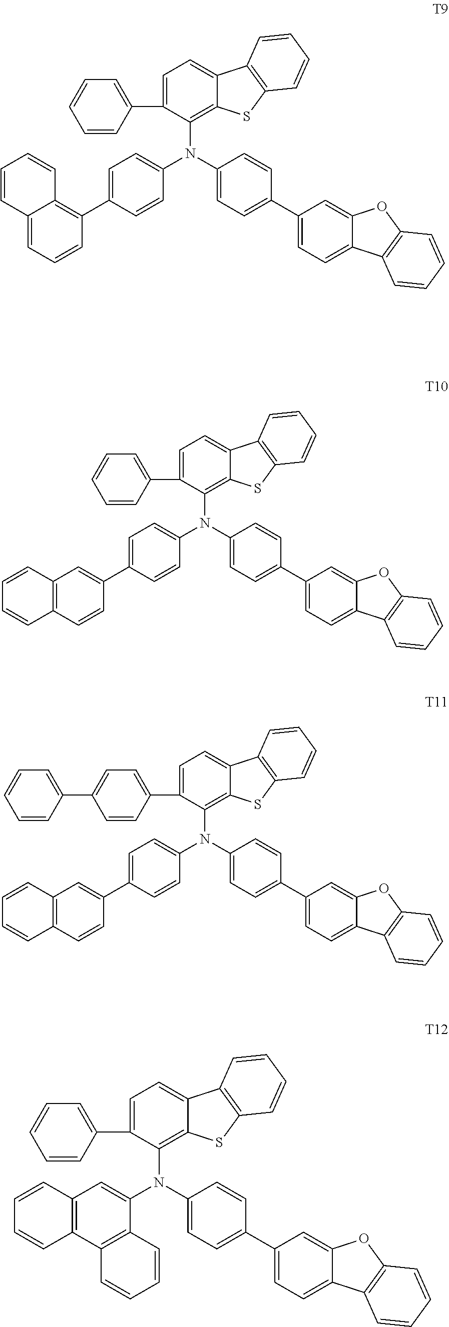 [SMILES: c1ccc(-c2ccc3c(sc4ccccc43)c2N(c2ccc(-c3ccc4c(c3)oc3ccccc34)cc2)c2ccc(-c3cccc4ccccc34)cc2)cc1 ; c1ccc(-c2ccc3c(sc4ccccc43)c2N(c2ccc(-c3ccc4ccccc4c3)cc2)c2ccc(-c3ccc4c(c3)oc3ccccc34)cc2)cc1 ; c1ccc(-c2ccc(-c3ccc4c(sc5ccccc54)c3N(c3ccc(-c4ccc5ccccc5c4)cc3)c3ccc(-c4ccc5c(c4)oc4ccccc45)cc3)cc2)cc1 ; c1ccc(-c2ccc3c(sc4ccccc43)c2N(c2ccc(-c3ccc4c(c3)oc3ccccc34)cc2)c2cc3ccccc3c3ccccc23)cc1] 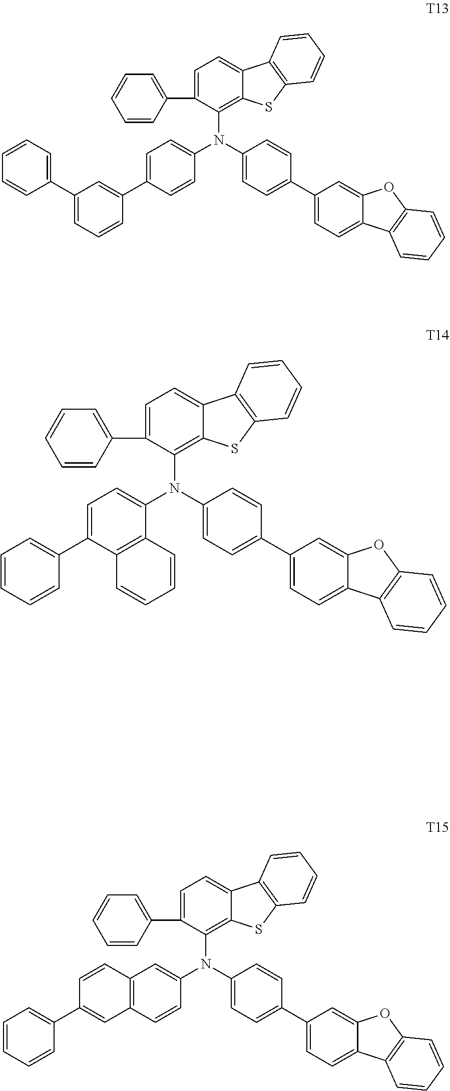 [SMILES: c1ccc(-c2cccc(-c3ccc(N(c4ccc(-c5ccc6c(c5)oc5ccccc56)cc4)c4c(-c5ccccc5)ccc5c4sc4ccccc45)cc3)c2)cc1 ; c1ccc(-c2ccc3c(sc4ccccc43)c2N(c2ccc(-c3ccc4c(c3)oc3ccccc34)cc2)c2ccc(-c3ccccc3)c3ccccc23)cc1 ; c1ccc(-c2ccc3cc(N(c4ccc(-c5ccc6c(c5)oc5ccccc56)cc4)c4c(-c5ccccc5)ccc5c4sc4ccccc45)ccc3c2)cc1] 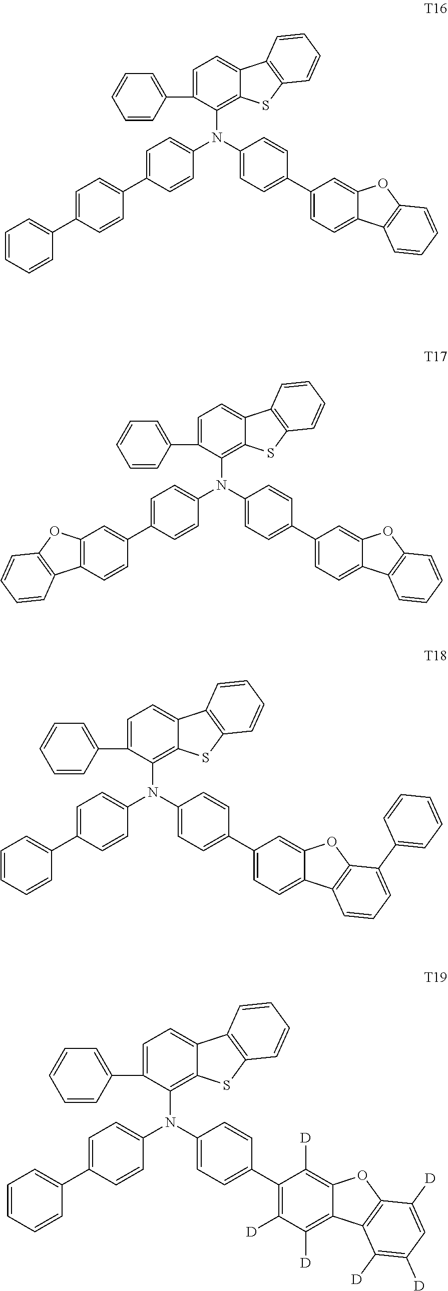 [SMILES: c1ccc(-c2ccc(-c3ccc(N(c4ccc(-c5ccc6c(c5)oc5ccccc56)cc4)c4c(-c5ccccc5)ccc5c4sc4ccccc45)cc3)cc2)cc1 ; c1ccc(-c2ccc3c(sc4ccccc43)c2N(c2ccc(-c3ccc4c(c3)oc3ccccc34)cc2)c2ccc(-c3ccc4c(c3)oc3ccccc34)cc2)cc1 ; c1ccc(-c2ccc(N(c3ccc(-c4ccc5c(c4)oc4c(-c6ccccc6)cccc45)cc3)c3c(-c4ccccc4)ccc4c3sc3ccccc34)cc2)cc1 ; [2H]c1cc([2H])c2oc3c([2H])c(-c4ccc(N(c5ccc(-c6ccccc6)cc5)c5c(-c6ccccc6)ccc6c5sc5ccccc56)cc4)c([2H])c([2H])c3c2c1[2H]] 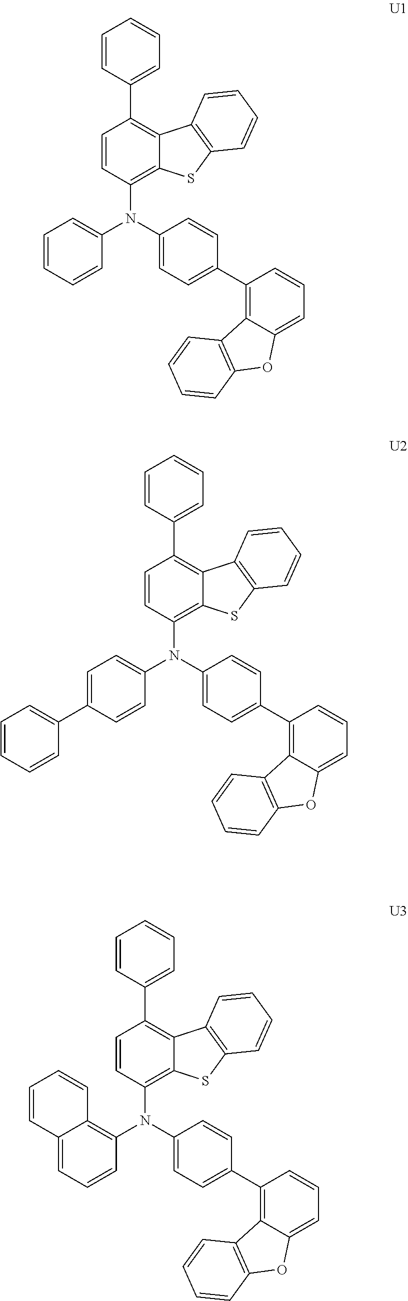 [SMILES: c1ccc(-c2ccc(N(c3ccccc3)c3ccc(-c4cccc5oc6ccccc6c45)cc3)c3sc4ccccc4c23)cc1 ; c1ccc(-c2ccc(N(c3ccc(-c4cccc5oc6ccccc6c45)cc3)c3ccc(-c4ccccc4)c4c3sc3ccccc34)cc2)cc1 ; c1ccc(-c2ccc(N(c3ccc(-c4cccc5oc6ccccc6c45)cc3)c3cccc4ccccc34)c3sc4ccccc4c23)cc1] 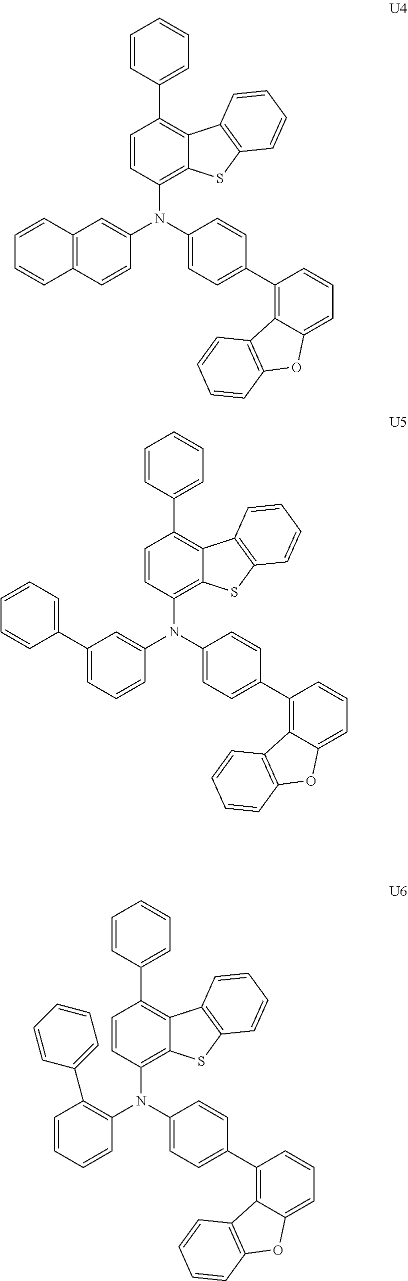 [SMILES: c1ccc(-c2ccc(N(c3ccc(-c4cccc5oc6ccccc6c45)cc3)c3ccc4ccccc4c3)c3sc4ccccc4c23)cc1 ; c1ccc(-c2cccc(N(c3ccc(-c4cccc5oc6ccccc6c45)cc3)c3ccc(-c4ccccc4)c4c3sc3ccccc34)c2)cc1 ; c1ccc(-c2ccccc2N(c2ccc(-c3cccc4oc5ccccc5c34)cc2)c2ccc(-c3ccccc3)c3c2sc2ccccc23)cc1] 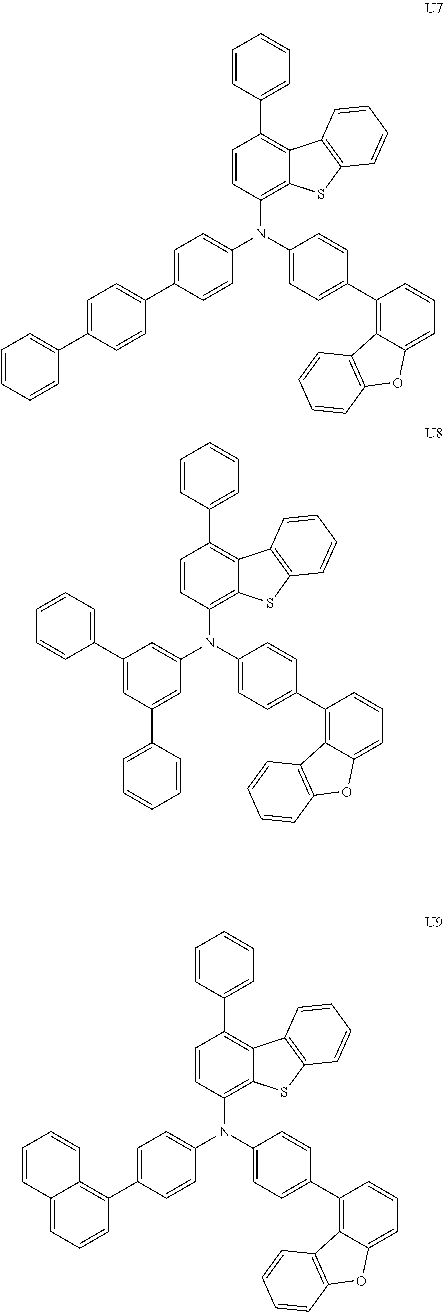 [SMILES: c1ccc(-c2ccc(-c3ccc(N(c4ccc(-c5cccc6oc7ccccc7c56)cc4)c4ccc(-c5ccccc5)c5c4sc4ccccc45)cc3)cc2)cc1 ; c1ccc(-c2cc(-c3ccccc3)cc(N(c3ccc(-c4cccc5oc6ccccc6c45)cc3)c3ccc(-c4ccccc4)c4c3sc3ccccc34)c2)cc1 ; c1ccc(-c2ccc(N(c3ccc(-c4cccc5ccccc45)cc3)c3ccc(-c4cccc5oc6ccccc6c45)cc3)c3sc4ccccc4c23)cc1] 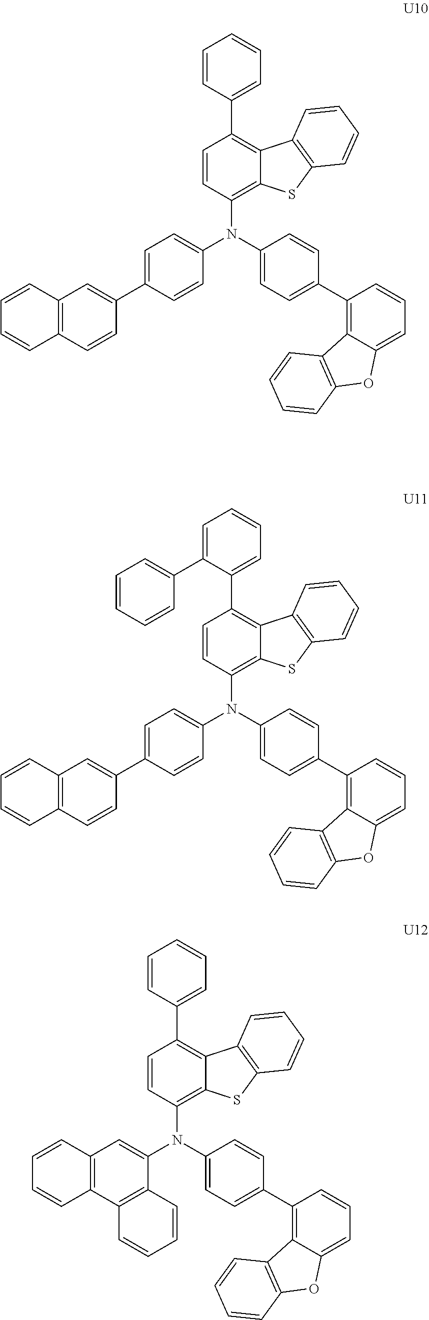 [SMILES: c1ccc(-c2ccc(N(c3ccc(-c4ccc5ccccc5c4)cc3)c3ccc(-c4cccc5oc6ccccc6c45)cc3)c3sc4ccccc4c23)cc1 ; c1ccc(-c2ccccc2-c2ccc(N(c3ccc(-c4ccc5ccccc5c4)cc3)c3ccc(-c4cccc5oc6ccccc6c45)cc3)c3sc4ccccc4c23)cc1 ; c1ccc(-c2ccc(N(c3ccc(-c4cccc5oc6ccccc6c45)cc3)c3cc4ccccc4c4ccccc34)c3sc4ccccc4c23)cc1] 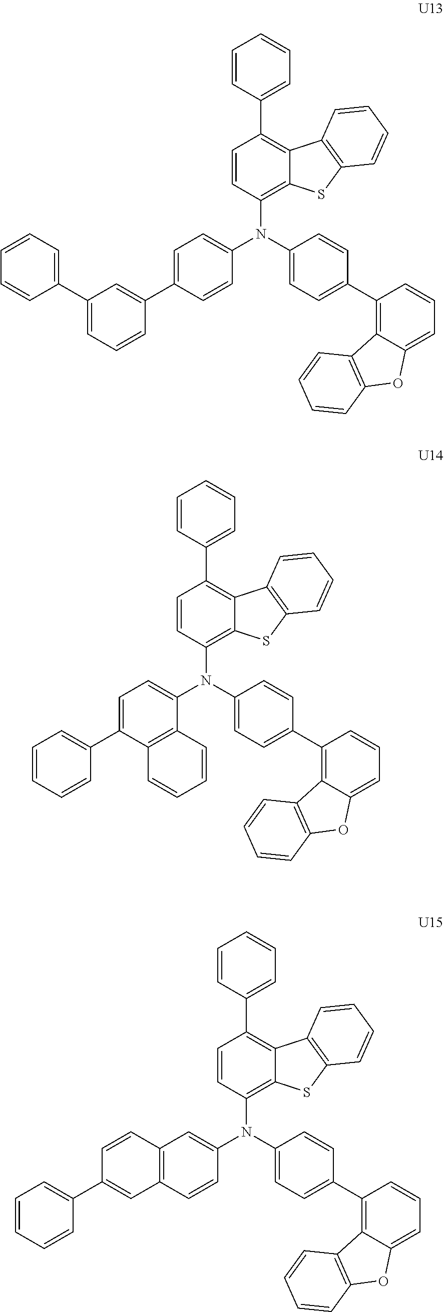 [SMILES: c1ccc(-c2cccc(-c3ccc(N(c4ccc(-c5cccc6oc7ccccc7c56)cc4)c4ccc(-c5ccccc5)c5c4sc4ccccc45)cc3)c2)cc1 ; c1ccc(-c2ccc(N(c3ccc(-c4cccc5oc6ccccc6c45)cc3)c3ccc(-c4ccccc4)c4c3sc3ccccc34)c3ccccc23)cc1 ; c1ccc(-c2ccc3cc(N(c4ccc(-c5cccc6oc7ccccc7c56)cc4)c4ccc(-c5ccccc5)c5c4sc4ccccc45)ccc3c2)cc1] 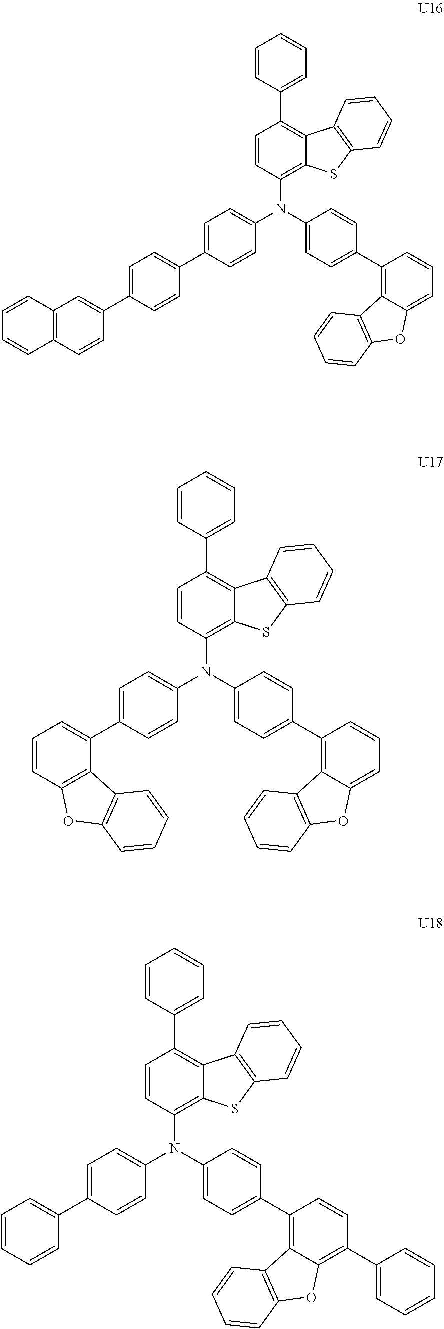 [SMILES: c1ccc(-c2ccc(N(c3ccc(-c4ccc(-c5ccc6ccccc6c5)cc4)cc3)c3ccc(-c4cccc5oc6ccccc6c45)cc3)c3sc4ccccc4c23)cc1 ; c1ccc(-c2ccc(N(c3ccc(-c4cccc5oc6ccccc6c45)cc3)c3ccc(-c4cccc5oc6ccccc6c45)cc3)c3sc4ccccc4c23)cc1 ; c1ccc(-c2ccc(N(c3ccc(-c4ccc(-c5ccccc5)c5oc6ccccc6c45)cc3)c3ccc(-c4ccccc4)c4c3sc3ccccc34)cc2)cc1] 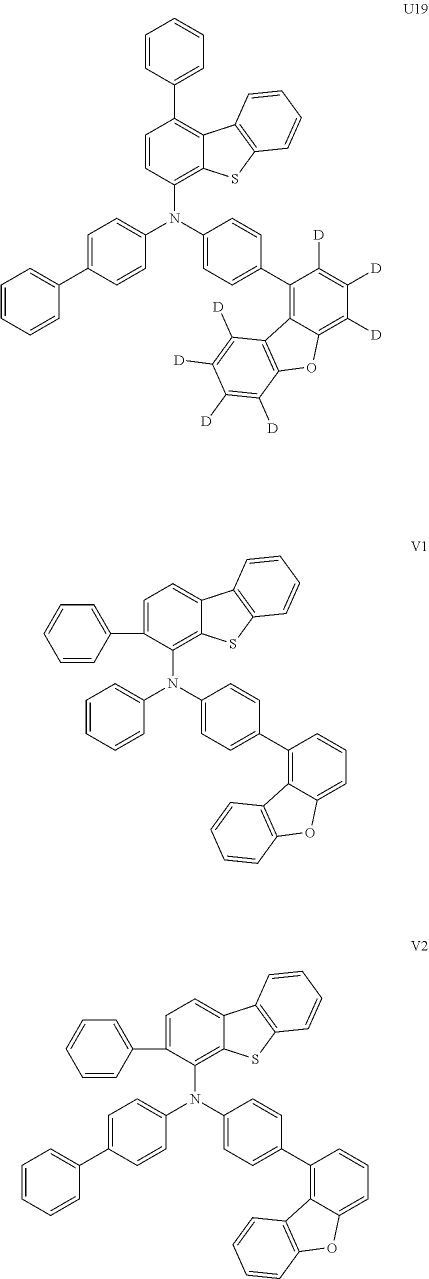 [SMILES: [2H]c1c([2H])c([2H])c2c(oc3c([2H])c([2H])c([2H])c(-c4ccc(N(c5ccc(-c6ccccc6)cc5)c5ccc(-c6ccccc6)c6c5sc5ccccc56)cc4)c32)c1[2H] ; c1ccc(-c2ccc3c(sc4ccccc43)c2N(c2ccccc2)c2ccc(-c3cccc4oc5ccccc5c34)cc2)cc1 ; c1ccc(-c2ccc(N(c3ccc(-c4cccc5oc6ccccc6c45)cc3)c3c(-c4ccccc4)ccc4c3sc3ccccc34)cc2)cc1] 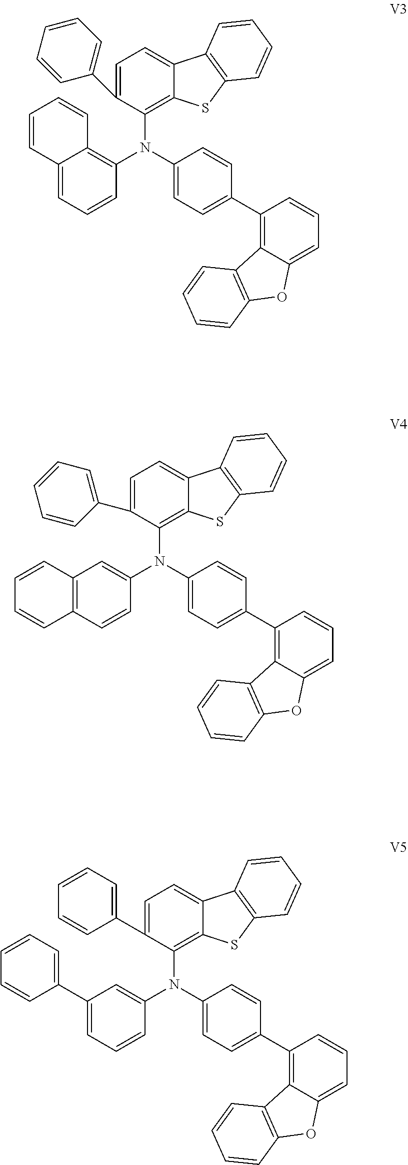 [SMILES: c1ccc(-c2ccc3c(sc4ccccc43)c2N(c2ccc(-c3cccc4oc5ccccc5c34)cc2)c2cccc3ccccc23)cc1 ; c1ccc(-c2ccc3c(sc4ccccc43)c2N(c2ccc(-c3cccc4oc5ccccc5c34)cc2)c2ccc3ccccc3c2)cc1 ; c1ccc(-c2cccc(N(c3ccc(-c4cccc5oc6ccccc6c45)cc3)c3c(-c4ccccc4)ccc4c3sc3ccccc34)c2)cc1] 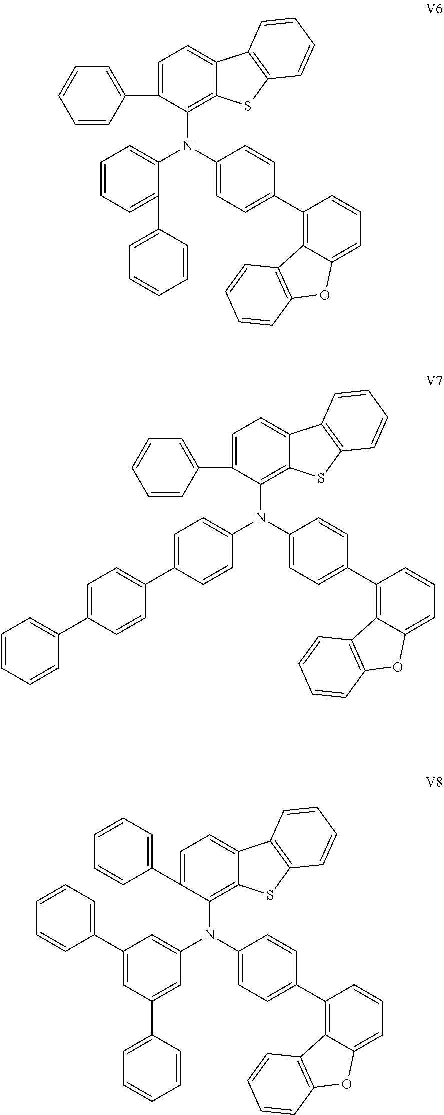 [SMILES: c1ccc(-c2ccccc2N(c2ccc(-c3cccc4oc5ccccc5c34)cc2)c2c(-c3ccccc3)ccc3c2sc2ccccc23)cc1 ; c1ccc(-c2ccc(-c3ccc(N(c4ccc(-c5cccc6oc7ccccc7c56)cc4)c4c(-c5ccccc5)ccc5c4sc4ccccc45)cc3)cc2)cc1 ; c1ccc(-c2cc(-c3ccccc3)cc(N(c3ccc(-c4cccc5oc6ccccc6c45)cc3)c3c(-c4ccccc4)ccc4c3sc3ccccc34)c2)cc1] 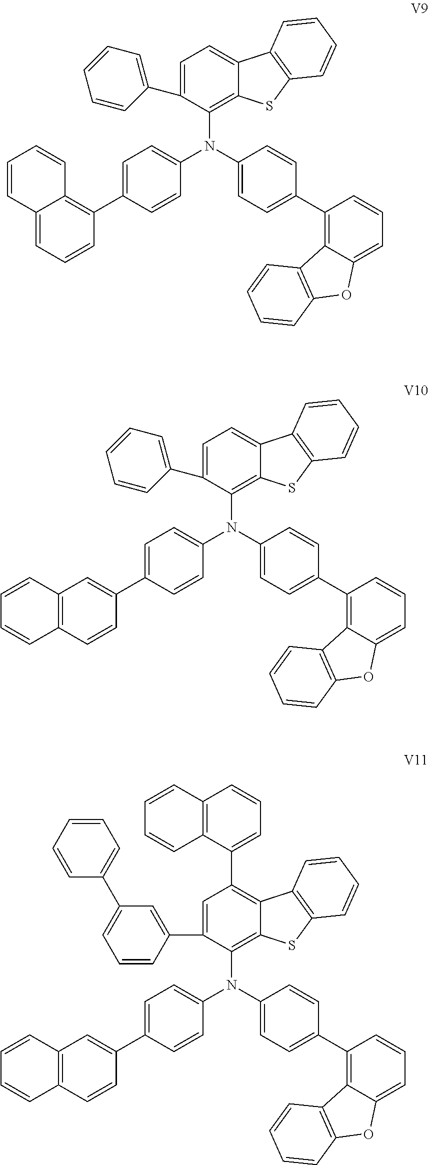 [SMILES: c1ccc(-c2ccc3c(sc4ccccc43)c2N(c2ccc(-c3cccc4ccccc34)cc2)c2ccc(-c3cccc4oc5ccccc5c34)cc2)cc1 ; c1ccc(-c2ccc3c(sc4ccccc43)c2N(c2ccc(-c3ccc4ccccc4c3)cc2)c2ccc(-c3cccc4oc5ccccc5c34)cc2)cc1 ; c1ccc(-c2cccc(-c3cc(-c4cccc5ccccc45)c4c(sc5ccccc54)c3N(c3ccc(-c4ccc5ccccc5c4)cc3)c3ccc(-c4cccc5oc6ccccc6c45)cc3)c2)cc1] 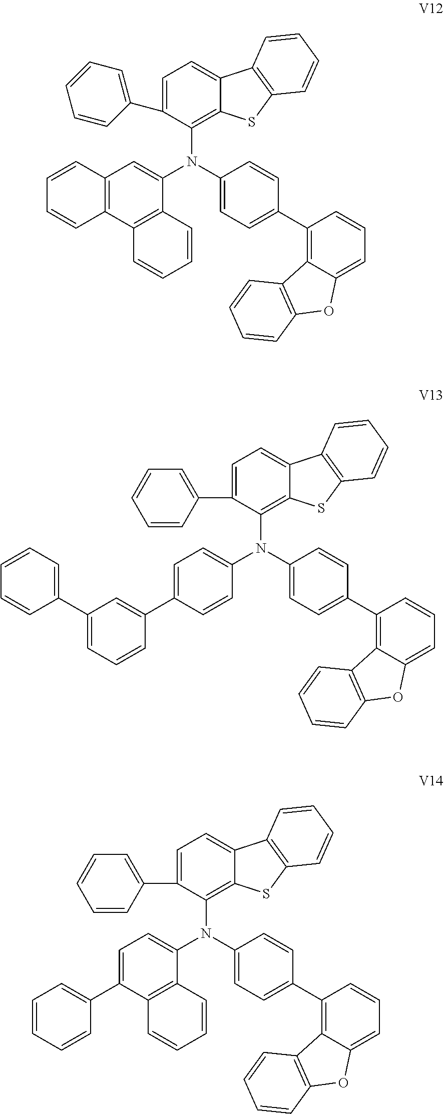 [SMILES: c1ccc(-c2ccc3c(sc4ccccc43)c2N(c2ccc(-c3cccc4oc5ccccc5c34)cc2)c2cc3ccccc3c3ccccc23)cc1 ; c1ccc(-c2cccc(-c3ccc(N(c4ccc(-c5cccc6oc7ccccc7c56)cc4)c4c(-c5ccccc5)ccc5c4sc4ccccc45)cc3)c2)cc1 ; c1ccc(-c2ccc3c(sc4ccccc43)c2N(c2ccc(-c3cccc4oc5ccccc5c34)cc2)c2ccc(-c3ccccc3)c3ccccc23)cc1] 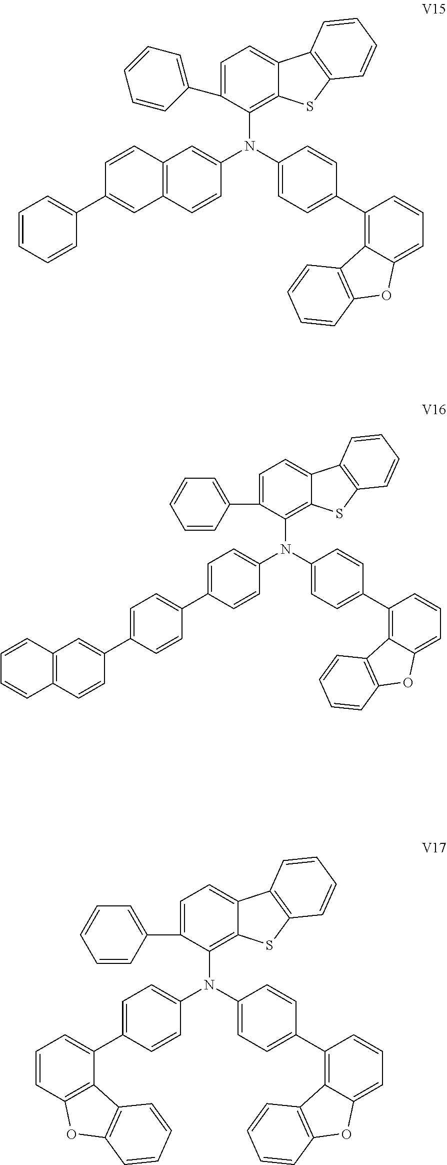 [SMILES: c1ccc(-c2ccc3cc(N(c4ccc(-c5cccc6oc7ccccc7c56)cc4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc(-c3cccc4oc5ccccc5c34)cc2)cc1 ; c1ccc(-c2ccc3c(sc4ccccc43)c2N(c2ccc(-c3cccc4oc5ccccc5c34)cc2)c2ccc(-c3cccc4oc5ccccc5c34)cc2)cc1] 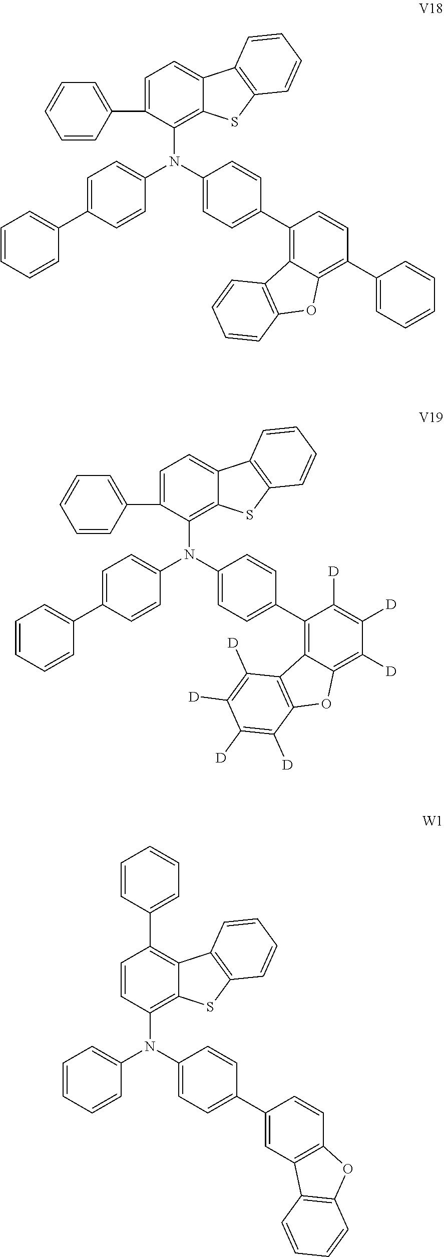 [SMILES: c1ccc(-c2ccc(N(c3ccc(-c4ccc(-c5ccccc5)c5oc6ccccc6c45)cc3)c3c(-c4ccccc4)ccc4c3sc3ccccc34)cc2)cc1 ; [2H]c1c([2H])c([2H])c2c(oc3c([2H])c([2H])c([2H])c(-c4ccc(N(c5ccc(-c6ccccc6)cc5)c5c(-c6ccccc6)ccc6c5sc5ccccc56)cc4)c32)c1[2H] ; c1ccc(-c2ccc(N(c3ccccc3)c3ccc(-c4ccc5oc6ccccc6c5c4)cc3)c3sc4ccccc4c23)cc1] 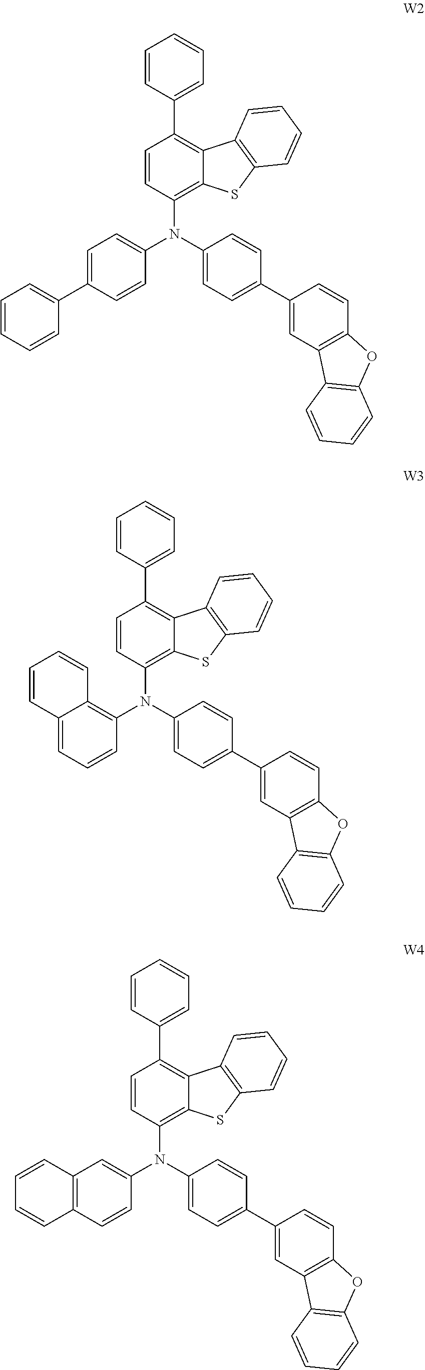 [SMILES: c1ccc(-c2ccc(N(c3ccc(-c4ccc5oc6ccccc6c5c4)cc3)c3ccc(-c4ccccc4)c4c3sc3ccccc34)cc2)cc1 ; c1ccc(-c2ccc(N(c3ccc(-c4ccc5oc6ccccc6c5c4)cc3)c3cccc4ccccc34)c3sc4ccccc4c23)cc1 ; c1ccc(-c2ccc(N(c3ccc(-c4ccc5oc6ccccc6c5c4)cc3)c3ccc4ccccc4c3)c3sc4ccccc4c23)cc1] 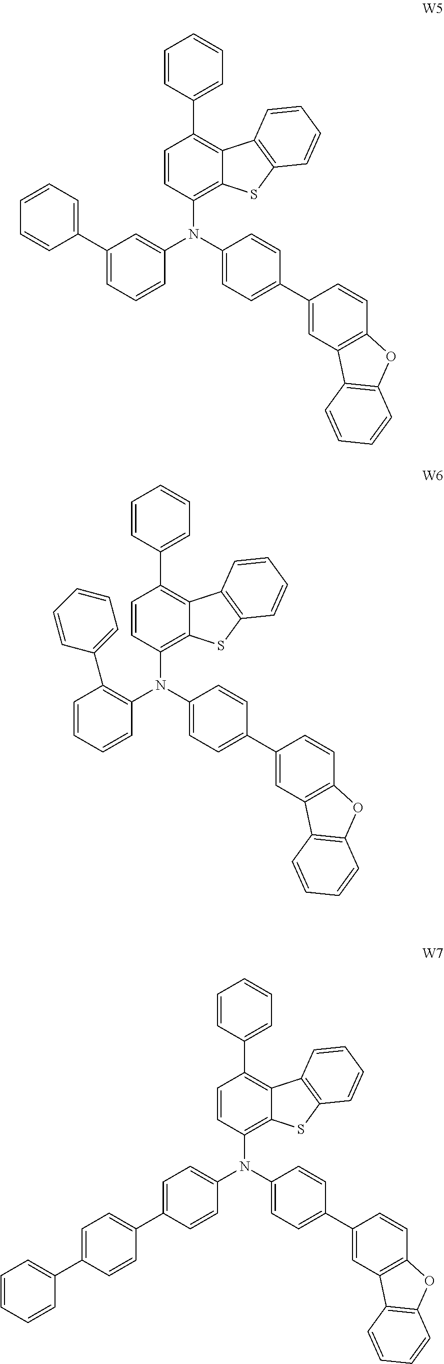 [SMILES: c1ccc(-c2cccc(N(c3ccc(-c4ccc5oc6ccccc6c5c4)cc3)c3ccc(-c4ccccc4)c4c3sc3ccccc34)c2)cc1 ; c1ccc(-c2ccccc2N(c2ccc(-c3ccc4oc5ccccc5c4c3)cc2)c2ccc(-c3ccccc3)c3c2sc2ccccc23)cc1 ; c1ccc(-c2ccc(-c3ccc(N(c4ccc(-c5ccc6oc7ccccc7c6c5)cc4)c4ccc(-c5ccccc5)c5c4sc4ccccc45)cc3)cc2)cc1] 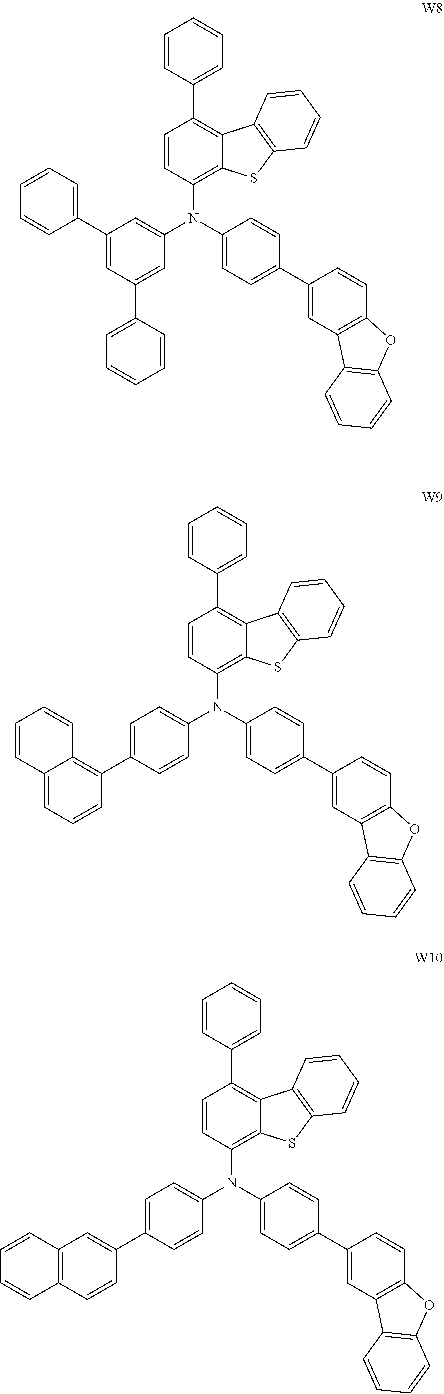 [SMILES: c1ccc(-c2cc(-c3ccccc3)cc(N(c3ccc(-c4ccc5oc6ccccc6c5c4)cc3)c3ccc(-c4ccccc4)c4c3sc3ccccc34)c2)cc1 ; c1ccc(-c2ccc(N(c3ccc(-c4ccc5oc6ccccc6c5c4)cc3)c3ccc(-c4cccc5ccccc45)cc3)c3sc4ccccc4c23)cc1 ; c1ccc(-c2ccc(N(c3ccc(-c4ccc5ccccc5c4)cc3)c3ccc(-c4ccc5oc6ccccc6c5c4)cc3)c3sc4ccccc4c23)cc1] 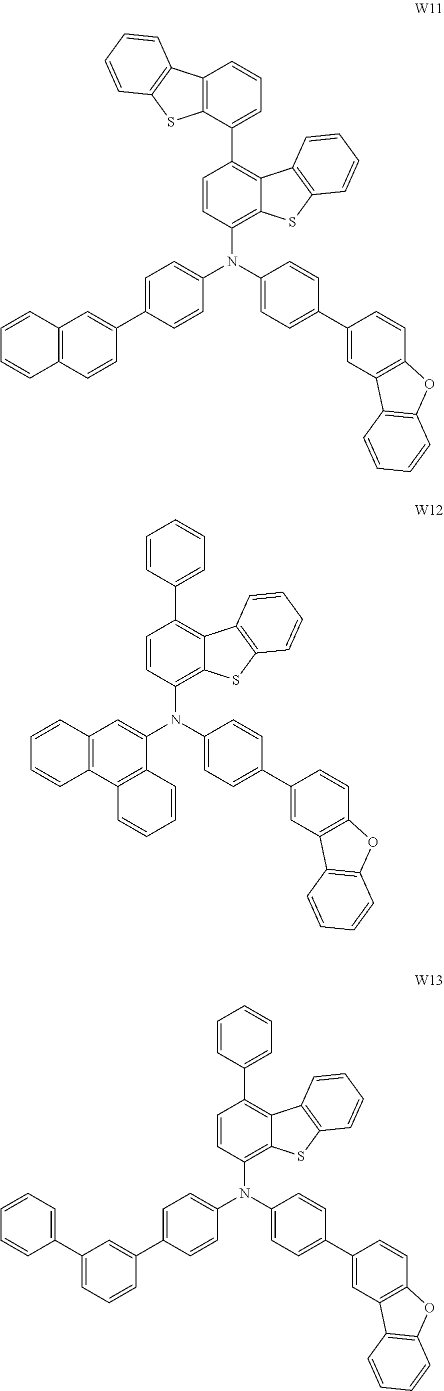 [SMILES: c1ccc2cc(-c3ccc(N(c4ccc(-c5ccc6oc7ccccc7c6c5)cc4)c4ccc(-c5cccc6c5sc5ccccc56)c5c4sc4ccccc45)cc3)ccc2c1 ; c1ccc(-c2ccc(N(c3ccc(-c4ccc5oc6ccccc6c5c4)cc3)c3cc4ccccc4c4ccccc34)c3sc4ccccc4c23)cc1 ; c1ccc(-c2cccc(-c3ccc(N(c4ccc(-c5ccc6oc7ccccc7c6c5)cc4)c4ccc(-c5ccccc5)c5c4sc4ccccc45)cc3)c2)cc1] 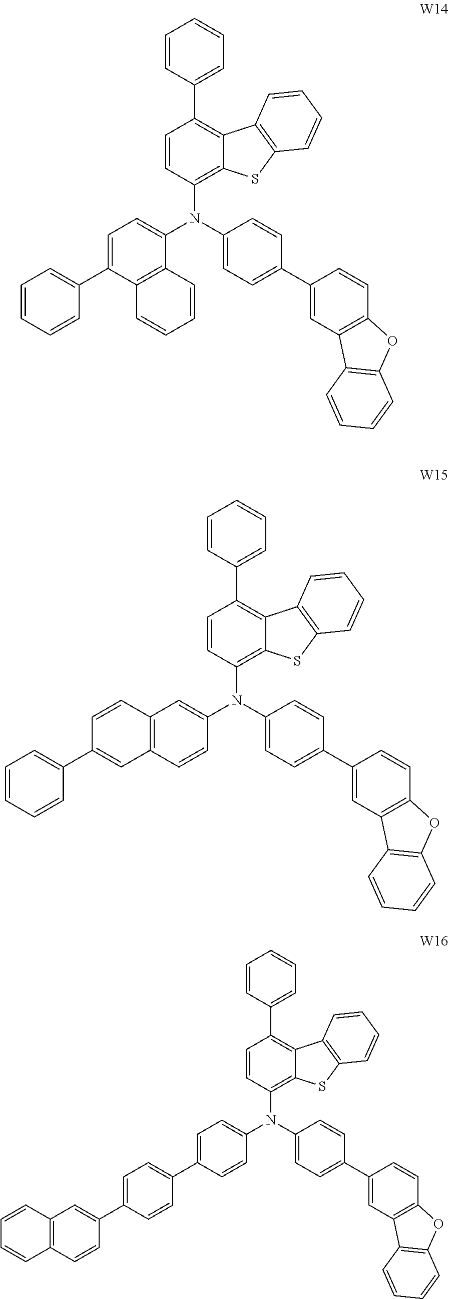 [SMILES: c1ccc(-c2ccc(N(c3ccc(-c4ccc5oc6ccccc6c5c4)cc3)c3ccc(-c4ccccc4)c4c3sc3ccccc34)c3ccccc23)cc1 ; c1ccc(-c2ccc3cc(N(c4ccc(-c5ccc6oc7ccccc7c6c5)cc4)c4ccc(-c5ccccc5)c5c4sc4ccccc45)ccc3c2)cc1 ; c1ccc(-c2ccc(N(c3ccc(-c4ccc(-c5ccc6ccccc6c5)cc4)cc3)c3ccc(-c4ccc5oc6ccccc6c5c4)cc3)c3sc4ccccc4c23)cc1] 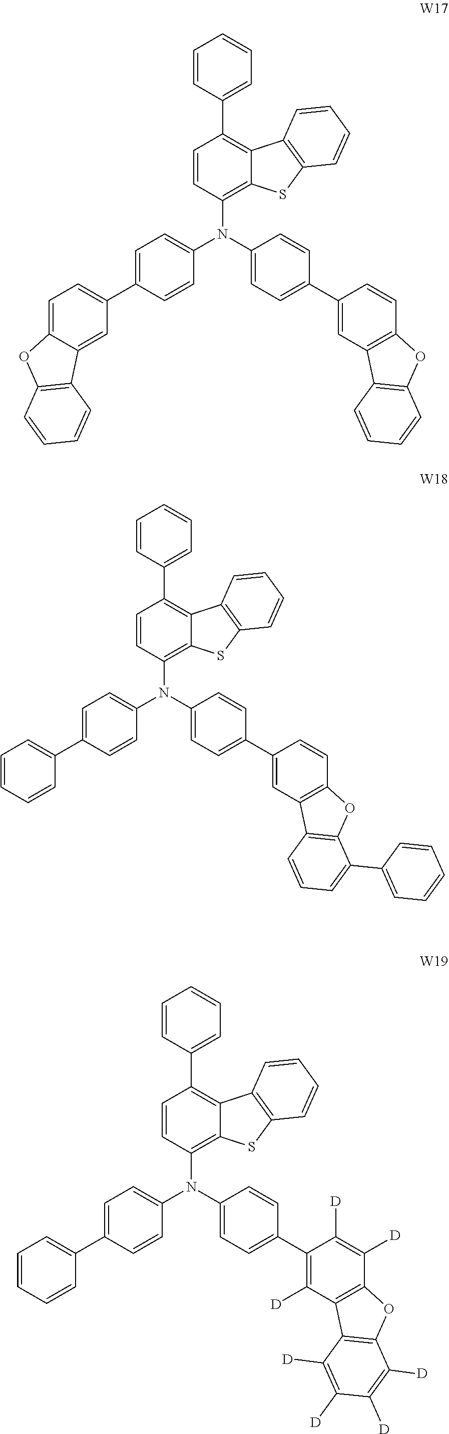 [SMILES: c1ccc(-c2ccc(N(c3ccc(-c4ccc5oc6ccccc6c5c4)cc3)c3ccc(-c4ccc5oc6ccccc6c5c4)cc3)c3sc4ccccc4c23)cc1 ; c1ccc(-c2ccc(N(c3ccc(-c4ccc5oc6c(-c7ccccc7)cccc6c5c4)cc3)c3ccc(-c4ccccc4)c4c3sc3ccccc34)cc2)cc1 ; [2H]c1c([2H])c([2H])c2c(oc3c([2H])c([2H])c(-c4ccc(N(c5ccc(-c6ccccc6)cc5)c5ccc(-c6ccccc6)c6c5sc5ccccc56)cc4)c([2H])c32)c1[2H]] 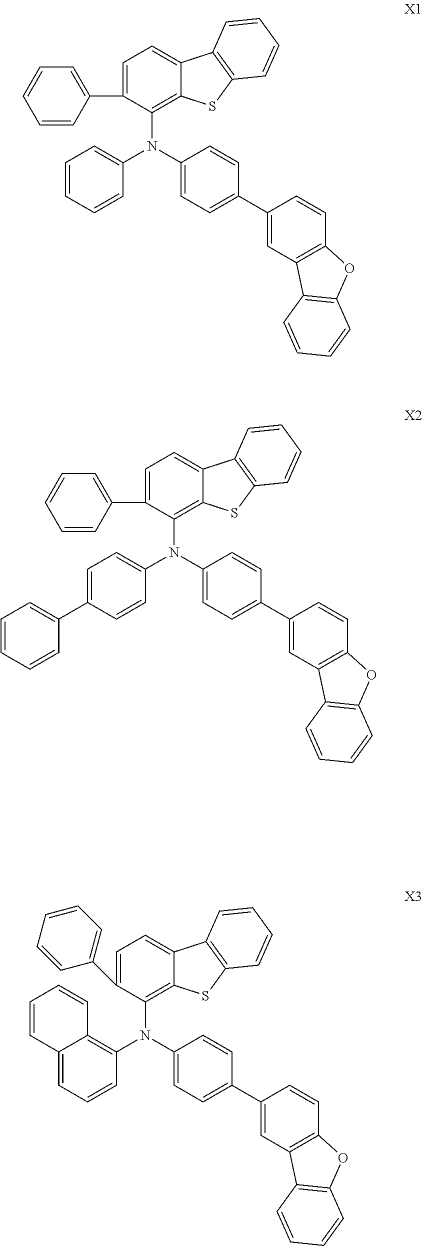 [SMILES: c1ccc(-c2ccc3c(sc4ccccc43)c2N(c2ccccc2)c2ccc(-c3ccc4oc5ccccc5c4c3)cc2)cc1 ; c1ccc(-c2ccc(N(c3ccc(-c4ccc5oc6ccccc6c5c4)cc3)c3c(-c4ccccc4)ccc4c3sc3ccccc34)cc2)cc1 ; c1ccc(-c2ccc3c(sc4ccccc43)c2N(c2ccc(-c3ccc4oc5ccccc5c4c3)cc2)c2cccc3ccccc23)cc1] 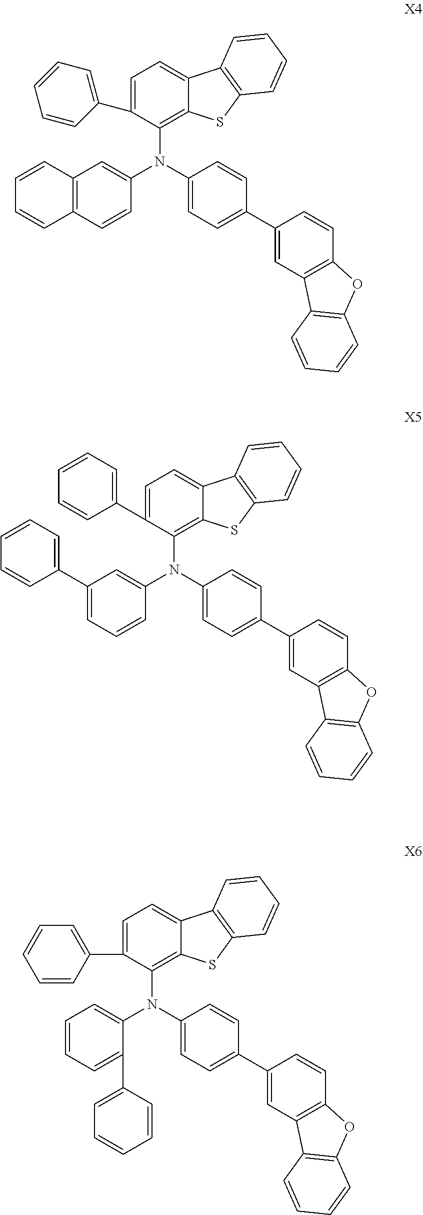 [SMILES: c1ccc(-c2ccc3c(sc4ccccc43)c2N(c2ccc(-c3ccc4oc5ccccc5c4c3)cc2)c2ccc3ccccc3c2)cc1 ; c1ccc(-c2cccc(N(c3ccc(-c4ccc5oc6ccccc6c5c4)cc3)c3c(-c4ccccc4)ccc4c3sc3ccccc34)c2)cc1 ; c1ccc(-c2ccccc2N(c2ccc(-c3ccc4oc5ccccc5c4c3)cc2)c2c(-c3ccccc3)ccc3c2sc2ccccc23)cc1] 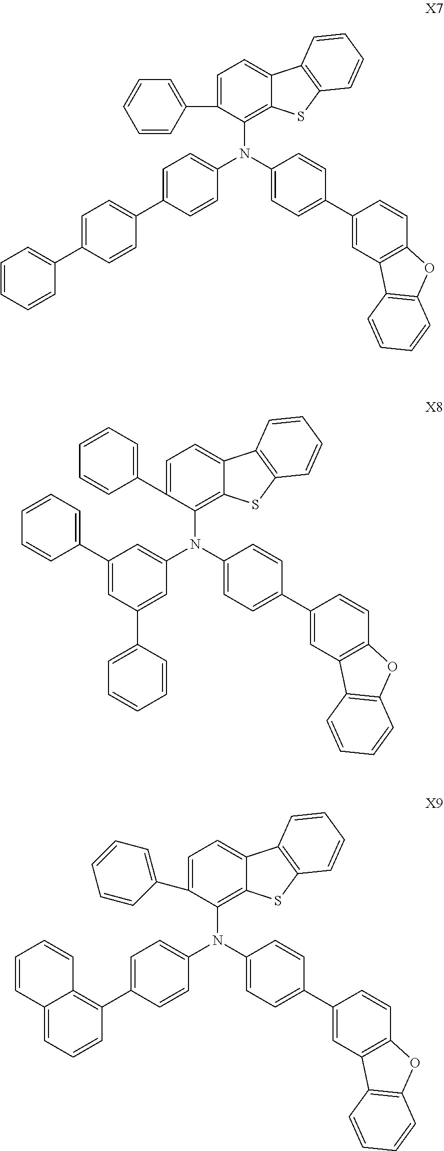 [SMILES: c1ccc(-c2ccc(-c3ccc(N(c4ccc(-c5ccc6oc7ccccc7c6c5)cc4)c4c(-c5ccccc5)ccc5c4sc4ccccc45)cc3)cc2)cc1 ; c1ccc(-c2cc(-c3ccccc3)cc(N(c3ccc(-c4ccc5oc6ccccc6c5c4)cc3)c3c(-c4ccccc4)ccc4c3sc3ccccc34)c2)cc1 ; c1ccc(-c2ccc3c(sc4ccccc43)c2N(c2ccc(-c3ccc4oc5ccccc5c4c3)cc2)c2ccc(-c3cccc4ccccc34)cc2)cc1] 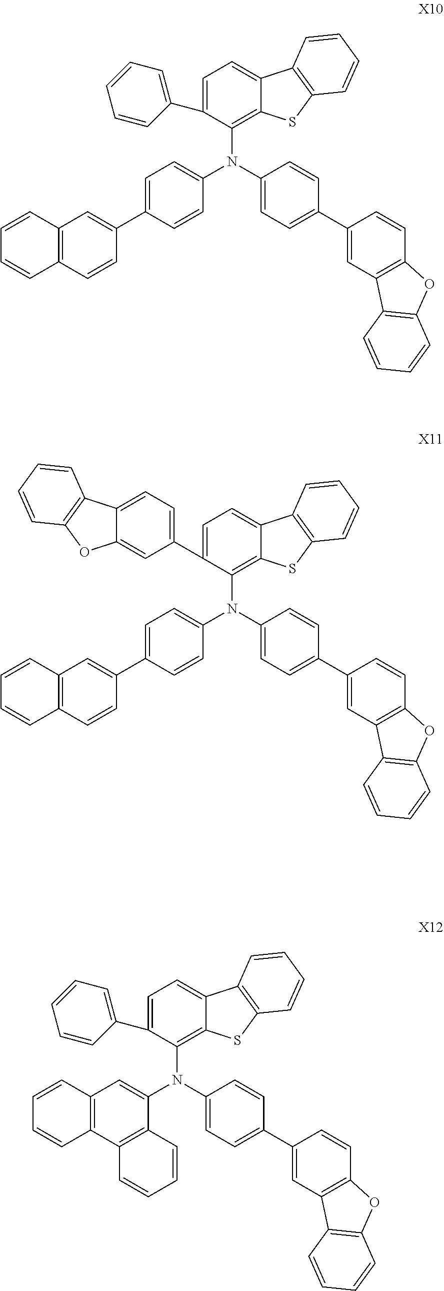 [SMILES: c1ccc(-c2ccc3c(sc4ccccc43)c2N(c2ccc(-c3ccc4ccccc4c3)cc2)c2ccc(-c3ccc4oc5ccccc5c4c3)cc2)cc1 ; c1ccc2cc(-c3ccc(N(c4ccc(-c5ccc6oc7ccccc7c6c5)cc4)c4c(-c5ccc6c(c5)oc5ccccc56)ccc5c4sc4ccccc45)cc3)ccc2c1 ; c1ccc(-c2ccc3c(sc4ccccc43)c2N(c2ccc(-c3ccc4oc5ccccc5c4c3)cc2)c2cc3ccccc3c3ccccc23)cc1] 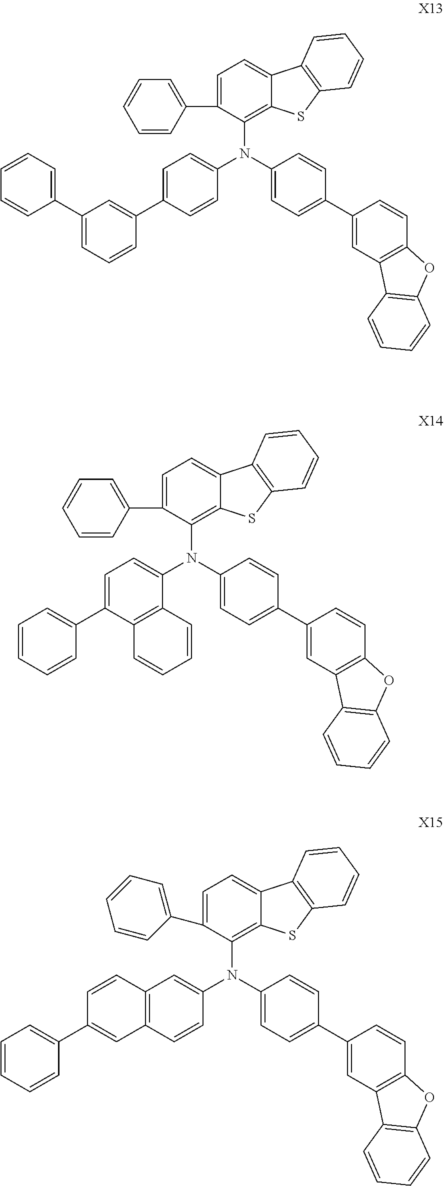 [SMILES: c1ccc(-c2cccc(-c3ccc(N(c4ccc(-c5ccc6oc7ccccc7c6c5)cc4)c4c(-c5ccccc5)ccc5c4sc4ccccc45)cc3)c2)cc1 ; c1ccc(-c2ccc3c(sc4ccccc43)c2N(c2ccc(-c3ccc4oc5ccccc5c4c3)cc2)c2ccc(-c3ccccc3)c3ccccc23)cc1 ; c1ccc(-c2ccc3cc(N(c4ccc(-c5ccc6oc7ccccc7c6c5)cc4)c4c(-c5ccccc5)ccc5c4sc4ccccc45)ccc3c2)cc1] 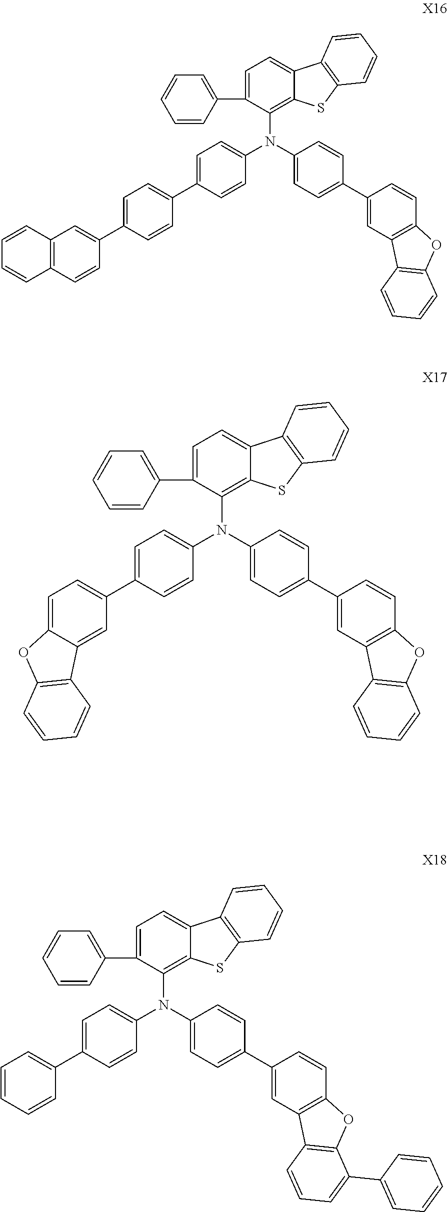 [SMILES: c1ccc(-c2ccc3c(sc4ccccc43)c2N(c2ccc(-c3ccc(-c4ccc5ccccc5c4)cc3)cc2)c2ccc(-c3ccc4oc5ccccc5c4c3)cc2)cc1 ; c1ccc(-c2ccc3c(sc4ccccc43)c2N(c2ccc(-c3ccc4oc5ccccc5c4c3)cc2)c2ccc(-c3ccc4oc5ccccc5c4c3)cc2)cc1 ; c1ccc(-c2ccc(N(c3ccc(-c4ccc5oc6c(-c7ccccc7)cccc6c5c4)cc3)c3c(-c4ccccc4)ccc4c3sc3ccccc34)cc2)cc1] 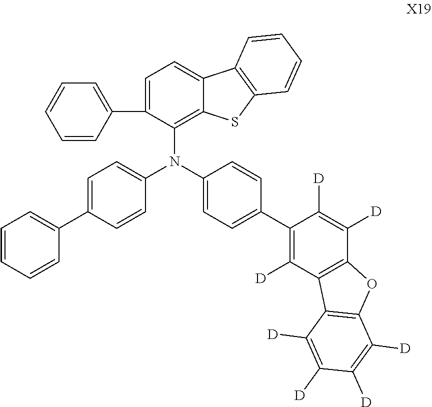 [SMILES: [2H]c1c([2H])c([2H])c2c(oc3c([2H])c([2H])c(-c4ccc(N(c5ccc(-c6ccccc6)cc5)c5c(-c6ccccc6)ccc6c5sc5ccccc56)cc4)c([2H])c32)c1[2H]] 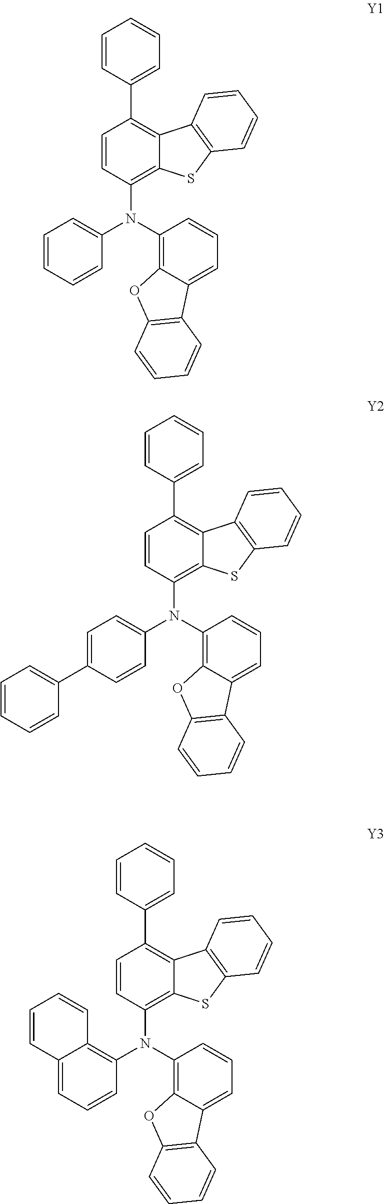 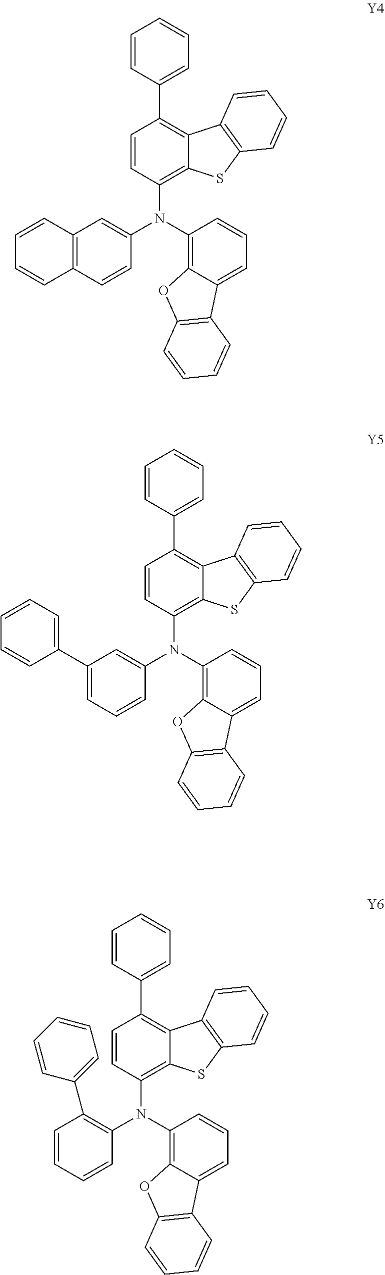 [SMILES: c1ccc(-c2ccc(N(c3ccc4ccccc4c3)c3cccc4c3oc3ccccc34)c3sc4ccccc4c23)cc1 ; c1ccc(-c2cccc(N(c3cccc4c3oc3ccccc34)c3ccc(-c4ccccc4)c4c3sc3ccccc34)c2)cc1 ; c1ccc(-c2ccccc2N(c2cccc3c2oc2ccccc23)c2ccc(-c3ccccc3)c3c2sc2ccccc23)cc1] 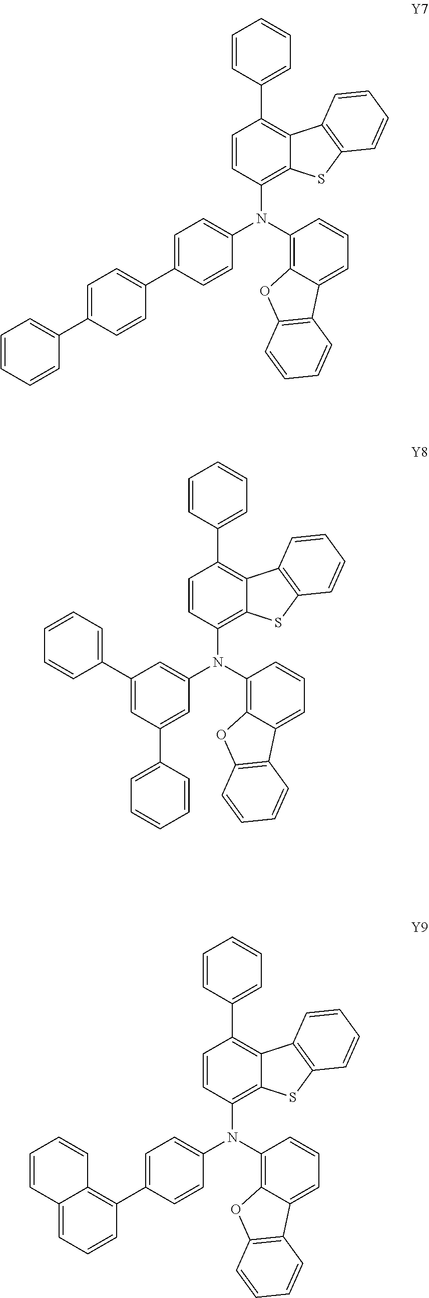 [SMILES: c1ccc(-c2ccc(-c3ccc(N(c4cccc5c4oc4ccccc45)c4ccc(-c5ccccc5)c5c4sc4ccccc45)cc3)cc2)cc1 ; c1ccc(-c2cc(-c3ccccc3)cc(N(c3cccc4c3oc3ccccc34)c3ccc(-c4ccccc4)c4c3sc3ccccc34)c2)cc1 ; c1ccc(-c2ccc(N(c3ccc(-c4cccc5ccccc45)cc3)c3cccc4c3oc3ccccc34)c3sc4ccccc4c23)cc1] 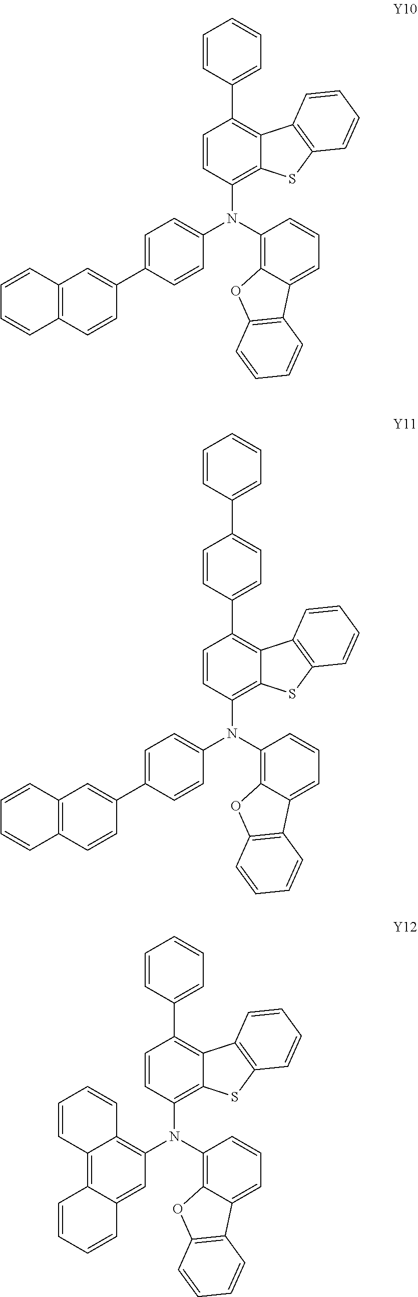 [SMILES: c1ccc(-c2ccc(N(c3ccc(-c4ccc5ccccc5c4)cc3)c3cccc4c3oc3ccccc34)c3sc4ccccc4c23)cc1 ; c1ccc(-c2ccc(-c3ccc(N(c4ccc(-c5ccc6ccccc6c5)cc4)c4cccc5c4oc4ccccc45)c4sc5ccccc5c34)cc2)cc1 ; c1ccc(-c2ccc(N(c3cc4ccccc4c4ccccc34)c3cccc4c3oc3ccccc34)c3sc4ccccc4c23)cc1] 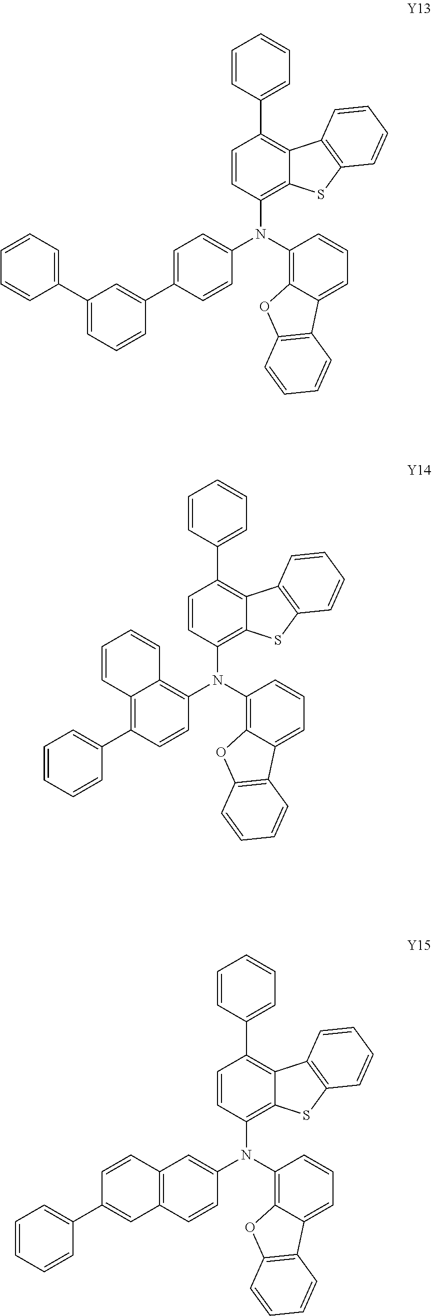 [SMILES: c1ccc(-c2cccc(-c3ccc(N(c4cccc5c4oc4ccccc45)c4ccc(-c5ccccc5)c5c4sc4ccccc45)cc3)c2)cc1 ; c1ccc(-c2ccc(N(c3cccc4c3oc3ccccc34)c3ccc(-c4ccccc4)c4c3sc3ccccc34)c3ccccc23)cc1 ; c1ccc(-c2ccc3cc(N(c4cccc5c4oc4ccccc45)c4ccc(-c5ccccc5)c5c4sc4ccccc45)ccc3c2)cc1] 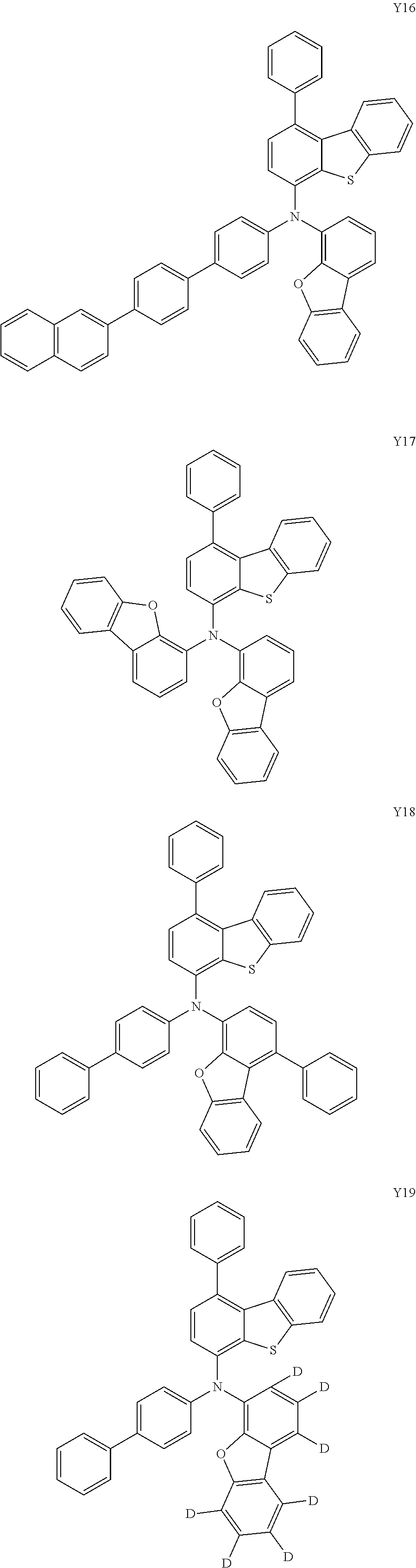 [SMILES: c1ccc(-c2ccc(N(c3ccc(-c4ccc(-c5ccc6ccccc6c5)cc4)cc3)c3cccc4c3oc3ccccc34)c3sc4ccccc4c23)cc1 ; c1ccc(-c2ccc(N(c3cccc4c3oc3ccccc34)c3cccc4c3oc3ccccc34)c3sc4ccccc4c23)cc1 ; c1ccc(-c2ccc(N(c3ccc(-c4ccccc4)c4c3oc3ccccc34)c3ccc(-c4ccccc4)c4c3sc3ccccc34)cc2)cc1 ; [2H]c1c([2H])c([2H])c2c(oc3c(N(c4ccc(-c5ccccc5)cc4)c4ccc(-c5ccccc5)c5c4sc4ccccc45)c([2H])c([2H])c([2H])c32)c1[2H]] 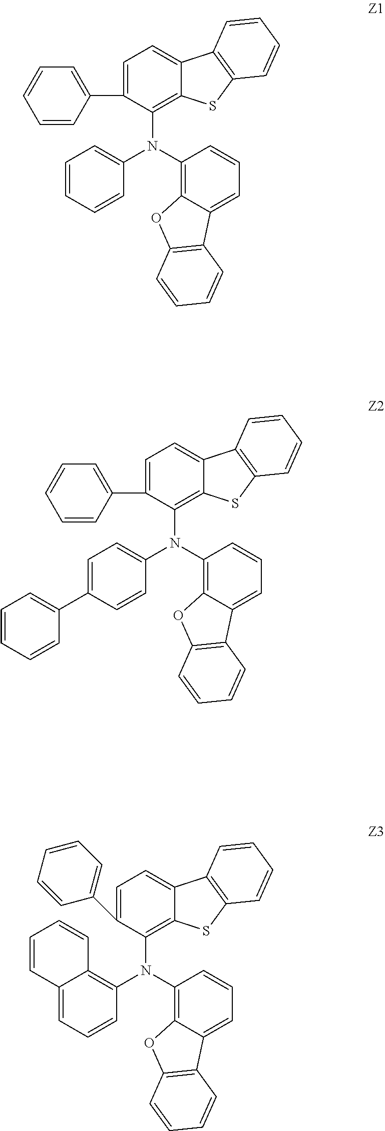 [SMILES: c1ccc(-c2ccc3c(sc4ccccc43)c2N(c2ccccc2)c2cccc3c2oc2ccccc23)cc1 ; c1ccc(-c2ccc(N(c3cccc4c3oc3ccccc34)c3c(-c4ccccc4)ccc4c3sc3ccccc34)cc2)cc1 ; c1ccc(-c2ccc3c(sc4ccccc43)c2N(c2cccc3ccccc23)c2cccc3c2oc2ccccc23)cc1] 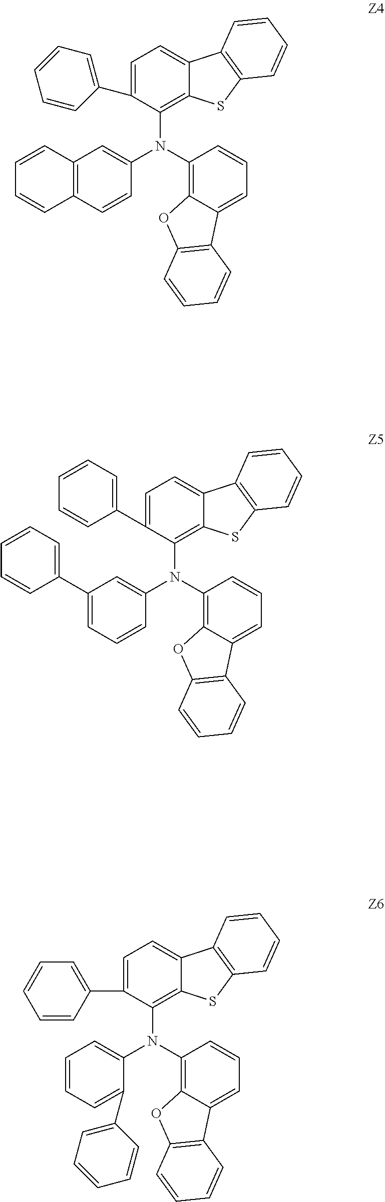 [SMILES: c1ccc(-c2ccc3c(sc4ccccc43)c2N(c2ccc3ccccc3c2)c2cccc3c2oc2ccccc23)cc1 ; c1ccc(-c2cccc(N(c3cccc4c3oc3ccccc34)c3c(-c4ccccc4)ccc4c3sc3ccccc34)c2)cc1 ; c1ccc(-c2ccccc2N(c2cccc3c2oc2ccccc23)c2c(-c3ccccc3)ccc3c2sc2ccccc23)cc1] 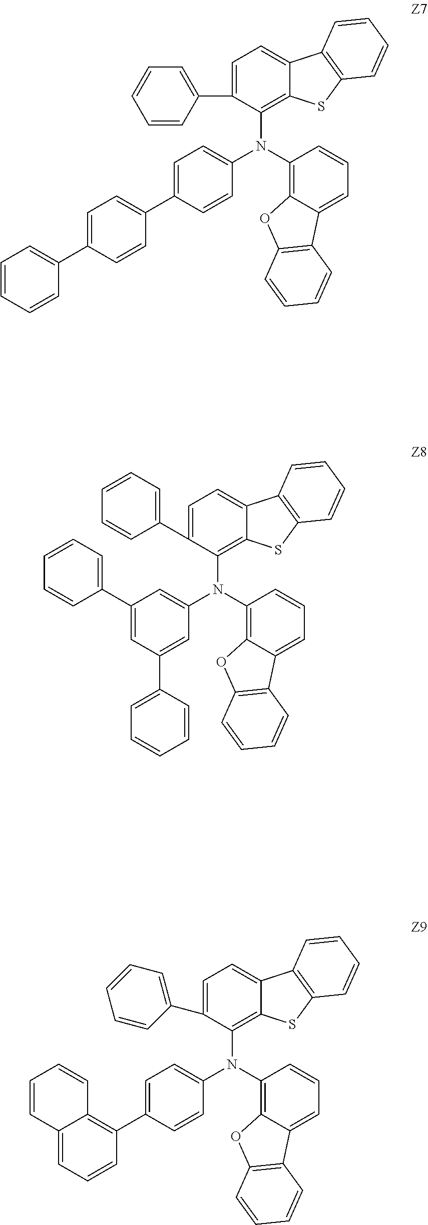 [SMILES: c1ccc(-c2ccc(-c3ccc(N(c4cccc5c4oc4ccccc45)c4c(-c5ccccc5)ccc5c4sc4ccccc45)cc3)cc2)cc1 ; c1ccc(-c2cc(-c3ccccc3)cc(N(c3cccc4c3oc3ccccc34)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2cccc3c2oc2ccccc23)cc1] 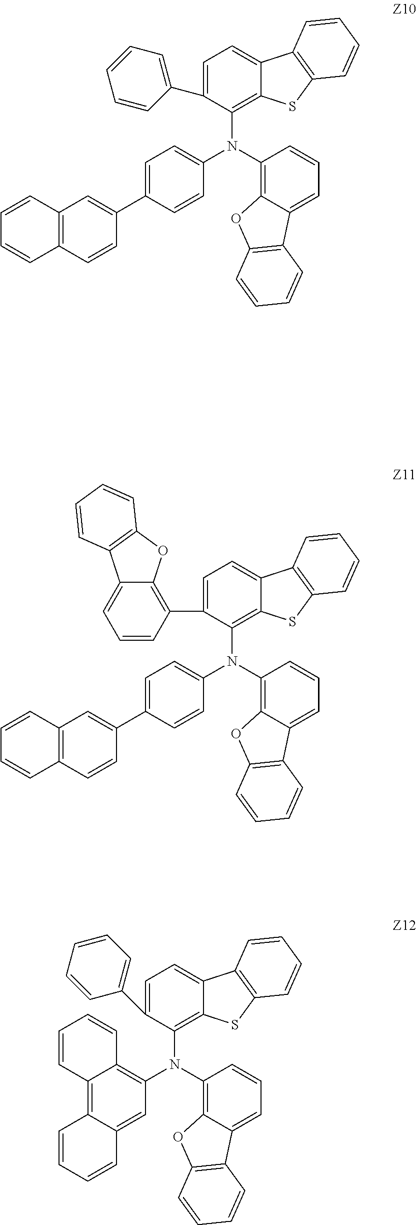 [SMILES: c1ccc(-c2ccc3c(sc4ccccc43)c2N(c2ccc(-c3ccc4ccccc4c3)cc2)c2cccc3c2oc2ccccc23)cc1 ; c1ccc2cc(-c3ccc(N(c4cccc5c4oc4ccccc45)c4c(-c5cccc6c5oc5ccccc56)ccc5c4sc4ccccc45)cc3)ccc2c1 ; c1ccc(-c2ccc3c(sc4ccccc43)c2N(c2cc3ccccc3c3ccccc23)c2cccc3c2oc2ccccc23)cc1] 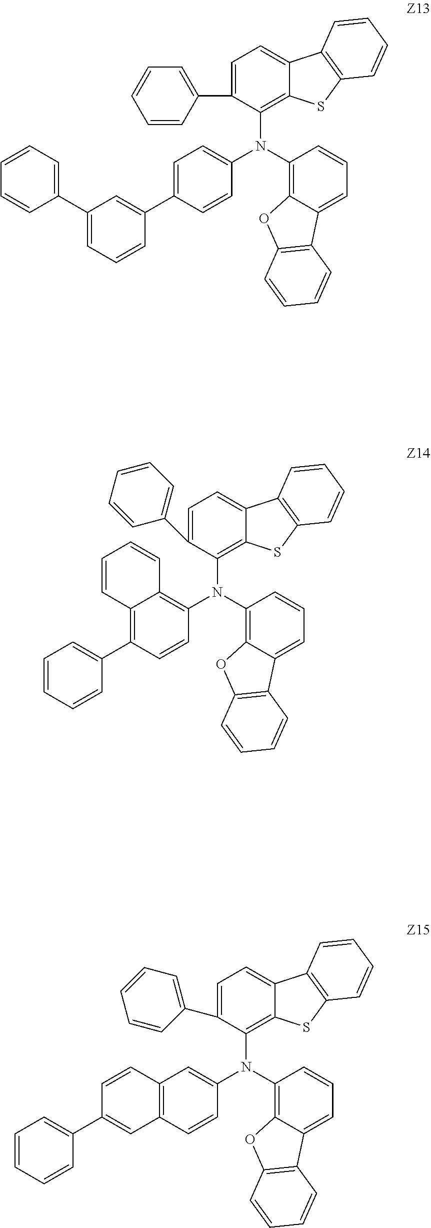 [SMILES: c1ccc(-c2cccc(-c3ccc(N(c4cccc5c4oc4ccccc45)c4c(-c5ccccc5)ccc5c4sc4ccccc45)cc3)c2)cc1 ; c1ccc(-c2ccc3c(sc4ccccc43)c2N(c2ccc(-c3ccccc3)c3ccccc23)c2cccc3c2oc2ccccc23)cc1 ; c1ccc(-c2ccc3cc(N(c4cccc5c4oc4ccccc45)c4c(-c5ccccc5)ccc5c4sc4ccccc45)ccc3c2)cc1] 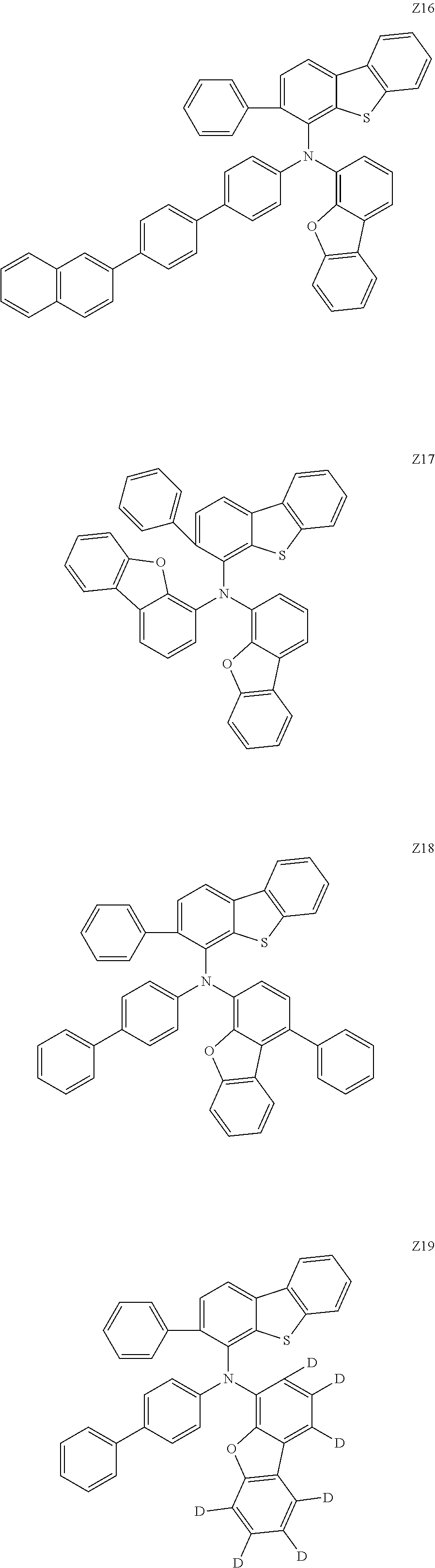 [SMILES: c1ccc(-c2ccc3c(sc4ccccc43)c2N(c2ccc(-c3ccc(-c4ccc5ccccc5c4)cc3)cc2)c2cccc3c2oc2ccccc23)cc1 ; c1ccc(-c2ccc3c(sc4ccccc43)c2N(c2cccc3c2oc2ccccc23)c2cccc3c2oc2ccccc23)cc1 ; c1ccc(-c2ccc(N(c3ccc(-c4ccccc4)c4c3oc3ccccc34)c3c(-c4ccccc4)ccc4c3sc3ccccc34)cc2)cc1 ; [2H]c1c([2H])c([2H])c2c(oc3c(N(c4ccc(-c5ccccc5)cc4)c4c(-c5ccccc5)ccc5c4sc4ccccc45)c([2H])c([2H])c([2H])c32)c1[2H]] 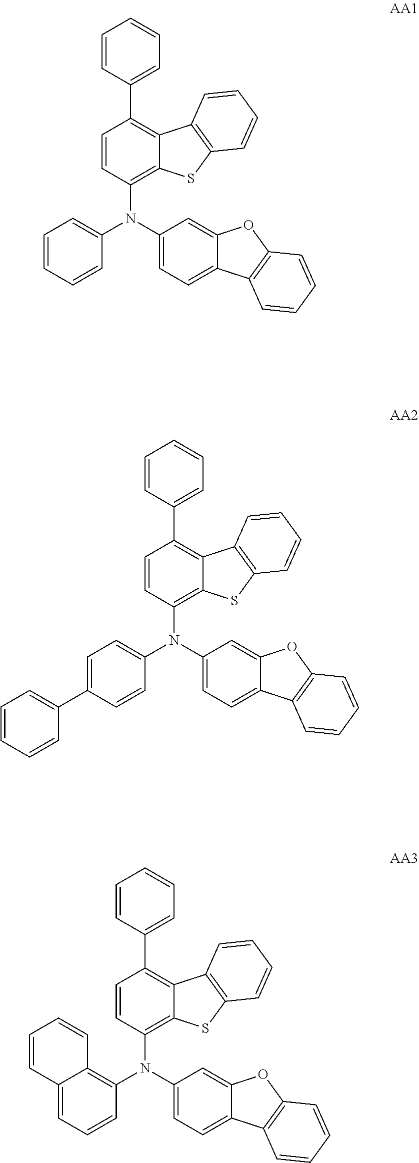 [SMILES: c1ccc(-c2ccc(N(c3ccccc3)c3ccc4c(c3)oc3ccccc34)c3sc4ccccc4c23)cc1 ; c1ccc(-c2ccc(N(c3ccc4c(c3)oc3ccccc34)c3ccc(-c4ccccc4)c4c3sc3ccccc34)cc2)cc1 ; c1ccc(-c2ccc(N(c3ccc4c(c3)oc3ccccc34)c3cccc4ccccc34)c3sc4ccccc4c23)cc1] 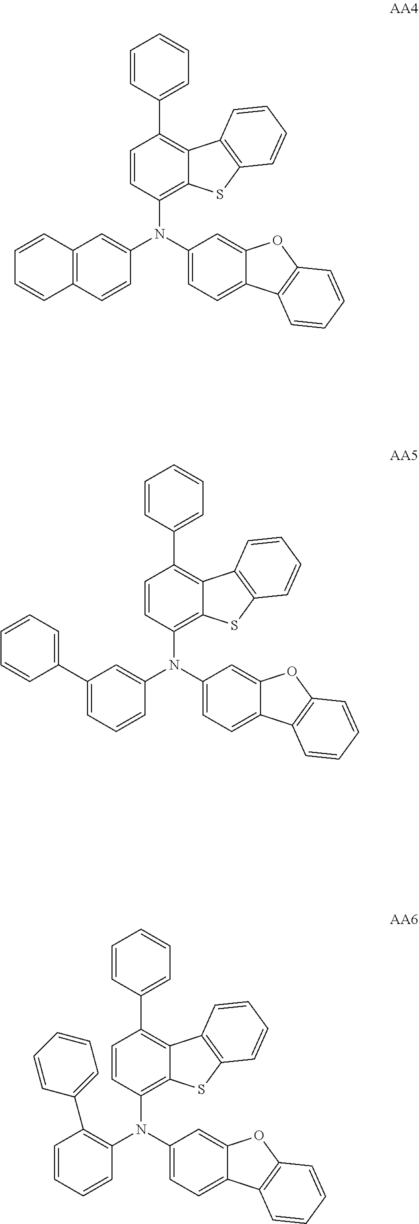 [SMILES: c1ccc(-c2ccc(N(c3ccc4ccccc4c3)c3ccc4c(c3)oc3ccccc34)c3sc4ccccc4c23)cc1 ; c1ccc(-c2cccc(N(c3ccc4c(c3)oc3ccccc34)c3ccc(-c4ccccc4)c4c3sc3ccccc34)c2)cc1 ; c1ccc(-c2ccccc2N(c2ccc3c(c2)oc2ccccc23)c2ccc(-c3ccccc3)c3c2sc2ccccc23)cc1] 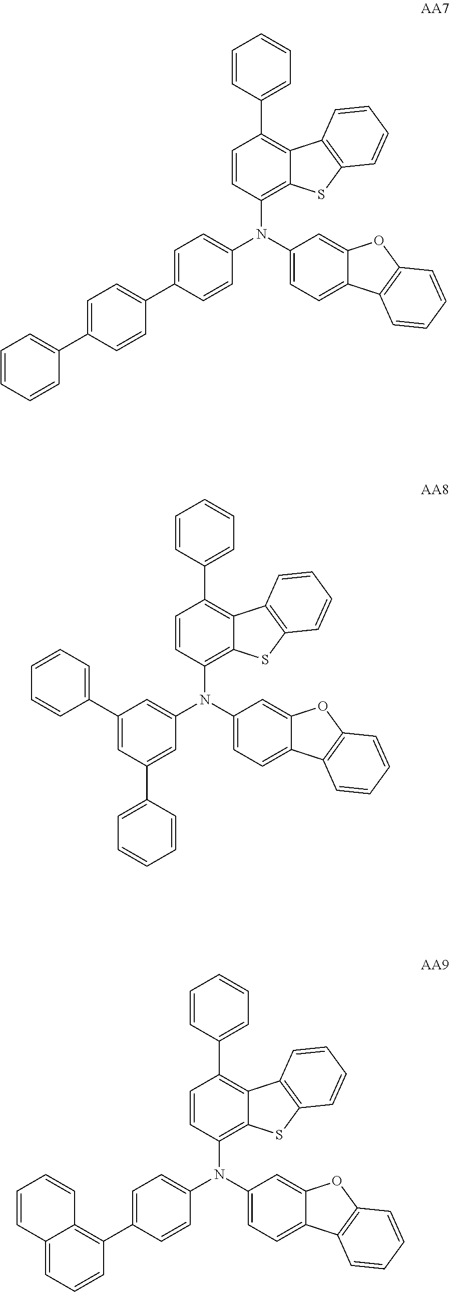 [SMILES: c1ccc(-c2ccc(-c3ccc(N(c4ccc5c(c4)oc4ccccc45)c4ccc(-c5ccccc5)c5c4sc4ccccc45)cc3)cc2)cc1 ; c1ccc(-c2cc(-c3ccccc3)cc(N(c3ccc4c(c3)oc3ccccc34)c3ccc(-c4ccccc4)c4c3sc3ccccc34)c2)cc1 ; c1ccc(-c2ccc(N(c3ccc(-c4cccc5ccccc45)cc3)c3ccc4c(c3)oc3ccccc34)c3sc4ccccc4c23)cc1] 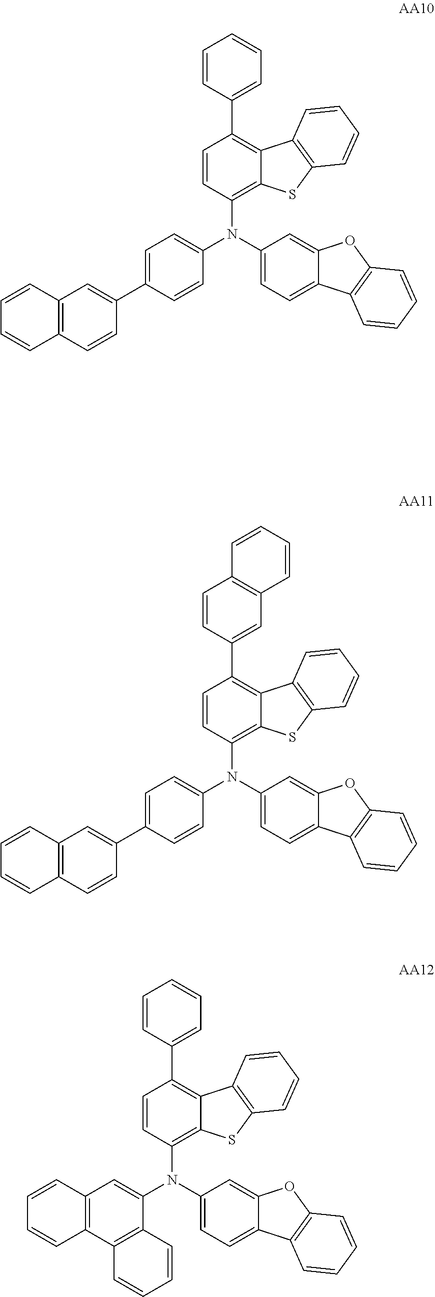 [SMILES: c1ccc(-c2ccc(N(c3ccc(-c4ccc5ccccc5c4)cc3)c3ccc4c(c3)oc3ccccc34)c3sc4ccccc4c23)cc1 ; c1ccc2cc(-c3ccc(N(c4ccc5c(c4)oc4ccccc45)c4ccc(-c5ccc6ccccc6c5)c5c4sc4ccccc45)cc3)ccc2c1 ; c1ccc(-c2ccc(N(c3ccc4c(c3)oc3ccccc34)c3cc4ccccc4c4ccccc34)c3sc4ccccc4c23)cc1] 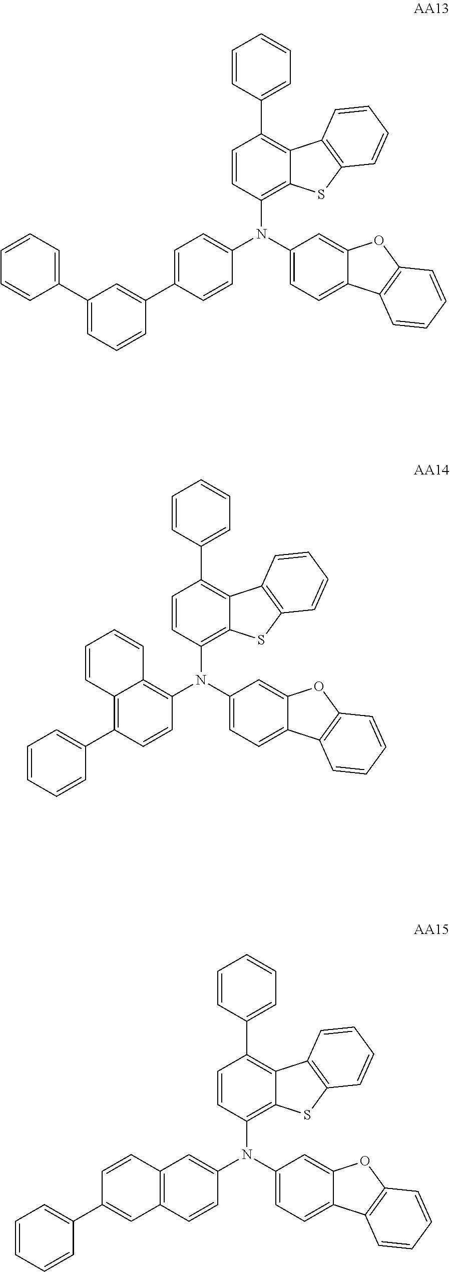 [SMILES: c1ccc(-c2cccc(-c3ccc(N(c4ccc5c(c4)oc4ccccc45)c4ccc(-c5ccccc5)c5c4sc4ccccc45)cc3)c2)cc1 ; c1ccc(-c2ccc(N(c3ccc4c(c3)oc3ccccc34)c3ccc(-c4ccccc4)c4c3sc3ccccc34)c3ccccc23)cc1 ; c1ccc(-c2ccc3cc(N(c4ccc5c(c4)oc4ccccc45)c4ccc(-c5ccccc5)c5c4sc4ccccc45)ccc3c2)cc1] 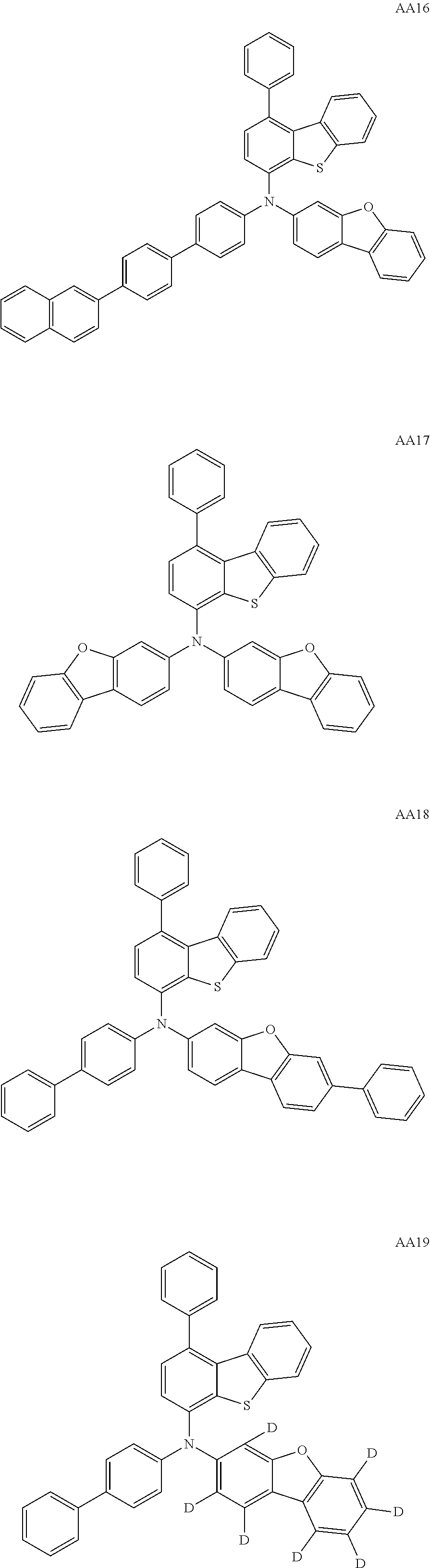 [SMILES: c1ccc(-c2ccc(N(c3ccc(-c4ccc(-c5ccc6ccccc6c5)cc4)cc3)c3ccc4c(c3)oc3ccccc34)c3sc4ccccc4c23)cc1 ; c1ccc(-c2ccc(N(c3ccc4c(c3)oc3ccccc34)c3ccc4c(c3)oc3ccccc34)c3sc4ccccc4c23)cc1 ; c1ccc(-c2ccc(N(c3ccc4c(c3)oc3cc(-c5ccccc5)ccc34)c3ccc(-c4ccccc4)c4c3sc3ccccc34)cc2)cc1 ; [2H]c1c([2H])c([2H])c2c(oc3c([2H])c(N(c4ccc(-c5ccccc5)cc4)c4ccc(-c5ccccc5)c5c4sc4ccccc45)c([2H])c([2H])c32)c1[2H]] 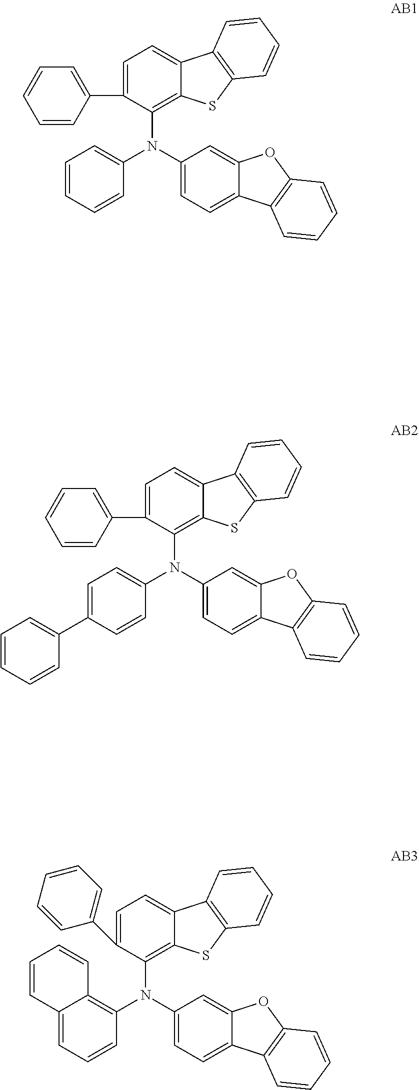 [SMILES: c1ccc(-c2ccc3c(sc4ccccc43)c2N(c2ccccc2)c2ccc3c(c2)oc2ccccc23)cc1 ; c1ccc(-c2ccc(N(c3ccc4c(c3)oc3ccccc34)c3c(-c4ccccc4)ccc4c3sc3ccccc34)cc2)cc1 ; c1ccc(-c2ccc3c(sc4ccccc43)c2N(c2ccc3c(c2)oc2ccccc23)c2cccc3ccccc23)cc1] 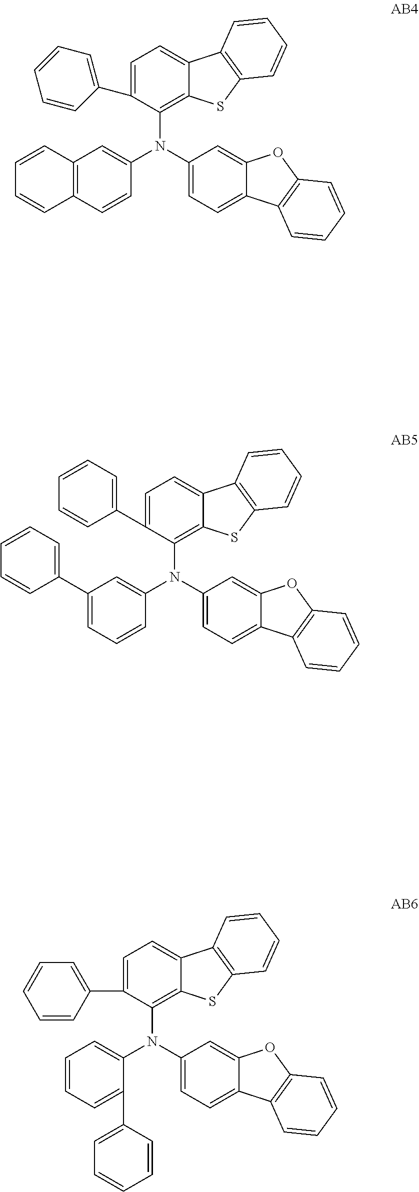 [SMILES: c1ccc(-c2ccc3c(sc4ccccc43)c2N(c2ccc3ccccc3c2)c2ccc3c(c2)oc2ccccc23)cc1 ; c1ccc(-c2cccc(N(c3ccc4c(c3)oc3ccccc34)c3c(-c4ccccc4)ccc4c3sc3ccccc34)c2)cc1 ; c1ccc(-c2ccccc2N(c2ccc3c(c2)oc2ccccc23)c2c(-c3ccccc3)ccc3c2sc2ccccc23)cc1] 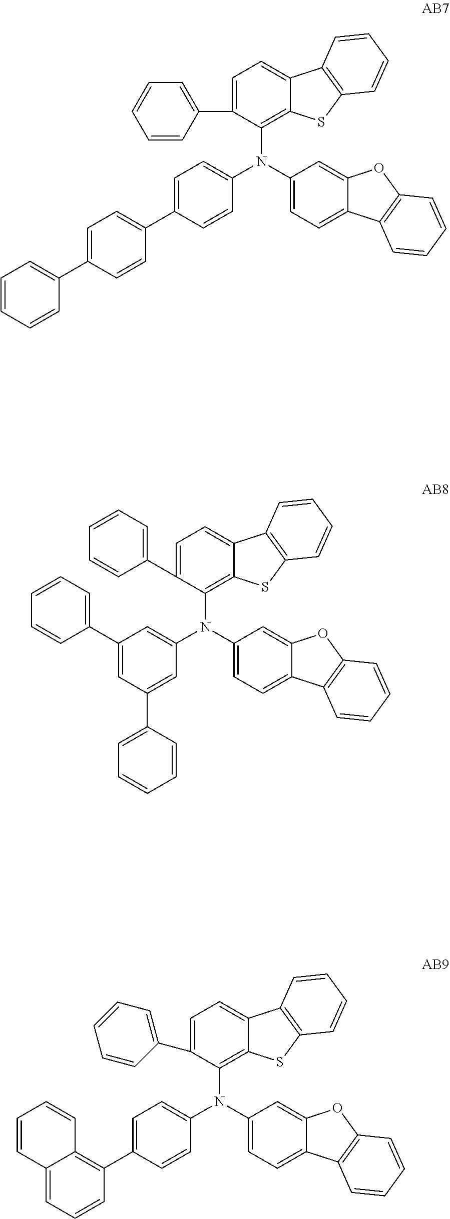 [SMILES: c1ccc(-c2ccc(-c3ccc(N(c4ccc5c(c4)oc4ccccc45)c4c(-c5ccccc5)ccc5c4sc4ccccc45)cc3)cc2)cc1 ; c1ccc(-c2cc(-c3ccccc3)cc(N(c3ccc4c(c3)oc3ccccc34)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2ccc3c(c2)oc2ccccc23)cc1] 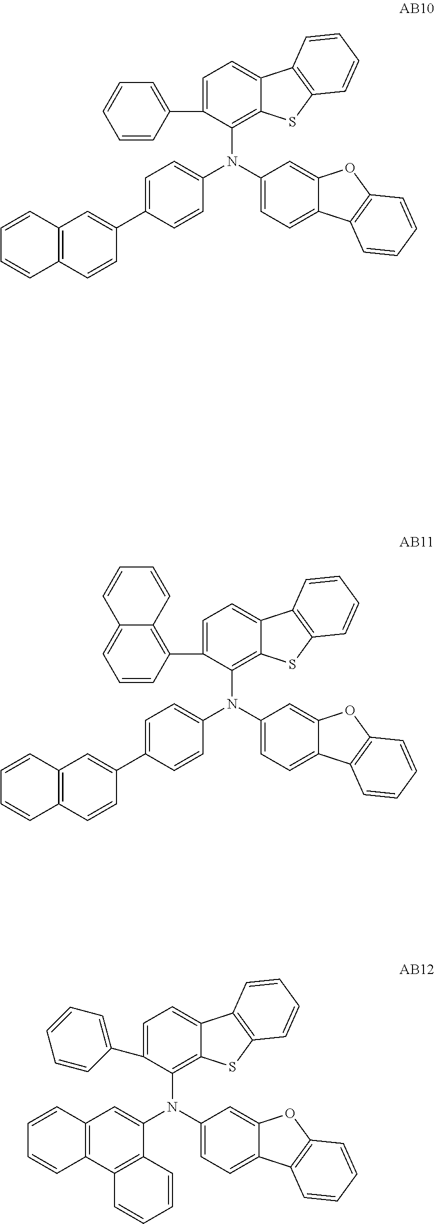 [SMILES: c1ccc(-c2ccc3c(sc4ccccc43)c2N(c2ccc(-c3ccc4ccccc4c3)cc2)c2ccc3c(c2)oc2ccccc23)cc1 ; c1ccc2cc(-c3ccc(N(c4ccc5c(c4)oc4ccccc45)c4c(-c5cccc6ccccc56)ccc5c4sc4ccccc45)cc3)ccc2c1 ; c1ccc(-c2ccc3c(sc4ccccc43)c2N(c2ccc3c(c2)oc2ccccc23)c2cc3ccccc3c3ccccc23)cc1] 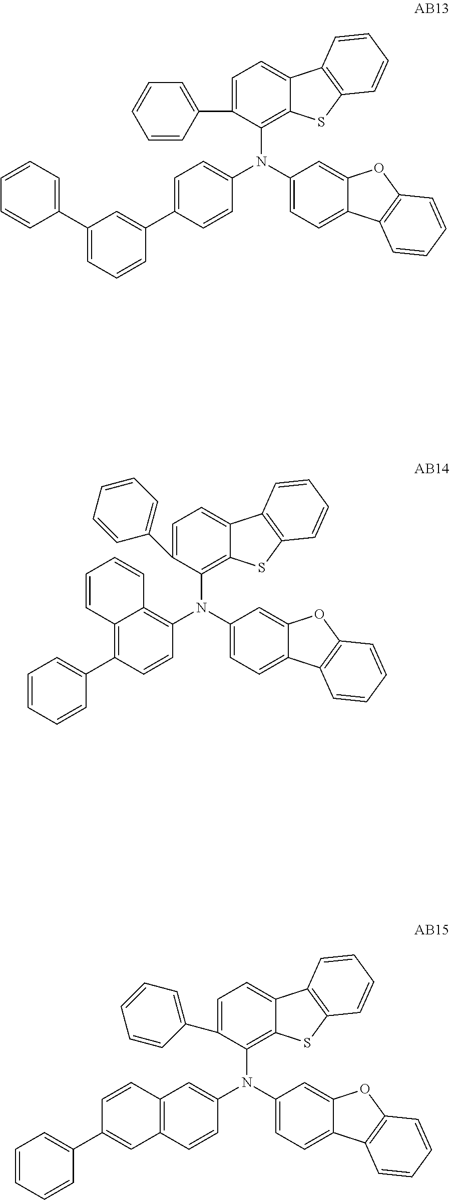 [SMILES: c1ccc(-c2cccc(-c3ccc(N(c4ccc5c(c4)oc4ccccc45)c4c(-c5ccccc5)ccc5c4sc4ccccc45)cc3)c2)cc1 ; c1ccc(-c2ccc3c(sc4ccccc43)c2N(c2ccc3c(c2)oc2ccccc23)c2ccc(-c3ccccc3)c3ccccc23)cc1 ; c1ccc(-c2ccc3cc(N(c4ccc5c(c4)oc4ccccc45)c4c(-c5ccccc5)ccc5c4sc4ccccc45)ccc3c2)cc1] 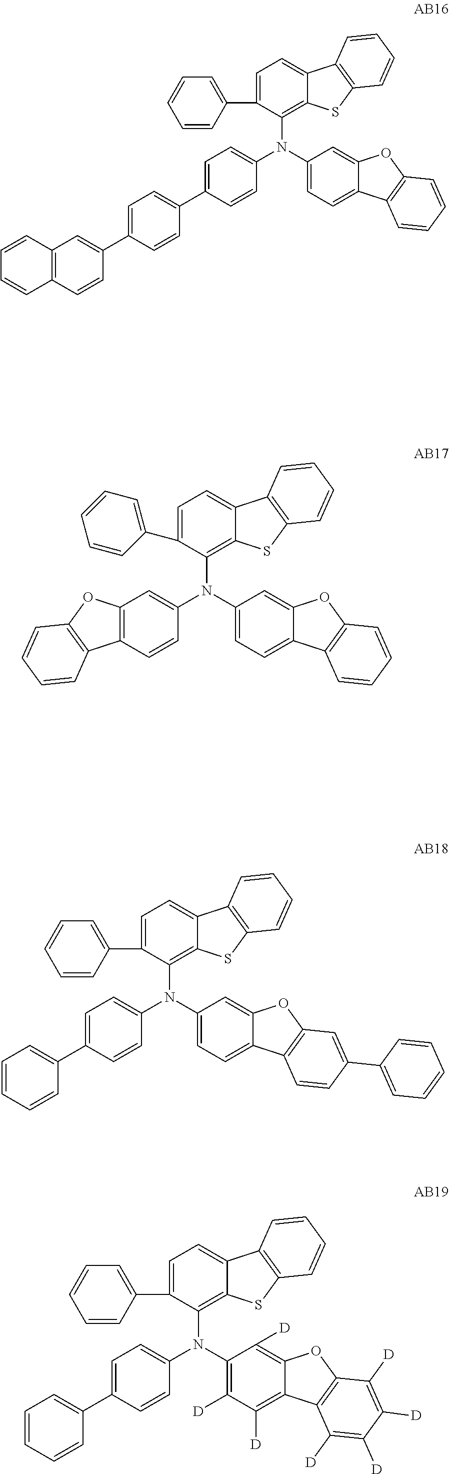 [SMILES: c1ccc(-c2ccc3c(sc4ccccc43)c2N(c2ccc(-c3ccc(-c4ccc5ccccc5c4)cc3)cc2)c2ccc3c(c2)oc2ccccc23)cc1 ; c1ccc(-c2ccc3c(sc4ccccc43)c2N(c2ccc3c(c2)oc2ccccc23)c2ccc3c(c2)oc2ccccc23)cc1 ; c1ccc(-c2ccc(N(c3ccc4c(c3)oc3cc(-c5ccccc5)ccc34)c3c(-c4ccccc4)ccc4c3sc3ccccc34)cc2)cc1 ; [2H]c1c([2H])c([2H])c2c(oc3c([2H])c(N(c4ccc(-c5ccccc5)cc4)c4c(-c5ccccc5)ccc5c4sc4ccccc45)c([2H])c([2H])c32)c1[2H]] 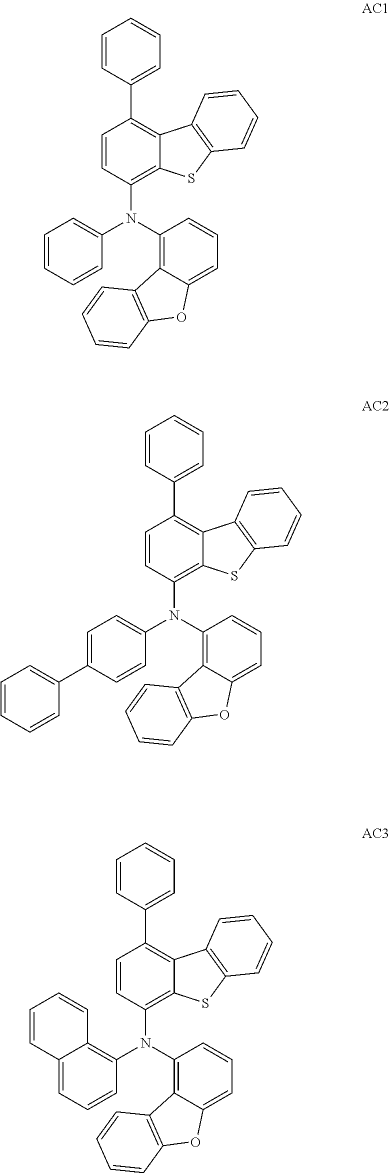 [SMILES: c1ccc(-c2ccc(N(c3ccccc3)c3cccc4oc5ccccc5c34)c3sc4ccccc4c23)cc1 ; c1ccc(-c2ccc(N(c3ccc(-c4ccccc4)c4c3sc3ccccc34)c3cccc4oc5ccccc5c34)cc2)cc1 ; c1ccc(-c2ccc(N(c3cccc4ccccc34)c3cccc4oc5ccccc5c34)c3sc4ccccc4c23)cc1] 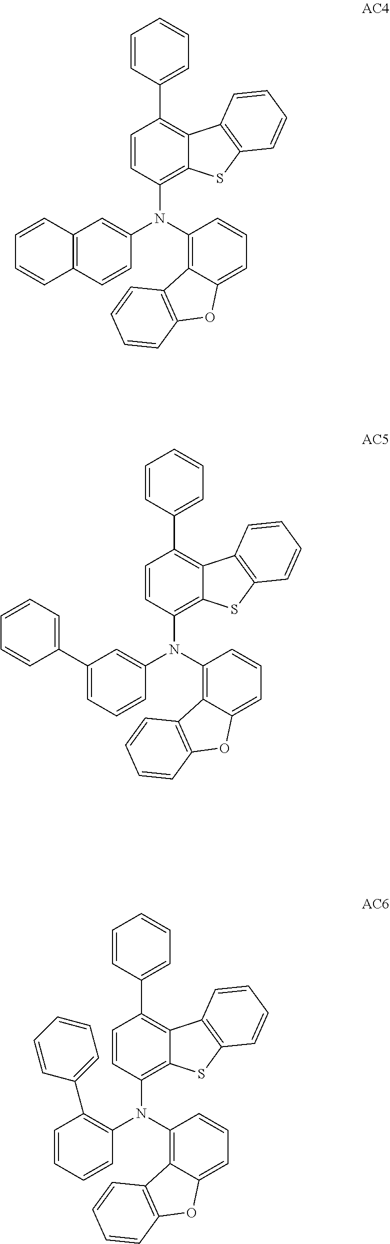 [SMILES: c1ccc(-c2ccc(N(c3ccc4ccccc4c3)c3cccc4oc5ccccc5c34)c3sc4ccccc4c23)cc1 ; c1ccc(-c2cccc(N(c3ccc(-c4ccccc4)c4c3sc3ccccc34)c3cccc4oc5ccccc5c34)c2)cc1 ; c1ccc(-c2ccccc2N(c2ccc(-c3ccccc3)c3c2sc2ccccc23)c2cccc3oc4ccccc4c23)cc1] 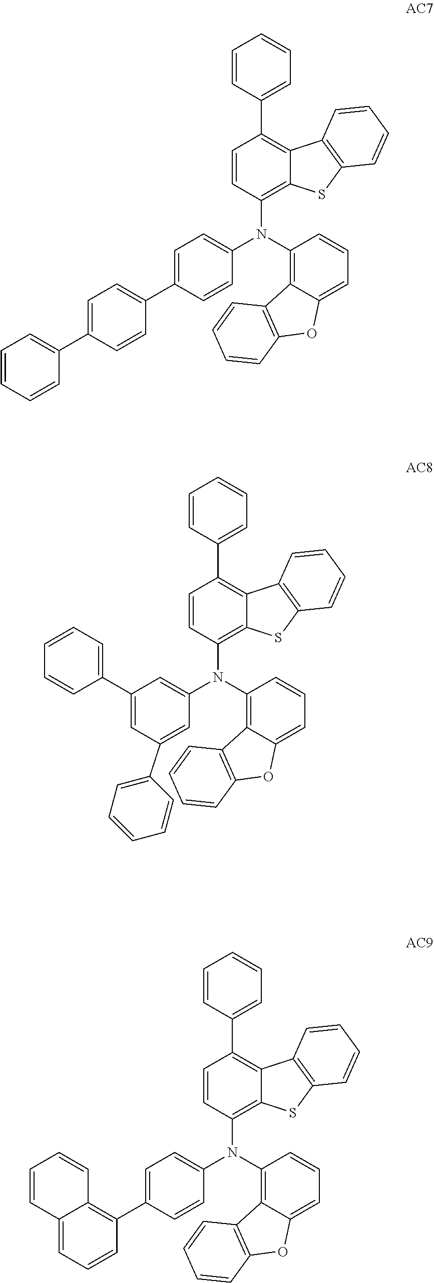 [SMILES: c1ccc(-c2ccc(-c3ccc(N(c4ccc(-c5ccccc5)c5c4sc4ccccc45)c4cccc5oc6ccccc6c45)cc3)cc2)cc1 ; c1ccc(-c2cc(-c3ccccc3)cc(N(c3ccc(-c4ccccc4)c4c3sc3ccccc34)c3cccc4oc5ccccc5c34)c2)cc1 ; c1ccc(-c2ccc(N(c3ccc(-c4cccc5ccccc45)cc3)c3cccc4oc5ccccc5c34)c3sc4ccccc4c23)cc1] 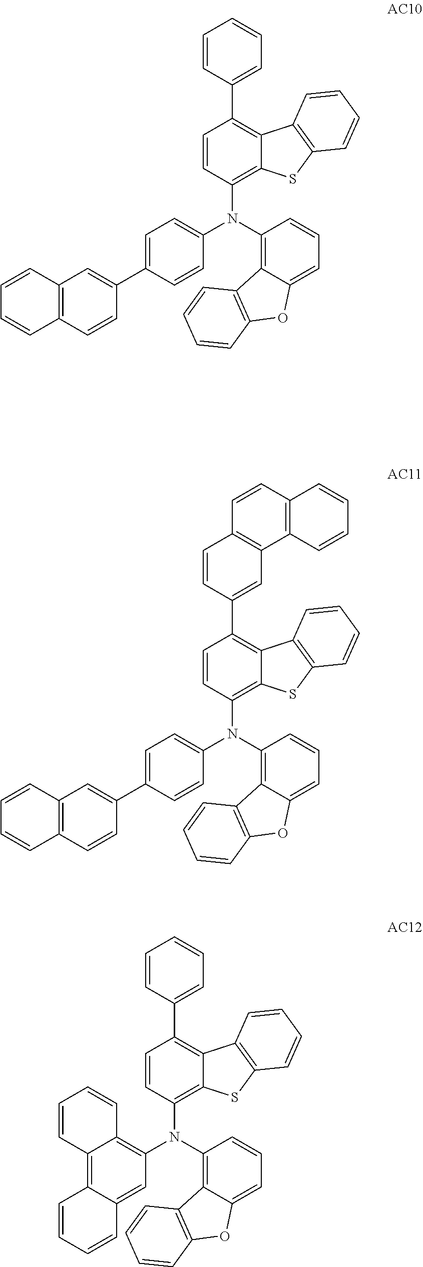 [SMILES: c1ccc(-c2ccc(N(c3ccc(-c4ccc5ccccc5c4)cc3)c3cccc4oc5ccccc5c34)c3sc4ccccc4c23)cc1 ; c1ccc2cc(-c3ccc(N(c4ccc(-c5ccc6ccc7ccccc7c6c5)c5c4sc4ccccc45)c4cccc5oc6ccccc6c45)cc3)ccc2c1 ; c1ccc(-c2ccc(N(c3cc4ccccc4c4ccccc34)c3cccc4oc5ccccc5c34)c3sc4ccccc4c23)cc1] 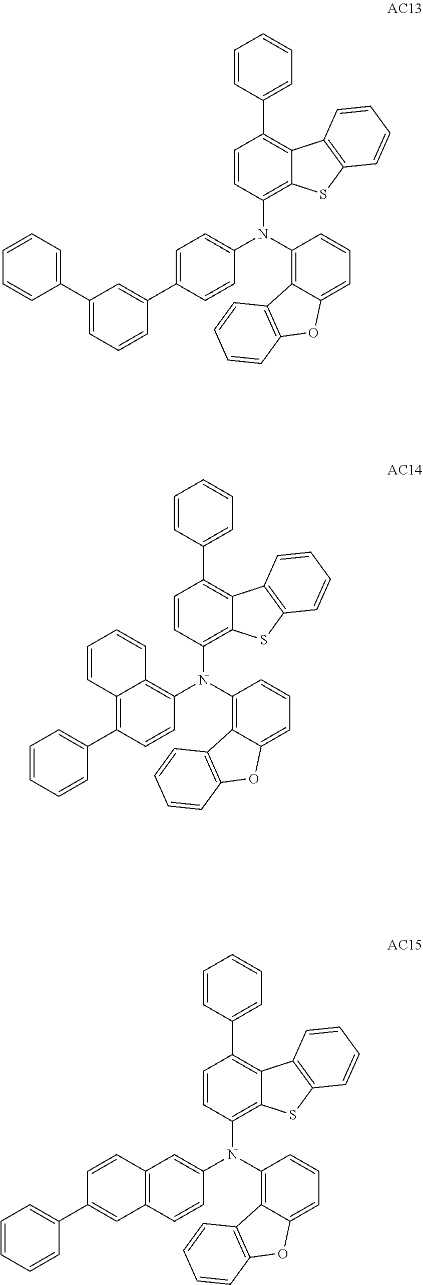 [SMILES: c1ccc(-c2cccc(-c3ccc(N(c4ccc(-c5ccccc5)c5c4sc4ccccc45)c4cccc5oc6ccccc6c45)cc3)c2)cc1 ; c1ccc(-c2ccc(N(c3ccc(-c4ccccc4)c4c3sc3ccccc34)c3cccc4oc5ccccc5c34)c3ccccc23)cc1 ; c1ccc(-c2ccc3cc(N(c4ccc(-c5ccccc5)c5c4sc4ccccc45)c4cccc5oc6ccccc6c45)ccc3c2)cc1] 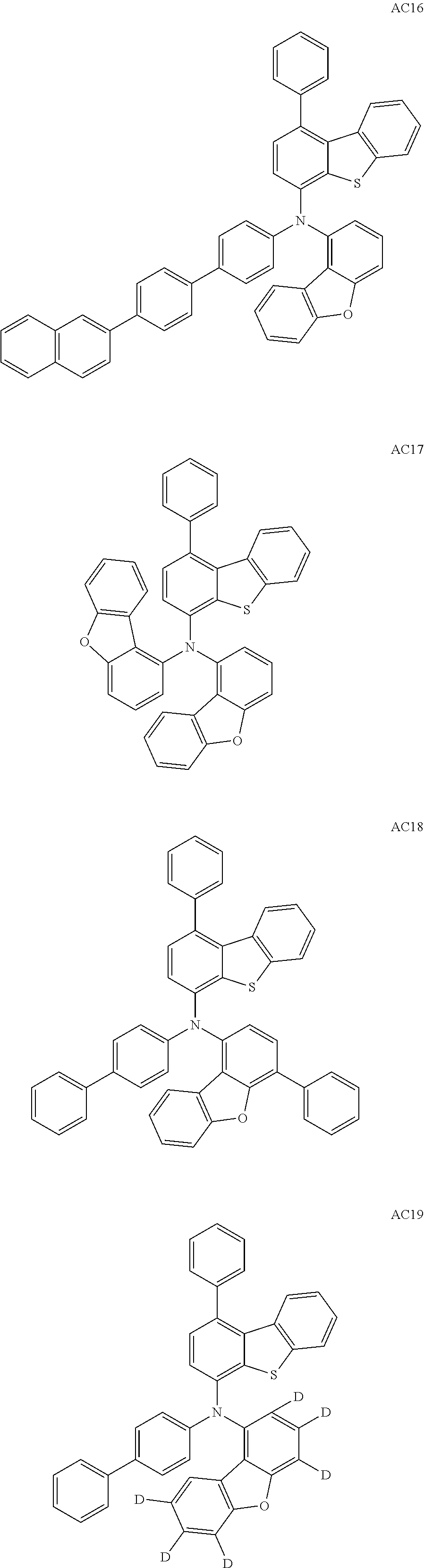 [SMILES: c1ccc(-c2ccc(N(c3ccc(-c4ccc(-c5ccc6ccccc6c5)cc4)cc3)c3cccc4oc5ccccc5c34)c3sc4ccccc4c23)cc1 ; c1ccc(-c2ccc(N(c3cccc4oc5ccccc5c34)c3cccc4oc5ccccc5c34)c3sc4ccccc4c23)cc1 ; c1ccc(-c2ccc(N(c3ccc(-c4ccccc4)c4c3sc3ccccc34)c3ccc(-c4ccccc4)c4oc5ccccc5c34)cc2)cc1 ; [2H]c1cc2c(oc3c([2H])c([2H])c([2H])c(N(c4ccc(-c5ccccc5)cc4)c4ccc(-c5ccccc5)c5c4sc4ccccc45)c32)c([2H])c1[2H]] 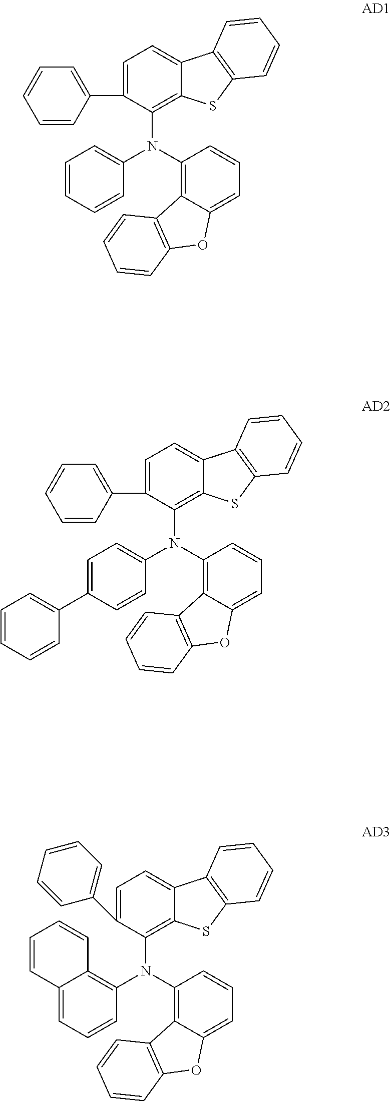 [SMILES: c1ccc(-c2ccc3c(sc4ccccc43)c2N(c2ccccc2)c2cccc3oc4ccccc4c23)cc1 ; c1ccc(-c2ccc(N(c3c(-c4ccccc4)ccc4c3sc3ccccc34)c3cccc4oc5ccccc5c34)cc2)cc1 ; c1ccc(-c2ccc3c(sc4ccccc43)c2N(c2cccc3ccccc23)c2cccc3oc4ccccc4c23)cc1] 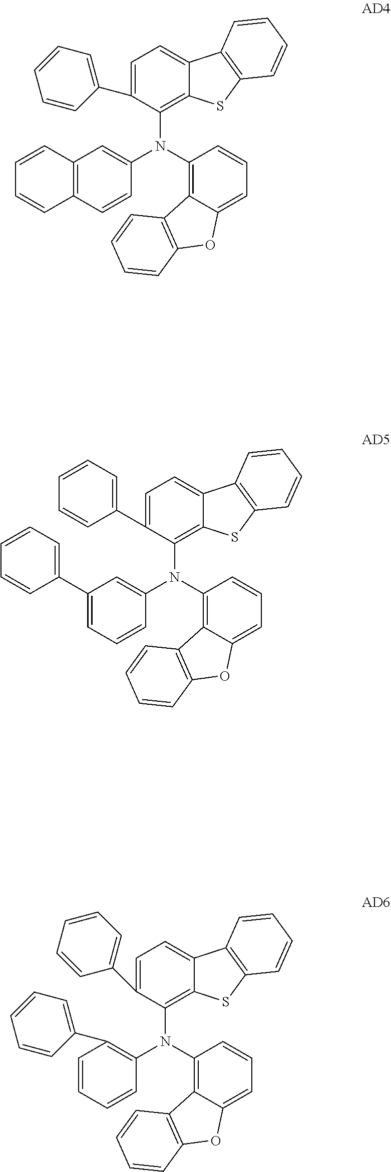 [SMILES: c1ccc(-c2ccc3c(sc4ccccc43)c2N(c2ccc3ccccc3c2)c2cccc3oc4ccccc4c23)cc1 ; c1ccc(-c2cccc(N(c3c(-c4ccccc4)ccc4c3sc3ccccc34)c3cccc4oc5ccccc5c34)c2)cc1 ; c1ccc(-c2ccccc2N(c2c(-c3ccccc3)ccc3c2sc2ccccc23)c2cccc3oc4ccccc4c23)cc1] 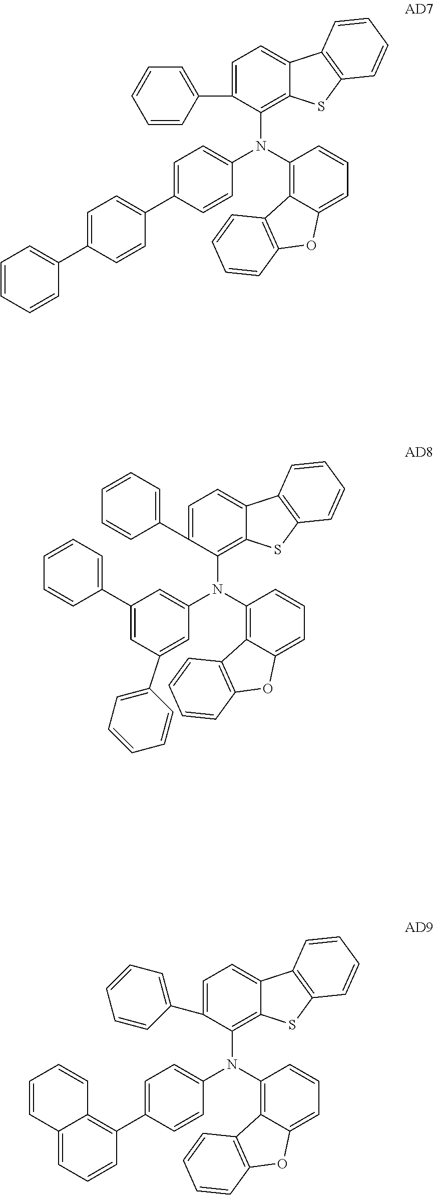 [SMILES: c1ccc(-c2ccc(-c3ccc(N(c4c(-c5ccccc5)ccc5c4sc4ccccc45)c4cccc5oc6ccccc6c45)cc3)cc2)cc1 ; c1ccc(-c2cc(-c3ccccc3)cc(N(c3c(-c4ccccc4)ccc4c3sc3ccccc34)c3cccc4oc5ccccc5c34)c2)cc1 ; c1ccc(-c2ccc3c(sc4ccccc43)c2N(c2ccc(-c3cccc4ccccc34)cc2)c2cccc3oc4ccccc4c23)cc1] 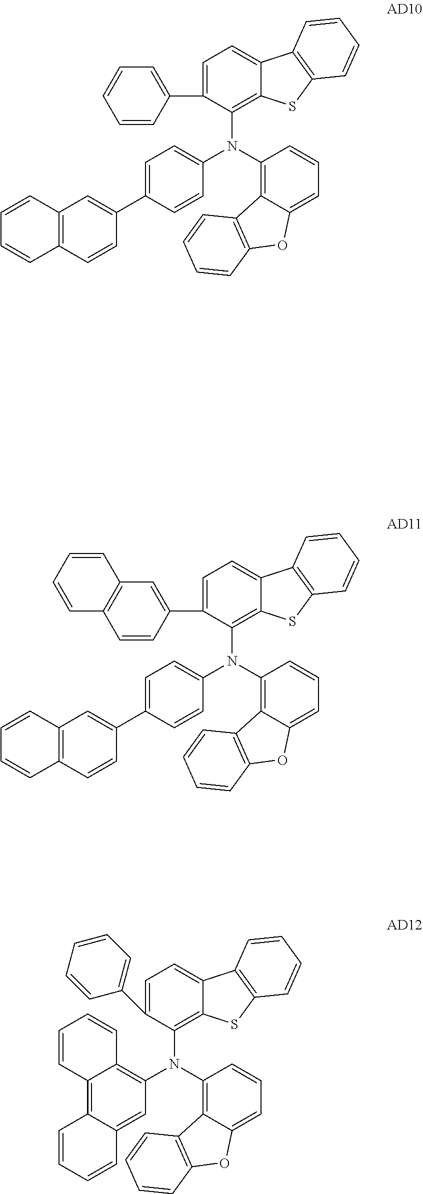 [SMILES: c1ccc(-c2ccc3c(sc4ccccc43)c2N(c2ccc(-c3ccc4ccccc4c3)cc2)c2cccc3oc4ccccc4c23)cc1 ; c1ccc2cc(-c3ccc(N(c4c(-c5ccc6ccccc6c5)ccc5c4sc4ccccc45)c4cccc5oc6ccccc6c45)cc3)ccc2c1 ; c1ccc(-c2ccc3c(sc4ccccc43)c2N(c2cc3ccccc3c3ccccc23)c2cccc3oc4ccccc4c23)cc1] 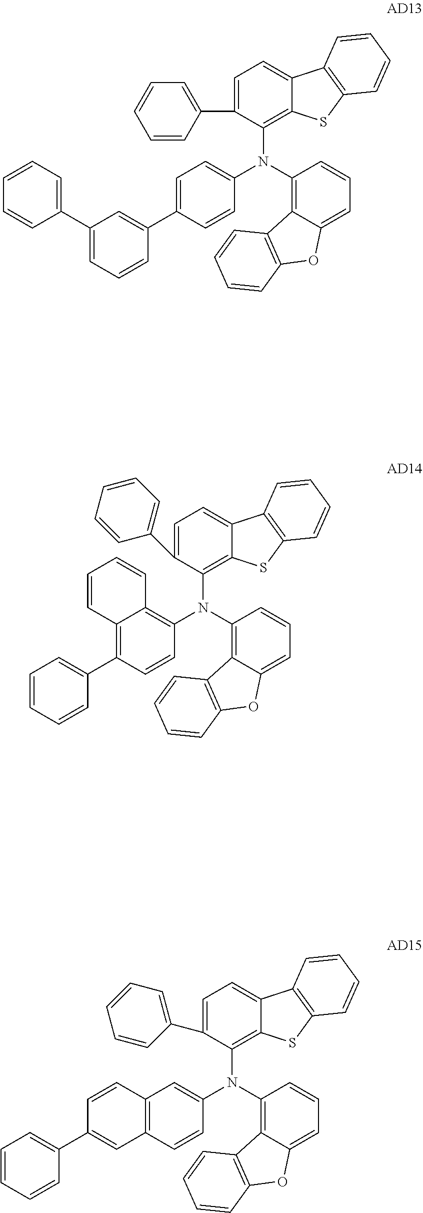 [SMILES: c1ccc(-c2cccc(-c3ccc(N(c4c(-c5ccccc5)ccc5c4sc4ccccc45)c4cccc5oc6ccccc6c45)cc3)c2)cc1 ; c1ccc(-c2ccc3c(sc4ccccc43)c2N(c2ccc(-c3ccccc3)c3ccccc23)c2cccc3oc4ccccc4c23)cc1 ; c1ccc(-c2ccc3cc(N(c4c(-c5ccccc5)ccc5c4sc4ccccc45)c4cccc5oc6ccccc6c45)ccc3c2)cc1] 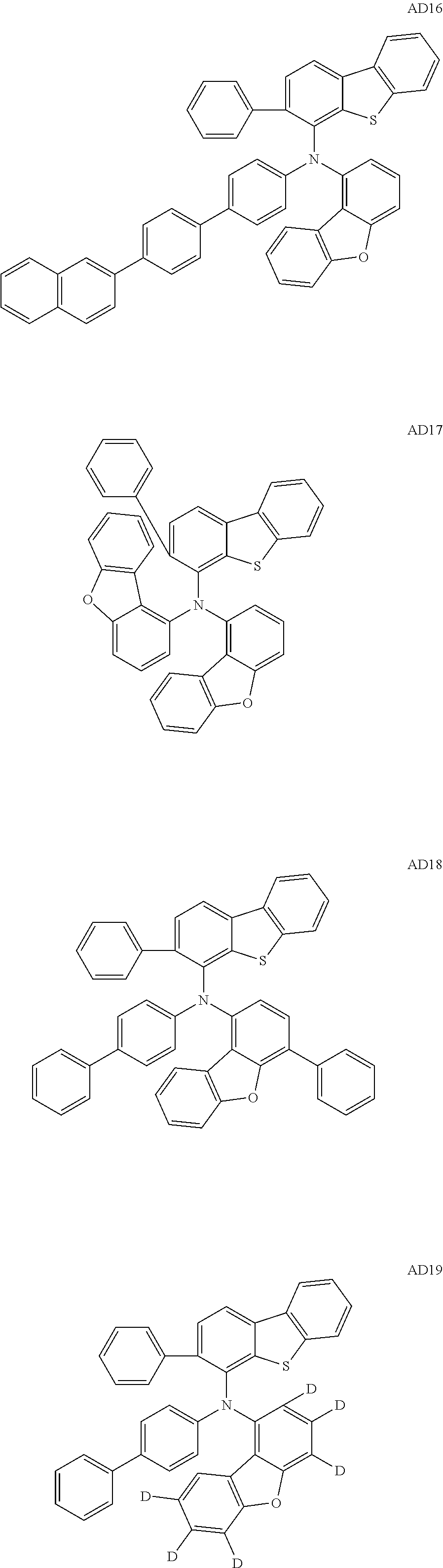 [SMILES: c1ccc(-c2ccc3c(sc4ccccc43)c2N(c2ccc(-c3ccc(-c4ccc5ccccc5c4)cc3)cc2)c2cccc3oc4ccccc4c23)cc1 ; c1ccc(-c2ccc3c(sc4ccccc43)c2N(c2cccc3oc4ccccc4c23)c2cccc3oc4ccccc4c23)cc1 ; c1ccc(-c2ccc(N(c3c(-c4ccccc4)ccc4c3sc3ccccc34)c3ccc(-c4ccccc4)c4oc5ccccc5c34)cc2)cc1 ; [2H]c1cc2c(oc3c([2H])c([2H])c([2H])c(N(c4ccc(-c5ccccc5)cc4)c4c(-c5ccccc5)ccc5c4sc4ccccc45)c32)c([2H])c1[2H]] 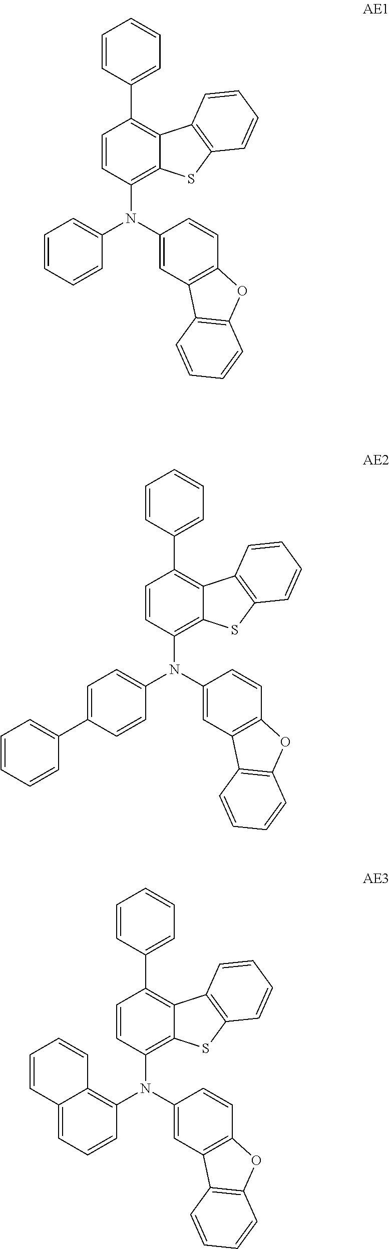 [SMILES: c1ccc(-c2ccc(N(c3ccccc3)c3ccc4oc5ccccc5c4c3)c3sc4ccccc4c23)cc1 ; c1ccc(-c2ccc(N(c3ccc4oc5ccccc5c4c3)c3ccc(-c4ccccc4)c4c3sc3ccccc34)cc2)cc1 ; c1ccc(-c2ccc(N(c3ccc4oc5ccccc5c4c3)c3cccc4ccccc34)c3sc4ccccc4c23)cc1] 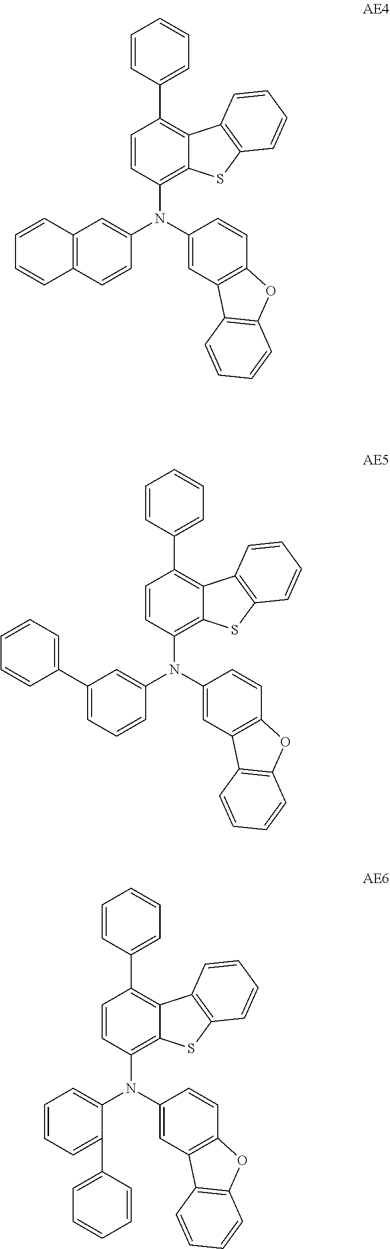 [SMILES: c1ccc(-c2ccc(N(c3ccc4ccccc4c3)c3ccc4oc5ccccc5c4c3)c3sc4ccccc4c23)cc1 ; c1ccc(-c2cccc(N(c3ccc4oc5ccccc5c4c3)c3ccc(-c4ccccc4)c4c3sc3ccccc34)c2)cc1 ; c1ccc(-c2ccccc2N(c2ccc3oc4ccccc4c3c2)c2ccc(-c3ccccc3)c3c2sc2ccccc23)cc1] 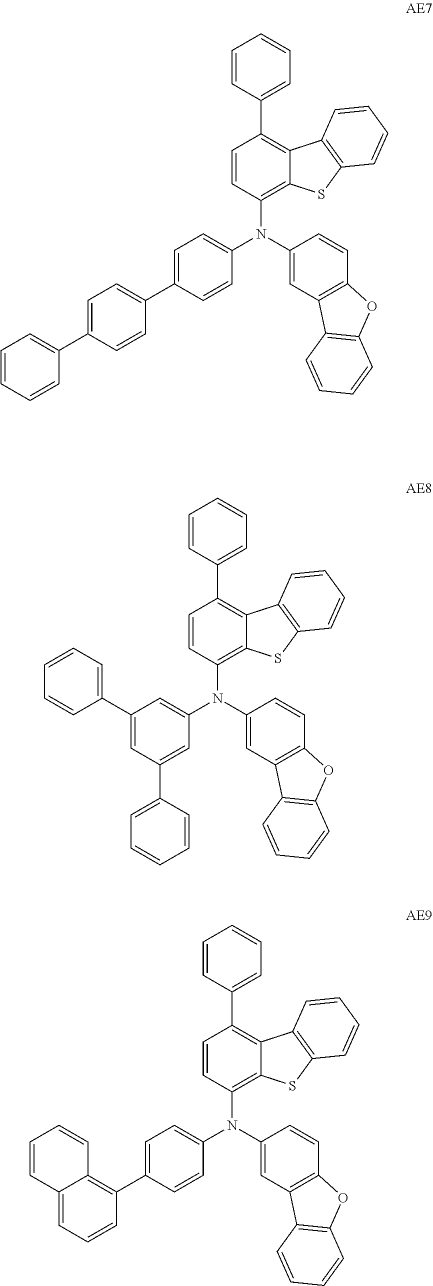 [SMILES: c1ccc(-c2ccc(-c3ccc(N(c4ccc5oc6ccccc6c5c4)c4ccc(-c5ccccc5)c5c4sc4ccccc45)cc3)cc2)cc1 ; c1ccc(-c2cc(-c3ccccc3)cc(N(c3ccc4oc5ccccc5c4c3)c3ccc(-c4ccccc4)c4c3sc3ccccc34)c2)cc1 ; c1ccc(-c2ccc(N(c3ccc(-c4cccc5ccccc45)cc3)c3ccc4oc5ccccc5c4c3)c3sc4ccccc4c23)cc1] 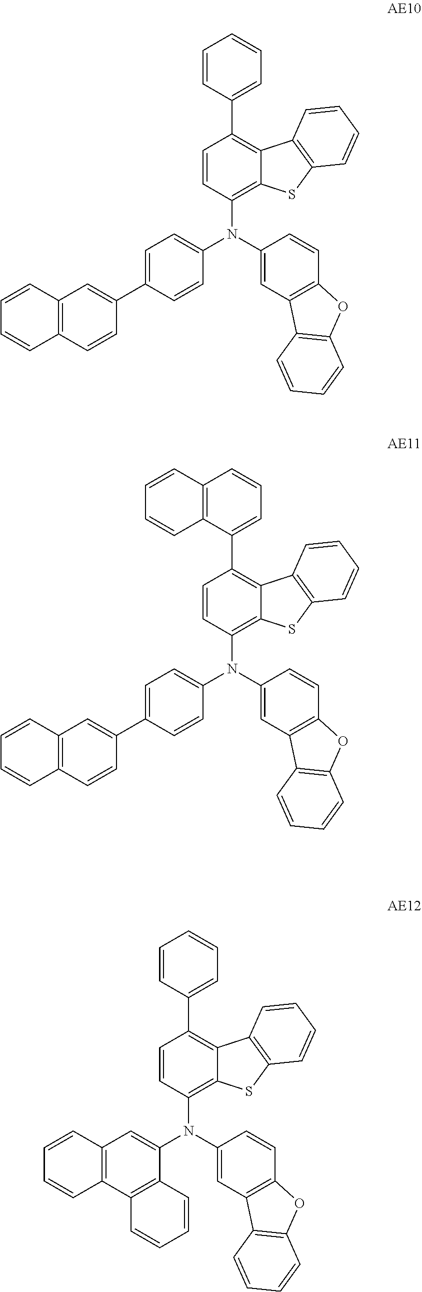 [SMILES: c1ccc(-c2ccc(N(c3ccc(-c4ccc5ccccc5c4)cc3)c3ccc4oc5ccccc5c4c3)c3sc4ccccc4c23)cc1 ; c1ccc2cc(-c3ccc(N(c4ccc5oc6ccccc6c5c4)c4ccc(-c5cccc6ccccc56)c5c4sc4ccccc45)cc3)ccc2c1 ; c1ccc(-c2ccc(N(c3ccc4oc5ccccc5c4c3)c3cc4ccccc4c4ccccc34)c3sc4ccccc4c23)cc1] 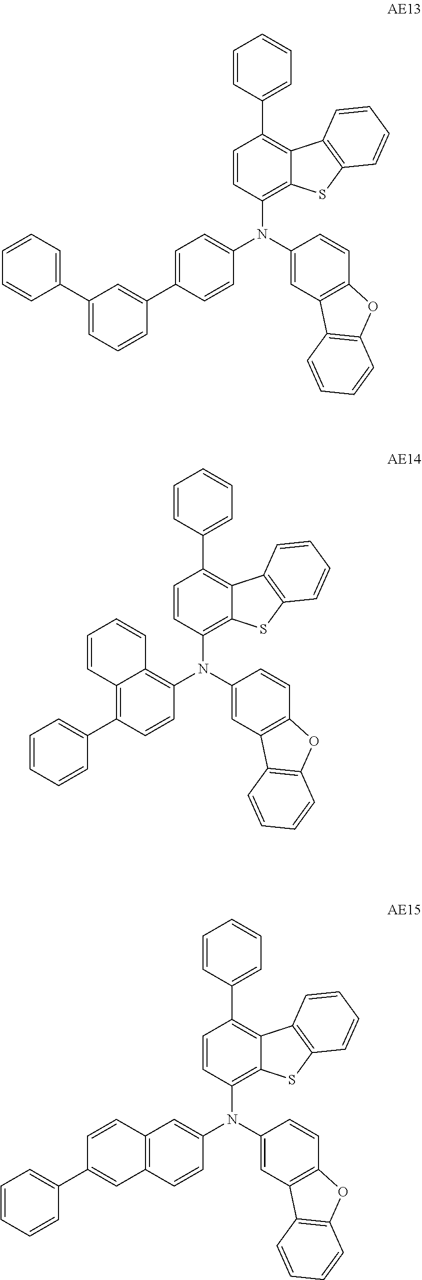 [SMILES: c1ccc(-c2cccc(-c3ccc(N(c4ccc5oc6ccccc6c5c4)c4ccc(-c5ccccc5)c5c4sc4ccccc45)cc3)c2)cc1 ; c1ccc(-c2ccc(N(c3ccc4oc5ccccc5c4c3)c3ccc(-c4ccccc4)c4c3sc3ccccc34)c3ccccc23)cc1 ; c1ccc(-c2ccc3cc(N(c4ccc5oc6ccccc6c5c4)c4ccc(-c5ccccc5)c5c4sc4ccccc45)ccc3c2)cc1] 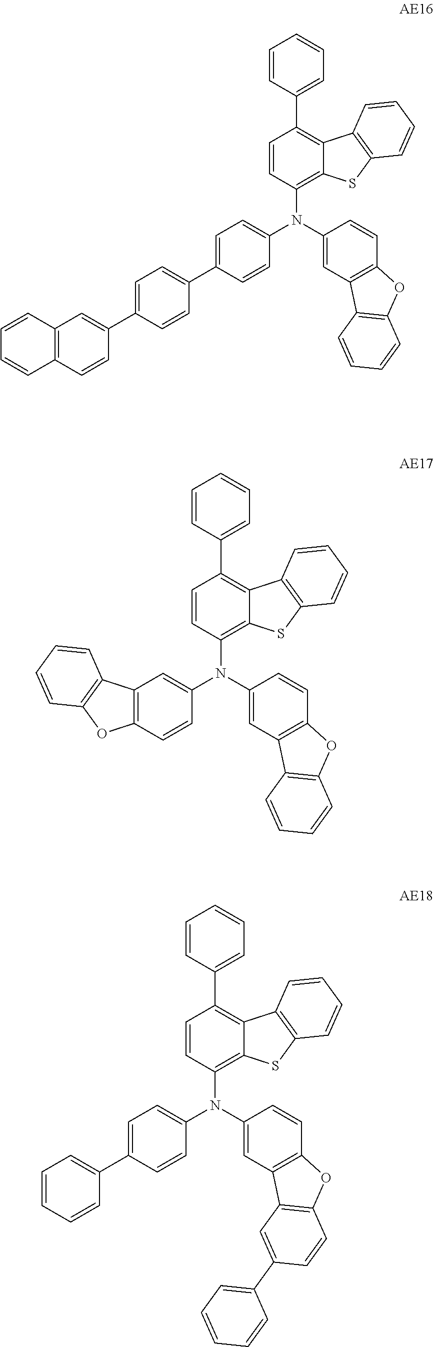 [SMILES: c1ccc(-c2ccc(N(c3ccc(-c4ccc(-c5ccc6ccccc6c5)cc4)cc3)c3ccc4oc5ccccc5c4c3)c3sc4ccccc4c23)cc1 ; c1ccc(-c2ccc(N(c3ccc4oc5ccccc5c4c3)c3ccc4oc5ccccc5c4c3)c3sc4ccccc4c23)cc1 ; c1ccc(-c2ccc(N(c3ccc4oc5ccc(-c6ccccc6)cc5c4c3)c3ccc(-c4ccccc4)c4c3sc3ccccc34)cc2)cc1] 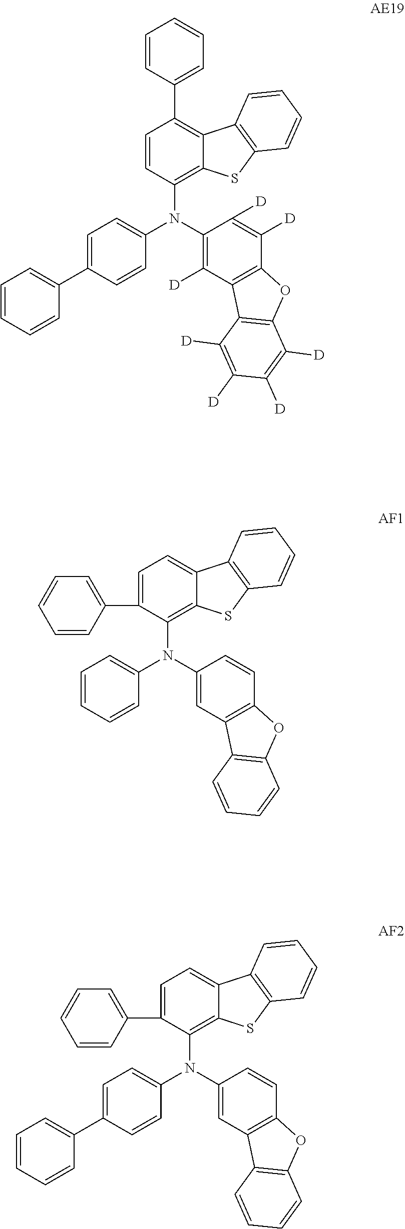 [SMILES: [2H]c1c([2H])c([2H])c2c(oc3c([2H])c([2H])c(N(c4ccc(-c5ccccc5)cc4)c4ccc(-c5ccccc5)c5c4sc4ccccc45)c([2H])c32)c1[2H] ; c1ccc(-c2ccc3c(sc4ccccc43)c2N(c2ccccc2)c2ccc3oc4ccccc4c3c2)cc1 ; c1ccc(-c2ccc(N(c3ccc4oc5ccccc5c4c3)c3c(-c4ccccc4)ccc4c3sc3ccccc34)cc2)cc1] 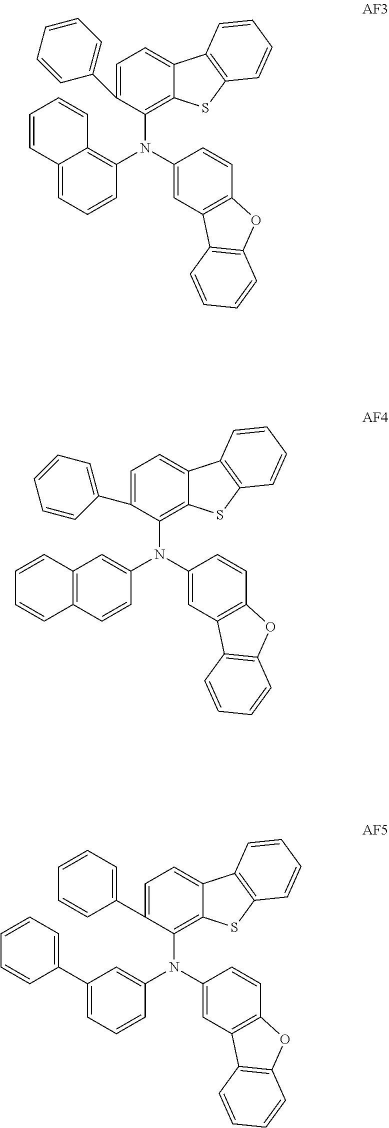 [SMILES: c1ccc(-c2ccc3c(sc4ccccc43)c2N(c2ccc3oc4ccccc4c3c2)c2cccc3ccccc23)cc1 ; c1ccc(-c2ccc3c(sc4ccccc43)c2N(c2ccc3ccccc3c2)c2ccc3oc4ccccc4c3c2)cc1 ; c1ccc(-c2cccc(N(c3ccc4oc5ccccc5c4c3)c3c(-c4ccccc4)ccc4c3sc3ccccc34)c2)cc1] 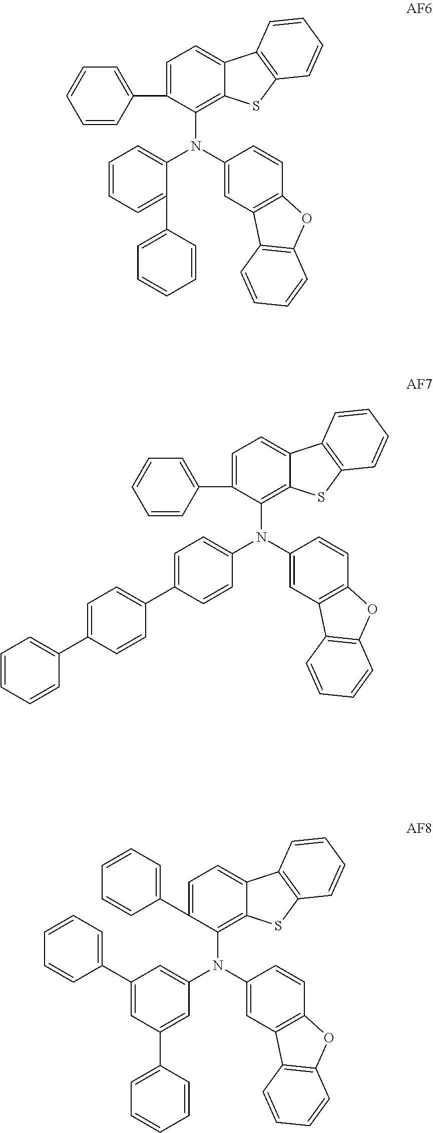 [SMILES: c1ccc(-c2ccccc2N(c2ccc3oc4ccccc4c3c2)c2c(-c3ccccc3)ccc3c2sc2ccccc23)cc1 ; c1ccc(-c2ccc(-c3ccc(N(c4ccc5oc6ccccc6c5c4)c4c(-c5ccccc5)ccc5c4sc4ccccc45)cc3)cc2)cc1 ; c1ccc(-c2cc(-c3ccccc3)cc(N(c3ccc4oc5ccccc5c4c3)c3c(-c4ccccc4)ccc4c3sc3ccccc34)c2)cc1] 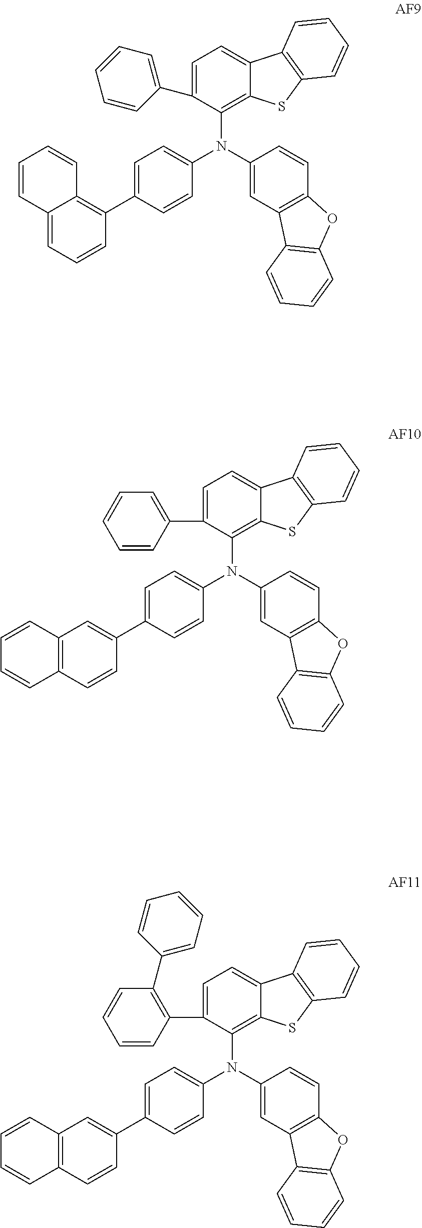 [SMILES: c1ccc(-c2ccc3c(sc4ccccc43)c2N(c2ccc(-c3cccc4ccccc34)cc2)c2ccc3oc4ccccc4c3c2)cc1 ; c1ccc(-c2ccc3c(sc4ccccc43)c2N(c2ccc(-c3ccc4ccccc4c3)cc2)c2ccc3oc4ccccc4c3c2)cc1 ; c1ccc(-c2ccccc2-c2ccc3c(sc4ccccc43)c2N(c2ccc(-c3ccc4ccccc4c3)cc2)c2ccc3oc4ccccc4c3c2)cc1] 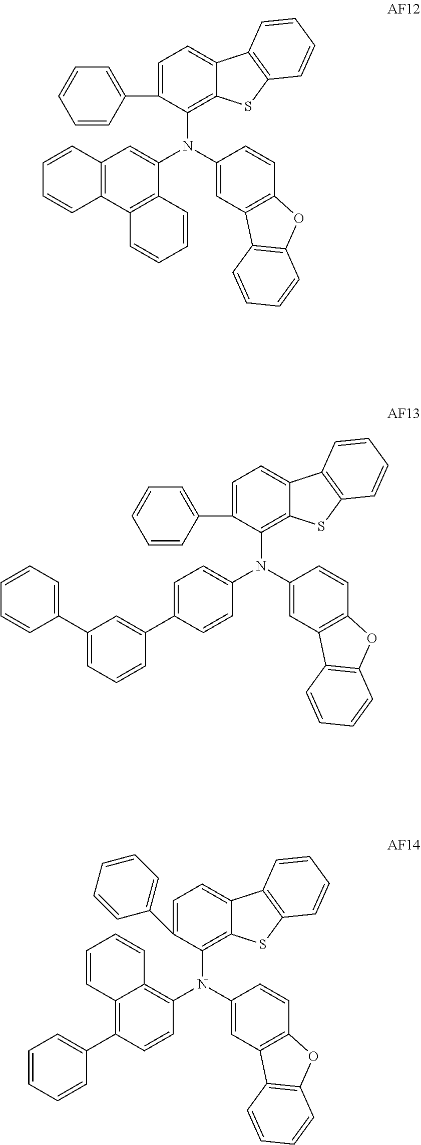 [SMILES: c1ccc(-c2ccc3c(sc4ccccc43)c2N(c2ccc3oc4ccccc4c3c2)c2cc3ccccc3c3ccccc23)cc1 ; c1ccc(-c2cccc(-c3ccc(N(c4ccc5oc6ccccc6c5c4)c4c(-c5ccccc5)ccc5c4sc4ccccc45)cc3)c2)cc1 ; c1ccc(-c2ccc3c(sc4ccccc43)c2N(c2ccc3oc4ccccc4c3c2)c2ccc(-c3ccccc3)c3ccccc23)cc1] 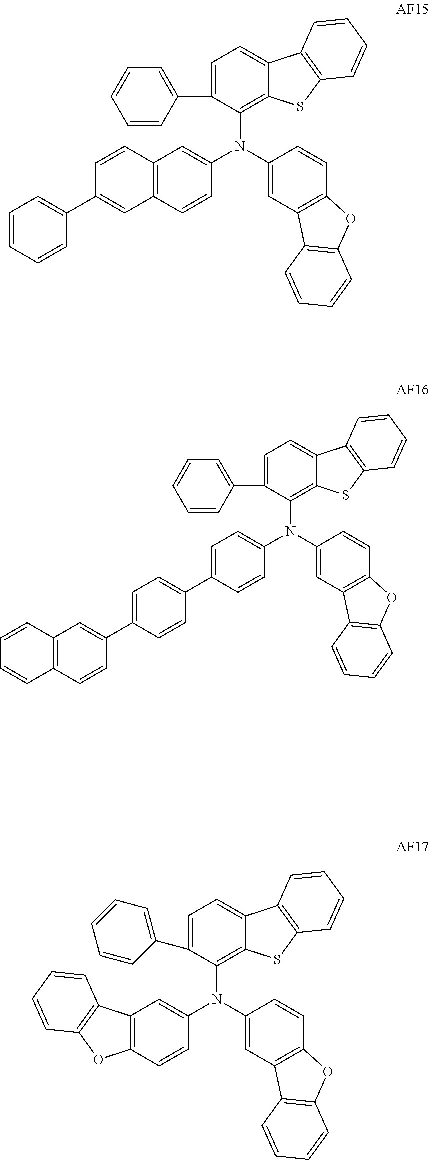 [SMILES: c1ccc(-c2ccc3cc(N(c4ccc5oc6ccccc6c5c4)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc3oc4ccccc4c3c2)cc1 ; c1ccc(-c2ccc3c(sc4ccccc43)c2N(c2ccc3oc4ccccc4c3c2)c2ccc3oc4ccccc4c3c2)cc1] 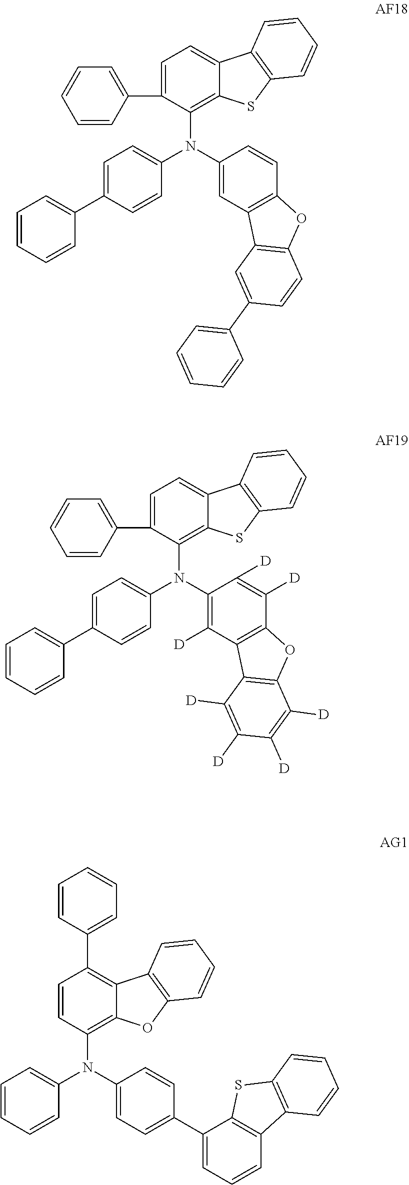 [SMILES: c1ccc(-c2ccc(N(c3ccc4oc5ccc(-c6ccccc6)cc5c4c3)c3c(-c4ccccc4)ccc4c3sc3ccccc34)cc2)cc1 ; [2H]c1c([2H])c([2H])c2c(oc3c([2H])c([2H])c(N(c4ccc(-c5ccccc5)cc4)c4c(-c5ccccc5)ccc5c4sc4ccccc45)c([2H])c32)c1[2H] ; c1ccc(-c2ccc(N(c3ccccc3)c3ccc(-c4cccc5c4sc4ccccc45)cc3)c3oc4ccccc4c23)cc1] 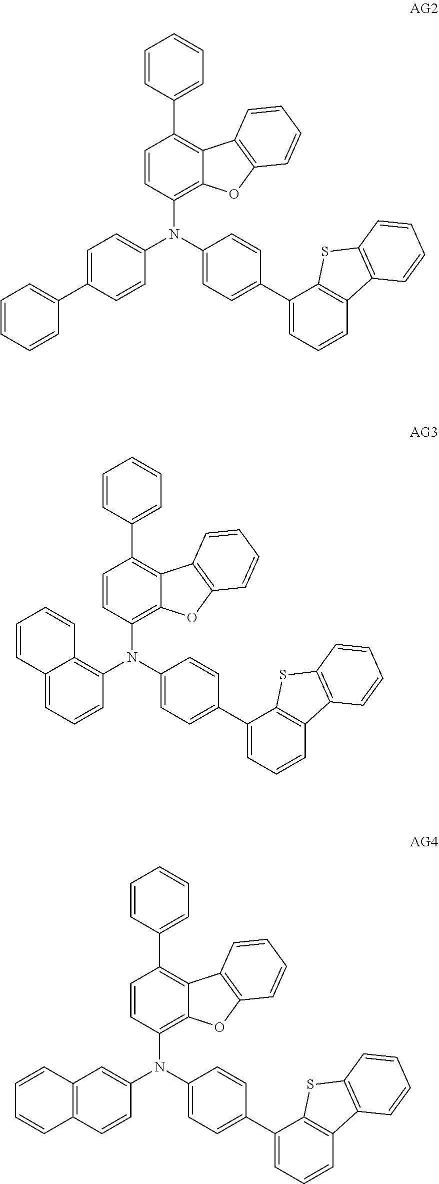 [SMILES: c1ccc(-c2ccc(N(c3ccc(-c4cccc5c4sc4ccccc45)cc3)c3ccc(-c4ccccc4)c4c3oc3ccccc34)cc2)cc1 ; c1ccc(-c2ccc(N(c3ccc(-c4cccc5c4sc4ccccc45)cc3)c3cccc4ccccc34)c3oc4ccccc4c23)cc1 ; c1ccc(-c2ccc(N(c3ccc(-c4cccc5c4sc4ccccc45)cc3)c3ccc4ccccc4c3)c3oc4ccccc4c23)cc1] 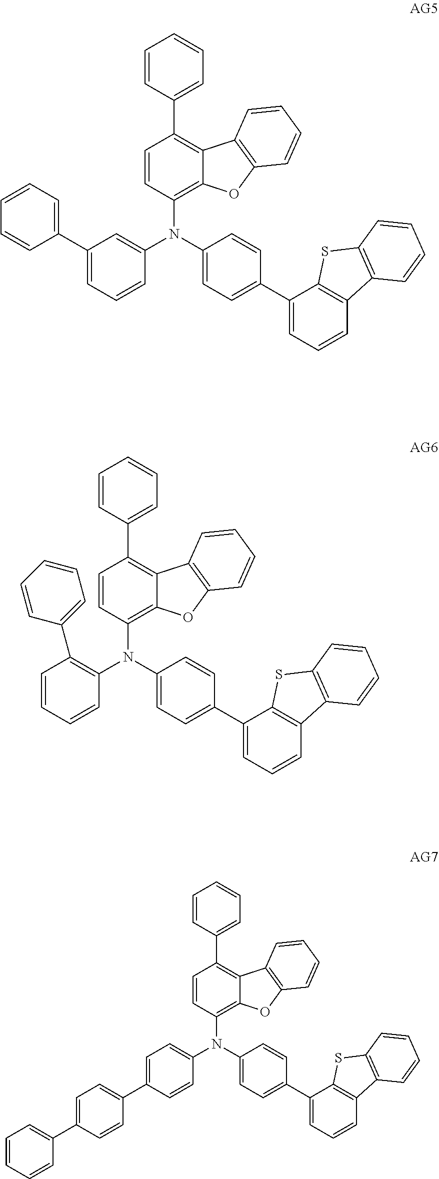 [SMILES: c1ccc(-c2cccc(N(c3ccc(-c4cccc5c4sc4ccccc45)cc3)c3ccc(-c4ccccc4)c4c3oc3ccccc34)c2)cc1 ; c1ccc(-c2ccccc2N(c2ccc(-c3cccc4c3sc3ccccc34)cc2)c2ccc(-c3ccccc3)c3c2oc2ccccc23)cc1 ; c1ccc(-c2ccc(-c3ccc(N(c4ccc(-c5cccc6c5sc5ccccc56)cc4)c4ccc(-c5ccccc5)c5c4oc4ccccc45)cc3)cc2)cc1] 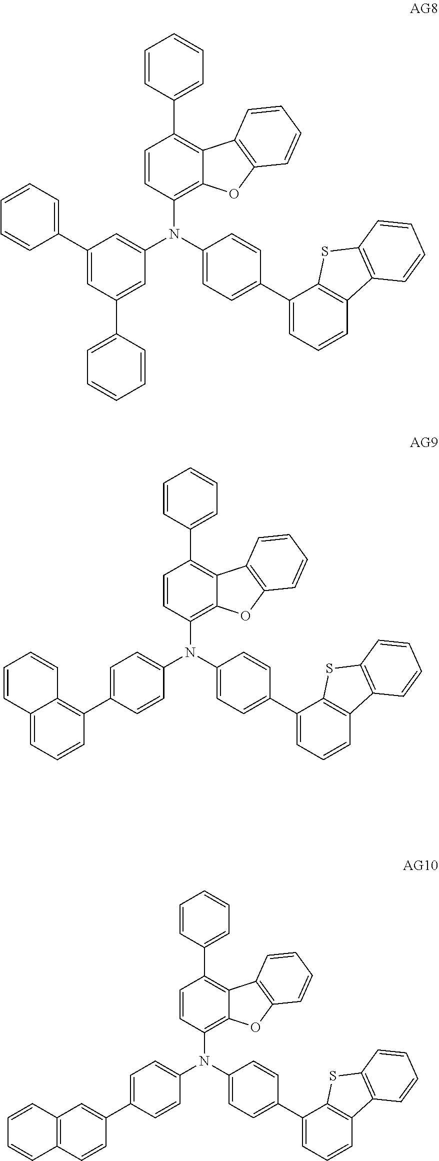 [SMILES: c1ccc(-c2cc(-c3ccccc3)cc(N(c3ccc(-c4cccc5c4sc4ccccc45)cc3)c3ccc(-c4ccccc4)c4c3oc3ccccc34)c2)cc1 ; c1ccc(-c2ccc(N(c3ccc(-c4cccc5ccccc45)cc3)c3ccc(-c4cccc5c4sc4ccccc45)cc3)c3oc4ccccc4c23)cc1 ; c1ccc(-c2ccc(N(c3ccc(-c4ccc5ccccc5c4)cc3)c3ccc(-c4cccc5c4sc4ccccc45)cc3)c3oc4ccccc4c23)cc1] 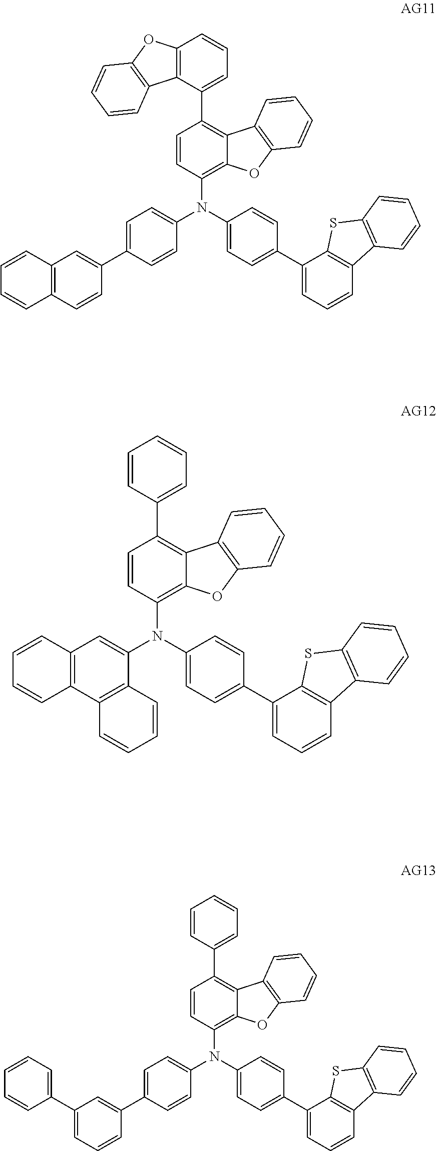 [SMILES: c1ccc2cc(-c3ccc(N(c4ccc(-c5cccc6c5sc5ccccc56)cc4)c4ccc(-c5cccc6oc7ccccc7c56)c5c4oc4ccccc45)cc3)ccc2c1 ; c1ccc(-c2ccc(N(c3ccc(-c4cccc5c4sc4ccccc45)cc3)c3cc4ccccc4c4ccccc34)c3oc4ccccc4c23)cc1 ; c1ccc(-c2cccc(-c3ccc(N(c4ccc(-c5cccc6c5sc5ccccc56)cc4)c4ccc(-c5ccccc5)c5c4oc4ccccc45)cc3)c2)cc1] 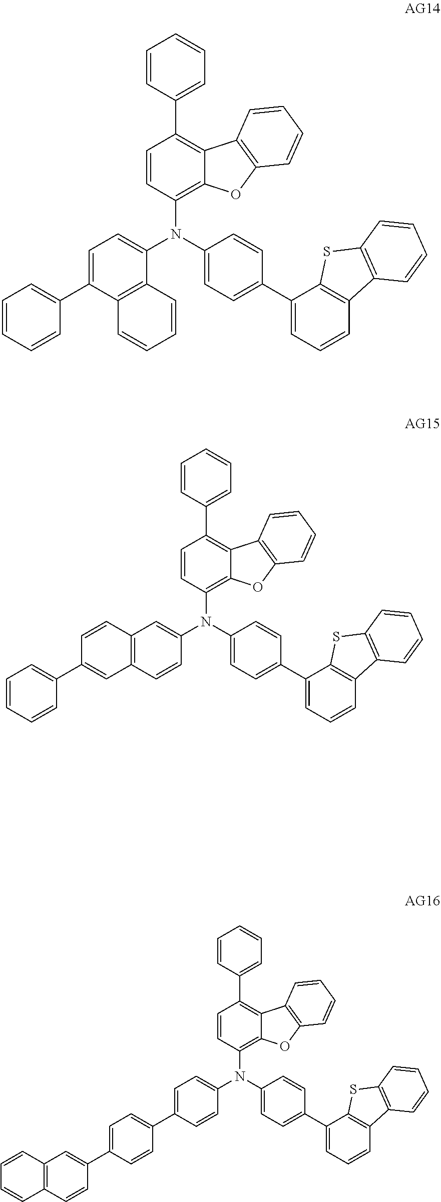 [SMILES: c1ccc(-c2ccc(N(c3ccc(-c4cccc5c4sc4ccccc45)cc3)c3ccc(-c4ccccc4)c4c3oc3ccccc34)c3ccccc23)cc1 ; c1ccc(-c2ccc3cc(N(c4ccc(-c5cccc6c5sc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4cccc5c4sc4ccccc45)cc3)c3oc4ccccc4c23)cc1] 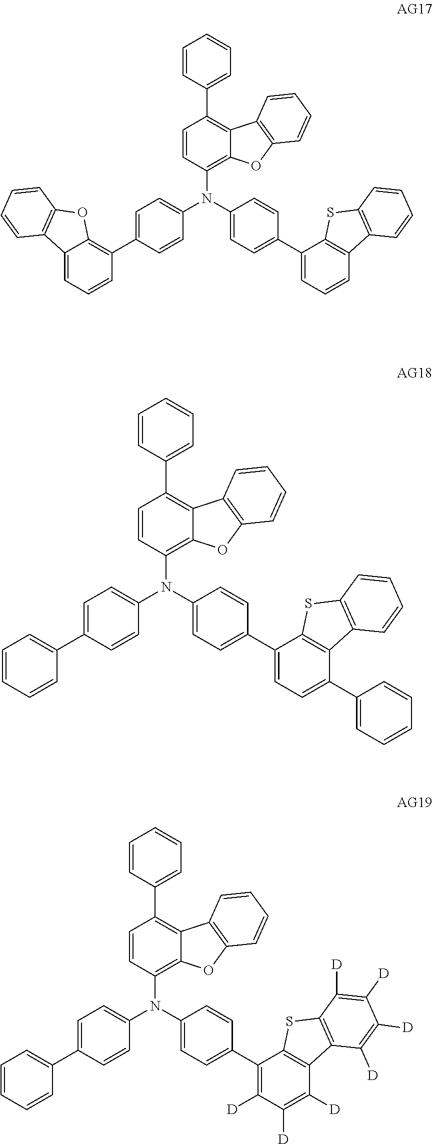 [SMILES: c1ccc(-c2ccc(N(c3ccc(-c4cccc5c4oc4ccccc45)cc3)c3ccc(-c4cccc5c4sc4ccccc45)cc3)c3oc4ccccc4c23)cc1 ; c1ccc(-c2ccc(N(c3ccc(-c4ccc(-c5ccccc5)c5c4sc4ccccc45)cc3)c3ccc(-c4ccccc4)c4c3oc3ccccc34)cc2)cc1 ; [2H]c1c([2H])c([2H])c2c(sc3c(-c4ccc(N(c5ccc(-c6ccccc6)cc5)c5ccc(-c6ccccc6)c6c5oc5ccccc56)cc4)c([2H])c([2H])c([2H])c32)c1[2H]] 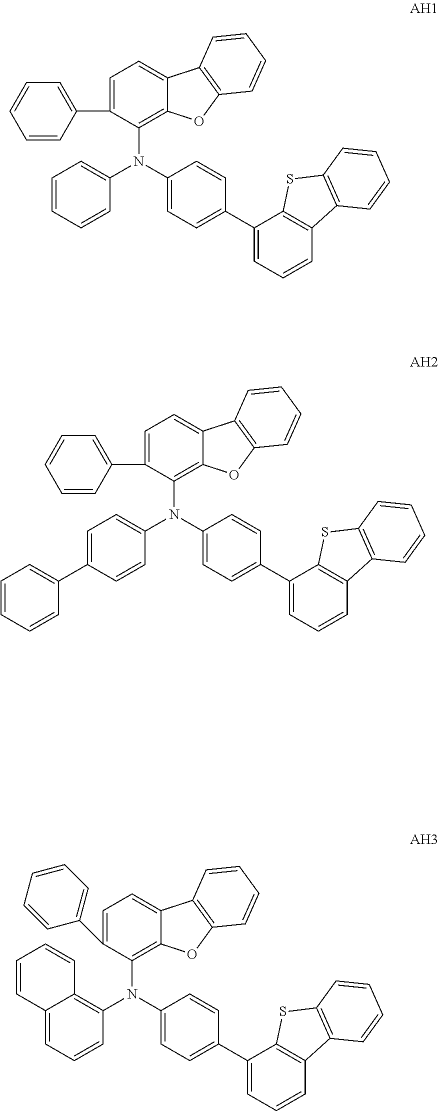 [SMILES: c1ccc(-c2ccc3c(oc4ccccc43)c2N(c2ccccc2)c2ccc(-c3cccc4c3sc3ccccc34)cc2)cc1 ; c1ccc(-c2ccc(N(c3ccc(-c4cccc5c4sc4ccccc45)cc3)c3c(-c4ccccc4)ccc4c3oc3ccccc34)cc2)cc1 ; c1ccc(-c2ccc3c(oc4ccccc43)c2N(c2ccc(-c3cccc4c3sc3ccccc34)cc2)c2cccc3ccccc23)cc1] 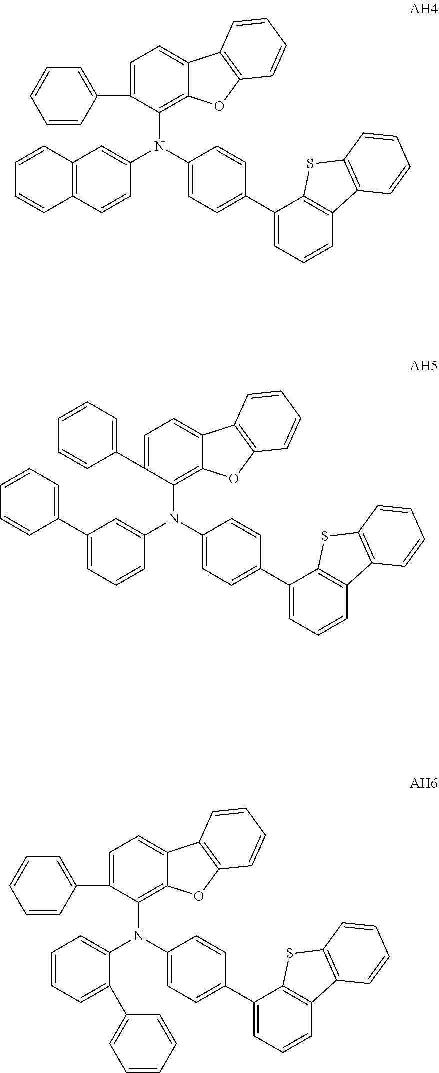 [SMILES: c1ccc(-c2ccc3c(oc4ccccc43)c2N(c2ccc(-c3cccc4c3sc3ccccc34)cc2)c2ccc3ccccc3c2)cc1 ; c1ccc(-c2cccc(N(c3ccc(-c4cccc5c4sc4ccccc45)cc3)c3c(-c4ccccc4)ccc4c3oc3ccccc34)c2)cc1 ; c1ccc(-c2ccccc2N(c2ccc(-c3cccc4c3sc3ccccc34)cc2)c2c(-c3ccccc3)ccc3c2oc2ccccc23)cc1] 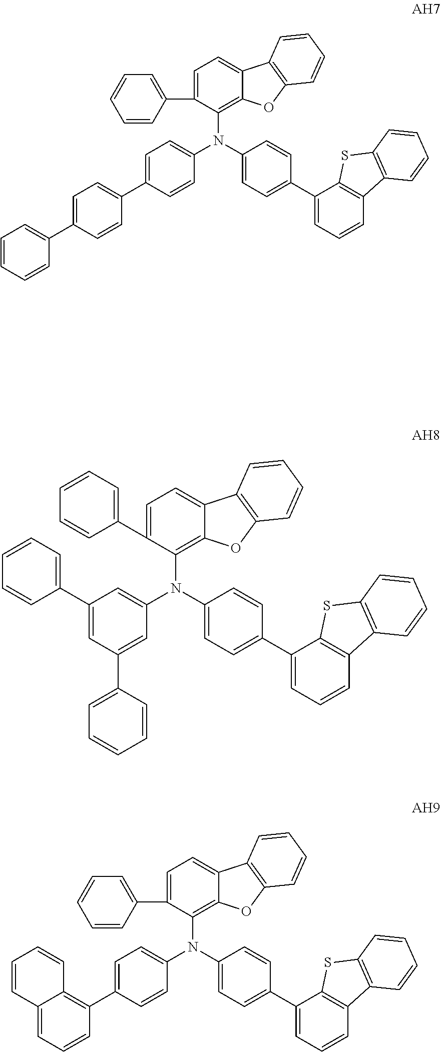 [SMILES: c1ccc(-c2ccc(-c3ccc(N(c4ccc(-c5cccc6c5sc5ccccc56)cc4)c4c(-c5ccccc5)ccc5c4oc4ccccc45)cc3)cc2)cc1 ; c1ccc(-c2cc(-c3ccccc3)cc(N(c3ccc(-c4cccc5c4sc4ccccc45)cc3)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc(-c3cccc4c3sc3ccccc34)cc2)cc1] 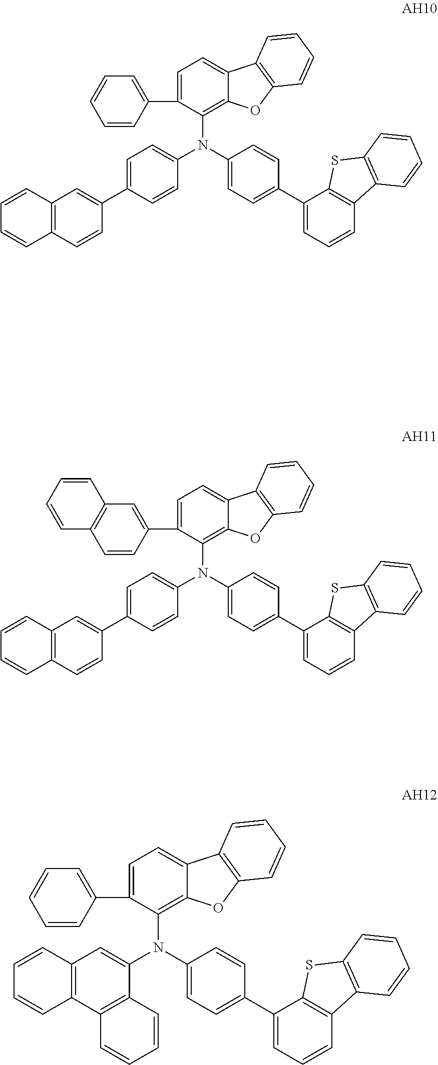 [SMILES: c1ccc(-c2ccc3c(oc4ccccc43)c2N(c2ccc(-c3ccc4ccccc4c3)cc2)c2ccc(-c3cccc4c3sc3ccccc34)cc2)cc1 ; c1ccc2cc(-c3ccc(N(c4ccc(-c5cccc6c5sc5ccccc56)cc4)c4c(-c5ccc6ccccc6c5)ccc5c4oc4ccccc45)cc3)ccc2c1 ; c1ccc(-c2ccc3c(oc4ccccc43)c2N(c2ccc(-c3cccc4c3sc3ccccc34)cc2)c2cc3ccccc3c3ccccc23)cc1] 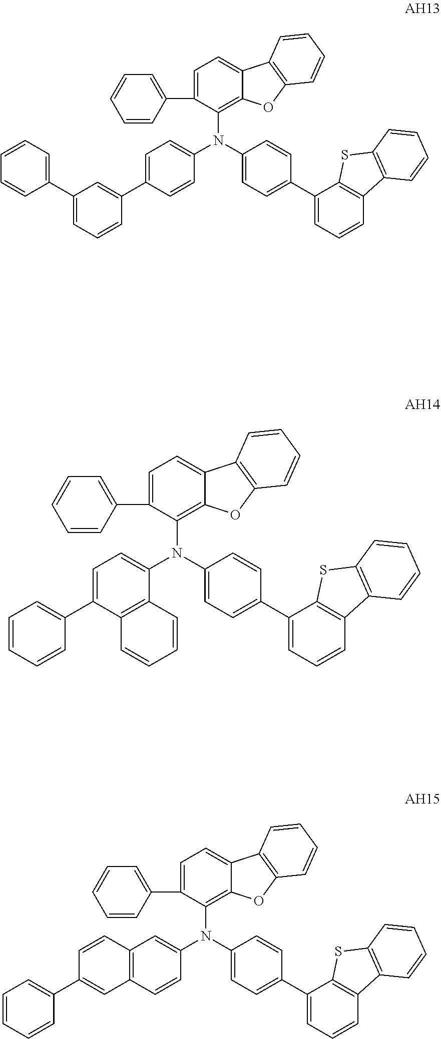 [SMILES: c1ccc(-c2cccc(-c3ccc(N(c4ccc(-c5cccc6c5sc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3cccc4c3sc3ccccc34)cc2)c2ccc(-c3ccccc3)c3ccccc23)cc1 ; c1ccc(-c2ccc3cc(N(c4ccc(-c5cccc6c5sc5ccccc56)cc4)c4c(-c5ccccc5)ccc5c4oc4ccccc45)ccc3c2)cc1] 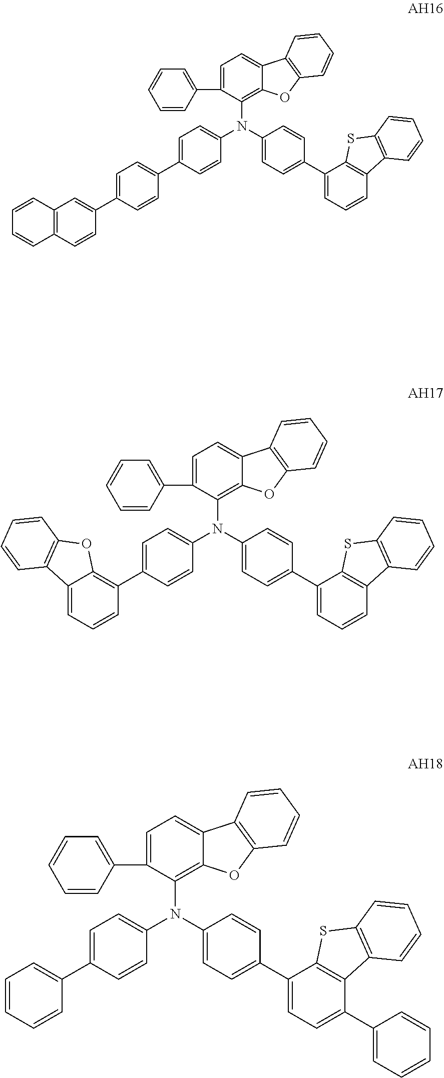 [SMILES: c1ccc(-c2ccc3c(oc4ccccc43)c2N(c2ccc(-c3ccc(-c4ccc5ccccc5c4)cc3)cc2)c2ccc(-c3cccc4c3sc3ccccc34)cc2)cc1 ; c1ccc(-c2ccc3c(oc4ccccc43)c2N(c2ccc(-c3cccc4c3oc3ccccc34)cc2)c2ccc(-c3cccc4c3sc3ccccc34)cc2)cc1 ; c1ccc(-c2ccc(N(c3ccc(-c4ccc(-c5ccccc5)c5c4sc4ccccc45)cc3)c3c(-c4ccccc4)ccc4c3oc3ccccc34)cc2)cc1] 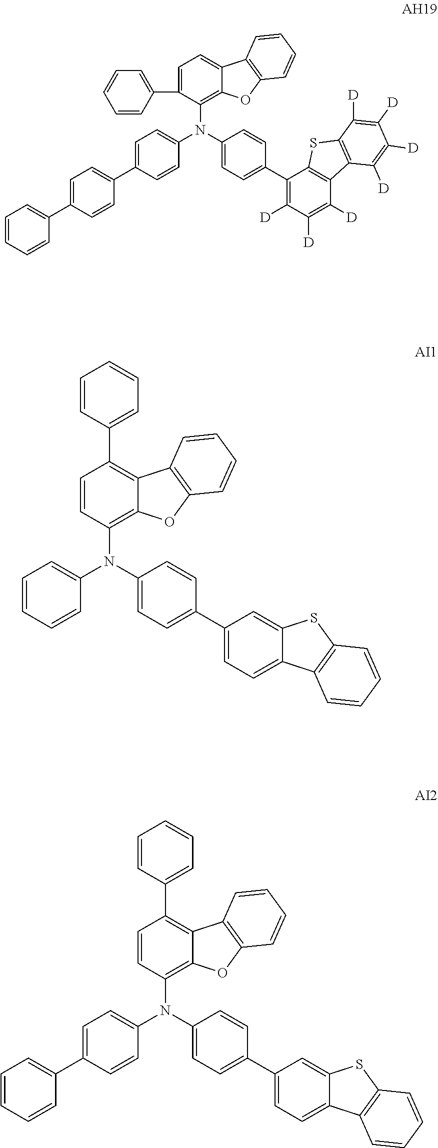 [SMILES: [2H]c1c([2H])c([2H])c2c(sc3c(-c4ccc(N(c5ccc(-c6ccc(-c7ccccc7)cc6)cc5)c5c(-c6ccccc6)ccc6c5oc5ccccc56)cc4)c([2H])c([2H])c([2H])c32)c1[2H] ; c1ccc(-c2ccc(N(c3ccccc3)c3ccc(-c4ccc5c(c4)sc4ccccc45)cc3)c3oc4ccccc4c23)cc1 ; c1ccc(-c2ccc(N(c3ccc(-c4ccc5c(c4)sc4ccccc45)cc3)c3ccc(-c4ccccc4)c4c3oc3ccccc34)cc2)cc1] 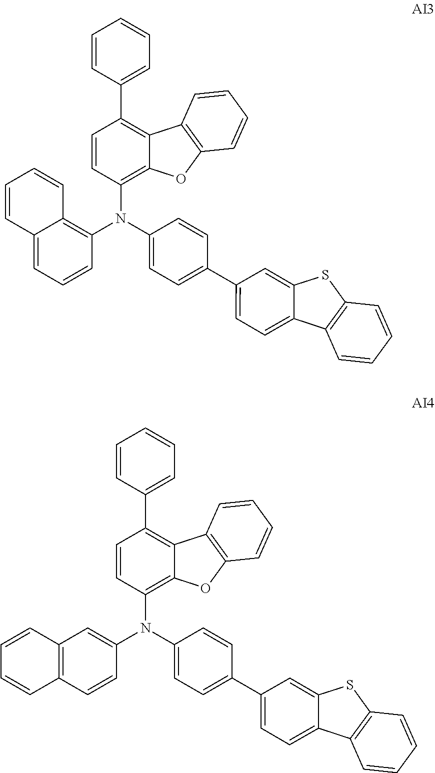 [SMILES: c1ccc(-c2ccc(N(c3ccc(-c4ccc5c(c4)sc4ccccc45)cc3)c3cccc4ccccc34)c3oc4ccccc4c23)cc1 ; c1ccc(-c2ccc(N(c3ccc(-c4ccc5c(c4)sc4ccccc45)cc3)c3ccc4ccccc4c3)c3oc4ccccc4c23)cc1] 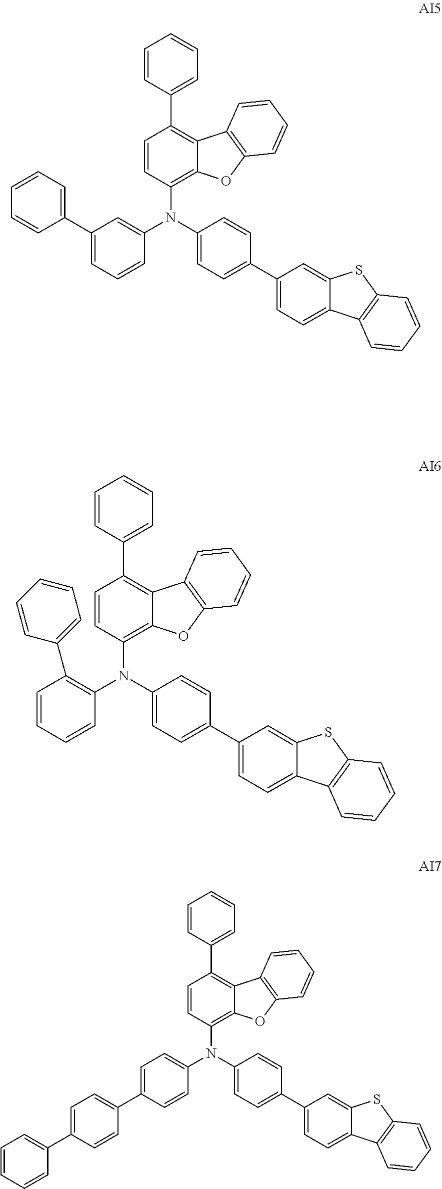 [SMILES: c1ccc(-c2cccc(N(c3ccc(-c4ccc5c(c4)sc4ccccc45)cc3)c3ccc(-c4ccccc4)c4c3oc3ccccc34)c2)cc1 ; c1ccc(-c2ccccc2N(c2ccc(-c3ccc4c(c3)sc3ccccc34)cc2)c2ccc(-c3ccccc3)c3c2oc2ccccc23)cc1 ; c1ccc(-c2ccc(-c3ccc(N(c4ccc(-c5ccc6c(c5)sc5ccccc56)cc4)c4ccc(-c5ccccc5)c5c4oc4ccccc45)cc3)cc2)cc1] 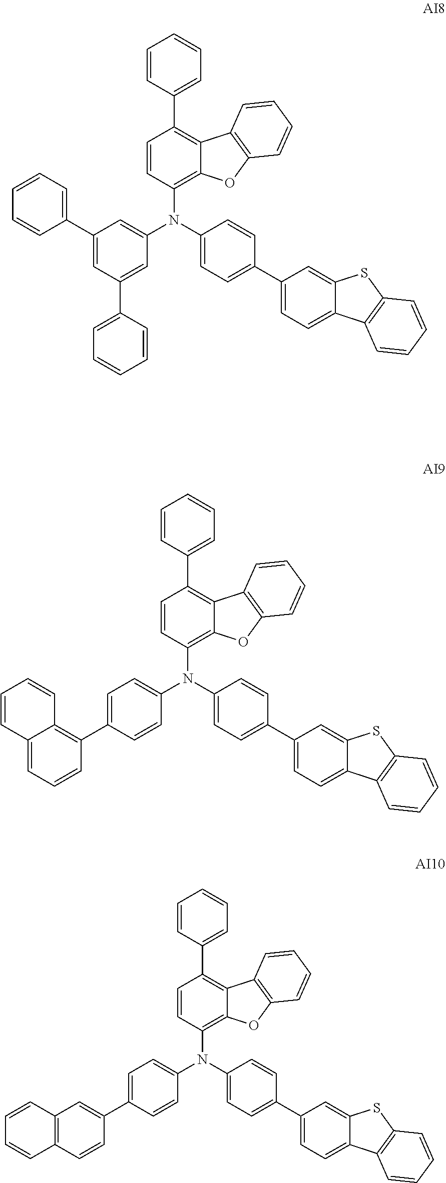 [SMILES: c1ccc(-c2cc(-c3ccccc3)cc(N(c3ccc(-c4ccc5c(c4)sc4ccccc45)cc3)c3ccc(-c4ccccc4)c4c3oc3ccccc34)c2)cc1 ; c1ccc(-c2ccc(N(c3ccc(-c4ccc5c(c4)sc4ccccc45)cc3)c3ccc(-c4cccc5ccccc45)cc3)c3oc4ccccc4c23)cc1 ; c1ccc(-c2ccc(N(c3ccc(-c4ccc5ccccc5c4)cc3)c3ccc(-c4ccc5c(c4)sc4ccccc45)cc3)c3oc4ccccc4c23)cc1] 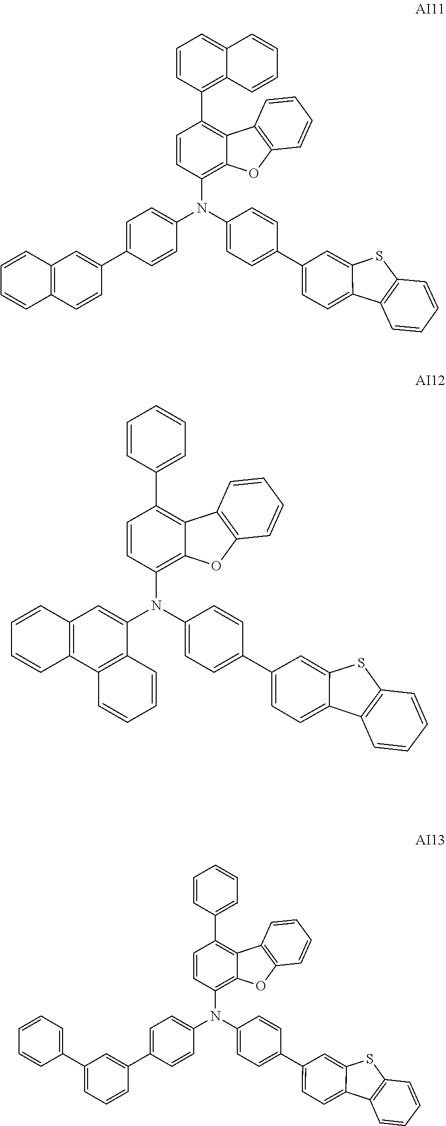 [SMILES: c1ccc2cc(-c3ccc(N(c4ccc(-c5ccc6c(c5)sc5ccccc56)cc4)c4ccc(-c5cccc6ccccc56)c5c4oc4ccccc45)cc3)ccc2c1 ; c1ccc(-c2ccc(N(c3ccc(-c4ccc5c(c4)sc4ccccc45)cc3)c3cc4ccccc4c4ccccc34)c3oc4ccccc4c23)cc1 ; c1ccc(-c2cccc(-c3ccc(N(c4ccc(-c5ccc6c(c5)sc5ccccc56)cc4)c4ccc(-c5ccccc5)c5c4oc4ccccc45)cc3)c2)cc1] 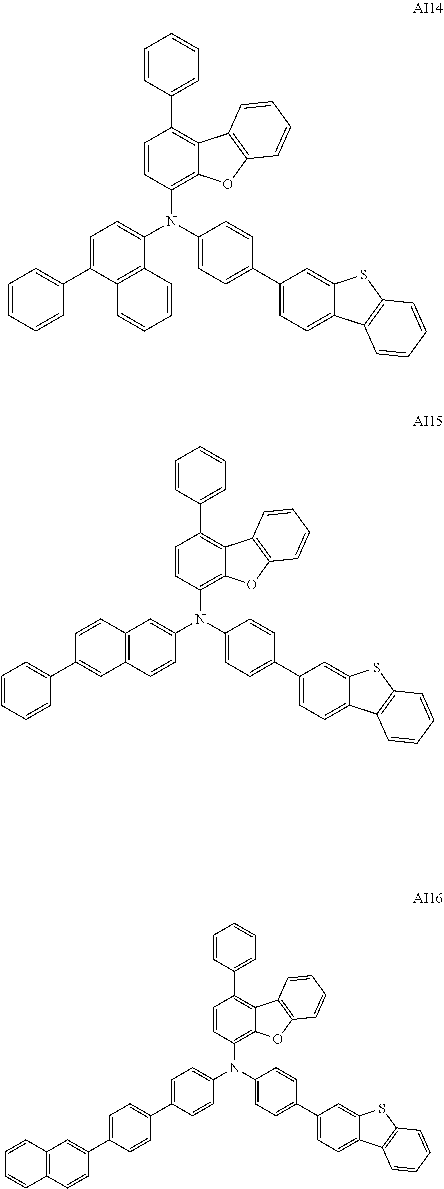 [SMILES: c1ccc(-c2ccc(N(c3ccc(-c4ccc5c(c4)sc4ccccc45)cc3)c3ccc(-c4ccccc4)c4c3oc3ccccc34)c3ccccc23)cc1 ; c1ccc(-c2ccc3cc(N(c4ccc(-c5ccc6c(c5)sc5ccccc56)cc4)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc(-c4ccc5c(c4)sc4ccccc45)cc3)c3oc4ccccc4c23)cc1] 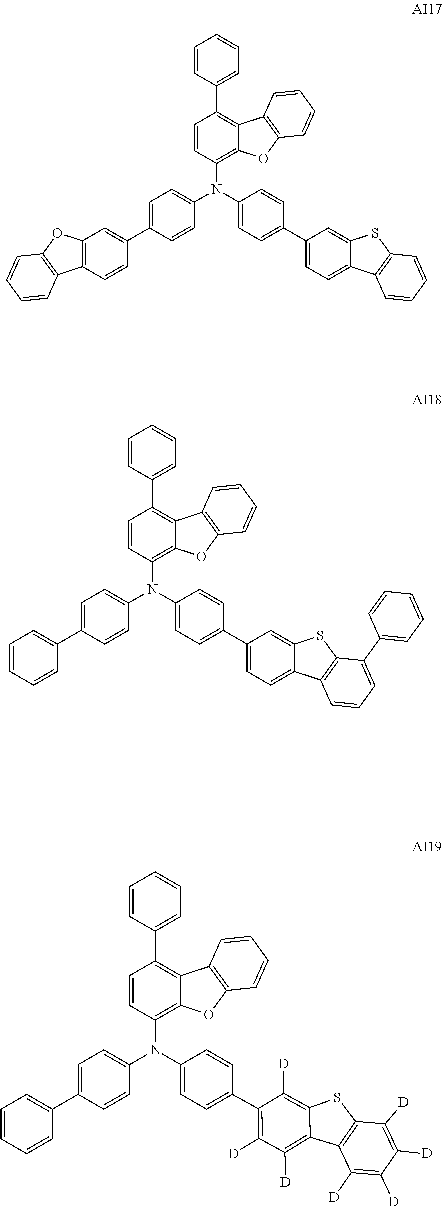 [SMILES: c1ccc(-c2ccc(N(c3ccc(-c4ccc5c(c4)oc4ccccc45)cc3)c3ccc(-c4ccc5c(c4)sc4ccccc45)cc3)c3oc4ccccc4c23)cc1 ; c1ccc(-c2ccc(N(c3ccc(-c4ccc5c(c4)sc4c(-c6ccccc6)cccc45)cc3)c3ccc(-c4ccccc4)c4c3oc3ccccc34)cc2)cc1 ; [2H]c1c([2H])c([2H])c2c(sc3c([2H])c(-c4ccc(N(c5ccc(-c6ccccc6)cc5)c5ccc(-c6ccccc6)c6c5oc5ccccc56)cc4)c([2H])c([2H])c32)c1[2H]] 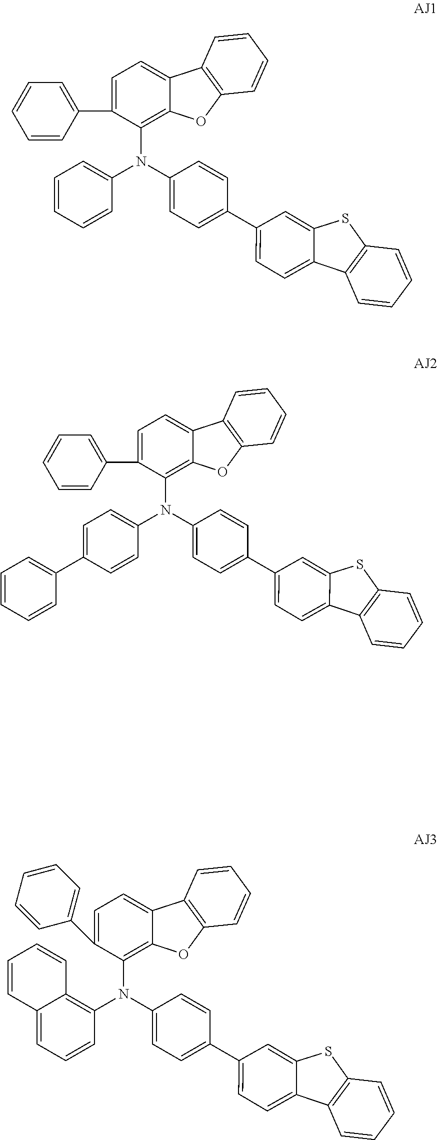 [SMILES: c1ccc(-c2ccc3c(oc4ccccc43)c2N(c2ccccc2)c2ccc(-c3ccc4c(c3)sc3ccccc34)cc2)cc1 ; c1ccc(-c2ccc(N(c3ccc(-c4ccc5c(c4)sc4ccccc45)cc3)c3c(-c4ccccc4)ccc4c3oc3ccccc34)cc2)cc1 ; c1ccc(-c2ccc3c(oc4ccccc43)c2N(c2ccc(-c3ccc4c(c3)sc3ccccc34)cc2)c2cccc3ccccc23)cc1] 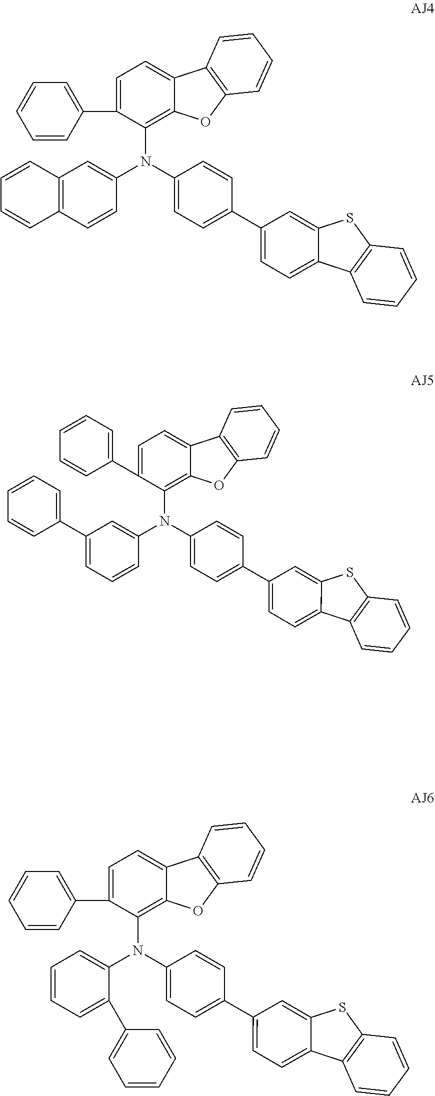 [SMILES: c1ccc(-c2ccc3c(oc4ccccc43)c2N(c2ccc(-c3ccc4c(c3)sc3ccccc34)cc2)c2ccc3ccccc3c2)cc1 ; c1ccc(-c2cccc(N(c3ccc(-c4ccc5c(c4)sc4ccccc45)cc3)c3c(-c4ccccc4)ccc4c3oc3ccccc34)c2)cc1 ; c1ccc(-c2ccccc2N(c2ccc(-c3ccc4c(c3)sc3ccccc34)cc2)c2c(-c3ccccc3)ccc3c2oc2ccccc23)cc1] 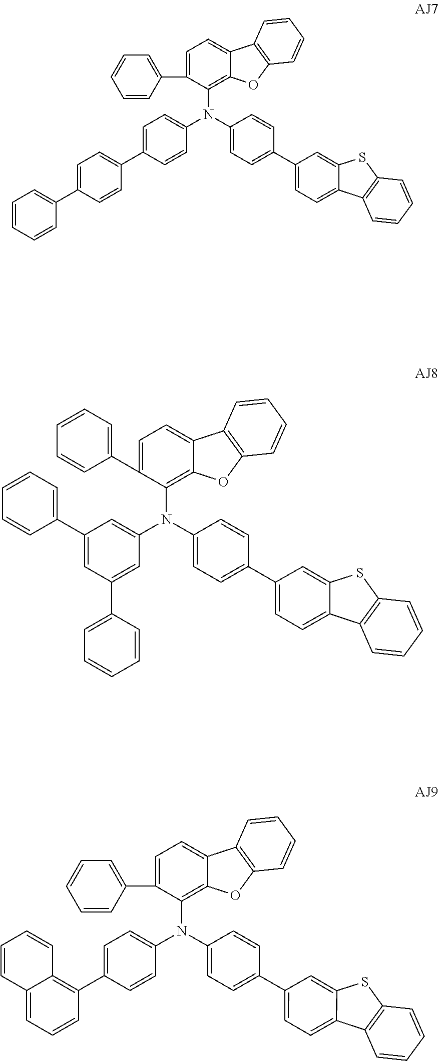 [SMILES: c1ccc(-c2ccc(-c3ccc(N(c4ccc(-c5ccc6c(c5)sc5ccccc56)cc4)c4c(-c5ccccc5)ccc5c4oc4ccccc45)cc3)cc2)cc1 ; c1ccc(-c2cc(-c3ccccc3)cc(N(c3ccc(-c4ccc5c(c4)sc4ccccc45)cc3)c3c(-c4ccccc4)ccc4c3oc3ccccc34)c2)cc1 ; c1ccc(-c2ccc3c(oc4ccccc43)c2N(c2ccc(-c3ccc4c(c3)sc3ccccc34)cc2)c2ccc(-c3cccc4ccccc34)cc2)cc1] 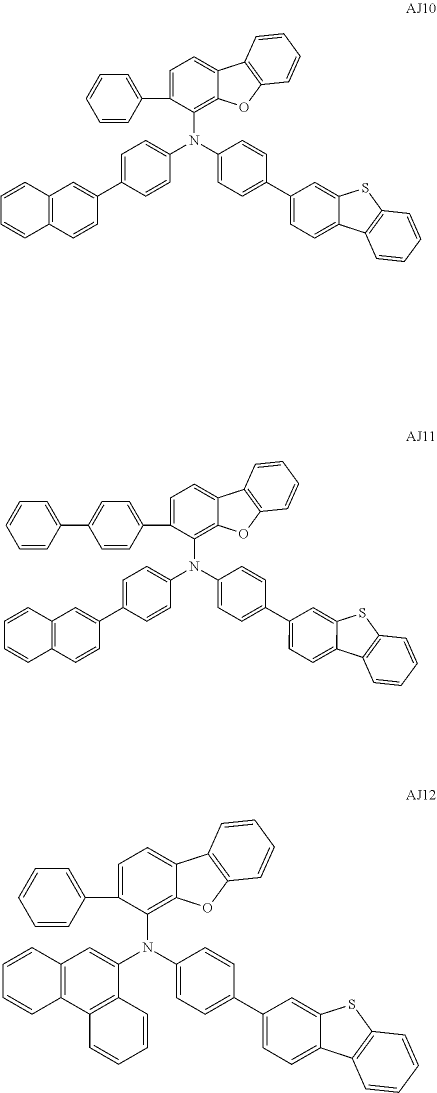 [SMILES: c1ccc(-c2ccc3c(oc4ccccc43)c2N(c2ccc(-c3ccc4ccccc4c3)cc2)c2ccc(-c3ccc4c(c3)sc3ccccc34)cc2)cc1 ; c1ccc(-c2ccc(-c3ccc4c(oc5ccccc54)c3N(c3ccc(-c4ccc5ccccc5c4)cc3)c3ccc(-c4ccc5c(c4)sc4ccccc45)cc3)cc2)cc1 ; c1ccc(-c2ccc3c(oc4ccccc43)c2N(c2ccc(-c3ccc4c(c3)sc3ccccc34)cc2)c2cc3ccccc3c3ccccc23)cc1] 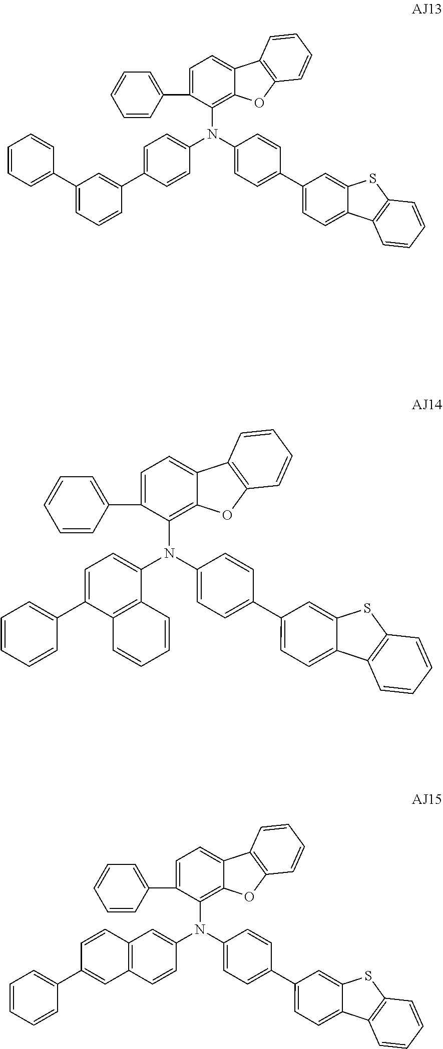 [SMILES: c1ccc(-c2cccc(-c3ccc(N(c4ccc(-c5ccc6c(c5)sc5ccccc56)cc4)c4c(-c5ccccc5)ccc5c4oc4ccccc45)cc3)c2)cc1 ; c1ccc(-c2ccc3c(oc4ccccc43)c2N(c2ccc(-c3ccc4c(c3)sc3ccccc34)cc2)c2ccc(-c3ccccc3)c3ccccc23)cc1 ; c1ccc(-c2ccc3cc(N(c4ccc(-c5ccc6c(c5)sc5ccccc56)cc4)c4c(-c5ccccc5)ccc5c4oc4ccccc45)ccc3c2)cc1] 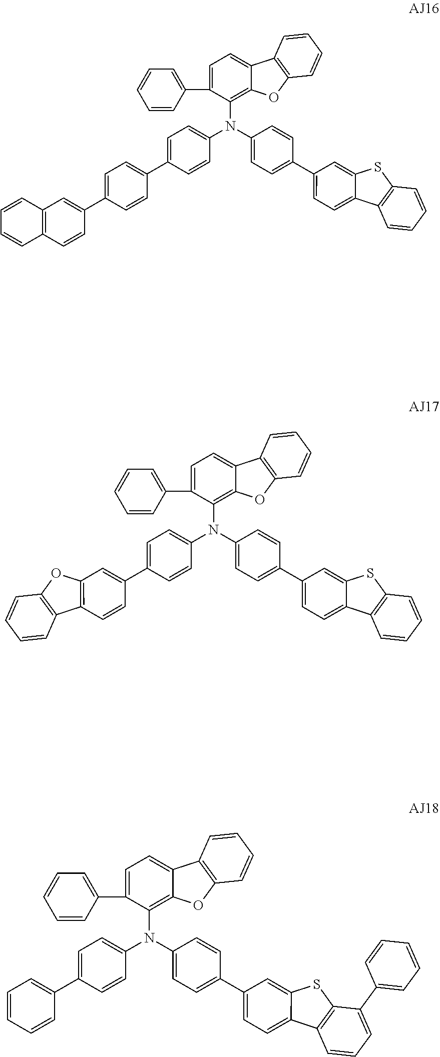 [SMILES: c1ccc(-c2ccc3c(oc4ccccc43)c2N(c2ccc(-c3ccc(-c4ccc5ccccc5c4)cc3)cc2)c2ccc(-c3ccc4c(c3)sc3ccccc34)cc2)cc1 ; c1ccc(-c2ccc3c(oc4ccccc43)c2N(c2ccc(-c3ccc4c(c3)oc3ccccc34)cc2)c2ccc(-c3ccc4c(c3)sc3ccccc34)cc2)cc1 ; c1ccc(-c2ccc(N(c3ccc(-c4ccc5c(c4)sc4c(-c6ccccc6)cccc45)cc3)c3c(-c4ccccc4)ccc4c3oc3ccccc34)cc2)cc1] 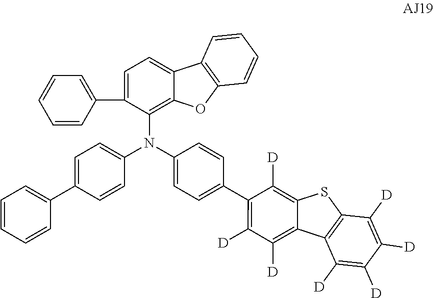 [SMILES: [2H]c1c([2H])c([2H])c2c(sc3c([2H])c(-c4ccc(N(c5ccc(-c6ccccc6)cc5)c5c(-c6ccccc6)ccc6c5oc5ccccc56)cc4)c([2H])c([2H])c32)c1[2H]] 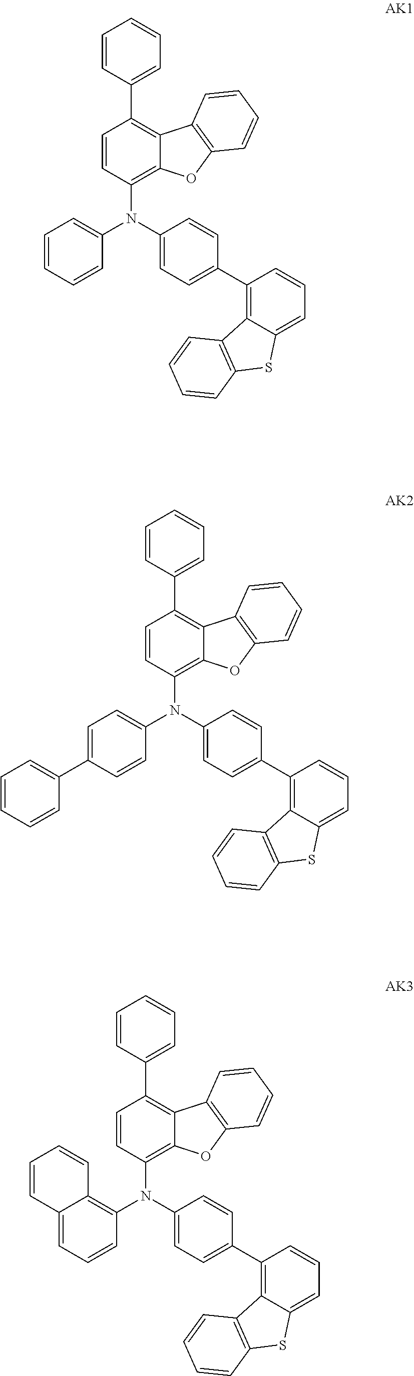 [SMILES: c1ccc(-c2ccc(N(c3ccccc3)c3ccc(-c4cccc5sc6ccccc6c45)cc3)c3oc4ccccc4c23)cc1 ; c1ccc(-c2ccc(N(c3ccc(-c4cccc5sc6ccccc6c45)cc3)c3ccc(-c4ccccc4)c4c3oc3ccccc34)cc2)cc1 ; c1ccc(-c2ccc(N(c3ccc(-c4cccc5sc6ccccc6c45)cc3)c3cccc4ccccc34)c3oc4ccccc4c23)cc1] 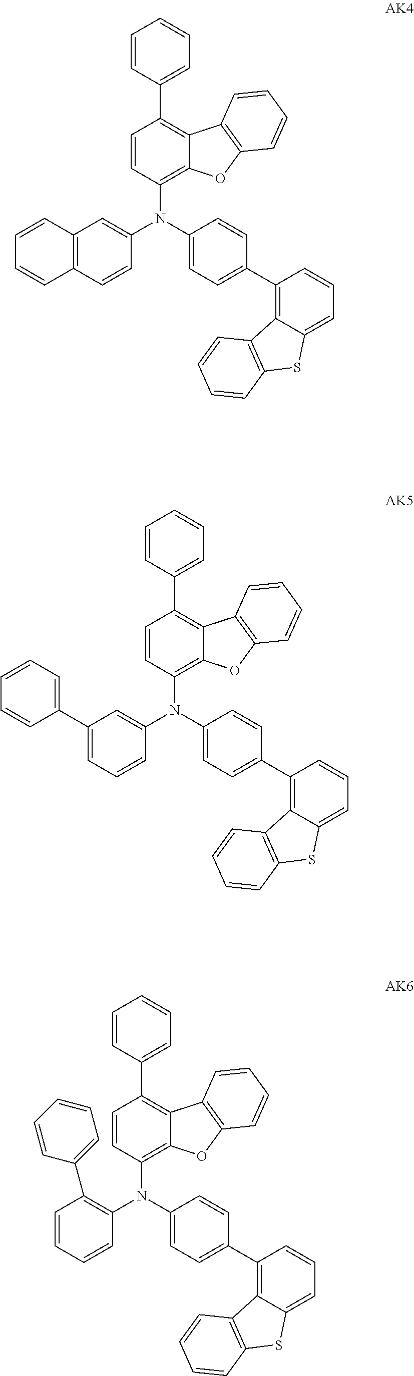 [SMILES: c1ccc(-c2ccc(N(c3ccc(-c4cccc5sc6ccccc6c45)cc3)c3ccc4ccccc4c3)c3oc4ccccc4c23)cc1 ; c1ccc(-c2cccc(N(c3ccc(-c4cccc5sc6ccccc6c45)cc3)c3ccc(-c4ccccc4)c4c3oc3ccccc34)c2)cc1 ; c1ccc(-c2ccccc2N(c2ccc(-c3cccc4sc5ccccc5c34)cc2)c2ccc(-c3ccccc3)c3c2oc2ccccc23)cc1] 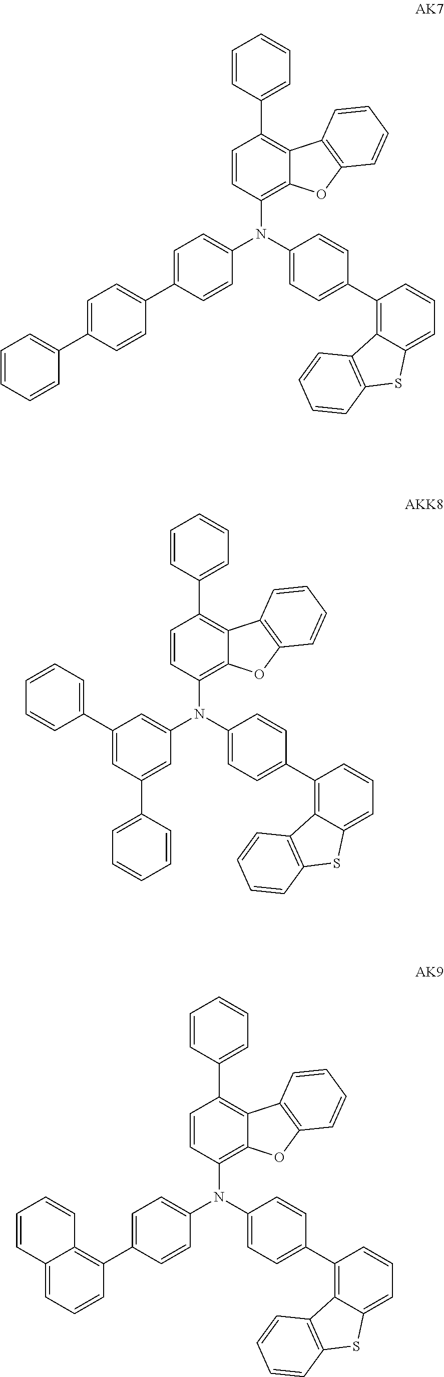 [SMILES: c1ccc(-c2ccc(-c3ccc(N(c4ccc(-c5cccc6sc7ccccc7c56)cc4)c4ccc(-c5ccccc5)c5c4oc4ccccc45)cc3)cc2)cc1 ; c1ccc(-c2cc(-c3ccccc3)cc(N(c3ccc(-c4cccc5sc6ccccc6c45)cc3)c3ccc(-c4ccccc4)c4c3oc3ccccc34)c2)cc1 ; c1ccc(-c2ccc(N(c3ccc(-c4cccc5ccccc45)cc3)c3ccc(-c4cccc5sc6ccccc6c45)cc3)c3oc4ccccc4c23)cc1] 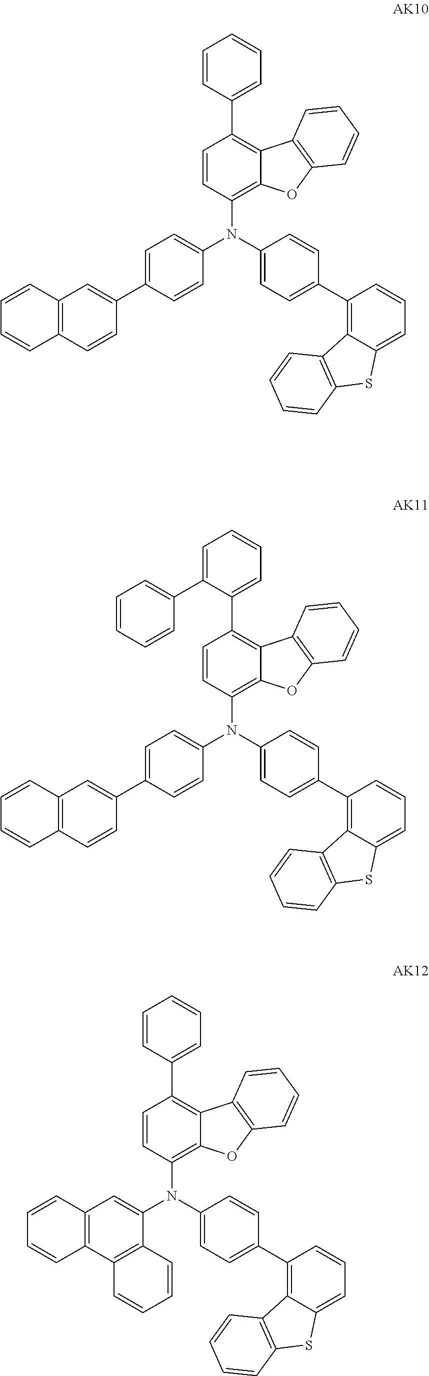 [SMILES: c1ccc(-c2ccc(N(c3ccc(-c4ccc5ccccc5c4)cc3)c3ccc(-c4cccc5sc6ccccc6c45)cc3)c3oc4ccccc4c23)cc1 ; c1ccc(-c2ccccc2-c2ccc(N(c3ccc(-c4ccc5ccccc5c4)cc3)c3ccc(-c4cccc5sc6ccccc6c45)cc3)c3oc4ccccc4c23)cc1 ; c1ccc(-c2ccc(N(c3ccc(-c4cccc5sc6ccccc6c45)cc3)c3cc4ccccc4c4ccccc34)c3oc4ccccc4c23)cc1] 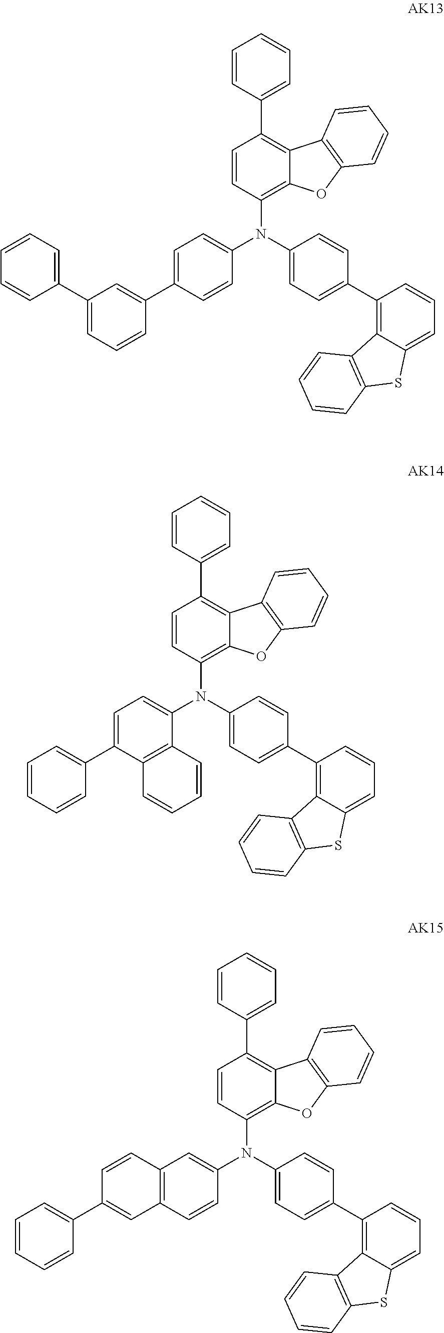 [SMILES: c1ccc(-c2cccc(-c3ccc(N(c4ccc(-c5cccc6sc7ccccc7c56)cc4)c4ccc(-c5ccccc5)c5c4oc4ccccc45)cc3)c2)cc1 ; c1ccc(-c2ccc(N(c3ccc(-c4cccc5sc6ccccc6c45)cc3)c3ccc(-c4ccccc4)c4c3oc3ccccc34)c3ccccc23)cc1 ; c1ccc(-c2ccc3cc(N(c4ccc(-c5cccc6sc7ccccc7c56)cc4)c4ccc(-c5ccccc5)c5c4oc4ccccc45)ccc3c2)cc1] 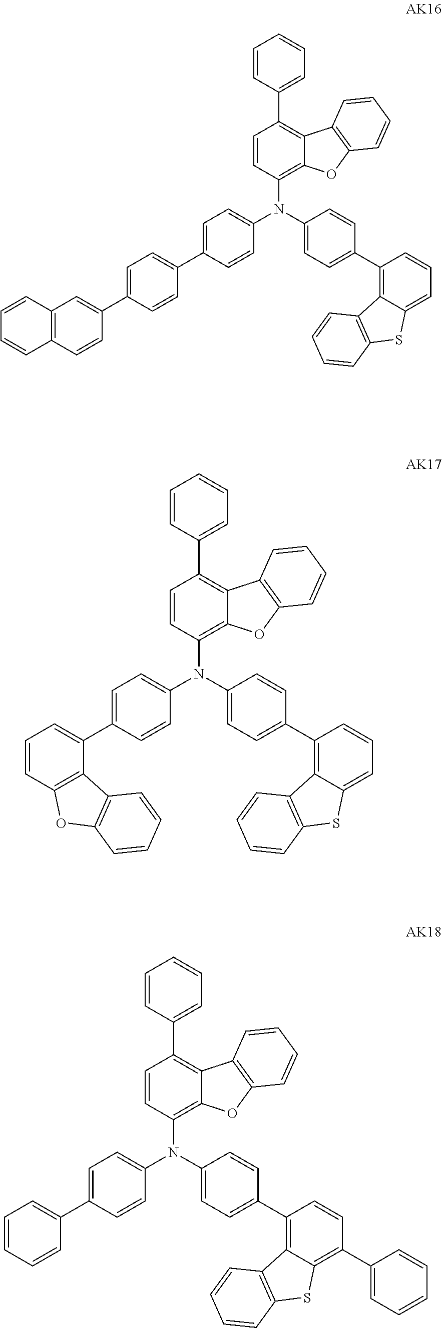 [SMILES: c1ccc(-c2ccc(N(c3ccc(-c4ccc(-c5ccc6ccccc6c5)cc4)cc3)c3ccc(-c4cccc5sc6ccccc6c45)cc3)c3oc4ccccc4c23)cc1 ; c1ccc(-c2ccc(N(c3ccc(-c4cccc5oc6ccccc6c45)cc3)c3ccc(-c4cccc5sc6ccccc6c45)cc3)c3oc4ccccc4c23)cc1 ; c1ccc(-c2ccc(N(c3ccc(-c4ccc(-c5ccccc5)c5sc6ccccc6c45)cc3)c3ccc(-c4ccccc4)c4c3oc3ccccc34)cc2)cc1] 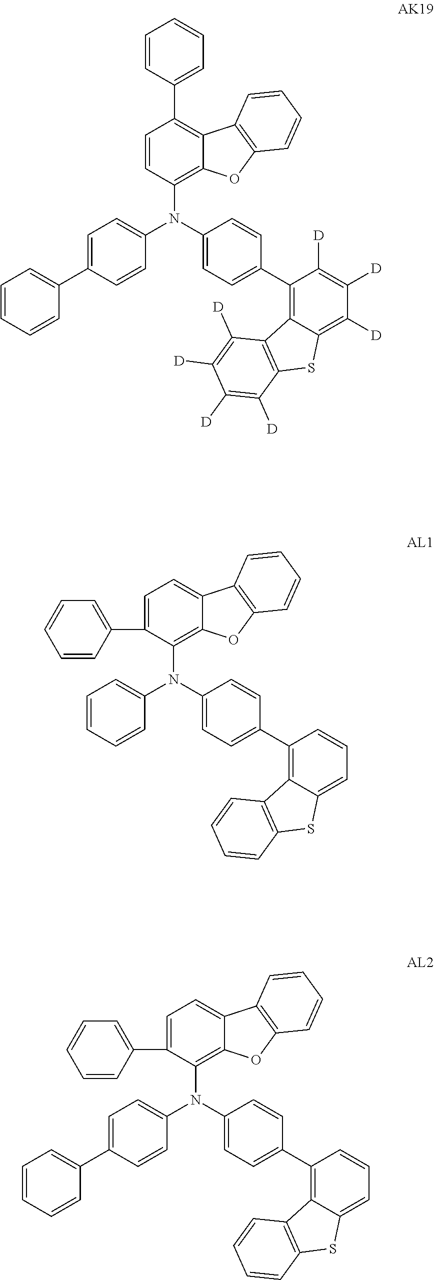 [SMILES: [2H]c1c([2H])c([2H])c2c(sc3c([2H])c([2H])c([2H])c(-c4ccc(N(c5ccc(-c6ccccc6)cc5)c5ccc(-c6ccccc6)c6c5oc5ccccc56)cc4)c32)c1[2H] ; c1ccc(-c2ccc3c(oc4ccccc43)c2N(c2ccccc2)c2ccc(-c3cccc4sc5ccccc5c34)cc2)cc1 ; c1ccc(-c2ccc(N(c3ccc(-c4cccc5sc6ccccc6c45)cc3)c3c(-c4ccccc4)ccc4c3oc3ccccc34)cc2)cc1] 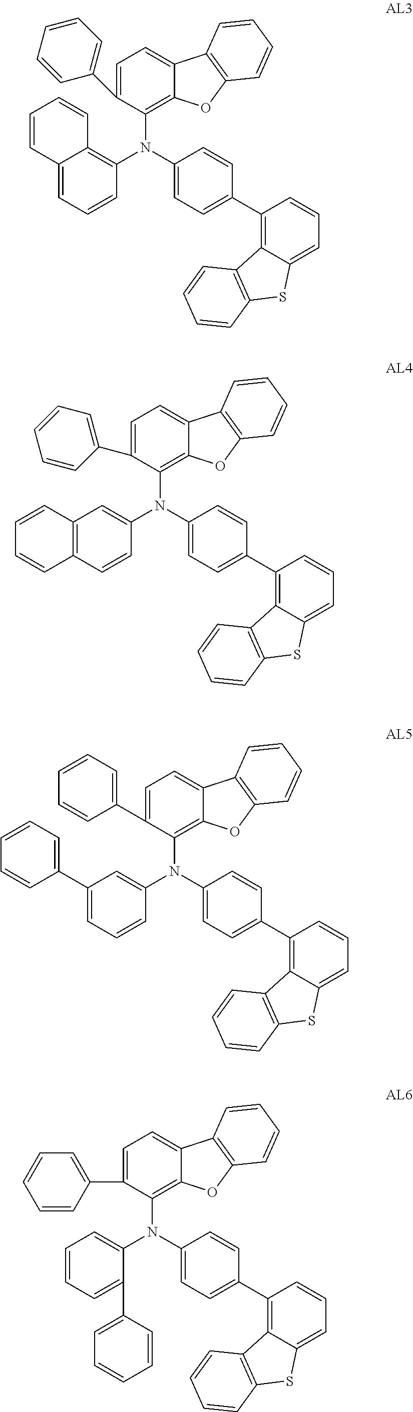 [SMILES: c1ccc(-c2ccc3c(oc4ccccc43)c2N(c2ccc(-c3cccc4sc5ccccc5c34)cc2)c2cccc3ccccc23)cc1 ; c1ccc(-c2ccc3c(oc4ccccc43)c2N(c2ccc(-c3cccc4sc5ccccc5c34)cc2)c2ccc3ccccc3c2)cc1 ; c1ccc(-c2cccc(N(c3ccc(-c4cccc5sc6ccccc6c45)cc3)c3c(-c4ccccc4)ccc4c3oc3ccccc34)c2)cc1 ; c1ccc(-c2ccccc2N(c2ccc(-c3cccc4sc5ccccc5c34)cc2)c2c(-c3ccccc3)ccc3c2oc2ccccc23)cc1] 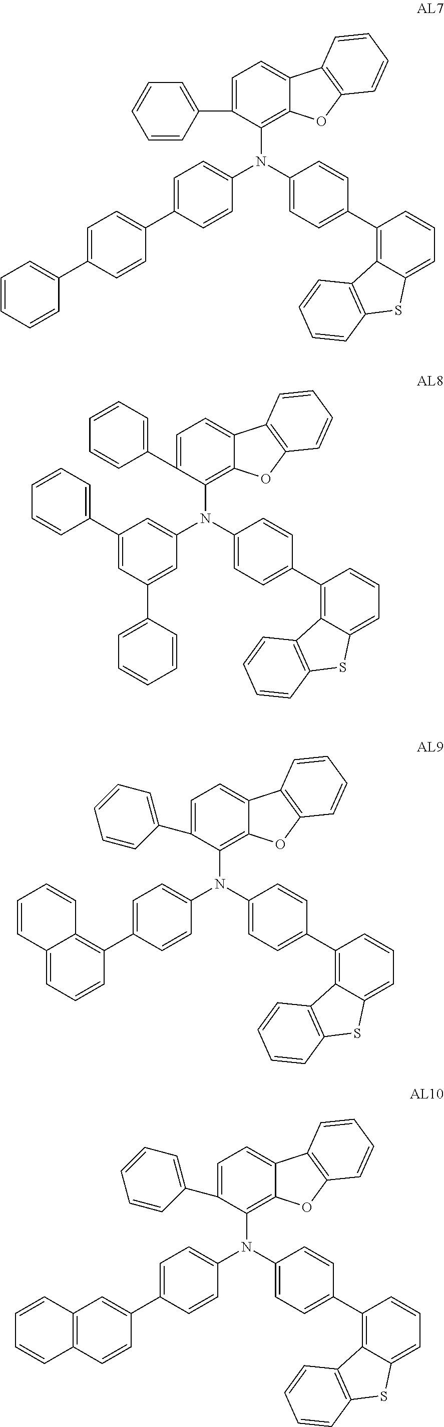 [SMILES: c1ccc(-c2ccc(-c3ccc(N(c4ccc(-c5cccc6sc7ccccc7c56)cc4)c4c(-c5ccccc5)ccc5c4oc4ccccc45)cc3)cc2)cc1 ; c1ccc(-c2cc(-c3ccccc3)cc(N(c3ccc(-c4cccc5sc6ccccc6c45)cc3)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc(-c3cccc4sc5ccccc5c34)cc2)cc1 ; c1ccc(-c2ccc3c(oc4ccccc43)c2N(c2ccc(-c3ccc4ccccc4c3)cc2)c2ccc(-c3cccc4sc5ccccc5c34)cc2)cc1] 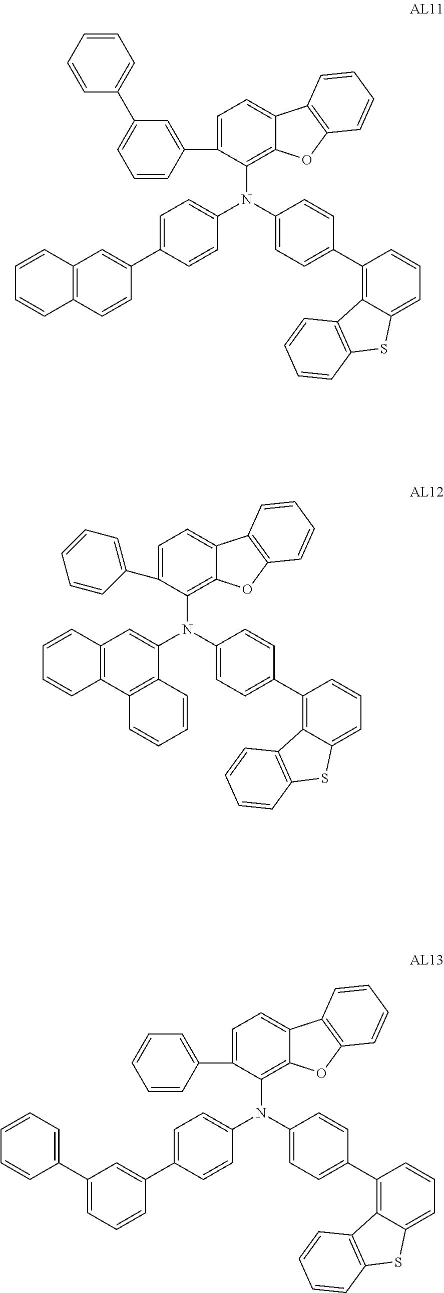 [SMILES: c1ccc(-c2cccc(-c3ccc4c(oc5ccccc54)c3N(c3ccc(-c4ccc5ccccc5c4)cc3)c3ccc(-c4cccc5sc6ccccc6c45)cc3)c2)cc1 ; c1ccc(-c2ccc3c(oc4ccccc43)c2N(c2ccc(-c3cccc4sc5ccccc5c34)cc2)c2cc3ccccc3c3ccccc23)cc1 ; c1ccc(-c2cccc(-c3ccc(N(c4ccc(-c5cccc6sc7ccccc7c56)cc4)c4c(-c5ccccc5)ccc5c4oc4ccccc45)cc3)c2)cc1] 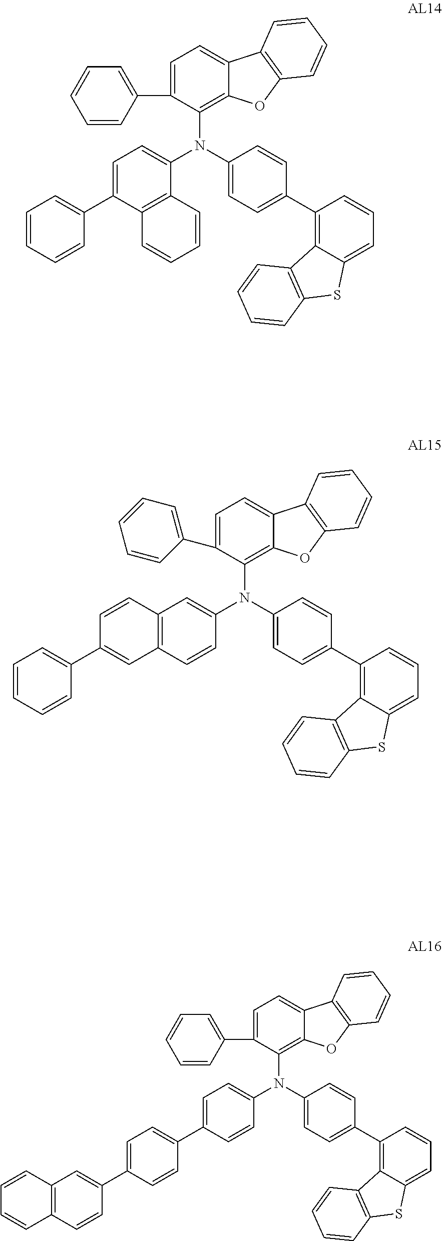 [SMILES: c1ccc(-c2ccc3c(oc4ccccc43)c2N(c2ccc(-c3cccc4sc5ccccc5c34)cc2)c2ccc(-c3ccccc3)c3ccccc23)cc1 ; c1ccc(-c2ccc3cc(N(c4ccc(-c5cccc6sc7ccccc7c56)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3cccc4sc5ccccc5c34)cc2)cc1] 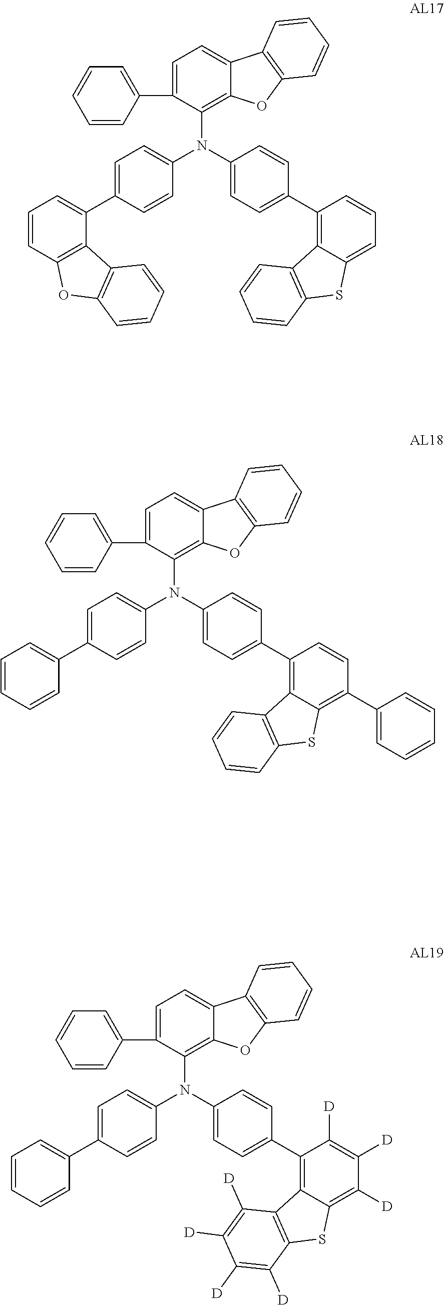 [SMILES: c1ccc(-c2ccc3c(oc4ccccc43)c2N(c2ccc(-c3cccc4oc5ccccc5c34)cc2)c2ccc(-c3cccc4sc5ccccc5c34)cc2)cc1 ; c1ccc(-c2ccc(N(c3ccc(-c4ccc(-c5ccccc5)c5sc6ccccc6c45)cc3)c3c(-c4ccccc4)ccc4c3oc3ccccc34)cc2)cc1 ; [2H]c1c([2H])c([2H])c2c(sc3c([2H])c([2H])c([2H])c(-c4ccc(N(c5ccc(-c6ccccc6)cc5)c5c(-c6ccccc6)ccc6c5oc5ccccc56)cc4)c32)c1[2H]] 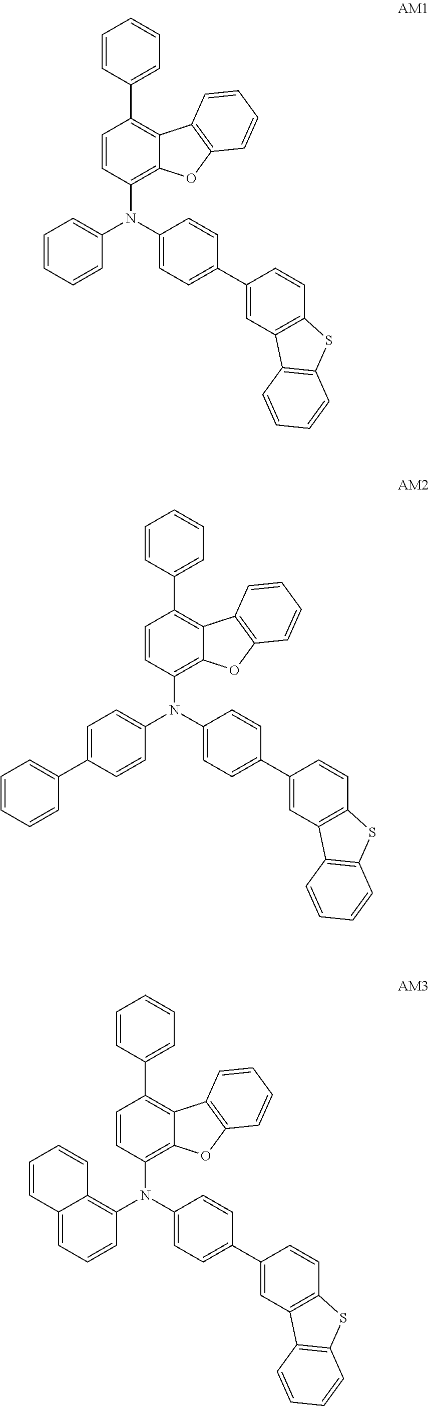 [SMILES: c1ccc(-c2ccc(N(c3ccccc3)c3ccc(-c4ccc5sc6ccccc6c5c4)cc3)c3oc4ccccc4c23)cc1 ; c1ccc(-c2ccc(N(c3ccc(-c4ccc5sc6ccccc6c5c4)cc3)c3ccc(-c4ccccc4)c4c3oc3ccccc34)cc2)cc1 ; c1ccc(-c2ccc(N(c3ccc(-c4ccc5sc6ccccc6c5c4)cc3)c3cccc4ccccc34)c3oc4ccccc4c23)cc1] 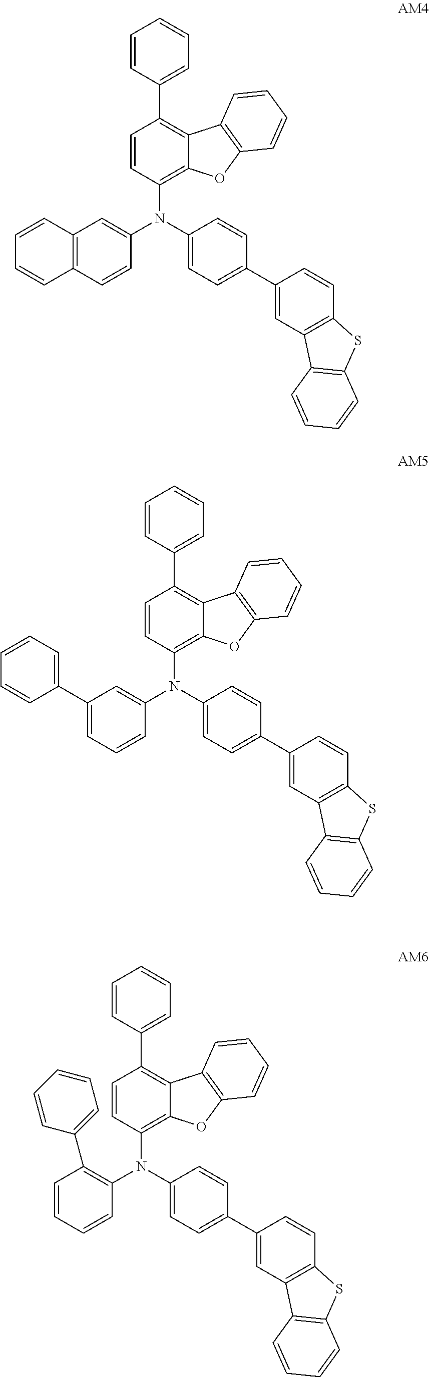 [SMILES: c1ccc(-c2ccc(N(c3ccc(-c4ccc5sc6ccccc6c5c4)cc3)c3ccc4ccccc4c3)c3oc4ccccc4c23)cc1 ; c1ccc(-c2cccc(N(c3ccc(-c4ccc5sc6ccccc6c5c4)cc3)c3ccc(-c4ccccc4)c4c3oc3ccccc34)c2)cc1 ; c1ccc(-c2ccccc2N(c2ccc(-c3ccc4sc5ccccc5c4c3)cc2)c2ccc(-c3ccccc3)c3c2oc2ccccc23)cc1] 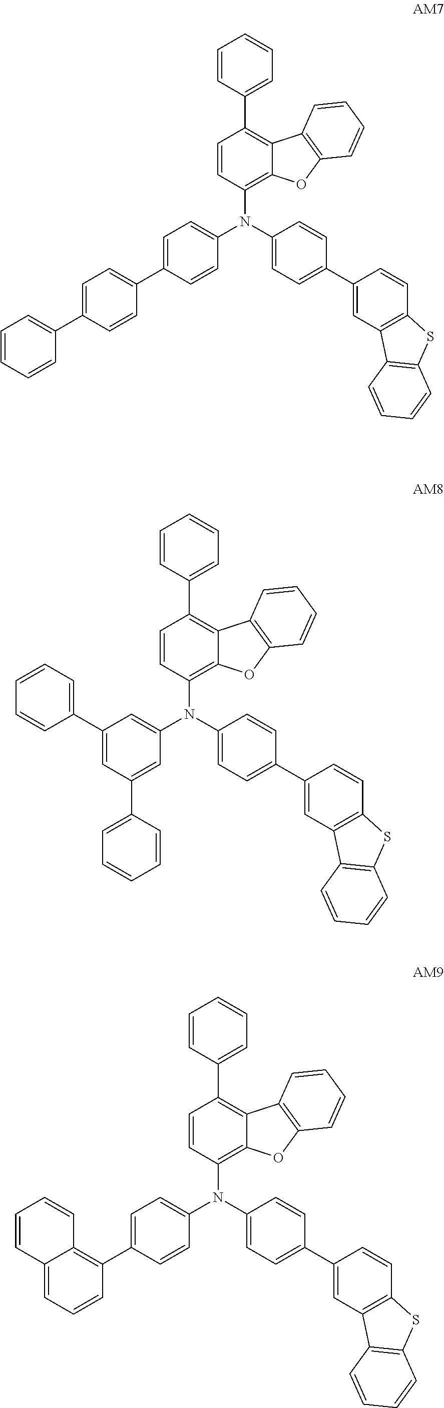 [SMILES: c1ccc(-c2ccc(-c3ccc(N(c4ccc(-c5ccc6sc7ccccc7c6c5)cc4)c4ccc(-c5ccccc5)c5c4oc4ccccc45)cc3)cc2)cc1 ; c1ccc(-c2cc(-c3ccccc3)cc(N(c3ccc(-c4ccc5sc6ccccc6c5c4)cc3)c3ccc(-c4ccccc4)c4c3oc3ccccc34)c2)cc1 ; c1ccc(-c2ccc(N(c3ccc(-c4ccc5sc6ccccc6c5c4)cc3)c3ccc(-c4cccc5ccccc45)cc3)c3oc4ccccc4c23)cc1] 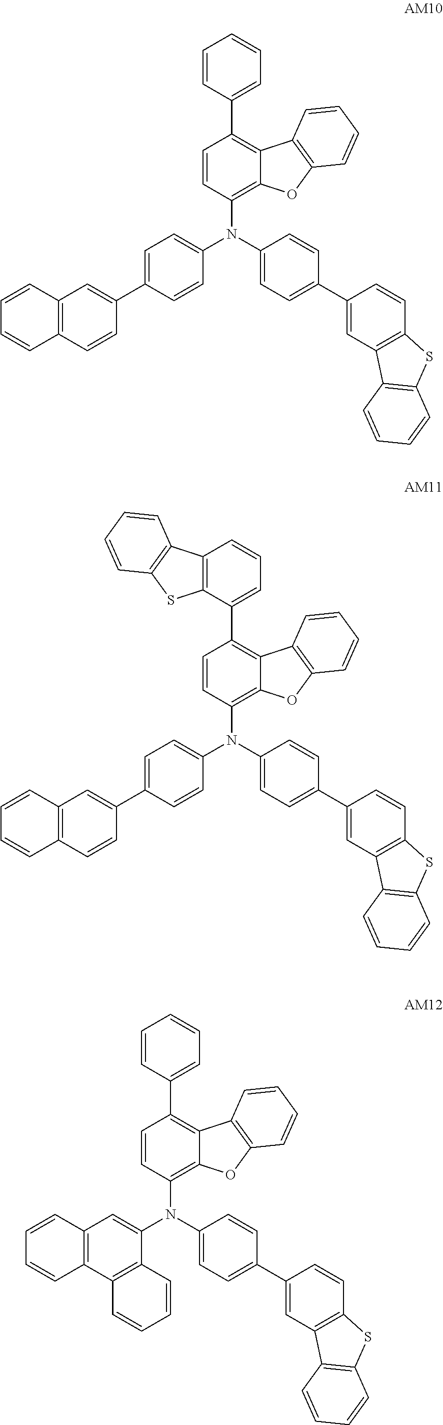 [SMILES: c1ccc(-c2ccc(N(c3ccc(-c4ccc5ccccc5c4)cc3)c3ccc(-c4ccc5sc6ccccc6c5c4)cc3)c3oc4ccccc4c23)cc1 ; c1ccc2cc(-c3ccc(N(c4ccc(-c5ccc6sc7ccccc7c6c5)cc4)c4ccc(-c5cccc6c5sc5ccccc56)c5c4oc4ccccc45)cc3)ccc2c1 ; c1ccc(-c2ccc(N(c3ccc(-c4ccc5sc6ccccc6c5c4)cc3)c3cc4ccccc4c4ccccc34)c3oc4ccccc4c23)cc1] 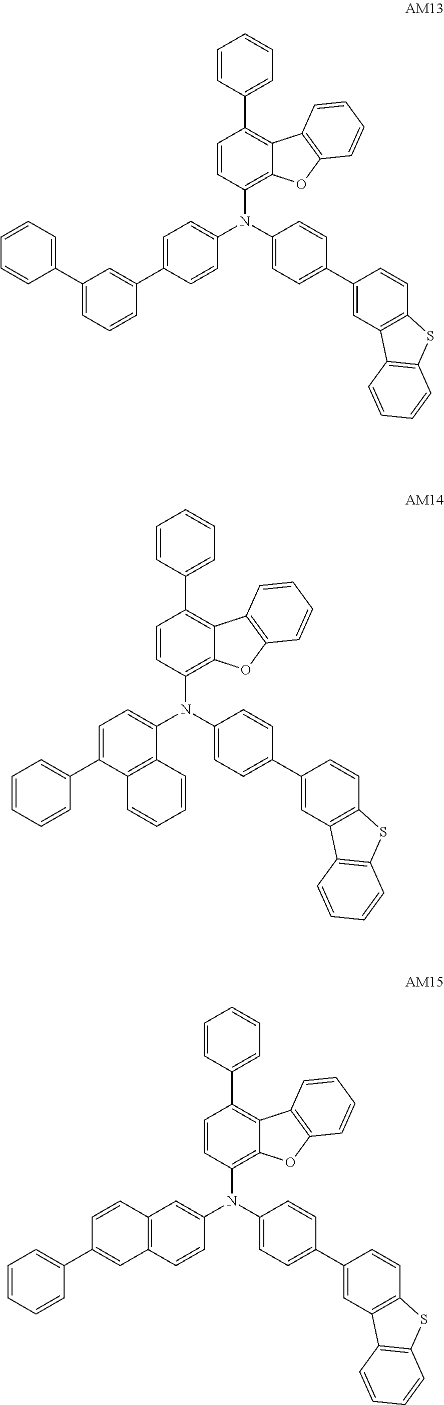 [SMILES: c1ccc(-c2cccc(-c3ccc(N(c4ccc(-c5ccc6sc7ccccc7c6c5)cc4)c4ccc(-c5ccccc5)c5c4oc4ccccc45)cc3)c2)cc1 ; c1ccc(-c2ccc(N(c3ccc(-c4ccc5sc6ccccc6c5c4)cc3)c3ccc(-c4ccccc4)c4c3oc3ccccc34)c3ccccc23)cc1 ; c1ccc(-c2ccc3cc(N(c4ccc(-c5ccc6sc7ccccc7c6c5)cc4)c4ccc(-c5ccccc5)c5c4oc4ccccc45)ccc3c2)cc1] 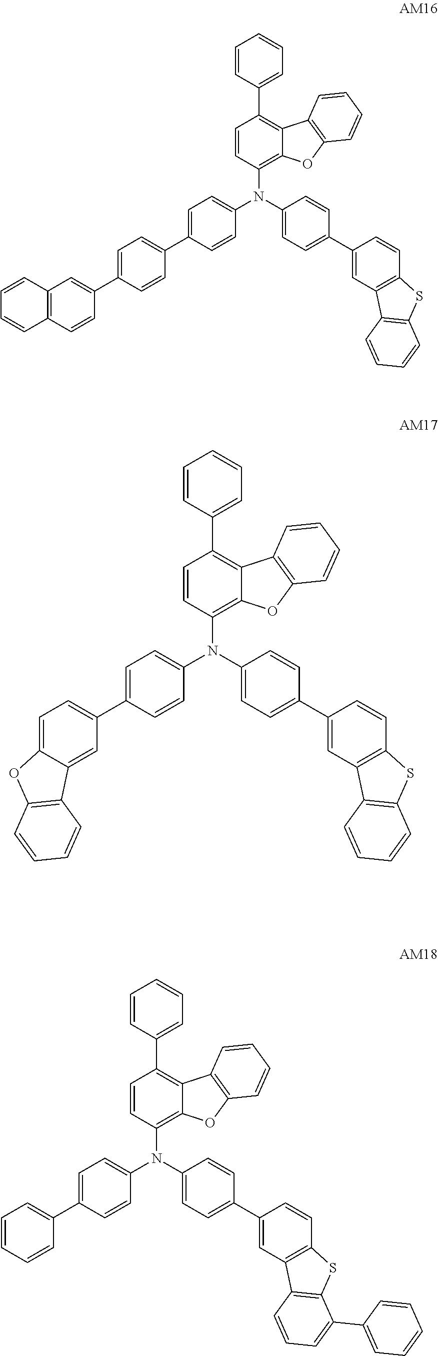 [SMILES: c1ccc(-c2ccc(N(c3ccc(-c4ccc(-c5ccc6ccccc6c5)cc4)cc3)c3ccc(-c4ccc5sc6ccccc6c5c4)cc3)c3oc4ccccc4c23)cc1 ; c1ccc(-c2ccc(N(c3ccc(-c4ccc5oc6ccccc6c5c4)cc3)c3ccc(-c4ccc5sc6ccccc6c5c4)cc3)c3oc4ccccc4c23)cc1 ; c1ccc(-c2ccc(N(c3ccc(-c4ccc5sc6c(-c7ccccc7)cccc6c5c4)cc3)c3ccc(-c4ccccc4)c4c3oc3ccccc34)cc2)cc1] 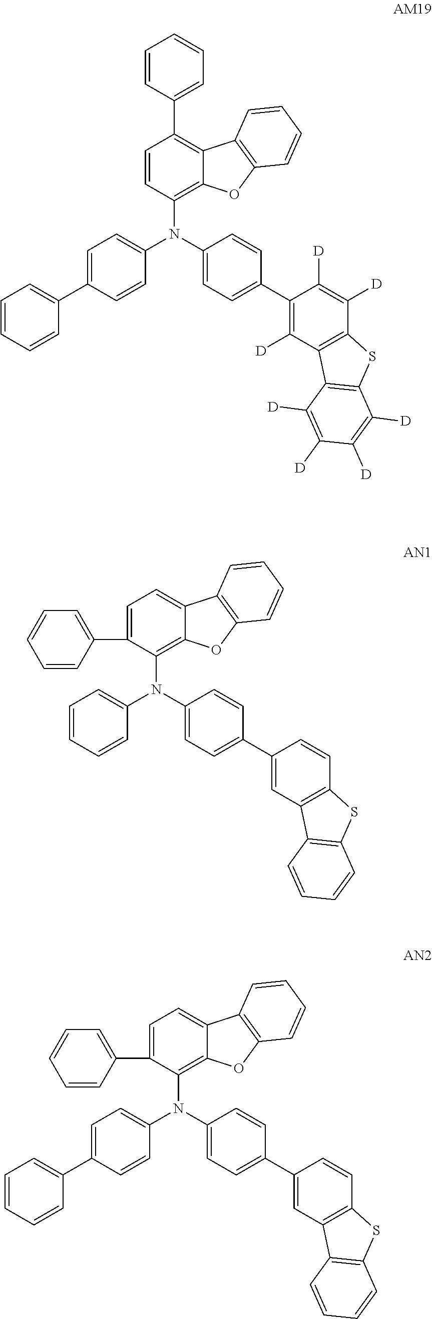 [SMILES: [2H]c1c([2H])c([2H])c2c(sc3c([2H])c([2H])c(-c4ccc(N(c5ccc(-c6ccccc6)cc5)c5ccc(-c6ccccc6)c6c5oc5ccccc56)cc4)c([2H])c32)c1[2H] ; c1ccc(-c2ccc3c(oc4ccccc43)c2N(c2ccccc2)c2ccc(-c3ccc4sc5ccccc5c4c3)cc2)cc1 ; c1ccc(-c2ccc(N(c3ccc(-c4ccc5sc6ccccc6c5c4)cc3)c3c(-c4ccccc4)ccc4c3oc3ccccc34)cc2)cc1] 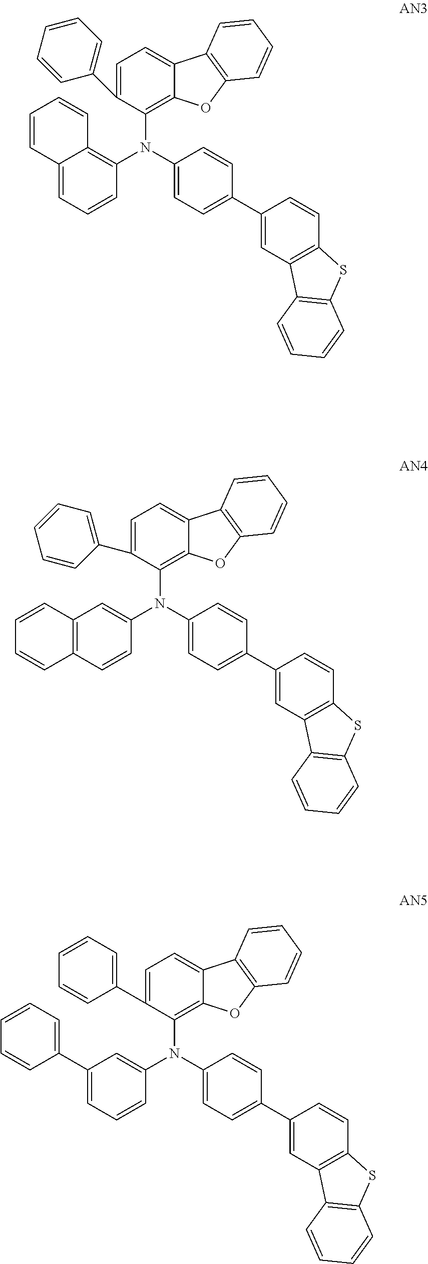 [SMILES: c1ccc(-c2ccc3c(oc4ccccc43)c2N(c2ccc(-c3ccc4sc5ccccc5c4c3)cc2)c2cccc3ccccc23)cc1 ; c1ccc(-c2ccc3c(oc4ccccc43)c2N(c2ccc(-c3ccc4sc5ccccc5c4c3)cc2)c2ccc3ccccc3c2)cc1 ; c1ccc(-c2cccc(N(c3ccc(-c4ccc5sc6ccccc6c5c4)cc3)c3c(-c4ccccc4)ccc4c3oc3ccccc34)c2)cc1] 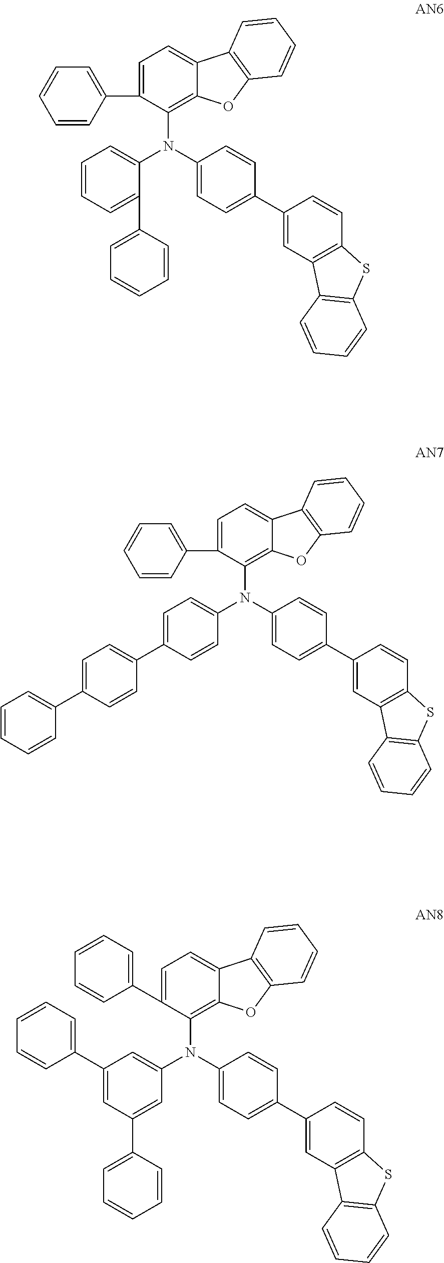 [SMILES: c1ccc(-c2ccccc2N(c2ccc(-c3ccc4sc5ccccc5c4c3)cc2)c2c(-c3ccccc3)ccc3c2oc2ccccc23)cc1 ; c1ccc(-c2ccc(-c3ccc(N(c4ccc(-c5ccc6sc7ccccc7c6c5)cc4)c4c(-c5ccccc5)ccc5c4oc4ccccc45)cc3)cc2)cc1 ; c1ccc(-c2cc(-c3ccccc3)cc(N(c3ccc(-c4ccc5sc6ccccc6c5c4)cc3)c3c(-c4ccccc4)ccc4c3oc3ccccc34)c2)cc1] 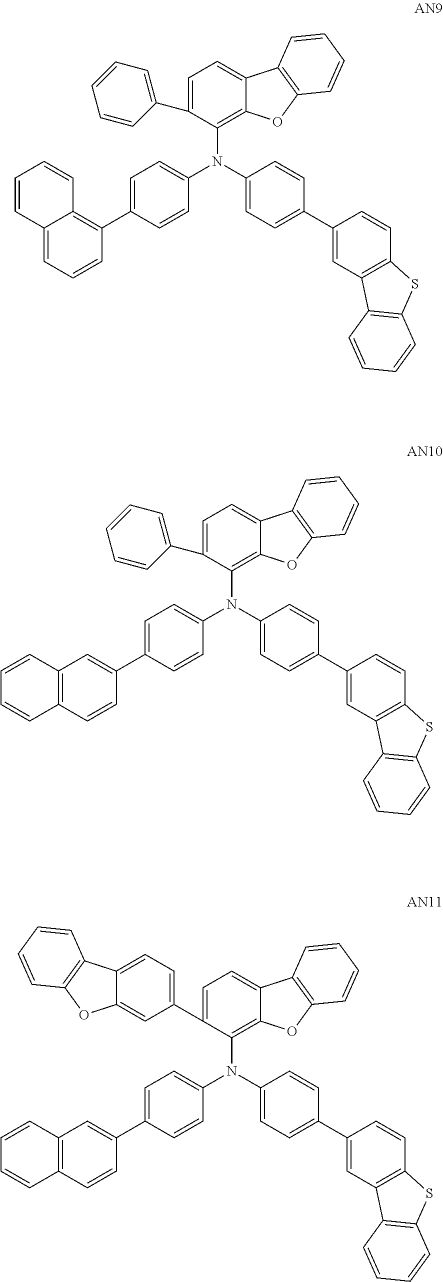 [SMILES: c1ccc(-c2ccc3c(oc4ccccc43)c2N(c2ccc(-c3ccc4sc5ccccc5c4c3)cc2)c2ccc(-c3cccc4ccccc34)cc2)cc1 ; c1ccc(-c2ccc3c(oc4ccccc43)c2N(c2ccc(-c3ccc4ccccc4c3)cc2)c2ccc(-c3ccc4sc5ccccc5c4c3)cc2)cc1 ; c1ccc2cc(-c3ccc(N(c4ccc(-c5ccc6sc7ccccc7c6c5)cc4)c4c(-c5ccc6c(c5)oc5ccccc56)ccc5c4oc4ccccc45)cc3)ccc2c1] 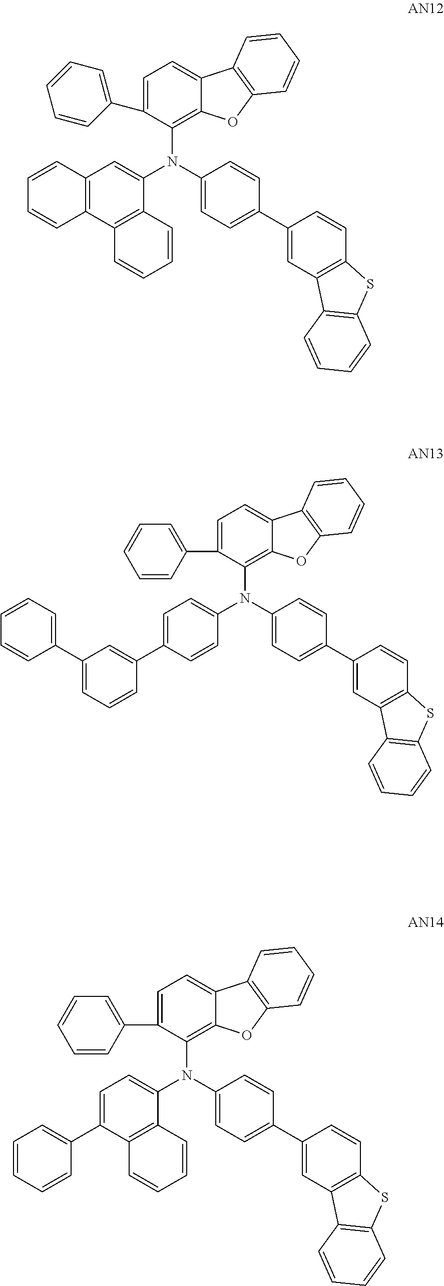 [SMILES: c1ccc(-c2ccc3c(oc4ccccc43)c2N(c2ccc(-c3ccc4sc5ccccc5c4c3)cc2)c2cc3ccccc3c3ccccc23)cc1 ; c1ccc(-c2cccc(-c3ccc(N(c4ccc(-c5ccc6sc7ccccc7c6c5)cc4)c4c(-c5ccccc5)ccc5c4oc4ccccc45)cc3)c2)cc1 ; c1ccc(-c2ccc3c(oc4ccccc43)c2N(c2ccc(-c3ccc4sc5ccccc5c4c3)cc2)c2ccc(-c3ccccc3)c3ccccc23)cc1] 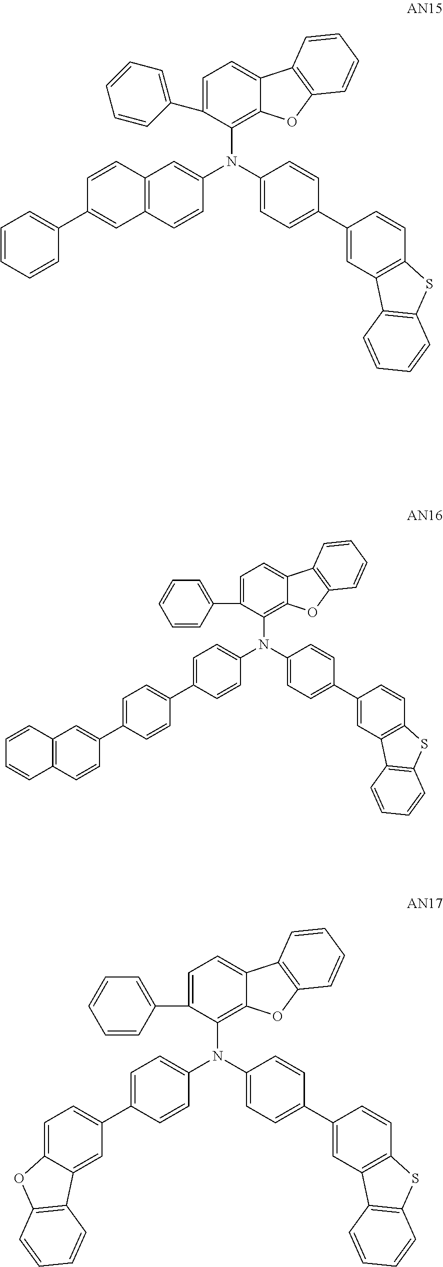 [SMILES: c1ccc(-c2ccc3cc(N(c4ccc(-c5ccc6sc7ccccc7c6c5)cc4)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc(-c3ccc4sc5ccccc5c4c3)cc2)cc1 ; c1ccc(-c2ccc3c(oc4ccccc43)c2N(c2ccc(-c3ccc4oc5ccccc5c4c3)cc2)c2ccc(-c3ccc4sc5ccccc5c4c3)cc2)cc1] 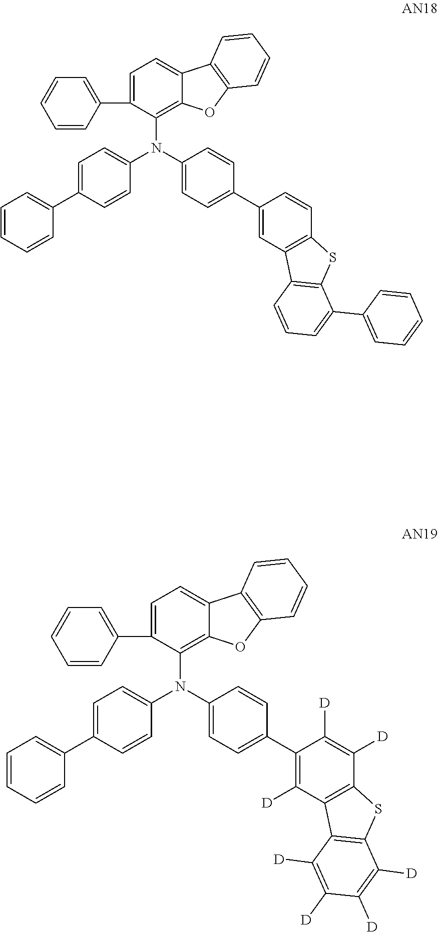 [SMILES: c1ccc(-c2ccc(N(c3ccc(-c4ccc5sc6c(-c7ccccc7)cccc6c5c4)cc3)c3c(-c4ccccc4)ccc4c3oc3ccccc34)cc2)cc1 ; [2H]c1c([2H])c([2H])c2c(sc3c([2H])c([2H])c(-c4ccc(N(c5ccc(-c6ccccc6)cc5)c5c(-c6ccccc6)ccc6c5oc5ccccc56)cc4)c([2H])c32)c1[2H]] 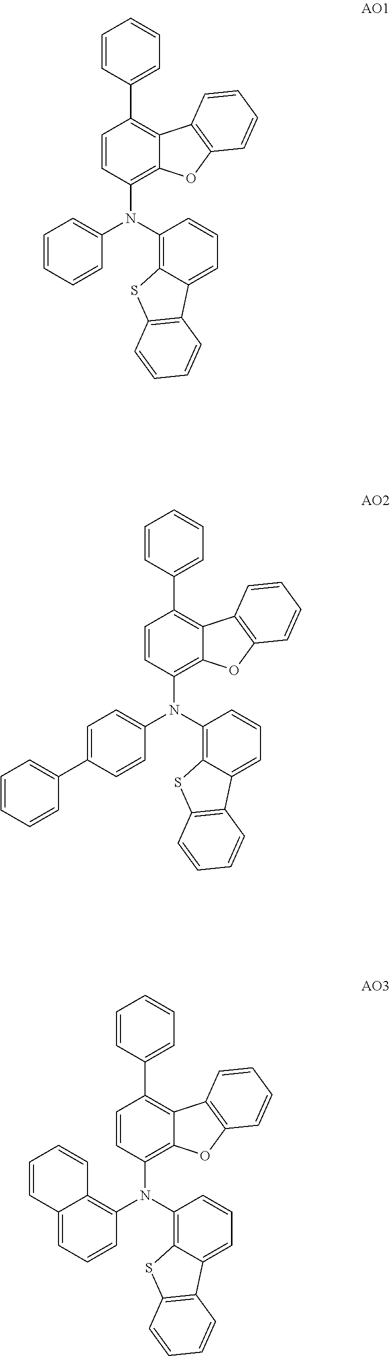 [SMILES: c1ccc(-c2ccc(N(c3ccccc3)c3cccc4c3sc3ccccc34)c3oc4ccccc4c23)cc1 ; c1ccc(-c2ccc(N(c3ccc(-c4ccccc4)c4c3oc3ccccc34)c3cccc4c3sc3ccccc34)cc2)cc1 ; c1ccc(-c2ccc(N(c3cccc4ccccc34)c3cccc4c3sc3ccccc34)c3oc4ccccc4c23)cc1] 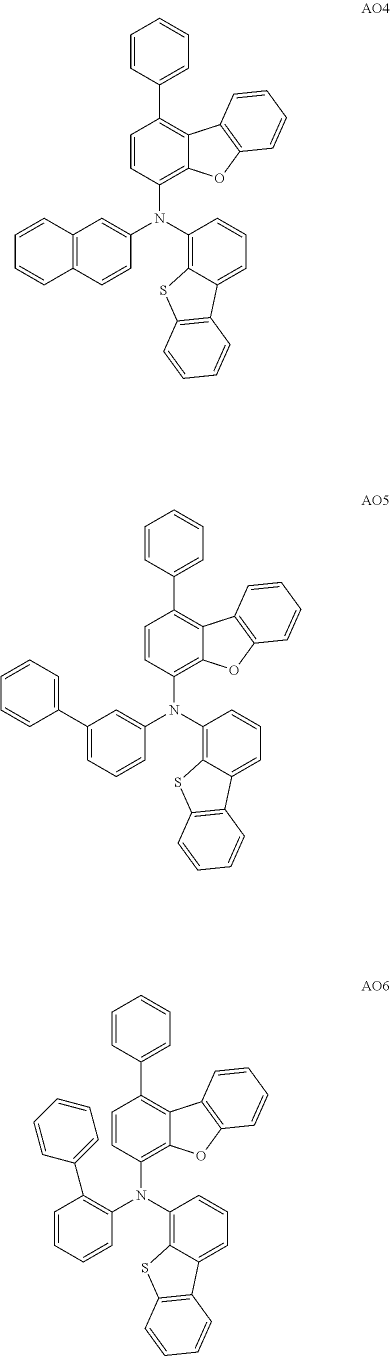 [SMILES: c1ccc(-c2ccc(N(c3ccc4ccccc4c3)c3cccc4c3sc3ccccc34)c3oc4ccccc4c23)cc1 ; c1ccc(-c2cccc(N(c3ccc(-c4ccccc4)c4c3oc3ccccc34)c3cccc4c3sc3ccccc34)c2)cc1 ; c1ccc(-c2ccccc2N(c2ccc(-c3ccccc3)c3c2oc2ccccc23)c2cccc3c2sc2ccccc23)cc1] 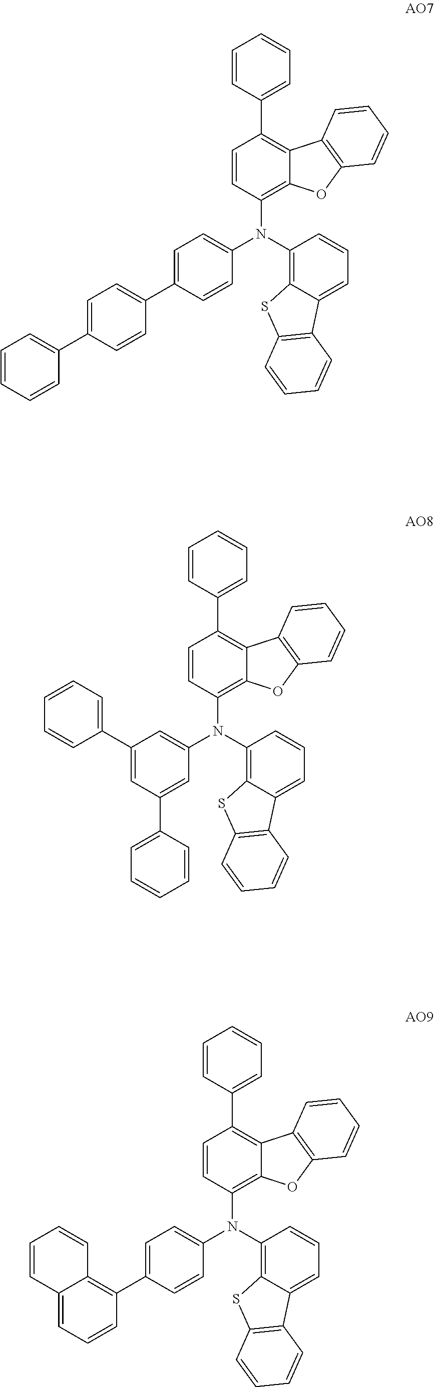 [SMILES: c1ccc(-c2ccc(-c3ccc(N(c4ccc(-c5ccccc5)c5c4oc4ccccc45)c4cccc5c4sc4ccccc45)cc3)cc2)cc1 ; c1ccc(-c2cc(-c3ccccc3)cc(N(c3ccc(-c4ccccc4)c4c3oc3ccccc34)c3cccc4c3sc3ccccc34)c2)cc1 ; c1ccc(-c2ccc(N(c3ccc(-c4cccc5ccccc45)cc3)c3cccc4c3sc3ccccc34)c3oc4ccccc4c23)cc1] 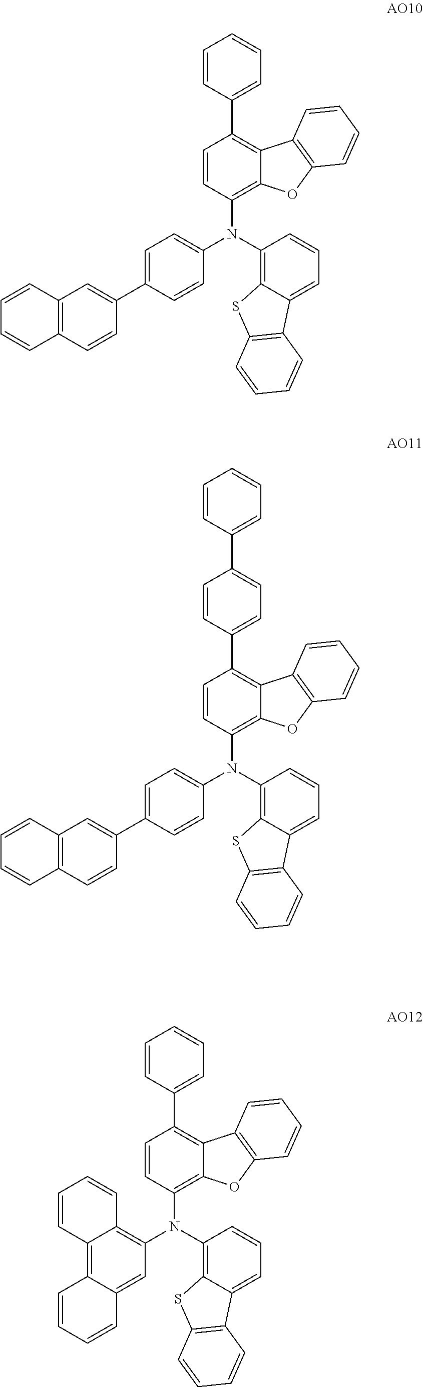 [SMILES: c1ccc(-c2ccc(N(c3ccc(-c4ccc5ccccc5c4)cc3)c3cccc4c3sc3ccccc34)c3oc4ccccc4c23)cc1 ; c1ccc(-c2ccc(-c3ccc(N(c4ccc(-c5ccc6ccccc6c5)cc4)c4cccc5c4sc4ccccc45)c4oc5ccccc5c34)cc2)cc1 ; c1ccc(-c2ccc(N(c3cc4ccccc4c4ccccc34)c3cccc4c3sc3ccccc34)c3oc4ccccc4c23)cc1] 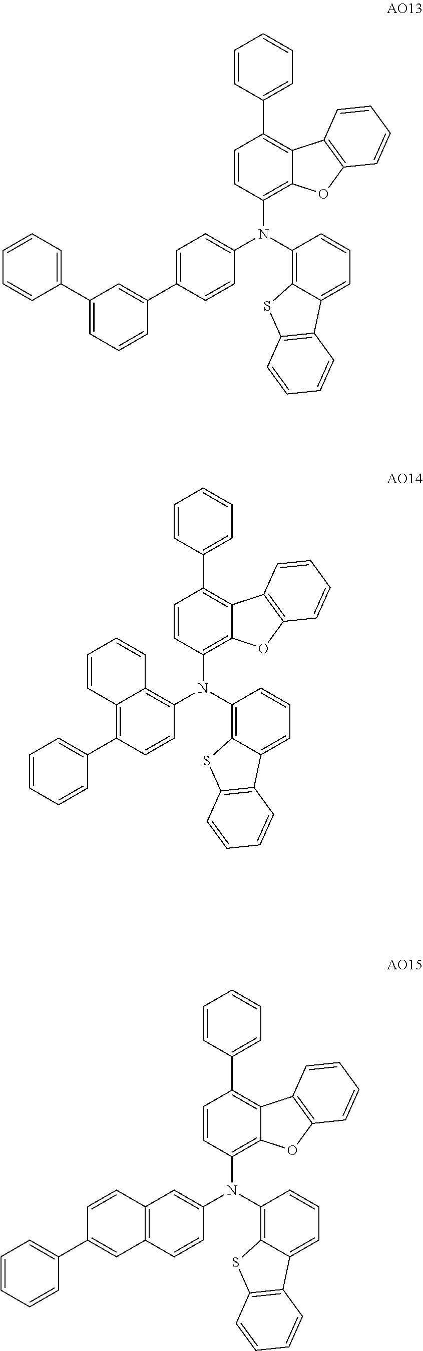 [SMILES: c1ccc(-c2cccc(-c3ccc(N(c4ccc(-c5ccccc5)c5c4oc4ccccc45)c4cccc5c4sc4ccccc45)cc3)c2)cc1 ; c1ccc(-c2ccc(N(c3ccc(-c4ccccc4)c4c3oc3ccccc34)c3cccc4c3sc3ccccc34)c3ccccc23)cc1 ; c1ccc(-c2ccc3cc(N(c4ccc(-c5ccccc5)c5c4oc4ccccc45)c4cccc5c4sc4ccccc45)ccc3c2)cc1] 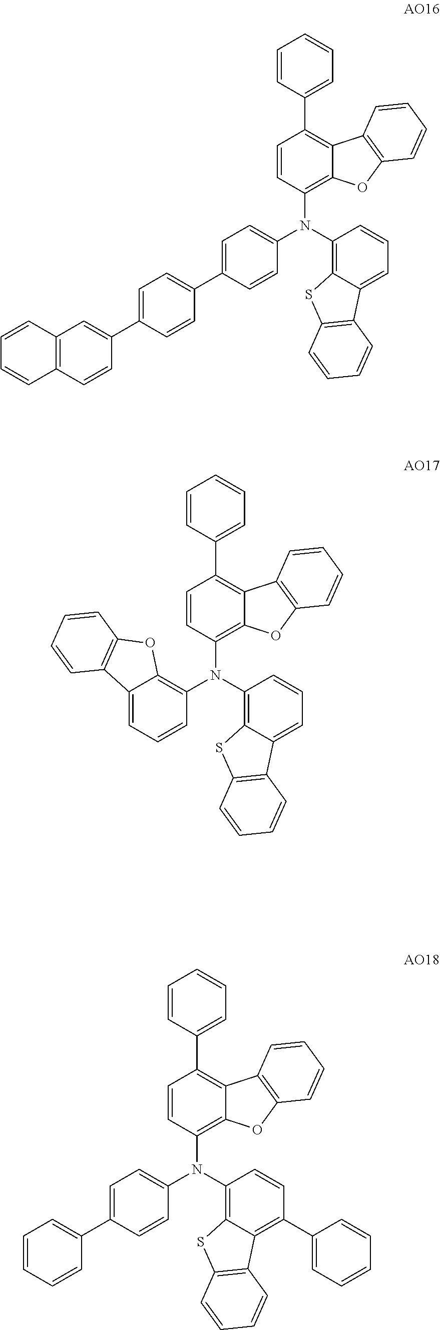 [SMILES: c1ccc(-c2ccc(N(c3ccc(-c4ccc(-c5ccc6ccccc6c5)cc4)cc3)c3cccc4c3sc3ccccc34)c3oc4ccccc4c23)cc1 ; c1ccc(-c2ccc(N(c3cccc4c3oc3ccccc34)c3cccc4c3sc3ccccc34)c3oc4ccccc4c23)cc1 ; c1ccc(-c2ccc(N(c3ccc(-c4ccccc4)c4c3oc3ccccc34)c3ccc(-c4ccccc4)c4c3sc3ccccc34)cc2)cc1] 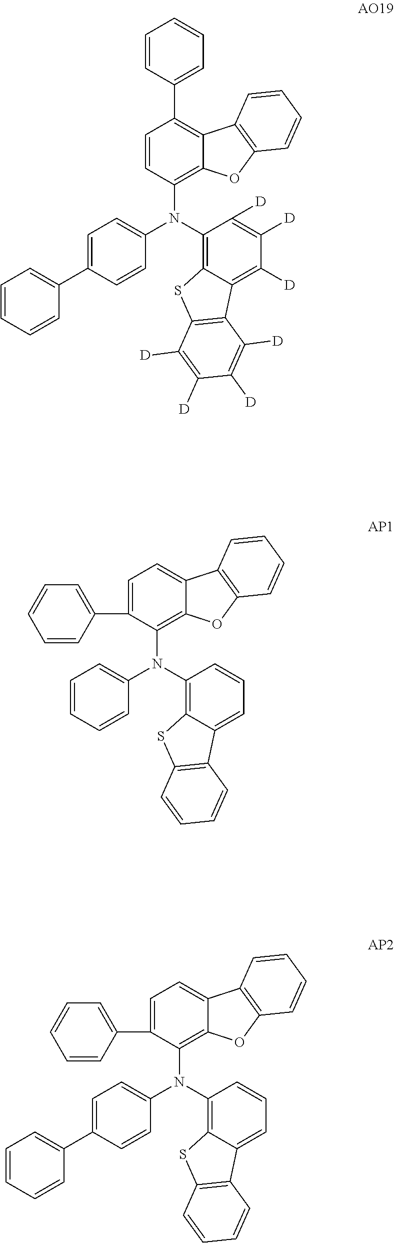 [SMILES: [2H]c1c([2H])c([2H])c2c(sc3c(N(c4ccc(-c5ccccc5)cc4)c4ccc(-c5ccccc5)c5c4oc4ccccc45)c([2H])c([2H])c([2H])c32)c1[2H] ; c1ccc(-c2ccc3c(oc4ccccc43)c2N(c2ccccc2)c2cccc3c2sc2ccccc23)cc1 ; c1ccc(-c2ccc(N(c3c(-c4ccccc4)ccc4c3oc3ccccc34)c3cccc4c3sc3ccccc34)cc2)cc1] 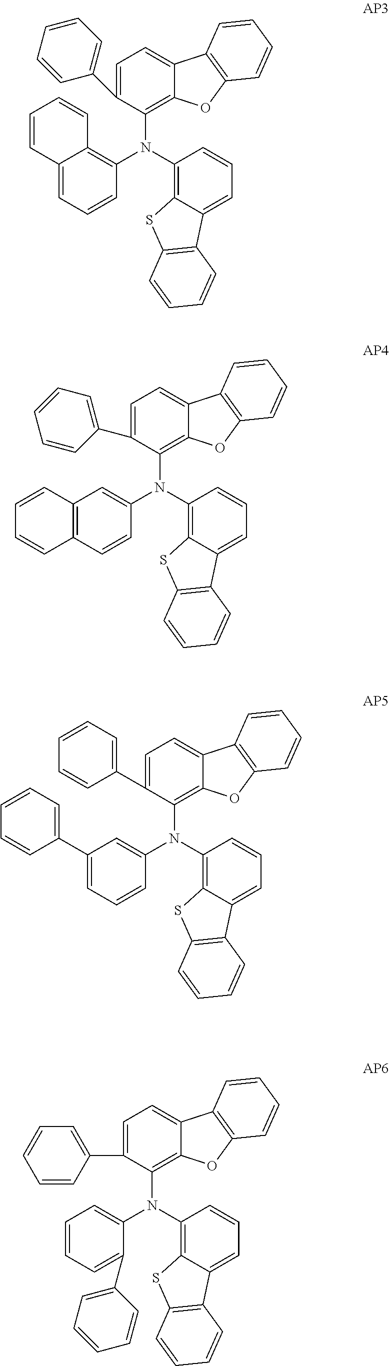 [SMILES: c1ccc(-c2ccc3c(oc4ccccc43)c2N(c2cccc3ccccc23)c2cccc3c2sc2ccccc23)cc1 ; c1ccc(-c2ccc3c(oc4ccccc43)c2N(c2ccc3ccccc3c2)c2cccc3c2sc2ccccc23)cc1 ; c1ccc(-c2cccc(N(c3c(-c4ccccc4)ccc4c3oc3ccccc34)c3cccc4c3sc3ccccc34)c2)cc1 ; c1ccc(-c2ccccc2N(c2c(-c3ccccc3)ccc3c2oc2ccccc23)c2cccc3c2sc2ccccc23)cc1] 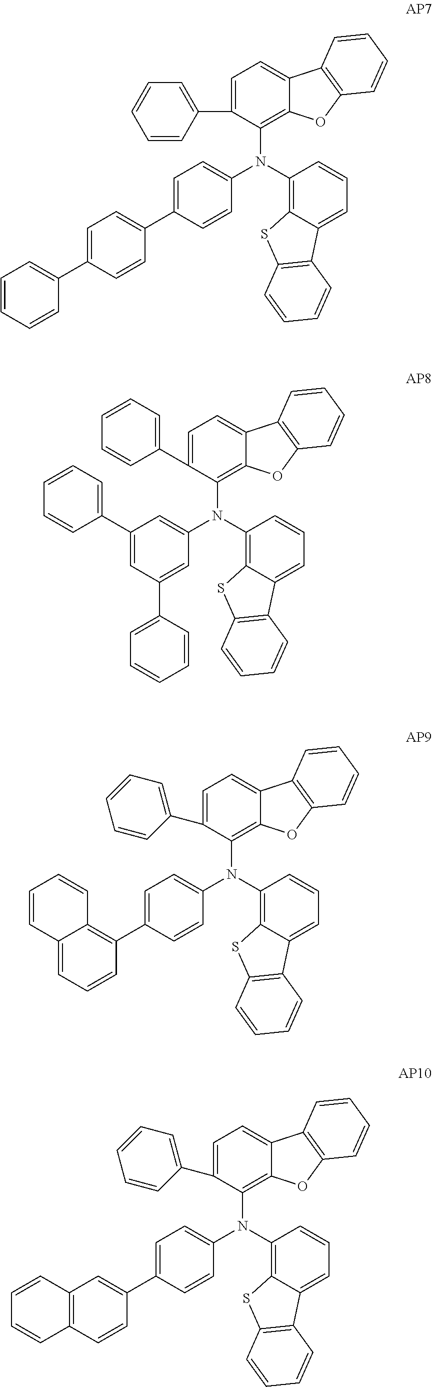 [SMILES: c1ccc(-c2ccc(-c3ccc(N(c4c(-c5ccccc5)ccc5c4oc4ccccc45)c4cccc5c4sc4ccccc45)cc3)cc2)cc1 ; c1ccc(-c2cc(-c3ccccc3)cc(N(c3c(-c4ccccc4)ccc4c3oc3ccccc34)c3cccc4c3sc3ccccc34)c2)cc1 ; c1ccc(-c2ccc3c(oc4ccccc43)c2N(c2ccc(-c3cccc4ccccc34)cc2)c2cccc3c2sc2ccccc23)cc1 ; c1ccc(-c2ccc3c(oc4ccccc43)c2N(c2ccc(-c3ccc4ccccc4c3)cc2)c2cccc3c2sc2ccccc23)cc1] 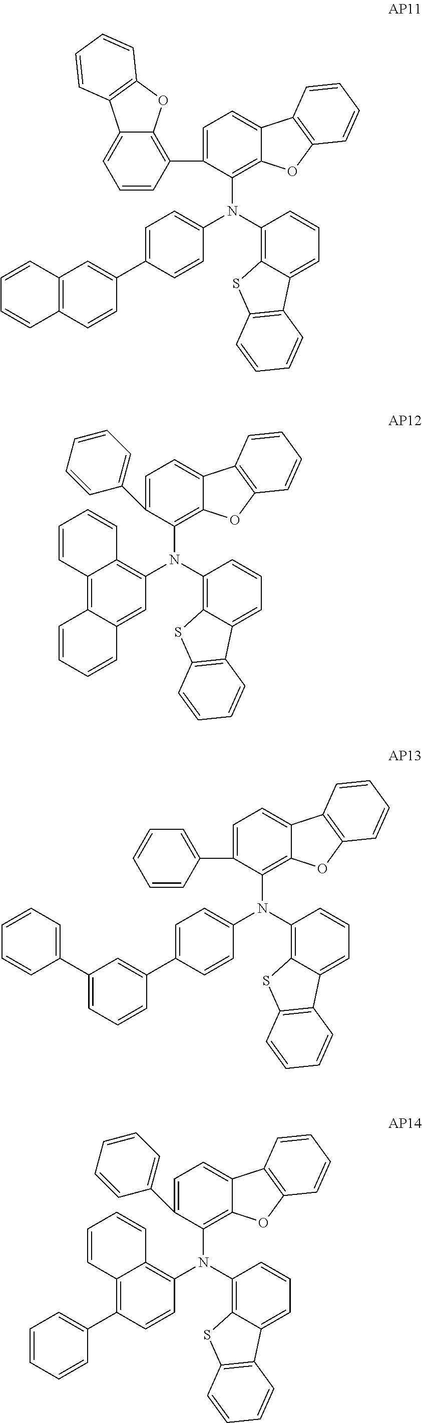 [SMILES: c1ccc2cc(-c3ccc(N(c4c(-c5cccc6c5oc5ccccc56)ccc5c4oc4ccccc45)c4cccc5c4sc4ccccc45)cc3)ccc2c1 ; c1ccc(-c2ccc3c(oc4ccccc43)c2N(c2cc3ccccc3c3ccccc23)c2cccc3c2sc2ccccc23)cc1 ; c1ccc(-c2cccc(-c3ccc(N(c4c(-c5ccccc5)ccc5c4oc4ccccc45)c4cccc5c4sc4ccccc45)cc3)c2)cc1 ; c1ccc(-c2ccc3c(oc4ccccc43)c2N(c2ccc(-c3ccccc3)c3ccccc23)c2cccc3c2sc2ccccc23)cc1] 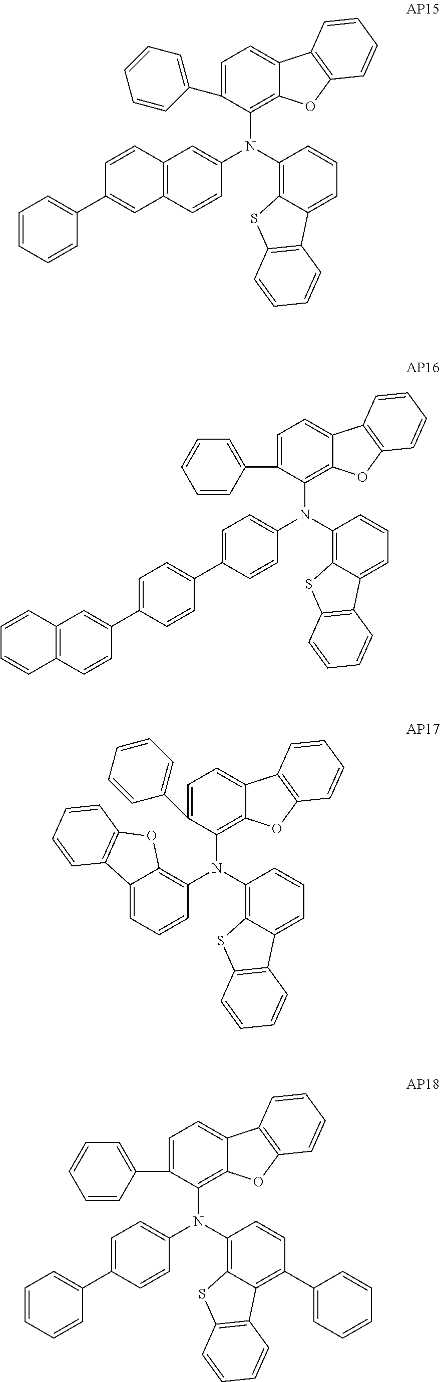 [SMILES: c1ccc(-c2ccc3cc(N(c4c(-c5ccccc5)ccc5c4oc4ccccc45)c4cccc5c4sc4ccccc45)ccc3c2)cc1 ; c1ccc(-c2ccc3c(oc4ccccc43)c2N(c2ccc(-c3ccc(-c4ccc5ccccc5c4)cc3)cc2)c2cccc3c2sc2ccccc23)cc1 ; c1ccc(-c2ccc3c(oc4ccccc43)c2N(c2cccc3c2oc2ccccc23)c2cccc3c2sc2ccccc23)cc1 ; c1ccc(-c2ccc(N(c3c(-c4ccccc4)ccc4c3oc3ccccc34)c3ccc(-c4ccccc4)c4c3sc3ccccc34)cc2)cc1] 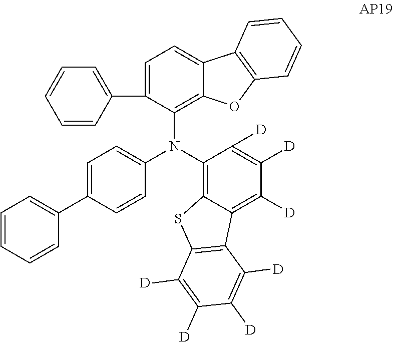 [SMILES: [2H]c1c([2H])c([2H])c2c(sc3c(N(c4ccc(-c5ccccc5)cc4)c4c(-c5ccccc5)ccc5c4oc4ccccc45)c([2H])c([2H])c([2H])c32)c1[2H]] 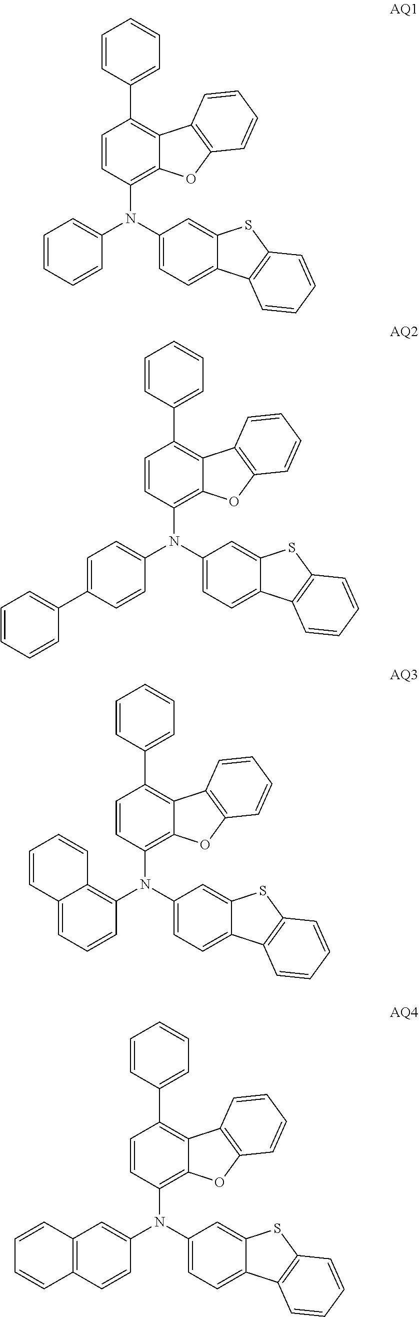 [SMILES: c1ccc(-c2ccc(N(c3ccccc3)c3ccc4c(c3)sc3ccccc34)c3oc4ccccc4c23)cc1 ; c1ccc(-c2ccc(N(c3ccc4c(c3)sc3ccccc34)c3ccc(-c4ccccc4)c4c3oc3ccccc34)cc2)cc1 ; c1ccc(-c2ccc(N(c3ccc4c(c3)sc3ccccc34)c3cccc4ccccc34)c3oc4ccccc4c23)cc1 ; c1ccc(-c2ccc(N(c3ccc4ccccc4c3)c3ccc4c(c3)sc3ccccc34)c3oc4ccccc4c23)cc1] 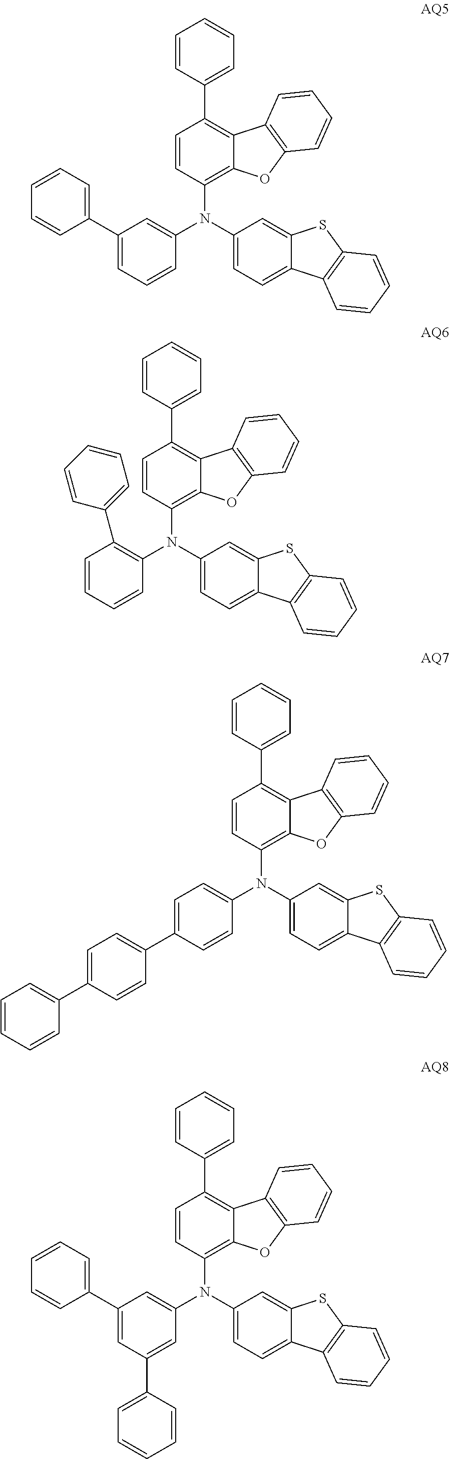 [SMILES: c1ccc(-c2cccc(N(c3ccc4c(c3)sc3ccccc34)c3ccc(-c4ccccc4)c4c3oc3ccccc34)c2)cc1 ; c1ccc(-c2ccccc2N(c2ccc3c(c2)sc2ccccc23)c2ccc(-c3ccccc3)c3c2oc2ccccc23)cc1 ; c1ccc(-c2ccc(-c3ccc(N(c4ccc5c(c4)sc4ccccc45)c4ccc(-c5ccccc5)c5c4oc4ccccc45)cc3)cc2)cc1 ; c1ccc(-c2cc(-c3ccccc3)cc(N(c3ccc4c(c3)sc3ccccc34)c3ccc(-c4ccccc4)c4c3oc3ccccc34)c2)cc1] 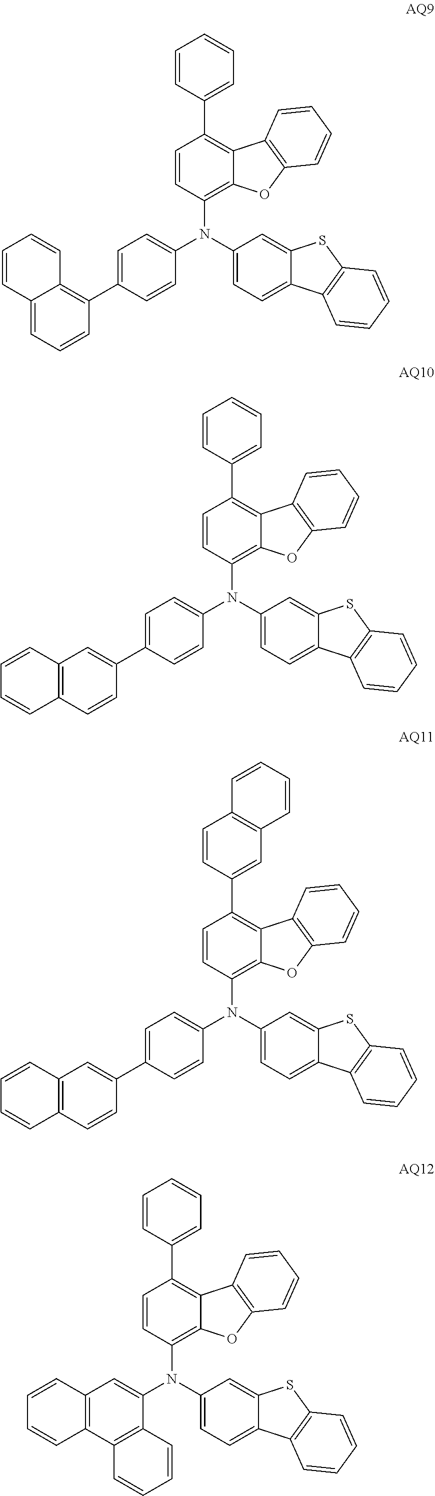 [SMILES: c1ccc(-c2ccc(N(c3ccc(-c4cccc5ccccc45)cc3)c3ccc4c(c3)sc3ccccc34)c3oc4ccccc4c23)cc1 ; c1ccc(-c2ccc(N(c3ccc(-c4ccc5ccccc5c4)cc3)c3ccc4c(c3)sc3ccccc34)c3oc4ccccc4c23)cc1 ; c1ccc2cc(-c3ccc(N(c4ccc5c(c4)sc4ccccc45)c4ccc(-c5ccc6ccccc6c5)c5c4oc4ccccc45)cc3)ccc2c1 ; c1ccc(-c2ccc(N(c3ccc4c(c3)sc3ccccc34)c3cc4ccccc4c4ccccc34)c3oc4ccccc4c23)cc1] 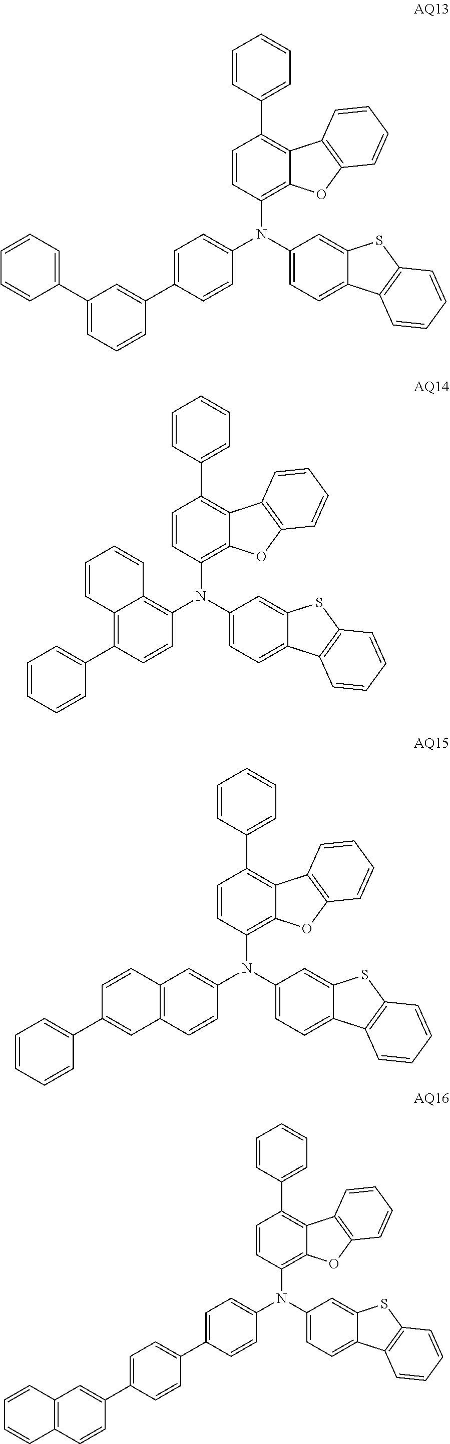 [SMILES: c1ccc(-c2cccc(-c3ccc(N(c4ccc5c(c4)sc4ccccc45)c4ccc(-c5ccccc5)c5c4oc4ccccc45)cc3)c2)cc1 ; c1ccc(-c2ccc(N(c3ccc4c(c3)sc3ccccc34)c3ccc(-c4ccccc4)c4c3oc3ccccc34)c3ccccc23)cc1 ; c1ccc(-c2ccc3cc(N(c4ccc5c(c4)sc4ccccc45)c4ccc(-c5ccccc5)c5c4oc4ccccc45)ccc3c2)cc1 ; c1ccc(-c2ccc(N(c3ccc(-c4ccc(-c5ccc6ccccc6c5)cc4)cc3)c3ccc4c(c3)sc3ccccc34)c3oc4ccccc4c23)cc1] 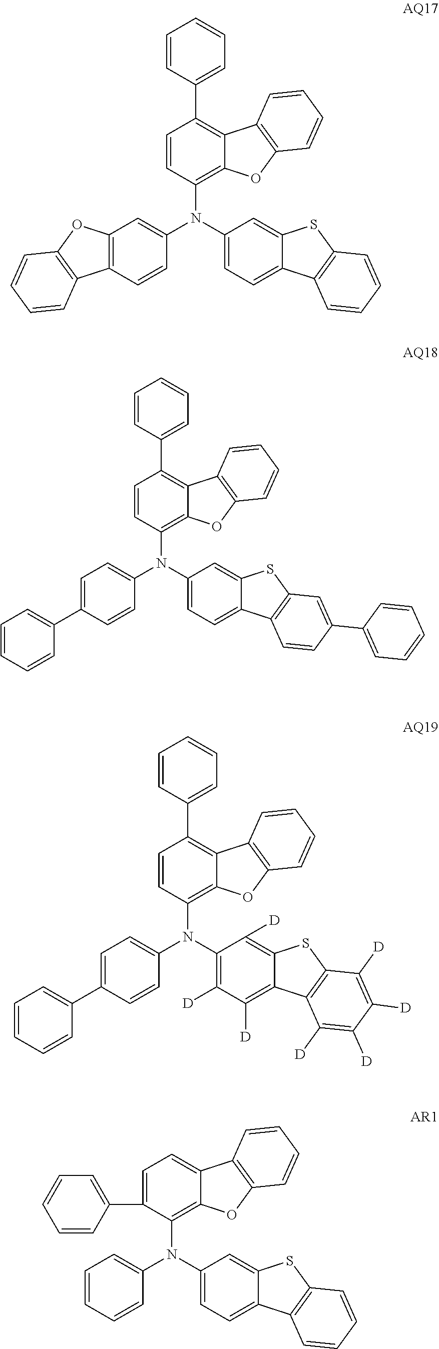 [SMILES: c1ccc(-c2ccc(N(c3ccc4c(c3)oc3ccccc34)c3ccc4c(c3)sc3ccccc34)c3oc4ccccc4c23)cc1 ; c1ccc(-c2ccc(N(c3ccc4c(c3)sc3cc(-c5ccccc5)ccc34)c3ccc(-c4ccccc4)c4c3oc3ccccc34)cc2)cc1 ; [2H]c1c([2H])c([2H])c2c(sc3c([2H])c(N(c4ccc(-c5ccccc5)cc4)c4ccc(-c5ccccc5)c5c4oc4ccccc45)c([2H])c([2H])c32)c1[2H] ; c1ccc(-c2ccc3c(oc4ccccc43)c2N(c2ccccc2)c2ccc3c(c2)sc2ccccc23)cc1] 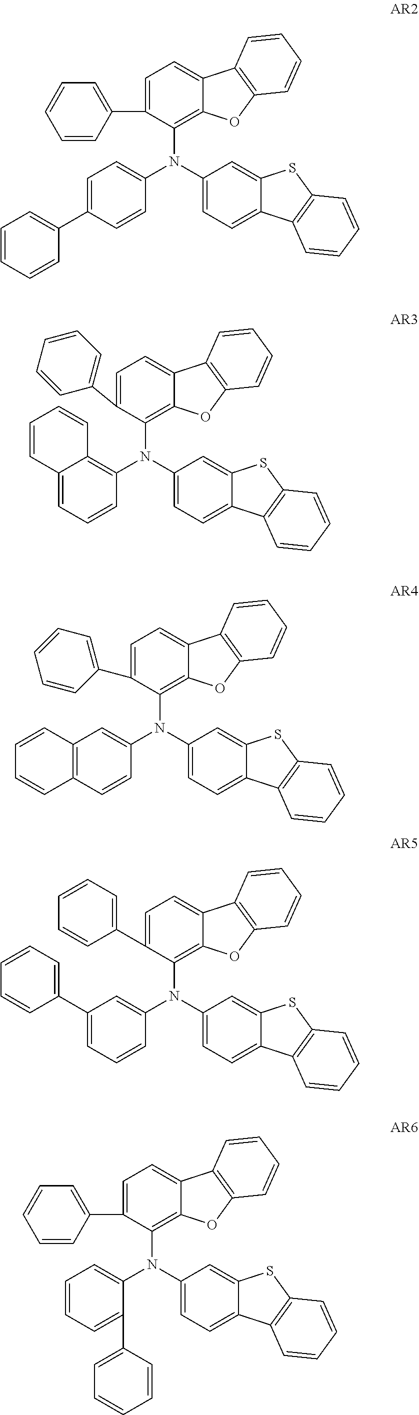 [SMILES: c1ccc(-c2ccc(N(c3ccc4c(c3)sc3ccccc34)c3c(-c4ccccc4)ccc4c3oc3ccccc34)cc2)cc1 ; c1ccc(-c2ccc3c(oc4ccccc43)c2N(c2ccc3c(c2)sc2ccccc23)c2cccc3ccccc23)cc1 ; c1ccc(-c2ccc3c(oc4ccccc43)c2N(c2ccc3ccccc3c2)c2ccc3c(c2)sc2ccccc23)cc1 ; c1ccc(-c2cccc(N(c3ccc4c(c3)sc3ccccc34)c3c(-c4ccccc4)ccc4c3oc3ccccc34)c2)cc1 ; c1ccc(-c2ccccc2N(c2ccc3c(c2)sc2ccccc23)c2c(-c3ccccc3)ccc3c2oc2ccccc23)cc1] 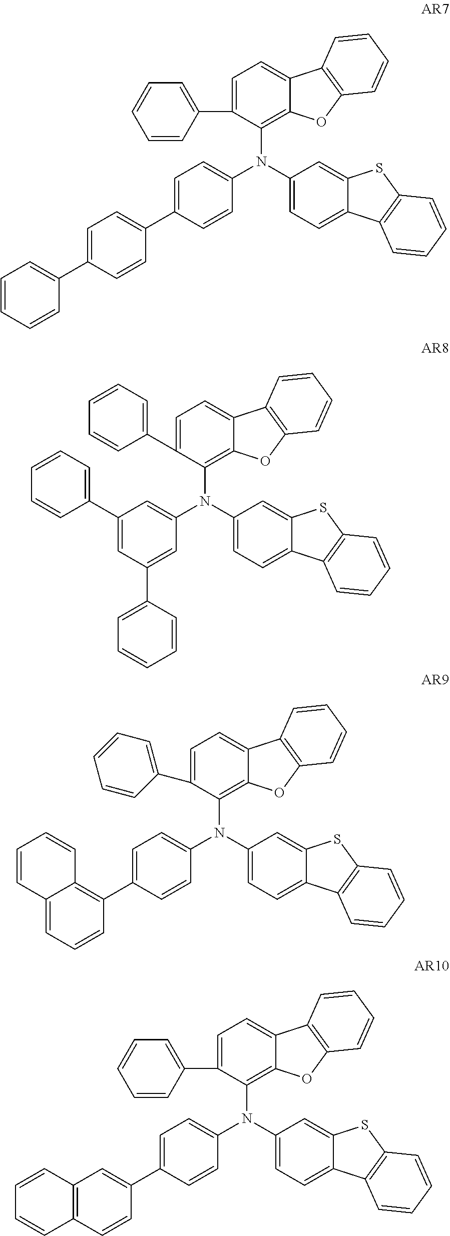 [SMILES: c1ccc(-c2ccc(-c3ccc(N(c4ccc5c(c4)sc4ccccc45)c4c(-c5ccccc5)ccc5c4oc4ccccc45)cc3)cc2)cc1 ; c1ccc(-c2cc(-c3ccccc3)cc(N(c3ccc4c(c3)sc3ccccc34)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc3c(c2)sc2ccccc23)cc1 ; c1ccc(-c2ccc3c(oc4ccccc43)c2N(c2ccc(-c3ccc4ccccc4c3)cc2)c2ccc3c(c2)sc2ccccc23)cc1] 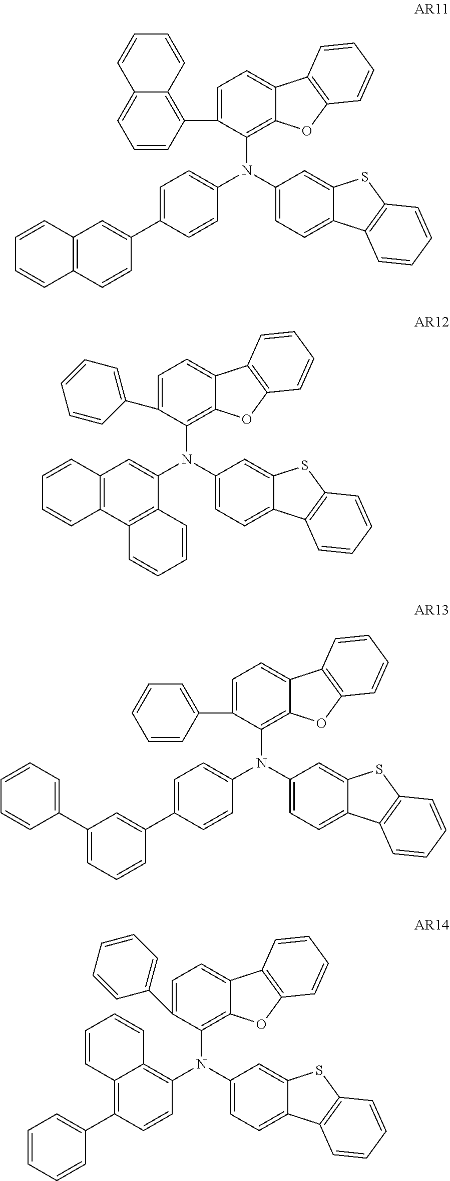 [SMILES: c1ccc2cc(-c3ccc(N(c4ccc5c(c4)sc4ccccc45)c4c(-c5cccc6ccccc56)ccc5c4oc4ccccc45)cc3)ccc2c1 ; c1ccc(-c2ccc3c(oc4ccccc43)c2N(c2ccc3c(c2)sc2ccccc23)c2cc3ccccc3c3ccccc23)cc1 ; c1ccc(-c2cccc(-c3ccc(N(c4ccc5c(c4)sc4ccccc45)c4c(-c5ccccc5)ccc5c4oc4ccccc45)cc3)c2)cc1 ; c1ccc(-c2ccc3c(oc4ccccc43)c2N(c2ccc3c(c2)sc2ccccc23)c2ccc(-c3ccccc3)c3ccccc23)cc1] 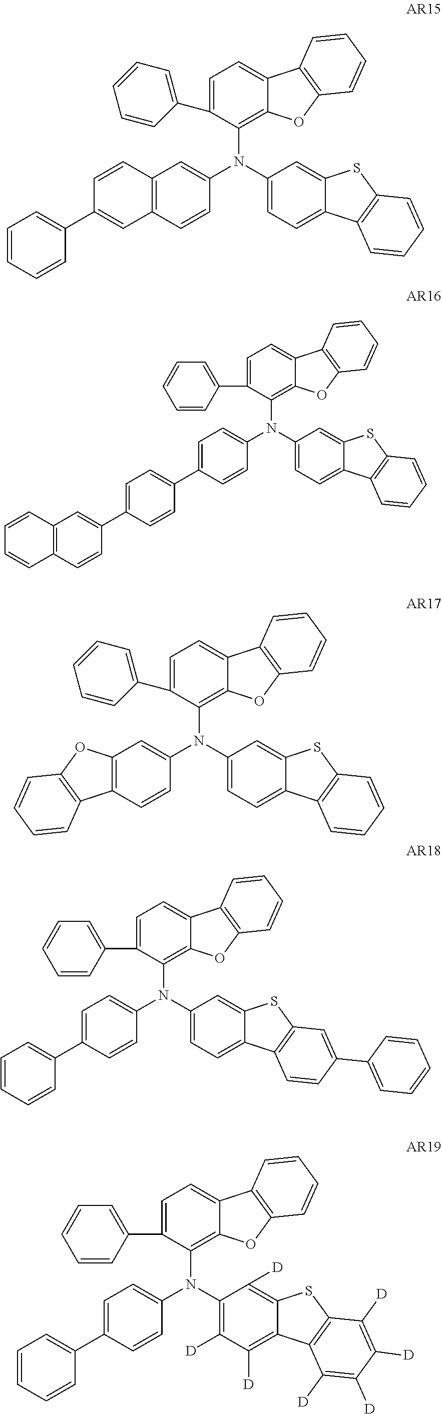 [SMILES: c1ccc(-c2ccc3cc(N(c4ccc5c(c4)sc4ccccc45)c4c(-c5ccccc5)ccc5c4oc4ccccc45)ccc3c2)cc1 ; c1ccc(-c2ccc3c(oc4ccccc43)c2N(c2ccc(-c3ccc(-c4ccc5ccccc5c4)cc3)cc2)c2ccc3c(c2)sc2ccccc23)cc1 ; c1ccc(-c2ccc3c(oc4ccccc43)c2N(c2ccc3c(c2)oc2ccccc23)c2ccc3c(c2)sc2ccccc23)cc1 ; c1ccc(-c2ccc(N(c3ccc4c(c3)sc3cc(-c5ccccc5)ccc34)c3c(-c4ccccc4)ccc4c3oc3ccccc34)cc2)cc1 ; [2H]c1c([2H])c([2H])c2c(sc3c([2H])c(N(c4ccc(-c5ccccc5)cc4)c4c(-c5ccccc5)ccc5c4oc4ccccc45)c([2H])c([2H])c32)c1[2H]] 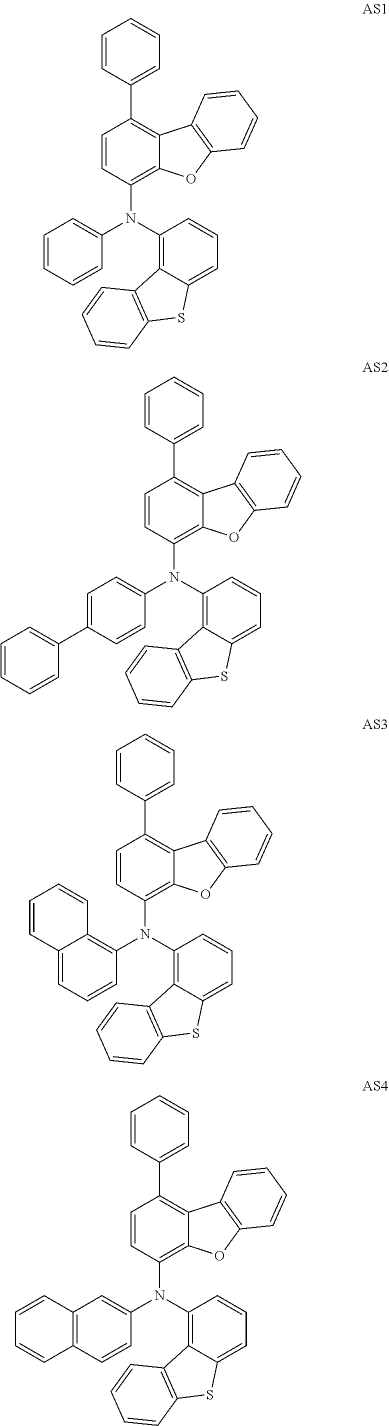 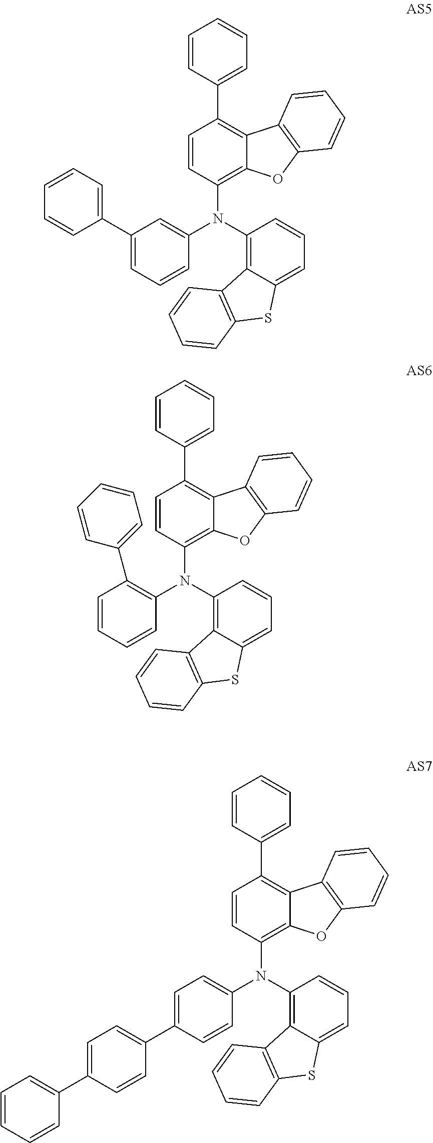 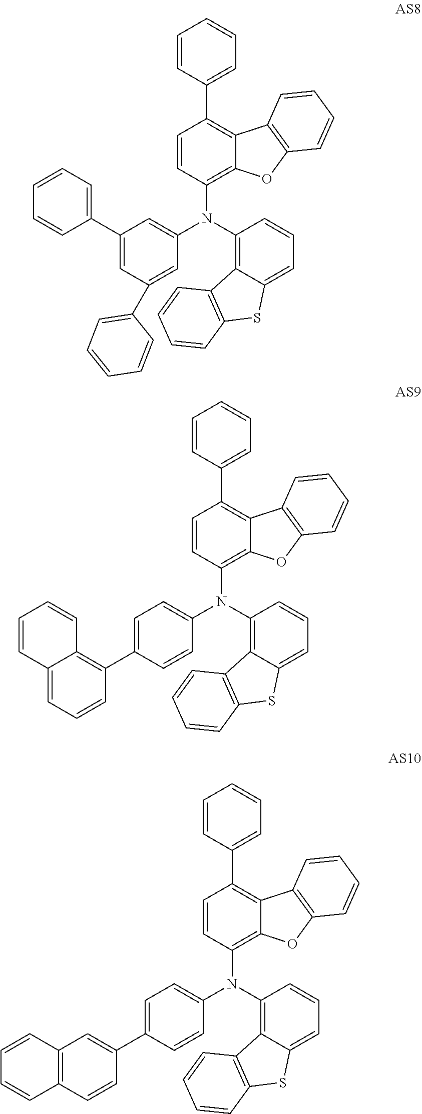 [SMILES: c1ccc(-c2cc(-c3ccccc3)cc(N(c3ccc(-c4ccccc4)c4c3oc3ccccc34)c3cccc4sc5ccccc5c34)c2)cc1 ; c1ccc(-c2ccc(N(c3ccc(-c4cccc5ccccc45)cc3)c3cccc4sc5ccccc5c34)c3oc4ccccc4c23)cc1 ; c1ccc(-c2ccc(N(c3ccc(-c4ccc5ccccc5c4)cc3)c3cccc4sc5ccccc5c34)c3oc4ccccc4c23)cc1] 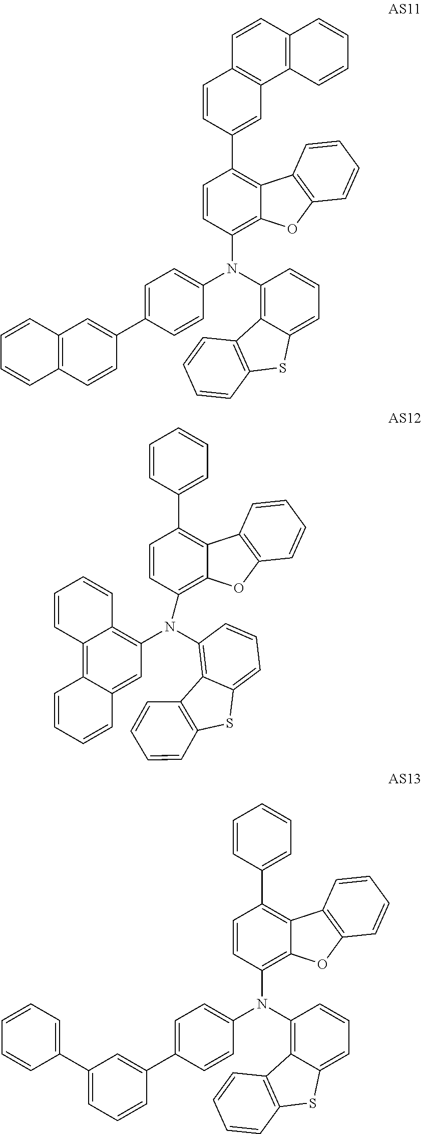 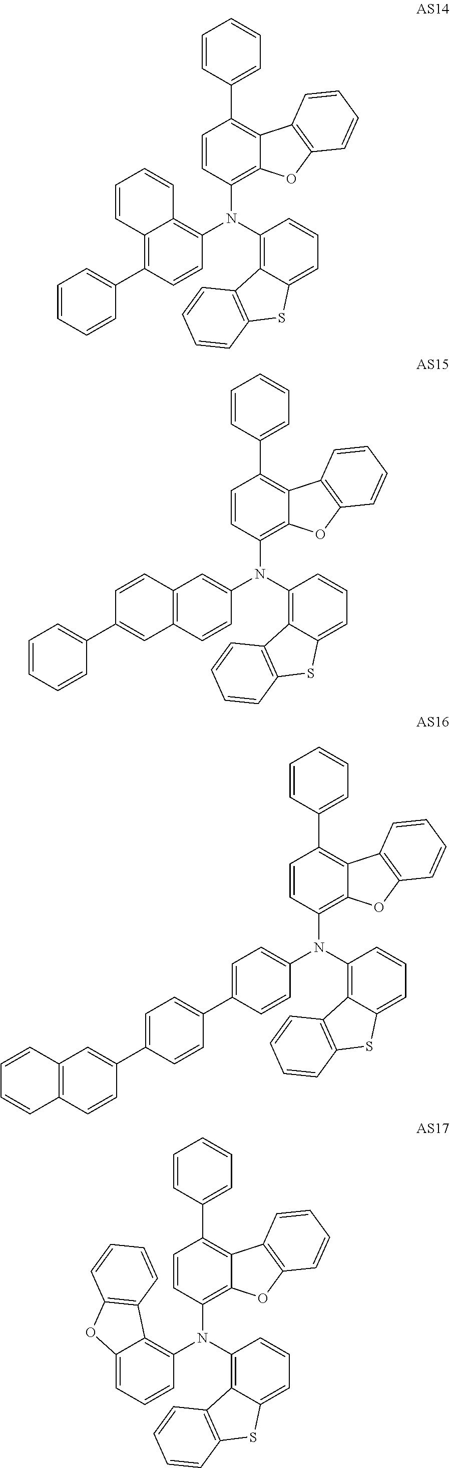 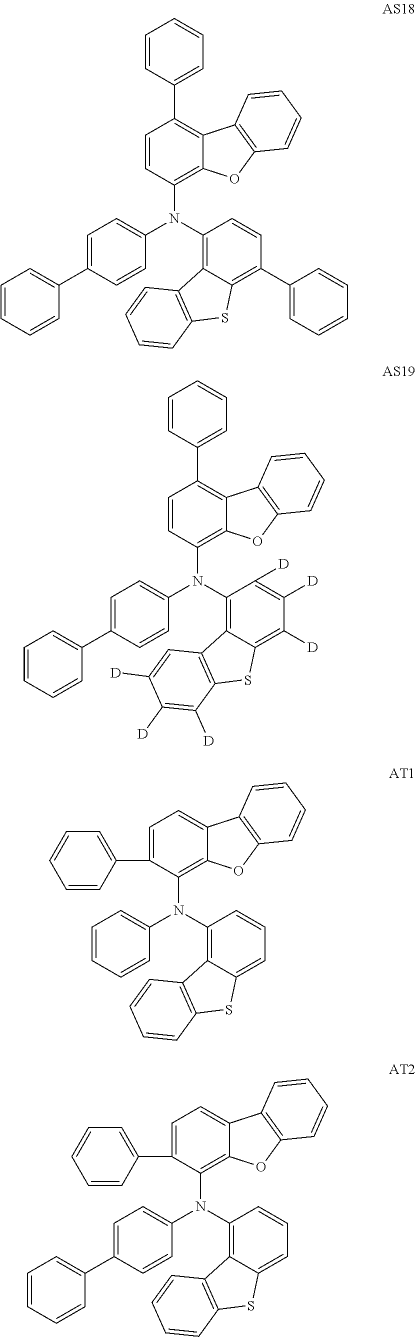 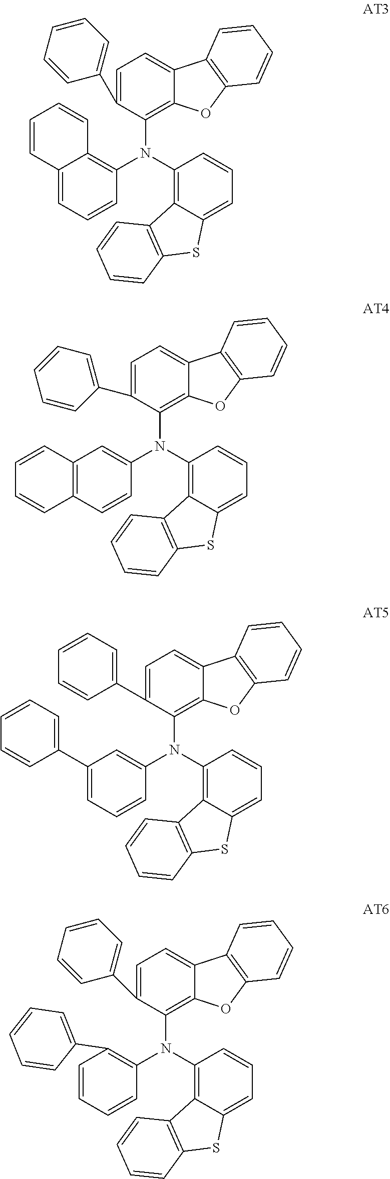 [SMILES: c1ccc(-c2ccc3c(oc4ccccc43)c2N(c2cccc3ccccc23)c2cccc3sc4ccccc4c23)cc1 ; c1ccc(-c2ccc3c(oc4ccccc43)c2N(c2ccc3ccccc3c2)c2cccc3sc4ccccc4c23)cc1 ; c1ccc(-c2cccc(N(c3c(-c4ccccc4)ccc4c3oc3ccccc34)c3cccc4sc5ccccc5c34)c2)cc1 ; c1ccc(-c2ccccc2N(c2c(-c3ccccc3)ccc3c2oc2ccccc23)c2cccc3sc4ccccc4c23)cc1] 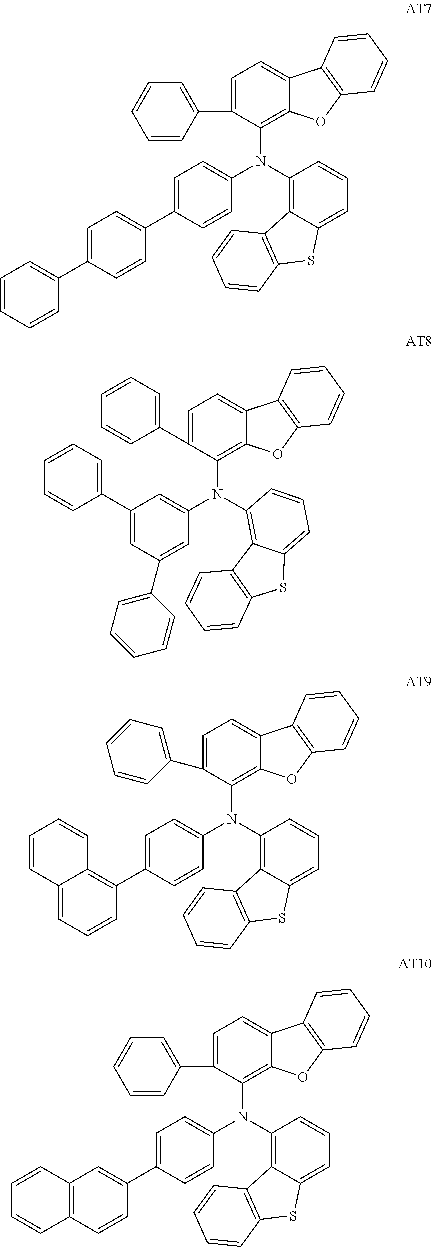 [SMILES: c1ccc(-c2ccc(-c3ccc(N(c4c(-c5ccccc5)ccc5c4oc4ccccc45)c4cccc5sc6ccccc6c45)cc3)cc2)cc1 ; c1ccc(-c2cc(-c3ccccc3)cc(N(c3c(-c4ccccc4)ccc4c3oc3ccccc34)c3cccc4sc5ccccc5c34)c2)cc1 ; c1ccc(-c2ccc3c(oc4ccccc43)c2N(c2ccc(-c3cccc4ccccc34)cc2)c2cccc3sc4ccccc4c23)cc1 ; c1ccc(-c2ccc3c(oc4ccccc43)c2N(c2ccc(-c3ccc4ccccc4c3)cc2)c2cccc3sc4ccccc4c23)cc1] 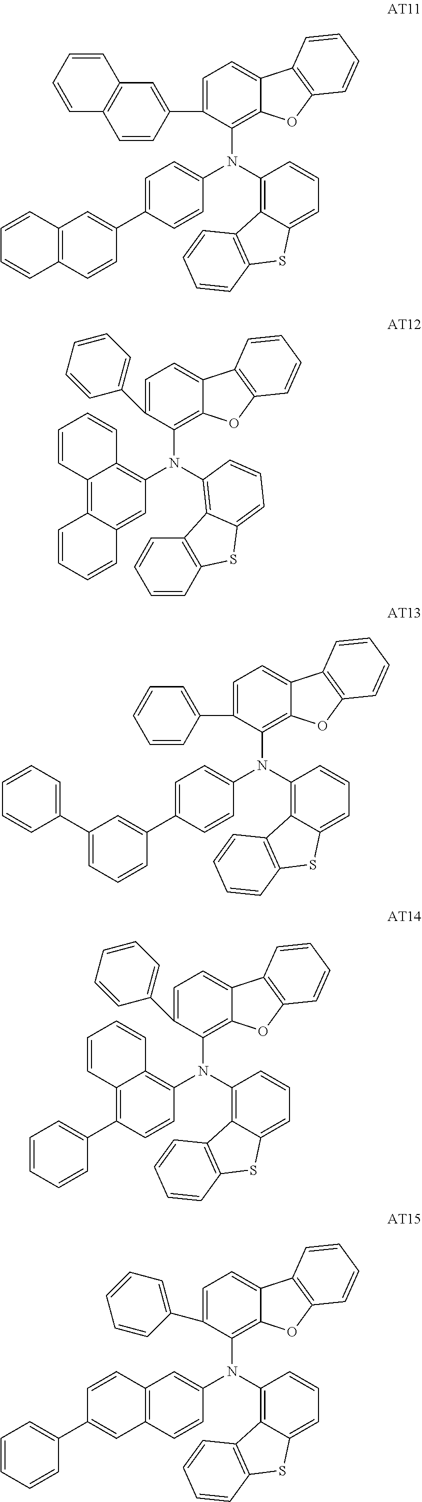 [SMILES: c1ccc2cc(-c3ccc(N(c4c(-c5ccc6ccccc6c5)ccc5c4oc4ccccc45)c4cccc5sc6ccccc6c45)cc3)ccc2c1 ; c1ccc(-c2ccc3c(oc4ccccc43)c2N(c2cc3ccccc3c3ccccc23)c2cccc3sc4ccccc4c23)cc1 ; c1ccc(-c2cccc(-c3ccc(N(c4c(-c5ccccc5)ccc5c4oc4ccccc45)c4cccc5sc6ccccc6c45)cc3)c2)cc1 ; c1ccc(-c2ccc3c(oc4ccccc43)c2N(c2ccc(-c3ccccc3)c3ccccc23)c2cccc3sc4ccccc4c23)cc1 ; c1ccc(-c2ccc3cc(N(c4c(-c5ccccc5)ccc5c4oc4ccccc45)c4cccc5sc6ccccc6c45)ccc3c2)cc1] 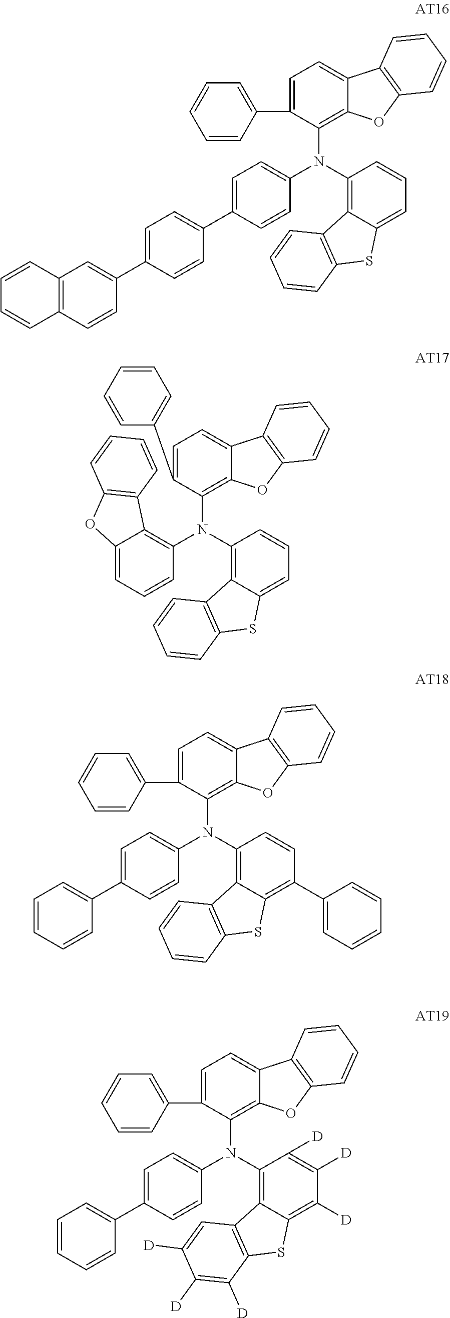 [SMILES: c1ccc(-c2ccc3c(oc4ccccc43)c2N(c2ccc(-c3ccc(-c4ccc5ccccc5c4)cc3)cc2)c2cccc3sc4ccccc4c23)cc1 ; c1ccc(-c2ccc3c(oc4ccccc43)c2N(c2cccc3oc4ccccc4c23)c2cccc3sc4ccccc4c23)cc1 ; c1ccc(-c2ccc(N(c3c(-c4ccccc4)ccc4c3oc3ccccc34)c3ccc(-c4ccccc4)c4sc5ccccc5c34)cc2)cc1 ; [2H]c1cc2c(sc3c([2H])c([2H])c([2H])c(N(c4ccc(-c5ccccc5)cc4)c4c(-c5ccccc5)ccc5c4oc4ccccc45)c32)c([2H])c1[2H]] 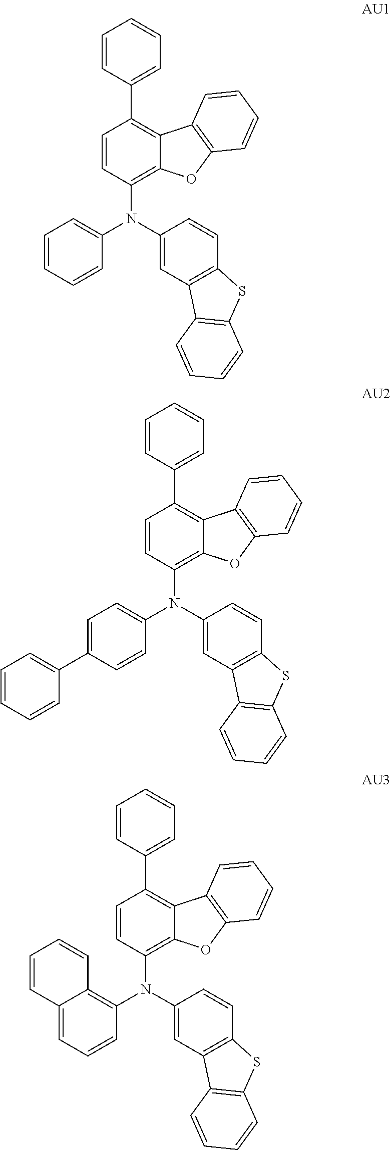 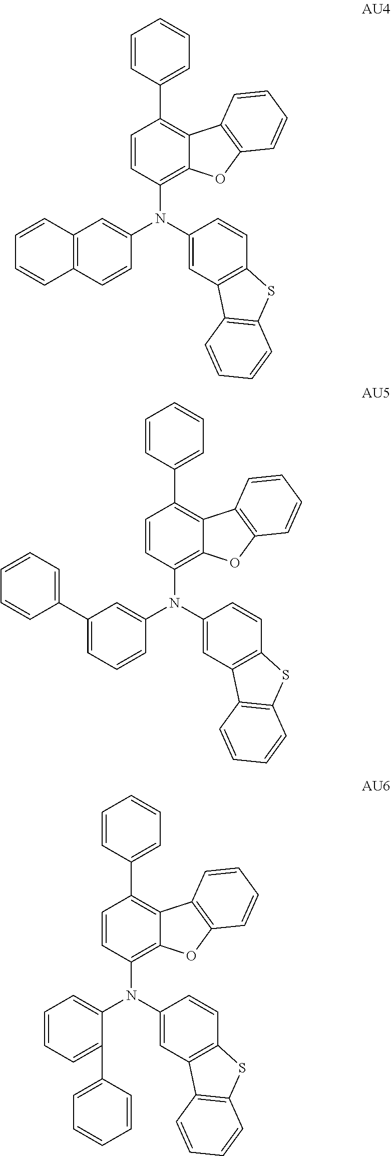 [SMILES: c1ccc(-c2ccc(N(c3ccc4ccccc4c3)c3ccc4sc5ccccc5c4c3)c3oc4ccccc4c23)cc1 ; c1ccc(-c2cccc(N(c3ccc4sc5ccccc5c4c3)c3ccc(-c4ccccc4)c4c3oc3ccccc34)c2)cc1 ; c1ccc(-c2ccccc2N(c2ccc3sc4ccccc4c3c2)c2ccc(-c3ccccc3)c3c2oc2ccccc23)cc1] 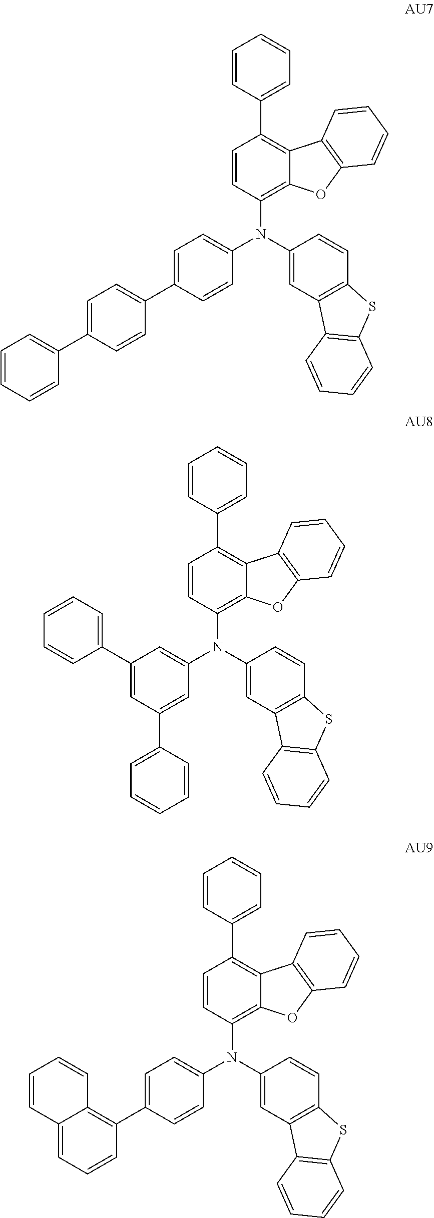 [SMILES: c1ccc(-c2ccc(-c3ccc(N(c4ccc5sc6ccccc6c5c4)c4ccc(-c5ccccc5)c5c4oc4ccccc45)cc3)cc2)cc1 ; c1ccc(-c2cc(-c3ccccc3)cc(N(c3ccc4sc5ccccc5c4c3)c3ccc(-c4ccccc4)c4c3oc3ccccc34)c2)cc1 ; c1ccc(-c2ccc(N(c3ccc(-c4cccc5ccccc45)cc3)c3ccc4sc5ccccc5c4c3)c3oc4ccccc4c23)cc1] 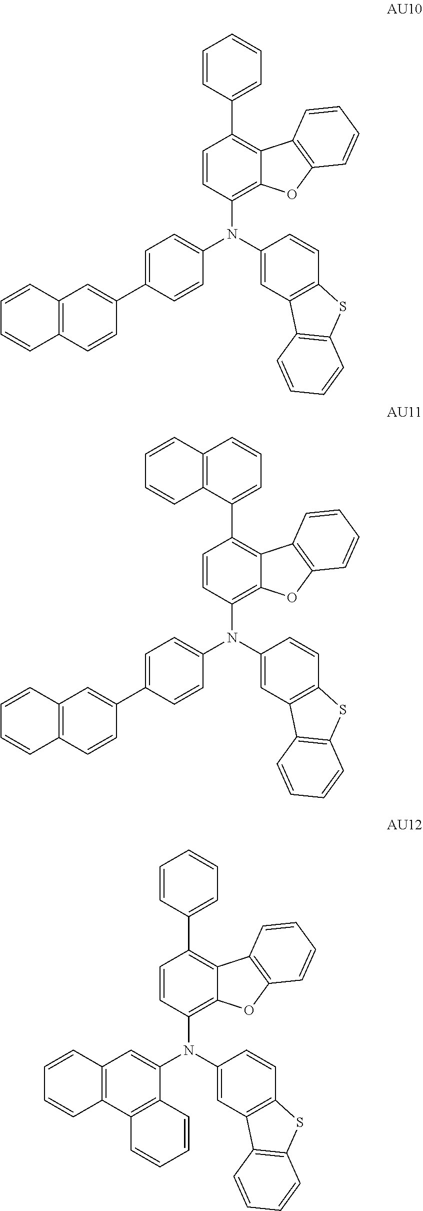 [SMILES: c1ccc(-c2ccc(N(c3ccc(-c4ccc5ccccc5c4)cc3)c3ccc4sc5ccccc5c4c3)c3oc4ccccc4c23)cc1 ; c1ccc2cc(-c3ccc(N(c4ccc5sc6ccccc6c5c4)c4ccc(-c5cccc6ccccc56)c5c4oc4ccccc45)cc3)ccc2c1 ; c1ccc(-c2ccc(N(c3ccc4sc5ccccc5c4c3)c3cc4ccccc4c4ccccc34)c3oc4ccccc4c23)cc1] 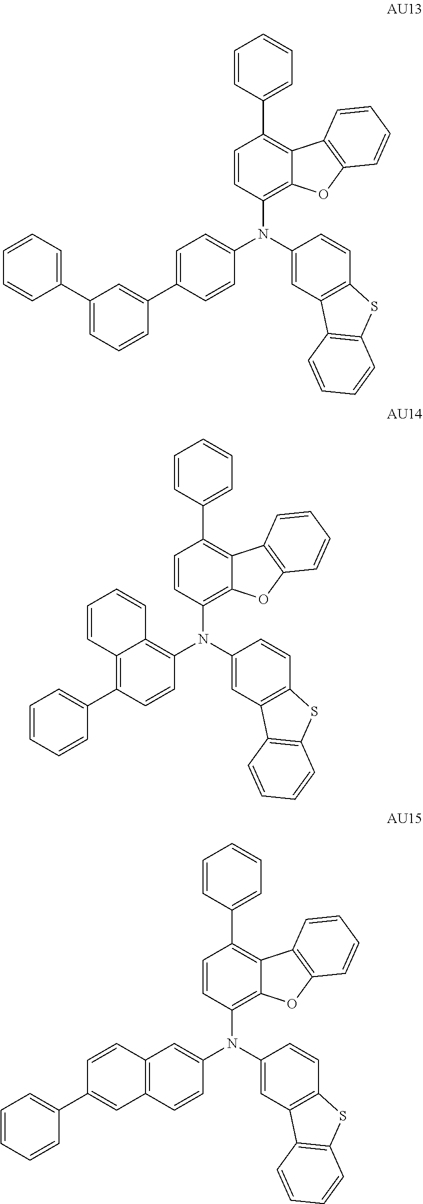 [SMILES: c1ccc(-c2cccc(-c3ccc(N(c4ccc5sc6ccccc6c5c4)c4ccc(-c5ccccc5)c5c4oc4ccccc45)cc3)c2)cc1 ; c1ccc(-c2ccc(N(c3ccc4sc5ccccc5c4c3)c3ccc(-c4ccccc4)c4c3oc3ccccc34)c3ccccc23)cc1 ; c1ccc(-c2ccc3cc(N(c4ccc5sc6ccccc6c5c4)c4ccc(-c5ccccc5)c5c4oc4ccccc45)ccc3c2)cc1] 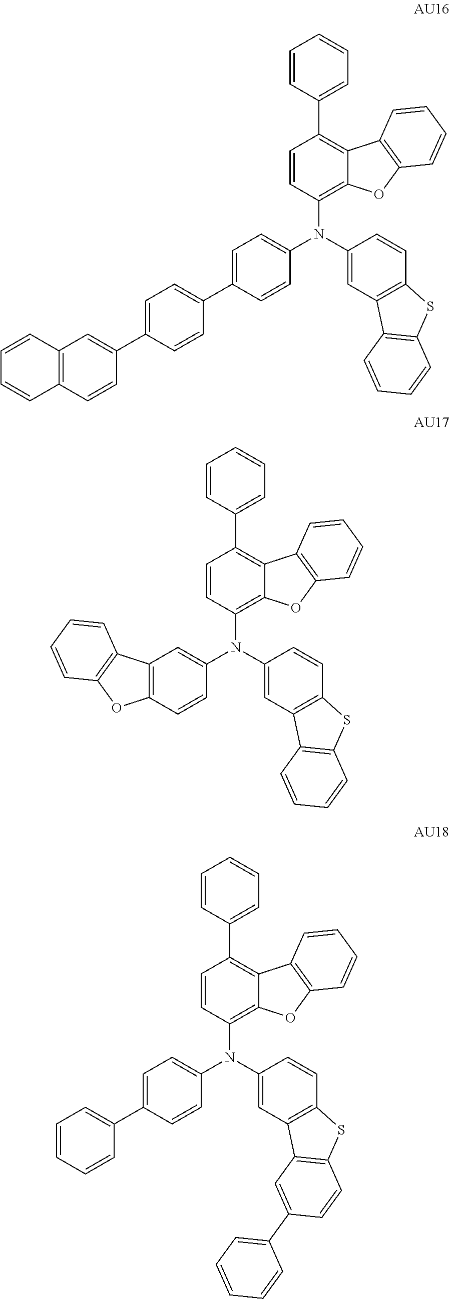 [SMILES: c1ccc(-c2ccc(N(c3ccc(-c4ccc(-c5ccc6ccccc6c5)cc4)cc3)c3ccc4sc5ccccc5c4c3)c3oc4ccccc4c23)cc1 ; c1ccc(-c2ccc(N(c3ccc4oc5ccccc5c4c3)c3ccc4sc5ccccc5c4c3)c3oc4ccccc4c23)cc1 ; c1ccc(-c2ccc(N(c3ccc4sc5ccc(-c6ccccc6)cc5c4c3)c3ccc(-c4ccccc4)c4c3oc3ccccc34)cc2)cc1] 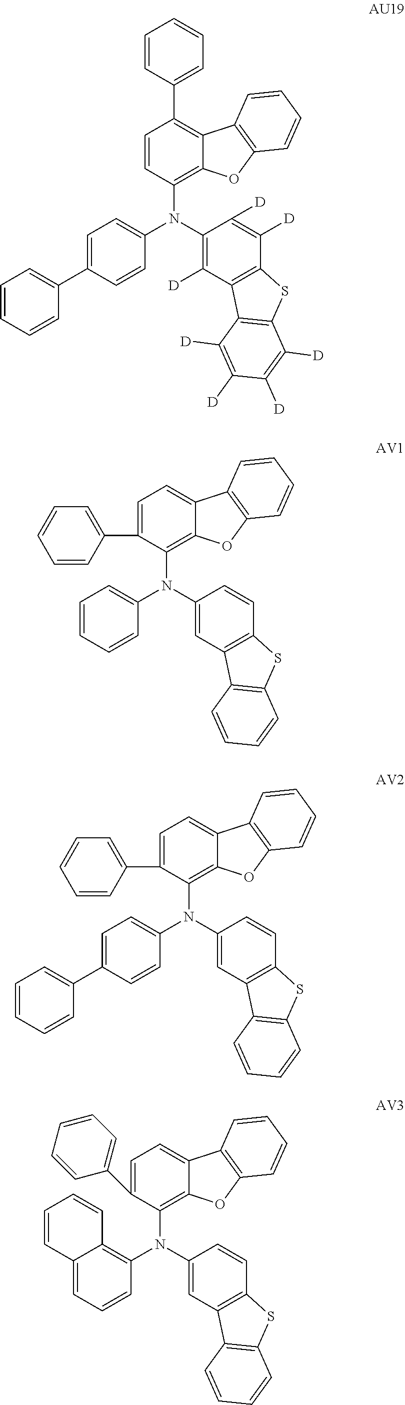 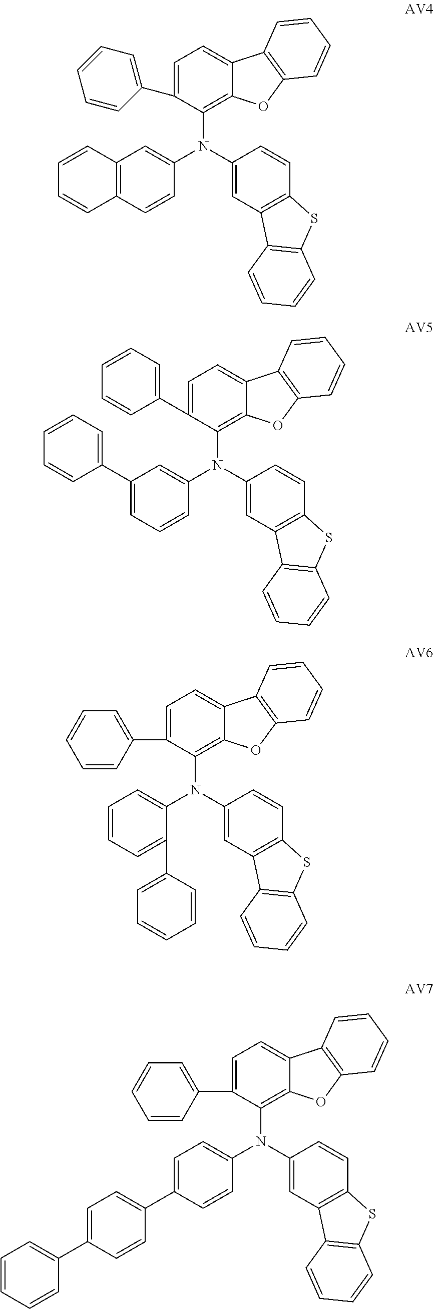 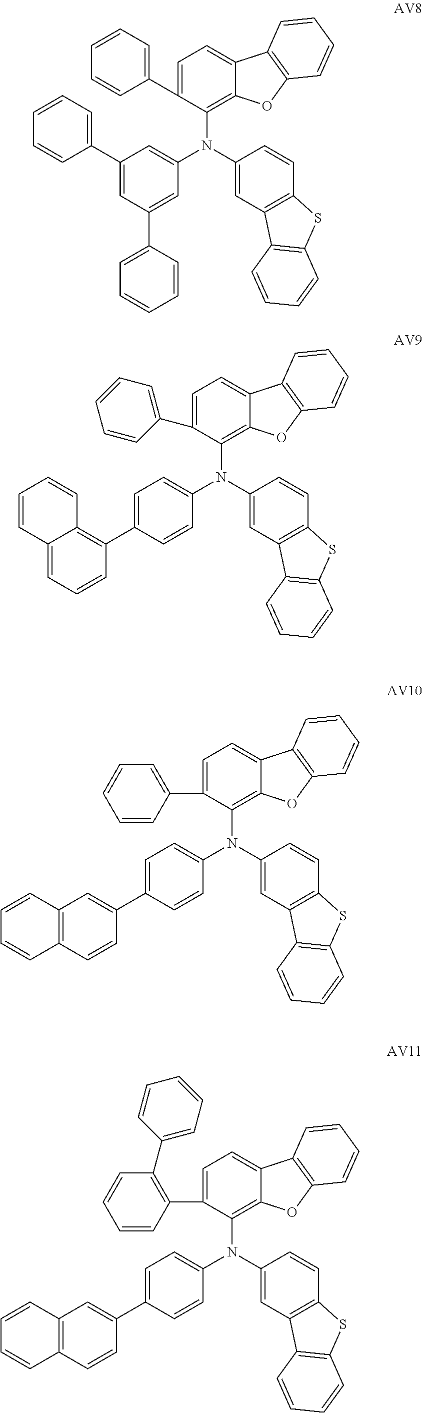 [SMILES: c1ccc(-c2cc(-c3ccccc3)cc(N(c3ccc4sc5ccccc5c4c3)c3c(-c4ccccc4)ccc4c3oc3ccccc34)c2)cc1 ; c1ccc(-c2ccc3c(oc4ccccc43)c2N(c2ccc(-c3cccc4ccccc34)cc2)c2ccc3sc4ccccc4c3c2)cc1 ; c1ccc(-c2ccc3c(oc4ccccc43)c2N(c2ccc(-c3ccc4ccccc4c3)cc2)c2ccc3sc4ccccc4c3c2)cc1 ; c1ccc(-c2ccccc2-c2ccc3c(oc4ccccc43)c2N(c2ccc(-c3ccc4ccccc4c3)cc2)c2ccc3sc4ccccc4c3c2)cc1] 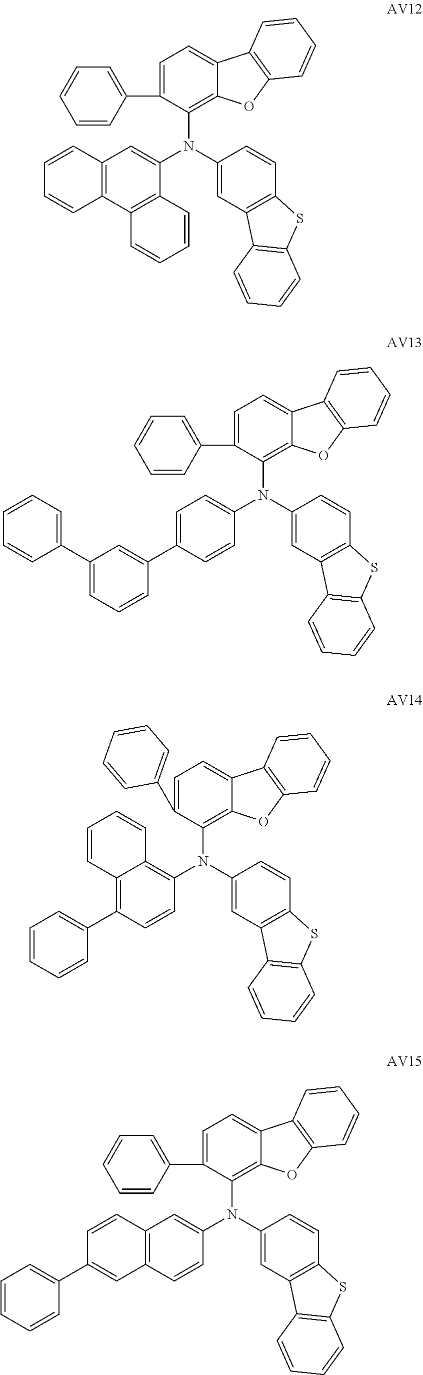 [SMILES: c1ccc(-c2ccc3c(oc4ccccc43)c2N(c2ccc3sc4ccccc4c3c2)c2cc3ccccc3c3ccccc23)cc1 ; c1ccc(-c2cccc(-c3ccc(N(c4ccc5sc6ccccc6c5c4)c4c(-c5ccccc5)ccc5c4oc4ccccc45)cc3)c2)cc1 ; c1ccc(-c2ccc3c(oc4ccccc43)c2N(c2ccc3sc4ccccc4c3c2)c2ccc(-c3ccccc3)c3ccccc23)cc1 ; c1ccc(-c2ccc3cc(N(c4ccc5sc6ccccc6c5c4)c4c(-c5ccccc5)ccc5c4oc4ccccc45)ccc3c2)cc1] 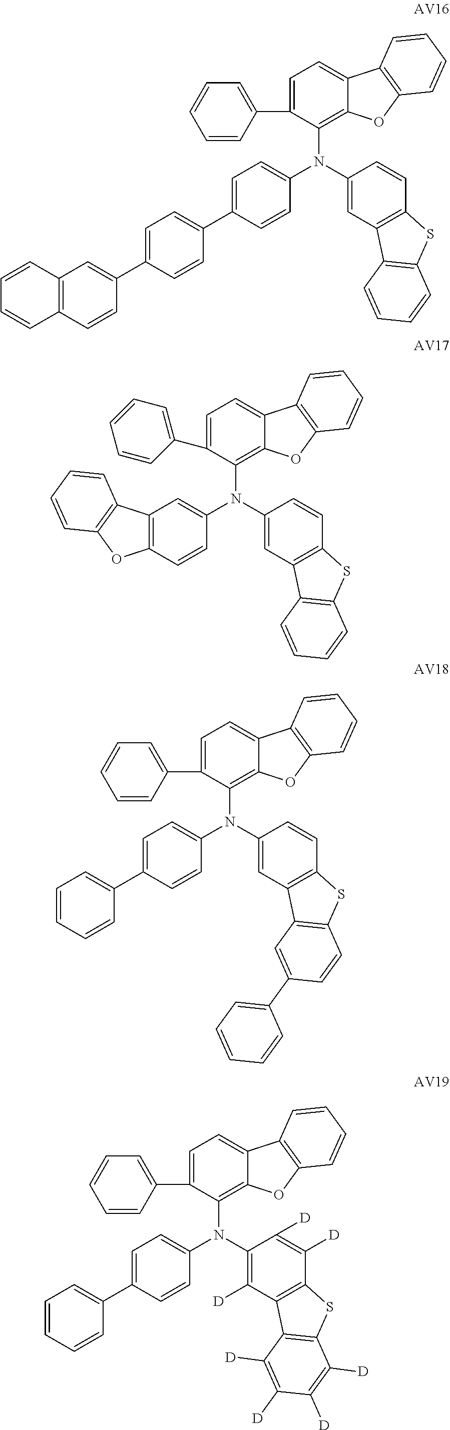 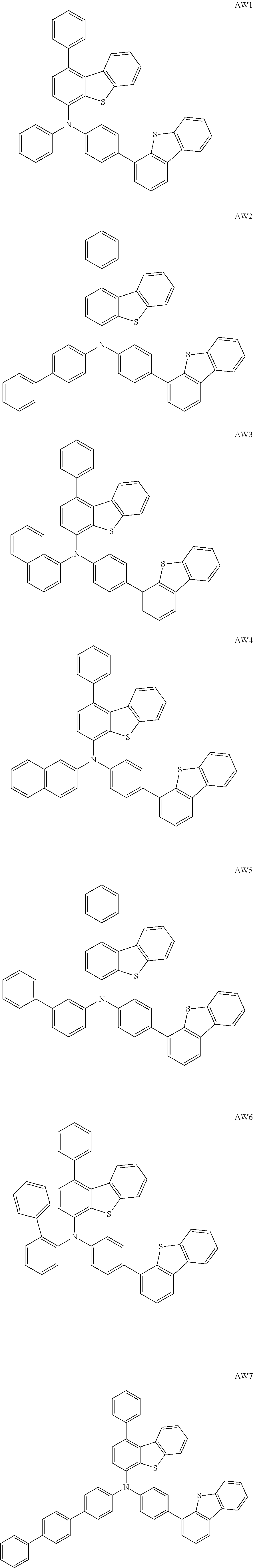 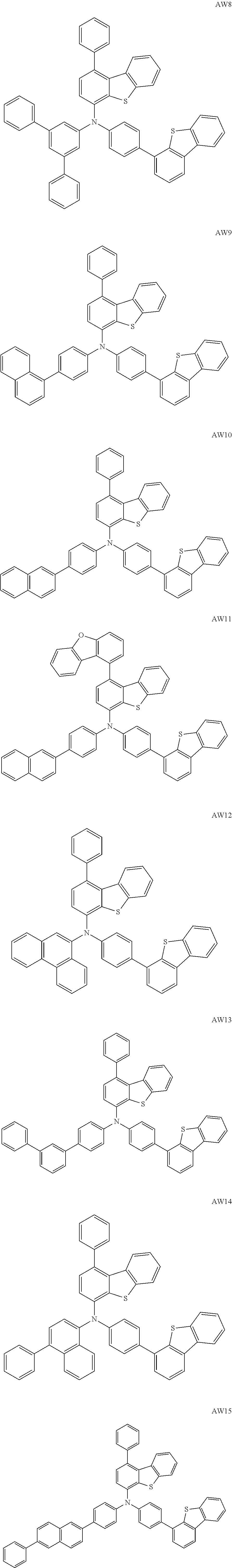 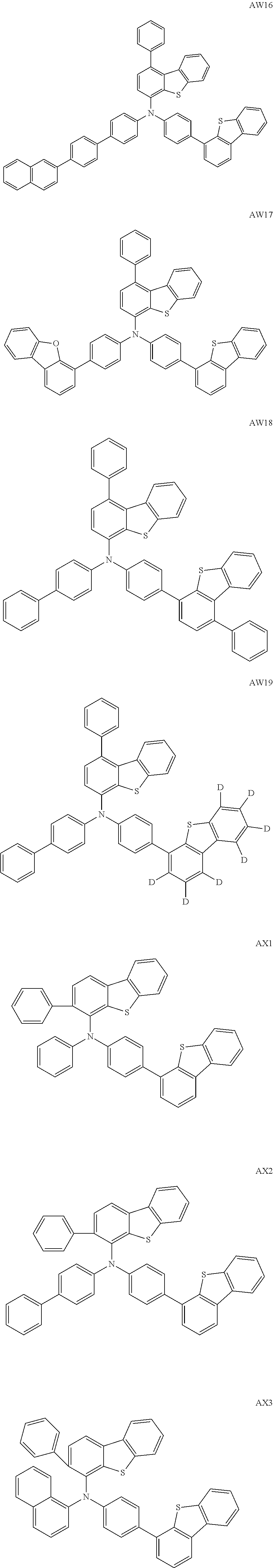 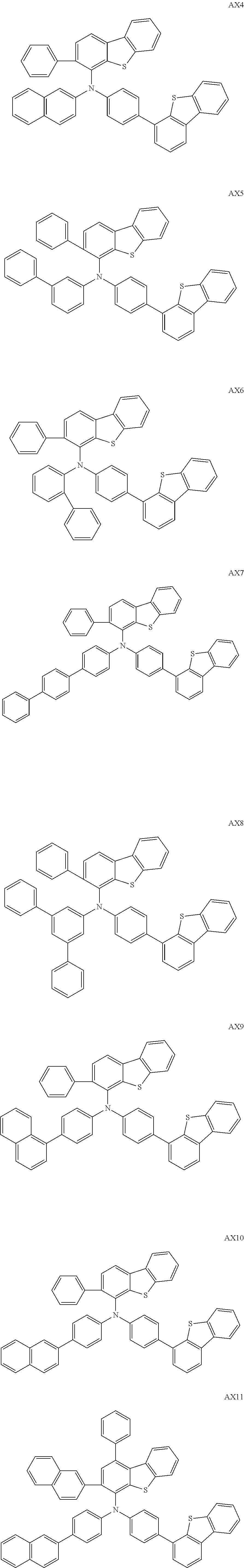 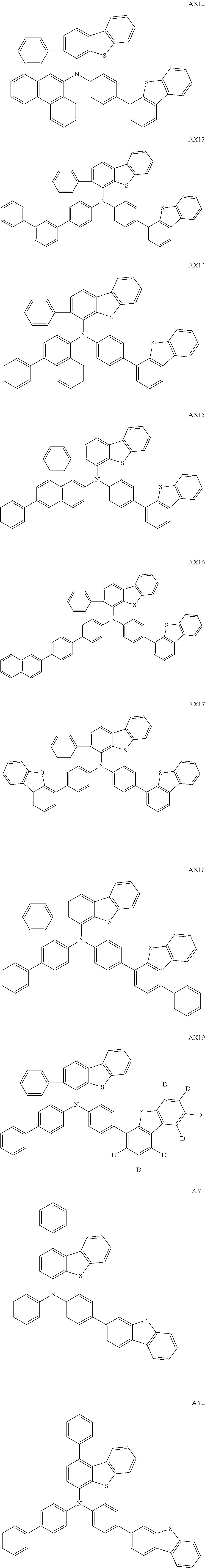 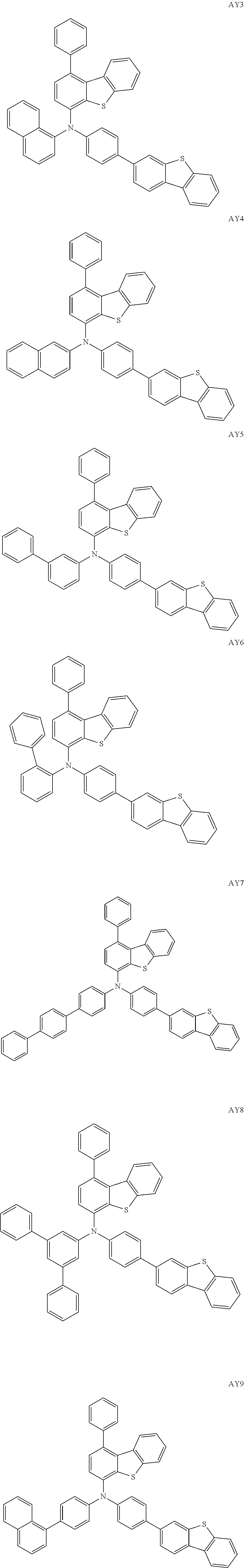 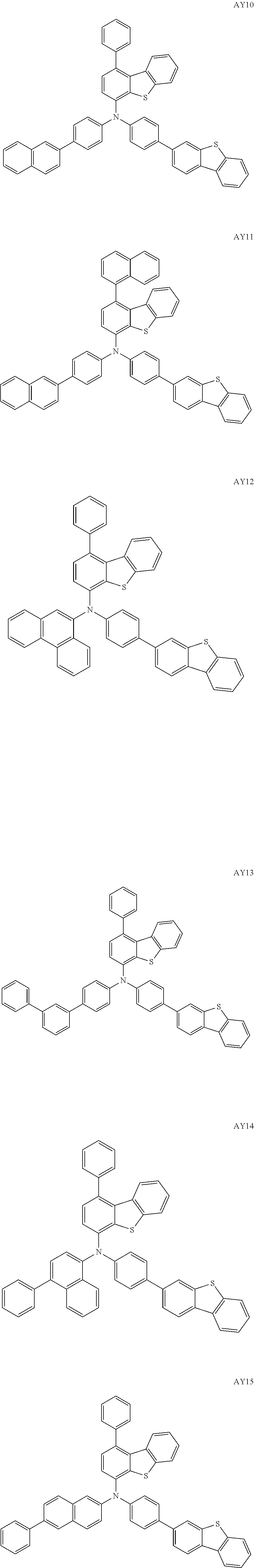 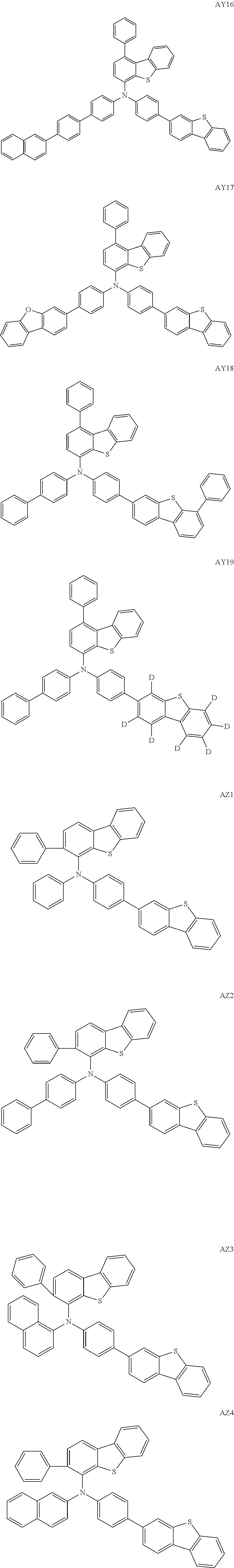 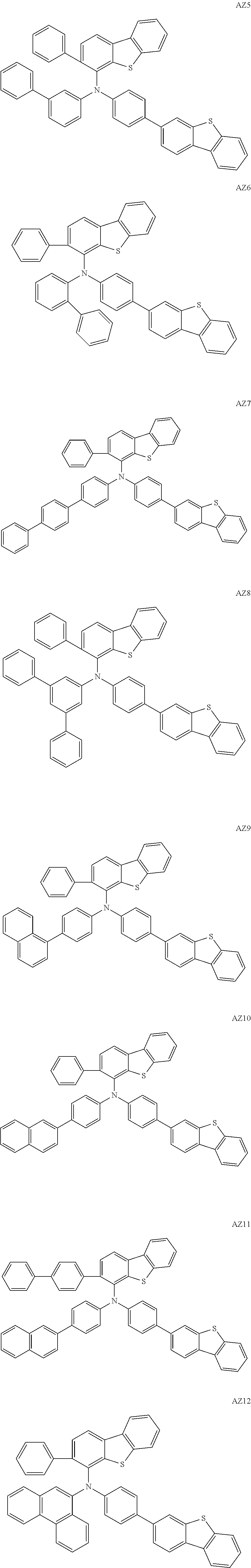 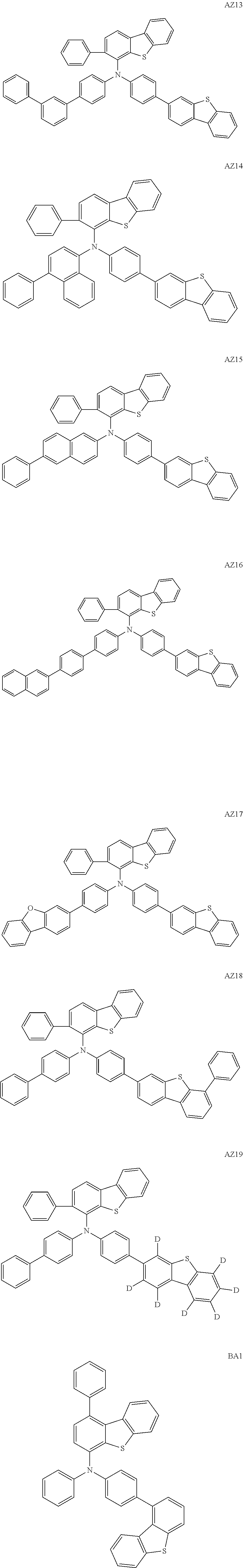 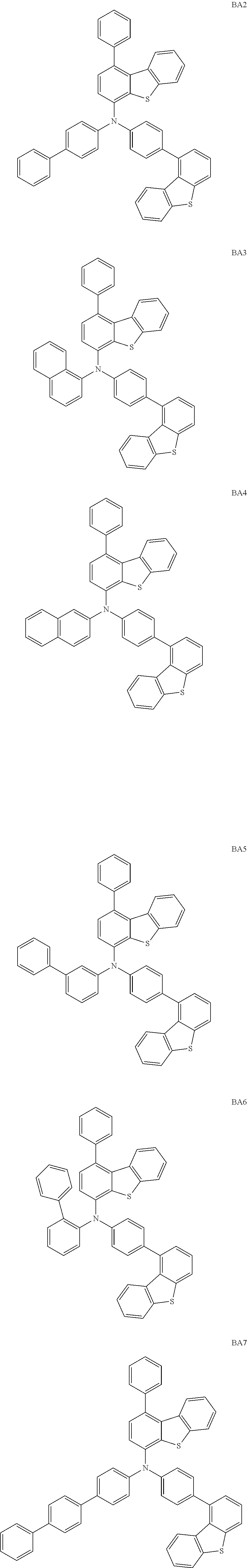 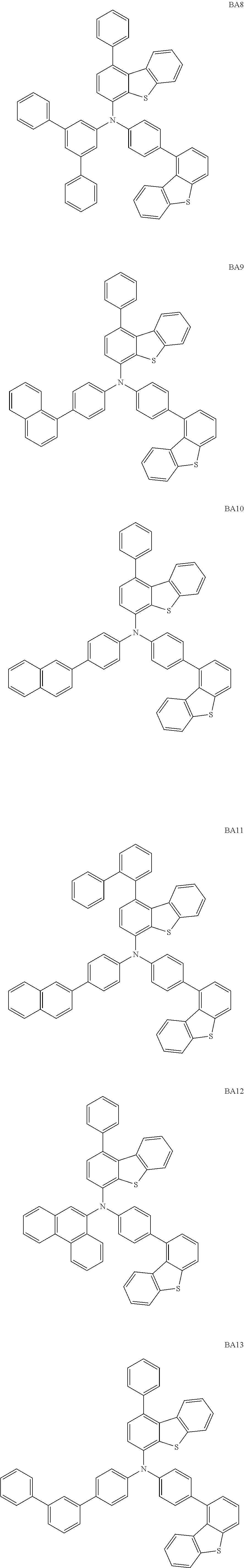 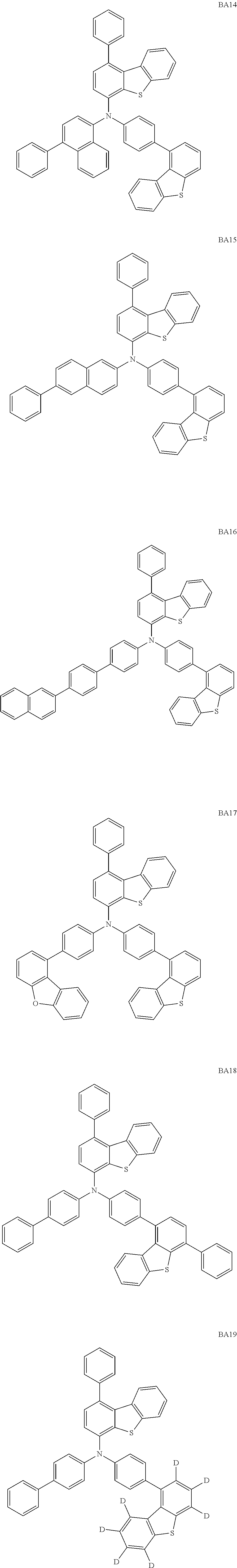 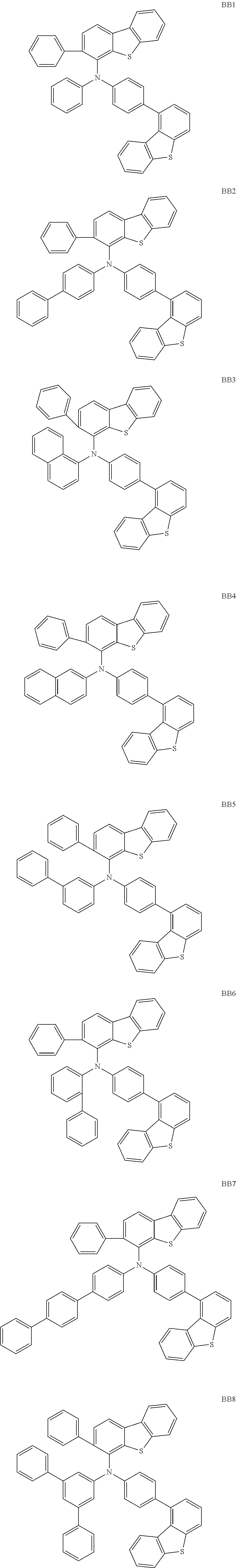 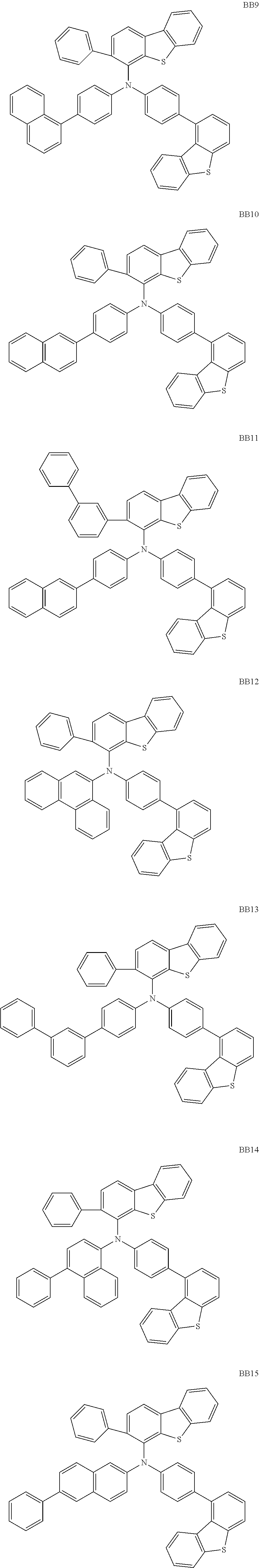 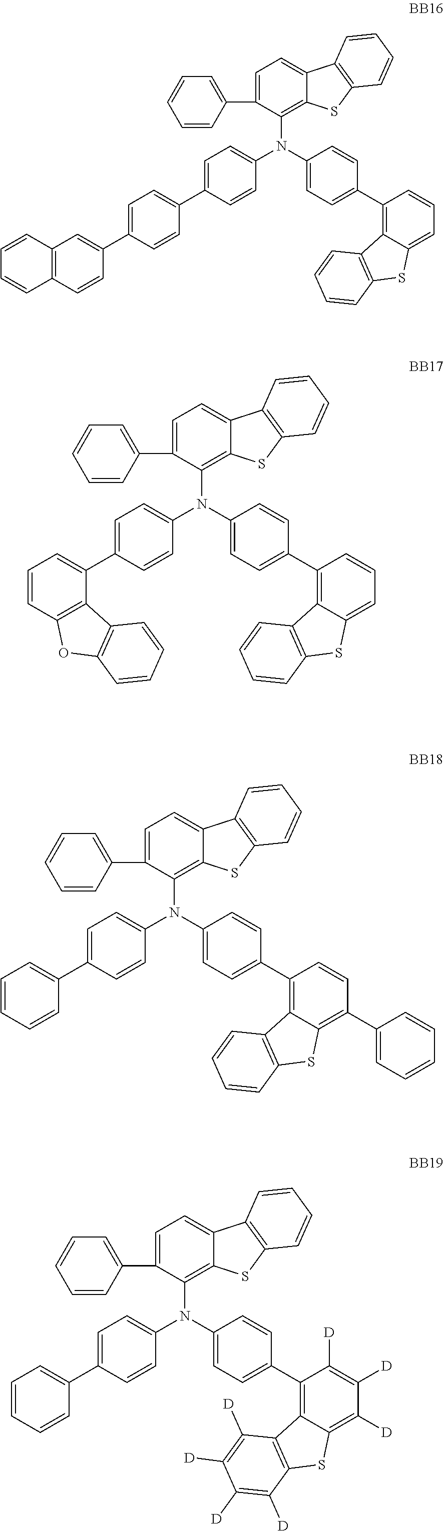 [SMILES: c1ccc(-c2ccc3c(sc4ccccc43)c2N(c2ccc(-c3ccc(-c4ccc5ccccc5c4)cc3)cc2)c2ccc(-c3cccc4sc5ccccc5c34)cc2)cc1 ; c1ccc(-c2ccc3c(sc4ccccc43)c2N(c2ccc(-c3cccc4oc5ccccc5c34)cc2)c2ccc(-c3cccc4sc5ccccc5c34)cc2)cc1 ; c1ccc(-c2ccc(N(c3ccc(-c4ccc(-c5ccccc5)c5sc6ccccc6c45)cc3)c3c(-c4ccccc4)ccc4c3sc3ccccc34)cc2)cc1 ; [2H]c1c([2H])c([2H])c2c(sc3c([2H])c([2H])c([2H])c(-c4ccc(N(c5ccc(-c6ccccc6)cc5)c5c(-c6ccccc6)ccc6c5sc5ccccc56)cc4)c32)c1[2H]] 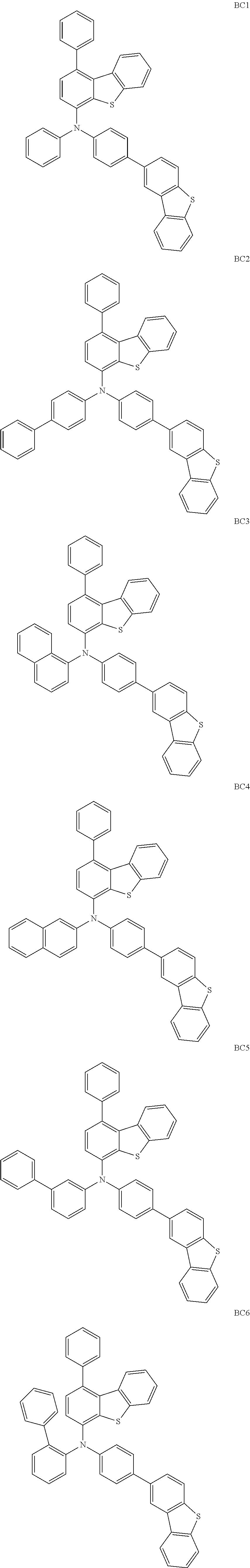 [SMILES: c1ccc(-c2ccc(N(c3ccccc3)c3ccc(-c4ccc5sc6ccccc6c5c4)cc3)c3sc4ccccc4c23)cc1 ; c1ccc(-c2ccc(N(c3ccc(-c4ccc5sc6ccccc6c5c4)cc3)c3ccc(-c4ccccc4)c4c3sc3ccccc34)cc2)cc1 ; c1ccc(-c2ccc(N(c3ccc(-c4ccc5sc6ccccc6c5c4)cc3)c3cccc4ccccc34)c3sc4ccccc4c23)cc1 ; c1ccc(-c2ccc(N(c3ccc(-c4ccc5sc6ccccc6c5c4)cc3)c3ccc4ccccc4c3)c3sc4ccccc4c23)cc1 ; c1ccc(-c2cccc(N(c3ccc(-c4ccc5sc6ccccc6c5c4)cc3)c3ccc(-c4ccccc4)c4c3sc3ccccc34)c2)cc1 ; c1ccc(-c2ccccc2N(c2ccc(-c3ccc4sc5ccccc5c4c3)cc2)c2ccc(-c3ccccc3)c3c2sc2ccccc23)cc1] 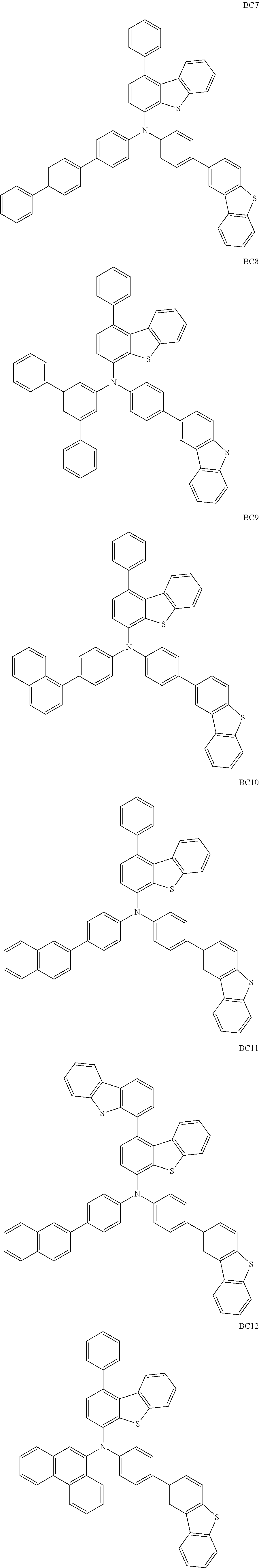 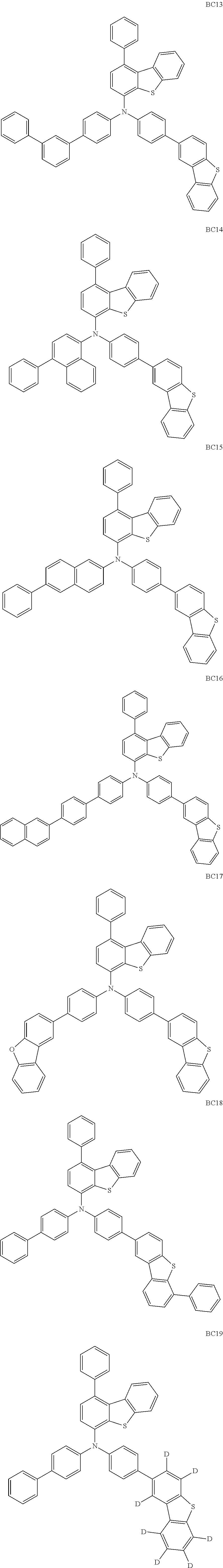 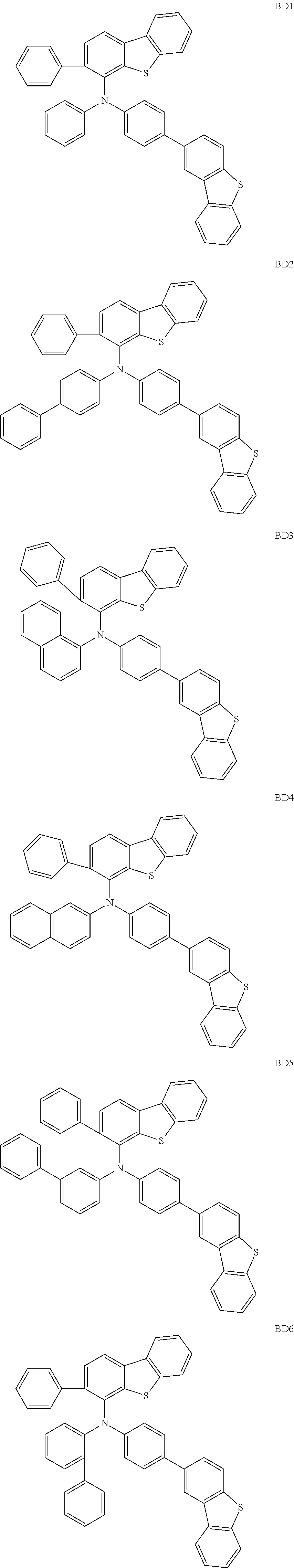 [SMILES: c1ccc(-c2ccc3c(sc4ccccc43)c2N(c2ccccc2)c2ccc(-c3ccc4sc5ccccc5c4c3)cc2)cc1 ; c1ccc(-c2ccc(N(c3ccc(-c4ccc5sc6ccccc6c5c4)cc3)c3c(-c4ccccc4)ccc4c3sc3ccccc34)cc2)cc1 ; c1ccc(-c2ccc3c(sc4ccccc43)c2N(c2ccc(-c3ccc4sc5ccccc5c4c3)cc2)c2cccc3ccccc23)cc1 ; c1ccc(-c2ccc3c(sc4ccccc43)c2N(c2ccc(-c3ccc4sc5ccccc5c4c3)cc2)c2ccc3ccccc3c2)cc1 ; c1ccc(-c2cccc(N(c3ccc(-c4ccc5sc6ccccc6c5c4)cc3)c3c(-c4ccccc4)ccc4c3sc3ccccc34)c2)cc1 ; c1ccc(-c2ccccc2N(c2ccc(-c3ccc4sc5ccccc5c4c3)cc2)c2c(-c3ccccc3)ccc3c2sc2ccccc23)cc1] 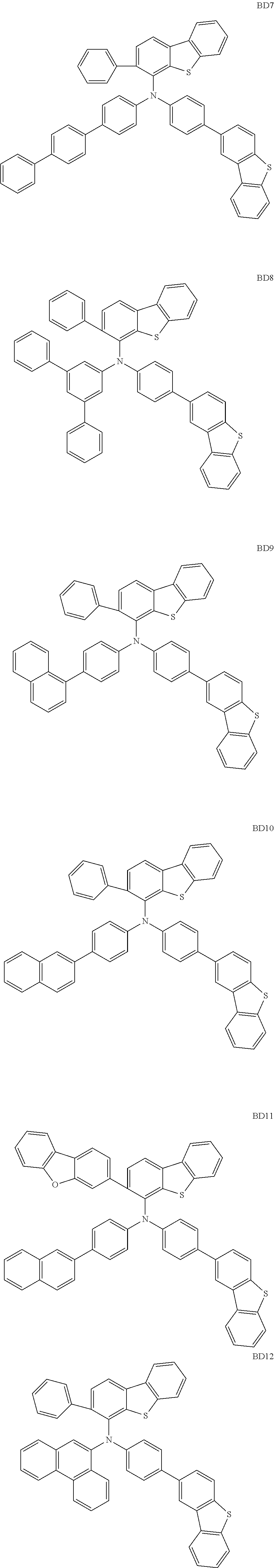 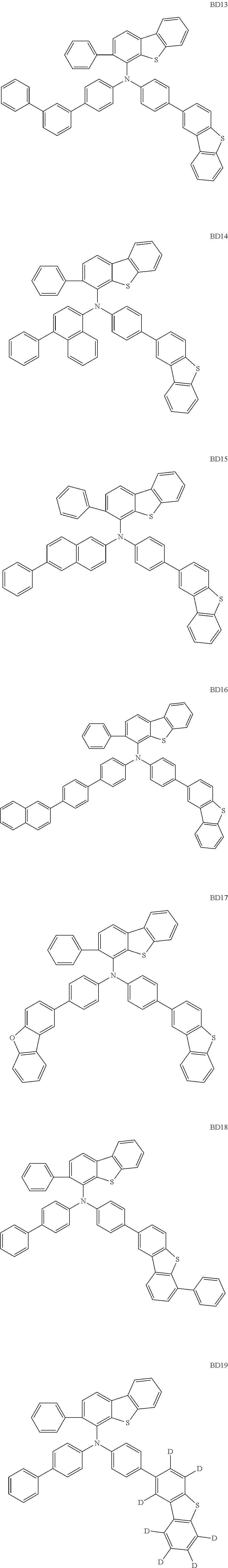 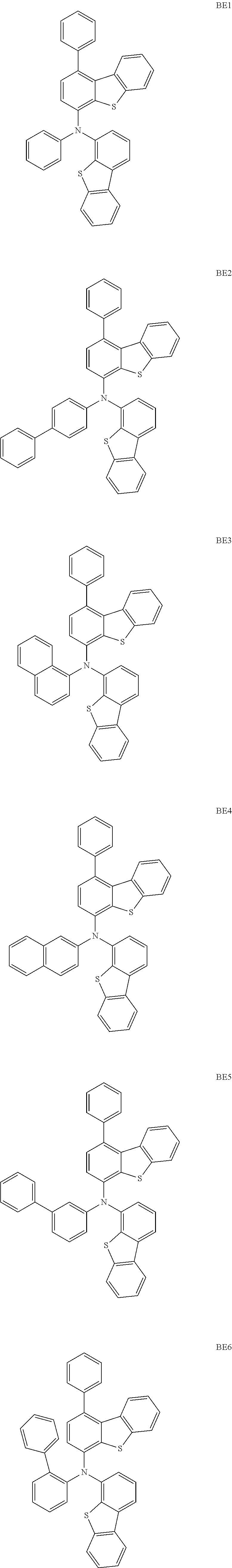 [SMILES: c1ccc(-c2ccc(N(c3ccccc3)c3cccc4c3sc3ccccc34)c3sc4ccccc4c23)cc1 ; c1ccc(-c2ccc(N(c3cccc4c3sc3ccccc34)c3ccc(-c4ccccc4)c4c3sc3ccccc34)cc2)cc1 ; c1ccc(-c2ccc(N(c3cccc4ccccc34)c3cccc4c3sc3ccccc34)c3sc4ccccc4c23)cc1 ; c1ccc(-c2ccc(N(c3ccc4ccccc4c3)c3cccc4c3sc3ccccc34)c3sc4ccccc4c23)cc1 ; c1ccc(-c2cccc(N(c3cccc4c3sc3ccccc34)c3ccc(-c4ccccc4)c4c3sc3ccccc34)c2)cc1 ; c1ccc(-c2ccccc2N(c2cccc3c2sc2ccccc23)c2ccc(-c3ccccc3)c3c2sc2ccccc23)cc1] 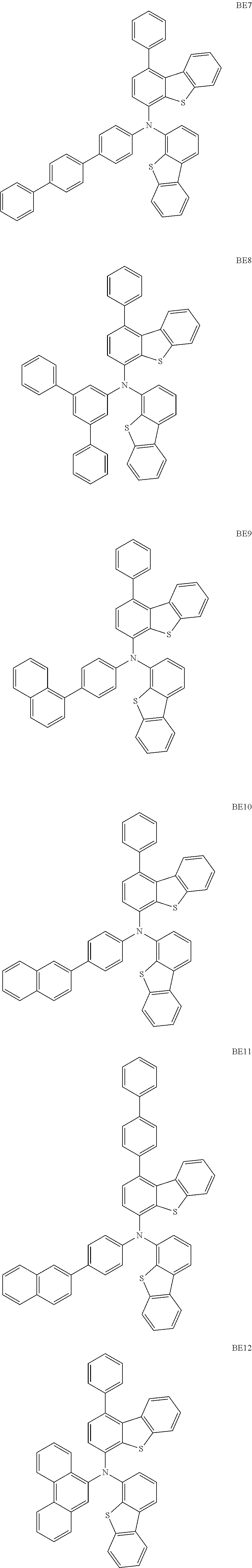 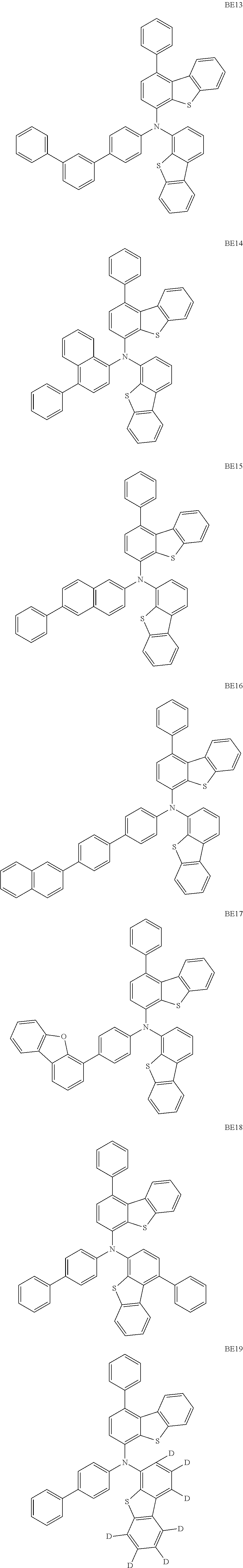 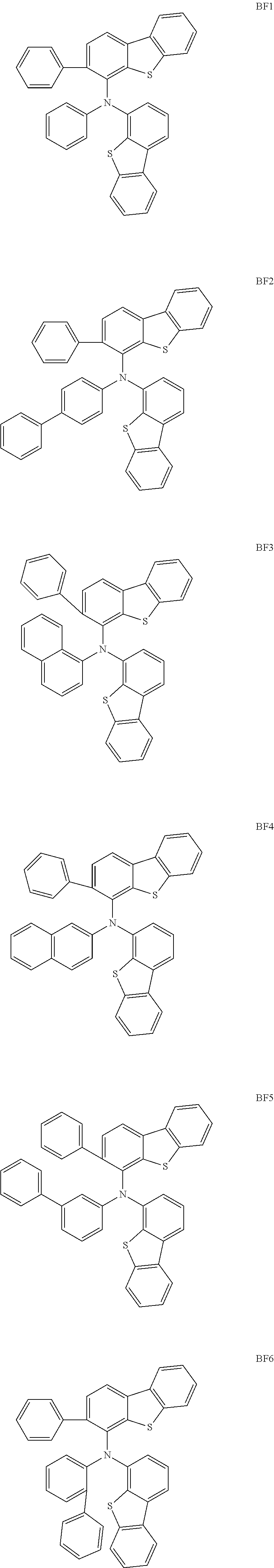 [SMILES: c1ccc(-c2ccc3c(sc4ccccc43)c2N(c2ccccc2)c2cccc3c2sc2ccccc23)cc1 ; c1ccc(-c2ccc(N(c3cccc4c3sc3ccccc34)c3c(-c4ccccc4)ccc4c3sc3ccccc34)cc2)cc1 ; c1ccc(-c2ccc3c(sc4ccccc43)c2N(c2cccc3ccccc23)c2cccc3c2sc2ccccc23)cc1 ; c1ccc(-c2ccc3c(sc4ccccc43)c2N(c2ccc3ccccc3c2)c2cccc3c2sc2ccccc23)cc1 ; c1ccc(-c2cccc(N(c3cccc4c3sc3ccccc34)c3c(-c4ccccc4)ccc4c3sc3ccccc34)c2)cc1 ; c1ccc(-c2ccccc2N(c2cccc3c2sc2ccccc23)c2c(-c3ccccc3)ccc3c2sc2ccccc23)cc1] 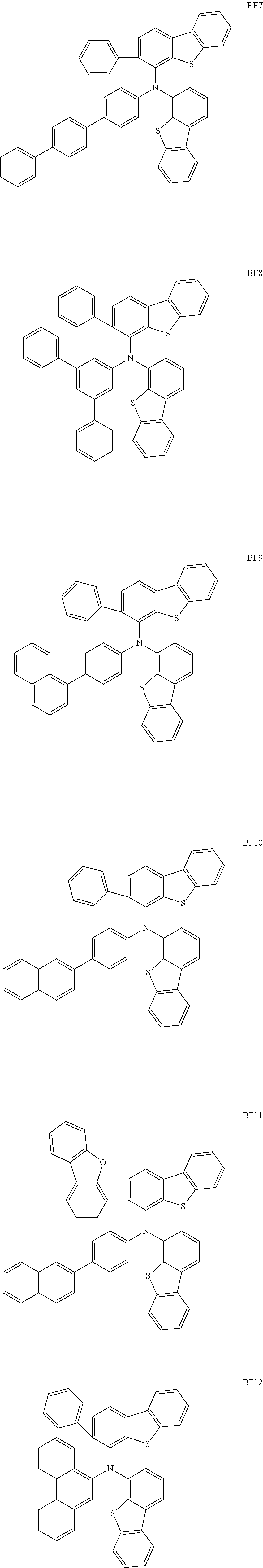 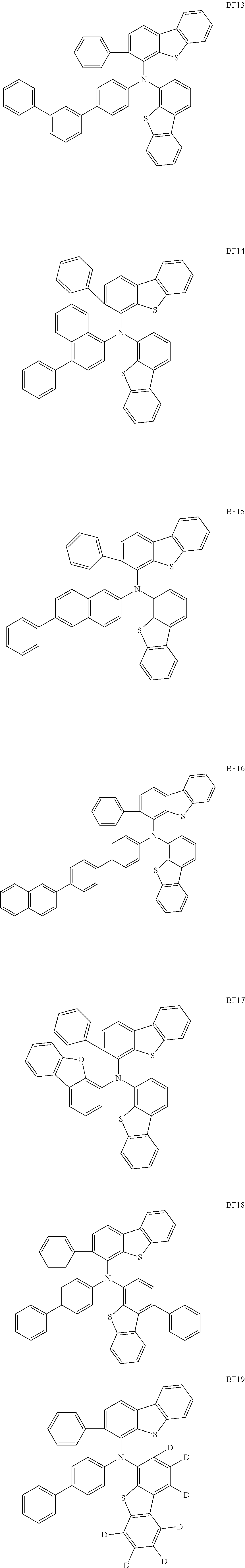 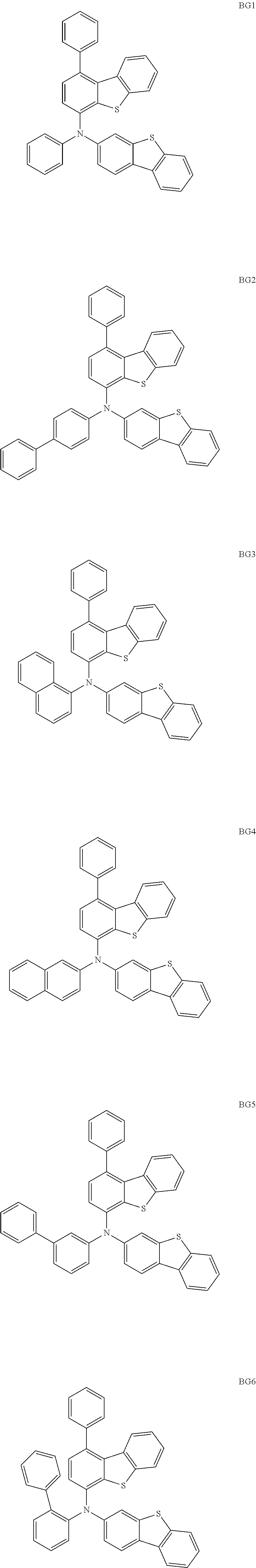 [SMILES: c1ccc(-c2ccc(N(c3ccccc3)c3ccc4c(c3)sc3ccccc34)c3sc4ccccc4c23)cc1 ; c1ccc(-c2ccc(N(c3ccc4c(c3)sc3ccccc34)c3ccc(-c4ccccc4)c4c3sc3ccccc34)cc2)cc1 ; c1ccc(-c2ccc(N(c3ccc4c(c3)sc3ccccc34)c3cccc4ccccc34)c3sc4ccccc4c23)cc1 ; c1ccc(-c2ccc(N(c3ccc4ccccc4c3)c3ccc4c(c3)sc3ccccc34)c3sc4ccccc4c23)cc1 ; c1ccc(-c2cccc(N(c3ccc4c(c3)sc3ccccc34)c3ccc(-c4ccccc4)c4c3sc3ccccc34)c2)cc1 ; c1ccc(-c2ccccc2N(c2ccc3c(c2)sc2ccccc23)c2ccc(-c3ccccc3)c3c2sc2ccccc23)cc1] 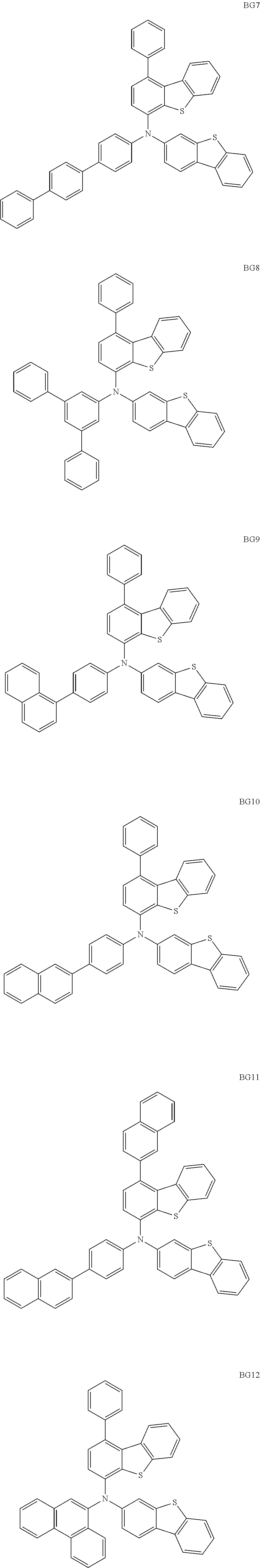 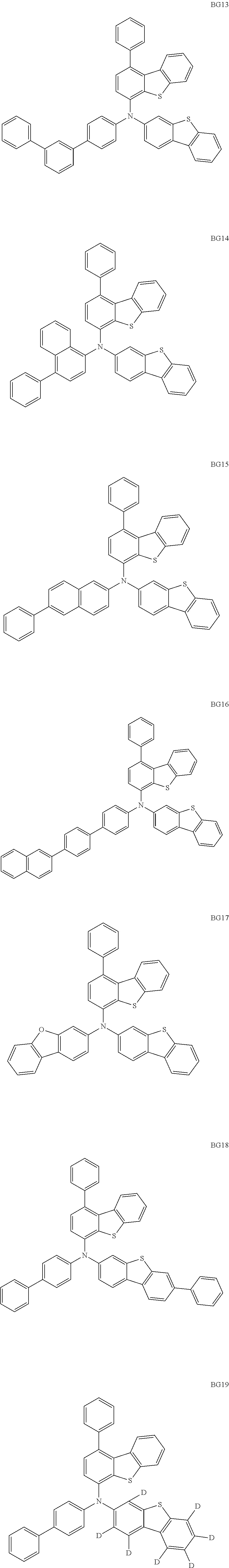 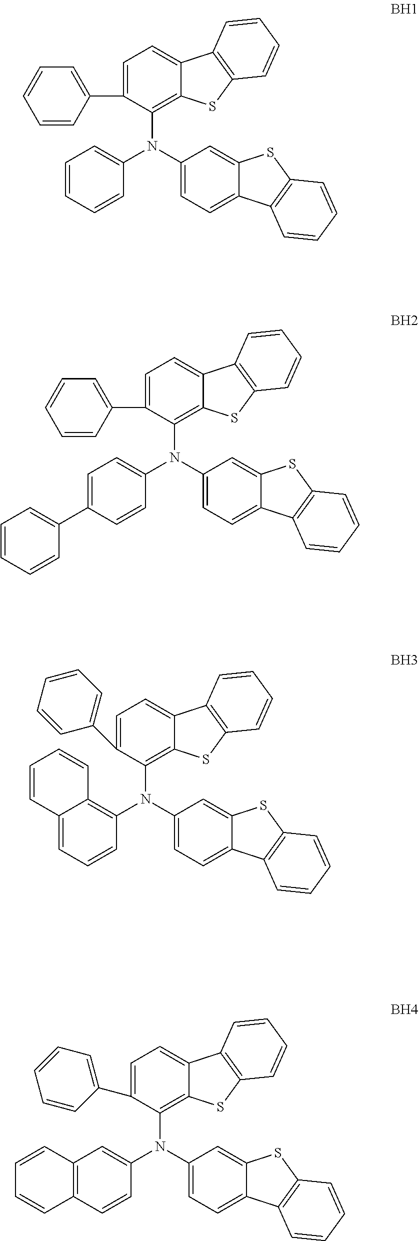 [SMILES: c1ccc(-c2ccc3c(sc4ccccc43)c2N(c2ccccc2)c2ccc3c(c2)sc2ccccc23)cc1 ; c1ccc(-c2ccc(N(c3ccc4c(c3)sc3ccccc34)c3c(-c4ccccc4)ccc4c3sc3ccccc34)cc2)cc1 ; c1ccc(-c2ccc3c(sc4ccccc43)c2N(c2ccc3c(c2)sc2ccccc23)c2cccc3ccccc23)cc1 ; c1ccc(-c2ccc3c(sc4ccccc43)c2N(c2ccc3ccccc3c2)c2ccc3c(c2)sc2ccccc23)cc1] 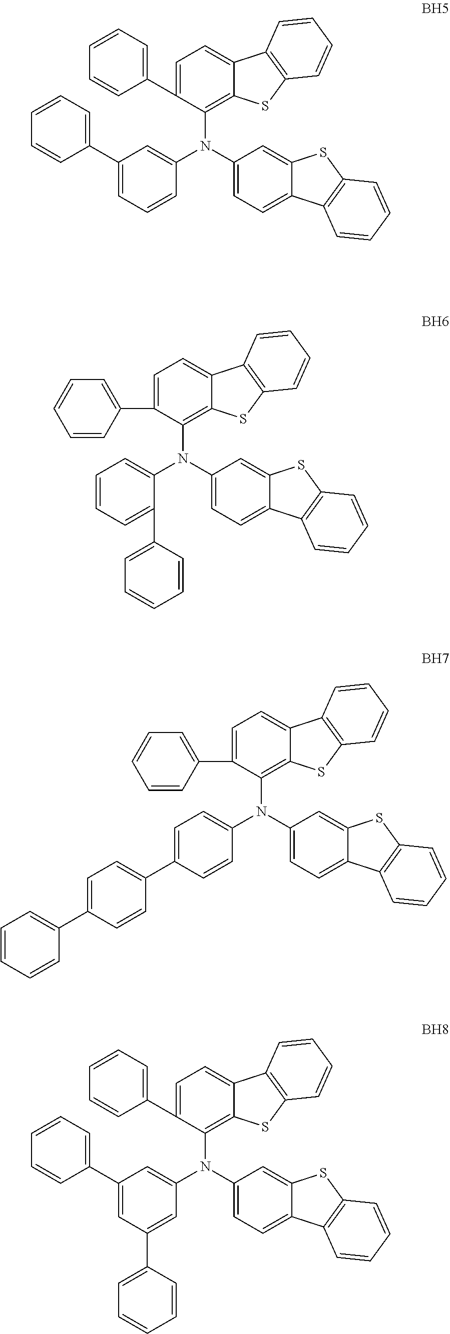 [SMILES: c1ccc(-c2cccc(N(c3ccc4c(c3)sc3ccccc34)c3c(-c4ccccc4)ccc4c3sc3ccccc34)c2)cc1 ; c1ccc(-c2ccccc2N(c2ccc3c(c2)sc2ccccc23)c2c(-c3ccccc3)ccc3c2sc2ccccc23)cc1 ; c1ccc(-c2ccc(-c3ccc(N(c4ccc5c(c4)sc4ccccc45)c4c(-c5ccccc5)ccc5c4sc4ccccc45)cc3)cc2)cc1 ; c1ccc(-c2cc(-c3ccccc3)cc(N(c3ccc4c(c3)sc3ccccc34)c3c(-c4ccccc4)ccc4c3sc3ccccc34)c2)cc1] 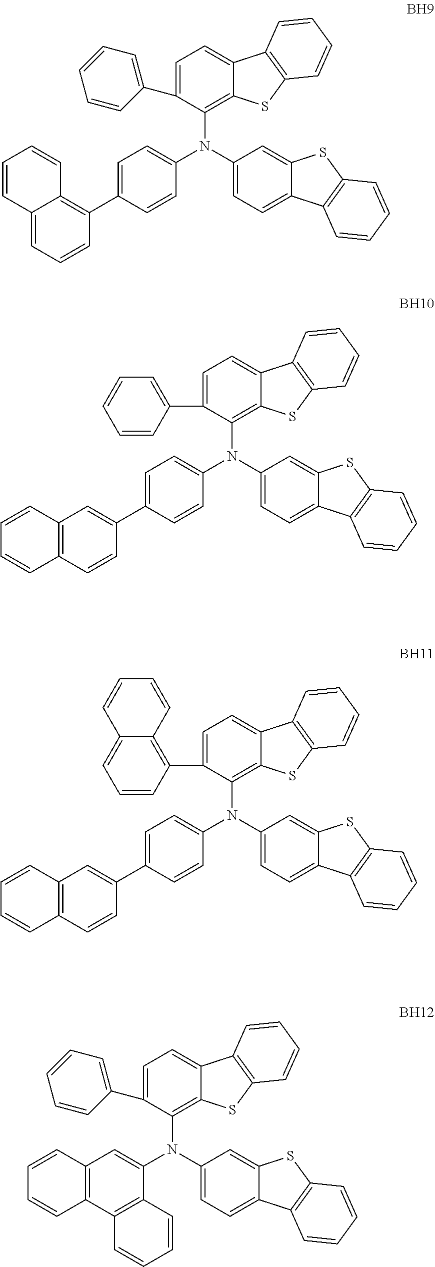 [SMILES: c1ccc(-c2ccc3c(sc4ccccc43)c2N(c2ccc(-c3cccc4ccccc34)cc2)c2ccc3c(c2)sc2ccccc23)cc1 ; c1ccc(-c2ccc3c(sc4ccccc43)c2N(c2ccc(-c3ccc4ccccc4c3)cc2)c2ccc3c(c2)sc2ccccc23)cc1 ; c1ccc2cc(-c3ccc(N(c4ccc5c(c4)sc4ccccc45)c4c(-c5cccc6ccccc56)ccc5c4sc4ccccc45)cc3)ccc2c1 ; c1ccc(-c2ccc3c(sc4ccccc43)c2N(c2ccc3c(c2)sc2ccccc23)c2cc3ccccc3c3ccccc23)cc1] 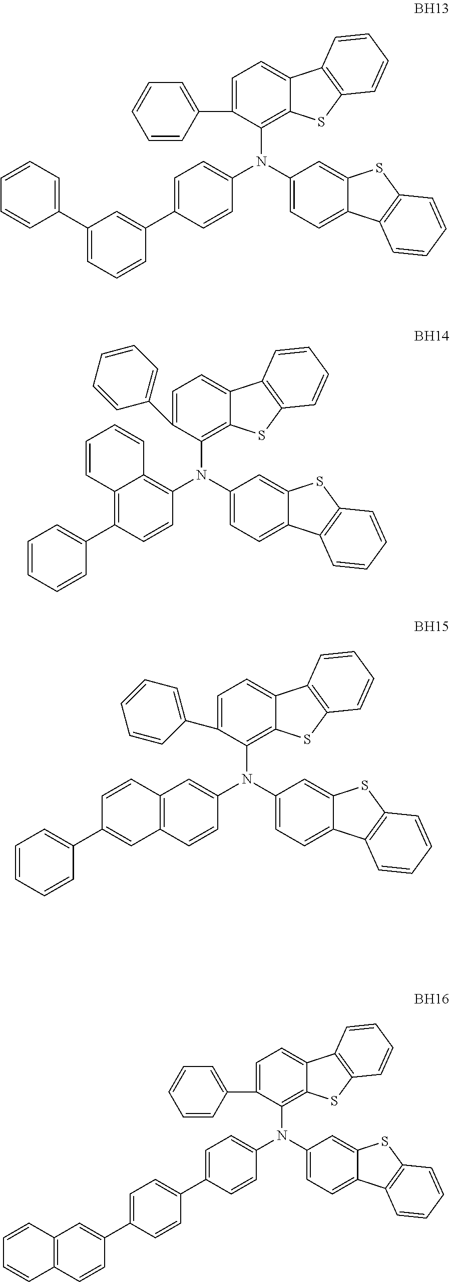 [SMILES: c1ccc(-c2cccc(-c3ccc(N(c4ccc5c(c4)sc4ccccc45)c4c(-c5ccccc5)ccc5c4sc4ccccc45)cc3)c2)cc1 ; c1ccc(-c2ccc3c(sc4ccccc43)c2N(c2ccc3c(c2)sc2ccccc23)c2ccc(-c3ccccc3)c3ccccc23)cc1 ; c1ccc(-c2ccc3cc(N(c4ccc5c(c4)sc4ccccc45)c4c(-c5ccccc5)ccc5c4sc4ccccc45)ccc3c2)cc1 ; c1ccc(-c2ccc3c(sc4ccccc43)c2N(c2ccc(-c3ccc(-c4ccc5ccccc5c4)cc3)cc2)c2ccc3c(c2)sc2ccccc23)cc1] 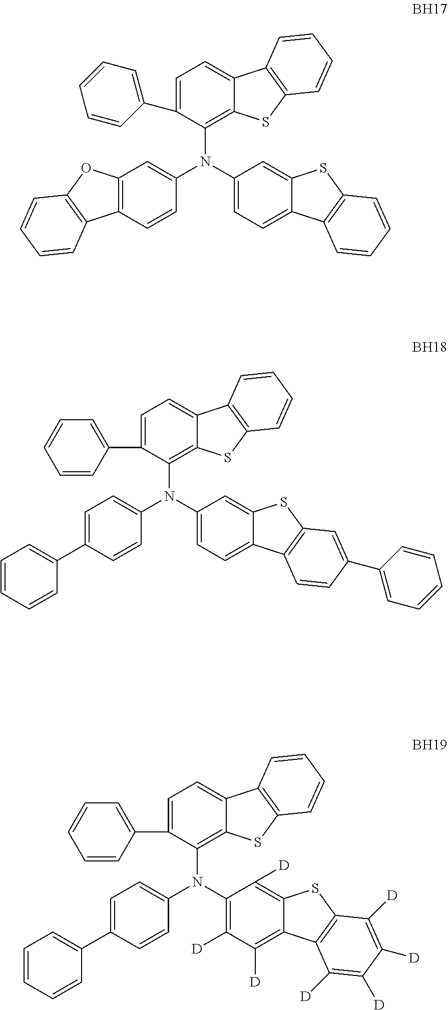 [SMILES: c1ccc(-c2ccc3c(sc4ccccc43)c2N(c2ccc3c(c2)oc2ccccc23)c2ccc3c(c2)sc2ccccc23)cc1 ; c1ccc(-c2ccc(N(c3ccc4c(c3)sc3cc(-c5ccccc5)ccc34)c3c(-c4ccccc4)ccc4c3sc3ccccc34)cc2)cc1 ; [2H]c1c([2H])c([2H])c2c(sc3c([2H])c(N(c4ccc(-c5ccccc5)cc4)c4c(-c5ccccc5)ccc5c4sc4ccccc45)c([2H])c([2H])c32)c1[2H]] 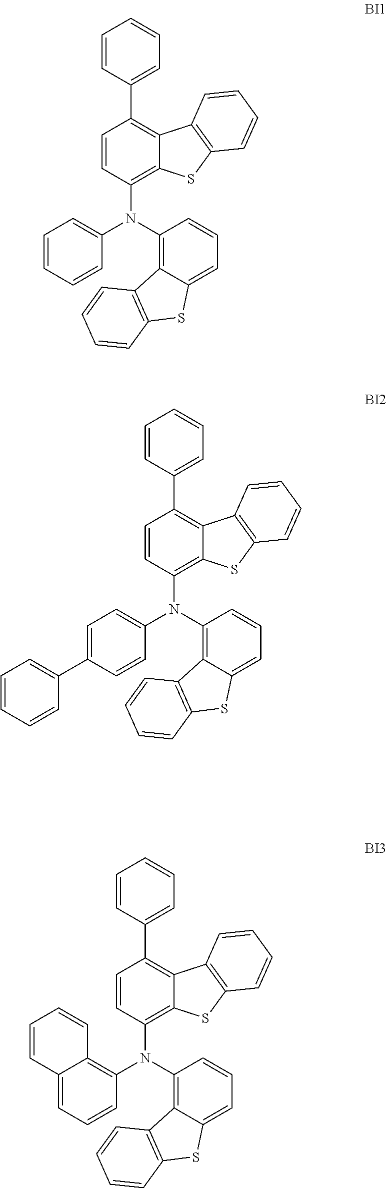 [SMILES: c1ccc(-c2ccc(N(c3ccccc3)c3cccc4sc5ccccc5c34)c3sc4ccccc4c23)cc1 ; c1ccc(-c2ccc(N(c3ccc(-c4ccccc4)c4c3sc3ccccc34)c3cccc4sc5ccccc5c34)cc2)cc1 ; c1ccc(-c2ccc(N(c3cccc4ccccc34)c3cccc4sc5ccccc5c34)c3sc4ccccc4c23)cc1] 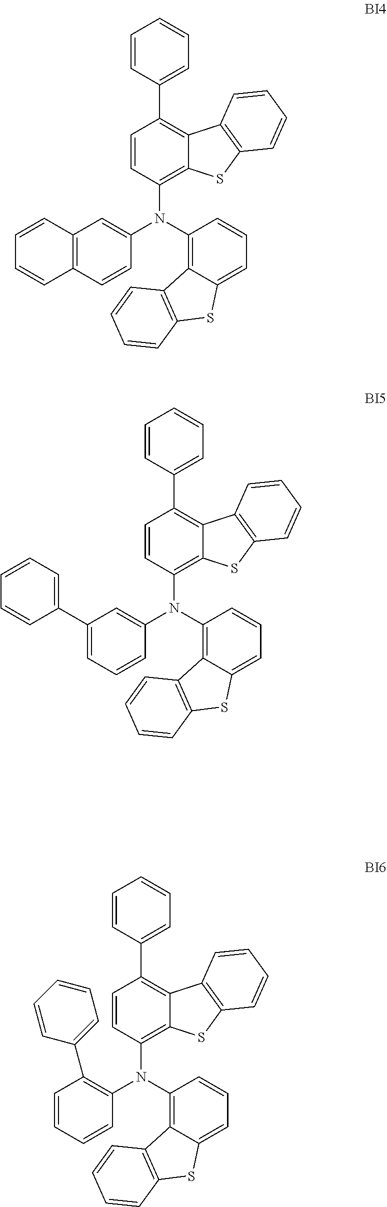 [SMILES: c1ccc(-c2ccc(N(c3ccc4ccccc4c3)c3cccc4sc5ccccc5c34)c3sc4ccccc4c23)cc1 ; c1ccc(-c2cccc(N(c3ccc(-c4ccccc4)c4c3sc3ccccc34)c3cccc4sc5ccccc5c34)c2)cc1 ; c1ccc(-c2ccccc2N(c2ccc(-c3ccccc3)c3c2sc2ccccc23)c2cccc3sc4ccccc4c23)cc1] 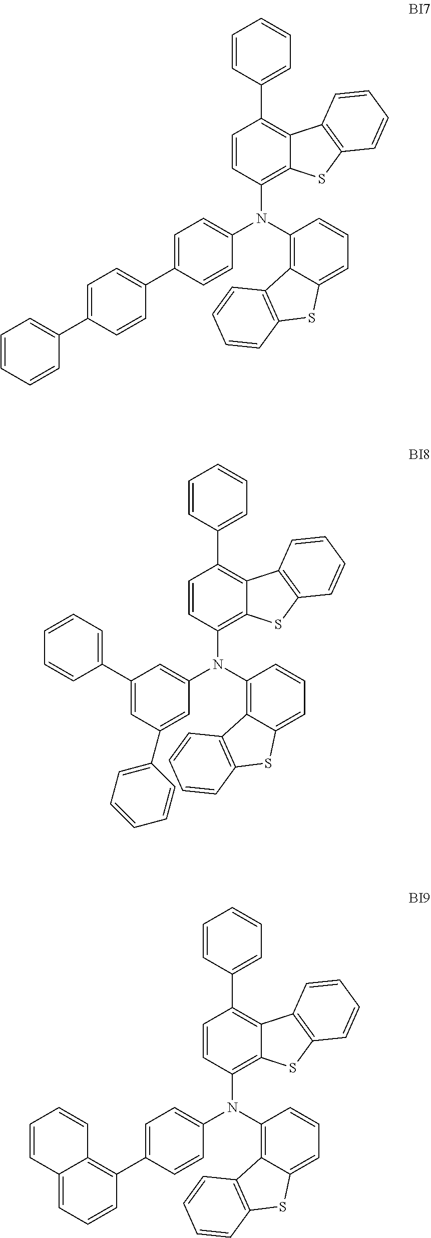 [SMILES: c1ccc(-c2ccc(-c3ccc(N(c4ccc(-c5ccccc5)c5c4sc4ccccc45)c4cccc5sc6ccccc6c45)cc3)cc2)cc1 ; c1ccc(-c2cc(-c3ccccc3)cc(N(c3ccc(-c4ccccc4)c4c3sc3ccccc34)c3cccc4sc5ccccc5c34)c2)cc1 ; c1ccc(-c2ccc(N(c3ccc(-c4cccc5ccccc45)cc3)c3cccc4sc5ccccc5c34)c3sc4ccccc4c23)cc1] 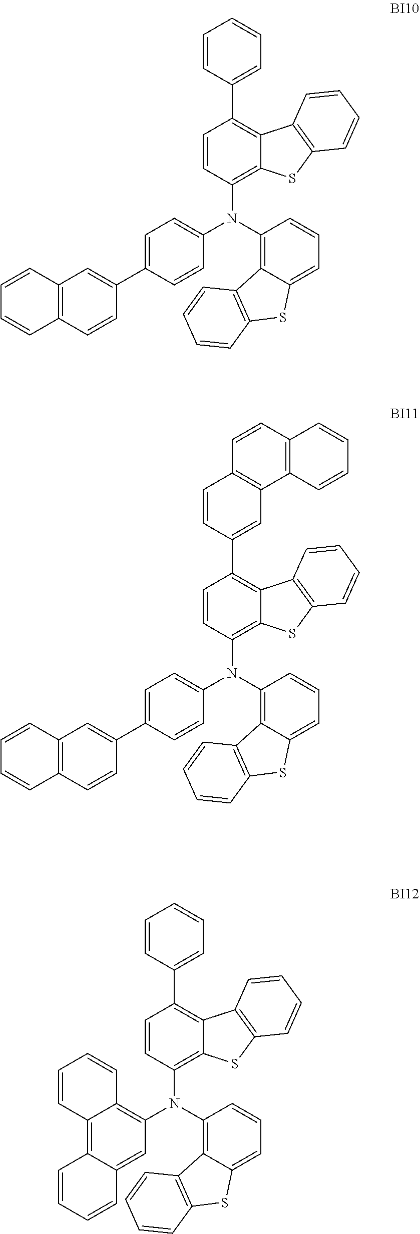 [SMILES: c1ccc(-c2ccc(N(c3ccc(-c4ccc5ccccc5c4)cc3)c3cccc4sc5ccccc5c34)c3sc4ccccc4c23)cc1 ; c1ccc2cc(-c3ccc(N(c4ccc(-c5ccc6ccc7ccccc7c6c5)c5c4sc4ccccc45)c4cccc5sc6ccccc6c45)cc3)ccc2c1 ; c1ccc(-c2ccc(N(c3cc4ccccc4c4ccccc34)c3cccc4sc5ccccc5c34)c3sc4ccccc4c23)cc1] 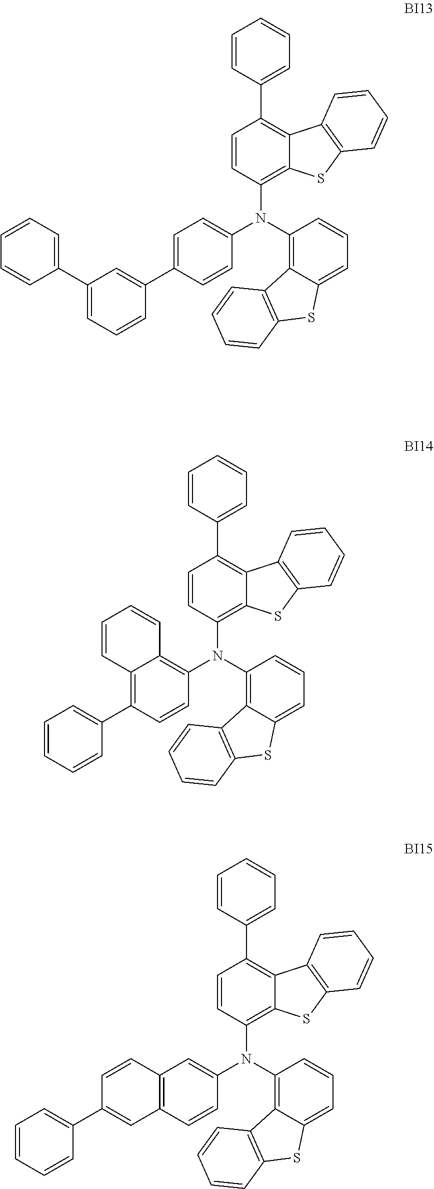 [SMILES: c1ccc(-c2cccc(-c3ccc(N(c4ccc(-c5ccccc5)c5c4sc4ccccc45)c4cccc5sc6ccccc6c45)cc3)c2)cc1 ; c1ccc(-c2ccc(N(c3ccc(-c4ccccc4)c4c3sc3ccccc34)c3cccc4sc5ccccc5c34)c3ccccc23)cc1 ; c1ccc(-c2ccc3cc(N(c4ccc(-c5ccccc5)c5c4sc4ccccc45)c4cccc5sc6ccccc6c45)ccc3c2)cc1] 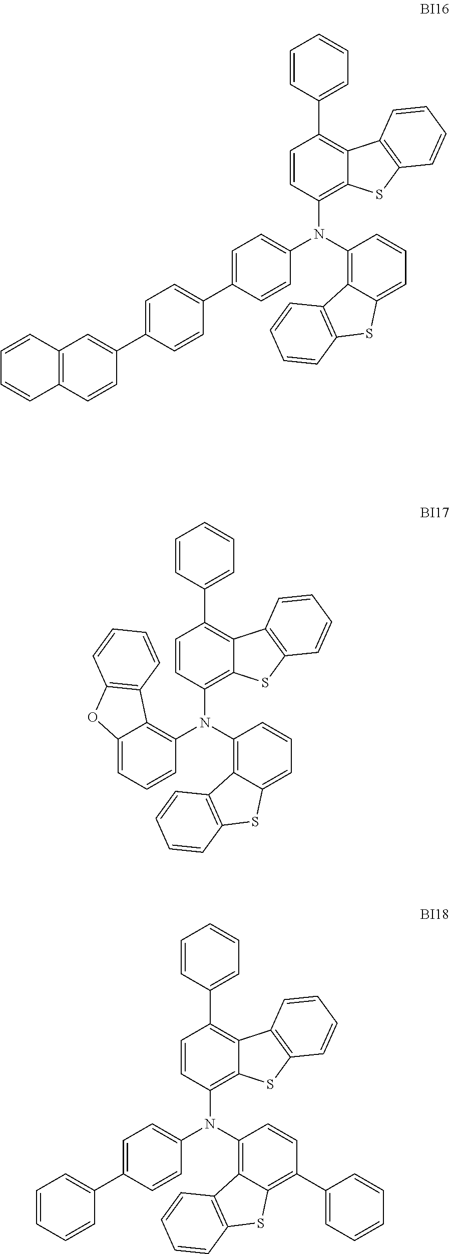 [SMILES: c1ccc(-c2ccc(N(c3ccc(-c4ccc(-c5ccc6ccccc6c5)cc4)cc3)c3cccc4sc5ccccc5c34)c3sc4ccccc4c23)cc1 ; c1ccc(-c2ccc(N(c3cccc4oc5ccccc5c34)c3cccc4sc5ccccc5c34)c3sc4ccccc4c23)cc1 ; c1ccc(-c2ccc(N(c3ccc(-c4ccccc4)c4c3sc3ccccc34)c3ccc(-c4ccccc4)c4sc5ccccc5c34)cc2)cc1] 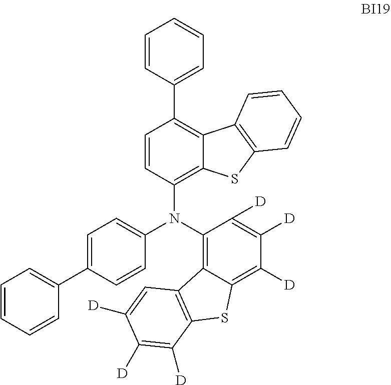 [SMILES: [2H]c1cc2c(sc3c([2H])c([2H])c([2H])c(N(c4ccc(-c5ccccc5)cc4)c4ccc(-c5ccccc5)c5c4sc4ccccc45)c32)c([2H])c1[2H]] 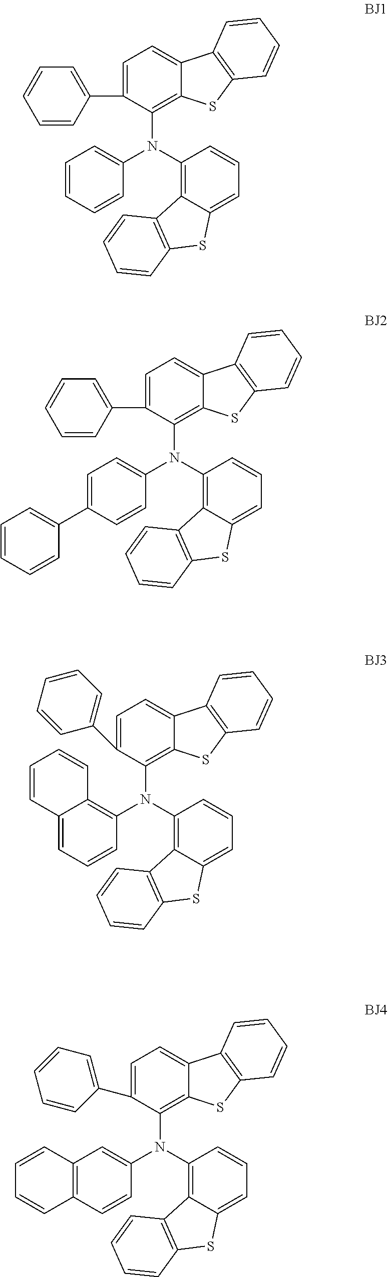 [SMILES: c1ccc(-c2ccc3c(sc4ccccc43)c2N(c2ccccc2)c2cccc3sc4ccccc4c23)cc1 ; c1ccc(-c2ccc(N(c3c(-c4ccccc4)ccc4c3sc3ccccc34)c3cccc4sc5ccccc5c34)cc2)cc1 ; c1ccc(-c2ccc3c(sc4ccccc43)c2N(c2cccc3ccccc23)c2cccc3sc4ccccc4c23)cc1 ; c1ccc(-c2ccc3c(sc4ccccc43)c2N(c2ccc3ccccc3c2)c2cccc3sc4ccccc4c23)cc1] 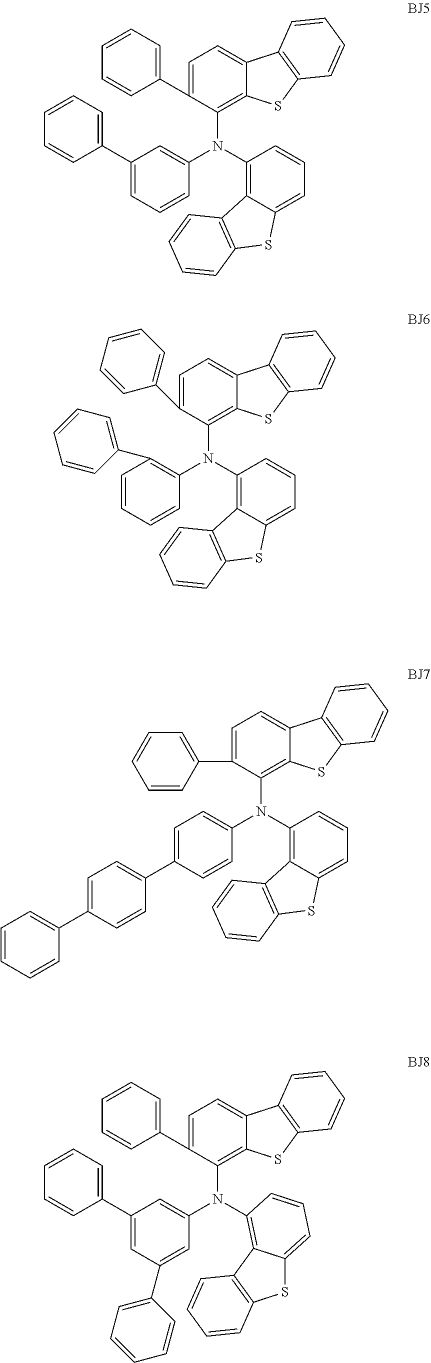 [SMILES: c1ccc(-c2cccc(N(c3c(-c4ccccc4)ccc4c3sc3ccccc34)c3cccc4sc5ccccc5c34)c2)cc1 ; c1ccc(-c2ccccc2N(c2c(-c3ccccc3)ccc3c2sc2ccccc23)c2cccc3sc4ccccc4c23)cc1 ; c1ccc(-c2ccc(-c3ccc(N(c4c(-c5ccccc5)ccc5c4sc4ccccc45)c4cccc5sc6ccccc6c45)cc3)cc2)cc1 ; c1ccc(-c2cc(-c3ccccc3)cc(N(c3c(-c4ccccc4)ccc4c3sc3ccccc34)c3cccc4sc5ccccc5c34)c2)cc1] 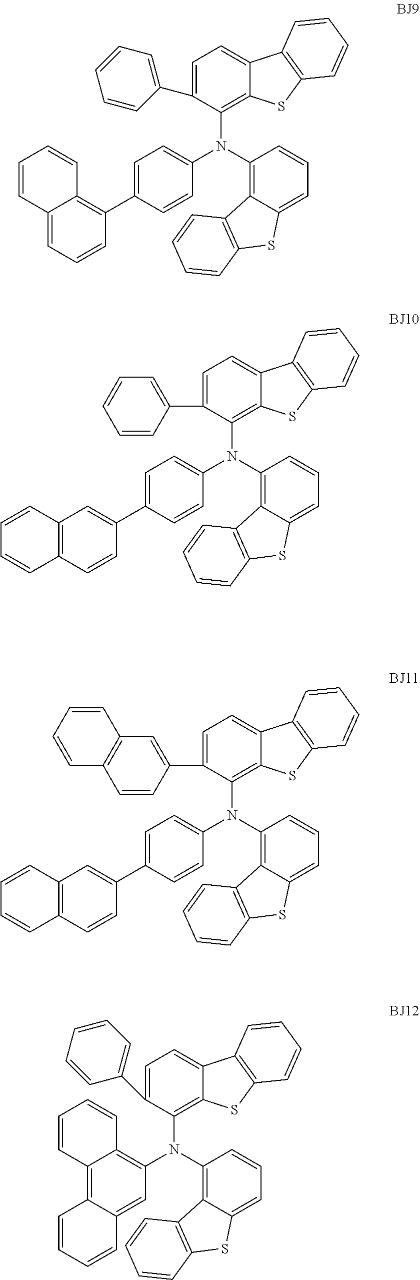 [SMILES: c1ccc(-c2ccc3c(sc4ccccc43)c2N(c2ccc(-c3cccc4ccccc34)cc2)c2cccc3sc4ccccc4c23)cc1 ; c1ccc(-c2ccc3c(sc4ccccc43)c2N(c2ccc(-c3ccc4ccccc4c3)cc2)c2cccc3sc4ccccc4c23)cc1 ; c1ccc2cc(-c3ccc(N(c4c(-c5ccc6ccccc6c5)ccc5c4sc4ccccc45)c4cccc5sc6ccccc6c45)cc3)ccc2c1 ; c1ccc(-c2ccc3c(sc4ccccc43)c2N(c2cc3ccccc3c3ccccc23)c2cccc3sc4ccccc4c23)cc1] 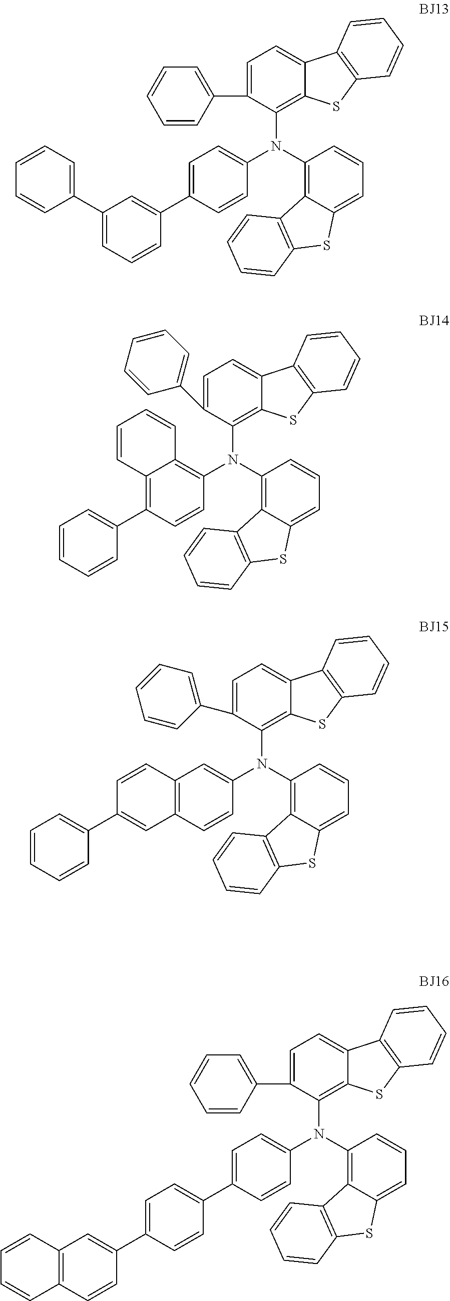 [SMILES: c1ccc(-c2cccc(-c3ccc(N(c4c(-c5ccccc5)ccc5c4sc4ccccc45)c4cccc5sc6ccccc6c45)cc3)c2)cc1 ; c1ccc(-c2ccc3c(sc4ccccc43)c2N(c2ccc(-c3ccccc3)c3ccccc23)c2cccc3sc4ccccc4c23)cc1 ; c1ccc(-c2ccc3cc(N(c4c(-c5ccccc5)ccc5c4sc4ccccc45)c4cccc5sc6ccccc6c45)ccc3c2)cc1 ; c1ccc(-c2ccc3c(sc4ccccc43)c2N(c2ccc(-c3ccc(-c4ccc5ccccc5c4)cc3)cc2)c2cccc3sc4ccccc4c23)cc1] 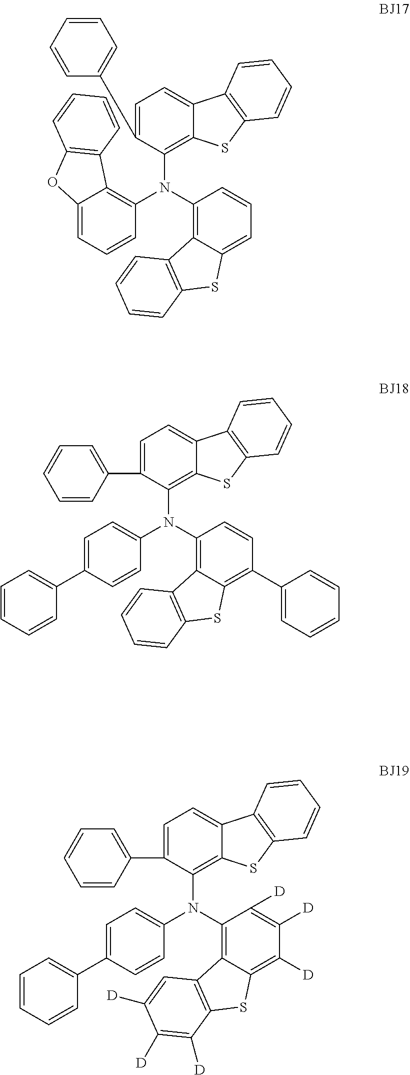 [SMILES: c1ccc(-c2ccc3c(sc4ccccc43)c2N(c2cccc3oc4ccccc4c23)c2cccc3sc4ccccc4c23)cc1 ; c1ccc(-c2ccc(N(c3c(-c4ccccc4)ccc4c3sc3ccccc34)c3ccc(-c4ccccc4)c4sc5ccccc5c34)cc2)cc1 ; [2H]c1cc2c(sc3c([2H])c([2H])c([2H])c(N(c4ccc(-c5ccccc5)cc4)c4c(-c5ccccc5)ccc5c4sc4ccccc45)c32)c([2H])c1[2H]] 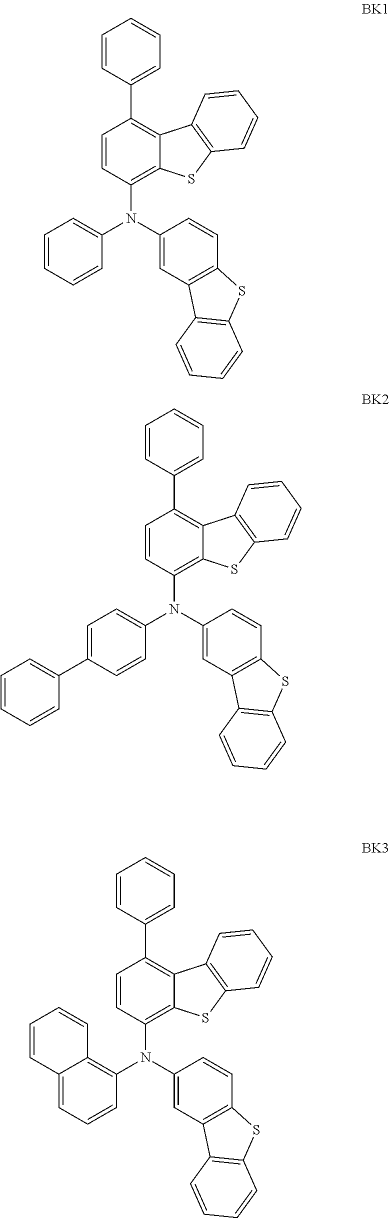 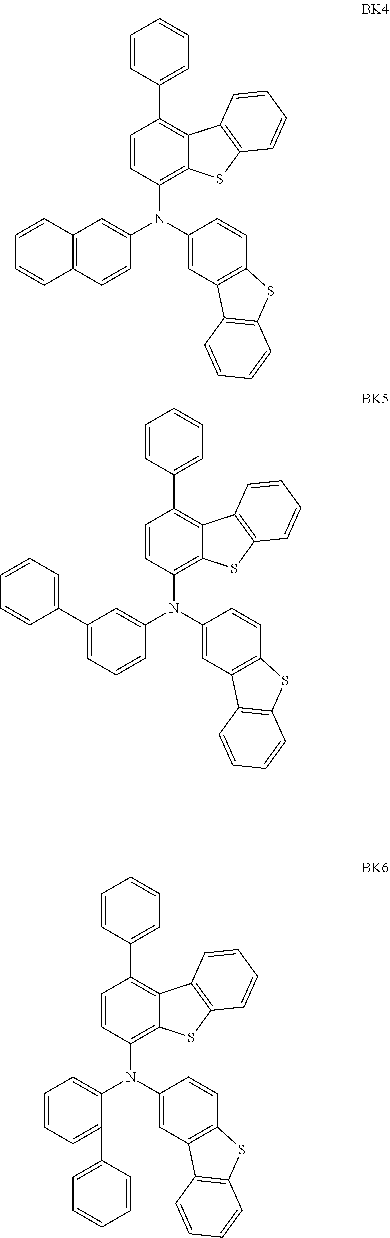 [SMILES: c1ccc(-c2ccc(N(c3ccc4ccccc4c3)c3ccc4sc5ccccc5c4c3)c3sc4ccccc4c23)cc1 ; c1ccc(-c2cccc(N(c3ccc4sc5ccccc5c4c3)c3ccc(-c4ccccc4)c4c3sc3ccccc34)c2)cc1 ; c1ccc(-c2ccccc2N(c2ccc3sc4ccccc4c3c2)c2ccc(-c3ccccc3)c3c2sc2ccccc23)cc1] 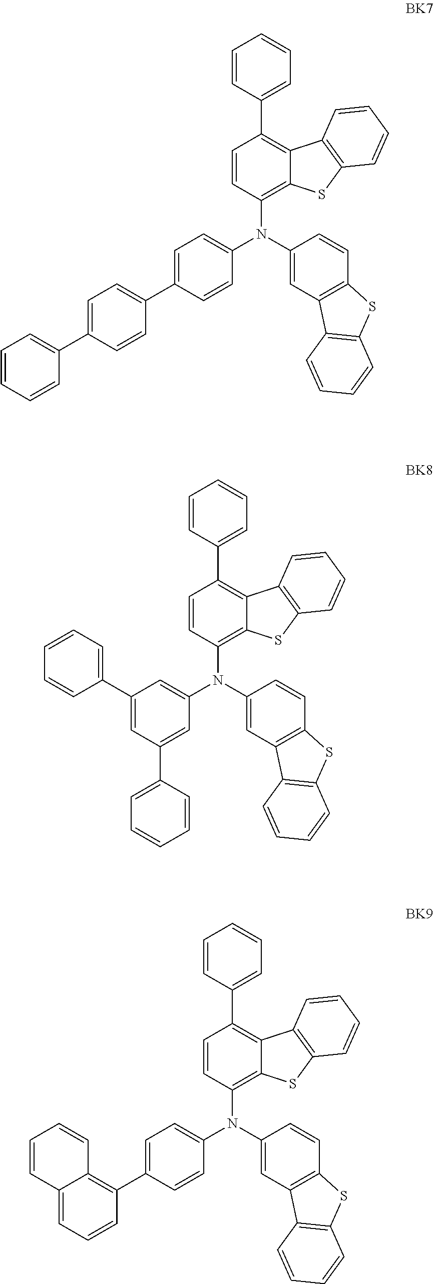 [SMILES: c1ccc(-c2ccc(-c3ccc(N(c4ccc5sc6ccccc6c5c4)c4ccc(-c5ccccc5)c5c4sc4ccccc45)cc3)cc2)cc1 ; c1ccc(-c2cc(-c3ccccc3)cc(N(c3ccc4sc5ccccc5c4c3)c3ccc(-c4ccccc4)c4c3sc3ccccc34)c2)cc1 ; c1ccc(-c2ccc(N(c3ccc(-c4cccc5ccccc45)cc3)c3ccc4sc5ccccc5c4c3)c3sc4ccccc4c23)cc1] 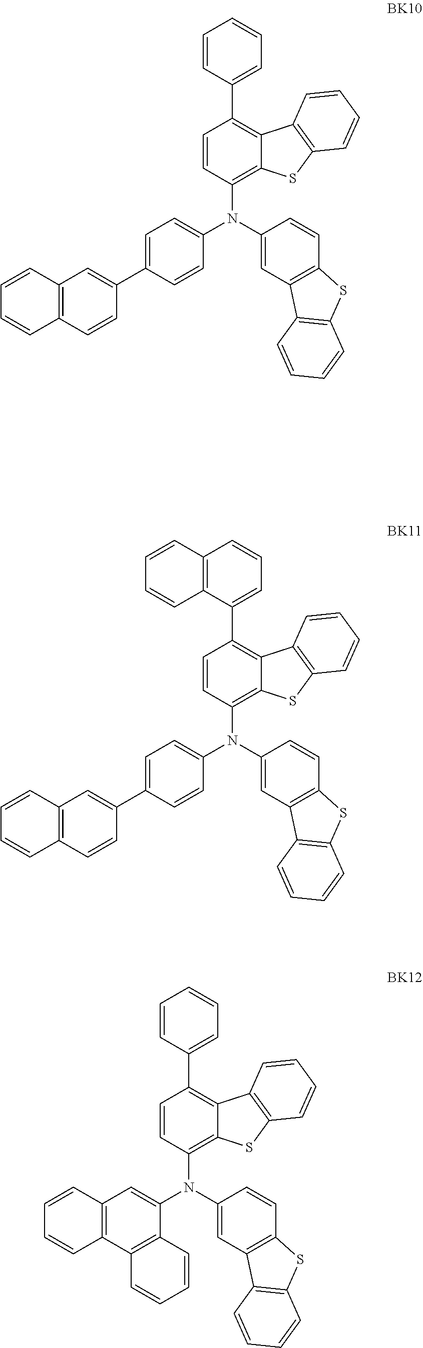 [SMILES: c1ccc(-c2ccc(N(c3ccc(-c4ccc5ccccc5c4)cc3)c3ccc4sc5ccccc5c4c3)c3sc4ccccc4c23)cc1 ; c1ccc2cc(-c3ccc(N(c4ccc5sc6ccccc6c5c4)c4ccc(-c5cccc6ccccc56)c5c4sc4ccccc45)cc3)ccc2c1 ; c1ccc(-c2ccc(N(c3ccc4sc5ccccc5c4c3)c3cc4ccccc4c4ccccc34)c3sc4ccccc4c23)cc1] 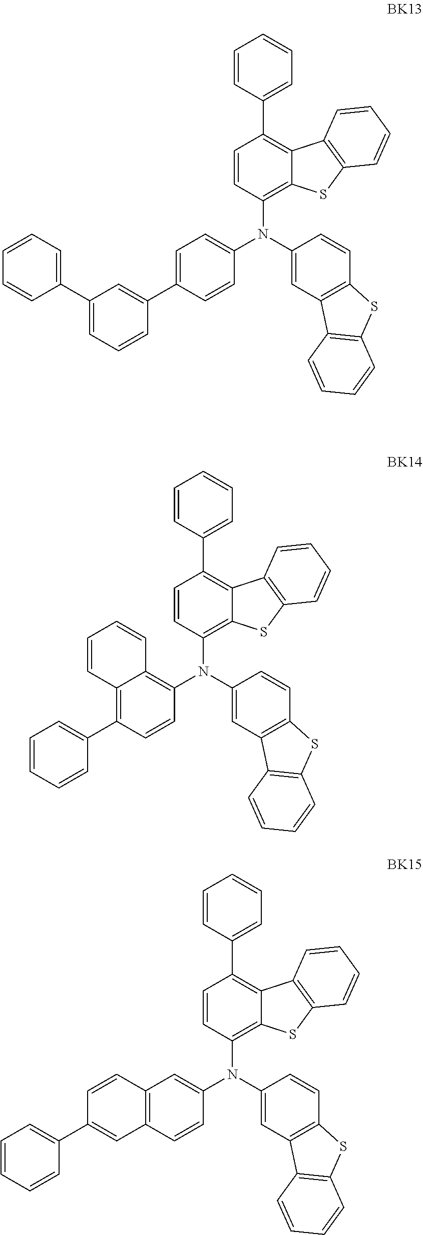 [SMILES: c1ccc(-c2cccc(-c3ccc(N(c4ccc5sc6ccccc6c5c4)c4ccc(-c5ccccc5)c5c4sc4ccccc45)cc3)c2)cc1 ; c1ccc(-c2ccc(N(c3ccc4sc5ccccc5c4c3)c3ccc(-c4ccccc4)c4c3sc3ccccc34)c3ccccc23)cc1 ; c1ccc(-c2ccc3cc(N(c4ccc5sc6ccccc6c5c4)c4ccc(-c5ccccc5)c5c4sc4ccccc45)ccc3c2)cc1] 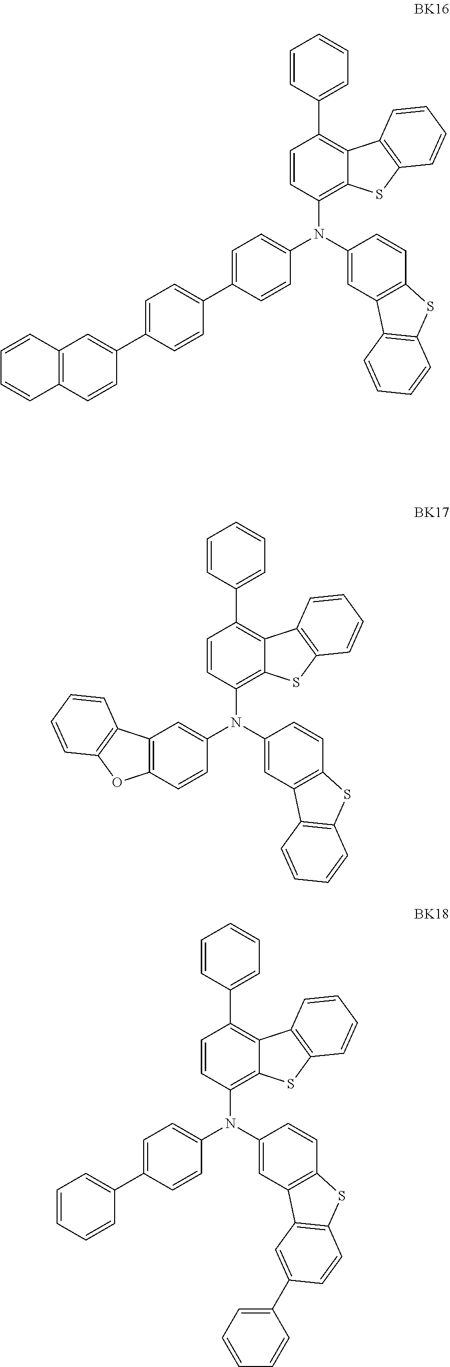 [SMILES: c1ccc(-c2ccc(N(c3ccc(-c4ccc(-c5ccc6ccccc6c5)cc4)cc3)c3ccc4sc5ccccc5c4c3)c3sc4ccccc4c23)cc1 ; c1ccc(-c2ccc(N(c3ccc4oc5ccccc5c4c3)c3ccc4sc5ccccc5c4c3)c3sc4ccccc4c23)cc1 ; c1ccc(-c2ccc(N(c3ccc4sc5ccc(-c6ccccc6)cc5c4c3)c3ccc(-c4ccccc4)c4c3sc3ccccc34)cc2)cc1] 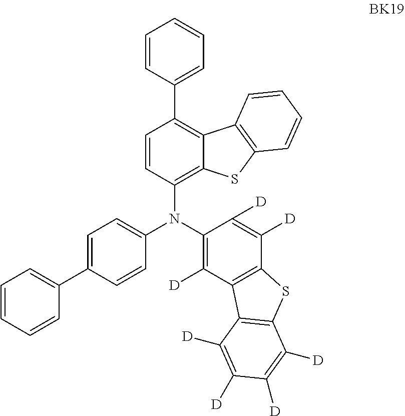 [SMILES: [2H]c1c([2H])c([2H])c2c(sc3c([2H])c([2H])c(N(c4ccc(-c5ccccc5)cc4)c4ccc(-c5ccccc5)c5c4sc4ccccc45)c([2H])c32)c1[2H]] 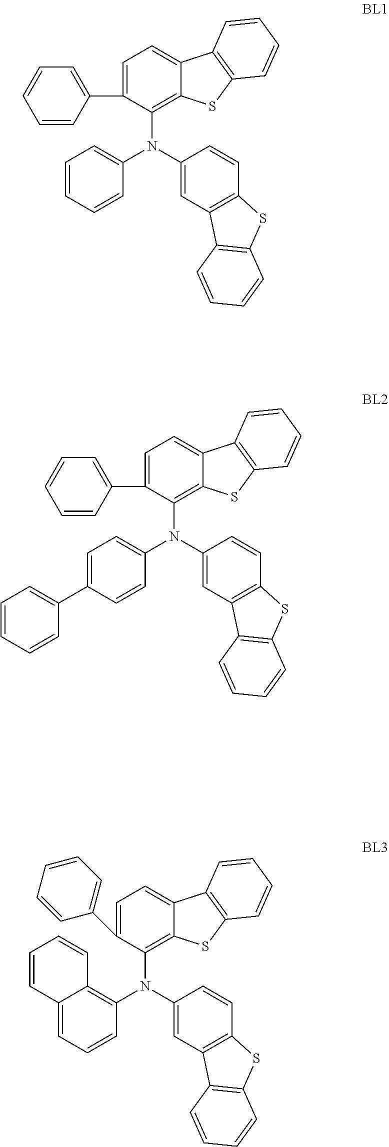 [SMILES: c1ccc(-c2ccc3c(sc4ccccc43)c2N(c2ccccc2)c2ccc3sc4ccccc4c3c2)cc1 ; c1ccc(-c2ccc(N(c3ccc4sc5ccccc5c4c3)c3c(-c4ccccc4)ccc4c3sc3ccccc34)cc2)cc1 ; c1ccc(-c2ccc3c(sc4ccccc43)c2N(c2ccc3sc4ccccc4c3c2)c2cccc3ccccc23)cc1] 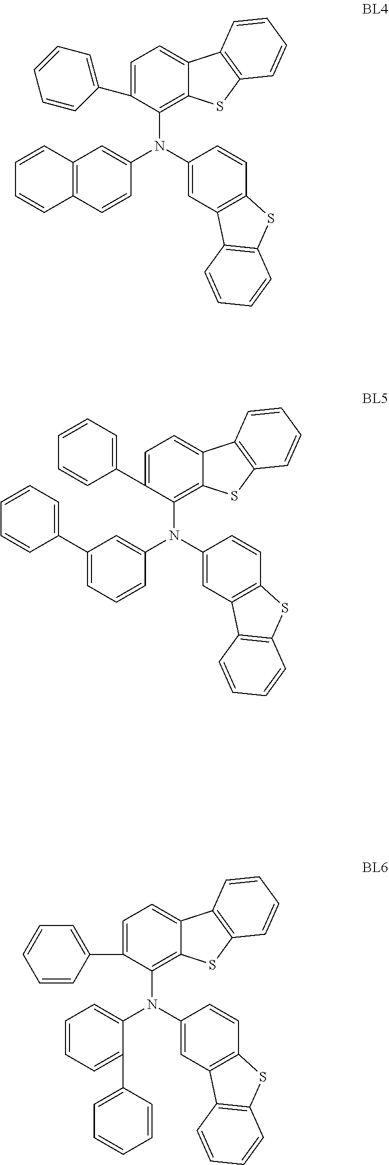 [SMILES: c1ccc(-c2ccc3c(sc4ccccc43)c2N(c2ccc3ccccc3c2)c2ccc3sc4ccccc4c3c2)cc1 ; c1ccc(-c2cccc(N(c3ccc4sc5ccccc5c4c3)c3c(-c4ccccc4)ccc4c3sc3ccccc34)c2)cc1 ; c1ccc(-c2ccccc2N(c2ccc3sc4ccccc4c3c2)c2c(-c3ccccc3)ccc3c2sc2ccccc23)cc1] 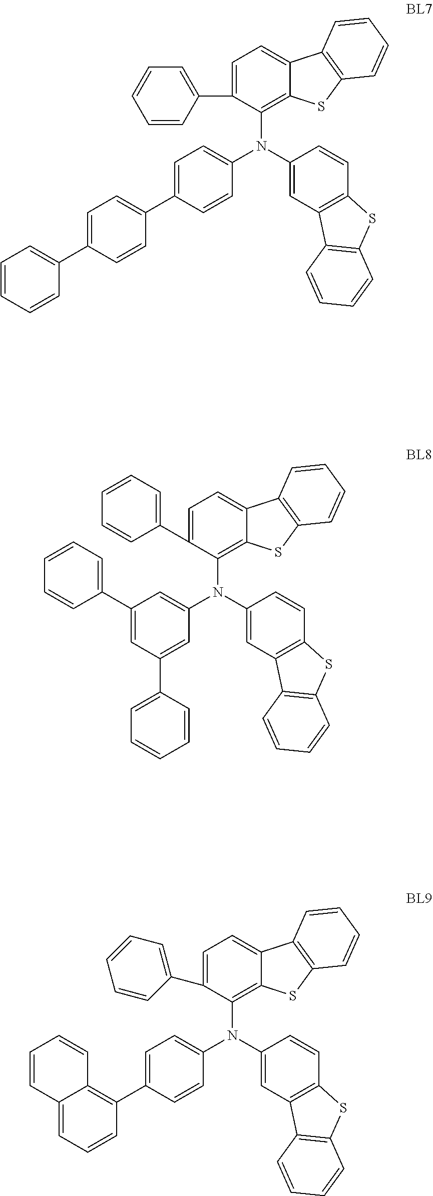 [SMILES: c1ccc(-c2ccc(-c3ccc(N(c4ccc5sc6ccccc6c5c4)c4c(-c5ccccc5)ccc5c4sc4ccccc45)cc3)cc2)cc1 ; c1ccc(-c2cc(-c3ccccc3)cc(N(c3ccc4sc5ccccc5c4c3)c3c(-c4ccccc4)ccc4c3sc3ccccc34)c2)cc1 ; c1ccc(-c2ccc3c(sc4ccccc43)c2N(c2ccc(-c3cccc4ccccc34)cc2)c2ccc3sc4ccccc4c3c2)cc1] 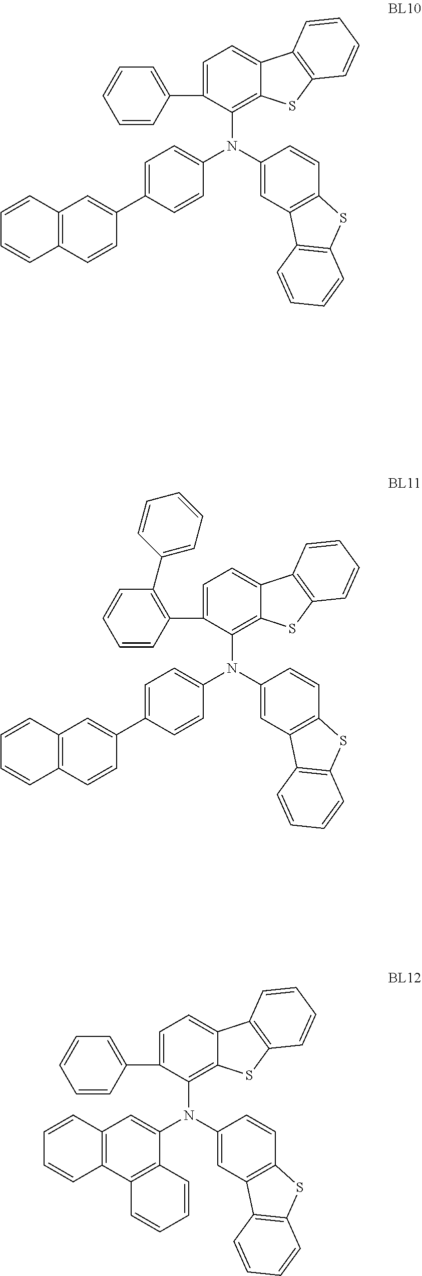 [SMILES: c1ccc(-c2ccc3c(sc4ccccc43)c2N(c2ccc(-c3ccc4ccccc4c3)cc2)c2ccc3sc4ccccc4c3c2)cc1 ; c1ccc(-c2ccccc2-c2ccc3c(sc4ccccc43)c2N(c2ccc(-c3ccc4ccccc4c3)cc2)c2ccc3sc4ccccc4c3c2)cc1 ; c1ccc(-c2ccc3c(sc4ccccc43)c2N(c2ccc3sc4ccccc4c3c2)c2cc3ccccc3c3ccccc23)cc1] 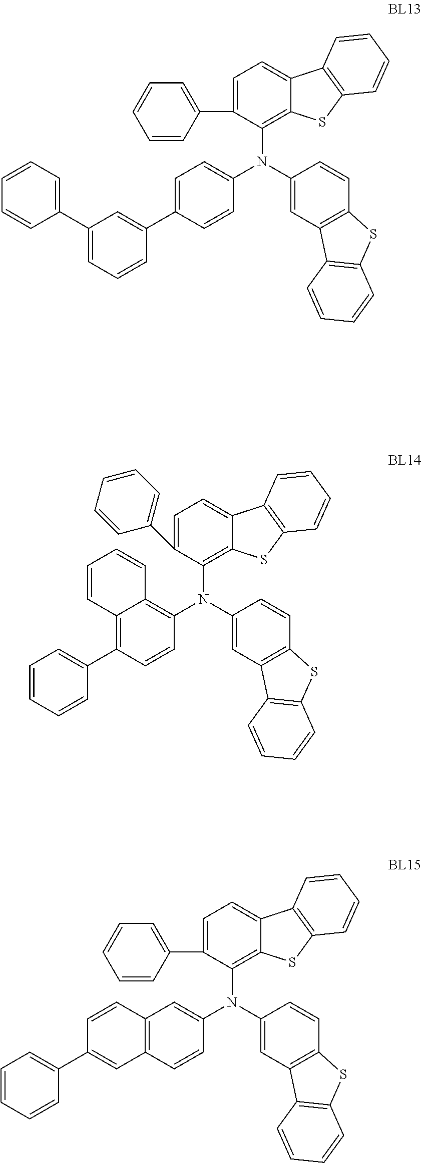 [SMILES: c1ccc(-c2cccc(-c3ccc(N(c4ccc5sc6ccccc6c5c4)c4c(-c5ccccc5)ccc5c4sc4ccccc45)cc3)c2)cc1 ; c1ccc(-c2ccc3c(sc4ccccc43)c2N(c2ccc3sc4ccccc4c3c2)c2ccc(-c3ccccc3)c3ccccc23)cc1 ; c1ccc(-c2ccc3cc(N(c4ccc5sc6ccccc6c5c4)c4c(-c5ccccc5)ccc5c4sc4ccccc45)ccc3c2)cc1] 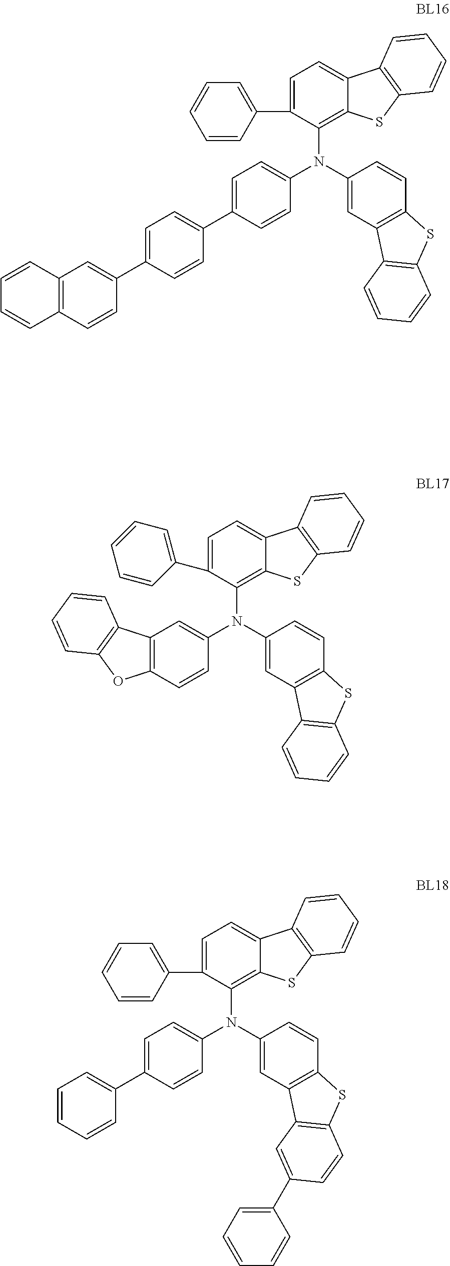 [SMILES: c1ccc(-c2ccc3c(sc4ccccc43)c2N(c2ccc(-c3ccc(-c4ccc5ccccc5c4)cc3)cc2)c2ccc3sc4ccccc4c3c2)cc1 ; c1ccc(-c2ccc3c(sc4ccccc43)c2N(c2ccc3oc4ccccc4c3c2)c2ccc3sc4ccccc4c3c2)cc1 ; c1ccc(-c2ccc(N(c3ccc4sc5ccc(-c6ccccc6)cc5c4c3)c3c(-c4ccccc4)ccc4c3sc3ccccc34)cc2)cc1] [chemical structure]
